(19) United States
(12) Patent Application Publication
(10) Pub. No.: US 2021/0104321 A1
(43) Pub. Date: Apr. 8, 2021

(54) MACHINE LEARNING DISEASE PREDICTION AND TREATMENT PRIORITIZATION

(71) Applicant: AMPEL BioSolutions, LLC, Charlottesville, VA (US)

(72) Inventors: Peter E. LIPSKY, Charlottesville, VA (US); Michelle D. CATALINA, Harvard, MA (US); Amrie C. GRAMMER, Charlottesville, VA (US); Brian KEGERREIS, Durham, NC (US); Adam LABONTE, Palmyra, VA (US); Katherine A. OWEN, Crozet, VA (US); Prathyusha BACHALI, Gaithersburg, MD (US)

(21) Appl. No.: 16/679,109

(22) Filed: Nov. 8, 2019

Related U.S. Application Data
(60) Provisional application No. 62/926,355, filed on Oct. 25, 2019.
(60) Provisional application No. 62/912,560, filed on Oct. 8, 2019.
(60) Provisional application No. 62/881,286, filed on Jul. 31, 2019.
(60) Provisional application No. 62/869,903, filed on Jul. 2, 2019.
(60) Provisional application No. 62/863,772, filed on Jun. 19, 2019.
(60) Provisional application No. 62/863,192, filed on Jun. 18, 2019.
(60) Provisional application No. 62/833,493, filed on Apr. 12, 2019.
(60) Provisional application No. 62/828,895, filed on Apr. 3, 2019.
(60) Provisional application No. 62/768,054, filed on Nov. 15, 2018.

(51) Int. Cl. G16H 50/20; G16B 20/20; G16B 25/10; G16H 10/40; G16H 50/70; G16H 10/20; G16H 50/50; G16H 20/10; G16H 70/40; G16H 70/60; G16H 15/00; G06T 7/00

(52) U.S. Cl. CPC G16H 50/20; G16B 20/20; G16B 25/10; G16H 10/40; G16H 50/70; G16H 10/20; G06T 2207/30084; G16H 20/10; G16H 70/40; G16H 70/60; G16H 15/00; G06T 7/0014; G16H 50/50

(57) ABSTRACT

Described are machine learning methods of identifying one or more records having a specific phenotype to enable proper correlation between genetic records and phenotypes. In an aspect, a method of identifying one or more records having a specific phenotype may comprise: (a) receiving a plurality of first records, each associated with one or more of a plurality of phenotypes; (b) receiving a plurality of second records, each associated with one or more of the phenotypes, wherein the first and second records are non-overlapping; (c) applying a machine learning algorithm to at least one first record and at least one second record to determine a classifier; (d) receiving a plurality of third records, distinct from the first and second records; and (e) applying the classifier to the third records to identify one or more third records associated with the specific phenotype.

### CROSS-REFERENCE

This application claims the benefit of U.S. Provisional Patent Application No. 62/768,054, filed Nov. 15, 2018, U.S. Provisional Patent Application No. 62/828,895, filed Apr. 3, 2019, U.S. Provisional Patent Application No. 62/833,493, filed Apr. 12, 2019, U.S. Provisional Patent Application No. 62/863,192, filed Jun. 18, 2019, U.S. Provisional Patent Application No. 62/863,772, filed Jun. 19, 2019, U.S. Provisional Patent Application No. 62/869,903, filed Jul. 2, 2019, U.S. Provisional Patent Application No. 62/881,286, filed Jul. 31, 2019, U.S. Provisional Patent Application No. 62/912,560, filed Oct. 8, 2019, and U.S. Provisional Patent Application No. 62/926,355, filed Oct. 25, 2019, each of which is entirely incorporated herein by reference.

### BACKGROUND

Machine learning is a computational method capable of harnessing complex data from multiple sources to develop self-trained prediction and analysis tools. When applied to high-scale disease and treatment data, machine learning algorithms may quickly and effectively identify genetic and phenotypic features.

### SUMMARY

### Analysis by Molecular Endotyping

In an aspect, the present disclosure provides a method of identifying one or more records having a specific phenotype, the method comprising: receiving a plurality of first records, wherein each first record is associated with one or more of a plurality of phenotypes; receiving a plurality of second records, wherein each second record is associated with one or more of the plurality of phenotypes, and wherein the plurality of second records and the plurality of first records are non-overlapping; applying a machine learning algorithm to at least one first record and at least one second record to determine a classifier; receiving a plurality of third records, wherein the third records are distinct from the plurality of first records and the plurality of second records; and applying the classifier to the plurality of third records to identify one or more third records associated with the specific phenotype.

In some embodiments, the first records and the second records comprise nucleic acid sequencing data, transcriptome data, genome data, epigenome data, proteome data, metabolome data, virome data, metabolome data, methylome data, lipidomic data, lineage-ome data, nucleosomal occupancy data, a genetic variant, a gene fusion, an insertion or deletion (indel), or any combination thereof. In some embodiments, the first records and the second records are in different formats. In some embodiments, the first records and the second records are from different sources, different studies, or both. In some embodiments, the phenotype comprises a disease state, an organ involvement, a medication response, or any combination thereof. In some embodiments, the classifier comprises an elastic generalized linear model classifier, a k-nearest neighbors classifier, a random forest classifier, or any combination thereof.

In some embodiments, the elastic generalized linear model classifier employs an elastic penalty of about 0.8 to about 1. In some embodiments, the elastic generalized linear model classifier employs an elastic penalty of at least about 0.8, about 0.825, about 0.85, about 0.875, about 0.9, about 0.925, about 0.95, about 0.975, or about 1. In some embodiments, the elastic generalized linear model classifier employs an elastic penalty of at most about 0.8, about 0.825, about 0.85, about 0.875, about 0.9, about 0.925, about 0.95, about 0.975, or about 1. In some embodiments, the elastic generalized linear model classifier employs an elastic penalty of about 0.8 to about 0.825, about 0.8 to about 0.85, about 0.8 to about 0.875, about 0.8 to about 0.9, about 0.8 to about 0.925, about 0.8 to about 0.95, about 0.8 to about 0.975, about 0.8 to about 1, about 0.825 to about 0.85, about 0.825 to about 0.875, about 0.825 to about 0.9, about 0.825 to about 0.925, about 0.825 to about 0.95, about 0.825 to about 0.975, about 0.825 to about 1, about 0.85 to about 0.875, about 0.85 to about 0.9, about 0.85 to about 0.925, about 0.85 to about 0.95, about 0.85 to about 0.975, about 0.85 to about 1, about 0.875 to about 0.9, about 0.875 to about 0.925, about 0.875 to about 0.95, about 0.875 to about 0.975, about 0.875 to about 1, about 0.9 to about 0.925, about 0.9 to about 0.95, about 0.9 to about 0.975, about 0.9 to about 1, about 0.925 to about 0.95, about 0.925 to about 0.975, about 0.925 to about 1, about 0.95 to about 0.975, about 0.95 to about 1, or about 0.975 to about 1. In some embodiments, the elastic generalized linear model classifier employs an elastic penalty of about 0.8, about 0.825, about 0.85, about 0.875, about 0.9, about 0.925, about 0.95, about 0.975, or about 1.

In some embodiments, the k-nearest neighbors classifier employs a K value of the size of the plurality of distinct first data sets, wherein k is about 1 to about 20. In some embodiments, the k-nearest neighbors classifier employs a K value of the size of the plurality of distinct first data sets, wherein k is at least about 1, about 2, about 3, about 4, about 5, about 6, about 8, about 10, about 12, about 14, about 16, or about 20. In some embodiments, the k-nearest neighbors classifier employs a K value of the size of the plurality of distinct first data sets, wherein k is at most about 1, about 2, about 3, about 4, about 5, about 6, about 8, about 10, about 12, about 14, about 16, or about 20. In some embodiments, the k-nearest neighbors classifier employs a K value of the size of the plurality of distinct first data sets, wherein k is about 1 to about 2, about 1 to about 3, about 1 to about 4, about 1 to about 5, about 1 to about 6, about 1 to about 8, about 1 to about 10, about 1 to about 12, about 1 to about 14, about 1 to about 16, about 1 to about 20, about 2 to about 3, about 2 to about 4, about 2 to about 5, about 2 to about 6, about 2 to about 8, about 2 to about 10, about 2 to about 12, about 2 to about 14, about 2 to about 16, about 2 to about 20, about 3 to about 4, about 3 to about 5, about 3 to about 6, about 3 to about 8, about 3 to about 10, about 3 to about 12, about 3 to about 14, about 3 to about 16, about 3 to about 20, about 4 to about 5, about 4 to about 6, about 4 to about 8, about 4 to about 10, about 4 to about 12, about 4 to about 14, about 4 to about 16, about 4 to about 20, about 5 to about 6, about 5 to about 8, about 5 to about 10, about 5 to about 12, about 5 to about 14, about 5 to about 16, about 5 to about 20, about 6 to about 8, about 6 to about 10, about 6 to about 12, about 6 to about 14, about 6 to about 16, about 6 to about 20, about 8 to about 10, about 8 to about 12, about 8 to about 14, about 8 to about 16, about 8 to about 20, about 10 to about 12, about 10 to about 14, about 10 to about 16, about 10 to about 20, about 12 to about 14, about 12 to about 16, about 12 to about 20, about 14 to about 16, about 14 to about 20, or about 16 to about 20. In some embodiments, the k-nearest neighbors classifier employs a K value of the size of the plurality of distinct first data sets, wherein k is about 1, about 2, about 3, about 4, about 5, about 6, about 8, about 10, about 12, about 14, about 16, or about 20.

In some embodiments, the K-value of the random forest classifier is incremented by 1 if the k-value is an even number. In some embodiments, applying a machine learning algorithm to the third data set comprises applying a machine learning algorithm to a plurality of unique third data sets.

In some embodiments, the classifier identifies said one or more third records associated with the specific phenotype with an accuracy of about 70% to about 100%. In some embodiments, the classifier identifies said one or more third records associated with the specific phenotype with an accuracy of at least about 70%, about 75%, about 80%, about 85%, about 90%, about 95%, or about 100%. In some embodiments, the classifier identifies said one or more third records associated with the specific phenotype with an accuracy of at most about 70%, about 75%, about 80%, about 85%, about 90%, about 95%, or about 100%. In some embodiments, the classifier identifies said one or more third records associated with the specific phenotype with an accuracy of about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some embodiments, the classifier identifies said one or more third records associated with the specific phenotype with an accuracy of about 70%, about 75%, about 80%, about 85%, about 90%, about 95%, or about 100%.

In some embodiments, the classifier identifies said one or more third records associated with the specific phenotype with an accuracy of about 70% to about 100%. In some embodiments, the classifier identifies said one or more third records associated with the specific phenotype with an accuracy of at least about 70%, about 75%, about 80%, about 85%, about 90%, about 95%, or about 100%. In some embodiments, the classifier identifies said one or more third records associated with the specific phenotype with an accuracy of at most about 70%, about 75%, about 80%, about 85%, about 90%, about 95%, or about 100%. In some embodiments, the classifier identifies said one or more third records associated with the specific phenotype with an accuracy of about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some embodiments, the classifier identifies said one or more third records associated with the specific phenotype with an accuracy of about 70%, about 75%, about 80%, about 85%, about 90%, about 95%, or about 100%.

In some embodiments, the classifier herein enables a specific phenotype association sensitivity of about 70% to about 100%. In some embodiments, the classifier herein enables a specific phenotype association sensitivity of at least 70%, about 75%, about 80%, about 85%, about 90%, about 95%, or about 100%. In some embodiments, the classifier herein enables a specific phenotype association sensitivity of at most 70%, about 75%, about 80%, about 85%, about 90%, about 95%, or about 100%. In some embodiments, the classifier herein enables a specific phenotype association sensitivity of about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some embodiments, the classifier herein enables a specific phenotype association sensitivity of about 70%, about 75%, about 80%, about 85%, about 90%, about 95%, or about 100%.

In some embodiments, the classifier herein enables a specific phenotype association specificity of about 70% to about 100%. In some embodiments, the classifier herein enables a specific phenotype association specificity of at least 70%, about 75%, about 80%, about 85%, about 90%, about 95%, or about 100%. In some embodiments, the classifier herein enables a specific phenotype association specificity of at most 70%, about 75%, about 80%, about 85%, about 90%, about 95%, or about 100%. In some embodiments, the classifier herein enables a specific phenotype association specificity of about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some embodiments, the classifier herein enables a specific phenotype association specificity of about 70%, about 75%, about 80%, about 85%, about 90%, about 95%, or about 100%.

In some embodiments, the method further comprises filtering the first records, the second records, or both. In some embodiments, the filtering comprises removing outliers, removing background noise, removing data without annotation data, normalizing, scaling, variance correcting, Weighted Gene Co-expression Network Analysis, enrichment analysis, dimensionality reduction, or any combination thereof. In some embodiments, the normalizing is performed by Robust Multi-Array Analysis (RMA), Guanine Cytosine Robust Multi-Array Analysis (GCRMA), Linear Models for Microarray Data, variance stabilizing transformation (VST), normal-exponential quantile correction (NEQC), or any combination thereof. In some embodiments, the variance correction comprises employing a local empirical Bayesian shrinkage, adjusting the p-values for multiple hypothesis testing using the Benjamini-Hochberg correction, and removing all data with a set false discovery rate

In some embodiments, the false discovery rate is about 0.000001 to about 0.2. In some embodiments, the false discovery rate is at least about 0.000001. In some embodiments, the false discovery rate is at most about 0.2. In some embodiments, the false discovery rate is about 0.000001 to about 0.00005, about 0.000001 to about 0.00001, about 0.000001 to about 0.0005, about 0.000001 to about 0.0001, about 0.000001 to about 0.005, about 0.000001 to about 0.001, about 0.000001 to about 0.05, about 0.000001 to about 0.01, about 0.000001 to about 0.2, about 0.00005 to about 0.00001, about 0.00005 to about 0.0005, about 0.00005 to about 0.0001, about 0.00005 to about 0.005, about 0.00005 to about 0.001, about 0.00005 to about 0.05, about 0.00005 to about 0.01, about 0.00005 to about 0.2, about 0.00001 to about 0.0005, about 0.00001 to about 0.0001, about 0.00001 to about 0.005, about 0.00001 to about 0.001, about 0.00001 to about 0.05, about 0.00001 to about 0.01, about 0.00001 to about 0.2, about 0.0005 to about 0.0001, about 0.0005 to about 0.005, about 0.0005 to about 0.001, about 0.0005 to about 0.05, about 0.0005 to about 0.01, about 0.0005 to about 0.2, about 0.0001 to about 0.005, about 0.0001 to about 0.001, about 0.0001 to about 0.05, about 0.0001 to about 0.01, about 0.0001 to about 0.2, about 0.005 to about 0.001, about 0.005 to about 0.05, about 0.005 to about 0.01, about 0.005 to about 0.2, about 0.001 to about 0.05, about 0.001 to about 0.01, about 0.001 to about 0.2, about 0.05 to about 0.01, about 0.05 to about 0.2, or about 0.01 to about 0.2. In some embodiments, the false discovery rate is about 0.000001, about 0.00005, about 0.00001, about 0.0005, about 0.0001, about 0.005, about 0.001, about 0.05, about 0.01, or about 0.2.

In some embodiments, the Weighted Gene Co-expression Network Analysis comprises calculating a topology matrix, clustering the data based on the topology matrix, and correlating module eigenvalues for traits on a linear scale by Pearson correlation, for nonparametric traits by Spearman correlation, and for dichotomous traits by point-biserial correlation or t-test. The Pearson correlation or the Product Moment Correlation Coefficient (PMCC), is a number between −1 and 1 that indicates the extent to which two variables are linearly related. The Spearman correlation is a nonparametric measure of rank correlation; statistical dependence between the rankings of two variables.

In some embodiments, the one or more records having a specific phenotype correspond to one or more subjects, and the method further comprises identifying the one or more subjects as (i) having a diagnosis of a lupus condition, (ii) having a prognosis of a lupus condition, (iii) being suitable or not suitable for enrollment in a clinical trial for a lupus condition, (iv) being suitable or not suitable for being administered a therapeutic regimen configured to treat a lupus condition, (v) having an efficacy or not having an efficacy of a therapeutic regimen configured to treat a lupus condition, based at least in part on the specific phenotype corresponding to the one or more subjects.

In another aspect, the present disclosure provides a non-transitory computer-readable storage media encoded with a computer program including instructions executable by a processor to create an application for identifying one or more records having a specific phenotype, the application comprising: a first receiving module receiving a plurality of first records, wherein each first record is associated with one or more of a plurality of phenotypes; a second receiving module receiving a plurality of second records, wherein each second record is associated with one or more of the plurality of phenotypes, and wherein the plurality of second records and the plurality of first records are non-overlapping; a machine learning module applying a machine learning algorithm to at least one first record and at least one second record to determine a classifier; a third receiving module receiving a plurality of third records, wherein the third records are distinct from the plurality of first records and the plurality of second records; and a classifying module applying the classifier to the plurality of third records to identify one or more third records associated with the specific phenotype.

In some embodiments, the first records and the second records comprise nucleic acid sequencing data, transcriptome data, genome data, epigenome data, proteome data, metabolome data, virome data, metabolome data, methylome data, lipidomic data, lineage-ome data, nucleosomal occupancy data, a genetic variant, a gene fusion, an insertion or deletion (indel), or any combination thereof. In some embodiments, the first records and the second records are in different formats. In some embodiments, the first records and the second records are from different sources, different studies, or both. In some embodiments, the phenotype comprises a disease state, an organ involvement, a medication response, or any combination thereof. In some embodiments, the classifier comprises an elastic generalized linear model classifier, a k-nearest neighbors classifier, a random forest classifier, or any combination thereof. In some embodiments, the elastic generalized linear model classifier employs an elastic penalty of about 0.9. In some embodiments, the k-nearest neighbors classifier employs a K-value of about 5% of the size of the plurality of distinct first data sets. In some embodiments, the K-value of the random forest classifier is incremented by 1 if the k-value is an even number. In some embodiments, applying a machine learning algorithm to the third data set comprises applying a machine learning algorithm to a plurality of unique third data sets. In some embodiments, said classifier identifies said one or more third records associated with the specific phenotype with an accuracy of at least about 70%. In some embodiments, the method further comprises filtering the first records, the second records, or both. In some embodiments, the filtering comprises removing outliers, removing background noise, removing data without annotation data, normalizing, scaling, variance correcting, Weighted Gene Co-expression Network Analysis, enrichment analysis, dimensionality reduction, or any combination thereof. In some embodiments, the normalizing is performed by Robust Multi-Array Analysis (RMA), Guanine Cytosine Robust Multi-Array Analysis (GCRMA), Linear Models for Microarray Data, variance stabilizing transformation (VST), normal-exponential quantile correction (NEQC), or any combination thereof. In some embodiments, the variance correction comprises employing a local empirical Bayesian shrinkage, adjusting the p-values for multiple hypothesis testing using the Benjamini-Hochberg correction, and removing all data with a false discovery rate of less than 0.2. In some embodiments, the Weighted Gene Co-expression Network Analysis comprises calculating a topology matrix, clustering the data based on the topology matrix, and correlating module eigenvalues for traits on a linear scale by Pearson correlation, for nonparametric traits by Spearman correlation, and for dichotomous traits by point-biserial correlation or t-test.

In another aspect, the present disclosure provides a method for identifying a disease state or a susceptibility thereof of a subject, comprising: (a) using an assay to process a biological sample derived from the subject to generate a quantitative measure of each of a plurality of disease-associated genomic loci, wherein the plurality of disease-associated genomic loci comprises at least 5 genes associated with a module of Table 8; (b) processing the dataset to identify the disease state or the susceptibility thereof of the subject at an accuracy of at least about 70%; and (c) electronically outputting a report indicative of the disease state or the susceptibility thereof of the subject.

In some embodiments, the plurality of quantitative measures comprises gene expression measurements. In some embodiments, the disease state comprises an active lupus condition or an inactive lupus condition. In some embodiments, the lupus condition is SLE. In some embodiments, the plurality of disease-associated genomic loci comprises one or more genes selected from the group consisting of: RAB4B, ADAR, MRPL44, CDCA5, MYD88, SNN, BRD3, C7orf43, CDC20, SP1, POFUT1, SAMD4B, ATP6V1B2, TSPAN9, SP140, STK26, IRF4, LCP1, LMO2, SF3B4, HIST2H2AA3, CITED4, ADAMS, TICAM1, and HSD17B7.

In another aspect, the present disclosure provides a method for identifying an immunological state of a subject, comprising: (a) using an assay to process a biological sample derived from the subject to generate a quantitative measure of each of a plurality of genomic loci, wherein the plurality of genomic loci comprises at least 5 genes associated with a module of Table 8; (b) processing the dataset to identify the immunological state of the subject at an accuracy of at least about 70%; and (c) electronically outputting a report indicative of the immunological state of the subject.

In some embodiments, the plurality of quantitative measures comprises gene expression measurements. In some embodiments, the immunological state comprises an active or inactive state of each of one or more of the plurality of genomic loci. In some embodiments, the plurality of genomic loci comprises one or more genes selected from the group consisting of: RAB4B, ADAR, MRPL44, CDCA5, MYD88, SNN, BRD3, C7orf43, CDC20, SP1, POFUT1, SAMD4B, ATP6V1B2, TSPAN9, SP140, STK26, IRF4, LCP1, LMO2, SF3B4, HIST2H2AA3, CITED4, ADAMS, TICAM1, and HSD17B7.

In another aspect, the present disclosure provides a method for identifying a disease state or a susceptibility thereof of a subject, comprising: (a) using an assay to process a biological sample derived from the subject to generate a quantitative measure of each of a plurality of disease-associated genomic loci, wherein the plurality of disease-associated genomic loci comprises one or more genes associated with a gene cluster of Table 1 to Table 72C; (b) processing the dataset to identify the disease state or the susceptibility thereof of the subject at an accuracy of at least about 70%; and (c) electronically outputting a report indicative of the disease state or the susceptibility thereof of the subject.

In some embodiments, the plurality of quantitative measures comprises gene expression measurements. In some embodiments, the disease state comprises an active lupus condition or an inactive lupus condition. In some embodiments, the lupus condition is systemic lupus erythematosus (SLE), discoid lupus erythematosus (DLE), or lupus nephritis (LN). In some embodiments, the plurality of disease-associated genomic loci comprises 5, 10, 15, 20, 25, 30, 35, 40, 45, 50, or more than 50 genes associated with the gene cluster.

In another aspect, the present disclosure provides a method for identifying an immunological state of a subject, comprising: (a) using an assay to process a biological sample derived from the subject to generate a quantitative measure of each of a plurality of disease-associated genomic loci, wherein the plurality of disease-associated genomic loci comprises one or more genes associated with a gene cluster of Table 1 to Table 72C; (b) processing the dataset to identify the immunological state of the subject at an accuracy of at least about 70%; and (c) electronically outputting a report indicative of the immunological state of the subject.

In some embodiments, the plurality of quantitative measures comprises gene expression measurements. In some embodiments, the immunological state comprises an active lupus condition or an inactive lupus condition. In some embodiments, the lupus condition is systemic lupus erythematosus (SLE), discoid lupus erythematosus (DLE), or lupus nephritis (LN). In some embodiments, the plurality of disease-associated genomic loci comprises 5, 10, 15, 20, 25, 30, 35, 40, 45, 50, or more than 50 genes associated with the gene cluster.

In another aspect, the present disclosure provides a method for identifying an immunological state of a subject, comprising: (a) using an assay to process a biological sample derived from the subject to generate a quantitative measure of each of a plurality of disease-associated genomic loci, wherein the plurality of disease-associated genomic loci comprises one or more genes associated with a pathway of Table 1 to Table 72C; (b) processing the dataset to identify the immunological state of the subject at an accuracy of at least about 70%; and (c) electronically outputting a report indicative of the immunological state of the subject.

In some embodiments, the plurality of quantitative measures comprises gene expression measurements. In some embodiments, the immunological state comprises an active lupus condition or an inactive lupus condition. In some embodiments, the lupus condition is systemic lupus erythematosus (SLE), discoid lupus erythematosus (DLE), or lupus nephritis (LN). In some embodiments, the plurality of disease-associated genomic loci comprises 5, 10, 15, 20, 25, 30, 35, 40, 45, 50, or more than 50 genes associated with the pathway.

### Interferon Profiling of Lupus Conditions

In another aspect, the present disclosure provides a method for identifying a lupus condition of a subject, comprising: (a) assaying a biological sample of the subject to generate a dataset comprising gene expression data; (b) processing the dataset at each of a plurality of genes to determine quantitative measures of each of the plurality of genes, wherein the plurality of genes comprises genes induced by a plurality of interferons, thereby producing an interferon signature of the biological sample of the subject; (c) comparing the interferon signature with one or more reference interferon signatures, wherein the comparing comprises, for at least one of the plurality of genes, comparing the quantitative measure of the gene of the interferon signature with corresponding quantitative measures of the gene of the one or more reference interferon signatures; and (d) based at least in part on the comparison in (c), identifying the lupus condition of the subject.

In some embodiments, the lupus condition is selected from the group consisting of: systemic lupus erythematosus (SLE), discoid lupus erythematosus (DLE), and lupus nephritis (LN). In some embodiments, the biological sample is selected from the group consisting of: a whole blood (WB) sample, a peripheral blood mononuclear cell (PBMC) sample, a tissue sample, and a purified cell sample. In some embodiments, the tissue sample is selected from the group consisting of: skin tissue, synovium tissue, and kidney tissue. In some embodiments, the kidney tissue is selected from the group consisting of: glomerulus (Glom) and tubulointerstitium (TI). In some embodiments, the purified sample is selected from the group consisting of: purified CD4⁺ T cells, purified CD19⁺ B cells, and purified CD14⁺ monocytes.

In some embodiments, the method further comprises purifying a whole blood sample of the subject to obtain the purified cell sample. In some embodiments, assaying the biological sample comprises (i) using a microarray to generate the dataset comprising the gene expression data (ii) sequencing the biological sample to generate the dataset comprising the gene expression data, or (iii) performing quantitative polymerase chain reaction (qPCR) of the biological sample to generate the dataset comprising the gene expression data.

In some embodiments, the plurality of interferons comprises Type I interferons and/or Type II interferons. In some embodiments, the Type I interferons and/or Type II interferons are selected from the group consisting of IFNA2, IFNB1, IFNW1, and IFNG. In some embodiments, the plurality of genes comprises one or more genes induced by in vitro stimulation of PBMC by the plurality of interferons. In some embodiments, the one or more genes induced by in vitro stimulation of PBMC are selected from the genes listed in Table 20. In some embodiments, the one or more genes induced by in vitro stimulation of PBMC are selected from the genes listed in Table 21. In some embodiments, the one or more genes induced by in vitro stimulation of PBMC are selected from the genes listed in Table 22. In some embodiments, the one or more genes induced by in vitro stimulation of PBMC are selected from the genes listed in Table 23. In some embodiments, the plurality of genes comprises one or more genes induced by in vitro stimulation of PBMC by IL12 treatment or TNF treatment. In some embodiments, the one or more genes induced by in vitro stimulation of PBMC are selected from the genes listed in Table 24. In some embodiments, the one or more genes induced by in vitro stimulation of PBMC are selected from the genes listed in Table 25. In some embodiments, the plurality of genes comprises one or more genes induced in vivo in IFNA2-treated HepC patients and/or IFNB1-treated MS patients. In some embodiments, the one or more genes induced in vivo in IFNA2-treated HepC patients and/or IFNB1-treated MS patients are selected from the genes listed in Table 32.

In some embodiments, the quantitative measures of each of the plurality of genes comprise enrichment scores of each of the plurality of genes. In some embodiments, the enrichment scores of each of the plurality of genes comprise gene set variation analysis (GSVA) enrichment scores of each of the plurality of genes.

In some embodiments, (c) further comprises, for the at least one of the plurality of genes, determining a difference between the quantitative measure of the gene of the interferon signature with the corresponding quantitative measures of the gene of the one or more reference interferon signatures. In some embodiments, (d) further comprises identifying the lupus condition of the subject when the difference satisfies a pre-determined criterion. In some embodiments, (c) further comprises, for the at least one of the plurality of genes, determining a Z-score of the quantitative measure of the gene of the interferon signature relative to the corresponding quantitative measures of the gene of the one or more reference interferon signatures. In some embodiments, (d) further comprises identifying the lupus condition of the subject when the Z-score satisfies a pre-determined criterion. In some embodiments, (d) further comprises identifying the lupus condition of the subject when the Z-score is at least 2, and identifying an absence of the lupus condition of the subject when the Z-score is less than 2.

In some embodiments, the method further comprises identifying the lupus condition of the subject at a sensitivity of at least about 70%. In some embodiments, the method further comprises identifying the lupus condition of the subject at a sensitivity of at least about 80%. In some embodiments, the method further comprises identifying the lupus condition of the subject at a sensitivity of at least about 90%.

In some embodiments, the method further comprises identifying the lupus condition of the subject at a specificity of at least about 70%. In some embodiments, the method further comprises identifying the lupus condition of the subject at a specificity of at least about 80%. In some embodiments, the method further comprises identifying the lupus condition of the subject at a specificity of at least about 90%.

In some embodiments, the method further comprises identifying the lupus condition of the subject at a positive predictive value (PPV) of at least about 70%. In some embodiments, the method further comprises identifying the lupus condition of the subject at a positive predictive value (PPV) of at least about 80%. In some embodiments, the method further comprises identifying the lupus condition of the subject at a positive predictive value (PPV) of at least about 90%.

In some embodiments, the method further comprises identifying the lupus condition of the subject at a negative predictive value (NPV) of at least about 70%. In some embodiments, the method further comprises identifying the lupus condition of the subject at a negative predictive value (NPV) of at least about 80%. In some embodiments, the method further comprises identifying the lupus condition of the subject at a negative predictive value (NPV) of at least about 90%.

In some embodiments, the method further comprises identifying the lupus condition of the subject with an Area Under Curve (AUC) of at least about 0.70. In some embodiments, the method further comprises identifying the lupus condition of the subject with an Area Under Curve (AUC) of at least about 0.80. In some embodiments, the method further comprises identifying the lupus condition of the subject with an Area Under Curve (AUC) of at least about 0.90.

In some embodiments, the method further comprises determining or predicting an active or inactive state of the identified lupus condition of the subject. In some embodiments, (d) further comprises identifying the lupus condition of the subject based at least in part on a SLEDAI (sysmetic lupus erythematosus activity index) score of the subject. In some embodiments, the subject is asymptomatic for one or more lupus conditions selected from the group consisting of: systemic lupus erythematosus (SLE), discoid lupus erythematosus (DLE), and lupus nephritis (LN).

In some embodiments, the method further comprises applying a trained algorithm to the interferon signature to identify the lupus condition of the subject. In some embodiments, the trained algorithm is trained using a first set of independent training samples associated with a presence of the lupus condition and a second set of independent training samples associated with an absence of the lupus condition. In some embodiments, the method further comprises using the trained algorithm to process a set of clinical health data of the subject to identify the lupus condition. In some embodiments, the trained algorithm comprises a supervised machine learning algorithm. In some embodiments, the supervised machine learning algorithm comprises a deep learning algorithm, a support vector machine (SVM), a neural network, or a Random Forest.

In some embodiments, (a) comprises (i) subjecting the biological sample to conditions that are sufficient to isolate, enrich, or extract a plurality of nucleic acid molecules; and (ii) analyzing the plurality of nucleic acid molecules to generate the dataset comprising the gene expression data. In some embodiments, the method further comprises using probes configured to selectively enrich the plurality of nucleic acid molecules corresponding to a panel of one or more genomic loci. In some embodiments, the probes are nucleic acid primers. In some embodiments, the probes have sequence complementarity with nucleic acid sequences of the panel of the one or more genomic loci. In some embodiments, the panel of the one or more genomic loci comprises genomic loci corresponding to the plurality of genes. In some embodiments, the panel of the one or more genomic loci comprises at least 5 distinct genomic loci. In some embodiments, the panel of the one or more genomic loci comprises at least 10 distinct genomic loci.

In some embodiments, the method further comprises (e) assaying a second biological sample of the subject to generate a second dataset comprising gene expression data; (f) processing the second dataset at each of the plurality of genes to determine second quantitative measures of each of the plurality of genes, thereby producing a second interferon signature of the second biological sample of the subject; (g) comparing the second interferon signature with one or more reference interferon signatures, wherein the comparing comprises, for at least one of the plurality of genes, comparing the quantitative measure of the gene of the second interferon signature with corresponding quantitative measures of the gene of the one or more reference interferon signatures; and (h) based at least in part on the comparison in (g), identifying the lupus condition of the subject.

In some embodiments, the biological sample and the second biological sample comprise two different sample types selected from the group consisting of: a whole blood (WB) sample, a PBMC sample, a skin tissue sample, a synovium tissue sample, a kidney tissue sample comprising glomerulus (Glom), a kidney tissue sample comprising tubulointerstitium (TI), a purified CD4⁺ T cell sample, a purified CD19⁺ B cell sample, and a purified CD14⁺ monocyte sample.

In some embodiments, the method further comprises determining a likelihood of the identification of the lupus condition of the subject. In some embodiments, the method further comprises providing a therapeutic intervention for the lupus condition of the subject.

In some embodiments, the method further comprises monitoring the lupus condition of the subject, wherein the monitoring comprises assessing the lupus condition of the subject at a plurality of time points, wherein the assessing is based at least on the lupus condition identified in (d) at each of the plurality of time points. In some embodiments, a difference in the assessment of the lupus condition of the subject among the plurality of time points is indicative of one or more clinical indications selected from the group consisting of: (i) a diagnosis of the lupus condition of the subject, (ii) a prognosis of the lupus condition of the subject, and (iii) an efficacy or non-efficacy of a course of treatment for treating the lupus condition of the subject.

In some embodiments, the one or more reference interferon signatures are generated by: assaying a biological sample of one or more patients with dermatomyositis to generate a reference dataset comprising gene expression data; and processing the reference dataset at each of the plurality of genes to determine quantitative measures of each of the plurality of genes.

In another aspect, the present disclosure provides a computer system for identifying a lupus condition of a subject, comprising: a database that is configured to store a dataset comprising gene expression data, wherein the gene expression data is obtained by assaying a biological sample of the subject; and one or more computer processors operatively coupled to the database, wherein the one or more computer processors are individually or collectively programmed to: (i) process the dataset at each of a plurality of genes to determine quantitative measures of each of the plurality of genes, wherein the plurality of genes comprises genes induced by a plurality of interferons, thereby producing an interferon signature of the biological sample of the subject; (ii) compare the interferon signature with one or more reference interferon signatures, wherein the comparing comprises, for at least one of the plurality of genes, comparing the quantitative measure of the gene of the interferon signature with corresponding quantitative measures of the gene of the one or more reference interferon signatures; and (iii) based at least in part on the comparison in (ii), identify the lupus condition of the subject.

In some embodiments, the computer system further comprises an electronic display operatively coupled to the one or more computer processors, wherein the electronic display comprises a graphical user interface that is configured to display the report.

In another aspect, the present disclosure provides a non-transitory computer readable medium comprising machine-executable code that, upon execution by one or more computer processors, implements a method for identifying a lupus condition of a subject, the method comprising: (a) assaying a biological sample of the subject to generate a dataset comprising gene expression data; (b) processing the dataset at each of a plurality of genes to determine quantitative measures of each of the plurality of genes, wherein the plurality of genes comprises genes induced by a plurality of interferons, thereby producing an interferon signature of the biological sample of the subject; (c) comparing the interferon signature with one or more reference interferon signatures, wherein the comparing comprises, for at least one of the plurality of genes, comparing the quantitative measure of the gene of the interferon signature with corresponding quantitative measures of the gene of the one or more reference interferon signatures; and (d) based at least in part on the comparison in (c), identifying the lupus condition of the subject.

In another aspect, the present disclosure provides a method for identifying a sepsis condition of a subject, comprising: (a) assaying a biological sample of the subject to generate a dataset comprising gene expression data; (b) processing the dataset at each of a plurality of genes to determine quantitative measures of each of the plurality of genes, wherein the plurality of genes comprises genes induced by TNF, thereby producing a TNF signature of the biological sample of the subject; (c) comparing the TNF signature with one or more reference TNF signatures, wherein the comparing comprises, for at least one of the plurality of genes, comparing the quantitative measure of the gene of the TNF signature with corresponding quantitative measures of the gene of the one or more reference TNF signatures; and (d) based at least in part on the comparison in (c), identifying the sepsis condition of the subject.

### Low-Density Granulocyte (LDG) Profiling of Lupus Conditions

In another aspect, the present disclosure provides a method for identifying a lupus condition of a subject, comprising: (a) assaying a biological sample of the subject to generate a dataset comprising gene expression data; (b) processing the dataset at each of a plurality of genes to determine quantitative measures of each of the plurality of genes, wherein the plurality of genes comprises low-density granulocyte (LDG)-associated genes, thereby producing an LDG signature of the biological sample of the subject; (c) comparing the LDG signature with one or more reference LDG signatures, wherein the comparing comprises, for at least one of the plurality of genes, comparing the quantitative measure of the gene of the LDG signature with corresponding quantitative measures of the gene of the one or more reference LDG signatures; (d) based at least in part on the comparison in (c), identifying the lupus condition of the subject.

In some embodiments, the lupus condition is selected from the group consisting of: systemic lupus erythematosus (SLE), discoid lupus erythematosus (DLE), and lupus nephritis (LN). In some embodiments, the biological sample is selected from the group consisting of: a whole blood (WB) sample, a PBMC sample, a tissue sample, and a cell sample. In some embodiments, the tissue sample is selected from the group consisting of: skin tissue, synovium tissue, kidney tissue, and bone marrow tissue. In some embodiments, the kidney tissue is selected from the group consisting of: glomerulus (Glom) and tubulointerstitium (TI). In some embodiments, the cell sample is selected from the group consisting of: myelocytes (MY), promyelocytes (PM), polymorphonuclear neutrophils (PMN), and peripheral blood mononuclear cells (PBMC).

In some embodiments, the method further comprises enriching or purifying a whole blood sample of the subject to obtain the cell sample. In some embodiments, assaying the biological sample comprises (i) using a microarray to generate the dataset comprising the gene expression data, (ii) sequencing the biological sample to generate the dataset comprising the gene expression data, or (iii) performing quantitative polymerase chain reaction (qPCR) of the biological sample to generate the dataset comprising the gene expression data.

In some embodiments, the plurality of genes comprises LDG-associated genes selected from the genes listed in Table 33. In some embodiments, the plurality of genes comprises LDG-associated genes selected from the genes listed in Table 34. In some embodiments, the plurality of genes comprises LDG-associated genes selected from the genes listed in Table 42A or Table 42B. In some embodiments, the plurality of genes comprises LDG-associated genes selected from the genes listed in Table 43A-43C. In some embodiments, the plurality of genes comprises LDG-associated genes selected from the genes listed in Table 44A. In some embodiments, the plurality of genes comprises LDG-associated genes selected from the genes listed in Table 45A or Table 45B.

In some embodiments, the quantitative measures of each of the plurality of genes comprise enrichment scores of each of the plurality of genes. In some embodiments, the enrichment scores of each of the plurality of genes comprise gene set variation analysis (GSVA) enrichment scores of each of the plurality of genes. In some embodiments, (c) further comprises, for the at least one of the plurality of genes, determining a difference between the quantitative measure of the gene of the LDG signature with the corresponding quantitative measures of the gene of the one or more reference LDG signatures. In some embodiments, (d) further comprises identifying the lupus condition of the subject when the difference satisfies a pre-determined criterion.

In some embodiments, (c) further comprises, for the at least one of the plurality of genes, determining a Z-score of the quantitative measure of the gene of the LDG signature relative to the corresponding quantitative measures of the gene of the one or more reference LDG signatures. In some embodiments, (d) further comprises identifying the lupus condition of the subject when the Z-score satisfies a pre-determined criterion. In some embodiments, (d) further comprises identifying the lupus condition of the subject when the Z-score is at least 2, and identifying an absence of the lupus condition of the subject when the Z-score is less than 2.

In some embodiments, the method further comprises identifying the lupus condition of the subject at a sensitivity of at least about 70%. In some embodiments, the method further comprises identifying the lupus condition of the subject at a sensitivity of at least about 80%. In some embodiments, the method further comprises identifying the lupus condition of the subject at a sensitivity of at least about 90%.

In some embodiments, the method further comprises identifying the lupus condition of the subject at a specificity of at least about 70%. In some embodiments, the method further comprises identifying the lupus condition of the subject at a specificity of at least about 80%. In some embodiments, the method further comprises identifying the lupus condition of the subject at a specificity of at least about 90%.

In some embodiments, the method further comprises identifying the lupus condition of the subject at a positive predictive value (PPV) of at least about 70%. In some embodiments, the method further comprises identifying the lupus condition of the subject at a positive predictive value (PPV) of at least about 80%. In some embodiments, the method further comprises identifying the lupus condition of the subject at a positive predictive value (PPV) of at least about 90%.

In some embodiments, the method further comprises identifying the lupus condition of the subject at a negative predictive value (NPV) of at least about 70%. In some embodiments, the method further comprises identifying the lupus condition of the subject at a negative predictive value (NPV) of at least about 80%. In some embodiments, the method further comprises identifying the lupus condition of the subject at a negative predictive value (NPV) of at least about 90%.

In some embodiments, the method further comprises identifying the lupus condition of the subject with an Area Under Curve (AUC) of at least about 0.70. In some embodiments, the method further comprises identifying the lupus condition of the subject with an Area Under Curve (AUC) of at least about 0.80. In some embodiments, the method further comprises identifying the lupus condition of the subject with an Area Under Curve (AUC) of at least about 0.90.

In some embodiments, (d) further comprises identifying the lupus condition of the subject based at least in part on a SLEDAI score of the subject. In some embodiments, the subject is asymptomatic for one or more lupus conditions selected from the group consisting of: systemic lupus erythematosus (SLE), discoid lupus erythematosus (DLE), and lupus nephritis (LN).

In some embodiments, the method further comprises applying a trained algorithm to the LDG signature to identify the lupus condition of the subject. In some embodiments, the trained algorithm is trained using a first set of independent training samples associated with a presence of the lupus condition and a second set of independent training samples associated with an absence of the lupus condition. In some embodiments, the method further comprises using the trained algorithm to process a set of clinical health data of the subject to identify the lupus condition. In some embodiments, the trained algorithm comprises a supervised machine learning algorithm. In some embodiments, the supervised machine learning algorithm comprises a deep learning algorithm, a support vector machine (SVM), a neural network, or a Random Forest.

In some embodiments, (a) comprises (i) subjecting the biological sample to conditions that are sufficient to isolate, enrich, or extract a plurality of nucleic acid molecules; and (ii) analyzing the plurality of nucleic acid molecules to generate the dataset comprising the gene expression data.

In some embodiments, the method further comprises using probes configured to selectively enrich the plurality of nucleic acid molecules corresponding to a panel of one or more genomic loci. In some embodiments, the probes are nucleic acid primers. In some embodiments, the probes have sequence complementarity with nucleic acid sequences of the panel of the one or more genomic loci. In some embodiments, the panel of the one or more genomic loci comprises genomic loci corresponding to the plurality of genes. In some embodiments, the panel of said one or more genomic loci comprises at least 5 distinct genomic loci. In some embodiments, the panel of said one or more genomic loci comprises at least 10 distinct genomic loci.

In some embodiments, the method further comprises (e) assaying a second biological sample of the subject to generate a second dataset comprising gene expression data; (f) processing the second dataset at each of the plurality of genes to determine second quantitative measures of each of the plurality of genes, thereby producing a second LDG signature of the second biological sample of the subject; (g) comparing the second LDG signature with one or more reference LDG signatures, wherein the comparing comprises, for at least one of the plurality of genes, comparing the quantitative measure of the gene of the second LDG signature with corresponding quantitative measures of the gene of the one or more reference LDG signatures; and (h) based at least in part on the comparison in (g), identifying the lupus condition of the subject.

In some embodiments, the biological sample and the second biological sample comprise two different sample types selected from the group consisting of: a whole blood (WB) sample, a PBMC sample, a skin tissue sample, a synovium tissue sample, a kidney tissue sample comprising glomerulus (Glom), a kidney tissue sample comprising tubulointerstitium (TI), a bone marrow tissue, a myelocyte (MY) cell sample, a promyelocyte (PM) cell sample, and a polymorphonuclear neutrophils (PMN) sample.

In some embodiments, the method further comprises determining a likelihood of the identification of the lupus condition of the subject. In some embodiments, the method further comprises providing a therapeutic intervention for the lupus condition of the subject.

In some embodiments, the method further comprises monitoring the lupus condition of the subject, wherein the monitoring comprises assessing the lupus condition of the subject at a plurality of time points, wherein the assessing is based at least on the lupus condition identified in (d) at each of the plurality of time points.

In some embodiments, a difference in the assessment of the lupus condition of the subject among the plurality of time points is indicative of one or more clinical indications selected from the group consisting of: (i) a diagnosis of the lupus condition of the subject, (ii) a prognosis of the lupus condition of the subject, and (iii) an efficacy or non-efficacy of a course of treatment for treating the lupus condition of the subject.

In some embodiments, the one or more reference LDG signatures are generated by: assaying a biological sample of one or more patients having one or more disease symptoms or being treated with one or more drugs to generate a reference dataset comprising gene expression data; and processing the reference dataset at each of the plurality of genes to determine quantitative measures of each of the plurality of genes.

In some embodiments, the one or more disease symptoms are selected from the group consisting of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance.

In some embodiments, the one or more drugs are selected from the group consisting of: antimalarials, corticosteroids, immunosuppressants, and nonsteroidal anti-inflammatory drugs (NSAIDs).

In another aspect, the present disclosure provides a computer system for identifying a lupus condition of a subject, comprising: a database that is configured to store a dataset comprising gene expression data, wherein the gene expression data is obtained by assaying a biological sample of the subject; and one or more computer processors operatively coupled to the database, wherein the one or more computer processors are individually or collectively programmed to: (i) process the dataset at each of a plurality of genes to determine quantitative measures of each of the plurality of genes, wherein the plurality of genes comprises low-density granulocyte (LDG)-associated genes, thereby producing an LDG signature of the biological sample of the subject; (ii) compare the LDG signature with one or more reference LDG signatures, wherein the comparing comprises, for at least one of the plurality of genes, comparing the quantitative measure of the gene of the LDG signature with corresponding quantitative measures of the gene of the one or more reference LDG signatures; and (iii) based at least in part on the comparison in (ii), identify the lupus condition of the subject.

In some embodiments, computer system further comprises an electronic display operatively coupled to the one or more computer processors, wherein the electronic display comprises a graphical user interface that is configured to display the report.

In another aspect, the present disclosure provides a non-transitory computer readable medium comprising machine-executable code that, upon execution by one or more computer processors, implements a method for identifying a lupus condition of a subject, the method comprising: (a) assaying a biological sample of the subject to generate a dataset comprising gene expression data; (b) processing the dataset at each of a plurality of genes to determine quantitative measures of each of the plurality of genes, wherein the plurality of genes comprises low-density granulocyte (LDG)-associated genes, thereby producing an LDG signature of the biological sample of the subject; (c) comparing the LDG signature with one or more reference LDG signatures, wherein the comparing comprises, for at least one of the plurality of genes, comparing the quantitative measure of the gene of the LDG signature with corresponding quantitative measures of the gene of the one or more reference LDG signatures; (d) based at least in part on the comparison in (c), identifying the lupus condition of the subject.

### Primary Immunodeficiency (PID) Profiling of Lupus Conditions

In another aspect, the present disclosure provides a method for identifying a lupus condition of a subject, comprising: (a) assaying a biological sample of the subject to generate a dataset comprising gene expression data; (b) processing the dataset at each of a plurality of genes to determine quantitative measures of each of the plurality of genes, wherein the plurality of genes comprises primary immunodeficiency (PID)-associated genes, thereby producing a PID signature of the biological sample of the subject; (c) processing the PID signature with one or more reference PID signatures, wherein the processing comprises, for at least one of the plurality of genes, comparing the quantitative measure of the gene of the PID signature with corresponding quantitative measures of the gene of the one or more reference PID signatures; (d) based at least in part on the comparison in (c), identifying the lupus condition of the subject.

In some embodiments, the lupus condition is selected from the group consisting of: systemic lupus erythematosus (SLE), discoid lupus erythematosus (DLE), and lupus nephritis (LN). In some embodiments, the biological sample is selected from the group consisting of: a whole blood (WB) sample, a PBMC sample, a tissue sample, and a cell sample. In some embodiments, the tissue sample is selected from the group consisting of: skin tissue, synovium tissue, kidney tissue, and bone marrow tissue. In some embodiments, the kidney tissue is selected from the group consisting of: glomerulus (Glom) and tubulointerstitium (TI). In some embodiments, the cell sample is selected from the group consisting of: myelocytes (MY), promyelocytes (PM), polymorphonuclear neutrophils (PMN), peripheral blood mononuclear cells (PBMC), and hematopoietic stem cells.

In some embodiments, the method further comprises enriching or purifying a whole blood sample of the subject to obtain the cell sample. In some embodiments, assaying the biological sample comprises (i) using a microarray to generate the dataset comprising the gene expression data, (ii) sequencing the biological sample to generate the dataset comprising the gene expression data, or (iii) performing quantitative polymerase chain reaction (qPCR) of the biological sample to generate the dataset comprising the gene expression data.

In some embodiments, the plurality of genes comprises PID-associated genes selected from the genes listed in Table 47. In some embodiments, the plurality of genes comprises at least 5 PID-associated genes selected from the genes listed in Table 47. In some embodiments, the plurality of genes comprises at least 10 PID-associated genes selected from the genes listed in Table 47. In some embodiments, the plurality of genes comprises at least 25 PID-associated genes selected from the genes listed in Table 47. In some embodiments, the plurality of genes comprises at least 50 PID-associated genes selected from the genes listed in Table 47. In some embodiments, the plurality of genes comprises at least 100 PID-associated genes selected from the genes listed in Table 47.

In some embodiments, the quantitative measures of each of the plurality of genes comprise enrichment scores of each of the plurality of genes. In some embodiments, the enrichment scores of each of the plurality of genes comprise gene set variation analysis (GSVA) enrichment scores of each of the plurality of genes. In some embodiments, (c) further comprises, for the at least one of the plurality of genes, determining a difference between the quantitative measure of the gene of the PID signature with the corresponding quantitative measures of the gene of the one or more reference PID signatures. In some embodiments, (d) further comprises identifying the lupus condition of the subject when the difference satisfies a pre-determined criterion.

In some embodiments, (c) further comprises, for the at least one of the plurality of genes, determining a Z-score of the quantitative measure of the gene of the PID signature relative to the corresponding quantitative measures of the gene of the one or more reference PID signatures. In some embodiments, (d) further comprises identifying the lupus condition of the subject when the Z-score satisfies a pre-determined criterion. In some embodiments, (d) further comprises identifying the lupus condition of the subject when the Z-score is at least about 3, and identifying an absence of the lupus condition of the subject when the Z-score is less than about 3. In some embodiments, (d) further comprises identifying the lupus condition of the subject when the Z-score is at least about 2.5, and identifying an absence of the lupus condition of the subject when the Z-score is less than about 2.5. In some embodiments, (d) further comprises identifying the lupus condition of the subject when the Z-score is at least about 2, and identifying an absence of the lupus condition of the subject when the Z-score is less than about 2. In some embodiments, (d) further comprises identifying the lupus condition of the subject when the Z-score is at least about 1.5, and identifying an absence of the lupus condition of the subject when the Z-score is less than about 1.5. In some embodiments, (d) further comprises identifying the lupus condition of the subject when the Z-score is at least about 1, and identifying an absence of the lupus condition of the subject when the Z-score is less than about 1. In some embodiments, (d) further comprises identifying the lupus condition of the subject when the Z-score is at least about 0.5, and identifying an absence of the lupus condition of the subject when the Z-score is less than about 0.5.

In some embodiments, the method further comprises identifying the lupus condition of the subject at a sensitivity of at least about 60%. In some embodiments, the method further comprises identifying the lupus condition of the subject at a sensitivity of at least about 65%. In some embodiments, the method further comprises identifying the lupus condition of the subject at a sensitivity of at least about 70%. In some embodiments, the method further comprises identifying the lupus condition of the subject at a sensitivity of at least about 75%. In some embodiments, the method further comprises identifying the lupus condition of the subject at a sensitivity of at least about 80%. In some embodiments, the method further comprises identifying the lupus condition of the subject at a sensitivity of at least about 85%. In some embodiments, the method further comprises identifying the lupus condition of the subject at a sensitivity of at least about 90%. In some embodiments, the method further comprises identifying the lupus condition of the subject at a sensitivity of at least about 95%. In some embodiments, the method further comprises identifying the lupus condition of the subject at a sensitivity of at least about 99%.

In some embodiments, the method further comprises identifying the lupus condition of the subject at a specificity of at least about 60%. In some embodiments, the method further comprises identifying the lupus condition of the subject at a specificity of at least about 65%. In some embodiments, the method further comprises identifying the lupus condition of the subject at a specificity of at least about 70%. In some embodiments, the method further comprises identifying the lupus condition of the subject at a specificity of at least about 75%. In some embodiments, the method further comprises identifying the lupus condition of the subject at a specificity of at least about 80%. In some embodiments, the method further comprises identifying the lupus condition of the subject at a specificity of at least about 85%. In some embodiments, the method further comprises identifying the lupus condition of the subject at a specificity of at least about 90%. In some embodiments, the method further comprises identifying the lupus condition of the subject at a specificity of at least about 95%. In some embodiments, the method further comprises identifying the lupus condition of the subject at a specificity of at least about 99%.

In some embodiments, the method further comprises identifying the lupus condition of the subject at a positive predictive value (PPV) of at least about 60%. In some embodiments, the method further comprises identifying the lupus condition of the subject at a positive predictive value (PPV) of at least about 65%. In some embodiments, the method further comprises identifying the lupus condition of the subject at a positive predictive value (PPV) of at least about 70%. In some embodiments, the method further comprises identifying the lupus condition of the subject at a positive predictive value (PPV) of at least about 75%. In some embodiments, the method further comprises identifying the lupus condition of the subject at a positive predictive value (PPV) of at least about 80%. In some embodiments, the method further comprises identifying the lupus condition of the subject at a positive predictive value (PPV) of at least about 85%. In some embodiments, the method further comprises identifying the lupus condition of the subject at a positive predictive value (PPV) of at least about 90%. In some embodiments, the method further comprises identifying the lupus condition of the subject at a positive predictive value (PPV) of at least about 95%. In some embodiments, the method further comprises identifying the lupus condition of the subject at a positive predictive value (PPV) of at least about 99%.

In some embodiments, the method further comprises identifying the lupus condition of the subject at a negative predictive value (NPV) of at least about 60%. In some embodiments, the method further comprises identifying the lupus condition of the subject at a negative predictive value (NPV) of at least about 65%. In some embodiments, the method further comprises identifying the lupus condition of the subject at a negative predictive value (NPV) of at least about 70%. In some embodiments, the method further comprises identifying the lupus condition of the subject at a negative predictive value (NPV) of at least about 75%. In some embodiments, the method further comprises identifying the lupus condition of the subject at a negative predictive value (NPV) of at least about 80%. In some embodiments, the method further comprises identifying the lupus condition of the subject at a negative predictive value (NPV) of at least about 85%. In some embodiments, the method further comprises identifying the lupus condition of the subject at a negative predictive value (NPV) of at least about 90%. In some embodiments, the method further comprises identifying the lupus condition of the subject at a negative predictive value (NPV) of at least about 95%. In some embodiments, the method further comprises identifying the lupus condition of the subject at a negative predictive value (NPV) of at least about 99%.

In some embodiments, the method further comprises identifying the lupus condition of the subject with an Area Under Curve (AUC) of at least about 0.60. In some embodiments, the method further comprises identifying the lupus condition of the subject with an Area Under Curve (AUC) of at least about 0.65. In some embodiments, the method further comprises identifying the lupus condition of the subject with an Area Under Curve (AUC) of at least about 0.70. In some embodiments, the method further comprises identifying the lupus condition of the subject with an Area Under Curve (AUC) of at least about 0.75. In some embodiments, the method further comprises identifying the lupus condition of the subject with an Area Under Curve (AUC) of at least about 0.80. In some embodiments, the method further comprises identifying the lupus condition of the subject with an Area Under Curve (AUC) of at least about 0.85. In some embodiments, the method further comprises identifying the lupus condition of the subject with an Area Under Curve (AUC) of at least about 0.90. In some embodiments, the method further comprises identifying the lupus condition of the subject with an Area Under Curve (AUC) of at least about 0.95. In some embodiments, the method further comprises identifying the lupus condition of the subject with an Area Under Curve (AUC) of at least about 0.99.

In some embodiments, (d) further comprises identifying the lupus condition of the subject based at least in part on a SLEDAI score of the subject. In some embodiments, the subject is asymptomatic for one or more lupus conditions selected from the group consisting of: systemic lupus erythematosus (SLE), discoid lupus erythematosus (DLE), and lupus nephritis (LN).

In some embodiments, the method further comprises applying a trained algorithm to the PID signature to identify the lupus condition of the subject. In some embodiments, the trained algorithm is trained using a first set of independent training samples associated with a presence of the lupus condition and a second set of independent training samples associated with an absence of the lupus condition. In some embodiments, the method further comprises using the trained algorithm to process a set of clinical health data of the subject to identify the lupus condition. In some embodiments, the trained algorithm comprises a supervised machine learning algorithm. In some embodiments, the supervised machine learning algorithm comprises a deep learning algorithm, a support vector machine (SVM), a neural network, or a Random Forest.

In some embodiments, (a) comprises (i) subjecting the biological sample to conditions that are sufficient to isolate, enrich, or extract a plurality of nucleic acid molecules; and (ii) analyzing the plurality of nucleic acid molecules to generate the dataset comprising the gene expression data.

In some embodiments, the method further comprises using probes configured to selectively enrich the plurality of nucleic acid molecules corresponding to a panel of one or more genomic loci. In some embodiments, the probes are nucleic acid primers. In some embodiments, the probes have sequence complementarity with nucleic acid sequences of the panel of the one or more genomic loci. In some embodiments, the panel of the one or more genomic loci comprises genomic loci corresponding to the plurality of genes. In some embodiments, the panel of said one or more genomic loci comprises at least 5 distinct genomic loci. In some embodiments, the panel of said one or more genomic loci comprises at least 10 distinct genomic loci. In some embodiments, the panel of said one or more genomic loci comprises at least 25 distinct genomic loci. In some embodiments, the panel of said one or more genomic loci comprises at least 50 distinct genomic loci. In some embodiments, the panel of said one or more genomic loci comprises at least 100 distinct genomic loci. In some embodiments, the panel of said one or more genomic loci comprises at least 150 distinct genomic loci.

In some embodiments, the method further comprises (e) assaying a second biological sample of the subject to generate a second dataset comprising gene expression data; (f) processing the second dataset at each of the plurality of genes to determine second quantitative measures of each of the plurality of genes, thereby producing a second PID signature of the second biological sample of the subject; (g) processing the second PID signature with one or more reference PID signatures, wherein the processing comprises, for at least one of the plurality of genes, comparing the quantitative measure of the gene of the second PID signature with corresponding quantitative measures of the gene of the one or more reference PID signatures; and (h) based at least in part on the comparison in (g), identifying the lupus condition of the subject.

In some embodiments, the biological sample and the second biological sample comprise two different sample types selected from the group consisting of: a whole blood (WB) sample, a PBMC sample, a skin tissue sample, a synovium tissue sample, a kidney tissue sample comprising glomerulus (Glom), a kidney tissue sample comprising tubulointerstitium (TI), a bone marrow tissue, a myelocyte (MY) cell sample, a promyelocyte (PM) cell sample, a polymorphonuclear neutrophils (PMN) sample, and a hematopoietic stem cell sample.

In some embodiments, the method further comprises determining a likelihood of the identification of the lupus condition of the subject. In some embodiments, the method further comprises providing a therapeutic intervention for the lupus condition of the subject.

In some embodiments, the method further comprises monitoring the lupus condition of the subject, wherein the monitoring comprises assessing the lupus condition of the subject at a plurality of time points, wherein the assessing is based at least on the lupus condition identified in (d) at each of the plurality of time points.

In some embodiments, a difference in the assessment of the lupus condition of the subject among the plurality of time points is indicative of one or more clinical indications selected from the group consisting of: (i) a diagnosis of the lupus condition of the subject, (ii) a prognosis of the lupus condition of the subject, and (iii) an efficacy or non-efficacy of a course of treatment for treating the lupus condition of the subject.

In some embodiments, the one or more reference PID signatures are generated by: assaying a biological sample of one or more patients having one or more disease symptoms or being treated with one or more drugs to generate a reference dataset comprising gene expression data; and processing the reference dataset at each of the plurality of genes to determine quantitative measures of each of the plurality of genes.

In some embodiments, the one or more disease symptoms are selected from the group consisting of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance.

In some embodiments, the one or more drugs are selected from the group consisting of: antimalarials, corticosteroids, immunosuppressants, and nonsteroidal anti-inflammatory drugs (NSAIDs).

In another aspect, the present disclosure provides a computer system for identifying a lupus condition of a subject, comprising: a database that is configured to store a dataset comprising gene expression data, wherein the gene expression data is obtained by assaying a biological sample of the subject; and one or more computer processors operatively coupled to the database, wherein the one or more computer processors are individually or collectively programmed to: (i) process the dataset at each of a plurality of genes to determine quantitative measures of each of the plurality of genes, wherein the plurality of genes comprises primary immunodeficiency (PID)-associated genes, thereby producing a PID signature of the biological sample of the subject; (ii) process the PID signature with one or more reference PID signatures, wherein the processing comprises, for at least one of the plurality of genes, comparing the quantitative measure of the gene of the PID signature with corresponding quantitative measures of the gene of the one or more reference PID signatures; and (iii) based at least in part on the comparison in (ii), identify the lupus condition of the subject.

In some embodiments, computer system further comprises an electronic display operatively coupled to the one or more computer processors, wherein the electronic display comprises a graphical user interface that is configured to display the report.

In another aspect, the present disclosure provides a non-transitory computer readable medium comprising machine-executable code that, upon execution by one or more computer processors, implements a method for identifying a lupus condition of a subject, the method comprising: (a) obtaining a dataset comprising gene expression data, wherein the gene expression data is generated by assaying a biological sample of the subject; (b) processing the dataset at each of a plurality of genes to determine quantitative measures of each of the plurality of genes, wherein the plurality of genes comprises primary immunodeficiency (PID)-associated genes, thereby producing a PID signature of the biological sample of the subject; (c) processing the PID signature with one or more reference PID signatures, wherein the processing comprises, for at least one of the plurality of genes, comparing the quantitative measure of the gene of the PID signature with corresponding quantitative measures of the gene of the one or more reference PID signatures; (d) based at least in part on the comparison in (c), identifying the lupus condition of the subject.

### Biological Data Analysis

In another aspect, the present disclosure provides a computer-implemented method for assessing a condition of a subject, comprising: (a) receiving a dataset of a biological sample of the subject; (b) selecting one or more data analysis tools, wherein the one or more data analysis tools comprise an analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool, or a combination thereof; (c) processing the dataset using the one or more data analysis tools to generate a data signature of the biological sample of the subject; and (d) based at least in part on the data signature generated in (c), assessing the condition of the subject.

In some embodiments, the dataset comprises mRNA gene expression or transcriptome data, DNA genomic data, proteomic data, metabolomic data, or a combination thereof. In some embodiments, the biological sample is selected from the group consisting of: a whole blood (WB) sample, a PBMC sample, a tissue sample, and a cell sample. In some embodiments, assessing the condition of the subject comprises identifying a disease or disorder of the subject.

In some embodiments, the method further comprises identifying a disease or disorder of the subject at a sensitivity or specificity of at least about 70%. In some embodiments, the method further comprises determining a likelihood of the identification of the disease or disorder of the subject. In some embodiments, the method further comprises providing a therapeutic intervention for the disease or disorder of the subject. In some embodiments, the method further comprises monitoring the disease or disorder of the subject, wherein the monitoring comprises assessing the disease or disorder of the subject at a plurality of time points, wherein the assessing is based at least on the disease or disorder identified at each of the plurality of time points.

In some embodiments, selecting the one or more data analysis tools comprises receiving a user selection of the one or more data analysis tools. In some embodiments, selecting the one or more data analysis tools is automatically performed by the computer without receiving a user selection of the one or more data analysis tools.

In another aspect, the present disclosure provides a computer system for assessing a condition of a subject, comprising: a database that is configured to store a dataset of a biological sample of the subject; and one or more computer processors operatively coupled to the database, wherein the one or more computer processors are individually or collectively programmed to: (i) select one or more data analysis tools, wherein the one or more data analysis tools comprise an analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool; (ii) process the dataset using the one or more data analysis tools to generate a data signature of the biological sample of the subject; and (iii) based at least in part on the data signature generated in (ii), assess the condition of the subject.

In another aspect, the present disclosure provides a non-transitory computer readable medium comprising machine-executable code that, upon execution by one or more computer processors, implements a method for assessing a condition of a subject, the method comprising: (a) receiving a dataset of a biological sample of the subject; (b) selecting one or more data analysis tools, wherein the one or more data analysis tools comprise an analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool; (c) processing the dataset using the one or more data analysis tools to generate a data signature of the biological sample of the subject; and (d) based at least in part on the data signature generated in (c), assessing the condition of the subject. In any embodiment described herein, the one or more data analysis tools can be a plurality of data analysis tools each independently selected from a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool.

Analysis of Single Nucleotide Polymorphisms (SNPs) Associated with Lupus

In another aspect, the present disclosure provides a computer-implemented method for assessing an SLE condition of a subject, comprising: (a) receiving a dataset of a biological sample of the subject, wherein the dataset comprises quantitative measures of gene expression at each a plurality of SLE-associated genomic loci, wherein the plurality of SLE-associated genomic loci comprises (i) one or more AA-specific single nucleotide polymorphisms (SNPs) if the subject has an African-Ancestry (AA), or (ii) one or more EA-specific SNPs if the subject has a European-Ancestry (EA); (b) processing the dataset to identify one or more differentially expressed (DE) genomic loci among the plurality of SLE-associated genomic loci; and (c) based at least in part on the one or more DE genomic loci identified in (b) and whether the subject has an African-Ancestry (AA) or a European-Ancestry (EA), assessing the SLE condition of the subject.

In another aspect, the present disclosure provides a computer-implemented method for assessing an SLE condition of a subject, comprising: (a) receiving a dataset of a biological sample of the subject, wherein the dataset comprises quantitative measures of gene expression at each a plurality of systemic lupus erythematosus (SLE)-associated genomic loci, wherein the plurality of SLE-associated genomic loci comprises one or more African-Ancestry (AA)-specific single nucleotide polymorphisms (SNPs); (b) processing the dataset to identify one or more differentially expressed (DE) genomic loci among the plurality of SLE-associated genomic loci; and (c) based at least in part on the one or more DE genomic loci identified in (b) and whether the subject has an African-Ancestry (AA), assessing the SLE condition of the subject.

In another aspect, the present disclosure provides a computer-implemented method for assessing an SLE condition of a subject, comprising: (a) receiving a dataset of a biological sample of the subject, wherein the dataset comprises quantitative measures of gene expression at each a plurality of systemic lupus erythematosus (SLE)-associated genomic loci, wherein the plurality of SLE-associated genomic loci comprises one or more European-Ancestry (EA)-specific single nucleotide polymorphisms (SNPs); (b) processing the dataset to identify one or more differentially expressed (DE) genomic loci among the plurality of SLE-associated genomic loci; and (c) based at least in part on the one or more DE genomic loci identified in (b) and whether the subject has a European-Ancestry (EA), assessing the SLE condition of the subject.

In some embodiments, the dataset comprises RNA gene expression or transcriptome data, DNA genomic data, or a combination thereof. In some embodiments, the biological sample is selected from the group consisting of: a whole blood (WB) sample, a PBMC sample, a tissue sample, and a cell sample. In some embodiments, assessing the SLE condition of the subject comprises determining a diagnosis of the SLE condition, a prognosis of the SLE condition, a susceptibility of the SLE condition, a treatment for the SLE condition, or an efficacy or non-efficacy of a treatment for the SLE condition.

In some embodiments, the method further comprises determining a diagnosis of the SLE condition with a sensitivity of at least about 70%. In some embodiments, the method further comprises determining a diagnosis of the SLE condition with a specificity of at least about 70%. In some embodiments, the method further comprises determining a diagnosis of the SLE condition with a positive predictive value of at least about 70%. In some embodiments, the method further comprises determining a diagnosis of the SLE condition with a negative predictive value of at least about 70%. In some embodiments, the method further comprises determining a diagnosis of the SLE condition with an Area Under Curve (AUC) of at least about 70%. In some embodiments, the method further comprises determining a likelihood of the diagnosis of the SLE condition of the subject.

In some embodiments, the method further comprises generating a plurality of drug candidates for the SLE condition of the subject. In some embodiments, the method further comprises evaluating or predicting a relative efficacy of the plurality of drug candidates for the SLE condition of the subject. In some embodiments, the method further comprises providing a therapeutic intervention comprising one or more of the plurality of drug candidates for the SLE condition of the subject.

In some embodiments, the method further comprises selecting a treatment for the SLE condition of the subject, the treatment comprising an AA-specific drug. In some embodiments, the AA-specific drug is selected from the group consisting of: an HDAC inhibitor, a retinoid, a IRAK4-targeted drug, and a CTLA4-targeted drug. In some embodiments, the method further comprises selecting a treatment for the SLE condition of the subject, the treatment comprising an EA-specific drug. In some embodiments, the EA-specific drug is selected from the group consisting of: hydroxychloroquine, a CD40LG-targeted drug, a CXCR1-targeted drug, and a CXCR2-targeted drug. In some embodiments, the method further comprises selecting a treatment for the SLE condition of the subject, the treatment comprising a drug targeting E-Genes or pathways shared by EA and AA. In some embodiments, the drug targeting E-Genes or pathways shared by EA and AA is selected from the group consisting of: ibrutinib, ruxolitinib, and ustekinumab.

In some embodiments, the method further comprises monitoring the SLE condition of the subject, wherein the monitoring comprises assessing the SLE condition of the subject at each of a plurality of time points, and processing the plurality of assessments of the SLE condition of the subject at each of the plurality of time points.

In some embodiments, the one or more EA-specific SNPs comprise one or more SNPs of genes selected from the group listed in Table 56. In some embodiments, the one or more AA-specific SNPs comprise one or more SNPs of genes selected from the group listed in Table 57. In some embodiments, the plurality of SLE-associated genomic loci comprises one or more shared SNPs, wherein the one or more shared SNPs are common to both EA and AA. In some embodiments, the one or more shared SNPs comprise one or more SNPs of genes selected from the group listed in Table 58.

In another aspect, the present disclosure provides a computer system for assessing an SLE condition of a subject, comprising: a database that is configured to store an African-Ancestry (AA) status of the subject, a European-Ancestry (EA) status of the subject, and a dataset of a biological sample of the subject, wherein the dataset comprises quantitative measures of gene expression at each a plurality of SLE-associated genomic loci, wherein the plurality of SLE-associated genomic loci comprises (i) one or more AA-specific single nucleotide polymorphisms (SNPs) if the subject has an African-Ancestry (AA), or (ii) one or more EA-specific SNPs if the subject has a European-Ancestry (EA); and one or more computer processors operatively coupled to the database, wherein the one or more computer processors are individually or collectively programmed to: (i) process the dataset to identify one or more differentially expressed (DE) genomic loci among the plurality of SLE-associated genomic loci; and (ii) based at least in part on the one or more DE genomic loci identified in (ii), the AA status of the subject, and the EA status of the subject, assessing the SLE condition of the subject.

In another aspect, the present disclosure provides a computer system for assessing an SLE condition of a subject, comprising: a database that is configured to store an African-Ancestry (AA) status of the subject and a dataset of a biological sample of the subject, wherein the dataset comprises quantitative measures of gene expression at each a plurality of systemic lupus erythematosus (SLE)-associated genomic loci, wherein the plurality of SLE-associated genomic loci comprises one or more African-Ancestry (AA)-specific single nucleotide polymorphisms (SNPs); and one or more computer processors operatively coupled to the database, wherein the one or more computer processors are individually or collectively programmed to: (i) process the dataset to identify one or more differentially expressed (DE) genomic loci among the plurality of SLE-associated genomic loci; and (ii) based at least in part on the one or more DE genomic loci identified in (ii) and the AA status of the subject, assessing the SLE condition of the subject.

In some embodiments, In another aspect, the present disclosure provides a computer system for assessing an SLE condition of a subject, comprising: a database that is configured to store a European-Ancestry (EA) status of the subject and a dataset of a biological sample of the subject, wherein the dataset comprises quantitative measures of gene expression at each a plurality of systemic lupus erythematosus (SLE)-associated genomic loci, wherein the plurality of SLE-associated genomic loci comprises one or more European-Ancestry (EA)-specific single nucleotide polymorphisms (SNPs); and one or more computer processors operatively coupled to the database, wherein the one or more computer processors are individually or collectively programmed to: (i) process the dataset to identify one or more differentially expressed (DE) genomic loci among the plurality of SLE-associated genomic loci; and (ii) based at least in part on the one or more DE genomic loci identified in (i) and the EA status of the subject, assess the SLE condition of the subject.

In another aspect, the present disclosure provides a non-transitory computer readable medium comprising machine-executable code that, upon execution by one or more computer processors, implements a method for assessing an SLE condition of a subject, comprising: (a) receiving a dataset of a biological sample of the subject, wherein the dataset comprises quantitative measures of gene expression at each a plurality of SLE-associated genomic loci, wherein the plurality of SLE-associated genomic loci comprises (i) one or more AA-specific single nucleotide polymorphisms (SNPs) if the subject has an African-Ancestry (AA), or (ii) one or more EA-specific SNPs if the subject has a European-Ancestry (EA); (b) processing the dataset to identify one or more differentially expressed (DE) genomic loci among the plurality of SLE-associated genomic loci; and (c) based at least in part on the one or more DE genomic loci identified in (b) and whether the subject has an African-Ancestry (AA) or a European-Ancestry (EA), assessing the SLE condition of the subject.

In another aspect, the present disclosure provides a non-transitory computer readable medium comprising machine-executable code that, upon execution by one or more computer processors, implements a method for assessing an SLE condition of a subject, comprising: (a) receiving a dataset of a biological sample of the subject, wherein the dataset comprises quantitative measures of gene expression at each a plurality of systemic lupus erythematosus (SLE)-associated genomic loci, wherein the plurality of SLE-associated genomic loci comprises one or more African-Ancestry (AA)-specific single nucleotide polymorphisms (SNPs); (b) processing the dataset to identify one or more differentially expressed (DE) genomic loci among the plurality of SLE-associated genomic loci; and (c) based at least in part on the one or more DE genomic loci identified in (b) and whether the subject has an African-Ancestry (AA), assessing the SLE condition of the subject.

In another aspect, the present disclosure provides a non-transitory computer readable medium comprising machine-executable code that, upon execution by one or more computer processors, implements a method for assessing an SLE condition of a subject, comprising: (a) receiving a dataset of a biological sample of the subject, wherein the dataset comprises quantitative measures of gene expression at each a plurality of systemic lupus erythematosus (SLE)-associated genomic loci, wherein the plurality of SLE-associated genomic loci comprises one or more European-Ancestry (EA)-specific single nucleotide polymorphisms (SNPs); (b) processing the dataset to identify one or more differentially expressed (DE) genomic loci among the plurality of SLE-associated genomic loci; and (c) based at least in part on the one or more DE genomic loci identified in (b) and whether the subject has a European-Ancestry (EA) assessing the SLE condition of the subject.

Analysis of Single Nucleotide Polymorphisms (SNPs) Associated with Lupus

In another aspect, the present disclosure provides a method for identifying an autoimmune disease drug target, the method comprising: (a) treating an autoimmune disease animal model with a drug configured to inhibit a drug target of the autoimmune disease, thereby producing a treated animal model; (b) assaying an animal biological sample of the treated animal model to obtain gene expression data of the treated animal model; (c) processing the gene expression data to obtain an animal gene signature, wherein the animal gene signature comprises quantitative measures of a first set of genomic loci associated with autoimmune disease pathways of the autoimmune disease animal model; (d) obtaining a set of human gene signatures, wherein the set of human gene signatures comprises quantitative measures of a second set of genomic loci associated with up-regulation or down-regulation of human autoimmune disease pathways in human patients having active autoimmune disease, and wherein the set of human gene signatures is generated by assaying human biological samples from one or more human patients having the autoimmune disease to obtain gene expression data; (e) processing the animal gene signature with the set of human gene signatures to identify (i) an animal genomic locus from among the first set of genomic loci, and (ii) a human genomic locus from among the second set of genomic loci that is associated with up-regulation or down-regulation of one or more human autoimmune disease pathways, wherein the animal genomic locus and the human genomic locus are orthologous and share similarity in expression patterns and function; and (f) identifying the drug target as the autoimmune disease drug target when the quantitative measure of the animal genomic locus of the animal gene signature is indicative of up-regulation or down-regulation of an autoimmune disease pathway of the autoimmune disease animal model.

In some embodiments, the autoimmune disease animal model is selected from: a mouse model, a rat model, a cat model, a dog model, a rabbit model, a guinea pig model, a hamster model, a pig model, a horse model, and a primate model. In some embodiments, the autoimmune disease animal model comprises a mouse model. In some embodiments, the autoimmune disease comprises lupus. In some embodiments, the lupus comprises systemic lupus erythematosus (SLE) or discoid lupus erythematosus (DLE). In some embodiments, the drug target is HDAC6. In some embodiments, the drug target is HDAC6 or a portion thereof. In some embodiments, the drug is an HDAC6 inhibitor. In some embodiments, the HDAC6 inhibitor is ACY-738. In some embodiments, the animal biological sample or the human biological samples comprise one or more of: a bodily fluid sample, a blood sample, a cell sample, and a tissue sample. In some embodiments, the one or more human autoimmune disease pathways are selected from the pathways listed in Table 61, Table 62, Table 63, and Table 64. In some embodiments, the human genomic locus that is associated with up-regulation or down-regulation of the one or more human autoimmune disease pathways is selected from the genes listed in Table 59, Table 60, Table 61, Table 62, Table 65, Table 66, and Table 67. In some embodiments, the autoimmune disease pathways of the autoimmune disease animal model are selected from the pathways listed in Table 61, Table 62, Table 63, and Table 64. In some embodiments, the animal genomic locus is selected from the genes listed in Table 59, Table 60, Table 61, Table 62, Table 65, Table 66, and Table 67. In some embodiments, (e) comprises identifying (i) a plurality of animal genomic loci from among the first set of genomic loci, and (ii) a plurality of human genomic loci from among the second set of genomic loci that is associated with up-regulation or down-regulation of a plurality of human autoimmune disease pathways, wherein plurality of animal genomic loci and the plurality of human genomic loci are pairwise orthologous and share similarities in expression patterns and function; and (f) comprises identifying the drug target as the autoimmune disease drug target when the quantitative measures of the plurality of animal genomic loci of the animal gene signature are indicative of up-regulation or down-regulation of a plurality of autoimmune disease pathways of the autoimmune disease animal model. In some embodiments, the plurality of human autoimmune disease pathways comprises between 2 and 5 different human autoimmune disease pathways. In some embodiments, the plurality of human autoimmune disease pathways comprises 1, 2, 3, 4, 5, 6, 7, 8, 9, 10, 11, 12, 13, 14, 15, 16, 17, 18, 19, 20, about 25, about 30, about 35, about 40, about 45, about 50, about 60, about 70, about 80, about 90, about 100, or more than about 100 different human autoimmune disease pathways. In some embodiments, the autoimmune disease pathways of the autoimmune disease animal model comprises 1, 2, 3, 4, 5, 6, 7, 8, 9, 10, 11, 12, 13, 14, 15, 16, 17, 18, 19, 20, about 25, about 30, about 35, about 40, about 45, about 50, about 60, about 70, about 80, about 90, about 100, or more than about 100 different autoimmune disease pathways. In some embodiments, the method further comprises determining the up-regulation or down-regulation of the autoimmune disease pathway of the autoimmune disease animal model based on determining a difference between the quantitative measure of the animal genomic locus of the animal gene signature and a reference quantitative measure of the animal genomic locus. In some embodiments, the method further comprises obtaining the reference quantitative measure of the animal genomic locus by, prior to (a), assaying an animal biological sample of the autoimmune disease animal model.

In another aspect, the present disclosure provides a computer-implemented method for identifying an autoimmune disease drug target, the method comprising: (a) obtaining gene expression data generated by assaying an animal biological sample of a treated animal model, wherein the treated animal model is obtained by treating an autoimmune disease animal model with a drug configured to inhibit a drug target of the autoimmune disease; (b) processing the gene expression data to obtain an animal gene signature, wherein the animal gene signature comprises quantitative measures of a first set of genomic loci associated with autoimmune disease pathways of the autoimmune disease animal model; (c) obtaining a set of human gene signatures, wherein the set of human gene signatures comprises quantitative measures of a second set of genomic loci associated with up-regulation or down-regulation of human autoimmune disease pathways in human patients having active autoimmune disease, and wherein the set of human gene signatures is generated by assaying human biological samples from one or more human patients having the autoimmune disease to obtain gene expression data; (d) processing the animal gene signature with the set of human gene signatures to identify (i) an animal genomic locus from among the first set of genomic loci, and (ii) a human genomic locus from among the second set of genomic loci that is associated with up-regulation or down-regulation of one or more human autoimmune disease pathways, wherein the animal genomic locus and the human genomic locus are orthologous and share similarity in expression patterns and function; and (e) identifying the drug target as the autoimmune disease drug target when the quantitative measure of the animal genomic locus of the animal gene signature is indicative of up-regulation or down-regulation of an autoimmune disease pathway of the autoimmune disease animal model.

In some embodiments, the autoimmune disease animal model is selected from: a mouse model, a rat model, a cat model, a dog model, a rabbit model, a guinea pig model, a hamster model, a pig model, a horse model, and a primate model. In some embodiments, the autoimmune disease animal model comprises a mouse model. In some embodiments, the autoimmune disease comprises lupus. In some embodiments, the lupus comprises systemic lupus erythematosus (SLE) or discoid lupus erythematosus (DLE). In some embodiments, the drug target is HDAC6. In some embodiments, the drug target is HDAC6 or a portion thereof. In some embodiments, the drug is an HDAC6 inhibitor. In some embodiments, the HDAC6 inhibitor is ACY-738. In some embodiments, the animal biological sample or the human biological samples comprise one or more of: a bodily fluid sample, a blood sample, a cell sample, and a tissue sample. In some embodiments, the one or more human autoimmune disease pathways are selected from the pathways listed in Table 61, Table 62, Table 63, and Table 64. In some embodiments, the human genomic locus that is associated with up-regulation or down-regulation of the one or more human autoimmune disease pathways is selected from the genes listed in Table 59, Table 60, Table 61, Table 62, Table 65, Table 66, and Table 67. In some embodiments, the autoimmune disease pathways of the autoimmune disease animal model are selected from the pathways listed in Table 61, Table 62, Table 63, and Table 64. In some embodiments, the animal genomic locus is selected from the genes listed in Table 59, Table 60, Table 61, Table 62, Table 65, Table 66, and Table 67. In some embodiments, (d) comprises identifying (i) a plurality of animal genomic loci from among the first set of genomic loci, and (ii) a plurality of human genomic loci from among the second set of genomic loci that is associated with up-regulation or down-regulation of a plurality of human autoimmune disease pathways, wherein plurality of animal genomic loci and the plurality of human genomic loci are pairwise orthologous and share similarities in expression patterns and function; and (e) comprises identifying the drug target as the autoimmune disease drug target when the quantitative measures of the plurality of animal genomic loci of the animal gene signature are indicative of up-regulation or down-regulation of a plurality of autoimmune disease pathways of the autoimmune disease animal model. In some embodiments, the plurality of human autoimmune disease pathways comprises between 2 and 5 different human autoimmune disease pathways. In some embodiments, the plurality of human autoimmune disease pathways comprises 1, 2, 3, 4, 5, 6, 7, 8, 9, 10, 11, 12, 13, 14, 15, 16, 17, 18, 19, 20, about 25, about 30, about 35, about 40, about 45, about 50, about 60, about 70, about 80, about 90, about 100, or more than about 100 different human autoimmune disease pathways. In some embodiments, the autoimmune disease pathways of the autoimmune disease animal model comprises 1, 2, 3, 4, 5, 6, 7, 8, 9, 10, 11, 12, 13, 14, 15, 16, 17, 18, 19, 20, about 25, about 30, about 35, about 40, about 45, about 50, about 60, about 70, about 80, about 90, about 100, or more than about 100 different autoimmune disease pathways. In some embodiments, the method further comprises determining the up-regulation or down-regulation of the autoimmune disease pathway of the autoimmune disease animal model based on determining a difference between the quantitative measure of the animal genomic locus of the animal gene signature and a reference quantitative measure of the animal genomic locus. In some embodiments, the method further comprises obtaining the reference quantitative measure of the animal genomic locus by, prior to (a), assaying an animal biological sample of the autoimmune disease animal model.

In another aspect, the present disclosure provides a computer system for identifying an autoimmune disease drug target, comprising: a database that is configured to store gene expression data generated by assaying an animal biological sample of a treated animal model, wherein the treated animal model is obtained by treating an autoimmune disease animal model with a drug configured to inhibit a drug target of the autoimmune disease; and one or more computer processors operatively coupled to the database, wherein the one or more computer processors are individually or collectively programmed to: (i) process the transcriptomic data to obtain an animal gene signature, wherein the animal gene signature comprises quantitative measures of a first set of genomic loci associated with autoimmune disease pathways of the autoimmune disease animal model; (ii) obtain a set of human gene signatures, wherein the set of human gene signatures comprises quantitative measures of a second set of genomic loci associated with up-regulation or down-regulation of human autoimmune disease pathways in human patients having active autoimmune disease, and wherein the set of human gene signatures is generated by assaying human biological samples from one or more human patients having the autoimmune disease to obtain gene expression data; (iii) process the animal gene signature with the set of human gene signatures to identify (1) an animal genomic locus from among the first set of genomic loci, and (2) a human genomic locus from among the second set of genomic loci that is associated with up-regulation or down-regulation of one or more human autoimmune disease pathways, wherein the animal genomic locus and the human genomic locus are orthologous and share similarity in expression patterns and function; and (iv) identify the drug target as the autoimmune disease drug target when the quantitative measure of the animal genomic locus of the animal gene signature is indicative of up-regulation or down-regulation of an autoimmune disease pathway of the autoimmune disease animal model.

In some embodiments, the autoimmune disease animal model is selected from: a mouse model, a rat model, a cat model, a dog model, a rabbit model, a guinea pig model, a hamster model, a pig model, a horse model, and a primate model. In some embodiments, the autoimmune disease animal model comprises a mouse model. In some embodiments, the autoimmune disease comprises lupus. In some embodiments, the lupus comprises systemic lupus erythematosus (SLE) or discoid lupus erythematosus (DLE). In some embodiments, the drug target is HDAC6. In some embodiments, the drug target is HDAC6 or a portion thereof. In some embodiments, the drug is an HDAC6 inhibitor. In some embodiments, the HDAC6 inhibitor is ACY-738. In some embodiments, the animal biological sample or the human biological samples comprise one or more of: a bodily fluid sample, a blood sample, a cell sample, and a tissue sample. In some embodiments, the one or more human autoimmune disease pathways are selected from the pathways listed in Table 61, Table 62, Table 63, and Table 64. In some embodiments, the human genomic locus that is associated with up-regulation or down-regulation of the one or more human autoimmune disease pathways is selected from the genes listed in Table 59, Table 60, Table 61, Table 62, Table 65, Table 66, and Table 67. In some embodiments, the autoimmune disease pathways of the autoimmune disease animal model are selected from the pathways listed in Table 61, Table 62, Table 63, and Table 64. In some embodiments, the animal genomic locus is selected from the genes listed in Table 59, Table 60, Table 61, Table 62, Table 65, Table 66, and Table 67. In some embodiments, (iii) comprises identifying (1) a plurality of animal genomic loci from among the first set of genomic loci, and (2) a plurality of human genomic loci from among the second set of genomic loci that is associated with up-regulation or down-regulation of a plurality of human autoimmune disease pathways, wherein plurality of animal genomic loci and the plurality of human genomic loci are pairwise orthologous and share similarities in expression patterns and function; and (iv) comprises identifying the drug target as the autoimmune disease drug target when the quantitative measures of the plurality of animal genomic loci of the animal gene signature are indicative of up-regulation or down-regulation of a plurality of autoimmune disease pathways of the autoimmune disease animal model. In some embodiments, the plurality of human autoimmune disease pathways comprises between 2 and 5 different human autoimmune disease pathways. In some embodiments, the plurality of human autoimmune disease pathways comprises 1, 2, 3, 4, 5, 6, 7, 8, 9, 10, 11, 12, 13, 14, 15, 16, 17, 18, 19, 20, about 25, about 30, about 35, about 40, about 45, about 50, about 60, about 70, about 80, about 90, about 100, or more than about 100 different human autoimmune disease pathways. In some embodiments, the autoimmune disease pathways of the autoimmune disease animal model comprises 1, 2, 3, 4, 5, 6, 7, 8, 9, 10, 11, 12, 13, 14, 15, 16, 17, 18, 19, 20, about 25, about 30, about 35, about 40, about 45, about 50, about 60, about 70, about 80, about 90, about 100, or more than about 100 different autoimmune disease pathways. In some embodiments, the one or more computer processors are individually or collectively programmed to further determine the up-regulation or down-regulation of the autoimmune disease pathway of the autoimmune disease animal model based on determining a difference between the quantitative measure of the animal genomic locus of the animal gene signature and a reference quantitative measure of the animal genomic locus. In some embodiments, the one or more computer processors are individually or collectively programmed to further obtain the reference quantitative measure of the animal genomic locus by, prior to (a), assaying an animal biological sample of the autoimmune disease animal model.

In another aspect, the present disclosure provides a non-transitory computer readable medium comprising machine-executable code that, upon execution by one or more computer processors, implements a method for identifying an autoimmune disease drug target, the method comprising: (a) obtaining gene expression data generated by assaying an animal biological sample of a treated animal model, wherein the treated animal model is obtained by treating an autoimmune disease animal model with a drug configured to inhibit a drug target of the autoimmune disease; (b) processing the gene expression data to obtain an animal gene signature, wherein the animal gene signature comprises quantitative measures of a first set of genomic loci associated with autoimmune disease pathways of the autoimmune disease animal model; (c) obtaining a set of human gene signatures, wherein the set of human gene signatures comprises quantitative measures of a second set of genomic loci associated with up-regulation or down-regulation of human autoimmune disease pathways in human patients having active autoimmune disease, and wherein the set of human gene signatures is generated by assaying human biological samples from one or more human patients having the autoimmune disease to obtain gene expression data; (d) processing the animal gene signature with the set of human gene signatures to identify (i) an animal genomic locus from among the first set of genomic loci, and (ii) a human genomic locus from among the second set of genomic loci that is associated with up-regulation or down-regulation of one or more human autoimmune disease pathways, wherein the animal genomic locus and the human genomic locus are orthologous and share similarity in expression patterns and function; and (e) identifying the drug target as the autoimmune disease drug target when the quantitative measure of the animal genomic locus of the animal gene signature is indicative of up-regulation or down-regulation of an autoimmune disease pathway of the autoimmune disease animal model.

In some embodiments, the autoimmune disease animal model is selected from: a mouse model, a rat model, a cat model, a dog model, a rabbit model, a guinea pig model, a hamster model, a pig model, a horse model, and a primate model. In some embodiments, the autoimmune disease animal model comprises a mouse model. In some embodiments, the autoimmune disease comprises lupus. In some embodiments, the lupus comprises systemic lupus erythematosus (SLE) or discoid lupus erythematosus (DLE). In some embodiments, the drug target is HDAC6. In some embodiments, the drug target is HDAC6 or a portion thereof. In some embodiments, the drug is an HDAC6 inhibitor. In some embodiments, the HDAC6 inhibitor is ACY-738. In some embodiments, the animal biological sample or the human biological samples comprise one or more of: a bodily fluid sample, a blood sample, a cell sample, and a tissue sample. In some embodiments, the one or more human autoimmune disease pathways are selected from the pathways listed in Table 61, Table 62, Table 63, and Table 64. In some embodiments, the human genomic locus that is associated with up-regulation or down-regulation of the one or more human autoimmune disease pathways is selected from the genes listed in Table 59, Table 60, Table 61, Table 62, Table 65, Table 66, and Table 67. In some embodiments, the autoimmune disease pathways of the autoimmune disease animal model are selected from the pathways listed in Table 61, Table 62, Table 63, and Table 64. In some embodiments, the animal genomic locus is selected from the genes listed in Table 59, Table 60, Table 61, Table 62, Table 65, Table 66, and Table 67. In some embodiments, (d) comprises identifying (i) a plurality of animal genomic loci from among the first set of genomic loci, and (ii) a plurality of human genomic loci from among the second set of genomic loci that is associated with up-regulation or down-regulation of a plurality of human autoimmune disease pathways, wherein plurality of animal genomic loci and the plurality of human genomic loci are pairwise orthologous and share similarities in expression patterns and function; and (e) comprises identifying the drug target as the autoimmune disease drug target when the quantitative measures of the plurality of animal genomic loci of the animal gene signature are indicative of up-regulation or down-regulation of a plurality of autoimmune disease pathways of the autoimmune disease animal model. In some embodiments, the plurality of human autoimmune disease pathways comprises between 2 and 5 different human autoimmune disease pathways. In some embodiments, the plurality of human autoimmune disease pathways comprises 1, 2, 3, 4, 5, 6, 7, 8, 9, 10, 11, 12, 13, 14, 15, 16, 17, 18, 19, 20, about 25, about 30, about 35, about 40, about 45, about 50, about 60, about 70, about 80, about 90, about 100, or more than about 100 different human autoimmune disease pathways. In some embodiments, the autoimmune disease pathways of the autoimmune disease animal model comprises 1, 2, 3, 4, 5, 6, 7, 8, 9, 10, 11, 12, 13, 14, 15, 16, 17, 18, 19, 20, about 25, about 30, about 35, about 40, about 45, about 50, about 60, about 70, about 80, about 90, about 100, or more than about 100 different autoimmune disease pathways. In some embodiments, the method further comprises determining the up-regulation or down-regulation of the autoimmune disease pathway of the autoimmune disease animal model based on determining a difference between the quantitative measure of the animal genomic locus of the animal gene signature and a reference quantitative measure of the animal genomic locus. In some embodiments, the method further comprises obtaining the reference quantitative measure of the animal genomic locus by, prior to (a), assaying an animal biological sample of the autoimmune disease animal model.

In another aspect, the present disclosure provides a method for evaluating a drug candidate for an autoimmune disease, the method comprising: (a) treating an autoimmune disease animal model with the drug candidate for the autoimmune disease, thereby producing a treated animal model; (b) assaying an animal biological sample of the treated animal model to obtain gene expression data of the treated animal model; (c) processing the gene expression data to obtain an animal gene signature, wherein the animal gene signature comprises quantitative measures of a first set of genomic loci associated with autoimmune disease pathways of the autoimmune disease animal model; (d) obtaining a set of human gene signatures, wherein the set of human gene signatures comprises quantitative measures of a second set of genomic loci associated with up-regulation or down-regulation of human autoimmune disease pathways in human patients having active autoimmune disease, and wherein the set of human gene signatures is generated by assaying human biological samples from one or more human patients having the autoimmune disease to obtain gene expression data; (e) processing the animal gene signature with the set of human gene signatures to identify (i) an animal genomic locus from among the first set of genomic loci, and (ii) a human genomic locus from among the second set of genomic loci that is associated with up-regulation or down-regulation of one or more human autoimmune disease pathways, wherein the animal genomic locus and the human genomic locus are orthologous and share similarity in expression patterns and function; and (f) evaluating the efficacy of the drug candidate for the autoimmune disease based at least in part on whether the quantitative measure of the animal genomic locus of the animal gene signature is indicative of up-regulation or down-regulation of an autoimmune disease pathway of the autoimmune disease animal model.

In another aspect, the present disclosure provides a computer-implemented method for evaluating a drug candidate for an autoimmune disease, the method comprising: (a) obtaining gene expression data generated by assaying an animal biological sample of a treated animal model, wherein the treated animal model is obtained by treating an autoimmune disease animal model with the drug candidate for the autoimmune disease; (b) processing the gene expression data to obtain an animal gene signature, wherein the animal gene signature comprises quantitative measures of a first set of genomic loci associated with autoimmune disease pathways of the autoimmune disease animal model; (c) obtaining a set of human gene signatures, wherein the set of human gene signatures comprises quantitative measures of a second set of genomic loci associated with up-regulation or down-regulation of human autoimmune disease pathways in human patients having active autoimmune disease, and wherein the set of human gene signatures is generated by assaying human biological samples from one or more human patients having the autoimmune disease to obtain gene expression data; (d) processing the animal gene signature with the set of human gene signatures to identify (i) an animal genomic locus from among the first set of genomic loci, and (ii) a human genomic locus from among the second set of genomic loci that is associated with up-regulation or down-regulation of one or more human autoimmune disease pathways, wherein the animal genomic locus and the human genomic locus are orthologous and share similarity in expression patterns and function; and (e) evaluating the efficacy of the drug candidate for the autoimmune disease based at least in part on whether the quantitative measure of the animal genomic locus of the animal gene signature is indicative of up-regulation or down-regulation of an autoimmune disease pathway of the autoimmune disease animal model.

In another aspect, the present disclosure provides a computer system for evaluating a drug candidate for an autoimmune disease, comprising: a database that is configured to store gene expression data generated by assaying an animal biological sample of a treated animal model, wherein the treated animal model is obtained by treating an autoimmune disease animal model with the drug candidate for the autoimmune disease; and one or more computer processors operatively coupled to the database, wherein the one or more computer processors are individually or collectively programmed to: (i) process the transcriptomic data to obtain an animal gene signature, wherein the animal gene signature comprises quantitative measures of a first set of genomic loci associated with autoimmune disease pathways of the autoimmune disease animal model; (ii) obtain a set of human gene signatures, wherein the set of human gene signatures comprises quantitative measures of a second set of genomic loci associated with up-regulation or down-regulation of human autoimmune disease pathways in human patients having active autoimmune disease, and wherein the set of human gene signatures is generated by assaying human biological samples from one or more human patients having the autoimmune disease to obtain gene expression data; (iii) process the animal gene signature with the set of human gene signatures to identify (1) an animal genomic locus from among the first set of genomic loci, and (2) a human genomic locus from among the second set of genomic loci that is associated with up-regulation or down-regulation of one or more human autoimmune disease pathways, wherein the animal genomic locus and the human genomic locus are orthologous and share similarity in expression patterns and function; and (iv) evaluate the efficacy of the drug candidate for the autoimmune disease based at least in part on whether the quantitative measure of the animal genomic locus of the animal gene signature is indicative of up-regulation or down-regulation of an autoimmune disease pathway of the autoimmune disease animal model.

In another aspect, the present disclosure provides a non-transitory computer readable medium comprising machine-executable code that, upon execution by one or more computer processors, implements a method for evaluating a drug candidate for an autoimmune disease, the method comprising: (a) treating an autoimmune disease animal model with the drug candidate for the autoimmune disease, thereby producing a treated animal model; (b) assaying an animal biological sample of the treated animal model to obtain gene expression data of the treated animal model; (c) processing the gene expression data to obtain an animal gene signature, wherein the animal gene signature comprises quantitative measures of a first set of genomic loci associated with autoimmune disease pathways of the autoimmune disease animal model; (d) obtaining a set of human gene signatures, wherein the set of human gene signatures comprises quantitative measures of a second set of genomic loci associated with up-regulation or down-regulation of human autoimmune disease pathways in human patients having active autoimmune disease, and wherein the set of human gene signatures is generated by assaying human biological samples from one or more human patients having the autoimmune disease to obtain gene expression data; (e) processing the animal gene signature with the set of human gene signatures to identify (i) an animal genomic locus from among the first set of genomic loci, and (ii) a human genomic locus from among the second set of genomic loci that is associated with up-regulation or down-regulation of one or more human autoimmune disease pathways, wherein the animal genomic locus and the human genomic locus are orthologous and share similarity in expression patterns and function; and (f) evaluating the efficacy of the drug candidate for the autoimmune disease based at least in part on whether the quantitative measure of the animal genomic locus of the animal gene signature is indicative of up-regulation or down-regulation of an autoimmune disease pathway of the autoimmune disease animal model.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The patent application file contains at least one drawing executed in color. Copies of this patent application publication with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows an example of a flow chart for a method of identifying one or more records, in accordance with disclosed embodiments.

FIG. 2A shows the z-scores determined by an example of differential expression analysis of disease state compared to status of the 100 most significant records within a first plurality of records, in accordance with disclosed embodiments.

FIG. 2B shows the z-scores determined by an example of differential expression analysis of active disease state compared to status of the 100 most significant records within a second plurality of records, in accordance with disclosed embodiments.

FIG. 2C shows the z-scores determined by an example of differential expression analysis of active disease state compared to status of the 100 most significant records within a third plurality of records, in accordance with disclosed embodiments.

FIG. 2D shows the z-scores determined by an example of differential expression analysis of active disease state compared to the combined records within the first, second, and third pluralities of records, in accordance with disclosed embodiments.

FIG. 2E shows the enrichment scores determined by an example of differential expression analysis of active disease state across a selected set of records compared to the first, second, and third pluralities of records, in accordance with disclosed embodiments.

FIG. 3 shows an example of a Venn diagram of the top 100 records within each of the first, second, and third pluralities of records, in accordance with disclosed embodiments.

FIG. 4A shows an example of Gene Set Enrichment Analysis (GSVA) enrichment scores and standard deviations for a first plurality of records, in accordance with disclosed embodiments.

FIG. 4B shows an example of GSVA enrichment scores and standard deviations for a second plurality of records, in accordance with disclosed embodiments.

FIG. 5 shows an example of Receiver Operating Characteristic (ROC) curves and the area under each curve for machine learning classifiers under different test conditions, in accordance with disclosed embodiments.

FIG. 6A shows an example of variable importance values of records as determined by mean decrease in Gini impurity, in accordance with disclosed embodiments.

FIG. 6B shows an example of variable importance values of de-duplicated records as determined by mean decrease in Gini impurity, in accordance with disclosed embodiments.

FIG. 6C shows an example of variable importance values of the top 25 individual genes determined by mean decrease in Gini impurity, in accordance with disclosed embodiments.

FIG. 7 shows a non-limiting schematic diagram of a digital processing device; in this case, a device with one or more CPUs, a memory, a communication interface, and a display;

FIG. 8 shows a non-limiting schematic diagram of a web/mobile application provision system; in this case, a system providing browser-based and/or native mobile user interfaces; and

FIG. 9 shows a non-limiting schematic diagram of a cloud-based web/mobile application provision system; in this case, a system comprising an elastically load balanced, auto-scaling web server and application server resources as well synchronously replicated databases.

FIG. 10A shows an example of heatmaps of −log 10(overlap p values) from RRHO, in accordance with disclosed embodiments. Strongest overlaps near the center of each plot indicate weak agreement among the most significantly upregulated and downregulated genes from each data set. Strong agreement between data sets may be indicated by a diagonal from the bottom-left corner to the top-right corner.

FIG. 10B shows an example of clustering all three studies on three consistent DE genes, in accordance with disclosed embodiments. DNAJC13, IRF4, and RPL22 were consistently differentially expressed in each study yet fail to fully separate active from inactive patients. Orange bars denote active patients; black bars denote inactive patients. Blue, yellow, and red bars denote patients from GSE39088, GSE45291, and GSE49454, respectively.

FIG. 11 shows GSVA results of a lupus Illuminate gene set, demonstrating the striking heterogeneity in SLE patient WB by showing patient specific enrichment of 27 cell and process specific modules of genes. In order to understand pathogenic mechanisms of SLE, a big data analysis approach may be used on purified cell populations implicated in SLE to help understand aberrant cellular-specific mechanisms.

FIG. 12 shows an example of cellular gene modules providing a basis for machine learning predictions of SLE activity, in accordance with disclosed embodiments. GSVA was performed on three SLE WB datasets using 25 WGCNA modules made from purified SLE cells with correlation or published relationship to SLEDAI. Orange: active patient; black: inactive patient. LDG: low-density granulocyte; PC: plasma cell.

FIGS. 13A and 13B show an example of individual WGCNA modules being ineffective at separating active and inactive SLE subjects, in accordance with disclosed embodiments. GSVA enrichment scores for CD4_Floralwhite (FIG. 13A) and CD4_0 rangered4 (FIG. 13B) in SLE WB are unable to fully separate active patients from inactive patients. Asterisks denote significant differences by Welch's t-test. Error bars indicate mean±standard deviation.

FIG. 14 shows an example of performance of machine learning classifiers across three independent data sets, in accordance with disclosed embodiments. Classifiers were trained on the data sets listed across the top and evaluated in the data sets listed across the bottom. Data sets are listed by their GEO accession numbers. Expression (black): gene expression data. WGCNA (blue): module enrichment scores.

FIG. 15 shows an example of area under the ROC curve of machine learning classifiers across three independent data sets, in accordance with disclosed embodiments. Classifiers were trained on the data sets listed across the top and tested in the other two data sets. Data sets are listed by their GEO accession numbers. Expression (black): gene expression data. WGCNA (blue): module enrichment scores.

FIGS. 16A-16C show an example of random forest classifier revealing variable importance of genes and modules, in accordance with disclosed embodiments. FIG. 16A shows variable importance of top 25 individual genes as determined by mean decrease in Gini impurity. FIG. 16B shows variable importance of cell modules. FIG. 16C shows that many modules shared genes, modules were de-duplicated to determine the effects on the random forest classifier. The relative importance of the full modules and de-duplicated modules was strongly correlated (Spearman's rho=0.69, p=1.94E-4). LDG: low-density granulocyte; PC: plasma cell.

FIG. 17 shows a heat map showing the variation of gene expression in normal controls. Differentially expressed (DE) transcripts pertaining to cell type and process signatures in 10 SLE whole blood and peripheral blood mononuclear cell microarray datasets were used to create modules of genes potentially enriched in SLE patients determined by Gene Set Variation Analysis (GSVA).

FIG. 18 shows PCA and heatmap clustering of AA, EA, and NAA SLE patients for 11 GSVA enrichment modules negative in healthy controls (HC). GSVA enrichment scores were uploaded to ClustVis, and PCA plots were generated.

FIG. 19 shows PCA and heatmap clustering of AA, EA, and NAA SLE Patients not taking steroids for 9 GSVA enrichment modules negative in healthy controls (HC). The cell cycle and Low Up modules were removed, GSVA enrichment scores for the 9 remaining modules were uploaded to ClustVis, and PCA plots and heatmaps were generated. Heatmaps were generated using correlation clustering distance for both rows and columns.

FIG. 20 shows PCA and heatmap clustering of a second, independent microarray dataset demonstrate that SLE patients divided into plasma cell or myeloid lupus. 73 AA and 71 EA patients from GSE45291 with SLEDAI in the range of 2-11 had GSVA scores calculated for 10 signatures. ClustVis was used to determine PC1 and PC2 for AA (top left) and EA (top right).

FIG. 21 shows heatmap clustering of SLE patients by enrichment of 10 immunologically related modules. SLE patients were grouped on the basis of having a negative PC1 loading score (plasma cell, left), a positive PC1 loading score (myeloid, middle), no enrichment of the 10 modules (No Sig, right). SLE patients within Plasma Cell or Myeloid that also expressed the opposite signature, as defined by either having a Mono GSVA enrichment score of at least 0.1, are identified by black boxes.

FIGS. 22A-22B show heatmap clustering of SLE patients by enrichment of 10 immunologically related modules. Four divisions were found for the 1,566 female SLE patients enrolled in the ILL clinical trials. Based on PC1 loadings for PCA of patients, PC and myeloid SLE patients were sorted by the opposite GSVA enrichment signature: monocyte cell surface for the PC signature (PCA PC1−) and Ig for the myeloid signature (PCA PC1+), and SLE patients with GSVA enrichment scores of at least 0.1 for the opposite signature were removed and reclassified as having both signatures (FIG. 22A). SLE patients of all ancestries were grouped based on the four classifications. ANOVA and Tukey's multiple comparisons test was performed between the four groupings (FIG. 22B).

FIGS. 23A-23D show the correlation between clinical measures of disease activity and WGCNA modules. Patients were divided into sub-groups based on their expression of positive eigengenes for each category. Significant differences between clinical traits were determined between group using PRISM v7 Tukey's multiple comparison test, and p values are shown between groups when less than or equal to 0.05.

FIG. 24 shows mean GSVA scores of patients in each cluster defined by GMM. Numbers at the top denote the number of patients in each cluster.

FIG. 25 shows gene expression of subjects in groups defined by GMVAE. GSVA analysis of the patients in these clusters showed that the patients without serological SLE activity (clusters 3 and 5) also did not show immunological activity by gene expression, whereas the other clusters did show immunological activity.

FIGS. 26A-26D show limma differential expression (DE) analysis of AA, EA, and NAA SLE patients to each other, including determining thousands of DE transcripts for each ancestry compared to the others for the ILL1 dataset.

FIG. 27A shows that in EA SLE patients, transcripts for monocytes and low-density granulocytes (LDGs) were enriched in the ILL1 and ILL2 datasets compared to AA SLE patients, whereas T cell and MHC class II transcripts were enriched in EA patients compared to NAA patients. NAA patients had increased myeloid signatures, including transcripts associated with monocytes, LDGs, and neutrophils compared to both AA and EA patients.

FIG. 27B shows that, similar to the results using the ILL1 and ILL2 datasets, EA SLE patients were enriched for transcripts associated with myeloid cells, and AA SLE patients were enriched for transcripts associated with plasma cells, B cells, and T cells.

FIG. 28A shows results of gene set variation analysis (GSVA) employed to compare enrichment of 34 modules of genes corresponding to lymphocytes, myeloid cells, cellular processes, as well as groups of all the T Cell Receptor (TCR) and immunoglobulin (Ig) genes found on the Affymetrix HTA2.0 array.

FIGS. 28B-28C show that the AA and NAA patient groups had significantly more SLE patients with platelet and erythrocyte enrichment than EA patients, and significantly fewer patients with decreased erythrocyte and platelet GSVA scores compared to EA patients.

FIG. 28D shows an orthogonal approach using weighted gene co-expression network analysis (WGCNA) to confirm the association of ancestry with cellular signatures. WGCNA of GSE88884 ILL1 and ILL2 was performed separately, and results demonstrated a significant (p<0.05) positive association by Pearson correlation of AA ancestry to plasma cell, T cell, and FOXP3 T cell modules, as well as a significant negative correlation to granulocyte and myeloid cell WGCNA modules.

FIG. 29 shows a comparison of patients on specific therapies to patients not receiving the therapies for the 34 cell type and process modules, in order to determine the effect of SOC drugs on patient gene expression signatures.

FIGS. 30A-30C show a comparison of LDG, monocyte, and T cell GSVA scores for patients with or without corticosteroids, demonstrating that the corticosteroids were the largest contributor to the differences between patient LDG, monocyte, and T cell scores, but that AA patients still had lower LDG and monocyte scores and NAA patients still had lower T cell scores in the absence of corticosteroids.

FIG. 30D shows that MTX and MMF significantly lowered plasma cell GSVA scores, but did not negate the increased plasma cells determined for AA patients versus EA and NAA patients.

FIG. 30E shows that compensating for AZA treatment also did not offset the increased B cells in AA SLE patients.

FIG. 30F shows that compensating for AZA treatment also did not offset the the difference in NK cells between EA and NAA SLE patients.

FIG. 31A shows a comparison of GSVA enrichment scores for the 34 modules for patients with each manifestation individually to all other manifestations, in order to determine the association between different SLE manifestations and gene expression profiles.

FIG. 31B shows a comparison of the change in gene expression profile for the anti-dsDNA, anti-RNP, or both, to the 64 patients in this subset without anti-RNP or anti-dsDNA autoantibodies showed significant increases in GSVA enrichment scores for IFN (anti-dsDNA, p=0.0023; anti-RNP, p=0.0323; both, p<0.0001), plasma cells (anti-dsDNA, p=0.01; anti-RNP and both, p<0.0001), Ig (anti-dsDNA, p=0.0039; anti-RNP and both, p<0.0001) and cell cycle (anti-dsDNA, p=0.0003; anti-RNP and both, p<0.0001).

FIG. 32A shows a comparison of patients positive for both Low C and anti-dsDNA with and without specific drugs or manifestations for cell specific GSVA scores, to determine whether autoantibodies and complement levels or drugs contributed more to the relationship with specific GSVA signatures.

FIG. 32B shows that 90% of patients with both Low C and anti-dsDNA were also receiving corticosteroids, and patients taking corticosteroids had significantly increased LDG GSVA scores, demonstrating that the increase in LDGs observed in patients with anti-dsDNA and Low C was related to concomitant corticosteroid usage, and not the presence of anti-dsDNA and Low C.

FIGS. 32C-32D show that the increase in IFN signature observed in EA and AA SLE patients on corticosteroids was related to the disproportionate numbers of patients with Low C and anti-dsDNA in the corticosteroid population, 39%, versus only 13% of the patients not taking corticosteroids who had both Low C and anti-dsDNA.

FIGS. 32E-32F show that in EA SLE patients, decreased NK cells were detected in those with anti-dsDNA or Low C. The effect was related to 23% of patients with Low C and anti-dsDNA also being on AZA (FIG. 32E) compared to only 15% of patients without low C or anti-dsDNA taking AZA (FIG. 32F) and thus not directly related to having anti-dsDNA and Low C.

FIGS. 32G-32H show that separation of vasculitis patients by anti-dsDNA and Low C demonstrated that the significant increase in plasma cells and IFN GSVA scores were likely related to the patients also having both anti-dsDNA and Low C, as there was a significant increase in GSVA enrichment scores for IFN and plasma cells in vasculitis patients with both anti-dsDNA and Low C (plasma cell mean difference=0.2873, p=0.0013, IFN mean difference=0.3889, p<0.0001).

FIG. 33A shows GSVA enrichment scores calculated for the 34 cell and process modules for 14 AA, 93 EA, and 17 NAA GSE88884 ILL1 and ILL2 male patients and male HC, to determine whether ancestral differences are also observed in male lupus subjects.

FIG. 33B shows that the combination of anti-dsDNA and Low C was associated with positive plasma cell signatures, as was detected for female SLE patients.

FIGS. 33C-33E show results of using EA SLE patients to determine differences between female patients and male patients with SLE. Because of the large number of female patients, the sets of female patients and male patients were able to be balanced for the percentage of patients on corticosteroids, AZA, and MTX/MMF. Further, the female patients were divided into two age groups, 25-49 years and over 50 years, because of the effects of estrogen on immune responses.

FIG. 34A shows gene expression analysis of adult, self-described AA and EA HC subjects carried out on two separate microarray datasets of normal subjects of different ancestries, in order to demonstrate that gene expression differences detected between SLE patients are related to heritable differences manifesting in expressed genes in hematopoietic cells of healthy subjects of different ancestries.

FIG. 34B shows that I-scope analysis of the transcripts increased in healthy AA patients demonstrated an increase in B cell, dendritic, erythrocyte, and platelet associated transcripts compared to EA HC subjects, and an increase in granulocyte, monocyte, and myeloid transcripts in healthy EA subjects compared to AA HC subjects.

FIG. 35 shows a CIRCOS visualization of the odds ratios for each variable significantly (p<0.05) contributing to each GSVA enrichment score. Ancestry significantly influenced 21 of the 34 cell type and process module scores.

FIG. 36 shows that gene expression is affected by ancestry, SLE autoantibodies, and standard-of-care (SOC) drugs. Average difference in GSVA enrichment scores are shown for healthy subjects. Average GSVA enrichment scores are shown for lupus (SLE) patients.

FIG. 37 contains plots showing that GSVA demonstrates metabolic dysregulation in individual SLE affected tissues. GSVA enrichment scores were calculated for (A) glycolysis, (B) pentose phosphate, (C) tricarboxylic acid cycle (TCA), (D) oxidative phosphorylation, (E) fatty acid beta oxidation, and (F) cholesterol biosynthesis modules in DLE, LA, LN Glom, and LN TI.

FIGS. 38A-38C contains plots showing that GSVA reveals potential pathways for therapeutic targeting in lupus affected tissues. Measures are shown for drug pathways significantly enriched in SLE affected tissue compared to control tissue as determined using the Welch's t-test for B cell activating factor (BAFF) (FIG. 38A), interleukin (IL-6) (FIG. 38B), and CD40 signaling in DLE, LA, and LN Glom (FIG. 38C). ** p<0.01, *** p<0.001.

FIG. 38D shows that genes commonly dysregulated in lupus tissues identified immune processes and cellular metabolism.

FIG. 38E shows that functional grouping and pathway analysis of DE genes expressed in lupus tissues revealed immune and metabolic abnormalities in common.

FIG. 38F shows that similar cellular and metabolic signatures were observed in lupus tissues.

FIG. 38G shows that increased immune/inflammatory cell signatures were observed in lupus tissues.

FIG. 38H shows that decreased tissue stromal cell signatures were observed in lupus tissues.

FIG. 38I shows that decreased metabolic signatures were observed in lupus tissues.

FIG. 38J contains plots showing the correlation between immune/inflammatory or tissue cell signature and metabolic signature in DLE and LN (LN GL and LN TI).

FIG. 38K-38L shows that Classification and Regression Trees (CART) analysis predicted the contributors to metabolic dysfunction.

FIG. 38M shows that Class 2 LN glomerulus demonstrated similar metabolic defects, indicating dysregulation is linked to stromal cells.

FIG. 38N contains plots showing the correlation between tissue or immune/inflammatory cell signature and metabolic signature for Class 2 LN glomerulus.

FIG. 38O-38P contain plots showing that metabolic changes were not correlated with T Cells in LN GL.

FIG. 39 contains plots showing results from mapping a total of 908 Immunochip SNPs to 252 eQTLs and coupling them to 760 E-Genes (207 in EAs, 30 in AAs, 523 shared), including (A) a Venn of E-Gene overlap and (B) a Cytoscape visualization of E-Gene PPI networks using MCODE clustering.

FIGS. 40A-40C show a non-limiting example of using interferon (IFN) subtype signatures to separate SLE patients from healthy controls (HC), using the systems and methods herein. FIG. 40A is a Venn diagram of the overlap of transcripts induced in human PBMC after 24-hour treatment with IFNA2, IFNB1, IFNW1, or IFNG. A 200-gene signature common to the three type I IFNs (IFN Core, 146+54) was determined. Gene symbols for the induced transcripts for each IFN are listed in Tables 19-29. The induced transcripts from IFN or cytokine treatment of PBMC were used as enrichment groups for GSVA analysis of SLE patient PBMC (FDA PBMC) (FIG. 40B), or SLE whole blood (GSE49454) (FIG. 40C). A heatmap visualization uses red (enriched signature) for GSVA values above zero and blue (decreased signature) for GSVA values below zero to show differences between SLE patients and controls. SLE patients were considered positive for a signature if their GSVA enrichment score was greater than the average healthy control (HC) GSVA enrichment score plus two standard deviations. Most SLE patients displayed prominent type I IFN signatures. In patients SLE.9495 and SLE.9491, enriched PBMC-TNF signatures compared to IFN signatures are displayed, and patient SLE.9544* had no PBMC-IFN signature and was grouped with controls (FIG. 40C).

FIGS. 41A-41D show a non-limiting example of using three interferon subtype signatures (IFNA2, IFNB1, and IFNW1) to separate SLE patients from healthy controls (HC), using the systems and methods herein. GSVA enrichment scores were calculated using the PBMC IFNA2, IFNB1, IFNW1, IFNG, IL12, or TNF induced transcripts, and a random signature (Random Gr1) (Table SD2), for discoid lupus erythematosus (DLE) and healthy control (HC) skin (FIG. 40A), SLE synovium and osteoarthritis synovium (FIG. 40B), lupus nephritis (LN) glomerulus (Glom) class III/IV and HC Glom (FIG. 40C), and LN tubulointerstitium (TI) class III/W and HC tubulointerstitium (TI) (FIG. 40D). Hedge's G effect size (Effect) measures are shown for cytokine signatures significantly enriched in SLE affected tissues compared to control tissues as determined by a p value<0.05 using the Welch's t-test. For LN tissues, recalculation of effect size values without the five IFN negative tissues roughly doubled the effect size values for the type I IFNs. In particular, the effect size values obtained were: IFNW1 (Glom g=5.5, TI g=3.3), IL12 (Glom g=4.9, TI g=1.9); IFNG (Glom g=5.5, TI g=2.2), IFNB1 (Glom g=6.0, TI g=3.0), IFNA2 (Glom g=6.6, TI g=3.1), but they were still lower than the effect size values calculated for the DLE and SLE synovium.

FIGS. 42A-42E show a non-limiting example of using whole blood (WB) interferon (IFN) signatures induced in IFNA2-treated hepatitis C (HepC) patients and IFNB1-treated multiple sclerosis (MS) patients to separate SLE patients from healthy controls (HC), using the systems and methods herein. FIG. 42A is a Venn diagram of the overlapping increased transcripts from MS-IFNB1, HepC-IFNA2, IFNA2, IFNB1, and IFNW1 signatures. FIGS. 42B-42E show GSVA using the increased transcripts of MS-IFNB1, HepC-IFNA2, and the transcripts from either signature restricted to only genes listed on the Interferome (Ifome; www.interferome.org) for DLE and HC skin (FIG. 42B), SLE synovium and OA (FIG. 42C), LN Glom Class III/W and HC Glom (FIG. 42D), and LN TI Class III/W and HC TI (FIG. 42E). Hedge's G effect size measures are shown for IFN signatures significantly enriched in SLE affected tissues compared to control tissues as determined by a p value<0.05 using the Welch's t-test. For LN tissues, removal of the five IFN negative SLE tissues doubled the effect size values for HepC-IFNA2 (Glom g=6.8, TI g=3.1) and MS-IFNB1 (Glom g=7.7, TI g=3.2).

FIG. 43 shows a non-limiting example of measuring a strong IFNB1 signature in cells and tissues from SLE patients, using the systems and methods herein. Z scores were calculated using the differential expression (DE) results from human PBMC treated with IFNA2, IFNB1, IFNW1, IFNG, IL12, TNF, MS patients treated with IFNB1 (MS-IFNB1), sepsis PBMC (control), and dermatomyositis skin (control) for SLE WB, PBMC, and affected tissues. Z scores>2 are considered significant. WB and PBMC datasets from active (SLEDAI≥6) and inactive (SLEDAI<6) SLE patients were divided and compared to the same controls separately before Z scores were calculated.

FIG. 44 shows a non-limiting example that IGS is readily detected in active and inactive SLE patients, using the systems and methods herein. Seven SLE datasets were divided into active SLE patients with SLEDAI≥6 (1722 patients total), or inactive SLE patients with SLEDAI<6 (315 patients total). GSVA enrichment scores were calculated for each patient using the IFN Core signature (such as IFNA2, IFNB1, IFNW1, MS-IFNB1, and HepC-IFNA2 signatures). IFN core positive patients had GSVA enrichment scores greater than 2 standard deviations from the average of the CTL GSVA enrichment scores.

FIGS. 45A-45F show a non-limiting example that SLE patients may lose or gain the IGS over time, using the systems and methods herein. An F test differential expression (DE) analysis of SLE patients on standard of care (SOC) treatment at zero weeks, 16 weeks, and 52 weeks from SLE time course datasets GSE88885 and GSE88886 was carried out, and GSVA enrichment scores were calculated using the IFN core signature. The dotted line represents the average IFN core GSVA score for the controls, but only patients are shown in the graphs. Changes in the IGS score of greater than 0.2 standard deviations were considered significant. For the GSE88885 SLE dataset, 54 SLE patients had minimal changes in their IGS (FIG. 45A), 18 SLE patients changed from negative to positive score (FIG. 45B), and 14 SLE patients changed from positive to negative enrichment score (FIG. 45C). For the GSE88886 SLE dataset, 23 SLE patients had minimal changes in their IFN core GSVA enrichment score (FIG. 45D), five SLE patients changed from negative to positive (FIG. 45E), and five SLE patients changed from positive to negative IGS enrichment score (FIG. 45F).

FIGS. 46A-46F show a non-limiting example that the IGS and SLEDAI do not change synchronously, using the systems and methods herein. Ten SLE LN patients with SLEDAI>6 (GSE72747) and healthy controls (HC) (n=46) from GSE39088 had F test differential expression (DE) analysis using time zero, 12-week, and 24-week WB samples (Treatment with high-dose immunosuppressive was begun after time zero and continued for 12 weeks; at 12 weeks, all patients were switched to lower dose/maintenance therapy). Graphs show the change in SLEDAI versus the change in the IFN core signature GSVA enrichment score (FIGS. 46A-46B). GSVA enrichment signatures corresponding to B cells, T cells, plasma cells, and monocytes were determined at each time-point, and most patients had standard deviations>0.2 between their zero and 12-week time-points (FIGS. 46C-46F). One-way ANOVA p values were <0.05 for comparison of mean GSVA enrichment scores for B cells, T cells, and monocytes between time zero and 12 weeks. Tukey's multiple comparison test between time zero and 12 weeks showed significant differences in mean GSVA enrichment scores for B cells (p=0.02), T cells (p=0.03), and monocytes (p=0.05), but not plasma cells.

FIGS. 47A-47C show a non-limiting example of performing linear regression analysis to demonstrate that the IFN signature is most closely related to monocyte cell surface transcripts, using the systems and methods herein. Linear regression analysis using SLEDAI values from the patients of 5 SLE WB and 2 SLE PBMC datasets and the patient GSVA enrichment scores for cell type-specific signatures. FIG. 47A: Cell types or signatures with significant non-zero slopes (p<0.05) related to SLEDAI by linear regression analysis in at least half of the datasets which had determinable GSVA scores were used to determine overall significance of the regression lines and the r² predictive values for all 7 SLE datasets with available SLEDAI information. FIG. 47B shows a representative plot using the HepC-IFNA2 signature for the linear regression analysis between the IFN signature with overlapping transcripts to the cell type or process signatures removed and the cell type or process GSVA enrichment score for the patients from 10 SLE WB and PBMC datasets. Cell types or signatures significantly (p<0.05) related to HepC-IFNA2 score in at least half of the datasets which had determinable GSVA scores were used to determine overall regression lines for all 10 datasets. r² predictive values are listed after the GSVA enrichment category. Relationships and linear regression analysis can be performed likewise for the other IFN signatures. For time-course dataset GSE72747, linear regression analysis was done for the change in the core IFN GSVA score versus the change in monocyte cell surface score between 0 and 12 weeks and between 12 and 24 weeks (FIG. 47C).

FIGS. 48A-48G show a non-limiting example that monocytes from inactive SLE patients have an interferon signature and elevated STAT1 transcripts, using the systems and methods herein. WGCNA was performed on datasets GSE38351 CD14+ monocytes (6 active (SLEDAI≥6), 6 inactive (SLEDAI<6), and 12 control), GSE10325 CD4+ T cells (8 active, 4 inactive, and 9 control), and GSE10325 CD19+ B cells (10 active, 4 inactive, and 9 control), and individual patient eigengene values are shown for the IFN module from each dataset (FIGS. 48A-48C). The modules were correlated to presence of SLE disease (versus control) or the SLEDAI, and Pearson r values are shown for significant correlations for each WGCNA dataset (p<0.05). “NS” means not significant. SLEDAI values for each patient are listed at the end of the patient number with controls and patients with inactive disease (SLEDAI<6) noted by underlined text. GSVA enrichment scores were calculated using the IFN core signature for SLE and control samples of CD4+ T cells (FIG. 48D), CD19+ B cells (FIG. 48E), and CD14+ monocytes (FIG. 48F). Tukey's multiple comparisons test was used to determine significant differences between mean GSVA scores between controls, inactive and active patients. “*” indicates a p-value of <0.05 between active SLE and control or between inactive SLE and control; “**” indicates a p-value of <0.05 between active SLE and inactive SLE or between active SLE and control. Datasets of SLE WB, PBMC, purified CD14+ monocytes, T cells, and B cells were divided into active (SLEDAI≥6) and inactive (SLEDAI<6) for differential expression (DE) analysis to controls (FIG. 48G). The log fold change (LFC) for STAT1 is reported for each active and inactive dataset.

FIG. 49 shows a non-limiting example of transcripts from the in vitro treatment of PBMC with IFNA2, IFNB1, IFNW1, and IFNG (as described by, for example, Waddell, S. J. et al. Interferon-induced transcriptional programs in human peripheral blood cells. PLoS One 5(3): e9753(2010), which is hereby incorporated by reference in its entirety). Transcripts increased by a minimum fold change of 2 at a false discovery rate of 0.05 compared to mock treated PBMC. Unique transcripts for IFNA2, IFNB1, IFNW1, and IFNG were determined by comparison of the four signatures. The heatmap scale represents fold change.

FIGS. 50A-50E show a non-limiting example that Chiche-Chaussable modules do not reflect a specific sub-type of IFN. Shown are the overlap of the three Chiche-Chaussabel interferon modules (IFN-M) with the Waddell transcripts induced by IFNA2 (FIG. 50A), IFNB1 (FIG. 50B), IFNW1 (FIG. 50C), and IFNG (FIG. 50D). Each IFN-M overlapped the IFNA2, IFNB, and IFNW1 signatures with the same genes, except IFI44L from M1.2 was only in IFNA2 and DRAP1, NBN and IRF9 from M5.12 were only found in the IFNB1-induced transcripts. Overlapping genes were found within the core IFN genes, not the unique IFN signatures (FIG. 50E).

FIG. 51 shows a non-limiting example that GSVA enrichment using random genes does not separate SLE patients from controls. Shown are heatmap visualization of the GSVA enrichment scores for the Waddell IFNB1 increased transcripts (IFNB1) and two groups of random, not co-expressed transcripts derived from random sorting of dataset GSE49454 differential expression (DE) transcripts. Enrichment scores were calculated using these groups for all patients and controls in dataset GSE49454 (n=46).

FIGS. 52A-52D show a non-limiting example that a DMS-IFNB1 signature in multiple sclerosis (MS) patient whole blood (WB) confirms a strong IFNB1 signature. Shown are linear regression analysis using the MS-IFNB1 signature of increased and decreased transcripts with SLE Active (SLEDAI≥6) whole blood (WB) (FIG. 52A), SLE active PBMC (FIG. 52B), DLE (FIG. 52C), and sepsis (FIG. 52D).

FIGS. 53A-53B show a non-limiting example that an MS-IFNB1 signature separates SLE cells and tissues. Shown are GSVA results using the MS-IFNB1 signature. Increased (IFNB UP) and decreased (IFNB DOWN) transcripts (DE to untreated multiple sclerosis patients) separated SLE patients from healthy controls (HC) in WB GSE49454 active (SLEDAI≥6) SLE patients (n=23) (FIG. 53A), and DLE GSE72535 (n=9) (FIG. 53B).

FIGS. 54A-54D show a non-limiting example that the alternative IFNB1 downstream signaling pathway does not predominate in SLE tissues. Murine IFN alpha/beta receptor 2 deficient mice were injected with IFNB1 into the peritoneum, and peritoneal exudate cells (PEC) were isolated for microarray expression analysis to control PEC. Increased transcripts induced by IFNB1 signaling through the IFN alpha/beta receptor 1 only were used as a GSVA enrichment group to determine if the alternative pathway of IFNB1 signaling was contributing to gene regulation in DLE (FIG. 54A), SLE synovium (FIG. 54B), LN Glom class III/IV (FIG. 54C), and LN TI class III/IV (FIG. 54D). Hedge's G effect size measures (Effect) are shown for tissues with significant (p<0.05) differences between the mean GSVA enrichment scores between SLE affected and control tissues by Welch's t-test. “N/A” denotes not applicable due to insignificant Welch's t-test value.

FIGS. 55A-55E show a non-limiting example that the IGS and SLEDAI do not change synchronously. Ten SLE lupus nephritis patients with SLEDAI>6 (GSE72747) had F test differential expression (DE) analysis using time zero, 12-week and 24-week time points. Treatment with high-dose immunosuppressive was begun after time zero and continued for 12 weeks; at 12 weeks, all patients were switched to lower dose/maintenance therapy; healthy controls from the GSE39088 dataset were included in the analysis. Graphs show the change in SLEDAI versus the change in the GSVA enrichment scores for 0 to 12 weeks (top), and for 12 to 24 weeks (bottom) for MS-IFNB1 (FIG. 55A), HepC-IFNA2 (FIG. 55B), IFNA2 (FIG. 55C), IFNB1 (FIG. 55D), and IFNW1 (FIG. 55E).

FIGS. 56A-56E show a non-limiting example that IFN subtypes are most related to monocyte cell surface transcripts by linear regression analysis. Shown are linear regression analysis results between the cell type-specific, nonoverlapping IFN signatures, and the GSVA enrichment cell type score (y-axis) for the patients from 10 SLE WB and PBMC datasets. Cell types or signatures significantly (p<0.05) related to the nonoverlapping IFN score for MS-IFNB1 (FIG. 56A), type I IFN core (FIG. 56B), IFNA2 (FIG. 56C), IFNB1 (FIG. 56D), and IFNW1 (FIG. 56E) in at least half of the datasets which had determinable GSVA scores were used to determine overall regression lines for all 10 datasets. The r² values are listed after the GSVA enrichment category. “PC” indicates plasma cell, “UPR” indicates unfolded protein response, and “LDG” indicates low density granulocyte.

FIGS. 57A-57B show a non-limiting example of using LDG-specific genes to compare low-density granulocyte (LDG) differentially expressed genes (DEGs) relative to SLE neutrophils and healthy control (HC) neutrophils, using the systems and methods herein. Shown is a comparison of LDG upregulated genes versus SLE neutrophils or HC neutrophils by limma analysis. Genes were considered upregulated or downregulated if they had an FDR<0.05. FIG. 57A shows a comparison of LDG genes upregulated versus SLE neutrophils or HC neutrophils. FIG. 57B shows a comparison of LDG genes downregulated versus SLE neutrophils or HC neutrophils.

FIGS. 58A-58B show a non-limiting example of using weighted gene coexpression network analysis (WGCNA) module eigengene (ME) values to separate LDGs from both SLE neutrophils and HC neutrophils, using the systems and methods herein. Samples from GSE26975 were used in two separate WGCNA analyses to examine LDGs and HC or LDGs and SLE neutrophils. Module colors are assigned by the WGCNA pipeline based on module size. Eigengene values separate LDGs from HC neutrophils (n=9 HC, 10 LDG) (FIG. 2A) and SLE neutrophils (n=10 SLE, 10 LDG) (FIG. 2B) by Welch's t test (*p<0.05).

FIGS. 59A-59D show a non-limiting example of grouping LDG WGCNA modules by eigengene values and constituent genes, using the systems and methods herein. LDG eigengene values for pink and black modules (FIG. 59A) or grey60 and green-yellow modules (FIG. 59B) demonstrate that the four WGCNA modules can be broken into two groups based on the behavior of their eigengenes from patient to patient. Pearson r and p values are shown. WGCNA modules with highly correlated eigengenes have many genes in common. LDG module A was formed from the genes shared between the pink and black modules (FIG. 59C). LDG module B was formed from the genes shared between the grey60 and green-yellow modules (FIG. 59D).

FIGS. 60A-60C show a non-limiting example of performing STRING/MCODE functional analysis of LDG module B to elucidate two major clusters characterized by cell cycle and neutrophil degranulation, using the systems and methods herein. MCODE clustering was used to identify the most strongly connected members of module B's STRING protein-protein interaction network. The top cluster (FIG. 60A) has many genes associated with the cell cycle by GO (diamonds). The bottom cluster (FIG. 60B) is almost entirely composed of genes associated with neutrophil degranulation (squares). Cell cycle and neutrophil degranulation genes not connected to an MCODE cluster are shown on the right. The presence of neutrophil-associated genes in module B led to its selection as the module used to query blood and tissue gene expression data. A gene ontology designation is shown in FIG. 60C, where genes associated with cell cycle are denoted by diamonds, genes associated with neutrophil degranulation are denoted by squares, and genes having other ontologies are denoted by circles.

FIG. 61 shows a non-limiting example of computational and functional analyses to study the relationships between module enrichment and disease manifestations in SLE whole blood, using the systems and methods herein. Shown is a flow chart illustrating the process of generating, filtering, and analyzing WGCNA gene modules. Modules are evaluated by functional analysis and tests of co-expression in blood and tissue data sets. GSVA enrichment scores are used to study the relationships between module enrichment and disease manifestations in SLE whole blood.

FIGS. 62A-62F show a non-limiting example of determining that LDG Modules are associated with platelet counts or neutrophil counts in GSE49454 WB, using the systems and methods herein. Shown are LDG Module A enrichment score versus platelet counts (FIG. 62A), neutrophil counts (FIG. 62B), and neutrophil counts (FIG. 62C) excluding patients with counts less than 1,500/mm³ or greater than 8,000/mm³. FIGS. 62D-62F show an analysis of LDG Module B enrichment scores.

FIG. 63 shows a non-limiting example of a method for identifying a lupus condition of a subject using PID profiling, in accordance with disclosed embodiments.

FIG. 64 shows a non-limiting example of cross-checking primary immunodeficiency (PID) genes in 928 hematopoietic immune cells, in accordance with disclosed embodiments. The expression of the genes must be specific to hematopoietic cells, because if not restricted, then these genes could be targeted in non-immune specific cells and have detrimental effects.

FIG. 65A shows a non-limiting example of a database at large, comprising 432 genes, in accordance with disclosed embodiments. Via deliberation of various primary literature, the database was compiled with 432 PID-associated genes. Each PID gene includes characteristic information that can be used to identify and describe the gene.

FIGS. 65B-65C show a non-limiting example of a table of the database shown in FIG. 65A, in accordance with disclosed embodiments.

FIG. 66A shows a non-limiting example of results showing that some PID-associated genes are specific to immune hematopoietic stem cells, in accordance with disclosed embodiments. Of the 450 PID-associated genes, 125 genes were determined to be specific to immune hematopoietic cells. Of the 25 immune cell categories specific to hematopoietic cells and various cell types, the 125 genes are concentrated in monocyte, myeloid, B cell, T cell, and B and T cell categories.

FIG. 66B shows a non-limiting example of results showing the cell count per category of various cell types.

FIGS. 67A-67B show a non-limiting example of protein-protein interaction-based clustering of 450 PID-associated genes, in accordance with disclosed embodiments. Protein-protein interaction networks and clusters were generated via Cytoscape using the STRING and MCODE plugins. FIG. 67A shows that of the 450 genes, 430 genes were grouped into 16 clusters, and the BIG-C™ category most representative of the gene list was used to biologically characterize the clusters. The clusters with the most genes include clusters 1, 2, 3, 4, and 5. The BIG-C™ categories represented by these large clusters include immune cell surface, intracellular signaling, pattern recognition receptors, DNA repair, pro-proliferation, secreted immune, and extracellular matrix. The node sizes correlate to the number of genes in each cluster, and the degree of node shading indicates the number of intracluster connections (see gradient at bottom of figure). The edge weight thickness represents the number of intercluster connections. FIG. 67B shows that the 450 genes were grouped into 16 clusters. Data from GSE88884, which includes transcriptomic data of 1,620 patients, was used to determine the differential expression of the genes.

FIG. 68 shows a non-limiting example of endotypes of SLE patients defined by functional groupings of PID-associated genes, in accordance with disclosed embodiments. Differentially expressed (DE) genes from the GSE88884 SLE WB dataset (1,620 patients) were assessed by GSVA for the 17 MCODE clusters, as shown in FIGS. 67A-67B (and on the x-axis of the heatmap). There is a clear distinction between enrichment of the clusters among the patients, thereby demonstrating that these groups of immune-specific genes can be used to differentitate SLE patients based on clinical presentation of disease.

FIG. 69 shows a non-limiting example of performing GSVA to identify the functional role of PID-associated genes expressed in SLE WB microarray datasets, in accordance with disclosed embodiments. DE genes from 14 SLE WB datasets shown on the x-axis were overlapped with the 432 PID-associated genes to assess common genes. SLE WB DE genes that are also PID-associated genes were analyzed by GSVA for function by enrichment with BIG-C functional categories as shown on the y-axis. Welch's t test was used to identify significant BIG-C categories including interferon stimulated genes, MHC class-1 antigen presentation, secreted-immune, secreted extracellular matrix, pattern recognition receptors, proteasome activity, and pro-apoptosis.

FIG. 70 shows a non-limiting example of results demonstrating that PID-associated genes differentially expressed in a large whole blood dataset comprised of distinct patient groups, in accordance with disclosed embodiments.

FIG. 71 shows a non-limiting example of a workflow to assess a condition of a subject using one or more data analysis tools and/or algorithms, in accordance with disclosed embodiments.

FIG. 72 shows a non-limiting example of using BIG-C® to generate a differential expression heatmap, in accordance with disclosed embodiments.

FIG. 73 shows a non-limiting example of using BIG-C® to generate a gene coexpression plot, in accordance with disclosed embodiments.

FIG. 74 shows a non-limiting example of using BIG-C® to cross-examine enriched categories with GO and KEGG terms to derive key insights for further analysis, as shown by the enriched categories identified (left) and cross-referenced to GO terms, in accordance with disclosed embodiments.

FIG. 75 shows a non-limiting example of an I-Scope™ signature analysis for a given sample, in accordance with disclosed embodiments.

FIG. 76 shows a non-limiting example of an I-Scope™ signature analysis for a given sample across multiple samples and disease states, in accordance with disclosed embodiments.

FIG. 77 shows a non-limiting example of results obtained using T-Scope™ in combination with I-Scope™ for identification of cells post-DE-analysis, in accordance with disclosed embodiments.

FIG. 78 shows a non-limiting example of MS-Scoring™ 1 of IL-12 and IL-23 related pathways for targeting using ustekinumab for SLE (systemic lupus erythematosus) drug repositioning, in accordance with disclosed embodiments.

FIG. 79 shows a non-limiting example of results from GSVA Analysis on SLE (systemic lupus erythematosus) signaling pathways, in accordance with disclosed embodiments.

FIG. 80 shows a non-limiting example of the CoLT Scoring® of SOC Therapies in Lupus (Belimumab, HCQ, and Rituximab), in accordance with disclosed embodiments.

FIG. 81 shows a non-limiting example of the Target-Scoring categories and point values, in accordance with disclosed embodiments.

FIG. 82 shows results of LN differential gene expression. Microarray data from 30 LN patients and 14 healthy controls were processed by LIMMA to identify DE genes in microdissected glomeruli and TI from WHO classes 2a, 2b, 3, and 4.

FIGS. 83A-83B show generation of WGCNA gene modules from LN glomerular and tubulointerstitium (TI) differential expression (DE) data and correlation to clinical covariates.

FIGS. 84A-84B show GSVA enrichment and sorting of LN patients against WGCNA module membership.

FIG. 85 shows enrichment of functional categories in LN signatures via BIG-C®. Modules were characterized for patterns of member gene function via comparison to the BIG-C® database.

FIG. 86 shows enrichment of immune and tissue cell populations in LN signatures via I-Scope™ and T-Scope™.

FIG. 87 shows expression of PC and GC indicator genes in LN. To more closely and specifically interrogate LN samples for the presence and role of PCs, DE genes from LN glomeruli and TI across WHO classes were filtered against signatures for core plasma cell function, T follicular helper cells, and germinal center B cells.

FIGS. 88A-88E show patterns of upstream regulator activation in LN. IPA® UR analysis of DE genes from glomerular and TI samples across WHO classes produces five blocks of interest (FIGS. 88A-88E, respectively) for identifying shared and unique immune, inflammatory, and cytokine/chemokine pathways between tissues and levels of LN severity (p<0.01).

FIG. 89 shows LINCS analysis identifies priority targets and drugs in LN glomerular and TI via upstream regulators. DE genes were analyzed with the LINCS platform, which returns connectivity scores for genes and compounds based on similarity of input signatures to a database of experimental knockdown, overexpression, and drug treatment models.

FIGS. 90A-90C show an example of performing WGCNA to identify modules with significant correlations to clinical variables. Performing WGCNA identified 41 modules for GSE72535, 23 modules for GSE81071, and 30 modules for GSE52471.

FIGS. 91A-91G show an example of WGCNA modules interrogated using BIG-C® functional characterizations as well as I-Scope™ and T-Scope™ for specific cellular subsets. DLE-associated modules identified in WGCNA are characterized by BIG-C® (FIGS. 91A-91C) and I-Scope™ and T-Scope™ (FIGS. 91D-91F). Odds ratios above 1 are shown, and Fisher's exact tests with p-values below 0.05 are indicated with an asterisk (FIG. 91G).

FIG. 92 shows an example of expression of tissue-specific signatures in WGCNA modules interrogated by GSVA. Gene Set Variation Analysis (GSVA) was performed to find enrichment of tissue specific gene signatures in each module.

FIG. 93 shows an example of expression of PC and GC indicator genes in DLE. To more closely and specifically interrogate DLE samples for the presence and role of PCs, DE genes from each dataset were filtered against signatures for core plasma cell function, T follicular helper cells, and germinal center B cells.

FIGS. 94A-94B show an example of WGCNA modules statistically preserved between three analyses. Module preservation was performed for each pairwise combination of datasets. The preservation Zsummary statistic was used to determine significant preservation.

FIGS. 95A-95B show an example of IPA canonical pathway and upstream regulator (UR) analysis. IPA® canonical pathway and upstream regulator analysis was performed.

FIG. 96 shows a non-limiting example of a workflow to assess a condition of a subject using one or more data analysis tools and/or algorithms, in accordance with disclosed embodiments.

FIG. 97 shows the process of unpacking an SLE-associated SNP, in accordance with disclosed embodiments.

FIGS. 98A-98C show an example of mapping SNP associations to eQTLs and E-Genes, in accordance with disclosed embodiments. FIG. 98A shows a distribution of genomic functional categories for EA and AA SNP sets. “NT-R” is defined as Non-Traditional Regulatory: intronic or intergenic SNPs exhibiting strong regulatory potential, indicated by DNAse hypersensitivity, location within protein binding sites and evidence of epigenetic modification. “Other” non-coding regions include introns, intergenic regions, 5 kb upstream of transcription start sites and 5 kb downstream of transcription termination sites. FIG. 98B shows a summary of eQTL analysis. SLE-associated SNPs identify multiple eQTLs linked to E-Genes in the GTEx database. eQTLs and their associated E-Genes were divided into European ancestry (EA) and African ancestry (AA) groups depending on the ancestral origin of the original SLE-associated SNP. Shared E-Genes are derived from SNPs common to both EA and AA ancestries.

FIG. 98C shows the number of EA and AA SNPs mapping to single E-Genes, multiple E-Genes or shared E-Genes.

FIGS. 99A-99D show an example of E-Gene functional and pathway analysis, in accordance with disclosed embodiments. PANTHER (v. 13.1) was used to classify EA and AA E-Genes according to gene ontology (GO) biological processes and pathways. The number of EA (FIG. 99A) and AA (FIG. 99B) E-Genes assigned to GO biological processes is displayed in each bar graph; GO identifiers are reported to the right of each graph. For pathway analysis, EA (FIG. 99C) and AA (FIG. 99D) E-Gene sequences were assigned to GO pathways. EA E-genes are defined by 78 pathways; several pathways of interest containing 4 or more E-Genes are labeled. AA E-Genes are defined by 15 pathways as shown in the pie chart.

FIGS. 100A-100C show an example of generation of protein-protein interaction (PPI) networks, in accordance with disclosed embodiments. PPI networks and clusters generated were generated via CytoScape using the STRING and MCODE plugins. Networks were constructed of all EA, AA, and shared (EA+AA) E-Genes. MCODE clusters were determined by the strength of protein-protein interactions, calculated by pooling information from publicly available literature. FIG. 100A shows the cluster metastructure of each network and corresponding BIG-C™ categories, while FIGS. 100B-100C show the specific genes that make up each cluster. FIG. 100D shows EE, AA, and shared (EE+AA) E-Genes that were unclustered.

FIGS. 101A-101D show an example of a comparison of E-Genes predicted from SLE-associated SNPs with SLE differential expression datasets, in accordance with disclosed embodiments. Predicted E-Genes were matched with SLE differential expression (DE) data and organized by ancestry. FIG. 101A shows the fold-change variation of EA-only E-Genes. Due to the large number of DE EA E-Genes, a selection of the most highly upregulated and downregulated genes are presented. FIG. 101B shows AA-only DE E-Genes, and FIG. 101C shows DE E-Genes common to both the AA and EA gene sets. Color for all three heatmaps represents log fold change, as indicated by the legend underneath the central heatmap (FIG. 101D). Red asterisks indicate active SLEDAI datasets.

FIGS. 102-103 show an example of a comparison of E-Genes predicted from SLE-associated SNPs with SLE differential expression datasets, in accordance with disclosed embodiments. Compounds targeting EA, AA, shared tissue E-Genes and associated pathways are shown. Differentially expressed E-Genes from synovium, skin and kidney tissue datasets were first compared to immune-specific gene lists. Overlapping genes were used as input for IPA upstream regulator analysis. PPI networks and clusters were generated via CytoScape using the STRING and MCODE plugins. MCODE clusters were determined by the strength of protein-protein interactions, calculated by pooling information from publicly available literature. Select drugs acting on targets are shown. Where available, CoLT scores (−16 to +11) are depicted in superscript.

FIG. 104 shows a non-limiting example of a workflow to identify autoimmune disease drug targets, in accordance with disclosed embodiments.

FIGS. 105A-105E show a non-limiting example of results showing that inhibition of histone deacetylase HDAC6 reduced Ig and C deposition in NZB/W lupus nephritis. FIGS. 105A-105B show a representative Hematoxylin and Eosin (H&E) staining image of kidney glomerular region along with pathology score which reflects the severity of membranoproliferative changes and distribution. FIG. 105C shows a representative immunohistological staining of kidney section for IgG and C3. FIGS. 105D-105E show a graphic analysis of mean fluorescent intensity (MFI) of IgG and C3. Data are shown as mean±standard error of the mean (s.e.m) n=4 mice for each group; T-test; *P<0.05, **P<0.01, ****P<0.0001.

FIG. 106 shows a non-limiting example of results showing that HDAC6i treatment of NZB/NZW F1 mice induced global gene expression changes in whole splenocytes. Hierarchical clustering of 3911 transcripts (1922up, 1989 down) that differed significantly (FDR<0.1) between control (C1, C3, C4, and C5) and treated mice (T1, T2, T3, and T5).

FIGS. 107A-107D show a non-limiting example of results showing that HDAC6i treatment results in significantly decreased GC activity and PC formation. FIG. 107A shows results of I-Scope hematopoietic cell enrichment demonstrating that HDAC6 inhibition decreased PC, B cells, and inflammatory myeloid cells. The numbers of transcripts corresponding to each cell type increased or decreased after HDAC6 inhibitor treatment are shown. Gene symbols for transcripts for PC, B cells, and inflammatory myeloid cells are shown in Table 54 (increased transcripts) and Table 55 (decreased transcripts). FIG. 107B shows results of GSVA analysis performed to determine the enrichment of PC, Tfh cells, and GC in each HDAC6 inhibitor-treated and control NZB/NZW mouse (Methods lists genes used for GSVA enrichment modules). FIG. 107C shows a representative splenic section stained with anti-CD138, anti-IgM, and PNA. FIG. 107D shows a representative splenic section stained for T cells, follicular B cells, and GC with anti-CD3, anti-IgD, and PNA.

FIG. 108 shows a non-limiting example of results showing that HDAC6 inhibition repressed B cell signaling pathways in NZB/NZW mice. The IPA Canonical Signaling Pathway “B Cell Receptor Signaling” had a Z score of −3.1. Transcripts differentially expressed between HDAC6 inhibitor-treated and untreated NZB/NZW mice were overlaid on genes in the IPA pathway. Decreased transcripts are shown in green, while increased transcripts are shown in pink.

FIGS. 109A-109D show a non-limiting example of results showing that inhibition of HDAC6 altered transcripts associated with cellular metabolism. FIG. 109A shows results of an ingenuity pathway analysis (IPA) performed on the differentially expressed transcripts between HDAC6 inhibitor-treated and untreated NZB/NZW mice. The most significant signaling pathways increased or decreased by Z score analysis with an overlap p value≤0.05 are shown. The full list of significant increased and decreased pathways and the genes used to determine significance are in Table 56 (increased) and Table 57 (decreased). FIG. 109B shows results of a GO biological pathway enrichment analysis of the top most increased and decreased pathways by lowest overlap p value significance. A full list of GO biological pathways enriched (p<0.01) are in Table 5 (increased) and Table 59 (decreased). FIGS. 109C-109D show results of a BIG-C pathway enrichment performed using increased (FIG. 109C) or decreased (FIG. 109D) transcripts from the DE analysis of HDAC6 inhibitor-treated NZB/NZW mice compared to NZB/NZW mice. The −log (p value) is shown for the enriched categories. Gene symbols corresponding to each category are listed in Table 60 (increased) and Table 61 (decreased).

FIGS. 110A-110C show a non-limiting example of results showing that HDAC6 inhibition decreased citrate synthase activity and cytochrome c oxidase activity in NZB/W mice. Four weeks of treatment of NZB/W mice with the HDAC6 inhibitor ACY-738 lead to a significant decrease in the rate limiting enzyme of the TCA cycle (p=0.043) (FIG. 110A), and a decrease in cytochrome C oxidase activity (P=0.053) (FIG. 110B), while having minimal effect on beta hydroxyacyl coa dehydrogenase in splenocytes (n=5) (FIG. 110C).

FIGS. 111A-111B show a non-limiting example of results showing that HDAC6 inhibition decreases glucose and fatty acid oxidation in T and B cells from NZB/W mice. T cells and B cells from 12-week old NZB/W female were purified and stimulated with anti CD3/CD28 or LPS respectively for 24 hours with or without the addition of 4 μM ACY-738 (DMSO only was used as control). After 24 hours of culture, CO2 production from the oxidation of glucose (FIG. 111A) and palmitate (FIG. 111B) were determined from three separate experiments in triplicate (n=3).

FIG. 112 shows a non-limiting example of results showing that HDAC6 inhibition decreases lupus gene signature pathways in NZB/W mice that are increased in active human SLE. IPA canonical signaling pathways increased in human SLE microarray tissue datasets were compared to signaling pathways in NZB/W mice decreased by the HDAC6 inhibitor. Z scores greater or less than 2 are considered significant.

FIGS. 113A-113B show a non-limiting example of quantified germinal center formation in NZB/W female mice at 24 weeks-of age-treated with ACY-738 (treated, “T”) or without ACY-738 (control, “C”) for four weeks. We randomly picked 5 germinal centers from each spleen sample and analyzed by using ImageJ software to calculate the size of the germinal center. N=20, * P<0.05, **** P<0.0001.

FIGS. 114A-114D show a non-limiting example of results obtained by flow cytometry of GC B cells (FIGS. 114A and 114C) and TFH (FIGS. 114B and 114D) assessed by flow cytometry in C57BL/6J mice and C57BL/6J/HDAC6^{-/-} mice. For spleen, n=5 (FIGS. 114A-114B), and for Peyer's patch, n=3 (FIGS. 114C-114D). Germinal center B cells are gated by CD19+, GL7+, IgD−. * P<0.05.

FIGS. 115A-115F show a non-limiting example of results obtained by flow cytometry of sorted B cells from C57BL/6J mice and C57BL/6J/HDAC6^{-/-} mice stimulated with LPS or anti-IgM, anti-CD40 for 24 hours. The results showed reduced expression of activation markers of B cells CD86 (FIG. 115A) and MHCII (FIG. 115B) in C57BL/6J/HDAC6^{-/-} mice compared to C57BL/6J mice with stimulation of anti-IgM and anti-CD40. In addition, MFI of CD69 (FIG. 115C), CD86 (FIG. 115D), MHC-II (FIG. 115E), and CD80 (FIG. 115F) are down-regulated in C57BL/6J/HDAC6^{-/-} mice with stimulation of LPS. N=5. * P<0.05, ** P<0.01

FIGS. 116A-116F show a non-limiting example of results obtained by flow cytometry of sorted B cells from NZB/W mice stimulated with LPS or anti-IgM, anti-CD40 and then treated with ACY738 for 24 hours. The results showed reduced expression of activation markers of B cells CD86 (FIG. 116A) and MHCII (FIG. 116B) in ACY-738 treated B cells with stimulation of anti-IgM and anti-CD40. In addition, MFI of CD69 (FIG. 116C), CD86 (FIG. 116D), MHC-II (FIG. 116E), and CD80 (FIG. 116F) are significantly down-regulated in ACY-738 treated B cells with stimulation of LPS. N=5. * P<0.05, ** P<0.01, *** P<0.001, **** P<0.0001.

FIGS. 117A-117C show a non-limiting example of control experiments demonstrating the specificity and lack of cross reactivity of I-scope. Experiments were performed on the DE analysis of healthy control purified CD3+CD4+ T cells (FIGS. 117A and 117C), CD19+CD3− B and Plasma Cells (FIGS. 117A-117B), and CD33+CD3− Myeloid cells (FIGS. 117B-117C) from microarray dataset GSE10325. The genes in each I-scope category (29 categories in total; hematopoietic general was not used) were used as modules for gene set variation analysis to determine the specificity of each module and cross-reactivity to other cell types. For each comparison, only categories with at least three genes above the Interquartile Range threshold were considered for statistical analysis. Significance of GSVA enrichment scores was determined using Sidak's multiple comparisons test. Adjusted p values below 0.05 were considered significant. FIGS. 117D-117E show a non-limiting example of results demonstrating a strong relationship of human B cell/microliter counts to GSVA enrichment scores for the I-scope B cell category on 105 human subjects from microarray dataset GSE88884. Demonstration of the strong relationship of mouse flow cytometry values for plasma cells (B220+IgM-CD138+) and the GSVA enrichment scores using the I-scope plasma cell module on BXSB Yaa (points above X-axis) and BXSB MPJ mice (points below X-axis).

FIG. 118 shows a non-limiting example of a process for translating mouse to human genomic data, which allows a direct comparison of human and mouse genomic data.

FIG. 119 shows a non-limiting example of a process for translating mouse to human genomic data, using a BIG-C comparison of treated mouse lupus and human lupus tissue.

FIG. 120A shows the number of differentially expressed (DE) genes detected by LIMMA analysis in MC, CD4+ T cells, and B cells isolated from inactive (SLEDAI<6) and active (SLEDAI≥6) SLE patients when compared to healthy donors. n.s.: no genes found to be significantly differentially expressed (FDR<0.2) when compared to healthy controls. FIG. 120B shows Hierarchical clustering of differentially expressed (DE) genes detected by LIMMA analysis in CD14+ MC isolated from inactive (SLEDAI<6) and active (SLEDAI≥6) SLE patients when compared to healthy donors. Arrows highlight M1 (black) or M2 (white) polarization genes. FIG. 120C shows fold change variation of genes found to be upregulated in both active and inactive SLE MC. Polarization-related genes are shown in bold and M1 genes are represented by a black wedge while M2 genes are represented with a white wedge. Genes not associated with M1 or M2 pathways are represented with a gray wedge.

FIG. 121A shows DE genes from active and inactive CD14+ MC were analyzed by GSVA to determine pathway enrichment using functional definitions provided from the BIG-C (Biologically Informed Gene Clustering) annotation library. Samples were successfully sorted by disease cohort via this method in both active and inactive MC. Starred BIG-C categories only appeared in the active or inactive analysis, respectively. FIG. 121B shows WGCNA of CD14+ and CD33+ MC isolated from SLE patients. Dendrograms show hierarchy of modules formed by unsupervised WGCNA clustering of DE genes from CD14+ and CD33+ MC isolated from active and inactive SLE patients.

FIG. 122 shows a CIRCOS diagram comparing the composition of SLE positively-correlated CD14+ and CD33+WGCNA modules to genes enriched in M1- or M2-polarized human Mϕ or genes associated with general MC activation (upregulated in both M1 and M2 conditions). Genes found in the yellow module (CD14+) are shown in black, genes found in the violet module (CD33+) are shown in red, and genes found in the sienna3 module (CD33+) are shown in orange. M1-related genes are represented with solid lines, M2-related genes are represented by dashed lines, and general MC activation genes are represented with dotted lines.

FIGS. 123A-123B show protein-protein interaction networks and clusters generated via CytoScape using the STRING and MCODE plugins. Networks were constructed of the gene lists of WGCNA modules positively (FIG. 123A, above) or negatively (FIG. 123B, below) correlated to SLEDAI from CD14⁺ MC (FIG. 123A(a) and FIG. 123B(a)) or CD33⁺ MC (FIG. 123A(b), FIG. 123A(c), FIG. 123B(b), and FIG. 123B(c)). MCODE clusters are determined by the strength of protein-protein interactions, calculated by pooling information from publicly available literature. Top half of diagrams show the cluster metastructure of each network while bottom half shows the specific genes that make up each cluster. M1-related genes are indicated by red arrows and M2-related genes are indicated by blue arrows.

FIG. 124A shows that IPA was used to analyze the CD14⁺ MC dataset and identify putative upstream regulators for active patient monocytes, inactive patient monocytes, and the active-inactive overlap using a p-value cutoff of 0.05. Only genes for which IPA assigned a z-score of ≥|2| in at least one of the three sets are shown. FIG. 124B shows representative diagrams showing downstream gene expression changes (outer circles) used to calculate upstream regulators (center).

FIG. 125 shows gene sets from CD14⁺ MC isolated from active or inactive SLE patients were used as input for the LINCS analysis platform, which reports connectivity scores for individual genes that describe how well the genomic change between the baseline and experimental input sets matches the change observed following the knockdown or overexpression of the individual gene in question. Knockdown and overexpression data were filtered by genes for which LINCS reported connectivity scores for both categories, and genes were identified as BURs for a particular dataset if they received a knockdown connectivity score between −75 and −100 and an overexpression connectivity score between 50 and 100 for that dataset.

FIG. 126A shows that GSVA was utilized to generate scores to assess enrichment of WGCNA lymphocyte subset gene modules correlated with disease activity in WB or PBMC samples separated into inactive or active SLE patients. Results are shown following unsupervised hierarchical clustering. The expected and observed correlations to disease states of each module and the cell type of their origin are shown on the right (black: positive correlation; gray: negative correlation; white: unknown correlation; x: no significant correlation). FIG. 126B shows that Odds ratios (OR) with 95% confidence intervals (CI) were calculated from the GSVA data to determine the strength of association of each cellular module with active disease. FIG. 126C shows ROC curves displaying representative results of disease activity prediction by the generalized linear model algorithm for modules from an individual cell type. Area under the curve is shown on each panel.

FIG. 127 shows PC DE profiles isolated from Published Microarray Profiles.

FIG. 128A-128C show functional characterization of DE PC gene signatures in SLE. FIG. 128A shows a filtered PC dataset containing only PC-specific gene signatures. FIG. 128B shows significantly enriched BIG-C categories found in the common DE gene signature, including ER, Golgi, Immune Cell Surface, and Unfolded Protein and Stress FIG. 128C shows that among the unique Tonsil PC DE genes, the ER, General Cell Surface, Golgi, Integrin Pathway, Secreted and ECM, and Transporters BIG-C category ORs were significantly enriched while the Endocytosis, Mitochondrial DNA-to-RNA, Mitochondria General, mRNA Splicing, mRNA Translation, Nuclear Hormone Receptors, and Nucleus and Nucleolus BIG-C categories were significantly underrepresented.

FIG. 129A-129B show protein interaction-based clustering of SLE PC and SLE/Tonsil Common DE genes. FIG. 129A shows that DE genes common to the SLE PC and Tonsil PC datasets formed four discrete clusters: a large unfolded protein response/secreted protein cluster, an ER cluster, a small unfolded protein response cluster, and a small cluster with undefined function. FIG. 129B shows that the SLE PC DE list produced only two clusters via MCODE analysis: one large cluster centered around pro-proliferation signaling pathways, and one small cluster containing ER- and mitochondria-related genes.

FIGS. 130A-130B show results of tracking a PC DE signature in the periphery and tissues of SLE patient via microarray data. FIG. 130A shows that many of the genes were found to be upregulated most in the skin and synovium, followed by the kidney and B cell datasets, with some expression detected in the PBMC and WB datasets. FIG. 130B shows that using the SLE PC and Common PC DE gene lists revealed enrichment patterns of divergent subsets of the PC signature across different SLE tissue and peripheral cell datasets.

FIGS. 131A-131E show that GSVA was used to determine enrichment of the Tonsil PC, SLE PC, and Common signatures in tissue (FIG. 131A-131D) and PBMC samples (FIG. 131E) from SLE, DLE, LN, and OA patients. FIG. 131A-131C show that enrichment of the Common and SLE PC signatures only appeared to successfully identify and sort DLE, SLE, and LN patient samples in the skin, synovium, and kidney glomerulus, respectively. FIG. 131D shows that LN patient samples were less cleanly identified from healthy control samples when these signatures were applied to the kidney tubulointerstitium, but the Common signature tended to be enriched in LN patient samples while the Tonsil PC signature (representing homeostatic/healthy PC gene signaling) tended to be enriched in the control samples. FIG. 131E shows that PBMC samples were not successfully discriminated by cohort according to GSVA enrichment of the Tonsil PC/SLE PC/Common signature paradigm.

FIGS. 132A-132C show identifying targets of the proteasome inhibitor family of chemotherapy agents (bortezomib, ixazomib, carfilzomib) as members and regulators of the SLE PC signature by multiple methods, including analysis of upstream regulators of SLE PC DE gene signatures cluster in proliferation and cell cycle checkpoint pathways. IPA upstream regulator analysis was used to further distill the SLE PC DE signature and identify keystone genes and signaling pathways. High-priority targets were generated via IPA upstream regulator analysis (FIG. 132A) and by cross-reference with the AMPEL Primary Immunodeficiency Gene Database (FIG. 132B), which identifies and catalogs keystone genes that act as checkpoints in the development of autoimmunity and protect against gross failure of immune tolerance.

FIG. 133A-133D show results obtained by mapping the functional genes predicted by SLE-associated SNPs. FIG. 133A shows a distribution of genomic functional categories for ancestry-specific non-HLA associated SLE SNPs (Tiers 1-3). Non-coding regions include micro (mi)RNAs, long non-coding (lnc)RNAs, introns and intergenic regions. Regulatory regions include transcription factor binding sites (TFBS), promoters, enhancers, repressors, promoter flanking regions and open chromatin. Coding regions were broken down further and include 5′UTRs, 3′UTRs, synonymous and nonsynonymous (missense and nonsense) mutations. FIG. 133B shows that functional genes predicted by SNPs are derived from 4 sources including regulatory elements (T-Genes), eQTL analysis (E-Genes), coding regions (C-Genes) and proximal gene-SNP annotation (P-Genes). FIG. 133C shows a Venn diagram depicting the overlap of all SLE-associated SNPs. FIG. 133D shows a Venn diagram depicting the overlap of and all predicted E-, T-, P-, and C-Genes.

FIGS. 134A-134E show the characterization of predicted gene signatures. FIG. 134A shows that ancestry-dependent and independent E-, P-, T-, and C-Genes were analyzed to determine enrichment using functional definitions from the BIG-C (Biologically Informed Gene Clustering) annotation library. Enrichment was defined as any category with an odds ratio (OR)>1 and −log 10(p-value)>1.33. FIGS. 134B-134E shows heatmap visualizations of the top five significant IPA canonical pathways for each gene list (E-, P-, T-Genes) organized by ancestry. C-Genes were analyzed together. Top pathways with −log 10(p-value)>1.33 are listed.

FIGS. 135A-135D show that cluster metastructures were generated based on PPI networks, clustered using MCODE and visualized in CytoScape. Size indicates the number of genes per cluster, edge weight indicates the number of inter-cluster connections and color indicates the number of intra-cluster connections. FIG. 135E shows the quantitation of cluster size, intra- and intercluster connections. Error bars represent the 95% confidence interval; asterisks (*) indicate a p-value<0.05 using Welch's t-test.

FIG. 136A-136C shows that ancestry-specific E-, P-, T-, and C-Genes were matched to differential expression (DE) SLE datasets in various tissues, including whole blood, PBMCs, B-cells, T-cells, synovium, skin and kidney.

FIGS. 137A-137B show that DE predicted genes and UPRs were used as input to build STRING-based PPI networks, visualized in CytoScape, and clustered with MCODE. Individual clusters were then analyzed by BIG-C and IPA to identify those molecules and pathways highly associated with disease. A total of 45 pathways were representative of EA DE genes and UPRs, with the largest clusters 3 and 1 heavily involved in pattern recognition receptor signaling (activation of IRFs by cytosolic PRRs and role of RIG-I in antiviral immunity).

FIGS. 138A-138B show that the AA network was smaller (FIG. 138A), containing fewer predicted genes and associated UPRs, yet shared multiple pathways with EA, including B cell receptor signaling, GPCR signaling, opioid signaling, phagocyte maturation and hepatic cholestasis, a pathway involved in bile acid synthesis (FIG. 138B).

FIGS. 139A-139B show that pathways exemplified by ancestry-independent genes were a blend of both EA and AA pathways. For example, common pathways included IL12 signaling and production by macrophages, TLR signaling and activation of IRFs by cytosolic PRRs, pathways that were predicted by EA genes and UPRs, as well as PRRs in the recognition of bacteria and virus, a pathway shared with AA.

FIGS. 140A-140F depict both the unique and overlapping canonical pathways predicted by the EA and AA gene sets. Examination of pathway categories shared between EA and AA ancestral groups are those commonly associated with SLE representing aberrant immune function, altered transcriptional regulation, and abnormal cell cycle control, providing additional confirmation for the global gene expression analysis presented here (FIG. 140B).

FIGS. 141A-141C show an overview of gene expression in SLE vs OA synovium. FIG. 141A shows that DE analysis was conducted on gene expression data from SLE and OA synovium resulting in 6,496 DE genes, 2,477 upregulated in SLE and 4,019 downregulated in SLE. FIG. 141B shows that increased and decreased transcripts were each characterized by I-Scope and T-Scope (fibroblasts, synoviocytes) for prevalence of specific cell types. FIG. 141C shows that DE transcripts were also characterized by BIG-C for functional enrichment. Heatmaps in FIGS. 141B-141C shows that the figures represent the negative logarithm of the overlap p-value when odds ratio is greater than 1 by Fisher's Exact Test. Gray cells represent non-significant enrichment (p>0.05 or OR>1). A minimum p-value of 2.2e-16 was used.

FIGS. 142A-142C show that WGCNA reveals SLE-associated modules of genes enriched in immune cells. WGCNA of 4 SLE vs 4 OA patients yielded 7 modules of genes associated with SLE after QC and were characterized by I-Scope, T-Scope, and BIG-C. FIG. 142A shows module eigengene plots per sample of the 7 SLE-associated modules; color names are randomly generated as part of WGCNA module assignment. FIG. 142B shows that the negative logarithms of the overlap p-values identify specific immune/inflammatory cell populations or synovium-specific cell populations that may be linked to lupus synovitis or to indicate enrichment of functional gene categories (FIG. 142C). Data shown in FIGS. 142B-142C shows that the figures are significant (p<0.05) by right-sided Fisher's Exact test and must have an odds ratio above 1 to indicate enrichment.

FIGS. 143A-143B show signaling pathways and upstream regulators operative in lupus synovitis. IPA canonical pathway and upstream regulator analysis was performed. FIG. 143A shows consensus canonical pathways predicted to be significantly activated or inhibited by DE transcripts and at least one SLE-associated WGCNA module. FIG. 143B shows that consensus upstream regulators predicted to be significantly activated or inhibited by both DE transcripts and at least one SLE-associated WGCNA module are displayed and organized by BIG-C category. Canonical pathways and upstream regulators were considered significant if |Activation Z-Score|≥2 and overlap p-value≤0.01.

FIG. 144 shows germinal center B cell and Tfh cell markers in lupus synovitis, including an assessment of germinal center and follicular T helper cell markers in lupus synovium from DE genes or WGCNA. Genes found in SLE-associated WGCNA modules are indicated.

FIG. 145 shows that GSVA enrichment of immune populations in synovia confirms inflammatory infiltrate in SLE. GSVA of relevant immune cell populations, molecular signatures, and signaling pathways was conducted on log 2-normalized gene expression values from OA and SLE synovia. Significant differences in enrichment between cohorts were found by Welch's t-test (*p<0.05). Hedge's g effect sizes were calculated (right) with correction for small sample size for each gene set; zeroes represent non-significant differences in enrichment between cohorts. “#” indicates a literature-derived signature. Other gene set signatures were derived from IPA, where noted, PathCards, or are hand-curated lists from lupus gene expression data and literature mining.

FIG. 146 shows LINCS biological upstream regulators, including the top 50 targets from LINCS knockdown and overexpression data matching (overexpressed) and opposing (knocked down) the lupus synovitis gene signature. Knockdown and overexpression data were analyzed for connectivity scores in the −75 to −100 and 50 to 100 ranges, respectively. Drugs and compounds directly or indirectly antagonizing/inhibiting the biological upstream regulators were sourced from LINCS/CLUE, IPA®, literature mining, CoLTS, STITCH, and clinical trials databases. Where applicable, drug annotations are grouped together by target and CoLTS scores are displayed as integers in superscript. Indirect drug matches are displayed in italics. Only drugs with CoLTS scores are shown. “P”: Preclinical; “‡”: Drug in development/clinical trials; “†”: FDA-approved.

FIGS. 147A-147B show a comparison of gene expression between SLE and RA synovitis. A comparison of immune/inflammatory and synovial gene signatures was made between SLE and RA synovium using 7 RA patients from GSE36700. FIG. 147A shows that upregulated DEGs were identified between RA and OA synovium, compared to SLE, and characterized by I-Scope. FIG. 147B shows that GSVA of immune/inflammatory cell populations, molecular signatures, and signaling pathways was carried out on log 2-normalized gene expression values from RA and SLE synovia. Significant differences in enrichment between cohorts were found by Welch's t-test (*p<0.05). Hedge's g effect sizes were calculated (right) with correction for small sample size for each gene set; zeroes represent non-significant differences in enrichment between cohorts. “#” indicates a literature-derived signature. Other gene set signatures were derived from IPA, where noted, PathCards, or are hand-curated lists from lupus gene expression data and literature mining.

FIG. 148 shows a model of lupus synovitis. DEGs, molecules co-expressed in SLE correlated WGCNA modules, and IPA® upstream regulator predictions were integrated into a summary model of lupus synovitis. Transcripts listed on the right were either upregulated (red text), co-expressed in SLE correlated WGCNA modules (underlined), or identified as upstream regulators operative in lupus synovitis.

FIG. 149 shows an example of weighted gene co-expression network analysis (WGCNA) to create modules of correlated genes through hierarchical clustering, including constructing a gene co-expression network by gene:gene correlations across samples, identifying co-expression modules by dynamic cutting of hierarchical clustering trees, and correlating module eigengenes with phenotypic information.

FIGS. 150A-150C show that WGCNA identified modules with significant correlations to clinical variables in DLE datasets. WGCNA identified 41 modules for GSE72535, 23 modules for GSE81071, and 30 modules for GSE52471. FIG. 150A shows that in GSE72535, 12 modules were significantly correlated to CLASI.A or cohort (5 positively and 7 negatively).

FIGS. 150B-150C show that in GSE81071 (FIG. 150B) and (FIG. 150C) GSE52471, 7 modules were significantly correlated to cohort (GSE81071: 4 positively and 3 negatively; GSE52471: 2 positively and 5 negatively).

FIGS. 151A-151B show WGCNA modules interrogated using BIG-C® functional characterizations as well as I-Scope™ and T-Scope™ for specific cellular subsets. DLE-associated modules identified in WGCNA are characterized by BIG-C® (FIG. 151A) and I-Scope™/T-Scope™ (FIG. 151B). Odds ratios above 1 are shown, and Fisher's exact tests with p-values below 0.05 are indicated with an asterisk. Consistent enrichment of several categories, including immune signaling, pattern recognition receptors, and pro-apoptosis, was seen across all three analyses. Additionally, a clear immune signature, including antigen presenting cells, T cells, and myeloid cells, was observed in positively correlated modules.

FIG. 152 shows WGCNA modules statistically preserved and common DE genes between three analyses. Module preservation was performed for each pairwise combination of datasets. The preservation Zsummary statistic was used to determine significant preservation. A representative example of the WGCNA modules from GSE81071 in the preservation analysis between GSE81071 and GSE52471. The overlap p-value (Fisher's exact test) was used to determine specific module associations between datasets. Interestingly, the analyses consistently showed the preservation of the two positively correlated modules in each dataset (Turquoise and Plum2 in GSE72535, Brown and Magenta in GSE81071, and Blue and LightGreen in GSE52471).

FIG. 153 shows BIG-C®, I-Scope™ and T-Scope™ analysis results in the preserved modules and common DE genes. The analysis compared DE genes common to all three datasets and the 6 preserved DLE-associated WGCNA modules. BIG-C® (left) and I-Scope or T-scope categories (right) found to have an odds ratios above 1 in both DE transcripts and at least one module from each dataset are shown. Fisher's exact tests with p-values below 0.05 are indicated with an asterisk.

FIGS. 154A-154B show results of IPA® canonical pathway and upstream regulator (UR) analysis. IPA® canonical pathway and upstream regulator analysis was performed. The analysis compared DE genes common to all three datasets and the 6 preserved DLE-associated WGCNA modules. FIG. 154A shows canonical pathways predicted to be significantly activated or inhibited in both DE transcripts and at least one module from each dataset. FIG. 154B shows that a total of 224 URs were significantly activated or inhibited in both the DE transcripts and at least one module from each dataset. The 84 URs targeted by existing drugs are shown and organized by BIG-C™ category. Canonical pathways and upstream regulators were considered significant if |Activation Z-Score|≥2.

### DETAILED DESCRIPTION

### Analysis by Molecular Endotyping

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” refers to an amount that is near the stated amount by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, the term “Gini impurity” refers to a measure of how often a randomly chosen element from the set may be incorrectly labeled if it is randomly labeled according to the distribution of labels in the subset.

Many complex and multi-systematic diseases and conditions currently pose major diagnostic and therapeutic challenges. Despite the wealth of records from, for example, genetic, epigenetic, and gene expression data that has emerged in the past few years, physicians often still rely on clinical evaluation and laboratory tests, including measurement of autoantibodies and complement levels.

Successful relation of records (e.g., gene expression records) to a specific disease phenotype activity has been attempted, including efforts to identify individual genes that predicted subsequent flares, and through the determination of a discrete group of differentially expressed (DE) genes that may be found in a particular record. Despite these advances, however, no such approach is available with sufficient predictive value to utilize in evaluation and treatment.

As such, there is a need for a predictive tool for evaluating patient at both the chemical and cellular levels to advance personalized treatment. Data analytical techniques such as machine learning enable proper correlation between genetic records and phenotypes.

The machine learning models tested here provide the basis of personalized medicine. Integration of the methods herein with emerging high-throughput record sampling technologies may unlock the potential to develop a simple blood test to predict phenotypic activity. The disclosures herein may be generalized to predict other manifestations, such as organ involvement. A better understanding of the cellular processes that drive pathogenesis may eventually lead to customized therapeutic strategies based on records' unique patterns of cellular activation.

### Method of Identifying One or More Records Having a Specific Phenotype

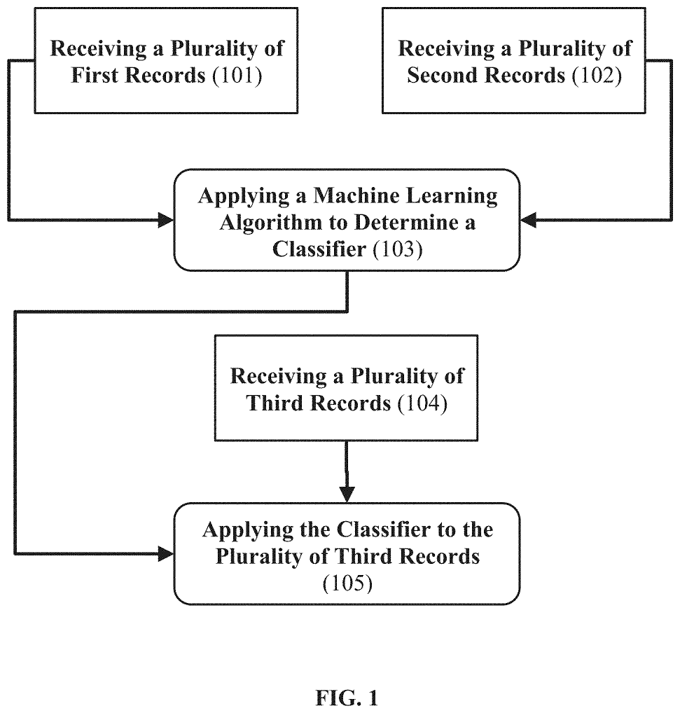

One aspect disclosed herein, per FIG. 1, is a method of identifying one or more records (e.g., raw gene expression data, whole gene expression data, blood gene expression data, or informative gene modules). The method may comprise receiving a plurality of first records 101, receiving a plurality of second records 102, receiving a plurality of third records 104, applying a machine learning algorithm to at least one first record and at least one second record to determine a classifier (e.g., a machine learning classifier) 103, and applying the classifier to the plurality of third records 105. Applying the classifier to the plurality of third records 105 may identify one or more third records associated with the specific phenotype. In some embodiments, applying a machine learning algorithm to the third data set 105 comprises applying a machine learning algorithm to a plurality of unique third data sets.

### Records

The records may comprise, for example, raw gene expression data, whole gene expression data, blood gene expression data, informative gene modules, or any combination thereof. The records may be generated by Weighted Gene Co-expression Network Analysis (WGCNA). In some embodiments, at least one of the first records and the second records comprise nucleic acid sequencing data, transcriptome data, genome data, epigenome data, proteome data, metabolome data, virome data, metabolome data, methylome data, lipidomic data, lineage-ome data, nucleosomal occupancy data, a genetic variant, a gene fusion, an insertion or deletion (indel), or any combination thereof. In some embodiments, the first records and the second records are in different formats. In some embodiments, the first records and the second records are from different sources, different studies, or both.

In some embodiments each record is associated with a specific phenotype (e.g., a disease state, an organ involvement, or a medication response). Each first record may be associated with one or more of a plurality of phenotypes. The plurality of second records and the plurality of first records may be non-overlapping. The third records may be distinct from the plurality of first records, the plurality of second records, or both. The third records may comprise a plurality of unique third data sets.

The records may be received from the Gene Expression Omnibus. The records may be associated with purified cell populations, whole blood gene expression, or both. The raw Gene Expression Omnibus source may comprise GSE10325 (e.g., from www.ncbi.nlm.nih.gov/geo/query/acc.cgi?acc=GSE10325), GSE26975 (e.g., from www.ncbi.nlm.nih.gov/geo/query/acc.cgi?acc=GSE26975), GSE38351 (e.g., from www.ncbi.nlm.nih.gov/geo/query/acc.cgi?acc=GSE38351), GSE39088 (e.g., from www.ncbi.nlm.nih.gov/geo/query/acc.cgi?acc=GSE39088), GSE45291 (e.g., from www.ncbi.nlm.nih.gov/geo/query/acc.cgi?acc=GSE45291), GSE49454 (e.g., from www.ncbi.nlm.nih.gov/geo/query/acc.cgi?acc=GSE49454), or any combination thereof.

For example, as the most important genes may be involved in a number of functions other than interferon signaling, such RNA processing, ubiquitylation, and mitochondrial processes, these pathways may play important roles in directing, or at least be indicative of, phenotypic activity. CD4 T cells originally may contribute the most important modules. However, when the modules are de-duplicated, CD14 monocyte-derived modules prove important as unique genes expressed by CD14 monocytes in tandem with interferon genes may be informative in the study of cell-specific methods of pathogenesis.

### Phenotypes

In some embodiments, the phenotype comprises a disease state, an organ involvement a medication response, or any combination thereof. The disease state may comprise an active disease state, or an inactive disease state. At least one of the active disease state and the inactive disease state may be characterized by standard clinical composite outcome measures. The active disease state may comprise a Disease Activity Index of 6 or greater.

The disease may comprise an acute disease, a chronic disease, a clinical disease, a flare-up disease, a progressive disease, a refractory disease, a subclinical disease, or a terminal disease. The disease may comprise a localized disease, a disseminated disease, or a systemic disease. The disease may comprise an immune disease, a cancer, a genetic disease, a metabolic disease, an endocrine disease, a neurological disease, a musculoskeletal disease, or a psychiatric disease. The active disease state may comprise a Systemic Lupus Erythematosus Disease Activity Index (SLEDAI) of 6 or greater.

The organ involvement may comprise a possibly involved organ. The possibly involved organ may comprise bone, skin, hematopoietic system, spleen, liver, lung, mucosa, eye, ear, pituitary, or any combination thereof. The medication response may comprise an ultra-rapid metabolizer response, an extensive metabolizer response, an intermediate metabolizer response, or a poor metabolizer response. The ultra-rapid metabolizer response may refer to a record with substantially increased metabolic activity. The extensive metabolizer response may refer to a record with normal metabolic activity. The intermediate metabolizer response may refer to a record with reduced metabolic activity. The poor metabolizer response may refer to a record with little to no functional metabolic activity.

### Machine Learning and Classifiers

The classifiers described herein may be used in machine learning algorithms. A variety of machine learning classifiers exist, wherein each classifier produces a unique machine learning process and/or output. The machine learning algorithms may comprise a biased algorithm or an unbiased algorithm. The biased algorithm may comprise Gene Set Enrichment Analysis (GSVA) enrichment of phenotype-associated cell-specific modules. The unbiased approach may employ all available phenotypic data. The machine learning algorithm may comprise an elastic generalized linear model (GLM), a k-nearest neighbors classifier (KNN), a random forest (RF) classifier, or any combination thereof. GLM, KNN, and RF machine learning algorithms may be performed using the glmnet, caret, and randomForest R packages, respectively.

The random forest classifier is able to sort through the inherent heterogeneity of the plurality of records to identify one or more third records associated with the specific phenotype. In some embodiments, the classifier identifies said one or more third records associated with the specific phenotype with an accuracy of at least about 70%. The implementation of the random forest classifier herein enable a specific phenotype association sensitivity of 85% and a specific phenotype association specificity of 83%. Further classifier optimization, however, may yield improved results.

KNN may classify unknown samples based on their proximity to a set number K of known samples. K may be 5% of the size of the pluralities of first, second, and third records. Alternatively, K may be 1%, 2%, 3%, 4%, 5%, 6%, 7%, 8%, 9%, 10%, or any increment therein. A large K value may enable more precise calculations with less overall noise. Alternatively, the k-value may be determined through cross-validation by using an independent set of records to validate the K value. If the initial value of k is even, 1 may be added in order to avoid ties. RF may generate 500 decision trees which vote on the class of each sample. The Gini impurity index, a standard measure of misclassification error, correlates to the importance of such variables. In addition, pooled predictions may be assigned based on the average class probabilities across the three classifiers.

The GLM algorithm may carry out logistic regression with a tunable elastic penalty term to find a balance between an L1 (LASSO) and an L2 (ridge), whereby penalties facilitate variable selection in order to generate sparse solutions. Least Absolute Shrinkage and Selection Operator (LASSO) is a regularization feature selection technique to reduce overfitting in regression problems. Ridge regression employs a penalty term is to shrink the LASSO coefficient values. In some embodiments, the elastic generalized linear model classifier employs an elastic penalty of about 0.9, wherein the penalty is 90% lasso and 10% ridge. The elastic penalty may be 0.1, 0.2, 0.3, 0.4, 0.5, 0.6, 0.7, 0.8, 0.9, or any increments therein.

Records may be classified as active or inactive using two different methodologies: (1) a leave-one-study-out cross-validation approach or (2) a 10-fold cross-validation approach. GLM, KNN, and RF classifiers may be tasked with identifying active and inactive state records based on whole blood (WB) gene expression data and module enrichment data.

Supervised classification approaches using elastic generalized linear modeling, k-nearest neighbors, and random forest classifiers may be implemented. The trends in performance when cross-validating by one of the pluralities of records or cross-validating 10-fold display the potential advantages and disadvantages of diagnostic tests incorporating gene expression data or module enrichment. Cross-validating by one of the pluralities of records may be used to generalize 1-fold cross validation as a suboptimal scenario, whereas a 10-fold cross-validation is in fact more optimal. Although classification of active and inactive records from the pluralities of different records with 1-fold cross-validation may be suboptimal, module enrichment may be employed to smooth out much of the technical variation between data sets. 10-fold cross-validation may enable a more standardized diagnostic test. Although the plurality of second records and the plurality of first records are non-overlapping, the test set employs overlapping records to facilitate proper classification.

Furthermore, modules that may be negatively associated with phenotypic activity may be just as important in classification as positively associated modules. Further study of underrepresented categories of transcripts may enhance understanding and correlation of phenotypic activity.

Reduction of technical noise may improve classification. For example, RNA-Seq platforms, which produce transcript count records rather than probe intensity values, may display less technical variation across records if all samples are processed in the same way.

The strong performance of the random forest classifier indicates that nonlinear, decision tree-based methods of classification may be ideal because decision trees ask questions about new records sequentially and adaptively. Random forest does not apply a one-size-fits-all approach to each of the different types of records to allow for classification of records whose expression patterns make them a minority within their phenotype. As such, active records that do not resemble the majority of active records still have a strong chance of being properly classified by random forest. By contrast other methods may approach variables from new records all at once.

### Filtering

In some embodiments, the method further comprises filtering the first records, the second records, or both. In some embodiments, the filtering comprises normalizing, variance correction, removing outliers, removing background noise, removing data without annotation data, scaling, Weighted Gene Co-expression Network Analysis, enrichment analysis, dimensionality reduction, or any combination thereof.

In some embodiments, the normalizing is performed by Robust Multi-Array Analysis (RMA), Guanine Cytosine Robust Multi-Array Analysis (GCRMA), Linear Models for Microarray Data, variance stabilizing transformation (VST), normal-exponential quantile correction (NEQC), or any combination thereof. RMA may summarize the perfect matches through a median polish algorithm, quantile normalization, or both. Variance-stabilizing transformation may simplify considerations in graphical exploratory data analysis, allow the application of simple regression-based or analysis of variance techniques, or both. Normalized expression values may be variance corrected using local empirical Bayesian shrinkage, and DE may be assessed using the Linear Models for Microarray Data (LIMMA) package. Resulting p-values may be adjusted for multiple hypothesis testing using the Benjamini-Hochberg correction, which resulted in a false discovery rate (FDR). Significant genes within each study may be filtered to retain DE genes with an FDR<0.2, which may be considered statistically significant. The FDR may be selected a priori to diminish the number of genes that may be excluded as false negatives.

In some embodiments, the variance correction comprises employing a local empirical Bayesian shrinkage, adjusting the p-values for multiple hypothesis testing using the Benjamini-Hochberg correction, removing all data with a false discovery rate of less than 0.2, or any combination thereof. The Benjamini-Hochberg procedure may decrease the false discovery rate caused by incorrectly rejecting the true null hypotheses control for small p-values.

In some embodiments, the Weighted Gene Co-expression Network Analysis comprises calculating a topology matrix, clustering the data based on the topology matrix, correlating module eigenvalues for traits on a linear scale by Pearson correlation for nonparametric traits by Spearman correlation and for dichotomous traits by point-biserial correlation or t-test, or both. A topology matrix may specify the connections between vertices in directed multigraph.

Log 2-normalized microarray expression values from purified CD4, CD14, CD19, CD33, and low density granulocyte (LDG) populations may be used as input to WGCNA to conduct an unsupervised clustering analysis, resulting in co-expression “modules,” or groups of densely interconnected genes which may correspond to comparably regulated biologic pathways. For each experiment, an approximately scale-free topology matrix (TOM) may be first calculated to encode the network strength between probes. Probes may be clustered into WGCNA modules based on TOM distances. Resultant dendrograms of correlation networks may be trimmed to isolate individual modular groups of probes by partitioning around medoids and labeled using color assignments based on module size. Expression profiles of genes within modules may be summarized by a module eigengene (ME), which may be analogous to the module's first principal component. MEs act as characteristic expression values for their respective modules and may be correlated with sample traits such as SLEDAI or cell type by Pearson correlation for continuous or semi-continuous traits and by point-biserial correlation for dichotomous traits.

WGCNA modules from CD4, CD14, CD19, and CD33 cells may be tested for correlation to SLEDAI. Plasma cell modules may be generated by differential expression analysis and not WGCNA, but may be included because of the established importance of plasma cells in SLE pathogenesis.

Removing the outliers may be performed by statistical analysis using R and relevant Bioconductor packages. Non-normalized arrays may be inspected for visual artifacts or poor hybridization using Affy QC plots. Principal Component Analysis (PCA) plots may be used to inspect the raw data files for outliers. Data sets culled of outliers may be cleaned of background noise and normalized using RMA, GCRMA, or NEQC where appropriate. Data sets may be then filtered to remove probes with low intensity values and probes without gene annotation data. WB gene expression data sets may be filtered to only include genes that passed quality control in all data sets. Differential expression (DE) analysis and WGCNA may then be carried out on data sets. WB gene expression data sets may then be further processed before machine learning analysis. WB gene expression values may be centered and scaled to have zero-mean and unit-variance within each data set and the standardized expression values from each data set may be joined for classification.

The GSVA-R package may be used as a non-parametric method for estimating the variation of pre-defined gene sets in WB gene expression data sets. Standardized expression values from WB data sets may be used to test for enrichment of cell-specific WGCNA gene modules using the Single-sample Gene Set Enrichment Analysis (ssGSEA) method, which scores single samples in isolation and may be thus shielded from technical variation within and among data sets. Statistical analysis of GSVA enrichment scores may be performed by Spearman correlation or Welch's unequal variances t-test, where appropriate. GSVA may be performed on three WB datasets using 25 WGCNA modules made from purified cells with correlation or published relationship to SLEDAI (Table 1).

Patterns of enrichment of WGCNA modules that are derived from isolated cell populations of WB that are correlated to the phenotype may be more useful than gene expression across the pluralities of records to identify active versus inactive state records. To characterize the relationships between gene signatures from various records and phenotypic activity, WGCNA may be used to generate co-expression gene modules from purified populations of cells from records with an active disease state. Such records may be subsequently tested for enrichment in whole blood of other records. WGCNA analysis of leukocyte subsets may result in several gene modules with significant Pearson correlations to SLEDAI (all |r|>0.47, p<0.05). CD4, CD14, CD19, and CD33 cells with 3, 6, 8, and 4 significant modules, respectively (Table 1). Two low-density granulocyte (LDG) modules may be created by performing WGCNA analysis of LDGs along with either neutrophils or HC neutrophils and merging the modules most strongly expressed by LDGs Two plasma cell (PC) modules may be created by using the most increased and decreased transcripts of isolated plasma cells compared to naïve and memory B cells.

TABLE 1
Gene modules identified as correlating with SLEDAI via WGCNA analysis of leukocytes

| | | Module | Correlation with | | |
|---|---|---|---|---|---|
| Cell Type | Module Name | Size | SLEDAI | Top GO Biological Process | Top BIG-C Category |
| CD4 | Floralwhite | 237 | 0.81 | type I interferon signaling pathway | Interferon-Stimulated-Genes |
| | Turquoise | 805 | 0.50 | positive reg of ubiquitin-protein ligase | Proteasome |
| | Orangered4 | 237 | −0.77 | translational initiation | mRNA-Translation |
| CD14 | Plum1 | 247 | 0.47 | ubiquitin-dependent protein catabolic process | mRNA-Translation |
| | Yellow | 356 | 0.65 | type I interferon signaling pathway | Interferon-Stimulated-Genes |
| | Greenyellow | 89 | −0.49 | transcription from RNA polymerase II promoter | General-Transcription |
| | Pink | 261 | −0.77 | protein phosphorylation | Endosome-and-Vesicles |
| | Purple | 124 | −0.66 | inositol phosphate metabolic process | Fatty-Acid-Biosynthesis |
| | Sienna3 | 222 | −0.64 | translational initiation | mRNA-Translation |
| CD19 | Darkolivegreen | 591 | 0.78 | cell division | Proteasome |
| | Greenyellow | 251 | 0.66 | Notch signaling pathway | mRNA-Translation |
| | Steelblue | 146 | 0.65 | gluconeogenesis | Glycolysis-Gluconeogenesis |
| | Turquoise | 572 | 0.50 | ER to Golgi vesicle-mediated transport | Unfolded-Protein-and-Stress |
| | Violet | 566 | 0.61 | mitochondrial respiratory chain complex I | Interferon-Stimulated-Genes |
| | Brown | 620 | −0.62 | regulation of transcription, DNA-templated | Chromatin-Remodeling |
| | Green | 541 | −0.49 | transcription, DNA-templated | Transcription-Factors |
| | Skyblue | 755 | −0.74 | viral transcription | mRNA-Translation |
| CD33 | Royalblue | 94 | 0.60 | positive reg of cytosolic calcium ions | Transposon-Control |
| | Sienna3 | 133 | 0.76 | type I interferon signaling pathway | Interferon-Stimulated-Genes |
| | Violet | 177 | 0.79 | defense response to virus | Interferon-Stimulated-Genes |
| | Darkmagenta | 273 | −0.49 | ubiquinone biosynthetic process | MHC-Class-TWO |
| LDG⁺ | LDG_A | 334 | 0.79 | platelet degranulation | Cytoskeleton |
| | LDG_B | 92 | 0.81 | regulation of transcription | Secreted-Immune |
| PC* | PC_Up | 423 | N/A | protein N-linked glycosylation | Endoplasmic-Reticulum |
| | PC_Down | 183 | N/A | antigen processing and presentation MHC II | MHC-Class-TWO |

Gene Ontology (GO) analysis of the genes within each of the record indicates that that some processes, such as those related to interferon signaling, RNA transcription, and protein translation, may be shared among cell types, whereas other processes may be unique to certain cell types (Table 1) and may be used to better classification of records.

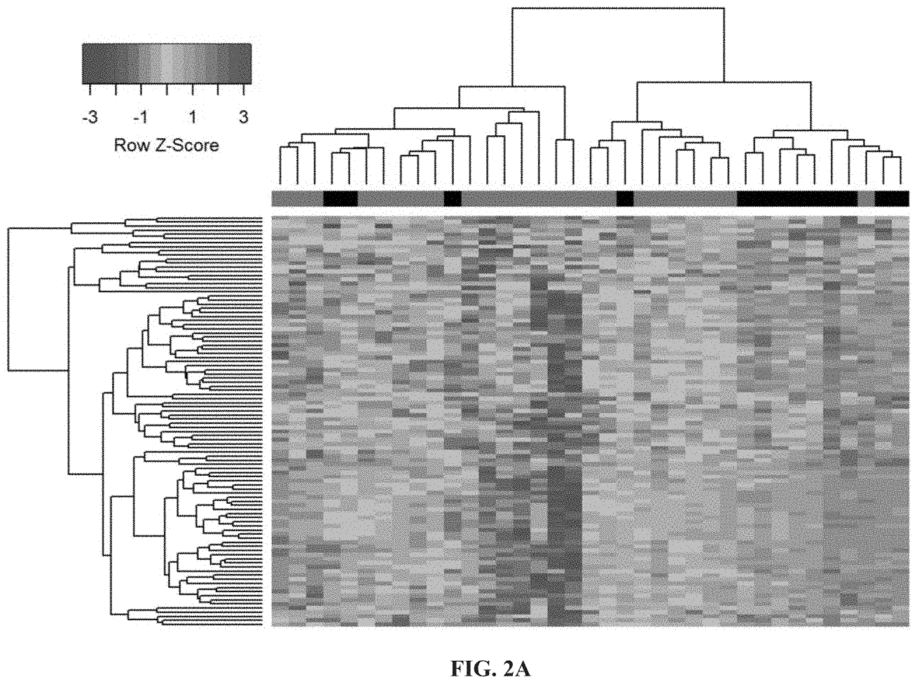

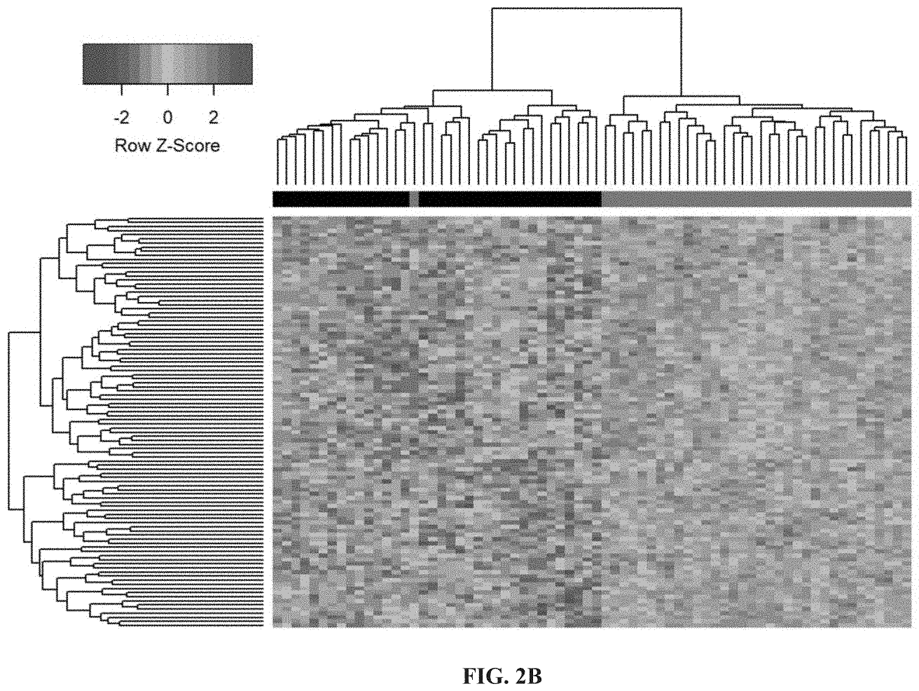

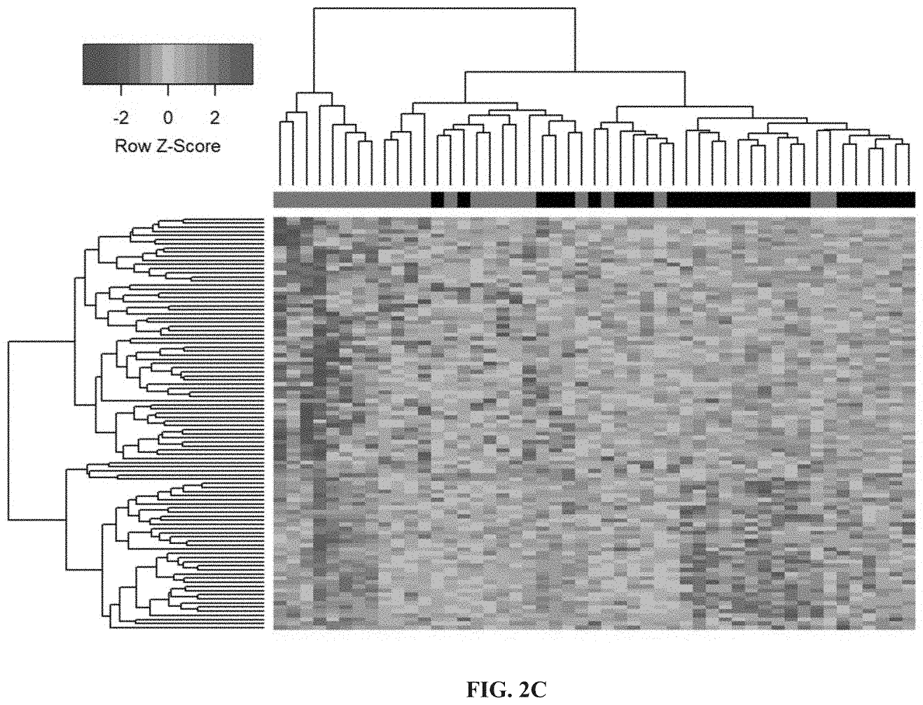

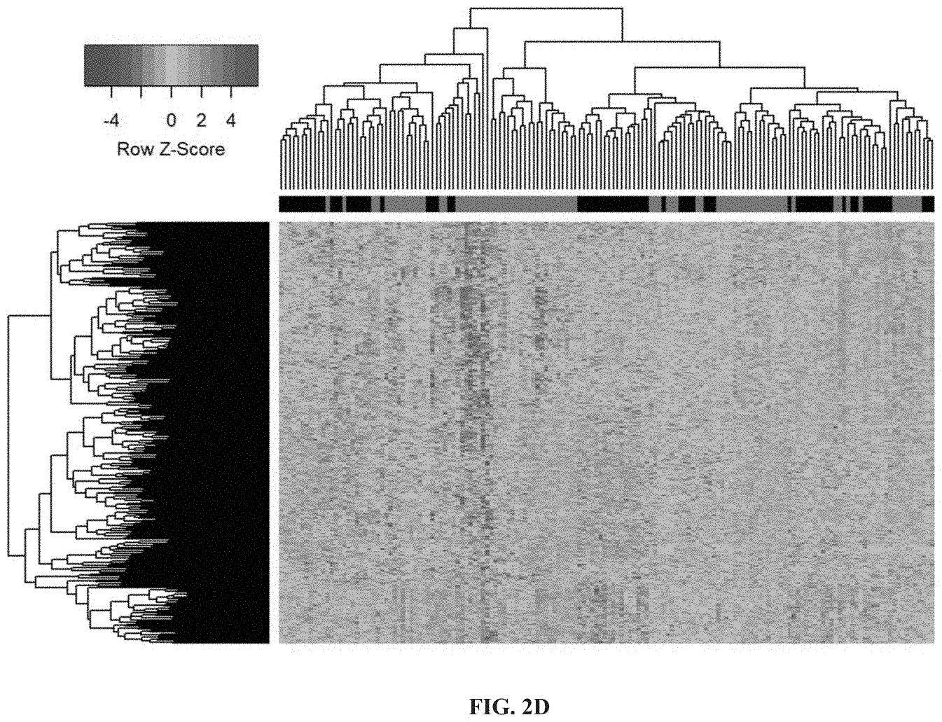

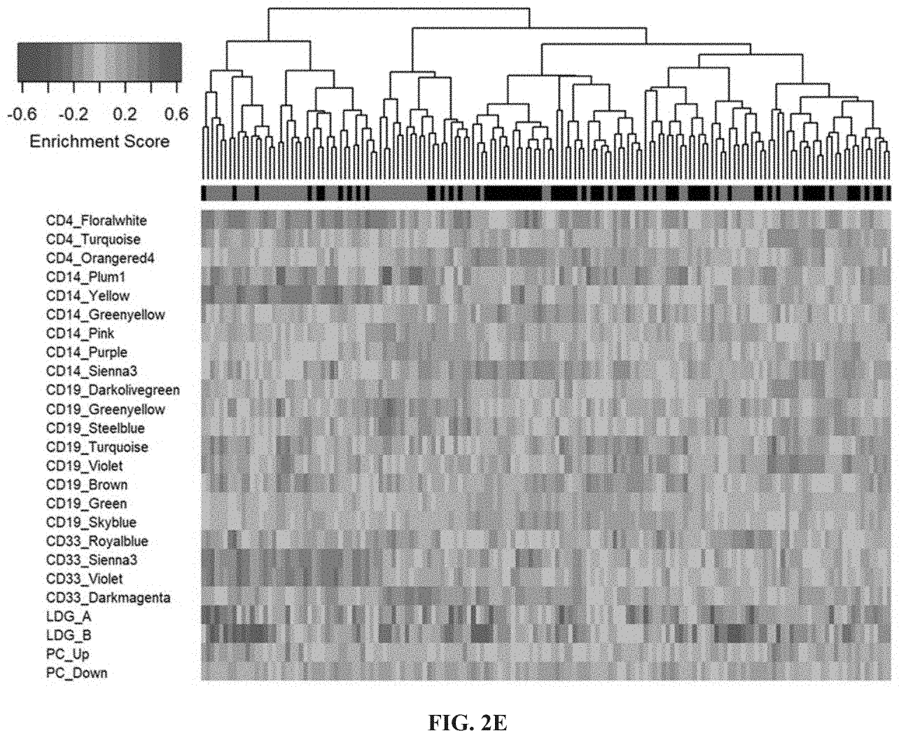

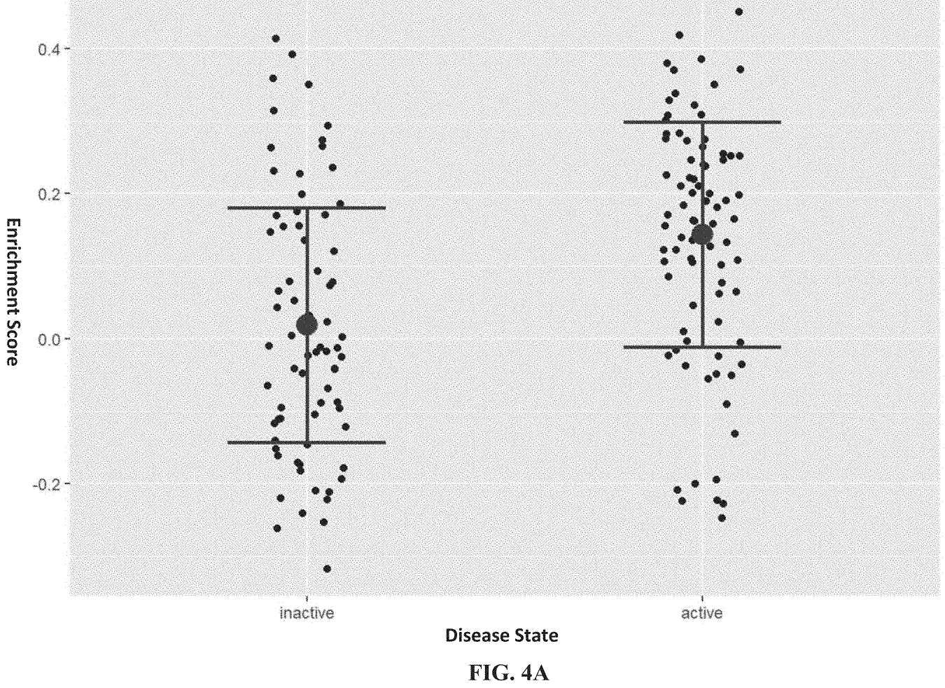

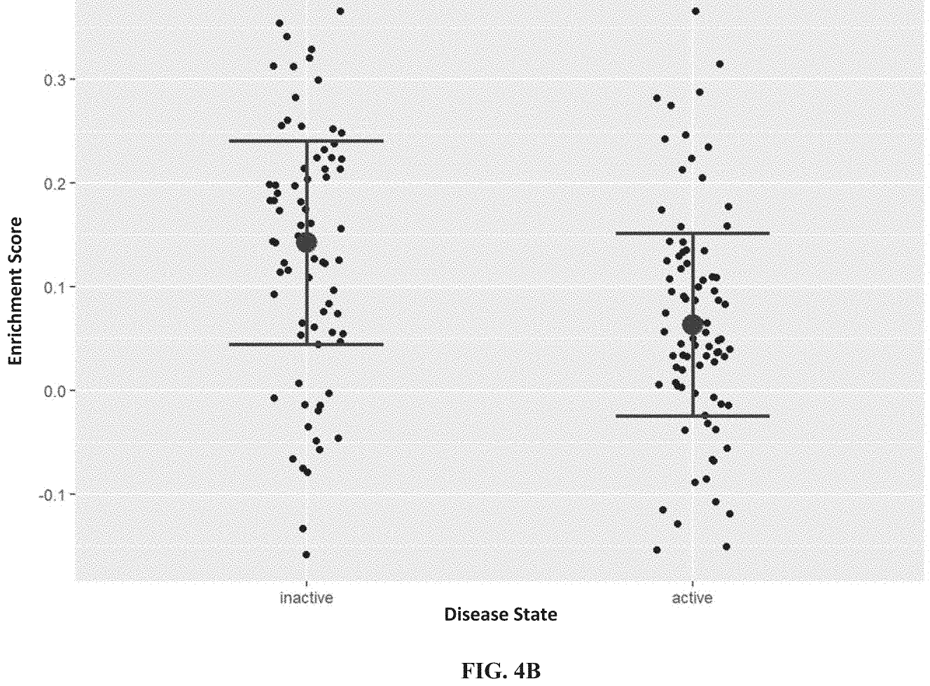

GSVA enrichment may be performed using the 25 cell-specific gene modules in WB from 156 records (82 active, 74 inactive), per Table 4 and FIG. 2E. Of the 25 cell-specific modules, 12 had enrichment scores with significant Spearman correlations to SLEDAI (p<0.05), and 14 had enrichment scores with significant differences between active and inactive state records by Welch's unequal variances t-test (p<0.05), per Table 2. Notably, each cell type produced at least one module with a significant correlation to SLEDAI in WB and at least one module with a significant difference in enrichment scores between active and inactive records, demonstrating a relationship between phenotypic activity in specific cellular subsets and overall phenotypic activity in WB. However, as the Spearman's rho values ranged from −0.40 to +0.36, no one module may have a substantial predictive value. Furthermore, the effect sizes as measured by Cohen's d when testing active versus inactive enrichment scores ranged from −0.85 to +0.79. The CD4 Floralwhite and Orangered4 modules, which had the largest positive and negative effect sizes, respectively, showed a high degree of overlap in the enrichment scores of active and inactive records, per FIGS. 4A and 4B, where error bars indicate mean±standard deviation. WB may be unable to fully separate active records from inactive records.

TABLE 2
Cell-specific modules by Spearman correlation
to SLEDAI and active vs. inactive state

| | Spearman | | | | |
|---|---|---|---|---|---|
| | correlation to | | Active vs. | | |
| | SLEDAI | | Inactive t-test | | |
| | | p | t sta- | p | Cohen's |
| | rho | value | tistic | value | d |
| CD4_Floralwhite | | 3.90E−06 | | 2.40E−06 | 0.788 |
| CD4_Turquoise | −0.044 | 0.587 | −0.93 | 0.352 | −0.149 |
| CD4_Orangered4 | −0.400 | 2.21E−07 | −5.29 | 4.35E−07 | −0.853 |
| CD14_Plum1 | 0.010 | 0.904 | −0.35 | 0.729 | −0.054 |
| CD14_Yellow | | 4.93E−06 | | 4.44E−06 | 0.761 |
| CD14_Greenyellow | −0.132 | 0.100 | −2.10 | 0.037 | −0.339 |
| CD14_Pink | −0.026 | 0.751 | 0.13 | 0.894 | 0.021 |
| CD14_Purple | −0.149 | 0.064 | −1.65 | 0.101 | −0.263 |
| CD14_Sienna3 | −0.368 | 2.27E−06 | −4.99 | 1.62E−06 | −0.799 |
| CD19_Darkolivegreen | 0.020 | 0.809 | −0.06 | 0.953 | −0.010 |
| CD19_Greenyellow | | 0.016 | | 0.012 | 0.403 |
| CD19_Steelblue | 0.016 | 0.838 | 0.55 | 0.580 | 0.089 |
| CD19_Turquoise | −0.069 | 0.393 | −0.84 | 0.403 | −0.132 |
| CD19_Violet | −0.087 | 0.282 | −1.48 | 0.141 | −0.236 |
| CD19_Brown | −0.050 | 0.537 | −1.04 | 0.301 | −0.164 |
| CD19_Green | −0.150 | 0.062 | −2.07 | 0.040 | −0.330 |
| CD19_Skyblue | −0.205 | 0.010 | −2.35 | 0.020 | −0.378 |
| CD33_Royalblue | | 8.99E−05 | | 1.03E−04 | 0.637 |
| CD33_Sienna3 | | 3.41E−06 | | 6.15E−06 | 0.753 |
| CD33_Violet | | 4.15E−05 | | 2.46E−05 | 0.696 |
| CD33_Darkmagenta | −0.216 | 6.74E−03 | −2.34 | 0.021 | −0.369 |
| LDG_A | −0.044 | 0.588 | −0.25 | 0.802 | −0.040 |
| LDG_B | | 5.71E−03 | | 0.019 | 0.377 |
| PC_Up | | 9.75E−04 | | 1.61E−03 | 0.508 |
| PC_Down | 0.022 | 0.781 | 0.80 | 0.426 | 0.129 |

Analysis of individual phenotypic activity associated peripheral cellular subset gene modules may not be sufficient to predict phenotypic activity in unrelated WB data sets, since no single module from any cell type may be able to separate active from inactive state records, per FIG. 2E. Although no single module had a sufficiently high predictive value, many cell-specific gene modules may be combined and optimized to predict phenotypes of active records. Moreover, the results emphasized the need for more advanced analysis to employ gene expression analysis to predict phenotypic activity.

### Performance and Accuracy

When training and testing sets are formed by holding out entire data sets, machine learning algorithms using raw gene expression data had an average classification accuracy of only 53 percent. However, converting this gene expression data to module enrichment improved classification accuracy to 71 percent. When training and testing sets are formed by mixing records from the three data sets, module enrichment remained at a 70 percent classification accuracy. However, classification accuracy using raw gene expression increased to a mean of 79 percent. The best overall performance came from the random forest classifier, which had a predictive accuracy of 84 percent.

The performance of each machine learning algorithm may be determined by evaluating 2 different forms of cross-validation. A random 10-fold cross-validation may randomly assign each record to one of 10 groups. A leave-one-study-out cross-validation may determine the effects of systematic technical differences among data sets on classification performance. For each pass of cross-validation, one fold or study may be held out as a test set, whereby the classifiers are trained on the remaining data. Accuracy may be assessed as the proportion of records correctly classified across all testing folds. Performance metrics such as sensitivity and specificity may be assessed after cross-validation by agglomerating class probabilities and assignments from each fold or study. Receiver Operating Characteristic (ROC) curves may be generated using the pROC R package.

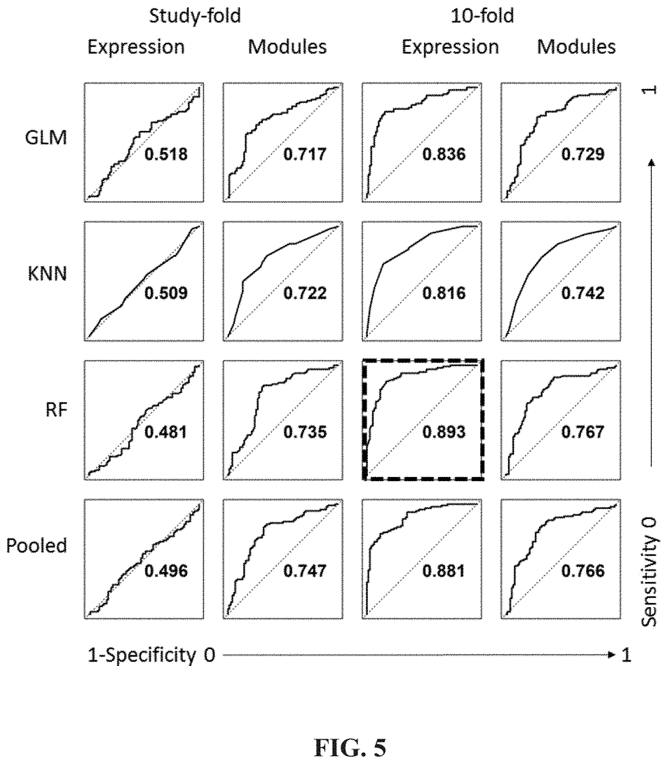

The performance of each classifier in each situation is shown in Table 3, and corresponding ROC curves are shown in FIG. 5, whereas the area under each ROC curve is displayed. In almost all cases, the random forest classifier outperformed the GLM and KNN classifiers, although the results may be not significantly different when assessed by testing for equality of proportions (p>0.05). Pooled predictions based on the class probabilities from the three classifiers may not improve overall performance.

TABLE 3
Cross-validation of gene expression and cell modules

| Study-fold Cross-Validation | | | 10-fold Cross-Validation | | |
|---|---|---|---|---|---|
| | Gene | | | Gene | |
| | Expres- | Cell | | Expres- | Cell |
| | sion | Modules | | sion | Modules |
| GLM | 0.56 | 0.68 | GLM | 0.80 | 0.72 |
| KNN | 0.48 | 0.68 | KNN | 0.75 | 0.7 |
| RF | 0.54 | 0.74 | RF | 0.84 | 0.72 |
| Pooled | 0.53 | 0.71 | Pooled | 0.78 | 0.73 |
| Mean | 0.53 | 0.70 | Mean | 0.79 | 0.72 |
| (SD) | (0.03) | (0.03) | (SD) | (0.04) | (0.01) |

When cross-validating by study, the use of expression values may achieve an accuracy of only 53 percent, per Table 3, which is consistent with the findings shown in FIGS. 2A-2D that gene expression values may provide less value towards classifying unfamiliar records. When the training records and test records are greatly heterogeneous, the classifiers learning patterns may be less helpful for classifying test records. Remarkably, the use of module enrichment scores improved accuracy to approximately 70 percent.

Per Table 3, the 10-fold cross-validation with raw gene expression values may result in better performance compared to the leave-one-study-out cross-validation. This increase in performance may be attributed to the presence of records from all plurality of first, second, and third records in both the training and test sets. In this case, the classifiers may learn patterns inherent to each set of records. In this circumstance, the random forest classifier may be the strongest performer with 84% accuracy (85% sensitivity, 83% specificity), whereby the ROC curve demonstrates an excellent tradeoff between recall and fall-out. The performance of module enrichment, however may not be substantially different between 10-fold cross-validation and leave-one-study-out cross-validation.

Overall, in a study-by-study approach (leave-one-study-out cross-validation), module enrichment may be more successful than raw gene expression. Importantly, when using the 10-fold cross-validation approach, raw gene expression may outperform module enrichment. Thus, phenotypic activity classification based on raw gene expression may be sensitive to technical variability, whereas classification based on module enrichment may cope better with variation among data sets.

The variable importance of Random forest provides insight into directors of the identification of phenotypic activity, random forest classifiers may be trained on all records from each of the plurality of records in order to identify the most important genes and modules as determined by mean decrease in the Gini impurity, a measure of misclassification error.

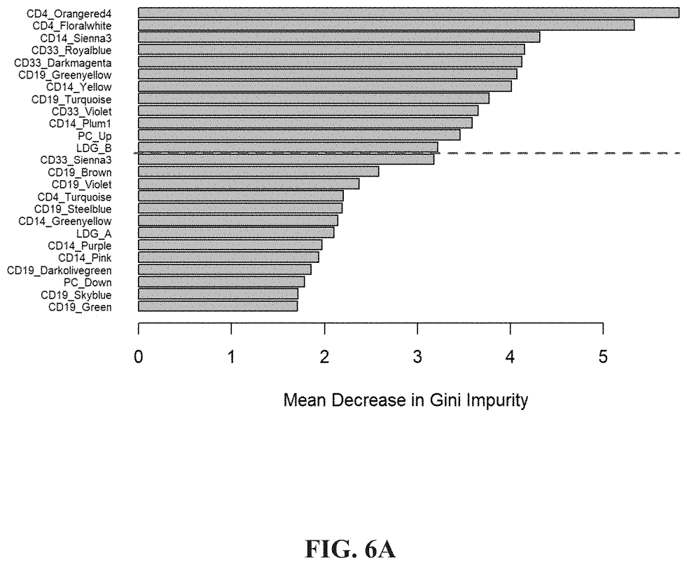

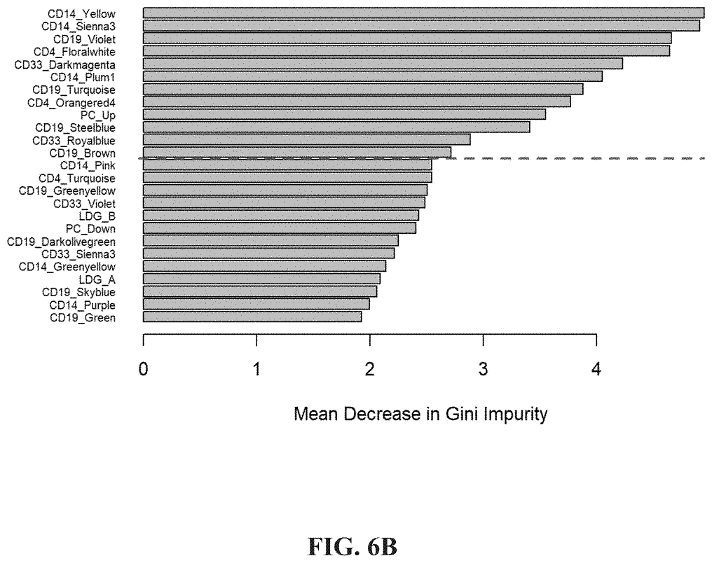

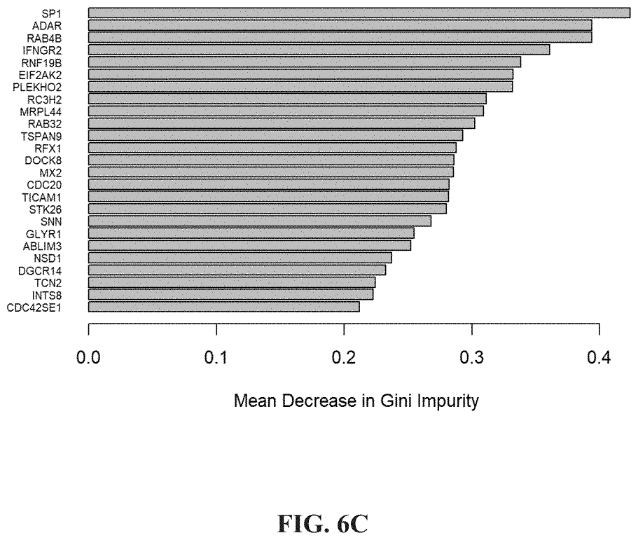

As shown in FIGS. 6A-6C, the most important genes and modules identified a wide array of cell types and biological functions. The most important genes encompass such diverse functions as interferon signaling, pattern recognition receptor signaling, and control of survival and proliferation, per FIG. 6C. Notably, the most influential modules may be skewed away from B cell-derived modules and towards T cell- and myeloid cell-derived modules, per FIG. 6A. As some of these modules had overlapping genes, the variable importance experiment may be repeated with modules that may be first scrubbed of any genes that appeared in more than one module before GSVA enrichment scoring. The relative variable importance scores of the de-duplicated modules correlated strongly with those of the original modules (Spearman's rho=0.73, p=5.18E-5), indicating that module behavior may be partly driven by the overlapping genes but strongly driven by unique genes, per FIG. 6A. Variable importance of top 25 individual genes. LDG: low-density granulocyte; PC: plasma cell.

CD4 Floralwhite and CD14 Yellow, two interferon-related modules which maintained high importance after deduplication, may be further analyzed to study the effect of unique genes on module importance. Gene lists may be tested for statistical overrepresentation of Gene Ontology biological process terms with FDR correction on pantherdb.org. CD4_Floralwhite did not show any significant enrichment, but CD14_Yellow, which had the highest importance after deduplication, may be highly enriched for genes with the “Immune Effector Process” designation (26/77 genes, FDR=9.38E-11 by Fisher's exact test). This suggests that CD14+ monocytes express unique genes that may play important roles in the initiation of phenotypic activity.

Several important findings on the topic of gene expression heterogeneity within and across data sets have been elucidated by this study. First, DE analysis of active vs inactive records may be insufficient for proper classification of phenotypic activity, as systematic differences between data sets render conventional bioinformatics techniques largely non-generalizable.

Further, WGCNA modules created from the cellular components of WB and correlated to SLEDAI phenotypic activity may improve classification of phenotypic activity in records. The use of cell-specific gene modules based on a priori knowledge about their relevance to disease fared slightly better than raw gene expression, as it generated informative enrichment patterns, and many of the modules maintained significant correlations with SLEDAI in WB. However, these enrichment scores failed to completely separate active records from inactive records by hierarchical clustering.

### Method Characterization

Conventional bioinformatics approaches do not satisfactorily identify one or more records having a specific phenotype. DE analysis of a plurality of first records, a plurality of second records, and a plurality of third records having an active disease state and a non-active disease state, per FIGS. 2A-2D displayed the major differences and heterogeneity. First, the 100 most significant DE genes by FDR in the plurality of first, second, and third records may be used to carry out hierarchical clustering of active and inactive disease state records, per FIGS. 2A-C. Active disease state records are clearly separated from inactive records, per FIG. 2B, but only partially separated from inactive records, per FIGS. 2A and 2C.

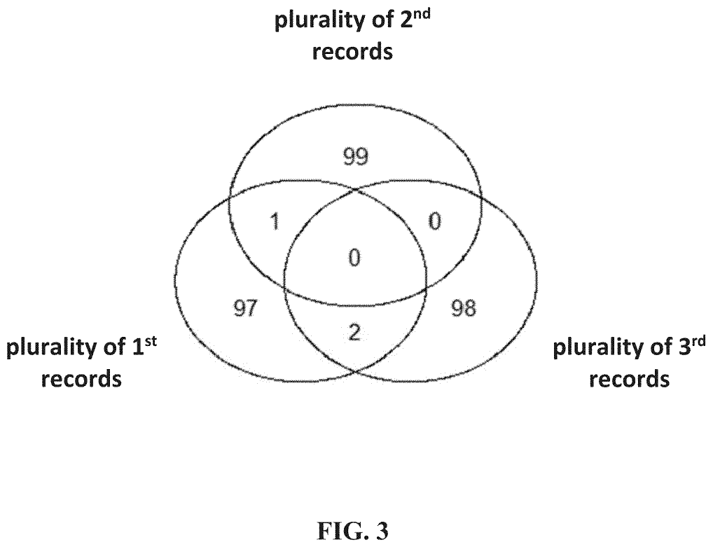

Out of 6,640 unique DE genes from the three pluralities of records, 5,170 genes are unique to one of the plurality of records, 1,234 are shared by two of the plurality of records, and 36 are shared by all three of the plurality of records. Per FIG. 3 there is minimal overlap of the 100 most significant genes by FDR in each of the pluralities of records. The only overlaps among the top 100 DE genes in each study by FDR are: TWY3 and EHBP1, shared between the plurality of first records and the plurality of third records; and LZIC, shared between the plurality of first records and plurality of second records. Furthermore, the fold change distributions of the 100 most significant DE genes in each of the pluralities of records varied considerably. In the plurality of first records, 94 of the 100 most significant genes are downregulated in active disease state records; in the plurality of second records, all of the top 100 genes are upregulated in active disease state records; and in the plurality of third records, the top 100 genes are more evenly distributed (41 up, 59 down). Per FIG. 3 orange bars denote active state records, wherein black bars denote inactive state records.

The plurality of first, second, and third records may represent different populations and may be collected on different microarray platforms per Table 4 below. The lack of commonality among the genes most descriptive of active state records and inactive state records in each of the pluralities of records casts doubt on whether active and inactive states from the different pluralities of records may be easily determined using conventional techniques.

TABLE 4
Accession of records by microarray platform, number of active
and inactive records, SLEDAI range, and SLEADAI mean

| | | N | N | | |
|---|---|---|---|---|---|
| | Microarray | Ac- | Inac- | SLEDAI | SLEDAI |
| Accession | Platform | tive | tive | Range | Mean (SD) |
| Plurality | GPL570 | 24 | 13 | 2-12 | 6.8 (2.7) |
| of First | (Affymetrix | | | | |
| Records | HG-U133+ 2.0) | | | | |
| Plurality | GPL13158 | 35 | 35 | 0-11 | 4.3 (3.5) |
| of Second | (Affymetrix | | | | |
| Records | HG-U133+ PM) | | | | |

Records from the pluralities of first, second, and third records may then be joined to evaluate whether unsupervised techniques may separate active state records from inactive state records. Hierarchical clustering on the 297 unique most significant DE genes by FDR showed considerable heterogeneity, and active records and inactive records did not consistently separate, per the heat map of the top 100 DE genes by FDR from each of the pluralities of records (combined total of 297 unique genes from the plurality of first, second, and third records) expressed in all records in FIG. 2D. As such, conventional techniques failed to identify active records, highlighting the need for more advanced algorithms.

### Digital Processing Device

In some embodiments, the platforms, systems, media, and methods described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, AmazonFire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing-based storage. In further embodiments, the storage and/or memory device is a combination of devices such as those disclosed herein.

In some embodiments, the digital processing device includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the digital processing device, such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

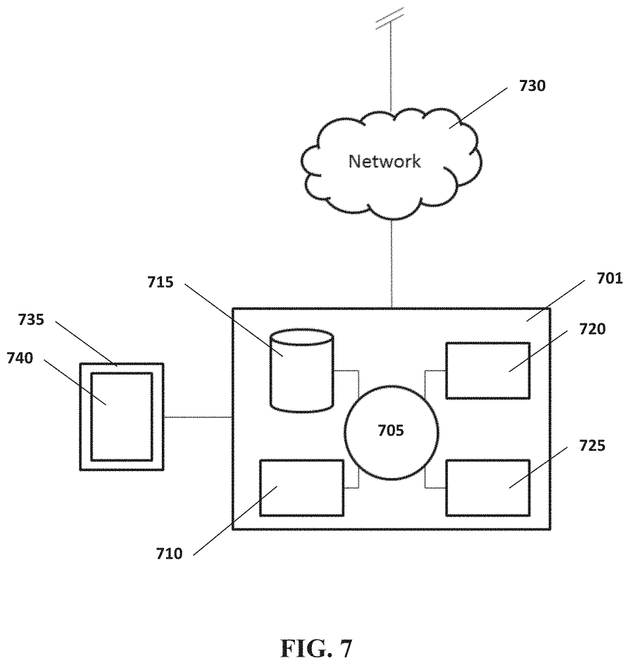

Referring to FIG. 7, in a particular embodiment, a digital processing device 701 is programmed or otherwise configured to identify one or more records having a specific phenotype. The device 701 is programmed or otherwise configured to identify one or more records having a specific phenotype. In this embodiment, the digital processing device 701 includes a central processing unit (CPU, also “processor” and “computer processor” herein) 705, which is optionally a single core, a multi core processor, or a plurality of processors for parallel processing. The digital processing device 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communication bus (solid lines), such as a motherboard. The storage unit 715 comprises a data storage unit (or data repository) for storing data. The digital processing device 701 is optionally operatively coupled to a computer network (“network”) 730 with the aid of the communication interface 720. The network 730, in various cases, is the internet, an internet, and/or extranet, or an intranet and/or extranet that is in communication with the internet. The network 730, in some cases, is a telecommunication and/or data network. The network 730 optionally includes one or more computer servers, which enable distributed computing, such as cloud computing. The network 730, in some cases, with the aid of the device 701, implements a peer-to-peer network, which enables devices coupled to the device 701 to behave as a client or a server.

Continuing to refer to FIG. 7, the CPU 705 is configured to execute a sequence of machine-readable instructions, embodied in a program, application, and/or software. The instructions are optionally stored in a memory location, such as the memory 710. The instructions are directed to the CPU 705, which subsequently program or otherwise configure the CPU 705 to implement methods of the present disclosure. Examples of operations performed by the CPU 705 include fetch, decode, execute, and write back. The CPU 705 is, in some cases, part of a circuit, such as an integrated circuit. One or more other components of the device 701 are optionally included in the circuit. In some cases, the circuit is an application specific integrated circuit (ASIC) or a field programmable gate array (FPGA).

Continuing to refer to FIG. 7, the storage unit 715 optionally stores files, such as drivers, libraries and saved programs. The storage unit 715 optionally stores user data, e.g., user preferences and user programs. The digital processing device 701, in some cases, includes one or more additional data storage units that are external, such as located on a remote server that is in communication through an intranet or the internet.

Continuing to refer to FIG. 7, the digital processing device 701 optionally communicates with one or more remote computer systems through the network 730. For instance, the device 701 optionally communicates with a remote computer system of a user. Examples of remote computer systems include personal computers (e.g., portable PC), slate or tablet PCs (e.g., Apple® iPad, Samsung® Galaxy Tab, etc.), smartphones (e.g., Apple® iPhone, Android-enabled device, Blackberry®, etc.), or personal digital assistants.

Methods as described herein are optionally implemented by way of machine (e.g., computer processor) executable code stored on an electronic storage location of the digital processing device 701, such as, for example, on the memory 710 or electronic storage unit 715. The machine executable or machine readable code is optionally provided in the form of software. During use, the code is executed by the processor 705. In some cases, the code is retrieved from the storage unit 715 and stored on the memory 710 for ready access by the processor 705. In some situations, the electronic storage unit 715 is precluded, and machine-executable instructions are stored on the memory 710.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

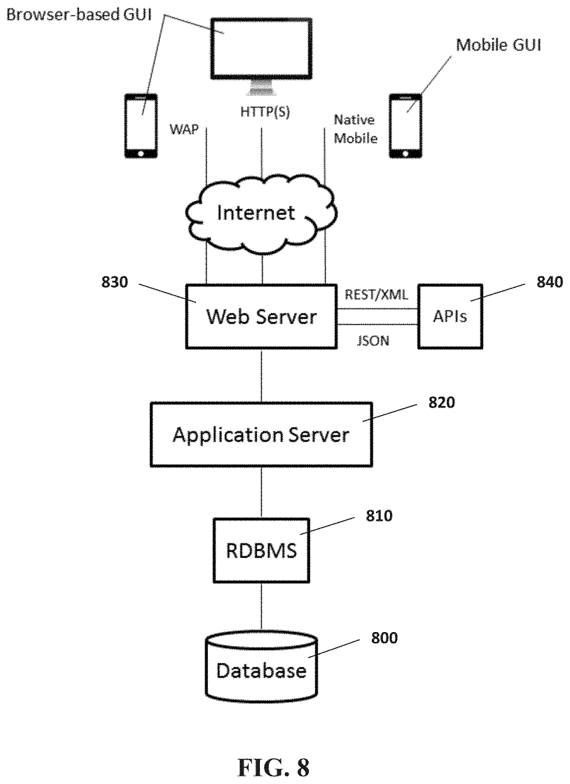

Referring to FIG. 8, in a particular embodiment, an application provision system comprises one or more databases 800 accessed by a relational database management system (RDBMS) 810. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 820 (such as Java servers, .NET servers, PHP servers, and the like) and one or more web servers 830 (such as Apache, IIS, GWS and the like). The web server(s) optionally expose one or more web services via app application programming interfaces (APIs) 840. Via a network, such as the internet, the system provides browser-based and/or mobile native user interfaces.

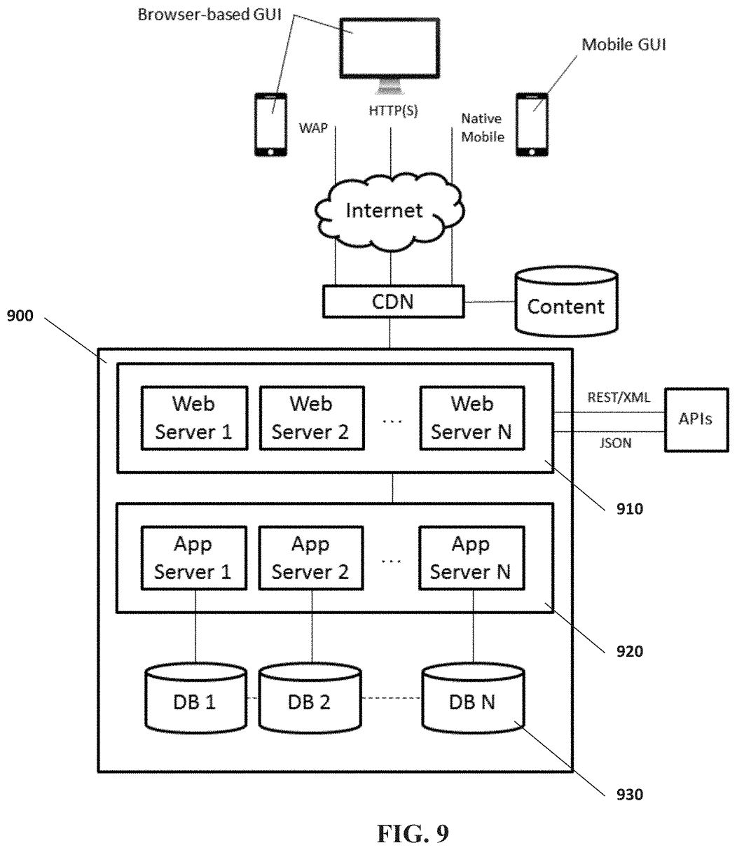

Referring to FIG. 9, in a particular embodiment, an application provision system alternatively has a distributed, cloud-based architecture 900 and comprises elastically load balanced, auto-scaling web server resources 910 and application server resources 920 as well synchronously replicated databases 930.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-in

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer °, Mozilla® Firefox, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for identifying one or more records having a specific phenotype. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### Interferon Profiling of Lupus Conditions

A role for interferon (IFN) in SLE pathogenesis may be inferred from the prominent IFN gene signature (IGS), but the major IFN species and its relationship to SLE disease activity may be unknown. Bioinformatic approaches may employ gene signatures specific for individual IFN species to interrogate SLE microarray datasets toward ascertaining the roles of individual IFN species.

A role for interferon (IFN) in SLE pathogenesis may be inferred from the prominent IFN gene signature (IGS), but the major IFN species and its relationship to SLE disease activity may be unknown. A bioinformatic approach employing gene signatures specific for individual IFN species to interrogate SLE microarray datasets may demonstrate a putative role for numerous IFN species, with prominent expression of IFNB1 and IFNW induced genes, and concordance between IFN signatures in MS patients treated with IFNB1 and SLE-affected skin and synovium compared to SLE nephritis, suggesting that IFN signaling is less prominent in SLE renal disease. SLE patients with inactive disease have readily detectable IGS, and the IGS changed synchronously with a monocyte signature but not disease activity, and was significantly related to monocyte transcripts. Monocyte over-expression of three times as many IGS transcripts as T cells and B cells and IGS retention in monocytes, but not T cells and B cells from inactive SLE patients contribute to the lack of correlation between the IGS and SLE disease activity.

A role for interferon (IFN) in the pathogenesis of systemic lupus erythematosus (SLE) has been proposed since early experiments showed elevated IFN activity in SLE patients and the advent of gene expression profiling demonstrated a robust IFN gene signature (IGS) in SLE patient peripheral blood, purified B cells, T cells, monocytes, and affected organs. Various IFN responsive genes have been used to define the IGS but little is understood regarding the specific species of IFN underlying the signature. Notably, there remains a lack of consensus concerning the association of the IGS with SLE disease activity. Although some disease metrics have been associated with the IGS in small studies, longitudinal studies may not show correlation between the IGS and disease activity.

Anecdotal accounts of patients developing SLE-like symptoms after treatment with IFNs have been reported, suggesting that IFN might play a role in the induction of SLE. Moreover, standard of care (SOC) drugs used to treat lupus may eliminate the IGS. Two anti-IFNA antibodies have been used to treat SLE in Phase II clinical trials but with only modest effects. In contrast, a trial using the antibody anifrolumab, which blocks binding of all type I IFNs to the shared IFN receptor, provided clinically meaningful benefit in subjects with SLE and with high IGS scores. These trials raise the important question of whether IFNA (IFN-alpha or IFN-α) is the predominant IFN acting in SLE.

An IGS may be induced by type I or type II IFNs. The human type I IFN locus comprises thirteen IFNA genes (A1, A2, A4, A5, A6, A7, A8, A10, A13, A14, A16, A17, and A21), IFNB1 (IFN-beta1 or IFN-β1), IFNW1 (IFN-omega1 or IFN-ω1), and IFNE (IFN-epsilon or IFN-8). Despite a similarity in structure and common receptor, these IFNs may induce different downstream signaling events, although mRNA signatures to distinguish the action of a specific subtype of type I IFN have not been developed or employed to delineate the actions of specific Type 1 IFNs. The type II IFN, IFNG (IFN-gamma or IFN-γ), also induces an IGS through its distinct IFNG receptor and has been shown to be important for pathogenesis in lupus mouse models. The role of IFNG in the pathogenesis of human lupus has been inferred largely through in vitro experiments.

Deconvolution of the IGS in SLE may be performed by creating three modules of IFN genes (M1.2, M3.4, M5.12) from SLE microarray datasets clustered using a K-means algorithm on the basis of their expression. Some correlation between module 5.12 with SLE flares may be noted, and characterization of the module using the IFN database, the Interferome, may be done in an attempt to classify the species of IFN. However, the Interferome may not necessarily reflect the downstream microarray signature present in human cells and tissues.

In order to delineate the specific types of IFNs present in SLE and the potential role of specific IFNs in SLE disease processes, systems and methods provided herein may employ a systems-level approach by using multiple, publicly available gene expression datasets from SLE patients, and probing them using reference datasets of the downstream IGS induced in vitro in human peripheral blood mononuclear cells (PBMC) or in vivo in whole blood (WB) by administration of specific IFNs to patients. This approach may allow the determination of the relative contributions of different types of IFN in SLE affected cells and tissues as well as a better understanding of the IGS and its relationship to SLE disease processes.

The present disclosure provides systems and methods to interrogate the IGS in SLE microarray datasets using reference datasets. The use of microarray data from unrelated yet relevant datasets as a tool for microarray dataset interrogation is an important advance, since it does not rely on prior characterization or knowledge of any genes, and also focuses the analysis on gene changes that have been shown to be operative in human samples. Using systems and methods described herein, strong enrichment may be demonstrated for IFNB1 in the SLE skin and synovium, and importantly a strong similarity may be shown between signatures in patients treated chronically with IFNB1 and the SLE WB signature. Moreover, the IGS may be related to monocytes in the analyzed samples.

Z score calculations and GSVA enrichment scores may demonstrate the likely role of IFNB1 in SLE pathogenesis, and suggest that targeting these IFNs in lupus skin and synovium may be more beneficial than blocking IFN in SLE patients with proliferative LN. Effect size values for GSVA enrichment scores and Z scores for IFNs are lower in LN tissue, and about 20% of LN patients may lack a type I IGS. The finding that the kidneys differ from skin and synovium may be unexpected and may not be anticipated from the blood analysis, thereby demonstrating the important contributions of tissue samples to results disclosed herein. Single-cell analysis of hematopoietic cells derived from the kidneys of LN patients demonstrates a low IGS in cells from most patients. These results together with our data may suggest that the IFN signaling pathway may not be as prominent in this tissue compared to skin and synovium. Noting that both skin and synovium are rich in fibroblasts, an important IFNB1 producing cell type, that constitutive IFNB1 production may provide a background of IFN in these tissues whereas the normal kidney has relatively few fibroblasts.

The greater association between the MS-IFNB1 signature and the SLE IGS signature may be of particular note. The much higher Z scores calculated using the MS-IFNB1 signature for all WB, PBMC, and SLE affected tissues in comparison to the calculated GSVA enrichment scores may be related to the increased overlap of decreased transcripts between the MS-IFNB1 signature and the signature in SLE patients. Long-term exposure to IFNB1 in MS patients may lead to a decrease in transcripts such as CD1C, CD160, IGF1R, and TNFRSF9 (4-1BB) that are also seen in SLE patients. All of these molecules participate in cellular activation, and inhibition of them after long-term exposure to IFNB1 may suggest a shared down-regulatory mechanism between MS patients treated with IFNB1 and SLE patients. Little evidence is shown for enrichment of the non-canonical IFNB1 signaling pathway in SLE affected tissues, however, this conclusion may be tempered by the use of a murine signature derived from IFNAR2 deficient peritoneal exudate cells as a comparator.

Although results show strong enrichment of IFNB1 in SLE, they may not preclude a role for the IFNAs. Indeed, IFNB1 itself has been shown to induce the expression of IFNAs. The two-step model of type I IFN induction by viruses, TLR, or other cytosolic pattern recognition receptors may establish that the activation of the constitutively expressed IRF3 in the cytoplasm leads to the initial induction of only IFNB1. The induced IFNB1 acts on the IFNA/B receptor to induce IRF7 expression by activating ISGF3 in the cytoplasm leading to the induction of IFNAs. IFNW1 is among the most induced genes in humans, along with IFNA2 and IFNB1, after pDC treatment with TLR7 agonists.

The IFNG signature has significant effect size and Z scores for all SLE tissues and most peripheral datasets, albeit lower than the three type I signatures. The induction of type I IFNs in response to virus initiates a cascade of events leading to the recruitment and/or activation of CD8 T cells and natural killer (NK) cells. While IFNG is induced in CD8 T cells, NK cells constitutively express IFNG transcripts, and NK cells are not easily discernible from CD8 T cells by microarray expression. In lupus mouse models, IFNG appears to play a more prominent role than in humans, and a hypothesis is proposed that the presence of IFNG may represent a late stage response to the inappropriate induction of type I IFNs in response to sterile inflammatory stimuli.

Using systems and methods disclosed herein, it may be shown that inactive SLE patients have a readily detectable IGS and that some SLE patients over time may change their IGS status. In two longitudinal datasets assessing SLE patients treated with standard of care (SOC) medications (GSE88885, GSE88886), the gain or loss of the IGS is demonstrated in about 30% of subjects. This change in status in the absence of intense immunotherapy may suggest that the IGS is not stable during the disease process in one third of SLE patients. The results disclosed herein, involving more than 2000 patients, may suggest that there is not a relationship between SLEDAI and the IGS. Additionally, about 30% of the 119 SLE patients on standard of care (SOC) treatment significantly changed their IGS over a one-year period. Notably, no predictable relationship may be measured between the SLEDAI and IGS. In ten SLE LN patients (GSE72747), the IGS did not change synchronously with the SLEDAI, and the change in IGS may be shown to be associated with a change in monocytes.

Because of the high degree of heterogeneity in both SLE patients and in microarray dataset platforms, processing and controls, a meta-analysis approach can be performed in order to understand and interpret the relationship between gene expression signatures to each other and disease activity. Linear regression analysis of the SLEDAI and GSVA scores for cell types, cellular processes, or IGS for seven SLE datasets show the strongest relationship to the SLEDAI is expression of genes regulating the cell cycle. This may be reassuring, as this cell cycle signature is taken from a WGCNA plasma cell module in SLE CD19 B cells correlated to SLEDAI, and plasma cells have been shown to correlate with SLEDAI. A plasma cell signature comprised of immunoglobulin (Ig) genes as well as other hallmark genes of plasma cells is also correlated to SLEDAI, although this full signature may not be detected in datasets on the Illumina platform because of the absence of Ig genes and may be underestimated on microarray chips in general because of their limited number of Ig genes. The IFN core, IFNW1, and IFNB1 signatures have low positive correlations with SLEDAI, and as was the case for the cell cycle and plasma cell signatures, have low predictive value for the SLEDAI.

A predictive relationship across ten SLE WB and PBMC datasets (2152 patients) is determined for all the IGS and monocyte cell surface transcripts with a range of r² predictive values of 0.29-0.58. This may suggest that the IGS is most related to the increased presence of monocytes expressing the IGS. Three times as many transcripts from the IFN core signature were enriched in monocytes relative to T cells and B cells. However, whereas some members of the IGS in SLE were highly overexpressed in SLE monocytes (e.g., EIF2AK2, OASL, OAS2, OAS3, PLSCR1, and CXCL10), some of the most overexpressed transcripts when SLE patients were compared to HC, including IFI27, IFI44L, IFIH1, IFIT3, OASL, RSAD2, SPATS2L and USP18, are not over-expressed in SLE monocytes compared with SLE T cell and B cells. Support for monocytes having a greater intensity IGS may be shown in experiments in which the log signal ratios of a 20-gene IGS are compared between purified T cells, B cells, and monocytes in SLE patients.

In addition to monocytes from active SLE patients expressing a greater intensity for ⅔ of the IFN core transcripts, another contributing factor for the strong relationship of monocytes to the IGS may be found by studying the IGS in purified T cells, B cells, and monocytes from subjects with inactive SLE. The T cell and B cell WGCNA-derived IFN modules may correlate significantly to SLEDAI, whereas the CD14 monocyte IFN module may not. The presence of an IGS in CD14 monocytes, but not in CD4 T and CD19 B cells from inactive patients, may support that monocytes are maintaining the IGS in inactive SLE patients. One explanation for this may be the increased STAT1 transcripts found in inactive SLE WB, PBMC, and monocyte datasets, but not the inactive SLE CD4 T or CD19 B cells. A prolonged IGS in monocytes in the absence IFN may also explain why some patients with IGS signatures have no IFNA detected using an ultrasensitive ELISA.

Another possible explanation for how monocytes may maintain an enhanced IGS derives from experiments treating human monocytes with a combination of TNF and IFN on a background of TLR signaling. IFN treatment in this context leads to epigenetic changes allowing for a much greater IGS than when cells are stimulated with IFN alone. Thus, the presence of inflammatory cytokines such as TNF, along with nucleic acid-containing immune complexes capable of signaling through TLRs, may account for the prolonged IGS seen in monocytes even when disease activity is low. Further work to elucidate the specific relationship between WB signatures and matching signatures from SLE affected tissues may improve understanding of this prominent signature and its association with an increased monocyte gene signature.

IFNB1 presents an intriguing target for SLE therapy because of the predominance of its signature in SLE affected tissues, its unique signaling properties and cellular expression, and its potential role in B cell development and tolerance. However, as shown by the results herein, the IGS may not correlate with the SLEDAI disease measurement, and a prolonged IGS in monocytes may make interpretation of the IGS as a measure of disease activity or the immediate presence of IFN challenging. The potential benefit of targeting IFNB1 may be considered within the practical limitations of disease measurement indices used in SLE clinical trials. It may be of critical importance that disease measurements truly reflect a change in the tissue manifestations of SLE.

In one aspect, the present disclosure provides a method for identifying a lupus condition of a subject, comprising: (a) assaying a biological sample of the subject to generate a dataset comprising gene expression data; (b) processing the dataset at each of a plurality of genes to determine quantitative measures of each of the plurality of genes, wherein the plurality of genes comprises genes induced by a plurality of interferons, thereby producing an interferon signature of the biological sample of the subject; (c) comparing the interferon signature with one or more reference interferon signatures, wherein the comparing comprises, for at least one of the plurality of genes, comparing the quantitative measure of the gene of the interferon signature with corresponding quantitative measures of the gene of the one or more reference interferon signatures; and (d) based at least in part on the comparison in (c), identifying the lupus condition of the subject.

In some embodiments, the lupus condition is selected from the group consisting of: systemic lupus erythematosus (SLE), discoid lupus erythematosus (DLE), and lupus nephritis (LN). In some embodiments, the biological sample is selected from the group consisting of: a whole blood (WB) sample, a PBMC sample, a tissue sample, and a purified cell sample. In some embodiments, the tissue sample is selected from the group consisting of: skin tissue, synovium tissue, and kidney tissue. In some embodiments, the kidney tissue is selected from the group consisting of: glomerulus (Glom) and tubulointerstitium (TI). In some embodiments, the purified sample is selected from the group consisting of: purified CD4⁺ T cells, purified CD19⁺ B cells, and purified CD14⁺ monocytes.

In some embodiments, the method further comprises purifying a whole blood sample of the subject to obtain the purified cell sample. In some embodiments, assaying the biological sample comprises (i) using a microarray to generate the dataset comprising the gene expression data, (ii) sequencing the biological sample to generate the dataset comprising the gene expression data, or (iii) performing quantitative polymerase chain reaction (qPCR) of the biological sample to generate the dataset comprising the gene expression data.

In some embodiments, the plurality of interferons comprises Type I interferons and/or Type II interferons. In some embodiments, the Type I interferons and/or Type II interferons are selected from the group consisting of IFNA2, IFNB1, IFNW1, and IFNG. In some embodiments, the plurality of genes comprises one or more genes induced by in vitro stimulation of PBMC by the plurality of interferons. In some embodiments, the one or more genes induced by in vitro stimulation of PBMC are selected from the genes listed in Table 13. In some embodiments, the one or more genes induced by in vitro stimulation of PBMC are selected from the genes listed in Table 14. In some embodiments, the one or more genes induced by in vitro stimulation of PBMC are selected from the genes listed in Table 15. In some embodiments, the one or more genes induced by in vitro stimulation of PBMC are selected from the genes listed in Table 16. In some embodiments, the plurality of genes comprises one or more genes induced by in vitro stimulation of PBMC by IL12 treatment or TNF treatment. In some embodiments, the one or more genes induced by in vitro stimulation of PBMC are selected from the genes listed in Table 17. In some embodiments, the one or more genes induced by in vitro stimulation of PBMC are selected from the genes listed in Table 18. In some embodiments, the plurality of genes comprises one or more genes induced in vivo in IFNA2-treated HepC patients and/or IFNB1-treated MS patients. In some embodiments, the one or more genes induced in vivo in IFNA2-treated HepC patients and/or IFNB1-treated MS patients are selected from the genes listed in Table 25.

In some embodiments, the quantitative measures of each of the plurality of genes comprise enrichment scores of each of the plurality of genes. In some embodiments, the enrichment scores of each of the plurality of genes comprise gene set variation analysis (GSVA) enrichment scores of each of the plurality of genes.

In some embodiments, (c) further comprises, for the at least one of the plurality of genes, determining a difference between the quantitative measure of the gene of the interferon signature with the corresponding quantitative measures of the gene of the one or more reference interferon signatures. In some embodiments, (d) further comprises identifying the lupus condition of the subject when the difference satisfies a pre-determined criterion. In some embodiments, (c) further comprises, for the at least one of the plurality of genes, determining a Z-score of the quantitative measure of the gene of the interferon signature relative to the corresponding quantitative measures of the gene of the one or more reference interferon signatures. In some embodiments, (d) further comprises identifying the lupus condition of the subject when the Z-score satisfies a pre-determined criterion. In some embodiments, (d) further comprises identifying the presence of the lupus condition of the subject when the Z-score is at least 2, and identifying the absence of the lupus condition of the subject when the Z-score is less than 2.

In some embodiments, the method further comprises identifying the lupus condition of the subject at a sensitivity of at least about 70%. In some embodiments, the method further comprises identifying the lupus condition of the subject at a sensitivity of at least about 80%. In some embodiments, the method further comprises identifying the lupus condition of the subject at a sensitivity of at least about 90%.

In some embodiments, the method further comprises identifying the lupus condition of the subject at a specificity of at least about 70%. In some embodiments, the method further comprises identifying the lupus condition of the subject at a specificity of at least about 80%. In some embodiments, the method further comprises identifying the lupus condition of the subject at a specificity of at least about 90%.

In some embodiments, the method further comprises identifying the lupus condition of the subject at a positive predictive value (PPV) of at least about 70%. In some embodiments, the method further comprises identifying the lupus condition of the subject at a positive predictive value (PPV) of at least about 80%. In some embodiments, the method further comprises identifying the lupus condition of the subject at a positive predictive value (PPV) of at least about 90%.

In some embodiments, the method further comprises identifying the lupus condition of the subject at a negative predictive value (NPV) of at least about 70%. In some embodiments, the method further comprises identifying the lupus condition of the subject at a negative predictive value (NPV) of at least about 80%. In some embodiments, the method further comprises identifying the lupus condition of the subject at a negative predictive value (NPV) of at least about 90%.

In some embodiments, the method further comprises identifying the lupus condition of the subject with an Area Under Curve (AUC) of at least about 0.70. In some embodiments, the method further comprises identifying the lupus condition of the subject with an Area Under Curve (AUC) of at least about 0.80. In some embodiments, the method further comprises identifying the lupus condition of the subject with an Area Under Curve (AUC) of at least about 0.90.

In some embodiments, the method further comprises determining or predicting an active or inactive state of the identified lupus condition of the subject. In some embodiments, (d) further comprises identifying the lupus condition of the subject based at least in part on a SLEDAI score of the subject. In some embodiments, the subject is asymptomatic for one or more lupus conditions selected from the group consisting of: systemic lupus erythematosus (SLE), discoid lupus erythematosus (DLE), and lupus nephritis (LN).

In some embodiments, the method further comprises applying a trained algorithm to the interferon signature to identify the lupus condition of the subject. In some embodiments, the trained algorithm is trained using a first set of independent training samples associated with a presence of the lupus condition and a second set of independent training samples associated with an absence of the lupus condition. In some embodiments, the method further comprises using the trained algorithm to process a set of clinical health data of the subject to identify the lupus condition. In some embodiments, the trained algorithm comprises a supervised machine learning algorithm. In some embodiments, the supervised machine learning algorithm comprises a deep learning algorithm, a support vector machine (SVM), a neural network, or a Random Forest.

In some embodiments, (a) comprises (i) subjecting the biological sample to conditions that are sufficient to isolate, enrich, or extract a plurality of nucleic acid molecules; and (ii) analyzing the plurality of nucleic acid molecules to generate the dataset comprising the gene expression data. In some embodiments, the method further comprises using probes configured to selectively enrich the plurality of nucleic acid molecules corresponding to a panel of one or more genomic loci. In some embodiments, the probes are nucleic acid primers. In some embodiments, the probes have sequence complementarity with nucleic acid sequences of the panel of the one or more genomic loci. In some embodiments, the panel of the one or more genomic loci comprises genomic loci corresponding to the plurality of genes. In some embodiments, the panel of the one or more genomic loci comprises at least 5 distinct genomic loci. In some embodiments, the panel of the one or more genomic loci comprises at least 10 distinct genomic loci.

In some embodiments, the method further comprises (e) assaying a second biological sample of the subject to generate a second dataset comprising gene expression data; (f) processing the second dataset at each of the plurality of genes to determine second quantitative measures of each of the plurality of genes, thereby producing a second interferon signature of the second biological sample of the subject; (g) comparing the second interferon signature with one or more reference interferon signatures, wherein the comparing comprises, for at least one of the plurality of genes, comparing the quantitative measure of the gene of the second interferon signature with corresponding quantitative measures of the gene of the one or more reference interferon signatures; and (h) based at least in part on the comparison in (g), identifying the lupus condition of the subject.

In some embodiments, the biological sample and the second biological sample comprise two different sample types selected from the group consisting of: a whole blood (WB) sample, a PBMC sample, a skin tissue sample, a synovium tissue sample, a kidney tissue sample comprising glomerulus (Glom), a kidney tissue sample comprising tubulointerstitium (TI), a purified CD4⁺ T cell sample, a purified CD19⁺ B cell sample, and a purified CD14⁺ monocyte sample.

In some embodiments, the method further comprises determining a likelihood of the identification of the lupus condition of the subject. In some embodiments, the method further comprises providing a therapeutic intervention for the lupus condition of the subject.

In some embodiments, the method further comprises monitoring the lupus condition of the subject, wherein the monitoring comprises assessing the lupus condition of the subject at a plurality of time points, wherein the assessing is based at least on the lupus condition identified in (d) at each of the plurality of time points. In some embodiments, a difference in the assessment of the lupus condition of the subject among the plurality of time points is indicative of one or more clinical indications selected from the group consisting of: (i) a diagnosis of the lupus condition of the subject, (ii) a prognosis of the lupus condition of the subject, and (iii) an efficacy or non-efficacy of a course of treatment for treating the lupus condition of the subject.

In some embodiments, the one or more reference interferon signatures are generated by: assaying a biological sample of one or more patients with dermatomyositis to generate a reference dataset comprising gene expression data; and processing the reference dataset at each of the plurality of genes to determine quantitative measures of each of the plurality of genes.

In another aspect, the present disclosure provides a computer system for identifying a lupus condition of a subject, comprising: a database that is configured to store a dataset comprising gene expression data, wherein the gene expression data is obtained by assaying a biological sample of the subject; and one or more computer processors operatively coupled to the database, wherein the one or more computer processors are individually or collectively programmed to: (i) process the dataset at each of a plurality of genes to determine quantitative measures of each of the plurality of genes, wherein the plurality of genes comprises genes induced by a plurality of interferons, thereby producing an interferon signature of the biological sample of the subject; (ii) compare the interferon signature with one or more reference interferon signatures, wherein the comparing comprises, for at least one of the plurality of genes, comparing the quantitative measure of the gene of the interferon signature with corresponding quantitative measures of the gene of the one or more reference interferon signatures; and (iii) based at least in part on the comparison in (ii), identify the lupus condition of the subject.

In some embodiments, the computer system further comprises an electronic display operatively coupled to the one or more computer processors, wherein the electronic display comprises a graphical user interface that is configured to display the report.

In another aspect, the present disclosure provides a non-transitory computer readable medium comprising machine-executable code that, upon execution by one or more computer processors, implements a method for identifying a lupus condition of a subject, the method comprising: (a) assaying a biological sample of the subject to generate a dataset comprising gene expression data; (b) processing the dataset at each of a plurality of genes to determine quantitative measures of each of the plurality of genes, wherein the plurality of genes comprises genes induced by a plurality of interferons, thereby producing an interferon signature of the biological sample of the subject; (c) comparing the interferon signature with one or more reference interferon signatures, wherein the comparing comprises, for at least one of the plurality of genes, comparing the quantitative measure of the gene of the interferon signature with corresponding quantitative measures of the gene of the one or more reference interferon signatures; and (d) based at least in part on the comparison in (c), identifying the lupus condition of the subject.

In another aspect, the present disclosure provides a method for identifying a sepsis condition of a subject, comprising: (a) assaying a biological sample of the subject to generate a dataset comprising gene expression data; (b) processing the dataset at each of a plurality of genes to determine quantitative measures of each of the plurality of genes, wherein the plurality of genes comprises genes induced by TNF, thereby producing a TNF signature of the biological sample of the subject; (c) comparing the TNF signature with one or more reference TNF signatures, wherein the comparing comprises, for at least one of the plurality of genes, comparing the quantitative measure of the gene of the TNF signature with corresponding quantitative measures of the gene of the one or more reference TNF signatures; and (d) based at least in part on the comparison in (c), identifying the sepsis condition of the subject.

### Certain Terms

As used herein, the singular forms “a,” “an,” and “the” include plural references unless the context clearly dictates otherwise. Any reference to “or” herein is intended to encompass “and/or” unless otherwise stated.

As used herein, the term “subject” refers to an entity or a medium that has testable or detectable genetic information. A subject can be a person, individual, or patient. A subject can be a vertebrate, such as, for example, a mammal. Non-limiting examples of mammals include humans, simians, farm animals, sport animals, rodents, and pets. The subject may be displaying a symptom(s) indicative of a health or physiological state or condition of the subject, such as a disease or disorder of the subject. As an alternative, the subject can be asymptomatic with respect to such health or physiological state or condition.

As used herein, the term “sample,” generally refers to a biological sample obtained from or derived from one or more subjects. Biological samples may be processed or fractionated before further analysis. Biological samples may include a whole blood (WB) sample, a PBMC sample, a tissue sample, a purified cell sample, or derivatives thereof. For example, a tissue sample may comprise skin tissue, synovium tissue, kidney tissue (e.g., glomerulus (Glom) or tubulointerstitium (TI)), or derivatives thereof. For example, a purified cell sample may comprise purified CD4⁺ T cells, purified CD19⁺ B cells, purified CD14⁺ monocytes, or derivatives thereof. In some embodiments, a whole blood sample may be purified to obtain the purified cell sample. The term “derived from” used herein refers to an origin or source, and may include naturally occurring, recombinant, unpurified or purified molecules.

To obtain a blood sample, various techniques may be used, e.g., a syringe or other vacuum suction device. A blood sample can be optionally pre-treated or processed prior to use. A sample, such as a blood sample, may be analyzed under any of the methods and systems herein within 4 weeks, 2 weeks, 1 week, 6 days, 5 days, 4 days, 3 days, 2 days, 1 day, 12 hr, 6 hr, 3 hr, 2 hr, or 1 hr from the time the sample is obtained, or longer if frozen. When obtaining a sample from a subject (e.g., blood sample), the amount can vary depending upon subject size and the condition being screened. In some embodiments, at least 10 mL, 5 mL, 1 mL, 0.5 mL, 250, 200, 150, 100, 50, 40, 30, 20, 10, 9, 8, 7, 6, 5, 4, 3, 2, or 1 μL of a sample is obtained. In some embodiments, 1-50, 2-40, 3-30, or 4-20 μL of sample is obtained. In some embodiments, more than 5, 10, 15, 20, 25, 30, 35, 40, 45, 50, 55, 60, 65, 70, 75, 80, 85, 90, 95 or 100 μL of a sample is obtained.

As used herein the term “diagnose” or “diagnosis” of a status or outcome includes predicting or diagnosing the status or outcome, determining predisposition to a status or outcome, monitoring treatment of patient, diagnosing a therapeutic response of a patient, and prognosis of status or outcome, progression, and response to particular treatment.

The sample may be taken before and/or after treatment of a subject with a disease or disorder. Samples may be obtained from a subject during a treatment or a treatment regime. Multiple samples may be obtained from a subject to monitor the effects of the treatment over time. The sample may be taken from a subject known or suspected of having a disease or disorder for which a definitive positive or negative diagnosis is not available via clinical tests. The sample may be taken from a subject suspected of having a disease or disorder. The sample may be taken from a subject experiencing unexplained symptoms, such as fatigue, nausea, weight loss, aches and pains, weakness, or bleeding. The sample may be taken from a subject having explained symptoms. The sample may be taken from a subject at risk of developing a disease or disorder due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

In some embodiments, a sample can be taken at a first time point and assayed, and then another sample can be taken at a subsequent time point and assayed. Such methods can be used, for example, for longitudinal monitoring purposes to track the development or progression of a disease. In some embodiments, the progression of a disease can be tracked before treatment, after treatment, or during the course of treatment, to determine the treatment's effectiveness. For example, a method as described herein can be performed on a subject prior to, and after, treatment with a lupus condition therapy to measure the disease's progression or regression in response to the lupus condition therapy.

After obtaining a sample from the subject, the sample may be processed to generate datasets indicative of a disease or disorder of the subject. For example, a presence, absence, or quantitative assessment of nucleic acid molecules of the sample at a panel of lupus condition-associated or interferon-associated genomic loci or may be indicative of a lupus condition of the subject. Processing the sample obtained from the subject may comprise (i) subjecting the sample to conditions that are sufficient to isolate, enrich, or extract a plurality of nucleic acid molecules, and (ii) assaying the plurality of nucleic acid molecules to generate the dataset (e.g., microarray data, nucleic acid sequences, or quantitative polymerase chain reaction (qPCR) data). Methods of assaying may include any assay known in the art or described in the literature, for example, a microarray assay, a sequencing assay (e.g., DNA sequencing, RNA sequencing, or RNA-Seq), or a quantitative polymerase chain reaction (qPCR) assay.

In some embodiments, a plurality of nucleic acid molecules is extracted from the sample and subjected to sequencing to generate a plurality of sequencing reads. The nucleic acid molecules may comprise ribonucleic acid (RNA) or deoxyribonucleic acid (DNA). The extraction method may extract all RNA or DNA molecules from a sample. Alternatively, the extraction method may selectively extract a portion of RNA or DNA molecules from a sample. Extracted RNA molecules from a sample may be converted to cDNA molecules by reverse transcription (RT).

The sample may be processed without any nucleic acid extraction. For example, the disease or disorder may be identified or monitored in the subject by using probes configured to selectively enrich nucleic acid (e.g., RNA or DNA) molecules corresponding to a panel of lupus condition-associated or interferon-associated genomic loci. The probes may be nucleic acid primers. The probes may have sequence complementarity with nucleic acid sequences from one or more of the panel of lupus condition-associated or interferon-associated genomic loci. The panel of lupus condition-associated or interferon-associated genomic loci may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more lupus condition-associated or interferon-associated genomic loci.

The probes may be nucleic acid molecules (e.g., RNA or DNA) having sequence complementarity with nucleic acid sequences (e.g., RNA or DNA) of one or more genomic loci (e.g., lupus condition-associated or interferon-associated genomic loci). These nucleic acid molecules may be primers or enrichment sequences. The assaying of the sample using probes that are selective for the one or more genomic loci (e.g., lupus condition-associated or interferon-associated genomic loci) may comprise use of array hybridization, polymerase chain reaction (PCR), or nucleic acid sequencing (e.g., RNA sequencing or DNA sequencing, such as RNA-Seq).

The assay readouts may be quantified at one or more genomic loci (e.g., lupus condition-associated or interferon-associated genomic loci) to generate the data indicative of the disease or disorder. For example, quantification of array hybridization or polymerase chain reaction (PCR) corresponding to a plurality of genomic loci (e.g., lupus condition-associated or interferon-associated genomic loci) may generate data indicative of the disease or disorder. Assay readouts may comprise quantitative PCR (qPCR) values, digital PCR (dPCR) values, digital droplet PCR (ddPCR) values, fluorescence values, etc., or normalized values thereof

Methods

Gene expression data may be compiled from SLE patients as follows. Data are derived from publicly available datasets and collaborators (Table 19). Differential gene expression (DE) may be performed for each dataset of SLE patients and controls. GCRMA normalized expression values are variance corrected using local empirical Bayesian shrinkage before calculation of DE using the ebayes function in the open source BioConductor LIMMA package (https://www.bioconductor.org/packages/release/bioc/html/limma.html). Resulting p-values are adjusted for multiple hypothesis testing and filtered to retain DE probes with an FDR<0.2. This cutoff is employed a priori to increase the number of genes that may be subsequently analyzed, with the understanding that even though the number of false positives may be increased, fewer false negatives may be excluded from the analysis. The heterogeneity in SLE patient blood samples may be demonstrated, and as a practical matter, signatures for LDGs and plasma cells are sometimes not detectable in limma analysis of populations depending on the specific patient make-up. An FDR of 0.2 may allow detection of cell types and processes which may not be found in all SLE patients, but that contribute significantly to the disease state in subpopulations of patients.

TABLE 19
SLE Datasets and SLE Time Course Datasets

| | | | | SLE | Healthy |
|---|---|---|---|---|---|
| | Sample | | | Pa- | Con- |
| | Type | Sex | SLEDAI | tients | trols |
| SLE Dataset | | | | | |
| GSE88884 | WB | Female | Six to 27 | 813 | 10^{b} |
| ILL1 | | | | | |
| GSE88884 | WB | Female | Six to 40 | 807 | 7^{b} |
| ILL2 | | | | | |
| GSE45291 | WB | Female | zero to 11 | 266 | 20 |
| GSE22098* | WB | Female | unknown | 24 | 15 |
| GSE61635 | WB | Female | unknown | 64 | 30 |
| GSE29536 | WB | Female | unknown | 27 | 41 |
| GSE39088 | WB | Female | Two to Ten | 17 | 34 |
| GSE49454* | WB | Female | Zero to 26 | 49 | 10 |
| GSE50772 | PBMC | Female | Zero to 13 | 56 | 20 |
| FDA PBMC | PBMC | Female | Zero to 25 | 30 | 6 |
| GSE38351 | CD14 | Female | Zero-24 | 12 | 12 |
| | Monocytes | | | | |
| GSE10325 | CD4 T cells | Female | Two-22 | 12 | 9 |
| GSE10325 | CD19 B | Female | Two-22 | 14 | 9 |
| | cells | | | | |
| GSE52471 | DLE | 5 | unknown | 7 | 10 |
| | | Female, | | | |
| | | 2 Male | | | |
| GSE72535 | DLE | 8 | Two | 9 | 9 |
| | | Female, | | | |
| | | 1 Male | | | |
| GSE36700^{a} | Synovium | Female | unknown | 4 | 4 |
| GSE32591 | Kidney Glom | Mixed | unknown | 30 | 14 |
| | Class II, | | | | |
| | III/IV | | | | |
| GSE32591 | Kidney TI | Mixed | unknown | 30 | 15 |
| | Class II, | | | | |
| | III/IV | | | | |
| SLE Time | | | | | |
| Course | | | | | |
| Datasets | | | | | |
| GSE72747 | WB | 9 | (Time 0) > 6 | 10 | 46^{c} |
| | | Female, | | | |
| | | 1 Male | | | |
| GSE88885 | WB | Female | (Time 0) > 6 | 86^{d} | 16 |
| GSE88886 | WB | Female | (Time 0) > 6 | 33^{d} | 12 |

*Only adult SLE patients were used
^{a}Osteoarthritis samples are the control synovial tissue
^{b}Used only female controls
^{c}No controls were available for this set. GSE39088 Male and Female controls were used for this dataset
^{d}Patients on standard of care (SOC) therapy who were given placebo in a clinical study
^{e}www.ncbi.nlm.nih.gov/geo/

Gene Set Variation Analysis (GSVA) may be performed as follows. The GSVA (V1.25.0) software package, an open source package available from R/Bioconductor, is used as a non-parametric, unsupervised method for estimating the variation of pre-defined gene sets in patient and control samples of microarray expression data sets (www.bioconductor.org/packages/release/bioc/html/GSVA.html). The inputs for the GSVA algorithm may be a gene expression matrix of log 2 microarray expression values and pre-defined gene sets co-expressed in SLE datasets. Enrichment scores (GSVA scores) may be calculated non-parametrically using a Kolmogorov Smirnoff (KS)-like random walk statistic and a negative value for a particular sample and gene set, meaning that the gene set has a lower expression than the same gene set with a positive value. The enrichment scores (ES) may be the largest positive and negative random walk deviations from zero, respectively, for a particular sample and gene set. The positive and negative ES for a particular gene set may depend on the expression levels of the genes that form the pre-defined gene set.

Random Group (Gr) 1 and Random Group (Gr) 2 signatures may be determined by first assigning random numbers to the list of DE genes (FDR 0.2) from dataset GSE49454 in Microsoft® Excel® using the formula “=rand( )”, and then sorting on ascending genes and taking the first 100 genes. This may be performed twice to generate Random Gr1 and Random Gr2 signatures. Gene symbols for these random signatures are listed in Tables 28-29.

TABLE 20
Genes with Induced Transcripts in PBMC by IFNA2 Treatment

| | | | | | | | | | |
|---|---|---|---|---|---|---|---|---|---|
| ACSL1 | CASP10 | CXCL11 | FLNA | IFI16 | ISG20 | MED1 | PDGFRL | SP110 | TOR1B |
| ADAR | CASP5 | CXCR2 | FOXO1 | IFI27 | ITIH2 | MGLL | PGGT1B | SP140 | TRA2B |
| ACT | CBR1 | CYP2J2 | FTL | IFI35 | JAK2 | CXCL9 | PKD2 | SPIB | TRD |
| AIM2 | CBWD1 | DAB2 | FUT4 | IFI44 | JUP | MMP16 | PLSCR1 | ST3GAL5 | TRIM21 |
| AKAP2 | CCL13 | DEFB1 | GADD45B | IFI44L | KCNA3 | MNDA | PMAIP1 | STAP1 | TRIM22 |
| APOBEC3B | CCL7 | DLL1 | GBAP1 | IFI6 | KDELR2 | MRPS15 | PML | STAT1 | TRIM26 |
| APOBEC3G | CCL8 | DSC2 | GBP1 | IFIT1 | KIF20B | MSR1 | PRKRA | STAT2 | TRLM38 |
| APOL3 | CCNA1 | DUSP5 | GBP2 | IFIT5 | KLF6 | MX1 | PSMB9 | STX11 | UBA7 |
| ATF3 | CCND2 | DUSP7 | GCH1 | IFITM1 | KPNB1 | MX2 | PTCH1 | SUPT3H | UBE2L6 |
| ATF5 | CD2AP | DYNLT1 | GCNT1 | IFITM2 | KRT8 | MYD88 | RBCK1 | SYN2 | UBE2S |
| BAG1 | CD38 | DYSF | GLB1 | IFITM3 | LAG3 | NAMPT | RET | TAF5L | UBE3A |
| BARD1 | CD4 | ECE1 | GLS | IFNG | LAMP3 | NFE2L3 | RGS1 | TAP1 | UNC93B1 |
| BCL7B | CD69 | EDN1 | GMPR | IFRD1 | LAP3 | NKTR | RGS6 | TAP2 | USP18 |
| BLVRA | CDC42EP1 | EIF2AK2 | GPR161 | IGL | LEPR | NMI | TRIM34 | TARBP1 | VAMP5 |
| BRCA1 | CDK4 | EIF2B1 | GUK1 | IKBKG | LGALS2 | NR3C1 | RPS9 | TCN2 | WARS |
| BRCA2 | CDKN1A | EIF4ENIF1 | HBG2 | IL15 | LGALS3BP | NUB1 | RTP4 | TFDP2 | WT1 |
| BST2 | CFB | ENPP2 | HCAR3 | IL15RA | LGALS9 | NUPR1 | SAT1 | TGM1 | XAF1 |
| BUB1 | CH25H | EPB41 | HIST2H2AA3 | IL1RN | LGMN | OAS1 | SCARB2 | TLR3 | |
| C2 | CHKA | ETV4 | HLA-DOA | IL6 | LMNB1 | OAS2 | SERPING1 | TLR7 | |
| CACNA1A | CNTN6 | F8 | HLA-DRB5 | INPPL1 | LMO2 | OAS3 | SIT1 | TNFRSF11A | |
| CAD | COL3A1 | FAF1 | HS6ST1 | IRF2 | LY6E | OSBPL1A | SLAMF1 | TNFSF10 | |
| CAMK2A | CTSL | FAS | HSP90AA1 | IRF7 | MAP2K5 | PATJ | SOCS1 | TNFSF6 | |
| CASP1 | CXCL10 | FGF1 | IDO1 | ISG15 | MCL1 | PDGFB | SP100 | TNK2 | |

TABLE 21
Genes with Induced Transcripts in PBMC by IFNB1 Treatment

| | | | | | | | | | |
|---|---|---|---|---|---|---|---|---|---|
| ACLY | CACNA1A | CHKA | ELF1 | HSP90AA1 | JAK2 | MFHAS1 | PKD2 | SFTPB | TNFAIP2 |
| ACSL1 | CAD | CISH | ELF4 | HSPA1A | JCHAIN | MGLL | PEEK | SIDT2 | TNFRSF11A |
| ADAM19 | CALD1 | CKB | ENPP2 | HSPA1L | JUP | CXCL9 | PLSCR1 | SIT1 | TNFSF10 |
| ADAP2 | CAMK2A | CMAHP | EPB41 | IDO1 | KCNA3 | MNDA | PMAIP1 | SLAMF1 | TNFSF6 |
| ADAR | CAPN2 | CNTN6 | ETV4 | IFI16 | KCNMB1 | MRPS15 | PML | SMO | TNK2 |
| ADGRE2 | CASP1 | CNTRL | ETV6 | IFI27 | KDELR2 | MS4A7 | PMS2 | SNX2 | TOR1B |
| ADM | CASP10 | COL3A1 | F8 | IFI35 | KIF20B | MSR1 | PPP2R2A | SOCS1 | TRA2B |
| AFF3 | CASP5 | COX17 | FAF1 | IFI44 | KLF2 | MX1 | PRKAG1 | SOS1 | TRD |
| ACT | CBR1 | CSF2RB | FAS | IFI6 | KLF6 | MX2 | PRKRA | SP100 | TRG |
| AIM2 | CBWD1 | CTSL | FBXW2 | IFIT1 | KLRB1 | MYD88 | PRKX | SP110 | TRIM21 |
| AKAP10 | CCL13 | CXCL10 | FCGR1A | IFIT5 | KPNB1 | NAMPT | PSMB8 | SP140 | TRIM22 |
| AKAP2 | CCL3L1 | CXCL11 | FCMR | IFITM1 | KRT8 | NAPSA | PSMB9 | SPIB | TRIM26 |
| ALOX12 | CCL4 | CXCL2 | FGF1 | IFITM2 | LAG3 | NBN | PTCH1 | SPTA1 | TRIM38 |
| ALOX5 | CCL7 | CXCR2 | FLNA | IFITM3 | LAMP3 | NCF1 | PTGER2 | SPTLC2 | TSPAN15 |
| ANXA4 | CCL8 | CYBB | FMR1 | IFNG | LANCL1 | NCOA2 | RALB | SRRM2 | TXK |
| APOBEC3B | CCNA1 | CYP19A1 | FOXO1 | IFRD1 | LAP3 | NEBL | RASGRP1 | SSB | UBA7 |
| APOBEC3G | CCND2 | CYP2J2 | FPR2 | IGL | LBR | NEK4 | RBBP6 | ST3GAL5 | UBE2L6 |
| APOL3 | CCR1 | DAB2 | FTL | IKBKE | LEPR | NFE2L3 | RBCK1 | STAP1 | UBE2S |
| ATF3 | CCR5 | DEFA1 | FUT4 | IKBKG | LGALS2 | NKTR | RERE | STAT1 | UBE3A |
| ATF5 | CCRL2 | DEFB1 | GADD45B | IL15 | LGALS3BP | NMI | RGS1 | STAT2 | UBQLN2 |
| ATM | CD163 | DHFR | GBAP1 | IL15RA | LGALS9 | NOTCH1 | RGS6 | STOML2 | UNC93B1 |
| ATP13A1 | CD164 | DLL1 | GBP1 | IL18BP | LGMN | NR3C1 | RIN1 | STX11 | USP15 |
| B4GAT1 | CD2AP | DMXL1 | GBP2 | IL18R1 | LILRA1 | NR4A3 | RIPK1 | SUPT3H | USP18 |
| BAG1 | CD38 | DNMT1 | GCH1 | IL1RN | LINC00597 | NUB1 | RIPK3 | TANK | USP25 |
| BAK1 | CD4 | DRAP1 | GCNT1 | IL6 | LMNB1 | NUPR1 | RIPOR2 | TAP1 | USPL1 |
| BARD1 | CD59 | DSC2 | GLS | IL7 | LMO2 | OAS1 | RNF114 | TAP2 | UVRAG |
| BCL11A | CD69 | DUSP5 | GMPR | INPP5D | LTA | OAS2 | TRIM34 | TAPBP | VAMP5 |
| BCL7B | CD72 | DUSP7 | GPI | INPPL1 | LTB4R | OAS3 | RPS6KA5 | TARBP1 | WARS |
| BGN | CD86 | DYNLT1 | GPR161 | IRF1 | LY6E | PATJ | RPS9 | TBX21 | WIPF1 |
| BLNK | CDK17 | DYSF | GUK1 | IRF2 | LYN | PAX5 | RRBP1 | TCN2 | WT1 |
| BLVRA | CDKN1A | E2F1 | HBG2 | IRF4 | MAP2K5 | PAX8 | RTP4 | TFDP2 | XAF1 |
| BLZF1 | CENPA | ECE1 | HCAR3 | IRF7 | MAP3K8 | PDE4B | SAT1 | TFF1 | ZNF107 |
| BRCA1 | CENPE | EDN1 | HHEX | IRF9 | MARCKS | PDGFB | SCARB2 | TGM1 | |
| BRCA2 | CFB | EGR1 | HIST2H2AA3 | ISG15 | MBNL | PDGFRL | SDS | THY1 | |
| BST2 | CFLAR | EIF2AK2 | HK2 | ISG20 | MCL1 | PFKFB3 | SELL | TLR1 | |
| BUB1 | CH25H | EIF2B1 | HLA-DOA | ITGAL | MED1 | PFKP | SERPIND1 | TLR3 | |
| C3AR1 | CHI3L2 | EIF4ENIF1 | HS6ST1 | ITGAX | MEF2A | PIM2 | SERPING1 | TLR7 | |

TABLE 22
Genes with Induced Transcripts in PBMC by IFNW1 Treatment

| | | | | | | | | | |
|---|---|---|---|---|---|---|---|---|---|
| ABCB10 | CAD | CFB | EIF4ENIF1 | GUK1 | IRF1 | MAP2K5 | OSBPL1A | SERPIND1 | TNFAIP3 |
| ACLY | CALD1 | CFLAR | ENPP2 | HBG2 | IRF2 | MARCKS | PATJ | SERPING1 | TNFRSF11A |
| ACSL1 | CAMK2A | CHKA | EPB41 | HHEX | IRF7 | MBNL1 | PAX8 | SFT2D2 | TNFSF10 |
| ADAR | CAPN2 | CKB | ERCC4 | HIST2H2AA3 | IRF8 | MCL1 | PDGFB | SIT1 | TNFSF6 |
| ADM | CASK | CMAHP | ETV4 | HLA-DOA | ISG15 | MED1 | PDGFRL | SLC30A4 | TNK2 |
| ACT | CASP1 | CNTN6 | ETV6 | HS6ST1 | ISG20 | MEF2A | PKD2 | SOCS1 | TOR1B |
| AIM2 | CASP10 | CNTRL | F8 | HSP90AA1 | ITIH2 | MGLL | PLEK | SOS1 | TRA2B |
| AKAP10 | CASP5 | COL3A1 | FAF1 | HSPA1A | JAK2 | CXCL9 | PLSCR1 | SP100 | TRD |
| AKAP2 | CBR1 | CSF2RB | FAS | IDO1 | JCHAIN | MLF1 | PMAIP1 | SP110 | TRIM21 |
| ALOX12 | CBWD1 | CTSL | FCER1G | IFI16 | JUP | MMP16 | PML | SP140 | TRIM22 |
| ANXA4 | CCL13 | CXCL10 | FGF1 | IF127 | KCNA3 | MNDA | PPP2R2A | SPIB | TRIM38 |
| APOBEC3B | CCL3L1 | CXCL11 | FGF13 | IFI35 | KDELR2 | MRPS15 | PRKAG1 | SRRM2 | UBA7 |
| APOBEC3G | CCL7 | CXCR2 | FGL2 | IFI44 | KIF20B | MS4A7 | PRKRA | ST3GAL5 | UBE2C |
| APOL3 | CCL8 | CYBB | FLNA | IFI6 | KLF6 | MSR1 | PSMB9 | STAP1 | UBE2L6 |
| ATF3 | CCNA1 | CYP19A1 | FMR1 | IFIT1 | KPNB1 | MX1 | PTCH1 | STAT1 | UBE2S |
| ATF5 | CCND2 | CYP2J2 | FOXO1 | IFIT5 | KRT8 | MX2 | PTGER2 | STAT2 | UNC93B1 |
| ATM | CCR1 | DEFB1 | FTL | IFITM1 | LAG3 | MYD88 | RALB | STX11 | USP18 |
| B4GAT1 | CCR5 | DLL1 | FUT4 | IFITM2 | LAMP3 | NAMPT | RBBP6 | SUPT3H | USP25 |
| BAG1 | CCR7 | DSC2 | GADD45B | IFITM3 | LAP3 | NCF1 | RBCK1 | TAP1 | WARS |
| BARD1 | CCRL2 | DUSP5 | GBAP1 | IFRD1 | LEPR | NFE2L3 | RERE | TAP2 | WIPF1 |
| BCL11A | CD164 | DUSP7 | GBP1 | IGL | LGALS2 | NKTR | RGS1 | TARBP1 | WT1 |
| BCL7B | CD2AP | DYNLT1 | GBP2 | IKBKG | LGALS3BP | NMI | RGS6 | TBX21 | XAF1 |
| BLVRA | CD38 | DYSF | GCH1 | IL15 | LGALS9 | NPTX1 | TRIM34 | TCN2 | ZNF107 |
| BLZF1 | CD4 | E2F1 | GCNT1 | IL15RA | LGMN | NR3C1 | RPS6KA5 | TFDP2 | |
| BRCA1 | CD47 | ECE1 | GLB1 | IL18R1 | LINC00597 | NUB1 | RTP4 | TFF1 | |
| BRCA2 | CD59 | EDN1 | GLS | IL1RN | LMNB1 | NUPR1 | SAT1 | TGM1 | |
| BRD4 | CD69 | EGR1 | GMPR | IL6 | LMO2 | OAS1 | SCARB2 | THY1 | |
| BST2 | CDKN1A | EIF2AK2 | GPR161 | IL7 | LY6E | OAS2 | SDS | TLR3 | |
| C3AR1 | CENPE | EIF2B1 | GSTM5 | INPPL1 | LYN | OAS3 | SELL | TLR7 | |

TABLE 23
Genes with Induced Transcripts in PBMC by IFNG Treatment

| | | | | | | | | | |
|---|---|---|---|---|---|---|---|---|---|
| ACLY | CASP10 | CXCL10 | FLII | IDO1 | KLF2 | NR3C1 | SERPIND1 | TAP1 | VSNL1 |
| ACSL1 | CCL8 | CXCL11 | GADD45B | IFI27 | LAP3 | OAS1 | SERPING1 | TAP2 | WARS |
| AFF2 | CCND2 | CYBB | GBP1 | IFI44 | LIMK2 | OAS3 | SFTPB | TBX21 | XRN1 |
| AIM2 | CCR5 | EDN1 | GBP2 | IL15 | LMNB1 | P2RY13 | SLAMF1 | TENM1 | |
| AKAP10 | CD38 | EPB41 | GCH1 | IL15RA | CXCL9 | PCDH9 | SLC1A5 | TFF1 | |
| APOL3 | CDKN1A | ETAA1 | GCNT1 | IL18BP | MMP25 | PLA2G4C | SOCS1 | TNFAIP2 | |
| ATF3 | CFB | ETV4 | GLS | IL1A | MRPS15 | PLEK | SP100 | TNFSF10 | |
| ATM | CKB | F8 | GSTM5 | IL7 | MSR1 | POLR2B | SPRY4 | UBD | |
| C1QB | CLEC10A | FAS | HBG2 | IRF1 | NET1 | PSMB9 | SRRM2 | UBE2C | |
| C4A | CPT1B | FBLN1 | HHEX | IRF8 | NIN | PTCH1 | STAT1 | UBE2L6 | |
| CALD1 | CSF2RB | FBXL2 | HP | JAK2 | NKTR | RALB | STAT2 | UBE3A | |
| CASP1 | CTNND2 | FCGR1A | ICAM1 | JCHAIN | NLRP1 | RGS1 | STX11 | VAMP5 | |

TABLE 24
Genes with Induced Transcripts in PBMC by IL12 Treatment

| | | | | | | | | | |
|---|---|---|---|---|---|---|---|---|---|
| ACLY | CASK | CYBB | FCGR1A | GZMB | IL18BP | KLF2 | NIN | SOCS1 | TNFAIP3 |
| AKAP10 | CASP1 | DEFA1 | GBP1 | HHEX | IL18R1 | KRT8 | NLRP1 | STAT1 | TNFSF10 |
| APOL3 | CCR5 | ETAA1 | GBP2 | HP | ILIA | LIMK1 | PCDH9 | TAP2 | TXK |
| BACH2 | CDKN3 | FASLG | GLS | HSPA6 | INPP5D | LINC00597 | SELL | TBX21 | |
| BRCA2 | CXCL10 | FBXL2 | GNPDA1 | IFNG | INSIG1 | LY75 | SERPIND1 | TFF1 | |
| CALD1 | CXCR3 | FCER2 | GSTM5 | IL16 | IRF1 | MMP25 | SLAMF1 | TNFAIP2 | |

TABLE 25
Genes with Induced Transcripts in PBMC by TNF Treatment

| | | | | | | | | | |
|---|---|---|---|---|---|---|---|---|---|
| ACLY | BHMT | CDKN3 | EPB41 | GJB2 | IL16 | MAP3K4 | NFKBIA | RPGR | TAP2 |
| ACSL1 | BIRC3 | CKB | EREG | GLS | IL18 | MARCKS | NFKBIZ | RPS9 | TBX3 |
| ADGRE2 | BRCA1 | CR2 | ETAA1 | GMIP | IL1A | MGLL | NKX3-2 | SDC4 | TFF1 |
| AK3 | CALD1 | CTNND2 | F3 | GP1BA | IL1B | MMP19 | NR3C1 | SERPIND1 | TNF |
| AKAP10 | CASP1 | CXCL1 | FABP1 | GRK3 | IL1RN | MN1 | OAS3 | SFRP1 | TNFAIP2 |
| AMPD3 | CASP10 | CXCL2 | FBXL2 | HCAR3 | IL6 | MRPS15 | PATJ | SH3BP5 | TNFAIP3 |
| APOL3 | CCL15 | CXCL3 | FCER2 | HHEX | INHBA | MSC | PDE4DIP | SLAMF1 | TNFRSF11A |
| ARID3A | CCL20 | CXCL8 | FCGR2A | HOMER2 | INSIG1 | MTF1 | PDPN | SLC30A4 | TRAF1 |
| ARSE | CCL23 | CYP27B1 | FLJ11129 | HP | ITGA6 | MX1 | PIAS4 | SOD2 | TSC22D1 |
| ASAP1 | CCL3L1 | DAB2 | FLNA | ICAM1 | KITLG | NAMPT | PLAUR | SPI1 | TYROBP |
| B4GALT5 | CD37 | EBI3 | G0S2 | IDO1 | KLF1 | NELL2 | PTGES | SSPN | UBE2C |
| BCL2A1 | CD38 | EGR1 | GBP1 | IFI44 | KMO | NFKB1 | PTGS2 | STAT4 | VEGFA |
| BHLHE41 | CD83 | EGR2 | GCH1 | IKBKG | LGALS3BP | NFKB2 | RELB | TAF15 | WT1 |

TABLE 26
Genes of IFN Core with Induced Transcripts

| | | | | | | | | | |
|---|---|---|---|---|---|---|---|---|---|
| ACSL1 | CASP10 | CXCL11 | FAF1 | HS6ST1 | INPPL1 | LGMN | NUPR1 | SERPING1 | TLR7 |
| ADAR | CASP5 | CXCL9 | FAS | HSP90AA1 | IRF2 | LMNB1 | OAS1 | SIT1 | TNFRSF11A |
| ACT | CBR1 | CXCR2 | FGF1 | IDO1 | IRF7 | LMO2 | OAS2 | SOCS1 | TNFSF10 |
| AIM2 | CBWD1 | CYP2J2 | FLNA | IFI16 | ISG15 | LY6E | OAS3 | SP100 | TNFSF6 |
| AKAP2 | CCL13 | DEFB1 | FOXO1 | IFI27 | ISG20 | MAP2K5 | PATJ | SP110 | TNK2 |
| APOBEC3B | CCL7 | DLL1 | FTL | IFI35 | JAK2 | MCL1 | PDGFB | SP140 | TOR1B |
| APOBEC3G | CCL8 | DSC2 | FUT4 | IFI44 | JUP | MED1 | PDGFRL | SPIB | TRA2B |
| APOL3 | CCNA1 | DUSP5 | GADD45B | IFI6 | KCNA3 | MGLL | PKD2 | ST3GAL5 | TRD |
| ATF3 | CCND2 | DUSP7 | GBAP1 | IFIT1 | KDELR2 | MNDA | PLSCR1 | STAP1 | TRIM21 |
| ATF5 | CD2AP | DYNLT1 | GBP1 | IFIT5 | KIF20B | MRPS15 | PMAIP1 | STAT1 | TRIM22 |
| BAG1 | CD38 | DYSF | GBP2 | IFITM1 | KLF6 | MSR1 | PML | STAT2 | TRIM34 |
| BARD1 | CD4 | ECE1 | GCH1 | IFITM2 | KPNB1 | MX1 | PRKRA | STX11 | TRLM38 |
| BCL7B | CD69 | EDN1 | GCNT1 | IFITM3 | KRT8 | MX2 | PSMB9 | SUPT3H | UBA7 |
| BLVRA | CDKN1A | EIF2AK2 | GLS | IFRD1 | LAG3 | MYD88 | PTCH1 | TAP1 | UBE2L6 |
| BRCA1 | CFB | EIF2B1 | GMPR | IGL | LAMP3 | NAMPT | RBCK1 | TAP2 | UBE2S |
| BRCA2 | CHKA | EIF4ENIF1 | GPR161 | IKBKG | LAP3 | NFE2L3 | RGS1 | TARBP1 | UNC93B1 |
| BST2 | CNTN6 | ENPP2 | GUK1 | IL15 | LEPR | NKTR | RGS6 | TCN2 | USP18 |
| CAD | COL3A1 | EPB41 | HBG2 | IL15RA | LGALS2 | NMI | RTP4 | TFDP2 | WARS |
| CAMK2A | CTSL | ETV4 | HIST2H2AA3 | IL1RN | LGALS3BP | NR3C1 | SAT1 | TGM1 | WT1 |
| CASP1 | CXCL10 | F8 | HLA-DOA | IL6 | LGALS9 | NUB1 | SCARB2 | TLR3 | XAF1 |

TABLE 27
Genes of Type I and Type II IFN Core

| | | | | | | | | |
|---|---|---|---|---|---|---|---|---|
| ACSL1 | CCL8 | CXCL11 | GBP1 | IDO1 | LAP3 | NR3C1 | SERPING1 | TAP1 |
| AIM2 | CCND2 | EDN1 | GBP2 | IFI27 | LMNB1 | OAS1 | SOCS1 | TAP2 |
| APOL3 | CD38 | EPB41 | GCH1 | IFI44 | CXCL9 | OAS3 | SP100 | FAS |
| ATF3 | CDKN1A | ETV4 | GCNT1 | IL15 | MRPS15 | PSMB9 | STAT1 | TNFSF10 |
| CASP1 | CFB | F8 | GLS | IL15RA | MSR1 | PTCH1 | STAT2 | UBE2L6 |
| CASP10 | CXCL10 | GADD45B | HBG2 | JAK2 | NKTR | RGS1 | STX11 | WARS |

TABLE 28
Genes of Random Gr 1

| | | | | | | | | | |
|---|---|---|---|---|---|---|---|---|---|
| TYW3 | AASDHPPT | HNRNPC | MS2P1 | FAM50A | PSME3 | RAB13 | SNTB1 | WDR45 | KDM6B |
| PID1 | LOC284023 | NPC1 | ZC3H8 | EEF2K | PPP1R35 | APH1B | USB1 | SLC2A5 | ST6GALNAC4 |
| MXD4 | EEF2 | ANAPC10 | HNRNPR | FAM175B | AKTIP | SPPL2A | NCOA1 | DGAT2 | APOPT1 |
| ARPC4 | HIC2 | ZNF362 | IDH3B | ZNF485 | RNF4 | BRCA1 | RHOT1 | CYP4F3 | CASP5 |
| CD81 | SSR2 | WDR82 | HPS5 | MCM7 | FAM189B | DOCK8 | DLST | PFKFB4 | CDC34 |
| TPM3 | ZNF830 | PRPF8 | KRT10 | DHX32 | YWHAE | DGKD | KIAA0513 | ABCG1 | EIF5 |
| TBC1D31 | FAM84B | RASSF1 | MIEF1 | NDUFC1 | PAM16 | NFKBIA | ATP6V0B | CARD16 | ACO1 |
| ASF1A | UTP23 | EIF5 | RNF144A | ACO1 | TARBP1 | STAU1 | FCER1G | MARCH2 | RBM4 |
| HMGN2 | MIB2 | MIS12 | NMD3 | FASTKD2 | CCNA2 | RELB | ABCA7 | ACOX1 | RABGAP1L |
| SNX1 | TMEM177 | RPL15 | SF3B4 | GID8 | SETX | SLC43A3 | GYG1 | PDLIM7 | MGAT1 |

TABLE 29
Genes of Random Gr 2

| | | | | | | | | | |
|---|---|---|---|---|---|---|---|---|---|
| SH3YL1 | BRIX1 | FAM159A | SECISBP2L | VDAC3 | ZNF3 | SAP30L | ZNF493 | ACTA2 | PELI1 |
| TARP | FBXO21 | SLC2A4RG | ALKBH2 | SLC30A5 | AUH | MANBAL | TAZ | CTRL | FAM214B |
| VPS51 | PEBP1 | DDX1 | UBE2N | ZNF275 | ANAPC15 | FAM45A | RAP2C | TMEM170B | SLC2A3 |
| ARL2BP | PAOX | PHF5A | SLC3A2 | PHF10 | TNPO2 | ATP11B | RAB32 | ABCA7 | TRIB1 |
| RPS28 | JADE1 | VKORC1 | CEP41 | ACD | FAM192A | RBM10 | GPR1 | TMEM120A | COLGALT1 |
| NSA2 | POGLUT1 | PSMC5 | LYPLAL1 | HSCB | SCLT1 | SPTBN4 | RNU4-2 | PKM | STAT3 |
| ACYP2 | DENND2D | MAEA | OXCT1 | ZNF485 | PI4KB | SPAG9 | LRWD1 | NAMPT | MPO |
| SPOUT1 | TMEM8B | KDSR | RANGAP1 | PPP1R11 | CALML4 | PTTG1IP | LATP4B | MSL1 | OLR1 |
| HIVEP2 | EXOSC1 | FKBP4 | SRSF4 | MCM7 | C4orf32 | PRELID1 | LILRB3 | ACSL4 | PSMD1 |
| SDR39U1 | TMEM14B | LINC01278 | NENF | RPUSD1 | CCNA2 | GGA3 | MYADM | ZDHHC19 | MAP3K3 |

Enrichment modules containing cell type and process specific genes may be created through an iterative process of identifying DE transcripts pertaining to a restricted profile of hematopoietic cells in a majority of the SLE microarray datasets analyzed and checked for expression in purified T cells, B cells, and monocytes to remove transcripts indicative of multiple cell types. Transcripts may be researched by searching through literature. In the case of the cell cycle, unfolded protein response (UPR), and plasma cell modules, genes may be initially identified through the DE analysis, and WGCNA created modules may correlated to SLEDAI from CD19 and CD20 B cells. These genes may be identified by searching through literature, and STRING interactome analysis as belonging to these categories and their DE may be confirmed in the 13 SLE WB and PBMC datasets used in these studies.

In order to have a significant overlap, a minimum number, such as three transcripts, for each category may have to be found in each dataset and may be used based on calculating an error rate of 20% for one transcript, an error rate of 4% for two transcripts, and an error rate of 0.8% for three transcripts. GSVA enrichment modules used for linear regression analyses may have overlapping transcripts between the IFN signatures and the cell type specific signatures removed.

For each group of patients and controls analyzed by GSVA, DE may be performed on active and inactive patients together relative to HC at an FDR of 0.2. Differences between HC and SLE patient GSVA enrichment scores may be determined using the Welch's t-test for unequal variances (e.g., in PRISM 7.0 v7.0c). In order to quantitate the difference between the SLE and HC groups, the Hedge's g effect size may be determined (e.g., using the Effect Size Calculator for T-Test at the website Social Science Statistics, www.socscistatistics.com/effectsize/Default3.aspx).

Z score analysis may be performed as follows. Z score calculations may be employed to identify and compare the enrichment of specific signatures in SLE and control datasets. For each regulator, an activation z-score may be calculated strictly from the experimentally observed information provided for the downstream targets. Reference datasets may be used to determine the identity and direction (increased or decreased) of downstream targets. The formula Z=x/σₓ=Σᵢwᵢxᵢ/√Σᵢwᵢ² may be used to calculate Z scores with edge weights set to 1. Z scores above or below 1.96 are significant at the 95% confidence level, and Z scores above or below 2.54 are significant at the 99% confidence level. SLE WB and PBMC datasets may be divided into patients with SLEDAI≥6 (active) and patients with SLEDAI<6 (inactive).

Reference and control datasets may be obtained as follows. A first reference dataset used may comprise the transcripts (FDR<0.01, LFC>2) from the in vitro treatment of healthy, human PBMC with 0.6 pM IFNA2b, IFNB1a, IFNW1, IFNG, IL12, or TNF differentially expressed compared to control treated PBMC. To eliminate differences in genetic background, a single donor may be used for these experiments. A second reference dataset used may comprise the IFNB1 (MS-IFNB1) signature induced in vivo in the whole blood of a first plurality of Multiple Sclerosis (MS) patients treated with IFNB1 (Avonex, Betaseron, or Rebif) for one to two years compared to a second plurality of MS patients not treated with IFNB1. A third reference dataset used may comprise the IFNA signature induced in a plurality of HepC patients treated with recombinant IFNA for six hours compared to their PBMC before the injection of recombinant IFNA (as described in Table 2 of [Hoffman, R. W. et al. Gene Expression and Pharmacodynamic Changes in 1,760 Systemic Lupus Erythematosus Patients From Two Phase III Trials of BAFF Blockade With Tabalumab. Arthritis Rheumatol. 69, 643-654 (2017)], which is hereby incorporated by reference in its entirety) for the HepC-IFNA2 signature. Published transcripts of PBMC from patients with sepsis DE to controls, and of skin biopsies from patients with dermatomyositis DE to controls may be used as comparators for Z score calculations. The reference dataset for the alternative IFNB1 signaling pathway may be taken from the IFNB1-induced signatures in IFNAR1-deficient mice. Genes may be translated to human gene symbols, and the increased transcripts may be used to determine GSVA scores.

Weighted Gene Co-expression Network Association (WGCNA) may be performed as follows. WGCNA, an open source package for R available at https://horvath.genetics.ucla.edu/html/CoexpressionNetwork/Rpackages/WGCNA/, may be used. Log 2 normalized microarray expression values for WB, PBMC, purified T cell, B cell, or monocyte datasets may be filtered using an IQR to remove saturated probes with low variability between samples and used as inputs to WGCNA (V1.51). Adjacency co-expression matrices for all probes in a given set may be calculated by Pearson's correlation using signed network type specific formulae. Blockwise network construction may be performed using soft threshold power values that are manually selected and specific to each dataset in order to preserve maximal scale free topology of the networks. Resultant dendrograms of correlation networks may be trimmed to isolate individual modular groups of probes, labeled using semi-random color assignments, based on a detection cut height of 1, with a merging cut height of 0.2, with the additional use of a partitioning around medoids function. Final membership of probes representing the same gene into modules may be based on selection of greatest scale within module correlation against module eigengene (ME) values. Correlation to the presence of SLE disease (versus control) or the disease measure SLEDAI may be performed using Pearson's r against MEs, defining modules as either positively or negatively correlated with those traits as a whole.

F Test analysis for DE gene expression in SLE patients with multiple time points may be performed as follows. One-way analysis of variance (ANOVA) may be used to compare means of two or more samples (using the F distribution). The statistic fit2$F and the corresponding fit$F.p.value may be used to combine the pair-wise comparisons into one F-test. This is equivalent to a one-way ANOVA for each gene, except that the residual mean squares have been moderated between genes. For the GSE88885 dataset, a subset of patients on standard of care (SOC) therapy and placebo from the Illuminate 1 clinical trial have time-course microarray expression data; 86 placebo treated SLE patients at t=0, t=16 weeks, and t=52 weeks and 16 HC may be analyzed together. For GSE88886, a subset of placebo patients on SOC from the Illuminate 2 clinical trial with time-course microarray data, 33 placebo treated SLE patients with time points at t=0, t=16 weeks, and t=52 weeks and 12 HC may be analyzed together. For GSE72747, all ten patient values at t=0, t=12 weeks, and t=24 weeks and 46 HC from GSE39088 may be analyzed together. Significant changes in IGS may be determined to be a standard deviation (SD) of 0.2 by calculating the SD of the HC for each signature and using the highest SD as a measure of significance.

Other statistical analyses may be performed as follows. GraphPad PRISM 7 version 7.0c may be used to perform linear regression analysis, calculation of r² values, and Tukey's multiple comparison analysis for ANOVA. Average and SD may be calculated using Microsoft® Excel®. The built-in ANOVA function in R may be used to compute two-way ANOVA p-values.

In some embodiments, the systems and methods herein are configured for RNA sequencing (RNA-Seq) data analysis, especially single-cell RNA-Seq (scRNA-Seq) data analysis. In some embodiments, scRNA-Seq data has the potential to increase our understanding of cell populations in various diseases, such as lupus and cancer. However, phenotype of individual cells may not be available or manageable when the cell population is large, e.g., 10,000 cells. In some embodiments, scRNA-Seq data is used to identify cell populations or clusters computationally.

In some embodiments, the RNA-Seq data comprises data entries of gene expression levels. In some embodiments, the RNA-Seq data is generated using unique molecular identifiers (UMIs). In some embodiments, the RNA-Seq data is not generated using UMIs. In some embodiments, the RNA-Seq data is of each single cell of the plurality of cells, e.g., scRNA-Seq data. In some embodiments, the RNA-Seq data of one or more cells of the plurality of cells comprise data entries that are identical to the data entries in other cells of the plurality of cells. In some embodiments, the identical data entries is more than 50%, 60%, 70%, 80%, 90%, or even more of the RNA-Seq data of the one or more cells. As an example, data sets generated using UMI can have the vast majority (e.g., 90-95%) of data entries set to zero, which baffles existing bioinformatics techniques and even those designed for use with bulk RNA-Seq data. Such large number of zero entries tends to make all cells look alike in experiments intended to study cellular heterogeneity.

In some embodiments, the RNA-Seq data is raw gene expression data. In some embodiments, the RNA-Seq data for each cell includes one data entry for each gene, the data entry can range from zero to an arbitrary number that is greater than zero, e.g., 10, 100, 1,000, 10,000, etc.

In some embodiments, each cell is associated with a unique cell identification number (ID). In some embodiments, the scRNA-Seq data of a cell is associated with the unique cell ID.

### Classifiers

In some embodiments, the present disclosure provides a system, method, or kit having data analysis realized in software application, computing hardware, or both. In various embodiments, the analysis application or system includes at least a data receiving module, a data pre-processing module, a data analysis module, a data interpretation module, or a data visualization module. In one embodiment, the data receiving module can comprise computer systems that connect laboratory hardware or instrumentation with computer systems that process laboratory data. In one embodiment, the data pre-processing module can comprise hardware systems or computer software that performs operations on the data in preparation for analysis. Examples of operations that can be applied to the data in the pre-processing module include affine transformations, denoising operations, data cleaning, reformatting, or subsampling. A data analysis module, which can be specialized for analyzing genomic data from one or more genomic materials, can, for example, take assembled genomic sequences and perform probabilistic and statistical analysis to identify abnormal patterns related to a disease, pathology, state, risk, condition, or phenotype. A data interpretation module can use analysis methods, for example, drawn from statistics, mathematics, or biology, to support understanding of the relation between the identified abnormal patterns and health conditions, functional states, prognoses, or risks. A data visualization module can use methods of mathematical modeling, computer graphics, or rendering to create visual representations of data that can facilitate the understanding or interpretation of results.

Feature sets may be generated from datasets obtained using one or more assays of a biological sample, and a trained algorithm may be used to process one or more of the feature sets to identify or assess the condition (e.g., a disease or disorder, such as a lupus condition). For example, the trained algorithm may be used to apply a machine learning classifier to a plurality of lupus condition-associated or interferon-associated genomic loci that are associated with two or more classes of individuals inputted into a machine learning model, in order to classify a subject into one of the two or more classes of individuals. For example, the trained algorithm may be used to apply a machine learning classifier to a plurality of lupus condition-associated or interferon-associated genomic loci that are associated with individuals with known conditions (e.g., a disease or disorder, such as a lupus condition) and individuals not having the condition (e.g., healthy individuals, or individuals who do not have a lupus condition), in order to classify a subject as having the condition (e.g., positive test outcome) or not having the condition (e.g., negative test outcome).

The trained algorithm may be configured to identify the presence (e.g., positive test result) or absence (e.g., negative test result) of one or more conditions (e.g., a disease or disorder, such as a lupus condition) with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99%. This accuracy may be achieved for a set of at least about 25, at least about 50, at least about 100, at least about 150, at least about 200, at least about 250, at least about 300, at least about 350, at least about 400, at least about 450, at least about 500, at least about 1,000, or more than about 1,000 independent samples.

The trained algorithm may comprise a machine learning algorithm, such as a supervised machine learning algorithm. The supervised machine learning algorithm may comprise, for example, a Random Forest, a support vector machine (SVM), a neural network, or a deep learning algorithm. The trained algorithm may comprise a classification and regression tree (CART) algorithm. The trained algorithm may comprise an unsupervised machine learning algorithm.

The trained algorithm may comprise a classifier configured to accept as input a plurality of input variables or features (e.g., lupus condition-associated or interferon-associated genomic loci) and to produce or output one or more output values based on the plurality of input variables or features (e.g., lupus condition-associated or interferon-associated genomic loci). The plurality of input variables or features may comprise one or more datasets indicative of the presence (e.g., positive test result) or absence (e.g., negative test result) of one or more conditions (e.g., a disease or disorder, such as a lupus condition). For example, an input variable or feature may comprise a number of sequences corresponding to or aligning to each of the plurality of lupus condition-associated or interferon-associated genomic loci.

The plurality of input variables or features may also include clinical information of a subject, such as health data. For example, the health data of a subject may comprise one or more of: a diagnosis of one or more conditions (e.g., a disease or disorder, such as a lupus condition), a prognosis of one or more conditions (e.g., a disease or disorder, such as a lupus condition), a risk of having one or more conditions (e.g., a disease or disorder, such as a lupus condition), a treatment history of one or more conditions (e.g., a disease or disorder, such as a lupus condition), a history of previous treatment for one or more conditions (e.g., a disease or disorder, such as a lupus condition), a history of prescribed medications, a history of prescribed medical devices, age, height, weight, sex, smoking status, and one or more symptoms of the subject. For example, the disease or disorder may comprise one or more of: systemic lupus erythematosus (SLE), discoid lupus erythematosus (DLE), and lupus nephritis (LN).

The trained algorithm may comprise a classifier, such that each of the one or more output values comprises one of a fixed number of possible values (e.g., a linear classifier, a logistic regression classifier, etc.) indicating a classification of the sample by the classifier. The trained algorithm may comprise a binary classifier, such that each of the one or more output values comprises one of two values (e.g., {0, 1}, {positive, negative}, or {high-risk, low-risk}) indicating a classification of the sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the sample by the classifier.

The classifier may be configured to classify samples by assigning output values, which may comprise descriptive labels, numerical values, or a combination thereof. Some of the output values may comprise descriptive labels. Such descriptive labels may provide an identification or indication of the presence (e.g., positive test result) or absence (e.g., negative test result) of one or more conditions (e.g., a disease or disorder, such as a lupus condition) of the subject, and may comprise, for example, positive, negative, high-risk, intermediate-risk, low-risk, or indeterminate. Such descriptive labels may provide an identification of a treatment for the one or more conditions of the subject, and may comprise, for example, a therapeutic intervention, a duration of the therapeutic intervention, and/or a dosage of the therapeutic intervention suitable to treat the one or more conditions of the subject. Such descriptive labels may provide an identification of secondary clinical tests that may be appropriate to perform on the subject, and may comprise, for example, an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof. For example, such descriptive labels may provide a prognosis of the one or more conditions of the subject. As another example, such descriptive labels may provide a relative assessment of the one or more conditions of the subject. Some descriptive labels may be mapped to numerical values, for example, by mapping “positive” to 1 and “negative” to 0.

The classifier may be configured to classify samples by assigning output values that comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the one or more conditions (e.g., a disease or disorder, such as a lupus condition) of the subject. Some numerical values may be mapped to descriptive labels, for example, by mapping 1 to “positive” and 0 to “negative.”

The classifier may be configured to classify samples by assigning output values based on one or more cutoff values. For example, a binary classification of samples may assign an output value of “positive” or 1 if the sample indicates that the subject has at least a 50% probability of having one or more conditions (e.g., a disease or disorder, such as a lupus condition), thereby assigning the subject to a class of individuals receiving a positive test result. As another example, a binary classification of samples may assign an output value of “negative” or 0 if the sample indicates that the subject has less than a 50% probability of having one or more conditions (e.g., a disease or disorder), thereby assigning the subject to a class of individuals receiving a negative test result. In this case, a single cutoff value of 50% is used to classify samples into one of the two possible binary output values or classes of individuals (e.g., those receiving a positive test result and those receiving a negative test result). Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, the classifier may be configured to classify samples by assigning an output value of “positive” or 1 if the sample indicates that the subject has a probability of having one or more conditions (e.g., a disease or disorder, such as a lupus condition) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of having one or more conditions (e.g., a disease or disorder, such as a lupus condition) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classifier may be configured to classify samples by assigning an output value of “negative” or 0 if the sample indicates that the subject has a probability of having one or more conditions (e.g., a disease or disorder, such as a lupus condition) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having one or more conditions (e.g., a disease or disorder, such as a lupus condition) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classifier may be configured to classify samples by assigning an output value of “indeterminate” or 2 if the sample is not classified as “positive”, “negative”, 1, or 0. In this case, a set of two cutoff values is used to classify samples into one of the three possible output values or classes of individuals (e.g., corresponding to outcome groups of individuals having “low risk,” “intermediate risk,” and “high risk” of having one or more conditions, such as a disease or disorder). Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values or classes of individuals, where n is any positive integer.

The trained algorithm may be trained with a plurality of independent training samples. Each of the independent training samples may comprise a sample from a subject, associated datasets obtained by assaying the sample (as described elsewhere herein), and one or more known output values or classes of individuals corresponding to the sample (e.g., a clinical diagnosis, prognosis, absence, or treatment efficacy of a condition of the subject). Independent training samples may comprise samples and associated datasets and outputs obtained or derived from a plurality of different subjects. Independent training samples may comprise samples and associated datasets and outputs obtained at a plurality of different time points from the same subject (e.g., on a regular basis such as weekly, biweekly, or monthly), as part of a longitudinal monitoring of a subject before, during, and after a course of treatment for one or more conditions of the subject. Independent training samples may be associated with presence of the condition (e.g., training samples comprising samples and associated datasets and outputs obtained or derived from a plurality of subjects known to have the condition). Independent training samples may be associated with absence of the condition (e.g., training samples comprising samples and associated datasets and outputs obtained or derived from a plurality of subjects who are known to not have a previous diagnosis of the condition or who have received a negative test result for the condition).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise samples associated with presence of the condition and/or samples associated with absence of the condition. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the condition (e.g., a disease or disorder, such as a lupus condition). The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with absence of the condition (e.g., a disease or disorder, such as a lupus condition). In some embodiments, the sample is independent of samples used to train the trained algorithm.

The trained algorithm may be trained with a first number of independent training samples associated with a presence of the condition (e.g., a disease or disorder, such as a lupus condition) and a second number of independent training samples associated with an absence of the condition (e.g., a disease or disorder, such as a lupus condition). The first number of independent training samples associated with presence of the condition (e.g., a disease or disorder, such as a lupus condition) may be no more than the second number of independent training samples associated with absence of the condition (e.g., a disease or disorder, such as a lupus condition). The first number of independent training samples associated with a presence of the condition (e.g., a disease or disorder) may be equal to the second number of independent training samples associated with an absence of the condition (e.g., a disease or disorder, such as a lupus condition). The first number of independent training samples associated with a presence of the condition (e.g., a disease or disorder, such as a lupus condition) may be greater than the second number of independent training samples associated with an absence of the condition (e.g., a disease or disorder, such as a lupus condition).

The trained algorithm may comprise a classifier configured to identify the presence (e.g., positive test result) or absence (e.g., negative test result) of one or more conditions (e.g., a disease or disorder, such as a lupus condition) at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the presence (e.g., positive test result) or absence (e.g., negative test result) of the one or more conditions by the trained algorithm may be calculated as the percentage of independent test samples (e.g., subjects known to have the condition or subjects with negative clinical test results for the condition) that are correctly identified or classified as having or not having the condition.

The trained algorithm may comprise a classifier configured to identify one or more conditions (e.g., a disease or disorder, such as a lupus condition) with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the condition using the trained algorithm may be calculated as the percentage of samples identified or classified as having the condition that correspond to subjects that truly have the condition.

The trained algorithm may comprise a classifier configured to identify one or more conditions (e.g., a disease or disorder, such as a lupus condition) with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the condition using the trained algorithm may be calculated as the percentage of samples identified or classified as not having the condition that correspond to subjects that truly do not have the condition.

The trained algorithm may comprise a classifier configured to identify one or more conditions (e.g., a disease or disorder, such as a lupus condition) with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the condition using the trained algorithm may be calculated as the percentage of independent test samples associated with presence of the condition (e.g., subjects known to have the condition) that are correctly identified or classified as having the condition.

The trained algorithm may comprise a classifier configured to identify one or more conditions (e.g., a disease or disorder, such as a lupus condition) with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the condition using the trained algorithm may be calculated as the percentage of independent test samples associated with absence of the condition (e.g., subjects with negative clinical test results for the condition) that are correctly identified or classified as not having the condition.

The trained algorithm may comprise a classifier configured to identify the presence (e.g., positive test result) or absence (e.g., negative test result) of one or more conditions (e.g., a disease or disorder, such as a lupus condition) with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying samples as having or not having the condition.

Classifiers of the trained algorithm may be adjusted or tuned to improve or optimize one or more performance metrics, such as accuracy, PPV, NPV, clinical sensitivity, clinical specificity, AUC, or a combination thereof (e.g., a performance index incorporating a plurality of such performance metrics, such as by calculating a weight sum therefrom), of identifying the presence (e.g., positive test result) or absence (e.g., negative test result) of the condition. The classifiers may be adjusted or tuned by adjusting parameters of the classifiers (e.g., a set of cutoff values used to classify a sample as described elsewhere herein, or weights of a neural network) to improve or optimize the performance metrics. The one or more classifiers may be adjusted or tuned so as to reduce an overall classification error (e.g., an “out-of-bag” or oob error rate for a Random Forest classifier). The one or more classifiers may be adjusted or tuned continuously during the training process (e.g., as sample datasets are added to the training set) or after the training process has completed.

The trained algorithm may comprise a plurality of classifiers (e.g., an ensemble) such that the plurality of classifications or outcome values of the plurality of classifiers may be combined to produce a single classification or outcome value for the sample. For example, a sum or a weighted sum of the plurality of classifications or outcome values of the plurality of classifiers may be calculated to produce a single classification or outcome value for the sample. As another example, a majority vote of the plurality of classifications or outcome values of the plurality of classifiers may be identified to produce a single classification or outcome value for the sample. In this manner, a single classification or outcome value may be produced for the sample having greater confidence or statistical significance than the individual classifications or outcome values produced by each of the plurality of classifiers.

After the trained algorithm is initially trained, a subset of the inputs may be identified as most influential or most important to be included for making high-quality classifications (e.g., having highest permutation feature importance). For example, a subset of the panel of lupus condition-associated or interferon-associated genomic loci may be identified as most influential or most important to be included for making high-quality classifications or identifications of conditions (or sub-types of conditions). The panel of lupus condition-associated or interferon-associated genomic loci, or a subset thereof, may be ranked based on classification metrics indicative of each influence or importance of each individual lupus condition-associated or interferon-associated genomic locus toward making high-quality classifications or identifications of conditions (or sub-types of conditions). Such metrics may be used to reduce, in some cases significantly, the number of input variables (e.g., predictor variables) that may be used to train the one or more classifiers of the trained algorithm to a desired performance level (e.g., based on a desired minimum accuracy, PPV, NPV, clinical sensitivity, clinical specificity, AUC, or a combination thereof).

For example, if training a classifier of the trained algorithm with a plurality comprising several dozen or hundreds of input variables to the classifier results in an accuracy of classification of more than 99%, then training the classifier of the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

As another example, if training a classifier of the trained algorithm with a plurality comprising several dozen or hundreds of input variables to the classifier results in a sensitivity or specificity of classification of more than 99%, then training the classifier of the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable sensitivity or specificity of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

The subset of the plurality of input variables (e.g., the panel of lupus condition-associated or interferon-associated genomic loci) to the classifier of the trained algorithm may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics (e.g., permutation feature importance).

Upon identifying the subject as having one or more conditions (e.g., a disease or disorder, such as a lupus condition), the subject may be optionally provided with a therapeutic intervention (e.g., prescribing an appropriate course of treatment to treat the one or more conditions of the subject). The therapeutic intervention may comprise a prescription of an effective dose of a drug, a further testing or evaluation of the condition, a further monitoring of the condition, or a combination thereof. If the subject is currently being treated for the condition with a course of treatment, the therapeutic intervention may comprise a subsequent different course of treatment (e.g., to increase treatment efficacy due to non-efficacy of the current course of treatment).

The therapeutic intervention may comprise recommending the subject for a secondary clinical test to confirm a diagnosis of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

The feature sets (e.g., comprising quantitative measures of a panel of lupus condition-associated or interferon-associated genomic loci) may be analyzed and assessed (e.g., using a trained algorithm comprising one or more classifiers) over a duration of time to monitor a patient (e.g., subject who has a condition or who is being treated for a condition). In such cases, the feature sets of the patient may change during the course of treatment. For example, the quantitative measures of the feature sets of a patient with decreasing risk of the condition due to an effective treatment may shift toward the profile or distribution of a healthy subject (e.g., a subject without the condition). Conversely, for example, the quantitative measures of the feature sets of a patient with increasing risk of the condition due to an ineffective treatment may shift toward the profile or distribution of a subject with higher risk of the condition or a more advanced stage or severity of the condition.

The condition of the subject may be monitored by monitoring a course of treatment for treating the condition of the subject. The monitoring may comprise assessing the condition of the subject at two or more time points. The assessing may be based at least on the feature sets (e.g., quantitative measures of a panel of lupus condition-associated or interferon-associated genomic loci) determined at each of the two or more time points.

In some embodiments, a difference in the feature sets (e.g., quantitative measures of a panel of lupus condition-associated or interferon-associated genomic loci) determined between the two or more time points may be indicative of one or more clinical indications, such as (i) a diagnosis of the condition of the subject, (ii) a prognosis of the condition of the subject, (iii) an increased risk of the condition of the subject, (iv) a decreased risk of the condition of the subject, (v) an efficacy of the course of treatment for treating the condition of the subject, and (vi) a non-efficacy of the course of treatment for treating the condition of the subject.

In some embodiments, a difference in the feature sets (e.g., quantitative measures of a panel of lupus condition-associated or interferon-associated genomic loci) determined between the two or more time points may be indicative of a diagnosis of the condition of the subject. For example, if the condition was not detected in the subject at an earlier time point but was detected in the subject at a later time point, then the difference is indicative of a diagnosis of the condition of the subject. A clinical action or decision may be made based on this indication of diagnosis of the condition of the subject, such as, for example, prescribing a new therapeutic intervention for the subject. The clinical action or decision may comprise recommending the subject for a secondary clinical test to confirm the diagnosis of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of lupus condition-associated or interferon-associated genomic loci) determined between the two or more time points may be indicative of a prognosis of the condition of the subject.

In some embodiments, a difference in the feature sets (e.g., quantitative measures of a panel of lupus condition-associated or interferon-associated genomic loci) determined between the two or more time points may be indicative of the subject having an increased risk of the condition. For example, if the condition was detected in the subject both at an earlier time point and at a later time point, and if the difference is a negative difference (e.g., the quantitative measures of a panel of lupus condition-associated or interferon-associated genomic loci increased from the earlier time point to the later time point), then the difference may be indicative of the subject having an increased risk of the condition. A clinical action or decision may be made based on this indication of the increased risk of the condition, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of lupus condition-associated or interferon-associated genomic loci) determined between the two or more time points may be indicative of the subject having a decreased risk of the condition. For example, if the condition was detected in the subject both at an earlier time point and at a later time point, and if the difference is a positive difference (e.g., the quantitative measures of a panel of lupus condition-associated or interferon-associated genomic loci decreased from the earlier time point to the later time point), then the difference may be indicative of the subject having a decreased risk of the condition. A clinical action or decision may be made based on this indication of the decreased risk of the condition (e.g., continuing or ending a current therapeutic intervention) for the subject. The clinical action or decision may comprise recommending the subject for a secondary clinical test to confirm the decreased risk of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of lupus condition-associated or interferon-associated genomic loci) determined between the two or more time points may be indicative of an efficacy of the course of treatment for treating the condition of the subject. For example, if the condition was detected in the subject at an earlier time point but was not detected in the subject at a later time point, then the difference may be indicative of an efficacy of the course of treatment for treating the condition of the subject. A clinical action or decision may be made based on this indication of the efficacy of the course of treatment for treating the condition of the subject, e.g., continuing or ending a current therapeutic intervention for the subject. The clinical action or decision may comprise recommending the subject for a secondary clinical test to confirm the efficacy of the course of treatment for treating the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of lupus condition-associated or interferon-associated genomic loci) determined between the two or more time points may be indicative of a non-efficacy of the course of treatment for treating the condition of the subject. For example, if the condition was detected in the subject both at an earlier time point and at a later time point, and if the difference is a negative or zero difference (e.g., the quantitative measures of a panel of lupus condition-associated or interferon-associated genomic loci increased or remained at a constant level from the earlier time point to the later time point), and if an efficacious treatment was indicated at an earlier time point, then the difference may be indicative of a non-efficacy of the course of treatment for treating the condition of the subject. A clinical action or decision may be made based on this indication of the non-efficacy of the course of treatment for treating the condition of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In various embodiments, machine learning methods are applied to distinguish samples in a population of samples. In one embodiment, machine learning methods are applied to distinguish samples between healthy and lupus (e.g., SLE or DLE) samples.

Kits

The present disclosure provides kits for identifying or monitoring a disease or disorder (e.g., a lupus condition) of a subject. A kit may comprise probes for identifying a quantitative measure (e.g., indicative of a presence, absence, or relative amount) of sequences at each of a panel of lupus condition-associated or interferon-associated genomic loci in a sample of the subject. A quantitative measure (e.g., indicative of a presence, absence, or relative amount) of sequences at each of a panel of lupus condition-associated or interferon-associated genomic loci in the sample may be indicative of the disease or disorder (e.g., a lupus condition) of the subject. The probes may be selective for the sequences at the panel of lupus condition-associated or interferon-associated genomic loci in the sample. A kit may comprise instructions for using the probes to process the sample to generate datasets indicative of a quantitative measure (e.g., indicative of a presence, absence, or relative amount) of sequences at each of the panel of lupus condition-associated or interferon-associated genomic loci in a sample of the subject.

The probes in the kit may be selective for the sequences at the panel of lupus condition-associated or interferon-associated genomic loci in the sample. The probes in the kit may be configured to selectively enrich nucleic acid (e.g., RNA or DNA) molecules corresponding to the panel of lupus condition-associated or interferon-associated genomic loci. The probes in the kit may be nucleic acid primers. The probes in the kit may have sequence complementarity with nucleic acid sequences from one or more of the panel of lupus condition-associated or interferon-associated genomic loci. The panel of lupus condition-associated or interferon-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more distinct lupus condition-associated or interferon-associated genomic loci.

The instructions in the kit may comprise instructions to assay the sample using the probes that are selective for the sequences at the panel of lupus condition-associated or interferon-associated genomic loci in the cell-free biological sample. These probes may be nucleic acid molecules (e.g., RNA or DNA) having sequence complementarity with nucleic acid sequences (e.g., RNA or DNA) from one or more of the plurality of panel of lupus condition-associated or interferon-associated genomic loci. These nucleic acid molecules may be primers or enrichment sequences. The instructions to assay the cell-free biological sample may comprise introductions to perform array hybridization, polymerase chain reaction (PCR), or nucleic acid sequencing (e.g., DNA sequencing or RNA sequencing) to process the sample to generate datasets indicative of a quantitative measure (e.g., indicative of a presence, absence, or relative amount) of sequences at each of the panel of lupus condition-associated or interferon-associated genomic loci in the sample. A quantitative measure (e.g., indicative of a presence, absence, or relative amount) of sequences at each of a panel of lupus condition-associated or interferon-associated genomic loci in the sample may be indicative of a disease or disorder (e.g., a lupus condition).

The instructions in the kit may comprise instructions to measure and interpret assay readouts, which may be quantified at one or more of the panel of lupus condition-associated or interferon-associated genomic loci to generate the datasets indicative of a quantitative measure (e.g., indicative of a presence, absence, or relative amount) of sequences at each of the panel of lupus condition-associated or interferon-associated genomic loci in the sample. For example, quantification of array hybridization or polymerase chain reaction (PCR) corresponding to the panel of lupus condition-associated or interferon-associated genomic loci may generate the datasets indicative of a quantitative measure (e.g., indicative of a presence, absence, or relative amount) of sequences at each of the panel of lupus condition-associated or interferon-associated genomic loci in the sample. Assay readouts may comprise quantitative PCR (qPCR) values, digital PCR (dPCR) values, digital droplet PCR (ddPCR) values, fluorescence values, etc., or normalized values thereof.

### Low-Density Granulocyte (LDG) Profiling of Lupus Conditions

Systemic lupus erythematosus (SLE) is an autoimmune disease characterized by the presence of low-density granulocytes (LDGs) with a heightened capacity for spontaneous NETosis, but the contribution of LDGs to SLE pathogenesis may remain unclear. Systems and methods of the present disclosure may characterize LDGs in human SLE by characterizing gene expression profiles derived from isolated LDGs by weighted gene coexpression network analysis (WGCNA). A multiple-gene module (e.g., a 92-gene module) may be identified in this manner. The LDG gene signature may be enriched in genes related to neutrophil degranulation and cell cycle regulation. This signature may be assessed in gene expression datasets from two large-scale SLE clinical trials to study associations between LDG enrichment, SLE manifestations, and treatment regimens. LDG enrichment in the blood may be found to be associated with corticosteroid treatment as well as anti-dsDNA, low serum complement, renal manifestations, and vasculitis, but the latter two of these associations may be dependent on concomitant corticosteroid treatment. In addition, LDG enrichment may be found to be associated with enrichment of gene signatures induced by type I interferon (IFN) and tumor necrosis factor (TNF) irrespective of corticosteroid treatment. Notably, LDG enrichment may not be found in numerous tissues affected by SLE. Comparison with relevant reference datasets may indicate that LDG enrichment is likely reflective of increased granulopoiesis in the bone marrow and not peripheral neutrophil activation. The results obtained using systems and methods of the present disclosure may uncover important determinants of the appearance of LDGs in SLE and emphasize the likely role of LDGs in specific aspects of lupus pathogenesis.

SLE is an autoimmune disease characterized by autoreactive B cell hyperactivity, autoantibody generation, and the presence of a type I IFN gene expression signature. SLE patients may also manifest an increased population of low-density granulocytes (LDGs) in the peripheral blood that remains in the peripheral blood mononuclear cell (PBMC) fraction after Ficoll density gradient separation rather than sedimenting with normal-density granulocytes. LDGs may appear in the circulation of subjects with a number of diseases, including rheumatoid arthritis, HIV infection, cancer, tuberculosis, and Plasmodium vivax infection. Although the presence of LDGs in these conditions may tend to be associated with more severe disease, the physiologic effects of this population may be mediated by diverse pro-inflammatory and anti-inflammatory mechanisms. For example, LDGs may contribute to rheumatoid arthritis pathogenesis by exposing immunogenic citrullinated histones, whereas LDGs in HIV infection may aggravate disease by inhibiting CD4+ T cells via arginase 1.

In SLE, LDGs have been described as a pro-inflammatory subset of neutrophils with an enhanced capacity to release neutrophil extracellular traps (NETs) compared with autologous SLE neutrophils and healthy control (HC) neutrophils through a process called NETosis. During this process, neutrophils expel chromatin, antimicrobial agents, and immunostimulatory molecules into the extracellular space to trap and kill bacteria, but this process can also induce tissue damage. LDGs expose dsDNA, oxidized mitochondrial DNA, LL-37, elastase, and IL-17, among other molecules, during NETosis, and increased NETosis by LDGs may be an important source of immunostimulatory molecules and autoantigens involved in the pathogenesis of SLE.

The presence of LDGs in pediatric SLE patients may be associated with increased lupus activity as measured by the SLE Disease Activity Index (SLEDAI). LDGs have also been implicated in skin involvement and vascular damage in SLE, and netting neutrophils have been described in the glomeruli and skin of lupus patients, although it may remain unclear whether the infiltrating cells were LDGs or normal-density neutrophils.

Based on nuclear morphology and surface marker expression, LDGs have been hypothesized to be immature neutrophil precursors released from the bone marrow, perhaps related to stimulation by colony stimulating factor (CSF), such as granulocyte CSF (G-CSF) or granulocyte/macrophage CSF (GM-CSF). However, the specific origin of LDGs in SLE and, more importantly, the mechanisms by which they contribute to organ involvement and/or disease activity may remain unclear. To gain more insight into LDGs in SLE, systems and methods of the present disclosure may employ a large-scale bioinformatics approach that combines gene expression data and clinical measurements. Using systems and methods of the present disclosure, a transcriptomic signature may be generated that characterizes LDGs in SLE, to determine whether this signature can be detected in the blood and tissue of SLE patients, and to characterize the relationship between this signature and SLE disease manifestations.

The present disclosure provides systems and methods to perform genomic identification of low-density granulocytes (LDGs) and analysis of their role in the pathogenesis of systemic lupus erythematosus (SLE). Analysis of LDGs, SLE neutrophils, and HC neutrophils may reveal hundreds of genes significantly differentially expressed by LDGs and initially identify granulopoietic and proliferative signatures as potentially descriptive of LDGs. Given that circulating neutrophils do not express granulopoietic genes and that SLE neutrophils did not differentially express any genes relative to HC neutrophils, it has been posited that the detection of these signatures in SLE blood may be attributed to LDGs. However, the DE approach may be challenged by contamination from platelets and lymphocytes. LDGs may be isolated from PBMC by negative selection, using a mixture of biotinylated antibodies (Abs) to human cluster of differentiation (CD) molecules; HC and SLE neutrophils may be isolated by dextran sedimentation of red blood cell (RBC) pellets. Although the purity of LDG and neutrophil isolates may be high, the low baseline levels of transcription in neutrophils may allow even small amounts of contamination to affect microarray results strongly, so further refinement may be needed to extract a robust LDG gene expression signature.

The coexpression-based unsupervised clustering method of WGCNA may be able to dissect the gene expression landscape down into several modules of genes that separate LDG samples and neutrophil samples. One of these modules may capture what may seem to be a pattern of lymphocyte contamination in the original expression data, and another set of modules, which may be merged to form module A, may contain many of the platelet genes identified in the original DE analysis. Functional analysis may be performed to narrow the WGCNA modules down to one final module of genes, which may contain neutrophil granule genes and cell cycle regulation genes. The presence of granule genes may indicate that the module is neutrophil lineage-specific, whereas the presence of cell cycle genes after coexpression network construction may suggest that the cell cycle signature is likely descriptive of LDGs and not an artifact of the isolation protocol. The combination of neutrophil lineage-specific granule genes along with cell cycle genes may appear to identify the unique signature of LDGs. This module of genes may be strongly coexpressed in SLE blood expression data but not in lupus-affected tissue, including lupus nephritis (LN) glomerulus, LN tubulointerstitium (TI), lupus skin, and synovium. This result may indicate that the LDG gene expression signature can be recovered from blood but not from tissue. Although netting neutrophils have been described in SLE-affected glomerulus and skin, the current results may suggest that infiltrating neutrophils are either normal-density neutrophils or LDGs with an altered transcriptional program. More studies may be performed to investigate further, as LDGs may not differentially express any homing receptors or activation markers associated with the ability to infiltrate tissues.

It may be initially surprising not to find transcriptional evidence for LDGs in SLE-affected kidneys or a strong association between LDG enrichment and renal involvement, as a similar group of neutrophil genes may be found to be enriched in the blood of LN patients compared with lupus patients without nephritis. A claim of an association with neutrophils may be based on a gene module, M5.15, derived from modular repertoire analysis and consisting of 24 neutrophilspecific genes, 14 of which overlap with LDG module B. Notably, both LDG module B and M5.15 may contain a core signature of 10 granulopoiesis-related genes that are not part of an endotoxemia-induced neutrophil activation signature (AZU1, CAMP, CEACAM6, CEACAM8, CTSG, DEFA4, ELANE, LTF, MPO, and MS4A3). This may suggest that module M5.15 may not describe neutrophil activation but rather the presence of LDGs. A limitation may be that the presence of rapidly progressive or severe renal disease excludes patients from the ILLUMINATE trials, so an association of active renal disease with enrichment of LDGs may be missed. Therefore, enrichment of LDG genes may not yet be ruled out as a potential biomarker for LN. It may be notable that an association between the LDG signature in the blood and renal involvement in the current study may only be noted in those patients receiving corticosteroids. Whether the usage of corticosteroids is a surrogate for disease activity in this circumstance may not be further delineated, but it may suggest that LDG module B and similar signatures may be of diagnostic use to identify those with LN only in the subset of patients taking corticosteroids.

By taking a large-scale transcriptomics approach to quantify the enrichment of the LDG signature in SLE blood gene expression data, it may be possible to draw associations between LDG enrichment and clinical measurements of disease manifestation by studying both relative enrichment scores and binary LDG enrichment. LDG enrichment may be associated with increased disease activity estimated by SLEDAI, decreased complement levels, and the presence of anti-dsDNA, suggesting that LDGs can act as markers of serological disease activity. Because complement levels and anti-dsDNA are components of the SLEDAI score, it is possible that these measurements account for the association with increased SLEDAI, as the associations with anti-dsDNA and low complement may be stronger than the association with SLEDAI score.

The association between corticosteroid use and LDG enrichment may be notable. Patients taking corticosteroids may have significantly higher LDG enrichment than those not taking corticosteroids, and some disease manifestations may only be associated with LDG enrichment in patients taking corticosteroids. It may be unknown at this time whether increased LDG enrichment among patients using corticosteroids is related to increased granulopoiesis in the bone marrow or demargination of LDGs from the endothelium. Other studies may suggest that the major effect of corticosteroids on distribution of cells of the neutrophil lineage relates to demargination, although this may not be known for LDGs. However, the findings may suggest that at least one component of the appearance of increased LDGs in the blood of lupus patients relates to corticosteroid-induced demargination. It may be suggested that LDGs play a role in SLE vascular pathology. It may be possible, therefore, that LDGs home to the endothelium and contribute to local vascular inflammation. In this situation, corticosteroid-induced demargination may be therapeutically useful by dissociating LDGs from the vascular endothelium. The relationship between circulating LDGs and vascular pathology may be complex, and a better understanding of whether corticosteroid use stimulates LDG production or alternatively causes demargination of LDGs may therefore be essential to resolve this conundrum.

The presence of LDG-specific genes in bone marrow myeloid precursors may support the hypothesis that LDGs are related to early neutrophil precursors (PM or MY) released from the bone marrow in response to cytokine challenge. Other studies may suggest that there may be two populations of LDGs in tumor-bearing mice and humans: one originating from the bone marrow and the second from peripheral neutrophils as a result of TGF-b stimulation. Similarly, present results may indicate that LDGs overexpress CD66b (CEACAM8), but no evidence of upregulation of the TGF-b signaling pathway may be found. These results may be most consistent with the conclusion that the LDGs expanded in SLE are most similar to early neutrophil precursors and not TGF-b-stimulated mature neutrophils. Taken together with the strong association between LDG enrichment and TNF response, these results may suggest that another component of the increased appearance of LDGs in the blood of lupus patients may relate to their enhanced release from the bone marrow as a result of chronic TNF-induced production of G-CSF. The associations between LDG enrichment and both low complement levels (indicative of complement consumption, presumably owing to the presence of immune complexes) and a TNF response may suggest that LDGs are part of an acute phase-like response in SLE. Autoantibodies to dsDNA may be found to be present in ˜73% of patients with positive LDG enrichment, and an IFN signature may be seen in 98% of patients with LDGs. These results may be consistent with a role for autoantibodies and/or autoantibody containing immune complexes in the appearance of LDGs in the circulation either directly or through the induction of cytokines, such as type I IFN or TNF. Alternatively, LDGs may play a role in the induction of autoantibodies, as LDG NETS may be autoantigenic and interferogenic.

Systems and methods of the present disclosure may comprise analysis of bulk RNA from blood and various lupus-affected tissues and, as a result, may not explore the possible heterogeneity of LDGs at the single-cell level. Single-cell transcriptomic studies of LDGs in SLE may be performed to further elucidate the characteristics of this cell population and whether a related population is present in lupus-affected tissues. A deeper understanding of any subtypes of LDGs and how they differ in composition among SLE patients may offer unique insights into disease processes and therapeutic options for patients with circulating LDGs.

The current results may suggest that LDGs are not directly involved in inflammation in SLE-affected organs, but they may act as biomarkers of processes that can in parallel result in tissue damage or vascular damage. As LDGs are associated with anti-dsDNA, low serum complement, and the presence of an IGS, they may indirectly lead to increasingly severe disease in afflicted patients. However, the possibility that factors such as treatment regimens may contribute to the presence of LDGs may not be dismissed because of their association with increased disease activity, highlighting the complexity of the association of LDGs with disease manifestations in SLE. Further studies of LDGs may be performed to help understand the links between corticosteroid treatment, LDG enrichment, and SLE pathogenesis.

In one aspect, the present disclosure provides a method for identifying a lupus condition of a subject, comprising: (a) assaying a biological sample of the subject to generate a dataset comprising gene expression data; (b) processing the dataset at each of a plurality of genes to determine quantitative measures of each of the plurality of genes, wherein the plurality of genes comprises low-density granulocyte (LDG)-associated genes, thereby producing an LDG signature of the biological sample of the subject; (c) comparing the LDG signature with one or more reference LDG signatures, wherein the comparing comprises, for at least one of the plurality of genes, comparing the quantitative measure of the gene of the LDG signature with corresponding quantitative measures of the gene of the one or more reference LDG signatures; (d) based at least in part on the comparison in (c), identifying the lupus condition of the subject.

In some embodiments, the lupus condition is selected from the group consisting of: systemic lupus erythematosus (SLE), discoid lupus erythematosus (DLE), and lupus nephritis (LN). In some embodiments, the biological sample is selected from the group consisting of: a whole blood (WB) sample, a PBMC sample, a tissue sample, and a cell sample. In some embodiments, the tissue sample is selected from the group consisting of: skin tissue, synovium tissue, kidney tissue, and bone marrow tissue. In some embodiments, the kidney tissue is selected from the group consisting of: glomerulus (Glom) and tubulointerstitium (TI). In some embodiments, the cell sample is selected from the group consisting of: myelocytes (MY), promyelocytes (PM), polymorphonuclear neutrophils (PMN), and peripheral blood mononuclear cells (PBMC).

In some embodiments, the method further comprises enriching or purifying a whole blood sample of the subject to obtain the cell sample. In some embodiments, assaying the biological sample comprises (i) using a microarray to generate the dataset comprising the gene expression data, (ii) sequencing the biological sample to generate the dataset comprising the gene expression data, or (iii) performing quantitative polymerase chain reaction (qPCR) of the biological sample to generate the dataset comprising the gene expression data.

In some embodiments, the plurality of genes comprises LDG-associated genes selected from the genes listed in Table 33. In some embodiments, the plurality of genes comprises LDG-associated genes selected from the genes listed in Table 34. In some embodiments, the plurality of genes comprises LDG-associated genes selected from the genes listed in Table 42A or Table 42B. In some embodiments, the plurality of genes comprises LDG-associated genes selected from the genes listed in Table 43A-43C. In some embodiments, the plurality of genes comprises LDG-associated genes selected from the genes listed in Table 44A. In some embodiments, the plurality of genes comprises LDG-associated genes selected from the genes listed in Table 45A or Table 45B.

In some embodiments, the quantitative measures of each of the plurality of genes comprise enrichment scores of each of the plurality of genes. In some embodiments, the enrichment scores of each of the plurality of genes comprise gene set variation analysis (GSVA) enrichment scores of each of the plurality of genes. In some embodiments, (c) further comprises, for the at least one of the plurality of genes, determining a difference between the quantitative measure of the gene of the LDG signature with the corresponding quantitative measures of the gene of the one or more reference LDG signatures. In some embodiments, (d) further comprises identifying the lupus condition of the subject when the difference satisfies a pre-determined criterion.

In some embodiments, (c) further comprises, for the at least one of the plurality of genes, determining a Z-score of the quantitative measure of the gene of the LDG signature relative to the corresponding quantitative measures of the gene of the one or more reference LDG signatures. In some embodiments, (d) further comprises identifying the lupus condition of the subject when the Z-score satisfies a pre-determined criterion. In some embodiments, (d) further comprises identifying the lupus condition of the subject when the Z-score is at least 2, and identifying an absence of the lupus condition of the subject when the Z-score is less than 2.

In some embodiments, the method further comprises identifying the lupus condition of the subject at a sensitivity of at least about 70%. In some embodiments, the method further comprises identifying the lupus condition of the subject at a sensitivity of at least about 80%. In some embodiments, the method further comprises identifying the lupus condition of the subject at a sensitivity of at least about 90%.

In some embodiments, the method further comprises identifying the lupus condition of the subject at a specificity of at least about 70%. In some embodiments, the method further comprises identifying the lupus condition of the subject at a specificity of at least about 80%. In some embodiments, the method further comprises identifying the lupus condition of the subject at a specificity of at least about 90%.

In some embodiments, the method further comprises identifying the lupus condition of the subject at a positive predictive value (PPV) of at least about 70%. In some embodiments, the method further comprises identifying the lupus condition of the subject at a positive predictive value (PPV) of at least about 80%. In some embodiments, the method further comprises identifying the lupus condition of the subject at a positive predictive value (PPV) of at least about 90%.

In some embodiments, the method further comprises identifying the lupus condition of the subject at a negative predictive value (NPV) of at least about 70%. In some embodiments, the method further comprises identifying the lupus condition of the subject at a negative predictive value (NPV) of at least about 80%. In some embodiments, the method further comprises identifying the lupus condition of the subject at a negative predictive value (NPV) of at least about 90%.

In some embodiments, the method further comprises identifying the lupus condition of the subject with an Area Under Curve (AUC) of at least about 0.70. In some embodiments, the method further comprises identifying the lupus condition of the subject with an Area Under Curve (AUC) of at least about 0.80. In some embodiments, the method further comprises identifying the lupus condition of the subject with an Area Under Curve (AUC) of at least about 0.90.

In some embodiments, (d) further comprises identifying the lupus condition of the subject based at least in part on a SLEDAI score of the subject. In some embodiments, the subject is asymptomatic for one or more lupus conditions selected from the group consisting of: systemic lupus erythematosus (SLE), discoid lupus erythematosus (DLE), and lupus nephritis (LN).

In some embodiments, the method further comprises applying a trained algorithm to the LDG signature to identify the lupus condition of the subject. In some embodiments, the trained algorithm is trained using a first set of independent training samples associated with a presence of the lupus condition and a second set of independent training samples associated with an absence of the lupus condition. In some embodiments, the method further comprises using the trained algorithm to process a set of clinical health data of the subject to identify the lupus condition. In some embodiments, the trained algorithm comprises a supervised machine learning algorithm. In some embodiments, the supervised machine learning algorithm comprises a deep learning algorithm, a support vector machine (SVM), a neural network, or a Random Forest.

In some embodiments, (a) comprises (i) subjecting the biological sample to conditions that are sufficient to isolate, enrich, or extract a plurality of nucleic acid molecules; and (ii) analyzing the plurality of nucleic acid molecules to generate the dataset comprising the gene expression data.

In some embodiments, the method further comprises using probes configured to selectively enrich the plurality of nucleic acid molecules corresponding to a panel of one or more genomic loci. In some embodiments, the probes are nucleic acid primers. In some embodiments, the probes have sequence complementarity with nucleic acid sequences of the panel of the one or more genomic loci. In some embodiments, the panel of the one or more genomic loci comprises genomic loci corresponding to the plurality of genes. In some embodiments, the panel of said one or more genomic loci comprises at least 5 distinct genomic loci. In some embodiments, the panel of said one or more genomic loci comprises at least 10 distinct genomic loci.

In some embodiments, the method further comprises (e) assaying a second biological sample of the subject to generate a second dataset comprising gene expression data; (f) processing the second dataset at each of the plurality of genes to determine second quantitative measures of each of the plurality of genes, thereby producing a second LDG signature of the second biological sample of the subject; (g) comparing the second LDG signature with one or more reference LDG signatures, wherein the comparing comprises, for at least one of the plurality of genes, comparing the quantitative measure of the gene of the second LDG signature with corresponding quantitative measures of the gene of the one or more reference LDG signatures; and (h) based at least in part on the comparison in (g), identifying the lupus condition of the subject.

In some embodiments, the biological sample and the second biological sample comprise two different sample types selected from the group consisting of: a whole blood (WB) sample, a PBMC sample, a skin tissue sample, a synovium tissue sample, a kidney tissue sample comprising glomerulus (Glom), a kidney tissue sample comprising tubulointerstitium (TI), a bone marrow tissue, a myelocyte (MY) cell sample, a promyelocyte (PM) cell sample, and a polymorphonuclear neutrophils (PMN) sample.

In some embodiments, the method further comprises determining a likelihood of the identification of the lupus condition of the subject. In some embodiments, the method further comprises providing a therapeutic intervention for the lupus condition of the subject.

In some embodiments, the method further comprises monitoring the lupus condition of the subject, wherein the monitoring comprises assessing the lupus condition of the subject at a plurality of time points, wherein the assessing is based at least on the lupus condition identified in (d) at each of the plurality of time points.

In some embodiments, a difference in the assessment of the lupus condition of the subject among the plurality of time points is indicative of one or more clinical indications selected from the group consisting of: (i) a diagnosis of the lupus condition of the subject, (ii) a prognosis of the lupus condition of the subject, and (iii) an efficacy or non-efficacy of a course of treatment for treating the lupus condition of the subject.

In some embodiments, the one or more reference LDG signatures are generated by: assaying a biological sample of one or more patients having one or more disease symptoms or being treated with one or more drugs to generate a reference dataset comprising gene expression data; and processing the reference dataset at each of the plurality of genes to determine quantitative measures of each of the plurality of genes.

In some embodiments, the one or more disease symptoms are selected from the group consisting of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance.

In some embodiments, the one or more drugs are selected from the group consisting of: antimalarials, corticosteroids, immunosuppressants, and nonsteroidal anti-inflammatory drugs (NSAIDs).

In another aspect, the present disclosure provides a computer system for identifying a lupus condition of a subject, comprising: a database that is configured to store a dataset comprising gene expression data, wherein the gene expression data is obtained by assaying a biological sample of the subject; and one or more computer processors operatively coupled to the database, wherein the one or more computer processors are individually or collectively programmed to: (i) process the dataset at each of a plurality of genes to determine quantitative measures of each of the plurality of genes, wherein the plurality of genes comprises low-density granulocyte (LDG)-associated genes, thereby producing an LDG signature of the biological sample of the subject; (ii) compare the LDG signature with one or more reference LDG signatures, wherein the comparing comprises, for at least one of the plurality of genes, comparing the quantitative measure of the gene of the LDG signature with corresponding quantitative measures of the gene of the one or more reference LDG signatures; and (iii) based at least in part on the comparison in (ii), identify the lupus condition of the subject.

In some embodiments, the computer system further comprises an electronic display operatively coupled to the one or more computer processors, wherein the electronic display comprises a graphical user interface that is configured to display the report.

In another aspect, the present disclosure provides a non-transitory computer readable medium comprising machine-executable code that, upon execution by one or more computer processors, implements a method for identifying a lupus condition of a subject, the method comprising: (a) assaying a biological sample of the subject to generate a dataset comprising gene expression data; (b) processing the dataset at each of a plurality of genes to determine quantitative measures of each of the plurality of genes, wherein the plurality of genes comprises low-density granulocyte (LDG)-associated genes, thereby producing an LDG signature of the biological sample of the subject; (c) comparing the LDG signature with one or more reference LDG signatures, wherein the comparing comprises, for at least one of the plurality of genes, comparing the quantitative measure of the gene of the LDG signature with corresponding quantitative measures of the gene of the one or more reference LDG signatures; (d) based at least in part on the comparison in (c), identifying the lupus condition of the subject.

To obtain a blood sample, various techniques may be used, e.g., a syringe or other vacuum suction device. A blood sample can be optionally pre-treated or processed prior to use. A sample, such as a blood sample, may be analyzed under any of the methods and systems herein within 4 weeks, 2 weeks, 1 week, 6 days, 5 days, 4 days, 3 days, 2 days, 1 day, 12 hr, 6 hr, 3 hr, 2 hr, or 1 hr from the time the sample is obtained, or longer if frozen. When obtaining a sample from a subject (e.g., blood sample), the amount can vary depending upon subject size and the condition being screened. In some embodiments, at least 10 mL, 5 mL, 1 mL, 0.5 mL, 250, 200, 150, 100, 50, 40, 30, 20, 10, 9, 8, 7, 6, 5, 4, 3, 2, or 1 μL of a sample is obtained. In some embodiments, 1-50, 2-40, 3-30, or 4-20 μL of sample is obtained. In some embodiments, more than 5, 10, 15, 20, 25, 30, 35, 40, 45, 50, 55, 60, 65, 70, 75, 80, 85, 90, 95 or 100 μL of a sample is obtained.

The sample may be taken before and/or after treatment of a subject with a disease or disorder. Samples may be obtained from a subject during a treatment or a treatment regime. Multiple samples may be obtained from a subject to monitor the effects of the treatment over time. The sample may be taken from a subject known or suspected of having a disease or disorder for which a definitive positive or negative diagnosis is not available via clinical tests. The sample may be taken from a subject suspected of having a disease or disorder. The sample may be taken from a subject experiencing unexplained symptoms, such as fatigue, nausea, weight loss, aches and pains, weakness, or bleeding. The sample may be taken from a subject having explained symptoms. The sample may be taken from a subject at risk of developing a disease or disorder due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

In some embodiments, a sample can be taken at a first time point and assayed, and then another sample can be taken at a subsequent time point and assayed. Such methods can be used, for example, for longitudinal monitoring purposes to track the development or progression of a disease. In some embodiments, the progression of a disease can be tracked before treatment, after treatment, or during the course of treatment, to determine the treatment's effectiveness. For example, a method as described herein can be performed on a subject prior to, and after, treatment with a lupus condition therapy to measure the disease's progression or regression in response to the lupus condition therapy.

After obtaining a sample from the subject, the sample may be processed to generate datasets indicative of a disease or disorder of the subject. For example, a presence, absence, or quantitative assessment of nucleic acid molecules of the sample at a panel of lupus condition-associated or LDG-associated genomic loci or may be indicative of a lupus condition of the subject. Processing the sample obtained from the subject may comprise (i) subjecting the sample to conditions that are sufficient to isolate, enrich, or extract a plurality of nucleic acid molecules, and (ii) assaying the plurality of nucleic acid molecules to generate the dataset (e.g., microarray data, nucleic acid sequences, or quantitative polymerase chain reaction (qPCR) data). Methods of assaying may include any assay known in the art or described in the literature, for example, a microarray assay, a sequencing assay (e.g., DNA sequencing, RNA sequencing, or RNA-Seq), or a quantitative polymerase chain reaction (qPCR) assay.

In some embodiments, a plurality of nucleic acid molecules is extracted from the sample and subjected to sequencing to generate a plurality of sequencing reads. The nucleic acid molecules may comprise ribonucleic acid (RNA) or deoxyribonucleic acid (DNA). The extraction method may extract all RNA or DNA molecules from a sample. Alternatively, the extraction method may selectively extract a portion of RNA or DNA molecules from a sample. Extracted RNA molecules from a sample may be converted to cDNA molecules by reverse transcription (RT).

The sample may be processed without any nucleic acid extraction. For example, the disease or disorder may be identified or monitored in the subject by using probes configured to selectively enrich nucleic acid (e.g., RNA or DNA) molecules corresponding to a panel of lupus condition-associated or LDG-associated genomic loci. The probes may be nucleic acid primers. The probes may have sequence complementarity with nucleic acid sequences from one or more of the panel of lupus condition-associated or LDG-associated genomic loci. The panel of lupus condition-associated or LDG-associated genomic loci may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more lupus condition-associated or LDG-associated genomic loci.

The probes may be nucleic acid molecules (e.g., RNA or DNA) having sequence complementarity with nucleic acid sequences (e.g., RNA or DNA) of one or more genomic loci (e.g., lupus condition-associated or LDG-associated genomic loci). These nucleic acid molecules may be primers or enrichment sequences. The assaying of the sample using probes that are selective for the one or more genomic loci (e.g., lupus condition-associated or LDG-associated genomic loci) may comprise use of array hybridization, polymerase chain reaction (PCR), or nucleic acid sequencing (e.g., RNA sequencing or DNA sequencing, such as RNA-Seq).

The assay readouts may be quantified at one or more genomic loci (e.g., lupus condition-associated or LDG-associated genomic loci) to generate the data indicative of the disease or disorder. For example, quantification of array hybridization or polymerase chain reaction (PCR) corresponding to a plurality of genomic loci (e.g., lupus condition-associated or LDG-associated genomic loci) may generate data indicative of the disease or disorder. Assay readouts may comprise quantitative PCR (qPCR) values, digital PCR (dPCR) values, digital droplet PCR (ddPCR) values, fluorescence values, etc., or normalized values thereof.

Methods

Gene expression data may be compiled from SLE patients as follows. Data are derived from publicly available datasets on Gene Expression Omnibus (<https://www.ncbi.nlm.nih.gov/geo/>) and collaborators. Raw data sources are as follows: LDGs (GSE26975 [9 healthy control (HC) neutrophils, 10 SLE neutrophils, and 10 SLE LDGs]), PBMCs (GSE50772 [20 HC and 59 SLE], GSE81622 [25 HC and 30 SLE], FDABMC3 [6 HC and 43 SLE]), whole blood (WB) (GSE49454 [10 HC and 49 SLE], GSE88884 [17 HC and 1612 SLE]), kidney glomerulus and tubulointerstitium (TI) (GSE32591 [14 HC and 30 lupus nephritis (LN)]), skin (GSE52471 [3 HC and 7 discoid lupus erythematosus (DLE)], GSE72535 [8 HC and 9 DLE]), synovium (GSE36700 [4 osteoarthritis (OA) and 4 SLE]), and bone marrow myeloid lineage cells (GSE19556 [6 promyelocytes (PM), 6 myelocytes (MY), 6 bone marrow polymorphonuclear neutrophils (PMN), and 6 peripheral blood PMN]). Clinical data, when available, including disease activity assessed by SLEDAI, anti-dsDNA titers, and complement levels, may be included in the analysis.

Quantity control and normalization of raw data files may be performed as follows. Statistical analysis is conducted using R and relevant Bioconductor packages. Nonnormalized arrays are inspected for visual artifacts or poor RNA hybridization using Affy quality control plots. To inspect the raw data files for outliers, principal component analysis plots are generated for all cell types available for each experiment. Datasets culled of outliers are cleaned of background noise and normalized using GeneChip robust multiarray averaging, resulting in log 2 intensity values compiled into Rexpression set objects (E-sets). To increase the probability of identifying differentially expressed genes (DEGs), analysis is conducted using normalized datasets prepared using the native Affy chip definition files (CDFs), followed by custom BrainArray (BA) Entrez CDFs maintained by the University of Michigan Molecular and Behavioral Neuroscience Institute. The Affy CDFs include multiple probes per gene and almost twice as many probes as BA CDFs. Although Affy CDFs can provide the greatest amount of variance information for Bayesian fitting, the BA CDFs are used to exclude probes with known nonspecific binding and those shown by quarterly BLASTs to no longer fall within the target gene. Illumina CDFs are used for the Illumina datasets (GSE49454, GSE81622).

Differential gene expression (DE) analysis may be performed as follows. The CDF-annotated E-sets are filtered to remove probes with very low-intensity values. This reduces the E-set dimensions and the degree of multiple hypothesis testing correction, which increases the statistical significance of the differential expression (DE) probes. Probes missing gene annotation data are also discarded. GeneChip robust multiarray averaging-normalized expression values are variance corrected using local empirical Bayesian shrinkage before calculation of DE, using the ebayes function in the Bioconductor limma package. Resulting p values are adjusted for multiple hypothesis testing using the Benjamini-Hochberg correction, which results in a false discovery rate (FDR). Significant Affy and BA probes within each study are merged and filtered to retain DE probes with an FDR<0.05, which are considered statistically significant. This list is further filtered to retain only the most significant probe per gene to remove duplicate probes.

Weighted gene coexpression network analysis (WGCNA) may be performed as follows. Log 2 normalized microarray expression values are used as input to weighted gene coexpression network analysis (WGCNA) to conduct an unsupervised clustering analysis, resulting in coexpression “modules,” or groups of densely interconnected genes, which may correspond to comparably regulated biologic pathways. For each experiment, an approximately scale-free topology matrix is first calculated to encode the network strength between probes. Probes are clustered into WGCNA modules based on topology matrix distances. Resultant dendrograms of correlation networks are trimmed to isolate individual modular groups of probes, labeled using semi-random color assignments, based on a detection cut height of 1, with a merging cut height of 0.2, with the additional use of a partitioning around medoids function. Final membership of probes representing the same gene into modules is based on selection of the greatest within-module correlation with module eigengene (ME) values.

Expression profiles of genes within modules are summarized by an ME, the module's first principal component. MEs act as characteristic expression values for their respective modules and can be associated with sample traits such as cell type, cohort (HC or SLE), or serological measurements. This is done by Welch's t test. The correlation coefficient of each gene in a module with the ME (kME), a metric for module membership, is used to determine the association of individual genes with the expression of the module as a whole. The mean kME of all genes in a module is taken as a metric of overall module quality. If the genes in a module have low kMEs, it is indicative that a few highly variable genes dominate the eigengene calculation. Modules with mean kMEs close to 1 are considered to be high quality, and modules with mean kMEs close to 0 are considered to be low quality. When analyzing multiple datasets, the grand mean is the mean of the mean kMEs for each dataset.

Cytoscape and STRING may be used to create MCODE clusters as follows. STRING (v10.5) is used to score protein—protein interaction networks, which are visualized using the Cytoscape (v3.5.1) software. The clusterMaker2 (v1.1.0) plugin application is used to create MCODE clusters of the most closely related genes.

Gene Set Variation Analysis (GSVA) may be performed as follows. The gene set variation analysis (GSVA) Bioconductor package is used as a nonparametric, unsupervised method for estimating the variation of predefined gene sets in patient and control samples of microarray expression datasets. The GSVA algorithm accepts a gene expression matrix of log 2-transformed expression values and a collection of predefined gene sets as inputs. Enrichment scores are calculated nonparametrically using a Kolmogorov-Smirnov-like random walk statistic. The enrichment scores are the largest positive and negative random walk deviations from zero, respectively, for a particular sample and gene set. Individual patient gene expression sets are considered positively enriched for a given signature if they display a z-score of greater than 2 relative to controls. Individual patient gene expression sets are considered negatively enriched for a given signature if they display a z-score of less than 2 relative to controls. Analysis of GSVA scores is carried out using Fisher's exact test or Welch's unequal variances t test, where appropriate.

Other statistical analyses may be performed as follows. The p values resulting from DE analysis are adjusted by the Benjamini-Hochberg FDR correction. Analysis of parametric data is performed using a two-tailed Welch's t test. Correlation analysis of continuous variables is performed by Pearson correlation, and analysis of noncontinuous variables is performed by Spearman rank correlation. Correlations are reported as Pearson r or Spearman rho, as appropriate. Odds ratio analysis is performed by Fisher's exact test, and odds ratios are accompanied by 95% confidence intervals.

Classifiers

In some embodiments, the present disclosure provides a system, method, or kit having data analysis realized in software application, computing hardware, or both. In various embodiments, the analysis application or system includes at least a data receiving module, a data pre-processing module, a data analysis module, a data interpretation module, or a data visualization module. In one embodiment, the data receiving module can comprise computer systems that connect laboratory hardware or instrumentation with computer systems that process laboratory data. In one embodiment, the data pre-processing module can comprise hardware systems or computer software that performs operations on the data in preparation for analysis. Examples of operations that can be applied to the data in the pre-processing module include affine transformations, denoising operations, data cleaning, reformatting, or subsampling. A data analysis module, which can be specialized for analyzing genomic data from one or more genomic materials, can, for example, take assembled genomic sequences and perform probabilistic and statistical analysis to identify abnormal patterns related to a disease, pathology, state, risk, condition, or phenotype. A data interpretation module can use analysis methods, for example, drawn from statistics, mathematics, or biology, to support understanding of the relation between the identified abnormal patterns and health conditions, functional states, prognoses, or risks. A data visualization module can use methods of mathematical modeling, computer graphics, or rendering to create visual representations of data that can facilitate the understanding or interpretation of results.

Feature sets may be generated from datasets obtained using one or more assays of a biological sample, and a trained algorithm may be used to process one or more of the feature sets to identify or assess the condition (e.g., a disease or disorder, such as a lupus condition). For example, the trained algorithm may be used to apply a machine learning classifier to a plurality of lupus condition-associated or LDG-associated genomic loci that are associated with two or more classes of individuals inputted into a machine learning model, in order to classify a subject into one of the two or more classes of individuals. For example, the trained algorithm may be used to apply a machine learning classifier to a plurality of lupus condition-associated or LDG-associated genomic loci that are associated with individuals with known conditions (e.g., a disease or disorder, such as a lupus condition) and individuals not having the condition (e.g., healthy individuals, or individuals who do not have a lupus condition), in order to classify a subject as having the condition (e.g., positive test outcome) or not having the condition (e.g., negative test outcome).

The trained algorithm may be configured to identify the presence (e.g., positive test result) or absence (e.g., negative test result) of one or more conditions (e.g., a disease or disorder, such as a lupus condition) with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99%. This accuracy may be achieved for a set of at least about 25, at least about 50, at least about 100, at least about 150, at least about 200, at least about 250, at least about 300, at least about 350, at least about 400, at least about 450, at least about 500, at least about 1,000, or more than about 1,000 independent samples.

The trained algorithm may comprise a machine learning algorithm, such as a supervised machine learning algorithm. The supervised machine learning algorithm may comprise, for example, a Random Forest, a support vector machine (SVM), a neural network, or a deep learning algorithm. The trained algorithm may comprise a classification and regression tree (CART) algorithm. The trained algorithm may comprise an unsupervised machine learning algorithm.

The trained algorithm may comprise a classifier configured to accept as input a plurality of input variables or features (e.g., lupus condition-associated or LDG-associated genomic loci) and to produce or output one or more output values based on the plurality of input variables or features (e.g., lupus condition-associated or LDG-associated genomic loci). The plurality of input variables or features may comprise one or more datasets indicative of the presence (e.g., positive test result) or absence (e.g., negative test result) of one or more conditions (e.g., a disease or disorder, such as a lupus condition). For example, an input variable or feature may comprise a number of sequences corresponding to or aligning to each of the plurality of lupus condition-associated or LDG-associated genomic loci.

The plurality of input variables or features may also include clinical information of a subject, such as health data. For example, the health data of a subject may comprise one or more of: a diagnosis of one or more conditions (e.g., a disease or disorder, such as a lupus condition), a prognosis of one or more conditions (e.g., a disease or disorder, such as a lupus condition), a risk of having one or more conditions (e.g., a disease or disorder, such as a lupus condition), a treatment history of one or more conditions (e.g., a disease or disorder, such as a lupus condition), a history of previous treatment for one or more conditions (e.g., a disease or disorder, such as a lupus condition), a history of prescribed medications, a history of prescribed medical devices, age, height, weight, sex, smoking status, and one or more symptoms of the subject.

For example, the disease or disorder may comprise one or more of: systemic lupus erythematosus (SLE), discoid lupus erythematosus (DLE), and lupus nephritis (LN). As another example, the symptoms may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof. As another example, the prescribed medications or drugs may include one or more of: antimalarials, corticosteroids, immunosuppressants, and nonsteroidal anti-inflammatory drugs (NSAIDs).

The trained algorithm may comprise a classifier, such that each of the one or more output values comprises one of a fixed number of possible values (e.g., a linear classifier, a logistic regression classifier, etc.) indicating a classification of the sample by the classifier. The trained algorithm may comprise a binary classifier, such that each of the one or more output values comprises one of two values (e.g., {0, 1}, {positive, negative}, or {high-risk, low-risk}) indicating a classification of the sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the sample by the classifier.

The classifier may be configured to classify samples by assigning output values, which may comprise descriptive labels, numerical values, or a combination thereof. Some of the output values may comprise descriptive labels. Such descriptive labels may provide an identification or indication of the presence (e.g., positive test result) or absence (e.g., negative test result) of one or more conditions (e.g., a disease or disorder, such as a lupus condition) of the subject, and may comprise, for example, positive, negative, high-risk, intermediate-risk, low-risk, or indeterminate. Such descriptive labels may provide an identification of a treatment for the one or more conditions of the subject, and may comprise, for example, a therapeutic intervention, a duration of the therapeutic intervention, and/or a dosage of the therapeutic intervention suitable to treat the one or more conditions of the subject. Such descriptive labels may provide an identification of secondary clinical tests that may be appropriate to perform on the subject, and may comprise, for example, an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof. For example, such descriptive labels may provide a prognosis of the one or more conditions of the subject. As another example, such descriptive labels may provide a relative assessment of the one or more conditions of the subject. Some descriptive labels may be mapped to numerical values, for example, by mapping “positive” to 1 and “negative” to 0.

The classifier may be configured to classify samples by assigning output values that comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the one or more conditions (e.g., a disease or disorder, such as a lupus condition) of the subject. Some numerical values may be mapped to descriptive labels, for example, by mapping 1 to “positive” and 0 to “negative.”

The classifier may be configured to classify samples by assigning output values based on one or more cutoff values. For example, a binary classification of samples may assign an output value of “positive” or 1 if the sample indicates that the subject has at least a 50% probability of having one or more conditions (e.g., a disease or disorder, such as a lupus condition), thereby assigning the subject to a class of individuals receiving a positive test result. As another example, a binary classification of samples may assign an output value of “negative” or 0 if the sample indicates that the subject has less than a 50% probability of having one or more conditions (e.g., a disease or disorder), thereby assigning the subject to a class of individuals receiving a negative test result. In this case, a single cutoff value of 50% is used to classify samples into one of the two possible binary output values or classes of individuals (e.g., those receiving a positive test result and those receiving a negative test result). Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, the classifier may be configured to classify samples by assigning an output value of “positive” or 1 if the sample indicates that the subject has a probability of having one or more conditions (e.g., a disease or disorder, such as a lupus condition) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of having one or more conditions (e.g., a disease or disorder, such as a lupus condition) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classifier may be configured to classify samples by assigning an output value of “negative” or 0 if the sample indicates that the subject has a probability of having one or more conditions (e.g., a disease or disorder, such as a lupus condition) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having one or more conditions (e.g., a disease or disorder, such as a lupus condition) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classifier may be configured to classify samples by assigning an output value of “indeterminate” or 2 if the sample is not classified as “positive”, “negative”, 1, or 0. In this case, a set of two cutoff values is used to classify samples into one of the three possible output values or classes of individuals (e.g., corresponding to outcome groups of individuals having “low risk,” “intermediate risk,” and “high risk” of having one or more conditions, such as a disease or disorder). Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values or classes of individuals, where n is any positive integer.

The trained algorithm may be trained with a plurality of independent training samples. Each of the independent training samples may comprise a sample from a subject, associated datasets obtained by assaying the sample (as described elsewhere herein), and one or more known output values or classes of individuals corresponding to the sample (e.g., a clinical diagnosis, prognosis, absence, or treatment efficacy of a condition of the subject). Independent training samples may comprise samples and associated datasets and outputs obtained or derived from a plurality of different subjects. Independent training samples may comprise samples and associated datasets and outputs obtained at a plurality of different time points from the same subject (e.g., on a regular basis such as weekly, biweekly, or monthly), as part of a longitudinal monitoring of a subject before, during, and after a course of treatment for one or more conditions of the subject. Independent training samples may be associated with presence of the condition (e.g., training samples comprising samples and associated datasets and outputs obtained or derived from a plurality of subjects known to have the condition). Independent training samples may be associated with absence of the condition (e.g., training samples comprising samples and associated datasets and outputs obtained or derived from a plurality of subjects who are known to not have a previous diagnosis of the condition or who have received a negative test result for the condition).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise samples associated with presence of the condition and/or samples associated with absence of the condition. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the condition (e.g., a disease or disorder, such as a lupus condition). The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with absence of the condition (e.g., a disease or disorder, such as a lupus condition). In some embodiments, the sample is independent of samples used to train the trained algorithm.

The trained algorithm may be trained with a first number of independent training samples associated with a presence of the condition (e.g., a disease or disorder, such as a lupus condition) and a second number of independent training samples associated with an absence of the condition (e.g., a disease or disorder, such as a lupus condition). The first number of independent training samples associated with presence of the condition (e.g., a disease or disorder, such as a lupus condition) may be no more than the second number of independent training samples associated with absence of the condition (e.g., a disease or disorder, such as a lupus condition). The first number of independent training samples associated with a presence of the condition (e.g., a disease or disorder) may be equal to the second number of independent training samples associated with an absence of the condition (e.g., a disease or disorder, such as a lupus condition). The first number of independent training samples associated with a presence of the condition (e.g., a disease or disorder, such as a lupus condition) may be greater than the second number of independent training samples associated with an absence of the condition (e.g., a disease or disorder, such as a lupus condition).

The trained algorithm may comprise a classifier configured to identify the presence (e.g., positive test result) or absence (e.g., negative test result) of one or more conditions (e.g., a disease or disorder, such as a lupus condition) at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the presence (e.g., positive test result) or absence (e.g., negative test result) of the one or more conditions by the trained algorithm may be calculated as the percentage of independent test samples (e.g., subjects known to have the condition or subjects with negative clinical test results for the condition) that are correctly identified or classified as having or not having the condition.

The trained algorithm may comprise a classifier configured to identify one or more conditions (e.g., a disease or disorder, such as a lupus condition) with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the condition using the trained algorithm may be calculated as the percentage of samples identified or classified as having the condition that correspond to subjects that truly have the condition.

The trained algorithm may comprise a classifier configured to identify one or more conditions (e.g., a disease or disorder, such as a lupus condition) with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the condition using the trained algorithm may be calculated as the percentage of samples identified or classified as not having the condition that correspond to subjects that truly do not have the condition.

The trained algorithm may comprise a classifier configured to identify one or more conditions (e.g., a disease or disorder, such as a lupus condition) with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the condition using the trained algorithm may be calculated as the percentage of independent test samples associated with presence of the condition (e.g., subjects known to have the condition) that are correctly identified or classified as having the condition.

The trained algorithm may comprise a classifier configured to identify one or more conditions (e.g., a disease or disorder, such as a lupus condition) with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the condition using the trained algorithm may be calculated as the percentage of independent test samples associated with absence of the condition (e.g., subjects with negative clinical test results for the condition) that are correctly identified or classified as not having the condition.

The trained algorithm may comprise a classifier configured to identify the presence (e.g., positive test result) or absence (e.g., negative test result) of one or more conditions (e.g., a disease or disorder, such as a lupus condition) with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying samples as having or not having the condition.

Classifiers of the trained algorithm may be adjusted or tuned to improve or optimize one or more performance metrics, such as accuracy, PPV, NPV, clinical sensitivity, clinical specificity, AUC, or a combination thereof (e.g., a performance index incorporating a plurality of such performance metrics, such as by calculating a weight sum therefrom), of identifying the presence (e.g., positive test result) or absence (e.g., negative test result) of the condition. The classifiers may be adjusted or tuned by adjusting parameters of the classifiers (e.g., a set of cutoff values used to classify a sample as described elsewhere herein, or weights of a neural network) to improve or optimize the performance metrics. The one or more classifiers may be adjusted or tuned so as to reduce an overall classification error (e.g., an “out-of-bag” or oob error rate for a Random Forest classifier). The one or more classifiers may be adjusted or tuned continuously during the training process (e.g., as sample datasets are added to the training set) or after the training process has completed.

The trained algorithm may comprise a plurality of classifiers (e.g., an ensemble) such that the plurality of classifications or outcome values of the plurality of classifiers may be combined to produce a single classification or outcome value for the sample. For example, a sum or a weighted sum of the plurality of classifications or outcome values of the plurality of classifiers may be calculated to produce a single classification or outcome value for the sample. As another example, a majority vote of the plurality of classifications or outcome values of the plurality of classifiers may be identified to produce a single classification or outcome value for the sample. In this manner, a single classification or outcome value may be produced for the sample having greater confidence or statistical significance than the individual classifications or outcome values produced by each of the plurality of classifiers.

After the trained algorithm is initially trained, a subset of the inputs may be identified as most influential or most important to be included for making high-quality classifications (e.g., having highest permutation feature importance). For example, a subset of the panel of lupus condition-associated or LDG-associated genomic loci may be identified as most influential or most important to be included for making high-quality classifications or identifications of conditions (or sub-types of conditions). The panel of lupus condition-associated or LDG-associated genomic loci, or a subset thereof, may be ranked based on classification metrics indicative of each influence or importance of each individual lupus condition-associated or LDG-associated genomic locus toward making high-quality classifications or identifications of conditions (or sub-types of conditions). Such metrics may be used to reduce, in some cases significantly, the number of input variables (e.g., predictor variables) that may be used to train the one or more classifiers of the trained algorithm to a desired performance level (e.g., based on a desired minimum accuracy, PPV, NPV, clinical sensitivity, clinical specificity, AUC, or a combination thereof).

For example, if training a classifier of the trained algorithm with a plurality comprising several dozen or hundreds of input variables to the classifier results in an accuracy of classification of more than 99%, then training the classifier of the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

As another example, if training a classifier of the trained algorithm with a plurality comprising several dozen or hundreds of input variables to the classifier results in a sensitivity or specificity of classification of more than 99%, then training the classifier of the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable sensitivity or specificity of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

The subset of the plurality of input variables (e.g., the panel of lupus condition-associated or LDG-associated genomic loci) to the classifier of the trained algorithm may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics (e.g., permutation feature importance).

Upon identifying the subject as having one or more conditions (e.g., a disease or disorder, such as a lupus condition), the subject may be optionally provided with a therapeutic intervention (e.g., prescribing an appropriate course of treatment to treat the one or more conditions of the subject). The therapeutic intervention may comprise a prescription of an effective dose of a drug, a further testing or evaluation of the condition, a further monitoring of the condition, or a combination thereof. If the subject is currently being treated for the condition with a course of treatment, the therapeutic intervention may comprise a subsequent different course of treatment (e.g., to increase treatment efficacy due to non-efficacy of the current course of treatment).

The therapeutic intervention may include prescribed medications or drugs, which may include one or more of: antimalarials, corticosteroids, immunosuppressants, and nonsteroidal anti-inflammatory drugs (NSAIDs). The therapeutic intervention may be effective to alleviate or decrease one or more symptoms, which may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof.

The therapeutic intervention may comprise recommending the subject for a secondary clinical test to confirm a diagnosis of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

The feature sets (e.g., comprising quantitative measures of a panel of lupus condition-associated or LDG-associated genomic loci) may be analyzed and assessed (e.g., using a trained algorithm comprising one or more classifiers) over a duration of time to monitor a patient (e.g., subject who has a condition or who is being treated for a condition). In such cases, the feature sets of the patient may change during the course of treatment. For example, the quantitative measures of the feature sets of a patient with decreasing risk of the condition due to an effective treatment may shift toward the profile or distribution of a healthy subject (e.g., a subject without the condition). Conversely, for example, the quantitative measures of the feature sets of a patient with increasing risk of the condition due to an ineffective treatment may shift toward the profile or distribution of a subject with higher risk of the condition or a more advanced stage or severity of the condition.

The condition of the subject may be monitored by monitoring a course of treatment for treating the condition of the subject. The monitoring may comprise assessing the condition of the subject at two or more time points. The assessing may be based at least on the feature sets (e.g., quantitative measures of a panel of lupus condition-associated or LDG-associated genomic loci) determined at each of the two or more time points. The therapeutic intervention may include prescribed medications or drugs, which may include one or more of: antimalarials, corticosteroids, immunosuppressants, and nonsteroidal anti-inflammatory drugs (NSAIDs). The therapeutic intervention may be effective to alleviate or decrease one or more symptoms, which may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof. The assessing may be based at least on the presence, absence, or severity of one or more symptoms, such as alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof.

In some embodiments, a difference in the feature sets (e.g., quantitative measures of a panel of lupus condition-associated or LDG-associated genomic loci) determined between the two or more time points may be indicative of one or more clinical indications, such as (i) a diagnosis of the condition of the subject, (ii) a prognosis of the condition of the subject, (iii) an increased risk of the condition of the subject, (iv) a decreased risk of the condition of the subject, (v) an efficacy of the course of treatment for treating the condition of the subject, and (vi) a non-efficacy of the course of treatment for treating the condition of the subject.

In some embodiments, a difference in the feature sets (e.g., quantitative measures of a panel of lupus condition-associated or LDG-associated genomic loci) determined between the two or more time points may be indicative of a diagnosis of the condition of the subject. For example, if the condition was not detected in the subject at an earlier time point but was detected in the subject at a later time point, then the difference is indicative of a diagnosis of the condition of the subject. A clinical action or decision may be made based on this indication of diagnosis of the condition of the subject, such as, for example, prescribing a new therapeutic intervention for the subject. The clinical action or decision may comprise recommending the subject for a secondary clinical test to confirm the diagnosis of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of lupus condition-associated or LDG-associated genomic loci) determined between the two or more time points may be indicative of a prognosis of the condition of the subject.

In some embodiments, a difference in the feature sets (e.g., quantitative measures of a panel of lupus condition-associated or LDG-associated genomic loci) determined between the two or more time points may be indicative of the subject having an increased risk of the condition. For example, if the condition was detected in the subject both at an earlier time point and at a later time point, and if the difference is a negative difference (e.g., the quantitative measures of a panel of lupus condition-associated or LDG-associated genomic loci increased from the earlier time point to the later time point), then the difference may be indicative of the subject having an increased risk of the condition. A clinical action or decision may be made based on this indication of the increased risk of the condition, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of lupus condition-associated or LDG-associated genomic loci) determined between the two or more time points may be indicative of the subject having a decreased risk of the condition. For example, if the condition was detected in the subject both at an earlier time point and at a later time point, and if the difference is a positive difference (e.g., the quantitative measures of a panel of lupus condition-associated or LDG-associated genomic loci decreased from the earlier time point to the later time point), then the difference may be indicative of the subject having a decreased risk of the condition. A clinical action or decision may be made based on this indication of the decreased risk of the condition (e.g., continuing or ending a current therapeutic intervention) for the subject. The clinical action or decision may comprise recommending the subject for a secondary clinical test to confirm the decreased risk of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of lupus condition-associated or LDG-associated genomic loci) determined between the two or more time points may be indicative of an efficacy of the course of treatment for treating the condition of the subject. For example, if the condition was detected in the subject at an earlier time point but was not detected in the subject at a later time point, then the difference may be indicative of an efficacy of the course of treatment for treating the condition of the subject. A clinical action or decision may be made based on this indication of the efficacy of the course of treatment for treating the condition of the subject, e.g., continuing or ending a current therapeutic intervention for the subject. The clinical action or decision may comprise recommending the subject for a secondary clinical test to confirm the efficacy of the course of treatment for treating the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of lupus condition-associated or LDG-associated genomic loci) determined between the two or more time points may be indicative of a non-efficacy of the course of treatment for treating the condition of the subject. For example, if the condition was detected in the subject both at an earlier time point and at a later time point, and if the difference is a negative or zero difference (e.g., the quantitative measures of a panel of lupus condition-associated or LDG-associated genomic loci increased or remained at a constant level from the earlier time point to the later time point), and if an efficacious treatment was indicated at an earlier time point, then the difference may be indicative of a non-efficacy of the course of treatment for treating the condition of the subject. A clinical action or decision may be made based on this indication of the non-efficacy of the course of treatment for treating the condition of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In various embodiments, machine learning methods are applied to distinguish samples in a population of samples. In one embodiment, machine learning methods are applied to distinguish samples between healthy and lupus (e.g., SLE or DLE) samples.

Kits

The present disclosure provides kits for identifying or monitoring a disease or disorder (e.g., a lupus condition) of a subject. A kit may comprise probes for identifying a quantitative measure (e.g., indicative of a presence, absence, or relative amount) of sequences at each of a panel of lupus condition-associated or LDG-associated genomic loci in a sample of the subject. A quantitative measure (e.g., indicative of a presence, absence, or relative amount) of sequences at each of a panel of lupus condition-associated or LDG-associated genomic loci in the sample may be indicative of the disease or disorder (e.g., a lupus condition) of the subject. The probes may be selective for the sequences at the panel of lupus condition-associated or LDG-associated genomic loci in the sample. A kit may comprise instructions for using the probes to process the sample to generate datasets indicative of a quantitative measure (e.g., indicative of a presence, absence, or relative amount) of sequences at each of the panel of lupus condition-associated or LDG-associated genomic loci in a sample of the subject.

The probes in the kit may be selective for the sequences at the panel of lupus condition-associated or LDG-associated genomic loci in the sample. The probes in the kit may be configured to selectively enrich nucleic acid (e.g., RNA or DNA) molecules corresponding to the panel of lupus condition-associated or LDG-associated genomic loci. The probes in the kit may be nucleic acid primers. The probes in the kit may have sequence complementarity with nucleic acid sequences from one or more of the panel of lupus condition-associated or LDG-associated genomic loci. The panel of lupus condition-associated or LDG-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more distinct lupus condition-associated or LDG-associated genomic loci.

The instructions in the kit may comprise instructions to assay the sample using the probes that are selective for the sequences at the panel of lupus condition-associated or LDG-associated genomic loci in the cell-free biological sample. These probes may be nucleic acid molecules (e.g., RNA or DNA) having sequence complementarity with nucleic acid sequences (e.g., RNA or DNA) from one or more of the plurality of panel of lupus condition-associated or LDG-associated genomic loci. These nucleic acid molecules may be primers or enrichment sequences. The instructions to assay the cell-free biological sample may comprise introductions to perform array hybridization, polymerase chain reaction (PCR), or nucleic acid sequencing (e.g., DNA sequencing or RNA sequencing) to process the sample to generate datasets indicative of a quantitative measure (e.g., indicative of a presence, absence, or relative amount) of sequences at each of the panel of lupus condition-associated or LDG-associated genomic loci in the sample. A quantitative measure (e.g., indicative of a presence, absence, or relative amount) of sequences at each of a panel of lupus condition-associated or LDG-associated genomic loci in the sample may be indicative of a disease or disorder (e.g., a lupus condition).

The instructions in the kit may comprise instructions to measure and interpret assay readouts, which may be quantified at one or more of the panel of lupus condition-associated or LDG-associated genomic loci to generate the datasets indicative of a quantitative measure (e.g., indicative of a presence, absence, or relative amount) of sequences at each of the panel of lupus condition-associated or LDG-associated genomic loci in the sample. For example, quantification of array hybridization or polymerase chain reaction (PCR) corresponding to the panel of lupus condition-associated or LDG-associated genomic loci may generate the datasets indicative of a quantitative measure (e.g., indicative of a presence, absence, or relative amount) of sequences at each of the panel of lupus condition-associated or LDG-associated genomic loci in the sample. Assay readouts may comprise quantitative PCR (qPCR) values, digital PCR (dPCR) values, digital droplet PCR (ddPCR) values, fluorescence values, etc., or normalized values thereof.

### Primary Immunodeficiency (PID) Profiling of Lupus Conditions

Systemic lupus erythematosus (SLE) may be a polygenic autoimmune disease defined by hyper-reactivity of the immune system. In healthy individuals, the immune system may protect the host from invading microorganisms. However, subjects (e.g., patients) with primary immunodeficiency (PID) may not be able to generate an effective immune response and hence may suffer from repeated infections. Bioinformatic approaches may use gene expression data and clinical measurements to generate a transcriptomic signature that characterizes PID in SLE, toward understanding the relationship between this signature and SLE disease manifestations.

To examine checkpoints in the immune system driving autoimmunity in SLE, sets of genes abnormally expressed in SLE cells may be compared to sets of causal genes underlying PID. A hypothesis that genes “knocked out” in PID are overexpressed in lupus, and therefore possibly contributing to the immune over-reactivity, may be tested. After compiling a comprehensive database of genes discovered through this process, some of the the PID-associated genes may be observed to be differentially expressed (DE) in SLE. Further, some of the the PID-associated genes may be found to be uniquely DE in immune subsets (e.g., myeloid, T cells, NK cells, B cells, plasma cells, and neutrophils). A variety of bioinformatics tools may be employed to elucidate the nature of the PID-associated genes that were over-expressed in SLE. For example, STRING, a protein-protein interaction analytic tool, may be applied to the dataset, and distinct groups (e.g., clusters) of PID-associated genes may be identified. Further, Gene Set Variation Analysis (GSVA) may be applied to the dataset, and distinct gene clusters may be identified to be enriched in a set of SLE patients. Clusters of PID-associated genes may be observed to be consistently enriched (e.g., interferon stimulated genes, MHC class-1 antigen presentation, secreted-immune, secreted extracellular matrix, pattern recognition receptors, proteasome activity, and pro-apoptosis). These results may establish that the non-redundant checkpoint genes underlying PID are over-expressed in SLE patients. These genes and the pathways they identify may be used as unique targets for novel therapies in SLE.

The results obtained may provide a deeper understanding of the relationship between primary immunodeficiency (PID) genes and a specific autoimmune disorder, systemic lupus erythematosus (SLE). SLE is a complex genetically-based autoimmune disease defined by the production of high affinity autoantibodies that cause damage to tissues and may be lethal. SLE may disproportionately affect certain groups of subjects (e.g., patients), such as females of African ancestry, and may include exacerbations and great variability. PID may be considered as essentially the functional inactivation of the immune system, in which the causal genes are biological upstream regulators. If a particular gene is knocked out in a subject, then a severe immune phenotype may persist, and the subject's susceptibility to recurrent infections may increase significantly. On the other hand, autoimmunity generally arises in a subject from the over-activation of the immune system of the subject. Therefore, PID and autoimmunity may be considered as opposite sides of the same coin.

In some cases, PID and autoimmunity may share the loss of regulatory checkpoints in the immune system, and these checkpoints may be governed by the same genes. Instead of examining the entire human genome, identified PID-associated genes were analyzed, and their role in SLE was elucidated. For example, PID-associated genes may be identified and the role of these genes in SLE may be analyzed, e.g., by cross-referencing differential expression datasets and utilizing various analytical tools to understand the common genes between SLE and PID. Due to the complexity of SLE, many types of drugs (e.g., antimalarial, corticosteroids, immunosuppressants, biologics, and nonsteroidal anti-inflammatory drugs) may be utilized to treat symptoms. Belimumab (Benlysta®), the only drug approved in 60 years to treat SLE, is a biologic that inhibits the binding of B cells to B lymphocyte stimulators. Identified PID-associated genes that are also marker genes for SLE may be explored as potential drug therapy targets for SLE patients.

In an aspect, the present disclosure provides a method for identifying a lupus condition of a subject, comprising: (a) assaying a biological sample of the subject to generate a dataset comprising gene expression data; (b) processing the dataset at each of a plurality of genes to determine quantitative measures of each of the plurality of genes, wherein the plurality of genes comprises primary immunodeficiency (PID)-associated genes, thereby producing a PID signature of the biological sample of the subject; (c) processing the PID signature with one or more reference PID signatures, wherein the processing comprises, for at least one of the plurality of genes, comparing the quantitative measure of the gene of the PID signature with corresponding quantitative measures of the gene of the one or more reference PID signatures; (d) based at least in part on the comparison in (c), identifying the lupus condition of the subject.

In some embodiments, the lupus condition is selected from the group consisting of: systemic lupus erythematosus (SLE), discoid lupus erythematosus (DLE), and lupus nephritis (LN). In some embodiments, the biological sample is selected from the group consisting of: a whole blood (WB) sample, a PBMC sample, a tissue sample, and a cell sample. In some embodiments, the tissue sample is selected from the group consisting of: skin tissue, synovium tissue, kidney tissue, and bone marrow tissue. In some embodiments, the kidney tissue is selected from the group consisting of: glomerulus (Glom) and tubulointerstitium (TI). In some embodiments, the cell sample is selected from the group consisting of: myelocytes (MY), promyelocytes (PM), polymorphonuclear neutrophils (PMN), peripheral blood mononuclear cells (PBMC), and hematopoietic stem cells.

In some embodiments, the method further comprises enriching or purifying a whole blood sample of the subject to obtain the cell sample. In some embodiments, assaying the biological sample comprises (i) using a microarray to generate the dataset comprising the gene expression data, (ii) sequencing the biological sample to generate the dataset comprising the gene expression data, or (iii) performing quantitative polymerase chain reaction (qPCR) of the biological sample to generate the dataset comprising the gene expression data.

In some embodiments, the plurality of genes comprises PID-associated genes selected from the genes listed in Table 47. In some embodiments, the plurality of genes comprises at least 5 PID-associated genes selected from the genes listed in Table 47. In some embodiments, the plurality of genes comprises at least 10 PID-associated genes selected from the genes listed in Table 47. In some embodiments, the plurality of genes comprises at least 25 PID-associated genes selected from the genes listed in Table 47. In some embodiments, the plurality of genes comprises at least 50 PID-associated genes selected from the genes listed in Table 47. In some embodiments, the plurality of genes comprises at least 100 PID-associated genes selected from the genes listed in Table 47.

In some embodiments, the quantitative measures of each of the plurality of genes comprise enrichment scores of each of the plurality of genes. In some embodiments, the enrichment scores of each of the plurality of genes comprise gene set variation analysis (GSVA) enrichment scores of each of the plurality of genes. In some embodiments, (c) further comprises, for the at least one of the plurality of genes, determining a difference between the quantitative measure of the gene of the PID signature with the corresponding quantitative measures of the gene of the one or more reference PID signatures. In some embodiments, (d) further comprises identifying the lupus condition of the subject when the difference satisfies a pre-determined criterion.

In some embodiments, (c) further comprises, for the at least one of the plurality of genes, determining a Z-score of the quantitative measure of the gene of the PID signature relative to the corresponding quantitative measures of the gene of the one or more reference PID signatures. In some embodiments, (d) further comprises identifying the lupus condition of the subject when the Z-score satisfies a pre-determined criterion. In some embodiments, (d) further comprises identifying the lupus condition of the subject when the Z-score is at least about 3, and identifying an absence of the lupus condition of the subject when the Z-score is less than about 3. In some embodiments, (d) further comprises identifying the lupus condition of the subject when the Z-score is at least about 2.5, and identifying an absence of the lupus condition of the subject when the Z-score is less than about 2.5. In some embodiments, (d) further comprises identifying the lupus condition of the subject when the Z-score is at least about 2, and identifying an absence of the lupus condition of the subject when the Z-score is less than about 2. In some embodiments, (d) further comprises identifying the lupus condition of the subject when the Z-score is at least about 1.5, and identifying an absence of the lupus condition of the subject when the Z-score is less than about 1.5. In some embodiments, (d) further comprises identifying the lupus condition of the subject when the Z-score is at least about 1, and identifying an absence of the lupus condition of the subject when the Z-score is less than about 1. In some embodiments, (d) further comprises identifying the lupus condition of the subject when the Z-score is at least about 0.5, and identifying an absence of the lupus condition of the subject when the Z-score is less than about 0.5.

In some embodiments, the method further comprises identifying the lupus condition of the subject at a sensitivity of at least about 60%. In some embodiments, the method further comprises identifying the lupus condition of the subject at a sensitivity of at least about 65%. In some embodiments, the method further comprises identifying the lupus condition of the subject at a sensitivity of at least about 70%. In some embodiments, the method further comprises identifying the lupus condition of the subject at a sensitivity of at least about 75%. In some embodiments, the method further comprises identifying the lupus condition of the subject at a sensitivity of at least about 80%. In some embodiments, the method further comprises identifying the lupus condition of the subject at a sensitivity of at least about 85%. In some embodiments, the method further comprises identifying the lupus condition of the subject at a sensitivity of at least about 90%. In some embodiments, the method further comprises identifying the lupus condition of the subject at a sensitivity of at least about 95%. In some embodiments, the method further comprises identifying the lupus condition of the subject at a sensitivity of at least about 99%.

In some embodiments, the method further comprises identifying the lupus condition of the subject at a specificity of at least about 60%. In some embodiments, the method further comprises identifying the lupus condition of the subject at a specificity of at least about 65%. In some embodiments, the method further comprises identifying the lupus condition of the subject at a specificity of at least about 70%. In some embodiments, the method further comprises identifying the lupus condition of the subject at a specificity of at least about 75%. In some embodiments, the method further comprises identifying the lupus condition of the subject at a specificity of at least about 80%. In some embodiments, the method further comprises identifying the lupus condition of the subject at a specificity of at least about 85%. In some embodiments, the method further comprises identifying the lupus condition of the subject at a specificity of at least about 90%. In some embodiments, the method further comprises identifying the lupus condition of the subject at a specificity of at least about 95%. In some embodiments, the method further comprises identifying the lupus condition of the subject at a specificity of at least about 99%.

In some embodiments, the method further comprises identifying the lupus condition of the subject at a positive predictive value (PPV) of at least about 60%. In some embodiments, the method further comprises identifying the lupus condition of the subject at a positive predictive value (PPV) of at least about 65%. In some embodiments, the method further comprises identifying the lupus condition of the subject at a positive predictive value (PPV) of at least about 70%. In some embodiments, the method further comprises identifying the lupus condition of the subject at a positive predictive value (PPV) of at least about 75%. In some embodiments, the method further comprises identifying the lupus condition of the subject at a positive predictive value (PPV) of at least about 80%. In some embodiments, the method further comprises identifying the lupus condition of the subject at a positive predictive value (PPV) of at least about 85%. In some embodiments, the method further comprises identifying the lupus condition of the subject at a positive predictive value (PPV) of at least about 90%. In some embodiments, the method further comprises identifying the lupus condition of the subject at a positive predictive value (PPV) of at least about 95%. In some embodiments, the method further comprises identifying the lupus condition of the subject at a positive predictive value (PPV) of at least about 99%.

In some embodiments, the method further comprises identifying the lupus condition of the subject at a negative predictive value (NPV) of at least about 60%. In some embodiments, the method further comprises identifying the lupus condition of the subject at a negative predictive value (NPV) of at least about 65%. In some embodiments, the method further comprises identifying the lupus condition of the subject at a negative predictive value (NPV) of at least about 70%. In some embodiments, the method further comprises identifying the lupus condition of the subject at a negative predictive value (NPV) of at least about 75%. In some embodiments, the method further comprises identifying the lupus condition of the subject at a negative predictive value (NPV) of at least about 80%. In some embodiments, the method further comprises identifying the lupus condition of the subject at a negative predictive value (NPV) of at least about 85%. In some embodiments, the method further comprises identifying the lupus condition of the subject at a negative predictive value (NPV) of at least about 90%. In some embodiments, the method further comprises identifying the lupus condition of the subject at a negative predictive value (NPV) of at least about 95%. In some embodiments, the method further comprises identifying the lupus condition of the subject at a negative predictive value (NPV) of at least about 99%.

In some embodiments, the method further comprises identifying the lupus condition of the subject with an Area Under Curve (AUC) of at least about 0.60. In some embodiments, the method further comprises identifying the lupus condition of the subject with an Area Under Curve (AUC) of at least about 0.65. In some embodiments, the method further comprises identifying the lupus condition of the subject with an Area Under Curve (AUC) of at least about 0.70. In some embodiments, the method further comprises identifying the lupus condition of the subject with an Area Under Curve (AUC) of at least about 0.75. In some embodiments, the method further comprises identifying the lupus condition of the subject with an Area Under Curve (AUC) of at least about 0.80. In some embodiments, the method further comprises identifying the lupus condition of the subject with an Area Under Curve (AUC) of at least about 0.85. In some embodiments, the method further comprises identifying the lupus condition of the subject with an Area Under Curve (AUC) of at least about 0.90. In some embodiments, the method further comprises identifying the lupus condition of the subject with an Area Under Curve (AUC) of at least about 0.95. In some embodiments, the method further comprises identifying the lupus condition of the subject with an Area Under Curve (AUC) of at least about 0.99.

In some embodiments, (d) further comprises identifying the lupus condition of the subject based at least in part on a SLEDAI score of the subject. In some embodiments, the subject is asymptomatic for one or more lupus conditions selected from the group consisting of: systemic lupus erythematosus (SLE), discoid lupus erythematosus (DLE), and lupus nephritis (LN).

In some embodiments, the method further comprises applying a trained algorithm to the PID signature to identify the lupus condition of the subject. In some embodiments, the trained algorithm is trained using a first set of independent training samples associated with a presence of the lupus condition and a second set of independent training samples associated with an absence of the lupus condition. In some embodiments, the method further comprises using the trained algorithm to process a set of clinical health data of the subject to identify the lupus condition. In some embodiments, the trained algorithm comprises a supervised machine learning algorithm. In some embodiments, the supervised machine learning algorithm comprises a deep learning algorithm, a support vector machine (SVM), a neural network, or a Random Forest.

In some embodiments, (a) comprises (i) subjecting the biological sample to conditions that are sufficient to isolate, enrich, or extract a plurality of nucleic acid molecules; and (ii) analyzing the plurality of nucleic acid molecules to generate the dataset comprising the gene expression data.

In some embodiments, the method further comprises using probes configured to selectively enrich the plurality of nucleic acid molecules corresponding to a panel of one or more genomic loci. In some embodiments, the probes are nucleic acid primers. In some embodiments, the probes have sequence complementarity with nucleic acid sequences of the panel of the one or more genomic loci. In some embodiments, the panel of the one or more genomic loci comprises genomic loci corresponding to the plurality of genes. In some embodiments, the panel of said one or more genomic loci comprises at least 5 distinct genomic loci. In some embodiments, the panel of said one or more genomic loci comprises at least 10 distinct genomic loci. In some embodiments, the panel of said one or more genomic loci comprises at least 25 distinct genomic loci. In some embodiments, the panel of said one or more genomic loci comprises at least 50 distinct genomic loci. In some embodiments, the panel of said one or more genomic loci comprises at least 100 distinct genomic loci. In some embodiments, the panel of said one or more genomic loci comprises at least 150 distinct genomic loci.

In some embodiments, the method further comprises (e) assaying a second biological sample of the subject to generate a second dataset comprising gene expression data; (f) processing the second dataset at each of the plurality of genes to determine second quantitative measures of each of the plurality of genes, thereby producing a second PID signature of the second biological sample of the subject; (g) processing the second PID signature with one or more reference PID signatures, wherein the processing comprises, for at least one of the plurality of genes, comparing the quantitative measure of the gene of the second PID signature with corresponding quantitative measures of the gene of the one or more reference PID signatures; and (h) based at least in part on the comparison in (g), identifying the lupus condition of the subject.

In some embodiments, the biological sample and the second biological sample comprise two different sample types selected from the group consisting of: a whole blood (WB) sample, a PBMC sample, a skin tissue sample, a synovium tissue sample, a kidney tissue sample comprising glomerulus (Glom), a kidney tissue sample comprising tubulointerstitium (TI), a bone marrow tissue, a myelocyte (MY) cell sample, a promyelocyte (PM) cell sample, a polymorphonuclear neutrophils (PMN) sample, and a hematopoietic stem cell sample.

In some embodiments, the method further comprises determining a likelihood of the identification of the lupus condition of the subject. In some embodiments, the method further comprises providing a therapeutic intervention for the lupus condition of the subject.

In some embodiments, the method further comprises monitoring the lupus condition of the subject, wherein the monitoring comprises assessing the lupus condition of the subject at a plurality of time points, wherein the assessing is based at least on the lupus condition identified in (d) at each of the plurality of time points.

In some embodiments, a difference in the assessment of the lupus condition of the subject among the plurality of time points is indicative of one or more clinical indications selected from the group consisting of: (i) a diagnosis of the lupus condition of the subject, (ii) a prognosis of the lupus condition of the subject, and (iii) an efficacy or non-efficacy of a course of treatment for treating the lupus condition of the subject.

In some embodiments, the one or more reference PID signatures are generated by: assaying a biological sample of one or more patients having one or more disease symptoms or being treated with one or more drugs to generate a reference dataset comprising gene expression data; and processing the reference dataset at each of the plurality of genes to determine quantitative measures of each of the plurality of genes.

In some embodiments, the one or more disease symptoms are selected from the group consisting of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance.

In some embodiments, the one or more drugs are selected from the group consisting of: antimalarials, corticosteroids, immunosuppressants, and nonsteroidal anti-inflammatory drugs (NSAIDs).

In another aspect, the present disclosure provides a computer system for identifying a lupus condition of a subject, comprising: a database that is configured to store a dataset comprising gene expression data, wherein the gene expression data is obtained by assaying a biological sample of the subject; and one or more computer processors operatively coupled to the database, wherein the one or more computer processors are individually or collectively programmed to: (i) process the dataset at each of a plurality of genes to determine quantitative measures of each of the plurality of genes, wherein the plurality of genes comprises primary immunodeficiency (PID)-associated genes, thereby producing a PID signature of the biological sample of the subject; (ii) process the PID signature with one or more reference PID signatures, wherein the processing comprises, for at least one of the plurality of genes, comparing the quantitative measure of the gene of the PID signature with corresponding quantitative measures of the gene of the one or more reference PID signatures; and (iii) based at least in part on the comparison in (ii), identify the lupus condition of the subject.

In some embodiments, computer system further comprises an electronic display operatively coupled to the one or more computer processors, wherein the electronic display comprises a graphical user interface that is configured to display the report.

In another aspect, the present disclosure provides a non-transitory computer readable medium comprising machine-executable code that, upon execution by one or more computer processors, implements a method for identifying a lupus condition of a subject, the method comprising: (a) assaying a biological sample of the subject to generate a dataset comprising gene expression data; (b) processing the dataset at each of a plurality of genes to determine quantitative measures of each of the plurality of genes, wherein the plurality of genes comprises primary immunodeficiency (PID)-associated genes, thereby producing a PID signature of the biological sample of the subject; (c) processing the PID signature with one or more reference PID signatures, wherein the processing comprises, for at least one of the plurality of genes, comparing the quantitative measure of the gene of the PID signature with corresponding quantitative measures of the gene of the one or more reference PID signatures; (d) based at least in part on the comparison in (c), identifying the lupus condition of the subject.

To obtain a blood sample, various techniques may be used, e.g., a syringe or other vacuum suction device. A blood sample can be optionally pre-treated or processed prior to use. A sample, such as a blood sample, may be analyzed under any of the methods and systems herein within 4 weeks, 2 weeks, 1 week, 6 days, 5 days, 4 days, 3 days, 2 days, 1 day, 12 hr, 6 hr, 3 hr, 2 hr, or 1 hr from the time the sample is obtained, or longer if frozen. When obtaining a sample from a subject (e.g., blood sample), the amount can vary depending upon subject size and the condition being screened. In some embodiments, at least 10 mL, 5 mL, 1 mL, 0.5 mL, 250, 200, 150, 100, 50, 40, 30, 20, 10, 9, 8, 7, 6, 5, 4, 3, 2, or 1 μL of a sample is obtained. In some embodiments, 1-50, 2-40, 3-30, or 4-20 μL of sample is obtained. In some embodiments, more than 5, 10, 15, 20, 25, 30, 35, 40, 45, 50, 55, 60, 65, 70, 75, 80, 85, 90, 95 or 100 μL of a sample is obtained.

The sample may be taken before and/or after treatment of a subject with a disease or disorder. Samples may be obtained from a subject during a treatment or a treatment regime. Multiple samples may be obtained from a subject to monitor the effects of the treatment over time. The sample may be taken from a subject known or suspected of having a disease or disorder for which a definitive positive or negative diagnosis is not available via clinical tests. The sample may be taken from a subject suspected of having a disease or disorder. The sample may be taken from a subject experiencing unexplained symptoms, such as fatigue, nausea, weight loss, aches and pains, weakness, or bleeding. The sample may be taken from a subject having explained symptoms. The sample may be taken from a subject at risk of developing a disease or disorder due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

In some embodiments, a sample can be taken at a first time point and assayed, and then another sample can be taken at a subsequent time point and assayed. Such methods can be used, for example, for longitudinal monitoring purposes to track the development or progression of a disease. In some embodiments, the progression of a disease can be tracked before treatment, after treatment, or during the course of treatment, to determine the treatment's effectiveness. For example, a method as described herein can be performed on a subject prior to, and after, treatment with a lupus condition therapy to measure the disease's progression or regression in response to the lupus condition therapy.

After obtaining a sample from the subject, the sample may be processed to generate datasets indicative of a disease or disorder of the subject. For example, a presence, absence, or quantitative assessment of nucleic acid molecules of the sample at a panel of lupus condition-associated or PID-associated genomic loci or may be indicative of a lupus condition of the subject. Processing the sample obtained from the subject may comprise (i) subjecting the sample to conditions that are sufficient to isolate, enrich, or extract a plurality of nucleic acid molecules, and (ii) assaying the plurality of nucleic acid molecules to generate the dataset (e.g., microarray data, nucleic acid sequences, or quantitative polymerase chain reaction (qPCR) data). Methods of assaying may include any assay known in the art or described in the literature, for example, a microarray assay, a sequencing assay (e.g., DNA sequencing, RNA sequencing, or RNA-Seq), or a quantitative polymerase chain reaction (qPCR) assay.

In some embodiments, a plurality of nucleic acid molecules is extracted from the sample and subjected to sequencing to generate a plurality of sequencing reads. The nucleic acid molecules may comprise ribonucleic acid (RNA) or deoxyribonucleic acid (DNA). The extraction method may extract all RNA or DNA molecules from a sample. Alternatively, the extraction method may selectively extract a portion of RNA or DNA molecules from a sample. Extracted RNA molecules from a sample may be converted to cDNA molecules by reverse transcription (RT).

The sample may be processed without any nucleic acid extraction. For example, the disease or disorder may be identified or monitored in the subject by using probes configured to selectively enrich nucleic acid (e.g., RNA or DNA) molecules corresponding to a panel of lupus condition-associated or PID-associated genomic loci. The probes may be nucleic acid primers. The probes may have sequence complementarity with nucleic acid sequences from one or more of the panel of lupus condition-associated or PID-associated genomic loci. The panel of lupus condition-associated or PID-associated genomic loci may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more lupus condition-associated or PID-associated genomic loci.

The probes may be nucleic acid molecules (e.g., RNA or DNA) having sequence complementarity with nucleic acid sequences (e.g., RNA or DNA) of one or more genomic loci (e.g., lupus condition-associated or PID-associated genomic loci). These nucleic acid molecules may be primers or enrichment sequences. The assaying of the sample using probes that are selective for the one or more genomic loci (e.g., lupus condition-associated or PID-associated genomic loci) may comprise use of array hybridization, polymerase chain reaction (PCR), or nucleic acid sequencing (e.g., RNA sequencing or DNA sequencing, such as RNA-Seq).

The assay readouts may be quantified at one or more genomic loci (e.g., lupus condition-associated or PID-associated genomic loci) to generate the data indicative of the disease or disorder. For example, quantification of array hybridization or polymerase chain reaction (PCR) corresponding to a plurality of genomic loci (e.g., lupus condition-associated or PID-associated genomic loci) may generate data indicative of the disease or disorder. Assay readouts may comprise quantitative PCR (qPCR) values, digital PCR (dPCR) values, digital droplet PCR (ddPCR) values, fluorescence values, etc., or normalized values thereof

Methods

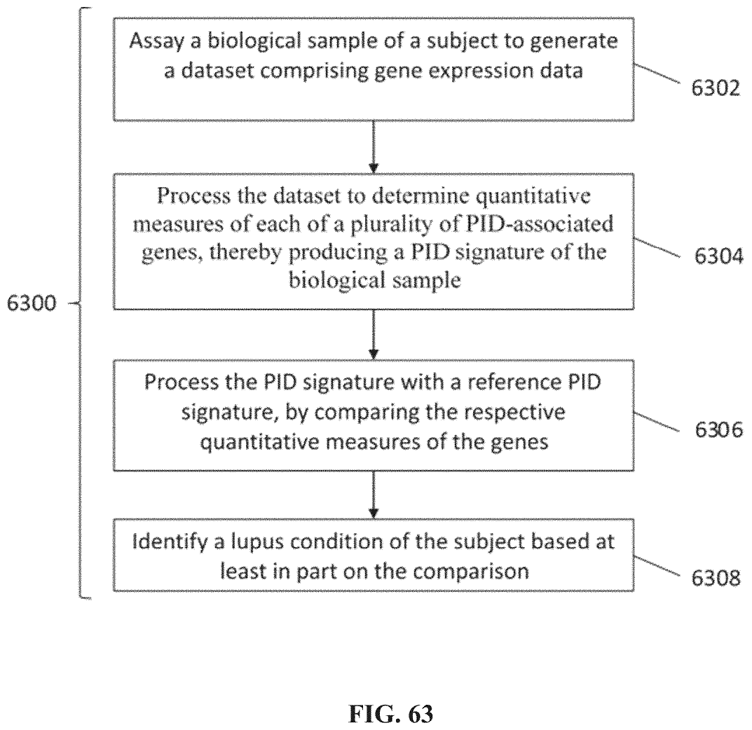

FIG. 63 shows a non-limiting example of a method 6300 for identifying a lupus condition of a subject using PID profiling, in accordance with disclosed embodiments. The method may comprise assaying a biological sample of a subject to generate a dataset comprising gene expression data (as in 6302). Next, the method may comprise processing the dataset to determine quantitative measures of each of a plurality of PID-associated genes, thereby producing a PID signature of the biological sample (as in 6304). Next, the method may comprise processing the PID signature with a reference PID signature (as in 6306). For example, the processing may be performed by comparing the respective quantitative measures of the genes of the PID signature and the reference PID signature. Next, the method may comprise identifying the lupus condition of the subject based at least in part on the comparison (as in 6308).

A database of PID-associated genes may be constructed as follows. Once identified via thorough searches of primary scientific literature on PIDs, a plurality of causal genes may be compiled into a database. The database may include one or more of the following information for each gene: Gene Symbol, Official Symbol, Full Name, Functional Category (BIG-C™) Entrez ID, Ensembl ID, Gene Type, Synonyms, Chromosome Number, Cytogenetic Location, Inheritance, genetic Defect/Pathogenesis, Phenotype, Relevance to SLE, Allelic Mutations (OMIM and Primary literature), Protein Effect (GeneCards), OMIM Gene ID, OMIM Phenotype ID, and Mendelian Genetics ID.

BIG-C™ analysis may be performed on the data as follows. Biologically Informed Gene Clustering (BIG-CTM) is a functional aggregating tool (AMPEL BioSolutions, Charlottesville, Va.) for analyzing and understanding the biological groupings of large lists of genes. Genes are sorted into 45 categories based on their most likely biological function and/or cellular localization based on information from multiple online tools and databases.

I-SCOPE analysis may be performed on the data as follows. PID-associated genes may be cross-referenced with immune genes restrictively expressed in hematopoietic genes restrictively expressed in hematopoietic cells using the I-SCOPE tool (AMPEL BioSolutions, Charlottesville, Va.).

Cytoscape, STRING, and MCODE analyses may be performed on the data as follows. A visualization of protein-protein interactions and relationships between genes within datasets may be performed using the Cytoscape (V3.6.0) software and the MCODE StringApp (V1.3.2) plugin application. The Clustermaker2 App (V1.2.1) plugin may be used to create clusters of the most related genes within a dataset, using a network scoring degree cutoff of 2 and setting a node score cut-off of 0.2, k-Core of 2, and a max depth of 100.

Gene expression data may be compiled from SLE patients as follows. Data may be derived from publicly available datasets and collaborators. Raw data files may be obtained from the GEO repository for SLE whole blood data. The following datasets may be used: GSE22098, GSE39088, GSE88884, GSE45291, and GSE61635.

The data may be analyzed for differential gene expression (e.g., between SLE patients vs. controls) as follows. GCRMA normalized expression values may be variance corrected using local empirical Bayesian shrinkage, followed by calculation of DE using the ebayes function in the BioConductor LIMMA package. Resulting p-values may be adjusted for multiple hypothesis testing and filtered to retain DE probes with an FDR<0.2.

Gene Set Variation Analysis (GSVA) may be performed on the data as follows. The GSVA (V1.25.0) software package for R/Bioconductor may be used as a non-parametric, unsupervised method for estimating the variation of pre-defined gene sets in patient and control samples of microarray expression data sets. GSVA may be run using GSE88884 and the MCODE Clusters.

Hedge's G values, a measure of effect size, may be calculated from the GSVA enrichment scores, by contrasting K-S scores of all controls against all lupus patient samples. GSVA enrichment scores may be additionally utilized for Welch's t-tests to identify significant (e.g., p<0.05) gene categories contributing to substantial segregation of cohort samples. Results may be visualized by using a matrix of Hedge's G values was entered as input to the corplot package of R (dual scale heatmap). Significant categories may be identified (e.g., having a statistically significant degree of DE).

Classifiers

In some embodiments, the present disclosure provides a system, method, or kit having data analysis realized in software application, computing hardware, or both. In various embodiments, the analysis application or system includes at least a data receiving module, a data pre-processing module, a data analysis module, a data interpretation module, or a data visualization module. In one embodiment, the data receiving module can comprise computer systems that connect laboratory hardware or instrumentation with computer systems that process laboratory data. In one embodiment, the data pre-processing module can comprise hardware systems or computer software that performs operations on the data in preparation for analysis. Examples of operations that can be applied to the data in the pre-processing module include affine transformations, denoising operations, data cleaning, reformatting, or subsampling. A data analysis module, which can be specialized for analyzing genomic data from one or more genomic materials, can, for example, take assembled genomic sequences and perform probabilistic and statistical analysis to identify abnormal patterns related to a disease, pathology, state, risk, condition, or phenotype. A data interpretation module can use analysis methods, for example, drawn from statistics, mathematics, or biology, to support understanding of the relation between the identified abnormal patterns and health conditions, functional states, prognoses, or risks. A data visualization module can use methods of mathematical modeling, computer graphics, or rendering to create visual representations of data that can facilitate the understanding or interpretation of results.

Feature sets may be generated from datasets obtained using one or more assays of a biological sample, and a trained algorithm may be used to process one or more of the feature sets to identify or assess the condition (e.g., a disease or disorder, such as a lupus condition). For example, the trained algorithm may be used to apply a machine learning classifier to a plurality of lupus condition-associated or PID-associated genomic loci that are associated with two or more classes of individuals inputted into a machine learning model, in order to classify a subject into one of the two or more classes of individuals. For example, the trained algorithm may be used to apply a machine learning classifier to a plurality of lupus condition-associated or PID-associated genomic loci that are associated with individuals with known conditions (e.g., a disease or disorder, such as a lupus condition) and individuals not having the condition (e.g., healthy individuals, or individuals who do not have a lupus condition), in order to classify a subject as having the condition (e.g., positive test outcome) or not having the condition (e.g., negative test outcome).

The trained algorithm may be configured to identify the presence (e.g., positive test result) or absence (e.g., negative test result) of one or more conditions (e.g., a disease or disorder, such as a lupus condition) with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99%. This accuracy may be achieved for a set of at least about 25, at least about 50, at least about 100, at least about 150, at least about 200, at least about 250, at least about 300, at least about 350, at least about 400, at least about 450, at least about 500, at least about 1,000, or more than about 1,000 independent samples.

The trained algorithm may comprise a machine learning algorithm, such as a supervised machine learning algorithm. The supervised machine learning algorithm may comprise, for example, a Random Forest, a support vector machine (SVM), a neural network, or a deep learning algorithm. The trained algorithm may comprise a classification and regression tree (CART) algorithm. The trained algorithm may comprise an unsupervised machine learning algorithm.

The trained algorithm may comprise a classifier configured to accept as input a plurality of input variables or features (e.g., lupus condition-associated or PID-associated genomic loci) and to produce or output one or more output values based on the plurality of input variables or features (e.g., lupus condition-associated or PID-associated genomic loci). The plurality of input variables or features may comprise one or more datasets indicative of the presence (e.g., positive test result) or absence (e.g., negative test result) of one or more conditions (e.g., a disease or disorder, such as a lupus condition). For example, an input variable or feature may comprise a number of sequences corresponding to or aligning to each of the plurality of lupus condition-associated or PID-associated genomic loci.

The plurality of input variables or features may also include clinical information of a subject, such as health data. For example, the health data of a subject may comprise one or more of: a diagnosis of one or more conditions (e.g., a disease or disorder, such as a lupus condition), a prognosis of one or more conditions (e.g., a disease or disorder, such as a lupus condition), a risk of having one or more conditions (e.g., a disease or disorder, such as a lupus condition), a treatment history of one or more conditions (e.g., a disease or disorder, such as a lupus condition), a history of previous treatment for one or more conditions (e.g., a disease or disorder, such as a lupus condition), a history of prescribed medications, a history of prescribed medical devices, age, height, weight, sex, smoking status, and one or more symptoms of the subject.

For example, the disease or disorder may comprise one or more of: systemic lupus erythematosus (SLE), discoid lupus erythematosus (DLE), and lupus nephritis (LN). As another example, the symptoms may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof. As another example, the prescribed medications or drugs may include one or more of: antimalarials, corticosteroids, immunosuppressants, and nonsteroidal anti-inflammatory drugs (NSAIDs).

The trained algorithm may comprise a classifier, such that each of the one or more output values comprises one of a fixed number of possible values (e.g., a linear classifier, a logistic regression classifier, etc.) indicating a classification of the sample by the classifier. The trained algorithm may comprise a binary classifier, such that each of the one or more output values comprises one of two values (e.g., {0, 1}, {positive, negative}, or {high-risk, low-risk}) indicating a classification of the sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the sample by the classifier.

The classifier may be configured to classify samples by assigning output values, which may comprise descriptive labels, numerical values, or a combination thereof. Some of the output values may comprise descriptive labels. Such descriptive labels may provide an identification or indication of the presence (e.g., positive test result) or absence (e.g., negative test result) of one or more conditions (e.g., a disease or disorder, such as a lupus condition) of the subject, and may comprise, for example, positive, negative, high-risk, intermediate-risk, low-risk, or indeterminate. Such descriptive labels may provide an identification of a treatment for the one or more conditions of the subject, and may comprise, for example, a therapeutic intervention, a duration of the therapeutic intervention, and/or a dosage of the therapeutic intervention suitable to treat the one or more conditions of the subject. Such descriptive labels may provide an identification of secondary clinical tests that may be appropriate to perform on the subject, and may comprise, for example, an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof. For example, such descriptive labels may provide a prognosis of the one or more conditions of the subject. As another example, such descriptive labels may provide a relative assessment of the one or more conditions of the subject. Some descriptive labels may be mapped to numerical values, for example, by mapping “positive” to 1 and “negative” to 0.

The classifier may be configured to classify samples by assigning output values that comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the one or more conditions (e.g., a disease or disorder, such as a lupus condition) of the subject. Some numerical values may be mapped to descriptive labels, for example, by mapping 1 to “positive” and 0 to “negative.”

The classifier may be configured to classify samples by assigning output values based on one or more cutoff values. For example, a binary classification of samples may assign an output value of “positive” or 1 if the sample indicates that the subject has at least a 50% probability of having one or more conditions (e.g., a disease or disorder, such as a lupus condition), thereby assigning the subject to a class of individuals receiving a positive test result. As another example, a binary classification of samples may assign an output value of “negative” or 0 if the sample indicates that the subject has less than a 50% probability of having one or more conditions (e.g., a disease or disorder), thereby assigning the subject to a class of individuals receiving a negative test result. In this case, a single cutoff value of 50% is used to classify samples into one of the two possible binary output values or classes of individuals (e.g., those receiving a positive test result and those receiving a negative test result). Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, the classifier may be configured to classify samples by assigning an output value of “positive” or 1 if the sample indicates that the subject has a probability of having one or more conditions (e.g., a disease or disorder, such as a lupus condition) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of having one or more conditions (e.g., a disease or disorder, such as a lupus condition) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classifier may be configured to classify samples by assigning an output value of “negative” or 0 if the sample indicates that the subject has a probability of having one or more conditions (e.g., a disease or disorder, such as a lupus condition) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having one or more conditions (e.g., a disease or disorder, such as a lupus condition) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classifier may be configured to classify samples by assigning an output value of “indeterminate” or 2 if the sample is not classified as “positive”, “negative”, 1, or 0. In this case, a set of two cutoff values is used to classify samples into one of the three possible output values or classes of individuals (e.g., corresponding to outcome groups of individuals having “low risk,” “intermediate risk,” and “high risk” of having one or more conditions, such as a disease or disorder). Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values or classes of individuals, where n is any positive integer.

The trained algorithm may be trained with a plurality of independent training samples. Each of the independent training samples may comprise a sample from a subject, associated datasets obtained by assaying the sample (as described elsewhere herein), and one or more known output values or classes of individuals corresponding to the sample (e.g., a clinical diagnosis, prognosis, absence, or treatment efficacy of a condition of the subject). Independent training samples may comprise samples and associated datasets and outputs obtained or derived from a plurality of different subjects. Independent training samples may comprise samples and associated datasets and outputs obtained at a plurality of different time points from the same subject (e.g., on a regular basis such as weekly, biweekly, or monthly), as part of a longitudinal monitoring of a subject before, during, and after a course of treatment for one or more conditions of the subject. Independent training samples may be associated with presence of the condition (e.g., training samples comprising samples and associated datasets and outputs obtained or derived from a plurality of subjects known to have the condition). Independent training samples may be associated with absence of the condition (e.g., training samples comprising samples and associated datasets and outputs obtained or derived from a plurality of subjects who are known to not have a previous diagnosis of the condition or who have received a negative test result for the condition).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise samples associated with presence of the condition and/or samples associated with absence of the condition. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the condition (e.g., a disease or disorder, such as a lupus condition). The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with absence of the condition (e.g., a disease or disorder, such as a lupus condition). In some embodiments, the sample is independent of samples used to train the trained algorithm.

The trained algorithm may be trained with a first number of independent training samples associated with a presence of the condition (e.g., a disease or disorder, such as a lupus condition) and a second number of independent training samples associated with an absence of the condition (e.g., a disease or disorder, such as a lupus condition). The first number of independent training samples associated with presence of the condition (e.g., a disease or disorder, such as a lupus condition) may be no more than the second number of independent training samples associated with absence of the condition (e.g., a disease or disorder, such as a lupus condition). The first number of independent training samples associated with a presence of the condition (e.g., a disease or disorder) may be equal to the second number of independent training samples associated with an absence of the condition (e.g., a disease or disorder, such as a lupus condition). The first number of independent training samples associated with a presence of the condition (e.g., a disease or disorder, such as a lupus condition) may be greater than the second number of independent training samples associated with an absence of the condition (e.g., a disease or disorder, such as a lupus condition).

The trained algorithm may comprise a classifier configured to identify the presence (e.g., positive test result) or absence (e.g., negative test result) of one or more conditions (e.g., a disease or disorder, such as a lupus condition) at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the presence (e.g., positive test result) or absence (e.g., negative test result) of the one or more conditions by the trained algorithm may be calculated as the percentage of independent test samples (e.g., subjects known to have the condition or subjects with negative clinical test results for the condition) that are correctly identified or classified as having or not having the condition.

The trained algorithm may comprise a classifier configured to identify one or more conditions (e.g., a disease or disorder, such as a lupus condition) with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the condition using the trained algorithm may be calculated as the percentage of samples identified or classified as having the condition that correspond to subjects that truly have the condition.

The trained algorithm may comprise a classifier configured to identify one or more conditions (e.g., a disease or disorder, such as a lupus condition) with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the condition using the trained algorithm may be calculated as the percentage of samples identified or classified as not having the condition that correspond to subjects that truly do not have the condition.

The trained algorithm may comprise a classifier configured to identify one or more conditions (e.g., a disease or disorder, such as a lupus condition) with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the condition using the trained algorithm may be calculated as the percentage of independent test samples associated with presence of the condition (e.g., subjects known to have the condition) that are correctly identified or classified as having the condition.

The trained algorithm may comprise a classifier configured to identify one or more conditions (e.g., a disease or disorder, such as a lupus condition) with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the condition using the trained algorithm may be calculated as the percentage of independent test samples associated with absence of the condition (e.g., subjects with negative clinical test results for the condition) that are correctly identified or classified as not having the condition.

The trained algorithm may comprise a classifier configured to identify the presence (e.g., positive test result) or absence (e.g., negative test result) of one or more conditions (e.g., a disease or disorder, such as a lupus condition) with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying samples as having or not having the condition.

Classifiers of the trained algorithm may be adjusted or tuned to improve or optimize one or more performance metrics, such as accuracy, PPV, NPV, clinical sensitivity, clinical specificity, AUC, or a combination thereof (e.g., a performance index incorporating a plurality of such performance metrics, such as by calculating a weight sum therefrom), of identifying the presence (e.g., positive test result) or absence (e.g., negative test result) of the condition. The classifiers may be adjusted or tuned by adjusting parameters of the classifiers (e.g., a set of cutoff values used to classify a sample as described elsewhere herein, or weights of a neural network) to improve or optimize the performance metrics. The one or more classifiers may be adjusted or tuned so as to reduce an overall classification error (e.g., an “out-of-bag” or oob error rate for a Random Forest classifier). The one or more classifiers may be adjusted or tuned continuously during the training process (e.g., as sample datasets are added to the training set) or after the training process has completed.

The trained algorithm may comprise a plurality of classifiers (e.g., an ensemble) such that the plurality of classifications or outcome values of the plurality of classifiers may be combined to produce a single classification or outcome value for the sample. For example, a sum or a weighted sum of the plurality of classifications or outcome values of the plurality of classifiers may be calculated to produce a single classification or outcome value for the sample. As another example, a majority vote of the plurality of classifications or outcome values of the plurality of classifiers may be identified to produce a single classification or outcome value for the sample. In this manner, a single classification or outcome value may be produced for the sample having greater confidence or statistical significance than the individual classifications or outcome values produced by each of the plurality of classifiers.

After the trained algorithm is initially trained, a subset of the inputs may be identified as most influential or most important to be included for making high-quality classifications (e.g., having highest permutation feature importance). For example, a subset of the panel of lupus condition-associated or PID-associated genomic loci may be identified as most influential or most important to be included for making high-quality classifications or identifications of conditions (or sub-types of conditions). The panel of lupus condition-associated or PID-associated genomic loci, or a subset thereof, may be ranked based on classification metrics indicative of each influence or importance of each individual lupus condition-associated or PID-associated genomic locus toward making high-quality classifications or identifications of conditions (or sub-types of conditions). Such metrics may be used to reduce, in some cases significantly, the number of input variables (e.g., predictor variables) that may be used to train the one or more classifiers of the trained algorithm to a desired performance level (e.g., based on a desired minimum accuracy, PPV, NPV, clinical sensitivity, clinical specificity, AUC, or a combination thereof).

For example, if training a classifier of the trained algorithm with a plurality comprising several dozen or hundreds of input variables to the classifier results in an accuracy of classification of more than 99%, then training the classifier of the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

As another example, if training a classifier of the trained algorithm with a plurality comprising several dozen or hundreds of input variables to the classifier results in a sensitivity or specificity of classification of more than 99%, then training the classifier of the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable sensitivity or specificity of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

The subset of the plurality of input variables (e.g., the panel of lupus condition-associated or PID-associated genomic loci) to the classifier of the trained algorithm may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics (e.g., permutation feature importance).

Upon identifying the subject as having one or more conditions (e.g., a disease or disorder, such as a lupus condition), the subject may be optionally provided with a therapeutic intervention (e.g., prescribing an appropriate course of treatment to treat the one or more conditions of the subject). The therapeutic intervention may comprise a prescription of an effective dose of a drug, a further testing or evaluation of the condition, a further monitoring of the condition, or a combination thereof. If the subject is currently being treated for the condition with a course of treatment, the therapeutic intervention may comprise a subsequent different course of treatment (e.g., to increase treatment efficacy due to non-efficacy of the current course of treatment).

The therapeutic intervention may include prescribed medications or drugs, which may include one or more of: antimalarials, corticosteroids, immunosuppressants, and nonsteroidal anti-inflammatory drugs (NSAIDs). The therapeutic intervention may be effective to alleviate or decrease one or more symptoms, which may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof.

The therapeutic intervention may comprise recommending the subject for a secondary clinical test to confirm a diagnosis of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

The feature sets (e.g., comprising quantitative measures of a panel of lupus condition-associated or PID-associated genomic loci) may be analyzed and assessed (e.g., using a trained algorithm comprising one or more classifiers) over a duration of time to monitor a patient (e.g., subject who has a condition or who is being treated for a condition). In such cases, the feature sets of the patient may change during the course of treatment. For example, the quantitative measures of the feature sets of a patient with decreasing risk of the condition due to an effective treatment may shift toward the profile or distribution of a healthy subject (e.g., a subject without the condition). Conversely, for example, the quantitative measures of the feature sets of a patient with increasing risk of the condition due to an ineffective treatment may shift toward the profile or distribution of a subject with higher risk of the condition or a more advanced stage or severity of the condition.

The condition of the subject may be monitored by monitoring a course of treatment for treating the condition of the subject. The monitoring may comprise assessing the condition of the subject at two or more time points. The assessing may be based at least on the feature sets (e.g., quantitative measures of a panel of lupus condition-associated or PID-associated genomic loci) determined at each of the two or more time points. The therapeutic intervention may include prescribed medications or drugs, which may include one or more of: antimalarials, corticosteroids, immunosuppressants, and nonsteroidal anti-inflammatory drugs (NSAIDs). The therapeutic intervention may be effective to alleviate or decrease one or more symptoms, which may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof. The assessing may be based at least on the presence, absence, or severity of one or more symptoms, such as alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof.

In some embodiments, a difference in the feature sets (e.g., quantitative measures of a panel of lupus condition-associated or PID-associated genomic loci) determined between the two or more time points may be indicative of one or more clinical indications, such as (i) a diagnosis of the condition of the subject, (ii) a prognosis of the condition of the subject, (iii) an increased risk of the condition of the subject, (iv) a decreased risk of the condition of the subject, (v) an efficacy of the course of treatment for treating the condition of the subject, and (vi) a non-efficacy of the course of treatment for treating the condition of the subject.

In some embodiments, a difference in the feature sets (e.g., quantitative measures of a panel of lupus condition-associated or PID-associated genomic loci) determined between the two or more time points may be indicative of a diagnosis of the condition of the subject. For example, if the condition was not detected in the subject at an earlier time point but was detected in the subject at a later time point, then the difference is indicative of a diagnosis of the condition of the subject. A clinical action or decision may be made based on this indication of diagnosis of the condition of the subject, such as, for example, prescribing a new therapeutic intervention for the subject. The clinical action or decision may comprise recommending the subject for a secondary clinical test to confirm the diagnosis of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of lupus condition-associated or PID-associated genomic loci) determined between the two or more time points may be indicative of a prognosis of the condition of the subject.

In some embodiments, a difference in the feature sets (e.g., quantitative measures of a panel of lupus condition-associated or PID-associated genomic loci) determined between the two or more time points may be indicative of the subject having an increased risk of the condition. For example, if the condition was detected in the subject both at an earlier time point and at a later time point, and if the difference is a positive difference (e.g., the quantitative measures of a panel of lupus condition-associated or PID-associated genomic loci increased from the earlier time point to the later time point), then the difference may be indicative of the subject having an increased risk of the condition. A clinical action or decision may be made based on this indication of the increased risk of the condition, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of lupus condition-associated or PID-associated genomic loci) determined between the two or more time points may be indicative of the subject having a decreased risk of the condition. For example, if the condition was detected in the subject both at an earlier time point and at a later time point, and if the difference is a negative difference (e.g., the quantitative measures of a panel of lupus condition-associated or PID-associated genomic loci decreased from the earlier time point to the later time point), then the difference may be indicative of the subject having a decreased risk of the condition. A clinical action or decision may be made based on this indication of the decreased risk of the condition (e.g., continuing or ending a current therapeutic intervention) for the subject. The clinical action or decision may comprise recommending the subject for a secondary clinical test to confirm the decreased risk of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of lupus condition-associated or PID-associated genomic loci) determined between the two or more time points may be indicative of an efficacy of the course of treatment for treating the condition of the subject. For example, if the condition was detected in the subject at an earlier time point but was not detected in the subject at a later time point, then the difference may be indicative of an efficacy of the course of treatment for treating the condition of the subject. A clinical action or decision may be made based on this indication of the efficacy of the course of treatment for treating the condition of the subject, e.g., continuing or ending a current therapeutic intervention for the subject. The clinical action or decision may comprise recommending the subject for a secondary clinical test to confirm the efficacy of the course of treatment for treating the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of lupus condition-associated or PID-associated genomic loci) determined between the two or more time points may be indicative of a non-efficacy of the course of treatment for treating the condition of the subject. For example, if the condition was detected in the subject both at an earlier time point and at a later time point, and if the difference is a positive or zero difference (e.g., the quantitative measures of a panel of lupus condition-associated or PID-associated genomic loci increased or remained at a constant level from the earlier time point to the later time point), and if an efficacious treatment was indicated at an earlier time point, then the difference may be indicative of a non-efficacy of the course of treatment for treating the condition of the subject. A clinical action or decision may be made based on this indication of the non-efficacy of the course of treatment for treating the condition of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In various embodiments, machine learning methods are applied to distinguish samples in a population of samples. In one embodiment, machine learning methods are applied to distinguish samples between healthy and lupus (e.g., SLE or DLE) samples.

Kits

The present disclosure provides kits for identifying or monitoring a disease or disorder (e.g., a lupus condition) of a subject. A kit may comprise probes for identifying a quantitative measure (e.g., indicative of a presence, absence, or relative amount) of sequences at each of a panel of lupus condition-associated or PID-associated genomic loci in a sample of the subject. A quantitative measure (e.g., indicative of a presence, absence, or relative amount) of sequences at each of a panel of lupus condition-associated or PID-associated genomic loci in the sample may be indicative of the disease or disorder (e.g., a lupus condition) of the subject. The probes may be selective for the sequences at the panel of lupus condition-associated or PID-associated genomic loci in the sample. A kit may comprise instructions for using the probes to process the sample to generate datasets indicative of a quantitative measure (e.g., indicative of a presence, absence, or relative amount) of sequences at each of the panel of lupus condition-associated or PID-associated genomic loci in a sample of the subject.

The probes in the kit may be selective for the sequences at the panel of lupus condition-associated or PID-associated genomic loci in the sample. The probes in the kit may be configured to selectively enrich nucleic acid (e.g., RNA or DNA) molecules corresponding to the panel of lupus condition-associated or PID-associated genomic loci. The probes in the kit may be nucleic acid primers. The probes in the kit may have sequence complementarity with nucleic acid sequences from one or more of the panel of lupus condition-associated or PID-associated genomic loci. The panel of lupus condition-associated or PID-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more distinct lupus condition-associated or PID-associated genomic loci.

The instructions in the kit may comprise instructions to assay the sample using the probes that are selective for the sequences at the panel of lupus condition-associated or PID-associated genomic loci in the cell-free biological sample. These probes may be nucleic acid molecules (e.g., RNA or DNA) having sequence complementarity with nucleic acid sequences (e.g., RNA or DNA) from one or more of the panel of lupus condition-associated or PID-associated genomic loci. These nucleic acid molecules may be primers or enrichment sequences. The instructions to assay the cell-free biological sample may comprise introductions to perform array hybridization, polymerase chain reaction (PCR), or nucleic acid sequencing (e.g., DNA sequencing or RNA sequencing) to process the sample to generate datasets indicative of a quantitative measure (e.g., indicative of a presence, absence, or relative amount) of sequences at each of the panel of lupus condition-associated or PID-associated genomic loci in the sample. A quantitative measure (e.g., indicative of a presence, absence, or relative amount) of sequences at each of a panel of lupus condition-associated or PID-associated genomic loci in the sample may be indicative of a disease or disorder (e.g., a lupus condition).

The instructions in the kit may comprise instructions to measure and interpret assay readouts, which may be quantified at one or more of the panel of lupus condition-associated or PID-associated genomic loci to generate the datasets indicative of a quantitative measure (e.g., indicative of a presence, absence, or relative amount) of sequences at each of the panel of lupus condition-associated or PID-associated genomic loci in the sample. For example, quantification of array hybridization or polymerase chain reaction (PCR) corresponding to the panel of lupus condition-associated or PID-associated genomic loci may generate the datasets indicative of a quantitative measure (e.g., indicative of a presence, absence, or relative amount) of sequences at each of the panel of lupus condition-associated or PID-associated genomic loci in the sample. Assay readouts may comprise quantitative PCR (qPCR) values, digital PCR (dPCR) values, digital droplet PCR (ddPCR) values, fluorescence values, etc., or normalized values thereof.

### Biological Data Analysis

The present disclosure provides systems and methods to perform data analysis using drug or target scoring algorithms and/or big data analysis tools. In various aspects, such drug or target scoring algorithms and/or big data analysis tools may be used to perform analysis of data sets including, for example, mRNA gene expression or transcriptome data, DNA genomic data, proteomic data, metabolomic data, other types of “-omic” data, or a combination thereof.

In an aspect, the present disclosure provides a computer-implemented method for assessing a condition of a subject, comprising: (a) receiving a dataset of a biological sample of the subject; (b) selecting one or more data analysis tools, wherein the one or more data analysis tools comprise an analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool; (c) processing the dataset using the one or more data analysis tools to generate a data signature of the biological sample of the subject; and (d) based at least in part on the data signature generated in (c), assessing the condition of the subject.

In some embodiments, the dataset comprises mRNA gene expression or transcriptome data, DNA genomic data, proteomic data, metabolomic data, or a combination thereof. In some embodiments, the biological sample is selected from the group consisting of: a whole blood (WB) sample, a PBMC sample, a tissue sample, and a cell sample. In some embodiments, assessing the condition of the subject comprises identifying a disease or disorder of the subject.

In some embodiments, the method further comprises identifying a disease or disorder of the subject at a sensitivity or specificity of at least about 70%. In some embodiments, the method further comprises determining a likelihood of the identification of the disease or disorder of the subject. In some embodiments, the method further comprises providing a therapeutic intervention for the disease or disorder of the subject. In some embodiments, the method further comprises monitoring the disease or disorder of the subject, wherein the monitoring comprises assessing the disease or disorder of the subject at a plurality of time points, wherein the assessing is based at least on the disease or disorder identified at each of the plurality of time points.

In some embodiments, selecting the one or more data analysis tools comprises receiving a user selection of the one or more data analysis tools. In some embodiments, selecting the one or more data analysis tools is automatically performed by the computer without receiving a user selection of the one or more data analysis tools.

In another aspect, the present disclosure provides a computer system for assessing a condition of a subject, comprising: a database that is configured to store a dataset of a biological sample of the subject; and one or more computer processors operatively coupled to the database, wherein the one or more computer processors are individually or collectively programmed to: (i) select one or more data analysis tools comprising: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, a Target Scoring analysis tool, or a combination thereof; (ii) process the dataset using the one or more data analysis tools to generate a data signature of the biological sample of the subject; and (iii) based at least in part on the data signature generated in (ii), assess the condition of the subject.

In another aspect, the present disclosure provides a non-transitory computer readable medium comprising machine-executable code that, upon execution by one or more computer processors, implements a method for assessing a condition of a subject, the method comprising: (a) receiving a dataset of a biological sample of the subject; (b) selecting one or more data analysis tools, wherein the one or more data analysis tools comprise an analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool; (c) processing the dataset using the one or more data analysis tools to generate a data signature of the biological sample of the subject; and (d) based at least in part on the data signature generated in (c), assessing the condition of the subject. In any embodiment described herein, the one or more data analysis tools can be a plurality of data analysis tools each independently selected from a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool.

To obtain a blood sample, various techniques may be used, e.g., a syringe or other vacuum suction device. A blood sample can be optionally pre-treated or processed prior to use. A sample, such as a blood sample, may be analyzed under any of the methods and systems herein within 4 weeks, 2 weeks, 1 week, 6 days, 5 days, 4 days, 3 days, 2 days, 1 day, 12 hr, 6 hr, 3 hr, 2 hr, or 1 hr from the time the sample is obtained, or longer if frozen. When obtaining a sample from a subject (e.g., blood sample), the amount can vary depending upon subject size and the condition being screened. In some embodiments, at least 10 mL, 5 mL, 1 mL, 0.5 mL, 250, 200, 150, 100, 50, 40, 30, 20, 10, 9, 8, 7, 6, 5, 4, 3, 2, or 1 μL of a sample is obtained. In some embodiments, 1-50, 2-40, 3-30, or 4-20 μL of sample is obtained. In some embodiments, more than 5, 10, 15, 20, 25, 30, 35, 40, 45, 50, 55, 60, 65, 70, 75, 80, 85, 90, 95 or 100 μL of a sample is obtained.

The sample may be taken before and/or after treatment of a subject with a disease or disorder. Samples may be obtained from a subject during a treatment or a treatment regime. Multiple samples may be obtained from a subject to monitor the effects of the treatment over time. The sample may be taken from a subject known or suspected of having a disease or disorder for which a definitive positive or negative diagnosis is not available via clinical tests. The sample may be taken from a subject suspected of having a disease or disorder. The sample may be taken from a subject experiencing unexplained symptoms, such as fatigue, nausea, weight loss, aches and pains, weakness, or bleeding. The sample may be taken from a subject having explained symptoms. The sample may be taken from a subject at risk of developing a disease or disorder due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

In some embodiments, a sample can be taken at a first time point and assayed, and then another sample can be taken at a subsequent time point and assayed. Such methods can be used, for example, for longitudinal monitoring purposes to track the development or progression of a disease. In some embodiments, the progression of a disease can be tracked before treatment, after treatment, or during the course of treatment, to determine the treatment's effectiveness. For example, a method as described herein can be performed on a subject prior to, and after, treatment with a lupus condition therapy to measure the disease's progression or regression in response to the lupus condition therapy.

After obtaining a sample from the subject, the sample may be processed to generate datasets indicative of a disease or disorder of the subject. For example, a presence, absence, or quantitative assessment of nucleic acid molecules of the sample at a panel of condition-associated genomic loci or may be indicative of a lupus condition of the subject. Processing the sample obtained from the subject may comprise (i) subjecting the sample to conditions that are sufficient to isolate, enrich, or extract a plurality of nucleic acid molecules, and (ii) assaying the plurality of nucleic acid molecules to generate the dataset (e.g., microarray data, nucleic acid sequences, or quantitative polymerase chain reaction (qPCR) data). Methods of assaying may include any assay known in the art or described in the literature, for example, a microarray assay, a sequencing assay (e.g., DNA sequencing, RNA sequencing, or RNA-Seq), or a quantitative polymerase chain reaction (qPCR) assay.

In some embodiments, a plurality of nucleic acid molecules is extracted from the sample and subjected to sequencing to generate a plurality of sequencing reads. The nucleic acid molecules may comprise ribonucleic acid (RNA) or deoxyribonucleic acid (DNA). The extraction method may extract all RNA or DNA molecules from a sample. Alternatively, the extraction method may selectively extract a portion of RNA or DNA molecules from a sample. Extracted RNA molecules from a sample may be converted to cDNA molecules by reverse transcription (RT).

The sample may be processed without any nucleic acid extraction. For example, the disease or disorder may be identified or monitored in the subject by using probes configured to selectively enrich nucleic acid (e.g., RNA or DNA) molecules corresponding to a panel of condition-associated genomic loci. The probes may be nucleic acid primers. The probes may have sequence complementarity with nucleic acid sequences from one or more of the panel of condition-associated genomic loci. The panel of condition-associated genomic loci may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more condition-associated genomic loci.

The probes may be nucleic acid molecules (e.g., RNA or DNA) having sequence complementarity with nucleic acid sequences (e.g., RNA or DNA) of one or more genomic loci (e.g., condition-associated genomic loci). These nucleic acid molecules may be primers or enrichment sequences. The assaying of the sample using probes that are selective for the one or more genomic loci (e.g., condition-associated genomic loci) may comprise use of array hybridization, polymerase chain reaction (PCR), or nucleic acid sequencing (e.g., RNA sequencing or DNA sequencing, such as RNA-Seq).

The assay readouts may be quantified at one or more genomic loci (e.g., condition-associated genomic loci) to generate the data indicative of the disease or disorder. For example, quantification of array hybridization or polymerase chain reaction (PCR) corresponding to a plurality of genomic loci (e.g., condition-associated genomic loci) may generate data indicative of the disease or disorder. Assay readouts may comprise quantitative PCR (qPCR) values, digital PCR (dPCR) values, digital droplet PCR (ddPCR) values, fluorescence values, etc., or normalized values thereof.

Big Data Analysis Tools and Drug/Target Scoring Algorithms

The present disclosure provides systems and methods to perform data analysis using drug or target scoring algorithms and/or big data analysis tools. In various aspects, such drug or target scoring algorithms and/or big data analysis tools may be used to perform analysis of data sets including, for example, mRNA gene expression or transcriptome data, DNA genomic data, proteomic data, metabolomic data, other types of “-omic” data, or a combination thereof. Systems and methods of the present disclosure may use one or more of the following: a BIG-CTM big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool.

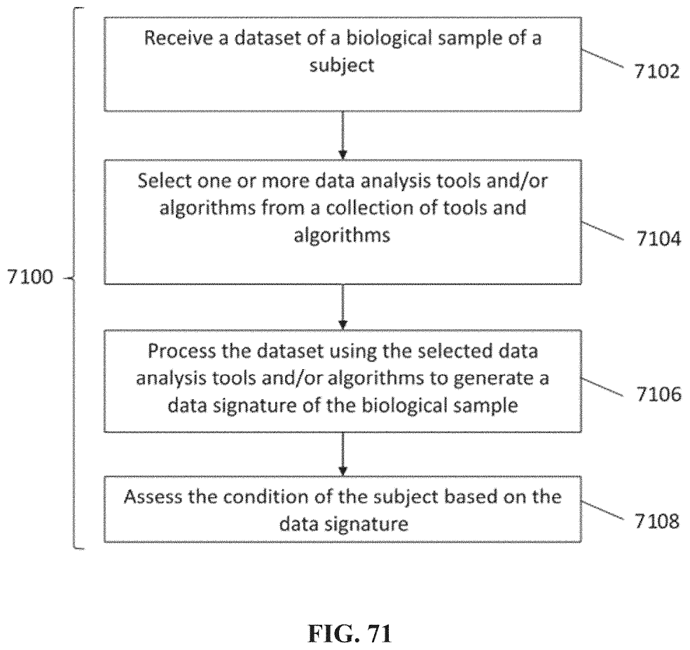

FIG. 71 shows a non-limiting example of a workflow of a method 7100 to assess a condition of a subject using one or more data analysis tools and/or algorithms. The method may comprise receiving a dataset of a biological sample of a subject (as in 7102). Next, the method may comprise selecting one or more data analysis tools and/or algorithms (as in 7104). For example, the data analysis tools and/or algorithms may comprise a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, a Target Scoring analysis tool, or a combination thereof. Next, the method may comprise processing the dataset using selected data analysis tools and/or algorithms to generate a data signature of the biological sample of the subject (as in 7106). Next, the method may comprise assessing the condition of the subject based on the data signature (as in 7108).

The BIG-C (Biologically Informed Gene Clustering) tool may be configured to sort large groups of genes into a set of functional groups (e.g., 53 functional groups). The functional groups are created utilizing publicly available information from online tools and databases including UniProtKB/Swiss-Prot, GO Terms, KEGG pathways, NCBI PubMed, and the Interactome. The functional groups may include one or more of: Active RNA, Anti-apoptosis, anti-proliferation, autophagy, chromatin remodeling, cytoplasm and biochemistry, cytoskeleton, DNA repair, endocytosis, endoplasmic reticulum, endosome and vesicles, fatty acid biosynthesis, cell surface, transcription, glycolysis and gluconeogenesis, golgi, immune cell surface, immune secreted, immune signaling, integrin pathway, interferon stimulated genes, intracellular signaling, lysosome, melanosome, MHC class I, MHC class II, microRNA processing, microRNA, mitochondrial transcription, mitochondria, mitochondria oxidative phosphorylation, mitochondrial TCA cycle, mRNA processing, mRNA splicing, non-coding RNA, nuclear receptor, nucleus and nucleolus, palmitoylation, pattern recognition receptors, peroxisomes, pro-apoptosis, pro-cell cycle, proteasome, pseudogenes, RAS superfamily, reactive oxygen species protection, secreted and extracellular matrix, transcription factors, transporters, transposon control, ubiquitylation and sumoylation, unfolded protein and stress, and unknown. Enrichment scores for each group are calculated based on an overlap p value to determine the functional groups over or under-expressed in the gene expression dataset. The BIG-C may be configured such that each gene is sorted into only one of the 53 functional groups, allowing for a quick and relatively simple understanding of types of genes enriched and co-expressed in a big dataset.

The I-Scope™ tool may be configured to identify immune infiltrates. Hematopoietic cells are unique in that they move throughout the body patrolling for threats to the host, and may infiltrate tissue sites not normally home to immune cells. I-Scope™ may be configured to identify hematopoietic cells through an iterative search of more than 17,000 genes identified in more than 50 microarray datasets. From this search, 1226 candidate genes are identified and researched for restriction in hematopoietic cells as determined by the HPA, GTEx and FANTOMS datasets (e.g., available at proteinatlas.org). 926 genes meet the criteria for being mainly restricted to hematopoietic lineages (brain, reproductive organ exclusions were permitted). These genes are researched for immune cell specific expression in 27 hematopoietic sub-categories: alpha beta T cell, T cell, regulatory T Cell, activated T cell, anergic T cell, gamma delta T cells, CD8 T, NK/NKT cell, NK cell, T & B cells, B cells, germinal center B cells, B cell and plasmacytoid dendritic cell, T &B & myeloid, B & myeloid, T & myeloid, MHC Class II expressing cell, monocyte, dendritic cell, plasmacytoid dendritic cells, myeloid cell, plasma cell, erythrocyte, neutrophil, low density granulocyte, granulocyte, and platelet. Transcripts are entered into I-Scope™ and the number of transcripts in each category determined. Odd's ratios are calculated with confidence intervals using the Fisher's exact test in R.

The T-Scope™ tool may be configured to help identify types of non-hematopoietic cells in gene expression datasets. T-Scope™ may be configured by downloading approximately 10,000 tissue enriched and 8,000 cell line enriched genes from the human protein atlas along with their tissue or cell line designation (e.g., available at proteinatlas.org). Genes found in more than four tissues are eliminated. Housekeeping genes described in the gene expression study by She et al. are also removed (e.g., as described by She et al., “Definition, conservation and epigenetics of housekeeping and tissue-enriched genes,” BMC Genomics 2009, 10:269, which is incorporated herein by reference in its entirety). This list is further curated by removing genes differentially expressed in 34 hematopoietic cell gene expression datasets and adding kidney specific genes from datasets downloaded from the GEO repository and processed by Ampel BioSolutions. The resulting categories of genes represent genes enriched in the following 42 tissue/cell specific categories: adrenal gland, breast, cartilage, cerebral cortex, uterine cervix, chondrocyte, colon, duodenum, endometrium, epididymis, esophagus fallopian tube, esophagus, fibroblast, heart muscle, keratinocyte, kidney, liver, lung, melanocyte, ovary pancreas, parathyroid gland, placenta, podocyte, prostrate, rectum, salivary gland, seminal vesicle, skeletal muscle, skin, small intestine, smooth muscle, stomach, synoviocyte, testis, kidney loop of henle, kidney proximal tubule, kidney distal tubule, and kidney collecting duct.

The CellScan tool may be a combination of I-Scope™ and T-Scope™, and may be configured to analyse tissues with suspected immune infiltrations that should also have tissue specific genes. CellScan may potentially be more stringent than either I-Scope™ or T-Scope™ because it may be used to distinguish resident tissue cells from non-resident hematopoietic cells.

The MS (Molecular Signature) Scoring tool may be configured to assess specific pathways in a disease state. Information on genes that encode for proteins that participate in a specific signaling pathway, and whether the gene product promotes or inhibits the pathway, are compiled and curated through literature mining. Curated pathways presented by the company include CD40-CD40ligand, IL-6, IL-12/23, TNF, IL-17, IL-21, S1P1, IL-13 and PDE4, but this method may be used for any known signaling pathway with available data. To determine if a signaling pathway is over or under-expressed in a microarray dataset, the gene list for each signaling pathway may be queried against the limma differentially expressed genes from a disease state compared to healthy controls, and the differentially expressed genes in the signaling pathway may be identified for each set. The fold changes for genes that promoted the pathway may be added together and the fold changes for genes that inhibited the pathway may be subtracted from the score. This total score may be normalized based on the number of genes that could be detected on the specific microarray platform used for the experiment. Activation scores of −100 to +100 may be determined using this method with negative scores indicating an inhibition of the specific pathway in the disease state and positive scores indicating an up-regulation of a specific pathway in the disease state. The Fischer's exact test may be performed to determine if there was sufficient overlap of genes between the experimental differentially expressed genes and the genes in the signaling pathway.

Gene Set Variation Analysis (GSVA) may be performed (for example, as described in Catalina et al. (2019, Communications Biology, “Gene expression analysis delineates the potential roles of multiple interferons in systemic lupus erythematosus”, which is incorporated herein by reference in its entirety) to determine enrichment of signaling pathways in individual patient samples. Gene set variation analysis may be performed using an open source software package for the coding language R available at the R Bioconductor (bioconductor.org), e.g., as described by Hanzelman et al., (“GSVA: gene set variation analysis for microarray and RNA-Seq data,” BMC Bioinformatics, 2013, which is incorporated herein by reference in its entirety). The modules of genes to interrogate the datasets may be developed. Modules of genes determined to represent a specific signaling pathway or process may be identified (e.g., using publicly available datasets). For example, the IFNB1 signaling pathway is taken from a publicly available gene expression dataset of peripheral blood cells treated with IFNB1 in vitro. Genes co-expressed in this dataset (genes either all increased or decreased compared to control treated peripheral blood) are used to create modules of genes representing the IFNB1 signaling pathway, and GSVA is used to determine the enrichment of this set of genes and hence the IFNB1 signaling pathway in individual patient and control samples.

The CoLTs®, or Combined Lupus Treatment Scoring, may be configured to rank identified drugs or therapies by a number of essential characteristics, including scientific rationale, experience in lupus mice/human cells (preclinical), previous clinical experience in autoimmunity, drug properties, and safety profile, including adverse events. Face and test validities may be established by scoring SOC medications and confirming the scores with a panel of lupus clinicians. The final result may be the CoLTs® score. A CoLTs® algorithm may also be configured for drugs in development (DID), which typically do not have drug metabolism and adverse event information available.

The target scoring algorithm may be configured to prioritize a specific gene or protein that is potentially a good choice to target with a drug in lupus patients. It may be utilized even if there is currently no drug available to the target gene or protein. The algorithm may be based on the addition of 18 data based determinations plus the overall scientific rationale and generates scores from −13 (not a good target in SLE) to 27 (very promising target in SLE).

BIG-C™ Big Data Analysis Tool

BIG-C® is a fast and efficient cloud-based tool to functionally categorize gene products. With coverage of over 80% of the genome, BIG-C® leverages publicly available databases such as UniProtKB/Swiss-Prot, GO terms, KEGG pathways, NCBI PubMed and Interactome to place genes into 53 functional categories. The sorting into only one of 53 functional groups allows for a quick and relatively simple understanding of types of genes enriched and co-expressed in a big dataset. This assists in deriving further insights from genes expressed for a given disease state in human or pre-clinical mouse models.

BIG-C® can be used to functionally categorize immunological genes that are not covered in cancer databases such as GO and KEGG (e.g., as described by Grammer et al. 2016, “Drug repositioning in SLE: crowd-sourcing, literature-mining and Big Data analysis,” Lupus, 25(10), 1150-1170, which is incorporated herein by reference in its entirety). Using a knowledge base of over 5000 patients with systemic lupus erythematosus (SLE), over 16432 genes are each placed into one of 53 BIG-C® functional categories, and statistical analysis is performed to identify enriched categories. BIG-C® categories are cross-examined with the GO and KEGG terms to obtain additional information and insights.

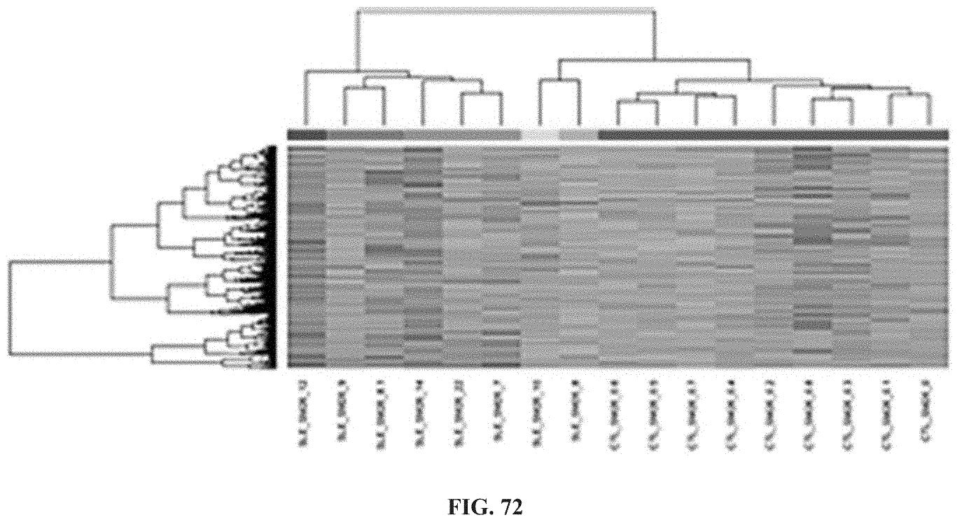

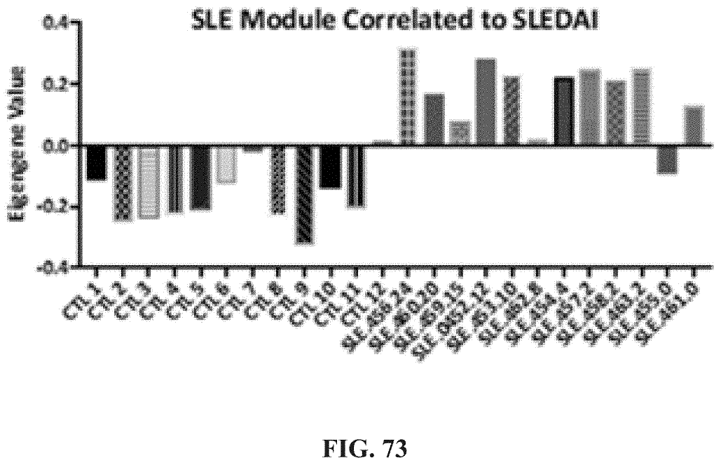

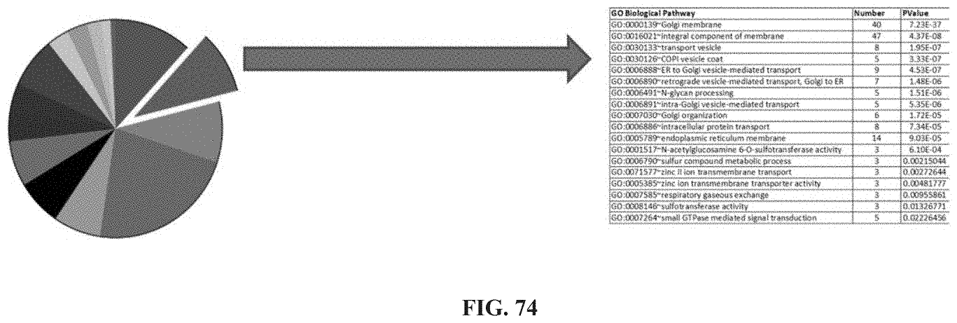

A sample BIG-C® workflow may comprise the following steps. First, SLE genomic datasets arederived from whole blood, peripheral blood mononuclear cells, affected tissues, and purified immune cells. Second, datasets are analyzed using DE analysis (as shown by differential expression heatmap in FIG. 72) or Weighted Gene Coexpression Network Analysis (WGCNA) (as shown by the gene coexpression plot in FIG. 73). Third, expressed genes are annotated using publicly available databases (e.g., UniProtKB/Swiss-Prot database, Human Immunodeficiencies database, Mouse MGI database, Entrez Molecular Sequence database, PubMed, and the Human Tissue Atlas). Fourth, signatures are cross-referenced with purified single-cell microarray datasets and RNAseq experiments. Fifth, BIG-C® is leveraged to separate the individual annotated genes into one of 53 functional categories shown in Table 50 (e.g., as described by Labonte et al. 2018, “Identification of alterations in macrophage activation associated with disease activity in systemic lupus erythematosus,” PloS one, 13(12), e0208132, which is incorporated herein by reference in its entirety). Sixth, chi-squared analysis is used to determine enriched categories of interest from overlap p-values. Seventh, enriched categories are cross-examined with GO and KEGG terms to derive key insights for further analysis (as shown by the enriched categories identified (left) and cross-referenced to GO terms (right) in FIG. 74).

TABLE 50
BIG-C Categories

| | | | | | | | | |
|---|---|---|---|---|---|---|---|---|
| Immune Cell | General Cell | Immune | Intracellular | MHC Class | MHC Class | Secreted | Secreted ECM | Pat. Recog. |
| Surface | Surface | Signaling | Signaling | I | II | Immune | | Receptors |
| Interferon | PRO-Cell | Anti-Cell | PRO | Anti | Unfold Prot. | Proteasome | Autophagy | Ubiquitylation |
| Gene Sig | Cycle | Cycle | Apoptosis | Apoptosis | Stress | | | |
| General | Transcript. | Nuc. Horm. | Chromatin | DNA | mRNA | mRNA | MicroRNA | Cytoskeleton |
| Transcript. | Factors | Receptors | Remodel | Repair | Translation | Splicing | Processing | |
| Integrin | RAS | WNT | Lysosome | Endocytosis | Endosome | Endoplas. | Oxidative | TCA Cycle |
| Pathway | Superfamily | Signaling | | | & Vesicles | Retic. | Phosphor. | |
| Mito. DNA | Mito | FA | Transporters | Cytoplasm | Peroxisomes | ROS | Nuclear & | Active RNA |
| to RNA | | Biosynth | | Biochem | | Protection | Nucleolus | |
| MicroRNA | Melanosome | Unknown | Pseudogenes | Transposon | Golgi | Glycolysis | Palmitoylation | |
| | | | | Control | | | | |

I-Scope™ Big Data Analysis Tool

I-Scope™ may be a tool configured for cross-examining the presence and activity of varying types of immune cell infiltrates with observed gene expression patterns. It may take annotated gene expression data and analyze it for hematopoietic cell lineage. I-Scope™ can be used downstream of the BIG-C® (Biologically Informed Gene-Clustering) tool in that it helps to provide even more insight into the nature of the genes being expressed after categorization.

I-Scope™ addresses the need to understand the involvement of specific cells for a given disease state. While it is helpful to understand the relative up-regulation and down-regulation at the gene expression level, it is even more informative to understand specifically in which cells this is occurring. I-Scope™ may be configured to identify hematopoietic cells through an iterative search of more than 17,000 genes identified in more than 50 microarray datasets (e.g., as described by Hubbard et al., “Analysis of Lupus Synovitis Gene Expression Reveals Dysregulation of Pathogenic Pathways Activated within Infiltrating Immune Cells,” Arthritis Rheumatol, 2018; 70 (suppl 10), which is incorporated herein by reference in its entirety). I-Scope™ may function by restricting the analysis to genes of hematopoietic cell heritage and allow for cross-checking against purified single-cell experiments or datasets. The cross-check confirms and categorizes specific transcript signatures to the 28 hematopoietic cell sub-categories shown in Table 51, ultimately allowing for cellular activity analysis across multiple samples and disease states. When combined with BIG-C® categories, the cellular activity can be correlated to specific functions within a given cell type.

TABLE 51
I-Scope ™ Cell Sub-Categories

| | | | | | | |
|---|---|---|---|---|---|---|
| Monos/Macs | Plasma | T-Cells | B-Cells | Dendritic | T&B Cells | CD8 T |
| | Cells | | | | | |
| Myeloid | Tact | LDG | Hematopoietic | Neutrophil | Ag | Granulocytes |
| Cells | | | | | Presentation | |
| Platelets | pDC | T, B, Mono | Langerhans | Bact | Mono and B | Erythrocytes |
| Mast | T reg | Gd T | T anergic | FDC | CD4T | T/NK/NKT |
| Cell | | | | | | Cells |

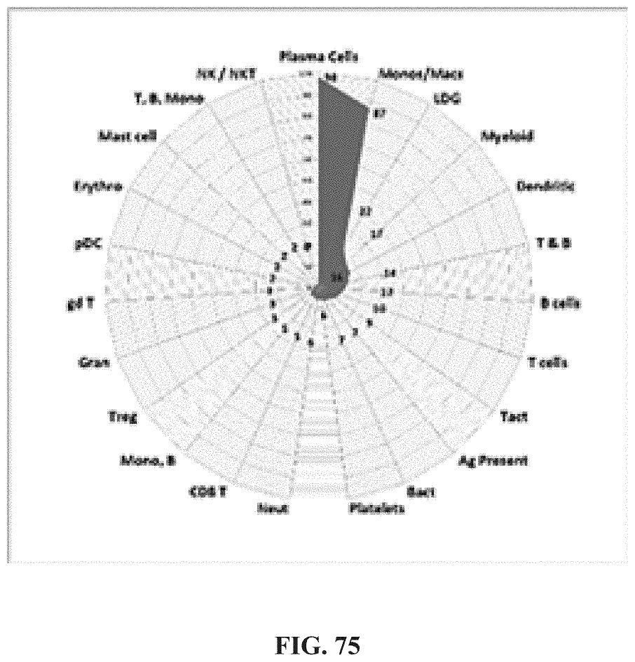

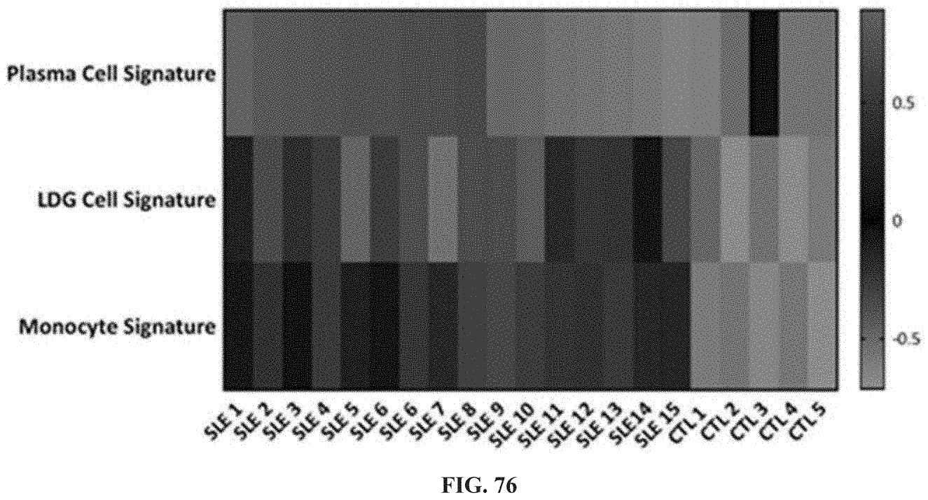

A sample I-Scope™ workflow may comprise the following steps. First, candidate genes are identified from SLE (systemic lupus erythematosus) datasets potentially associated with immune cell expression. Second, using HPA, GTEx, and FANTOMS datasets, expression signatures associated with hematopoietic cell lineage are identified. Third, signatures are cross-referenced with purified single-cell microarray datasets and RNAseq experiments. Fourth, transcripts are categorized into 28 hematopoietic cell sub-categories and assess cellular expression across different samples and disease states. Odd's ratios are calculated with confidence intervals using the Fisher's exact test in R. FIG. 75 shows an I-Scope™ signature analysis for a given sample, which leads to the I-Scope™ signature analysis across multiple samples and disease states (as shown in FIG. 76).

T-Scope™ Big Data Analysis Tool

The T-Scope™ tool may be configured for cross-examining gene expression signatures of a given sample with a database of non-hematopoietic cell types (e.g., as described by Hubbard et al., “Analysis of Gene Expression from Systemic Lupus Erythematosus Synovium Reveals Unique Pathogenic Mechanisms [Abstract], Annual Meeting of the American College of Rheumatology; June 2019; Chicago, Ill., which is incorporated herein by reference in its entirety). T-Scope™ may comprise a database of 704 transcripts allocated to 45 independent categories. Transcripts detected in the sample are matched to one of the cellular categories within the T-Scope™ tool to derive further insights on tissue cell activity. T-Scope™ can be used downstream of the BIG-C® (Biologically Informed Gene-Clustering) tool to understand which tissue cell types are present. In conjunction with I-Scope™ (which provides information related to immune cells), T-Scope™ can be performed to provide a complete view of all possible cell activity in a given sample.

T-Scope™ addresses the need to understand the involvement of specific tissue cells for a given disease state. While it is helpful to understand the relative up-regulation and down-regulation at the gene expression level, it is even more informative to understand specifically in which cells this is occurring. T-Scope™ may be configured by downloading a set of approximately 10,000 tissue enriched and 8,000 cell line enriched genes from the Human Protein Atlas along with their tissue or cell line designation. Genes differentially expressed in hematopoietic cell datasets are removed and kidney specific genes are added from the GEO repository. T-Scope™ may function by restricting the analysis to genes of known tissue cell heritage and allow for cross-checking against purified single-cell experiments or datasets. The cross-check confirms and categorizes specific transcript signatures to the 45 tissue cell sub-categories (as shown in Table 52), ultimately allowing for cellular activity analysis across multiple samples and disease states. When combined with BIG-C® categories, the cellular activity can be correlated to specific functions within a given tissue cell type.

TABLE 52
T-Scope ™ 45 Categories of Tissue Cells

| | | | | | | | | |
|---|---|---|---|---|---|---|---|---|
| Adipose | Adrenal | Breast | Cartilage | Cerebral | Cervix, | Chondrocyte | Colon | Dendritic |
| Tissue | Gland | | | Cortex | Uterine | | | |
| Duodenum | Endometrium | Endothelial | Epididymis | Erythrocytes | Esophagus | Fallopian | Fibroblast | Gallbaldder |
| | | | | | | Tube | | |
| Heart | Keratinocyte | Keratinocyte | Kidney | Kidney | Kidney | Kidney | Kidney | Kidney |
| Muscle | | Skin | | Distal | Loop | Proximal | Tubule | Tubule |
| | | | | Tubules | | Tubules | Duct | |
| Langherhans | Liver | Lung | Melanocyte | Podocyte | Prostate | Rectum | Salivary | Seminal |
| | | | | | | | Gland | Vesicle |
| Skeletal | Skin | Small | Smooth | Stomach | Synoviocyte | Testis | Thyroid | Urinary |
| Muscle | | Intenstine | Muscle | | | | Gland | Bladder |

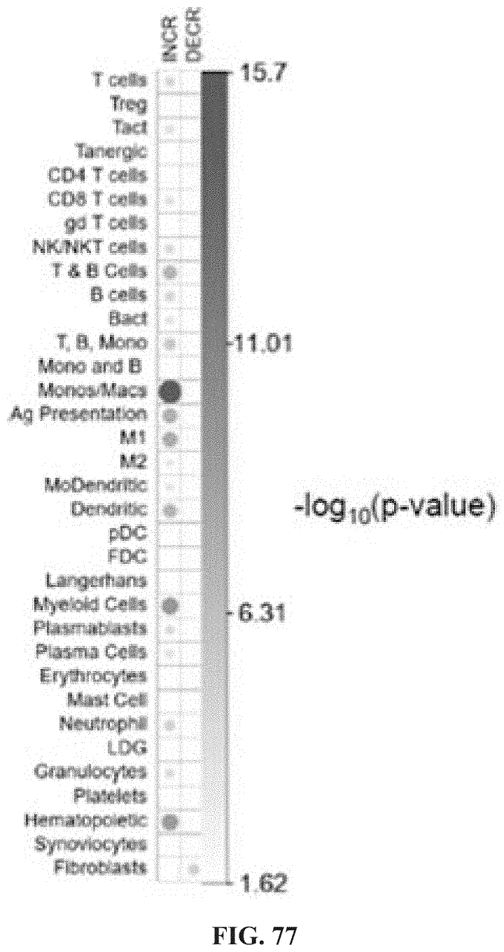

A sample T-Scope™ workflow may comprise the following steps. First, candidate genes are identified from SLE (systemic lupus erythematosus) differential expression datasets potentially associated with tissue cell expression. Second, using publicly available databases, expression signatures associated with potential tissue cell activity are identified. Third, signatures are cross-referenced with microarray, scRNAseq or RNAseq experiments. Fourth, transcripts are categorized into 45 tissue cell sub-categories and cellular expression is assessed across different samples and disease states. FIG. 77 shows results obtained using T-Scope™ in combination with I-Scope™ for identification of cells post-DE-analysis.

CellScan Big Data Analysis Tool

A cloud-based genomic platform may be configured to provide users with access to CellScan™, which comprises a suite of tools for the identification, analysis, and prioritization of targets for drug development and/or repositioning. This platform is powered by a database containing the genomic information gathered from 5000+ autoimmune patients. The cloud-based genomic platform may leverage results from RNAseq and microarray experiments in conjunction with clinical information, such as medication and lab tests, to provide previously undiscovered insights.

CellScan™ may go beyond typical ‘omics analysis by performing one or more of the following: functionally categorizing genes and their products (e.g., using BIG-C®); deconvolving gene expression data to identify unique immunological cell types from blood or biopsy samples (e.g., using I-Scope™); identifying tissue specific cell from biopsy samples (e.g., using T-Scope™); identifying receptor-ligand interactions and subsequent signaling pathways (e.g., using MS-Scoring™); ranking genes and their products for targeting by drugs and miRNA mimetics (e.g., using Target-Scoring™); and prioritizing FDA-approved drugs and drugs-in-development for treatment in patients or pre-clinical models (e.g., using CoLTs®).

CellScan™ applications may include one or more of: Biomarker Discovery, Disease Mechanisms, Drug Mechanism of Action, Drug Mechanism of Toxicity, and Target Identification and Validation. Experimental approaches supported by CellScan™ may include one or more of: lncRNA, Metabolomics, MicroArray, miRNA, mRNA, qPCR, Proteomics, and RNAseq.

Data analysis and interpretation with CellScan™ may build on comprehensive, manually curated content of a knowledge base. Powerful, quick, and efficient tools may be used to perform deep analysis of NGS and miRNA data to identify gene function, immunological and tissue cell type, pathways, and target/drug appropriate for a specific disease state.

CellScan™ features may be configured to optimize or maximize the impact of information that surfaces in an analysis so that interpretation of a dataset is comprehensive and elucidates actionable insights. These features may include one or more of: NGS RNAseq data analysis, biomarker scoring, and prioritizing targets and drugs for human clinical trials and/or pre-clinical models. The NGS RNAseq data analysis may comprise interrogating RNA and miRNA data for function, cell-type (immunological or tissue) and pathways. The biomarker scoring may comprise using a knowledge base and gene expression data to assess and prioritize biomarkers associated with a target disease or phenotype. The target/drug prioritization may comprise leveraging objective scoring of targets and drugs based on parameters such as scientific rationale, evidence in mouse/human cells, prior clinical data, overall drug properties, and the risk of adverse events.

The knowledge base may be a repository created from millions of individual pieces of information gathered about genes, cells, tissues, drugs, and diseases, and manually reviewed for accuracy and includes rich contextual details and links to original publications. The knowledge base may enable access to relevant and substantiated knowledge from primary literature as well as public and private databases for comprehensive interpretation of NGS/RNAseq data elucidating function/pathways and prioritize targets/drugs for given disease states. Table 53 shows an example list of reference databases for the content in CellScan™, with both human and mouse species-specific identifiers supported.

TABLE 53
Reference Databases for Content in CellScan ™

| | | | |
|---|---|---|---|
| Affymetrix | Entrez Gene | HPA | scRNAseq |
| Agilent | FANTOM5 | Illumina | STITCH |
| BrainArray | GenBank | Interactome | Mouse Genome |
| | | | Database (MGD) |
| CAS Registry Number | Gene Symbol - human | KEGG | UCSC (hg18) |
| | (Hugo/HGNC) | | |
| Clinicaltrials.gov | Gene Symbol - mouse | LINCS/CLUE | UCSC (hg19) |
| | (Entrez Gene) | | |
| CodeLink | GNF Tissue Expression | Mosby's Drug | Unigene |
| | Body Atlas | Consult | |
| DrugBank | GO terms | NCBI PubMed | Uniprot/Swiss- |
| | | | Prot Accession |
| Drugs@FDA | Goodman & Gilman's | NCI-60 Cell Line | |
| | Pharmacological | Expression Atlas | |
| | Basis of Therapeutics | | |
| Ensembl | GTEx | Refseq | |

MS (Molecular Signature) Scoring™ Analysis Tool

MS-Scoring™ may be configured to identify receptor-ligand interactions and predict ongoing signaling pathways. In addition, MS-Scoring™ may be used to validate molecular pathways as potential targets for new or repurposed drug therapies. The specificity of next-generation drug therapies requires a way to understand the potential of a given therapy to act on the intended biochemical target. Moreover, a potential application of this is the repositioning of drug therapies that may have the correct biochemical targeting to address multiple clinical needs beyond the initial intended therapeutic value.

MS-Scoring™ may be specifically developed to address gaps in the QIAGEN IPA® (Ingenuity Pathway Analysis) tool that does not contain many immunologically relevant pathways. Similar to IPA®, MS-Scoring™ 1 may use log-fold change information to score the target and its signaling pathway to verify the viability of the targets. If the fold-change of the genes of a signaling pathway appears to be upregulated or inhibitors appear to be downregulated, MS-Scoring™ 1 may provide a score of +1. Conversely if the genes of a signaling pathway appear downregulated or the inhibitors upregulated, MS-Scoring™ 1 may provide a score of −1. A score of zero may be provided if no fold-change is observed. The scores may then be summed and normalized across the entire pathway to yield a final % score between −100 (inhibition) and +100 (up-regulation). Higher absolute magnitude scores, scores that are close to −100 or +100, may indicate a high potential for therapeutic targeting. The Fischer's exact test may be performed to determine if there is sufficient overlap of genes between the experimental differentially expressed genes and the genes in the signaling pathway.

A sample MS-Scoring™ 1 workflow may comprise the following steps. First, potential drugs and pathways are identified by LINCS (Library of Integrated Network-Based Cellular Signatures) as candidates for therapeutic intervention. Second, MS-Scoring™ 1 is used to evaluate individual transcript elements of the target pathway. Third, signatures are cross-referenced with purified single-cell microarray datasets and RNAseq experiments. Fourth, scores are compiled and normalized to provide an overall % score for the pathway and higher absolute magnitude scores indicate a higher potential for therapeutic targeting.

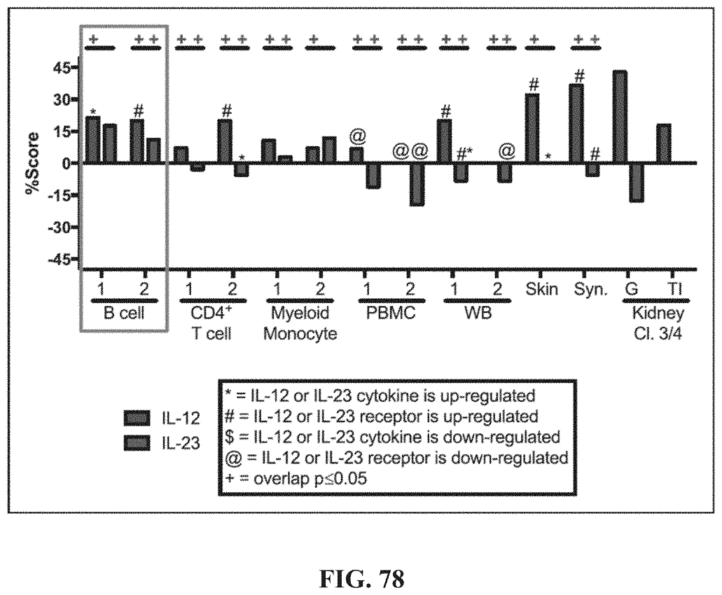

FIG. 78 shows MS-Scoring™ 1 of IL-12 and IL-23 related pathways for targeting using ustekinumab for SLE (systemic lupus erythematosus) drug repositioning (e.g., as described by Grammer et al., 2016, “Drug repositioning in SLE: crowd-sourcing, literature-mining and Big Data analysis,” Lupus, 25(10), 1150-1170, which is incorporated herein by reference in its entirety).

MS-Scoring™ 2 may utilize custom-defined gene modules that represent a signaling pathway or process and is particularly useful for gene expression datasets from microarray or RNAseq. The MS-Scoring™ 2 tool may be configured to take a deeper look at signaling pathways analyzed using the MS-Scoring™ 1. The tool may analyze raw gene expression data and assess enrichment by the Gene Set Variation Analysis (as described herein), which assigns an indexed score to the individual co-expressed pathways between −1 and +1 indicating levels of down-regulation and up-regulation respectively.

A sample MS-Scoring™ 2 workflow may comprise the following steps. First, a signaling pathway of interest is selected from the MS-Scoring™ 2 menu. Second, a raw gene expression data is inputted into the MS-Scoring™ 2 tool. Third, enrichment of signaling pathway(s) is assessed on a patient by patient basis. Fourth, the data can then be used to drive insight for the target signaling pathways in individual patient samples.

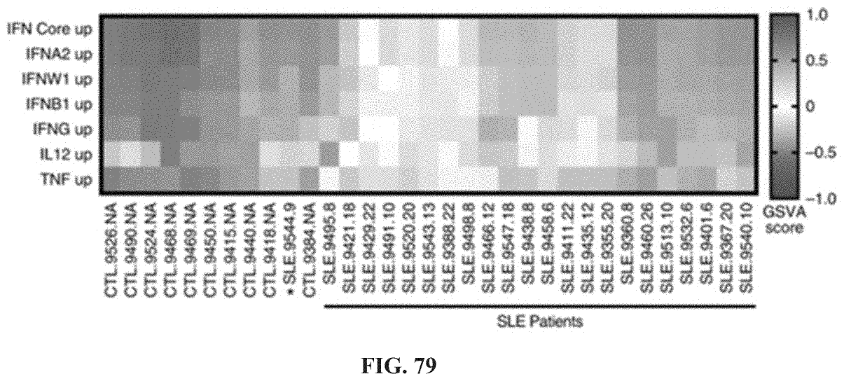

FIG. 79 shows results from GSVA Analysis on SLE (systemic lupus erythematosus) signaling pathways, e.g., as described by Hänzelmann et al., “GSVA: Gene Set Variation Analysis for Microarray and RNA-Seq Data,” BMC Bioinformatics, vol. 14, no. 1, 2013, p. 7., which is incorporated herein by reference in its entirety.

CoLTs® (Combined Lupus Treatment Scoring) Analysis Tool

A scoring method called CoLTs®, or Combined Lupus Treatment Scoring, may be configured to assessing and prioritizing the repositioning potential of drug therapies. CoLTs® may rank identified drugs/therapies by a number of essential characteristics, including scientific rationale, experience in lupus mice/human cells (preclinical), previous clinical experience in autoimmunity, drug properties, and safety profile, including adverse events. Face and test validities may be established by scoring standard of care (SOC) medications and confirming the scores with a panel of lupus clinicians. The final result may be the CoLTs® score. A CoLTs® algorithm may also be configured for drugs in development (DID) since they typically do not have drug metabolism and adverse event information available. The algorithms for CoLTs® scoring are shown in Table 54.

TABLE 54
Algorithms for CoLTs ® Scoring

| | CoLTs FDA- | | |
|---|---|---|---|
| | Approved | | CoLTs DID |
| | Algorithm | | Algorithm |
| Score Category | Points | Question | Points |
| Rationale | 0 to +3 | Does the mechanism have a role in lupus pathogenesis? (0) No Role in lupus, (+1) possible role | 0 to +3 |
| | | in lupus, (+2) likely role in lupus, (+3) demonstrated role in lupus | |
| Lupus Mice | −1 to +1 | Has the drug been used to treat lupus in mice? (−1) no benefit, (0) not tried/conflicting results, | −1 to +1 |
| | | (+1) efficacious in lupus mice | |
| Lupus Cells in | −1 to +1 | Has the drug been used in in vitro experiments with human cells? (−1) no benefit, (0) not | −1 to +1 |
| vitro | | studied/conflicting results, (+1) reduced lupus abnormalities in vitro with lupus derived cells | |
| Lupus | −1 to +1 | Is the target of the drug abnormal in lupus? (−1) studied but not present, (0) not | −1 to +1 |
| Abnormalities | | studied/conflicting results, (+1) drug target is active and/or present in lupus | |
| Drug Clinical | −1 to +1 | Has the drug been used to treat autoimmune disease? (−1) tried but not benefit, (0) not tried, (+1) | −1 to +1 |
| Experience in | | trial or case report with benefit | |
| Autoimmunity | | | |
| Drug Clinical | −1 to +1 | Has the drug been used to treat lupus? (−1) Tried but no benefit - failed primary endpoint in | −1 to +1 |
| Experience in | | Phase 2b, (0) not tried/ongoing/failed primary Phase 2b endpoint with some positive result, (+1) | |
| Lupus | | trial with benefit in Phase 2b clinical trials | |
| Drug Properties | −3 to +3 | Does the drug interact with current SLE drugs? (−1) if it interacts with corticosteroids, NSAIDs, | N/A |
| | | MMF, MTX, AZA, statins, chloroquines, cyclophosphamide, ACE inhibitors). Is binding | |
| | | reversible? (−1) covalent inhibition, (0) noncovalent inhibition. How the drug is administered? | |
| | | (+1) SC, (0) IV. How frequently is the drug administered? (1) by mouth once daily, (0) more than | |
| | | one time per day. Is the drug a human/humanized antibody? (+1) human/humanized, (0) | |
| | | not/chimeric. Is this drug specific? (+1) one target/specific, (0) effective but not targeted, i.e. | |
| | | downstream, (−1) many targets/nonspecific | |
| Induces Lupus | −1 to 0 | Does the drug induce lupus? (−1) Induces lupus, no reports of drug induced lupus (0) | N/A |
| Drug Metabolism | −2 to 0 | Is the drug metabolized using p450 and/or through the kidneys? (−2) If p450 and kidney excretion | N?A |
| | | >20%, (−1) If p450 issues or kidney excretion >20%, (0) If neither | |
| Adverse Events | −5 to 0 | Reported adverse events and Black Box Warnings from Medscape and DailyMed for each for | NA |
| | | drug are compared to the 150 scored adverse events (each event is scored from −1 to −5 based | |
| | | upon severity). The individual adverse events scores for each drug are summed to create the tox | |
| | | score, which is multiplied by the number of adverse events to create the tox product. Then tox | |
| | | product is then normalized to produce a score ranging from −5 to 0 | |
| Range | −16 to 11 | | −5 to +8 |

CoLTs® may be configured to perform objective scoring of drug molecules based on a hypothesis-based literature search of publicly available databases. The tool has the ability to rank drug molecules from both FDA-approved and non-approved classes and ranked based upon parameters such as scientific rationale, evidence in mouse/human cells, prior clinical data, overall drug properties, and the risk of adverse events. The parameters are used within five independent drug therapy categories: small molecules, biologics, complementary and alternative therapies, and drugs in development.

CoLTs® may address the need for a systematic and objective way to evaluate the potential of drug therapies to be repositioned for treatment of autoimmune diseases, initially within SLE (systemic lupus erythematosus). The composite score may embody all the accessible information in literature databases, inclusive of efficacy and adverse reactions, to be able to assist in the prioritization of drug development. While the composite score takes into account many aspects of a drug, it may heavily weigh the risk of adverse events and ranges from −16 to +11. CoLT Scoring® may be validated through repeated scoring of 215 potential therapies using a total of over 5000 reference data points as well as by clinicians specializing in the field of rheumatology. Specifically, CoLTs®' prediction of Stelara/Ustekinumab to be a top priority biologic for lupus drug repositioning is validated by a successful Phase 2 clinical trial (e.g., as described by Vollenhoven et al., “Efficacy and Safety of Ustekinumab, an IL-12 and IL-23 Inhibitor, in Patients with Active Systemic Lupus Erythematosus: Results of a Multicentre, Double-Blind, Phase 2, Randomised, Controlled Study.” The Lancet, vol. 392, no. 10155,2018, pp. 1330-1339, which is incorporated herein by reference in its entirety). CoLTs® may be calibrated on SoC (Standard of Care) therapies for the individual autoimmune disease being assessed.

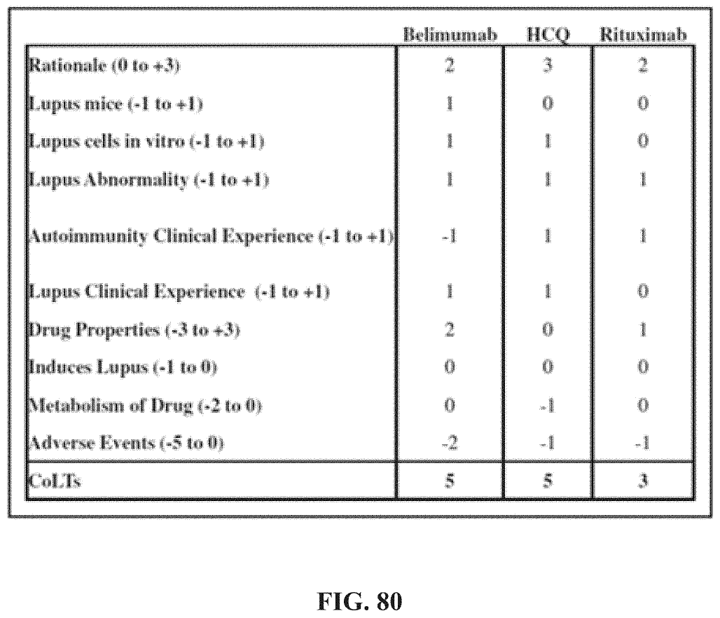

Within the ten major categories, rationale ranges from 0 to +3, mouse/human in vitro experience ranges from −1 to +1, clinical properties are on a scale of −3 to +3, the adverse effect of inducing lupus ranges from −1 to 0, metabolic properties range from −2 to 0, and finally adverse events (such as toxicity, infection, carcinogenic, etc.) were given a score of −5 to 0 (e.g., as described by Grammer et al., 2016, “Drug repositioning in SLE: crowd-sourcing, literature-mining and Big Data analysis,” Lupus, 25(10), 1150-1170, which is incorporated herein by reference in its entirety). FIG. 80 shows the CoLT Scoring® of SOC Therapies in Lupus (Belimumab, HCQ, and Rituximab).

Target Scoring Analysis Tool

The Target scoring algorithm may be configured to prioritize a specific gene or protein that would potentially be a good choice to target with a drug in lupus patients. It may be utilized even if there is currently no drug available to the target gene or protein. The algorithm may be based on the addition of 18 data based determinations plus the overall scientific rationale and generates scores from −13 (not a good target in SLE) to 27 (very promising target in SLE). The scoring system is shown in Table 55.

TABLE 55
Target Scoring Algorithm

| Scoring Category | Points | Question |
|---|---|---|
| Genetically Alt Mice | −1 to 3 | Has the gene been studied in genetically altered mice (−1 to 3) 9-1 not viable, 0 no mouse; +1 |
| | | immunological phenotype, +2 immunological phenotype with autoimmunity, +3 immunological |
| | | phenotype w lupus) |
| Human Deletion | 0 to 2 | Is the gen associated with a human genetic deficiency? (0 to 2) (0 none, +2 |
| | | immunological/inflammatory/immunodeficiency disease) |
| Lupus Mouse Express | −1 to 1 | Do lupus mice have mRNA or Protein expression (−1 to +1) |
| Gene Cross Lupus Mice | −1 to 1 | Lupus mice genetic (cross into lupus strain) (−1 to +1): no known genetic component or makes lupus |
| | | mouse worse (−1); 0, no involvement or no impact) to known genetic component or genetic |
| | | manipulation makes lupus mouse better (+1) |
| Assoc Func PW in Mice | −1 to 1 | Does the gen associate with a functional pathway known to be abnormal in lupus mice? (−1 to +1) |
| Assoc Func PW in Humans | −1 to 1 | Does the gene associate with a functional pathway known to be abnormal in human SLE? (−1 to +1) |
| GWAS | 0 to 1 | Identified as associated with lupus by GWAS or deep sequencing: (0 = no, 1 = yes) |
| Gene Methylation | −1 to 1 | Identified as associated with lupus by Methylation Data (0 = no, 1 = yes) |
| In vitro data | −1 to 1 | Gene is implicated in in vitro experiments upus cells in vitro (−1 to +1) |
| Change after human SOC | −1 to 1 | Protein or mRNA (or pathway) changed in lupus mouse by treating w a drug (−1 to +1)? |
| Drug target in lupus mouse | −1 to 1 | Protein or mRNA (or pathway) changed in lupus mouse by treating w a drug (−1 to +1)? |
| CLUE | −1 to 1 | Does CLUE analysis support the pathway as potentially involved in lupus (−1 to +1)? |
| Lupus Biomarker | 0 to 1 | Can the target be used as a biomarker in lupus? (0 = no, 1 = yes) |
| Redundancy | −3 to 1 | Is the target non-redundant, no multiple ligand receptor interactions? −3 to 1 |
| WGCNA | 0 to 3 | Is the gene associated with on disease parameter (+1), two (+2), or three (+3) |
| Tissue Consensus | 0 to 2 | Is the gene Overexpressed in SLE tissues? 0 for none, 1 for 1 tissue, 2 for two or more SLE tissues |
| Upstream Regulator | 0 to 1 | Is the gene an UPR in IPA with significant z-score (>3)? 0 = no, +1 = yes |
| Hematopoietic Restricted | 0 to 1 | Is the gene hematopoetically restricted? 0 = no, +1 = yes |
| Biologic Rationale | 0 to 3 | Rationale/Mechanism: no role (or no information) to demonstrated role in lupus pathogenesis (0 to |
| | | +3) |
| Target Data Score | −13 to 27 | |

Target-Scoring™ may be configured to assessing and prioritizing the potential of molecular targets for further development of drug therapies. The Target-Scoring™ tool is very similar to CoLTs® except it approaches the need for new SLE therapies from a different angle. Target Scoring may be configured to perform an objective assessment of molecular targets for the development of new or repurposed drug therapies. Like CoLTs®, it also derives data from a hypothesis-based literature search and generates a composite score based on the publicly available information. Leveraging the composite score, researchers can better prioritize the development of novel drug therapies addressing the assessed targets of interest.

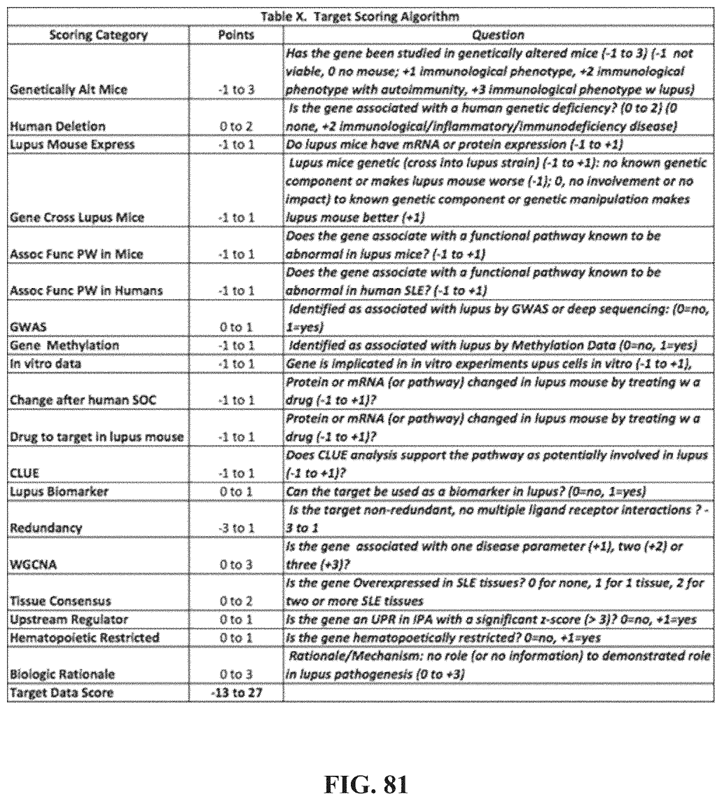

Target-Scoring™ may utilize 19 different scoring categories (as shown by the Target-Scoring categories and point values in FIG. 81) to derive a composite score that ranges from −13 to +27 for the suitability of a gene target for SLE therapy development. Target-Scoring™ may be validated through repeated scoring of potential therapies as well as by clinicians (e.g., clinicians specializing in the field of immunology).

Classifiers

In some embodiments, the present disclosure provides a system, method, or kit having data analysis realized in software application, computing hardware, or both. In various embodiments, the analysis application or system includes at least a data receiving module, a data pre-processing module, a data analysis module, a data interpretation module, or a data visualization module. In one embodiment, the data receiving module can comprise computer systems that connect laboratory hardware or instrumentation with computer systems that process laboratory data. In one embodiment, the data pre-processing module can comprise hardware systems or computer software that performs operations on the data in preparation for analysis. Examples of operations that can be applied to the data in the pre-processing module include affine transformations, denoising operations, data cleaning, reformatting, or subsampling. A data analysis module, which can be specialized for analyzing genomic data from one or more genomic materials, can, for example, take assembled genomic sequences and perform probabilistic and statistical analysis to identify abnormal patterns related to a disease, pathology, state, risk, condition, or phenotype. A data interpretation module can use analysis methods, for example, drawn from statistics, mathematics, or biology, to support understanding of the relation between the identified abnormal patterns and health conditions, functional states, prognoses, or risks. A data visualization module can use methods of mathematical modeling, computer graphics, or rendering to create visual representations of data that can facilitate the understanding or interpretation of results.

Feature sets may be generated from datasets obtained using one or more assays of a biological sample obtained or derived from a subject, and a trained algorithm may be used to process one or more of the feature sets to identify or assess a condition (e.g., a disease or disorder, such as a lupus condition) of a subject. For example, the trained algorithm may be used to apply a machine learning classifier to a plurality of condition-associated genomic loci that are associated with two or more classes of individuals inputted into a machine learning model, in order to classify a subject into one of the two or more classes of individuals. For example, the trained algorithm may be used to apply a machine learning classifier to a plurality of condition-associated that are associated with individuals with known conditions (e.g., a disease or disorder, such as a lupus condition) and individuals not having the condition (e.g., healthy individuals, or individuals who do not have a lupus condition), in order to classify a subject as having the condition (e.g., positive test outcome) or not having the condition (e.g., negative test outcome).

The trained algorithm may be configured to identify the presence (e.g., positive test result) or absence (e.g., negative test result) of one or more conditions (e.g., a disease or disorder, such as a lupus condition) with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99%. This accuracy may be achieved for a set of at least about 25, at least about 50, at least about 100, at least about 150, at least about 200, at least about 250, at least about 300, at least about 350, at least about 400, at least about 450, at least about 500, at least about 1,000, or more than about 1,000 independent samples.

The trained algorithm may comprise a machine learning algorithm, such as a supervised machine learning algorithm. The supervised machine learning algorithm may comprise, for example, a Random Forest, a support vector machine (SVM), a neural network, or a deep learning algorithm. The trained algorithm may comprise a classification and regression tree (CART) algorithm. The trained algorithm may comprise an unsupervised machine learning algorithm.

The trained algorithm may comprise a classifier configured to accept as input a plurality of input variables or features (e.g., condition-associated genomic loci) and to produce or output one or more output values based on the plurality of input variables or features (e.g., condition-associated genomic loci). The plurality of input variables or features may comprise one or more datasets indicative of the presence (e.g., positive test result) or absence (e.g., negative test result) of one or more conditions (e.g., a disease or disorder, such as a lupus condition). For example, an input variable or feature may comprise a number of sequences corresponding to or aligning to each of the plurality of condition-associated genomic loci.

The plurality of input variables or features may also include clinical information of a subject, such as health data. For example, the health data of a subject may comprise one or more of: a diagnosis of one or more conditions (e.g., a disease or disorder, such as a lupus condition), a prognosis of one or more conditions (e.g., a disease or disorder, such as a lupus condition), a risk of having one or more conditions (e.g., a disease or disorder, such as a lupus condition), a treatment history of one or more conditions (e.g., a disease or disorder, such as a lupus condition), a history of previous treatment for one or more conditions (e.g., a disease or disorder, such as a lupus condition), a history of prescribed medications, a history of prescribed medical devices, age, height, weight, sex, smoking status, and one or more symptoms of the subject.

For example, the disease or disorder may comprise one or more of: systemic lupus erythematosus (SLE), discoid lupus erythematosus (DLE), and lupus nephritis (LN). As another example, the symptoms may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof. As another example, the prescribed medications or drugs may include one or more of: antimalarials, corticosteroids, immunosuppressants, and nonsteroidal anti-inflammatory drugs (NSAIDs).

The trained algorithm may comprise a classifier, such that each of the one or more output values comprises one of a fixed number of possible values (e.g., a linear classifier, a logistic regression classifier, etc.) indicating a classification of the sample by the classifier. The trained algorithm may comprise a binary classifier, such that each of the one or more output values comprises one of two values (e.g., {0, 1}, {positive, negative}, or {high-risk, low-risk}) indicating a classification of the sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the sample by the classifier.

The classifier may be configured to classify samples by assigning output values, which may comprise descriptive labels, numerical values, or a combination thereof. Some of the output values may comprise descriptive labels. Such descriptive labels may provide an identification or indication of the presence (e.g., positive test result) or absence (e.g., negative test result) of one or more conditions (e.g., a disease or disorder, such as a lupus condition) of the subject, and may comprise, for example, positive, negative, high-risk, intermediate-risk, low-risk, or indeterminate. Such descriptive labels may provide an identification of a treatment for the one or more conditions of the subject, and may comprise, for example, a therapeutic intervention, a duration of the therapeutic intervention, and/or a dosage of the therapeutic intervention suitable to treat the one or more conditions of the subject. Such descriptive labels may provide an identification of secondary clinical tests that may be appropriate to perform on the subject, and may comprise, for example, an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof. For example, such descriptive labels may provide a prognosis of the one or more conditions of the subject. As another example, such descriptive labels may provide a relative assessment of the one or more conditions of the subject. Some descriptive labels may be mapped to numerical values, for example, by mapping “positive” to 1 and “negative” to 0.

The classifier may be configured to classify samples by assigning output values that comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the one or more conditions (e.g., a disease or disorder, such as a lupus condition) of the subject. Some numerical values may be mapped to descriptive labels, for example, by mapping 1 to “positive” and 0 to “negative.”

The classifier may be configured to classify samples by assigning output values based on one or more cutoff values. For example, a binary classification of samples may assign an output value of “positive” or 1 if the sample indicates that the subject has at least a 50% probability of having one or more conditions (e.g., a disease or disorder, such as a lupus condition), thereby assigning the subject to a class of individuals receiving a positive test result. As another example, a binary classification of samples may assign an output value of “negative” or 0 if the sample indicates that the subject has less than a 50% probability of having one or more conditions (e.g., a disease or disorder), thereby assigning the subject to a class of individuals receiving a negative test result. In this case, a single cutoff value of 50% is used to classify samples into one of the two possible binary output values or classes of individuals (e.g., those receiving a positive test result and those receiving a negative test result). Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, the classifier may be configured to classify samples by assigning an output value of “positive” or 1 if the sample indicates that the subject has a probability of having one or more conditions (e.g., a disease or disorder, such as a lupus condition) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of having one or more conditions (e.g., a disease or disorder, such as a lupus condition) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classifier may be configured to classify samples by assigning an output value of “negative” or 0 if the sample indicates that the subject has a probability of having one or more conditions (e.g., a disease or disorder, such as a lupus condition) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having one or more conditions (e.g., a disease or disorder, such as a lupus condition) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classifier may be configured to classify samples by assigning an output value of “indeterminate” or 2 if the sample is not classified as “positive”, “negative”, 1, or 0. In this case, a set of two cutoff values is used to classify samples into one of the three possible output values or classes of individuals (e.g., corresponding to outcome groups of individuals having “low risk,” “intermediate risk,” and “high risk” of having one or more conditions, such as a disease or disorder). Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values or classes of individuals, where n is any positive integer.

The trained algorithm may be trained with a plurality of independent training samples. Each of the independent training samples may comprise a sample from a subject, associated datasets obtained by assaying the sample (as described elsewhere herein), and one or more known output values or classes of individuals corresponding to the sample (e.g., a clinical diagnosis, prognosis, absence, or treatment efficacy of a condition of the subject). Independent training samples may comprise samples and associated datasets and outputs obtained or derived from a plurality of different subjects. Independent training samples may comprise samples and associated datasets and outputs obtained at a plurality of different time points from the same subject (e.g., on a regular basis such as weekly, biweekly, or monthly), as part of a longitudinal monitoring of a subject before, during, and after a course of treatment for one or more conditions of the subject. Independent training samples may be associated with presence of the condition (e.g., training samples comprising samples and associated datasets and outputs obtained or derived from a plurality of subjects known to have the condition). Independent training samples may be associated with absence of the condition (e.g., training samples comprising samples and associated datasets and outputs obtained or derived from a plurality of subjects who are known to not have a previous diagnosis of the condition or who have received a negative test result for the condition).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise samples associated with presence of the condition and/or samples associated with absence of the condition. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the condition (e.g., a disease or disorder, such as a lupus condition). The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with absence of the condition (e.g., a disease or disorder, such as a lupus condition). In some embodiments, the sample is independent of samples used to train the trained algorithm.

The trained algorithm may be trained with a first number of independent training samples associated with a presence of the condition (e.g., a disease or disorder, such as a lupus condition) and a second number of independent training samples associated with an absence of the condition (e.g., a disease or disorder, such as a lupus condition). The first number of independent training samples associated with presence of the condition (e.g., a disease or disorder, such as a lupus condition) may be no more than the second number of independent training samples associated with absence of the condition (e.g., a disease or disorder, such as a lupus condition). The first number of independent training samples associated with a presence of the condition (e.g., a disease or disorder) may be equal to the second number of independent training samples associated with an absence of the condition (e.g., a disease or disorder, such as a lupus condition). The first number of independent training samples associated with a presence of the condition (e.g., a disease or disorder, such as a lupus condition) may be greater than the second number of independent training samples associated with an absence of the condition (e.g., a disease or disorder, such as a lupus condition).

The trained algorithm may comprise a classifier configured to identify the presence (e.g., positive test result) or absence (e.g., negative test result) of one or more conditions (e.g., a disease or disorder, such as a lupus condition) at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the presence (e.g., positive test result) or absence (e.g., negative test result) of the one or more conditions by the trained algorithm may be calculated as the percentage of independent test samples (e.g., subjects known to have the condition or subjects with negative clinical test results for the condition) that are correctly identified or classified as having or not having the condition.

The trained algorithm may comprise a classifier configured to identify one or more conditions (e.g., a disease or disorder, such as a lupus condition) with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the condition using the trained algorithm may be calculated as the percentage of samples identified or classified as having the condition that correspond to subjects that truly have the condition.

The trained algorithm may comprise a classifier configured to identify one or more conditions (e.g., a disease or disorder, such as a lupus condition) with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the condition using the trained algorithm may be calculated as the percentage of samples identified or classified as not having the condition that correspond to subjects that truly do not have the condition.

The trained algorithm may comprise a classifier configured to identify one or more conditions (e.g., a disease or disorder, such as a lupus condition) with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the condition using the trained algorithm may be calculated as the percentage of independent test samples associated with presence of the condition (e.g., subjects known to have the condition) that are correctly identified or classified as having the condition.

The trained algorithm may comprise a classifier configured to identify one or more conditions (e.g., a disease or disorder, such as a lupus condition) with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the condition using the trained algorithm may be calculated as the percentage of independent test samples associated with absence of the condition (e.g., subjects with negative clinical test results for the condition) that are correctly identified or classified as not having the condition.

The trained algorithm may comprise a classifier configured to identify the presence (e.g., positive test result) or absence (e.g., negative test result) of one or more conditions (e.g., a disease or disorder, such as a lupus condition) with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying samples as having or not having the condition.

Classifiers of the trained algorithm may be adjusted or tuned to improve or optimize one or more performance metrics, such as accuracy, PPV, NPV, clinical sensitivity, clinical specificity, AUC, or a combination thereof (e.g., a performance index incorporating a plurality of such performance metrics, such as by calculating a weight sum therefrom), of identifying the presence (e.g., positive test result) or absence (e.g., negative test result) of the condition. The classifiers may be adjusted or tuned by adjusting parameters of the classifiers (e.g., a set of cutoff values used to classify a sample as described elsewhere herein, or weights of a neural network) to improve or optimize the performance metrics. The one or more classifiers may be adjusted or tuned so as to reduce an overall classification error (e.g., an “out-of-bag” or oob error rate for a Random Forest classifier). The one or more classifiers may be adjusted or tuned continuously during the training process (e.g., as sample datasets are added to the training set) or after the training process has completed.

The trained algorithm may comprise a plurality of classifiers (e.g., an ensemble) such that the plurality of classifications or outcome values of the plurality of classifiers may be combined to produce a single classification or outcome value for the sample. For example, a sum or a weighted sum of the plurality of classifications or outcome values of the plurality of classifiers may be calculated to produce a single classification or outcome value for the sample. As another example, a majority vote of the plurality of classifications or outcome values of the plurality of classifiers may be identified to produce a single classification or outcome value for the sample. In this manner, a single classification or outcome value may be produced for the sample having greater confidence or statistical significance than the individual classifications or outcome values produced by each of the plurality of classifiers.

After the trained algorithm is initially trained, a subset of the inputs may be identified as most influential or most important to be included for making high-quality classifications (e.g., having highest permutation feature importance). For example, a subset of the panel of condition-associated genomic loci may be identified as most influential or most important to be included for making high-quality classifications or identifications of conditions (or sub-types of conditions). The panel of condition-associated genomic loci, or a subset thereof, may be ranked based on classification metrics indicative of each influence or importance of each individual condition-associated genomic locus toward making high-quality classifications or identifications of conditions (or sub-types of conditions). Such metrics may be used to reduce, in some cases significantly, the number of input variables (e.g., predictor variables) that may be used to train the one or more classifiers of the trained algorithm to a desired performance level (e.g., based on a desired minimum accuracy, PPV, NPV, clinical sensitivity, clinical specificity, AUC, or a combination thereof).

For example, if training a classifier of the trained algorithm with a plurality comprising several dozen or hundreds of input variables to the classifier results in an accuracy of classification of more than 99%, then training the classifier of the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

As another example, if training a classifier of the trained algorithm with a plurality comprising several dozen or hundreds of input variables to the classifier results in a sensitivity or specificity of classification of more than 99%, then training the classifier of the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable sensitivity or specificity of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

The subset of the plurality of input variables (e.g., the panel of condition-associated genomic loci) to the classifier of the trained algorithm may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics (e.g., permutation feature importance).

Upon identifying the subject as having one or more conditions (e.g., a disease or disorder, such as a lupus condition), the subject may be optionally provided with a therapeutic intervention (e.g., prescribing an appropriate course of treatment to treat the one or more conditions of the subject). The therapeutic intervention may comprise a prescription of an effective dose of a drug, a further testing or evaluation of the condition, a further monitoring of the condition, or a combination thereof. If the subject is currently being treated for the condition with a course of treatment, the therapeutic intervention may comprise a subsequent different course of treatment (e.g., to increase treatment efficacy due to non-efficacy of the current course of treatment).

The therapeutic intervention may include prescribed medications or drugs, which may include one or more of: antimalarials, corticosteroids, immunosuppressants, and nonsteroidal anti-inflammatory drugs (NSAIDs). The therapeutic intervention may be effective to alleviate or decrease one or more symptoms, which may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof.

The therapeutic intervention may comprise recommending the subject for a secondary clinical test to confirm a diagnosis of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

The feature sets (e.g., comprising quantitative measures of a panel of condition-associated genomic loci) may be analyzed and assessed (e.g., using a trained algorithm comprising one or more classifiers) over a duration of time to monitor a patient (e.g., subject who has a condition or who is being treated for a condition). In such cases, the feature sets of the patient may change during the course of treatment. For example, the quantitative measures of the feature sets of a patient with decreasing risk of the condition due to an effective treatment may shift toward the profile or distribution of a healthy subject (e.g., a subject without the condition). Conversely, for example, the quantitative measures of the feature sets of a patient with increasing risk of the condition due to an ineffective treatment may shift toward the profile or distribution of a subject with higher risk of the condition or a more advanced stage or severity of the condition.

The condition of the subject may be monitored by monitoring a course of treatment for treating the condition of the subject. The monitoring may comprise assessing the condition of the subject at two or more time points. The assessing may be based at least on the feature sets (e.g., quantitative measures of a panel of condition-associated genomic loci) determined at each of the two or more time points. The therapeutic intervention may include prescribed medications or drugs, which may include one or more of: antimalarials, corticosteroids, immunosuppressants, and nonsteroidal anti-inflammatory drugs (NSAIDs). The therapeutic intervention may be effective to alleviate or decrease one or more symptoms, which may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof. The assessing may be based at least on the presence, absence, or severity of one or more symptoms, such as alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof.

In some embodiments, a difference in the feature sets (e.g., quantitative measures of a panel of condition-associated genomic loci) determined between the two or more time points may be indicative of one or more clinical indications, such as (i) a diagnosis of the condition of the subject, (ii) a prognosis of the condition of the subject, (iii) an increased risk of the condition of the subject, (iv) a decreased risk of the condition of the subject, (v) an efficacy of the course of treatment for treating the condition of the subject, and (vi) a non-efficacy of the course of treatment for treating the condition of the subject.

In some embodiments, a difference in the feature sets (e.g., quantitative measures of a panel of condition-associated genomic loci) determined between the two or more time points may be indicative of a diagnosis of the condition of the subject. For example, if the condition was not detected in the subject at an earlier time point but was detected in the subject at a later time point, then the difference is indicative of a diagnosis of the condition of the subject. A clinical action or decision may be made based on this indication of diagnosis of the condition of the subject, such as, for example, prescribing a new therapeutic intervention for the subject. The clinical action or decision may comprise recommending the subject for a secondary clinical test to confirm the diagnosis of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of a prognosis of the condition of the subject.

In some embodiments, a difference in the feature sets (e.g., quantitative measures of a panel of condition-associated genomic loci) determined between the two or more time points may be indicative of the subject having an increased risk of the condition. For example, if the condition was detected in the subject both at an earlier time point and at a later time point, and if the difference is a negative difference (e.g., the quantitative measures of a panel of condition-associated genomic loci increased from the earlier time point to the later time point), then the difference may be indicative of the subject having an increased risk of the condition. A clinical action or decision may be made based on this indication of the increased risk of the condition, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of the subject having a decreased risk of the condition. For example, if the condition was detected in the subject both at an earlier time point and at a later time point, and if the difference is a positive difference (e.g., the quantitative measures of a panel of condition-associated genomic loci decreased from the earlier time point to the later time point), then the difference may be indicative of the subject having a decreased risk of the condition. A clinical action or decision may be made based on this indication of the decreased risk of the condition (e.g., continuing or ending a current therapeutic intervention) for the subject. The clinical action or decision may comprise recommending the subject for a secondary clinical test to confirm the decreased risk of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of an efficacy of the course of treatment for treating the condition of the subject. For example, if the condition was detected in the subject at an earlier time point but was not detected in the subject at a later time point, then the difference may be indicative of an efficacy of the course of treatment for treating the condition of the subject. A clinical action or decision may be made based on this indication of the efficacy of the course of treatment for treating the condition of the subject, e.g., continuing or ending a current therapeutic intervention for the subject. The clinical action or decision may comprise recommending the subject for a secondary clinical test to confirm the efficacy of the course of treatment for treating the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of a non-efficacy of the course of treatment for treating the condition of the subject. For example, if the condition was detected in the subject both at an earlier time point and at a later time point, and if the difference is a negative or zero difference (e.g., the quantitative measures of a panel of condition-associated genomic loci increased or remained at a constant level from the earlier time point to the later time point), and if an efficacious treatment was indicated at an earlier time point, then the difference may be indicative of a non-efficacy of the course of treatment for treating the condition of the subject. A clinical action or decision may be made based on this indication of the non-efficacy of the course of treatment for treating the condition of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In various embodiments, machine learning methods are applied to distinguish samples in a population of samples. In one embodiment, machine learning methods are applied to distinguish samples between healthy and diseased (e.g., a lupus condition such as SLE or DLE) samples.

Kits

The present disclosure provides kits for identifying or monitoring a disease or disorder (e.g., a lupus condition) of a subject. A kit may comprise probes for identifying a quantitative measure (e.g., indicative of a presence, absence, or relative amount) of sequences at each of a panel of condition-associated genomic loci in a sample of the subject. A quantitative measure (e.g., indicative of a presence, absence, or relative amount) of sequences at each of a panel of condition-associated genomic loci in the sample may be indicative of the disease or disorder (e.g., a lupus condition) of the subject. The probes may be selective for the sequences at the panel of condition-associated genomic loci in the sample. A kit may comprise instructions for using the probes to process the sample to generate datasets indicative of a quantitative measure (e.g., indicative of a presence, absence, or relative amount) of sequences at each of the panel of condition-associated genomic loci in a sample of the subject.

The probes in the kit may be selective for the sequences at the panel of condition-associated genomic loci in the sample. The probes in the kit may be configured to selectively enrich nucleic acid (e.g., RNA or DNA) molecules corresponding to the panel of condition-associated genomic loci. The probes in the kit may be nucleic acid primers. The probes in the kit may have sequence complementarity with nucleic acid sequences from one or more of the panel of condition-associated genomic loci. The panel of condition-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more distinct condition-associated genomic loci.

The instructions in the kit may comprise instructions to assay the sample using the probes that are selective for the sequences at the panel of condition-associated genomic loci in the cell-free biological sample. These probes may be nucleic acid molecules (e.g., RNA or DNA) having sequence complementarity with nucleic acid sequences (e.g., RNA or DNA) from one or more of the plurality of panel of condition-associated genomic loci. These nucleic acid molecules may be primers or enrichment sequences. The instructions to assay the cell-free biological sample may comprise introductions to perform array hybridization, polymerase chain reaction (PCR), or nucleic acid sequencing (e.g., DNA sequencing or RNA sequencing) to process the sample to generate datasets indicative of a quantitative measure (e.g., indicative of a presence, absence, or relative amount) of sequences at each of the panel of condition-associated genomic loci in the sample. A quantitative measure (e.g., indicative of a presence, absence, or relative amount) of sequences at each of a panel of condition-associated genomic loci in the sample may be indicative of a disease or disorder (e.g., a lupus condition).

The instructions in the kit may comprise instructions to measure and interpret assay readouts, which may be quantified at one or more of the panel of condition-associated genomic loci to generate the datasets indicative of a quantitative measure (e.g., indicative of a presence, absence, or relative amount) of sequences at each of the panel of condition-associated genomic loci in the sample. For example, quantification of array hybridization or polymerase chain reaction (PCR) corresponding to the panel of condition-associated genomic loci may generate the datasets indicative of a quantitative measure (e.g., indicative of a presence, absence, or relative amount) of sequences at each of the panel of condition-associated genomic loci in the sample. Assay readouts may comprise quantitative PCR (qPCR) values, digital PCR (dPCR) values, digital droplet PCR (ddPCR) values, fluorescence values, etc., or normalized values thereof.

Analysis of Single Nucleotide Polymorphisms (SNPs) Associated with Lupus

Systemic lupus erythematosus (SLE) is a heterogeneous autoimmune disease that disproportionately affects subjects (e.g., women) of African-Ancestry (AA) compared to their European-Ancestral (EA) counterparts. This disparity may be further complicated by the fact that FDA-approved treatments for SLE, such as belimumab, may not provide a significant therapeutic benefit in SLE-affected AA subjects (e.g., women).

The present disclosure provides systems and methods to assess an SLE condition of a subject via analysis of data sets based on one or more ancestral groups of the subject. In various aspects, such systems and methods may be used to perform analysis of data sets including, for example, RNA gene expression or transcriptome data, or DNA genomic data.

In an aspect, the present disclosure provides a computer-implemented method for assessing an SLE condition of a subject, comprising: (a) receiving a dataset of a biological sample of the subject, wherein the dataset comprises quantitative measures of gene expression at each a plurality of SLE-associated genomic loci, wherein the plurality of SLE-associated genomic loci comprises (i) one or more AA-specific single nucleotide polymorphisms (SNPs) if the subject has an African-Ancestry (AA), or (ii) one or more EA-specific SNPs if the subject has a European-Ancestry (EA); (b) processing the dataset to identify one or more differentially expressed (DE) genomic loci among the plurality of SLE-associated genomic loci; and (c) based at least in part on the one or more DE genomic loci identified in (b) and whether the subject has an African-Ancestry (AA) or a European-Ancestry (EA), assessing the SLE condition of the subject.

In another aspect, the present disclosure provides a computer-implemented method for assessing an SLE condition of a subject, comprising: (a) receiving a dataset of a biological sample of the subject, wherein the dataset comprises quantitative measures of gene expression at each a plurality of systemic lupus erythematosus (SLE)-associated genomic loci, wherein the plurality of SLE-associated genomic loci comprises one or more African-Ancestry (AA)-specific single nucleotide polymorphisms (SNPs); (b) processing the dataset to identify one or more differentially expressed (DE) genomic loci among the plurality of SLE-associated genomic loci; and (c) based at least in part on the one or more DE genomic loci identified in (b) and whether the subject has an African-Ancestry (AA), assessing the SLE condition of the subject.

In another aspect, the present disclosure provides a computer-implemented method for assessing an SLE condition of a subject, comprising: (a) receiving a dataset of a biological sample of the subject, wherein the dataset comprises quantitative measures of gene expression at each a plurality of systemic lupus erythematosus (SLE)-associated genomic loci, wherein the plurality of SLE-associated genomic loci comprises one or more European-Ancestry (EA)-specific single nucleotide polymorphisms (SNPs); (b) processing the dataset to identify one or more differentially expressed (DE) genomic loci among the plurality of SLE-associated genomic loci; and (c) based at least in part on the one or more DE genomic loci identified in (b) and whether the subject has a European-Ancestry (EA), assessing the SLE condition of the subject.

In some embodiments, the dataset comprises RNA gene expression or transcriptome data, DNA genomic data, or a combination thereof. In some embodiments, the biological sample is selected from the group consisting of: a whole blood (WB) sample, a PBMC sample, a tissue sample, and a cell sample. In some embodiments, assessing the SLE condition of the subject comprises determining a diagnosis of the SLE condition, a prognosis of the SLE condition, a susceptibility of the SLE condition, a treatment for the SLE condition, or an efficacy or non-efficacy of a treatment for the SLE condition.

In some embodiments, the method further comprises determining a diagnosis of the SLE condition with a sensitivity of at least about 70%. In some embodiments, the method further comprises determining a diagnosis of the SLE condition with a specificity of at least about 70%. In some embodiments, the method further comprises determining a diagnosis of the SLE condition with a positive predictive value of at least about 70%. In some embodiments, the method further comprises determining a diagnosis of the SLE condition with a negative predictive value of at least about 70%. In some embodiments, the method further comprises determining a diagnosis of the SLE condition with an Area Under Curve (AUC) of at least about 70%. In some embodiments, the method further comprises determining a likelihood of the diagnosis of the SLE condition of the subject.

In some embodiments, the method further comprises generating a plurality of drug candidates for the SLE condition of the subject. In some embodiments, the method further comprises evaluating or predicting a relative efficacy of the plurality of drug candidates for the SLE condition of the subject. In some embodiments, the method further comprises providing a therapeutic intervention comprising one or more of the plurality of drug candidates for the SLE condition of the subject.

In some embodiments, the method further comprises selecting a treatment for the SLE condition of the subject, the treatment comprising an AA-specific drug. In some embodiments, the AA-specific drug is selected from the group consisting of: an HDAC inhibitor, a retinoid, a IRAK4-targeted drug, and a CTLA4-targeted drug. In some embodiments, the method further comprises selecting a treatment for the SLE condition of the subject, the treatment comprising an EA-specific drug. In some embodiments, the EA-specific drug is selected from the group consisting of: hydroxychloroquine, a CD40LG-targeted drug, a CXCR1-targeted drug, and a CXCR2-targeted drug. In some embodiments, the method further comprises selecting a treatment for the SLE condition of the subject, the treatment comprising a drug targeting E-Genes or pathways shared by EA and AA. In some embodiments, the drug targeting E-Genes or pathways shared by EA and AA is selected from the group consisting of: ibrutinib, ruxolitinib, and ustekinumab.

In some embodiments, the method further comprises monitoring the SLE condition of the subject, wherein the monitoring comprises assessing the SLE condition of the subject at each of a plurality of time points, and processing the plurality of assessments of the SLE condition of the subject at each of the plurality of time points.

In some embodiments, the one or more EA-specific SNPs comprise one or more SNPs of genes selected from the group listed in Table 56. In some embodiments, the one or more AA-specific SNPs comprise one or more SNPs of genes selected from the group listed in Table 57. In some embodiments, the plurality of SLE-associated genomic loci comprises one or more shared SNPs, wherein the one or more shared SNPs are common to both EA and AA. In some embodiments, the one or more shared SNPs comprise one or more SNPs of genes selected from the group listed in Table 58.

In another aspect, the present disclosure provides a computer system for assessing an SLE condition of a subject, comprising: a database that is configured to store an African-Ancestry (AA) status of the subject, a European-Ancestry (EA) status of the subject, and a dataset of a biological sample of the subject, wherein the dataset comprises quantitative measures of gene expression at each a plurality of SLE-associated genomic loci, wherein the plurality of SLE-associated genomic loci comprises (i) one or more AA-specific single nucleotide polymorphisms (SNPs) if the subject has an African-Ancestry (AA), or (ii) one or more EA-specific SNPs if the subject has a European-Ancestry (EA); and one or more computer processors operatively coupled to the database, wherein the one or more computer processors are individually or collectively programmed to: (i) process the dataset to identify one or more differentially expressed (DE) genomic loci among the plurality of SLE-associated genomic loci; and (ii) based at least in part on the one or more DE genomic loci identified in (ii), the AA status of the subject, and the EA status of the subject, assessing the SLE condition of the subject.

In another aspect, the present disclosure provides a computer system for assessing an SLE condition of a subject, comprising: a database that is configured to store an African-Ancestry (AA) status of the subject and a dataset of a biological sample of the subject, wherein the dataset comprises quantitative measures of gene expression at each a plurality of systemic lupus erythematosus (SLE)-associated genomic loci, wherein the plurality of SLE-associated genomic loci comprises one or more African-Ancestry (AA)-specific single nucleotide polymorphisms (SNPs); and one or more computer processors operatively coupled to the database, wherein the one or more computer processors are individually or collectively programmed to: (i) process the dataset to identify one or more differentially expressed (DE) genomic loci among the plurality of SLE-associated genomic loci; and (ii) based at least in part on the one or more DE genomic loci identified in (ii) and the AA status of the subject, assessing the SLE condition of the subject.

In another aspect, the present disclosure provides a computer system for assessing an SLE condition of a subject, comprising: a database that is configured to store a European-Ancestry (EA) status of the subject and a dataset of a biological sample of the subject, wherein the dataset comprises quantitative measures of gene expression at each a plurality of systemic lupus erythematosus (SLE)-associated genomic loci, wherein the plurality of SLE-associated genomic loci comprises one or more European-Ancestry (EA)-specific single nucleotide polymorphisms (SNPs); and one or more computer processors operatively coupled to the database, wherein the one or more computer processors are individually or collectively programmed to: (i) process the dataset to identify one or more differentially expressed (DE) genomic loci among the plurality of SLE-associated genomic loci; and (ii) based at least in part on the one or more DE genomic loci identified in (i) and the EA status of the subject, assess the SLE condition of the subject.

In another aspect, the present disclosure provides a non-transitory computer readable medium comprising machine-executable code that, upon execution by one or more computer processors, implements a method for assessing an SLE condition of a subject, comprising: (a) receiving a dataset of a biological sample of the subject, wherein the dataset comprises quantitative measures of gene expression at each a plurality of SLE-associated genomic loci, wherein the plurality of SLE-associated genomic loci comprises (i) one or more AA-specific single nucleotide polymorphisms (SNPs) if the subject has an African-Ancestry (AA), or (ii) one or more EA-specific SNPs if the subject has a European-Ancestry (EA); (b) processing the dataset to identify one or more differentially expressed (DE) genomic loci among the plurality of SLE-associated genomic loci; and (c) based at least in part on the one or more DE genomic loci identified in (b) and whether the subject has an African-Ancestry (AA) or a European-Ancestry (EA), assessing the SLE condition of the subject.

In another aspect, the present disclosure provides a non-transitory computer readable medium comprising machine-executable code that, upon execution by one or more computer processors, implements a method for assessing an SLE condition of a subject, comprising: (a) receiving a dataset of a biological sample of the subject, wherein the dataset comprises quantitative measures of gene expression at each a plurality of systemic lupus erythematosus (SLE)-associated genomic loci, wherein the plurality of SLE-associated genomic loci comprises one or more African-Ancestry (AA)-specific single nucleotide polymorphisms (SNPs); (b) processing the dataset to identify one or more differentially expressed (DE) genomic loci among the plurality of SLE-associated genomic loci; and (c) based at least in part on the one or more DE genomic loci identified in (b) and whether the subject has an African-Ancestry (AA), assessing the SLE condition of the subject.

In another aspect, the present disclosure provides a non-transitory computer readable medium comprising machine-executable code that, upon execution by one or more computer processors, implements a method for assessing an SLE condition of a subject, comprising: (a) receiving a dataset of a biological sample of the subject, wherein the dataset comprises quantitative measures of gene expression at each a plurality of systemic lupus erythematosus (SLE)-associated genomic loci, wherein the plurality of SLE-associated genomic loci comprises one or more European-Ancestry (EA)-specific single nucleotide polymorphisms (SNPs); (b) processing the dataset to identify one or more differentially expressed (DE) genomic loci among the plurality of SLE-associated genomic loci; and (c) based at least in part on the one or more DE genomic loci identified in (b) and whether the subject has a European-Ancestry (EA) assessing the SLE condition of the subject.

Assessment of SLE Conditions

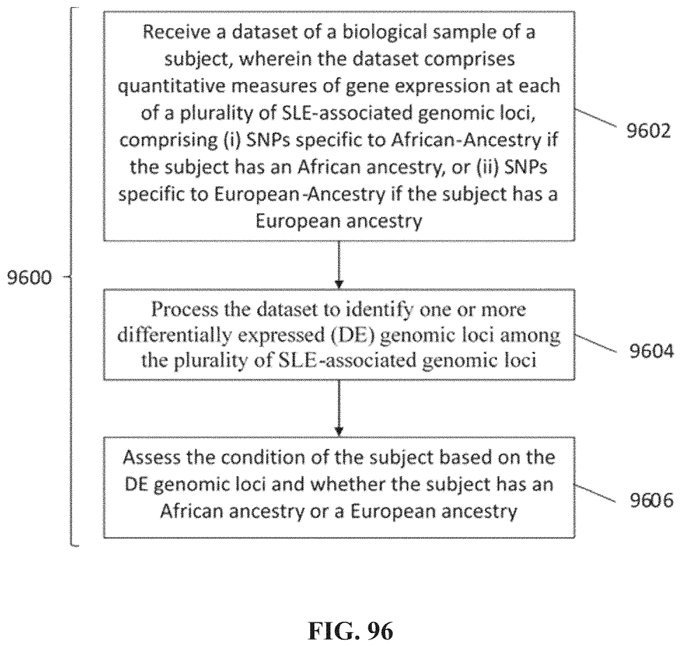

FIG. 96 shows a non-limiting example of a method 9600 to assess an SLE condition of a subject, in accordance with disclosed embodiments. In operation 9602, a dataset of a biological sample of a subject is received. The dataset may comprise quantitative measures of gene expression at each of a plurality of SLE-associated genomic loci. The plurality of SLE-associated genomic loci may comprise (i) SNPs specific to African-Ancestry (AA) if the subject has an African ancestry, or (ii) SNPs specific to European-Ancestry (EA) if the subject has a European ancestry. In operation 9604, the dataset is processed to identify one or more differentially expressed (DE) genomic loci among the plurality of SLE-associated genomic loci. In operation 9606, the SLE condition of the subject is assessed based on the DE genomic loci and whether the subject has an African ancestry or a European ancestry.

To obtain a blood sample, various techniques may be used, e.g., a syringe or other vacuum suction device. A blood sample can be optionally pre-treated or processed prior to use. A sample, such as a blood sample, may be analyzed under any of the methods and systems herein within 4 weeks, 2 weeks, 1 week, 6 days, 5 days, 4 days, 3 days, 2 days, 1 day, 12 hr, 6 hr, 3 hr, 2 hr, or 1 hr from the time the sample is obtained, or longer if frozen. When obtaining a sample from a subject (e.g., blood sample), the amount can vary depending upon subject size and the condition being screened. In some embodiments, at least 10 mL, 5 mL, 1 mL, 0.5 mL, 250, 200, 150, 100, 50, 40, 30, 20, 10, 9, 8, 7, 6, 5, 4, 3, 2, or 1 μL of a sample is obtained. In some embodiments, 1-50, 2-40, 3-30, or 4-20 μL of sample is obtained. In some embodiments, more than 5, 10, 15, 20, 25, 30, 35, 40, 45, 50, 55, 60, 65, 70, 75, 80, 85, 90, 95 or 100 μL of a sample is obtained.

The sample may be taken before and/or after treatment of a subject with a disease or disorder. Samples may be obtained from a subject during a treatment or a treatment regime. Multiple samples may be obtained from a subject to monitor the effects of the treatment over time. The sample may be taken from a subject known or suspected of having a disease or disorder for which a definitive positive or negative diagnosis is not available via clinical tests. The sample may be taken from a subject suspected of having a disease or disorder. The sample may be taken from a subject experiencing unexplained symptoms, such as fatigue, nausea, weight loss, aches and pains, weakness, or bleeding. The sample may be taken from a subject having explained symptoms. The sample may be taken from a subject at risk of developing a disease or disorder due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

In some embodiments, a sample can be taken at a first time point and assayed, and then another sample can be taken at a subsequent time point and assayed. Such methods can be used, for example, for longitudinal monitoring purposes to track the development or progression of a disease or disorder (e.g., an SLE condition). In some embodiments, the progression of a disease can be tracked before treatment, after treatment, or during the course of treatment, to determine the treatment's effectiveness. For example, a method as described herein can be performed on a subject prior to, and after, treatment with an SLE therapy to measure the disease's progression or regression in response to the SLE therapy.

After obtaining a sample from the subject, the sample may be processed to generate datasets indicative of a condition (e.g., an SLE condition) of the subject. For example, a presence, absence, or quantitative assessment of nucleic acid molecules of the sample at a panel of condition-associated (e.g., SLE-associated) genomic loci or may be indicative of a condition (e.g., an SLE condition) of the subject. Processing the sample obtained from the subject may comprise (i) subjecting the sample to conditions that are sufficient to isolate, enrich, or extract a plurality of nucleic acid molecules, and (ii) assaying the plurality of nucleic acid molecules to generate the dataset (e.g., microarray data, nucleic acid sequences, or quantitative polymerase chain reaction (qPCR) data). Methods of assaying may include any assay known in the art or described in the literature, for example, a microarray assay, a sequencing assay (e.g., DNA sequencing, RNA sequencing, or RNA-Seq), or a quantitative polymerase chain reaction (qPCR) assay.

In some embodiments, a plurality of nucleic acid molecules is extracted from the sample and subjected to sequencing to generate a plurality of sequencing reads. The nucleic acid molecules may comprise ribonucleic acid (RNA) or deoxyribonucleic acid (DNA). The extraction method may extract all RNA or DNA molecules from a sample. Alternatively, the extraction method may selectively extract a portion of RNA or DNA molecules from a sample. Extracted RNA molecules from a sample may be converted to cDNA molecules by reverse transcription (RT).

The sample may be processed without any nucleic acid extraction. For example, the disease or disorder may be identified or monitored in the subject by using probes configured to selectively enrich nucleic acid (e.g., RNA or DNA) molecules corresponding to a panel of SLE-associated genomic loci. The probes may be nucleic acid primers. The probes may have sequence complementarity with nucleic acid sequences from one or more of the panel of condition-associated (e.g., SLE-associated) genomic loci. The panel of condition-associated genomic loci may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more condition-associated genomic loci.

The probes may be nucleic acid molecules (e.g., RNA or DNA) having sequence complementarity with nucleic acid sequences (e.g., RNA or DNA) of one or more genomic loci (e.g., condition-associated genomic loci). These nucleic acid molecules may be primers or enrichment sequences. The assaying of the sample using probes that are selective for the one or more genomic loci (e.g., condition-associated genomic loci) may comprise use of array hybridization, polymerase chain reaction (PCR), or nucleic acid sequencing (e.g., RNA sequencing or DNA sequencing, such as RNA-Seq).

The assay readouts may be quantified at one or more genomic loci (e.g., condition-associated genomic loci) to generate the data indicative of the disease or disorder. For example, quantification of array hybridization or polymerase chain reaction (PCR) corresponding to a plurality of genomic loci (e.g., condition-associated genomic loci) may generate data indicative of the disease or disorder. Assay readouts may comprise quantitative PCR (qPCR) values, digital PCR (dPCR) values, digital droplet PCR (ddPCR) values, fluorescence values, etc., or normalized values thereof.

Classifiers

In some embodiments, the present disclosure provides a system, method, or kit having data analysis realized in software application, computing hardware, or both. In various embodiments, the analysis application or system includes at least a data receiving module, a data pre-processing module, a data analysis module, a data interpretation module, or a data visualization module. In one embodiment, the data receiving module can comprise computer systems that connect laboratory hardware or instrumentation with computer systems that process laboratory data. In one embodiment, the data pre-processing module can comprise hardware systems or computer software that performs operations on the data in preparation for analysis. Examples of operations that can be applied to the data in the pre-processing module include affine transformations, denoising operations, data cleaning, reformatting, or subsampling. A data analysis module, which can be specialized for analyzing genomic data from one or more genomic materials, can, for example, take assembled genomic sequences and perform probabilistic and statistical analysis to identify abnormal patterns related to a disease, pathology, state, risk, condition, or phenotype. A data interpretation module can use analysis methods, for example, drawn from statistics, mathematics, or biology, to support understanding of the relation between the identified abnormal patterns and health conditions, functional states, prognoses, or risks. A data visualization module can use methods of mathematical modeling, computer graphics, or rendering to create visual representations of data that can facilitate the understanding or interpretation of results.

Feature sets may be generated from datasets obtained using one or more assays of a biological sample obtained or derived from a subject, and a trained algorithm may be used to process one or more of the feature sets to identify or assess a condition (e.g., a disease or disorder, such as an SLE condition) of a subject. For example, the trained algorithm may be used to apply a machine learning classifier to a plurality of condition-associated genomic loci that are associated with two or more classes of individuals inputted into a machine learning model, in order to classify a subject into one of the two or more classes of individuals. For example, the trained algorithm may be used to apply a machine learning classifier to a plurality of condition-associated (e.g., SLE-associated) that are associated with individuals with known conditions (e.g., a disease or disorder, such as an SLE condition) and individuals not having the condition (e.g., healthy individuals, or individuals who do not have an SLE condition), in order to classify a subject as having the condition (e.g., positive test outcome) or not having the condition (e.g., negative test outcome).

The trained algorithm may be configured to identify the presence (e.g., positive test result) or absence (e.g., negative test result) of one or more conditions (e.g., a disease or disorder, such as an SLE condition) with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99%. This accuracy may be achieved for a set of at least about 25, at least about 50, at least about 100, at least about 150, at least about 200, at least about 250, at least about 300, at least about 350, at least about 400, at least about 450, at least about 500, at least about 1,000, or more than about 1,000 independent samples.

The trained algorithm may comprise a machine learning algorithm, such as a supervised machine learning algorithm. The supervised machine learning algorithm may comprise, for example, a Random Forest, a support vector machine (SVM), a neural network, or a deep learning algorithm. The trained algorithm may comprise a classification and regression tree (CART) algorithm. The trained algorithm may comprise an unsupervised machine learning algorithm.

The trained algorithm may comprise a classifier configured to accept as input a plurality of input variables or features (e.g., condition-associated (e.g., SLE-associated) genomic loci) and to produce or output one or more output values based on the plurality of input variables or features (e.g., condition-associated genomic loci). The plurality of input variables or features may comprise one or more datasets indicative of the presence (e.g., positive test result) or absence (e.g., negative test result) of one or more conditions (e.g., a disease or disorder, such as an SLE condition). For example, an input variable or feature may comprise a number of sequences corresponding to or aligning to each of the plurality of condition-associated genomic loci.

The plurality of input variables or features may also include clinical information of a subject, such as health data. For example, the health data of a subject may comprise one or more of: a diagnosis of one or more conditions (e.g., a disease or disorder, such as an SLE condition), a prognosis of one or more conditions (e.g., a disease or disorder, such as an SLE condition), a risk of having one or more conditions (e.g., a disease or disorder, such as an SLE condition), a treatment history of one or more conditions (e.g., a disease or disorder, such as an SLE condition), a history of previous treatment for one or more conditions (e.g., a disease or disorder, such as an SLE condition), a history of prescribed medications, a history of prescribed medical devices, smoking status, age, height, weight, sex, race, ethnicity, nationality, African-Ancestry (AA) status, European-Ancestry (EA) status, and one or more symptoms of the subject.

For example, the disease or disorder may comprise one or more of: systemic lupus erythematosus (SLE), discoid lupus erythematosus (DLE), and lupus nephritis (LN). As another example, the symptoms may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof. As another example, the prescribed medications or drugs may include one or more of: antimalarials, corticosteroids, immunosuppressants, and nonsteroidal anti-inflammatory drugs (NSAIDs).

The trained algorithm may comprise a classifier, such that each of the one or more output values comprises one of a fixed number of possible values (e.g., a linear classifier, a logistic regression classifier, etc.) indicating a classification of the sample by the classifier. The trained algorithm may comprise a binary classifier, such that each of the one or more output values comprises one of two values (e.g., {0, 1}, {positive, negative}, or {high-risk, low-risk}) indicating a classification of the sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the sample by the classifier.

The classifier may be configured to classify samples by assigning output values, which may comprise descriptive labels, numerical values, or a combination thereof. Some of the output values may comprise descriptive labels. Such descriptive labels may provide an identification or indication of the presence (e.g., positive test result) or absence (e.g., negative test result) of one or more conditions (e.g., a disease or disorder, such as an SLE condition) of the subject, and may comprise, for example, positive, negative, high-risk, intermediate-risk, low-risk, or indeterminate. Such descriptive labels may provide an identification of a treatment for the one or more conditions of the subject, and may comprise, for example, a therapeutic intervention, a duration of the therapeutic intervention, and/or a dosage of the therapeutic intervention suitable to treat the one or more conditions of the subject. Such descriptive labels may provide an identification of secondary clinical tests that may be appropriate to perform on the subject, and may comprise, for example, an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof. For example, such descriptive labels may provide a prognosis of the one or more conditions of the subject. As another example, such descriptive labels may provide a relative assessment of the one or more conditions of the subject. Some descriptive labels may be mapped to numerical values, for example, by mapping “positive” to 1 and “negative” to 0.

The classifier may be configured to classify samples by assigning output values that comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the one or more conditions (e.g., a disease or disorder, such as an SLE condition) of the subject. Some numerical values may be mapped to descriptive labels, for example, by mapping 1 to “positive” and 0 to “negative.”

The classifier may be configured to classify samples by assigning output values based on one or more cutoff values. For example, a binary classification of samples may assign an output value of “positive” or 1 if the sample indicates that the subject has at least a 50% probability of having one or more conditions (e.g., a disease or disorder, such as an SLE condition), thereby assigning the subject to a class of individuals receiving a positive test result. As another example, a binary classification of samples may assign an output value of “negative” or 0 if the sample indicates that the subject has less than a 50% probability of having one or more conditions (e.g., a disease or disorder), thereby assigning the subject to a class of individuals receiving a negative test result. In this case, a single cutoff value of 50% is used to classify samples into one of the two possible binary output values or classes of individuals (e.g., those receiving a positive test result and those receiving a negative test result). Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, the classifier may be configured to classify samples by assigning an output value of “positive” or 1 if the sample indicates that the subject has a probability of having one or more conditions (e.g., a disease or disorder, such as an SLE condition) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of having one or more conditions (e.g., a disease or disorder, such as an SLE condition) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classifier may be configured to classify samples by assigning an output value of “negative” or 0 if the sample indicates that the subject has a probability of having one or more conditions (e.g., a disease or disorder, such as an SLE condition) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having one or more conditions (e.g., a disease or disorder, such as an SLE condition) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classifier may be configured to classify samples by assigning an output value of “indeterminate” or 2 if the sample is not classified as “positive”, “negative”, 1, or 0. In this case, a set of two cutoff values is used to classify samples into one of the three possible output values or classes of individuals (e.g., corresponding to outcome groups of individuals having “low risk,” “intermediate risk,” and “high risk” of having one or more conditions, such as a disease or disorder). Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values or classes of individuals, where n is any positive integer.

The trained algorithm may be trained with a plurality of independent training samples. Each of the independent training samples may comprise a sample from a subject, associated datasets obtained by assaying the sample (as described elsewhere herein), and one or more known output values or classes of individuals corresponding to the sample (e.g., a clinical diagnosis, prognosis, absence, or treatment efficacy of a condition of the subject). Independent training samples may comprise samples and associated datasets and outputs obtained or derived from a plurality of different subjects. Independent training samples may comprise samples and associated datasets and outputs obtained at a plurality of different time points from the same subject (e.g., on a regular basis such as weekly, biweekly, or monthly), as part of a longitudinal monitoring of a subject before, during, and after a course of treatment for one or more conditions of the subject. Independent training samples may be associated with presence of the condition (e.g., training samples comprising samples and associated datasets and outputs obtained or derived from a plurality of subjects known to have the condition). Independent training samples may be associated with absence of the condition (e.g., training samples comprising samples and associated datasets and outputs obtained or derived from a plurality of subjects who are known to not have a previous diagnosis of the condition or who have received a negative test result for the condition).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise samples associated with presence of the condition and/or samples associated with absence of the condition. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the condition (e.g., a disease or disorder, such as an SLE condition). The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with absence of the condition (e.g., a disease or disorder, such as an SLE condition). In some embodiments, the sample is independent of samples used to train the trained algorithm.

The trained algorithm may be trained with a first number of independent training samples associated with a presence of the condition (e.g., a disease or disorder, such as an SLE condition) and a second number of independent training samples associated with an absence of the condition (e.g., a disease or disorder, such as an SLE condition). The first number of independent training samples associated with presence of the condition (e.g., a disease or disorder, such as an SLE condition) may be no more than the second number of independent training samples associated with absence of the condition (e.g., a disease or disorder, such as an SLE condition). The first number of independent training samples associated with a presence of the condition (e.g., a disease or disorder) may be equal to the second number of independent training samples associated with an absence of the condition (e.g., a disease or disorder, such as an SLE condition). The first number of independent training samples associated with a presence of the condition (e.g., a disease or disorder, such as an SLE condition) may be greater than the second number of independent training samples associated with an absence of the condition (e.g., a disease or disorder, such as an SLE condition).

The trained algorithm may comprise a classifier configured to identify the presence (e.g., positive test result) or absence (e.g., negative test result) of one or more conditions (e.g., a disease or disorder, such as an SLE condition) with an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the presence (e.g., positive test result) or absence (e.g., negative test result) of the one or more conditions by the trained algorithm may be calculated as the percentage of independent test samples (e.g., subjects known to have the condition or subjects with negative clinical test results for the condition) that are correctly identified or classified as having or not having the condition.

The trained algorithm may comprise a classifier configured to identify one or more conditions (e.g., a disease or disorder, such as an SLE condition) with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the condition using the trained algorithm may be calculated as the percentage of samples identified or classified as having the condition that correspond to subjects that truly have the condition.

The trained algorithm may comprise a classifier configured to identify one or more conditions (e.g., a disease or disorder, such as an SLE condition) with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the condition using the trained algorithm may be calculated as the percentage of samples identified or classified as not having the condition that correspond to subjects that truly do not have the condition.

The trained algorithm may comprise a classifier configured to identify one or more conditions (e.g., a disease or disorder, such as an SLE condition) with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the condition using the trained algorithm may be calculated as the percentage of independent test samples associated with presence of the condition (e.g., subjects known to have the condition) that are correctly identified or classified as having the condition.

The trained algorithm may comprise a classifier configured to identify one or more conditions (e.g., a disease or disorder, such as an SLE condition) with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the condition using the trained algorithm may be calculated as the percentage of independent test samples associated with absence of the condition (e.g., subjects with negative clinical test results for the condition) that are correctly identified or classified as not having the condition.

The trained algorithm may comprise a classifier configured to identify the presence (e.g., positive test result) or absence (e.g., negative test result) of one or more conditions (e.g., a disease or disorder, such as an SLE condition) with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying samples as having or not having the condition.

Classifiers of the trained algorithm may be adjusted or tuned to improve or optimize one or more performance metrics, such as accuracy, PPV, NPV, clinical sensitivity, clinical specificity, AUC, or a combination thereof (e.g., a performance index incorporating a plurality of such performance metrics, such as by calculating a weight sum therefrom), of identifying the presence (e.g., positive test result) or absence (e.g., negative test result) of the condition. The classifiers may be adjusted or tuned by adjusting parameters of the classifiers (e.g., a set of cutoff values used to classify a sample as described elsewhere herein, or weights of a neural network) to improve or optimize the performance metrics. The one or more classifiers may be adjusted or tuned so as to reduce an overall classification error (e.g., an “out-of-bag” or oob error rate for a Random Forest classifier). The one or more classifiers may be adjusted or tuned continuously during the training process (e.g., as sample datasets are added to the training set) or after the training process has completed.

The trained algorithm may comprise a plurality of classifiers (e.g., an ensemble) such that the plurality of classifications or outcome values of the plurality of classifiers may be combined to produce a single classification or outcome value for the sample. For example, a sum or a weighted sum of the plurality of classifications or outcome values of the plurality of classifiers may be calculated to produce a single classification or outcome value for the sample. As another example, a majority vote of the plurality of classifications or outcome values of the plurality of classifiers may be identified to produce a single classification or outcome value for the sample. In this manner, a single classification or outcome value may be produced for the sample having greater confidence or statistical significance than the individual classifications or outcome values produced by each of the plurality of classifiers.

After the trained algorithm is initially trained, a subset of the inputs may be identified as most influential or most important to be included for making high-quality classifications (e.g., having highest permutation feature importance). For example, a subset of the panel of condition-associated genomic loci may be identified as most influential or most important to be included for making high-quality classifications or identifications of conditions (or sub-types of conditions). The panel of condition-associated genomic loci, or a subset thereof, may be ranked based on classification metrics indicative of each influence or importance of each individual condition-associated genomic locus toward making high-quality classifications or identifications of conditions (or sub-types of conditions). Such metrics may be used to reduce, in some cases significantly, the number of input variables (e.g., predictor variables) that may be used to train the one or more classifiers of the trained algorithm to a desired performance level (e.g., based on a desired minimum accuracy, PPV, NPV, clinical sensitivity, clinical specificity, AUC, or a combination thereof).

For example, if training a classifier of the trained algorithm with a plurality comprising several dozen or hundreds of input variables to the classifier results in an accuracy of classification of more than 99%, then training the classifier of the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

As another example, if training a classifier of the trained algorithm with a plurality comprising several dozen or hundreds of input variables to the classifier results in a sensitivity or specificity of classification of more than 99%, then training the classifier of the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable sensitivity or specificity of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

The subset of the plurality of input variables (e.g., the panel of condition-associated genomic loci) to the classifier of the trained algorithm may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics (e.g., permutation feature importance).

Upon identifying the subject as having one or more conditions (e.g., a disease or disorder, such as an SLE condition), the subject may be optionally provided with a therapeutic intervention (e.g., prescribing an appropriate course of treatment to treat the one or more conditions of the subject). The therapeutic intervention may comprise a prescription of an effective dose of a drug, a further testing or evaluation of the condition, a further monitoring of the condition, or a combination thereof. If the subject is currently being treated for the condition with a course of treatment, the therapeutic intervention may comprise a subsequent different course of treatment (e.g., to increase treatment efficacy due to non-efficacy of the current course of treatment).

The therapeutic intervention may include prescribed medications or drugs, which may include one or more of: antimalarials, corticosteroids, immunosuppressants, and nonsteroidal anti-inflammatory drugs (NSAIDs). The therapeutic intervention may be effective to alleviate or decrease one or more symptoms, which may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof.

The therapeutic intervention may comprise recommending the subject for a secondary clinical test to confirm a diagnosis of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

The feature sets (e.g., comprising quantitative measures of a panel of condition-associated genomic loci) may be analyzed and assessed (e.g., using a trained algorithm comprising one or more classifiers) over a duration of time to monitor a patient (e.g., subject who has a condition or who is being treated for a condition). In such cases, the feature sets of the patient may change during the course of treatment. For example, the quantitative measures of the feature sets of a patient with decreasing risk of the condition due to an effective treatment may shift toward the profile or distribution of a healthy subject (e.g., a subject without the condition). Conversely, for example, the quantitative measures of the feature sets of a patient with increasing risk of the condition due to an ineffective treatment may shift toward the profile or distribution of a subject with higher risk of the condition or a more advanced stage or severity of the condition.

The condition of the subject may be monitored by monitoring a course of treatment for treating the condition of the subject. The monitoring may comprise assessing the condition of the subject at two or more time points. The assessing may be based at least on the feature sets (e.g., quantitative measures of a panel of condition-associated genomic loci) determined at each of the two or more time points. The therapeutic intervention may include prescribed medications or drugs, which may include one or more of: antimalarials, corticosteroids, immunosuppressants, and nonsteroidal anti-inflammatory drugs (NSAIDs). The therapeutic intervention may be effective to alleviate or decrease one or more symptoms, which may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof. The assessing may be based at least on the presence, absence, or severity of one or more symptoms, such as alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof.

In some embodiments, a difference in the feature sets (e.g., quantitative measures of a panel of condition-associated genomic loci) determined between the two or more time points may be indicative of one or more clinical indications, such as (i) a diagnosis of the condition of the subject, (ii) a prognosis of the condition of the subject, (iii) an increased risk of the condition of the subject, (iv) a decreased risk of the condition of the subject, (v) an efficacy of the course of treatment for treating the condition of the subject, and (vi) a non-efficacy of the course of treatment for treating the condition of the subject.

In some embodiments, a difference in the feature sets (e.g., quantitative measures of a panel of condition-associated genomic loci) determined between the two or more time points may be indicative of a diagnosis of the condition of the subject. For example, if the condition was not detected in the subject at an earlier time point but was detected in the subject at a later time point, then the difference is indicative of a diagnosis of the condition of the subject. A clinical action or decision may be made based on this indication of diagnosis of the condition of the subject, such as, for example, prescribing a new therapeutic intervention for the subject. The clinical action or decision may comprise recommending the subject for a secondary clinical test to confirm the diagnosis of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of a prognosis of the condition of the subject.

In some embodiments, a difference in the feature sets (e.g., quantitative measures of a panel of condition-associated genomic loci) determined between the two or more time points may be indicative of the subject having an increased risk of the condition. For example, if the condition was detected in the subject both at an earlier time point and at a later time point, and if the difference is a negative difference (e.g., the quantitative measures of a panel of condition-associated genomic loci increased from the earlier time point to the later time point), then the difference may be indicative of the subject having an increased risk of the condition. A clinical action or decision may be made based on this indication of the increased risk of the condition, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of the subject having a decreased risk of the condition. For example, if the condition was detected in the subject both at an earlier time point and at a later time point, and if the difference is a positive difference (e.g., the quantitative measures of a panel of condition-associated genomic loci decreased from the earlier time point to the later time point), then the difference may be indicative of the subject having a decreased risk of the condition. A clinical action or decision may be made based on this indication of the decreased risk of the condition (e.g., continuing or ending a current therapeutic intervention) for the subject. The clinical action or decision may comprise recommending the subject for a secondary clinical test to confirm the decreased risk of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of an efficacy of the course of treatment for treating the condition of the subject. For example, if the condition was detected in the subject at an earlier time point but was not detected in the subject at a later time point, then the difference may be indicative of an efficacy of the course of treatment for treating the condition of the subject. A clinical action or decision may be made based on this indication of the efficacy of the course of treatment for treating the condition of the subject, e.g., continuing or ending a current therapeutic intervention for the subject. The clinical action or decision may comprise recommending the subject for a secondary clinical test to confirm the efficacy of the course of treatment for treating the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of a non-efficacy of the course of treatment for treating the condition of the subject. For example, if the condition was detected in the subject both at an earlier time point and at a later time point, and if the difference is a negative or zero difference (e.g., the quantitative measures of a panel of condition-associated genomic loci increased or remained at a constant level from the earlier time point to the later time point), and if an efficacious treatment was indicated at an earlier time point, then the difference may be indicative of a non-efficacy of the course of treatment for treating the condition of the subject. A clinical action or decision may be made based on this indication of the non-efficacy of the course of treatment for treating the condition of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In various embodiments, machine learning methods are applied to distinguish samples in a population of samples. In one embodiment, machine learning methods are applied to distinguish samples between healthy and diseased (e.g., an SLE condition such as SLE or DLE) samples.

Kits

The present disclosure provides kits for identifying or monitoring a disease or disorder (e.g., an SLE condition) of a subject. A kit may comprise probes for identifying a quantitative measure (e.g., indicative of a presence, absence, or relative amount) of sequences at each of a panel of condition-associated (e.g., SLE-associated) genomic loci in a sample of the subject. A quantitative measure (e.g., indicative of a presence, absence, or relative amount) of sequences at each of a panel of condition-associated genomic loci in the sample may be indicative of the disease or disorder (e.g., an SLE condition) of the subject. The probes may be selective for the sequences at the panel of condition-associated genomic loci in the sample. A kit may comprise instructions for using the probes to process the sample to generate datasets indicative of a quantitative measure (e.g., indicative of a presence, absence, or relative amount) of sequences at each of the panel of condition-associated genomic loci in a sample of the subject.

The probes in the kit may be selective for the sequences at the panel of condition-associated (e.g., SLE-associated) genomic loci in the sample. The probes in the kit may be configured to selectively enrich nucleic acid (e.g., RNA or DNA) molecules corresponding to the panel of condition-associated genomic loci. The probes in the kit may be nucleic acid primers. The probes in the kit may have sequence complementarity with nucleic acid sequences from one or more of the panel of condition-associated genomic loci. The panel of condition-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more distinct condition-associated genomic loci.

The instructions in the kit may comprise instructions to assay the sample using the probes that are selective for the sequences at the panel of condition-associated (e.g., SLE-associated) genomic loci in the cell-free biological sample. These probes may be nucleic acid molecules (e.g., RNA or DNA) having sequence complementarity with nucleic acid sequences (e.g., RNA or DNA) from one or more of the plurality of panel of condition-associated genomic loci. These nucleic acid molecules may be primers or enrichment sequences. The instructions to assay the cell-free biological sample may comprise introductions to perform array hybridization, polymerase chain reaction (PCR), or nucleic acid sequencing (e.g., DNA sequencing or RNA sequencing) to process the sample to generate datasets indicative of a quantitative measure (e.g., indicative of a presence, absence, or relative amount) of sequences at each of the panel of condition-associated genomic loci in the sample. A quantitative measure (e.g., indicative of a presence, absence, or relative amount) of sequences at each of a panel of condition-associated genomic loci in the sample may be indicative of a disease or disorder (e.g., an SLE condition).

The instructions in the kit may comprise instructions to measure and interpret assay readouts, which may be quantified at one or more of the panel of condition-associated genomic loci to generate the datasets indicative of a quantitative measure (e.g., indicative of a presence, absence, or relative amount) of sequences at each of the panel of condition-associated genomic loci in the sample. For example, quantification of array hybridization or polymerase chain reaction (PCR) corresponding to the panel of condition-associated genomic loci may generate the datasets indicative of a quantitative measure (e.g., indicative of a presence, absence, or relative amount) of sequences at each of the panel of condition-associated genomic loci in the sample. Assay readouts may comprise quantitative PCR (qPCR) values, digital PCR (dPCR) values, digital droplet PCR (ddPCR) values, fluorescence values, etc., or normalized values thereof.

Analysis of Single Nucleotide Polymorphisms (SNPs) Associated with Lupus

The present disclosure provides systems and methods to perform data analysis using drug or target scoring algorithms and/or big data analysis tools. In various aspects, such drug or target scoring algorithms and/or big data analysis tools may be used to perform analysis of data sets including, for example, mRNA gene expression or transcriptome data, DNA genomic data, proteomic data, metabolomic data, other types of “-omic” data, or a combination thereof.

In an aspect, the present disclosure provides a method for identifying an autoimmune disease drug target, the method comprising: (a) treating an autoimmune disease animal model with a drug configured to inhibit a drug target of the autoimmune disease, thereby producing a treated animal model; (b) assaying an animal biological sample of the treated animal model to obtain gene expression data of the treated animal model; (c) processing the gene expression data to obtain an animal gene signature, wherein the animal gene signature comprises quantitative measures of a first set of genomic loci associated with autoimmune disease pathways of the autoimmune disease animal model; (d) obtaining a set of human gene signatures, wherein the set of human gene signatures comprises quantitative measures of a second set of genomic loci associated with up-regulation or down-regulation of human autoimmune disease pathways in human patients having active autoimmune disease, and wherein the set of human gene signatures is generated by assaying human biological samples from one or more human patients having the autoimmune disease to obtain gene expression data; (e) processing the animal gene signature with the set of human gene signatures to identify (i) an animal genomic locus from among the first set of genomic loci, and (ii) a human genomic locus from among the second set of genomic loci that is associated with up-regulation or down-regulation of one or more human autoimmune disease pathways, wherein the animal genomic locus and the human genomic locus are orthologous and share similarity in expression patterns and function; and (f) identifying the drug target as the autoimmune disease drug target when the quantitative measure of the animal genomic locus of the animal gene signature is indicative of up-regulation or down-regulation of an autoimmune disease pathway of the autoimmune disease animal model.

In some embodiments, the autoimmune disease animal model is selected from: a mouse model, a rat model, a cat model, a dog model, a rabbit model, a guinea pig model, a hamster model, a pig model, a horse model, and a primate model. In some embodiments, the autoimmune disease animal model comprises a mouse model. In some embodiments, the autoimmune disease comprises lupus. In some embodiments, the lupus comprises systemic lupus erythematosus (SLE) or discoid lupus erythematosus (DLE). In some embodiments, the drug target is HDAC6. In some embodiments, the drug target is HDAC6 or a portion thereof. In some embodiments, the drug is an HDAC6 inhibitor. In some embodiments, the HDAC6 inhibitor is ACY-738. In some embodiments, the animal biological sample or the human biological samples comprise one or more of: a bodily fluid sample, a blood sample, a cell sample, and a tissue sample. In some embodiments, the one or more human autoimmune disease pathways are selected from the pathways listed in Table 61, Table 62, Table 63, and Table 64. In some embodiments, the human genomic locus that is associated with up-regulation or down-regulation of the one or more human autoimmune disease pathways is selected from the genes listed in Table 59, Table 60, Table 61, Table 62, Table 63, Table 64, Table 65, Table 66, Table 67, Table 68, and Table 69. In some embodiments, the autoimmune disease pathways of the autoimmune disease animal model are selected from the pathways listed in Table 61, Table 62, Table 63, and Table 64. In some embodiments, the animal genomic locus is selected from the genes listed in Table 59, Table 60, Table 61, Table 62, Table 63, Table 64, Table 65, Table 66, Table 67, Table 68, and Table 69. In some embodiments, (e) comprises identifying (i) a plurality of animal genomic loci from among the first set of genomic loci, and (ii) a plurality of human genomic loci from among the second set of genomic loci that is associated with up-regulation or down-regulation of a plurality of human autoimmune disease pathways, wherein plurality of animal genomic loci and the plurality of human genomic loci are pairwise orthologous and share similarities in expression patterns and function; and (f) comprises identifying the drug target as the autoimmune disease drug target when the quantitative measures of the plurality of animal genomic loci of the animal gene signature are indicative of up-regulation or down-regulation of a plurality of autoimmune disease pathways of the autoimmune disease animal model. In some embodiments, the plurality of human autoimmune disease pathways comprises between 2 and 5 different human autoimmune disease pathways. In some embodiments, the plurality of human autoimmune disease pathways comprises 1, 2, 3, 4, 5, 6, 7, 8, 9, 10, 11, 12, 13, 14, 15, 16, 17, 18, 19, 20, about 25, about 30, about 35, about 40, about 45, about 50, about 60, about 70, about 80, about 90, about 100, or more than about 100 different human autoimmune disease pathways. In some embodiments, the autoimmune disease pathways of the autoimmune disease animal model comprises 1, 2, 3, 4, 5, 6, 7, 8, 9, 10, 11, 12, 13, 14, 15, 16, 17, 18, 19, 20, about 25, about 30, about 35, about 40, about 45, about 50, about 60, about 70, about 80, about 90, about 100, or more than about 100 different autoimmune disease pathways. In some embodiments, the method further comprises determining the up-regulation or down-regulation of the autoimmune disease pathway of the autoimmune disease animal model based on determining a difference between the quantitative measure of the animal genomic locus of the animal gene signature and a reference quantitative measure of the animal genomic locus. In some embodiments, the method further comprises obtaining the reference quantitative measure of the animal genomic locus by, prior to (a), assaying an animal biological sample of the autoimmune disease animal model.

In another aspect, the present disclosure provides a computer-implemented method for identifying an autoimmune disease drug target, the method comprising: (a) obtaining gene expression data generated by assaying an animal biological sample of a treated animal model, wherein the treated animal model is obtained by treating an autoimmune disease animal model with a drug configured to inhibit a drug target of the autoimmune disease; (b) processing the gene expression data to obtain an animal gene signature, wherein the animal gene signature comprises quantitative measures of a first set of genomic loci associated with autoimmune disease pathways of the autoimmune disease animal model; (c) obtaining a set of human gene signatures, wherein the set of human gene signatures comprises quantitative measures of a second set of genomic loci associated with up-regulation or down-regulation of human autoimmune disease pathways in human patients having active autoimmune disease, and wherein the set of human gene signatures is generated by assaying human biological samples from one or more human patients having the autoimmune disease to obtain gene expression data; (d) processing the animal gene signature with the set of human gene signatures to identify (i) an animal genomic locus from among the first set of genomic loci, and (ii) a human genomic locus from among the second set of genomic loci that is associated with up-regulation or down-regulation of one or more human autoimmune disease pathways, wherein the animal genomic locus and the human genomic locus are orthologous and share similarity in expression patterns and function; and (e) identifying the drug target as the autoimmune disease drug target when the quantitative measure of the animal genomic locus of the animal gene signature is indicative of up-regulation or down-regulation of an autoimmune disease pathway of the autoimmune disease animal model.

In some embodiments, the autoimmune disease animal model is selected from: a mouse model, a rat model, a cat model, a dog model, a rabbit model, a guinea pig model, a hamster model, a pig model, a horse model, and a primate model. In some embodiments, the autoimmune disease animal model comprises a mouse model. In some embodiments, the autoimmune disease comprises lupus. In some embodiments, the lupus comprises systemic lupus erythematosus (SLE) or discoid lupus erythematosus (DLE). In some embodiments, the drug target is HDAC6. In some embodiments, the drug target is HDAC6 or a portion thereof. In some embodiments, the drug is an HDAC6 inhibitor. In some embodiments, the HDAC6 inhibitor is ACY-738. In some embodiments, the animal biological sample or the human biological samples comprise one or more of: a bodily fluid sample, a blood sample, a cell sample, and a tissue sample. In some embodiments, the one or more human autoimmune disease pathways are selected from the pathways listed in Table 61, Table 62, Table 63, and Table 64. In some embodiments, the human genomic locus that is associated with up-regulation or down-regulation of the one or more human autoimmune disease pathways is selected from the genes listed in Table 59, Table 60, Table 61, Table 62, Table 63, Table 64, Table 65, Table 66, Table 67, Table 68, and Table 69. In some embodiments, the autoimmune disease pathways of the autoimmune disease animal model are selected from the pathways listed in Table 61, Table 62, Table 63, and Table 64. In some embodiments, the animal genomic locus is selected from the genes listed in Table 59, Table 60, Table 61, Table 62, Table 63, Table 64, Table 65, Table 66, Table 67, Table 68, and Table 69. In some embodiments, (d) comprises identifying (i) a plurality of animal genomic loci from among the first set of genomic loci, and (ii) a plurality of human genomic loci from among the second set of genomic loci that is associated with up-regulation or down-regulation of a plurality of human autoimmune disease pathways, wherein plurality of animal genomic loci and the plurality of human genomic loci are pairwise orthologous and share similarities in expression patterns and function; and (e) comprises identifying the drug target as the autoimmune disease drug target when the quantitative measures of the plurality of animal genomic loci of the animal gene signature are indicative of up-regulation or down-regulation of a plurality of autoimmune disease pathways of the autoimmune disease animal model. In some embodiments, the plurality of human autoimmune disease pathways comprises between 2 and 5 different human autoimmune disease pathways. In some embodiments, the plurality of human autoimmune disease pathways comprises 1, 2, 3, 4, 5, 6, 7, 8, 9, 10, 11, 12, 13, 14, 15, 16, 17, 18, 19, 20, about 25, about 30, about 35, about 40, about 45, about 50, about 60, about 70, about 80, about 90, about 100, or more than about 100 different human autoimmune disease pathways. In some embodiments, the autoimmune disease pathways of the autoimmune disease animal model comprises 1, 2, 3, 4, 5, 6, 7, 8, 9, 10, 11, 12, 13, 14, 15, 16, 17, 18, 19, 20, about 25, about 30, about 35, about 40, about 45, about 50, about 60, about 70, about 80, about 90, about 100, or more than about 100 different autoimmune disease pathways. In some embodiments, the method further comprises determining the up-regulation or down-regulation of the autoimmune disease pathway of the autoimmune disease animal model based on determining a difference between the quantitative measure of the animal genomic locus of the animal gene signature and a reference quantitative measure of the animal genomic locus. In some embodiments, the method further comprises obtaining the reference quantitative measure of the animal genomic locus by, prior to (a), assaying an animal biological sample of the autoimmune disease animal model.

In another aspect, the present disclosure provides a computer system for identifying an autoimmune disease drug target, comprising: a database that is configured to store gene expression data generated by assaying an animal biological sample of a treated animal model, wherein the treated animal model is obtained by treating an autoimmune disease animal model with a drug configured to inhibit a drug target of the autoimmune disease; and one or more computer processors operatively coupled to the database, wherein the one or more computer processors are individually or collectively programmed to: (i) process the transcriptomic data to obtain an animal gene signature, wherein the animal gene signature comprises quantitative measures of a first set of genomic loci associated with autoimmune disease pathways of the autoimmune disease animal model; (ii) obtain a set of human gene signatures, wherein the set of human gene signatures comprises quantitative measures of a second set of genomic loci associated with up-regulation or down-regulation of human autoimmune disease pathways in human patients having active autoimmune disease, and wherein the set of human gene signatures is generated by assaying human biological samples from one or more human patients having the autoimmune disease to obtain gene expression data; (iii) process the animal gene signature with the set of human gene signatures to identify (1) an animal genomic locus from among the first set of genomic loci, and (2) a human genomic locus from among the second set of genomic loci that is associated with up-regulation or down-regulation of one or more human autoimmune disease pathways, wherein the animal genomic locus and the human genomic locus are orthologous and share similarity in expression patterns and function; and (iv) identify the drug target as the autoimmune disease drug target when the quantitative measure of the animal genomic locus of the animal gene signature is indicative of up-regulation or down-regulation of an autoimmune disease pathway of the autoimmune disease animal model.

In some embodiments, the autoimmune disease animal model is selected from: a mouse model, a rat model, a cat model, a dog model, a rabbit model, a guinea pig model, a hamster model, a pig model, a horse model, and a primate model. In some embodiments, the autoimmune disease animal model comprises a mouse model. In some embodiments, the autoimmune disease comprises lupus. In some embodiments, the lupus comprises systemic lupus erythematosus (SLE) or discoid lupus erythematosus (DLE). In some embodiments, the drug target is HDAC6. In some embodiments, the drug target is HDAC6 or a portion thereof. In some embodiments, the drug is an HDAC6 inhibitor. In some embodiments, the HDAC6 inhibitor is ACY-738. In some embodiments, the animal biological sample or the human biological samples comprise one or more of: a bodily fluid sample, a blood sample, a cell sample, and a tissue sample. In some embodiments, the one or more human autoimmune disease pathways are selected from the pathways listed in Table 61, Table 62, Table 63, and Table 64. In some embodiments, the human genomic locus that is associated with up-regulation or down-regulation of the one or more human autoimmune disease pathways is selected from the genes listed in Table 59, Table 60, Table 61, Table 62, Table 63, Table 64, Table 65, Table 66, Table 67, Table 68, and Table 69. In some embodiments, the autoimmune disease pathways of the autoimmune disease animal model are selected from the pathways listed in Table 61, Table 62, Table 63, and Table 64. In some embodiments, the animal genomic locus is selected from the genes listed in Table 59, Table 60, Table 61, Table 62, Table 63, Table 64, Table 65, Table 66, Table 67, Table 68, and Table 69. In some embodiments, (iii) comprises identifying (1) a plurality of animal genomic loci from among the first set of genomic loci, and (2) a plurality of human genomic loci from among the second set of genomic loci that is associated with up-regulation or down-regulation of a plurality of human autoimmune disease pathways, wherein plurality of animal genomic loci and the plurality of human genomic loci are pairwise orthologous and share similarities in expression patterns and function; and (iv) comprises identifying the drug target as the autoimmune disease drug target when the quantitative measures of the plurality of animal genomic loci of the animal gene signature are indicative of up-regulation or down-regulation of a plurality of autoimmune disease pathways of the autoimmune disease animal model. In some embodiments, the plurality of human autoimmune disease pathways comprises between 2 and 5 different human autoimmune disease pathways. In some embodiments, the plurality of human autoimmune disease pathways comprises 1, 2, 3, 4, 5, 6, 7, 8, 9, 10, 11, 12, 13, 14, 15, 16, 17, 18, 19, 20, about 25, about 30, about 35, about 40, about 45, about 50, about 60, about 70, about 80, about 90, about 100, or more than about 100 different human autoimmune disease pathways. In some embodiments, the autoimmune disease pathways of the autoimmune disease animal model comprises 1, 2, 3, 4, 5, 6, 7, 8, 9, 10, 11, 12, 13, 14, 15, 16, 17, 18, 19, 20, about 25, about 30, about 35, about 40, about 45, about 50, about 60, about 70, about 80, about 90, about 100, or more than about 100 different autoimmune disease pathways. In some embodiments, the one or more computer processors are individually or collectively programmed to further determine the up-regulation or down-regulation of the autoimmune disease pathway of the autoimmune disease animal model based on determining a difference between the quantitative measure of the animal genomic locus of the animal gene signature and a reference quantitative measure of the animal genomic locus. In some embodiments, the one or more computer processors are individually or collectively programmed to further obtain the reference quantitative measure of the animal genomic locus by, prior to (a), assaying an animal biological sample of the autoimmune disease animal model.

In another aspect, the present disclosure provides a non-transitory computer readable medium comprising machine-executable code that, upon execution by one or more computer processors, implements a method for identifying an autoimmune disease drug target, the method comprising: (a) obtaining gene expression data generated by assaying an animal biological sample of a treated animal model, wherein the treated animal model is obtained by treating an autoimmune disease animal model with a drug configured to inhibit a drug target of the autoimmune disease; (b) processing the gene expression data to obtain an animal gene signature, wherein the animal gene signature comprises quantitative measures of a first set of genomic loci associated with autoimmune disease pathways of the autoimmune disease animal model; (c) obtaining a set of human gene signatures, wherein the set of human gene signatures comprises quantitative measures of a second set of genomic loci associated with up-regulation or down-regulation of human autoimmune disease pathways in human patients having active autoimmune disease, and wherein the set of human gene signatures is generated by assaying human biological samples from one or more human patients having the autoimmune disease to obtain gene expression data; (d) processing the animal gene signature with the set of human gene signatures to identify (i) an animal genomic locus from among the first set of genomic loci, and (ii) a human genomic locus from among the second set of genomic loci that is associated with up-regulation or down-regulation of one or more human autoimmune disease pathways, wherein the animal genomic locus and the human genomic locus are orthologous and share similarity in expression patterns and function; and (e) identifying the drug target as the autoimmune disease drug target when the quantitative measure of the animal genomic locus of the animal gene signature is indicative of up-regulation or down-regulation of an autoimmune disease pathway of the autoimmune disease animal model.

In some embodiments, the autoimmune disease animal model is selected from: a mouse model, a rat model, a cat model, a dog model, a rabbit model, a guinea pig model, a hamster model, a pig model, a horse model, and a primate model. In some embodiments, the autoimmune disease animal model comprises a mouse model. In some embodiments, the autoimmune disease comprises lupus. In some embodiments, the lupus comprises systemic lupus erythematosus (SLE) or discoid lupus erythematosus (DLE). In some embodiments, the drug target is HDAC6. In some embodiments, the drug target is HDAC6 or a portion thereof. In some embodiments, the drug is an HDAC6 inhibitor. In some embodiments, the HDAC6 inhibitor is ACY-738. In some embodiments, the animal biological sample or the human biological samples comprise one or more of: a bodily fluid sample, a blood sample, a cell sample, and a tissue sample. In some embodiments, the one or more human autoimmune disease pathways are selected from the pathways listed in Table 61, Table 62, Table 63, and Table 64. In some embodiments, the human genomic locus that is associated with up-regulation or down-regulation of the one or more human autoimmune disease pathways is selected from the genes listed in Table 59, Table 60, Table 61, Table 62, Table 63, Table 64, Table 65, Table 66, Table 67, Table 68, and Table 69. In some embodiments, the autoimmune disease pathways of the autoimmune disease animal model are selected from the pathways listed in Table 61, Table 62, Table 63, and Table 64. In some embodiments, the animal genomic locus is selected from the genes listed in Table 59, Table 60, Table 61, Table 62, Table 63, Table 64, Table 65, Table 66, Table 67, Table 68, and Table 69. In some embodiments, (d) comprises identifying (i) a plurality of animal genomic loci from among the first set of genomic loci, and (ii) a plurality of human genomic loci from among the second set of genomic loci that is associated with up-regulation or down-regulation of a plurality of human autoimmune disease pathways, wherein plurality of animal genomic loci and the plurality of human genomic loci are pairwise orthologous and share similarities in expression patterns and function; and (e) comprises identifying the drug target as the autoimmune disease drug target when the quantitative measures of the plurality of animal genomic loci of the animal gene signature are indicative of up-regulation or down-regulation of a plurality of autoimmune disease pathways of the autoimmune disease animal model. In some embodiments, the plurality of human autoimmune disease pathways comprises between 2 and 5 different human autoimmune disease pathways. In some embodiments, the plurality of human autoimmune disease pathways comprises 1, 2, 3, 4, 5, 6, 7, 8, 9, 10, 11, 12, 13, 14, 15, 16, 17, 18, 19, 20, about 25, about 30, about 35, about 40, about 45, about 50, about 60, about 70, about 80, about 90, about 100, or more than about 100 different human autoimmune disease pathways. In some embodiments, the autoimmune disease pathways of the autoimmune disease animal model comprises 1, 2, 3, 4, 5, 6, 7, 8, 9, 10, 11, 12, 13, 14, 15, 16, 17, 18, 19, 20, about 25, about 30, about 35, about 40, about 45, about 50, about 60, about 70, about 80, about 90, about 100, or more than about 100 different autoimmune disease pathways. In some embodiments, the method further comprises determining the up-regulation or down-regulation of the autoimmune disease pathway of the autoimmune disease animal model based on determining a difference between the quantitative measure of the animal genomic locus of the animal gene signature and a reference quantitative measure of the animal genomic locus. In some embodiments, the method further comprises obtaining the reference quantitative measure of the animal genomic locus by, prior to (a), assaying an animal biological sample of the autoimmune disease animal model.

In another aspect, the present disclosure provides a method for evaluating a drug candidate for an autoimmune disease, the method comprising: (a) treating an autoimmune disease animal model with the drug candidate for the autoimmune disease, thereby producing a treated animal model; (b) assaying an animal biological sample of the treated animal model to obtain gene expression data of the treated animal model; (c) processing the gene expression data to obtain an animal gene signature, wherein the animal gene signature comprises quantitative measures of a first set of genomic loci associated with autoimmune disease pathways of the autoimmune disease animal model; (d) obtaining a set of human gene signatures, wherein the set of human gene signatures comprises quantitative measures of a second set of genomic loci associated with up-regulation or down-regulation of human autoimmune disease pathways in human patients having active autoimmune disease, and wherein the set of human gene signatures is generated by assaying human biological samples from one or more human patients having the autoimmune disease to obtain gene expression data; (e) processing the animal gene signature with the set of human gene signatures to identify (i) an animal genomic locus from among the first set of genomic loci, and (ii) a human genomic locus from among the second set of genomic loci that is associated with up-regulation or down-regulation of one or more human autoimmune disease pathways, wherein the animal genomic locus and the human genomic locus are orthologous and share similarity in expression patterns and function; and (f) evaluating the efficacy of the drug candidate for the autoimmune disease based at least in part on whether the quantitative measure of the animal genomic locus of the animal gene signature is indicative of up-regulation or down-regulation of an autoimmune disease pathway of the autoimmune disease animal model.

In another aspect, the present disclosure provides a computer-implemented method for evaluating a drug candidate for an autoimmune disease, the method comprising: (a) obtaining gene expression data generated by assaying an animal biological sample of a treated animal model, wherein the treated animal model is obtained by treating an autoimmune disease animal model with the drug candidate for the autoimmune disease; (b) processing the gene expression data to obtain an animal gene signature, wherein the animal gene signature comprises quantitative measures of a first set of genomic loci associated with autoimmune disease pathways of the autoimmune disease animal model; (c) obtaining a set of human gene signatures, wherein the set of human gene signatures comprises quantitative measures of a second set of genomic loci associated with up-regulation or down-regulation of human autoimmune disease pathways in human patients having active autoimmune disease, and wherein the set of human gene signatures is generated by assaying human biological samples from one or more human patients having the autoimmune disease to obtain gene expression data; (d) processing the animal gene signature with the set of human gene signatures to identify (i) an animal genomic locus from among the first set of genomic loci, and (ii) a human genomic locus from among the second set of genomic loci that is associated with up-regulation or down-regulation of one or more human autoimmune disease pathways, wherein the animal genomic locus and the human genomic locus are orthologous and share similarity in expression patterns and function; and (e) evaluating the efficacy of the drug candidate for the autoimmune disease based at least in part on whether the quantitative measure of the animal genomic locus of the animal gene signature is indicative of up-regulation or down-regulation of an autoimmune disease pathway of the autoimmune disease animal model.

In another aspect, the present disclosure provides a computer system for evaluating a drug candidate for an autoimmune disease, comprising: a database that is configured to store gene expression data generated by assaying an animal biological sample of a treated animal model, wherein the treated animal model is obtained by treating an autoimmune disease animal model with the drug candidate for the autoimmune disease; and one or more computer processors operatively coupled to the database, wherein the one or more computer processors are individually or collectively programmed to: (i) process the transcriptomic data to obtain an animal gene signature, wherein the animal gene signature comprises quantitative measures of a first set of genomic loci associated with autoimmune disease pathways of the autoimmune disease animal model; (ii) obtain a set of human gene signatures, wherein the set of human gene signatures comprises quantitative measures of a second set of genomic loci associated with up-regulation or down-regulation of human autoimmune disease pathways in human patients having active autoimmune disease, and wherein the set of human gene signatures is generated by assaying human biological samples from one or more human patients having the autoimmune disease to obtain gene expression data; (iii) process the animal gene signature with the set of human gene signatures to identify (1) an animal genomic locus from among the first set of genomic loci, and (2) a human genomic locus from among the second set of genomic loci that is associated with up-regulation or down-regulation of one or more human autoimmune disease pathways, wherein the animal genomic locus and the human genomic locus are orthologous and share similarity in expression patterns and function; and (iv) evaluate the efficacy of the drug candidate for the autoimmune disease based at least in part on whether the quantitative measure of the animal genomic locus of the animal gene signature is indicative of up-regulation or down-regulation of an autoimmune disease pathway of the autoimmune disease animal model.

In another aspect, the present disclosure provides a non-transitory computer readable medium comprising machine-executable code that, upon execution by one or more computer processors, implements a method for evaluating a drug candidate for an autoimmune disease, the method comprising: (a) treating an autoimmune disease animal model with the drug candidate for the autoimmune disease, thereby producing a treated animal model; (b) assaying an animal biological sample of the treated animal model to obtain gene expression data of the treated animal model; (c) processing the gene expression data to obtain an animal gene signature, wherein the animal gene signature comprises quantitative measures of a first set of genomic loci associated with autoimmune disease pathways of the autoimmune disease animal model; (d) obtaining a set of human gene signatures, wherein the set of human gene signatures comprises quantitative measures of a second set of genomic loci associated with up-regulation or down-regulation of human autoimmune disease pathways in human patients having active autoimmune disease, and wherein the set of human gene signatures is generated by assaying human biological samples from one or more human patients having the autoimmune disease to obtain gene expression data; (e) processing the animal gene signature with the set of human gene signatures to identify (i) an animal genomic locus from among the first set of genomic loci, and (ii) a human genomic locus from among the second set of genomic loci that is associated with up-regulation or down-regulation of one or more human autoimmune disease pathways, wherein the animal genomic locus and the human genomic locus are orthologous and share similarity in expression patterns and function; and (f) evaluating the efficacy of the drug candidate for the autoimmune disease based at least in part on whether the quantitative measure of the animal genomic locus of the animal gene signature is indicative of up-regulation or down-regulation of an autoimmune disease pathway of the autoimmune disease animal model.

To obtain a blood sample, various techniques may be used, e.g., a syringe or other vacuum suction device. A blood sample may be optionally pre-treated or processed prior to use. A sample, such as a blood sample, may be analyzed under any of the methods and systems herein within 4 weeks, 2 weeks, 1 week, 6 days, 5 days, 4 days, 3 days, 2 days, 1 day, 12 hr, 6 hr, 3 hr, 2 hr, or 1 hr from the time the sample is obtained, or longer if frozen. When obtaining a sample from a subject (e.g., blood sample), the amount may vary depending upon subject size and the condition being screened. In some embodiments, at least 10 mL, 5 mL, 1 mL, 0.5 mL, 250, 200, 150, 100, 50, 40, 30, 20, 10, 9, 8, 7, 6, 5, 4, 3, 2, or 1 μL of a sample is obtained. In some embodiments, 1-50, 2-40, 3-30, or 4-20 μL of sample is obtained. In some embodiments, more than 5, 10, 15, 20, 25, 30, 35, 40, 45, 50, 55, 60, 65, 70, 75, 80, 85, 90, 95 or 100 μL of a sample is obtained.

The sample may be taken before and/or after treatment of a subject with a disease or disorder. Samples may be obtained from a subject during a treatment or a treatment regime. Multiple samples may be obtained from a subject to monitor the effects of the treatment over time. The sample may be taken from a subject known or suspected of having a disease or disorder for which a definitive positive or negative diagnosis is not available via clinical tests. The sample may be taken from a subject suspected of having a disease or disorder. The sample may be taken from a subject experiencing unexplained symptoms, such as fatigue, nausea, weight loss, aches and pains, weakness, or bleeding. The sample may be taken from a subject having explained symptoms. The sample may be taken from a subject at risk of developing a disease or disorder due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

In some embodiments, a sample may be taken at a first time point and assayed, and then another sample may be taken at a subsequent time point and assayed. Such methods may be used, for example, for longitudinal monitoring purposes to track the development or progression of a disease. In some embodiments, the progression of a disease may be tracked before treatment, after treatment, or during the course of treatment, to determine the treatment's effectiveness. For example, a method as described herein may be performed on a subject prior to, and after, treatment with a lupus condition therapy to measure the disease's progression or regression in response to the lupus condition therapy.

After obtaining a sample from the subject, the sample may be processed or assayed to generate datasets of the subject. The datasets may be indicative of a disease, disorder, or abnormal condition (e.g., lupus) of the subject. For example, a presence, absence, or quantitative assessment of nucleic acid molecules of the sample at a panel of condition-associated genomic loci may comprise a gene signature of a subject (e.g., a mouse or human). The gene signature may be indicative of a autoimmune disease (e.g., lupus) of the subject or of suitable disease targets of the autoimmune disease. Processing or assaying the sample obtained from the subject may comprise (i) subjecting the sample to conditions that are sufficient to isolate, enrich, or extract a plurality of nucleic acid molecules, and (ii) assaying the plurality of nucleic acid molecules to generate the dataset (e.g., microarray data, nucleic acid sequences, or quantitative polymerase chain reaction (qPCR) data). Methods of assaying may include the use of a variety of suitable assays, for example, a microarray assay, a sequencing assay (e.g., DNA sequencing, RNA sequencing, or RNA-Seq), a single-cell assay, or a quantitative polymerase chain reaction (qPCR) assay.

In some embodiments, single-cell RNA-Seq data may be obtained from biological samples and then analyzed by a clustering approach such as spherical transformation and recursive splitting for heuristic identification of partitions (Starship), which is adapted for single-cell RNA-Seq data. Generally, bulk cell analysis methods may fail to account for the zero-inflated nature of single-cell RNA-Seq data. For example, Euclidean-based methods may be confounded by the vast number of zeros, which tends to make all cells look similar. In addition, density-based methods may fail to adapt to different levels of heterogeneity among leukocytes (e.g., the differences between myeloid populations may be more prominent than those between B cells and T cells). For example, conventional methods may be unable to cluster all of the cells in one pass, and may need to be re-run manually on sub-clusters to fully partition the cells. Single-cell RNA-Seq data, particularly those gathered with Unique Molecular Identifier (UMI) barcodes, may tend to resemble bag-of-words text data in several ways, such as: 1) each observation takes an integer value, and 2) most genes may not appear in a given cell, much like most words may not appear in a given document. Clustering of this sparse data may be performed by mapping samples onto the surface of a unit n-dimensional sphere, where n is the number of genes. Rather than clustering with a set number of clusters (k), Starship recursively clusters data with k=2 until pre-defined stop criteria are met. Once the clustering is complete, several functions can be run to further analyze and/or visualize the resulting clusters of cells. The Starship algorithm may be performed as described in, for example, PCT Appl. No. PCT/US2019/049129, entitled “Systems and Methods for Single-Cell RNA-Seq Data Analysis,” filed Aug. 30, 2019, which is incorporated herein by reference in its entirety.

In some embodiments, a plurality of nucleic acid molecules is extracted from the sample and subjected to sequencing to generate a plurality of sequencing reads. The nucleic acid molecules may comprise ribonucleic acid (RNA) or deoxyribonucleic acid (DNA). The extraction method may extract all RNA or DNA molecules from a sample. Alternatively, the extraction method may selectively extract a portion of RNA or DNA molecules from a sample. Extracted RNA molecules from a sample may be converted to cDNA molecules by reverse transcription (RT).

The sample may be processed without any nucleic acid extraction. For example, the disease, disorder, or abnormal condition (e.g., an autoimmune disease such as lupus) may be identified or monitored in the subject by using probes configured to selectively enrich nucleic acid (e.g., RNA or DNA) molecules corresponding to a panel of condition-associated genomic loci. The probes may be nucleic acid primers. The probes may have sequence complementarity with nucleic acid sequences from one or more of the panel of condition-associated genomic loci. The panel of condition-associated genomic loci may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more condition-associated genomic loci.

The probes may be nucleic acid molecules (e.g., RNA or DNA) having sequence complementarity with nucleic acid sequences (e.g., RNA or DNA) of one or more genomic loci (e.g., condition-associated genomic loci). These nucleic acid molecules may be primers or enrichment sequences. The assaying of the sample using probes that are selective for the one or more genomic loci (e.g., condition-associated genomic loci) may comprise use of array hybridization, polymerase chain reaction (PCR), or nucleic acid sequencing (e.g., RNA sequencing or DNA sequencing, such as RNA-Seq).

The assay readouts may be quantified at one or more genomic loci (e.g., condition-associated genomic loci) to generate the data indicative of the disease or disorder. For example, quantification of array hybridization or polymerase chain reaction (PCR) corresponding to a plurality of genomic loci (e.g., condition-associated genomic loci) may generate data indicative of the disease or disorder. Assay readouts may comprise quantitative PCR (qPCR) values, digital PCR (dPCR) values, digital droplet PCR (ddPCR) values, fluorescence values, etc., or normalized values thereof.

### Data Analysis

The present disclosure provides systems and methods to identify autoimmune disease drug targets using data analysis tools or algorithms. In various aspects, such data analysis tools or algorithms may be used to perform analysis of data sets including, for example, mRNA gene expression or transcriptome data, DNA genomic data, proteomic data, metabolomic data, other types of “-omic” data, or a combination thereof. Methods and systems of the present disclosure may use one or more of the following: a BIG-C™ data analysis algorithm, an I-Scope™ data analysis algorithm, a T-Scope™ data analysis algorithm, a P-Scope™ data analysis algorithm, and a Gene Set Variation Analysis (GSVA) algorithm.

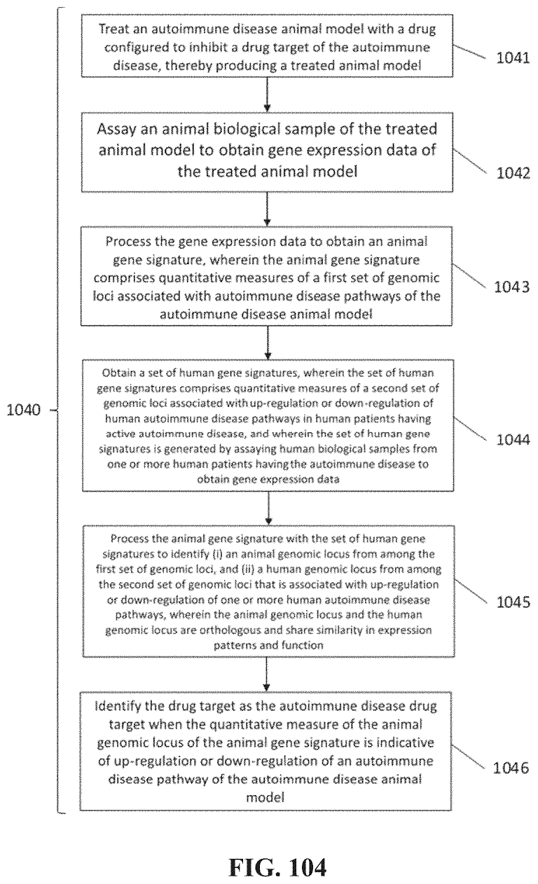

FIG. 104 shows a non-limiting example of a workflow of a method 1040 to identify an autoimmune disease drug target, using one or more data analysis algorithms or tools. The method may comprise treating an autoimmune disease animal model with a drug configured to inhibit a drug target of the autoimmune disease, thereby producing a treated animal model (as in operation 1041). Next, the method may comprise assaying an animal biological sample of the treated animal model to obtain gene expression data of the treated animal model (as in operation 1042). Next, the method may comprise processing the gene expression data to obtain an animal gene signature (as in operation 1043). In some embodiments, the animal gene signature comprises quantitative measures of a first set of genomic loci associated with autoimmune disease pathways of the autoimmune disease animal model. Next, the method may comprise obtaining a set of human gene signatures (as in 1044). In some embodiments, the set of human gene signatures comprises quantitative measures of a second set of genomic loci associated with up-regulation or down-regulation of human autoimmune disease pathways in human patients having active autoimmune disease. In some embodiments, the set of human gene signatures is generated by assaying human biological samples from one or more human patients having the autoimmune disease to obtain gene expression data. Next, the method may comprise processing the animal gene signature with the set of human gene signatures to identify (i) an animal genomic locus from among the first set of genomic loci, and (ii) a human genomic locus from among the second set of genomic loci that is associated with up-regulation or down-regulation of one or more human autoimmune disease pathways (as in operation 1045). In some embodiments, the animal genomic locus and the human genomic locus are orthologous and share similarity in expression patterns and function. Next, the method may comprise identifying the drug target as the autoimmune disease drug target based on the quantitative measure of the animal genomic locus of the animal gene signature (e.g., when the quantitative measure of the animal genomic locus of the animal gene signature is indicative of up-regulation or down-regulation of an autoimmune disease pathway of the autoimmune disease animal) model (as in operation 1046).

BIG-C™ Big Data Analysis Algorithm

BIG-C® may be a fast and efficient cloud-based algorithm to functionally categorize gene products. With coverage of over 80% of the genome, BIG-C® leverages publicly available databases such as UniProtKB/Swiss-Prot, GO terms, KEGG pathways, NCBI PubMed and Interactome to place genes into 53 functional categories. The sorting into only one of 53 functional groups allows for a quick and relatively simple understanding of types of genes enriched and co-expressed in a big dataset. This assists in deriving further insights from genes expressed for a given disease state in human or pre-clinical mouse models.

BIG-C® may be used to functionally categorize immunological genes that are not covered in cancer databases such as GO and KEGG (e.g., as described by Grammer et al. 2016, “Drug repositioning in SLE: crowd-sourcing, literature-mining and Big Data analysis,” Lupus, 25(10), 1150-1170, which is incorporated herein by reference in its entirety). Using a knowledge base of over 5000 patients with systemic lupus erythematosus (SLE), over 16432 genes are each placed into one of 53 BIG-C® functional categories, and statistical analysis is performed to identify enriched categories. BIG-C® categories are cross-examined with the GO and KEGG terms to obtain additional information and insights.

The BIG-C (Biologically Informed Gene Clustering) algorithm may be configured to sort large groups of genes into a set of functional groups (e.g., 53 functional groups). The functional groups are created utilizing publicly available information from online tools and databases including UniProtKB/Swiss-Prot, GO Terms, KEGG pathways, NCBI PubMed, and the Interactome. The functional groups may include one or more of: Active RNA, Anti-apoptosis, anti-proliferation, autophagy, chromatin remodeling, cytoplasm and biochemistry, cytoskeleton, DNA repair, endocytosis, endoplasmic reticulum, endosome and vesicles, fatty acid biosynthesis, cell surface, transcription, glycolysis and gluconeogenesis, golgi, immune cell surface, immune secreted, immune signaling, integrin pathway, interferon stimulated genes, intracellular signaling, lysosome, melanosome, MHC class I, MHC class II, microRNA processing, microRNA, mitochondrial transcription, mitochondria, mitochondria oxidative phosphorylation, mitochondrial TCA cycle, mRNA processing, mRNA splicing, non-coding RNA, nuclear receptor, nucleus and nucleolus, palmitoylation, pattern recognition receptors, peroxisomes, pro-apoptosis, pro-cell cycle, proteasome, pseudogenes, RAS superfamily, reactive oxygen species protection, secreted and extracellular matrix, transcription factors, transporters, transposon control, ubiquitylation and sumoylation, unfolded protein and stress, and unknown. Enrichment scores for each group are calculated based on an overlap p value to determine the functional groups over or under-expressed in the gene expression dataset. The BIG-C may be configured such that each gene is sorted into only one of the 53 functional groups, allowing for a quick and relatively simple understanding of types of genes enriched and co-expressed in a big dataset.

A sample BIG-C® workflow may comprise the following steps. First, SLE genomic datasets arederived from whole blood, peripheral blood mononuclear cells, affected tissues, and purified immune cells. Second, datasets are analyzed using differential expression analysis or Weighted Gene Coexpression Network Analysis (WGCNA). Third, expressed genes are annotated using publicly available databases (e.g., UniProtKB/Swiss-Prot database, Human Immunodeficiencies database, Mouse MGI database, Entrez Molecular Sequence database, PubMed, and the Human Tissue Atlas). Fourth, signatures are cross-referenced with purified single-cell microarray datasets and RNAseq experiments. Fifth, BIG-C® is leveraged to separate the individual annotated genes into one of 53 functional categories (e.g., as described by Labonte et al. 2018, “Identification of alterations in macrophage activation associated with disease activity in systemic lupus erythematosus,” PloS one, 13(12), e0208132, which is incorporated herein by reference in its entirety). Sixth, chi-squared analysis is used to determine enriched categories of interest from overlap p-values. Seventh, enriched categories are cross-examined with GO and KEGG terms to derive key insights for further analysis.

I-Scope™ Big Data Analysis

I-Scope™ may be a big data analysis algorithm configured for cross-examining the presence and activity of varying types of immune cell infiltrates with observed gene expression patterns. It may take annotated gene expression data and analyze it for hematopoietic cell lineage. I-Scope™ may be used downstream of the BIG-C® (Biologically Informed Gene-Clustering) tool in that it helps to provide even more insight into the nature of the genes being expressed after categorization.

I-Scope™ addresses the need to understand the involvement of specific cells for a given disease state. While it is helpful to understand the relative up-regulation and down-regulation at the gene expression level, it is even more informative to understand specifically in which cells this is occurring. I-Scope™ may be configured to identify hematopoietic cells through an iterative search of more than 17,000 genes identified in more than 50 microarray datasets (e.g., as described by Hubbard et al., “Analysis of Lupus Synovitis Gene Expression Reveals Dysregulation of Pathogenic Pathways Activated within Infiltrating Immune Cells,” Arthritis Rheumatol, 2018; 70 (suppl 10), which is incorporated herein by reference in its entirety). I-Scope™may function by restricting the analysis to genes of hematopoietic cell heritage and allow for cross-checking against purified single-cell experiments or datasets. The cross-check confirms and categorizes specific transcript signatures to the 28 hematopoietic cell sub-categories, ultimately allowing for cellular activity analysis across multiple samples and disease states. The hematopoietic cell sub-categories may include, for example, Monos/Macs, Plasma Cells, T-Cells, B-Cells, Dendritic, T&B Cells, CD8 T, Myeloid Cells, Tact, LDG, Hematopoietic, Neutrophil, Ag Presentation, Granulocytes, Platelets, pDC, “T, B, Mono”, Langerhans, Bact, Mono and B, Erythrocytes, Mast Cell, T reg, Gd T, T anergic, FDC, CD4T, and T/NK/NKT Cells. When combined with BIG-C® categories, the cellular activity may be correlated to specific functions within a given cell type.

The I-Scope™ algorithm may be configured to identify immune infiltrates. Hematopoietic cells are unique in that they move throughout the body patrolling for threats to the host, and may infiltrate tissue sites not normally home to immune cells. I-Scope™ may be configured to identify hematopoietic cells through an iterative search of more than 17,000 genes identified in more than 50 microarray datasets. From this search, 1226 candidate genes are identified and researched for restriction in hematopoietic cells as determined by the HPA, GTEx and FANTOMS datasets (e.g., available at proteinatlas.org). 926 genes meet the criteria for being mainly restricted to hematopoietic lineages (brain, reproductive organ exclusions were permitted). These genes are researched for immune cell specific expression in 27 hematopoietic sub-categories: alpha beta T cell, T cell, regulatory T Cell, activated T cell, anergic T cell, gamma delta T cells, CD8 T, NK/NKT cell, NK cell, T & B cells, B cells, germinal center B cells, B cell and plasmacytoid dendritic cell, T &B & myeloid, B & myeloid, T & myeloid, MHC Class II expressing cell, monocyte, dendritic cell, plasmacytoid dendritic cells, myeloid cell, plasma cell, erythrocyte, neutrophil, low density granulocyte, granulocyte, and platelet. Transcripts are entered into I-Scope™ and the number of transcripts in each category determined. Odd's ratios are calculated with confidence intervals using the Fisher's exact test in R.

A sample I-Scope™ workflow may comprise the following steps. First, candidate genes are identified from SLE (systemic lupus erythematosus) datasets potentially associated with immune cell expression. Second, using HPA, GTEx, and FANTOMS datasets, expression signatures associated with hematopoietic cell lineage are identified. Third, signatures are cross-referenced with purified single-cell microarray datasets and RNAseq experiments. Fourth, transcripts are categorized into 28 hematopoietic cell sub-categories and assess cellular expression across different samples and disease states. Odds ratios are calculated with confidence intervals using the Fisher's exact test in R. An I-Scope™ signature analysis for a given sample may generate an I-Scope™ signature analysis across multiple samples and disease states.

T-Scope™ Big Data Analysis

The T-Scope™ algorithm may be configured for cross-examining gene expression signatures of a given sample with a database of non-hematopoietic cell types (e.g., as described by Hubbard et al., “Analysis of Gene Expression from Systemic Lupus Erythematosus Synovium Reveals Unique Pathogenic Mechanisms [Abstract], Annual Meeting of the American College of Rheumatology; June 2019; Chicago, Ill., which is incorporated herein by reference in its entirety). T-Scope™ may comprise a database of 704 transcripts allocated to 45 independent categories. Transcripts detected in the sample are matched to one of the cellular categories within the T-Scope™ tool to derive further insights on tissue cell activity. T-Scope™ can be used downstream of the BIG-C® (Biologically Informed Gene-Clustering) tool to understand which tissue cell types are present. In conjunction with I-Scope™ (which provides information related to immune cells), T-Scope™ can be performed to provide a complete view of all possible cell activity in a given sample.

T-Scope™ addresses the need to understand the involvement of specific tissue cells for a given disease state. While it is helpful to understand the relative up-regulation and down-regulation at the gene expression level, it is even more informative to understand specifically in which cells this is occurring. T-Scope™ may be configured by downloading a set of approximately 10,000 tissue enriched and 8,000 cell line enriched genes from the Human Protein Atlas along with their tissue or cell line designation. Genes differentially expressed in hematopoietic cell datasets are removed and kidney specific genes are added from the GEO repository. T-Scope™ may function by restricting the analysis to genes of known tissue cell heritage and allow for cross-checking against purified single-cell experiments or datasets. The cross-check confirms and categorizes specific transcript signatures to the 45 tissue cell sub-categories (Adipose Tissue, Adrenal Gland, Breast, Cartilage, Cerebral Cortex, “Cervix, Uterine”, Chondrocyte, Colon, Dendritic, Duodenum, Endometrium, Endothelial, Epididymis, Erythrocytes, Esophagus, Fallopian Tube, Fibroblast, Gallbaldder, Heart Muscle, Keratinocyte, Keratinocyte Skin, Kidney, Kidney Distal Tubules, Kidney Loop, Kidney Proximal Tubules, Kidney Tubule Duct, Kidney Tubule, Langherhans, Liver, Lung, Melanocyte, Podocyte, Prostate, Rectum, Salivary Gland, Seminal Vesicle, Skeletal Muscle, Skin, Small Intenstine, Smooth Muscle, Stomach, Synoviocyte, Testis, Thyroid Gland, and Urinary Bladder), ultimately allowing for cellular activity analysis across multiple samples and disease states. When combined with BIG-C® categories, the cellular activity can be correlated to specific functions within a given tissue cell type.

The T-Scope™ algorithm may be configured to help identify types of non-hematopoietic cells in gene expression datasets. T-Scope™ may be configured by downloading approximately 10,000 tissue enriched and 8,000 cell line enriched genes from the human protein atlas along with their tissue or cell line designation (e.g., available at proteinatlas.org). Genes found in more than four tissues are eliminated. Housekeeping genes described in the gene expression study by She et al. are also removed (e.g., as described by She et al., “Definition, conservation and epigenetics of housekeeping and tissue-enriched genes,” BMC Genomics 2009, 10:269, which is incorporated herein by reference in its entirety). This list is further curated by removing genes differentially expressed in 34 hematopoietic cell gene expression datasets and adding kidney specific genes from datasets downloaded from the GEO repository and processed by Ampel BioSolutions. The resulting categories of genes represent genes enriched in the following 42 tissue or cell-specific categories: adrenal gland, breast, cartilage, cerebral cortex, uterine cervix, chondrocyte, colon, duodenum, endometrium, epididymis, esophagus fallopian tube, esophagus, fibroblast, heart muscle, keratinocyte, kidney, liver, lung, melanocyte, ovary pancreas, parathyroid gland, placenta, podocyte, prostrate, rectum, salivary gland, seminal vesicle, skeletal muscle, skin, small intestine, smooth muscle, stomach, synoviocyte, testis, kidney loop of henle, kidney proximal tubule, kidney distal tubule, and kidney collecting duct.

A sample T-Scope™ workflow may comprise the following steps. First, candidate genes are identified from SLE (systemic lupus erythematosus) differential expression datasets potentially associated with tissue cell expression. Second, using publicly available databases, expression signatures associated with potential tissue cell activity are identified. Third, signatures are cross-referenced with microarray, scRNAseq or RNAseq experiments. Fourth, transcripts are categorized into 45 tissue cell sub-categories and cellular expression is assessed across different samples and disease states. Using T-Scope™ in combination with I-Scope™ identification of cells post-DE-analysis may be performed.

Gene Set Variation Analysis (GSVA)

Gene Set Variation Analysis (GSVA) algorithms may be performed (for example, as described in Catalina et al. (2019, Communications Biology, “Gene expression analysis delineates the potential roles of multiple interferons in systemic lupus erythematosus”, which is incorporated herein by reference in its entirety) to determine enrichment of signaling pathways in individual patient samples. Gene set variation analysis may be performed using an open source software package for the coding language R available at the R Bioconductor (bioconductor.org), e.g., as described by Hanzelman et al., (“GSVA: gene set variation analysis for microarray and RNA-Seq data,” BMC Bioinformatics, 2013, which is incorporated herein by reference in its entirety). The modules of genes to interrogate the datasets may be developed. Modules of genes determined to represent a specific signaling pathway or process may be identified (e.g., using publicly available datasets). For example, the IFNB1 signaling pathway is taken from a publicly available gene expression dataset of peripheral blood cells treated with IFNB1 in vitro. Genes co-expressed in this dataset (genes either all increased or decreased compared to control treated peripheral blood) are used to create modules of genes representing the IFNB1 signaling pathway, and GSVA is used to determine the enrichment of this set of genes and hence the IFNB1 signaling pathway in individual patient and control samples.

A GSVA-based data analysis tool may be developed for use in analyzing specific sets of gene pathways. The GSVA-based data analysis tool (e.g., P-Scope) may use a GSVA statistical test-based tool using different sets of genes to analyze certain pathways. Such sets of genes may include, for example, human genes, mouse genes, or a combination thereof.

### EXAMPLES

The following illustrative examples are representative of embodiments of the software applications, systems, and methods described herein and are not meant to be limiting in any way.

### Example 1: Identification of Active Vs. Inactive SLE by Applying a Random Forest Classifier to SLE Gene Expression Data

Random forest, a high-performing classifier, may be used to perform analysis to sort through the inherent heterogeneity in raw SLE gene expression data and may be able to identify records with active versus inactive disease with a sensitivity of 85 percent and a specificity of 83 percent. Fine tuning the algorithms may be able to generate sufficient accuracy to be informative as a stand-alone estimate of disease activity. Accuracy may be assessed as the proportion of patients correctly classified across all testing folds.

SLE is a complex, multisystem autoimmune disease that continues to be a major diagnostic as well as therapeutic challenge. There are no definitive diagnostic tools available to determine whether a patient has SLE, and diagnostic approaches in SLE have not changed in decades. Physicians still rely on clinical evaluation and a few laboratory tests, including measurement of autoantibodies and complement levels. Despite the wealth of genetic, epigenetic, and gene expression data that has emerged in the past few years at both the patient and cellular levels, none has been integrated to produce a predictive tool that can be used to evaluate an individual SLE patient.

In SLE, defects in central and peripheral tolerance allow for activation of self-reactive B cell clones and differentiation into plasmablasts/plasma cells (PCs) that secrete autoantibodies, which in turn mediate tissue damage. Genome wide association studies (GWAS) have identified numerous polymorphisms in regions encoding genes or regulatory regions that may influence B cell function, suggesting that a general state of B cell hyper-responsiveness may contribute to SLE pathogenesis. Autoantibody-containing immune complexes stimulate production of type 1 interferon, a hallmark of infection that is also observed in SLE patients, regardless of disease activity. In addition to B cells and PCs, various T cell populations also exert differential effects on SLE pathogenesis. T follicular helper cell subsets contribute to B cell activation and differentiation, and abnormal T cell receptor signaling is also thought to lead to hyper-responsive autoreactive T cell activity. Furthermore, defects in regulatory T cells, partially secondary to deficient IL-2 production, result in faulty modulation of immune activity and inflammation.

Myeloid cells (MC) also play a role in SLE pathogenesis. Factors present in the local microenvironment may cause macrophages (Mϕ) to undergo extreme changes in transcriptional regulation in a process called Mϕ polarization Overabundance of proinflammatory M1 Mϕ and decreased expression of markers for anti-inflammatory M2 Mϕ are detected in both lupus-prone mice and SLE patients, and therapeutic stimulation of M2 polarization significantly decreases disease severity in murine SLE. Experimental intervention in M2 polarization as well as microRNA array profiling suggest that abnormalities in M2 Mϕ may contribute to SLE severity. Low-density granulocytes (LDGs) are abnormal neutrophil-like cells that appear in the blood of lupus patients as well as in many other disease states. Although their involvement in SLE has not been studied as extensively as that of other cell types, LDGs have already been linked to kidney disease, vascular disease, and other manifestations in lupus patients. LDG modules may be generated by WGCNA meta-analysis (manuscript in preparation), and r values indicate separation from control and SLE neutrophils.

To date, however, it has been difficult to relate gene expression profiles to SLE disease activity successfully. Many attempts have been made to characterize SLE patients by gene expression, including efforts to identify individual genes that predicted subsequent flares, and the determination of a discrete group of differentially expressed (DE) genes that may be found in subjects with SLE renal disease. extensively analyzed pediatric lupus samples and attempted to associate modules of expressed genes with disease manifestations in children. Despite these advances, none of the data has yet provided an approach with sufficient predictive value to utilize in decision making about individual subjects with SLE, nor has any cellular phenotype been independently verified to be able to distinguish a patient with active SLE from one with inactive disease. This distinction is critical both for patient evaluation and for clinical trials, as most SLE trials are aimed at controlling disease activity.

Therefore, in order to advance personalized treatment of SLE patients, the use of big data analytical techniques, including machine learning, may be useful to understand the relationships between cell subsets, gene expression, and disease activity. Machine learning describes a wide range of computational methods which allow researchers to harness complex data and develop self-trained strategies to predict the characteristics of new samples, such as whether a given SLE patient has active or inactive disease. When applied to high-throughput bioinformatics data, machine learning algorithms may identify the gene expression features with the most utility for the task at hand and may thereby provide insights into disease pathogenesis.

Conventional bioinformatics methods in conjunction with unsupervised and supervised machine learning techniques to: (1) test the potential of raw gene expression data and modules of genes to classify subjects with active and inactive SLE, (2) determine the optimum classifier or classifiers, and (3) understand the combinations of variables that best facilitate classification.

Provided herein are machine learning approaches to integrate gene expression data from multiple SLE data sets and used it to predict active disease. Both raw whole blood gene expression data and informative gene modules generated by Weighted Gene Co-expression Network Analysis from purified leukocyte populations are employed by classification algorithms. SLE whole blood gene expression data from 156 patients across three data sets are used to classify patients as having active or inactive disease as characterized by standard clinical composite outcome measures. When training and testing sets are formed by holding out entire data sets, machine learning algorithms using raw gene expression data had an average classification accuracy of only 53 percent. However, converting this gene expression data to module enrichment improved classification accuracy to 71 percent. When training and testing sets are formed by mixing patients from the three data sets, module enrichment remained at a 70 percent classification accuracy. However, classification accuracy using raw gene expression increased to a mean of 79 percent. The best overall performance came from the random forest classifier, which had a predictive accuracy of 84 percent.

Gene expression data may be compiled as follows. Publicly available gene expression data and corresponding phenotypic data may be mined from the Gene Expression Omnibus. Raw data sources for purified cell populations are as follows: GSE10325 (CD4: 8 SLE, 9 HC; CD19: 10 SLE, 8 HC; CD33: 9 SLE, 9 HC); GSE26975 (10 SLE LDG, 10 SLE Neutrophil, 9 HC Neutrophil); GSE38351 (CD14: 8 SLE, 12 HC). Raw data sources for SLE whole blood gene expression are as follows: GSE39088 (24 active, 13 inactive); GSE45291 (35 active, 257 inactive); GSE49454 (23 active, 26 inactive). 35 randomly sampled inactive patients may be taken from GSE45291 to avoid a major imbalance between active and inactive SLE patients. Active SLE may be defined as having an SLE Disease Activity Index (SLEDAI) of 6 or greater.

Quality control and normalization may be performed as follows. Statistical analysis may be conducted using R and relevant Bioconductor packages. Non-normalized arrays may be inspected for visual artifacts or poor hybridization using Affy QC plots. PCA plots may be used to inspect the raw data files for outliers. Data sets culled of outliers may be cleaned of background noise and normalized using RMA, GCRMA, or NEQC where appropriate. Data sets may be then filtered to remove probes with low intensity values and probes without gene annotation data. WB gene expression data sets may be filtered to only include genes that passed quality control in all data sets. At this juncture, differential expression (DE) analysis and Weighted Gene Co-expression Network Analysis (WGCNA) may be carried out on data sets. WB gene expression data sets may be then further processed before machine learning analysis. WB gene expression values may be centered and scaled to have zero-mean and unit-variance within each data set, and the standardized expression values from each data set may be joined for classification.

Differential expression (DE) analysis may be performed as follows. Normalized expression values may be variance corrected using local empirical Bayesian shrinkage, and DE may be assessed using the LIMMA package. Resulting p-values may be adjusted for multiple hypothesis testing using the Benjamini-Hochberg correction, which resulted in a false discovery rate (FDR). Significant genes within each study may be filtered to retain DE genes with an FDR<0.2, which may be considered statistically significant. The FDR may be selected a priori to diminish the number of genes that may be excluded as false negatives.

Weighted Gene Co-expression Network Analysis (WGCNA) may be performed as follows. Log 2-normalized microarray expression values from purified CD4, CD14, CD19, CD33, and low density granulocyte (LDG) populations may be used as input to WGCNA to conduct an unsupervised clustering analysis, resulting in co-expression “modules,” or groups of densely interconnected genes which may correspond to comparably regulated biologic pathways. For each experiment, an approximately scale-free topology matrix (TOM) may be first calculated to encode the network strength between probes. Probes may be clustered into WGCNA modules based on TOM distances. Resultant dendrograms of correlation networks may be trimmed to isolate individual modular groups of probes by partitioning around medoids and labeled using color assignments based on module size. Expression profiles of genes within modules may be summarized by a module eigengene (ME), which is analogous to the module's first principal component. MEs act as characteristic expression values for their respective modules and may be correlated with sample traits such as SLEDAI or cell type. This may be done by Pearson correlation for continuous or semi-continuous traits and by point-biserial correlation for dichotomous traits.

WGCNA modules from CD4, CD14, CD19, and CD33 cells may be tested for correlation to SLEDAI. SLEDAI information may be not available for the LDG modules, so the two modules provided are descriptive of LDGs compared to SLE neutrophils and HC neutrophils. Plasma cell modules may be generated by differential expression analysis and not WGCNA, but may be included because of the established importance of plasma cells in SLE pathogenesis.

Gene Set Variation Analysis (GSVA)-based enrichment of expression data may be performed as follows. The GSVA R package may be used as a non-parametric method for estimating the variation of pre-defined gene sets in SLE WB gene expression data sets. Standardized expression values from WB data sets may be used to test for enrichment of cell-specific WGCNA gene modules using the Single-sample Gene Set Enrichment Analysis (ssGSEA) method, which scores single samples in isolation and is thus shielded from technical variation within and among data sets. Statistical analysis of GSVA enrichment scores may be done by Spearman correlation or Welch's unequal variances t-test, where appropriate. GSVA may be performed on three SLE WB datasets using 25 WGCNA modules made from purified SLE cells with correlation or published relationship to SLEDAI, per Table 1. In the top line, orange: active patient; black: inactive patient. LDG: low-density granulocyte; PC: plasma cell.

Machine learning algorithms and parameters may be developed as follows. Three distinct machine learning algorithms may be employed to test biased and unbiased approaches to microarray data analysis. The biased approach involved GSVA enrichment of disease-associated, cell-specific modules, and the unbiased approach employed all available gene expression data in the WB. An elastic generalized linear model (GLM), k-nearest neighbors classifier (KNN), and random forest (RF) classifier may be deployed to classify active and inactive SLE patients and determine whether gene expression may serve as a general predictor of disease activity. GLM, KNN, and RF may be deployed using the glmnet, caret, and randomForest R packages, respectively.

GLM carries out logistic regression with a tunable elastic penalty term to find a balance between the L1 (lasso) and L2 (ridge) penalties and thereby facilitate variable selection. For our predictions, the elastic penalty may be set to 0.9, specifying a penalty that is 90% lasso and 10% ridge in order to generate sparse solutions. KNN classifies unknown samples based on their proximity to a set number k of known samples. K may be set to 5% of the size of the training set. If the initial value of k is even, 1 may be added in order to avoid ties. RF generates 500 decision trees which vote on the class of each sample. The Gini impurity index, a measure of misclassification error, may be used to evaluate the importance of variables. In addition to these three approaches, pooled predictions may be assigned based on the average class probabilities across the three classifiers.

Validation approaches may be performed as follows. The performance of each machine learning algorithm may be evaluated by 2 different forms of cross-validation. First, a random 10-fold cross-validation may be carried out by randomly assigning each patient to one of 10 groups. Next, as the data came from three separate studies, leave-one-study-out cross-validation may be also done to determine the effects of systematic technical differences among data sets on classification performance. For each pass of cross-validation, one fold or study may be held out as a test set, and the classifiers may be trained on the remaining data. Accuracy may be assessed as the proportion of patients correctly classified across all testing folds. Performance metrics such as sensitivity and specificity may be assessed after cross-validation by agglomerating class probabilities and assignments from each fold or study. Receiver Operating Characteristic (ROC) curves may be generated using the pROC R package.

Gene expression results may be obtained and analyzed as follows. Before employing machine learning techniques, it may be necessary to first assess whether conventional bioinformatics approaches may satisfactorily separate active SLE patient samples from those from inactive patients. DE analysis of active patient samples versus inactive patients in each whole blood study revealed major differences among data sets and considerable heterogeneity within data sets. First, the 100 most significant DE genes by FDR in each study may be used to carry out hierarchical clustering of active and inactive patient samples. Active patients separated from inactive patients in GSE45291, but separated with mixed results in GSE39088 and GSE49454.

Next, the lists of genes may be compared for commonalities. Out of 6,640 unique DE genes from the three studies, 5,170 genes are unique to one study, 1,234 are shared by two studies, and 36 are shared by all three studies, with a minimal overlap of the 100 most significant genes by FDR in each study. The only overlaps among the top 100 DE genes in each study by FDR are: TWY3 and EHBP1, shared between GSE39088 and GSE49454; and LZIC, shared between GSE39088 and GSE45291.

Furthermore, the fold change distributions of the 100 most significant DE genes in each study varied considerably. In GSE39088, 94 of the 100 most significant genes may be downregulated in active patients; in GSE45291, all of the top 100 genes may be upregulated in active patients; and in GSE49454, the top 100 genes may be more evenly distributed (41 up, 59 down). The three data sets are comprised of different patient populations and may be collected on different microarray platforms per Table 4. Still, the heterogeneity is striking. The lack of commonality among the genes most descriptive of active and inactive patients in each data set already casts doubt on whether active and inactive patients from different data sets may separate cleanly.

Patients from each study may be then joined to evaluate whether unsupervised techniques may separate active patients from inactive patients. Hierarchical clustering on the 297 unique most significant DE genes by FDR showed considerable heterogeneity, and active patients and inactive patients did not consistently separate, per the map of the top 100 DE genes by FDR from each study (combined total of 297 unique genes from the three studies) expressed in all patients. If gene expression has the potential to identify active SLE patients, conventional bioinformatics techniques failed to harness that, highlighting the need for more advanced algorithms.

Patterns of enrichment of WGCNA modules may be derived from isolated cell populations of WB that are correlated to the SLEDAI disease activity measure may be more useful than gene expression across studies to identify active versus inactive lupus patients. To characterize the relationships between SLE gene signatures from various peripheral cellular subsets and disease activity, WGCNA may be used to generate co-expression gene modules from purified populations of cells from subjects with active SLE, which may subsequently be tested for enrichment in whole blood of other SLE subjects. WGCNA analysis of leukocyte subsets resulted in several gene modules with significant Pearson correlations to SLEDAI (all |r|>0.47, p<0.05). CD4, CD14, CD19, and CD33 cells had 3, 6, 8, and 4 significant modules, respectively, per Table 1. Two low-density granulocyte (LDG) modules may be created by performing WGCNA analysis of LDGs along with either SLE neutrophils or HC neutrophils and merging the modules most strongly expressed by LDGs Two plasma cell (PC) modules may be created by using the most increased and decreased transcripts of isolated SLE plasma cells compared to SLE naïve and memory B cells.

Gene Ontology (GO) analysis of the genes within each module showed that some processes, such as those related to interferon signaling, RNA transcription, and protein translation, are shared among cell types, whereas other processes may be unique to certain cell types (Table 1) and may be used to better classify patients.

To characterize the relationships between SLE gene modules from cell subsets and disease activity in greater detail, GSVA enrichment may be performed using the 25 cell-specific gene modules in WB from 156 SLE patients (82 active, 74 inactive), per Table 4. Of the 25 cell-specific modules, 12 had enrichment scores with significant Spearman correlations to SLEDAI (p<0.05), and 14 had enrichment scores with significant differences between active and inactive patients by Welch's unequal variances t-test (p<0.05) (Table 2). Notably, each cell type produced at least one module with a significant correlation to SLEDAI in WB and at least one module with a significant difference in enrichment scores between active and inactive patients, demonstrating a relationship between disease activity in specific cellular subsets and overall disease activity in WB. However, the Spearman's rho values ranged from −0.40 to +0.36, suggesting that no one module had substantial predictive value. Furthermore, the effect sizes as measured by Cohen's d when testing active versus inactive enrichment scores ranged from −0.85 to +0.79. The CD4 Floralwhite and Orangered4 modules, which had the largest positive and negative effect sizes, respectively, showed a high degree of overlap in the enrichment scores of active and inactive patients, whereas error bars indicate mean±standard deviation. WB may be unable to fully separate active patients from inactive patients.

Analysis of individual disease activity-associated peripheral cellular subset gene modules may be not sufficient to predict disease activity in unrelated WB data sets, since no single module from any cell type may be able to separate active from inactive SLE patients. Although no single module had a sufficiently high predictive value, many cell-specific gene modules may be combined and optimized to predict disease activity in SLE patients. Moreover, the results emphasized the need for more advanced analysis to employ gene expression analysis to predict disease activity.

Machine learning results may be obtained and analyzed as follows. To assess the effectiveness of either raw gene expression or module-based enrichment techniques, SLE patients may be classified as active or inactive using two different methodologies: (1) a leave-one-study-out cross-validation approach or (2) a 10-fold cross-validation approach. GLM, KNN, and RF classifiers may be tasked with identifying active and inactive SLE patients based on WB gene expression data and module enrichment data. The performance of each classifier in each situation is shown in Table 2, and corresponding ROC curves. Area under the curve is shown in each plot. In almost all cases, the random forest classifier outperformed the GLM and KNN classifiers, although the results may be not significantly different when assessed by testing for equality of proportions (p>0.05). Pooled predictions based on the class probabilities from the three classifiers did not improve overall performance.

When cross-validating by study, the use of expression values achieved an accuracy of only 53 percent, per Table 3. This is in line with the findings that gene expression values have little to no utility when attempting to classify unfamiliar samples. When the training data and test data show little similarity to one another (e.g., they come from different data sets), the classifiers learn patterns that are unhelpful for classifying test samples. Remarkably, the use of module enrichment scores improved accuracy to approximately 70 percent.

When doing 10-fold cross-validation (Table 3), the use of raw gene expression values resulted in better performance compared to module enrichment in contrast to leave-one-study-out cross-validation. This increase in performance may be attributed to the presence of data from all three studies in both the training and test sets. In this case, the classifiers have the opportunity to learn patterns inherent to each data set, which proves useful during testing. In this circumstance, the random forest classifier may be the strongest performer with 84% accuracy (85% sensitivity, 83% specificity). The ROC curve demonstrated an excellent tradeoff between recall and fall-out.

The performance of module enrichment may be not substantially different between 10-fold cross-validation and leave-one-study-out cross-validation.

Overall, in a study-by-study approach (leave-one-study-out cross-validation), module enrichment outperformed raw gene expression. Importantly, when using the 10-fold cross-validation approach, raw gene expression outperformed module enrichment. These results indicate that disease activity classification based on raw gene expression is sensitive to technical variability, whereas classification based on module enrichment better copes with variation among data sets.

Random forest had the highest accuracy in three out of four testing scenarios. To determine whether its assessments of variable importance may be used to gain insight into directors of the identification of SLE activity, random forest classifiers may be trained on all patients from all data sets in order to identify the most important genes and modules as determined by mean decrease in the Gini impurity, a measure of misclassification error.

The most important genes and modules identified a wide array of cell types and biological functions. The most important genes encompass such diverse functions as interferon signaling, pattern recognition receptor signaling, and control of survival and proliferation. Notably, the most influential modules skewed away from B cell-derived modules and towards T cell- and myeloid cell-derived modules. As some of these modules had overlapping genes, the variable importance experiment may be repeated with modules that may be first scrubbed of any genes that appeared in more than one module before GSVA enrichment scoring. The relative variable importance scores of the de-duplicated modules correlated strongly with those of the original modules (Spearman's rho=0.73, p=5.18E-5), indicating that module behavior may be partly driven by the overlapping genes but strongly driven by unique genes. Variable importance of top 25 individual genes. LDG: low-density granulocyte; PC: plasma cell.

CD4_Floralwhite and CD14_Yellow, two interferon-related modules which maintained high importance after deduplication, may be further analyzed to study the effect of unique genes on module importance. Gene lists may be tested for statistical overrepresentation of Gene Ontology biological process terms with FDR correction on pantherdb.org. CD4_Floralwhite did not show any significant enrichment, but CD14_Yellow, which had the highest importance after deduplication, is highly enriched for genes with the “Immune Effector Process” designation (26/77 genes, FDR=9.38E-11 by Fisher's exact test). This suggests that CD14+ monocytes express unique genes that may play important roles in the initiation of SLE activity.

Several important findings on the topic of SLE gene expression heterogeneity within and across data sets have been elucidated by this study. First, DE analysis of active vs inactive patients may be insufficient for proper classification of SLE disease activity, as systematic differences between data sets may render conventional bioinformatics techniques largely non-generalizable.

Further, WGCNA modules created from the cellular components of WB and correlated to SLEDAI disease activity may improve classification of disease activity in SLE patients. The use of cell-specific gene modules based on a priori knowledge about their relevance to disease fared slightly better than raw gene expression, as it generated informative enrichment patterns, and many of the modules maintained significant correlations with SLEDAI in WB. However, these enrichment scores failed to completely separate active patients from inactive patients by hierarchical clustering.

A comparison may be then performed between the raw expression data and the WGCNA generated modules of genes in machine learning applications. Supervised classification approaches using elastic generalized linear modeling, k-nearest neighbors, and random forest classifiers may be implemented. The trends in performance when cross-validating by study or cross-validating 10-fold speak to the potential advantages and disadvantages of diagnostic tests incorporating gene expression data or module enrichment. Cross-validating by study serves as a kind of “worst-case” scenario, whereas 10-fold cross-validation serves as a “best-case.” Attempting to classify active and inactive SLE patients from different data sets and different microarray platforms during cross-validation by study may encounter challenges, but module enrichment may be able to smooth out much of the technical variation between data sets. 10-fold cross-validation simulated a more standardized diagnostic test. Although the data may be sourced from three different microarray platforms, each cohort in the test set had many similar patients in the training set to facilitate classification by gene expression. If such a test may be reliably free from technical noise, it is likely that raw gene expression may perform very well. RNA-Seq platforms, which produce transcript counts rather than probe intensity values, may display less technical variation across data sets if all samples are processed in the same way. An optimal panel of genes may be constructed that is similar to that identified by the random forest classifier, which may result in a simple, focused test to determine disease activity by gene expression data alone.

The strong performance of the random forest classifier indicates that nonlinear, decision tree-based methods of classification may be well suited to SLE diagnostics. This may be because decision trees ask questions about new samples sequentially and adaptively in contrast to other methods that approach variables from new samples all at once. Random forest is able to “understand” to an extent that different types of patients exist and that a one-size-fits-all approach may tend to misclassify those patients whose expression patterns make them a minority within their phenotype. In other words, active patients that do not resemble the majority of active patients may still have a strong chance of being properly classified by random forest.

The random forest classifier may be used to assess the importance of each gene and module in patient classification. The most important genes may be involved in a number of functions other than interferon signaling, such RNA processing, ubiquitylation, and mitochondrial processes. These pathways may play important roles in directing, or at least be indicative of, SLE disease activity. CD4 T cells originally contributed the most important modules, but when the modules may be de-duplicated, CD14 monocyte-derived modules gained importance. This suggests that unique genes expressed by CD14 monocytes in tandem with interferon genes may prove to be informative in the study of cell-specific methods of SLE pathogenesis. Furthermore, it is important to note that modules that may be negatively associated with disease activity may be just as important in classification as positively associated modules. Further study of underrepresented categories of transcripts may enhance our understanding of SLE activity.

While creating dedicated training and test sets may be preferable to cross-validation, this approach may require a large number of samples. Although there are large numbers of publicly available gene expression profiles of SLE patients, many of these profiles are not annotated with SLEDAI data. Furthermore, some data sets which include SLEDAI data show heavy class imbalance, which impedes classification. Cross-platform expression data may be integrated toward expanding the ability to classify active and inactive SLE patients.

The machine learning models developed provide the basis of personalized medicine for SLE patients. Integration of these approaches with high-throughput patient sampling technologies may unlock the potential to develop a simple blood test to predict SLE disease activity. These approaches may also be generalized to predict other SLE manifestations, such as organ involvement. A better understanding of the cellular processes that drive SLE pathogenesis may eventually lead to customized therapeutic strategies based on patients' unique patterns of cellular activation.

### Example 2: Prediction of Lupus Disease Activity by Applying a Machine Learning Approaches to SLE Gene Expression Data

The integration of gene expression data to predict systemic lupus erythematosus (SLE) disease activity may be a significant challenge because of the high degree of heterogeneity among patients and study cohorts, especially those collected on different microarray platforms. Machine learning approaches may be deployed to integrate gene expression data from three SLE data sets, and may be used to classify patients as having active or inactive disease (e.g., as characterized by standard clinical composite outcome measures). Both raw whole blood gene expression data and informative gene modules generated by Weighted Gene Co-expression Network Analysis from purified leukocyte populations were employed with various classification algorithms. Classifiers were evaluated by 10-fold cross-validation across three combined data sets or by training and testing in independent data sets, the latter of which amplified the effects of technical variation. A random forest classifier achieved a peak classification accuracy of 83 percent under 10-fold cross-validation, but its performance may be severely affected by technical variation among data sets. The use of gene modules rather than raw gene expression was more robust, achieving classification accuracies of approximately 70 percent regardless of how the training and testing sets were formed. Fine tuning the algorithms and parameter sets may generate sufficient accuracy to be informative as a standalone estimate of disease activity.

SLE is a complex, multisystem autoimmune disease that continues to be a major diagnostic as well as therapeutic challenge. There may be no definitive, specific diagnostic tools available to determine whether a patient has SLE, and diagnostic approaches in SLE have not changed in decades. Physicians still rely on clinical evaluation and a few laboratory tests, including measurement of autoantibodies and complement levels. Despite the wealth of genetic, epigenetic, and gene expression data that has emerged in the past few years at both the patient and cellular levels, none has been integrated to produce a predictive tool that may be used to evaluate an individual SLE patient.

In SLE, defects in central and peripheral tolerance allow for activation of self-reactive B cell clones and differentiation into plasmablasts/plasma cells (PCs) that secrete autoantibodies, which in turn mediate tissue damage. Genome wide association studies (GWAS) have identified numerous polymorphisms in regions encoding genes or regulatory regions that may influence B cell function, suggesting that a general state of B cell hyper-responsiveness may contribute to SLE pathogenesis. Autoantibody-containing immune complexes stimulate production of type 1 interferon, a hallmark of infection that is also observed in SLE patients, regardless of disease activity. In addition to B cells and PCs, various T cell populations also exert differential effects on SLE pathogenesis. T follicular helper cell subsets contribute to B cell activation and differentiation, and abnormal T cell receptor signaling is also thought to lead to hyper-responsive autoreactive T cell activity. Furthermore, defects in regulatory T cells, partially secondary to deficient IL-2 production, result in faulty modulation of immune activity and inflammation.

Myeloid cells (MC) also play a role in SLE pathogenesis. Factors present in the local microenvironment may cause macrophages (Mϕ) to undergo extreme changes in transcriptional regulation in a process called Mϕ polarization. Overabundance of proinflammatory M1 Mϕ and decreased expression of markers for anti-inflammatory M2 Mϕ are detected in both lupus-prone mice and SLE patients, and therapeutic stimulation of M2 polarization significantly decreases disease severity in murine SLE. Experimental intervention in M2 polarization as well as microRNA array profiling suggest that abnormalities in M2 Mϕ may contribute to SLE severity. Low-density granulocytes (LDGs) are abnormal neutrophil-like cells that appear in the blood of lupus patients as well as in many other disease states. Although their involvement in SLE has not been studied as extensively as that of other cell types, LDGs have already been linked to kidney disease, vascular disease, and other manifestations in lupus patients.

To date, however, it has been difficult to relate gene expression profiles to SLE disease activity successfully. Gene expression data analysis approaches may have challenges with producing sufficient predictive value to utilize in decision making about individual subjects with SLE. Furthermore, no cellular phenotype has been independently verified to be able to distinguish a patient with active SLE from one with inactive disease. This distinction is critical both for patient evaluation and for clinical trials, as most SLE trials are aimed at controlling disease activity.

Therefore, in order to advance personalized treatment of SLE patients, the use of big data analytical techniques, including machine learning, may be useful to understand the relationships between cell subsets, gene expression, and disease activity. Machine learning describes a wide range of computational methods to harness complex data and develop self-trained strategies to predict the characteristics of new samples, such as whether a given SLE patient has active or inactive disease. Machine learning techniques may be used, for example, to characterize lupus disease risk and identify new biomarkers based on genotypic data or urine tests. When applied to high-throughput transcriptomic data, machine learning algorithms may be used to identify the gene expression features with the most utility to identify subjects with higher degrees of disease activity and may also provide insights into disease pathogenesis.

Bioinformatics methods may be applied in conjunction with unsupervised and supervised machine learning techniques to: (1) test the potential of raw gene expression data and modules of genes to classify subjects with active and inactive SLE, (2) determine the optimum classifier or classifiers, and (3) understand the combinations of variables that best facilitate classification.

Gene expression data may be analyzed to assess SLE disease activity as follows. Before employing machine learning techniques, first an assessment was made regarding whether bioinformatics approaches may accurately separate active SLE patient samples from those obtained from inactive patients. First, three whole blood (WB) data sets (Table 5) were filtered to include only those genes which passed quality control and filtering in all three studies. Table 5 shows data sources for active (SLEDAI≥6) and inactive (SLEDAI<6) SLE WB gene expression. Data sets are listed by Gene Expression Omnibus (GEO) accession numbers. N Active/Inactive: number of active/inactive patients in data set. Range, mean, and standard deviation of SLEDAI values in each data set are provided.

TABLE 5
Accession of records by microarray platform, number of active
and inactive records, SLEDAI range, and SLEADAI mean

| | | N | N | | |
|---|---|---|---|---|---|
| | Microarray | Ac- | Inac- | SLEDAI | SLEDAI |
| Accession | Platform | tive | tive | Range | Mean (SD) |
| GSE39088 | GPL570 | 24 | 13 | 2-12 | 6.8 (2.7) |
| | (Affymetrix | | | | |
| | HG-U133+ 2.0) | | | | |
| GSE45291 | GPL13158 | 35 | 35 | 0-11 | 4.3 (3.5) |
| | (Affymetrix | | | | |
| | HG-U133+ PM) | | | | |
| GSE49454 | GPL10558 | 23 | 26 | 0-26 | 7.7 (7.2) |
| | (Illumina | | | | |
| | HumanHT-12 v4.0) | | | | |

Differential expression (DE) analysis of active versus inactive patient samples with the remaining filtered 7,848 genes revealed major differences among data sets and considerable heterogeneity within data sets. GSE39088 had only 176 DE genes with a false discovery rate (FDR) less than 0.2 and none with FDR<0.05; GSE45291 had 5850 DE genes with FDR<0.2 and 4837 with FDR<0.05; GSE49454 had 1710 DE genes with FDR<0.2 and 72 with FDR<0.05 (Data S1).

Hierarchical clustering was carried out on each study with all genes, DE genes with FDR<0.2, and DE genes with FDR<0.05 to determine whether active and inactive patients may separate into two clusters. The Adjusted Rand Index (ARI) was used to compare these clusterings to the known status of the patients. When using all genes, all three studies had ARIs near zero, indicating that clustering separated active and inactive patients no better than random chance (Table 6). Table 6 shows Adjusted Rand Index of Unsupervised Hierarchical Clustering Compared to Known Disease Activity. Data sets are listed by GEO accession numbers. GSE39088 had no genes with FDR<0.05. The “Three Consistent DE Genes” are DNAJC13, IRF4, and RPL22.

TABLE 6
Adjusted Rand Index of Unsupervised Hierarchical
Clustering Compared to Known Disease Activity

| | Adjusted Rand Index |
|---|---|
| GSE39088 | −0.04 |
| GSE39088; FDR < 0.2 | 0.19 |
| GSE39088; FDR < 0.05 | N/A |
| GSE45291 | 0.03 |
| GSE45291; FDR < 0.2 | −0.01 |
| GSE45291; FDR < 0.05 | 0.94 |
| GSE49454 | 0.04 |
| GSE49454; FDR < 0.2 | 0.14 |
| GSE49454; FDR < 0.05 | 0.14 |
| All Studies | 0.03 |
| All Studies; Three | 0.05 |
| Consistent DE Genes | |

GSE39088 and GSE49454 showed only mild improvement after filtering genes, whereas GSE45291 attained an ARI of 0.94 when using genes with FDR<0.05.

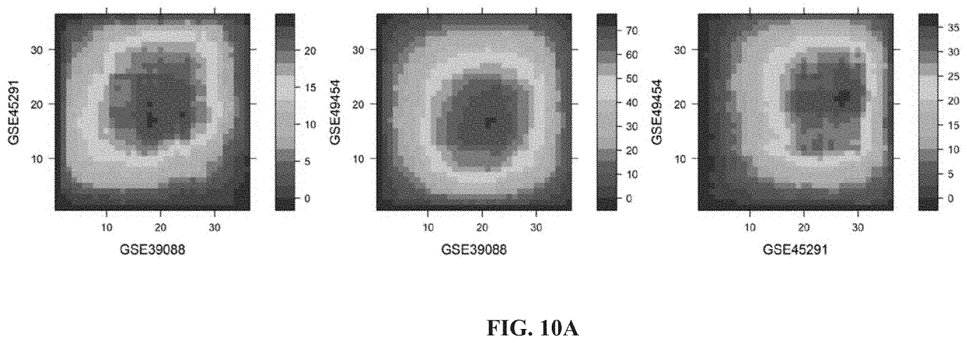

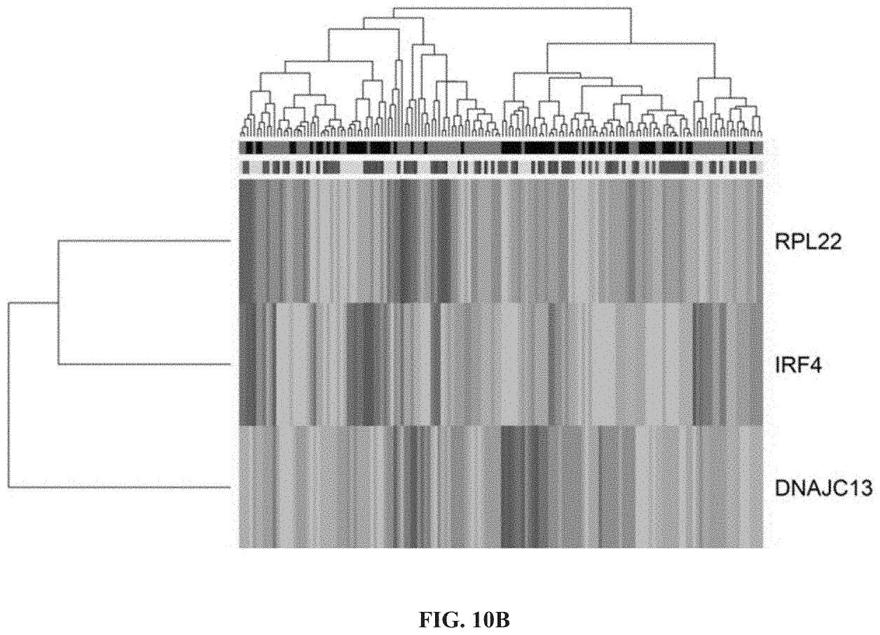

Next, the lists of genes were compared for commonalities. Out of 6,440 unique DE genes from the three studies, 5,170 genes were unique to one study, 1,234 were shared by two studies, and 36 were shared by all three studies. Of these 36 genes, only three had consistent fold changes across all studies (DNAJC13 and IRF4 upregulated; RPL22 downregulated). Rank-rank Hypergeometric Overlap (RRHO) was next applied as a threshold-free comparison of the studies (as described by, for example, Plaisier et al., “Rank-rank hypergeometric overlap: identification of statistically significant overlap between gene-expression signatures,” Nucleic Acids Res. 38, e169, which is incorporated by reference herein in its entirety). All genes that were tested for differential expression were sorted by FDR from most significantly overexpressed to most significantly underexpressed and broken into 36 groups of 218 genes each. Among the three studies, the ranked gene lists failed to demonstrate significant overlap of the most overexpressed and underexpressed genes (FIG. 10A). The three data sets were comprised of different patient populations and were collected on different microarray platforms (Table 5); still, the heterogeneity is striking. The lack of commonality among the genes most descriptive of active and inactive patients in each data set casts doubt on whether active and inactive patients from different data sets may separate cleanly.

Patients from each study were then joined to evaluate whether unsupervised techniques may separate active patients from inactive patients. Expression profiles from each study were first normalized to have zero mean and unit variance. FIG. 10B shows that even these three genes (DNAJC13, IRF4, and RPL22) failed to separate active patients from inactive patients precisely. Hierarchical clustering on all genes had an ARI of 0.03 when compared to the known status of the patients, and clustering on the three consistent DE genes shared among the studies (DNAJC13, IRF4, and RPL22) had an ARI of 0.05 (Table 6). If gene expression has the potential to identify active SLE patients robustly, bioinformatics techniques may fail to harness that potential, thereby highlighting the need for more advanced algorithms.

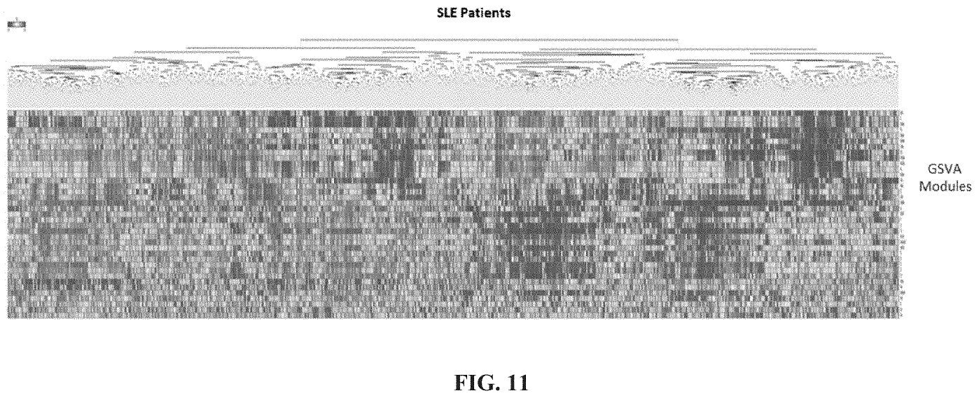

Thus far, bulk analysis of many WB and PBMC datasets on multiple platforms may show increased transcripts for IFN signature genes, granulocytes, monocytes, and plasma cells and decreased lymphocytes, but may yield little information on mechanisms of pathogenesis excepting IFN and pattern recognition receptor signaling because of the commonality of many transcripts expressed by different cell populations. Patient-specific transcriptomic “fingerprints” using readily accessible WB may be advantageously generated and analyzed to determine the relative contribution of cells, therapy, and ancestral effects, thereby providing valuable information that potentially may be used in determining entry into a clinical trial or personalized medicine strategies. FIG. 11 shows GSVA results of a lupus Illuminate gene set, demonstrating the striking heterogeneity in SLE patient WB by showing patient specific enrichment of 27 cell and process specific modules of genes. Distinct groups of lupus patients defined by GSVA groups or clusters or genes can be visually identified via the GSVA analysis. In order to understand pathogenic mechanisms of SLE, a big data analysis approach may be used on purified cell populations implicated in SLE to help understand aberrant cellular-specific mechanisms.

Patterns of enrichment of Weighted Gene Co-expression Network Analysis (WGCNA) modules derived from isolated cell populations that are correlated to the SLEDAI SLE disease activity index may be more useful than gene expression across studies to identify active versus inactive lupus patients. To characterize the relationships between SLE gene signatures from various peripheral cellular subsets and disease activity, WGCNA was used to generate co-expression gene modules from purified populations of cells from subjects with active SLE, which may subsequently be tested for enrichment in whole blood of other SLE subjects. WGCNA analysis of leukocyte subsets resulted in several gene modules with significant Pearson correlations to SLEDAI (all |r|>0.47, p<0.05). CD4, CD14, CD19, and CD33 cells yielded 3, 6, 8, and 4 modules significantly correlated to disease activity, respectively (Table 7). Table 7 shows cell module correlations to disease activity and functional analysis. Information on cell modules including number of genes, Pearson correlation coefficient to SLEDAI, and functional analysis. +: LDG modules were generated by WGCNA meta-analysis, and r values indicate separation from control and SLE neutrophils as SLEDAI was unavailable. *: PC modules are based solely on differential expression. LDG: low-density granulocyte; PC: plasma cell.

Two low-density granulocyte (LDG) modules were created by performing WGCNA analysis of LDGs along with either SLE neutrophils or HC neutrophils and merging the modules most strongly expressed by LDGs. Two plasma cell (PC) modules were created by using the most increased and decreased transcripts of isolated SLE plasma cells compared to SLE naïve and memory B cells.

TABLE 7
Cell module correlations to disease activity and functional analysis

| Cell | | Module | Correlation | | |
|---|---|---|---|---|---|
| Type | Module Name | Size | with SLEDAI | Top GO Biological Process | Top BIG-C Category |
| CD4 | Floralwhite | 237 | 0.81 | type I interferon signaling | Interferon-Stimulated- |
| | | | | pathway | Genes |
| | Turquoise | 805 | 0.50 | positive reg of ubiquitin-protein | Proteasome |
| | | | | ligase | |
| | Orangered4 | 237 | −0.77 | translational initiation | mRNA-Translation |
| CD14 | Plum1 | 247 | 0.47 | ubiquitin-dependent protein | mRNA-Translation |
| | | | | catabolic process | |
| | Yellow | 356 | 0.65 | type I interferon signaling | Interferon-Stimulated- |
| | | | | pathway | Genes |
| | Greenyellow | 89 | −0.49 | transcription from RNA | General-Transcription |
| | | | | polymerase II promoter | |
| | Pink | 261 | −0.77 | protein phosphorylation | Endosome-and-Vesicles |
| | Purple | 124 | −0.66 | inositol phosphate metabolic | Fatty-Acid-Biosynthesis |
| | | | | process | |
| | Sienna3 | 222 | −0.64 | translational initiation | mRNA-Translation |
| CD19 | Darkolivegreen | 591 | 0.78 | cell division | Proteasome |
| | Greenyellow | 251 | 0.66 | Notch signaling pathway | mRNA-Translation |
| | Steelblue | 146 | 0.65 | gluconeogenesis | Glycolysis- |
| | | | | | Gluconeogenesis |
| | Turquoise | 572 | 0.50 | ER to Golgi vesicle-mediated | Unfolded-Protein-and- |
| | | | | transport | Stress |
| | Violet | 566 | 0.61 | mitochondrial respiratory chain | Interferon-Stimulated- |
| | | | | complex I | Genes |
| | Brown | 620 | −0.62 | regulation of transcription, DNA- | Chromatin-Remodeling |
| | | | | templated | |
| | Green | 541 | −0.49 | transcription, DNA-templated | Transcription-Factors |
| | Skyblue | 756 | −0.74 | viral transcription | mRNA-Translation |
| CD33 | Royalblue | 94 | 0.60 | positive reg of cytosolic calcium | Transposon-Control |
| | | | | ions | |
| | Sienna3 | 133 | 0.76 | type I interferon signaling | Interferon-Stimulated- |
| | | | | pathway | Genes |
| | Violet | 177 | 0.79 | defense response to virus | Interferon-Stimulated- |
| | | | | | Genes |
| | Darkmagenta | 273 | −0.49 | ubiquinone biosynthetic process | MHC-Class-TWO |
| LDG⁺ | LDG_A | 334 | 0.79 | platelet degranulation | Cytoskeleton |
| | LDG_B | 92 | 0.81 | regulation of transcription | Secreted-Immune |
| | LDG_C | 82 | −0.39 | viral process | Nucleus-and-Nucleolus |
| PC* | PC_Up | 423 | N/A | protein N-linked glycosylation | Endoplasmic-Reticulum |
| | PC_Down | 183 | N/A | antigen processing and | MHC-Class-TWO |
| | | | | presentation MHC II | |

Gene Ontology (GO) analysis of the genes within each module showed that some processes, such as those related to interferon signaling, RNA transcription, and protein translation, were shared among cell types, whereas other processes were unique to certain cell types (Table 7) and may be used to classify patients more effectively. The genes in each module are listed in Table 8.

TABLE 8
Genes in modules identified via Gene Ontology (GO) analysis

| Cell | Module | |
|---|---|---|
| Type | Name | Genes |
| CD4 | Floralwhite | AARS, ABCA1, ABR, ADAM10, ADAR, AEN, AHR, AIMP1, ALOX5, ALOX5AP, APBA3, |
| | | APOL1, ARHGEF3, ARID5B, ARMCX2, ASB6, ATG4B, ATOX1, ATP1B3, ATP5J2, ATP6V1E1, |
| | | BATF, BCCIP, BCL2, C19orf66, C3orf14, CAPN2, CAPN3, CASP1, CD164, CD55, CFLAR, |
| | | CGGBP1, CHMP5, CISH, CLP1, CMTR1, CNP, CREM, CYTIP, DCAF11, DDX60, DHX58, |
| | | DNAJA1, DR1, DUSP5, EIF2AK2, EIF2S1, EIF3J, ELAC2, ENO1, ERCC1, ETV7, FAM13A, |
| | | FAM46A, FAR2, FBXL8, FCHSD2, FEM1B, GADD45B, GALNS, GCH1, GPKOW, GPR171, |
| | | GPRC5B, GSN, GTPBP1, H2BFS, HDAC9, HEMK1, HERC5, HERC6, HIST1H1C, HIST1H2BD, |
| | | HIST1H2BH, HIST1H2BK, HLA-B, HN1, ICA1, IDI1, IFI16, IFI27, IFI35, IFI44, IFI44L, IFI6, |
| | | IFIH1, IFIT1, IFIT3, IFIT5, IFITM1, IGHMBP2, IKBKE, INSL3, IPO4, IPO7, IRF4, IRF7, IRS1, |
| | | ISG15, ISG20, JUN, LAMP2, LAMP3, LAP3, LARP1, LARP7, LDHA, LGALS3BP, LGALS9, |
| | | LIMK2, LTA, LY6E, MAP4, ME3, MRPL42, MT1E, MT1F, MT1G, MT1H, MT1HL1, MT1X, |
| | | MT2A, MTM1, MTMR1, MX1, MX2, MYD88, N4BP1, NLRP2, NMI, NOP14, NPDC1, NPEPPS, |
| | | NQO2, NUP188, OAS1, OAS2, OAS3, OASL, OGFOD3, P2RX5, PARP12, PARP3, PCK2, |
| | | PDCD10, PDCD6, PDXK, PFKP, PGAM1, PGAP1, PHF11, PIGV, PIM1, PIP4K2C, PLSCR1, |
| | | PNO1, POMP, PSMA1, PSMA5, PSMB10, PSMB9, PSME1, PSME2, PTGER2, RAB11FIP1, |
| | | RASGRP3, RBCK1, RCL1, RCN1, REC8, RELB, REXO2, RMDN3, RSAD2, RTP4, RUVBL2, |
| | | SAMD9, SCO2, SELP, SEMA3G, SIPA1L1, SIRT5, SLC25A15, SNRPG, SOCS1, SOCS2, SP100, |
| | | SP110, SPATS2L, SPCS3, SQRDL, STAT1, STAT5A, STX17, SUB1, SUSD4, TAP1, TBK1, |
| | | TDRD7, TFDP2, TLR5, TLR7, TMEM140, TMSB10, TMX2, TNIP2, TRADD, TRAFD1, TRAK2, |
| | | TRANK1, TRBC1, TRIM21, TRIM22, TRIM26, TRIOBP, TSPAN13, TUBB2A, TULP4, TXNL4A, |
| | | TYMP, UBAP2, UBAP2L, UBE2L6, UCHL3, UPP1, USP11, USP18, USP46, WARS, XAF1, |
| | | YBX3, ZBP1, ZCCHC2, ZMIZ2, ZNF207, ZNF273 |
| CD4 | Turquoise | AAMDC, AASDHPPT, ABCC1, ABCC10, ACOT13, ACOT9, ACP1, ACSL1, ACTA2, ACTR3, |
| | | ACVR1, ADIPOR2, ADK, AIFM1, AIM2, AIMP2, AKAP1, ALAS1, ANAPC5, ANP32E, ANXA2, |
| | | ANXA2P2, ANXA2P3, ANXA4, APOL3, APPBP2, APTX, ARL3, ARPC1A, ARPC2, ARPC3, |
| | | ARPP19, ASCC1, ATF7IP, ATG4A, ATG5, ATIC, ATMIN, ATP2C1, ATP5G1, ATP5G3, ATP5I, |
| | | ATP5J, ATP5S, ATP5SL, ATP6V0E1, ATP6V1A, ATP6V1C1, ATP6V1D, ATP6V1H, ATPIF1, |
| | | B3GNT2, B4GALT5, BAG1, BAG5, BAK1, BAZ1A, BHLHE40, BID, BIRC3, BLVRA, BLZF1, |
| | | BORA, BTG3, BTN2A2, BUD31, BZW2, C10orf2, C11orf48, C11orf73, C14orf159, C14orf166, |
| | | C1GALT1, C1GALT1C1, C1orf50, C1QBP, C21orf59, C21orf91, C2CD3, C2orf43, C2orf44, |
| | | C2orf47, C6orf106, C8orf60, CALU, CAPZA1, CARS, CASK, CASP3, CASP4, CCDC53, |
| | | CCDC69, CCNA2, CCNB1IP1, CCNH, CCR5, CCT2, CCT3, CD28, CD38, CD59, CDC123, |
| | | CDC27, CDC73, CDK2AP1, CDK7, CDS2, CDV3, CEACAM5, CEBPG, CHCHD3, CHMP2A, |
| | | CHMP4A, CHN1, CHP1, CHST11, CHST12, CHST7, CISD1, CKS2, CLN8, CLTA, CLUAP1, |
| | | CMC2, CNDP2, CNPY2, COA3, COMMD3, COPS2, COPS5, COPS6, COQ2, COX17, COX5A, |
| | | COX5B, COX6B1, COX7A2, COX7B, CPSF6, CPT2, CRIPT, CSNK1A1, CSNK2A1, CSTF1, |
| | | CSTF2, CSTF3, CTDSP2, CTNNBL1, CTPS1, CTSK, CUL1, CUL3, CUL5, CYB5R4, CYCS, |
| | | CYLD, DBI, DCLRE1A, DCPS, DCTN5, DCTN6, DCTPP1, DDB2, DDX10, DDX19A, DDX24, |
| | | DDX27, DDX52, DDX54, DDX58, DECR1, DEF8, DERL2, DGCR2, DGUOK, DHTKD1, |
| | | DIABLO, DIMT1, DNAJC15, DNAJC2, DNAJC9, DNPEP, DNTTIP2, DOK1, DYNC1H1, |
| | | DYNC1LI1, DYNLL1, DYNLT1, EBNA1BP2, EBP, EEF1E1, EFR3A, EIF2B2, EIF2S2, EIF4A3, |
| | | EIF4E2, EIF4ENIF1, EIF5B, ELOVL6, ELP3, EMC3, EMC7, EMC8, ENDOD1, ENY2, EPS8L2, |
| | | ERAP2, ERO1L, ETF1, ETFB, ETNK1, ETS1, EZH2, EZR, F5, FABP5, FAM105A, FAM32A, |
| | | FAM50B, FAM63B, FAM69A, FANCL, FARS2, FARSA, FAS, FASTKD5, FBXO5, FBXO7, |
| | | FBXW2, FDPS, FDX1, FEN1, FH, FIBP, FIG4, FLNB, FOXK2, FRAT2, GADD45A, GALK2, |
| | | GARS, GART, GBP1, GBP2, GEMIN4, GEMIN6, GGCT, GGCX, GIGYF2, GLA, GLB1, GLG1, |
| | | GLRX2, GLRX3, GM2A, GMFG, GMNN, GMPS, GNAI3, GNPDA1, GORASP2, GOT2, GPR107, |
| | | GRAMD3, GRPEL1, GRSF1, GSTO1, GTF2A2, GTF2B, GTF2E2, GTF2H2, GTF2H5, GTPBP4, |
| | | H2AFZ, HAUS7, HCCS, HCFC2, HCP5, HDGFRP3, HDHD1, HDLBP, HEATR6, HERPUD1, |
| | | HEXIM1, HIF1AN, HIGD1A, HINFP, HIRIP3, HIST1H2AC, HMGB2, HMGCS1, HNRNPAB, |
| | | HNRNPC, HNRNPDL, HNRNPR, HPRT1, HRSP12, HSP90AA1, HSPA4, HSPA5, HSPD1, |
| | | HSPE1, HTATIP2, HTRA2, IARS, ICOS, ICT1, IDH1, IDH2, IDH3A, IDS, IER3IP1, IFT27, |
| | | IL10RB, IL13RA1, IL18R1, IL27RA, IMMT, ING2, INPP1, INPP5B, INTS12, IP6K2, IPPK, |
| | | ITFG1, ITGAE, ITGB1BP1, ITPA, JAK2, JAM2, JARID2, JMJD6, KATNA1, KCMF1, KCTD2, |
| | | KDM5A, KEAP1, KHNYN, KIAA0040, KIAA0101, KIAA0391, KIAA0586, KIAA0922, KIF22, |
| | | KLC1, KLF10, KLF12, KLHDC4, KLHL7, KPNA2, KPNA4, KPNB1, LAGE3, LAMTOR2, |
| | | LAMTOR5, LARP4, LARS2, LCMT1, LDLR, LDLRAD4, LETM1, LOC100289097, LPCAT1, |
| | | LRRC59, LRRC8D, LSM3, LXN, LYST, MAD2L1BP, MADD, MAF, MAGEF1, MANF, |
| | | MAPK13, MAPK1IP1L, MAPK9, MAPKAPK5, MBD2, MCL1, MCM3, MCM6, MCTS1, MCUR1, |
| | | MDH2, ME2, MED27, MED8, MEOX1, METTL1, METTL22, MFAP1, MFSD5, MICA, |
| | | MICALL1, MICB, MIOS, MMD, MOB1A, MPC2, MPHOSPH9, MR1, MREG, MRGBP, MRPL15, |
| | | MRPL17, MRPL20, MRPL22, MRPL3, MRPL33, MRPL46, MRPL57, MRPS11, MRPS14, |
| | | MRPS16, MRPS17, MRPS18A, MRPS18B, MRPS28, MRPS30, MRPS33, MRPS35, MTAP, |
| | | MTCH2, MTG1, MTHFD2, MTMR12, MTMR2, MTX2, MYL6, MYO5A, N4BP2L2, NAB1, |
| | | NADK, NBN, NCAPD2, NCAPH2, NCF4, NCK1, NCKAP1L, NDC80, NDUFA1, NDUFA6, |
| | | NDUFA8, NDUFA9, NDUFAB1, NDUFAF1, NDUFB3, NDUFB4, NDUFB7, NDUFB8, NDUFS2, |
| | | NDUFS3, NDUFS6, NEU1, NFE2L1, NFIL3, NFKBIE, NGFRAP1, NIPBL, NME1, NME7, NMT1, |
| | | NOD2, NOP16, NPM1, NRAS, NRBF2, NSD1, NSDHL, NSUN3, NTHL1, NUDC, NUDT21, |
| | | NUP155, NUP37, NUP93, NUP98, OBFC1, ODC1, OPTN, OSBPL3, PAF1, PAFAH1B1, PAICS, |
| | | PAK1IP1, PAK2, PAM, PANK2, PANX1, PARK7, PARN, PCIF1, PCMT1, PCTP, PDCD11, |
| | | PDCD5, PDE4B, PDE6D, PDIA6, PDPK1, PDSS1, PEX13, PEX26, PFDN2, PGD, PGM1, |
| | | PHF21A, PIGT, PIK3C3, PIK3R4, PIN4, PIP4K2A, PITPNA, PLAGL1, PLXNC1, PMAIP1, |
| | | PMS2P3, POLB, POLDIP2, POLR2I, POLR3C, POLR3K, POP4, POP7, POU2AF1, PPAP2A, |
| | | PPIE, PPM1G, PPP1R16B, PPP1R7, PPP2CA, PPRC1, PRDX3, PRDX4, PRIM1, PRKX, PRMT5, |
| | | PRPF18, PRPS2, PSEN1, PSMA2, PSMA3, PSMA4, PSMA7, PSMB1, PSMB3, PSMB7, PSMC1, |
| | | PSMC2, PSMC3IP, PSMC5, PSMD1, PSMD12, PSMD13, PSMD14, PSMD2, PSMD4, PSMD6, |
| | | PSMD9, PSMG1, PTPN2, PTRH2, PTTG1, PUS1, PWP1, QKI, QRSL1, RAB22A, RAB27A, |
| | | RAB29, RABGAP1L, RABGGTA, RABIF, RAC1, RACGAP1, RAD23B, RAD50, RAN, |
| | | RAP1GDS1, RBX1, RER1, RFC2, RFC3, RFC4, RFK, RFX5, RGS1, RHEB, RIPK1, RIT1, |
| | | RMDN1, RMND5A, RNASEH1, RNASEH2B, RNF34, RNF8, RNMTL1, RPA3, RPF1, RPL26L1, |
| | | RPL28, RPN2, RPP30, RPP40, RPS6KB1, RPUSD2, RRAS2, RRP12, RRP9, RRS1, RTCA, RTCB, |
| | | RTFDC1, RUVBL1, RWDD2B, RYBP, SAMHD1, SAP18, SAP30, SAP30BP, SAP30L, SAR1A, |
| | | SAT1, SDHA, SEC11A, SEC14L1, SEC16A, SENP5, SEPHS1, SERBP1, SERPINB1, SERPINI1, |
| | | SF3B3, SF3B5, SFPQ, SGK1, SH2D2A, SHFM1, SKAP2, SLBP, SLC16A1, SLC25A12, SLC25A4, |
| | | SLC2A3, SLC35B1, SLC35D2, SLC35F2, SLC3A2, SLC5A6, SLC7A5, SMAD3, SMAP1, |
| | | SMARCA4, SMC4, SMC6, SMCHD1, SMCO4, SMS, SNAPC3, SNF8, SNRNP25, SNRNP35, |
| | | SNRPB2, SNRPC, SNRPD1, SNRPD3, SNUPN, SNW1, SNX1, SOD1, SOS1, SP140, SP140L, |
| | | SPCS2, SPTLC2, SRD5A1, SRI, SRP19, SRSF4, STAM, STARD7, STAU1, STK17B, STK4, |
| | | STOML1, STOML2, STRAP, STX4, STX7, STX8, SUCLG1, SUMO1, SYNCRIP, SYT11, TACO1, |
| | | TAF12, TAF9, TALDO1, TARBP1, TARS, TARS2, TBC1D1, TBC1D22A, TBL2, TBXAS1, |
| | | TCEB3, TCOF1, TDP1, TESC, TFG, TFPT, TFRC, THADA, THG1L, THOC5, TIMM23, TINF2, |
| | | TIPARP, TJP2, TMCO1, TMEM11, TMEM126B, TMEM135, TMEM156, TMEM186, TMEM2, |
| | | TMEM5, TMEM62, TMEM70, TMSB4X, TNFRSF1B, TNFSF10, TNFSF8, TOM1, TOX, |
| | | TP53TG1, TPK1, TRAF3, TRAK1, TRAPPC12, TRIB1, TRIM14, TRIM38, TRIM5, TRIM68, |
| | | TSR1, TSR3, TTC1, TTC17, TUBG1, TXN, TXNL1, TXNRD1, UBAC1, UBE2A, UBE2D1, |
| | | UBE2K, UBL5, UBQLN2, UBR2, UBXN8, UCHL5, UGGT1, UMPS, UQCR10, UQCRC2, |
| | | UQCRQ, USP15, USP25, USP39, UTP11L, UTP18, UTP3, VAMP4, VAV3, VCP, VDAC1, |
| | | VDAC2, VOPP1, VRK1, VRK2, VTI1B, WBP1L, WDYHV1, WIPF2, WIPI1, WRAP53, WSB2, |
| | | WWP2, XRCC4, YARS, YEATS2, YIPF1, YLPM1, YWHAH, YWHAQ, ZBED1, ZC2HC1A, |
| | | ZDHHC4, ZMIZ1, ZNF226, ZNF536, ZNF593, ZNF710, ZPR1 |
| CD4 | Orangered4 | ABCB1, ABLIM1, ACVR1B, ADARB1, ADNP2, ALDH6A1, ALDOC, ANGEL1, ANXA1, |
| | | AP1S2, APBA2, APP, APRT, AQP3, ARCN1, ARL2BP, ARRB1, ASB8, ATXN2, ATXN7L3B, |
| | | B4GALT4, BACH2, BAG3, BNIP3L, C12orf10, C14orf1, CACNA1A, CBX7, CCDC101, CCNG1, |
| | | CCNI, CCR2, CD44, CDC37, CDIPT, CDK5R1, CERK, CHPT1, CKAP4, CMPK1, COX4I1, |
| | | COX7A2L, COX7C, CRIP1, CRK, CUTA, CUX1, DDAH2, DDOST, DIAPH1, DNAJB1, DPEP2, |
| | | DPH5, DVL1, EDEM1, EEF1D, EEF2, EIF2D, EIF3F, EIF3G, EIF3H, EIF3K, EIF3L, EIF4B, |
| | | ENO2, EP400, EPHA1, ERN2, ESD, FAM168B, FAM20B, FAM8A1, FBL, FCGRT, FGFR1, |
| | | FHL1, FOXO3, FTL, GGA1, GLO1, GLS, GPR183, GPR27, GPX4, GSS, GTF2F1, GTPBP3, |
| | | HADHA, HIP1R, HLA-F-AS1, HMCES, HNRNPA0, HOPX, HSD17B11, HSD17B8, HSF2, |
| | | HSPA1L, IGF2R, IGHD, IMPDH2, INPP5A, IRS2, ITFG2, ITPKB, KCNQ1, KLHDC2, KLRB1, |
| | | KLRG1, KPNA1, LAIR1, LAMP1, LAPTM5, LINC00623, LITAF, LSM14A, LTA4H, MAGED2, |
| | | MAN1B1, MAN1C1, MED21, METTL9, MGA, MID2, MMP24-AS1, MOB3B, NAP1L1, NCOA1, |
| | | NDRG3, NFATC2IP, NPC2, ORAI2, P4HB, PABPC1, PABPC3, PABPC4, PACSIN2, PAFAH2, |
| | | PCBP2, PDCD4-AS1, PEBP1, PFDN5, PIK3R1, PLEKHB1, PMM1, POLR1E, POU6F1, PPM1F, |
| | | PPP1R2, PPP2R5D, PRKCA, PRKD3, PRMT2, PRNP, PRUNE, PSAP, PTDSS1, PURA, QARS, |
| | | RAB11FIP3, RCC1, RCOR3, REPIN1, RGCC, RNF130, RPL11, RPL15, RPL18, RPL19, RPL22, |
| | | RPL29, RPL3, RPL35, RPL35A, RPL6, RPL8, RPLP0, RPS14, RPS16, RPS19, RPS21, RPS28, |
| | | RPS3, RPS5, RPS7, RPS9, RSL1D1, RUFY3, SCPEP1, SDHAF1, SEMA4C, SERINC5, SESN1, |
| | | SF3A3, SGSM3, SLC25A6, SLC35C2, SND1, SORL1, SPAG8, SPOCK2, SPSB3, SRSF8, SSBP2, |
| | | SSR2, SSR4, ST13, SVIL, TAF7, TBC1D5, TGFBR2, TKTL1, TMEM134, TMEM230, TOMM20, |
| | | TRAPPC6A, TRIM27, TRIM44, TRMT112, TSC22D3, TSPO, TTC9, TXN2, TXNIP, UBA52, |
| | | UBE2E3, UXT, VEGFB, VGLL4, VIPR1, VPS51, WDR41, YIPF2, ZBTB18, ZC3HAV1, ZFAND3, |
| | | ZMAT3, ZSCAN18 |
| CD14 | Plum1 | ABCD3, ADO, AKAP7, AMD1, ANKRA2, ANP32A, ANXA1, ARAP2, ARL6IP1, ARMCX1, |
| | | ARMCX3, ARPC2, ARPC3, ATP2C1, ATP6AP2, ATP6V1C1, AUH, BECN1, C1D, C5AR1, |
| | | C5orf22, C6orf62, CAPZA1, CAPZA2, CBX3, CCDC91, CCNC, CD55, CD9, CDC5L, CDC73, |
| | | CEBPB, CEBPD, CHMP2B, CHUK, CISH, CLIP1, CLPX, CNOT2, COMMD8, CPEB3, |
| | | CSGALNACT2, CTBS, CUL2, CYB5B, CYP1B1, DEK, DENR, DERA, DNTTIP2, DR1, DRAM1, |
| | | DTWD1, DUSP11, DYNLT3, E2F3, EBAG9, EDEM3, EID1, EIF3J, EIF4E, EP300, EPS15, |
| | | EWSR1, FAM216A, FOXN3, FUBP3, FUCA1, GLIPR1, GLTSCR1L, GLUL, GNPTAB, GRSF1, |
| | | HBS1L, HMGN4, HSD17B11, HUS1, IBTK, IMPACT, ISCA1, ITM2B, IVD, KCTD9, KIAA0226, |
| | | KIN, KLHL20, KTN1, KYNU, LAMP2, LAPTM4A, LARP4, LARP4B, LEPROT, LILRB2, LIN7C, |
| | | LSM5, LYN, LYPLA1, MAK16, MAP3K8, MAP4K3, MARCH7, MARS, MCM9, MEAF6, MED7, |
| | | MEF2A, MFF, MICU2, MKNK2, MTHFD2, MYO5A, NAA50, NDUFA4, NDUFA5, NDUFB1, |
| | | NFE2L2, NPTN, NUMB, NUP88, NXT2, OGFRL1, ORC4, PAIP1, PAK2, PCNP, PDHX, PDLIM5, |
| | | PDS5A, PFDN4, PICALM, PLAA, PPM1B, PPP1CB, PPP2CB, PPP2R3C, PRNP, PRRG4, |
| | | PSMD10, PSME4, PSMF1, PSPC1, PTEN, PTP4A1, QKI, RAB11FIP2, RAB27A, RAB29, RAB2A, |
| | | RAB7A, RALA, RAP2C, RBMS1, RCN2, RDH11, REST, REV3L, RFK, RMND5A, RNF103, |
| | | RNF11, RNF170, RP2, RPL37, RPL39, RTN4, SAR1B, SARAF, SAT1, SCP2, SEC23A, SEC23B, |
| | | SEMA3C, SEP15, SERBP1, SERPINB1, SHOC2, SKP1, SLC25A24, SLC35A3, SLMO2, SMA4, |
| | | SNRPA1, SNTB1, SNX10, SOCS5, SP2, SRGN, SRP9, SRSF10, ST3GAL6, STXBP3, SUB1, |
| | | SUCLA2, SUCLG2, SUMO1, SYPL1, TAF11, TBL2, TCEAL4, TCEB1, TERF1, THAP1, THOC7, |
| | | TM2D3, TMEM115, TMEM165, TMEM70, TMSB4X, TMX1, TOB1, TRAPPC13, TRIM8, |
| | | TSNAX, TSPAN31, TSPYL4, TTC37, TXNRD1, U2SURP, UBE2A, UBE2B, UBE2E1, UBE2K, |
| | | UBXN8, UFM1, UHRF1BP1L, ULK2, USP16, USP4, USP8, USP9X, UTP3, VCAN, VPS54, |
| | | WBP11, WIPI1, WWP1, XPOT, YTHDF3, YWHAB, YWHAQ, ZEB2, ZFAND6, ZFP36L1, |
| | | ZNF292, ZNF468, ZSCAN16 |
| CD14 | Yellow | ABCA1, ACSL1, ACVR1B, ADAM17, ADAP2, ADAR, ADD3, AGRN, AIM2, AIMP1, ALAS1, |
| | | ANKRD49, ARHGAP26, ARID3B, ARL4A, ATP10A, ATP11B, ATP5J, ATP6V0E1, ATP6V1E1, |
| | | ATP8B4, ATXN7, B2M, B3GNTL1, BACH1, BARD1, BCAS2, BCL10, BLVRA, BST2, BTG3, |
| | | C11orf24, C12orf5, C19orf66, C1GALT1C1, C1QA, C2orf47, C3AR1, CALM1, CALML4, CAPN2 |
| | | CASP3, CASP7, CCR1, CD2AP, CD300A, CD38, CDC40, CHIC2, CHMP5, CHPT1, CIR1, CLN5, |
| | | CMTR1, CNIH4, CNP, COA1, COX17, CREG1, CTSC, CTSL, CTSS, CUL1, CXCL10, CYLD, |
| | | DAB2, DBR1, DCTN6, DDIT4, DDX58, DDX60, DECR1, DENND1B, DHRS7B, DIAPH1, |
| | | DICER1, DNAJC15, DNASE2, DPM1, DRAP1, DYNLT1, DYSF, EIF2AK2, ENPP4, EPHB2, |
| | | EXT1, FADD, FAM175B, FAM46A, FAM65B, FAM8A1, FAS, FCGR1B, FCGR3B, FFAR2, |
| | | FKBPL, FMRI, FPR2, FYCO1, GALNT3, GBP1, GBP2, GCH1, GCLM, GCNT1, GHITM, |
| | | GLRX2, GNG5, GPN2, GPR137B, GPR65, HBP1, HEG1, HELZ, HERC5, HERC6, HIST2H2BE, |
| | | HLA-A, HLA-B, HLA-C, HLA-F, HLA-J, HNRNPA2B1, HPRT1, IFI16, IFI27, IFI35, IFI44, |
| | | IFI44L, IFI6, IFIH1, IFIT1, IFIT2, IFIT3, IFIT5, IFITM1, IFITM2, IFITM3, IFNGR1, IL15, IL1RN, |
| | | IL6ST, IQGAP2, IRF7, IRF9, ISG15, ISG20, ITFG1, JUP, KAT2B, KCNJ2, KDM5B, KDM6A, |
| | | KLF9, KLHL9, KMO, LAP3, LARP7, LGALS3BP, LIPT1, LMO2, LRRFIP1, LXN, LY6E, LY96, |
| | | MAFB, MAGOH, MAML1, MAP2K6, MARCKS, MBD2, MED28, MERTK, METTL18, METTL5, |
| | | MGAM, MILR1, MRPL16, MRPL18, MRPL19, MRPS14, MRPS22, MS4A4A, MSL2, MSMO1, |
| | | MT1E, MT1F, MT1G, MT1H, MT1HL1, MT1X, MT2A, MX1, MX2, MYC, MYD88, MYL12A, |
| | | MYL4, MYOF, N4BP1, NAB1, NAPA, NAT1, NDUFB3, NDUFS1, NECAP1, NFE2, NGRN, NMI, |
| | | NPC1, NRIP1, NT5C2, OAS1, OAS2, OAS3, OASL, PANX1, PARP12, PCMT1, PELO, PER2, |
| | | PFKP, PGK1, PHF11, PHF3, PHTF2, PIGB, PIK3CA, PIN4, PLAC8, PLAGL2, PLIN2, PLSCR1, |
| | | PML, PNO1, POLB, PPM1D, PPP2R1B, PRKAG2, PSMA4, PSMB9, PSMC2, PSMD12, PSME2, |
| | | PTPN12, PTPRO, RAB11A, RAB1A, RAB8A, RAB9A, RABGAP1L, RAPGEF2, RBM7, RBX1, |
| | | RC3H2, REC8, RGL1, RHEB, RHOA, RIN2, RNASE1, RNASE2, RNF122, RPP38, RPS27, |
| | | RPS27L, RSAD2, RTCB, RTP4, S100A11, S100A8, SAMD9, SAMSN1, SC5D, SCFD1, SEC22B, |
| | | SERPING1, SH3GLB1, SIGLEC1, SKAP2, SLA, SLC25A46, SLC30A1, SLC31A2, SLCO4C1, |
| | | SMCHD1, SNRK, SNX1, SP100, SP110, SPATS2L, SPTLC2, SQLE, SRP19, SSB, ST3GAL5, |
| | | STAT1, STAT2, STOM, STS, SWAP70, TANK, TAOK3, TAP1, TBK1, TCF4, TCF7L2, TCN2, |
| | | TDP2, TDRD7, TFEC, TFG, TFIP11, TIMP1, TLR2, TMED5, TMEM110, TMEM123, TMEM131, |
| | | TMEM255A, TMEM50A, TMPO, TNFSF10, TNS3, TOR1B, TRAF6, TRIM14, TRIM21, TRIM22, |
| | | TRIM38, TSG101, TYROBP, UBE2J1, UBE2L6, UCHL3, USP18, USP25, VAV3, VDR, VEZF1, |
| | | VRK2, VWA5A, WDFY3, WDR41, WDR5B, WDYHV1, XAF1, YME1L1, ZBTB1, ZC3HAV1, |
| | | ZCCHC2, ZNF267, ZNF322, ZNF350, ZNF443, ZNF701 |
| CD14 | Greenyellow | ACVR2A, AGTPBP1, APOD, APOL1, ARHGAP10, ASTE1, ASXL2, ATP5C1, BLM, BTBD7, |
| | | C1orf216, CAST, CCDC51, CCL5, CD27, CD3D, CEMP1, CHD4, CROT, ENSA, EP400, |
| | | EPM2AIP1, ERP44, FAM114A1, FAM208A, FBXO9, FGFR1, FLCN, FUT6, GAB1, GNA11, |
| | | HAP1, HYAL2, ITFG2, ITGAL, KANSL3, KIF21B, KLF12, KMT2A, KPNB1, KSR1, LMF1, |
| | | LOC100272216, LOC100505915, LOC647070, LPAR1, MACF1, MASP1, MICAL2, MLH3, |
| | | MMP9, MUC5AC, MYB, MYO1C, N4BP2L2, NCALD, NDST1, OCA2, PAX8, PGGT1B, |
| | | POLR1C, POLR2C, PRDM14, PRODH, RNGTT, RRP15, S1PR4, SCAF4, SEPT6, SFI1, SLC12A4, |
| | | SPN, STK39, SYT11, TBP, TCAF1, TMEM212, TMEM59L, TNNI3, TNPO3, TRAF3, TUG1, |
| | | UNC45A, USP34, VWA9, ZHX3, ZNF665, ZNF76, ZNRF4 |
| CD14 | Pink | ACAN, ACOT11, ADGRB1, AGER, AKAP8L, AKT3, ALDH2, ALDOB, ALS2CL, AMT, |
| | | ANKRD2, ARMC7, ARPP19, ATP8B2, ATXN10, BACE1, BAIAP2, BARX2, BAZ2A, BBS1, |
| | | BIN3-IT1, BNIP3L, BRAP, BRE, BTNL3, C5, C9orf9, CA1, CA14, CAD, CAMK2B, CARS, |
| | | CBX5, CBX6, CCDC71, CCDC86, CCDC9, CD1A, CDC42BPB, CDKN2A, CEACAM6, |
| | | CHRNA2, CHRNG, CISD1, CKLF, CLTA, COA7, COL1A1, COL6A2, CPD, CREBZF, CRIP1, |
| | | CSNK1G1, CTNNA1, CTSK, CYFIP2, DAXX, DGCR11, DHFR, DHX32, DNAJA3, DNPH1, |
| | | DOCK1, DPH2, DST, DYRK3, DYRK4, EIF3M, ENGASE, EPHB4, EPHB6, FAM189B, |
| | | FAM192A, FBXL5, FBXO42, FKBP4, FUT7, FXYD3, GABBR2, GAS8, GBF1, GCNT4, GDPD5, |
| | | GIPC1, GLS, GOLGA3, GPR107, GSTA1, H2AFY2, HDAC6, HDHD1, HECTD4, HFE, HMGA1, |
| | | HMGB1, HNRNPD, IKBKE, INTS5, IQCC, IQSEC2, ITPK1, JRK, KDM4C, KDM5C, KIR2DL2, |
| | | KLHDC10, LAMC1, LDB3, LDLRAD4, LGALS2, LGALS8, LINC00894, LMNA, LRCH4, |
| | | LRRN2, LUZP1, LYRM9, MAPK8IP2, MAPK8IP3, MARK4, MBP, MDK, MED12, MINK1, |
| | | MPPE1, MPPED1, MRE11A, MTOR, MUC3B, MUTYH, MYO19, MYO7A, NAA10, NACA, |
| | | NECAB3, NENF, NF2, NFATC4, NIPAL2, NKTR, NNAT, NOP14, NPEPL1, NPR2, NPTXR, |
| | | NR4A1, NSUN5P1, NTM, NUP188, OCEL1, ONECUT2, OPHN1, OPN3, PAPPA2, PCYOX1L, |
| | | PCYT2, PDCD4-AS1, PDCD6, PDGFB, PEAK1, PIGO, PIP4K2C, PIPOX, PKD2L1, PKM, |
| | | PLA2G6, PLCB3, PLCD1, PLEKHG3, POLR1D, PPIA, PPP2R4, PPP6R2, PRAF2, PRINS, |
| | | PRRC2B, PSMD4, PTCRA, PTGES, R3HDM1, RAB31, RANBP10, RAP1GAP, RAPGEF3, |
| | | RBM17, REXO2, RHO, RNASEH2B, RPGR, RPH3A, RPL35A, S100A13, SAFB2, SEC31A, |
| | | SERINC2, SF3A1, SFN, SFTPB, SHQ1, SIGMAR1, SLC15A2, SLC28A1, SLC44A1, SLC46A3, |
| | | SLC7A6, SMARCD1, SMC1A, SMPD2, SNCA, SNX11, SNX3, SORBS3, SSBP1, SSBP3, |
| | | ST6GALNAC2, STK24, SUPT20H, SUPT6H, SYT13, TARBP1, TARBP2, TBX1, TCOF1, |
| | | THUMPD2, THY1, TMEM109, TMEM147-AS1, TMPRSS15, TNK1, TNS1, TOMM34, TOP3A, |
| | | TOPORS-AS1, TPM1, TPT1, TRIT1, TRO, TTC17, TTLL12, UBAP2L, UBE3B, UBL3, UGDH, |
| | | UNC119, UNC13A, USE1, VAC14, VPRBP, VPS13D, WDTC1, WWC3, ZBTB22, ZBTB40, |
| | | ZMYM3, ZMYND11, ZNF337, ZNF592, ZNF629, ZNF839, ZSWIM8, ZZEF1 |
| CD14 | Purple | AATK, ACSL5, ADGRE3, AEBP1, AIMP2, ANXA2P1, AQP6, ARMC6, ATG4B, AVPI1, BEST1, |
| | | C14orf93, C1orf54, C22orf31, C2CD2, CASP10, CBFB, CCDC130, CDX1, CEACAM3, CKAP5, |
| | | COL8A2, CXorf56, DCUN1D4, DIMT1, DYNC1H1, EIF5B, EMID1, FAM102A, FAM206A, |
| | | FARS2, FASTK, FXYD2, GABRR2, GALT, GLP1R, GLT8D1, GPATCH8, HEATR1, HMGXB3, |
| | | HSPB6, HUWE1, IFT88, INPP5E, IPPK, ITPKC, KIAA0586, KLK3, KRT31, LAMP1, LLGL1, |
| | | LMBR1L, LRRC14, MAGT1, MAP3K10, MAP3K7, MARC2, MAST2, MECP2, MLX, MTMR9, |
| | | MYBL2, MYNN, MYO9A, NFATC1, NIT2, NSMAF, NTRK3, NUP210, OR2H2, OXSM, PBX2, |
| | | PCDH12, PCK2, PHKA2, PHLPP2, PLCG1, PLEK2, POFUT1, POU6F1, PPIG, PPP1R26, PRCP, |
| | | PRUNE, PVR, PYCR1, RAB3IL1, RAD1, RBM19, RIN1, RMDN3, RPL38, RPS11, RRAGA, |
| | | SCML2, SDHAF1, SECISBP2L, SEL1L, SLAMF8, SLC1A4, ST3GAL4, STARD8, SUPV3L1, |
| | | TBX5, TCF3, THRA, TIMELESS, TMEM2, TRIM26, TRIM45, TRIO, TRMT12, TRPM6, TUB, |
| | | UBAP2, UBE2D4, VAMP3, VPS33B, WDR70, WNT10B, ZC3H13, ZMIZ2, ZNF419, ZNF862 |
| CD14 | Sienna3 | ABCC5, ACIN1, ACP1, ACYP2, AFG3L2, AHCYL1, AHNAK, AKR7A2, ALOX5, ANAPC5, |
| | | AP2B1, APEX1, ARIH2, ARL1, ARMCX6, ASB8, ATIC, ATP5I, ATP5L, ATRN, AUP1, BTF3, |
| | | C14orf159, C2orf68, CAMLG, CAPN3, CASC3, CCDC69, CCNB1IP1, CCT3, CD244, CDC16, |
| | | CDK10, CDK19, CES2, CIITA, CKAP4, COIL, COPZ1, COX4I1, COX7C, CRTAP, CTNS, |
| | | CYP27A1, DCTD, DHX9, DUS1L, DVL1, ECHS1, EEF2, EIF1, EIF2B3, EIF2B4, EIF2B5, EIF2D, |
| | | EIF3A, EIF3D, EIF3E, EIF3F, EIF3H, EIF3K, EIF3L, EIF4B, EIF4EBP2, ENG, EPRS, FAM162A, |
| | | FAM35A, FAM49A, FBL, FBRS, FBXO21, FCER1A, FKBP11, FLU, FOLR2, FTSJ3, FUBP1, |
| | | FXN, FYN, GARS, GAS2L1, GATAD1, GLG1, GOLGB1, GOT2, GRWD1, GSS, HADHA, |
| | | HDLBP, HEBP1, HEMK1, HINT1, HLA-DMA, HLA-DQA1, HNRNPA1, HNRNPDL, IARS, ILF3, |
| | | IMPDH2, INTS3, IPO5, ISG20L2, ITPA, IVNS1ABP, KAT2A, KATNB1, KDM6B, LAS1L, |
| | | LDHB, LETMD1, LRRC47, LSG1, LSM4, LY86, LYRM4, LZTFL1, MAN1C1, MAP4, MAP4K1, |
| | | MAPK7, MBD1, MDH2, MGST2, MMS19, MPRIP, MPST, MRPS35, MXI1, NAE1, NAP1L1, |
| | | NONO, NPEPPS, NPM1, NUP93, OSBP, OXA1L, PABPC4, PAM, PCBP2, PDCD11, PFKM, |
| | | PHB2, PHF20, PMM2, PMS2P1, POLD2, POLR2H, POLR2I, PON2, PPOX, PRKCB, PRKDC, |
| | | PSKH1, PTAFR, PTCD3, QARS, RAE1, RCC1, RCN1, REPIN1, RPA1, RPL15, RPL19, RPL22, |
| | | RPL3, RPLP0, RPLP1, RPS10, RPS16, RPS17, RPS23, RPS27A, RPS3, RPS4X, RPS6, RPS7, |
| | | RPS9, RRNAD1, SDR39U1, SEC11A, SET, SFPQ, SGPL1, SGSM2, SH3YL1, SIVA1, SKP2, |
| | | SLC11A2, SLC25A5, SLC25A6, SLC5A3, SLC9A3R1, SND1, SORL1, SPCS2, SPG7, SPINT2, |
| | | SPSB3, SRPRB, SRSF4, SRSF5, ST13, STARD7, SUGP2, SYK, TAF15, TARDBP, TBC1D12, |
| | | THAP11, TPCN1, TPT1P8, TSEN34, TST, TUBG1, TXN2, UBE2I, UQCRC2, VENTX, VPS4A, |
| | | ZNF32, ZNF395 |
| CD19 | Darkolivegreen | AACS, ABCB9, ABCC4, ABCF2, ACOT7, ACOX1, ACTA2, ACTG1, ACTR1A, ADA, ADIPOR1, |
| | | AEN, AGK, AGPS, AK2, AKR1A1, ALDH18A1, ALDH3A2, ANAPC15, ANG, AP2B1, AP2S1, |
| | | APH1B, APIP, APOBEC3G, APOL1, APOO, AQP3, ARL3, ARPC2, ASF1B, ASPM, ATAD2, |
| | | ATF6, ATOX1, ATP1B3, ATP5B, ATP5C1, ATP5G1, ATP5G3, ATP5H, ATP5J, ATP5J2, |
| | | ATP8B2, AUNIP, AURKA, AURKB, B2M, B4GALT1, B9D1, BATF, BCAR3, BCCIP, BIRC5, |
| | | BLMH, BMP8B, BRAP, BSG, BUB1, BUB1B, C14orf1, C15orf39, C19orf10, C1orf216, C21orf91, |
| | | C22orf29, C2orf49, C3orf14, C6orf106, CADM1, CALML4, CALR, CALU, CAMKK2, CARHSP1, |
| | | CAV1, CCDC51, CCNA2, CCNB2, CCND2, CCNE1, CCNE2, CCR2, CCT5, CD320, CDC20, |
| | | CDC25A, CDC45, CDC6, CDCA3, CDCA4, CDCA8, CDK1, CDK2, CDK4, CDK5, CDKN2A, |
| | | CDKN2C, CDKN3, CDS2, CENPA, CENPE, CENPF, CENPM, CENPN, CEP55, CFLAR, |
| | | CHAF1A, CHCHD2, CHEK1, CHP1, CHST2, CINP, CKAP5, CLIC1, CLIC4, CLPB, CNIH1, CNP, |
| | | CNPY2, COA4, COX6A1, COX6B1, COX7A2L, COX7B, COX8A, CRADD, CREB3, CRELD2, |
| | | CSNK1E, CSNK2A1, CSRP1, CTNNAL1, CUL5, CUTA, CYC1, DARS2, DAZAP1, DCPS, DDB1, |
| | | DDX19A, DESI1, DHFR, DLGAP5, DNA2, DNAAF1, DNAJC1, DNAJC15, DNAJC3, DNMT1, |
| | | DONSON, DPP3, DTL, E2F8, EBP, EDC3, EDEM2, EEF1E1, EFCAB11, EIF2S1, EIF4A3, |
| | | EIF4G1, EIF4H, ELAVL1, ELL, EMC1, EMC6, EMC9, ERCC6L, ERGIC2, ERO1L, ESPL1, F11R, |
| | | FA2H, FADD, FANCG, FANCI, FARSA, FBXW2, FKBP1A, FKBP2, FLAD1, FOXM1, GABPA, |
| | | GABPB1, GADD45A, GADD45GIP1, GALE, GALNT14, GAR1, GARS, GART, GATB, GCLM, |
| | | GDE1, GEMIN4, GGCX, GINS1, GINS2, GINS3, GLRX, GLRX5, GMDS, GMPPA, GNAI3, |
| | | GNAS, GNB1, GORASP2, GOSR2, GOT1, GOT2, GPN2, GRB2, GTF2A2, GTF2F2, GTPBP8, |
| | | GTSE1, GUF1, H2AFV, H2AFX, HBS1L, HDLBP, HES1, HEXB, HIRIP3, HIST1H1C, HJURP, |
| | | HMBS, HMGB1, HMGB3, HMMR, HMOX2, HNRNPAB, HOXB7, HSD17B10, HSF2, HSPD1, |
| | | HYI, IARS, IDE, IDH2, IFNAR2, IGF1, IGF2BP3, IGHG1, IL12A, IL2RB, IL6ST, IMPAD1, |
| | | INPP4A, ISOC2, ITCH, ITGAX, ITGB7, KCNA3, KDM4A, KIF14, KIF15, KIF18B, KIF20A, |
| | | KIF22, KIF23, KIF2C, KIF4A, KIFC1, KLHL5, KPTN, LAMP5, LANCL2, LAP3, LARP1, LDHA, |
| | | LGALS1, LGALS3, LMNB1, LMNB2, LOC730101, LRRC42, LRRC59, LSM1, LSM12, LTB4R, |
| | | MAGEH1, MAGT1, MAPK6, MCCC2, MCFD2, MCM10, MCM4, MCM6, MDH1, MDH2, MELK, |
| | | MET, METTL1, MGAT1, MGST2, MIS18A, MKI67, MMADHC, MPDU1, MRPL12, MRPL15, |
| | | MRPL23, MRPL24, MRPL3, MRPL33, MRPL40, MRPL42, MRPL44, MRPS11, MRPS16, |
| | | MRPS17, MRPS18B, MRPS2, MRPS34, MRPS7, MRTO4, MSRB2, MTFR1, MTRR, MTX1, |
| | | MYBL2, NAA35, NAPA, NAPG, NASP, NBN, NCAPG, NCAPG2, NCAPH, NCLN, NDC1, |
| | | NDUFA1, NDUFA13, NDUFA2, NDUFA4, NDUFA6, NDUFA7, NDUFA9, NDUFAB1, |
| | | NDUFAF3, NDUFB8, NDUFS7, NEK2, NET1, NEU1, NFE2L1, NME1, NOP10, NPM1, NRBP1, |
| | | NSDHL, NTHL1, NUDT21, OGDH, OIP5, OPTN, OR7E12P, ORMDL2, OSBP, PAFAH1B3, |
| | | PAGR1, PAICS, PAK1IP1, PAK2, PARP2, PARPBP, PBK, PCCB, PDE6D, PDK1, PDXK, PGD, |
| | | PGM3, PGRMC1, PHB, PHGDH, PIM2, PKMYT1, PLA2G12A, PLAGL2, PLK4, PLOD1, PMM2, |
| | | PNO1, PNPLA4, POLA1, POLA2, POLDIP3, POLE2, POLR2D, POMP, POP7, PPA1, PPAT, |
| | | PPIA, PPIF, PPP2R1B, PPP2R2A, PPP6C, PRC1, PRCC, PRDM1, PRDX1, PRIM1, PRIM2, |
| | | PRKAG1, PRMT5, PROSER1, PRRC1, PSAT1, PSMA2, PSMA5, PSMA7, PSMB1, PSMB2, |
| | | PSMB5, PSMB6, PSMB8, PSMC1, PSMC3, PSMD11, PSMD12, PSMD14, PSMD8, PSMD9, |
| | | PSME2, PSME3, PSMG2, PTPLAD1, PXMP2, PXMP4, R3HDM1, RAB27A, RAB2A, RAB6A, |
| | | RAB8A, RABAC1, RABL6, RAD1, RAD51, RAD54B, RALA, RANBP1, RAP1A, RCC1, |
| | | RECQL4, RER1, REXO2, RFC5, RFK, RGS13, RMND5A, RNASEH1, RNASEH2A, RRAGD, |
| | | RRBP1, RRM1, RRS1, RUVBL1, S100A4, SAE1, SAMHD1, SBNO1, SCAMP2, SDF2L1, SDHB, |
| | | SEC13, SEC23IP, SEL1L, SEPHS1, SF3B5, SFXN1, SH3GLB1, SHMT1, SIL1, SKA1, SLBP, |
| | | SLC12A2, SLC16A1, SLC19A1, SLC25A11, SLC25A3, SLC25A4, SLC25A5, SLC35A2, |
| | | SLC39A14, SLC39A7, SLC7A5, SLC9A3R1, SLCO3A1, SLIRP, SMC2, SMOX, SNRPC, |
| | | SNRPD1, SNRPF, SNRPG, SP100, SPAG5, SPC25, SRM, SRPR, SRPRB, SRSF10, SSR3, |
| | | SSSCA1, STAM2, STARD7, STIL, STIP1, STRAP, SUMO3, SUPT4H1, SZRD1, TBL2, TCEB2, |
| | | TDP1, TECR, TGOLN2, THEMIS2, TIMM13, TIMM8B, TIMP2, TIPIN, TK1, TLE3, TM9SF4, |
| | | TMA16, TMED9, TMEM106C, TMEM110, TMEM147, TMEM184B, TMEM194A, TMEM248, |
| | | TMEM258, TMEM5, TMEM59, TMEM97, TMPO, TMSB10, TOP1, TOP2A, TPGS2, TPX2, |
| | | TRAPPC2L, TRAPPC3, TRIP13, TSHR, TST, TTK, TUBB2B, TUSC2, TXLNA, TXN2, TXNL4A, |
| | | UBE2C, UBE2D3, UBE2H, UBE2L3, UBE2S, UBFD1, UCHL1, UFD1L, UGGT1, UQCRC1, |
| | | UQCRFS1, UQCRQ, UROS, USP14, VAPA, VKORC1, WBSCR22, WDR1, WDR12, WDR76, |
| | | WHSC1, XRCC4, XRCC5, YARS, YIF1A, YKT6, ZDHHC3, ZNF207, ZNF35, ZNF593, ZNHIT1, |
| | | ZWILCH, ZWINT |
| CD19 | Greenyellow | ABCC1, ABHD14A, ACLY, ACO2, ACP2, ACTB, ADAP1, ADCY3, ADRA2C, AKIRIN1, |
| | | ALDH3A1, ALG3, ANO10, AP1S1, APEH, ARF3, ARFIP1, ARHGDIA, ARSA, ARTN, ATG13, |
| | | ATP13A1, ATP6V0B, AURKAIP1, BAZ1B, BOP1, BTG2, BYSL, C11orf24, C17orf53, CAD, |
| | | CCDC186, CCNF, CD99, CDK16, CHN2, CHPF, CHPF2, CLDN14, CLPP, CLSPN, CNTD2, |
| | | COMMD4, COMT, COPE, CRMP1, CSNK1D, CTPS2, CXCR3, CYP4F12, DBP, DCSTAMP, |
| | | DCTPP1, DIAPH1, DLEC1, DNAJB12, DNASE2, DOK4, DPM2, DTX3, E2F1, EHD3, EIF2AK1, |
| | | EIF3B, EIF6, ELMO1, ERLIN1, ERV9-1, EXOSC4, FAM214B, FLNB, FLNC, FN1, FOXRED2, |
| | | FTSJ2, G3BP1, GANAB, GAS6, GCDH, GGA3, GNB1L, GPR144, GPR25, GRWD1, HAPLN2, |
| | | HAX1, HDGF, HEATR2, HHLA3, HMOX1, HNRNPF, HOXC4, HSPA6, HSPBP1, IFRD2, IGH, |
| | | IGHD, IGHM, IGK, IGLL3P, IKBKE, IL13, IL1RAPL2, INTS5, IQCE, JAG1, KATNB1, KCNN3, |
| | | KCNQ4, KCTD5, KDM8, KNOP1, KPNA6, LDHC, LDLR, LEPRE1, LILRB4, LPCAT4, LRRC41, |
| | | LTK, LYPLA2, MAPKAPK3, MAST2, MBD1, MCAT, MCL1, MEF2D, MEG3, MICU1, MROH7, |
| | | MRPL34, MSMB, MSRB1, MST1L, MUC3A, NABP2, NDUFB2, NDUFB7, NEUROD4, NF2, |
| | | NFYA, NHP2, NIPAL3, NKX3-1, NOC2L, NOL3, NOLC1, NPAS1, NQO1, NUBP2, NUCB1, |
| | | OXCT2, PAFAH2, PAM16, PCDHGB6, PCYT1B, PEA15, PEPD, PEX19, PFDN1, PHTF1, PIGO, |
| | | PLA2G2D, PLIN3, PLK1, PLOD3, PNMA2, POLE, POLR2L, PPIC, PPP1R14B, PPP2R3A, |
| | | PPP5C, PRKCD, PRR5, PSEN2, PSENEN, PSMD3, PSMD5, PTBP1, PTGES2, PTP4A3, PTPN12, |
| | | PTPN18, PTPN9, PYGB, RAB5A, RAC1, RANGAP1, RASGRF1, RBM12B, REC8, RITA1, |
| | | RNF130, RRP9, RSPH6A, RXRA, SAMD14, SAP30, SARS, SARS2, SCAMP3, SEC24C, SGTA, |
| | | SIGLEC6, SIVA1, SLC12A8, SLC16A6, SLC1A5, SLC35C1, SLC4A10, SLC52A2, SLC6A2, |
| | | SPAG11A, SPN, STXBP6, STYXL1, SUMO2, SYP, TADA2A, TARBP2, TCEA1, TCF25, |
| | | TDRD12, TEX261, THAP3, THOC5, TIMM10, TKT, TMEM223, TMEM230, TOMM22, TOR3A, |
| | | TOX4, TRIP6, TSFM, UBE2N, UBE2NL, UBE2Q1, UPF1, VAC14, VARS, VAV1, VCX2, |
| | | VPREB1, WDR18, WDR62, WTAP, YIPF2, ZNF282, ZNF609 |
| CD19 | Steelblue | ABCF1, ADI1, ADSL, AHCY, ALDOA, AP3D1, ATF4, ATF5, ATP1A1, ATP2C1, B4GALT5, |
| | | BCKDK, BCL2L11, BID, C12orf43, C21orf59, C4orf27, CCDC86, CCT3, CCT7, CD58, CDK7, |
| | | CHST11, CKLF, CLP1, COG7, COPA, CYTIP, DAP, DDX39A, DENND3, DYNLRB1, ECHS1, |
| | | EDF1, EIF2B4, EIF3I, ELAC2, ENO1, ERGIC3, FAF2, FASN, FASTKD5, FTSJ1, GALNT2, |
| | | GAPDH, GCN1L1, GLO1, GPAA1, GPI, GPN1, GSS, GSTO1, GSTZ1, GTPBP4, GUK1, |
| | | HNRNPC, IMMT, IMP4, IPO4, IRAK1, KARS, LAGE3, LCMT1, LRP8, LRPAP1, LSM4, |
| | | MAD2L1BP, MAGED1, MAPKAP1, MCM5, MCM7, MCOLN1, MECR, MIF, MPHOSPH10, |
| | | MRPL11, MTMR12, NCL, NDUFS2, NDUFV2, NOL7, NQO2, NRD1, NUDC, NUDT1, NUDT15, |
| | | NUP205, NUP93, ODC1, PA2G4, PARP4, PCK2, PGAM1, PGK1, PIGT, POLD3, POLDIP2, PPIH, |
| | | PPP1R7, PSMA1, PSMB3, PSMB4, PSMC5, PSMD2, PSMD4, PUS3, RGCC, RHOB, RNF114, |
| | | RPP30, SDF4, SF3B2, SKP2, SLC2A5, SLC38A2, SLC39A8, SLC3A2, SLC43A3, SLCO4A1, |
| | | SOD1, SPTLC2, SSR2, SSRP1, ST6GALNAC4, TACO1, TBC1D15, TCEB3, TDRD7, TIMM44, |
| | | TNPO3, TRAP1, TSSC1, TTLL12, TUBA1B, TUBA1C, TUBB, TUBB3, TUBB4B, TUFM, |
| | | UBL4A, VDAC2, WARS, WDR45, XRCC6, YBX1, ZNF410 |
| CD19 | Turquoise | AARS, AASDHPPT, ACADM, ACAT1, ACSL4, ACTR2, ACVR1, ADAM10, ADSS, AKAP1, |
| | | ALG5, ALG6, AMD1, ANKRD12, ANKRD17, ANKRD36, ANP32B, ANP32E, ANXA5, ANXA7, |
| | | API5, APOBEC3B, ARF4, ARFGAP3, ARHGAP6, ARHGEF12, ARID3A, ARL1, ARL4C, |
| | | ARL5A, ARL6IP1, ARMC1, ARMCX3, ARPP19, ASNS, ATG5, ATP2A2, ATP6AP2, ATXN1, |
| | | AZIN1, B3GNT2, B4GALT3, BAG2, BARD1, BBIP1, BBS7, BCKDHB, BECN1, BIK, BRCC3, |
| | | BTN1A1, BUB3, BZW1, C1D, C1orf27, C1QBP, C2orf43, C6orf62, CAAP1, CANX, CAPN2, |
| | | CAPN7, CAPRIN1, CAPZA2, CASP10, CASP3, CBFB, CBX3, CBX6, CCNB1, CCNC, CCP110, |
| | | CD164, CD27, CD38, CD59, CD86, CDC27, CDC42, CDK14, CDK17, CDK2AP2, CDV3, CENPQ, |
| | | CENPU, CEP57, CEP97, CHST12, CHST15, CHUK, CITED2, CKAP4, CLASP2, CLCC1, |
| | | CLDND1, CLINT1, CMAHP, CNKSR1, COBLL1, COL13A1, COPB1, COPG1, CORO1C, CPOX, |
| | | CREB3L2, CRIP1, CSF2RB, CSNK1G3, CSPP1, CTBP1, CTBS, CUL2, CUL4B, CYB5B, |
| | | DAAM1, DAD1, DAPK1, DCTD, DCTN4, DCTN5, DDOST, DDX18, DDX3X, DENND1B, |
| | | DENND5B, DERL1, DERL2, DMC1, DNAAF2, DNAJA2, DNAJB9, DNAJC10, DNM1L, |
| | | DNMT3B, DSTN, DUSP5, EBAG9, ECHDC1, EDEM1, EDEM3, EED, EGLN1, EID1, EIF1AX, |
| | | EIF3A, EIF3J, EIF4E, EIF5, ELL2, ENPP3, ENTPD1, EPHA4, EPRS, ERAP1, ETFA, ETNK1, |
| | | ETS1, EXOC5, EZH2, FAIM, FAM114A1, FAM129A, FAM46C, FBXO46, FBXW7, FDX1, |
| | | FEM1B, FEM1C, FKBP11, FLI1, FNDC3A, FNDC3B, FPGT, FUBP3, FUT6, FUT8, FXR1, |
| | | G3BP2, GALK2, GALNT1, GALNT3, GBAS, GCLC, GDI2, GFPT1, GGH, GHITM, GLDC, |
| | | GLE1, GLG1, GLS, GLUD1, GLUD2, GOLPH3, GOLT1B, GPNMB, GPR15, GPRC5D, GPX7, |
| | | GSN, GSPT1, GUSBP11, H2AFY, HCFC2, HERPUD1, HIBCH, HIF1AN, HIGD1A, HIRA, |
| | | HMGB2, HMGCR, HN1, HNRNPR, HNRNPU, HRASLS2, HS2ST1, HSD17B8, HSP90B1, |
| | | HSPA13, HSPA4, HSPA5, HSPA9, HSPH1, HYOU1, IDH3A, IFT52, IGKC, IGLC1, IGLJ3, |
| | | IGLV1-44, IKZF5, IL12B, IL6R, ILF2, ILF3, IMPA1, INSIG1, IPO5, IPO7, IQCB1, IQCG, |
| | | IQGAP1, IQGAP2, IRF4, ISCA1, ISOC1, ITGA4, ITM2A, ITM2C, IVD, JUN, KCNJ13, KCTD3, |
| | | KDELR2, KDM5A, KDM6A, KIAA0101, KIF11, KLF10, KRR1, L2HGDH, LARP4, LAX1, |
| | | LIMS1, LIN7C, LINS, LITAF, LMAN1, LMAN2, LMO4, LTN1, LYPLA1, M6PR, MAD2L1, |
| | | MAN1A1, MAN1A2, MAN2A1, MANEA, MANF, MAP2K6, MAP4K3, MAPRE1, MARCH7, |
| | | MBNL2, ME2, MED13L, MED17, MFN1, MGAT2, MGLL, MLEC, MLLT10, MLX, MOB1A, |
| | | MORF4L1, MORF4L2, MRPL35, MTDH, MTF2, MTHFD2, MYO1D, MZB1, NAA50, NAB1, |
| | | NAGA, NANS, NBR1, NCOA3, NFE2L2, NFIL3, NFX1, NMD3, NNT, NONO, NRAS, NT5DC2, |
| | | NUCB2, NUDT4, NUP50, NUP98, NUS1P3, NUSAP1, NXPE3, OAT, OGT, ORC2, OSBPL3, |
| | | OSBPL9, OXR1, P4HB, PABPC4, PAPOLA, PAPSS1, PAQR3, PARM1, PDIA3, PDIA4, PDIA5, |
| | | PDIA6, PDLIM5, PEBP1, PELI1, PERP, PGPEP1, PHF7, PHYH, PIAS2, PICALM, PIGK, |
| | | PLA2G16, PLEKHA6, PLK2, POTEKP, POU2AF1, POU4F1, PPCDC, PPIB, PPP1CB, PPP1R2, |
| | | PPP3R1, PRDX3, PRDX4, PREB, PRKAG2, PRKAR1A, PRKCI, PROSC, PRPS1, PSEN1, |
| | | PSMD13, PTGES3, PTP4A1, PTP4A2, PTPN11, PTPN22, PYCR1, RAB1A, RACGAP1, RAD17, |
| | | RAD23B, RAP2B, RB1CC1, RBBP4, RBM3, RBM47, RCBTB2, RCN2, RDX, RECQL, REEP5, |
| | | RHOA, RHOQ, RIF1, RIPK1, RNF115, RNF19A, RNPEP, ROCK1, ROCK2, RPA1, RPL36AL, |
| | | RPN1, RPN2, RPRD1A, RRM2, RSRC2, RTN3, RUFY3, S100A10, SAMSN1, SCARB2, SCYL2, |
| | | SEC11A, SEC14L1, SEC22B, SEC23A, SEC24A, SEC24D, SEC31A, SEC61A1, SEC61B, |
| | | SEC61G, SEC63, SEL1L3, SELT, SEMA4A, SEPT2, SERBP1, SERP1, SGK1, SGPP1, SHCBP1, |
| | | SLAMF7, SLC1A4, SLC25A17, SLC25A46, SLC30A5, SLC33A1, SLC35A3, SLC35B1, |
| | | SLC39A6, SLC7A1, SLMO2, SMARCC1, SMC4, SMCHD1, SND1, SNX13, SNX4, SORT1, SP3, |
| | | SPAG1, SPATS2, SPCS1, SPCS2, SPCS3, SPOP, SPTLC1, SPTSSA, SRGN, SRI, SRP54, SRP72, |
| | | SRPK1, SRSF1, SRSF3, SS18, SSB, SSR1, SSR4, STEAP3, STK38L, STRN3, STT3A, SUB1, |
| | | SUCLG2, SUMO1, SUMO4, TAF2, TES, TESC, TFAM, TFB2M, TFCP2, TFDP1, TFRC, TGDS, |
| | | TLK2, TM9SF1, TM9SF2, TMBIM6, TMED10, TMED2, TMED3, TMED5, TMEM135, |
| | | TMEM165, TMEM208, TMEM39A, TMEM50B, TMEM57, TMX1, TOMM70A, TOPORS, |
| | | TOR1A, TOR1AIP1, TOX, TP53I3, TP63, TPD52, TPP2, TRA2A, TRAM1, TRAM2, TRIB1, |
| | | TRIM23, TRRAP, TSPAN31, TTC37, TUBGCP3, TWSG1, TXNDC15, TXNRD2, TYMS, |
| | | U2SURP, UAP1, UBA5, UBA6, UBE2A, UBE2E1, UBE2G1, UBE2J1, UBE3A, UBE4B, UBR5, |
| | | UBXN4, UCHL5, UFL1, UFM1, UGDH, URI1, USO1, USP46, USP8, VAMP3, VCP, VDAC1, |
| | | VDR, VIM, VOPP1, VWA9, WDR44, WDYHV1, WIPF1, WIPI1, XAF1, XBP1, XPNPEP1, XPOT, |
| | | YAF2, YIPF5, YIPF6, YTHDF2, YWHAE, YWHAH, ZBP1, ZBTB32, ZC3H13, ZDHHC13, |
| | | ZFAND1, ZFR, ZNF706 |
| CD19 | Violet | ABCE1, ACAA2, ACN9, ACOT13, ACP1, ACSL1, ADAR, AGA, AGPAT4, AIFM1, AIMP2, |
| | | ALG8, ALG9, ALKBH1, ANAPC5, ANXA2, ANXA2P1, ANXA2P2, APOL3, ARMCX5, |
| | | ARPC5L, ASAHI, ASCC3, ASUN, ATG3, ATIC, ATP13A3, ATP1B1, ATP5A1, ATP5E, ATP5L, |
| | | ATP6V1A, ATP6V1C1, AVEN, AZI2, B4GALT4, BAG1, BAK1, BLVRA, BLZF1, BORA, BPGM, |
| | | BRCA1, BTG3, BZW2, C11orf48, C11orf58, C11orf73, C12orf4, C14orf166, C14orf2, C16orf62, |
| | | C1GALT1, C2orf47, CACYBP, CAND1, CARS, CASP1, CASP6, CASP7, CBR1, CBX5, CCBL2, |
| | | CCDC53, CCDC88C, CCNH, CCR1, CCT2, CCT4, CCT6A, CCT8, CD2AP, CDC123, CDC25B, |
| | | CDC37L1, CDC5L, CDC73, CDK12, CDK2AP1, CDKN1A, CDR2, CEBPG, CEP63, CEP76, |
| | | CERS6, CETN2, CHCHD3, CHMP2A, CHMP5, CIAPIN1, CKS1B, CKS2, CLCN3, CLEC2D, |
| | | CLN5, CLTA, CMC2, CNIH4, CNOT6, COA3, COL9A3, COMMD3, COPS2, COPS3, COPS4, |
| | | COPS6, COPS8, COX17, COX5A, COX5B, COX6C, COX7A2, CPSF6, CRIPT, CSE1L, CSTF2, |
| | | CSTF3, CTPS1, CYCS, CYP11B1, DBF4, DBI, DCAF17, DCTN6, DDRGK1, DDX1, DDX10, |
| | | DDX24, DDX46, DDX49, DDX60, DERA, DHRS9, DHX15, DHX29, DIABLO, DIMT1, DLAT, |
| | | DLD, DLEU2, DNAJA1, DNAJC2, DNAJC9, DPMI, DR1, DRG1, DUT, DYNLT1, E2F3, EHD4, |
| | | EI24, EIF2AK2, EIF2B1, EIF2B2, EIF2B3, EIF2S2, EIF4E2, EIF5B, EMC3, EMC7, EMC8, |
| | | ENOPH1, ENOSF1, ENY2, ETF1, ETFDH, EXOSC2, EXOSC9, FABP5, FAHD2A, FAM206A, |
| | | FAM49A, FARS2, FASTKD2, FASTKD3, FBXO5, FECH, FEN1, FGFR1OP, FGL2, FH, FOCAD, |
| | | FOXK2, GALC, GBP1, GEMIN2, GIGYF2, GLA, GLMN, GLRX3, GLT8D1, GMNN, GNG5, |
| | | GOLGA5, GPKOW, GPR137B, GRPEL1, GRSF1, GTF2E2, GTF2H2, GTF3C3, GUSB, GYG1, |
| | | H2AFZ, HADH, HADHB, HARS, HAT1, HCCS, HDAC2, HDHD1, HEATR1, HEATR3, HEG1, |
| | | HERC5, HERC6, HIST1H2BH, HMGXB4, HNRNPD, HPRT1, HRSP12, HSD17B12, HSP90AA1, |
| | | HSPA14, HSPB11, HSPE1, HYPK, ICT1, IFI27, IFI35, IFI44, IFI44L, IFI6, IFIH1, IFIT1, IFIT3, |
| | | IFIT5, IFITM1, IFT27, INPPI, INTS12, INTS6, INTS7, ISG15, ISG20, ITFG1, ITGB1BP1, |
| | | ITGB3BP, JAK2, JMJD6, KEAP1, KIAA0020, KIAA0196, KIAA1279, KIF20B, KLC1, KLF12, |
| | | KLHL7, KPNA2, LAMP2, LAMTOR2, LCP2, LGALS8, LSM3, LSM5, MAP2K4, MAPK1IP1L, |
| | | MBIP, MCM2, MCM3, MCTS1, MCUR1, MED27, MED6, MED8, METAP2, METTL22, |
| | | METTL5, MICB, MIEF1, MLH1, MOSPD1, MPC1, MPC2, MPHOSPH9, MPP6, MRPL13, |
| | | MRPL17, MRPL18, MRPL19, MRPL20, MRPL22, MRPL46, MRPL48, MRPL57, MRPS14, |
| | | MRPS15, MRPS18A, MRPS22, MRPS27, MRPS31, MRPS33, MRPS35, MSH2, MSH6, MT1X, |
| | | MTAP, MTCH2, MTHFD1, MTX2, MX1, MX2, MYO5A, NAMPT, NARS, NARS2, NCAPD2, |
| | | NCBP1, NDC80, NDUFA8, NDUFAF1, NDUFAF4, NDUFB1, NDUFB3, NDUFB4, NDUFB5, |
| | | NDUFB6, NDUFC1, NDUFS1, NDUFS3, NDUFS4, NDUFS5, NDUFS6, NECAP1, NFYB, |
| | | NIF3L1, NINJ2, NMI, NMT1, NOD2, NPTN, NTAN1, NUP153, NUP37, NUPL1, OAS1, OAS2, |
| | | OAS3, OASL, OGFOD3, ORC5, OXCT1, PAAF1, PAIP1, PARK7, PAXIP1, PCMT1, PCNA, |
| | | PCNX, PDCD2, PDCD5, PDHA1, PDHB, PDHX, PDS5B, PDXDC1, PELO, PFDN6, PI4K2A, |
| | | PIGF, PIK3CG, PIP4K2C, PLAA, PLIN2, PLSCR1, POLE3, POLR2K, POP4, POP5, PPA2, PPID, |
| | | PPM1G, PPP1CC, PPP2CB, PPP2R5C, PPT1, PRDX6, PRPF18, PRPF4, PSMA3, PSMA4, PSMB7, |
| | | PSMB9, PSMC2, PSMC3IP, PSMD1, PSMD6, PSMD7, PSME1, PSMG1, PSPH, PSRC1, PTENP1, |
| | | PTPN2, PTRH2, PTS, PTTG1, QDPR, QKI, RAB22A, RAB40B, RAB7A, RABEPK, RAD51AP1, |
| | | RAD51C, RAE1, RAN, RANBP9, RBBP8, RBCK1, RBM15, RBMX2, RBX1, RCN1, RFC2, RFC3, |
| | | RFC4, RHEB, RIOK2, RMDN1, RMDN3, RNF103, RNF11, RPA3, RPF1, RPL26L1, RPP40, |
| | | RPS27L, RPS6KB1, RPS6KC1, RSAD2, RTF1, RTP4, RUVBL2, RWDD1, RWDD2B, SAC3D1, |
| | | SAMD9, SAR1A, SAT1, SCAMP1, SCFD1, SCO2, SCP2, SDHC, SEC23B, SHFM1, SLAMF1, |
| | | SLC20A1, SLC25A12, SLC25A20, SLC30A9, SLC35F2, SLFN12, SMAD2, SMAP1, SMARCA4, |
| | | SMARCA5, SMC3, SMCO4, SNAP29, SNF8, SNRPB2, SNRPD3, SNRPE, SOAT1, SOS1, |
| | | SPATA5L1, SPATS2L, SQLE, SQRDL, SRBD1, SRP19, SRR, SSBP1, STAG1, STAT1, STAU1, |
| | | STK17B, STMN1, STOM, STOML2, STX18, SUCLG1, SYNCRIP, SYT11, TAF1B, TAF5, TAF9, |
| | | TALDO1, TAPI, TARS, TBC1D31, TBCA, TBX21, TCEB1, TCTN3, TEX30, TFG, THG1L, |
| | | THOC7, TIMM17A, TIMM23, TIMM9, TLE4, TMCO1, TMEM126B, TMEM70, TNFSF10, TPM4, |
| | | TPRKB, TRDMT1, TRIM14, TRIM26, TSC22D1, TSG101, TSN, TTC1, TTF2, TUBG1, TWF1, |
| | | TXN, TXNL1, TXNRD1, UBAC1, UBAP2, UBE2B, UBE2K, UBE2L6, UBE2V2, UBE3C, |
| | | UBXN8, UCHL3, UFC1, UFSP2, UMPS, UQCC1, UQCR10, UQCRB, UQCRC2, USP10, USP16, |
| | | USP18, UTP11L, UTP18, VDAC3, VRK2, VTI1B, WDR61, WSB2, YIPF1, YME1L1, YWHAQ, |
| | | ZC3H15, ZDHHC4, ZFYVE21 |
| CD19 | Brown | ABCA1, ABCC5, ABCG1, ABI1, ACAP2, ACSL3, ACYP1, ADAM17, ADARB1, ADAT1, ADD3, |
| | | ADRBK2, AGL, AGPAT5, AHCYL1, AHNAK, AIDA, AIM1, AIMP1, AKAP11, AKAP9, |
| | | ALCAM, ALDH1L1, ALMS1, ALPK1, AMMECR1, ANK3, ANKRA2, ANKRD10, ANKRD10- |
| | | IT1, ANKRD36B, ANXA11, AP1AR, APAF1, APOOL, APP, APPBP2, APPL1, ARFGEF2, |
| | | ARGLU1, ARHGAP10, ARHGAP12, ARHGAP26, ARHGAP5, ARHGEF18, ARHGEF6, ARID1A, |
| | | ARID4B, ARNT, ARNTL, ARPC1B, ASAP1, ASPH, ATAD2B, ATF7IP, ATF7IP2, ATP10D, |
| | | ATP2B1, ATP8A1, ATP8B1, ATRX, ATXN10, ATXN7, AVIL, BACE2, BANK1, BAZ2B, BBS10, |
| | | BICD2, BIRC3, BLCAP, BLNK, BMP2K, BRD4, BTBD1, BTN2A2, C11orf21, C11orf80, C18orf8, |
| | | C5orf28, C9orf156, C9orf91, CA5B, CALCOCO1, CAPN3, CASP8AP2, CAT, CBFA2T3, CBR4, |
| | | CCNG2, CCNT2, CCR6, CCSER2, CD 180, CD1C, CD24, CD46, CD47, CDC40, CDC42EP3, |
| | | CDK13, CEP104, CEP135, CEP83, CHD1, CHD9, CIAO1, CIR1, CLCN4, CLEC4A, CNNM3, |
| | | CNOT8, COIL, COL5A3, CR1, CR2, CRBN, CREB1, CREBZF, CRK, CRY2, CRYL1, CSAD, |
| | | CSNK1A1, CTAGE5, CTNNB1, CTSS, CWC25, CXorf21, CYBB, CYP2E1, DAPP1, DBT, DCK, |
| | | DCLRE1C, DCP2, DCUN1D2, DCUN1D4, DDX52, DENND4A, DIAPH2, DIP2A, DIS3, |
| | | DKFZP586I1420, DLG1, DLGAP4, DNAJB14, DNAJC16, DOPEY2, DSCR3, DSE, DSERG1, |
| | | DSP, DUS2, DUSP22, DYM, DZANK1, E2F5, EAPP, EFR3A, EGR3, EIF3M, EIF4G3, ELOVL5, |
| | | ENTPD4, EPS15, ERBB2IP, ERP44, ETAA1, EVI5, EXOSC5, EXOSC7, FAM134A, FAM13B, |
| | | FAM178A, FAM179B, FAM192A, FAM49B, FAM53C, FAM63B, FAM65B, FBXO28, FBXO3, |
| | | FBXO41, FBXO42, FBXW12, FCGR2B, FCGR2C, FCRL2, FGFR1, FKBP9, FLJ42627, FMR1, |
| | | FOXN3, FRAT1, FUBP1, GALNT10, GALNT7, GATAD1, GFOD1, GLIPR1, GNE, GNG7, |
| | | GOLGA4, GPATCH8, GPR153, GPR18, GPR183, GSTA4, GTF2H3, HAUS2, HCG26, HCK, |
| | | HDAC4, HDAC9, HECTD4, HERC4, HEXA, HEXIM1, HMG20A, HMGN4, HNRNPH1, |
| | | HNRNPM, HRK, ICK, IDO1, IFNGR1, IGHV5-78, IKZF1, IL13RA1, IL15, IL6, IL7, INADL, |
| | | INPP5B, IRAK4, IRGQ, ITPR1, ITSN2, JADE3, JAG2, JRKL, KAT2B, KCNMB3, KDM3B, |
| | | KDM4C, KIAA0040, KIAA0355, KIAA0754, KIAA1033, KIAA1109, KIAA1551, KIF16B, |
| | | KLHL20, KLHL24, KMO, KMT2A, KPNA1, KPNB1, KRCC1, KRIT1, LANCL1, LAPTM4A, |
| | | LARP4B, LARS, LBH, LEMD3, LIAS, LINC00597, LOC100272216, LOC100505915, |
| | | LOC157562, LOC728093, LONRF1, LPGAT1, LPP, LRRFIP1, LRRFIP2, LSM14A, LUC7L3, |
| | | LYN, LZTFL1, MACF1, MALT1, MAP3K5, MAP3K7, MAP3K8, MAP4, MAP4K5, MARCH1, |
| | | MARCH3, MARCH6, MARCKS, MAT2B, MAVS, MBD4, MED14, MEF2A, MEF2C, METAP1, |
| | | METTL3, METTL4, METTL8, MEX3C, MFSD11, MGC12488, MGEA5, MINOS1P1, MOB3B, |
| | | MPZL1, MR1, MRPS30, MSANTD2, MSL2, MSL3, MTMR4, MVK, MYO1C, MYO1F, MZT2B, |
| | | N4BP2L1, N4BP2L2, N4BP2L2-IT2, NAA40, NAAA, NACAP1, NCOR1, NDRG2, NDUFAF7, |
| | | NEMF, NFATC2IP, NFYC, NHLRC2, NOTCH2, NOTCH2NL, NPEPPS, NR2C1, NRCAM, |
| | | NSFL1C, NUP43, OPN3, OSBPL10, OSBPL8, OSGEP, OSGEPL1, OTUD4, P2RY10, PAIP2B, |
| | | PARP12, PAXBP1, PCF11, PCMTD2, PDCD6, PDCL, PDLIM1, PDS5A, PEX12, PFKM, PGF, |
| | | PHF2, PHF20, PHKB, PHLDA1, PHTF2, PIAS1, PIKFYVE, PITPNA, PKN2, PKNOX1, |
| | | PLA2G4C, PLAG1, PLAGL1, PLEKHF2, PLEKHM1, PODNL1, POGZ, POLR1B, PPAP2A, |
| | | PPFIA1, PPIP5K1, PPP1R12A, PPP3CA, PPP6R3, PRDM10, PRDX2, PREPL, PRKAA1, |
| | | PRKACB, PRKAR2A, PRKD3, PRPF39, PRPF4B, PRR11, PRRC2C, PSME4, PSMF1, PTBP2, |
| | | PTEN, PTGER4, PTK2, PTPN6, PTPRC, PTPRK, PUM1, PYROXD1, QRSL1, RAB11FIP2, |
| | | RAB14, RAB3GAP1, RABGAP1, RABGAP1L, RAD52, RALB, RALGAPA1, RALGAPB, |
| | | RALGPS1, RALGPS2, RAP2C, RBL2, RBM25, RBM39, RBM48, RBM5, RBMS1, REL, REPS1, |
| | | REST, RFWD3, RFX5, RFX7, RGP1, RIOK3, RNF219, RNF38, RPL28, RPS15A, RPS6KA5, |
| | | RRAS2, RREB1, RSF1, RSRP1, RUFY2, RUNX1-IT1, SACS, SCAF4, SCRN1, SDCCAG3, |
| | | SEC24B, SECISBP2, SECISBP2L, SEH1L, SERGEF, SETBP1, SFTPB, SH3BP5, SHMT2, |
| | | SHOC2, SIAH1, SIRT5, SKAP2, SLC15A2, SLC25A24, SLC2A3, SLC2A6, SLC35D2, SLC35E1, |
| | | SLC35E3, SLC38A6, SLC46A3, SLC4A7, SLK, SMA4, SMARCA2, SMC6, SMYD2, SNAP23, |
| | | SNAPC3, SND1-IT1, SNX10, SNX3, SPAG16, SPG11, SPG21, SPIDR, SPTBN1, SRPK2, SRSF11, |
| | | ST13, ST8SIA4, STAP1, STEAP1, STK38, STRN, STX7, SUN1, SUPT20H, SUV420H1, SWAP70, |
| | | SYK, SYNRG, TAB2, TAF9B, TANK, TAOK1, TARBP1, TASP1, TBC1D5, TBC1D9, TCF12, |
| | | TCF4, TCL1B, THAP9-AS1, THOC1, TIA1, TIPRL, TLK1, TM2D1, TMBIM4, TMCC2, |
| | | TMEM168, TMEM212, TMEM41B, TMEM63A, TMEM9B, TNFAIP8, TNFRSF10C, TNKS2, |
| | | TOB2, TPR, TRAPPC2, TRIB2, TRIM38, TRIM52, TRIO, TRMT13, TSC22D2, TSNAX, |
| | | TSPAN13, TSPAN3, TSPYL1, TSPYL4, TSPYL5, TSR1, TTC13, TTN, TTR, UBE2D4, UBE3B, |
| | | UBP1, UBQLN4, UBXN7, USP15, USP22, USP33, USP34, USP4, USP47, USP6, USP6NL, |
| | | USP9X, UST, UTP6, UTRN, UVRAG, VAV3, VPS13A, VPS13B, VPS13C, WAPAL, WDR11, |
| | | WDR60, WDR77, WDR82, WNK1, WWC3, WWOX, XIST, YTHDC2, YWHAB, ZBED2, ZBTB1, |
| | | ZBTB20, ZBTB24, ZC3H7B, ZCCHC11, ZFC3H1, ZFX, ZKSCAN7, ZMYM6, ZMYND11, |
| | | ZNF107, ZNF142, ZNF146, ZNF154, ZNF160, ZNF26, ZNF273, ZNF280D, ZNF33B, ZNF354A, |
| | | ZNF43, ZNF468, ZNF506, ZNF510, ZNF518A, ZNF529, ZNF532, ZNF562, ZNF573, ZNF587, |
| | | ZNF611, ZNF665, ZNF669, ZNF675, ZNF701, ZNF721, ZNF75D, ZNF764, ZNF85, ZNHIT6 |
| CD19 | Green | ABCB7, ABHD10, ABHD6, ACAA1, ACO1, ACTR3B, ACYP2, ADD2, ADIPOR2, ADK, ADO, |
| | | ADPGK, ADPRM, ADRB2, AGO1, AGO4, AKAP10, AMIGO2, ANGEL2, ANKH, ANKRD26, |
| | | ANKRD27, ANKRD6, AP4S1, APC, AQR, ARFGEF1, ARHGAP19, ARHGAP24, ARHGAP32, |
| | | ARHGEF10, ARHGEF5, ARHGEF9, ARIH1, ARL8B, ARMCX2, ATG12, ATG7, ATP5S, |
| | | ATP6V0E1, ATP6V1H, ATP7A, ATP9B, ATRN, AVL9, BAG5, BBS4, BBS9, BDH2, BPHL, BRE, |
| | | BRWD1, BTBD3, BTBD7, C10orf2, C11orf30, C11orf95, C1orf109, C2CD2, C2orf42, C2orf44, |
| | | C9orf78, CACFD1, CALCOCO2, CAMSAP1, CARKD, CARS2, CASP4, CBX1, CCDC25, |
| | | CCDC28A, CDC16, CDYL, CELSR1, CENPI, CEP162, CEPT1, CFAP44, CFDP1, CGRRF1, |
| | | CHD1L, CLNS1A, CNTNAP2, COA1, COQ7, COX11, COX7C, CPQ, CRCP, CREBL2, CRKL, |
| | | CROT, CRYBG3, CRYZL1, CTNS, CTR9, CTSK, CUL3, CUL4A, CUZD1, CXorf57, CYP2C8, |
| | | DAPK2, DAZAP2, DAZL, DCLK2, DDX27, DDX28, DDX42, DEGS1, DENND2D, DFNA5, |
| | | DHX40, DHX57, DIEXF, DLG3, DNAJA3, DNAJC8, DNASE1L1, DOPEY1, DPF2, DPH5, DPP8, |
| | | DST, DUS4L, DYNC1H1, EBLN2, EIF2B5, ENAH, ENTPD1-AS1, EPM2A, EPM2AIP1, ERCC3, |
| | | ERCC5, EXOC1, EXOC2, EXT2, FAM149B1, FAM172A, FAM50A, FAM50B, FAN1, FANCF, |
| | | FBXL14, FBXL4, FEZ2, FGGY, FHL1, FIG4, FKTN, FMO5, FNBP1L, FNTA, FOXJ3, FRAT2, |
| | | FTH1, FTSJ3, GALNT11, GAPVD1, GAS2, GCC1, GCNT1, GGNBP2, GIN1, GLTSCR1L, |
| | | GNG11, GNL3L, GNPAT, GOLGA1, GPATCH1, GPM6A, GPM6B, GPR65, GSTA1, HARS2, |
| | | HAUS5, HCP5, HDDC2, HEATR6, HEMK1, HILPDA, HLCS, HMGCS1, HN1L, HNRNPH3, |
| | | HOMER1, HPS1, HPS4, HS3ST1, HSD17B7, HSDL2, IDI1, IFFO1, IFNAR1, IFT74, IL18, IL24, |
| | | IL27RA, IMPA2, ING1, INTS9, INVS, IP6K2, IRAK3, ITGAE, ITGB5, ITPR2, IVNS1ABP, JAM3, |
| | | KANSL2, KATNA1, KCTD2, KIAA0586, KIAA0753, KIDINS220, KIZ, KLF3-AS1, KLHDC10, |
| | | KRT18, KYNU, L3MBTL1, LAMP1, LARGE, LARS2, LASPI, LCMT2, LEP, LETM1, LGR4, |
| | | LINC00667, LMO2, LOC100129361, LOC389906, LPCAT3, LRRC1, LRRC47, LRRC8B, LSG1, |
| | | LUC7L, LYRM1, MAEA, MAGEF1, MAMLD1, MAP2K1, MAP2K7, MAP3K7CL, MAP3K9, |
| | | MAPK14, MARS, MAT2A, MCCC1, MCF2L, MDC1, METTL13, MICALL1, MID1, MIPEP, |
| | | MKRN2, MLH3, MORC4, MPPE1, MPPED2, MRFAP1L1, MRS2, MTERF1, MTMR2, MTMR3, |
| | | MTOR, MTRF1, MTUS1, MYBL1, MYH3, MYO1B, MYO1E, MYOM1, NACA, NAIP, NBEA, |
| | | NCBP2, NCKAP1L, NFS1, NHP2L1, NIPBL, NKRF, NOP14-AS 1, NPAT, NPC1, NPFF, NSMAF, |
| | | NSMCE4A, NUBP1, NUDCD3, NUDT13, NUP160, OARD1, OCRL, OPA1, OR10H1, OSBPL2, |
| | | OVGP1, PAPD7, PCM1, PCYT1A, PDCD11, PDZD8, PEX3, PEX5, PFAS, PHACTR1, PHF3, |
| | | PHIP, PIAS3, PIAS4, PIGB, PIGV, PJA1, PLCXD1, PLEKHA8P1, POLI, POLR1C, POLR2J4, |
| | | PON2, POU2F1, PPARD, PPARGC1A, PPCS, PPFIBP2, PPM1B, PPP1R12B, PRPF6, PRPSAP1, |
| | | PRR5L, PSPC1, PTCD3, PTPRN2, PUM2, PUS1, QTRTD1, RAB9A, RABEP1, RBM41, REPS2, |
| | | REV1, RFPL3S, RGS7, RHOH, RMND5B, RNASEH2B, RNFT2, RNMT, RPA4, RPL10L, |
| | | RPL23AP32, RPL37, RPL37A, RPP38, RPS6KA2, RRAGB, RRN3P1, RRP12, RSL1D1, RTN1, |
| | | RUFY1, RWDD3, SAMM50, SAYSD1, SCAF8, SCAPER, SCD, SCN2B, SCRIB, SDHAF1, |
| | | SEC14L1P1, SEC16A, SEC22A, SEC62, SEMA3F, SEPHS2, SEPT7, SERPINB6, SETD4, SETX, |
| | | SF3B3, SIK2, SLA, SLC24A1, SLC25A13, SLC25A37, SLC25A38, SLC36A1, SMARCAL1, |
| | | SMEK2, SMIM14, SNAPC4, SNRNP200, SNRNP35, SNX5, SOBP, SON, SP140, SPATA2, |
| | | SPRY1, SRD5A1, SS18L1, ST3GAL6, ST6GAL1, STK17A, STRADA, STX12, STX2, SUPT16H, |
| | | SUPT7L, SYF2, SYT17, TACC1, TBCC, TBL1X, TBRG4, TBX19, TBXA2R, TCEAL1, TCEAL4, |
| | | TCF7L2, TCL6, TDRD3, TECPR2, THNSL2, THOC2, TIMM22, TIMM8A, TJAP1, TLDC1, TLE1, |
| | | TLR1, TM6SF1, TMA7, TMEM127, TMEM186, TMEM231, TMEM251, TMEM62, TNFSF4, |
| | | TOMM20, TOMM34, TOP2B, TOPORS-AS1, TP53TG1, TPH1, TPST1, TPT1P8, TRAF3IP3, |
| | | TRAK2, TREML2, TRIM32, TSEN2, TTC19, TTLL5, TUBBP5, UBAP2L, UBE2D2, UBE4A, |
| | | UCN, UGT2B28, UIMC1, UNC119B, UPF3A, URB2, URGCP, UROD, USPL1, VCL, VIPAS39, |
| | | VPRBP, VPS26A, VPS33B, VPS37C, VPS41, WBP1L, WDR48, WDR73, WRAP73, WWP2, XPA, |
| | | XYLT2, YARS2, YLPM1, YPEL1, YWHAZ, ZBTB3, ZCCHC24, ZCWPW1, ZFYVE26, ZHX3, |
| | | ZKSCAN4, ZMYM1, ZMYND8, ZNF10, ZNF112, ZNF133, ZNF135, ZNF140, ZNF16, ZNF165, |
| | | ZNF180, ZNF189, ZNF200, ZNF202, ZNF213-AS1, ZNF223, ZNF224, ZNF225, ZNF227, ZNF23, |
| | | ZNF236, ZNF239, ZNF254, ZNF271, ZNF337, ZNF350, ZNF394, ZNF415, ZNF432, ZNF45, |
| | | ZNF473, ZNF493, ZNF516, ZNF544, ZNF571, ZNF587B, ZNF614, ZNF623, ZNF638, ZNF671, |
| | | ZNF696, ZNF7, ZNF710, ZNF74, ZNF813, ZNF93, ZSCAN26, ZSCAN9 |
| CD19 | Skyblue | ABAT, ABCA11P, ABCB4, ABCD4, ABLIM1, ACACB, ACSL5, ACTR5, ADAM28, ADAP2, |
| | | ADCK2, ADCK3, ADCY7, ADD1, ADNP2, AEBP1, AGBL2, AHCYL2, AKAP8, AKR7A2, |
| | | AKT3, ALAD, ALDH2, ALG13, ALOX5, AMPD3, AMT, ANKEF1, ANKMY1, ANKRD11, |
| | | ANKRD49, ANKZF1, AP1S2, AP2A2, APLP2, APPL2, ARAP2, ARHGAP15, ARHGAP17, |
| | | ARHGAP25, ARHGEF7, ARID5B, ARIH2, ARL4A, ARL6IP5, ASB1, ASB13, ASMTL, ASTE1, |
| | | ASXL1, ATG14, ATG4B, ATM, ATXN7L3B, AUTS2, B3GALT4, B3GNTL1, BACH2, BANP, |
| | | BCL2, BCL6, BCLAF1, BCORL1, BEND5, BEX4, BINI, BNIP3L, BPTF, BRD1, BRD3, BTAF1, |
| | | BTG1, BTN2A1, C10orf76, C12orf29, C14orf93, C21orf33, C2orf68, C3orf18, C5orf45, C6orf120, |
| | | CAMLG, CAMTA2, CAPRIN2, CASD1, CAST, CBFA2T2, CBLB, CBLL1, CBR3, CBX7, |
| | | CCBL1, CCDC101, CCDC109B, CCDC22, CCDC93, CCNB1IP1, CCNG1, CCNI, CCNL1, |
| | | CCNL2, CCR7, CD1A, CD1D, CD200, CD22, CD244, CD2BP2, CD40, CD55, CD69, CD96, |
| | | CDC14A, CDK10, CDK19, CDK5RAP1, CDK5RAP3, CDKN1C, CECR7, CELF1, CEP164, |
| | | CEP170, CEP68, CHCHD7, CHD7, CHI3L2, CHMP1B, CHTOP, CLASRP, CLCN6, CLEC11A, |
| | | CLK1, CLK4, CLMN, CMPK1, CNBP, CNOT2, CNPPD1, CNTRL, COL4A3, CREBBP, CRELD1, |
| | | CRLF3, CSDE1, CSRNP2, CTDSP2, CTNNBL1, CTSB, CUX1, CXCR4, CYFIP2, CYHR1, CYLD, |
| | | DAG1, DCAF10, DCAF8, DDHD2, DDX17, DEK, DENND4C, DEPDC5, DFFB, DGKD, |
| | | DHRS12, DICER1, DIDO1, DIP2C, DMTF1, DMXL1, DNAJC11, DOK1, DPEP2, DPYD, DSTYK, |
| | | DVL1, DYNLT3, DYRK1A, DYRK2, ECD, ECHDC2, EEF1A1, EEF1D, EFCAB14, EGR1, |
| | | EIF1B, EIF3E, EIF3F, EIF3L, EIF4B, ELL3, ENGASE, EP400, EPB41L2, ESD, EVL, EXOC3, |
| | | EXOSC10, EZH1, FAM111A, FAM134C, FAM160B2, FAM168A, FAM168B, FAM193A, |
| | | FAM208A, FAM32A, FAM46A, FAM60A, FBXL12, FBXL15, FBXL5, FBXO11, FBXO21, |
| | | FBXO9, FKBP15, FLJ10038, FNBP1, FNBP4, FOXJ2, FOXO1, FRYL, FUCA1, FYCO1, |
| | | GABBR1, GCC2, GDPD3, GGA2, GGPS1, GIT2, GLOD4, GMEB2, GMFB, GNA11, GNA12, |
| | | GNB5, GOLGA7, GOLGA8A, GON4L, GOSR1, GPBP1L1, GPR107, GPRASP1, GRAMD1B, |
| | | GSAP, GSDMB, GSE1, GSTM4, GTF3C2, GVINP1, H2AFJ, HAGH, HBP1, HEBP1, HECA, |
| | | HERC1, HFE, HIVEP2, HLA-DQB1, HLA-E, HLA-F, HLA-F-AS1, HNRNPA0, HNRNPA1, |
| | | HNRNPA3, HNRNPDL, HNRNPL, HPS6, HSBP1, HSD17B11, HSPBAP1, HTATIP2, HTRA2, |
| | | HUWE1, ICAM3, ID3, IER5, IFT57, IFT88, IKBKB, IL11RA, IL16, IL4R, ING4, INPP5D, IRS2, |
| | | IST1, ITM2B, JADE1, JADE2, JAK1, JARID2, JMJD1C, JRK, KAT2A, KAT6A, KBTBD2, |
| | | KCNQ1, KDM2A, KDM3A, KDM4B, KDM5B, KDM7A, KIAA0141, KIAA0226L, KIAA0247, |
| | | KIAA0430, KIAA0907, KIAA0922, KIAA0930, KIAA1467, KLF11, KLHDC2, KLHL22, KPNA4, |
| | | KRBOX4, LAIR1, LAMC1, LDOC1, LETMD1, LHFPL2, LIN37, LINC00094, LINC00341, LIPT1, |
| | | LMAN2L, LMBR1L, LMF1, LOC202181, LOC647070, LOC728392, LPIN2, LRIG1, LRRC37A2, |
| | | LRRC40, LTA4H, LTB, LY75, LYRM9, LYST, MADD, MAML1, MAN1B1, MAN1C1, |
| | | MAN2A2, MAN2B2, MANBA, MAP2K5, MAP3K4, MAP4K4, MAPK1, MAPK9, MAPKAPK5- |
| | | AS1, MAPRE2, MARCH8, MARCKSL1, MAX, MBNL1, MCM3AP, MEAF6, MECP2, MED13, |
| | | MED23, METRN, METTL17, MGA, MGAT5, MIA3, MICA, MICAL3, MKKS, MKNK1, MKRN1, |
| | | MLYCD, MOAP1, MPRIP, MTCH1, MTERF4, MTF1, MTMR1, MTMR9, MYCBP2, MYL12B, |
| | | MYO9A, MZF1, NADSYN1, NAP1L1, NAT10, NBPF1, NCK2, NCR3, NDRG3, NDST2, |
| | | NECAP2, NEK7, NEK9, NFATC1, NFATC3, NGRN, NISCH, NKTR, NLRP1, NOD1, NR3C1, |
| | | NR3C2, NREP, NRF1, NRIP1, NSUN5P1, NT5E, NUP210, NUP214, NUP88, OAZ2, OFD1, |
| | | OGG1, OSER1, OTUD3, P2RX5, P2RY14, PAFAH1B1, PAN2, PANK4, PAPOLG, PARP6, |
| | | PARP8, PASK, PATZ1, PCBP2, PCGF3, PCNT, PCNXL2, PDE8A, PECAM1, PEG10, PFDN5, |
| | | PGAP3, PGS1, PHC1, PHF11, PHF21A, PHKA2, PHLPP2, PIBF1, PIGA, PIGG, PIK3C2B, |
| | | PIK3CD, PIK3R1, PIK3R4, PILRB, PIN4, PISD, PKI55, PKIA, PLCL2, PLEKHJ1, PNISR, PNN, |
| | | PNRC1, POLG2, POLR2G, PPM1F, PPOX, PPP1R16B, PPP6R2, PRDM2, PRDM4, PRKCB, |
| | | PRKCZ, PRKRIR, PRKX, PRMT2, PRNP, PRPF3, PRRC2B, PRUNE, PSIP1, PTPLB, PTPRE, |
| | | PTPRO, PTTG1IP, PURA, PWP2, QRICH1, RAB33B, RANBP10, RANBP6, RASA1, RBBP6, |
| | | RBM10, RBM12, RBM19, RBM4, RBM4B, RBM6, RECK, RGL2, RGS14, RIN3, RLF, RNASET2, |
| | | RNF111, RNF125, RNF141, RNF41, RPARP-AS1, RPL11, RPL14, RPL18, RPL22, RPL24, RPL27, |
| | | RPL31, RPL34, RPL35, RPL35A, RPL39, RPL6, RPL7, RPS12, RPS16, RPS17, RPS21, RPS23, |
| | | RPS25, RPS27A, RPS28, RPS29, RPS3, RPS6, RPS6KA3, RPS7, RRAGA, RRNAD1, RSAD1, |
| | | RSBN1, RXRB, RYK, S1PR1, SAFB, SAP18, SARAF, SAV1, SC5D, SDCBP, SDR39U1, SEC31B, |
| | | SELL, SENP6, SEPT6, SERTAD2, SESN1, SET, SETD2, SETDB1, SF3B1, SFPQ, SFSWAP, |
| | | SGPL1, SH2B3, SH3BGRL, SH3YL1, SIK3, SIN3B, SIRT1, SKP1, SLC23A2, SLC25A36, |
| | | SLC25A44, SLC37A1, SLC6A16, SLC7A6, SLC9A8, SMAD3, SMARCE1, SMIM7, SNN, SNRK, |
| | | SNUPN, SNX1, SNX11, SNX2, SNX27, SOCS5, SORL1, SOS2, SP140L, SPECC1L, SPG7, |
| | | SPSB3, SRRM2, SRSF5, SRSF6, SRSF7, SRSF8, SSBP2, SSH1, ST3GAL1, STAG2, STAT5A, |
| | | STAT5B, STK19, STK26, STX16, STX4, STX6, SUGP2, SYNE1, SYPL1, TAFIA, TAF1C, |
| | | TAF1D, TAF7, TAOK3, TARDBP, TAZ, TBC1D13, TBP, TCF3, TCF7, TCTN1, TGFBR2, |
| | | TGFBRAP1, TGIF1, TGIF2, TM2D3, TMCC1, TMCO6, TMEM134, TMEM164, TMEM2, |
| | | TMEM243, TMF1, TMUB2, TMX4, TNFAIP3, TNFRSF10B, TNKS, TNS3, TOM1, TP73-AS1, |
| | | TPCN1, TPT1, TRAF3, TRAF5, TRAK1, TRAPPC10, TRAPPC12, TRAPPC8, TRIM13, TRIM27, |
| | | TRIM33, TRIM44, TRIM68, TRMT61B, TSC1, TSC22D3, TSEN34, TTC12, TTC31, TTC9, TTF1, |
| | | TUBA1A, TUG1, TXNIP, TXNL4B, UBE2G2, UBE2I, UBQLN2, UBR2, UBR4, UBXN1, |
| | | UNC45A, USP12, USP24, USP7, UXS1, VAMP4, VEZF1, VILL, VPS11, VPS13D, VPS35, VPS39, |
| | | WASF1, WDR19, WDR37, WDR55, WDR59, WDR5B, WIPF2, WIPI2, WSB1, XPC, XPO6, |
| | | XRCC2, XYLT1, YPEL5, YTHDC1, YY1AP1, ZBED5, ZBTB11, ZBTB14, ZBTB40, ZBTB5, |
| | | ZBTB7A, ZC3HAV1, ZDHHC17, ZDHHC6, ZHX2, ZKSCAN5, ZMYM4, ZMYM5, ZNF106, |
| | | ZNF134, ZNF136, ZNF137P, ZNF14, ZNF195, ZNF204P, ZNF211, ZNF212, ZNF217, ZNF222, |
| | | ZNF230, ZNF232, ZNF235, ZNF24, ZNF248, ZNF266, ZNF268, ZNF274, ZNF277, ZNF302, |
| | | ZNF304, ZNF318, ZNF32, ZNF329, ZNF37BP, ZNF395, ZNF419, ZNF443, ZNF451, ZNF551, |
| | | ZNF592, ZNF606, ZNF672, ZNF767P, ZNF83, ZNF839, ZNF84, ZRSR2, ZSCAN18, ZSCAN32, |
| | | ZXDC, ZZEF1 |
| CD33 | Royalblue | ACTC1, ADAMTS2, ADD1, AGGF1, AGPAT1, ANXA9, APBB1IP, APLP2, ARHGDIA, |
| | | ARHGEF11, BCL2L1, C10orf76, C1orf115, CA5BP1, CACNB2, CFAP70, CLDN4, COCH, |
| | | COL8A2, CPSF6, CSNK1G1, CYB5R2, CYP1A2, DACH1, DBT, DEDD, DOCK5, DUSP7, E2F4, |
| | | ECE2, EPN1, ERCC8, ERV9-1, FADS2, FAM124B, FAM13A, FASN, FUT6, GNAQ, GTPBP1, |
| | | HFE, HIST1H2AM, HMGCS2, HTATSF1, IGLJ3, KCNJ3, KCNMB3, KLHL26, LDLRAD4, |
| | | LPAR1, MAP7, MATN1, MED14, MOB4, MVB12B, NDN, NREP, OPRL1, OR7E12P, PANK3, |
| | | PDLIM7, PDX1, PEX14, PLAA, PLCE1, PLSCR2, PNPLA2, PPARG, PPP2R1B, PRKCA, PSEN2, |
| | | PTCRA, PTGFR, PTK2B, PTPN1, PXMP4, RBKS, RERE, RITA1, SCN1B, SIGLEC7, SP1, |
| | | SPATA2, STAP2, STRN4, STYXL1, TBC1D10B, TDRD12, TMEM45A, ZHX3, ZNF155, ZNF235, |
| | | ZNF536, ZNF556 |
| CD33 | Sienna3 | ACSL1, ACVR1, ADM, AP2B1, APOL6, AQP9, ARID5B, ATP2A2, B3GALT4, BCAS2, C2, |
| | | CALU, CCL2, CCR1, CD38, CD63, CDS2, CHD7, CHST11, CLIC4, CSF2RB, CSNK1A1, CUL1, |
| | | CXCL8, DCTN5, DDX60, DESI1, DNASE2, DRAM1, EGR1, EMC1, EMC7, EPHB2, ERO1L, |
| | | ERP44, FCAR, FCGR1B, FFAR2, FLVCR2, GALNT2, GAS7, GK, GNAI3, GNPDA1, HPSE, |
| | | IER2, IFI27, IFI35, IFI6, IFIT2, IFIT3, IGSF6, IL15RA, IL1RN, IRF7, ISG20, JMJD6, JUN, |
| | | KCNJ15, KHNYN, KLF9, KMO, KYNU, LAMP1, LAP3, LDHA, LDLR, LEPROT, LGALS3BP, |
| | | LGALS8, LIMK2, LMNB1, LXN, MAPK1IP1L, MED6, MR1, MT1HL1, MT1X, MT2A, N4BP1, |
| | | NAIP, NAMPT, NAPA, NMI, NRAS, NUDT15, OASL, PANX1, PEF1, PLIN3, PLSCR1, POP4, |
| | | PPP1R2, PRPF18, PSMD12, PSMD14, QKI, RAB5A, RAB8B, RIPK2, RIT1, RNF19B, RSAD2, |
| | | SC5D, SEC24A, SERPING1, SIRPA, SLC12A6, SLC2A3, SLC31A2, SLC38A2, SLC39A8, |
| | | SPATS2L, SQLE, SREBF2, SRP54, STAT1, SUMO3, SYNJ2, TAP1, TCEB1, TFEC, TMCO1, |
| | | TMEM180, TMSB10, TNFAIP6, TOR1B, TRAFD1, TRIP4, TXN, XBP1, YME1L1, ZDHHC3 |
| CD33 | Violet | ACOT13, ACOX1, ACPP, ACTA2, ADAR, ADARB1, AIM2, APOBEC3G, ATP10A, BCL2L13, |
| | | BLM, BLVRA, BLZF1, C15orf39, C19orf66, C1QA, C2orf47, CACNA1A, CBR1, CD2AP, CDK7, |
| | | CEBPG, CHMP5, CHST12, CLU, CMC2, CMTR1, CNIH4, CNP, COA3, COX17, CR1, CSNK1D, |
| | | CYB5A, DBI, DDA1, DDX58, DHX58, DHX8, DNAJA1, DPM1, DYNLT1, DYSF, EIF2AK2, |
| | | ENOPH1, ERGIC2, ETFDH, ETNK1, EXT1, EXT2, F2RL1, FAM49A, FAM69A, FAM8A1, FAS, |
| | | FKBP15, FOLR3, FXYD6, GCH1, GLE1, GMPR, GRK6, GRPEL1, GTPBP2, HERC5, HERC6, |
| | | HGF, HINFP, HIST2H2BE, HSPB11, IFI44, IFI44L, IFIH1, IFIT1, IFIT5, IFITM1, IFITM3, IGJ, |
| | | IGKC, IGLC1, IK, ING1, IRF2, ISG15, JUP, KEAP1, KIAA0040, KIAA0226, KLHL7, LAIR2, |
| | | LARP7, LEPROTL1, LILRA2, LILRA5, LOC100996756, LY6E, LY96, MAD2L1BP, MCTS1, |
| | | MICU1, MRPL22, MSRB2, MX1, MX2, NCALD, NDFIP1, NDUFS6, NFYA, NRN1, NSUN3, |
| | | NUCB1,NUDT9, OAS2, OAS3, OSBPL1A, PAK2, PDK3, PGGT1B, PHF11, PML, POLB, |
| | | PPP1R3D, PPP2R2A, PSMA4, PSME3, QRSL1, RBMS2, RIN2, RNF34, RPS6KC1, RTP4, |
| | | SAMD4A, SAMD9, SAP30L, SCCPDH, SEC22B, SIGLEC1, SLC25A37, SMAD3, SMCHD1, |
| | | SNRPG, SNTB1, SORT1, SP100, SP110, SP140, SPATA5L1, SPTLC2, SRD5A1, SRP19, STAP1, |
| | | STEAP4, STX17, SULT1B1, TBPL1, TCF4, TCN2, TDRD7, TLE3, TMEM140, TMEM2, |
| | | TMEM255A, TMOD3, TNFSF10, TRIM14, TRIM21, TRIM22, TROVE2, UAP1L1, UBE2K, |
| | | USP18, VAMP1, VTI1B, WDFY3, WWC3, XRCC4, ZNF322 |
| CD33 | Darkmagenta | ABCC4, ACTR1B, AHI1, AK1, AKAP13, AKAP8L, ALG12, ANGEL1, ANK3, ANKEF1, APOM, |
| | | AQP3, ARFIP2, ARFRP1, ARHGEF16, ARRB1, ARTN, ASAP3, ATAD3A, ATN1, B3GAT1, |
| | | B4GALT1, BAHD1, BATF3, BCL2, BCL7A, BIN1, BOP1, BTBD2, C10orf2, C14orf1, C16orf45, |
| | | C16orf58, C5orf45, CA11, CABP1, CACNA1I, CACNA2D2, CASP10, CD5, CD74, CD79A, |
| | | CDHR1, CDK16, CEP170B, CES2, CFHR2, CIC, CLEC10A, CLOCK, CLUAP1, CMTM6, |
| | | CNTFR, COL1A2, COMT, COQ3, COQ4, COQ7, COX11, CRELD1, CRTAC1, CRY2, CRYGD, |
| | | CRYM, CSF1R, CTSF, CWF19L1, CXADR, CYLD, DAO, DDX31, DECR2, DIEXF, DMPK, |
| | | DNPEP, DNPH1, DOCK6, DOLPP1, DPH2, DSPP, DTNA, DZIP3, ECHS1, EHD2, EIF3A, EIF5A, |
| | | ENPP1, EPOR, ERBB2, ESR2, EVX1, EXOSC4, FAM153A, FANCC, FBXO2, FBXO31, FCF1, |
| | | FGF4, FGF6, FHL1, FKBP4, FUBP1, FZR1, GAMT, GDF5, GFER, GJB3, GLP1R, GNB1L, |
| | | GOLGA2P5, GOLGA8A, GON4L, GRHL2, HABP4, HADH, HEMK1, HGH1, HIP1R, HIST1H1T, |
| | | HIST3H2A, HLA-DPA1, HLA-DQB1, HMGA1, HMOX2, HNRNPA0, HRK, ICAM4, IGH, |
| | | IGSF9B, IPO9, ISYNA1, IZUMO4, KCNA3, KDM8, KLF12, KLHDC3, LIMD2, LIMS2, |
| | | LINC00260, LINC01278, LRRC16A, LTA, LTBP4, MACROD1, MAZ, MCM3AP, MCM3AP-AS1, |
| | | MEGF6, MGAT4B, MID2, MMP19, MRM1, MRPL12, MRTO4, MUC5AC, MUC8, MYO1C, |
| | | NAA40, NECAB3, NF2, NFATC1, NFATC2IP, NIPAL3, NIPSNAP1, NPAT, NPM3, NPRL3, |
| | | NPTXR, NR3C2, NRF1, NRL, NSG1, NUBPL, NUFIP1, OSBP2, PCBP4, PCYT2, PDLIM4, |
| | | PGAP2, PHC1, PHGDH, PHLPP2, PIK3IP1, PLCG1, PLCH2, PLXNB2, PNPLA4, POF1B, |
| | | POLD4, POLR3G, POU6F1, PPDPF, PPIP5K1, PPP1R13B, PPP2R5D, PREPL, PSAT1, PTGIR, |
| | | RAB11FIP3, RAB2A, RAB40B, RBM19, RCL1, REXO4, RFTN1, RGS12, RNPS1, ROBO3, |
| | | RPS12, RRP1B, SAFB2, SBF1, SEMA3G, SF3B3, SGSM2, SH2D3A, SIVA1, SLC12A2, |
| | | SLC25A22, SLC25A4, SLC2A6, SLC5A5, SLC7A8, SMPD2, SNAP25, SOX12, SPDEF, SPTBN1, |
| | | SREK1IP1, SSBP3, STAG3, STRA13, SURF2, SYNGR3, TAC3, TCEA2, TCEB2, TCL6, TEAD3, |
| | | TFAM, TJP3, TLE2, TM7SF2, TMEM177, TMEM63A, TOP3B, TPT1P8, TRAF3IP3, TRAF4, |
| | | TRAK1, TRIM2, TSKU, TSPAN5, TTC28, TUBD1, TULP3, UBE2D4, UBE2O, USP13, USP5, |
| | | UTP20, VASH1, VPS13D, WDR59, WDR61, WDR73, WDR74, YPEL1, ZBTB38, ZFP36L2, |
| | | ZNF510, ZNF76, ZSCAN18 |
| LDG | LDG_A | ABCC3, ABCC4, ABHD15, ABI2, ABLIM3, ACER3, ACRBP, ACSBG1, ACVR1, ADCY3, |
| | | ADRA2A, AFAP1, AFAP1L2, AFF3, AGBL5, AGPAT5, AIG1, AKIP1, ALDH1A1, ALOX12, |
| | | ANKRD28, ANO6, AP1S2, APP, AQP10, AR, ARHGAP18, ARHGAP21, ARHGAP32, |
| | | ARHGAP6, ARHGEF12, ARMCX3, ASAP2, ATP5E, ATP5S, ATP9A, AVPR1A, B4GALT6, |
| | | BACE1, BCL11A, BCL2L1, BCL2L2, BEND2, BET1, BEX3, BICD1, BLNK, BMP6, BMP8B, |
| | | C12orf75, C12orf76, C15orf52, C15orf54, C19orf33, C1orf198, C2orf88, C7orf73, CA13, CA2, |
| | | CALD1, CAMTA1, CANX, CASP6, CCDC88A, CD151, CD226, CD36, CDC14B, CDIP1, |
| | | CDK2AP1, CDK6, CDKL1, CDYL, CHD9, CLCN3, CLDN5, CLEC1B, CLIC4, CLU, CMTM5, |
| | | CNRIP1, CNST, COMT, CPED1, CPNE5, CRAT, CRLS1, CTC-338M12.4, CTDSPL, CTTN, |
| | | CXCL5, DAAM1, DAB2, DCLRE1A, DDX11L2, DENND2C, DIMT1, DMTN, DNAJC6, DNM3, |
| | | DPPA4, DPYSL2, DST, EGF, EGLN3, EHD3, ELOVL7, ENDOD1, ENKUR, EPB41L3, ERG, |
| | | ERV3-1, ESAM, F13A1, F2R, FAM20B, FAM212B-AS1, FAM65C, FAM69B, FAM81B, |
| | | FAXDC2, FHL1, FHL2, FKBP1B, FNBP1L, FRMD3, FSTL1, GADD45A, GAS2L1, GGTA1P, |
| | | GLCE, GMPR, GNA12, GNAZ, GNG11, GNG8, GP1BA, GP5, GP6, GPX1, GRAP2, GRB14, |
| | | GSTP1, GUCY1A3, GUCY1B3, H1F0, H2AFJ, HEMGN, HEXIM2, HGD, HIST1H2AE, |
| | | HIST1H2BJ, HIST1H2BO, HIST1H4I, HMGB1, HMGN1, HRASLS, IGF2BP3, IGKC, IGLC1, |
| | | IRS1, ITGA2B, ITGA9, ITGB1, ITGB3, ITGB5, JAM3, KALRN, KCND3, KIF2A, KLHL5, |
| | | LAPTM4B, LGALSL, LINC00853, LINC00938, LIPH, LMNA, LOC101928419, LOC105371967, |
| | | LOC105377276, LOC283194, LPAR5, LRBA, LTBP1, LYPLAL1, LZTS2, M1AP, MAGI2-AS3, |
| | | MAGOHB, MAP1A, MAP1B, MAP3K7CL, MAST4, MAX, MBTD1, MCM6, MCUR1, MEIS1, |
| | | MEST, MFAP3L, MGLL, MINPP1, MITF, MLH3, MMD, MMRN1, MOB1B, MPL, MSANTD3, |
| | | MSN, MTHFD2L, MTMR2, MTURN, MYB, MYCT1, MYL9, MYLK, MYNN, NAP1L1, NAT8B, |
| | | NCAPG2, NCK1-AS1, NCKAP1, ND4, NENF, NEXN, NIPA1, NLK, NORAD, NPRL3, NREP, |
| | | NRGN, NT5M, NUTM2A-AS1, OPN3, P2RY12, PANX1, PARD3, PARVB, PAWR, PBX1, |
| | | PCYT1B, PDE2A, PDE3A, PDE5A, PDGFA, PDGFC, PDLIM1, PDZD2, PDZK1IP1, PEAR1, PF4, |
| | | PF4V1, PGRMC1, PITPNM2, PKHD1L1, PKIG, PLA2G12A, PLEKHA8P1, PLOD2, PNMA1, |
| | | PPBP, PPM1L, PRDX6, PRG2, PRKAR2B, PROS1, PROSER2, PRTFDC1, PRUNE1, PSD3, |
| | | PSPH, PTCRA, PTGIR, PTGS1, PTK2, PTPN18, PTPRS, PXDC1, PYGB, RABB, RAB27B, |
| | | RAB30, RAP1B, RAP2B, RBPMS2, RCC2, RDH11, RGS10, RHBDD1, RHOBTB1, RNF11, |
| | | RNF217, RSU1, SAV1, SCFD2, SCN9A, SDC4, SDPR, SEC14L5, SEPT11, SERPINE2, |
| | | SH3BGRL2, SH3TC2, SHTN1, SIAE, SLA2, SLC25A43, SLC35D2, SLC35D3, SLC44A1, |
| | | SLC8A3, SMAD1, SMIM24, SMIM5, SMOX, SNAPC3, SNCA, SNPH, SOX4, SPARC, SPOCD1, |
| | | SPSB1, SPX, SSX2IP, ST3GAL3, STMN1, STON2, STRADB, SYNM, SYTL4, TAL1, TARBP1, |
| | | TBXA2R, TCEAL8, TCF4, TCL1A, TDRP, TEX2, TFB1M, TFPI, TGFB1I1, TGFBI, THBS1, |
| | | THRB, TLK1, TLR7, TMCC2, TMEM158, TMEM40, TMEM45A, TMEM64, TNFSF4, TNIK, |
| | | TNS1, TNS3, TPM1, TPSAB1, TPSB2, TPST2, TPTEP1, TRBV27, TREML1, TRIM10, TRIM13, |
| | | TRIM58, TSC22D1, TSPAN18, TSPAN33, TSPAN9, TTC7B, TUBB, TUBB1, TWSG1, UBE2E2, |
| | | UBE2O, UBL4A, UGCG, USP12, USP31, UXS1, VCL, VEPH1, VIL1, VSIG2, VWA5A, VWF, |
| | | WASF1, WASF3, WDR11-AS1, WHAMMP2, WRB, WWC1, XK, XPNPEP1, YIF1B, YWHAE, |
| | | YWHAH, ZBTB16, ZC3HAV1L, ZNF175, ZNF271P, ZNF367, ZNF431, ZNF521, ZNF529-AS1, |
| | | ZNF542P, ZNF677, ZNF718 |
| LDG | LDG_B | ABCA13, ARG1, ATP8B4, AZU1, CAMP, CEACAM6, CEACAM8, CHIT1, CLEC12A, CLEC5A, |
| | | CPNE3, CRISP3, CTSG, CYBB, DEFA4, ELANE, HP, LCN2, LTF, MGST1, MMP8, MPO, |
| | | MS4A3, OLFM4, OLR1, RNASE3, SERPINB10, SLC2A5, STOM, TCN1, ANLN, BIRC5, BUB1B, |
| | | CCNA1, CDK1, CDKN2B, DHFR, GFI1, INHBA, IQGAP3, KIAA0101, KIF11, KIF14, KNL1, |
| | | MIS18BP1, NCAPG, RGCC, RRM2, SKA2, TOP2A, TYMS, AGPS, ANXA4, ATP23, BCL2L15, |
| | | BEX1, CD24, CTBP2, CTC1, DCBLD2, ECRP, ERG, FBXO9, GALNT10, GCLM, GLOD5, |
| | | GVINP1, HMGB2, HMGN2, KBTBD6, LINC00323, LMO4, MED7, NFYC, NUCB2, PCOLCE2, |
| | | PDLIM5, PLEKHA3, PPFIA4, RPE, SCD, SENP1, SLC28A3, SMIM8, TACSTD2, TCTEX1D1, |
| | | THBS4, TMEM234, TMEM50B, TMLHE, TRMT5, ZNF788 |
| PC | PC_Up | AAK1, ADA, ADCYAP1, ADGRB1, AGK, AHCYL2, ALG5, ALG9, AMOTL2, ANG, ANKS1B, |
| | | APOA4, AQP3, ARF4, ARHGEF40, ARL1, ASIC1, ASPM, ATF5, ATP11A, ATP1A2, ATP2A2, |
| | | AURKA, B4GALT3, B9D1, BAZ1B, BCAN, BIK, BIRC5, BMP8B, BSCL2, BUB1, BUB1B, |
| | | C11orf80, C1GALT1C1, C1orf27, CA6, CADM1, CADM3, CALML4, CALR, CALU, CASP3, |
| | | CAV1, CCNA2, CCNB1, CCNB2, CCNC, CCND2, CCNE2, CCR10, CD27, CD300A, CD320, |
| | | CD38, CD59, CD6, CDC20, CDC25A, CDC42BPA, CDC6, CDCA3, CDKN2C, CDKN3, CDR2, |
| | | CENPE, CENPN, CENPU, CEP55, CEP97, CFLAR, CHAC1, CHEK1, CHPF, CHST12, CHST2, |
| | | CITED2, CKAP4, CLIC3, CLINT1, CNKSR1, CNPY2, COL9A3, COPA, COPB2, COX11, |
| | | COX7A2, CRB1, CRELD2, CSF2RB, CSHL1, CSNK1E, CTNNAL1, CYP11B2, CYP26A1, |
| | | CYP2E1, DCPS, DDOST, DENND1B, DERL1, DERL2, DLGAP5, DNAJC1, DNAJC3, DOK4, |
| | | DRD4, DSTN, E2F8, EDEM2, EDEM3, EFS, ELL2, ERAP1, ERCC6L, ESPL1, ESR1, EXOSC4, |
| | | EXT1, FAAH, FABP5, FAM149A, FAR2, FAXDC2, FBXO5, FDX1, FEN1, FKBP11, FKBP2, |
| | | FNDC3B, FOLH1B, FUT8, FZD7, GAB1, GADD45A, GALNT2, GARS, GAS6, GC, GCSH, GFI1, |
| | | GGH, GLRX5, GMNN, GMPPA, GMPPB, GNAS, GOLT1B, GPLD1, GPR15, GPRC5D, GRIK1, |
| | | GSC2, GSPT1, H2AFX, HDLBP, HIBCH, HIST1H2AM, HIST1H2BB, HIST1H2BC, HIST1H2BG, |
| | | HIST1H3D, HIST1H4B, HIST1H4L, HJURP, HMGN5, HMMR, HPGD, HPX, HRH1, HSD11B2, |
| | | HSD17B8, HSP90B1, HSPA13, HSPA5, HYOU1, IDH2, IFNAR2, IGF1, IGHD, IGHG1, IGHG3, |
| | | IGHM, IGK, IGKC, IGKV1-5, IGKV1D-13, IGKV1D-8, IGL, IGLJ3, IGLL1, IGLL3P, IGLV3-19, |
| | | IGLV4-60, IL1R1, IL6R, IL6ST, INPP4A, IQGAP2, IQSEC2, IRF4, ITGA6, ITGB1BP1, ITM2C, |
| | | JCHAIN, KCNJ5, KCNK12, KCNN3, KDELC1, KDELR2, KIAA0101, KIF20A, KIFC1, KIR2DL4, |
| | | KLF10, KLK11, KLKB1, LAP3, LAX1, LDLRAD4, LGALS3, LIME1, LMAN1, LMAN2, |
| | | LRRC59, LSR, LZTS1, MAN1A1, MANEA, MANF, MAP2K6, MAPKAPK5, MAST1, MBNL2, |
| | | MCM10, MCM3AP, MCUR1, MELK, MGAT2, MIF, MKI67, MLEC, MORF4L2, MPHOSPH9, |
| | | MRPL22, MTNR1A, MTRR, MUC5B, MYCBP, MYDGF, MYO1D, NANS, NAT2, NAT8, |
| | | NCAPG, NCOA3, NDUFB6, NEK2, NES, NEU1, NEUROG3, NME1, NPIP, NPIPB15, NPM1, |
| | | NT5DC2, NUCB2, NUS1P3, NUSAP1, OGFOD3, OGT, P4HB, PAK5, PAM, PARP2, PCDHGA3, |
| | | PCSK4, PDE1A, PDIA2, PDIA4, PDIA6, PDK1, PDXK, PERP, PGM3, PHGDH, PIK3CG, PKP4, |
| | | PMM2, POU6F2, PPA1, PPCDC, PPIB, PRDM1, PRDX4, PREB, PROSC, PSMA3, PSMC2, |
| | | PTPRD, PTTG1, PTTG3P, PYCR1, PYCRL, R3HCC1, RAB27A, RAPGEF2, RBM47, RGS13, |
| | | RGS16, RPN1, RPN2, RRBP1, RRM2, RS1, RWDD2A, SAR1A, SAR1B, SCUBE3, SDC1, |
| | | SDF2L1, SEC13, SEC14L1, SEC23B, SEC24A, SEC24D, SEC61A1, SEC61B, SEC61G, SEL1L, |
| | | SELPLG, SEMA4A, SEPT10, SEPT4, SERPINF1, SGK1, SIL1, SLAMF7, SLC16A1, SLC16A6, |
| | | SLC19A1, SLC1A4, SLC1A7, SLC27A2, SLC31A2, SLC35B1, SLC7A11, SLC7A5, SLC9A3R1, |
| | | SLCO2B1, SLCO3A1, SLCO4A1, SLFN12, SMAD6, SPATS2, SPCS1, SPCS2, SPCS3, SPINK5, |
| | | SPRR1A, SRM, SRP19, SSR1, SSR3, SSR4, ST3GAL6, ST6GALNAC4, STARD5, STT3A, |
| | | SULT1C2, TAZ, TBL2, TECR, TIMM17A, TIMM44, TIMM8B, TIMP2, TIMP4, TK1, TLX3, |
| | | TM9SF1, TMBIM6, TMED10, TMED2, TMED5, TMEM184B, TMEM208, TMEM258, |
| | | TNFRSF17, TP63, TPP2, TPST2, TRA, TRAM1, TRAM2, TRAT1, TRD, TRIB1, TRIP13, TRIP6, |
| | | TSHR, TST, TUBG1, TXN, TXNDC15, TXNDC5, TYMS, UAP1, UBE2C, UCHL1, UCK2, |
| | | UGGT2, UQCRB, VDR, VEGFA, WARS, WHSC1, WIPI1, XBP1, XCL1, YIPF2, ZMYM2, |
| | | ZNF593, ZWINT |
| PC | PC_Down | ABLIM1, ABR, ADARB1, AKAP1, AKT3, ALOX5AP, ANKZF1, ARHGAP17, ARPC4, BANK1, |
| | | BCL11A, BIN1, BLK, BMP2K, C7orf26, CACNA1A, CAPN3, CBR3, CBX7, CCND3, CCR6, |
| | | CD19, CD1C, CD1D, CD22, CD37, CD72, CDK5R1, CEP170, CERS4, CIITA, CLCN4, CLIP2, |
| | | CNPPD1, COA1, CPQ, CSGALNACT1, CYLD, DCUN1D4, DDX24, DDX60, DEK, DENND5A, |
| | | DHX58, DPEP2, DYRK2, ELF4, FAM208A, FAM20B, FAM46A, FAM65A, FCGR2A, FCMR, |
| | | FGR, FOXO4, FYN, GAS7, GCNT1, GGA1, GGA2, GPD1L, GPR18, GRAP, GSAP, HCK, HHEX, |
| | | HIP1R, HLA-DMA, HLA-DMB, HLA-DOB, HLA-DPA1, HLA-DQB1, HLA-DRB1, HLA-DRB3, |
| | | HS3ST1, ID3, INPP5D, IRF5, IRF8, ITPKB, KDM4B, KIAA0141, KIF21B, KLF9, KLHDC10, |
| | | KMO, LAIR1, LAPTM5, LAT2, LBH, LINC00472, LIPA, LPGAT1, LYL1, LYST, MAPRE2, |
| | | MFHAS1, MNDA, MS4A1, MTSS1, MZF1, NAIP, NCR3, NLRP1, NOTCH2, NOTCH2NL, NT5E, |
| | | OPN3, P2RX5, PAX5, PCDH9, PDE4DIP, PDLIM2, PHC1, PIK3CD, PIKFYVE, PKIG, PLAC8, |
| | | PLCB2, PLEKHA1, PLEKHO1, POLD4, PPM1F, PRPF6, PRRC2B, PTK2, PTK2B, PTPN12, |
| | | PTPN6, PTPRCAP, RASGRP2, RBMS1, RIN3, RNF130, RNF141, RTL1, SAMD4A, SH3BP2, |
| | | SIDT2, SIPA1L1, SLC15A3, SMG1, SNAP23, SNN, SNX1, SNX2, SNX6, SPIB, SSBP2, STAT6, |
| | | STX7, SUSD5, SWAP70, SYNPO, SYPL1, TBC1D22A, TBL1X, TGFBR2, TMEM127, TNFSF12, |
| | | TNFSF13, TRAK1, TRAK2, TRIM34, TRIM38, TSPAN-3, TTC9, UNC119, UNKL, USF2, VAV3, |
| | | VEGFB, WASF2, XIST, ZBTB18, ZEB2, ZNF236, ZNF318, ZNF395, ZNF443, ZNF83, ZSCAN18, |
| | | ZXDC |

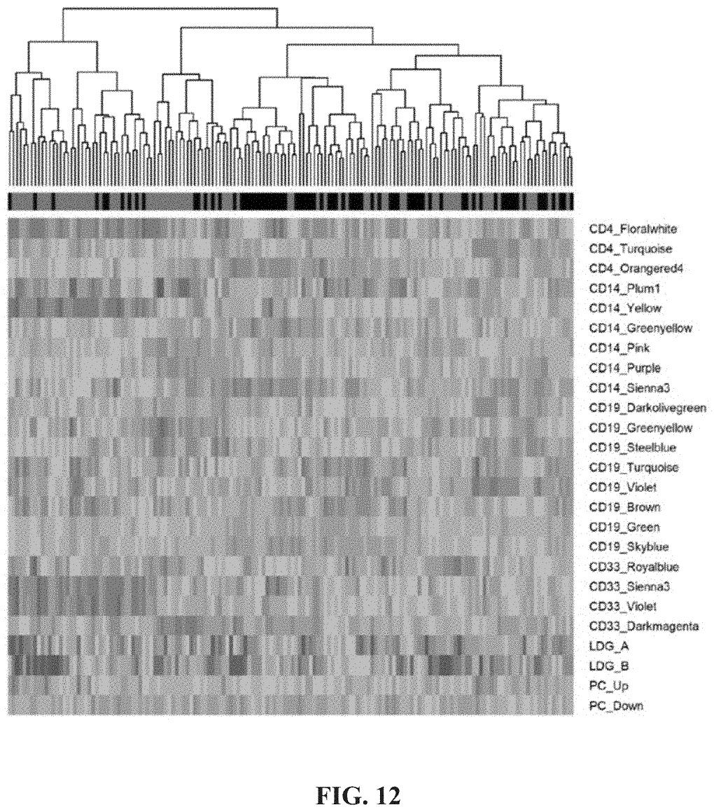

To characterize the relationships between SLE gene modules from cell subsets and disease activity in greater detail, Gene Set Variation Analysis (GSVA) enrichment was carried out using the 25 cell-specific gene modules (FIG. 12). Of the 25 cell-specific modules, 12 had enrichment scores with significant Spearman correlations to SLEDAI (p<0.05), and 14 had enrichment scores with significant differences between active and inactive patients (Welch's t-test, p<0.05) (Table 9). Table 9 shows assessment of WGCNA module relationships with SLE disease activity in WB, including statistics on WGCNA module relationships with SLEDAI and active disease. Correlation to SLEDAI was done by Spearman rank correlation, and the relationship with active versus inactive disease was assessed by Welch's unequal variances t-test and Cohen's d. Significant results are bolded (p<0.05). LDG: low-density granulocyte; PC: plasma cell.

TABLE 9
Cell-specific modules by Spearman correlation to SLEDAI and active vs. inactive state

| | Spearman correlation to SLEDAI | | Active vs. Inactive t-test | | |
|---|---|---|---|---|---|
| | rho | p value | t statistic | p value | d |
| CD4_Floralwhite | 0.360 | 3.90E−06 | 4.90 | 2.40E−06 | 0.788 |
| CD4_Turquoise | −0.044 | 0.587 | −0.93 | 0.352 | −0.149 |
| CD4_Orangered4 | −0.400 | 2.21E−07 | −5.29 | 4.35E−07 | −0.853 |
| CD14_Plum1 | 0.010 | 0.904 | −0.35 | 0.729 | −0.054 |
| CD14_Yellow | 0.356 | 4.93E−06 | 4.76 | 4.44E−06 | 0.761 |
| CD14_Greenyellow | −0.132 | 0.100 | −2.10 | 0.037 | −0.339 |
| CD14_Pink | −0.026 | 0.751 | 0.13 | 0.894 | 0.021 |
| CD14_Purple | −0.149 | 0.064 | −1.65 | 0.101 | −0.263 |
| CD14_Sienna3 | −0.368 | 2.27E−06 | −4.99 | 1.62E−06 | −0.799 |
| CD19_Darkolivegreen | 0.020 | 0.809 | −0.06 | 0.953 | −0.010 |
| CD19_Greenyellow | 0.192 | 0.016 | 2.55 | 0.012 | 0.403 |
| CD19_Steelblue | 0.016 | 0.838 | 0.55 | 0.580 | 0.089 |
| CD19_Turquoise | −0.069 | 0.393 | −0.84 | 0.403 | −0.132 |
| CD19_Violet | −0.087 | 0.282 | −1.48 | 0.141 | −0.236 |
| CD19_Brown | −0.050 | 0.537 | −1.04 | 0.301 | −0.164 |
| CD19_Green | −0.150 | 0.062 | −2.07 | 0.040 | −0.330 |
| CD19_Skyblue | −0.205 | 0.010 | −2.35 | 0.020 | −0.378 |
| CD33_Royalblue | 0.308 | 8.99E−05 | 3.99 | 1.03E−04 | 0.637 |
| CD33_Sienna3 | 0.362 | 3.41E−06 | 4.69 | 6.15E−06 | 0.753 |
| CD33_Violet | 0.322 | 4.15E−05 | 4.35 | 2.46E−05 | 0.696 |
| CD33_Darkmagenta | −0.216 | 6.74E−03 | −2.34 | 0.021 | −0.369 |
| LDG_A | −0.044 | 0.588 | −0.25 | 0.802 | −0.040 |
| LDG_B | 0.220 | 5.71E−03 | 2.37 | 0.019 | 0.377 |
| PC_Up | 0.262 | 9.75E−04 | 3.21 | 1.61E−03 | 0.508 |
| PC_Down | 0.022 | 0.781 | 0.80 | 0.426 | 0.129 |

Notably, each cell type produced at least one module with a significant correlation to SLEDAI in WB and at least one module with a significant difference in enrichment scores between active and inactive patients, demonstrating a relationship between disease activity in specific cellular subsets and overall disease activity in WB. However, the Spearman's rho values ranged from −0.40 to +0.36, suggesting that no one module had substantial predictive value. Furthermore, the effect sizes as measured by Cohen's d when testing active versus inactive enrichment scores ranged from −0.85 to +0.79. The CD4 Floralwhite and Orangered4 modules, which had the largest positive and negative effect sizes, respectively, showed a high degree of overlap in the enrichment scores of active and inactive patients (FIG. 4).

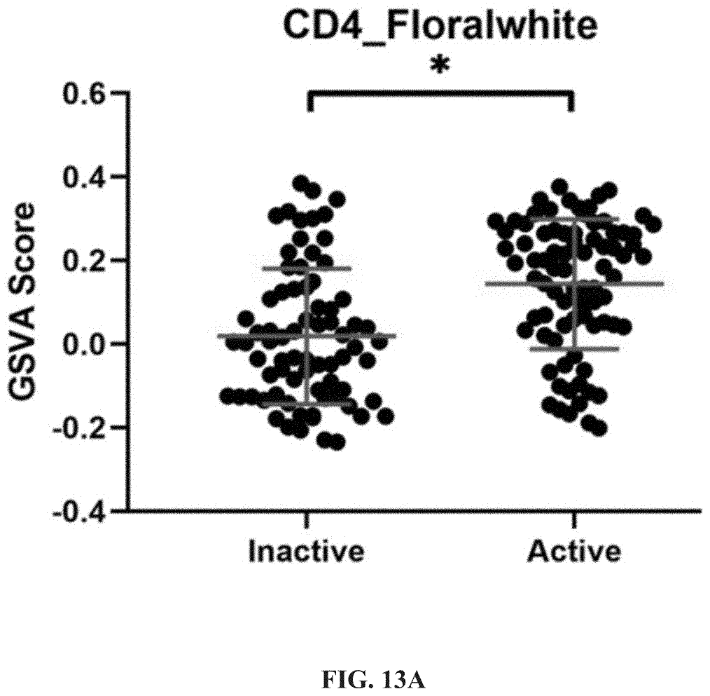

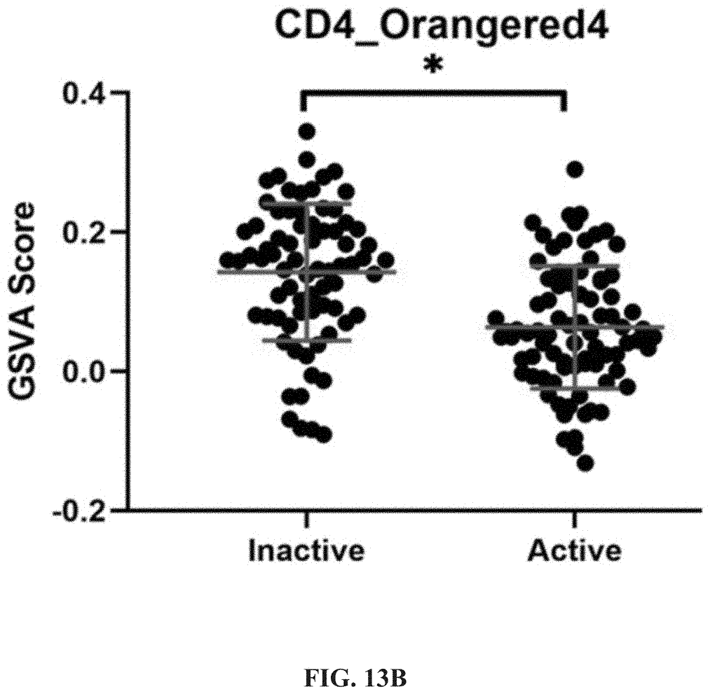

Analysis of individual disease activity-associated peripheral cellular subset gene modules was not sufficient to predict disease activity in unrelated WB data sets, since no single module from any cell type was able to separate active from inactive SLE patients (FIGS. 13A and 13B). The results emphasized the need for more advanced analysis to employ gene expression analysis to predict disease activity.

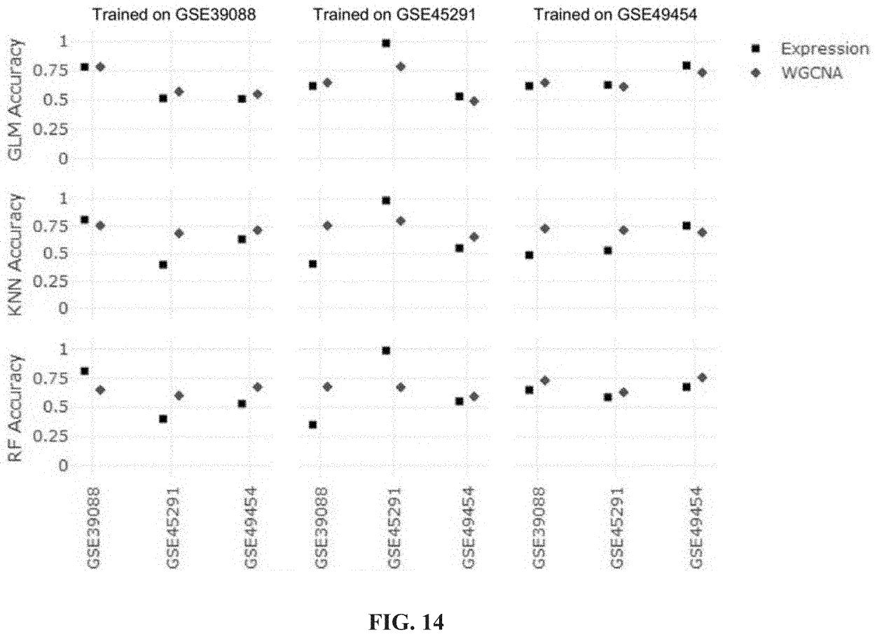

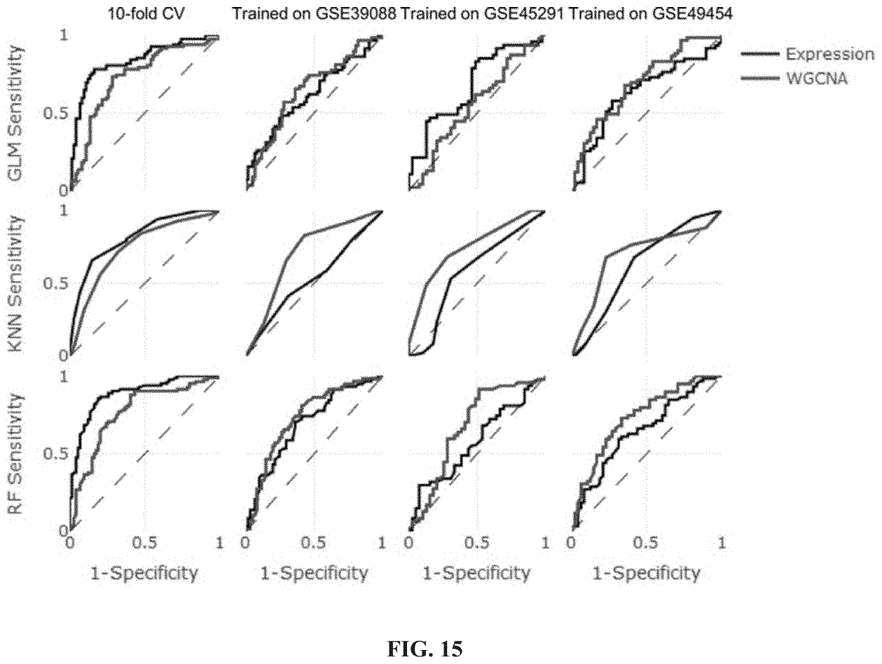

Machine learning may be applied to analyze and assess disease activity as follows. To assess the effectiveness of either raw gene expression or module-based enrichment techniques, SLE patients were classified as active or inactive using generalized linear models (GLM), k-nearest neighbors (KNN), and random forest (RF) classifiers. Classifiers were validated using two different methodologies: (1) 10-fold cross-validation or (2) study-based cross-validation, in which classifiers were trained on each data set independently and tested in the other two data sets. When evaluating the performance of classifiers on the data set on which they were trained, GLM accuracy was defined as one minus the cross-validated classification error from the cv.glmnet( ) function, and RF accuracy was determined based on out-of-bag predictions. The accuracy of each classifier trained with either gene expression or module enrichment is shown in FIG. 14, and receiver operating characteristic (ROC) curves are plotted in FIG. 15. Classification metrics for each classifier are shown in Table 10.

TABLE 10
Classification metrics for GLM, KNN, and RF classifiers

| | | 10-fold CV | | Trained on GSE39088 | | Trained on GSE45291 | | Trained on GSE49454 | |
|---|---|---|---|---|---|---|---|---|---|
| | | Expression | WGCNA | Expression | WGCNA | Expression | WGCNA | Expression | WGCNA |
| GLM | Accuracy | 0.80 | 0.72 | 0.51 | 0.56 | 0.57 | 0.56 | 0.63 | 0.63 |
| | Sensitivity | 0.78 | 0.73 | 0.86 | 0.79 | 0.51 | 0.60 | 0.54 | 0.59 |
| | Specificity | 0.82 | 0.70 | 0.18 | 0.34 | 0.64 | 0.51 | 0.73 | 0.67 |
| | AUC | 0.84 | 0.73 | 0.62 | 0.65 | 0.68 | 0.55 | 0.63 | 0.69 |
| | Kappa | 0.60 | 0.43 | 0.04 | 0.14 | 0.15 | 0.11 | 0.26 | 0.26 |
| | PPV | 0.83 | 0.73 | 0.50 | 0.53 | 0.63 | 0.60 | 0.71 | 0.69 |
| | NPV | 0.77 | 0.70 | 0.58 | 0.64 | 0.52 | 0.51 | 0.56 | 0.57 |
| KNN | Accuracy | 0.75 | 0.70 | 0.50 | 0.70 | 0.49 | 0.70 | 0.51 | 0.72 |
| | Sensitivity | 0.66 | 0.72 | 0.59 | 0.83 | 0.23 | 0.68 | 0.31 | 0.68 |
| | Specificity | 0.85 | 0.68 | 0.41 | 0.57 | 0.79 | 0.72 | 0.77 | 0.77 |
| | AUC | 0.82 | 0.74 | 0.54 | 0.71 | 0.58 | 0.75 | 0.63 | 0.70 |
| | Kappa | 0.50 | 0.40 | 0.00 | 0.40 | 0.03 | 0.40 | 0.07 | 0.44 |
| | PPV | 0.83 | 0.71 | 0.49 | 0.65 | 0.58 | 0.74 | 0.62 | 0.78 |
| | NPV | 0.69 | 0.68 | 0.51 | 0.78 | 0.46 | 0.65 | 0.47 | 0.66 |
| RF | Accuracy | 0.83 | 0.72 | 0.45 | 0.63 | 0.47 | 0.63 | 0.61 | 0.66 |
| | Sensitivity | 0.83 | 0.77 | 0.86 | 0.91 | 0.53 | 0.62 | 0.54 | 0.61 |
| | Specificity | 0.82 | 0.68 | 0.07 | 0.36 | 0.38 | 0.64 | 0.69 | 0.73 |
| | AUC | 0.89 | 0.77 | 0.69 | 0.73 | 0.58 | 0.68 | 0.65 | 0.74 |
| | Kappa | 0.65 | 0.45 | −0.07 | 0.27 | −0.08 | 0.26 | 0.22 | 0.33 |
| | PPV | 0.84 | 0.72 | 0.47 | 0.58 | 0.51 | 0.67 | 0.68 | 0.73 |
| | NPV | 0.81 | 0.72 | 0.33 | 0.81 | 0.41 | 0.58 | 0.55 | 0.60 |

When performing 10-fold cross-validation, the use of gene expression values resulted in better performance from all three classifiers compared to module enrichment scores. The random forest classifier was the strongest performer with 83 percent accuracy, and its corresponding ROC curve demonstrated an excellent tradeoff between recall and fall-out (AUC of 0.89). This high accuracy may likely be attributed to the presence of data from all three studies in both the training and test sets. In this case, the classifiers have the opportunity to learn patterns inherent to each data set, which proves useful during testing. To ensure that the classifiers were not disproportionately learning patterns from certain data sets at the expense of others, the classification results from the 10-fold cross-validation approach were subdivided by data set. All classifiers exhibited good performance with small differences between their highest and lowest accuracies in individual data sets, with the exception of the WGCNA-based KNN classifier (Table 11).

Table 11 shows classification metrics of 10-fold CV machine learning classifiers with results subdivided by data set. Data sets are listed by their GEO accession numbers. Range: difference between maximum and minimum values for each metric. Expression: gene expression data. WGCNA: module enrichment scores. AUC: area under the receiver operating characteristic curve. Kappa: Cohen's kappa coefficient. PPV: positive predictive value. NPV: negative predictive value.

TABLE 11
Classification metrics of 10-fold CV machine learning classifiers with results subdivided by data set

| | | Subset: GSE39088 | | Subset: GSE45291 | | Subset: GSE49454 | | Range | |
|---|---|---|---|---|---|---|---|---|---|
| | | Expression | WGCNA | Expression | WGCNA | Expression | WGCNA | Expression | WGCNA |
| GLM | Accuracy | 0.81 | 0.70 | 0.83 | 0.74 | 0.76 | 0.69 | 0.07 | 0.05 |
| | Sensitivity | 0.73 | 0.73 | 0.83 | 0.71 | 0.76 | 0.76 | 0.10 | 0.05 |
| | Specificity | 0.93 | 0.67 | 0.83 | 0.77 | 0.75 | 0.63 | 0.18 | 0.14 |
| | AUC | 0.85 | 0.74 | 0.84 | 0.75 | 0.84 | 0.70 | 0.01 | 0.05 |
| | Kappa | 0.63 | 0.39 | 0.66 | 0.49 | 0.51 | 0.39 | 0.15 | 0.10 |
| | PPV | 0.94 | 0.76 | 0.83 | 0.76 | 0.76 | 0.68 | 0.18 | 0.08 |
| | NPV | 0.70 | 0.63 | 0.83 | 0.73 | 0.75 | 0.71 | 0.13 | 0.10 |
| KNN | Accuracy | 0.78 | 0.84 | 0.76 | 0.70 | 0.71 | 0.59 | 0.07 | 0.25 |
| | Sensitivity | 0.68 | 0.86 | 0.71 | 0.71 | 0.56 | 0.60 | 0.15 | 0.26 |
| | Specificity | 0.93 | 0.80 | 0.80 | 0.69 | 0.88 | 0.58 | 0.13 | 0.22 |
| | AUC | 0.85 | 0.84 | 0.79 | 0.75 | 0.84 | 0.65 | 0.06 | 0.19 |
| | Kappa | 0.58 | 0.66 | 0.51 | 0.40 | 0.43 | 0.18 | 0.15 | 0.48 |
| | PPV | 0.94 | 0.86 | 0.78 | 0.69 | 0.83 | 0.60 | 0.16 | 0.26 |
| | NPV | 0.67 | 0.80 | 0.74 | 0.71 | 0.66 | 0.58 | 0.08 | 0.22 |
| RF | Accuracy | 0.81 | 0.81 | 0.83 | 0.71 | 0.84 | 0.67 | 0.03 | 0.14 |
| | Sensitivity | 0.82 | 0.82 | 0.86 | 0.74 | 0.80 | 0.76 | 0.06 | 0.08 |
| | Specificity | 0.80 | 0.80 | 0.80 | 0.69 | 0.88 | 0.58 | 0.08 | 0.22 |
| | AUC | 0.87 | 0.86 | 0.90 | 0.78 | 0.88 | 0.72 | 0.03 | 0.14 |
| | Kappa | 0.61 | 0.61 | 0.66 | 0.43 | 0.67 | 0.34 | 0.06 | 0.27 |
| | PPV | 0.86 | 0.86 | 0.81 | 0.70 | 0.87 | 0.66 | 0.06 | 0.20 |
| | NPV | 0.75 | 0.75 | 0.85 | 0.73 | 0.81 | 0.70 | 0.10 | 0.05 |

When performing study-based cross-validation, classifiers trained on expression data performed better on their respective training sets than those trained on module enrichment scores in nearly all cases (FIG. 14). However, the accuracy of classifiers trained on expression values in the test sets was approximately 50 percent. This is in line with the findings of the initial bioinformatic analysis (Table 6), namely, that gene expression values may have little utility when attempting to classify unfamiliar samples. When the training and test data come from different data sets, the classifiers learn patterns that are unhelpful for classifying test samples. Although classifiers trained on module enrichment scores did not achieve high accuracies in their training sets, they did not experience as sharp a drop in accuracy when tested on unfamiliar data sets. Remarkably, the use of module enrichment scores improved RF test accuracy to approximately 65 percent and improved KNN test accuracy to approximately 70 percent.

Overall, gene expression values provide high accuracy when performing 10-fold cross-validation but are rendered nearly useless when performing study-based cross-validation. These results indicate that disease activity classification based on raw gene expression, while more accurate, is sensitive to technical variability, whereas classification based on module enrichment better copes with variation among data sets.

Random forest consistently achieved high performance, and its assessments of variable importance may be used to gain insight into directors of the identification of SLE activity. To this end, random forest classifiers were trained on all patients from all data sets in order to identify the most important genes and modules as determined by mean decrease in the Gini impurity, a measure of misclassification error. The classifier trained with gene expression data achieved an out-of-bag accuracy of 81 percent, with a sensitivity of 83 percent and a specificity of 78 percent. The classifier trained with module enrichment scores achieved an out-of-bag accuracy of 73 percent, with a sensitivity of 78 percent and a specificity of 68 percent.

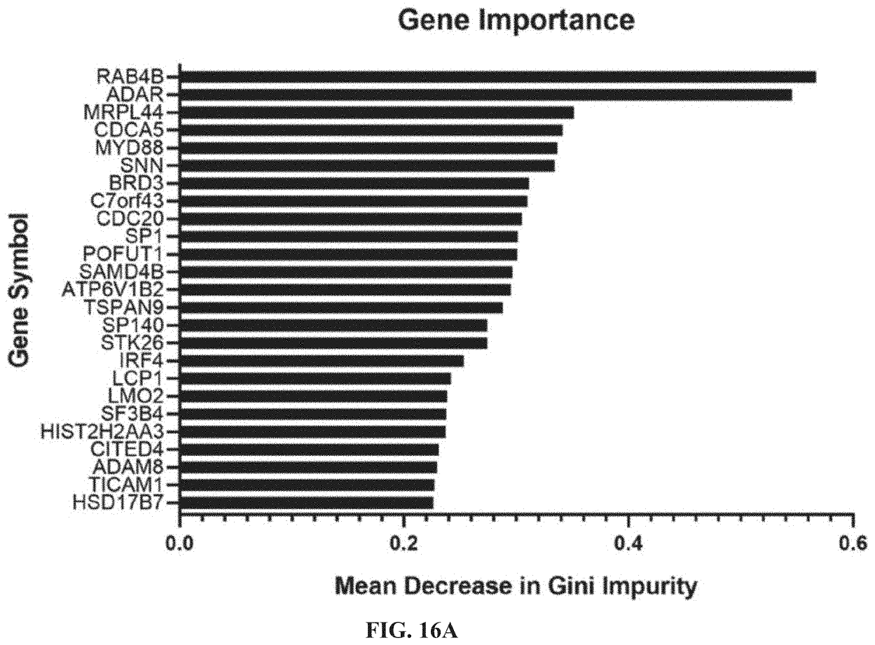

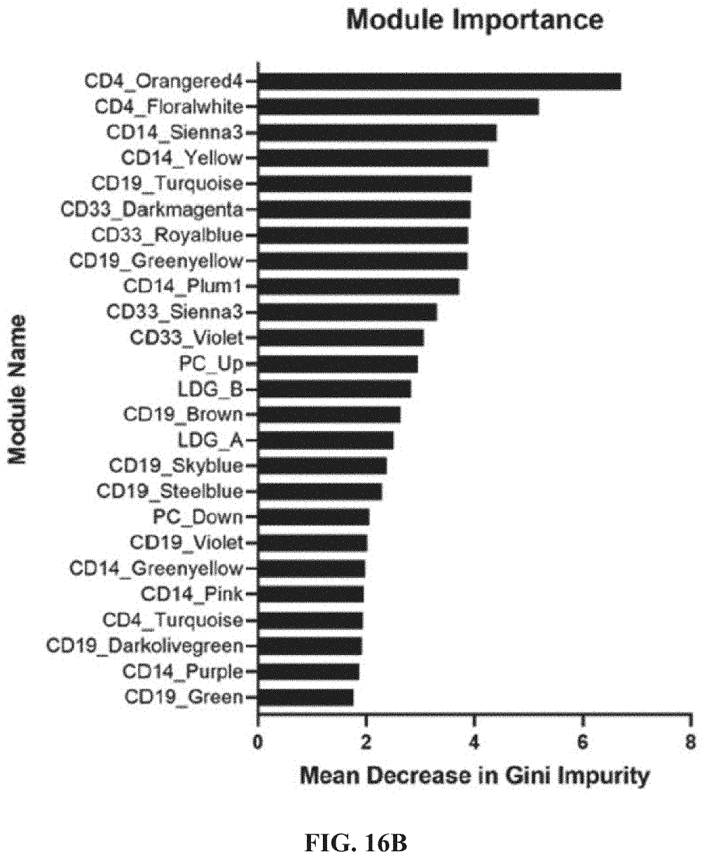

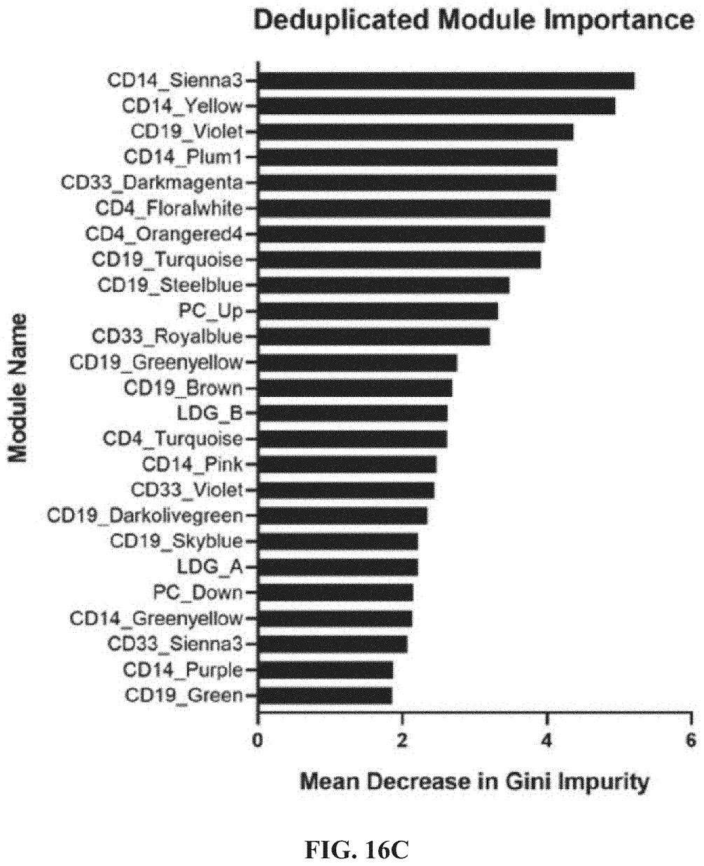

The most important genes and modules identified a wide array of cell types and biological functions (FIGS. 16A-16C). The most important genes encompass such diverse functions as interferon signaling, pattern recognition receptor signaling, and control of survival and proliferation (FIG. 16A). These most important genes include RAB4B, ADAR, MRPL44, CDCA5, MYD88, SNN, BRD3, C7orf43, CDC20, SP1, POFUT1, SAMD4B, ATP6V1B2, TSPAN9, SP140, STK26, IRF4, LCP1, LMO2, SF3B4, HIST2H2AA3, CITED4, ADAMS, TICAM1, and HSD17B7. Notably, the most influential modules skewed away from B cell-derived modules and towards T cell- and myeloid cell-derived modules (FIG. 16B). As some of these modules had overlapping genes, the variable importance experiment was repeated with modules that were de-duplicated by removing any genes that appeared in more than one module before GSVA enrichment scoring. The relative variable importance scores of the de-duplicated modules correlated strongly with those of the original modules (Spearman's rho=0.69, p=1.94E-4), indicating that module behavior was partly driven by the overlapping genes but strongly driven by unique genes (FIG. 16C).

CD4_Floralwhite and CD14_Yellow, two interferon-related modules which maintained high importance after deduplication, were further analyzed to study the effect of unique genes on module importance. Gene lists were tested for statistical overrepresentation of Gene Ontology biological process terms with FDR correction on pantherdb.org. CD4_Floralwhite did not show any significant enrichment, but CD14_Yellow, which had the highest importance after deduplication, was highly enriched for genes with the “Immune Effector Process” designation (26/77 genes, FDR=9.38E-11 by Fisher's exact test). This suggests that CD14+ monocytes express unique genes that may play important roles in the initiation of SLE activity.

Several important findings related to SLE gene expression heterogeneity within and across data sets have been elucidated by this study. First, DE analysis of active vs. inactive patients may be insufficient for proper classification of SLE disease activity, as systematic differences between data sets render conventional bioinformatics techniques largely non-generalizable.

Next, it was hypothesized that WGCNA modules created from the cellular components of WB and correlated to SLEDAI disease activity may improve classification of disease activity in SLE patients. The use of cell-specific gene modules based on a priori knowledge about their relevance to disease fared slightly better than raw gene expression, as it generated informative enrichment patterns, and many of the modules maintained significant correlations with SLEDAI in WB. However, these enrichment scores failed to separate active patients from inactive patients completely by hierarchical clustering.

Raw expression data was then compared alongside the WGCNA generated modules of genes in machine learning applications. A supervised classification approach was applied using elastic generalized linear modeling, k-nearest neighbors, and random forest classifiers. The trends in performance when cross-validating by study or cross-validating 10-fold indicate the potential advantages and disadvantages of diagnostic tests incorporating gene expression data or module enrichment. Cross-validating by study serves as a kind of “worst-case” scenario, whereas 10-fold cross-validation serves as a “best-case.” Attempting to classify active and inactive SLE patients from different data sets and different microarray platforms during cross-validation by study proved difficult, but module enrichment was able to smooth out much of the technical variation between data sets. 10-fold cross-validation simulated a more standardized diagnostic test. Although the data was sourced from three different microarray platforms, each cohort in the test set had many similar patients in the training set to facilitate classification by gene expression. If such a test may be reliably free from technical noise, it is likely that raw gene expression may perform very well.

RNA-Seq platforms, which produce transcript counts rather than probe intensity values, may display less technical variation across data sets because they are not dependent on the binding characteristics of pre-defined probes that differ among arrays. On the other hand, comparison of RNA-Seq and microarray samples may show that the two methods may deliver highly consistent results, so a microarray-based test may be feasible if it were only conducted on one platform. Constructing an optimal panel of genes similar to that identified by the random forest classifier may result in a simple, focused test to determine disease activity by gene expression data alone. Interestingly, module enrichment scores, which show little variation across platforms, may be used to develop diagnostic tests that leverage existing data sets, even if they are sourced from different platforms.

The strong performance of the random forest classifier indicates that nonlinear, decision tree-based methods of classification may be well suited to SLE diagnostics. This may be because decision trees ask questions about new samples sequentially and adaptively in contrast to other methods that approach variables from new samples all at once. Random forest is able to “understand” to an extent that different types of patients exist and that a one-size-fits-all approach may tend to misclassify those patients whose expression patterns make them a minority within their phenotype. To put it more simply, active patients that do not resemble the majority of active patients still have a strong chance of being properly classified by random forest.

The random forest classifier was used to assess the importance of each gene and module in patient classification. The most important genes were involved in a number of functions other than interferon signaling, such RNA processing, ubiquitylation, and mitochondrial processes. These pathways may play important roles in directing, or at least be indicative of, SLE disease activity. CD4 T cells originally contributed the most important modules, but when the modules were de-duplicated, CD14 monocyte-derived modules gained importance. This suggests that unique genes expressed by CD14 monocytes in tandem with interferon genes may prove to be informative in the study of cell-specific methods of SLE pathogenesis. Futhermore, it is important to note that modules that were negatively associated with disease activity were just as important in classification as positively associated modules. Study of underrepresented categories of transcripts may enhance an understanding of SLE activity.

While creating dedicated training and test sets may be preferable to cross-validation, this approach may require a large number of samples. Although there are large numbers of publicly available gene expression profiles of SLE patients, many of these profiles are not annotated with SLEDAI data. Furthermore, some data sets which include SLEDAI data show heavy class imbalance, which impedes classification. Cross-platform expression data may be integrated toward expanding the ability to classify active and inactive SLE patients.

The machine learning models developed provide the basis of personalized medicine for SLE patients. Integration of these approaches with high-throughput patient sampling technologies may unlock the potential to develop a simple blood test to predict SLE disease activity. These approaches may also be generalized to predict other SLE manifestations, such as organ involvement. A better understanding of the cellular processes that drive SLE pathogenesis may eventually lead to customized therapeutic strategies based on patients' unique patterns of cellular activation.

Gene expression data may be compiled from SLE patients as follows. Publicly available gene expression data and corresponding phenotypic data were mined from the Gene Expression Omnibus. Raw data sources for purified cell populations are as follows: GSE10325 (CD4: 8 SLE, 9 HC; CD19: 10 SLE, 8 HC; CD33: 9 SLE, 9 HC); GSE26975 (10 SLE LDG, 10 SLE Neutrophil, 9 HC Neutrophil); GSE38351 (CD14: 8 SLE, 12 HC). Raw data sources for SLE whole blood gene expression are as follows: GSE39088 (24 active, 13 inactive); GSE45291 (35 active, 257 inactive); GSE49454 (23 active, 26 inactive). 35 randomly sampled inactive patients were taken from GSE45291 to avoid a major imbalance between active and inactive SLE patients. Active SLE was defined as having an SLE Disease Activity Index (SLEDAI) of 6 or greater.

Quality control and normalization of raw data files may be performed as follows. Statistical analysis was conducted using R and relevant Bioconductor packages. Non-normalized arrays were inspected for visual artifacts or poor hybridization using Affy QC plots. PCA plots were used to inspect the raw data files for outliers. Data sets culled of outliers were cleaned of background noise and normalized using RMA, GCRMA, or NEQC where appropriate. Data sets were then filtered to remove probes with low intensity values and probes without gene annotation data. WB gene expression data sets were filtered to only include genes that passed quality control in all data sets. At this juncture, differential expression (DE) analysis and Weighted Gene Co-expression Network Analysis (WGCNA) were carried out on data sets. WB gene expression data sets were then further processed before machine learning analysis. WB gene expression values were centered and scaled to have zero-mean and unit-variance within each data set, and the standardized expression values from each data set were joined for classification.

Differential Expression analysis may be performed as follows. Normalized expression values were variance corrected using local empirical Bayesian shrinkage, and DE was assessed using the LIMMA R package. Resulting p-values were adjusted for multiple hypothesis testing using the Benjamini-Hochberg correction, which resulted in a false discovery rate (FDR). Significant genes within each study were filtered to retain DE genes with an FDR<0.2, which were considered statistically significant. The FDR was selected a priori to diminish the number of genes that may be excluded as false negatives. Rank-rank hypergeometric overlap between data sets was assessed using the RRHO R package. Additional analyses examined differentially expressed genes with an FDR<0.05.

Weighted Gene Co-expression Network Analysis (WGCNA) of purified cell populations may be performed as follows. Log 2-normalized microarray expression values from purified CD4, CD14, CD19, CD33, and low density granulocyte (LDG) populations were used as input to WGCNA to conduct an unsupervised clustering analysis, resulting in co-expression “modules,” or groups of densely interconnected genes which may correspond to comparably regulated biologic pathways. For each experiment, an approximately scale-free topology matrix (TOM) was first calculated to encode the network strength between probes. Probes were clustered into WGCNA modules based on TOM distances. Resultant dendrograms of correlation networks were trimmed to isolate individual modular groups of probes by partitioning around medoids and labeled using color assignments based on module size. Expression profiles of genes within modules were summarized by a module eigengene (ME), which is analogous to the module's first principal component. MEs act as characteristic expression values for their respective modules and may be correlated with sample traits such as SLEDAI or cell type. This was done by Pearson correlation for continuous or semi-continuous traits and by point-biserial correlation for dichotomous traits.

WGCNA modules from CD4, CD14, CD19, and CD33 cells were tested for correlation to SLEDAI. SLEDAI information was not available for the LDG modules, so the two modules provided are descriptive of LDGs compared to SLE neutrophils and HC neutrophils.

Plasma cell modules were generated by differential expression analysis and not WGCNA, but were included because of the established importance of plasma cells in SLE pathogenesis and their increase in active disease.

Gene Set Variation Analysis (GSVA)-based enrichment of expression data may be performed as follows. The GSVA R package was used as a non-parametric method for estimating the variation of pre-defined gene sets in SLE WB gene expression data sets. Standardized expression values from WB data sets were used to test for enrichment of cell-specific WGCNA gene modules using the Single-sample Gene Set Enrichment Analysis (ssGSEA) method, which scores single samples in isolation and is thus shielded from technical variation within and among data sets. Statistical analysis of GSVA enrichment scores was done by Spearman correlation or Welch's unequal variances t-test, where appropriate. Effect sizes were assessed by Cohen's d.

Machine learning algorithms and parameters may be developed as follows. Three distinct machine learning algorithms were employed to test biased and unbiased approaches to microarray data analysis. The biased approach involved GSVA enrichment of disease-associated, cell-specific modules, and the unbiased approach employed all available gene expression data in the WB. An elastic generalized linear model (GLM), k-nearest neighbors classifier (KNN), and random forest (RF) classifier were deployed to classify active and inactive SLE patients and determine whether gene expression may serve as a general predictor of disease activity. GLM, KNN, and RF were deployed using the glmnet, caret, and randomForest R packages, respectively.

GLM carries out logistic regression with a tunable elastic penalty term to find a balance between the L1 (lasso) and L2 (ridge) penalties and thereby facilitate variable selection. For our predictions, the elastic penalty was set to 0.9, specifying a penalty that is 90% lasso and 10% ridge in order to generate sparse solutions. KNN classifies unknown samples based on their proximity to a set number k of known samples. K was set to 5% of the size of the training set. If the initial value of k was even, 1 was added in order to avoid ties. RF generates 500 decision trees which vote on the class of each sample. The Gini impurity index, a measure of misclassification error, was used to evaluate the importance of variables. In addition to these three approaches, pooled predictions were assigned based on the average class probabilities across the three classifiers.

Validation approaches may be performed as follows. The performance of each machine learning algorithm was evaluated by 2 different forms of cross-validation. First, a random 10-fold cross-validation was carried out by randomly assigning each patient to one of 10 groups. For each pass of cross-validation, one group was held out as a test set, and the classifiers were trained on the remaining data. Next, as the data came from three separate studies, study-based cross-validation was also done to determine the effects of systematic technical differences among data sets on classification performance. In this circumstance, the classifiers were trained on one data set and tested in the other two data sets. Accuracy was assessed as the proportion of patients correctly classified across all testing folds. Performance metrics such as sensitivity and specificity were assessed after cross-validation by agglomerating class probabilities and assignments from each fold or study. Receiver Operating Characteristic (ROC) curves were generated using the pROC R package.

### Example 3: Molecular Endotyping Analysis for Identifying Subsets of Patients with Systemic Lupus Erythematosus Who are Candidates to be Enrolled in Clinical Trials and have a Propensity to Respond to Specific Drugs

Using methods and systems of the present disclosure, molecular endotyping analysis may be performed for identifying subsets of patients with Systemic Lupus Erythematosus who are candidates to be enrolled in clinical trials and have a propensity to respond to specific drugs. In precision medicine, identifying patients who may be appropriate candidates for entry into a clinical trial and/or who have a propensity to respond to a specific therapy is crucial, for example, to de-risk clinical trials. In trials of complex diseases, such as Systemic Lupus Erythematosus (SLE), with current approaches, it may be difficult to identify significant phenotypic and transcriptomic differences between subjects who may be responders and non-responders to specific therapies. For example, post-hoc analysis of the ILLUMINATE trials of tabalumab in SLE by Lilly was unable to identify any genes that were differentially expressed between responders and non-responders.

A hypothesis may be that SLE in particular is a common clinical manifestation of several molecular abnormalities or endotypes, each driven by a distinct combination of cell types and immune or inflammatory mechanisms. Incorporating knowledge of endotypes of individual subjects (e.g., SLE patients) may be a crucial step in the identification of subjects appropriate to enter a clinical trial and/or to benefit from a specific therapy (e.g., targeted therapy to treat SLE).

Methods and systems of the present disclosure can be used to determine whether distinct phenotypic and/or transcriptomic subsets of subjects exist and, subsequently, whether each group is likely to respond to specific therapies. The appropriate or inappropriate entry of such patients into trials may inflate or deflate the efficacy of a clinically tested treatment. Moreover, an investigational product that fails in a clinical trial may later be documented to be highly efficacious when tested on a patient subset with an appropriate molecular endotype.

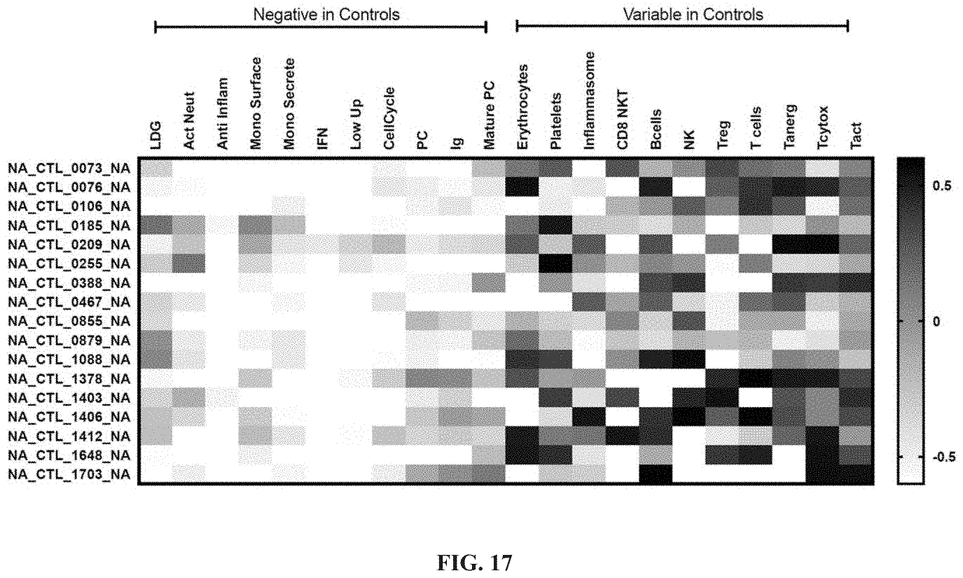

The ability to stratify SLE patients into different groups associated with different types of disease or disease activity by transcriptomic signatures provides significant advantages toward determining appropriate patient care and enrollment in clinical trials. Using methods and systems disclosed herein, immunologically active SLE patients can be distinguished for entry into SLE clinical trials or to change patients to a more appropriate drug regimen. Results demonstrated that SLE patients can be grouped (e.g., clustered or distinguished) by their transcriptomic signatures. For example, FIG. 17 shows a heat map showing the variation of gene expression in normal controls. Differentially expressed (DE) transcripts pertaining to cell type and process signatures in 10 SLE whole blood and peripheral blood mononuclear cell microarray datasets were used to create modules of genes potentially enriched in SLE patients determined by Gene Set Variation Analysis (GSVA). Although significant differences in transcripts pertaining to B cells, T cells, erythrocytes, and platelets between SLE patients may be observed in SLE, it is notable that at the level of RNA transcription, these signatures may not be uniformly expressed in the healthy controls (HC) (FIG. 17) from several SLE datasets, demonstrating that the differences in these signatures are related to heterogeneity in controls unrelated to SLE.

A suite of clustering techniques may be used to partition clinical trial enrollees at baseline based on gene expression data and/or clinical parameters. These methods may be used to drastically reduce the dimensionality of transcriptomic-scale data, even for cases in which Principal Component Analysis (PCA) fails to generate an informative set of variables.

Furthermore, extensive analysis of the contribution of subject demographic and clinical variables revealed that many of the differences between datasets and patients were not related to the disease, but to the patient's ancestry, gender, or the subject's drug regimen, each of which may independently influence the transcriptomic signature. Thus, in order to determine whether there were different types of SLE molecular endotypes common amongst patients of different ancestral backgrounds, different SLE standard of care treatments and different manifestations, 11 transcriptomic signatures negative in controls were used for principal component analysis (PCA) of 1,566 female SLE patients divided into three ancestry sub-groups; African ancestry (AA, n=216), European ancestry (EA, n=1,118) and Native Southern American ancestry (NAA, n=232). An 11-dimension principal component analysis (PCA) was performed, and results established that principal component 1 (PC1) was determined by whether the patient had circulating plasma cells (PC1−) or myeloid cells (PC1+); in other words, the greatest separation between patients was affected by whether they had a plasma cell or Myeloid Cell dominated transcriptomic signature. As another example, PC2 was roughly half the contribution of PC1 and was related to the difference between the presence of a low-density granulocyte (LDG)/neutrophil signature and the interferon (IFN) signature. As shown in FIG. 17, heatmap clustering of the PCA analysis demonstrated two prominent divisions between the 11 immunologically related modules in the SLE patients. Plasma cell, Immunoglobulins, Mature PC, and cell cycle grouped together (FIG. 17, left) and all the other signatures grouped together including IFN and anti-inflammation. PCA and heatmap divisions were the same between ancestries, except that more AA SLE patients were PC1− (plasma cells) than PC1+(myeloid) and more NAA SLE patients were PC1+(myeloid) than PC1− (plasma cell).

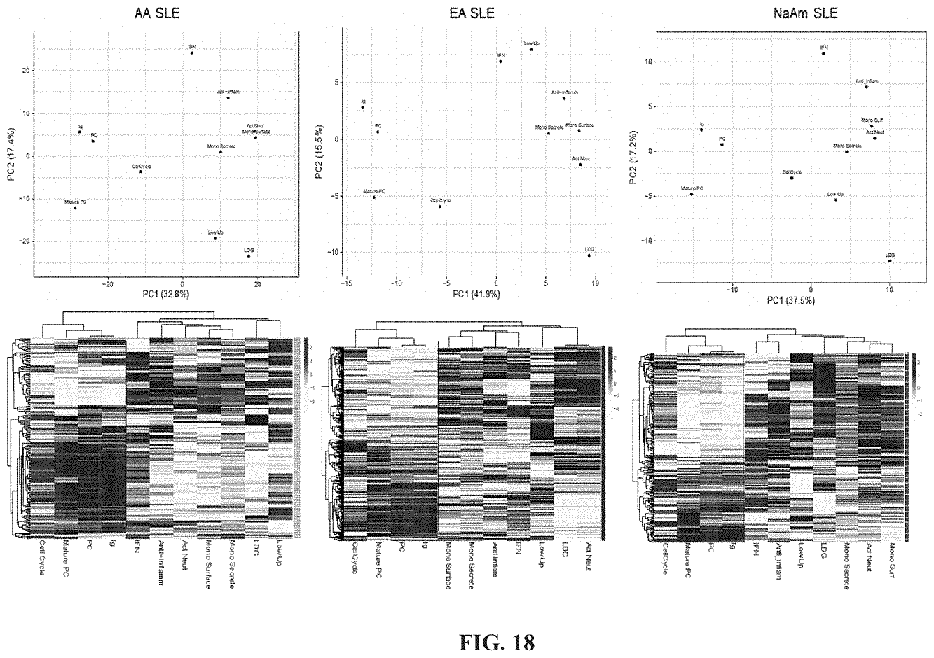

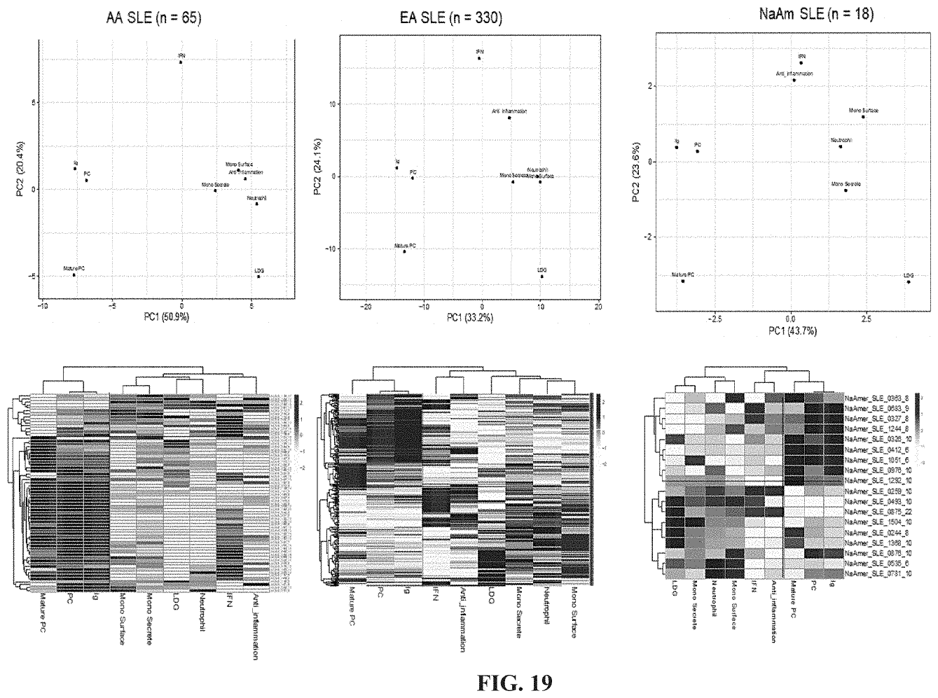

FIG. 18 shows PCA and heatmap clustering of AA, EA, and NAA SLE patients for 11 GSVA enrichment modules negative in healthy controls (HC). GSVA enrichment scores were uploaded to ClustVis, and PCA plots were generated. Low Up, a signature derived from SLE patients with no enrichment for IFN, PC, or myeloid cells (FCGR1A, SNORD80, SNORD44, SNORD47, SNORD24, CEACAM1, and LGALS1) changed where it grouped depending on ancestry. Heatmaps were generated using correlation clustering distance for both rows and columns. The heatmap clustering of the 11 modules revealed a dichotomy in SLE patient transcriptomic signatures; SLE patients with strong PC signatures were less likely to have strong myeloid signatures, especially in patients of AA ancestry, and in SLE patients with strong myeloid signatures, there were fewer contributing plasma cell signatures. Interferon signatures occurred with either myeloid or plasma cell signatures but were more often paired with strong monocyte signatures. Low density granulocytes/neutrophils were associated with the myeloid signature as well. Importantly, within each ancestral background, there were both plasma cell and myeloid SLE patients (FIG. 18). Steroids may be shown to be associated with low-density granulocyte enrichment and low-density granulocytes were important in both PC1 as part of the myeloid signature and the signature dominated PC2; therefore, PCA plots and heatmaps were generated for SLE patients not taking steroids. AA SLE patients not taking steroids had few patients with myeloid SLE signatures. The proportion of EA and NAA SLE patients with myeloid signatures decreased, although since most NAA SLE patients were on steroids there were very few patients in this analysis (FIG. 19).

FIG. 19 shows PCA and heatmap clustering of AA, EA, and NAA SLE Patients not taking steroids for 9 GSVA enrichment modules negative in healthy controls (HC). The cell cycle and Low Up modules were removed, GSVA enrichment scores for the 9 remaining modules were uploaded to ClustVis, and PCA plots and heatmaps were generated. Heatmaps were generated using correlation clustering distance for both rows and columns.

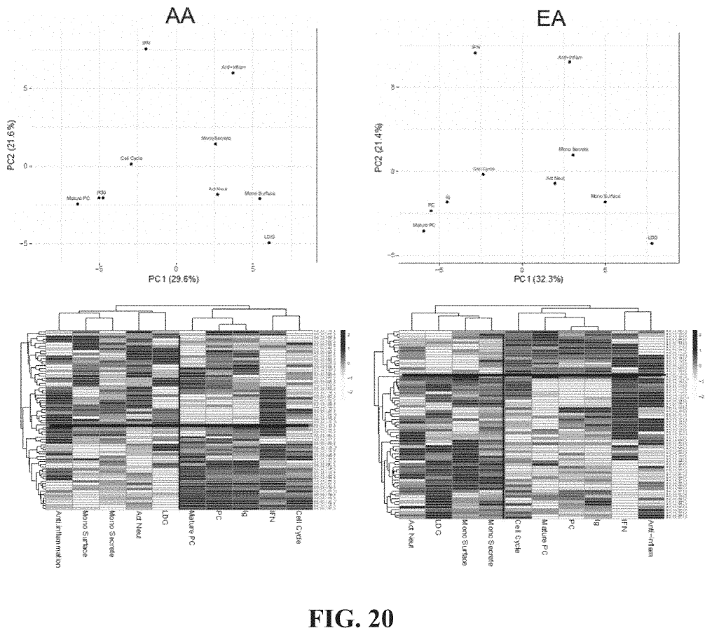

SLE microarray datasets have wide heterogeneity related to the disease but also because of the different platforms to measure transcripts and variability; therefore, it was important to establish that the divisions found in the 1,566 female illuminate patients (GSE88884) are applicable to SLE patients assayed on a different array platform. AA and EA SLE patients with low disease activity (SLEDAI range 2-11) from dataset GSE45291 had PC1 and PC2 components similar to GSE88884 patients and demonstrated the same dichotomy in having either a plasma cell or Myeloid cell type of SLE. As was shown for dataset GSE88884, there were a higher percentage of SLE patients with AA ancestry and plasma cell SLE, and a higher percentage of SLE patients with EA ancestry and myeloid SLE (FIG. 20).

FIG. 20 shows PCA and heatmap clustering of a second, independent microarray dataset demonstrate that SLE patients divided into plasma cell or myeloid lupus. 73 AA and 71 EA patients from GSE45291 with SLEDAI in the range of 2-11 had GSVA scores calculated for 10 signatures. ClustVis was used to determine PC1 and PC2 for AA (top left) and EA (top right). Heatmaps show the patient distribution for the plasma cell related GSVA enrichment categories (Cell cycle, Mature plasma cell, plasma cell, and immunoglobulin chains) versus the myeloid cell enrichment categories (Interferon, Anti-Inflammation, Mono Surface, Mono Secrete, LDG, and Act Neut). Dataset GSE45291 was assayed on Affymetrix chip HT HG-U133+ PM which does not have probes for small nucleolar RNAs that make up most of the Low Up signature.

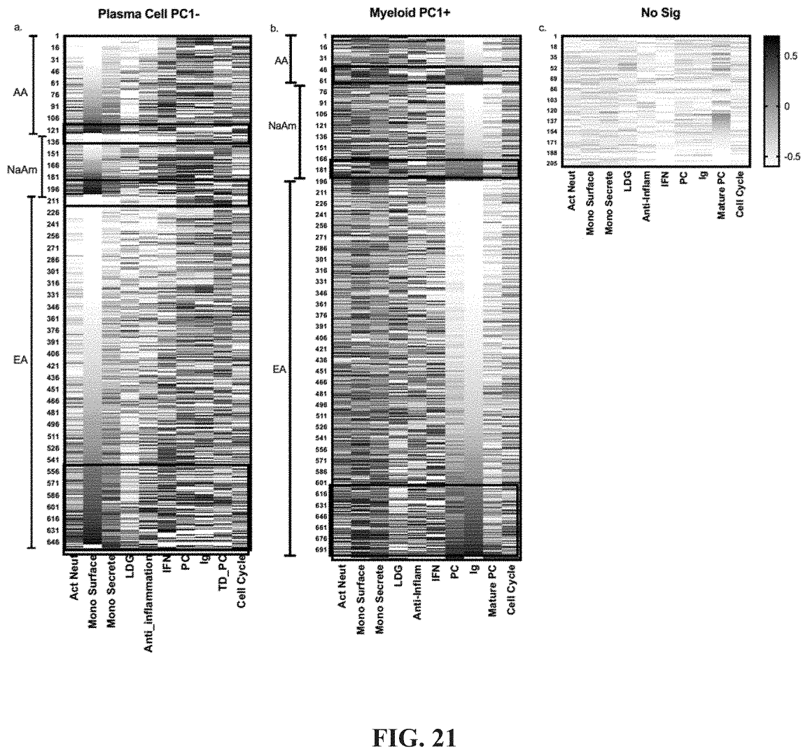

209 female SLE patients (13.3%) enrolled in the Illuminate clinical trial (GSE88884) had GSVA scores for the 10 immunologically related modules indistinguishable from HC (not including LowUp, which was based on patients which were difficult to distinguish from HC). These immunologically inactive SLE patients represented all three ancestry sets studied: 161 EA (14.4%), 25 AA (11.6%), and 23 NAA (10.3%); they were categorized as having no immunologically related signature (No Sig). PCA analysis was performed using the 10 immunologically related GSVA modules, and the PC1 loadings for each patient were used to determine the classification of either plasma cell or myleoid SLE based on whether they were PC1− (enriched for modules for plasma cell, Ig) or PC1+(enriched for myeloid modules) (FIG. 21).

FIG. 21 shows heatmap clustering of SLE patients by enrichment of 10 immunologically related modules. SLE patients were grouped on the basis of having a negative PC1 loading score (plasma cell, left), a positive PC1 loading score (myeloid, middle), no enrichment of the 10 modules (No Sig, right). SLE patients within Plasma Cell or Myeloid that also expressed the opposite signature, as defined by either having a Mono GSVA enrichment score of at least 0.1, are identified by black boxes.

SLE disease measures were compared for each ancestry between PC1−, PC1+, and No Sig SLE patients. Although the average SLEDAI was generally higher for SLE patients expressing either PC or Myeloid modules compared to the No Sig group of patients, there was not a discernable cut-off for a SLEDAI which was suitable for defining a patient with no transcriptional sign of immunological perturbation. The mean SLEDAI was significantly higher (p<0.05 by Tukey's multiple comparisons test) for myeloid among AA patients, plasma cell and myeloid among EA patients, and plasma cell for NAA patients, as compared to the No Sig category within each ancestry. No significant difference in SLEDAI was found between SLE patients with myeloid versus plasma cell SLE. Steroid usage was significantly higher (p<0.05) for the myeloid signature for all three ancestries (Table 12).

TABLE 12
Disease differences between PC1−, PC1+, and No Sig categories

| | AA (n = 216) | | | | | | EA (n = 1118) | | | | | | NaAm (n = 232) | | | | | |
|---|---|---|---|---|---|---|---|---|---|---|---|---|---|---|---|---|---|---|
| | PC1− | | PC1+ | | No Sig | | PC1− | | PC1+ | | No Sig | | PC1− | | PC1+ | | No Sig | |
| n | 125 | | 66 | | 25 | | 449 | | 508 | | 161 | | 80 | | 129 | | 23 | |
| average | 10.73 | | 10.97\hat{ } | | 8.8 | | 10.74^{#} | | 10.21^{##} | | 9.35 | | 11.66* | | 11.124 | | 9.04 | |
| SLEDAI | | | | | | | | | | | | | | | | | | |
| median | 10 | | 10 | | 8 | | 10 | | 10 | | 8 | | 11 | | 10 | | 8 | |
| SLEDAI | | | | | | | | | | | | | | | | | | |
| mode | 8 | | 8 | | 8 | | 10 | | 8 | | 8 | | 12 | | 10 | | 8 | |
| SLEDAI | | | | | | | | | | | | | | | | | | |
| # | 3.6 | | 3.8 | | 3.2 | | 3.8 | | 3.6 | | 3.2 | | 4 | | 4 | | 3.5 | |
| Manifest | | | | | | | | | | | | | | | | | | |
| average | 7.99 | | 9.83\hat{ }\hat{ } | | 4.2 | | 9.05^{$} | | 9.47^{$$} | | 4.13 | | 10.76 | | 12.98** | | 6.52 | |
| steroid | | | | | | | | | | | | | | | | | | |
| median | 5 | | 10 | | 0 | | 7.5 | | 10 | | 0 | | 10 | | 10 | | 5 | |
| steroid | | | | | | | | | | | | | | | | | | |
| mode | 0 | | 10 | | 0 | | 0 | | 10 | | 0 | | 10 | | 10 | | 5 | |
| steroid | | | | | | | | | | | | | | | | | | |
| MMF or | 16.8% | (21) | 41% | (27) | 16% | (4) | 12.2% | (55) | 22% | (113) | 19% | (31) | 24% | (19) | 36% | (47) | 22% | (5) |
| MTX (n) | | | | | | | | | | | | | | | | | | |
| dsDNA | 40% | (50) | 32% | (21) | 20% | (5) | 22% | (98) | 26% | (133) | 16% | (25) | 23% | (18) | 30% | (39) | 17% | (4) |
| (n) | | | | | | | | | | | | | | | | | | |
| lowC | 3% | (4) | 11% | (7) | 0% | | 8% | (37) | 7% | (38) | 11% | (18) | 8% | (6) | 8% | (10) | 4% | (1) |
| (n) | | | | | | | | | | | | | | | | | | |
| dsDAN + | 27% | (34) | 24% | (16) | 8% | (2) | 45% | (200) | 30% | (152) | 7% | (12) | 51% | (41) | 28% | (46) | 13% | (3) |
| lowC (n) | | | | | | | | | | | | | | | | | | |

\hat{ }AA SLEDAI PC1+ to No Sig p = .05
\hat{ }\hat{ }AA SLEDAI PC1+ Steroid to No Sig p = .02
ANOVA & Tukey's Multiple Comparison
^{#}EA SLEDAI PC1− to No Sig p = .0001
^{##}EA SLEDAI PC1+ to No Sig p = .03
^{$}EA Steroid PC1− to No Sig p < .0001
^{$$}EA Steroid PC1+ to No Sig p < .0001
*NaAm SLEDAI PC1− to No Sig p = .02
**NaAM Steroid PC1+ to No Sig = .001

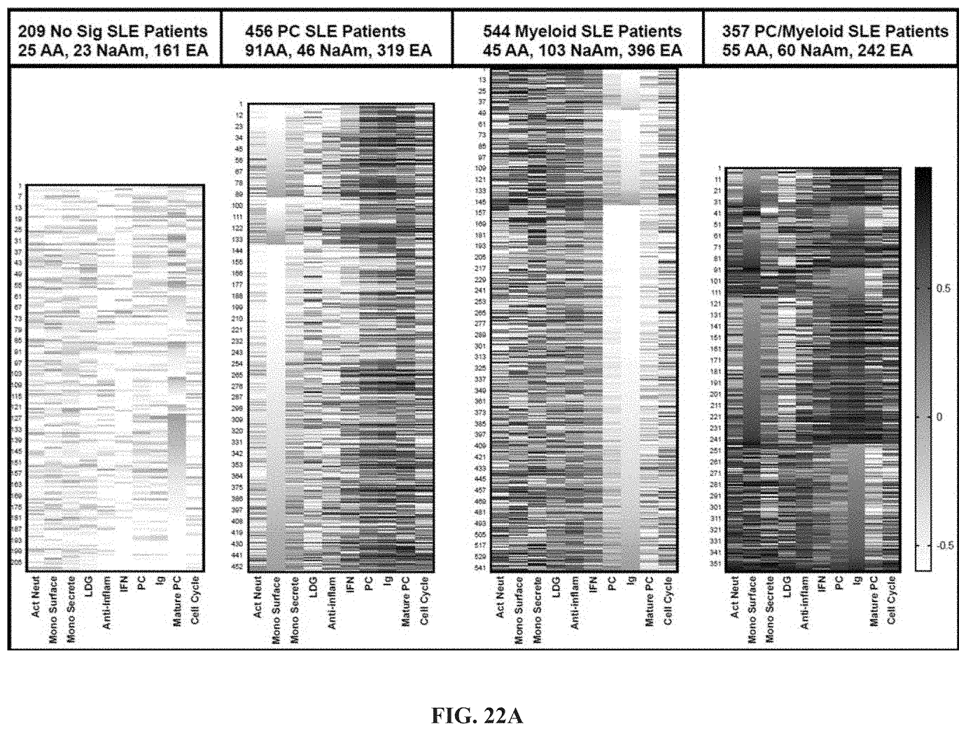

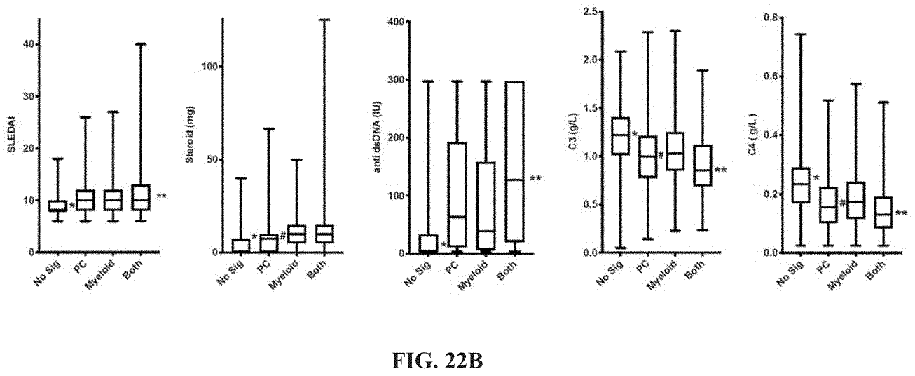

A heatmap visualization of the different ancestral SLE patients together as plasma cell, myeloid, or No Sig was generated; it revealed SLE patients with both plasma cell and myeloid signatures. Patients with both signatures (as determined by having a GSVA enrichment score 2 standard deviations above healthy control GSVA scores for both the myeloid and the plasma cell signatures) were combined to form a new group, “Both” (FIGS. 22A-22B).

FIGS. 22A-22B show heatmap clustering of SLE patients by enrichment of 10 immunologically related modules. Four divisions were found for the 1,566 female SLE patients enrolled in the ILL clinical trials. Based on PC1 loadings for PCA of patients, PC and myeloid SLE patients were sorted by the opposite GSVA enrichment signature: monocyte cell surface for the PC signature (PCA PC1−) and Ig for the myeloid signature (PCA PC1+), and SLE patients with GSVA enrichment scores of at least 0.1 for the opposite signature were removed and reclassified as having both signatures (FIG. 22A). SLE patients of all ancestries were grouped based on the four classifications. ANOVA and Tukey's multiple comparisons test was performed between the four groupings (FIG. 22B). For SLEDAI, No sig* was significantly lower from PC, Myeloid, and Both (p<0.05), and Both** was significantly (p<0.05) higher than PC and Myeloid. For steroid usage, No sig* was significantly lower (p<0.0001) than all other groups. PC was significantly lower than Both (p=0.0053). For aDS DNA, No sig* was significantly lower (p<0.0001) than all other groups and Both** was significantly higher (p<0.0001) than all other groups. For complement C3 and C4, all groups were significantly different (p<0.01) from each other; No sig* had the highest values, followed by myeloid. PC had lower values than No Sig and Myeloid, but Both** had the lowest C3 and C4 values.

Heatmap clustering of the four groups demonstrated that similar percentages of AA, EA, and NAA patients were found in the No Sig (AA 12%, NAA 12%, EA 13%) and Both (AA 25%, NAA 26%, EA 22%) groups, but there were a higher percentage of AA patients in the plasma cell only (p<0.05, Fisher's Exact Test; AA 42%, NAA 20%, EA 29%) and NAA in myeloid only (p<0.05 Fisher's Exact Test; AA 21%, NaAm 44%, EA 35%) (FIG. 22A). Comparison of the SLEDAI, steroid dose, anti-double stranded DNA levels, C3, and C4 serum measurements by ANOVA revealed significant differences between the groups. The No Sig classification with no immunologic transcriptomic signatures had the lowest SLEDAI and anti-double stranded DNA levels, and the highest C3 and C4 levels. Interestingly, this group was also taking the least amount of corticosteroids. SLE patients with both a myeloid and a plasma cell transcriptomic signature had the highest SLEDAI and highest percentage of anti-double stranded DNA values, and the lowest C3 and C4 values. This group was taking similar steroids to the myeloid only group and significantly more steroids than the No Sig or plasma cell only group. The plasma cell only and myeloid only groups were similar for SLEDAI and anti-double stranded DNA levels, but the plasma cell group had significantly lower C3 and C4 levels and were taking less steroids (FIG. 22B).

The Low Up Category was derived from the highest overexpressed transcripts by log fold change (FDR<0.05) between patients not separated from healthy control after initial PCA analysis of all the GSE88884 dataset log 2 expression values. This signature was expressed in 30% of the No Sig SLE patients and was increased in more immunologically transcriptomic patients: plasma cell only, 39% (180/456); myeloid only, 55% (298/544); and Both, 71% (254/357).

### Example 4: Molecular Endotyping Analysis for Identifying Subsets of Patients with Systemic Lupus Erythematosus Who are Candidates to be Enrolled in Clinical Trials and have a Propensity to Respond to Specific Drugs

Using methods and systems of the present disclosure, molecular endotyping analysis may be performed for identifying subsets of patients with Systemic Lupus Erythematosus who are candidates to be enrolled in clinical trials and have a propensity to respond to specific drugs.

Weighted gene co-expression network analysis (WGCNA) was performed, using a computer program in R that takes a microarray or RNAseq dataset and identifies modules (groups) of genes that are co-expressed in a similar manner in the samples and or controls. Each individual sample is designated with a positive or negative value for each module indicating whether the individual sample co-expresses the genes in the module or does not. The number of groups or modules WGCNA identifies is unbiased in that there is no preconceived number of modules in a data set. The gene expression value of a module (eigengene) is used to determine whether an individual patient expresses a module or modules, whether groups of patients can be identified who express a similar constellation of modules and, also, whether there are patterns to the groupings. This approach can also be employed to determine whether positivity of specific WGCNA modules is correlated to SLE disease measures, such as disease activity, autoantibodies, and complement abnormalities. and other confounding factors such as patient ancestry.

WGCNA was performed on a set of 810 female systemic lupus erythematosus (SLE) patients and 11 healthy control whole blood samples. Patients were mainly of European ancestry (EA), African ancestry (AA), or Southern Native American ancestry (NAA; Guatemala, Peru, Ecuador) ancestry. The WGCNA results identified 13 discrete modules. Characterization of the modules was performed using multiple programs, such as CellScan and I-scope to determine whether a module was enriched in cellular markers corresponding to a specific cell type, and BIG-C to determine whether modules were enriched in specific cellular function or process. This analysis revealed prominent signatures related to cell types and processes, IFN signaling, and MicroRNA in 12 of the 13 modules. One module, turquoise (modules are randomly designated with colors for convenience), had more than 5,000 genes and no discernable cell type or function. This module also had the lowest percentage of genes that were differentially expressed between SLE patients and controls in separate limma analysis (for example, AA to CTL only had 1.67% of the turquoise genes differentially expressed (DE) compared to CTL).

Table 13 shows WGCNA modules identified in SLE patients.

TABLE 13
WGCNA modules identified in SLE patients

| | | Percent Positive of DE transcripts in Module | | | | | | | | | | | | |
|---|---|---|---|---|---|---|---|---|---|---|---|---|---|---|
| | | | | | | | | | | | | | Granulo- | |
| | | | | | IL1, | un- | | | | | | | cytes/ | |
| | | | PC | Lympho- | Inflamm | known | T cell, | Plate- | Erythro- | Micro | Myeloid | NKTR | Basophils | CD14+ |
| number of genes | | IFN | ma- | cytes | myeloid | tur- | SNORAs | lets | cytes | RNA | light | IL16 | midnight | TGFB1+ |
| DE to control | | black | genta | blue | brown | quoise | pink | purple | green | cyan | cyan | red | blue | yellow |
| AA | 1591 | 70.03 | 15.58 | 16.32 | 10.13 | 1.67 | 6.55 | 14.04 | 6.95 | 2.80 | 4.71 | 7.79 | 7.49 | 3.56 |
| to | | | | | | | | | | | | | | |
| ctl | | | | | | | | | | | | | | |
| EA | 1906 | 71.18 | 6.49 | 18.25 | 25.11 | 2.62 | 17.86 | 3.51 | 4.63 | 0.93 | 7.58 | 15.15 | 10.08 | 3.21 |
| to | | | | | | | | | | | | | | |
| ctl | | | | | | | | | | | | | | |
| NaAm | 6580 | 85.59 | 20.35 | 74.38 | 64.76 | 9.82 | 32.14 | 23.98 | 26.77 | 37.58 | 25.42 | 45.45 | 42.64 | 25.19 |
| to | | | | | | | | | | | | | | |
| ctl | | | | | | | | | | | | | | |

Modules with negative eigengene values in healthy human controls were the IFN PRR module (black), plasma cell module (magenta), inflammatory myeloid module (brown), MicroRNA module (cyan) and platelet module (purple). Modules with positive expression in healthy controls were NKTR (red), lymphocytes (blue) and T cells (pink) (Table 14).

TABLE 14
WGCNA modules and their eigengene values in healthy controls

| | | | | | | Modules with variable expression in Controls | |
|---|---|---|---|---|---|---|---|
| | Decreased in Controls | | | | | Myeloid, SELL, | |
| | | | Inflammatory | | | TBK1, CD16, SYK, | Basophils - VEGFA, |
| | | | Myeloid Cells, IL1, | | | TANK, IRAK4, AOAH, | METRNL, OSM, LCAT, |
| | IFN | Plasma | Tons of Secreted | MicroRNA | Platelets | not activated | LTBR, LILRB5 LCE1F, |
| | black | Cells magenta | Protein Genes brown | TNFSF4 cyan | purple | light cyan | S1PR4 mignight blue |
| CTL.0073.NA | −0.06 | −0.03 | −0.07 | −0.03 | −0.01 | −0.06 | 0.00 |
| CTL.0106.NA | −0.04 | −0.02 | −0.04 | −0.02 | −0.04 | −0.03 | 0.00 |
| CTL0256.NA | −0.05 | −0.01 | −0.04 | −0.01 | 0.00 | −0.04 | −0.01 |
| CTL.0343.NA | −0.04 | −0.04 | 0.02 | 0.04 | −0.02 | 0.05 | −0.03 |
| CTL.0388.NA | −0.05 | −0.02 | −0.03 | 0.00 | −0.01 | 0.01 | −0.03 |
| CTL.0581.NA | −0.06 | −0.03 | −0.03 | −0.01 | −0.01 | −0.04 | 0.00 |
| CTL.0812.NA | −0.05 | −0.03 | 0.00 | 0.01 | 0.04 | −0.01 | 0.01 |
| CTL.0879.NA | −0.06 | −0.02 | 0.00 | 0.00 | −0.02 | −0.02 | 0.02 |
| CTL.1403.NA | −0.03 | −0.02 | −0.02 | 0.00 | 0.00 | 0.00 | −0.02 |
| CTL.1406.NA | −0.04 | −0.01 | −0.01 | 0.03 | −0.01 | 0.02 | −0.03 |
| CTL.1703.NA | −0.04 | 0.00 | −0.03 | −0.02 | 0.00 | 0.00 | −0.05 |

| | Modules with variable expression in Controls | | | | | |
|---|---|---|---|---|---|---|
| | CD14 Monocytes, | | | | | |
| | ox phos and tea | | | | | |
| | cycle, peroxisomes, | | | | | |
| | proteasome, TGFB1, | Erythrocytes, | No discernable | Increased in Controls | | |
| | TNFSF8, IKLYZ, FCN2, | GYPAE, GYPAB, | cell type or | NKTR, IL16 | Lymphocytes, | T |
| | HBB, HAVCR2, CCR2+, | KEL, RHD, BSG | function module >5000 | T cell receptor | T cells, | cells |
| | MS4A6A, BTN3A3, yellow | green | turquoise | J chains red | B cells blue | pink |
| CTL.0073.NA | −0.03 | 0.03 | 0.04 | 0.02 | 0.01 | 0.05 |
| CTL.0106.NA | −0.02 | −0.05 | 0.02 | 0.00 | 0.02 | 0.03 |
| CTL0256.NA | −0.01 | 0.05 | 0.01 | 0.02 | 0.01 | 0.04 |
| CTL.0343.NA | 0.05 | 0.01 | −0.04 | 0.06 | 0.02 | 0.02 |
| CTL.0388.NA | 0.03 | −0.03 | −0.01 | 0.04 | 0.04 | 0.01 |
| CTL.0581.NA | −0.02 | 0.06 | 0.02 | 0.00 | 0.00 | 0.02 |
| CTL.0812.NA | −0.02 | 0.05 | 0.03 | −0.01 | −0.02 | 0.00 |
| CTL.0879.NA | −0.02 | 0.03 | 0.04 | −0.01 | 0.00 | 0.02 |
| CTL.1403.NA | 0.01 | −0.01 | −0.01 | 0.03 | 0.03 | 0.02 |
| CTL.1406.NA | 0.04 | −0.02 | −0.04 | 0.0 | 0.04 | 0.04 |
| CTL.1703.NA | 0.04 | −0.02 | −0.02 | 0.04 | 0.06 | 0.01 |

As shown in Table 15, WGCNA identified four modules with correlation to the presence of SLE: IFN signaling and pattern recognition receptors (black), plasma cells (magenta), inflammatory myeloid cells (brown) and T cells (pink). The IFN and plasma cell modules had a relationship to the lupus disease activity measure SLEDAI and also to anti-double stranded DNA antibodies (dsDNA) and a negative relationship to complement protein C3 and C4 levels, important serum components associated with active SLE disease. Inflammatory myeloid cells were significantly correlated to anti-double stranded DNA, but not to low complement or the SLEDAI. T cells (pink) had a negative correlation to the SLE cohort and a negative relationship to the presence of anti-double stranded DNA autoantibodies and a positive relationship to complement C3 and C4 levels.

TABLE 15
WGCNA module correlations in 810 female SLE patients

| | assigned color | module Count | Cohort | Cohort p | SLEDAI | SLEDAI. p | dsDNA IU | dsDNA IU.p | C3 GperL |
|---|---|---|---|---|---|---|---|---|---|
| IFN and | black | 347 | 0.16 | 3.6E−06 | 0.25 | 9.9E−13 | 0.30 | 4.9E−19 | −0.32 |
| PRR | | | | | | | | | |
| Plasma Cells | magenta | 231 | 0.07 | 0.05778 | 0.22 | 9.7E−11 | 0.29 | 1.8E−17 | −0.32 |
| Inflammatory | brown | 908 | 0.07 | 0.03332 | 0.05 | 0.18802 | 0.10 | 0.0054 | 0.00 |
| Myeloid | | | | | | | | | |
| Cells | | | | | | | | | |
| Micro RNA | cyan | 322 | 0.00 | 0.9426 | 0.04 | 0.20196 | 0.00 | 0.99021 | 0.10 |
| Platelets | purple | 171 | 0.02 | 0.50223 | −0.03 | 0.33369 | 0.02 | 0.48014 | 0.20 |
| Myeloid. Not | lightcyan | 594 | 0.04 | 0.3008 | 0.05 | 0.16035 | 0.10 | 0.00622 | −0.05 |
| activated. | | | | | | | | | |
| Basophils | midnightblue | 387 | 0.04 | 0.28478 | 0.03 | 0.43885 | −0.02 | 0.59274 | 0.10 |
| T cells | pink | 336 | −0.08 | 0.01916 | −0.04 | 0.20566 | −0.16 | 5.1E−06 | 0.18 |
| Lymphocytes | blue | 3365 | −0.06 | 0.06677 | −0.03 | 0.39424 | −0.03 | 0.40269 | −0.08 |
| T and B | | | | | | | | | |
| cells, mRNA | | | | | | | | | |
| translation | | | | | | | | | |
| NKTR, IL16 | red | 462 | −0.07 | 0.05007 | 0.01 | 0.87992 | −0.06 | 0.09848 | 0.05 |
| Unknown | turquoise | 5569 | −0.01 | 0.74594 | −0.04 | 0.23356 | −0.07 | 0.03969 | 0.09 |
| Monocyte | yellow | 1433 | −0.01 | 0.68829 | 0.05 | 0.19486 | 0.07 | 0.05707 | −0.12 |
| TGFB1 | | | | | | | | | |
| CCR2+ | | | | | | | | | |
| Erythrocytes | green | 691 | −0.03 | 0.4157 | −0.06 | 0.10228 | −0.11 | 0.00246 | 0.21 |

| | C3 | | C4 | | | | Race | | |
|---|---|---|---|---|---|---|---|---|---|
| | GperL.p | C4 GperL | GperL.p | Race AA | Race AA.p | Race NaAm | NaAm.p | Race EA | Race EA.p |
| IFN and | 2.5E−21 | −0.28 | 1.2E−16 | 0.04 | 0.2396 | 0.08 | 0.03115 | −0.08 | 0.02481 |
| PRR | | | | | | | | | |
| Plasma Cells | 1.4E−21 | −0.30 | 2E−18 | 0.12 | 0.00088 | −0.06 | 0.07102 | −0.06 | 0.10054 |
| Inflammatory | 0.91912 | −0.01 | 0.80436 | −0.11 | 0.00162 | 0.11 | 0.00202 | 0.02 | 0.4957 |
| Myeloid | | | | | | | | | |
| Cells | | | | | | | | | |
| Micro RNA | 0.00379 | 0.09 | 0.01468 | 0.00 | 0.96314 | 0.13 | 0.00022 | −0.08 | 0.02075 |
| Platelets | 3.3E−09 | 0.16 | 2.8E−06 | 0.10 | 0.00622 | 0.07 | 0.04613 | −0.12 | 0.0004 |
| Myeloid. Not | 0.16016 | −0.05 | 0.14122 | −0.07 | 0.05153 | 0.01 | 0.70275 | 0.07 | 0.0369 |
| activated. | | | | | | | | | |
| Basophils | 0.00341 | 0.09 | 0.01196 | 0.01 | 0.6846 | 0.17 | 9.5E−07 | −0.14 | 3.6E−05 |
| T cells | 1.1E−07 | 0.17 | 1.5E−06 | 0.12 | 0.0007 | 0.03 | 0.35564 | −0.11 | 0.00114 |
| Lymphocytes | 0.0149 | −0.08 | 0.0223 | 0.03 | 0.42534 | −0.19 | 3.4E−08 | 0.14 | 9.8E−05 |
| T and B | | | | | | | | | |
| cells, mRNA | | | | | | | | | |
| translation | | | | | | | | | |
| NKTR, IL16 | 0.17912 | 0.05 | 0.1911 | 0.06 | 0.08086 | −0.05 | 0.13721 | 0.01 | 0.77421 |
| Unknown | 0.01398 | 0.08 | 0.02564 | 0.02 | 0.55253 | 0.08 | 0.02007 | −0.09 | 0.00694 |
| Monocyte | 0.00085 | −0.11 | 0.00137 | −0.01 | 0.76358 | −0.11 | 0.00199 | 0.12 | 0.00077 |
| TGFB1 | | | | | | | | | |
| CCR2+ | | | | | | | | | |
| Erythrocytes | 8.4E−10 | 0.15 | 1E−05 | 0.06 | 0.08531 | 0.09 | 0.00851 | −0.12 | 0.00036 |

In order to understand whether the three modules with positive correlation to the SLE cohort were related to other modules, the categories IFN PRR (black), plasma cell (magenta), and inflammatory myeloid (brown) were investigated further. The percentage of patients with positive eigengenes for each category was determined, and whether or not patients with positive eigengenes for one of these three gene modules were also positive for the other gene modules was determined. Table 16 demonstrates that patients positive for the IFN module were evenly split with regard to positivity of all other modules, except for the (myeloid not activated) (66%) and the (CD14 monocyte, TGFB1) modules (63%). Patients with positive eigengene values for the plasma cell module were also more likely to be IFN positive (72%), (CD14 TGFB1) positive (68%) and lymphocyte module positive (72%). Patients with inflammatory myeloid cell modules were likely to have positive eigengenes for the MicroRNA module (75%), (myeloid not activated) module (78%), basophils or granulocytes (67%), and negative eigengenes for lymphocytes (35%).

TABLE 16
Percentage of patients in each category with positive eigengene values

| | | | Percentage of Patients in Each Category with Positive Elgengene Values | | | | | | | | | | | | |
|---|---|---|---|---|---|---|---|---|---|---|---|---|---|---|---|
| | | Percent | | | | | | Myeloid | | | | | | | |
| | | Patients | IFN | Plasma | Myeloid | Micro | Plate- | not | Baso- | CD14, | Erythro- | No | NKTR- | Lympho- | T |
| | n | Positive | PRR | Cell | Inflam. | RNA | lets | activated | philis | TGFB1 | cyte | Identity | IL16 | cyte | cells |
| IFN PRR | 430 | 53% | | 57% | 55% | 53% | 47% | 66% | 48% | 63% | 40% | 37% | 53% | 54% | 39% |
| Module | | | | | | | | | | | | | | | |
| Positive | | | | | | | | | | | | | | | |
| Plasma Cell | 337 | 42% | 72% | | 37% | 35% | 37% | 53% | 36% | 68% | 34% | 38% | 54% | 72% | 39% |
| module | | | | | | | | | | | | | | | |
| Positive | | | | | | | | | | | | | | | |
| Inflam- | 384 | 47% | 61% | 33% | | 75% | 57% | 78% | 67% | 53% | 53% | 41% | 50% | 35% | 44% |
| matory | | | | | | | | | | | | | | | |
| Myeloid | | | | | | | | | | | | | | | |
| Module | | | | | | | | | | | | | | | |
| IFN Plus | 104 | 13% | | | | 70% | 42% | 87% | 57% | 72% | 32% | 22% | 51% | 50% | 29% |
| Myeloid Plus | | | | | | | | | | | | | | | |
| Plasma Cell | | | | | | | | | | | | | | | |
| IFN PRR | 132 | 16% | | | | 78% | 62% | 81% | 76% | 45% | 51% | 45% | 45% | 22% | 42% |
| Plus | | | | | | | | | | | | | | | |
| Myeloid | | | | | | | | | | | | | | | |
| IFN PRR | 140 | 17% | | | | 18% | 32% | 46% | 21% | 76% | 33% | 34% | 59% | 84% | 37% |
| Plus | | | | | | | | | | | | | | | |
| Plasma | | | | | | | | | | | | | | | |
| Cell | | | | | | | | | | | | | | | |
| Plasma | 22 | 3% | | | | 55% | 50% | 68% | 36% | 68% | 64% | 45% | 64% | 68% | 32% |
| Cell | | | | | | | | | | | | | | | |
| Plus | | | | | | | | | | | | | | | |
| Myeloid | | | | | | | | | | | | | | | |
| IFN Only | 53 | 7% | | | | 53% | 57% | 43% | 36% | 60% | 51% | 45% | 64% | 62% | 57% |
| PC Only | 71 | 9% | | | | 11% | 37% | 11% | 35% | 48% | 32% | 59% | 45% | 82% | 58% |
| Inflam | 126 | 16% | | | | 80% | 63% | 68% | 72% | 44% | 71% | 48% | 52% | 31% | 60% |
| Myeloid | | | | | | | | | | | | | | | |
| Only | | | | | | | | | | | | | | | |
| No IFN, PC | 162 | 20% | | | | 26% | 47% | 12% | 51% | 30% | 62% | 72% | 48% | 51% | 67% |
| or Myeloid | | | | | | | | | | | | | | | |

Further breakdown of the three categories with positive relationships to having SLE disease (versus control) demonstrated that patients who had positive eigengene values for all three categories were also likely to be positive for MicroRNA (70%), (Myeloid not activated) (87%), (CD14, TGFB1) (72%), and to have less positive eigengenes for erythrocytes (32%) and the T cell module (29%). Consideration of patients with positive eigengenes for two of the three modules showed that myeloid cells generally stayed together with the exception of the (CD14+TGFB1) module that seemed to sort with the IFN signature. Patients with positive eigengenes for inflammatory myeloid cells were generally positive for the MicroRNA signature, (myeloid not activated), basophils, and erythrocytes. Patients with positive eigengene values for plasma cells were likely to also be positive for lymphocytes (B and T cells) unless also positive for inflammatory myeloid cells. Perhaps most striking were the patients without positive eigengenes for any of the three modules positively correlated to SLE. These patients likely had positive eigengenes for the no identity module (72%) and T cells (67%). They were also likely negative for the MicroRNA module (26%+), myeloid not activated module (12%+), and CD14+TGFB1 monocyte (30%+). Whereas plasma cell and myeloid positive eigengenes were not mutually exclusive, they were unlikely to come together without also having an IFN signature (3%) and it was more common for these signatures to be alone (plasma cell+IFN 17% of patients, myeloid+IFN 16% of patients) than together with the IFN signature (13% of patients). These three patterns of signatures comprised 46% of the total patients (Table 16).

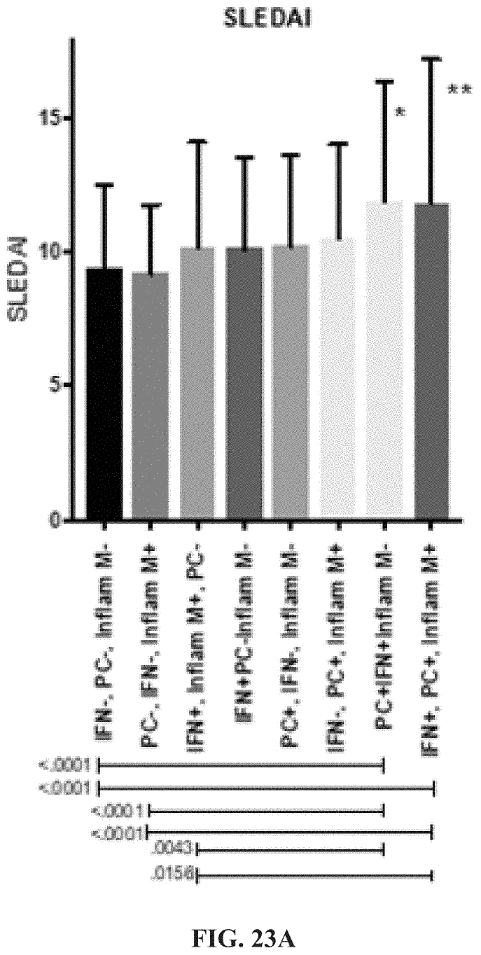

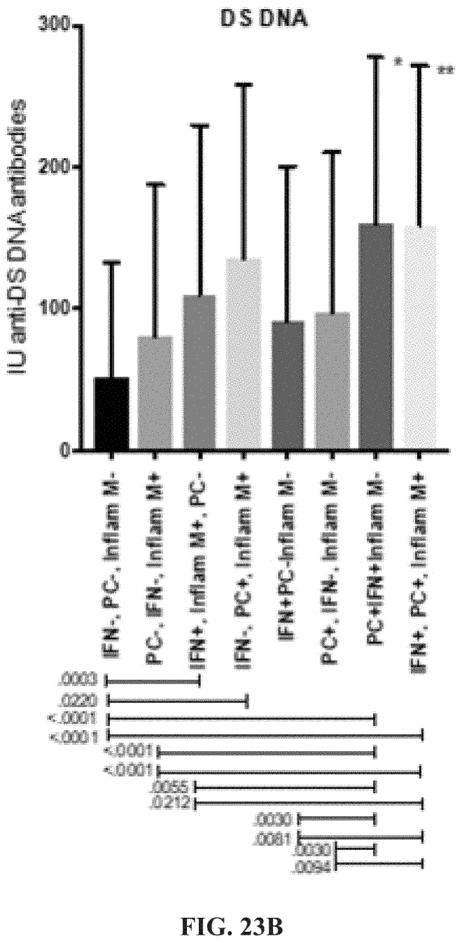

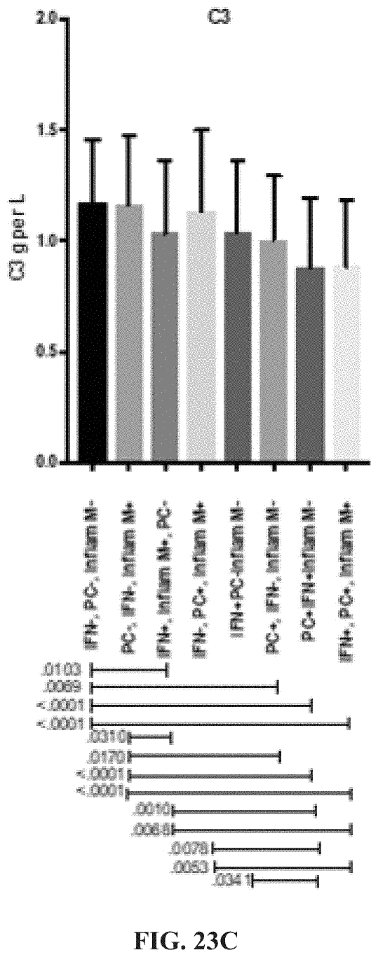

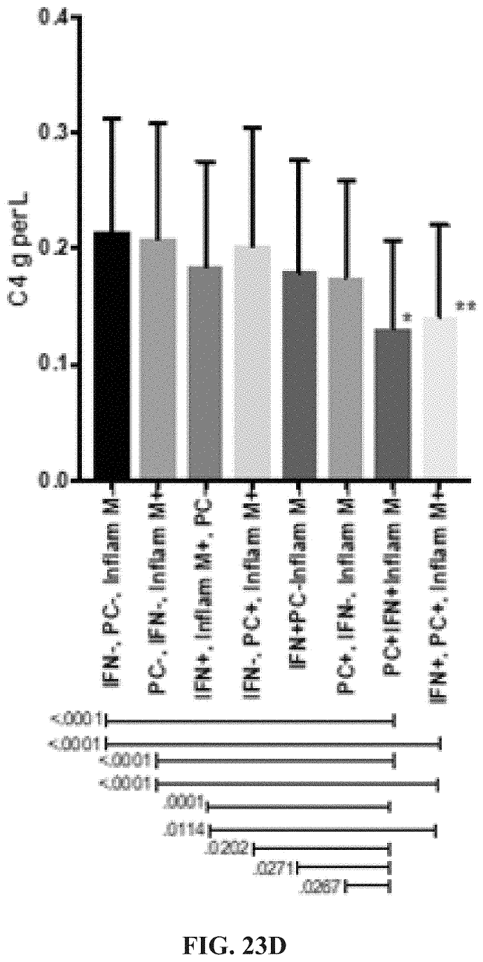

Next, the relationship between these modules and SLE disease activity was determined. The four disease measures considered were the SLEDAI, IU of anti-double stranded autoantibodies, g per L complement C3 and C4. As shown in FIGS. 23A-23D, for all disease measures, categories with plasma cells had higher measures of disease activity (increased SLEDAI, autoantibodies, Low C3, C4) than categories without, but the highest disease measures were when patients had positive eigengene values for both PC and the IFN signature.

FIGS. 23A-23D show the correlation between clinical measures of disease activity and WGCNA modules. Patients were divided into sub-groups based on their expression of positive eigengenes for each category. Significant differences between clinical traits were determined between group using PRISM v7 Tukey's multiple comparison test, and p values are shown between groups when less than or equal to 0.05.

The pink module had a negative correlation to the SLE cohort and included many T Cell Receptor J region chains and SNORAs and SNORDs. Its negative correlation with the presence of SLE may be used to help subdivide the patients further.

WGCNA was used to divide patients into distinct subsets based on the whether they had expression of plasma cell transcripts, IFN, PRR, and myeloid transcripts, or inflammatory myeloid transcripts. It also revealed that 20% of patients were negative for these transcripts, demonstrating that a significant proportion of patients entered into this clinical trial may have a type of non-immune cell mediated lupus. For example, these patients may be eliminated or excluded from lupus clinical trials for immune modulating drugs. Additionally, WGCNA clearly identified patients with only plasma cells but no inflammatory myeloid cells, and vice versa. Both of these signatures were likely to have an IFN signature as well. These signatures or endotypes may also allow for immune modulating drugs, which target plasma cells or myeloid cells, to be properly administered to patients with the matching blood signatures.

### Example 5: Molecular Endotyping Analysis for Identifying Subsets of Patients with Systemic Lupus Erythematosus Who are Candidates to be Enrolled in Clinical Trials and have a Propensity to Respond to Specific Drugs

Using methods and systems of the present disclosure, molecular endotyping analysis may be performed for identifying subsets of patients with Systemic Lupus Erythematosus who are candidates to be enrolled in clinical trials and have a propensity to respond to specific drugs.

Methods of molecular endotyping analysis may comprise performing Gene Set Variation Analysis (GSVA) on gene expression data with predefined gene sets, which may include genes descriptive of inflammatory or immune pathways or immune cell types. This yields a relatively small number of variables which are amenable to standard clustering methods such as k-means, k-medoids, or Gaussian mixture modeling (GMM). GMM may be advantageous over k-means because it considers the variance of each variable separately and is therefore less likely to be adversely affected by clusters of varying shapes and sizes. For each of these methods, clustering algorithms were applied with a range of possible numbers of clusters. Metrics such as the clustering silhouette and Bayesian Information Criterion (BIC) were used to select an optimal number of clusters. GMM analysis of GSVA scores from immunologically related modules in patients from the ILLUMINATE-1 and ILLUMINATE-2 trials indicated that the data was best fitted by four clusters.

The first cluster of patients was highly immunologically active, the second cluster was immunologically inactive, and the other two clusters displayed heterogeneous activation of immune cells and pathways. Patients in these clusters differed in their demographics, concomitant medications, and SLE manifestations. They also showed promising differences in their responses to tabalumab versus placebo. The cluster defined by myeloid cell activation showed little benefit from tabalumab, whereas the cluster defined by lymphoid cell activation trended toward a positive response to tabalumab. Interestingly, the immunologically inactive cluster also trended towards a positive response, partly because this group was the least responsive to placebo.

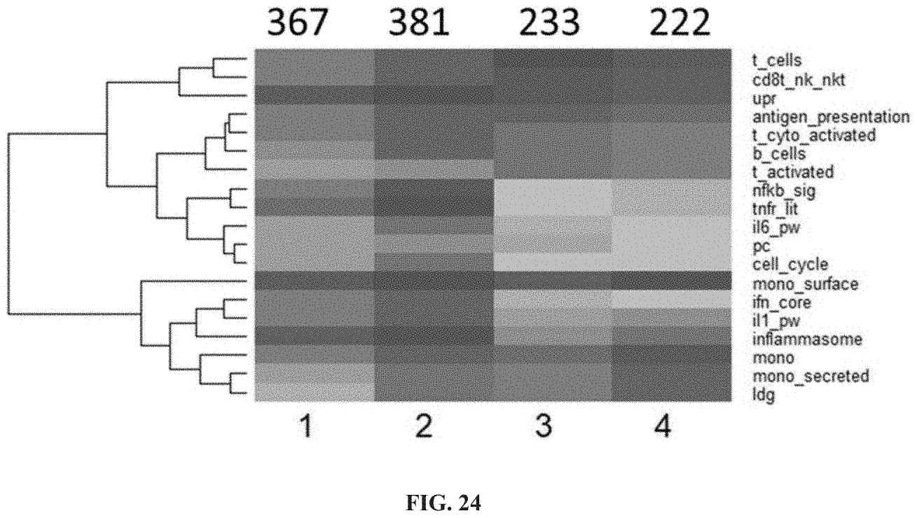

FIG. 24 shows mean GSVA scores of patients in each cluster defined by GMM. Numbers at the top denote the number of patients in each cluster.

The unbiased gene expression methods do not take prior knowledge of gene sets into account. In some embodiments, the method comprises unsupervised clustering of gene sets generated by WGCNA, as described above. The modules generated by WGCNA can then be used to perform k-means, k-medoids, or GMM clustering of patients. In some embodiments, a search is performed for genes whose expression values are bimodally distributed (preliminary analysis of ILLUMINATE data indicates there are roughly 40 of these genes, mostly IFN-related). These genes are then investigated with clustering methods. In some embodiments, non-linear dimensionality reduction is performed on gene expression data with an autoencoder neural network, and then subjects are clustered based on the resulting latent variables. A particular kind of autoencoder, termed a Gaussian mixture variational autoencoder (GMVAE), constrains the latent variables to be generated by Gaussian mixtures. The gene expression data activates the components of the Gaussian mixtures, which in turn activate the latent variables, which are decoded to reconstruct the gene expression input. A GMM may then be fitted to the latent space to perform clustering; alternatively, subjects may be assigned to clusters based directly on the mixture probabilities.

Clustering methods based on the subjects' clinical parameters also may be used to generate meaningful subsets. Combinations of factors such as age, ancestry, SLE manifestations, and concomitant medications allow for clustering of trial subjects. Methods such as k-medoids may be applicable to categorical data sets. GMVAEs, which are often employed to cluster image data, may be used to process binary clinical variables because these variables are analogous to activated or deactivated pixels in an image.

GMVAE clustering of clinical variables from patients in the ILLUMINATE trials was performed, and five clusters of patients were identified (Table 17). A GMVAE with two latent dimensions was trained on 13 clinical variables. The model correctly reconstructed an average of 10 traits, indicating strong performance even with a relatively low number of samples by neural network standards. This approach was used to identify five patient clusters. There is a very similar cluster of young patients with aggressive disease that respond poorly to placebo (Chi-square p value=0.16).

TABLE 17
Average patients in each cluster

| | | | | Anti- | Low | | | | | | | | |
|---|---|---|---|---|---|---|---|---|---|---|---|---|---|
| Size | SLEDAI | Age | Alopecia | dsDNA | Comp. | Ulcers | Antimal. | Cortico. | Immuno. | NSAID | Q2W | Q4W | Placebo |
| 218 | 11 | 42 | 62% | 67% | 35% | 35% | 65% | 82% | 39% | 30% | 41% | 44% | 38% |
| 405 | 12 | 37 | 59% | 98% | 94% | 35% | 63% | 98% | 50% | 13% | 41% | 51%* | 30% |
| 242 | 8 | 45 | 76% | 11% | 2% | 25% | 81% | 74% | 31% | 23% | 46% | 47% | 41% |
| 110 | 11 | 39 | 49% | 92% | 51% | 22% | 57% | 80% | 57% | 26% | 47% | 33% | 31% |
| 228 | 9 | 46 | 50% | 18% | 14% | 52% | 59% | 21% | 25% | 71% | 41% | 40% | 38% |

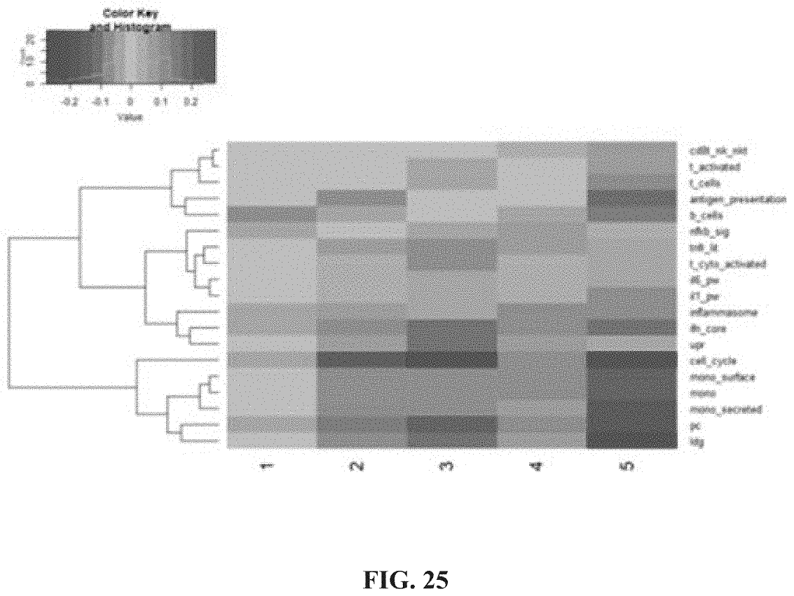

The patients in clusters 3 and 5 did not have anti-dsDNA or low complement, and were treated with antimalarials and either corticosteroids or NSAIDs. These patients did not show significant benefit from tabalumab compared to placebo. The other three clusters were more likely to have anti-dsDNA and low complement. Cluster 4, which included 171 patients treated with corticosteroids and immunosuppressives, showed a trend toward positive response to tabalumab (SRI-5 response rates: Q2 W 47%, Q4 W 33%, Placebo 31%). Cluster 2, which was treated with antimalarials and corticosteroids, achieved significant results (SRI-5 response rates: Q2 W 41%, Q4 W 51%, Placebo 30%). FIG. 25 shows gene expression of subjects in groups defined by GMVAE. GSVA analysis of the patients in these clusters showed that the patients without serological SLE activity (clusters 3 and 5) also did not show immunological activity by gene expression, whereas the other clusters did show immunological activity.

These approaches demonstrate that patients can be automatically distinguished or stratified into distinct groups, clusters, or subsets, via analysis of their gene expression data, based on factors such as whether a given clinical trial (e.g., for a lupus drug) is more or less likely to succeed for a particular patient. Certain subsets of subjects were shown to respond to treatment at substantially different rates from the other subjects in the study. However, small deviations toward better response to active treatment and worse response to placebo can be combined to produce significant results. Subsets have been successfully identified which are a fraction of the size of the original trials yet still see significant improvement from active treatment compared to placebo. Also, subsets of patients may be identified who achieve little to no benefit from active treatment and ought to be excluded from enrollment in clinical trials. In the ILLUMINATE trials, subsets were identified based on characteristics beyond those that were originally tested for an effect on the outcome. For example, it may seem intuitive to divide subjects in an anti-B-cell activating factor trial on the basis of anti-dsDNA seropositivity, but this failed to explain the failure of the trial. In the analysis results presented herein, the trial succeeded in a cluster of patients with anti-dsDNA, low complement, and concomitant corticosteroids but failed in clusters of patients that were more defined by concomitant use of immunosuppressives. These results demonstrate that complex combinations of factors may be used to more effectively and successfully subdivide patients (e.g., into responder and non-responder groups).

### Example 6: Ancestry Influences the Gene Expression Profile in Systemic Lupus Erythematosus (SLE) and Contributes to Gene Expression Heterogeneity in Lupus Patients

Systemic Lupus Erythematosus (SLE) generally refers to a complex autoimmune disease, which has both sex and ancestral bias in affected patients. Gene expression analysis may reveal complex heterogeneity between SLE patients, and the contribution of ancestry, drugs, and SLE manifestations to this heterogeneity were determined. Gene expression analysis between female disease-matched SLE patients of African, European, and Native American ancestry revealed thousands of differentially expressed (DE) transcripts between ancestries, but none within a single ancestry. African, European, and Native ancestry SLE patients had significantly different cellular contributions to gene expression, and these differences were found to be related to significantly different percentages of patients in each ancestry with specific signatures. Gene Set Variation Analysis (GSVA) showed an increase in plasma cells, B cells, and T cells in the majority of African ancestry patients and an increase in myeloid cell transcripts in most European and Native American ancestry patients. The treatment of SLE patients with drugs, such as corticosteroids and immunosuppressives, significantly changed their gene expression and contributed to the disparate signatures between and within ancestries. Autoantibodies and low complement, but not other clinical features of SLE, were also significantly associated with the gene expression in European and Native American ancestry SLE patients and to a lesser degree in African ancestry SLE patients. Further, differences between African and European ancestry SLE patients were found to be similar to those between healthy people of these ancestries. These ancestry-specific gene expression profiles provide a specific transcriptomic background upon which the SLE patient gene expression pattern can be built.

Systemic Lupus Erythematosus (SLE) generally refers to a complex autoimmune disease affecting mostly women (9:1) and characterized by autoantibodies to DNA and nuclear proteins leading to immune complex formation, complement deposition, and immune damage in multiple organ systems. Heterogeneity in ancestral prevalence, disease severity, organ involvement, and response to treatment can be observed; however, an explanation had not been fully delineated. Whereas the disease may be most prevalent in Asians and people of African-Ancestry (AA), a disproportionate number of clinical trials may be focused on the European Ancestry (EA) population. Further, Native people of North American ancestry may have earlier onset of disease and more organ involvement. In some cases, increased active disease, organ involvement, and autoantibody levels may be observed for AA compared to EA patients, and increased mortality may be observed for AA patients. At the cellular level, the AA population may have more activated B cells and B cell receptor signaling than the EA population. There may be differences in responses of both innate immune cells as well as lymphocytes, suggesting that ancestral differences in immune cells may contribute to the different disease course and incidence between populations. Also, there may be ancestry-related differences in response to therapy across individual patients. For example, AA SLE patients may respond better to B cell depletion therapies than Caucasian patients, but they may display lower responses to anti-BAFF treatment in Phase III clinical trials. Higher serum levels of BAFF in AA SLE patients may suggest that higher doses of the biologic may be necessary in AA patients, and that underlying genetic differences between AA and EA SLE patients may be accounted for in determining treatment decisions. There may be different genetic components contributing to disease development and progression in different ancestral populations. For example, transancestral genetic mapping may demonstrate a multigenic effect in SLE that differs according to ancestral background, suggesting a heterogeneous genetic component to disease activity. Unfortunately, many multigenic Genome Wide Association Study (GWAS) differences between AA and EA may be present in non-coding regions, thereby making extrapolation to differences in disease severity challenging.

Heterogeneity in SLE gene expression signatures may be observed for the IFN-stimulated genes. SLE patient gene expression differences may be investigated by creating modules of genes over-represented in pediatric SLE patients. Although expression of some modules may be correlated with changes in disease activity, it may be difficult to reconcile disease activity as measured by SLE Disease Activity Index (SLEDAI) and gene expression signatures in patients. For example, an attempt to group lupus patients in 158 pediatric SLE patients may suggest as many as seven different types of lupus. Increased plasmablasts may be detected in AA and increased myeloid signatures may be observed in some EA and Hispanic SLE patients, suggesting that there may be an ancestral basis to explain some of the heterogeneity in SLE gene expression signatures. The many different SLE organ manifestations may also contribute to the heterogeneity in gene expression signatures. The low-density granulocyte (LDG) signature observed in SLE PBMC may correlate with skin and vasculitis manifestations. Further, neutrophil signatures may correlate with progression to active lupus nephritis in pediatric SLE patients. An association between the IFN signature and skin involvement, anti-double-stranded DNA autoantibodies (anti-dsDNA), low complement (Low C) and musculoskeletal SLEDAI manifestations may also be observed.

Whole blood transcriptomes and gene expression analysis may be performed to assess the pattern of abnormal representation of thousands of genes simultaneously, thereby deducing the underlying abnormalities. Moreover, this approach can be used to develop an understanding of the association of ancestry, standard of care (SOC) therapy, and SLE manifestations. Here, the contribution of ancestry, SOC drug therapy, and SLE manifestations to the blood gene expression profile of subjects with SLE was determined. Although some study may assume the transcriptomic differences between SLE patients and healthy controls (HC) are related to the disease, these results provide strong evidence that much of the gene expression signature measured between SLE patients and HC is related to patient ancestry and SOC drug regimens, thereby resulting in alterations in the proportions of hematopoietic cells, cellular processes, and signaling pathways detected.

Significant Ancestral Gene Expression Differences in SLE Patients

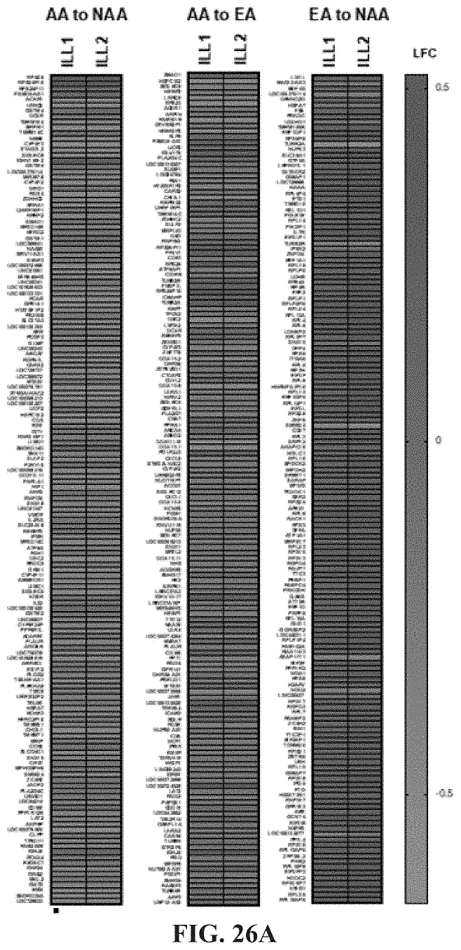

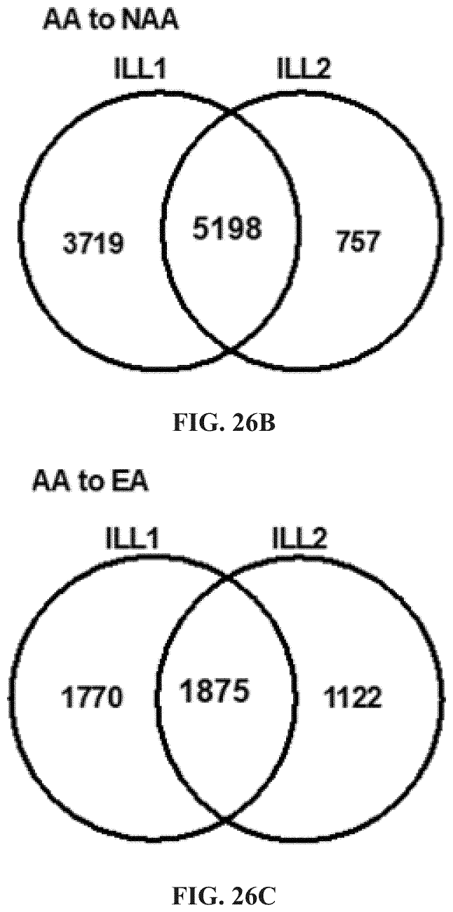

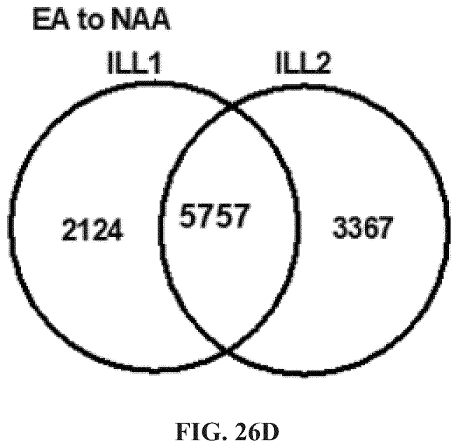

In order to determine ancestral contributions to gene expression signatures in whole blood (WB), two large phase 3 clinical trial databases with microarray analysis at baseline were analyzed (GSE88884, as described by Hoffman, 2017, which is incorporated by reference herein in its entirety). The Illuminate 1 (ILL1) and Illuminate 2 (ILL2) clinical trials had microarray expression data for 1,566 female patients of self-described ancestry as follows: AA (n=216), EA (n=1,118), and Native American Ancestry (NAA; mostly from South America, n=232; top three countries of origin Peru (n=81), Ecuador (n=30), and Guatemala (n=27)); male patients and patients of multiple, Asian, and other ancestries were removed to avoid contributions of gender differences and low numbers of patients, respectively. Ancestral backgrounds were split evenly between the ILL1 and ILL2 datasets, allowing for a training and test set to determine bulk gene expression differences. Entry criteria for the trials required a positive anti-nuclear autoantibody (ANA) titer and a minimum disease activity of 6, as determined by the SLE Disease Activity Index (SLEDAI). Disease activity was similar among ancestries, as was percentage of patients with anti-dsDNA (Table S1). The trials excluded patients with progressive lupus nephritis and entered only one patient with central nervous system manifestations. Most female patients recruited had a mixture of six SLE manifestations: arthritis (86.4%), anti-dsDNA (57.5%), low complement (Low C, 40.0%), alopecia (58.9%), rash (68.3%), and mucosal ulcers (31.7%) (Table S2). Gene expression differences were first determined by carrying out limma differential expression (DE) analysis of AA, EA, and NAA SLE patients to each other. At a false discovery rate (FDR) of 0.05, thousands of DE transcripts were determined for each ancestry compared to the others for the ILL1 dataset (FIGS. 26A-26D). As a control, each ancestral background was randomized into two separate groups five separate times, and DE to patients of the same ancestral background was assessed. No DE transcripts were found, even at a less stringent FDR of 0.2. DE analysis of ILL2 SLE patients of AA, EA, and NAA SLE patients to each other yielded similar results to ILL1, indicating thousands of DE transcripts between ancestries at an FDR of 0.05 (FIGS. 26A-26D). Importantly, the patterns of ancestry-related DE genes were comparable in ILL1 and ILL2 (FIGS. 26A-26D).

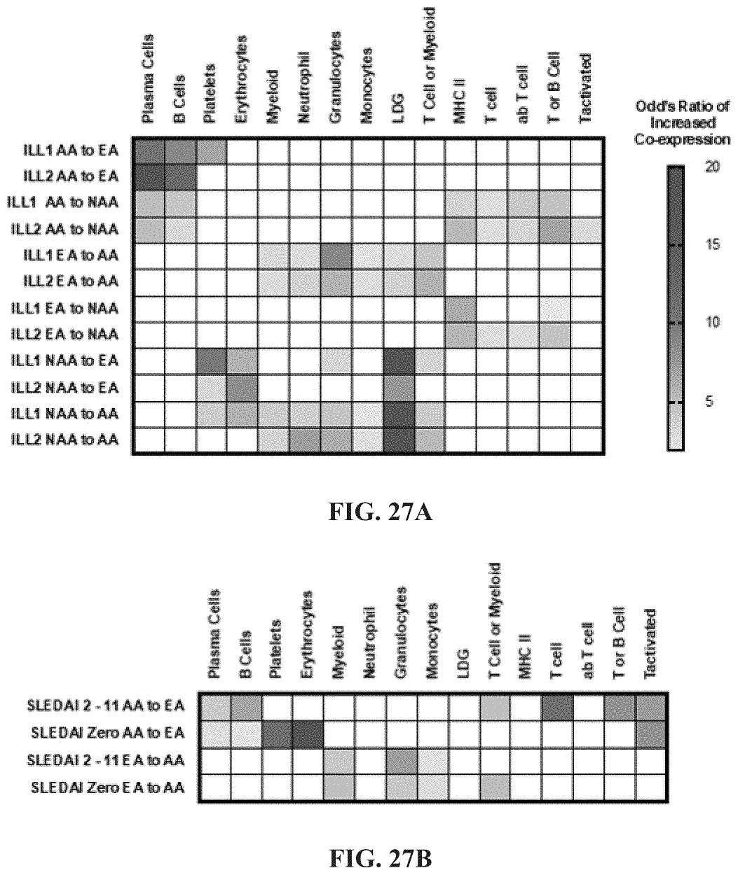

In order to interpret the biological meaning of the ancestral gene expression differences, I-scope, a tool for determining the likely hematopoietic cell type in bulk datasets, was used to determine whether there were cellular differences between SLE patients of different ancestral backgrounds. I-Scope demonstrated a relative predominance of plasma cells and B cells in AA patients, and of myeloid cells in EA and NAA patients. In EA SLE patients, transcripts for monocytes and low-density granulocytes (LDGs) were enriched compared to AA SLE patients, whereas T cell and MHC class II transcripts were enriched in EA patients compared to NAA patients. NAA patients had increased myeloid signatures, including transcripts associated with monocytes, LDGs, and neutrophils compared to both AA and EA patients (FIG. 27A). Thus, the same ancestral-based cellular enrichments were found for the ILL1 and ILL2 dataset, and the transcripts signifying these cellular categories were remarkably similar between the ILL1 and ILL2 datasets. These results indicated a meaningful difference in gene expression profiles of SLE subjects with similar disease severity but of different ancestries.

Next, Gene ontology (GO) biological pathway and Biologically Informed Gene Clustering (BIG-C) (Labonte et al., 2018) enrichment of molecular pathways (Fisher's Exact p<0.05) in AA, EA, or NAA patients was performed, and results supported the conclusions of the I-scope analysis. GO biological pathways demonstrated increased innate immune response and neutrophil chemotaxis in EA and NAA SLE patients compared to AA patients, and increased immunoglobulin transcripts (in GO categories complement activation and regulation of immune response) in AA compared to EA and NAA. There were no GO biological pathways enriched in EA patients compared to both AA and NAA patients. BIG-C analysis revealed that AA patients had increased immune cell surface, immune signaling, and MHC II compared to both NAA and EA patients. AA patients also manifested increased IFN stimulated genes, chromatin remodeling, fatty acid biosynthesis, and the unfolded protein response compared to EA patients. NAA patients had increased immune cell surface, immune signaling, MHC I, autophagy, inflammasome and pattern recognition receptors, anti-apoptosis, and ROS protection compared to both AA and EA patients. NAA patients had increased IFN stimulated genes, transporters, unfolded protein response and integrin pathway compared to EA patients. Similar to GO biological pathways, there were no increased BIG-C categories for EA patients compared to both AA and NAA patients. Gene categories up-regulated in EA patients compared to AA patients included immune cell surface, autophagy, ROS protection, lysosome, and glycolysis. AA and EA patients shared increases in a number of categories compared to NAA patients indicating these processes were likely decreased in NAA patients compared to both AA and EA patients; these included mitochondrial DNA to RNA, mRNA translation, mRNA splicing, MicroRNA processing, TCA cycle, oxidative phosphorylation, and proteasome.

The 798 ILL1 and 768 ILL2 SLE patients were analyzed separately and yielded similar results, even at the individual gene level. To rule out the possibility that these findings could not be extrapolated to other SLE datasets, and to confirm the finding that ancestral differences were significantly contributing to the heterogeneity in gene expression signatures, SLE dataset GSE45291 was also analyzed. 73 AA and 71 EA SLE patients with the same range of SLEDAI scores (2-11), similar mean SLEDAI (AA 3.78+/−2.46; EA 3.53+/−2.08), and mode of SLEDAI (2), were analyzed by Linear Models for Microarray Data (limma) DE analysis, and results indicated that 859 transcripts were increased in AA patients compared to EA patients, and 955 transcripts were increased in EA patients compared to AA patients (FDR 0.05).

Similar to the results using the ILL1 and ILL2 datasets, EA SLE patients were enriched for transcripts associated with myeloid cells (FIG. 27B), and AA SLE patients were enriched for transcripts associated with plasma cells, B cells, and T cells (FIG. 27B).

GO biological pathway analysis demonstrated increased transcripts associated with chemotaxis, TLR signaling, and proteins which may be phosphorylated in EA, and increased transcripts for regulation of immune response, translation, T cell co-stimulation, complement activation, and BCR signaling in AA SLE patients.

BIG-C analysis showed increased immune cell surface, immune signaling, oxidative phosphorylation, mRNA translation, ubiquitylation and ER in AA and increased autophagy, inflammasome, glycolysis, lysosome, endosome, immune cell surface, and intracellular signaling in EA patients. DE analysis of SLE patients with inactive disease (SLEDAI of zero), including 25 AA and 75 EA patients, also revealed significant DE transcripts: 470 increased transcripts in EA patients and 258 increased transcripts in AA SLE patients (FDR of 0.05).

I-scope analysis showed a similar pattern of increased transcripts related to myeloid cells in EA patients, including CLEC4D, CXCL1, CXCL8, FCGR3B, FGL2, LTB4R, BPI, CAMP, IL17RA, MMP9, SIGLEC9, BMX, ITGAM, FPR1, and to plasma cells and B cells in AA patients, including transcripts for IGKC, IKGV4-1, IGLC1, IGLJ3, and JAKMIP1, even though the number of these cell-specific transcripts were decreased compared to patients with higher SLEDAI values (FIGS. 27A-27B). GO biological pathway analysis demonstrated increased glucose metabolism, small GTPase signal transduction, and vesicle fusion in EA patients, and increased membrane components, heme biosynthesis, microtubule, and secreted protein transcripts in AA patients with very low disease activity. Further, BIG-C analysis demonstrated immune cell surface, cytoskeleton, MHC II, and mitochondria increased in AA patients, and TCR cycle, lysosome, endosome, and ubiquitylation upregulated in EA patients. Thus, DE analysis of 4 SLE datasets comprising 1,810 female SLE patients demonstrated significant ancestral components to the whole blood gene expression profile, and some of these gene expression differences were observed to be independent of disease activity.

Differences in Gene Expression Between Ancestries were Associated with Significantly Different Percentages of Patients with Particular Signatures

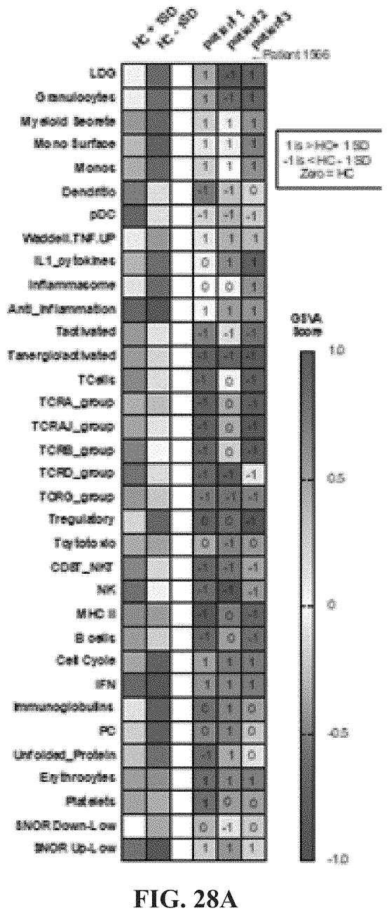

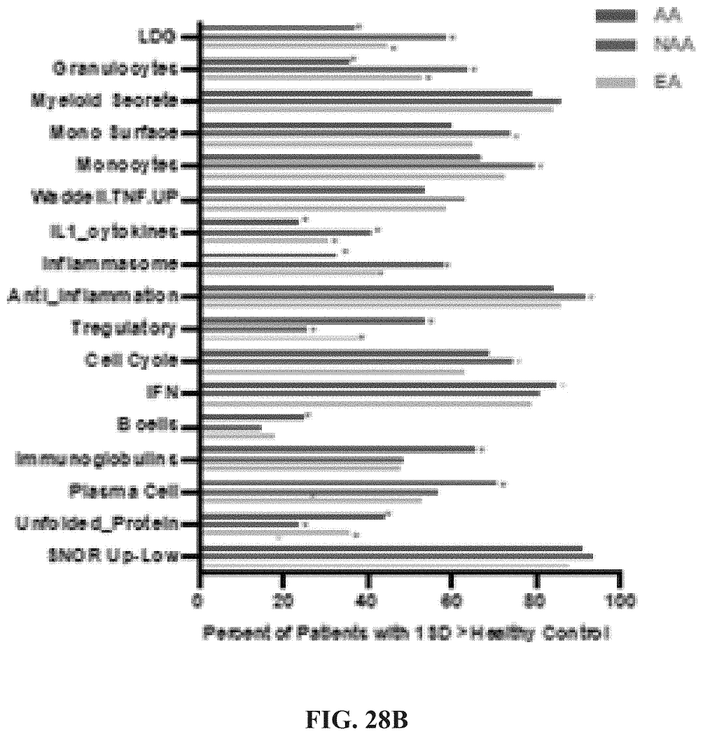

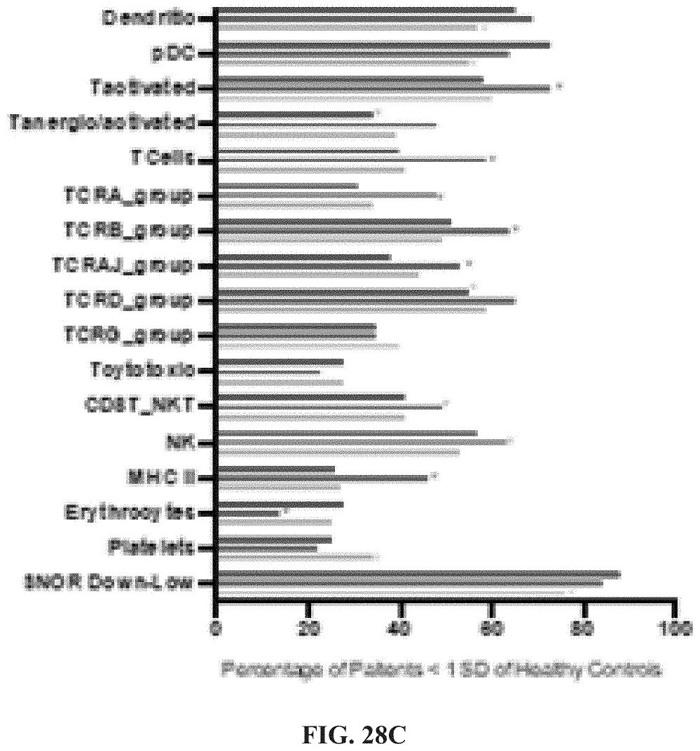

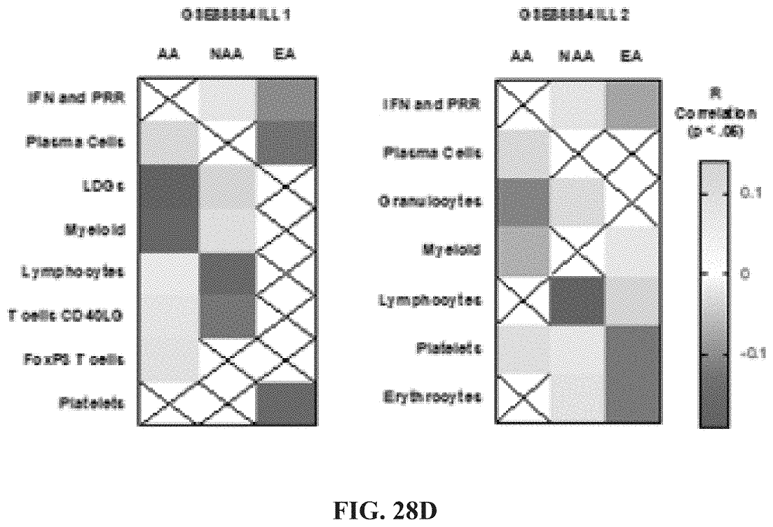

Using the population gene expression analysis was useful for finding signatures that were significantly different for groups of patients of a specific ancestry. Further, a possibility that features of individual subjects, such as therapy and/or specific disease manifestations, may have contributed to such DE was ruled out, which may be important since ancestral groups may differ in these features. To address this, gene set variation analysis (GSVA) was employed to compare enrichment of 34 modules of genes corresponding to lymphocytes, myeloid cells, cellular processes, as well as groups of all the T Cell Receptor (TCR) and immunoglobulin (Ig) genes found on the Affymetrix HTA2.0 array. GSVA calculates enrichment scores using the log 2 expression values for a group of genes in each SLE patient and healthy control and normalizes these scores between −1 (no enrichment) and +1 (enriched). When many genes of a particular cell type or process are co-expressed, GSVA roughly reflects cell counts (FIG. S2). GSVA enrichment scores were calculated for the set of 1,566 female SLE patients and 17 female HC from the ILL1 and ILL2 datasets (GSE88884). The average plus or minus 1 standard deviation (SD) for the healthy controls was used to determine whether a patient had an increased, decreased, or similar signature compared to HC (FIG. 28A).

GSVA results demonstrated that the differences between the ancestry groups were related to the significantly different percentages of patients with particular signatures. All three ancestry groups had significantly different frequencies of patients (p<0.01, Fisher's Exact Test) with enrichment of the LDG, granulocyte, IL1 cytokine, and inflammasome signatures. NAA patients had the highest percentage of patients with these signatures, followed by EA patients, and AA patients had the lowest. NAA patients also had significantly more patients with monocyte cell surface and monocytes than AA patients; however, interestingly, signatures for myeloid secreted proteins, which included complement components, TNF, and CXCL10, were not different between the three ancestry groups. The AA patient group had significantly more patients with B cell, Ig, plasma cell, and T regulatory (IKZF2, FOXP3) signatures compared to EA and NAA patients. The NAA patient group had significantly fewer patients with T cell associated signatures compared to both EA and AA patients. The EA patient group had significantly fewer patients with dendritic and pDC signatures decreased compared to controls. The percentage of AA patients with IFN signatures was higher than that of EA patients (Fisher's exact p=0.04), but differences in overall percentages only ranged from 79% positive (EA) to 85% positive (AA). The AA and NAA patient groups had significantly more SLE patients with platelet and erythrocyte enrichment than EA patients, and significantly fewer patients with decreased erythrocyte and platelet GSVA scores compared to EA patients (FIGS. 28B-28C).

An orthogonal approach using weighted gene co-expression network analysis (WGCNA) was used to confirm the association of ancestry with cellular signatures. WGCNA of GSE88884 ILL1 and ILL2 was performed separately, and results demonstrated a significant (p<0.05) positive association by Pearson correlation of AA ancestry to plasma cell, T cell, and FOXP3 T cell modules, as well as a significant negative correlation to granulocyte and myeloid cell WGCNA modules. NAA ancestry had positive correlations to IFN, granulocyte, platelet, and erythrocyte modules, and negative correlations to T cell and lymphocyte modules. EA ancestry was positively correlated to one myeloid cell module and negatively correlated to IFN, plasma cell, platelet, and erythrocyte modules (FIG. 28D). These analyses confirmed the findings from the DE and GSVA analysis.

SOC Therapy is Associated with Changes in Gene Expression Profiles

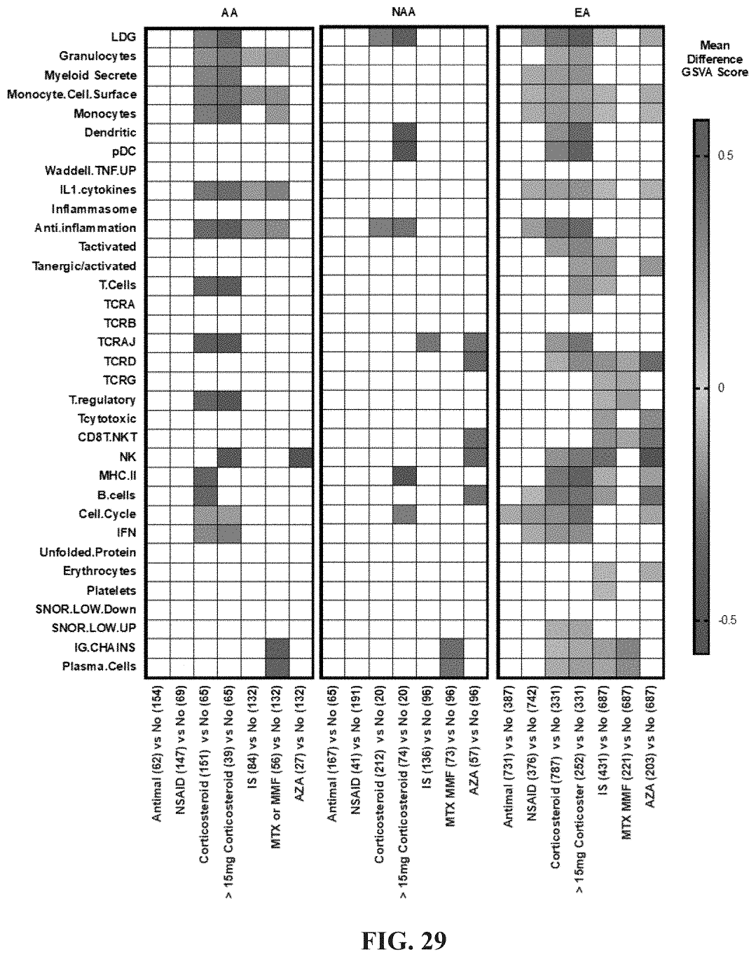

All SLE patients in these analyses were on SOC drug therapy, and the heterogeneity observed in gene expression signatures between ancestral backgrounds may have been influenced by different drug regimens. In order to determine the effect of SOC drugs on patient gene expression signatures, patients on specific therapies were compared to patients not receiving the therapies for the 34 cell type and process modules. Within ancestral groupings, patients taking corticosteroids had significantly (Sidek's multiple comparisons test) increased LDG (AA, EA, and NAA, with p<0.0001) and anti-inflammation (AA, EA, and NAA, with p<0.0001) GSVA scores compared to patients of the same ancestry not taking the drugs, demonstrating that these signatures were strongly influenced by corticosteroid usage. Additionally, both AA and EA patients receiving corticosteroids had significant enrichment for granulocytes (AA, p=0.0009; EA, p=0.005), myeloid secreted (AA, p=0.0001; EA, p<0.0001), monocyte cell surface (AA and EA, p<0.0001), monocytes (AA and EA, p<0.0001), cell cycle (AA, p=0.04; EA, p<0.0001) and the IFN signature (AA, p=0.001; EA, p<0.0001). The effect of corticosteroids on myeloid signatures was further amplified at corticosteroid doses greater than 15 mg/day. Immunosuppressive therapy (e.g., IS, azathioprine (AZA), mycophenolate mofetil (MMF), or methotrexate (MTX)) did not have a consistent effect on all three ancestry groups. However, IS increased monocyte cell surface (EA, p=0.0013; AA, p=0.0103) and IL1 (EA, p=0.03; AA, p=0.0168) in AA and EA patients. When IS therapy was restricted to just MMF and MTX, there was a consistent decrease across all three ancestry groups in plasma cell (AA, p=0.0087; EA, p<0.0001; NAA, p=0.0130) and immunoglobulin (AA, p=0.0026; EA, p<0.0001; NAA, p=0.0168) GSVA scores. AZA treatment yielded significantly decreased NK cell GSVA scores (AA, p=0.0004; EA, p<0.0001; NAA, p=0.002) in all three ancestry groups and also significantly decreased T cytotoxic (EA and NAA, p<0.0001) and B cells (EA and NAA, p<0.0001) in NAA and EA ancestries. EA patients receiving NSAIDs compared to all other treatments had decreased LDG (p<0.0001) and anti-inflammation signatures (p=0.0053), whereas anti-malarial drugs had no significant effect on enrichment scores of the 34 cell type and process modules (FIG. 29).

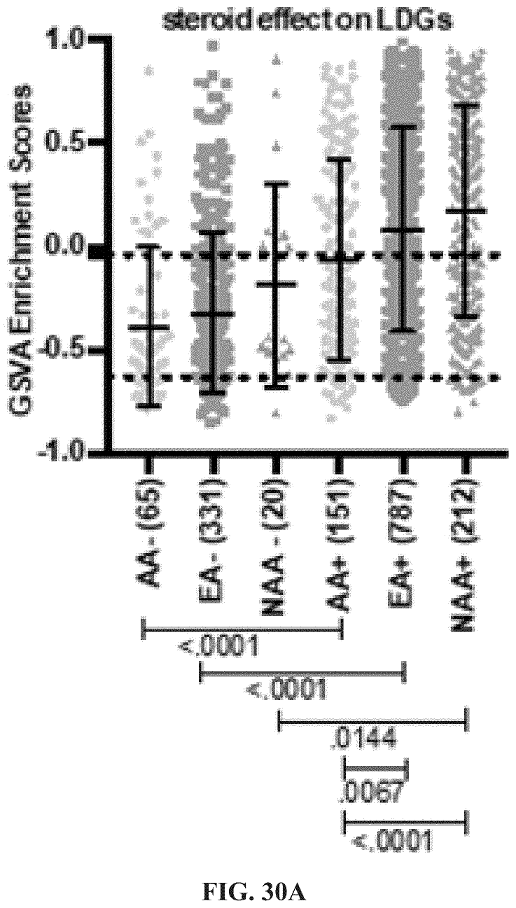

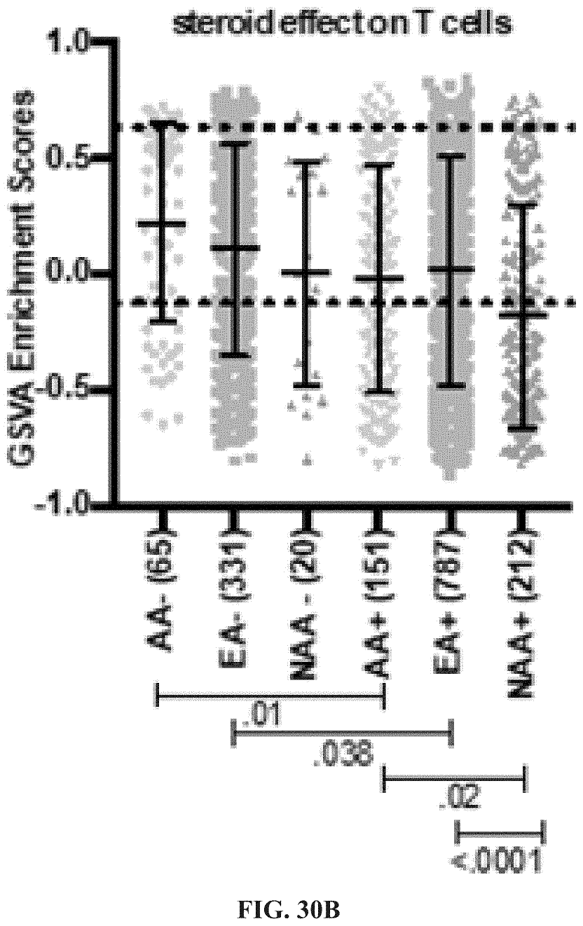

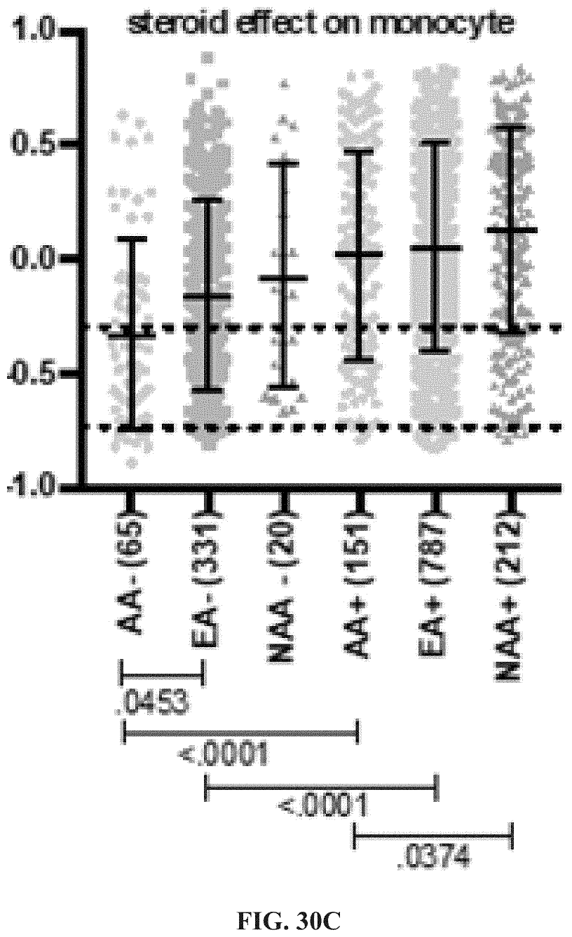

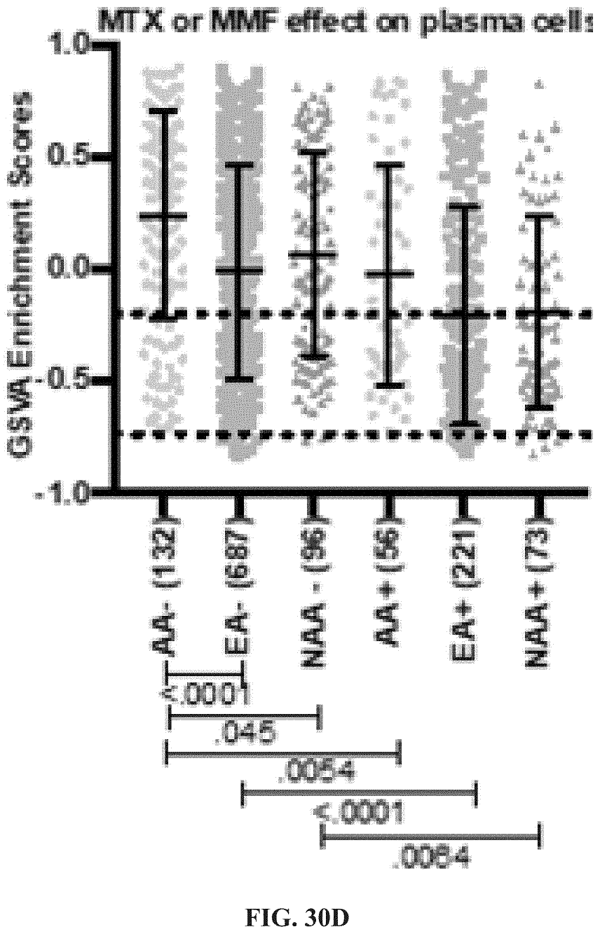

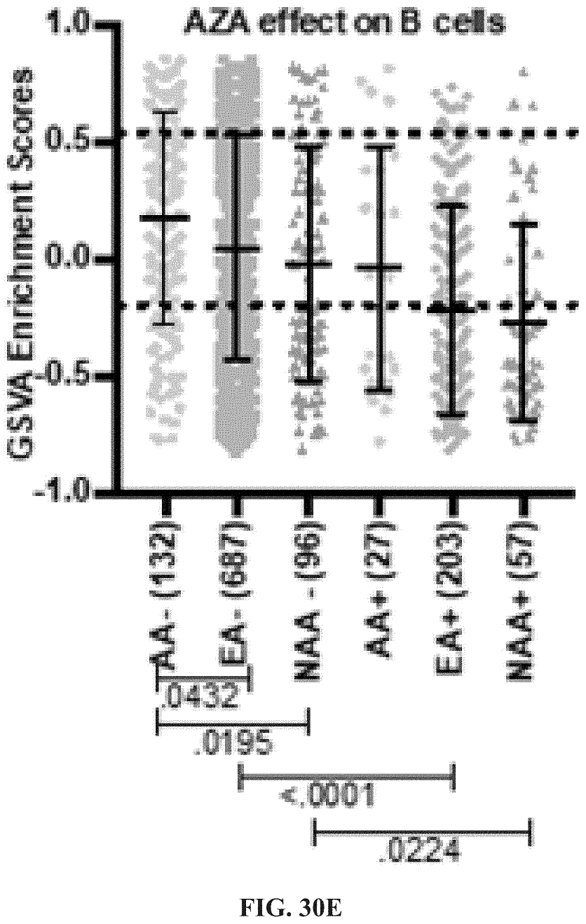

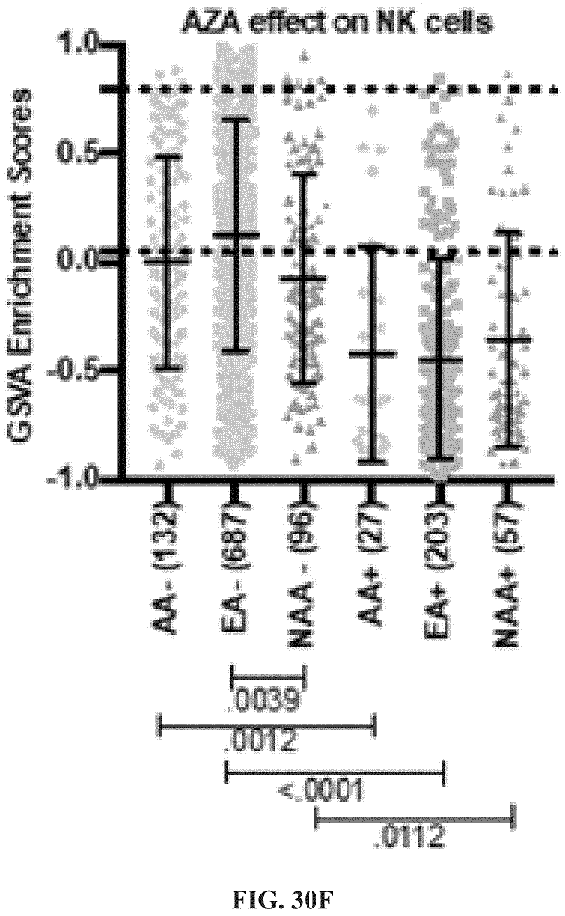

To demonstrate that these treatment differences were sufficient to account for the ancestral gene expression differences, signatures were compared between patients on the same drug regimens. Almost all NAA SLE patients were receiving corticosteroids (92%; n=214/232) compared to 70% of AA (n=152 out of 216) and EA (n=787 out of 1,118) patients, and NAA patients were also more frequently taking immunosuppressive drugs (58%) compared to AA (39%) and EA (39%) patients. Comparison of LDG, monocyte, and T cell GSVA scores for patients with or without corticosteroids demonstrated that the corticosteroids were the largest contributor to the differences between patient LDG, monocyte, and T cell scores, but that AA patients still had lower LDG and monocyte scores and NAA patients still had lower T cell scores in the absence of corticosteroids (FIGS. 30A-30C). MTX and MMF significantly lowered plasma cell GSVA scores, but did not negate the increased plasma cells determined for AA patients versus EA and NAA patients (FIG. 30D). Compensating for AZA treatment also did not offset the increased B cells in AA SLE patients (FIG. 30E) or the difference in NK cells between EA and NAA SLE patients (FIG. 30F).

Dataset GSE45291 also had current drug information available for the gene expression data; therefore, GSVA enrichment scores were determined for the 34 cell and process modules, and differences between different drug treatments were determined. Corticosteroids increased LDG, monocyte, and anti-inflammation GSVA enrichment scores, MTX and MMF decreased plasma cell GSVA enrichment scores, and AZA decreased NK and B cell enrichment scores (FIG. S3), in support of the data generated from dataset GSE88884.

Autoantibodies and Complement Levels, but not Clinical Features were Associated with Gene Expression Profiles

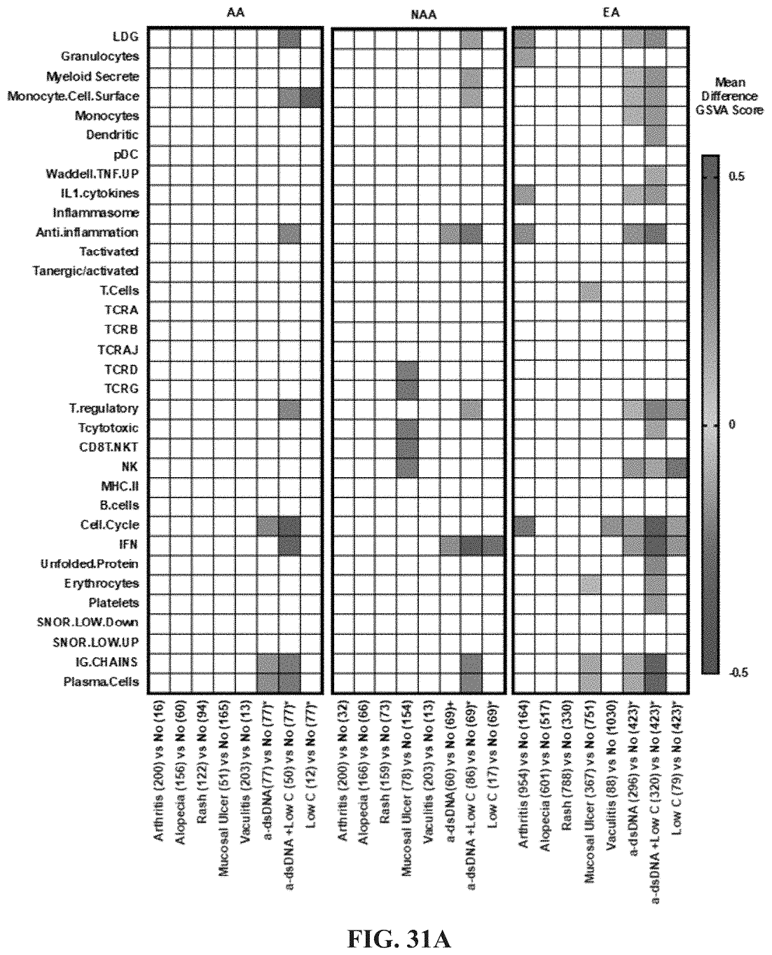

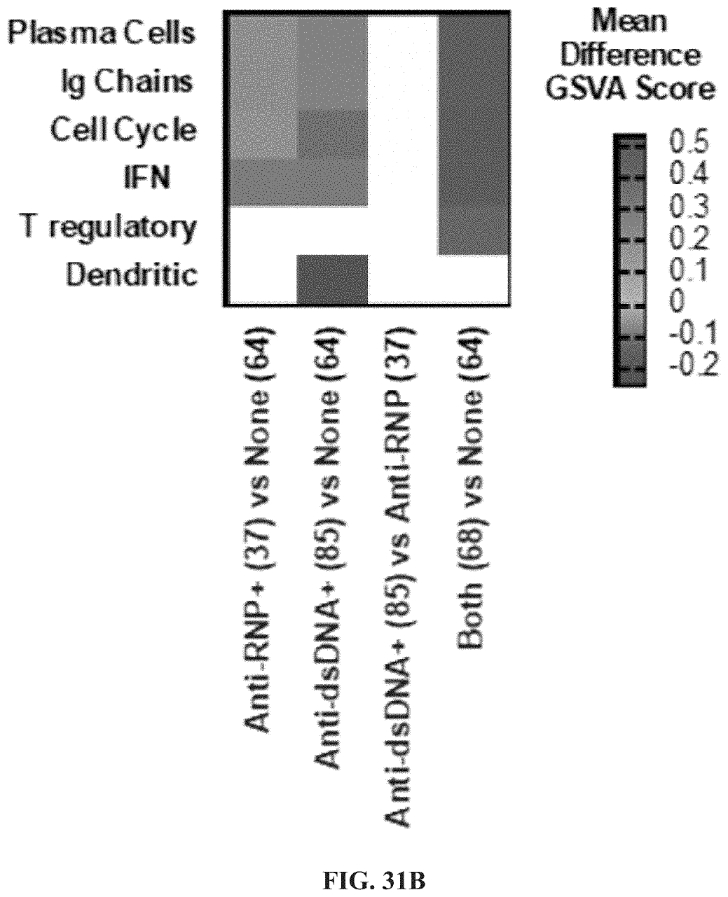

Variation in SLE disease manifestations may be a cause for cellular and gene expression heterogeneity in SLE WB. In order to determine the association between different SLE manifestations and gene expression profiles, GSVA enrichment scores for the 34 modules were compared for patients with each manifestation individually to all other manifestations. The presence of arthritis, rash, alopecia, mucosal ulcers, or vasculitis had no consistent differences on GSVA scores of the 34 modules across the ancestries. Patients of all ancestries with both anti-dsDNA and Low C had significantly higher (Sedak's multiple comparisons test, p<0.01) GSVA enrichment scores for anti-inflammation (AA. p=0.0277; EA and NAA, p<0.0001), IFN (AA, p<0.0001; EA and NAA, p<0.0001), plasma cells (AA, p=0.0032; EA and NAA, p<0.0001), immunoglobulins (AA, p=0.0044; EA and NAA, p<0.0001), monocyte cell surface (AA, p=0.03; EA, p<0.0001; NAA, p=0.04) and LDGs (AA, p=0.0008, EA p<0.0001; NAA, p=0.0103) compared to patients without anti-dsDNA and Low C. For AA and EA SLE patients, increased GSVA scores for plasma cells (AA, p=0.02; EA, p=0.0002) and Ig (AA, p=0.04; EA, p=0.0001) were also found for SLE patients with anti-dsDNA, but not Low C (FIG. 31A).

All patients in the ILL1 and ILL2 datasets were ANA positive, and 255 SLE patients also had anti-ribonucleoprotein (RNP) autoantibody titers measured. For these 255 SLE patients (19 AA, 54 NAA, and 182 EA), 86 SLE patients were positive for anti-dsDNA, 37 were positive for anti-RNP, and 68 were positive for both. Comparison of the change in gene expression profile for the anti-dsDNA, anti-RNP, or both, to the 64 patients in this subset without anti-RNP or anti-dsDNA autoantibodies showed significant increases in GSVA enrichment scores for IFN (anti-dsDNA, p=0.0023; anti-RNP, p=0.0323; both, p<0.0001), plasma cells (anti-dsDNA, p=0.01; anti-RNP and both, p<0.0001), Ig (anti-dsDNA, p=0.0039; anti-RNP and both, p<0.0001) and cell cycle (anti-dsDNA, p=0.0003; anti-RNP and both, p<0.0001). There was a significant decrease in dendritic cells for anti-dsDNA (p=0.03) and a significant increase in T regulatory GSVA scores for both (p<0.0001) (FIG. 31B).

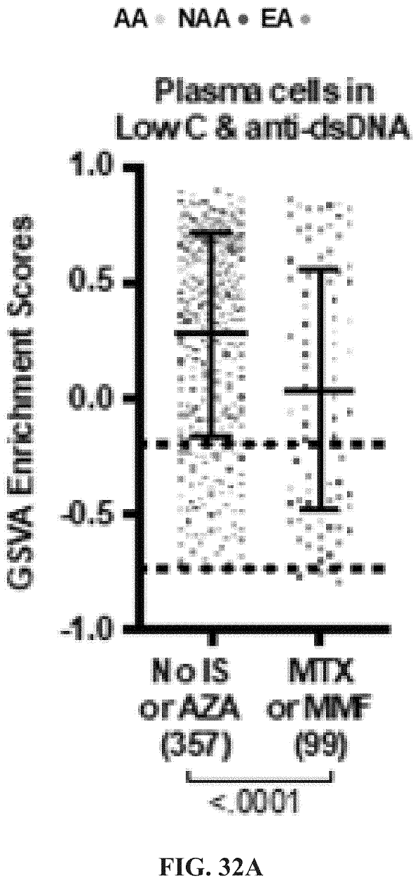

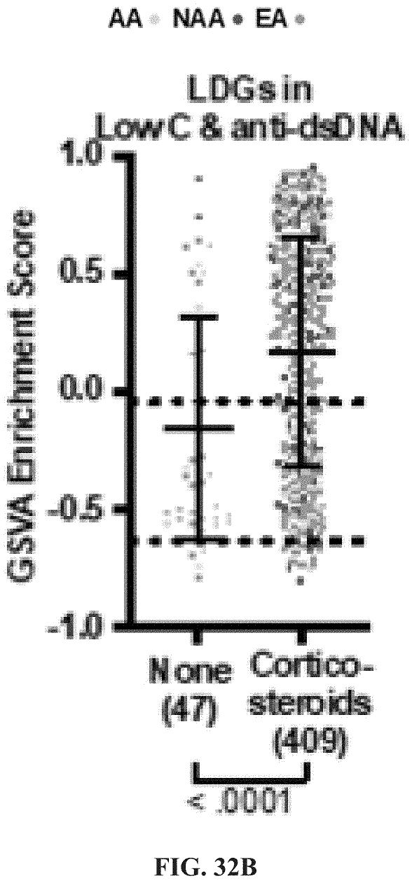

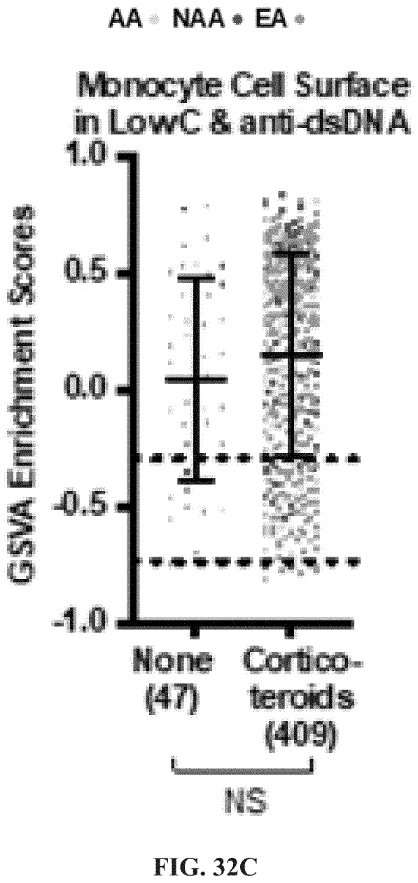

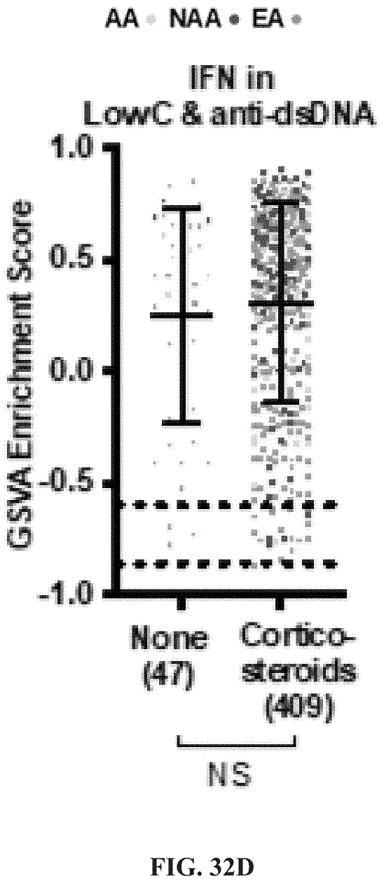

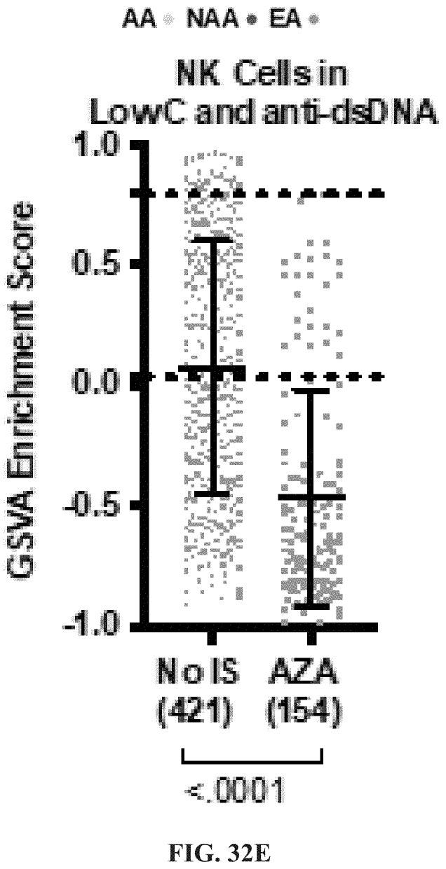

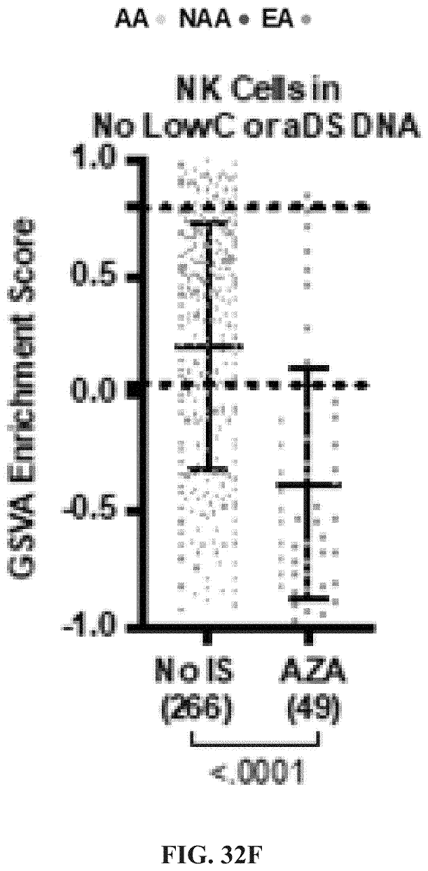

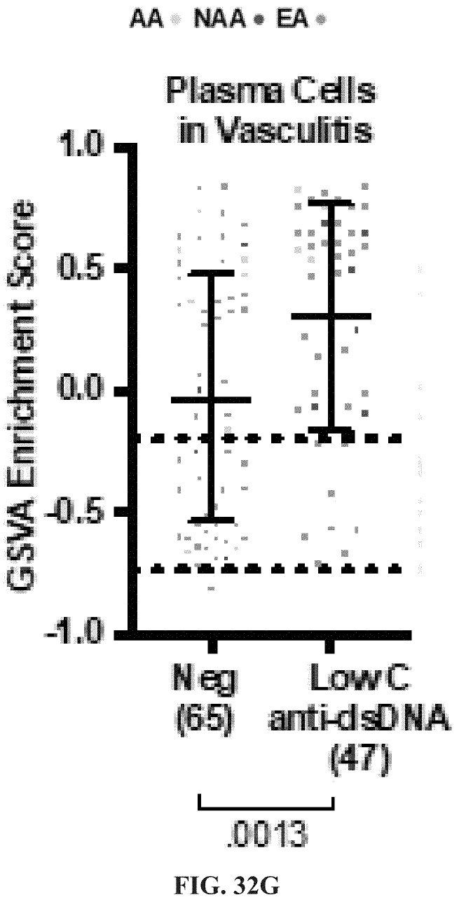

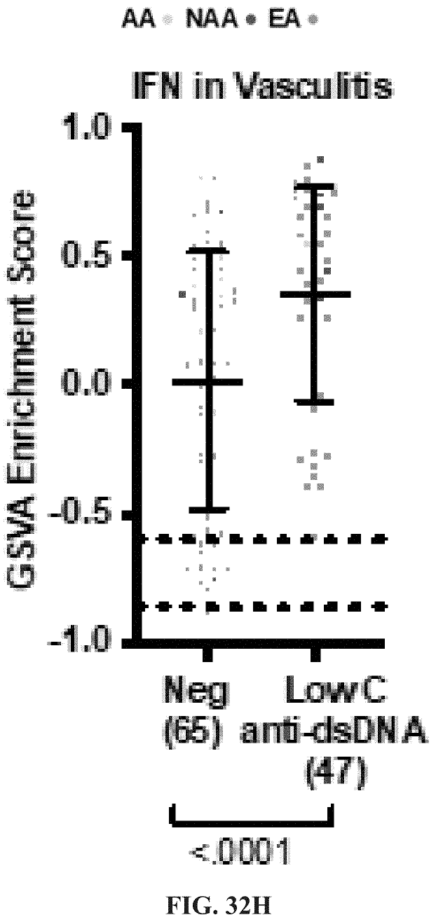

The significant increase in plasma cell signatures detected in AA patients may not be explained by AA patients having an increased incidence of anti-dsDNA and Low C; the AA patient group had the lowest number and percentage of patients with both anti-dsDNA and Low C, 23% (n=50), whereas 29% (n=320) of EA patients and 37% (n=86) of NAA patients had both anti-dsDNA and Low C. To determine whether autoantibodies and complement levels or drugs contributed more to the relationship with specific GSVA signatures, patients positive for both Low C and anti-dsDNA were compared with and without specific drugs or manifestations for cell specific GSVA scores. Patients having both Low C and anti-dsDNA had significantly lower plasma cell GSVA scores if they were also taking either MTX or MMF (FIG. 32A). 90% of patients with both Low C and anti-dsDNA were also receiving corticosteroids, and patients taking corticosteroids had significantly increased LDG GSVA scores, demonstrating that the increase in LDGs observed in patients with anti-dsDNA and Low C was related to concomitant corticosteroid usage, and not the presence of anti-dsDNA and Low C (FIG. 32B).

The increase in monocyte cell surface and IFN signature GSVA scores in patients with both Low C and anti-dsDNA was not explained by corticosteroid usage, as GSVA scores were similar between patients taking or not taking corticosteroids. The increase in IFN signature observed in EA and AA SLE patients on corticosteroids was related to the disproportionate numbers of patients with Low C and anti-dsDNA in the corticosteroid population, 39%, versus only 13% of the patients not taking corticosteroids who had both Low C and anti-dsDNA (FIGS. 32C-32D). In EA SLE patients, decreased NK cells were detected in those with anti-dsDNA or Low C. The effect was related to 23% of patients with Low C and anti-dsDNA also being on AZA (FIG. 32E) compared to only 15% of patients without low C or anti-dsDNA taking AZA (FIG. 32F) and thus not directly related to having anti-dsDNA and Low C. Vasculitis patients had a higher incidence of both anti-dsDNA and Low C, 41%, compared to 22% overall. Separation of vasculitis patients by anti-dsDNA and Low C demonstrated that the significant increase in plasma cells and IFN GSVA scores were likely related to the patients also having both anti-dsDNA and Low C, as there was a significant increase in GSVA enrichment scores for IFN and plasma cells in vasculitis patients with both anti-dsDNA and Low C (FIGS. 32G-32H; plasma cell mean difference=0.2873, p=0.0013, IFN mean difference=0.3889, p<0.0001). Thus, SLE serum components significantly contribute to individual gene expression signatures, but still may not explain the differences observed between AA, EA, and NAA patients.

Male SLE Patients Demonstrated Similar Ancestral Differences as Female SLE Patients

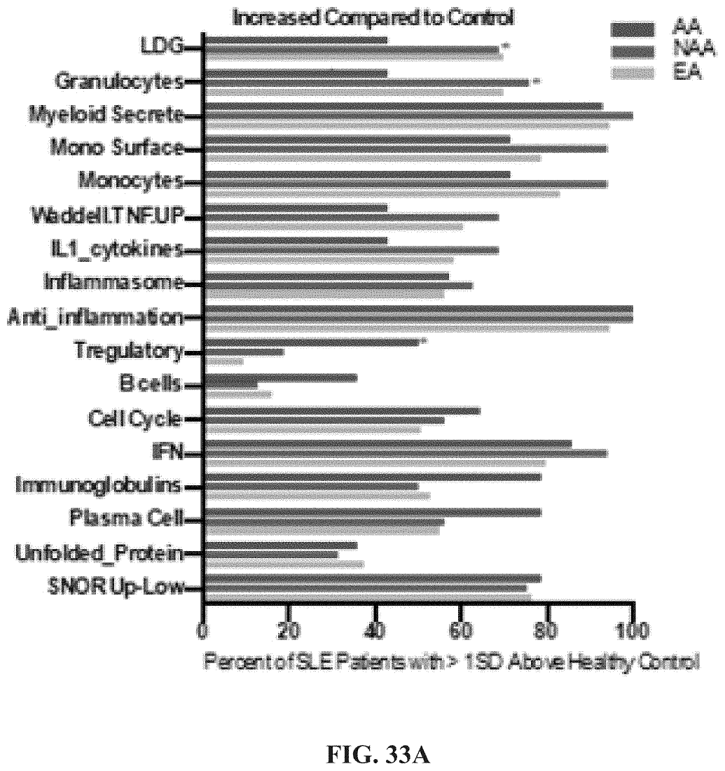

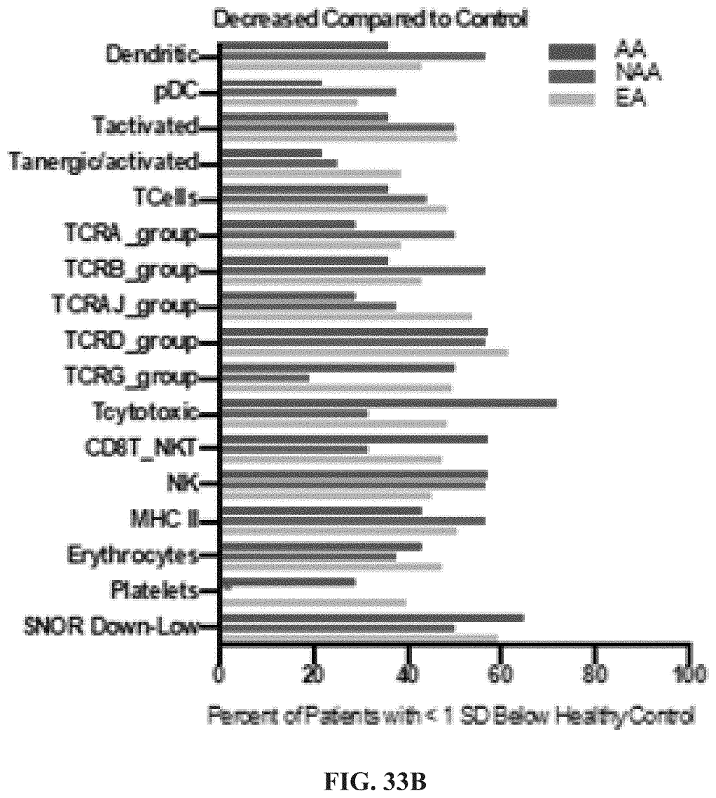

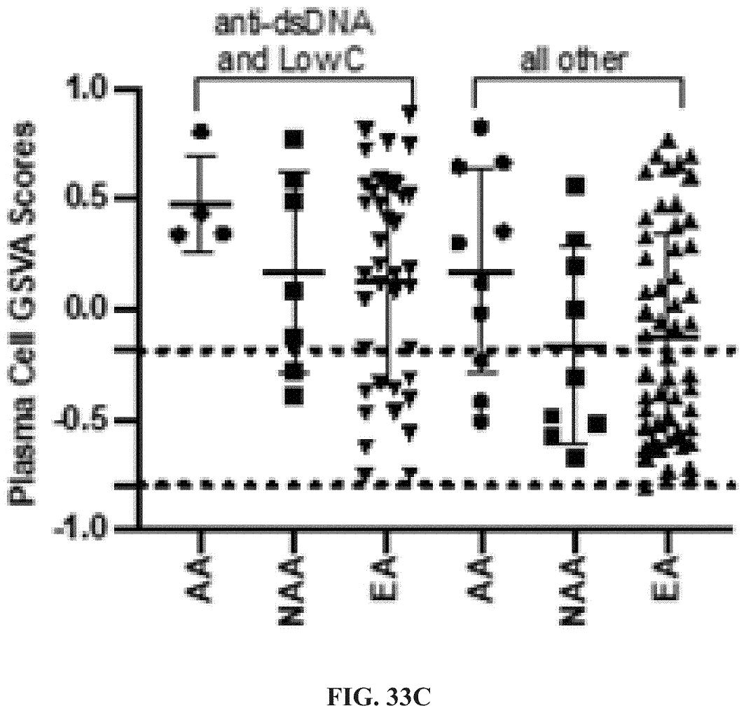

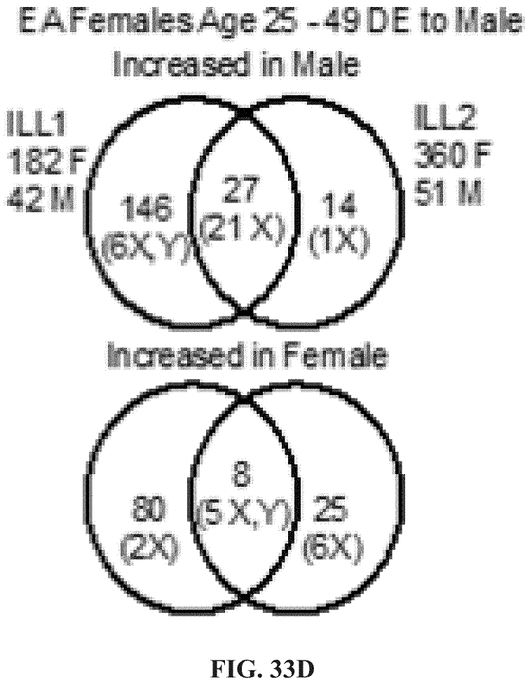

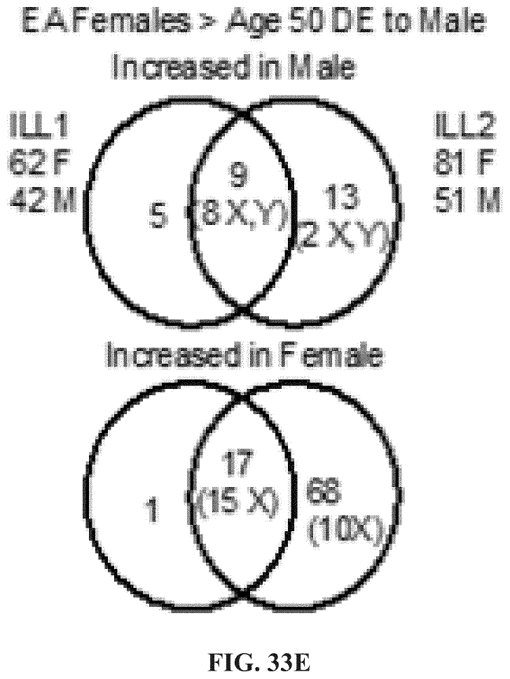

Since the frequency and severity of SLE in male and female patients with SLE is different, initially only female lupus subjects were examined. However, to determine whether ancestral differences are also observed in male lupus subjects, GSVA enrichment scores were calculated for the 34 cell and process modules for 14 AA, 93 EA, and 17 NAA GSE88884 ILL1 and ILL2 male patients and male HC. As shown in FIG. 33A, the pattern of enrichment was similar to that seen between the results obtained for female patients in FIG. 27B, with increased plasma cells, Ig, and T regulatory signatures in AA SLE patients and increased LDG and myeloid signatures in NAA and EA SLE patients. The statistical significance between the groups may not be apparent because of the low numbers of patients examined, except for the LDG and granulocyte signature in NAA compared to AA patients (p=0.0261, p=0.013), the T regulatory signature in AA compared to NAA patients (p=0.0008), and a lack of decreased platelet signatures in NAA compared to AA (p=0.0365) and EA (p=0.0001) patients. AA male patients were also less likely to have decreased TCR alpha and TCR beta signatures compared to EA (p=0.0257, p=0.0141) and NAA (p=0.0013, p=0.0017) male patients. The combination of anti-dsDNA and Low C was associated with positive plasma cell signatures, as was detected for female SLE patients (FIG. 33B).

EA SLE patients were used to determine differences between female patients and male patients with SLE. Because of the large number of female patients, the sets of female patients and male patients were able to be balanced for the percentage of patients on corticosteroids, AZA, and MTX/MMF. Further, the female patients were divided into two age groups, 25-49 years and over 50 years, because of the effects of estrogen on immune responses. For comparison of females 25-49 years old to males, there were 261 DE transcripts from the ILL1 dataset and 74 DE transcripts from the ILL2 dataset (FDR=0.05); 35 of these transcripts were in common between the two datasets, and of these, 26 were encoded on the X or Y chromosome. For comparison to females over 50 years of age, there were 32 DE transcripts from ILL1 and 97 DE transcripts from ILL2; 26 of these transcripts were in common between the two datasets, and of these, 23 were encoded on the X or Y chromosome (FIGS. 33C-33E). For comparison of females age 25-49, there were several increased TCR alpha J region chains, but no increased expression of previously reported estrogen induced genes. There were no DE genes associated with plasma cells or interferon signatures. There were a few transcripts associated with granulocytes (CSF2RA, CEACAM8, DEFA4, CLEC4D, BPI) increased in ILL2 males compared to females over age 50 and ILL1 males compared to females 25-49 years, but no consistent pattern based on age of the female patients.

Ancestry Provides the Gene Expression Backbone, but SOC Drugs Greatly Modify Gene Expression

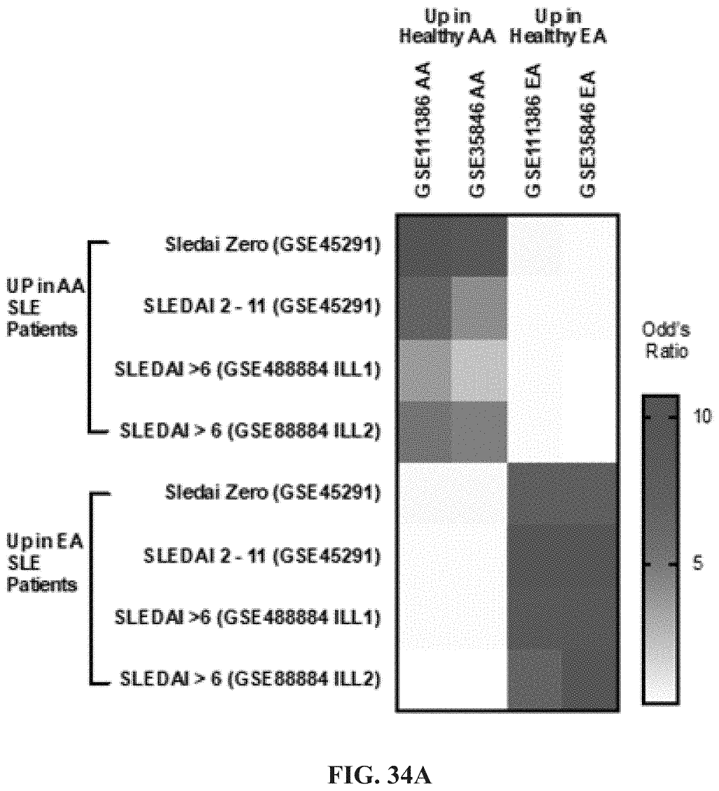

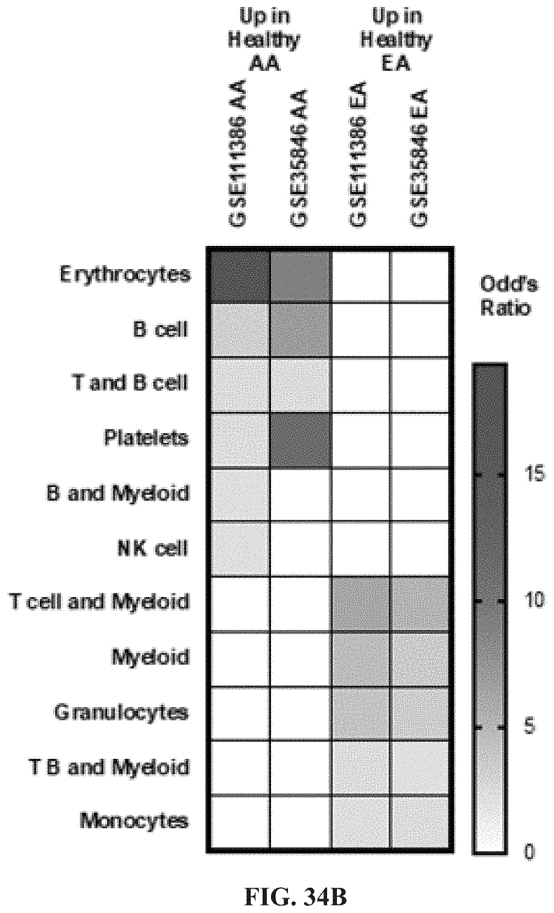

Analyses of the DE transcripts between different ancestries have shown that EA and NAA populations overexpressed the Duffy blood group antigen ACKR1, the platelet and monocyte receptor CD36, and G6PD, in comparison to all AA populations, and that all of these genes have risk alleles resulting in decreased expression in the AA population. Therefore, gene expression differences detected between SLE patients was shown to be related to heritable differences manifesting in expressed genes in hematopoietic cells of healthy subjects of different ancestries. In order to demonstrate this, gene expression analysis of adult, self-described AA and EA HC subjects was carried out on two separate microarray datasets of normal subjects of different ancestries. Both datasets had hundreds of DE transcripts for healthy AA patients compared to healthy EA patients; GSE111386 (10 AA, 57 EA) had 3,295 DE transcripts and GSE35846 (22 AA, 55 EA) had 2,476 DE transcripts (FDR of 0.2) with 1,234 transcripts in common between the two datasets. Significant odds ratios (overlap p value<0.0001) were documented between transcripts increased in HC AA subjects compared to HC EA subjects, and transcripts increased in AA SLE patients compared to EA SLE patients in all four SLE datasets: GSE88884 ILL1, GSE88884 ILL2, GSE45291 with SLEDAI of 0, and GSE45291 with SLEDAI of 2-11) and significant odds ratios (Fisher's exact p value<0.0001) were demonstrated between transcripts increased in EA HC subjects and those increased in EA SLE patients, but no significant overlap was observed between AA HC subjects and EA SLE patients, or between EA HC subjects and AA SLE patients (FIG. 34A).

I-scope analysis of the transcripts increased in healthy AA patients demonstrated an increase in B cell, dendritic, erythrocyte, and platelet associated transcripts compared to EA HC subjects, and an increase in granulocyte, monocyte, and myeloid transcripts in healthy EA subjects compared to AA HC subjects (FIG. 34B). IFI27, a gene commonly used to monitor the IFN signature, was increased in healthy AA subjects in both datasets, and IFITM2, another IFN signature gene, was increased in both healthy EA datasets. CXCLS, IL32, and TNFSF4 were increased in healthy AA subjects in both datasets, and CXCL8, CXCL1, GRN, MMP9, TNFSF14, and CXCL6 were increased in healthy EA subjects in both datasets. There were no genes associated with plasma cells or LDGs DE between AA and EA HC subjects, and the majority of the IFN signature genes and inflammatory secreted genes were not differentially expressed between AA and EA subjects, including IFI44, IFI44L, C1QA, C1QB, C1QC, CCL2, CXCL10, CXCL2, IL1B, TNF, and THBD.

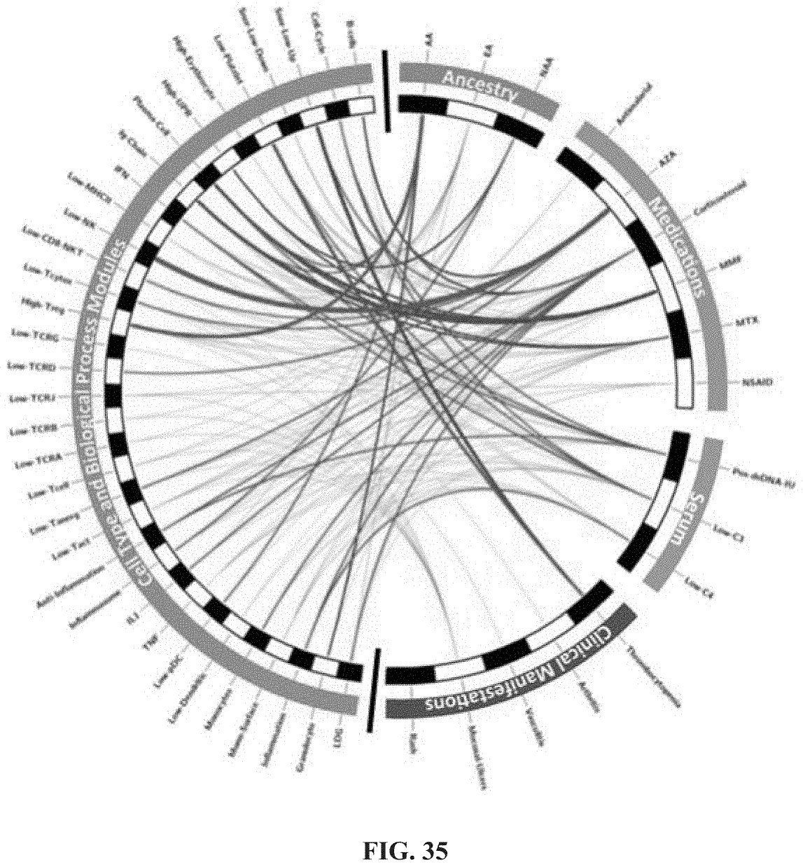

In order to determine the relative importance of ancestry, SOC drugs, and SLE manifestations to gene expression signatures, stepwise logistic regression analysis was performed for each of the 34 cell type and process signatures using the variables of ancestry (AA, EA, NAA), SOC drugs (MTX, MMF, AZA, corticosteroid drugs, NSAID drugs, and anti-malarial drugs), SLE serum components (anti-dsDNA, Low C3, Low C4) and SLE manifestations (arthritis, rash, mucosal ulcers, vasculitis, thrombocytopenia). FIG. 35 shows a CIRCOS visualization of the odds ratios for each variable significantly (p<0.05) contributing to each GSVA enrichment score. Ancestry significantly influenced 21 of the 34 cell type and process module scores. For AA patients, there was a negative relationship to LDG, granulocytes, IL1 cytokines, and inflammasome and a positive relationship to low pDC, Treg, IFN, plasma cells, Ig, and B cells. Low MHC II and the low SNOR up were negatively associated with NAA patients, and NAA status was positively associated with inflammasome, low T cells, and platelets. For EA patients, there was a negative association to low NK cells, granulocytes, UPR, low SNOR down, and the cell cycle and a positive association to the inflammasome, low platelets, and Treg. SLE serum components significantly influenced 19 of the 34 modules with the most significant odds ratios and confidence intervals for the IFN signature, cell cycle, plasma cells, and Ig. SLE manifestations influenced the transcriptome the least, with significant relationships to 14 signatures, but with confidence intervals very close to 1. SOC drugs influenced every cell and process module GSVA enrichment score, with the most profound effects by AZA on NK and B cells, MTX/MMF on plasma cells, Ig, and T cells, and corticosteroids on myeloid cells (based on Spearman correlation coefficients between variables, confidence intervals, p values, and odd's ratios).

Based on this data, it was hypothesized that balancing SOC drugs in SLE patients may significantly reduce the number of DE transcripts between AA and EA SLE patients. The DE analysis was repeated on GSE88884 ILL1 and ILL2 AA to EA SLE patients from FIGS. 26A-26D, but this time with selected AA and EA SLE patients of similar daily steroid usage (mean, median, and mode), no immunosuppressive drugs, and similar percentages receiving anti-malarial drugs and NSAID drugs. There were 606 DE transcripts from the ILL1 dataset AA (n=41) to EA (n=144), and 535 DE transcripts for ILL2 dataset AA (n=44) to EA (n=154) (FDR=0.05); a loss of 83 and percent 82 percent of the DE transcripts, respectively, compared to DE analysis of all ILL1 and ILL2 AA to EA SLE patients with non-matched SOC drugs in FIGS. 26A-26D. Thus, the combination of different drug regimens and ancestry significantly changed patient gene expression having profound implications for interpretation of gene expression analyses.

Discussion

The analysis and results herein provide a significant understanding of the contributions of SLE patient ancestry and SOC drugs to the subject's gene expression profile. Furthermore, the results demonstrate important ancestry-based gene expression differences present in healthy controls of AA, NAA, and EA ancestry, that serve as the background for the heterogenous transcriptomic signatures detected in SLE patients. Thousands of DE transcripts were identified when AA, EA, and NAA SLE patients were compared to each other. There were no detectable transcripts when SLE patients of the same ancestry were randomized and compared, demonstrating that the differential expression between ancestral groups was determined by genetic ancestral make-up to a significant extent.

The ancestry-related differences in gene expression profiles highlights an important issue of using appropriate numbers of controls with matching ancestry to determine meaningful changes in a disease state. A striking overlap was observed between unrelated AA HC subjects and EA DE analyses and the separate AA SLE and EA DE analyses of 1,810 patients. Somewhat surprisingly, the AA HC subjects overlapped with AA SLE patients better than the EA HC subjects to EA SLE patients, since the AA subjects may be expected to contain more admixture than the EA subjects. These data demonstrate that ancestral gene expression differences serve as a backdrop on which the transcriptomic signature is built and accounts for much of the heterogeneity in blood gene signatures. Ancestral SNPs in HC may be estimated to account for about 17-28% of variation in gene expression, and these results demonstrated these gene expression differences readily contribute to an SLE patient's transcriptomic signature. Additionally, several ancestral-related genes divergent between AA and EA populations that are also involved in immune responses were differentially expressed between SLE patients and HC subjects of different ancestries: IL8, CXCL1, CXCL5, STAT1, CEPBP, ITGAM, and CD58, demonstrating that ancestral SNPs contribute to the gene expression profile. It may be shown that AA is associated with increased responses to infection and increased expression of inflammatory response genes. While generally, an increased inflammatory response may be associated with an increase in innate immune response cells, the results actually showed a depletion, or less of an increase, in myeloid cells in AA patients compared to EA and NAA patients. Interestingly, there was no significant difference in expression of transcripts for inflammatory mediators such as complement, TNF, and CXCL10, despite the difference in detection of cell types that generally produce these inflammatory mediators. This result indicates that individual innate immune cells from AA patients produce more inflammatory mediators.

The ramifications of these results toward interpretation of gene expression analysis are important. HC of AA and EA ancestries were reproducibly shown to be disparate in transcripts for erythrocyte, platelet, B cell, T cell, NK cell, granulocytes, and monocyte transcripts; furthermore, this transcript data agrees with cell counts and genetic differences between ancestries. Platelet counts may be shown to be higher in AA than EA patients, and the Duffy Null Polymorphism (ACRK1 gene) may be shown to be a cause of decreased neutrophil counts in AA patients. CD19+ B cell counts may be shown to be increased in AA patients compared to EA patients, and CD3+ T cells may be shown to be increased in EA patients versus AA patients, although overall lymphocyte counts may not be different. The erythrocyte transcripts increased in AA patients may be related to increased reticulocytes in the circulation, and this may be explained by AA patients more frequently possessing x-linked G6PD alleles responsible for the African ancestry-associated G6PD deficiency prominent in AA males. Reticulocytosis may be augmented in AA patients with SLE, as persons with G6PD deficiency may have induced hemolysis secondary to infection and leukocyte phagocytosis. G6PD was decreased in both AA SLE patients and AA HC subjects compared to EA SLE patients and EA HC subjects. The ancestral transcriptomic backbone may be emphasized depending on HC comparators, and as a result, many DE transcripts may be inappropriately attributed to the disease instead of the ancestry, whether or not the allelic differences play an actual role in the pathogenesis of SLE. Analysis of purified cell types from AA and EA SLE patients may show only about 10% similar transcripts, indicating disparate constitutive pathways and metabolism operating in AA and EA SLE patient hematopoietic cells. Although these data and results described herein confirmed strong ancestral contributions to the SLE signature, there were patients within all ancestries with disparate signatures from the prevailing ancestral type, demonstrating that personalized medicine strategies to determine the type of lupus may be helpful, instead of relying on ancestral background or group statistics (e.g., median or mean). Additionally, drugs and their effect on cell populations and signaling pathways may be taken into account to help focus attention onto pathways and cells involved in disease and not the treatment. The IL-1, inflammasome, and LDG increased signatures detected in NAA patients appeared to be related to corticosteroid drugs. This signature may be further deciphered by performing studies of healthy NAA patients. Single-cell technology may be used to elucidate and observe effects of ancestry and SOC drugs, and to distinguish between out cell populations prominent in ancestries and induced or repressed by concomitant drugs, from cell populations actively participating in disease processes.

The results demonstrate a strong relationship between SLE serum components and circulating Ig, plasma cell, cell cycle, and IFN GSVA scores; further, this association was more pronounced in EA and NAA patients than AA patients. These data also and demonstrated that observed increases in plasma cell signatures in pediatric AA SLE patients are likely related to ancestry, and not disease activity. Increased Ig production is associated with plasma cells, and Ig genes have been used as a proxy for plasma cell measurements in microarray datasets. Both healthy control AA and EA datasets were on Illuminate chips that harbor only a few Ig genes, so although Ig genes were not detected as different between healthy AA and EA, in some cases, this signature may derive from healthy B cells, which may explain why AA plasma cell GSVA scores did not correlate as well with serum component measurements. Single-cell RNAseq analysis of isolated hematopoietic cell types in healthy subjects may demonstrate that B cells have increased Ig transcripts compared to all cell types except plasma cells. Lupus in the AA population may be strongly biased towards generation of plasma cells. Since healthy AA subjects, in two separate datasets, also showed increased transcripts associated with B cells, the increase in plasma cells may have an origin in the inherent differences in the healthy AA population.

Further, the results herein demonstrated that increased IFN signatures were associated with anti-dsDNA and Low C in all ancestry groups. AA SLE patients may be shown to be more likely to have an IFN signature than EA SLE patients; the results obtained also detected significantly more AA than EA SLE patients with an IFN signature, but the percentages of IFN-positive patients were greater than 75% for both ancestry groups and less useful for distinguishing AA from EA SLE patients. Corticosteroids may be demonstrated to decrease IFN signaling, but this effect was not seen in this study and may be a result of the large number of patients on corticosteroids also having both anti-dsDNA and Low C. In some cases, monocytes appear to retain the IFN signature in inactive lupus patients, confounding usage of this signature to determine disease activity, and the increased IFN signature in SLE patients with anti-dsDNA and Low C may be accompanied with increased signatures for monocyte cell surface transcripts.

Besides the effect of ancestry and SLE serum components, the results and data demonstrated the profound effect SOC therapies have on SLE patient gene expression profiles, and indicate a method of accounting for these effects using the change in GSVA enrichment score associated with drug administration. When the SOC drugs were matched between AA and EA SLE patients, more than 80% of the DE transcripts were lost between AA and EA SLE patients from ILL1, and this was repeated in ILL2. Patients with increased GSVA scores compared to controls for the inflammasome, IL-1, and myeloid signatures were significantly increased in the NAA population, and the number of DE transcripts between AA and EA patients was almost twice the difference between AA and EA patients, indicating at first that this population was the most different from AA and EA patients. However, further analysis determined that NAA were also receiving more corticosteroids and immunosuppressive therapy, and that this therapy was likely accounting for much of their increased myeloid and decreased lymphocyte signatures.

Further, the results showed increased signatures for myeloid cells in pediatric EA and NAA (Hispanic) SLE compared to AA patients, although this difference may be related to the benign neutropenia common in people of African ancestry, the increased corticosteroids taken by NAA patients, and not lupus related. By using more than 1,500 SLE patients, it was shown that AA SLE patients did not have significantly enriched plasma cell signatures compared to EA and NAA ancestry groups, if all patients had both anti-dsDNA and Low C, or if all patients were receiving MTX or MMF. Although AA patients also had the lowest number of patients on AZA, and AZA therapy was related to decreased B cell GSVA scores, there were not enough patients receiving this therapy for this drug to account for the differences noted between ancestry groups. In confirmation of the methodology used, AZA treatment significantly decreased NK cell GSVA scores in all three ancestry groups in the GSE88884 and GSE45291 datasets, consistent with an effect of AZA on NK cells. EA patients had significantly higher NK cell GSVA scores compared to NAA patients, when both were not receiving AZA treatment; however, there was no significant difference when both ancestry groups were receiving AZA treatment.

The association of neutrophil granule protein transcripts (LDG signature) with corticosteroid usage may be observed. Corticosteroid usage also had a significant effect on most myeloid signatures including monocyte cell surface transcripts, myeloid secreted protein transcripts, and IL1 transcripts. This may be a result of increasing this population in the periphery as steroids may be shown to increase demargination of mature neutrophils. The LDG signature was also prominently detected in EA SLE patients with SLEDAI values of zero on corticosteroids. LDGs in autoimmunity may be described as being inflammatory and contributing to SLE pathogenesis from data obtained from in vitro experiments demonstrating an increased capacity for production of inflammatory cytokines. However, corticosteroids may be demonstrated to induce human monocytes to secrete G-CSF, and G-CSF may mobilize neutrophils from the bone marrow, indicating a mechanism where chronic corticosteroid use may promote the release of immature neutrophils. G-CSF therapy for neutropenia in lupus patients may induce flares and vasculitis, indicating a pathologic role for G-CSF. G-CSF also may be shown to increase a glycosylated, membrane form of MPO on mature neutrophils and monocytes, and this form of MPO may bind to E-selectin on human endothelium and induce cytotoxicity. The strong relationship between LDGs and corticosteroid usage, and yet the presence of transcripts for granule proteins in patients reportedly not taking corticosteroids, may be indicative that there may be two or more different populations of granule expressing cell populations. The relative contribution to microarray signatures of genes related to neutrophils may be disparate between AA and other populations and may not reflect differences in lupus. Therefore, different neutrophil signatures may arise because of ancestry-related rather than lupus-related differences.

The observed lack of difference in GSVA scores for inflammatory cell populations, inflammatory cytokines, IFN signatures, and the TNF pathway for patients treated with anti-malarial drugs (e.g., hydroxychloroquine (Plaquenil), chloroquine (Aralen), and quinacrine (Atabrine)) compared to all other treatments was surprising, as chloroquine may decrease anti-inflammatory cytokine production. Experiments may demonstrate that hydroxychloroquine blocks TLR 9/7 stimulation and subsequent IFN production in vitro. As plasmacytoid dendritic cells were generally decreased in the periphery of SLE patients, perhaps the target cells for anti-malarial drugs are found in tissues, but this data demonstrated no significant changes in cell populations or processes associated with anti-malarial usage in the periphery. Surprisingly, NSAID drugs had more of an effect on gene expression profiles than anti-malarial drugs. Although commonly known as cyclooxygenase isoenzyme inhibitors, NSAID drugs may be shown to block caspases and inflammation; although the change in GSVA score was not greater than 0.2, there did appear to be a decrease in LDGs and the anti-inflammation signature, at least in EA SLE patients.

Major differences may be reported in lupus cohorts between male and female SLE patients with respect to renal involvement and serological manifestations. While renal patients were excluded from the ILL1 and ILL2 clinical trials, among patients with non-renal manifestations, there did not appear to be consistent differences in gene expression other than the expected transcripts encoded on the X and Y chromosomes. Gene expression differences attributable to estrogen in female patients under 50 may be expected; however, analysis of the DE transcripts did not reveal an obvious link to effects on the immune system. The ancestral differences between males also appeared similar to the ancestral differences between females, indicating the ancestral component to gene expression are more important to take into consideration than male-vs.-female differences.

Self-identified ancestry gave useful information for the genetic background of an individual; further, pairing studies with genetic data may be performed to determine specific ancestry admixtures. The current results provide a framework for determining the meaningful contributions to the SLE disease transcriptome and to separate these contributions from the effects of SOC therapy and ancestry.

In summary, ancestry plays an important role in the gene expression profiles of individual SLE patients and by implication contributes to the molecular pathways operative in each subject. Understanding, for example, that some self-described AA patients may have higher levels of transcripts for B cells, erythrocytes, and platelets compared to EA SLE patients may help explain differences in gene expression data that do not manifest from the SLE disease, but from the patient's ancestral background. The relationship of corticosteroid drugs to LDGs has implications against using this signature as a measure of disease severity or interpreting LDGs as playing a role in worsening disease, as worsening disease may prompt an increase in corticosteroid doses. Combinations of different ancestry, SOC therapy, and autoantibody production associated with gene expression profiles m datasets comprised of different populations from around the world difficult to compare. Understanding the contributions of the gene expression signature components may permit a better understanding and interpretation of the signatures and their relationship to disease status.

Methods

Gene expression datasets were obtained as follows. Data were derived from publicly available datasets on Gene Expression Omnibus (GEO, www.ncbi.nlm.nih.gov/geo/). Raw data sources were used as follows: GSE88884 female whole blood Illuminate 1 (ILL1; 10 female HC, 798 female SLE (540 EA, 101 AA, and 157 NAA); all with SLEDAI≥6), GSE88884 female whole blood Illuminate 2 (ILL1; 7 female HC, 767 female SLE (577 EA, 115 AA, and 75 NAA) all with SLEDAI≥6), GSE88884 male whole blood Illuminate 1 SLE (ILL1: 5 male HC, 59 male SLE (6 AA, 42 EA, and 11 NAA), GSE88884 male whole blood Illuminate 2 (ILL2: 4 male HC, 65 male SLE (8 AA, 51 EA, and 6 NAA); (GSE45291 whole blood (9 female HC, female SLE: 73 AA, 71 EA with SLEDAI of 2-11), GSE45291 whole blood (9 female HC, female SLE: 25 AA, 75 EA; all with SLEDAI of 0), GSE35846 whole blood from healthy females (55 EA, 22 AA), and GSE111386 whole blood from healthy females (10 AA, 57 EA). Clinical data including disease activity assessed by SLEDAI, anti-dsDNA titers, complement levels, disease manifestations, and standard of care drugs were provided by Eli Lilly (GSE88884 Illuminate I and Illuminate 2).

Quality control and normalization of raw data files were performed as follows. Statistical analysis was conducted using R and relevant Bioconductor packages. For datasets GSE88884 and GSE45291, non-normalized arrays were inspected for visual artifacts or poor RNA hybridization using Affy QC plots. To increase the probability of identifying differentially expressed genes (DEGs), analysis was conducted using normalized datasets prepared using both the native Affy chip definition files, followed by custom Brain Array Entrez CDFs maintained by the University of Michigan Molecular and Behavioral Neuroscience Institute. The Affy CDFs include multiple probes per gene and almost twice as many probes as BA CDFs. Whereas Affy chip definition files can provide the greatest amount of variance information for Bayesian fitting, the Brain Array chip definition files are used to exclude probes with known non-specific binding and those shown by quarterly BLASTs to no longer fall within the target gene. Illumina CDFs were used for the Illumina datasets (GSE35846, GSE111386).

Differential gene expression (DE) analysis was performed as follows. GCRMA normalized expression values were variance-corrected using local empirical Bayesian shrinkage, followed by calculation of DE using the ebayes function in the open source BioConductor LIMMA package (www.bioconductor.org/packages/release/bioc/html/limma.html). Resulting p-values were adjusted for multiple hypothesis testing and filtered to retain DE probes with a False Discovery Rate (FDR) of less than 0.05.

Determination of female and male controls was performed as follows. Log 2 expression values were used to determine sex of unknown healthy controls and to compute sex module scores using the formula below:

Sex module=XIST log 2expression+TSIX log 2expression−(UTY log 2expression+USP9Y log 2expression).

Female controls scored above zero and male controls scored below zero.

I-Scope

I-scope is a tool developed to identify immune infiltrates. I-scope was created through an iterative search of more than 17,000 genes identified in more than 50 microarray datasets. From this search, 1,226 candidate genes were identified and researched for restriction in hematopoietic cells as determined by the HPA, GTEx, and FANTOMS datasets (www.proteinatlas.org). A set of 926 genes met a set of criteria for being mainly restricted to hematopoietic lineages (brain, reproductive organ exclusions were permitted). These genes were researched for immune cell specific expression in hematopoietic sub-categories: T cells, Regulatory T Cells (Treg), Activated Tcells (Tactivated), Anergic/Activated cells (Tanergic), Alpha/Beta T cells (abTcells), Gamma delta T cells (gdTcells), CD8 T, NK/NKT cells, NK cells, T or B cells, B cells, B or pDC cells, GC B cells, T or B or Myeloid cells, B or Myeloid cells, Antigen Presenting Cells or MHC Class II expressing cells (MHC II), Dendritic cells (Dendritic), Plasmacytoid dendritic cells (pDC), Myeloid cells (Myeloid), Monocytes, Plasma Cells (Plasma), Erythrocytes (Erythro), Granulocytes (Neut), Low density granulocytes (LDG), and Platelets. Transcripts are entered into I-scope, and the number of transcripts in each category were determined. Odds ratios were calculated with confidence intervals using the Fisher's exact test in R.

Gene ontology (GO) biological pathways were determined as follows. The database for annotation, visualization and integrated discovery (DAVID) (david.abcc.ncifcrf.gov/) was used to determine enriched GO biological pathways.

Gene Set Variation Analysis (GSVA) was performed as follows. The GSVA (V1.25.0) software package is an open source package available from R/Bioconductor, and was used as a non-parametric, unsupervised method for estimating the variation of pre-defined gene sets in patient and control samples of microarray expression data sets (www.bioconductor.org/packages/release/bioc/html/GSVA.html). The inputs for the GSVA algorithm were a gene expression matrix of log 2 microarray expression values (Brain Array chip definitions) for pre-defined gene sets co-expressed in SLE datasets. Enrichment scores (GSVA scores) were calculated non-parametrically using a Kolmogorov Smirnoff (KS)-like random walk statistic and a negative value for a particular sample and gene set, meaning that the gene set has a lower expression than the same gene set with a positive value. The enrichment scores (ES) were the largest positive and negative random walk deviations from zero, respectively, for a particular sample and gene set. The positive and negative ES for a particular gene set depend on the expression levels of the genes that form the pre-defined gene set.

Enrichment modules containing cell type and process-specific genes were created through an iterative process of identifying DE transcripts pertaining to a restricted profile of hematopoietic cells in 13 SLE microarray datasets, and checked for expression in purified T cells, B cells, and Monocytes to remove transcripts indicative of multiple cell types. Genes were identified through literature mining, GO biological pathways, and STRING interactome analysis as belonging to specific categories. The Low Disease (Signature) Up and Low Disease (Signature) Down are the seven most over-expressed and seven most under-expressed transcripts by log fold change for 348 female patients from dataset GSE88884 (ILL1 and ILL2) that were not separated from healthy controls by principal component analysis (PCA) compared by limma DE analysis to HC (FDR=0.05). The LDG signature was taken from purified LDGs DE to HC and SLE neutrophils, (Villaneueva, 2011) and consists mainly of neutrophil granule proteins from Module B as described in Kegerreis et al (2019). The overlap in genes between some signatures was intentional and used to check that signatures were behaving cohesively between patients.

Weighted Gene Coexpression Network Analysis (WGCNA) was performed as follows. WGCNA is an open source package for R available at horvath.genetics.ucla.edu/html/CoexpressionNetwork/Rpackages/WGCNA/.

Log 2 normalized microarray expression values for the GSE88884 ILL1 and ILL2 datasets were filtered using an IQR to remove saturated probes with low variability between samples and used as inputs to WGCNA (V1.51). Adjacency co-expression matrices for all probes in a given set were calculated by Pearson's correlation using signed network type specific formulae. Blockwise network construction was performed using soft threshold power values that were manually selected and specific to each dataset in order to preserve maximal scale free topology of the networks.

Resultant dendrograms of correlation networks were trimmed to isolate individual modular groups of probes, labeled using semi-random color assignments, based on a detection cut height of 1, with a merging cut height of 0.2, with the additional use of a partitioning around medoids function. Final membership of probes representing the same gene into modules was based on selection of greatest scale within module correlation against module eigengene (ME) values. Correlation to ancestry was performed using Pearson's r against MEs, defining modules as either positively or negatively correlated with those traits as a whole.

Gene Overlap analysis was performed as follows. Gene Overlap is an R bioconductor package (www.bioconductor.org/packages/release/bioc/html/GeneOverlap.html), which was used to test the significance of overlap between two sets of gene lists. It uses the Fisher's exact test to compute both an odd's ratio and overlap p value. For comparison of datasets on different array platforms (Illuminate versus Affymetrix), an FDR of 0.2 was used.

Logistic regression modeling was performed as follows. SAS 9.4 (Cary, N.C.) was used for stepwise logistic regression. GSVA enrichment scores greater or less than healthy control averages plus or minus one standard deviation were determined, and SLE patients were assigned a 1 or 0 based on having a signature greater than or less (Low) than HC, respectively. These scores were used as 34 dependent binary variables to be modeled individually as the outcome variable to 17 independent categorical (e.g., binary) variables, including ancestry (AA, EA, and NAA), drugs (corticosteroid drugs, antimalarial drugs, NSAID drugs, Azathioprine, Methotrexate, Mycophenalate mofetil), and SLE manifestations (rash, arthritis, mucosal ulcers, vasculitis, thrombocytopenia, anti-ds DNA, Low C3, and Low C4). Spearman correlation coefficients were determined between variables, followed by stepwise linear regression, in order to determine if groups were too similar to give independent information to the model. Further, odd's ratios, p values, and confidence intervals were determined Immunosuppressive as a general category was removed since it had a Spearman correlation greater than 0.4 compared to MTX and MMF. The stepwise approach was used to produce the statistically significant model. The results of any model that violated the Hosmer Lemeshow test were discarded.

CIRCOS analysis was performed as follows. CIRCOS (V0.69.3) software was used to visualize the odd's ratios determined by stepwise logistic regression analysis. Odd's ratio values are non-negative, and a change from an odds ratio of 0.5 to 0.25 is the same relative change as that between 2.0 and 4.0. For representative visualization, odd's ratios between 0 and 1 were converted to the 1/X value, where X is an odd's ratio between 0 and 1.

Statistical analysis was performed as follows. GraphPad PRISM 7 version 7.0c was used to calculate or perform mean, median, mode, standard deviation, ANOVA, Tukey's multiple comparisons test, Sedak's multiple comparisons test, linear regression analysis, and unpaired t-test with Welch's correction. The Fisher's exact test was performed in R.

Data availability was as follows. All microarray datasets in this publication are available on the NCBI's database Gene Expression Omnibus (GEO) (www.ncbi.nlm.nih.gov/geo/).

Code availability was as follows. All software used to produce results described in this example is open source, and freely available for R. Additionally, example code used to produce results described in this example for LIMMA, GSVA and WGCNA are available at figshare (www.figshare.com). File names are “AMPEL BioSolutions LIMMA Differential Expression Analysis Code”, “AMPEL BioSolutions Gene Set Variation Analysis Code”, and “AMPEL BioSolutions Weighted Correlation Network Analysis WGCNA Code”.

### Example 7: Ancestry Influences the Gene Expression Profile in Systemic Lupus Erythematosus (SLE) and Contributes to Gene Expression Heterogeneity in Lupus Patients

Systemic Lupus Erythematosus (SLE) is a complex autoimmune disease with both sex and ancestral bias. Gene expression analysis has revealed complex heterogeneity between SLE patients, making deconvolution of the data difficult and delineation of the impact of different disease drivers uncertain. Therefore, the individual contributions of ancestry, gender, and medications to gene expression heterogeneity were assessed. Further, the association of gene expression profiles with various SLE manifestations was determined.

Bulk Differential Expression (DE) analysis and Gene Set Variation Analysis (GSVA) were carried out on 1,903 SLE patients of African (AA), European (EA), and Native American (NAA) ancestry. Modules of genes defined by co-expression in patients and representing either functional or cell specific groups were used to determine the relationship between drugs, SLE manifestations and individual patient gene expression. Logistic regression analysis was used to understand the relative contribution of ancestry, drugs and SLE manifestations to gene expression signatures.

Gene expression analysis between female disease-matched SLE patients of AA, EA, and NAA ancestry revealed thousands of DE transcripts between ancestries, but none within a single ancestry. AA, EA, and NAA SLE patients had significantly different cellular contributions to gene expression, and these differences were related to significantly different percentages of patients in each ancestry with specific signatures. GSVA showed an increase in plasma cells, B cells, and T cells in the majority of AA SLE patients, and an increase in myeloid cells in most EA and NAA SLE patients. Corticosteroid drugs and immunosuppressive drugs significantly changed gene expression and contributed to the disparate signatures between and within ancestry groups. Anti-dsDNA autoantibodies and low complement, but not other clinical features of SLE, were significantly associated with gene expression in AA, EA, and NAA SLE patients. Despite the impact of medications, ancestry made a significant contribution to gene expression profiles. Notably, Differences between AA and EA SLE patients were observed to be similar to those between healthy people of these ancestry groups, and there were fewer differences between males and females of the same ancestry, than between ancestry groups.

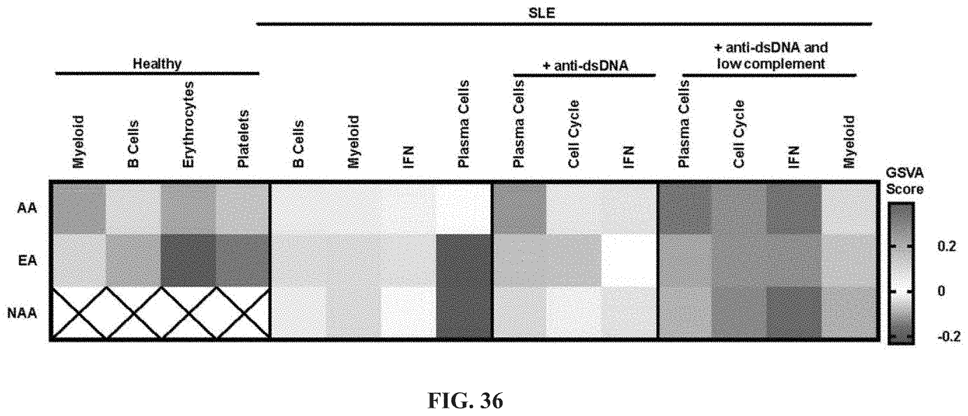

FIG. 36 shows that gene expression is affected by ancestry, SLE autoantibodies, and standard-of-care (SOC) drugs. Average difference in GSVA enrichment scores are shown for healthy subjects. Average GSVA enrichment scores are shown for lupus (SLE) patients. Combinations of different ancestries, specific medications, and autoantibody production are associated with gene expression profiles (FIG. 36). Importantly, ancestry contributes unique features of gene expression, indicating differences in the molecular basis of SLE in these populations. Understanding the contributions of the gene expression signature components may permit a better interpretation of the signatures and their relationship to disease status.

### Example 8: Analysis of Discoid Lupus Erythematosus (DLE) Gene Expression Reveals Dysregulation of Pathogenic Pathways Associated with Infiltrating Immune/Inflammatory Cells

Discoid lupus erythematosus (DLE) is a chronic, scarring inflammatory autoimmune disease of the skin. The precise molecular pathways underlying DLE pathogenesis have not been fully delineated. To obtain a more complete view of the pathologic processes involved in DLE, a comprehensive analysis of gene expression profiles from DLE affected skin was performed.

Microarray gene expression data was obtained from skin biopsy samples of three studies (GSE81071, GSE72535, and GSE52471). Differentially expressed genes (DEGs) between DLE and control were identified by LIMMA analysis. Weighted gene co-expression network analysis (WGCNA) yielded modules of co-expressed genes. Modules correlating to clinical data were prioritized. Correlated modules were interrogated for statistical enrichment of immune and non-immune cell type specific gene signatures. Genes were functionally characterized using a curated immune-specific gene functional category database (BIG-C) and pathways elucidated using IPA®. Queries of a perturbation database (LINCS, Library of Integrated Network-Based Cellular Signatures) were used to identify drugs that could reverse the altered gene expression patterns in DLE.

For each dataset, between 7-12 WGCNA modules had significant correlations to disease. Significant WGCNA module preservation was observed between all three datasets. Non-immune cell types (fibroblasts, keratinocytes, melanocytes) and also Langerhans cells were represented in WGCNA modules negatively correlated with disease. An immune cell signature was observed in WGCNA modules positively correlated to DLE, including DCs, myeloid cells, CD4+ & CD8+ T cells, NK cells, B cells as well as pre- and post-switch plasma cells (PCs). The presence of both Ig-κ and -λ, as well as multiple VL genes suggests the presence of polyclonal PCs. Chemokines that mediate lymphocyte organization and/or recruitment into the skin were identified, including CCL5,7,8 and CXCL9-10,13. Cytokines (TNF, IFNγ, IFNα, IL1β, IL2, IL6, IL12, IL17, IL23, and IL27), signaling molecules (CD40L, PI3K, and mTOR) and transcription factors (NF-κB, NF-AT), as well as cellular proliferation, were evident. IPA® UPR analysis indicated that many of the expressed genes may be secondary to signaling by TNF, IFNγ, IFNα, CD40L, IL1β, IL2, IL6, IL12, IL17, IL23, and IL27. Interestingly, connectivity analysis using LINCS/CLUE identified high-priority drug targets, such as IKZF1/3 (lenalidomide, CC-220), JAK1/2 (ruxolitinib), and HDAC6 (Ricolinostat) may be viable options for therapeutic intervention.

Bioinformatic analysis of DLE gene expression has elucidated many dysregulated signaling pathways potentially involved in the pathogenesis of DLE that may be targeted by novel therapeutic strategies. Further investigation of these signatures may provide an enhanced understanding of the pathogenesis of DLE.

### Example 9: Analysis of Gene Expression from Systemic Lupus Erythematosus (SLE) Synovium Reveals Unique Pathogenic Mechanisms

Arthritis is a common manifestation of systemic lupus erythematosus (SLE), and the efficacy of a new lupus therapy for a given SLE patient often depends on its ability to suppress joint inflammation. Despite this, an understanding of the underlying pathogenic mechanisms driving lupus synovitis remains incomplete. Therefore, gene expression profiles of SLE synovium were interrogated to gain insight into the nature of joint inflammation in lupus arthritis.

Biopsied knee synovia from SLE and osteoarthritis (OA) patients were analyzed for differentially expressed genes (DEGs) and also by Weighted Gene Co-expression Network Analysis (WGCNA) to determine similarities and differences between gene profiles and to identify modules of highly co-expressed genes that correlated with clinical features of lupus arthritis. DEGs and correlated modules were interrogated for statistical enrichment of immune and non-immune cell type-specific signatures and validated by Gene Set Variation Analysis (GSVA). Genes were functionally characterized using BIG-C and canonical pathways and upstream regulators operative in lupus synovitis were predicted by IPA®.

DEGs upregulated in lupus arthritis revealed enrichment of numerous immune and inflammatory cell types dominated by a myeloid phentoype, whereas downregulated genes were characteristic of fibroblasts. WGCNA revealed 7 modules of co-expressed genes significantly correlated to lupus arthritis or disease activity (e.g., as indicated by SLEDAI or anti-dsDNA titer). Functional characterization of both DEGs and WGCNA modules by BIG-C analysis revealed consistent co-expression of immune signaling molecules and immune cell surface markers, pattern recognition receptors (PRRs), antigen presentation, and interferon stimulated genes. Although DEGs were predominantly enriched in myeloid cell transcripts, WGCNA also revealed enrichment of activated T cells, B cells, CD8 T, and NK cells, and plasma cells/plasmablasts, indicating an adaptive immune response in lupus arthritis. Th1, Th2, and Th17 cells were not identified by transcriptomic analysis, although IPA® analysis predicted signaling by the Th1 pathway and numerous innate immune signaling pathways were verified by GSVA. IPA® additionally predicted inflammatory cytokines TNF, CD40L, IFNα, IFNβ, IFNγ, IL27, IL1, IL12, and IL15 as active upstream regulators of the lupus arthritis gene expression profile, in addition to the PRRs IRF7, IRF3, TLR7, TICAM1, IRF4, IRF5, TLR9, TLR4, and TLR3. Analysis of chemokine receptor-ligand pairs, adhesion molecules, germinal center (GC) markers, and T follicular helper (Tfh) cell markers indicated trafficking of immune cell populations into the synovium by chemokine signaling, but not in situ generation of fully-formed GCs. GSVA confirmed activation of both myeloid and lymphoid cell types and inflammatory signaling pathways in lupus arthritis, whereas OA was characterized by tissue repair and damage.

Bioinformatic analysis of lupus arthritis revealed a pattern of immunopathogenesis in which myeloid cell-mediated inflammation dominates, leading to further recruitment of adaptive immune cells that contribute to the ongoing inflammatory synovitis.

### Example 10: Transcriptomic Meta-Analysis of Lupus-Affected Tissues Reveals Shared Immune, Metabolic, and Biochemical Dysregulation

Systemic lupus erythematosus (SLE) affects various organs and tissues, but whether pathologic processes in each organ are distinct or whether dysregulated molecular functions are found in common in all tissues may be unknown. Therefore, a meta-analysis of gene expression profiles in four affected SLE tissues was performed to identify commonly dysregulated pathways.

Gene expression datasets for discoid lupus erythematosus (DLE), lupus arthritis (LA), lupus nephritis (LN) glomerulus (Glom), and LN tubulointerstitium (TI) were obtained from GEO. Differentially expressed genes (DEGs) were identified by LIMMA analysis for each dataset. DEGs from each tissue were analyzed with a multi-pronged bioinformatics approach to elucidate common immune cell infiltrates and common functional categories. These findings were then utilized to form modules of co-expressed genes to determine their enrichment using Gene Set Variation Analysis (GSVA).

All tissues demonstrated the presence of immune cells with the fewest immune cell transcripts in LN TI. Analysis of bulk gene expression revealed enrichment of antigen presenting cells (APCs), monocytes, and myeloid cells in all four tissues. Notably, enrichment of B cells, plasma cells, germinal center (GC) B cells, and CD8 T cells was only detected in DLE and LA. All four tissues demonstrated upregulated immune activity, including interferon-stimulated genes, pattern recognition receptors (PRRs), and antigen presentation (MHC Class II). Pro-apoptosis genes were also found enriched in DLE, LN Glom, and LN TI. A generalized decrease in biochemical processes was found in all four tissues, and a specific decrease in both fatty acid biosynthesis and the tricarboxylic acid cycle was found in DLE and LN. Ingenuity Pathway Analysis (IPA®) further confirmed the activation of Dendritic Cell Maturation, Interferon, NFAT Regulation of Immune Response, PRRs, and TH1 signaling pathways in all four tissues. Additionally, IPA demonstrated cholesterol biosynthesis was decreased in all tissues except LA.

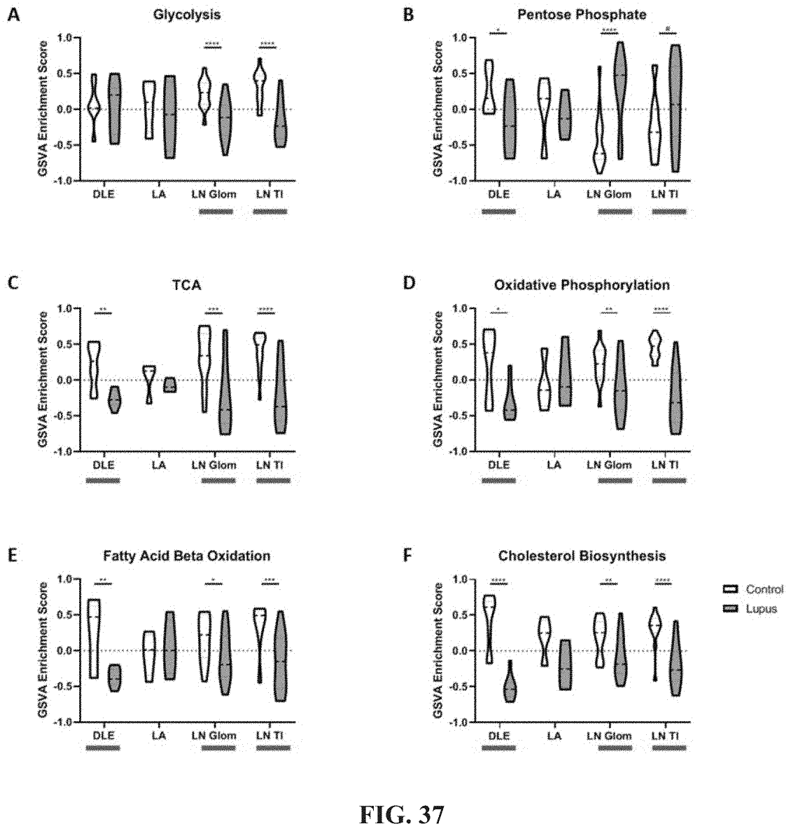

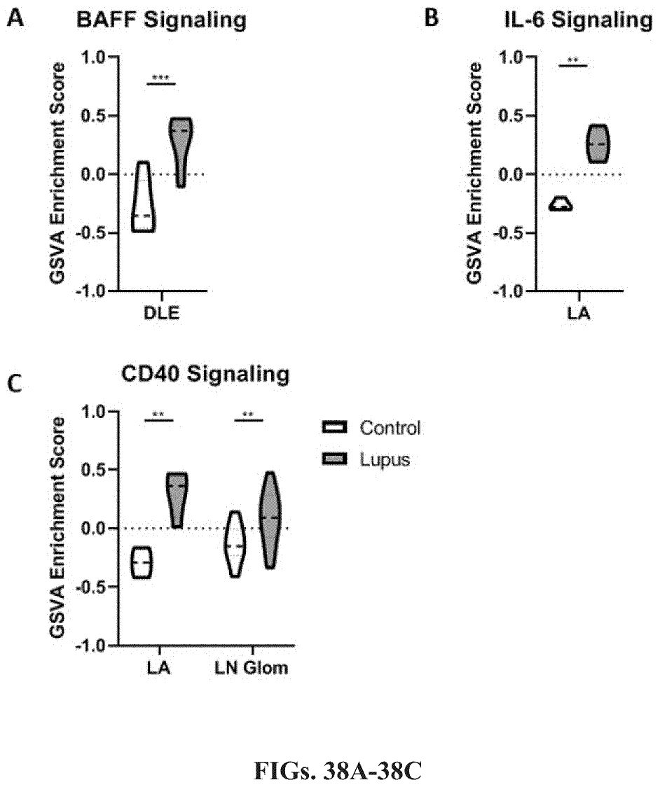

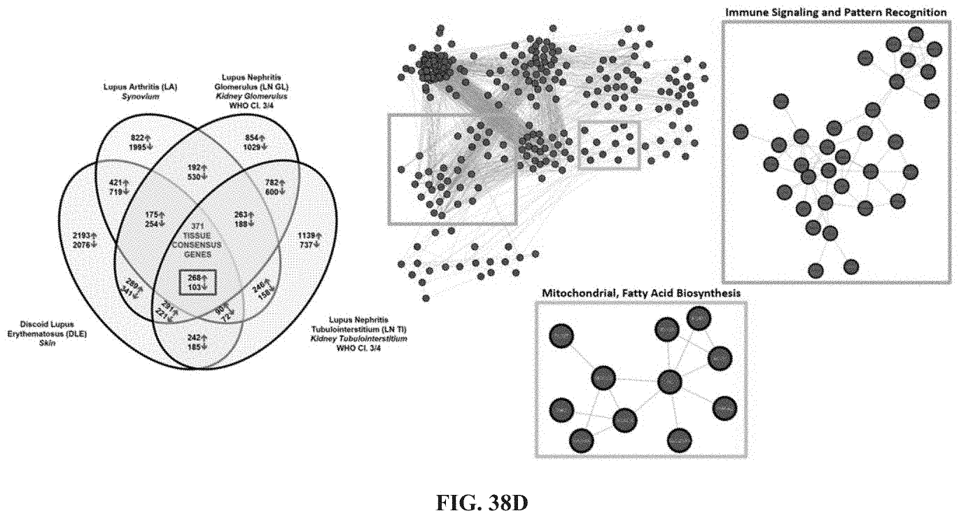

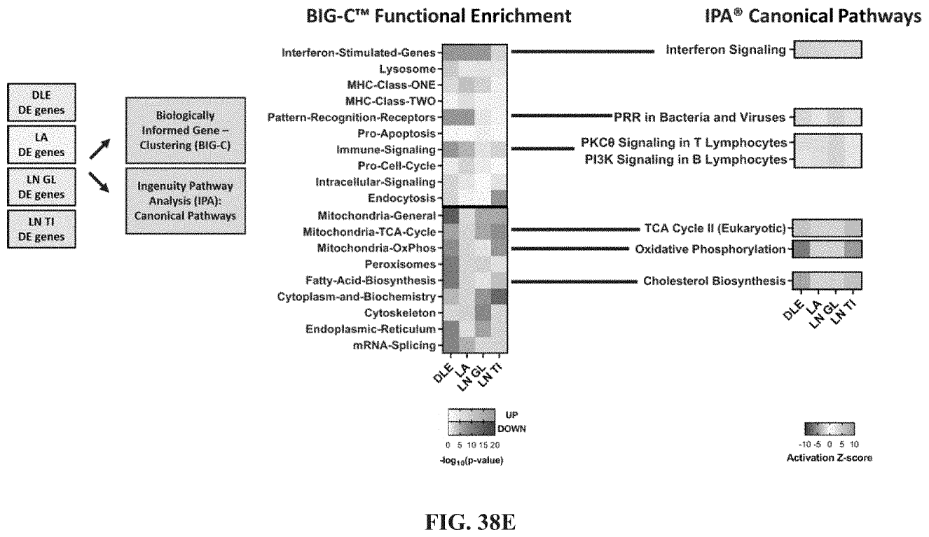

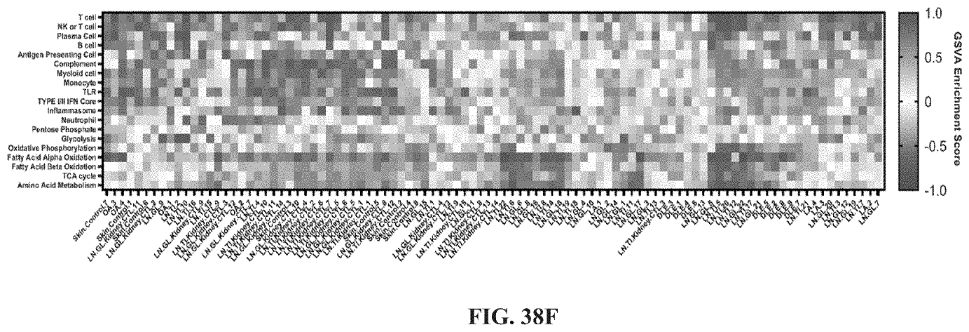

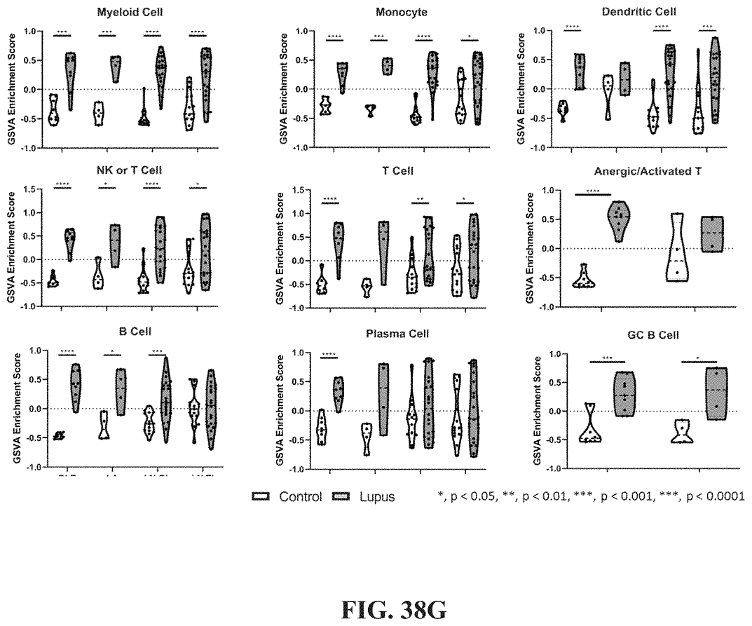

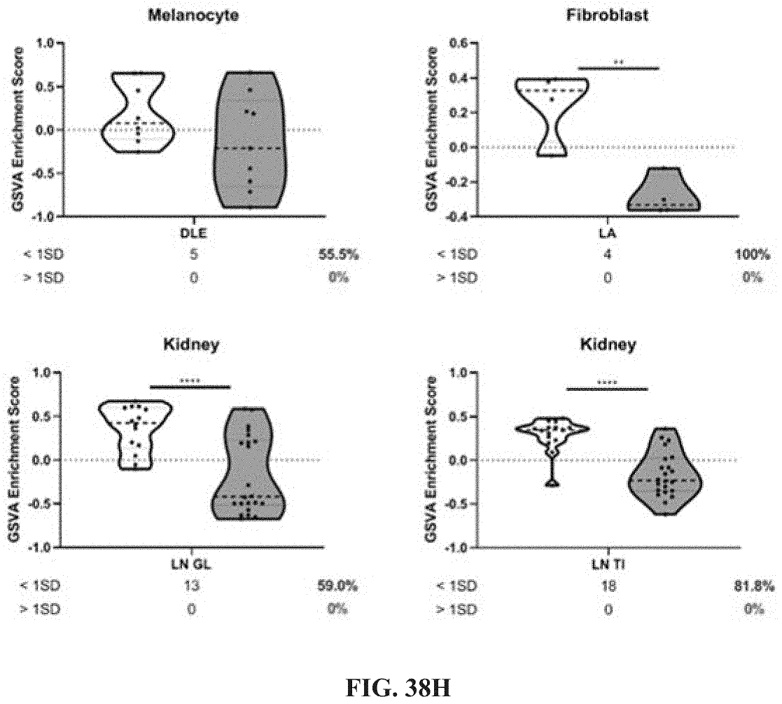

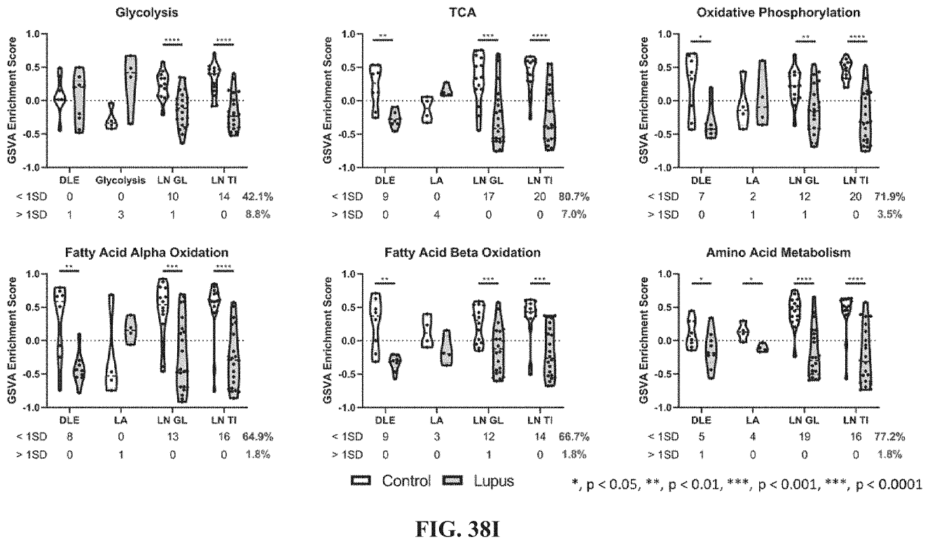

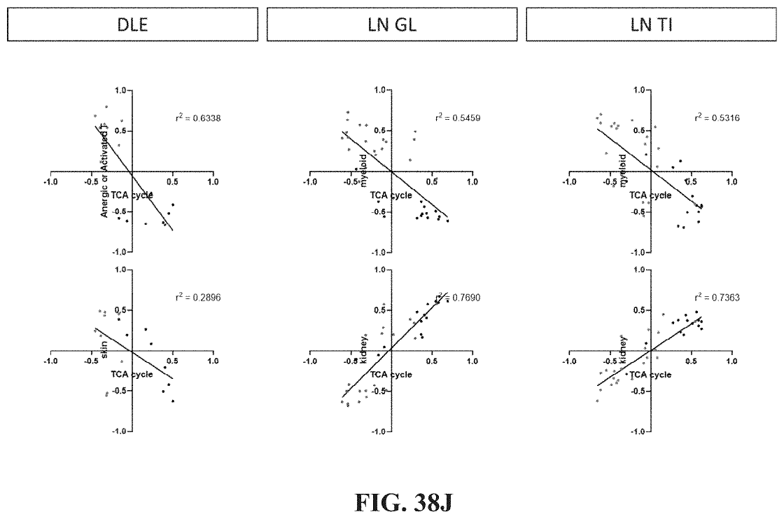

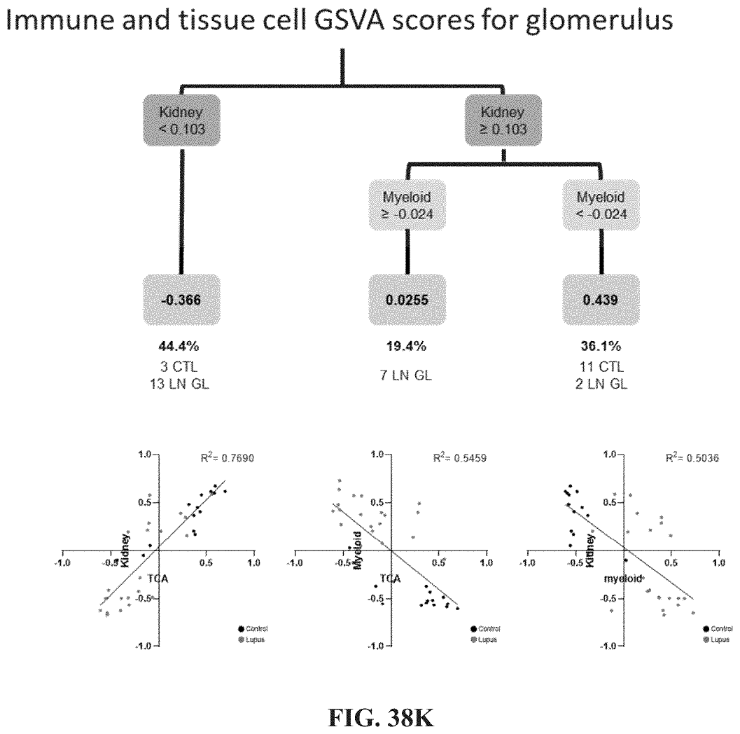

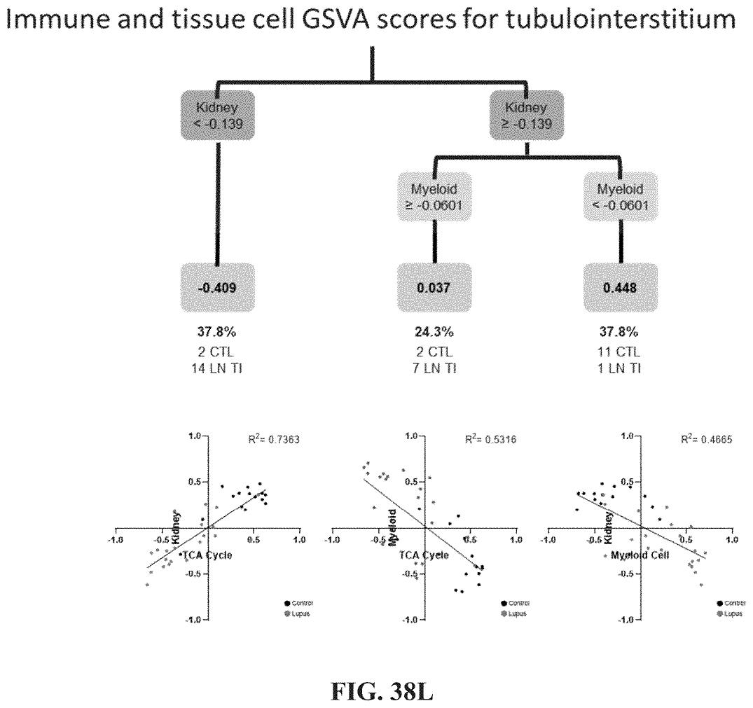

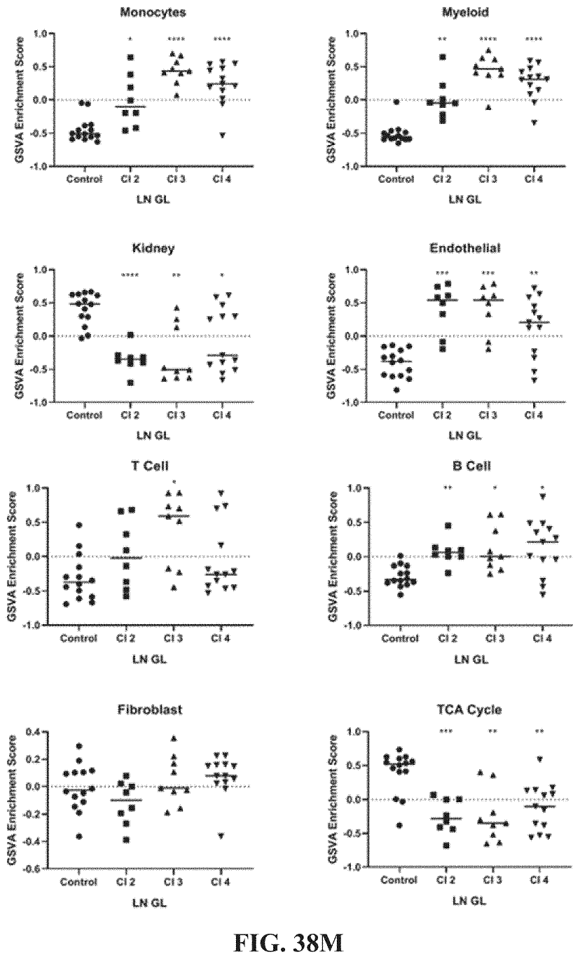

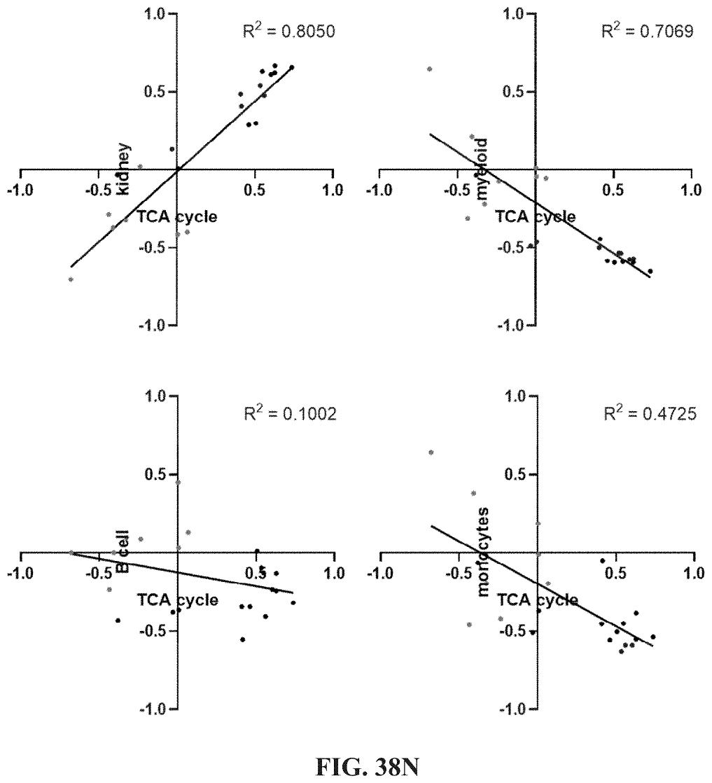

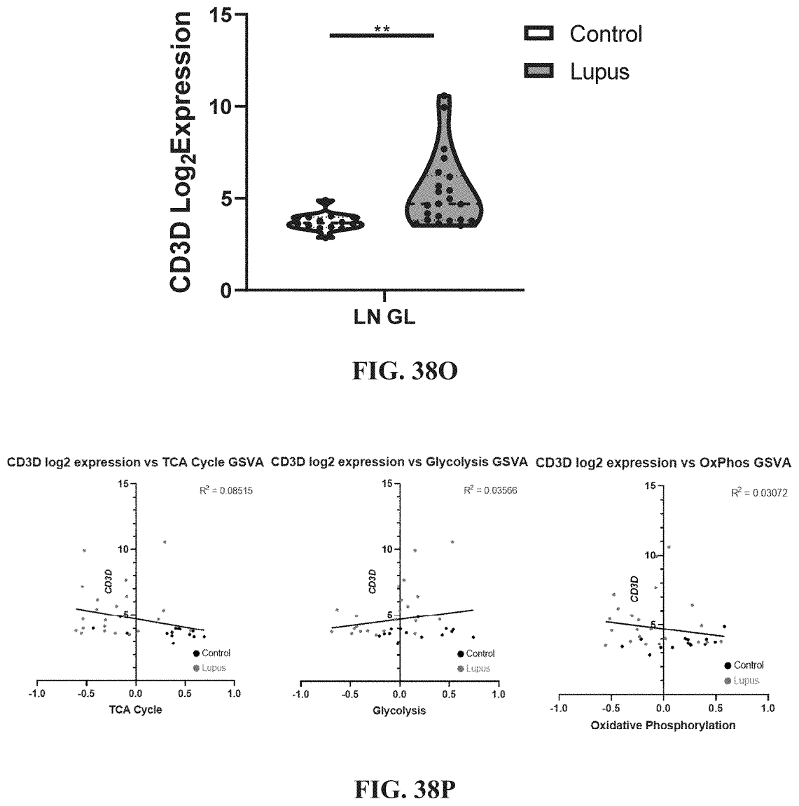

To confirm the aforementioned cellular infiltrates and aberrant pathways, as well as additional pathways, were operative in individual SLE tissues, GSVA was used to analyze enrichment of gene modules in patient samples. As shown in Table 18 and FIGS. 37-38, specific abnormalities were found in the majority of tissues, including enrichment of myeloid cells/monocytes, APCs, and GC B cells, whereas others were observed in some but not all tissues.

TABLE 18
Percentages of SLE tissue samples with GSVA
enrichment of specific immune cell modules

| | DLE | LA | LN Glom | LN TI |
|---|---|---|---|---|
| Antigen Presenting Cell | 66.67% | 75.00% | 77.27% | 63.64% |
| Monocyte | 88.89% | 100.00% | 95.45% | 59.09% |
| Myeloid Cell | 77.78% | 100.00% | 81.82% | 68.18% |
| Germinal Center B Cell | 77.78% | 100.00% | 54.55% | 77.27% |
| Plasma Cell | 88.89% | 75.00% | 50.00% | 45.45% |

FIG. 37 contains plots showing that GSVA demonstrates metabolic dysregulation in individual SLE affected tissues. GSVA enrichment scores were calculated for (A) glycolysis, (B) pentose phosphate, (C) tricarboxylic acid cycle (TCA), (D) oxidative phosphorylation, (E) fatty acid beta oxidation, and (F) cholesterol biosynthesis modules in DLE, LA, LN Glom, and LN TI. Significant enrichment of tissue control to SLE affected tissue or SLE affected tissue to tissue control was determined using the Welch's t-test. The red bar represents enrichment of SLE tissue over control, and the blue bar represents enrichment of tissue control over SLE tissue. # p<0.1 *p<0.05, ** p<0.01, *** p<0.001, ****<0.0001.

FIGS. 38A-38C contains plots showing that GSVA reveals potential pathways for therapeutic targeting in lupus affected tissues. Measures are shown for drug pathways significantly enriched in SLE affected tissue compared to control tissue as determined using the Welch's t-test for B cell activating factor (BAFF) (FIG. 38A), interleukin (IL-6) (FIG. 38B), and CD40 signaling in DLE, LA, and LN Glom (FIG. 38C). ** p<0.01, *** p<0.001.

FIG. 38D shows that genes commonly dysregulated in lupus tissues identified immune processes and cellular metabolism.

FIG. 38E shows that functional grouping and pathway analysis of DE genes expressed in lupus tissues revealed immune and metabolic abnormalities in common.

FIG. 38F shows that similar cellular and metabolic signatures were observed in lupus tissues.

FIG. 38G shows that increased immune/inflammatory cell signatures were observed in lupus tissues.

FIG. 38H shows that decreased tissue stromal cell signatures were observed in lupus tissues.

FIG. 38I shows that decreased metabolic signatures were observed in lupus tissues.

FIG. 38J contains plots showing the correlation between immune/inflammatory or tissue cell signature and metabolic signature in DLE and LN (LN GL and LN TI).

FIG. 38K-38L shows that Classification and Regression Trees (CART) analysis predicted the contributors to metabolic dysfunction.

FIG. 38M shows that Class 2 LN glomerulus demonstrated similar metabolic defects, indicating dysregulation is linked to stromal cells.

FIG. 38N contains plots showing the correlation between tissue or immune/inflammatory cell signature and metabolic signature for Class 2 LN glomerulus.

FIG. 38O-38P contain plots showing that metabolic changes were not correlated with T Cells in LN GL.

Common cellular infiltrates and molecular pathways were found in all affected tissues, suggesting commonalities in lupus organ pathogenesis. However, certain cell types and signaling were predominant in some tissues over others and GSVA illustrated heterogeneity between patients. Together this analysis informs a tissue-specific model of lupus immunopathogenesis and metabolic dysfunction with common and unique features and highlights the importance of patient specific identification of dysfunctional pathways in lupus organ pathogenesis.

### Example 11: Analysis of Lupus Nephritis (LN) Gene Expression Reveals Dysregulation of Pathogenic Pathways Activated within Infiltrating Cells

Lupus nephritis (LN) is a serious complication of SLE that affects about 20-40% of all lupus patients and leads to kidney damage, end-stage renal disease, and patient mortality. Despite advances in therapy, progression to end stage renal disease may not be affected. Therefore, it is important to re-consider the pathogenic mechanisms involved in LN as a basis for development of more effective therapies. A multi-pronged approach was performed to characterize LN via bioinformatic analysis of gene expression data obtained from kidney biopsies.

Genomic expression profiling data of LN patient biopsies, microdissected into glomerulus and tubulonterstitium (TI), was sourced from GSE32591 via the GEO database. Differentially expressed genes (DEGs) detected in LN-derived samples relative to samples from healthy individuals were interrogated for cell infiltrate composition using gene set variation analysis (GSVA) against a curated database of immune and non-immune cell type signatures (I-SCOPE, T-SCOPE). Weighted gene co-expression network analysis (WGCNA) was performed to generate gene modules correlated to clinical variables. DEGs were further functionally characterized using a curated immunity-specific gene functional category database (BIG-C) and IPA signaling pathway analysis software. Queries of the perturbation database (LINCS, Library of Integrated Network-Based Cellular Signatures) were used to identify possible upstream regulators of altered gene expression patterns in LN samples as well as to identify drugs that could reverse abnormal gene expression profiles.

WGCNA produced 6 gene modules (3 glomerulus, 3 TI) positively correlated with disease stage, as measured by WHO class. These modules were enriched in signatures for several immune cell types, including granulocytes, pDC, DC, myeloid cells, CD4+/CD8+ T cells, and B cells. Additionally, the presence of both IG-κ and -λ, as well as VL genes and detection of pre- and post-switch PCs as indicated by IgM, IgD, and IgG1 Ig Heavy Chain genes indicate polyclonal PC infiltration. Podocyte signatures were detected as enriched in WGCNA modules negatively correlated with WHO class. Chemokines and pathways that mediate lymphocyte proliferation, organization, and/or recruitment into lupus kidney tissue were detected as enriched via BIG-C and IPA analysis, including the cytokines TNF, IL1β, IL2, IL6, IL12, IL17, IL23, and IL27 and signaling pathways including CD40L, PI3K, NF-κB, NF-AT, and p70S6K. IPA upstream regulator analysis indicated ongoing signaling by cytokines such as TNF, IFNγ, IFNα, CD40L, IL1β, IL2, IL6, and IL17. Interestingly, connectivity analysis using LINCS elucidated high-priority drug targets such as IFNβ (PF-06823859), IL12 (Ustekinumab), and S1PR (Fingolimod) that may be suitable options for therapeutic intervention.

Bioinformatic analysis of LN gene expression highlighted several dysregulated signaling pathways that can form the targets of novel therapeutic strategies, and further elucidation of these signatures may enhance clinical surveillance and diagnosis of LN to improve patient outcomes.

### Example 12: Integration of Genetic Data, Molecular Pathway Analysis, and Differential Expression to Delineate the Impact of Ancestral Differences on Lupus

Systemic lupus erythematosus (SLE) is a multi-organ autoimmune disorder with a prominent genetic component. In many cases, individuals of African-Ancestry (AA) experience the disease more severely and with an increased co-morbidity burden compared to European-Ancestry (EA) populations. However, the relationship between genetics, molecular pathways, and disease severity may not have been fully delineated. AA and EA SLE-associated single nucleotide polymorphisms (SNPs) were examined and linked via expression quantitative trait loci (eQTL) across multiple tissues to genes with altered expression (E-Genes). Putative EA and AA E-Gene signatures were coupled with SLE differential expression (DE) datasets and upstream regulators to map candidate molecular pathways. Together, these genetic and gene expression analyses enable a better understanding of how the identified SNPs may contribute to aberrant immune function as well as the influence of ancestry on the genetic basis of SLE.

SLE Immunochip studies may be performed to identify SNPs significantly associated with SLE in AA (2,970 cases; 2,452 controls) and EA (6,748 cases; 11,516 controls) cohorts. eQTL mapping identified E-Genes from SLE SNPs and their ancestry-specific SNP proxies (based on linkage disequilibrium) via the GTEx database. For both ancestral groups, E-Gene lists were examined for the significant enrichment of gene ontogeny (GO) terms, canonical IPA® (Qiagen) pathways and BIG-C™ categories. Next, the gene expression profiles of predicted E-Genes were analyzed across multiple SLE DE datasets, including those from blood and multiple tissues. Differential expressed genes (DEGs) were identified and subjected to pathway analysis with IPA®, clustering using MCODE, and visualization in Cytoscape with the ClusterMaker2 plugin. Drug candidates targeting E-Genes, DEGs and upstream regulators (UPRs) were identified using CLUE, IPA®, and STITCH.

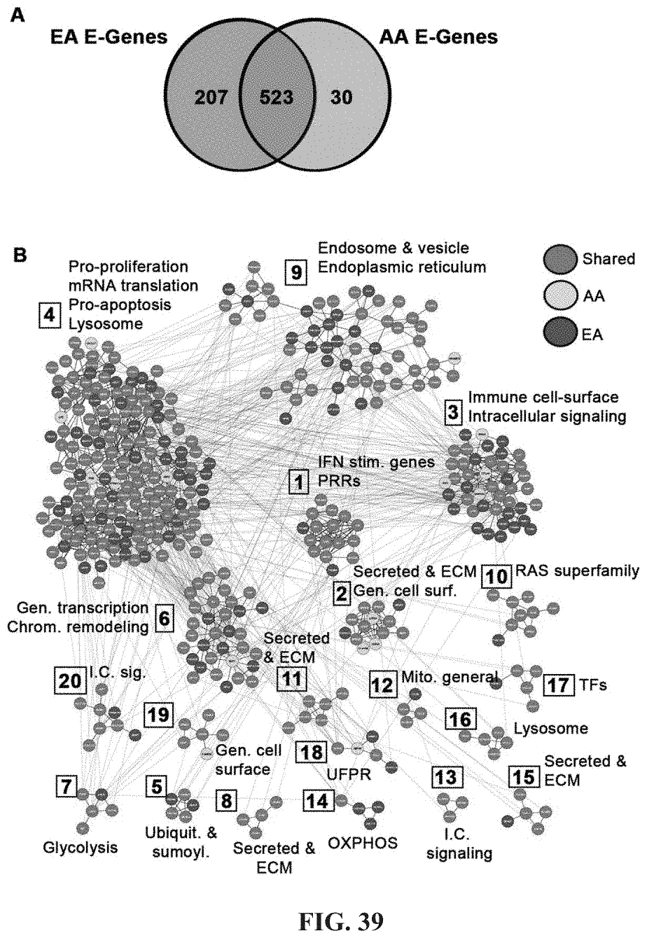

As shown in FIG. 39, a total of 908 Immunochip SNPs were mapped to 252 eQTLs and coupled to 760 E-Genes (207 in EAs, 30 in AAs, 523 shared). The figure shows (A) a Venn of E-Gene overlap and (B) a Cytoscape visualization of E-Gene PPI networks using MCODE clustering. Significant BIG-C functional categories for individual modules are listed. Shared E-Genes were highly enriched in interferon signaling, whereas EA E-Genes were associated with nucleotide degradation and AA E-Genes were linked to multiple biosynthesis and intracellular signaling pathways (e.g., retinol biosynthesis and AMPK signaling). Protein-protein interaction (PPI) networks of clustered EA, AA, and shared E-Genes illustrate the high degree of ancestral overlap evident within each E-Gene set. Clustering analysis of all DE E-Genes and IPA-predicted UPRs highlight disease-associated pathways that are both shared and ancestry-specific. Drug candidate comparison identified a total of 115 drugs targeting EA, AA, and shared E-Genes and their molecular pathways.

Using a bioinformatics-based approach that utilizes pathway analysis and gene expression data, ancestry-dependent and ancestry-agnostic candidate causal targets in SLE were discovered. These SLE targets may be suitable for further investigation and analysis using drug discovery tools to identify therapies with potential to impact disease processes within and across specific populations.

### Example 13: Type I and Type II IGS are Present in SLE Cells and Tissues

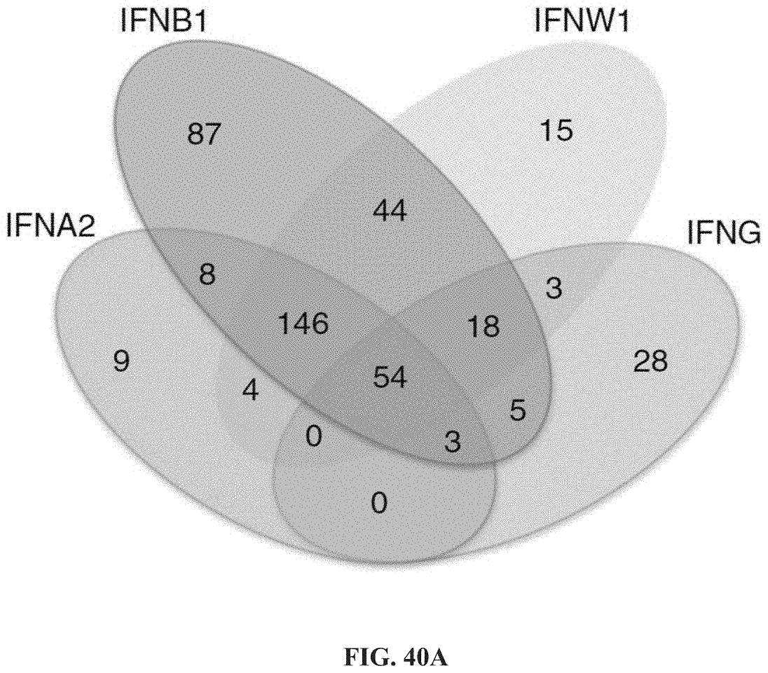

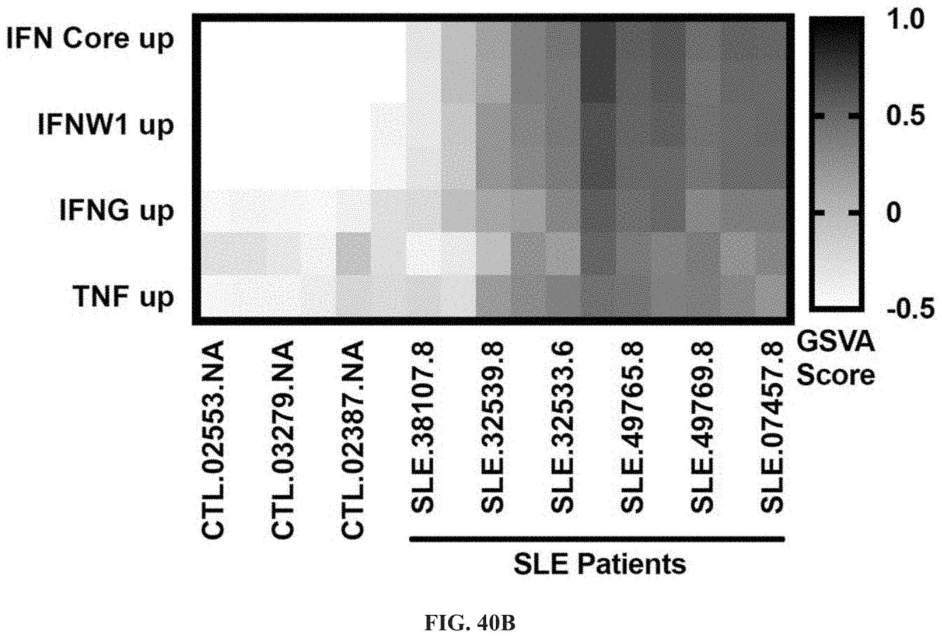

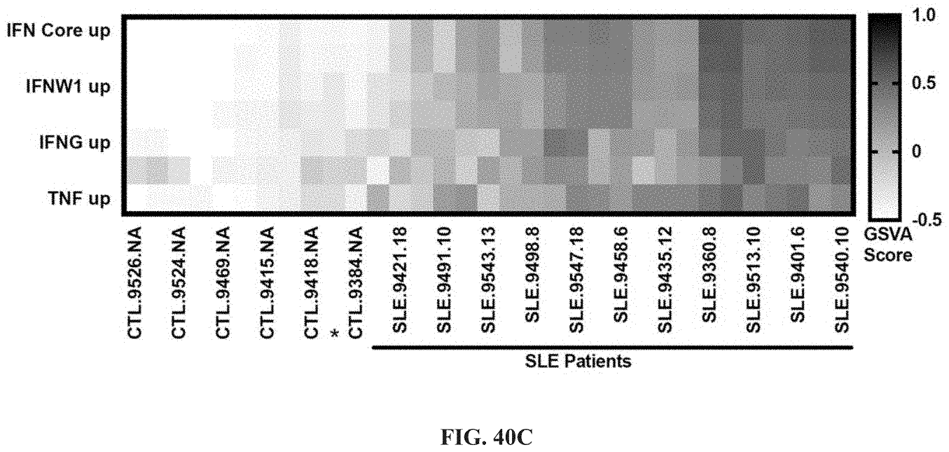

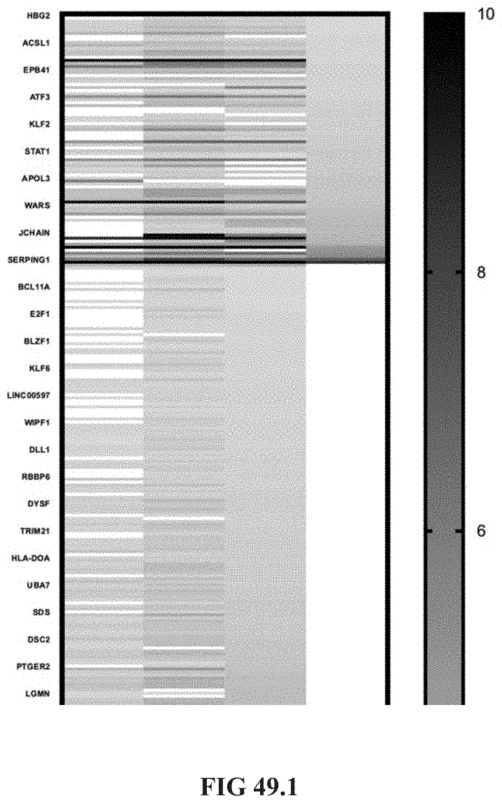

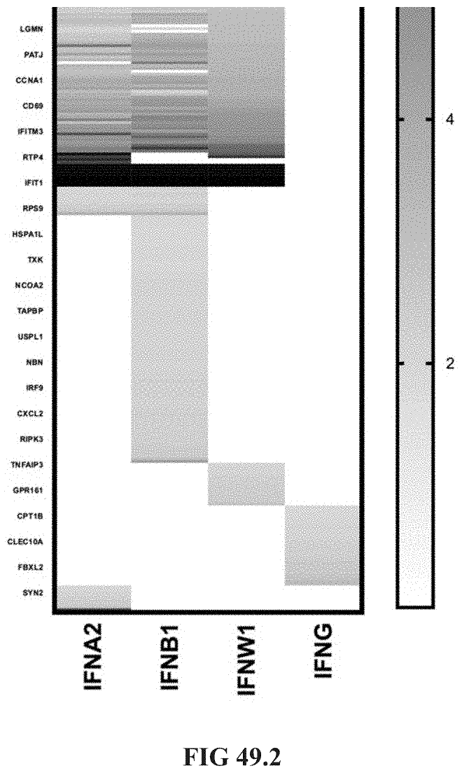

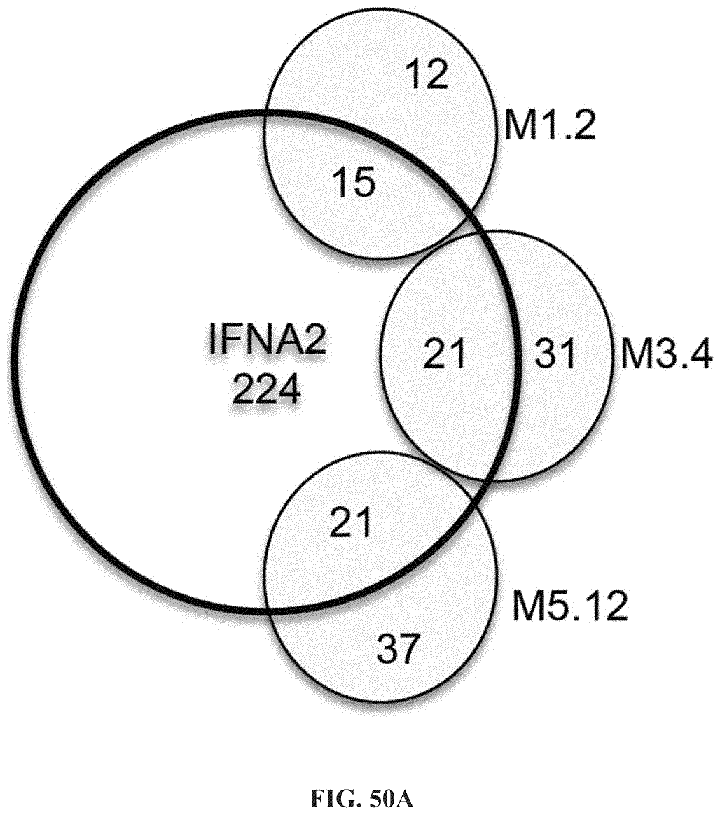

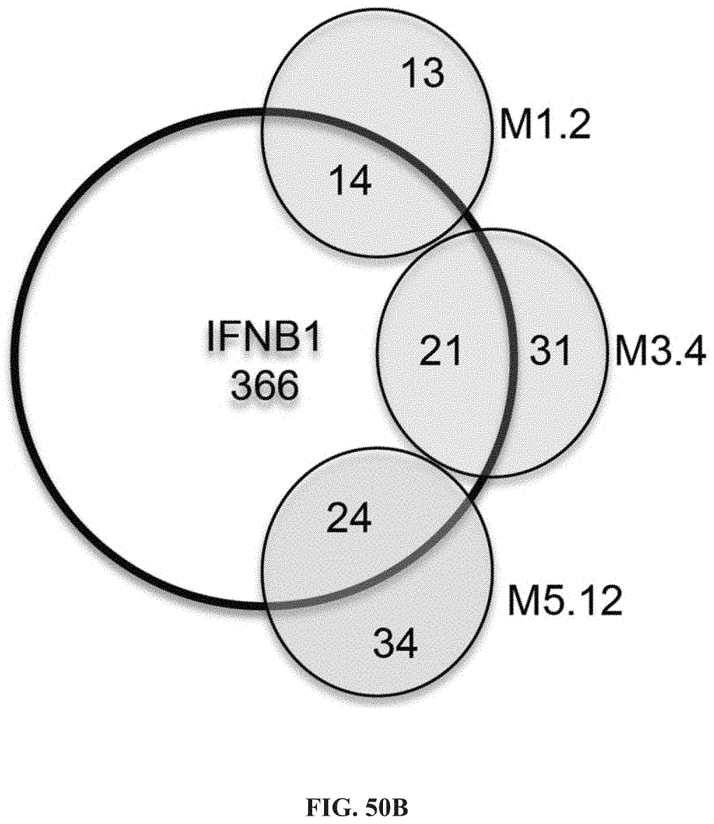

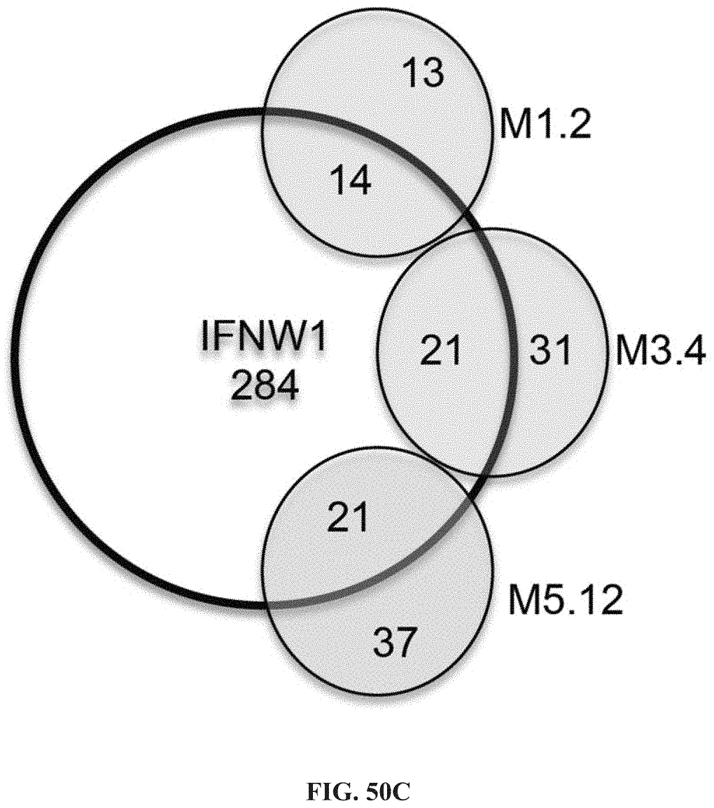

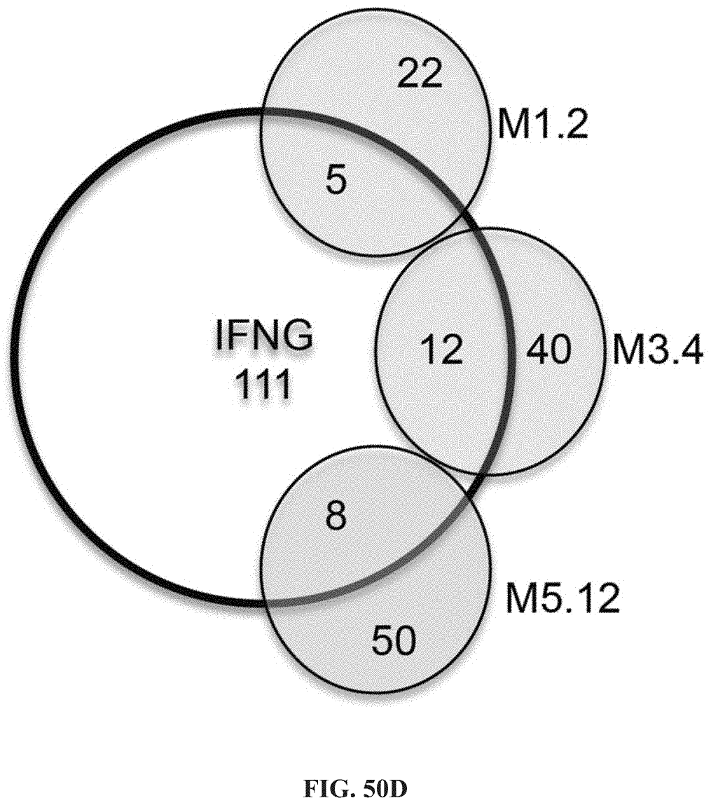

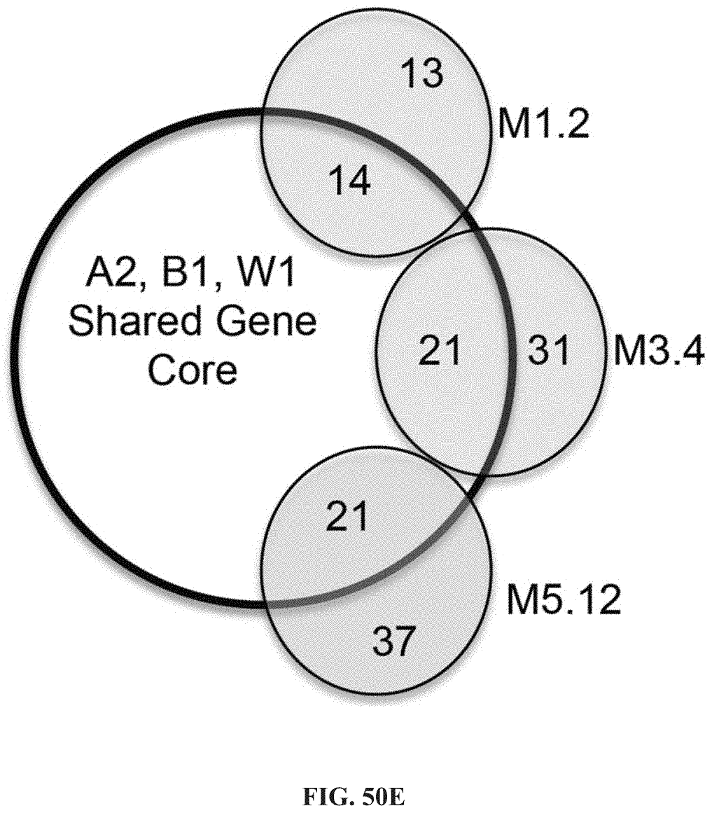

A bioinformatic approach was used to define the subtype of interferon (IFN) in systemic lupus erythematosus (SLE) patients using microarray data derived from publicly available datasets and collaborators. Reference datasets of the IGS were obtained (e.g., as described by Waddell et al.), and included genes induced by the in vitro stimulation of normal human peripheral blood mononuclear cells (PBMC) with IFNA2, IFNB1, IFNW1, or IFNG, and as controls the signatures induced by TNF (tumor necrosis factor) or IL12 (interleukin-12). FIG. 40A depicts a 54 transcript shared type I and type II IFN gene signature (IGS) and a 200 transcript shared type I IGS (IFN Core; Tables 20-30). Each IFN also induced a unique IGS and suggested an approach to determine the predominant type of IFN in SLE patient affected tissues (FIG. 49). Of note, comparison of the IFN induced PBMC transcripts to the three IFN modules previously described by Chiche-Chaussabel demonstrated that the transcripts in common were in the shared IFN core signature and thus the different Chiche-Chaussabel IFN modules did not appear to represent modules induced by specific IFNs (FIGS. 50A-50E). Chiche-Chaussable modules are described by, for example, Chiche, L. et al. Modular transcriptional repertoire analyses of adults with systemic lupus erythematosus reveal distinct type I and type II interferon signatures. Arthritis Rheumatol. 66(6):1583-95(2014), which is hereby incorporated by reference in its entirety.

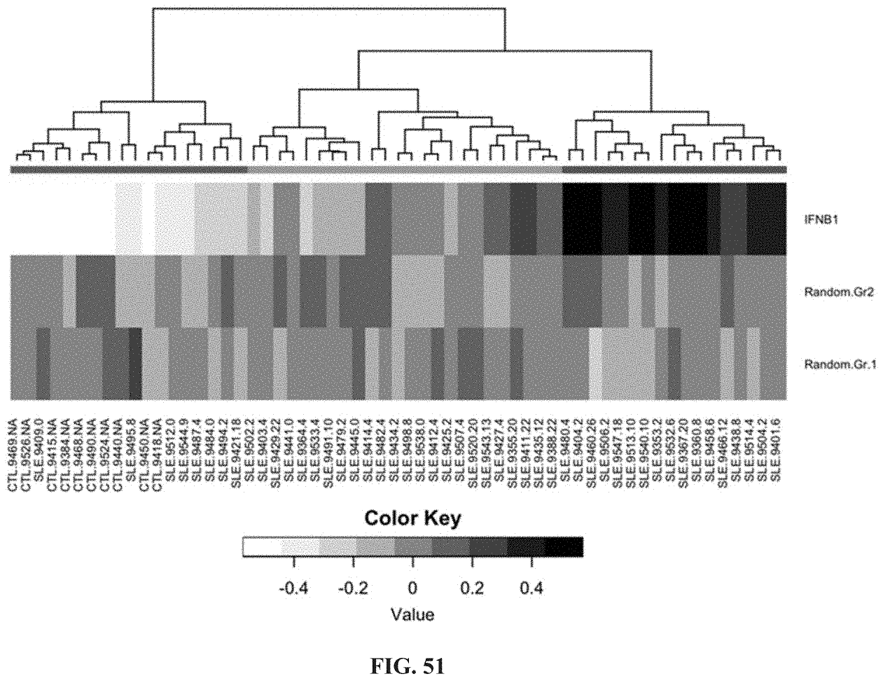

Gene Set Variation Analysis (GSVA) using the induced transcripts for IFNA2, IFNB1, IFW1, IFNG, TNF, IL12, and the IFN Core signature (Tables 20-27 and 30) was employed to determine the relative enrichment of these signatures in SLE patient and control WB or PBMC. GSVA is an unsupervised methodology that calculates enrichment scores between −1 and 1 for groups of genes potentially co-expressed in individual subjects. Because GSVA normalizes the log 2 expression data and allows incorporation of healthy control values in the calculation to standardize the enrichment scores, GSVA may mitigate against strong batch effects in microarray data and may allow a direct comparison of enrichment scores across multiple datasets. Heatmap visualization of the calculated GSVA enrichment scores demonstrated that patients had highly enriched signatures for IFNA2, IFNB1, IFNW1, IFNG, and the IFN core signature, and that most SLE patients were separated from healthy controls (HC) by these signatures. In most SLE patients, the GSVA enrichment scores were the strongest for the type 1 IFNs compared to IFNG, TNF, or IL12. However, some patients had no type I or type II IGS, but did possess a TNF or IL12 signature (FIG. 1b, c). Enrichment of random groups of genes did not separate SLE patients from controls (Tables 28-29; FIG. 51).

### Example 14: Prominent Enrichment of the IFNB1 Signature in SLE Skin and Synovium

As shown in Example 13, both type I and II IFN signatures were enriched in SLE WB and PBMC. Next, Gene Set Variation Analysis (GSVA) was employed to determine whether these signatures were also enriched in SLE affected tissues.

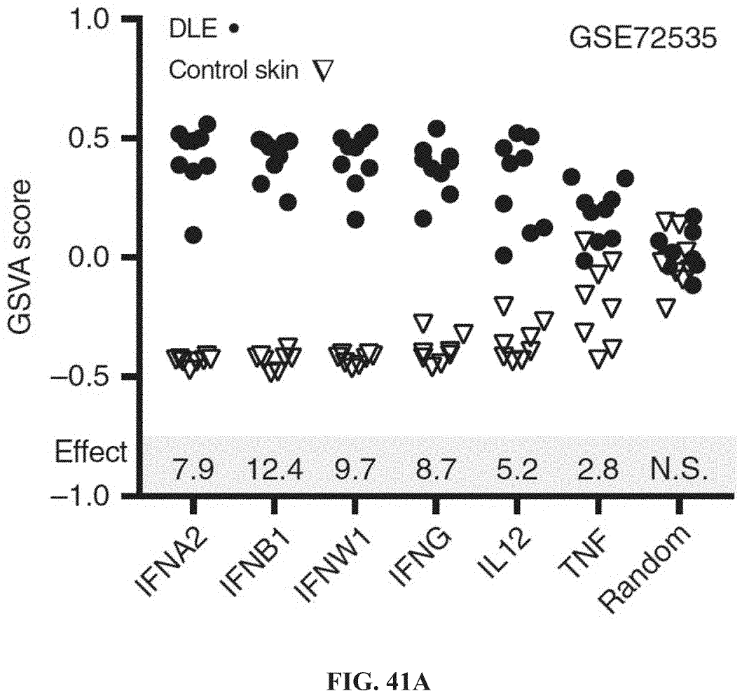

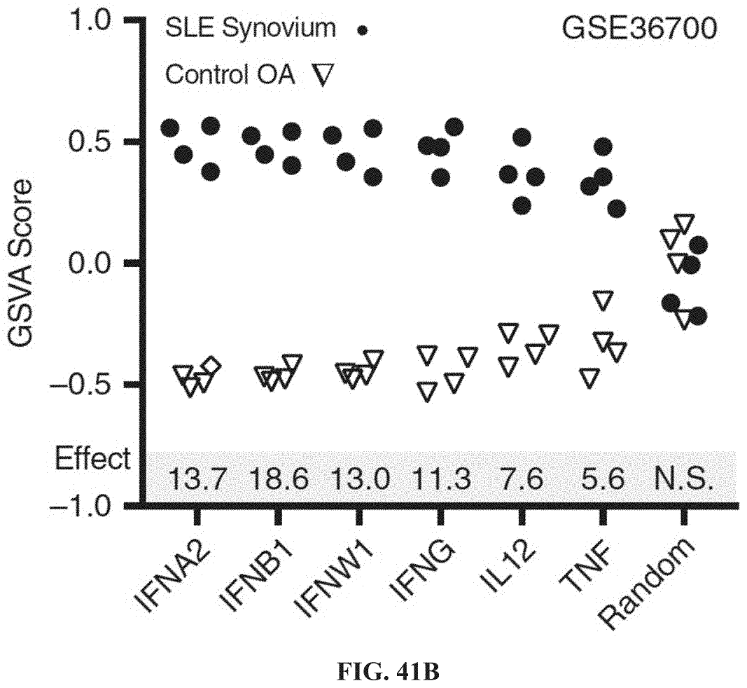

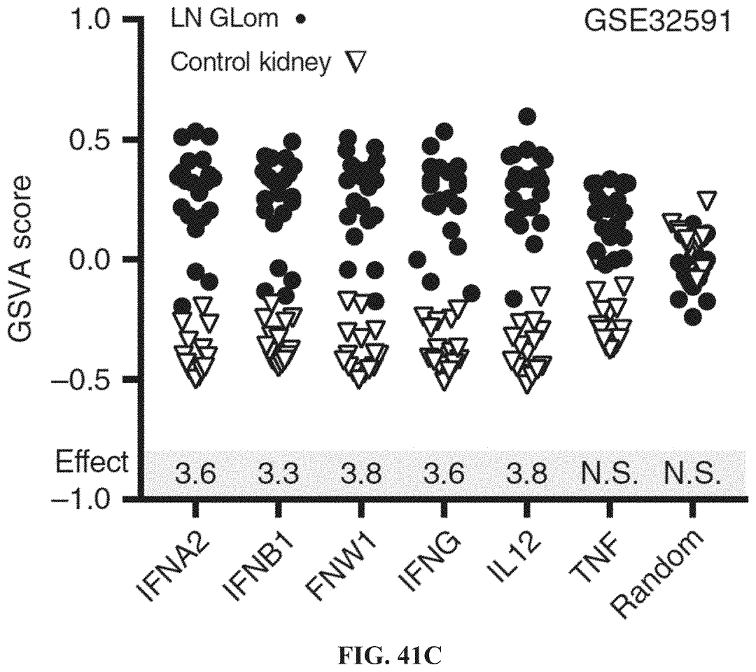

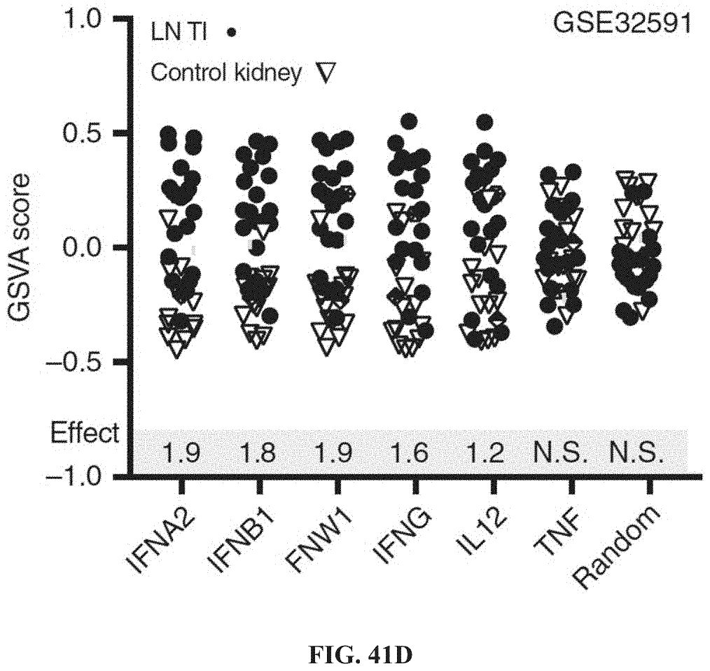

GSVA enrichment scores were calculated using the IFN signatures, and they also separated SLE affected organs from healthy controls (HC). Discoid lupus erythematosus (DLE) was significantly separated from control skin by all of the signatures (p<0.05); IFNB I had the highest effect size (Hedge's g=12.4) followed by IFNW1 (g=9.7), IFNG (g=8.7), IFNA2 (g=7.9), IL12 (g=5.2) and TNF (g=2.8) (FIG. 41A). In SLE synovium, all six signatures were also significantly enriched in SLE patients compared to control osteoarthritis (OA) tissue (p<0.05). In particular, the effect size was the greatest for the IFNB1 signature (g=18.6), followed by IFNA2 (g=13.7), IFNW1 (g=13), IFNG (g=11.3), IL12 (g=7.6), and TNF (g=5.6) (FIG. 41B).

In kidney tissue from SLE patients with Class III and IV lupus nephritis (LN) glomerulus (Glom) (FIG. 41C) and tubulointerstitium (TI) (FIG. 41D), there was no significant TNF enrichment but the other five signatures were significantly enriched in SLE patients compared to controls (p<0.05). The effect size calculations were more than 50% less than those calculated for the DLE and SLE synovium, and five SLE patients had no IGS. IFNW1 had the highest effect size values for LN (Glom g=3.8, TI g=1.9), followed by IL12 (Glom g=3.8, TI g=1.2), IFNG (Glom g=3.6, TI g=1.6), IFNA2 (Glom g=3.6, TI g=1.9), and IFNB1 (Glom g=3.3, TI g=1.8).

Reference datasets for (i) the IFNB1 signature from DE analysis of WB from multiple sclerosis (MS) patients chronically treated with recombinant IFNB1 compared to untreated MS patients (MS-IFNB1) and (ii) the IFNA2 signature derived from DE analysis of PBMC from hepatitis C (HepC) patients six hours after treatment with IFNA2 compared to PBMC from the same HepC patients before treatment (HepC-IFNA2) were used to confirm the relative IGS found in SLE affected tissues. The overlap of the MS-IFNB1 and HepC-IFNA2 signatures with the PBMC-derived IFNA2, IFNB1, and IFNW1 signatures demonstrated large numbers of unique transcripts for the MS-IFNB1 (111) and HepC-IFNA2 (157) signatures and raised the question of whether the transcripts were indeed directly induced by IFN. The Interferome was used to determine whether the transcripts were IFN inducible, and 87.5% of the induced MS-IFNB1 and 56% of the induced HepC-IFNA2 transcripts were identified as type I IFN genes.

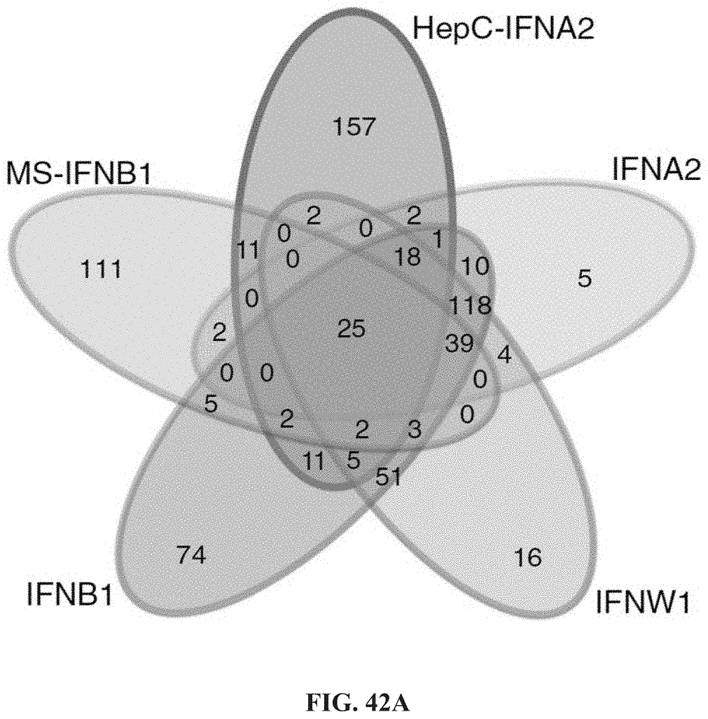

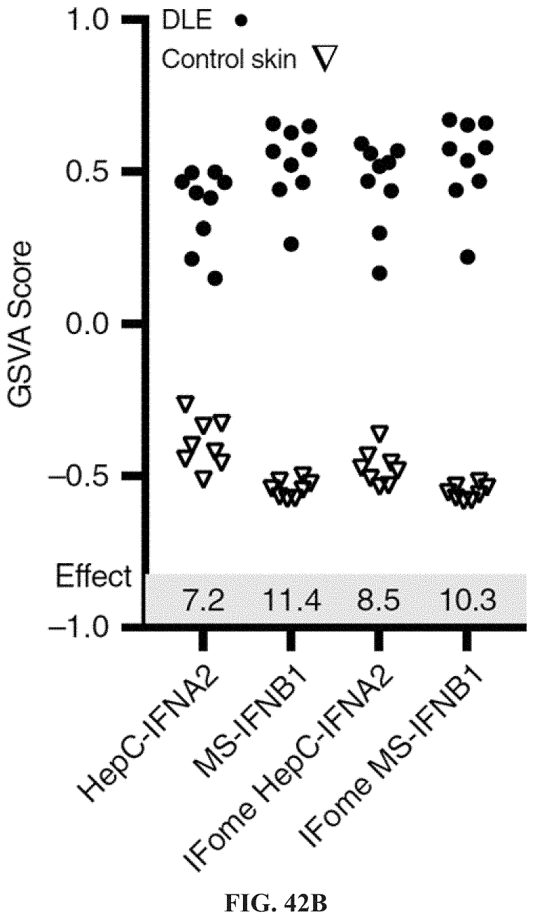

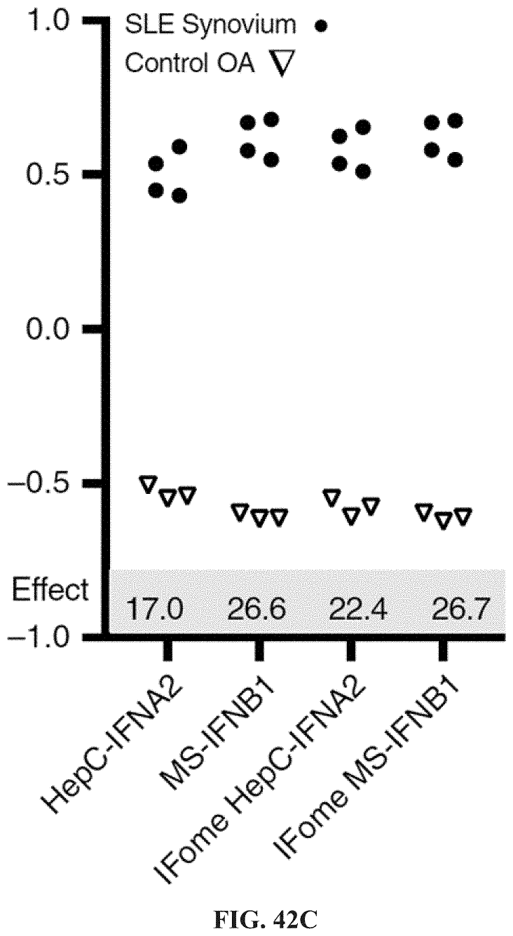

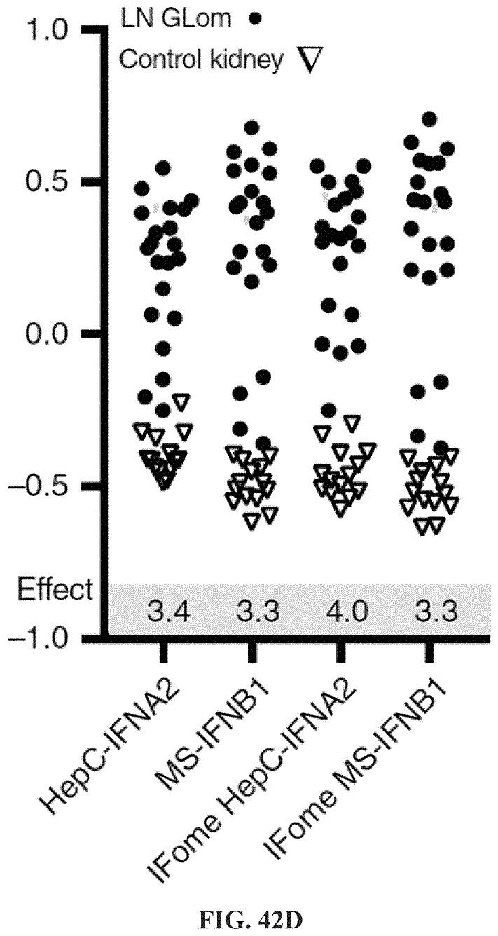

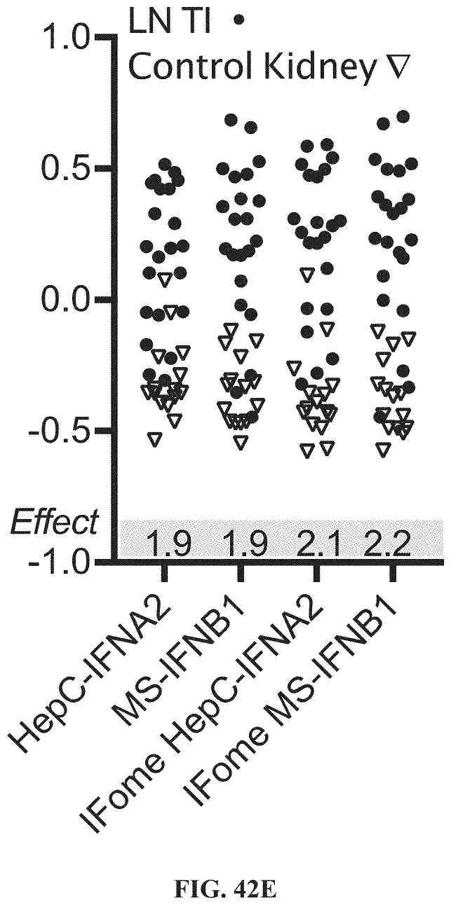

GSVA was carried out on the four SLE-affected tissues using four signatures: MS-IFNB1, HepC-IFNA2, and each of these signatures with only the Interferome database confirmed transcripts (IFome). GSVA revealed that all signatures were significantly enriched in all four SLE affected tissues compared to control tissues (p<0.05). Similar to the pattern seen with the PBMC-derived signatures, the MS-IFNB1 signature had higher effect size values for both DLE (g=11.4) and synovium (g=26.6) compared to the HepC-IFNA2 values for DLE (g=7.2) and synovium (g=17). Removal of the transcripts not listed in the Interferome did not change the effect size values for the MS-IFNB1 signature but increased the HepC-IFNA2 signature for both DLE and synovium (FIGS. 42B-42C). Similar to the results for the PBMC-derived IFN signatures, LN Glom and TI, five patient tissues had no IGS and the calculated effect sizes for HepC-IFNA2 (Glom g=3.4, TI g=1.9) and MS-IFNB1 (Glom g=3.3, TI g=1.9) were lower than for DLE and synovium (FIGS. 42D-42E).

The comparative ability of the different IFN signature enrichment scores to discriminate SLE from control tissue was determined by two-way ANOVA. IFNA2, -IFNB1, -IFNW1, -IFNG, MS-IFNB1 and HepC-IFNA2 downstream signatures were all significantly discriminatory (p<0.05) between SLE and controls compared to random signatures for all four tissues. Notably, the MS-IFNB1 signature in the DLE and Synovium significantly discriminated (p<0.05) between SLE and control compared to all other IFN signatures except for IFNA2 in DLE.

An orthogonal approach was taken by calculating Z scores using both increased and decreased transcripts from PBMC-derived IFN or the MS-IFNB1 signatures to determine the most likely IFN active in SLE patient WB, PBMC, and affected tissues. As controls for Z score calculations, a sepsis microarray dataset and a dermatomyositis microarray dataset were included in this analysis because these conditions have well described roles for either TNF or IFNB1.

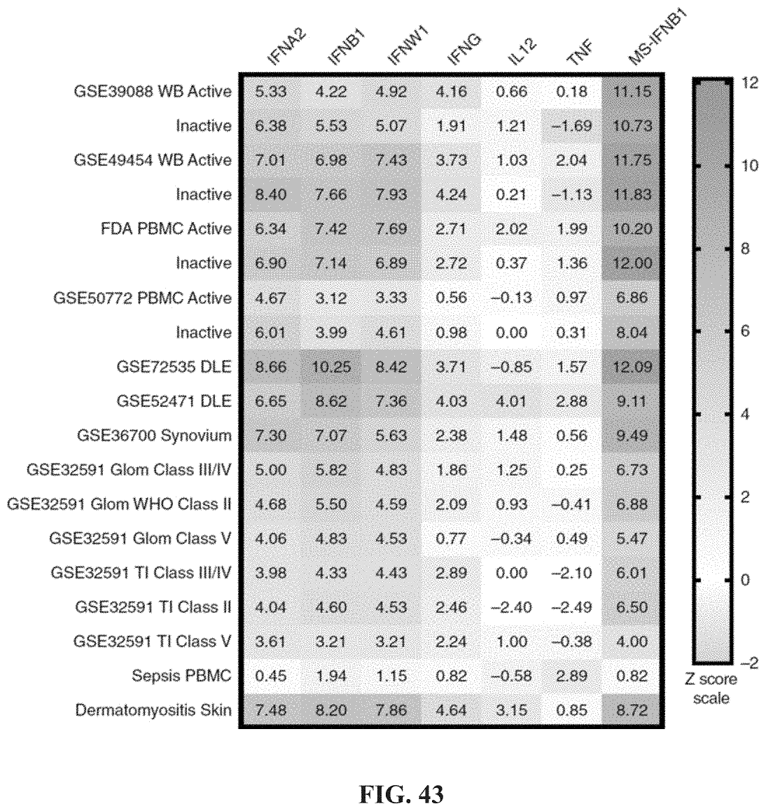

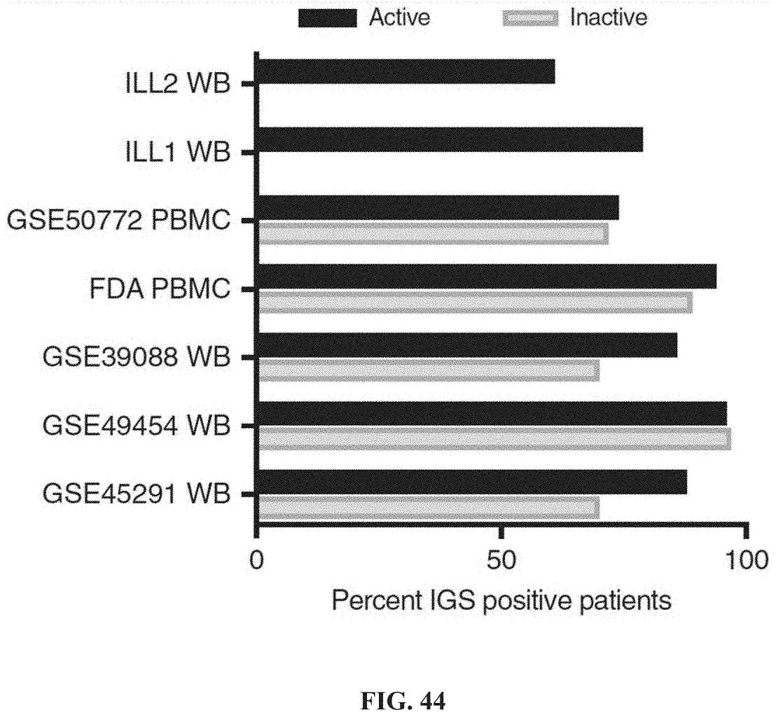

FIG. 43 demonstrated Z scores greater than six using the MS-IFNB1 signature for all SLE WB, PBMC, and SLE affected tissue datasets establishing both a high overlap and shared directionality of transcripts. The Z score for the control sepsis dataset using the MS-IFNB1 signature was not significant (Z=0.82), whereas the control dermatomyositis dataset was highly significant (Z=8.72).

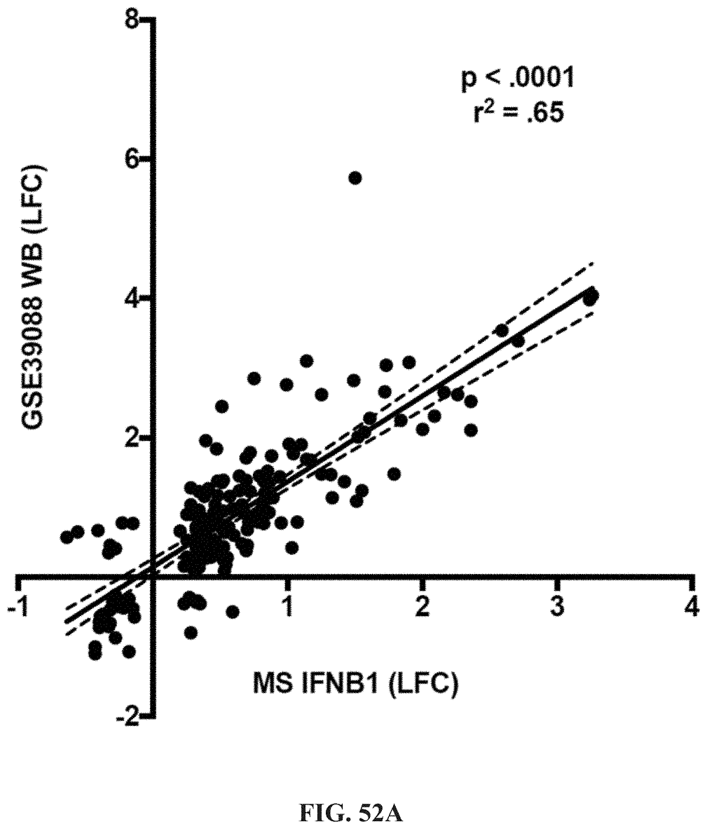

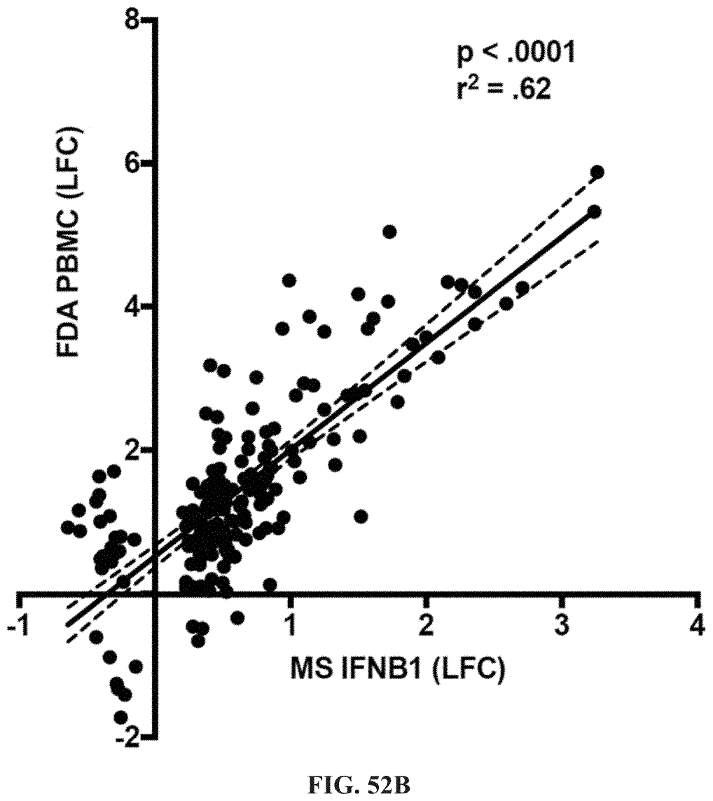

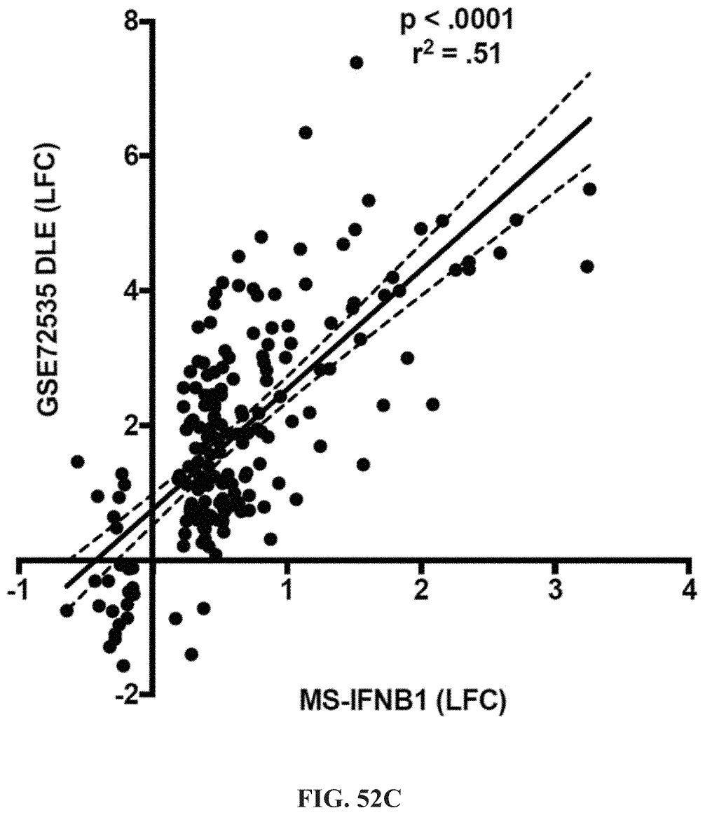

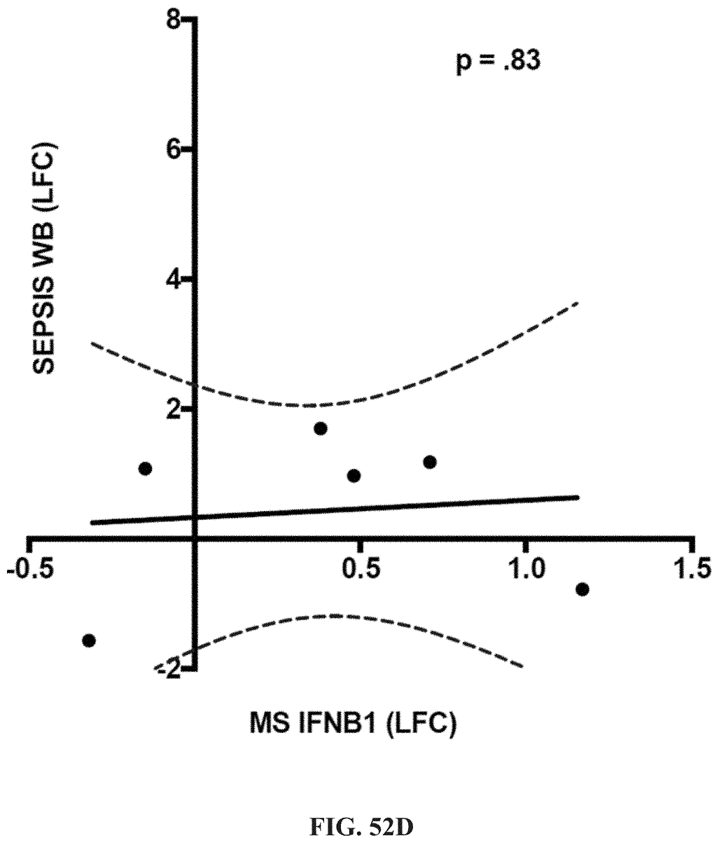

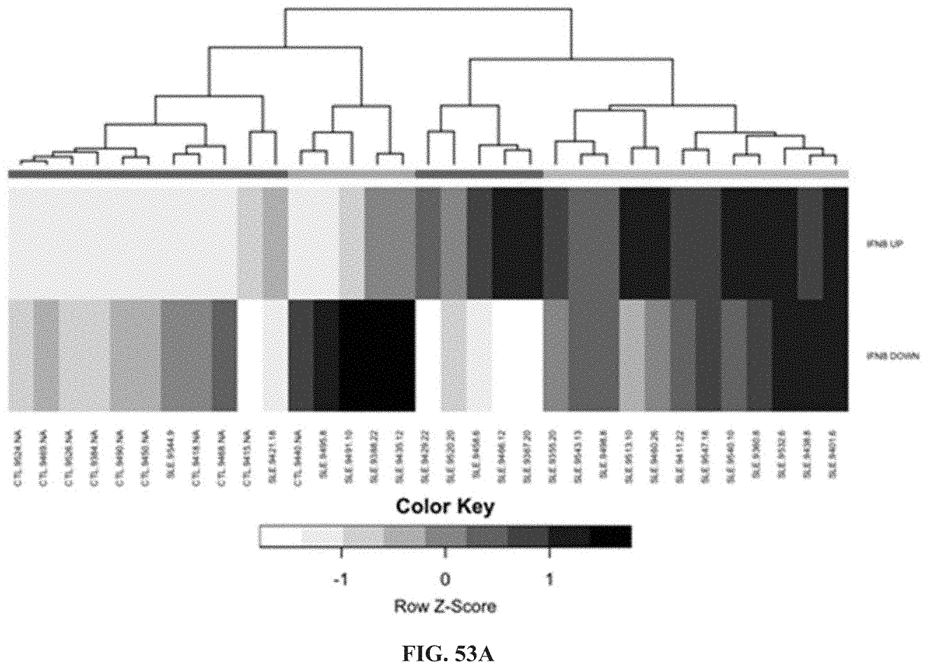

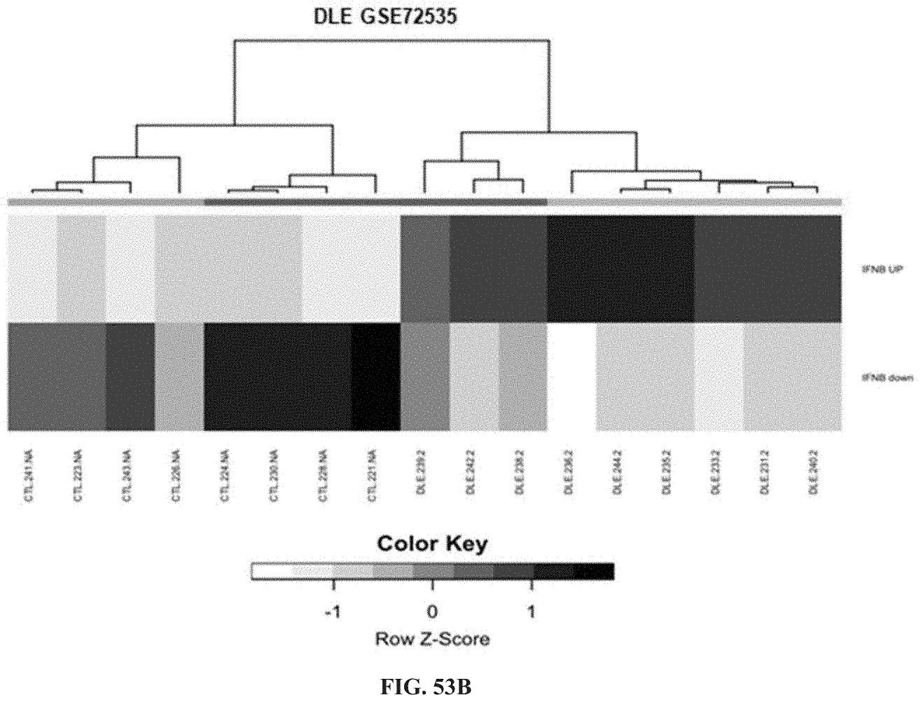

Confirmation of the high degree of overlap between the MS-IFNB1 signature and the SLE datasets was demonstrated by the significant correlation (p<0.0001) by linear regression to SLE WB, PBMC, and DLE datasets with coefficient of determination (r²) values of 0.51-0.65 (FIGS. 52A-52D). Additionally, both the increased and decreased transcripts for the MS-IFNB1 signature separated SLE cells and tissues from controls (FIGS. 53A-53B). The MS-IFNB1 Z scores were higher than all of the PBMC-derived Z scores, but IFNW1, IFNA2, and IFNB1 were still highly significant (Z>3) for all SLE WB, PBMC, and affected tissues, and for the control dermatomyositis dataset, but not the sepsis dataset. Similar to the results for GSVA enrichment, much higher scores were noted for the type I IGS in DLE and SLE synovium compared to LN Glom and TI. IFNG also had significant Z scores for all SLE affected tissues, but generally several standard deviations lower than the type I IFN scores. IL12 had a Z score above 2 for one DLE dataset and TNF had significant Z scores for one DLE and one WB SLE dataset; the highest Z score for TNF was obtained with the control sepsis dataset. Interestingly, Z scores were similar for SLE WB and PBMC datasets derived from active (SLEDAI≥6) and inactive (SLEDAI<6) patients for all the IGS tested.

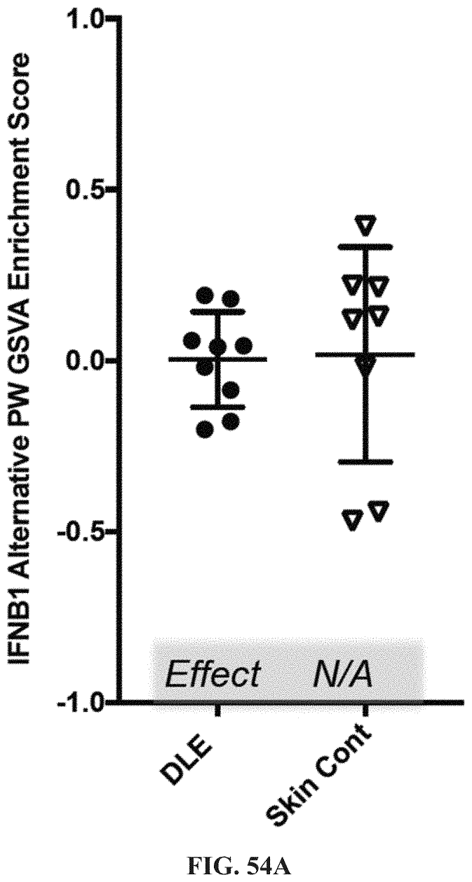

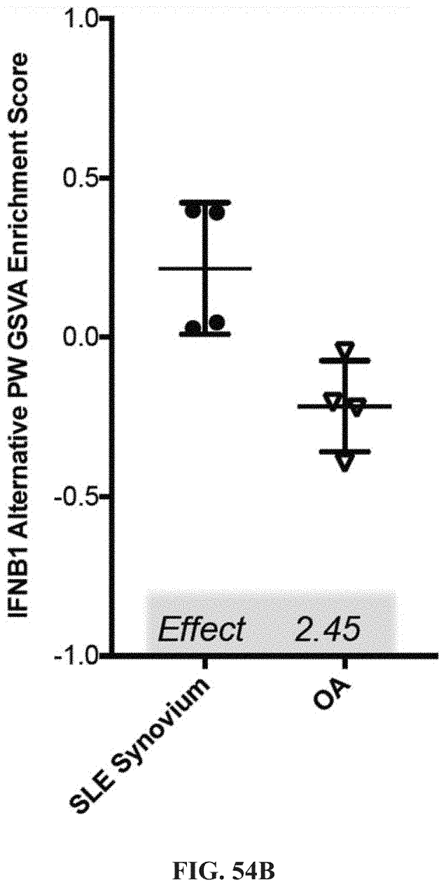

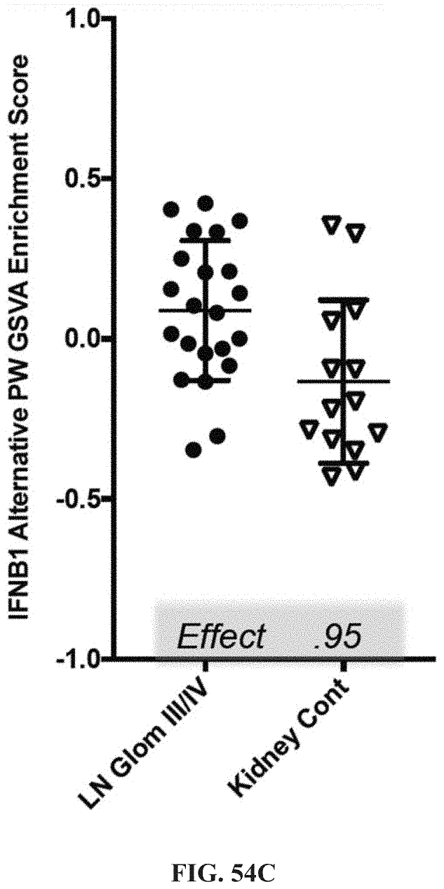

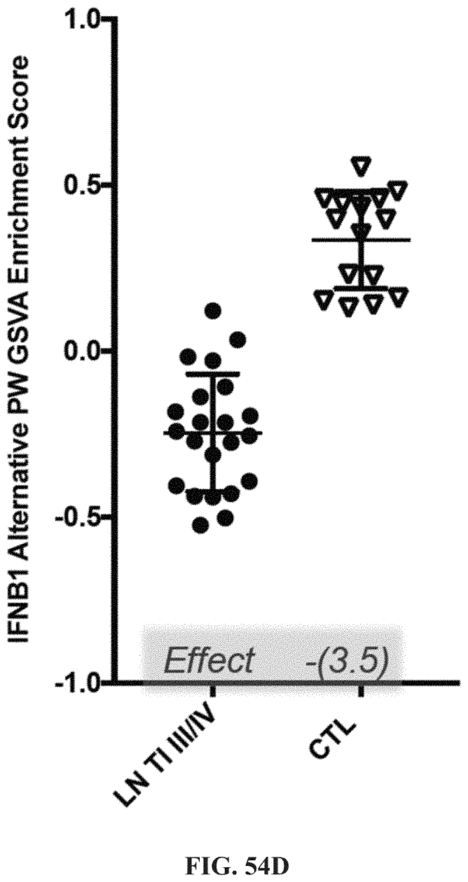

The alternative IFNB1 downstream signaling gene expression signature induced by IFNB1 binding only to IFNAR1 was taken from an experiment in which IFNAR2 deficient mouse cells were treated with IFNB1 and compared to untreated cells. The increased transcripts (Table 30) were used as a GSVA module to determine whether there was alternative IFNB1 signaling in SLE affected tissues. GSVA enrichment scores for SLE patients relative to controls showed low enrichment in SLE synovium (p=0.02, g=2.45), and LN glom class III/IV (p=0.01, g=0.95) and no enrichment in DLE or LN TI class III/IV (FIGS. 54A-54D). Taken together, both GSVA and Z score calculations suggest canonical, but not alternative, IFNB1 downstream signatures are strongly enriched in SLE PBMC, WB, skin, and synovium, and this downstream signature along with the IFNA2 and IFNW1 signatures are less prominent in LN.

The genes listed in Table 30 are described by, for example, [de Weerd, N. A. et al. Structural basis of a unique interferon-beta signaling axis mediated via the receptor IFNAR1. Nat. Immunol. 14, 901-907 (2013)], which is hereby incorporated by reference in its entirety; gene symbols are converted from mouse to human gene symbols.

TABLE 30
Genes of IFNB1 Alternative PW Increased Transcripts

| | | | | | | | | | |
|---|---|---|---|---|---|---|---|---|---|
| ACKR3 | CCL3L3 | CTPS1 | FMNL2 | IFI16 | MT1A | POGK | SERPINB2 | SOCS3 | TREML4 |
| ACOD1 | CCL4 | CXCL2 | FPR2 | IKBKE | MT2A | PPRC1 | SERPINE1 | SOD2 | TRIM13 |
| ADORA2B | CCL7 | CXCL3 | GFPT1 | IL1A | MYBPC2 | PROK2 | SHMT1 | SUSD6 | TRMT61A |
| AEN | CCRL2 | DNMT3L | Gk | IL1B | NAB2 | PTGES | SLAMF8 | TFEC | TXNRD1 |
| AMBP | CD14 | DOT1L | GPR84 | IL1RN | NOCT | PTGIR | SLC12A4 | TFRC | URB2 |
| ARG1 | CD207 | DRAM1 | GRWD1 | IL6 | NOP16 | PVR | SLC15A3 | TGM2 | ZNF503 |
| ARG2 | CD86 | DUSP16 | HBEGF | IRAK3 | NOP2 | RAB20 | SLC16A10 | TIMM8A | |
| ARHGAP31 | CDC42EP2 | EEF1E1 | HCAR2 | ITGA5 | NR1H3 | RAI14 | SLC20A1 | TLNRD1 | |
| ASPA | CDKN1A | EPHA4 | HEATR1 | ITGAX | NR4A1 | RELB | SLC25A25 | TLR2 | |
| B4GALT5 | CDR2 | ETS2 | HIVEP3 | KPNA2 | OLR1 | RND1 | SLC25A33 | TMA16 | |
| BCL3 | CLEC4E | EXOC3L4 | HSPA1A | MAFF | OSM | RRP12 | SLC2A6 | TNF | |
| BYSL | CLEC6A | F3 | HSPA1B | MARCKSL1 | PDIA6 | RRS1 | SLC39A14 | TNFAIP2 | |
| CA13 | COQ10B | FAM20C | ICAM1 | MARCO | PHLDA1 | SAA1 | SLC7A2 | TNFAIP3 | |
| CAMKK2 | CRYAA | FCRL5 | ID1 | MEFV | PHLDB1 | SCAMP1 | SLC7A5 | TNFSF14 | |
| CCL2 | CSTA | FFAR2 | IER3 | MMP14 | PIM1 | SDC4 | SNX18 | TREM1 | |

### Example 15: An IGS is Readily Detectable in SLE Patients with Low Disease Activity

The similar Z score calculations in active and inactive SLE WB and PBMC (FIG. 4) suggested the IGS was expressed equivalently in active and inactive SLE patients. In order to determine the relationship between the IGS and SLE disease severity, five SLE WB and two SLE PBMC datasets were separated into active (SLEDAI≥6) and inactive (SLEDAI<6) patients.

A mean of 73% of active SLE patients and a mean of 66% of inactive SLE patients expressed the IFN core signature (FIG. 5). The IFNA2, IFNB1, IFNW1, MS-IFNB1, and HepC-IFNA2 signatures yielded similar results. To further assess the relationship between the IGS and SLE disease activity, WGCNA was carried out on four WB and two PBMC SLE datasets, and each dataset yielded one module comprising IGS genes. Pearson correlation of each IFN module eigengene to the presence of SLE disease was significant (p<0.0005) and positive for all datasets (range of r=0.16 to 0.79), but the magnitude of the correlation to disease activity measured by SLEDAI was low and variable (range of r=−0.49 to 0.37) even though some of the relationships to SLEDAI were significant (p<0.05).

### Example 16: An IGS Varies Over Time in 30% of SLE Patients in Association with Significant Changes in Cell Populations

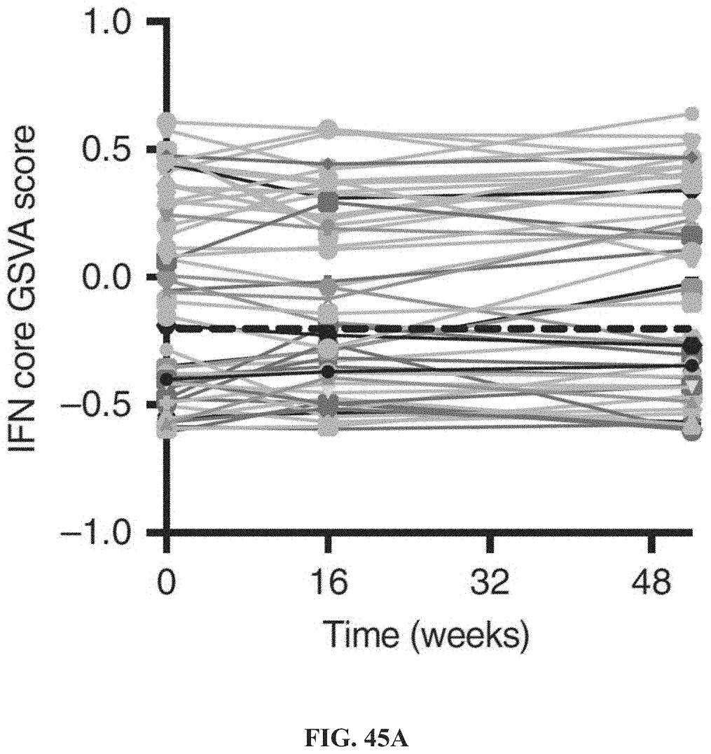

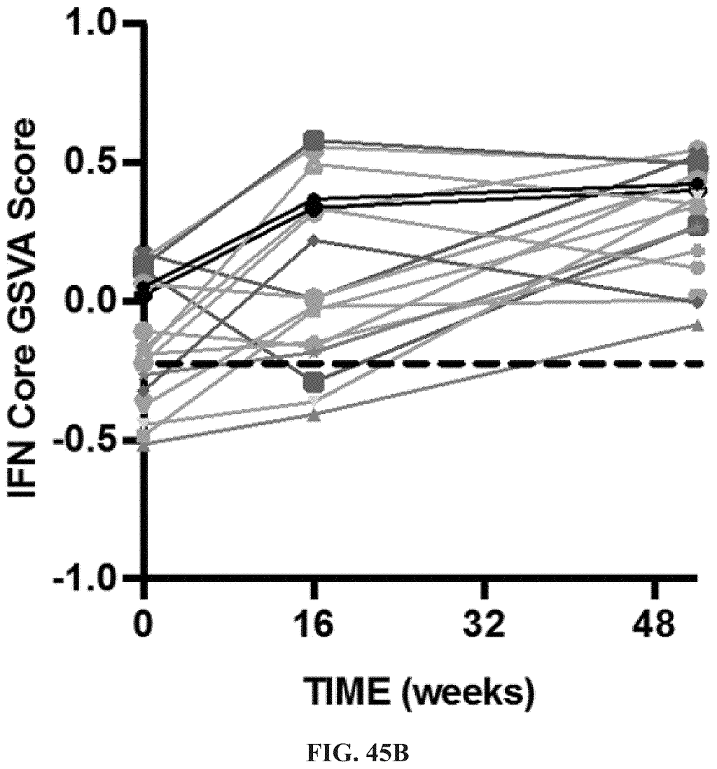

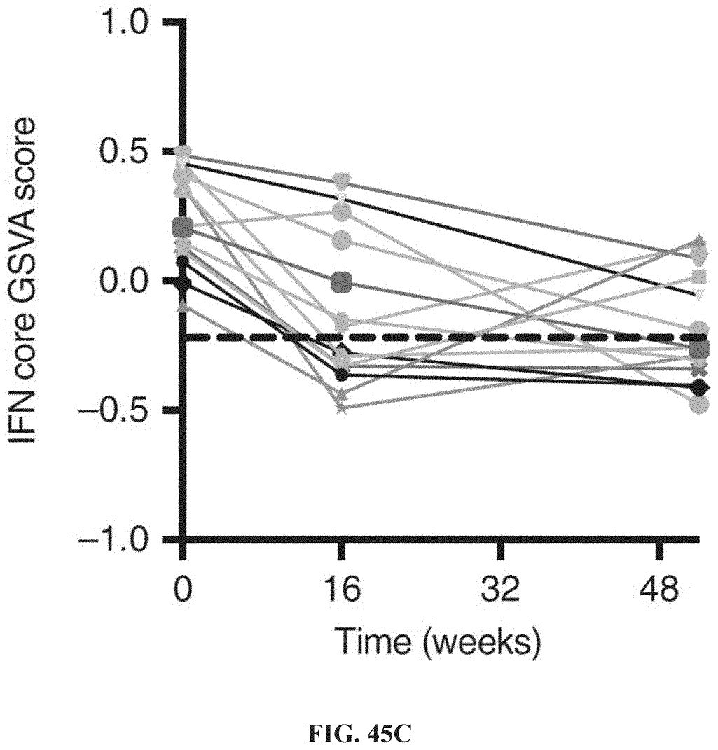

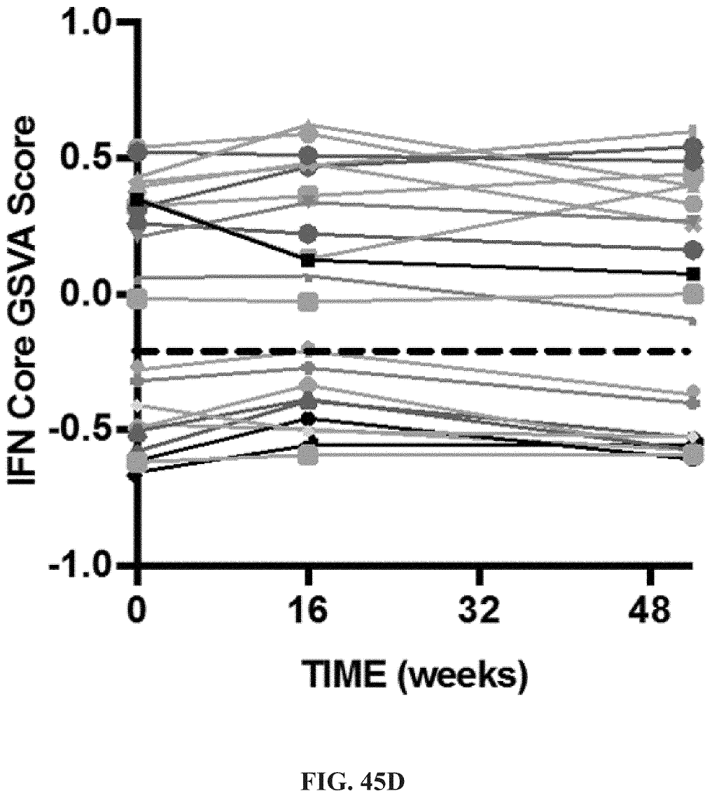

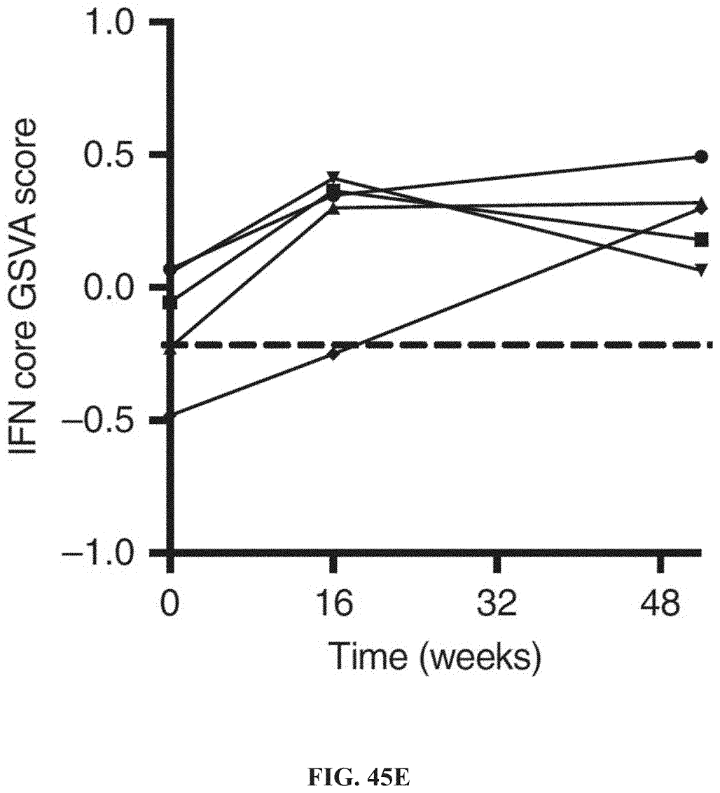

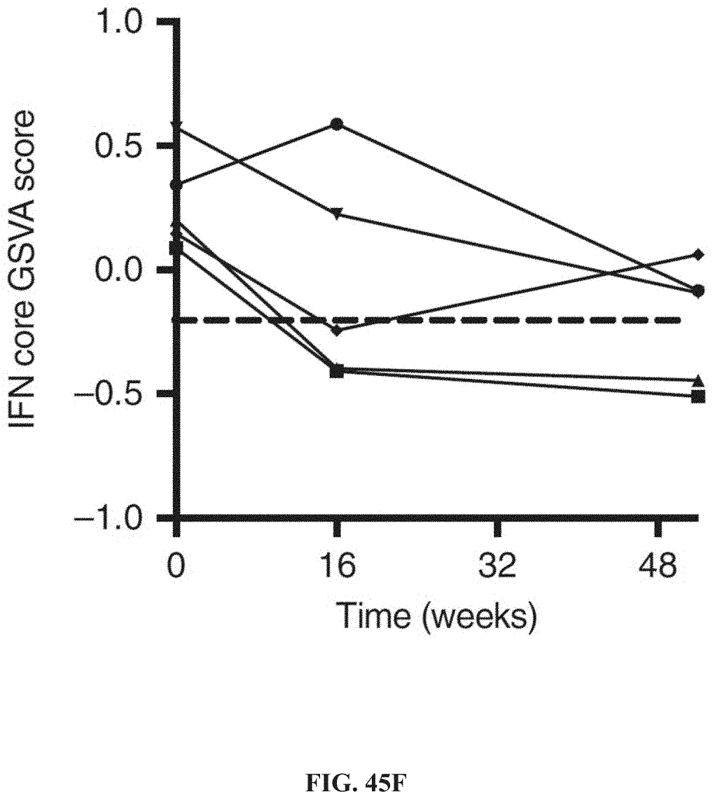

Time course experiments were analyzed to determine whether SLE patients gain or lose the IGS over time. IFN GSVA scores were calculated for SLE patients on standard-of-care (SOC) treatment at three time points: baseline, 16 weeks and 52 weeks. For the GSE88885 dataset, 60% of subjects expressed an IFN core signature at baseline and 62% (53 patients) had only non-significant changes (SD<0.2) in their IFN core GSVA scores over one year, whereas 38% (33 patients) had significant changes in their IFN core enrichment scores (SD>0.2) (FIG. 45A). 18 patients went from a negative to a positive IGS (FIG. 45B), and 15 patients went from a positive to a negative IGS (FIG. 45C). In the GSE88886 time course dataset, similar changes were noted; 64% of subjects had an IFN core signature at baseline and 70% (23 patients) had only non-significant changes (SD<0.2) in their IFN core GSVA scores, whereas 30% (10 patients) had significant (SD>0.2) changes in their IFN core GSVA scores (FIG. 45D). Five patients went from a positive to a negative IGS, and five patients went from a negative to positive IGS (FIGS. 45E-45F). The IFNA2, IFNB1, IFNW1, MS-IFNB1, and HepC-IFNA2 signatures showed similar patterns of change.

To understand the relationship between the IGS and SLEDAI over time, analysis was carried out on a time course microarray experiment of WB from ten SLE patients with active LN. Samples were taken before therapy (t=0), 12 weeks after treatment with high-dose immunosuppressives (t=12), and after 12 more weeks of moderate- to low-dose immunosuppressive therapy (t=24). Nine out of ten patients had changes in their IFN core GSVA scores by 24 weeks (SD>0.26; range: 0.26-0.54) whereas one subject had no change in the IGS enrichment score over time (SD=0.02).

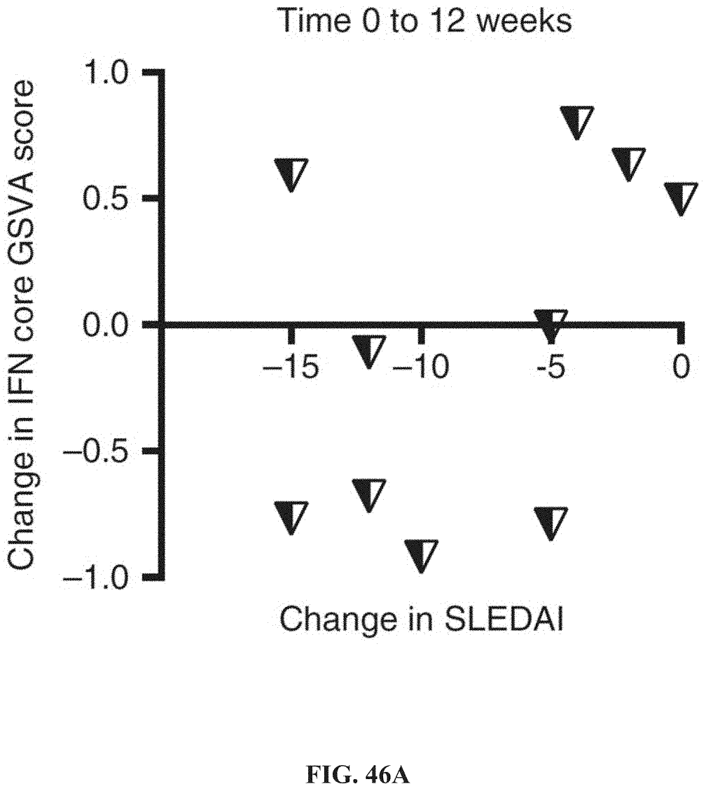

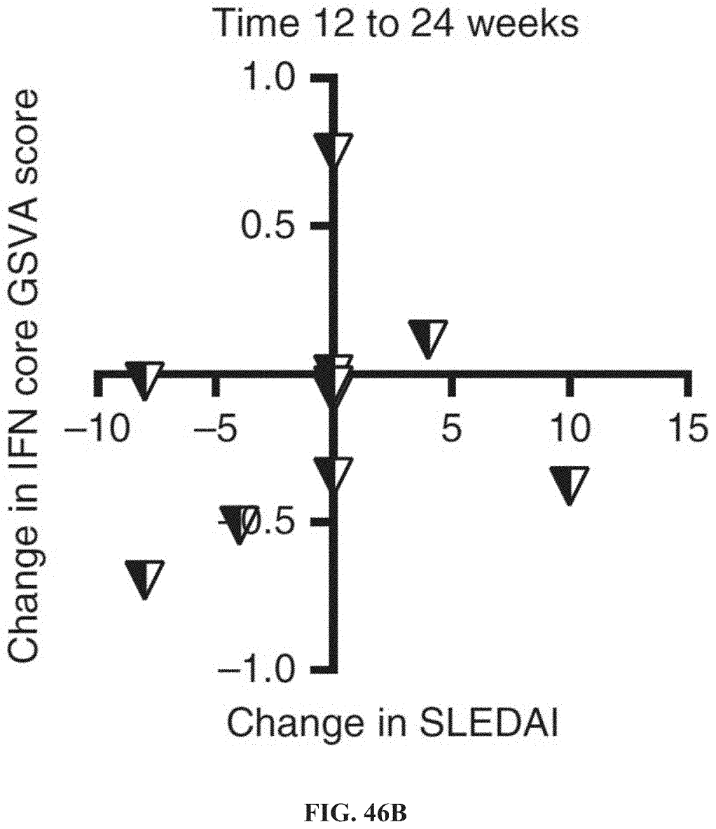

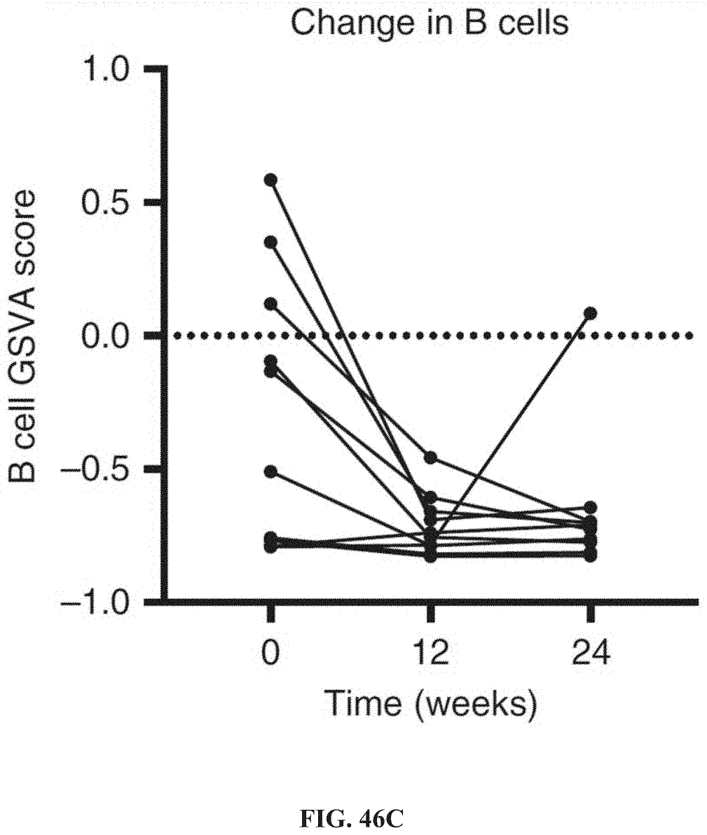

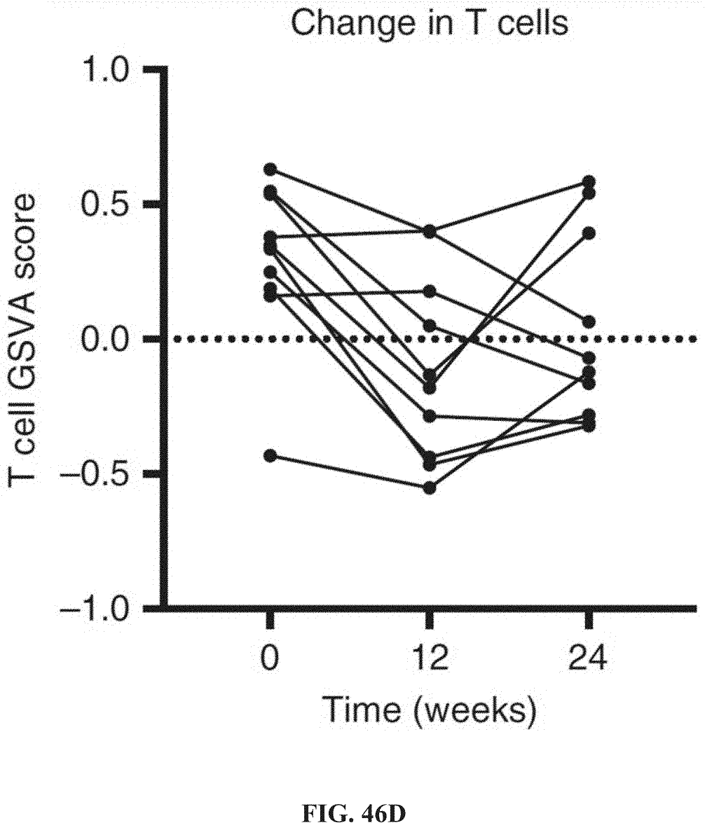

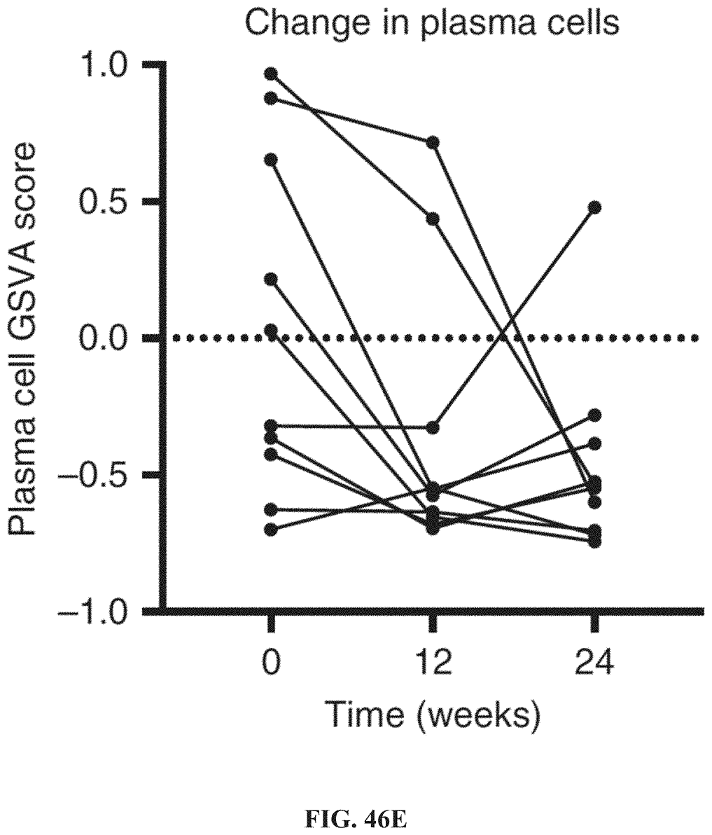

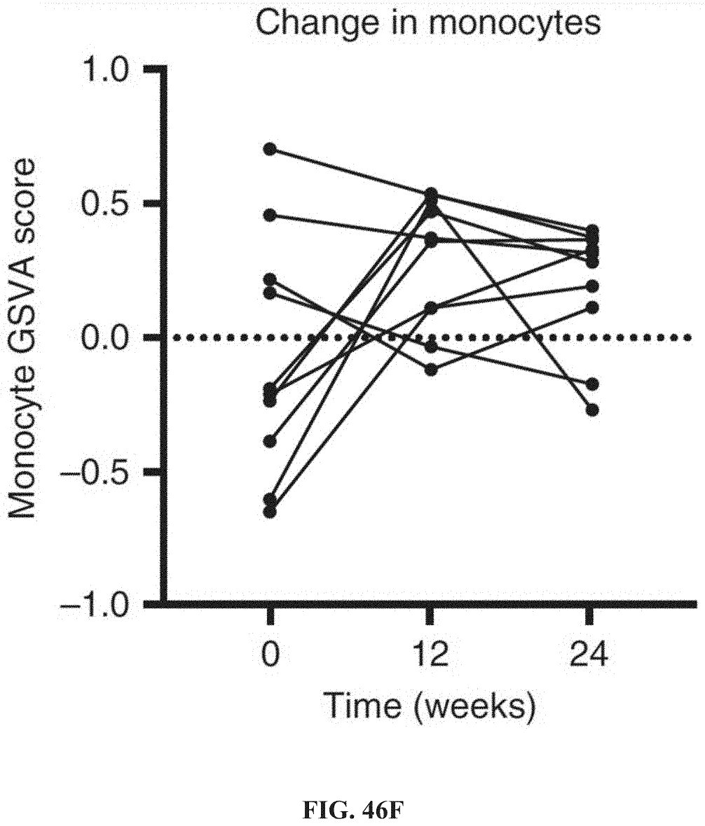

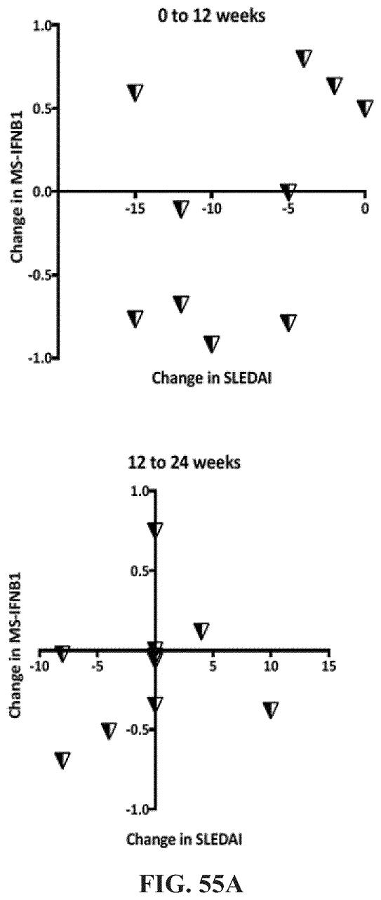

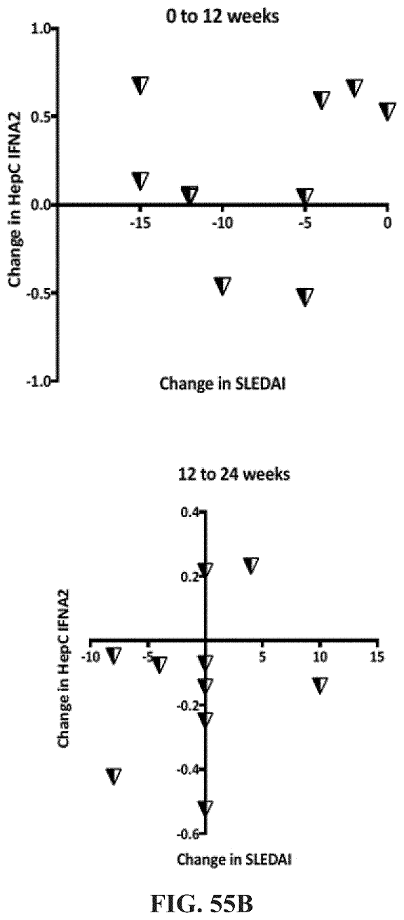

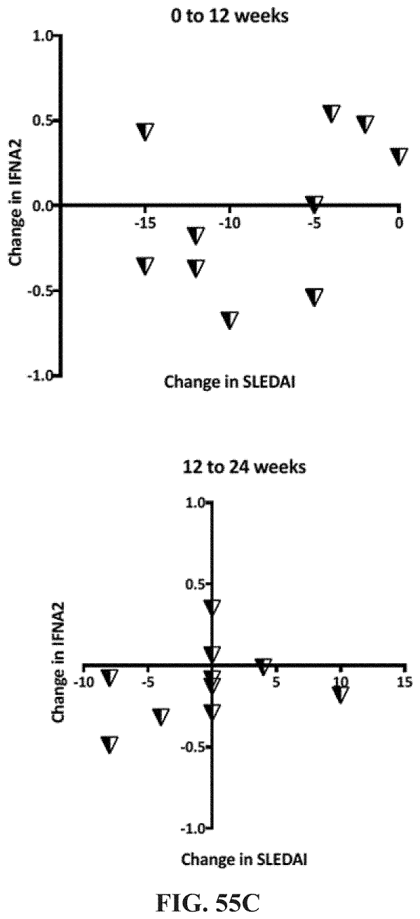

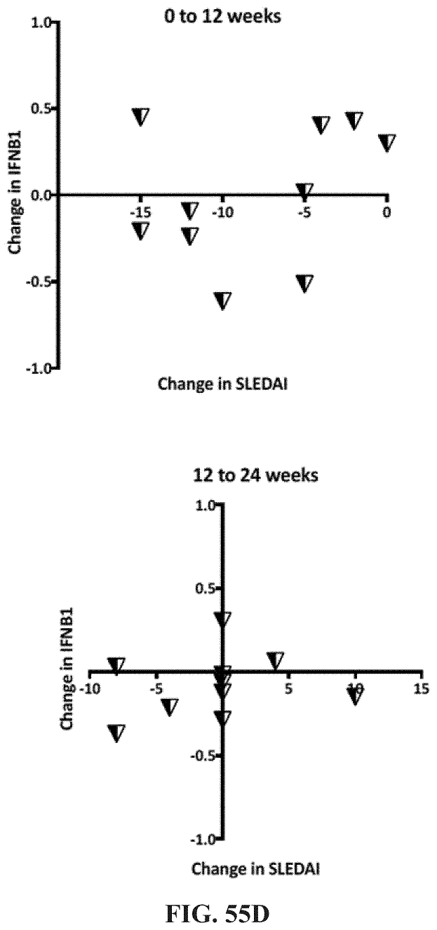

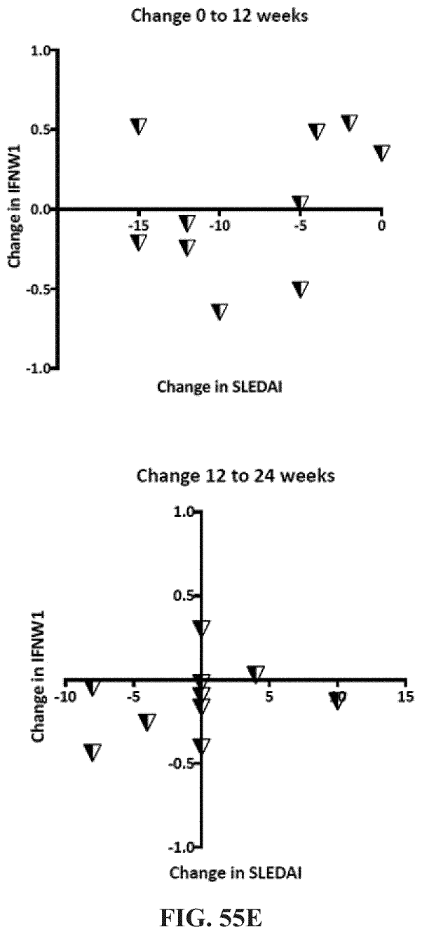

FIGS. 46A-46F show the change in SLEDAI versus the change in the IFN core GSVA score between 0 to 12 weeks (FIG. 46A) and 12 to 24 weeks (FIG. 46B); the other IGS gave similar results and are shown in FIGS. 55A-55E. Of the nine patients with decreases in SLEDAI from zero to 12 weeks, three had increases in IGS, four had decreases, and two patients had no change in their IGS. Likewise, between 12 and 24 weeks no consistent pattern for the change in SLEDAI versus the change in IGS was detected. GSVA enrichment scores using gene signatures for T cells, B cells, and monocytes (generated as described in the methods) showed significant changes between 0 and 12 weeks (ANOVA p<0.05) and there was a relative depletion of plasma cells that was not significant (FIGS. 46C-46F). These results demonstrated that at least 30% of SLE patients on SOC may have a significant change in their IGS over time, and that changes in cell populations because of immunosuppressive therapy may significantly affect the IFN signature, but there is no association between the IGS and the SLEDAI.

### Example 17: An IGS Correlates with a Monocyte Signature in the Peripheral Blood

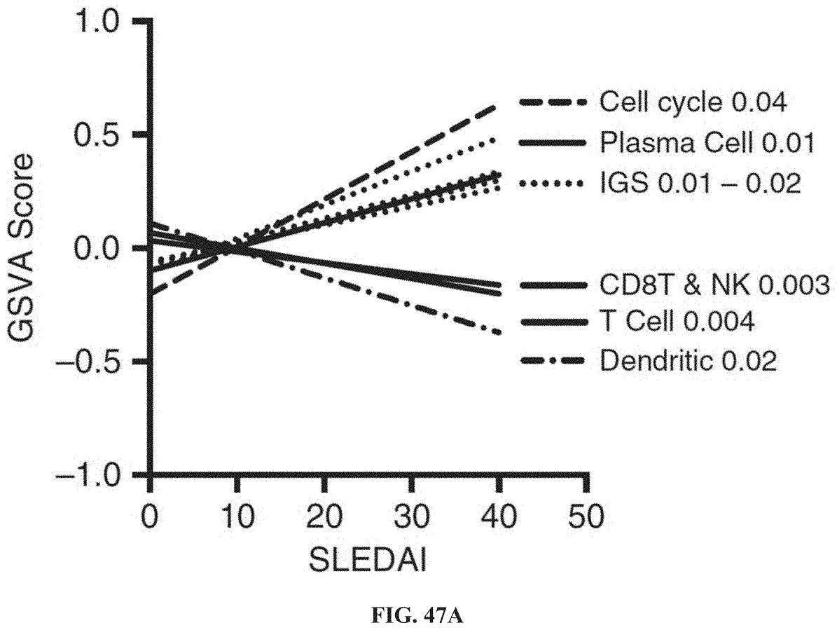

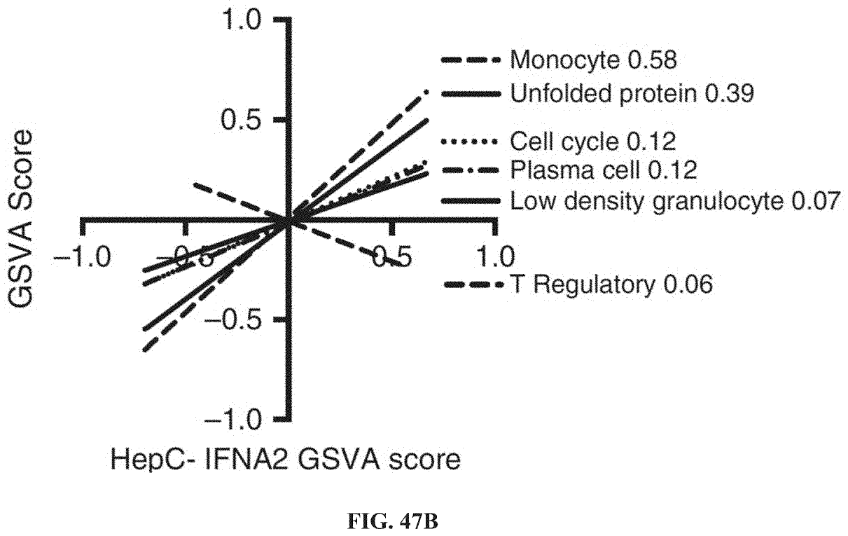

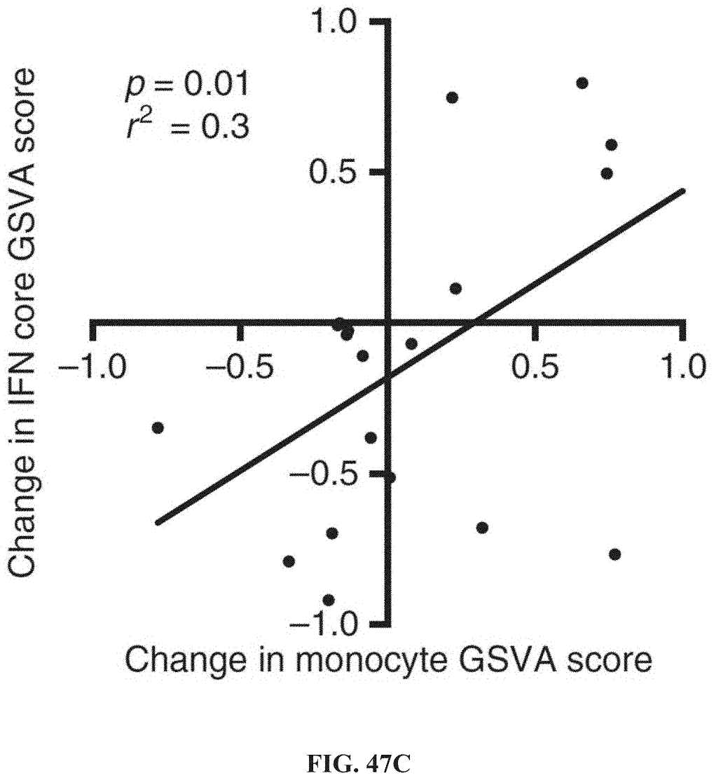

Linear regression analysis was used to determine the relationship between the IGS and cell types, cellular processes, clinical measures and SLEDAI. For individual datasets, the most consistent positive relationship with SLEDAI (a non-zero slope; p<0.05) was with genes involved in regulation of the cell cycle (r² range: 0.02-0.18). Plasma cells (r²: 0.01-0.17), ds DNA (r²: 0.06-0.21), IFN core (r²: 0.07-0.14), and IFNB1 (r²: 0.01-0.29) also had a positive relationship (p<0.05) with SLEDAI, but the r² predictive values were low. T cell, CD8-NK-NKT (natural killer T) cell, and dendritic cell GSVA enrichment scores had significant negative relationships with SLEDAI (p<0.05) in most datasets but also with low ranges of predictive r² values; T cell (0.09-0.321), CD8-NK-NKT (0.06-0.26) and dendritic cell (0.02-0.2) (FIG. 47A).

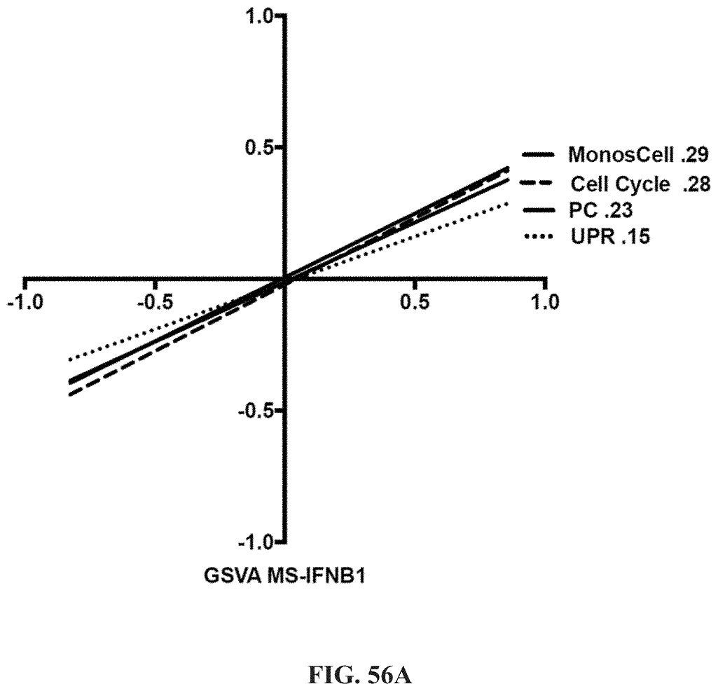

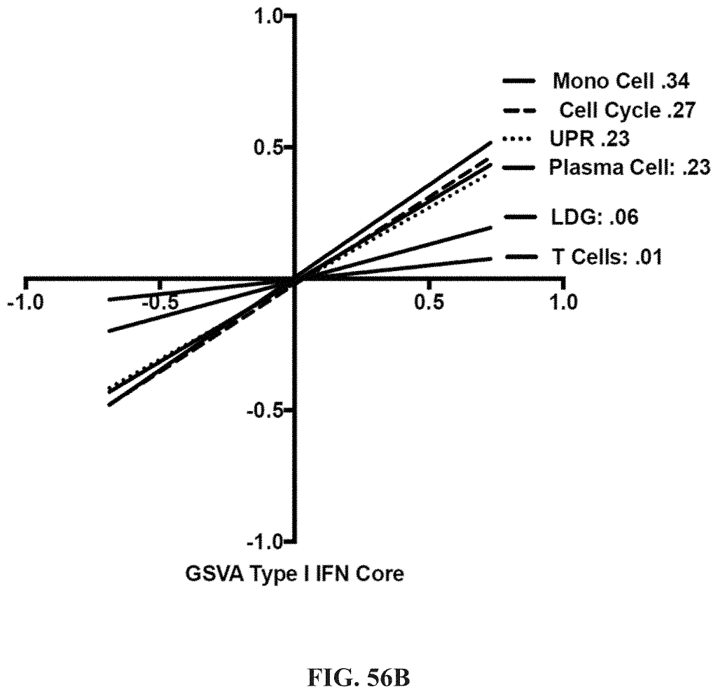

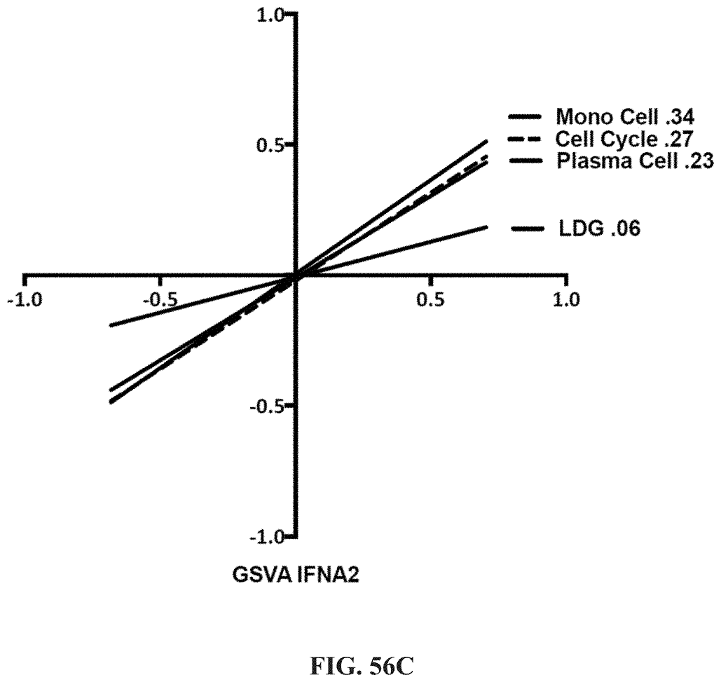

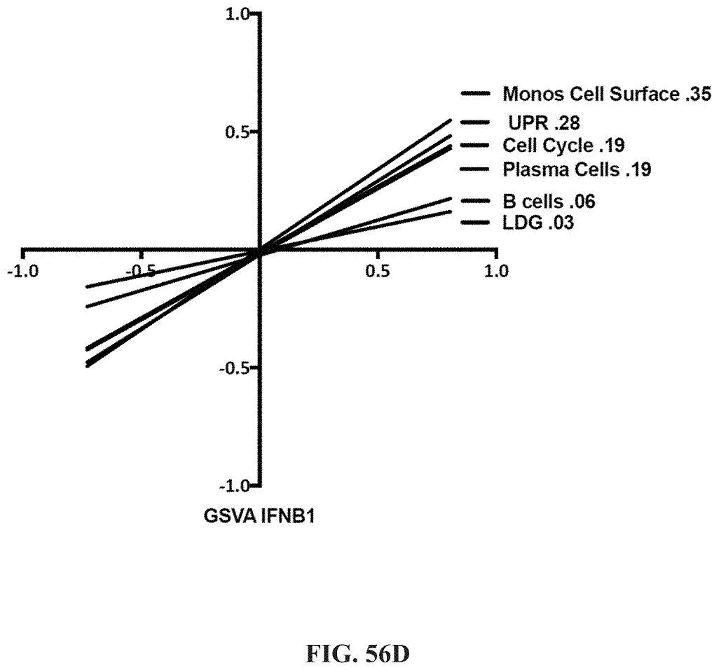

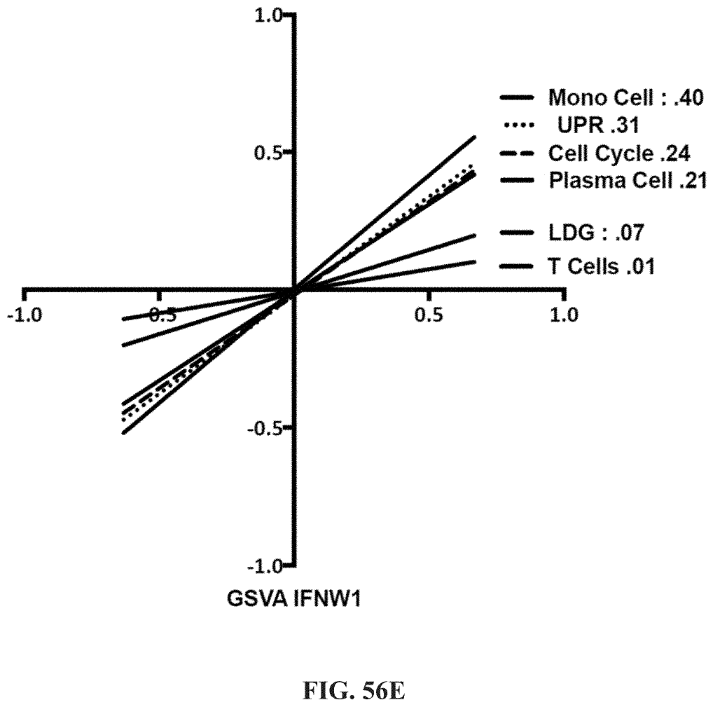

To determine whether the IGS detected in SLE patients was related to a specific type of hematopoietic cell or process, linear regression analysis was carried out between the GSVA enrichment scores for cell signatures and processes and the IGS in each SLE patient from ten SLE WB and PBMC datasets. Transcripts in common between cell type or process modules and the IGS were removed from the IGS before analysis so that genes in common did not contribute to the relationship between the GSVA enrichment scores. The strongest relationship to the IGS was to monocyte surface transcripts with a significant non-zero slope (p<0.0001) and a range of r² values of 0.29-0.58. Other categories with a significant relationship (p<0.05) to most IGS, but with a lower range of predictive values were the cell cycle (0.12-0.28), plasma cells (0.12-0.23), the unfolded protein response (UPR; 0.15-0.39), low density granulocytes (LDGs 0.03-0.07), and anti-ds DNA levels (0.02-0.09) (FIGS. 47B and 56A-56E; Table 31). For T cell categories, NKT cells and dendritic cells, datasets with fewer patients had negative relationships to the IFN signatures, but the two largest datasets (GSE88884 ILL1 and ILL2) had low, positive relationships with the IFN signatures. The association of the monocyte signature with the IGS suggested that monocytes and the IGS would change synchronously. In order to test this, the change in monocytes versus the change in the IFN core signature for time-course dataset GSE72747 described in FIGS. 46A-46F was carried out. FIG. 47C demonstrated a significant relationship (r²=0.3; p<0.05) by linear regression analysis between the change in monocytes and the change in IFN core signature.

TABLE 31
Linear Regression r² Values for IFNs and Cell Type Signatures

| | | | | Mono | | | | | | |
|---|---|---|---|---|---|---|---|---|---|---|
| | Cell | Plasma | | Cell | | B | T | T | CD8 T | Anergic |
| | Cycle | Cells | UPR | Surfac | LDG | Cells | Reg | Cells | NKT | T |
| MS-IFNB1 | 0.28 | 0.23 | 0.15 | 0.29 | | | | | | |
| HepC-IFNA2 | 0.12 | 0.12 | 0.39 | 0.58 | 0.07 | | 0.05 | 0.01 | 0.02 | 0.02 |
| IFNA2 | 0.27 | 0.23 | | 0.34 | 0.04 | | | | | |
| IFNB1 | 0.19 | 0.19 | 0.28 | 0.35 | 0.03 | 0.06 | | | | |
| IFNW1 | 0.24 | 0.04 | 0.31 | 0.40 | 0.05 | | | 0.01 | | |
| IFN Core | 0.27 | 0.23 | 0.24 | 0.34 | 0.04 | | | 0.01 | | |

Insufficient plasmacytoid dendritic cell (pDC) specific transcripts made GSVA unreliable, but pDC specific transcripts CLEC4C (BDCA-2) and NRP1 (BDCA-4) were decreased in 6/10 and 2/10 SLE WB and PBMC datasets respectively, and not uniformly associated with the IGS).

The strong relationship of the IGS to the monocyte signature could be related to a stronger relative IGS expression in monocytes compared to B and T cells. In order to test this, DE analysis was performed between CD4 T cells, CD19 B cells and CD14 monocytes from active (SLEDAI≥6) SLE patients obtained from publicly available microarray datasets. Comparison of expression of the nonoverlapping IFN core signature transcripts revealed that monocytes overexpressed (LFC>1, FDR<0.05) three times as many IGS transcripts as T cells (92 transcripts to 28 transcripts) or B cells (94 transcripts to 29 transcripts). Transcripts increased by more than LFC=4 in SLE monocytes compared to both T cells and B cells included IL1RN, SERPING1, PLSCR1, EIF2AK2, JAK2, and CXCL10. LAMPS was overexpressed in SLE T cells compared to SLE monocytes and B cells and APOBEC3B, STAP1 and SPIB were overexpressed in SLE B cells compared to SLE monocytes and T cells. Both T cells and B cells overexpressed DUSP5, CCND2, RGS1, CAD, ISG20, SOCS1, SIT1 and SP140 compared to SLE monocytes. The IGS transcripts not DE between purified cells were also of interest; IFI27 was frequently the most overexpressed IGS transcript in SLE datasets, but it was not DE between T cells, B cells and monocytes. Additionally, IFI44L, IFIH1, IFIT3, OASL, RSAD2, SPATS2L and USP18 were all highly DE when each cell type was compared to controls, but not DE between SLE T cells, B cells or monocytes. Comparison of these eight genes to the individual signatures used in FIGS. 47A-47C showed that the HepC-IFNA2 signature had none of these transcripts and the MS-IFNB1 signature had all eight which may explain in part the weaker predictive relationship (r²=0.29) between the MS-IFNB1 signature and monocytes.

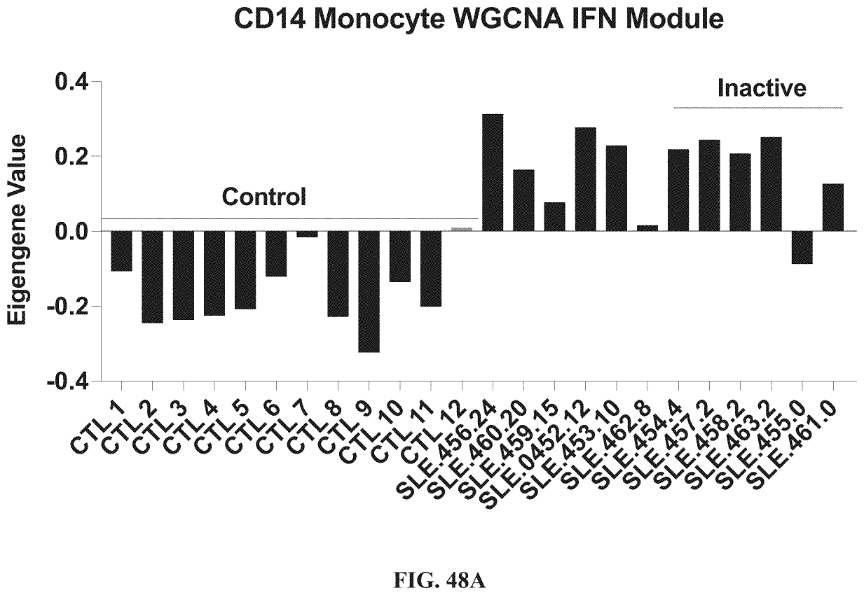

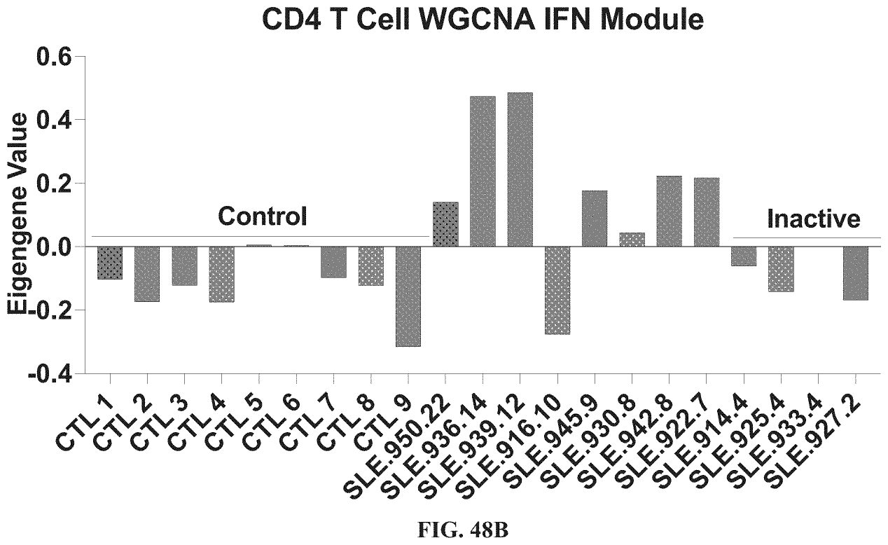

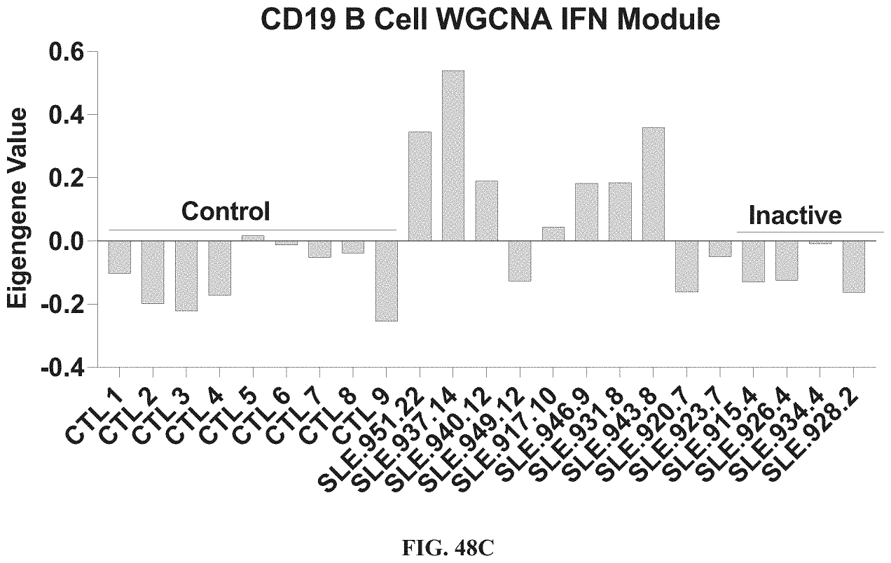

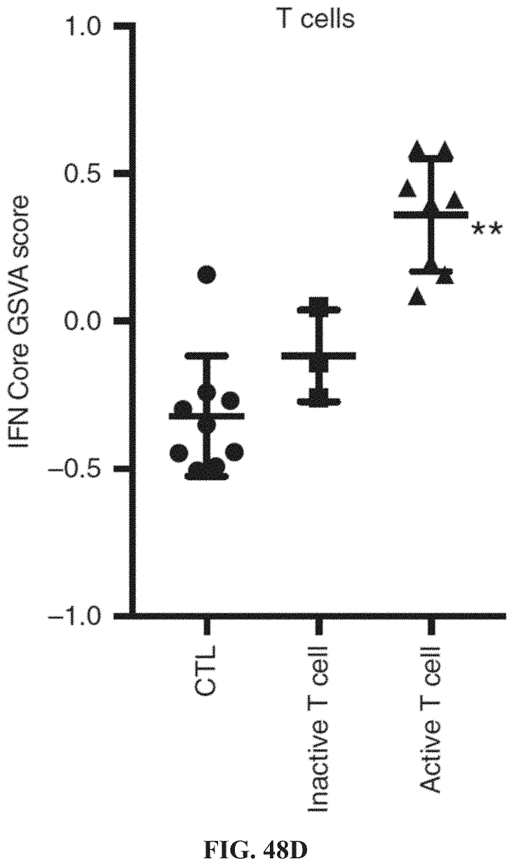

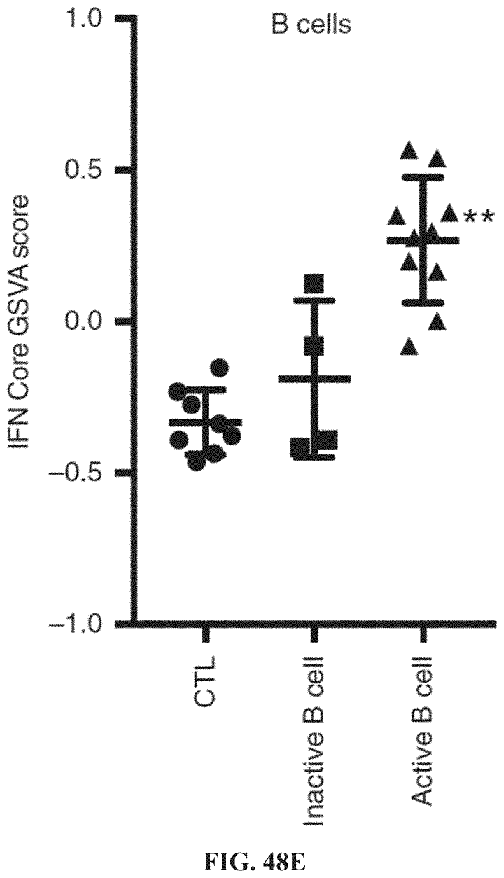

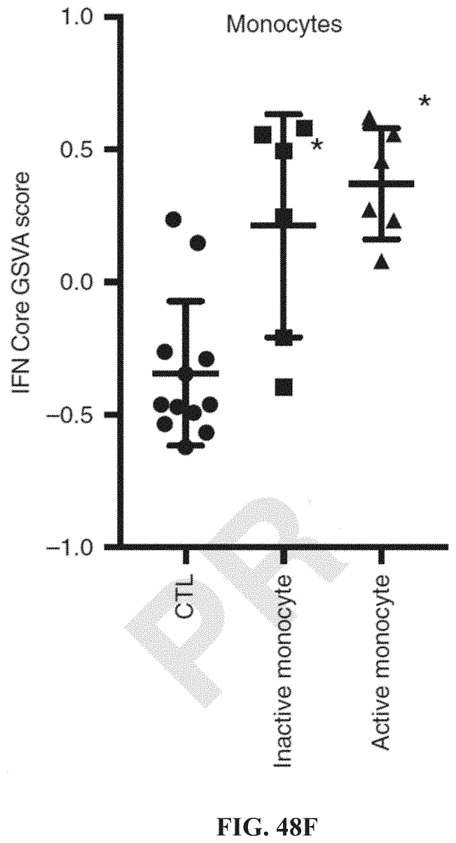

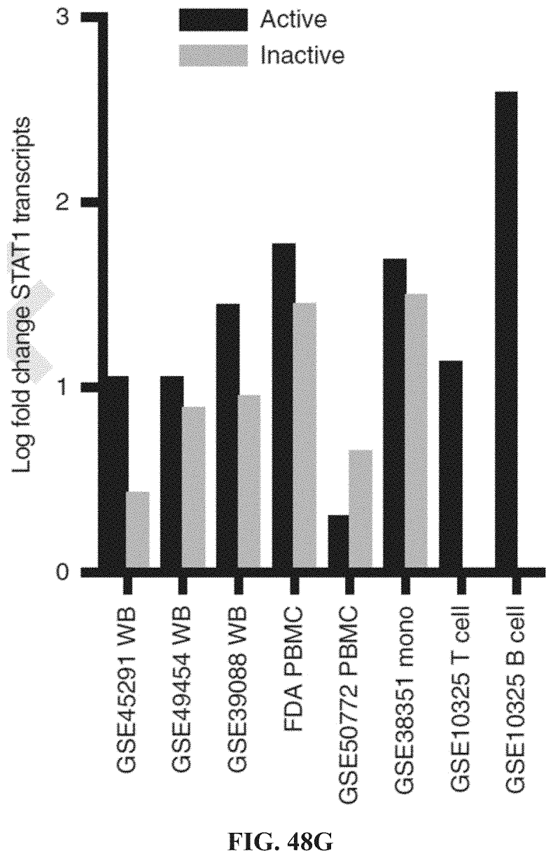

To explore the relationship between monocytes and the IGS in greater detail, WGCNA analysis was carried out on purified CD14 monocytes, CD4 T cells and CD19 B cells from SLE patients with active and inactive disease. A discrete IGS module was delineated for monocytes and T cells and the IGS in CD19 B cells grouped with cell cycle transcripts (FIGS. 48A-48C). All three modules showed significant Pearson correlation (p<0.05; r≥0.5) to the presence of SLE (versus control) but only the IGS modules from T cells and B cells also showed significant correlation to SLEDAI (p<0.05; r≥0.5). Eigengene values for monocytes from SLE patients with inactive disease had mostly positive values for the IFN module in contrast to T cells and B cells from SLE patients with inactive disease who exhibited negative values. GSVA enrichment scores using the IFN core signature showed that T cells (FIG. 48D) and B cells (FIG. 48E) isolated from inactive SLE patients displayed low or absent IGS, whereas monocytes from inactive SLE patients had IGS similar to monocytes from active SLE patients (FIG. 48F). As IFNs have been shown to induce the transcription of STAT1 in monocytes and increased transcription could lead to an increase in unphosphorylated STAT1 (U-Stat1) and a prolonged IGS in the absence of IFNs transcripts for STAT1 were evaluated and shown to be elevated in both active and inactive SLE WB, PBMC and monocyte datasets, but not in T cells and B cells from inactive SLE patients (FIG. 48G). Thus, monocytes in WB and PBMC may retain the IGS in SLE patients with low disease activity, and also relatively over-express more IGS transcripts than T cells or B cells.

### Example 18: Interferon Modules Generated from Stimulated PBMCs, WB of IFNA-Treated HepC Patients, and WB of IFNB-Treated MS Patients

Using systems and methods provided herein, specific interferon modules were generated (i) for IFNA2, IFNB1, IFNG, and IFNW from stimulated PBMCs (peripheral blood mononuclear cells), (ii) from WB of IFNA2-treated patients with hepatitis C (HepC-IFNA2), and (iii) from WB of patients with multiple sclerosis who are treated chronically with IFNB1 (MS-IFNB1). Responses to each of the interferons were measured, and specific sets of genes that are specific for the measured responses (for SLE patients versus healthy controls) are shown in Table 32.

TABLE 32
Genes with Induced Transcripts in PBMC by IFNA2, IFNB1, IFNG, and
IFNW Treatment; in IFNA2-Treated HepC; and in IFNB1-treated MS

| IFNA2 | IFNB1 | IFNG | IFNW | | | | |
|---|---|---|---|---|---|---|---|
| PBMC | PBMC | PBMC | PBMC | HEPC-IFNA2 | | MS-IFNB1 | |
| TAF5L | IRF4 | P2RY13 | FGL2 | ACSL4 | RB1 | ACOT9 | RIN2 |
| RET | ADAM19 | CTNND2 | ERCC4 | ACTA2 | RNF44 | APOL6 | RSAD2 |
| CDK4 | ALOX5 | ICAM1 | MBNL1 | AIF1 | S100A12 | BATF2 | SAMD9 |
| CDC42EP1 | BAK1 | VSNL1 | MLF1 | AP1S2 | S100A8 | C19orf66 | SAMD9L |
| SYN2 | CCL4 | C4A | ABCB10 | CBX4 | S100A9 | CCL2 | SHISA5 |
| HLA-DRB5 | CXCL2 | SLC1A5 | BRD4 | CETN3 | SLC18B1 | CEACAM1 | SLFN12 |
| PGGT1B | DHFR | C1QB | FGF13 | CHSY1 | SNX6 | CHMP5 | SPATS2L |
| | FPR2 | ETAA1 | CD47 | CST3 | STAT3 | CMPK2 | TDRD7 |
| | HK2 | HP | SLC30A4 | CTNND1 | THEMIS2 | CNP | TMEM62 |
| | IKBKE | PCDH9 | SFT2D2 | CTSC | TNFSF13B | CSRNP1 | TRAFD1 |
| | ITGAX | FBLN1 | CASK | CUL1 | TOBI | CXorf21 | TRIM5 |
| | KCNMB1 | NLRP1 | TNFAIP3 | CYLD | TOP1 | DDX58 | TYMP |
| | LILRA1 | IL1A | CCR7 | DAPP1 | WSB1 | DDX60 | ZBP1 |
| | LTA | MMP25 | | DEK | ZFP36 | DTX3L | ZCCHC2 |
| | LTB4R | CPT1B | | FPR1 | | ETV7 | |
| | NAPSA | PLA2G4C | | GCA | | FAM46A | |
| | NBN | SPRY4 | | GLRX | | GALM | |
| | PDE4B | NET1 | | GNG10 | | GBP4 | |
| | PFKFB3 | UBD | | ITGAM | | GTPBP1 | |
| | PFKP | CLEC10A | | LCP2 | | HERC6 | |
| | PIM2 | LIMK2 | | LPAR6 | | HESX1 | |
| | RASGRP1 | | | LPIN2 | | IFIT2 | |
| | RIPK1 | | | MAF | | LGALS9C | |
| | RNF114 | | | MTHFD2 | | MOV10 | |
| | RRBP1 | | | NRGN | | MT2A | |
| | STOML2 | | | PHF11 | | NEXN | |
| | TANK | | | PIM1 | | OASL | |
| | TLR1 | | | PPA1 | | PARP12 | |
| | USP15 | | | PRPS2 | | PI4K2B | |
| | UVRAG | | | PSMA4 | | PIK3AP1 | |
| | SPTLC2 | | | RAB8B | | PNPT1 | |
| | ADAP2 | | | RARRES3 | | RAB8A | |

### Example 19: DE Analysis of LDG Genes

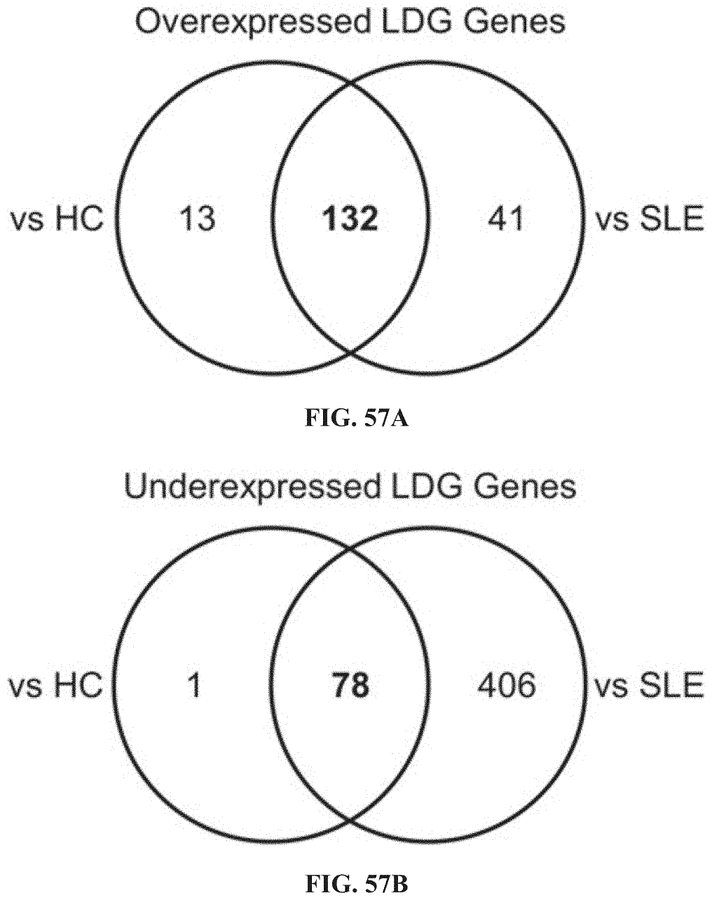

Samples from GSE26975 were used to carry out DE analysis of LDG, SLE neutrophils, and HC neutrophils (FIGS. 57A-57B). This approach identified 657 differentially expressed genes (DEGs) in LDGs compared with SLE neutrophils (173 upregulated, 484 downregulated) (Table 42A), and 224 DEGs compared with HC neutrophils (145 upregulated, 79 downregulated) (Table 42B). No DEGs were noted between SLE neutrophils and HC neutrophils. A total of 132 DEGs were found to be upregulated in LDGs compared with both SLE neutrophils and HC neutrophils.

LDG DEGs included transcripts for granule proteins, cell cycle regulation, chromatin remodeling, cell adhesion, and cytoskeletal regulation. Upregulated genes also included many genes specific to platelets, and downregulated genes included many transcripts for TCR and BCR complexes, suggesting some contamination during neutrophil isolation.

TABLE 42A
LDGs differentially express genes relative to HC neutrophils and
SLE neutrophils (LIMMA DE results for LDGs vs. SLE neutrophils)

| | | | | | |
|---|---|---|---|---|---|
| CD3D | CTSW | LOC101928419 | ECHDC2 | EEF1A1 | TMEM116 |
| ITK | ACRBP | CMTM5 | S1PR5 | TTC27 | INTS2 |
| TRAC | ZC3H12D | FGFBP2 | EXOSC7 | CLEC2D | HAUS5 |
| NA | FAXDC2 | SLAMF1 | IL24 | MAGEE1 | AP3M2 |
| CD2 | TBX21 | HBB | RPS18 | RPL35 | LOC389906 |
| IL2RB | ZNF783 | MMP8 | LINC00342 | LRPPRC | TRMT11 |
| CD8A | CLEC1B | FNBP1L | PHLDB2 | MS4A3 | CAMP |
| GZMK | CD5 | ARHGAP18 | GAS2L1 | NRGN | ZNF23 |
| MYBL1 | TNFSF4 | ST3GAL5 | GLTSCR2 | MRPL9 | SEPT1 |
| SAMD3 | SPX | AUTS2 | LOC440311 | WDR54 | PCID2 |
| TRBC1 | ITGA9 | ARID5B | SH3BGRL2 | CDC14B | FAAP24 |
| LCK | C1orf198 | PIK3IP1 | ANKH | TARP | NMT2 |
| RORA | PBX1 | THEM4 | AK1 | MAP3K7CL | CEACAM8 |
| THEMIS | ATP5E | AXIN2 | GP6 | RPS17 | TOMM5 |
| CDC25B | GGTA1P | DPP4 | TBXA2R | TSPAN33 | STMN3 |
| IL7R | SLC16A7 | MYL9 | PHF10 | CEACAM6 | RPS6 |
| CTLA4 | SDPR | EGF | PASK | LDHB | TPM1 |
| MCUB | BCL2 | AKR1B1 | GP1BA | VIL1 | RPL18 |
| GZMA | GNAZ | CTTN | LCN2 | DDHD2 | LAT |
| RHOBTB1 | PROS1 | CLIP4 | ATP6V0E2 | ZNF121 | DDX47 |
| CD3E | ASAP2 | PRKCH | RNF157 | RALGAPA1 | RPL38 |
| AQP3 | LDOC1L | TRBV27 | LRRC8C | DENND2C | CCDC107 |
| YME1L1 | PTK2 | DOCK10 | CEP78 | ZNF101 | ESAM |
| KLRB1 | RCAN3 | PTCH1 | AHNAK | IMP4 | RPL22 |
| CD6 | CLU | MMD | SUCLG2 | MTFP1 | EPB41L2 |
| INPP4B | KLRD1 | FBL | PDE5A | NOP58 | THBS1 |
| KLRF1 | RAB13 | SLC38A1 | SEC14L5 | SIRPG | MRPL24 |
| BCL11B | DOCK9 | PRDX2 | LGR6 | ATP8B4 | TSHZ1 |
| TRAF5 | RRS1 | ITGB5 | TNS1 | TESPA1 | ATIC |
| STON2 | BIN1 | FLT3LG | TMEM204 | GNL3 | POU6F1 |
| LTBP1 | BEND2 | HEG1 | ETS1 | RPL23A | FAM102A |
| ELOVL7 | C2orf88 | CAMK2N1 | RPS12 | MGLL | RNF125 |
| OPTN | CTDSPL | ZC3HAV1L | MDFIC | FTO | LOC105375427 |
| MAL | GUCY1B3 | TRABD2A | TRIAP1 | CXCL5 | CAMK2D |
| PEAR1 | ITGA2B | C1QBP | PCED1B | DLG3 | TTC3 |
| SH2D1A | PYHIN1 | RRP1B | TTC39B | MYC | GIMAP1 |
| PLEKHA1 | PRF1 | PLA2G12A | ATF7IP2 | UBE2Q2 | TPD52 |
| TAL1 | DAB2 | RAB27B | RPS21 | ERG | RETN |
| SKAP1 | PRKCQ | MSN | POP5 | KIAA1671 | VCL |
| TIAM1 | ARHGAP6 | ASCC3 | NR1D2 | SLC35F2 | EHD3 |
| EOMES | TC2N | RPS19 | CD160 | NLRC3 | NUDCD2 |
| HOPX | SPOCK2 | FCRL3 | RPL10A | RPS23 | RPL24 |
| STAT4 | GNG11 | PPA1 | CYP4V2 | PTGS1 | RPS4X |
| NELL2 | LOC643733 | SUN1 | ALOX12 | MOB1B | SLC12A7 |
| CAMK4 | PF4V1 | OLFM4 | PIK3C2B | LY9 | RSAD1 |
| TMEM40 | NEXN | XCL1 | GPR18 | GTF3A | HINT1 |
| TGFBR3 | KLF12 | DNM3 | MYZAP | GPRASP1 | MUTYH |
| GNLY | F13A1 | HAPLN3 | IRS1 | P2RX7 | CD7 |
| PRKCQ-AS1 | CCR7 | CMC1 | CD27 | SLC18B1 | RPL4 |
| CD247 | GPRIN3 | PF4 | IPO5 | MPL | ZNF639 |
| ITGB3 | MAP4K1 | CD24 | GIMAP6 | IFFO2 | MYCT1 |
| TRDC | LBH | PVT1 | CRISP3 | LYAR | DDX11L2 |
| MYLK | CD8B | PDGFA | DTD2 | MPO | P2RY12 |
| LEF1 | FYN | PGRMC1 | PPBP | PLXND1 | DDB2 |
| TCF7 | IKZF3 | MYB | ITM2A | RPS27 | NREP |
| RASGRP1 | TDRP | FAM35A | RPL3 | APOL3 | NOP14 |
| GIMAP7 | TTC39C | RPL36 | XK | SMIM5 | RPL18AP3 |
| PLCG1 | ZAP70 | TSC22D1 | GSPT1 | CMSS1 | SNRPD2 |
| SPARC | OXNAD1 | POLR3E | PTPRM | HSD17B8 | CLDN5 |
| GZMH | TREML1 | EIF2D | MGC16275 | EPRS | SOD1 |
| GATA3 | SYTL2 | CD3G | OXCT1 | MRPL1 | APRT |
| PRKAR2B | DNMT1 | APBA2 | NOL9 | PET117 | PDGFC |
| TUBB1 | ICOS | SLC4A7 | GOLGA8A | RPL5 | APP |
| MFHAS1 | IL32 | PPP3CC | S100A10 | LTF | TULP4 |
| CD96 | PTPN4 | CDR2 | MFAP3L | SRSF8 | CCDC65 |
| CBLB | CD28 | CRYZ | MEIS1 | PTPRCAP | RPLP0 |

| | | | |
|---|---|---|---|
| RPS5 | GTPBP3 | NOP14-AS1 | FCMR |
| ARHGEF3 | SLAIN1 | TIMM9 | ARL14EP |
| ATP10A | CLECL1 | NOL11 | SERPINE2 |
| DGKH | EVL | DHRS3 | HABP4 |
| LOC100289090 | NSG1 | RPL6 | KLRC3 |
| NSMCE4A | ZNF600 | BCL9L | IGF2BP3 |
| PTGER2 | RDH11 | TYW3 | USP46 |
| LOC647115 | RPL14 | ATP8B2 | RDH13 |
| OSBPL3 | PRSS23 | ABHD14B | GNPNAT1 |
| RPL32 | C14orf169 | LOC105371967 | CHIT1 |
| TRAP1 | FAM84B | ARHGAP21 | SMOX |
| USP36 | ABLIM3 | IL11RA | CCDC25 |
| C15orf54 | TMEM45A | IARS | MAK16 |
| PEBP1 | SSRP1 | VPS50 | TMEM158 |
| RPS7 | LOC101928152 | ZNF84 | XPOT |
| LRRN1 | UBASH3A | GCFC2 | ICE1 |
| TBC1D4 | ATP9A | PRKD3 | TBC1D31 |
| IL27RA | AGK | DNAJA3 | AARS |
| RPL35A | PPRC1 | MATR3 | ELAC2 |
| RPS28 | WDR75 | SLC25A38 | PSMD14 |
| HMGN3 | RPS25 | RPL9 | NUDT15 |
| AKAP7 | ANLN | INHBA | ABCA13 |
| PIK3R1 | AES | NEIL2 | REXO2 |
| MLLT3 | BEX3 | TMEM173 | KIFC2 |
| RPS10 | RPL11 | GYPC | GPR171 |
| RPS27A | CFAP97 | NPIPA1 | MGAT4A |
| TMEM267 | RPL31 | ASF1A | VSIG2 |
| TFPI | MTURN | ZBTB25 | FEZ2 |
| LOC102724587 | RRM2 | STOM | AGPAT4 |
| MTX3 | MRPL50 | UTP4 | RPS3 |
| AZU1 | IDI2 | MLH3 | SMIM3 |
| NCALD | CDK4 | CCDC88C | LRLG1 |
| MYNN | ATG14 | PDP1 | DEFA4 |
| PDZD4 | ALKBH8 | PPAT | MLLT6 |
| TRMT61B | RPL37A | CTSG | SMARCAD1 |
| CCT2 | WDR60 | SUPV3L1 | STK39 |
| RASGRF2 | POLR1C | PPP1R16B | MPHOSPH10 |
| PCCA | TMEM263 | SDAD1 | PPP2R2B |
| SLC41A1 | ADGRG1 | TFF3 | KLAA0101 |
| NUP210 | DMTN | MYBBP1A | SLC39A8 |
| SUMF2 | LINC01278 | EIF3A | AKT3 |
| YWHAH | S1PR1 | MRPL3 | SSB |
| XPO4 | CAPN2 | SPOCD1 | FBXO41 |
| EBAG9 | LYRM4 | RRAS2 | TUSC1 |
| LINC01215 | BNIP3 | ALDH18A1 | KIAA1147 |
| RPL13A | UBA2 | IL10RA | BEX1 |
| RPL8 | TSPAN17 | A2M-AS1 | EEF2 |
| EIF2A | FBXL16 | AMIGO1 | MRPS18B |
| SYTL4 | LOC105370792 | CCR6 | DACH1 |
| CDCA7L | BRMS1L | DENND2D | C12orf57 |
| RRP15 | ZNF558 | SSX2IP | KPNA3 |
| FARSB | NLK | RPL27A | TYSND1 |
| BMS1 | FAM159A | RPL19 | WDR3 |
| CHIC1 | POGLUT1 | ANKRD33B | ANO6 |
| DCLRE1A | MAP4 | TTC7B | BET1 |
| ZNF331 | ZNF831 | SGO2 | DYRK2 |
| NDFIP1 | NOC3L | PTMS | AEN |
| UGCG | NCR3LG1 | FRMD3 | NCAM1 |
| RPLP2 | ELANE | NCL | CACNA1I |
| ACTR5 | RPS15A | URI1 | CEBPZ |
| IL23A | SYNCRIP | EPB41L4A-AS1 | GATA2 |
| ADA | RPS16 | NDUFA12 | GNGT2 |
| SCD | ABCC3 | TARSL2 | TMEM42 |
| FKBP11 | SSBP4 | PFKP | |
| DGKE | LOC100131689 | P2RY1 | |
| RPS29 | RPS28P6 | HNRNPDL | |

TABLE 42B
LDGs differentially express genes relative to HC neutrophils and SLE neutrophils (LIMMA DE results for LDGs vs. HC neutrophils)

| | | | | | | | | | |
|---|---|---|---|---|---|---|---|---|---|
| TRAC | RORA | PBX1 | TCF7 | RAB13 | RNF217 | KLRF1 | ZC3H12D | EHD3 | OPTN |
| GZMK | PTK2 | GGTA1P | CD247 | FNBP1L | TMEM158 | LTF | FGFBP2 | CTLA4 | ABCC3 |
| TUBB1 | ITK | MAP3K7CL | LOC101928419 | BEX3 | TNS1 | UGCG | TTC7B | CLDN5 | AUTS2 |
| CD3D | ITGB3 | NEXN | TRBV27 | MYCT1 | CD6 | ZC3HAV1L | TRDC | P2RY12 | ABLIM3 |
| GNG11 | YME1L1 | ITGA2B | ACRBP | THEMIS | PTGS1 | TBX21 | HOPX | DCBLD2 | MPO |
| PRKAR2B | CLEC1B | HBB | ITGB5 | DNM3 | SAMD3 | IKZF3 | MPL | CD36 | CD3G |
| RHOBTB1 | MCUB | PRKCQ-AS1 | PROS1 | IRS1 | GP1BA | STAT4 | CD28 | ARHGAP18 | SMOX |
| ATP5E | PF4 | GUCY1B3 | MMD | TGFBR3 | SEC14L5 | RDH11 | LOC643733 | CEACAM6 | CLIC4 |
| CD8A | CLU | ARHGAP6 | SH3BGRL2 | PDGFC | CD2 | PLAC8 | PLCG1 | GATA3 | SCD |
| LTBP1 | DAB2 | NRGN | PGRMC1 | EOMES | GPX1 | CEACAM8 | XCL1 | AZU1 | BCL2 |
| NA | SDPR | TNFSF4 | MYL9 | PDE5A | APP | KLF12 | ATP8B4 | CD8B | FYN |
| ELOVL7 | FAXDC2 | TREML1 | EGF | PRF1 | OLFM4 | CDC14B | ERG | SH2D1A | ABCA13 |
| KLRB1 | CTDSPL | PLEKHA1 | MMP8 | CXCL5 | MGLL | VCL | MITF | TRAF5 | JAM3 |
| AQP3 | LEF1 | GZMA | IL2RB | PF4V1 | NELL2 | AXIN2 | MS4A3 | GP6 | MRPS26 |
| STON2 | BEND2 | MYB | CTTN | RN7SL587P | GZMH | THBS1 | H1F0 | ANO6 | DPP4 |
| TAL1 | XK | RASGRP1 | LCN2 | TSC22D1 | SPOCK2 | INPP4B | DYNLL1 | MTURN | DMTN |
| TRBC1 | C1orf198 | DENND2C | PLA2G12A | MFAP3L | TSPAN33 | C15orf54 | BEX1 | MLH3 | NUSAP1 |
| PEAR1 | SPX | ASAP2 | PPBP | CRISP3 | MYZAP | RCAN3 | RETN | GNLY | |
| TMEM40 | C2orf88 | PDGFA | LBH | SMIM5 | PRKCH | CCR7 | ETS1 | SYTL4 | |
| IL7R | ITGA9 | GAS2L1 | TBXA2R | CDC25B | IL32 | DDX11L2 | ESAM | PYHIN1 | |
| GNAZ | MYLK | CDCA7L | MOB1B | RAB27B | PHLDB2 | CTSW | FRMD3 | TFPI | |
| MYBL1 | BCL11B | CMTM5 | GIMAP7 | CD24 | CD3E | LCK | CLCN3 | PASK | |
| SPARC | F13A1 | TDRP | MSN | MEIS1 | ALOX12 | MAL | CAMK4 | GOLGA8A | |

### Example 20: Delineation of LDG-Specific Modules of Genes from WGCNA

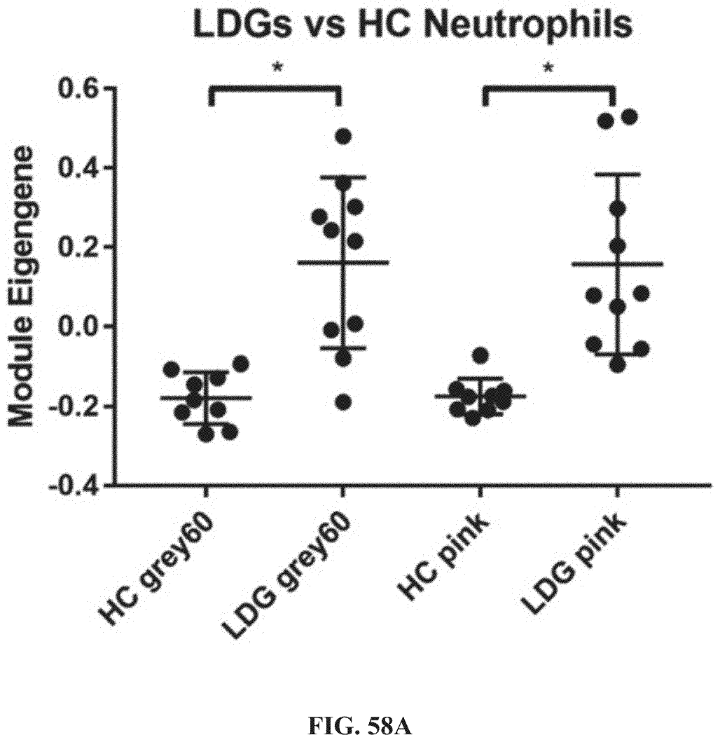

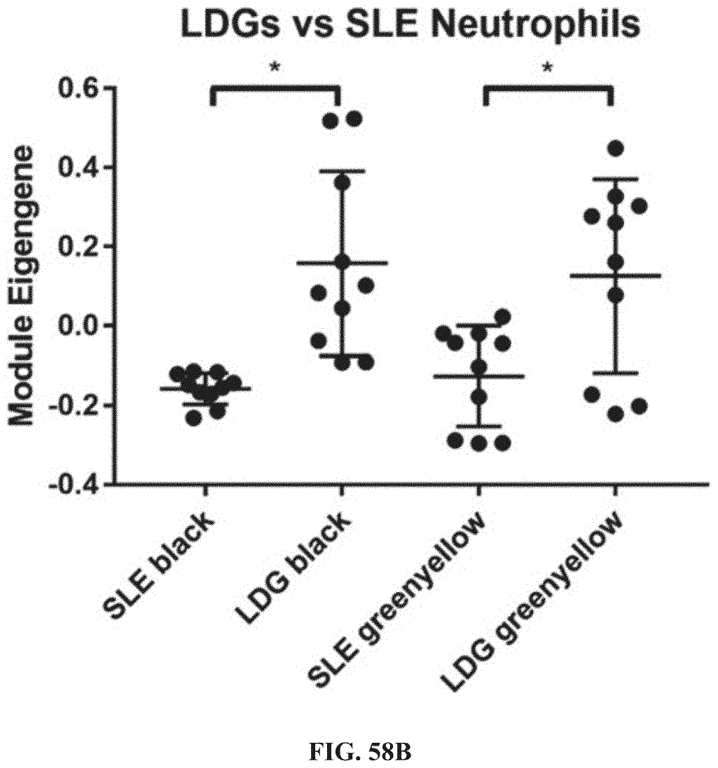

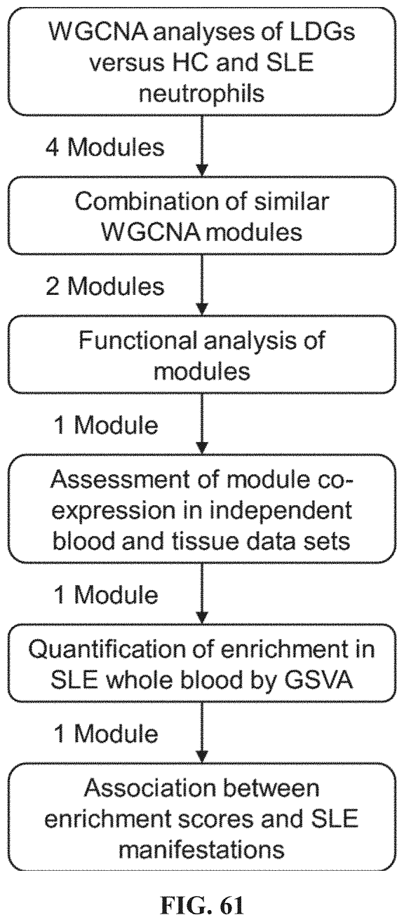

Because of the potential effects of cellular contamination on the DEG analysis, a computational approach was developed to identify highly discriminatory genes characteristic of LDGs that could be used to facilitate downstream analyses in blood and tissues. An overview of this process can be found in FIG. 61. Identifying groups of coexpressed genes may minimize the effects of potential contamination and enable better characterization of LDGs by separating LDG-specific genes from lymphocyte- and platelet-specific groups of genes. Samples from GSE26975 were used to carry out unsupervised WGCNA of LDGs, SLE neutrophils, and HC neutrophils to identify modules of potentially informative genes based on coexpression rather than known experimental design (FIGS. 58A-58B). This approach initially generated 56 modules of genes. Six of these had ME values that were significantly increased or decreased in LDG samples by Welch's t test (p<0.05) (Tables 42A-42C). One module (midnightblue) was removed from consideration upon inspection because its ME values did not differ from SLE neutrophils in a majority of samples. Another module (mediumpurple3) was removed because it contained transcripts from B and T cells, an indication that the WGCNA approach could filter out contamination.

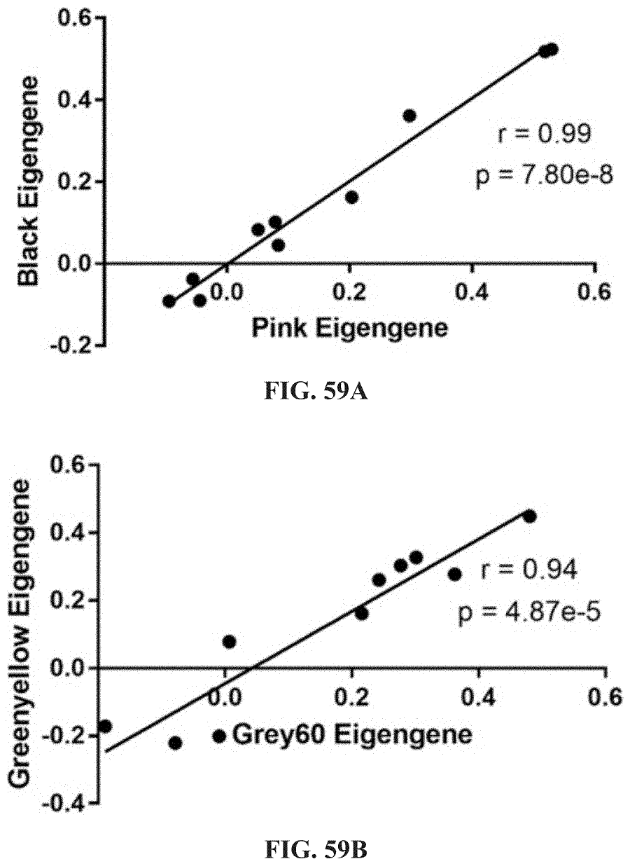

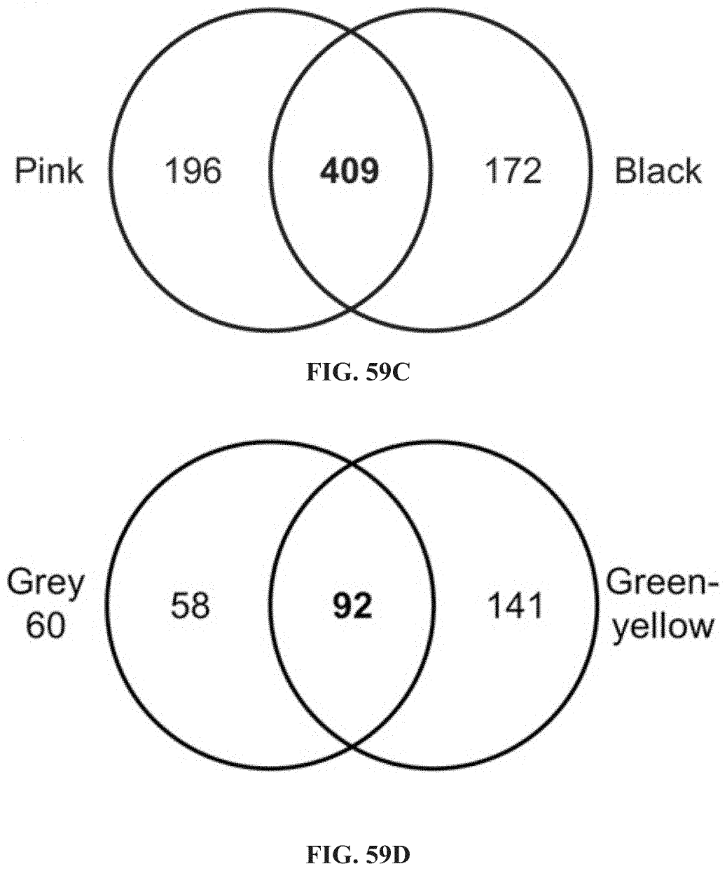

The remaining four modules (pink, black, grey60, and greenyellow (Tables 42A-42C) were compared based on their ME values in the LDG expression data and the genes that comprised them. The pink and black modules had strongly correlated MEs (r=0.99, p=7.80×10⁻⁸) and shared 409 genes (FIGS. 59A and 59C). Similarly, the grey60 and greenyellow modules had correlated MEs (r=0.94, p=4.87×10⁻⁵) and shared 92 genes (FIGS. 59B and 59D). All other ME correlations were not significant (NS) (all p>0.6). Modules were then consolidated with the goal of acquiring a gene signature that could robustly set LDGs apart from both HC neutrophils and SLE neutrophils. The pink and black modules were combined to form module A (Table 43A), and the greenyellow and grey60 modules were combined to form module B (Table 43B). These modules were then subjected to functional analysis to identify a specific, robust LDG gene signature.

TABLE 43A
Genes in LDG Module A (by gene ontology designation)

| | | Positive | | | | |
|---|---|---|---|---|---|---|
| | | Regulation | | | | |
| | | of Protein | | | | |
| Leukocyte | Platelet | Kinase | | | | |
| Migration | Degranulation | Activity | Other | | | |
| ANO6 | PF4 | SDC4 | ABCC3 | DPPA4 | MPL | SLC35D3 |
| CXCL5 | PPBP | ADCY3 | ABHD15 | DPYSL2 | MSANTD3 | SLC44A1 |
| ESAM | ITGB3 | ADRA2A | ABI2 | DST | MTHFD2L | SLC8A3 |
| GP6 | CLU | AFAP1L2 | ABLIM3 | EGLN3 | MTMR2 | SMAD1 |
| GRB14 | PDGFA | CCDC88A | ACER3 | EHD3 | MTURN | SMIM24 |
| HMGB1 | THBS1 | F2R | ACRBP | ELOVL7 | MYB | SMIM5 |
| IGKC | ABCC4 | GADD45A | ACSBG1 | ENKUR | MYCT1 | SMOX |
| IGLC1 | APP | MMD | ACVR1 | EPB41L3 | MYL9 | SNAPC3 |
| ITGA9 | CD36 | NCAPG2 | AFAP1 | ERG | MYLK | SNPH |
| ITGB1 | DMTN | PDE5A | AFF3 | ERV3-1 | MYNN | SOX4 |
| JAM3 | EGF | PDGFC | AGBL5 | FAM20B | NAP1L1 | SPOCD1 |
| MSN | ENDOD1 | PRKAR2B | AGPAT5 | FAM212B-AS1 | NAT8B | SPSB1 |
| PF4V1 | F13A1 | PTK2 | AIG1 | FAM65C | NCK1-AS1 | SPX |
| THRB | ITGA2B | SNCA | AKIP1 | FAM69B | NCKAP1 | SSX2IP |
| | MMRN1 | STRADB | ALDH1A1 | FAM81B | ND4 | ST3GAL3 |
| | PROS1 | TAL1 | ALOX12 | FAXDC2 | NENF | STMN1 |
| | RAB27B | TCL1A | ANKRD28 | FHL1 | NEXN | STON2 |
| | SPARC | TNIK | AP1S2 | FHL2 | NIPA1 | SYNM |
| | SYTL4 | | AQP10 | FKBP1B | NLK | TARBP1 |
| | VCL | | AR | FNBP1L | NORAD | TBXA2R |
| | VWF | | ARHGAP18 | FRMD3 | NPRL3 | TCEAL8 |
| | | | ARHGAP21 | FSTL1 | NREP | TCF4 |
| | | | ARHGAP32 | GAS2L1 | NRGN | TDRP |
| | | | ARHGAP6 | GGTA1P | NT5M | TEX2 |
| | | | ARHGEF12 | GLCE | NUTM2A-AS1 | TFB1M |
| | | | ARMCX3 | GMPR | OPN3 | TFPI |
| | | | ASAP2 | GNA12 | P2RY12 | TGFB1I1 |
| | | | ATP5E | GNAZ | PANX1 | TGFBI |
| | | | ATP5S | GNG11 | PARD3 | TLK1 |
| | | | ATP9A | GNG8 | PARVB | TLR7 |
| | | | AVPR1A | GP1BA | PAWR | TMCC2 |
| | | | B4GALT6 | GP5 | PBX1 | TMEM158 |
| | | | BACE1 | GPX1 | PCYT1B | TMEM40 |
| | | | BCL11A | GRAP2 | PDE2A | TMEM45A |
| | | | BCL2L1 | GSTP1 | PDE3A | TMEM64 |
| | | | BCL2L2 | GUCY1A3 | PDLIM1 | TNFSF4 |
| | | | BEND2 | GUCY1B3 | PDZD2 | TNS1 |
| | | | BET1 | H1F0 | PDZK1IP1 | TNS3 |
| | | | BEX3 | H2AFJ | PEAR1 | TPM1 |
| | | | BICD1 | HEMGN | PGRMC1 | TPSAB1 |
| | | | BLNK | HEXIM2 | PITPNM2 | TPSB2 |
| | | | BMP6 | HGD | PKHD1L1 | TPST2 |
| | | | BMP8B | HIST1H2AE | PKIG | TPTEP1 |
| | | | C12orf75 | HIST1H2BJ | PLA2G12A | TRBV27 |
| | | | C12orf76 | HIST1H2BO | PLEKHA8P1 | TREML1 |
| | | | C15orf52 | HIST1H4I | PLOD2 | TRIM10 |
| | | | C15orf54 | HMGN1 | PNMA1 | TRIM13 |
| | | | C19orf33 | HRASLS | PPM1L | TRIM58 |
| | | | C1orf198 | IGF2BP3 | PRDX6 | TSC22D1 |
| | | | C2orf88 | IRS1 | PRG2 | TSPAN18 |
| | | | C7orf73 | ITGB5 | PROSER2 | TSPAN33 |
| | | | CA13 | KALRN | PRTFDC1 | TSPAN9 |
| | | | CA2 | KCND3 | PRUNE1 | TTC7B |
| | | | CALD1 | KIF2A | PSD3 | TUBB |
| | | | CAMTA1 | KLHL5 | PSPH | TUBB1 |
| | | | CANX | LAPTM4B | PTCRA | TWSG1 |
| | | | CASP6 | LGALSL | PTGIR | UBE2E2 |
| | | | CD151 | LINC00853 | PTGS1 | UBE2O |
| | | | CD226 | LINC00938 | PTPN18 | UBL4A |
| | | | CDC14B | LIPH | PTPRS | UGCG |
| | | | CDIP1 | LMNA | PXDC1 | USP12 |
| | | | CDK2AP1 | LOC101928419 | PYGB | USP31 |
| | | | CDK6 | LOC105371967 | RAB13 | UXS1 |
| | | | CDKL1 | LOC105377276 | RAB30 | VEPH1 |
| | | | CDYL | LOC283194 | RAP1B | VIL1 |
| | | | CHD9 | LPAR5 | RAP2B | VSIG2 |
| | | | CLCN3 | LRBA | RBPMS2 | VWA5A |
| | | | CLDN5 | LTBP1 | RCC2 | WASF1 |
| | | | CLEC1B | LYPLAL1 | RDH11 | WASF3 |
| | | | CLIC4 | LZTS2 | RGS10 | WDR11-AS1 |
| | | | CMTM5 | M1AP | RHBDD1 | WHAMMP2 |
| | | | CNRIP1 | MAGI2-AS3 | RHOBTB1 | WRB |
| | | | CNST | MAGOHB | RNF11 | WWC1 |
| | | | COMT | MAP1A | RNF217 | XK |
| | | | CPED1 | MAP1B | RSU1 | XPNPEP1 |
| | | | CPNE5 | MAP3K7CL | SAV1 | YIF1B |
| | | | CRAT | MAST4 | SCFD2 | YWHAE |
| | | | CRLS1 | MAX | SCN9A | YWHAH |
| | | | CTC-338M12.4 | MBTD1 | SDPR | ZBTB16 |
| | | | CTDSPL | MCM6 | SEC14L5 | ZC3HAV1L |
| | | | CTTN | MCUR1 | SEPT11 | ZNF175 |
| | | | DAAM1 | MEIS1 | SERPINE2 | ZNF271P |
| | | | DAB2 | MEST | SH3BGRL2 | ZNF367 |
| | | | DCLRE1A | MFAP3L | SH3TC2 | ZNF431 |
| | | | DDX11L2 | MGLL | SHTN1 | ZNF521 |
| | | | DENND2C | MINPP1 | SIAE | ZNF529-AS1 |
| | | | DIMT1 | MITF | SLA2 | ZNF542P |
| | | | DNAJC6 | MLH3 | SLC25A43 | ZNF677 |
| | | | DNM3 | MOB1B | SLC35D2 | ZNF718 |

TABLE 43B
Genes in LDG Module B (by gene ontology
sub-module designation)

| Neutrophil | | | | |
|---|---|---|---|---|
| Degranulation | | Cell Cycle | Other | |
| ABCA13 | MPO | ANLN | AGPS | MED7 |
| ARG1 | MS4A3 | BIRC5 | ANXA4 | NFYC |
| ATP8B4 | OLFM4 | BUB1B | ATP23 | NUCB2 |
| AZU1 | OLR1 | CCNA1 | BCL2L15 | PCOLCE2 |
| CAMP | RNASE3 | CDK1 | BEX1 | PDLIM5 |
| CEACAM6 | SERPINB10 | CDKN2B | CD24 | PLEKHA3 |
| CEACAM8 | SLC2A5 | DHFR | CTBP2 | PPFIA4 |
| CHIT1 | STOM | GFI1 | CTC1 | RPE |
| CLEC12A | TCN1 | INHBA | DCBLD2 | SCD |
| CLEC5A | | IQGAP3 | ECRP | SENP1 |
| CPNE3 | | KIAA0101 | ERG | SLC28A3 |
| CRISP3 | | KIF11 | FBXO9 | SMIM8 |
| CTSG | | KIF14 | GALNT10 | TACSTD2 |
| CYBB | | KNL1 | GCLM | TCTEX1D1 |
| DEFA4 | | MIS18BP1 | GLOD5 | THBS4 |
| ELANE | | NCAPG | GVINP1 | TMEM234 |
| HP | | RGCC | HMGB2 | TMEM50B |
| LCN2 | | RRM2 | HMGN2 | TMLHE |
| LTF | | SKA2 | KBTBD6 | TRMT5 |
| MGST1 | | TOP2A | LINC00323 | ZNF788 |
| MMP8 | | TYMS | LMO4 | |

TABLE 43C
LDG vs. HC Grey 60

| | | | | | | | | | |
|---|---|---|---|---|---|---|---|---|---|
| ABCA13 | CAMP | CPNE3 | ECRP | HGF | LINC00323 | NUCB2 | POLR2J4 | SLC28A3 | TMLHE |
| AGPS | CCNA1 | CPT1A | ELANE | HIPK2 | LINC00622 | NUSAP1 | PPFIA4 | SLC2A5 | TOP2A |
| ANLN | CD24 | CRISP2 | EMB | HLA-DOB | LINC00674 | OLFM4 | PRKAG2 | SMIM8 | TRAF3IP2 |
| ANXA4 | CDH26 | CRISP3 | ERG | HMBOX1 | LMO4 | OLR1 | RBBP6 | SOCS4 | TRMT5 |
| ARG1 | CDK1 | CSTF3 | EXOSC3 | HMGB2 | LOC101927451 | PCMT1 | RGCC | SPTBN1 | TYMS |
| ARPP19 | CDKN2B | CTBP2 | FAM46A | HMGN2 | LTF | PCOLCE2 | RLF1 | STOM | UGCG |
| ATP23 | CEACAM6 | CTC1 | FBXO9 | HP | MED7 | PDGFRA | RNASE3 | SYNE1 | YIPF5 |
| ATP8B4 | CEACAM8 | CTSG | FUT4 | INHBA | MGST1 | PDLIM5 | RPE | TACSTD2 | ZC3H12C |
| AZU1 | CHIT1 | CYBB | GAB1 | IQGAP3 | MIS18BP1 | PECR | RRM2 | TAF8 | ZNF418 |
| BCL2L15 | CHPT1 | DCBLD2 | GALNT10 | KBTBD6 | MMP8 | PEX13 | SCD | TCN1 | ZNF586 |
| BEX1 | CLEC12A | DDX49 | GCLM | KIAA0101 | MPO | PGGT1B | SCUBE3 | TCTEX1D1 | ZNF615 |
| BIRC5 | CLEC12B | DEFA4 | GFI1 | KLF11 | MS4A3 | PIWIL4 | SDCBP2-AS1 | THBS4 | ZNF788 |
| BUB1B | CLEC5A | DENR | GFPT1 | KLF14 | NBPF1 | PKNOX1 | SENP1 | TIA1 | ZNRF1 |
| C20orf27 | CLMN | DHFR | GLOD5 | KNL1 | NCAPG | PLAC8 | SERPINB10 | TMEM234 | ZSCAN30 |
| CACNB2 | CNOT7 | DNAJC13 | GVINP1 | LCN2 | NFYC | PLEKHA3 | SKA2 | TMEM50B | ZWINT |

### Example 21: Functional Analysis of LDG Gene Modules

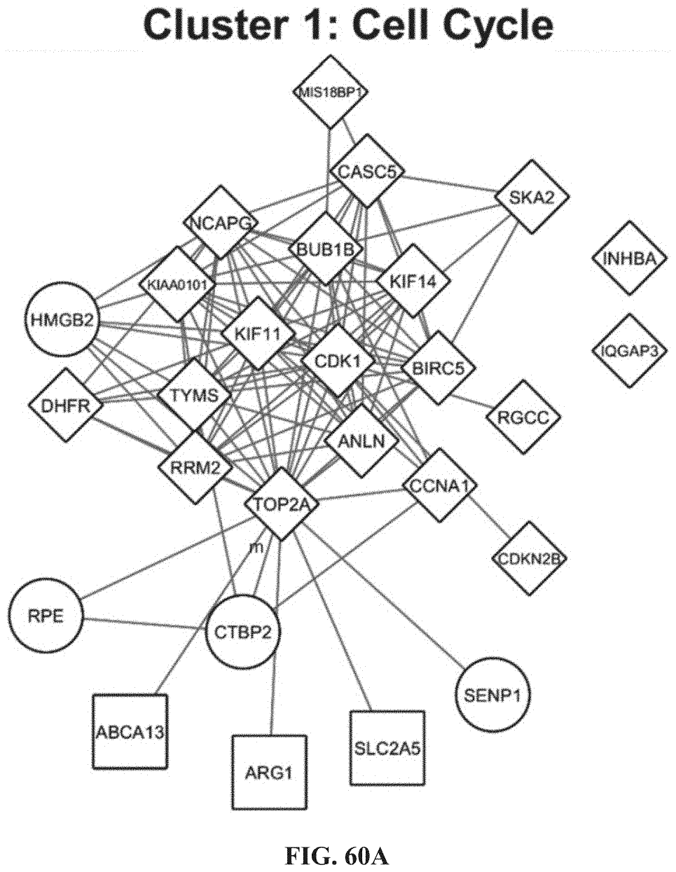

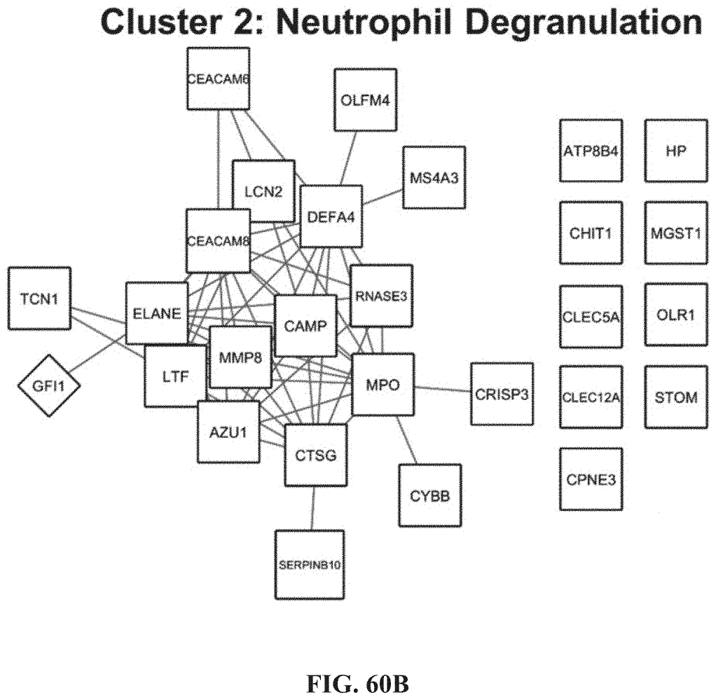

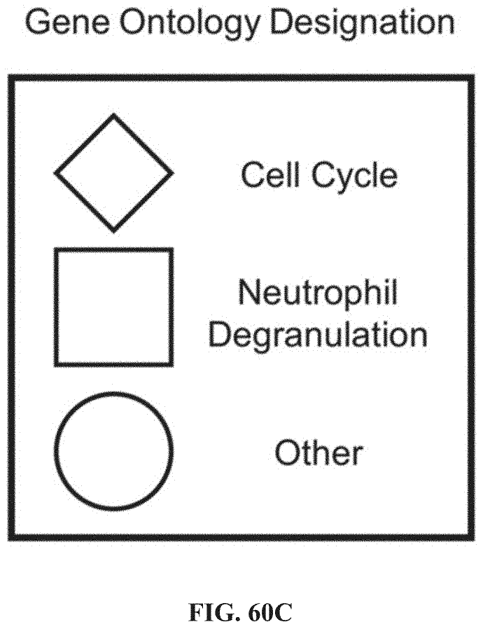

Protein—protein interaction networks were generated for each consolidated LDG module in STRING and sorted to form clusters in Cytoscape using MCODE. Functional analysis of clustered genes showed that module B contained a strongly interconnected cluster of neutrophil granule genes and another cluster of genes associated with DNA synthesis and cell cycle regulation (FIGS. 60A-60C). Overall, 30 of 92 genes in module B had the neutrophil degranulation Gene Ontology (GO) designation, including AZU1, CAMP (LL-37), CTSG, DEFA4, ELANE (ELA2), LCN2 (NGAL), LTF, MMP8, MPO, and RNASE3. Additionally, 21 of 92 genes in module B had the cell cycle GO designation. Of the 41 remaining genes, there were several genes encoding typical LDG surface proteins, including CD66b, and also genes involved in transcriptional regulation, but no overall function for the remaining genes could be determined. Because circulating neutrophils do not express granulopoietic genes and because SLE neutrophils do not differentially express any genes relative to HC neutrophils, the presence of this module of genes in the blood or tissue of SLE patients was likely to be attributable to LDGs and not merely neutrophil activity.

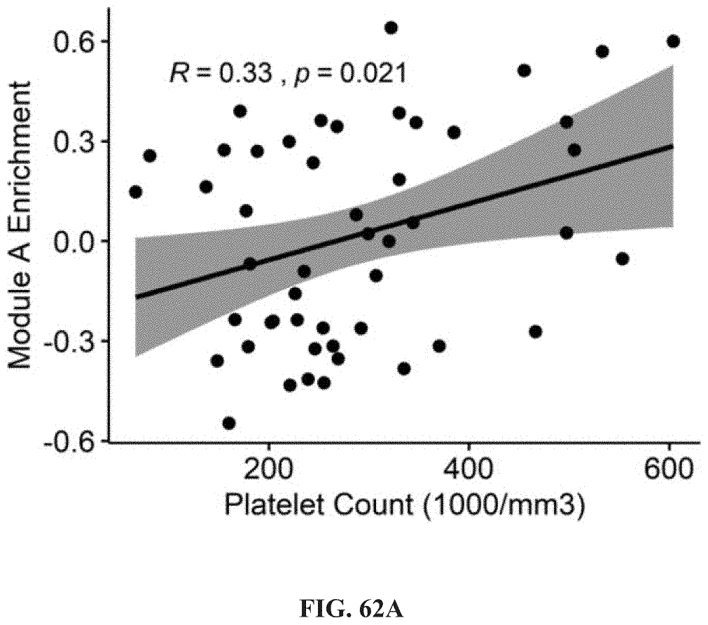

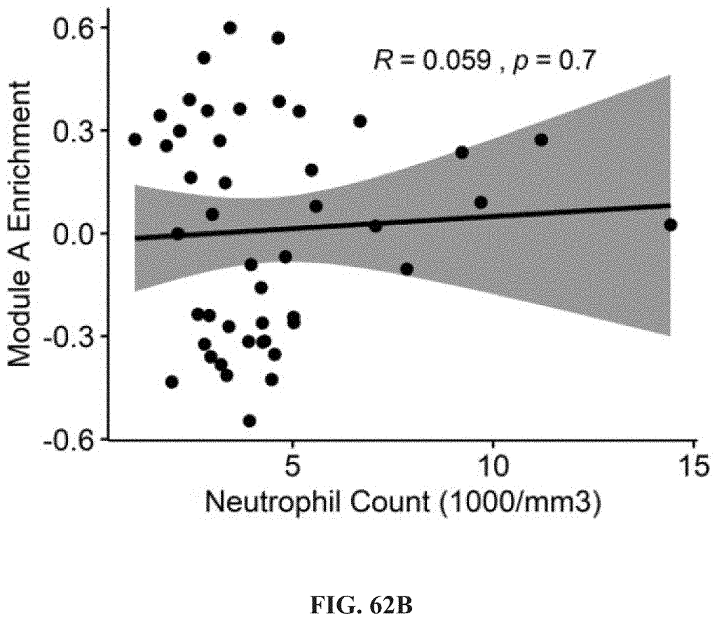

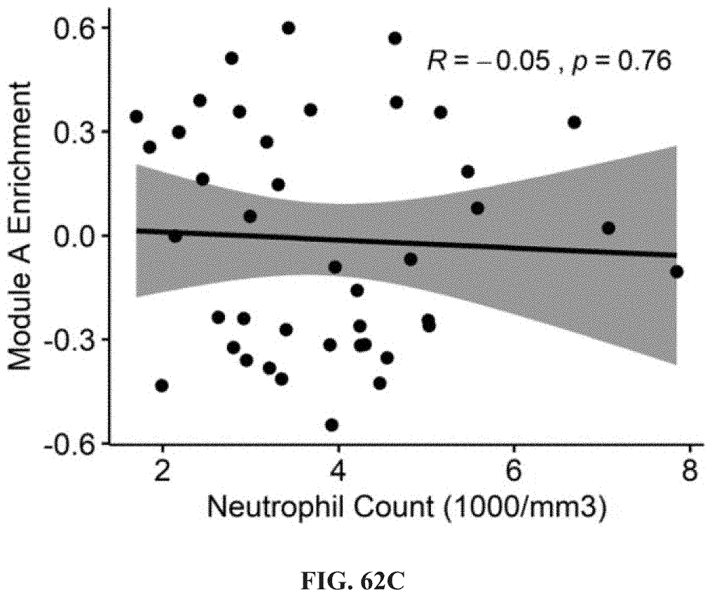

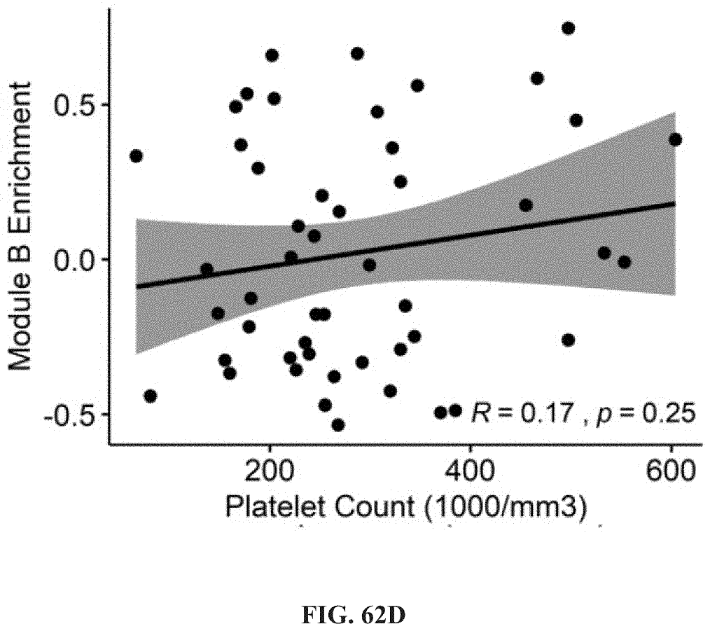

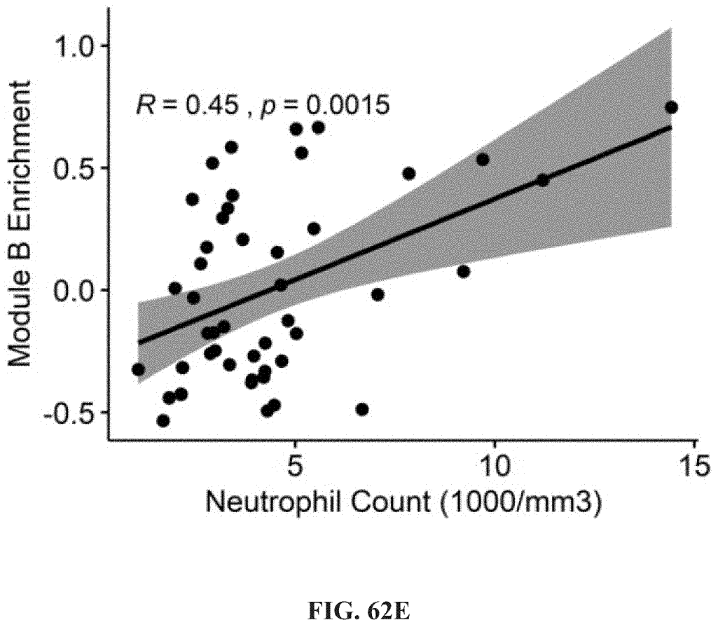

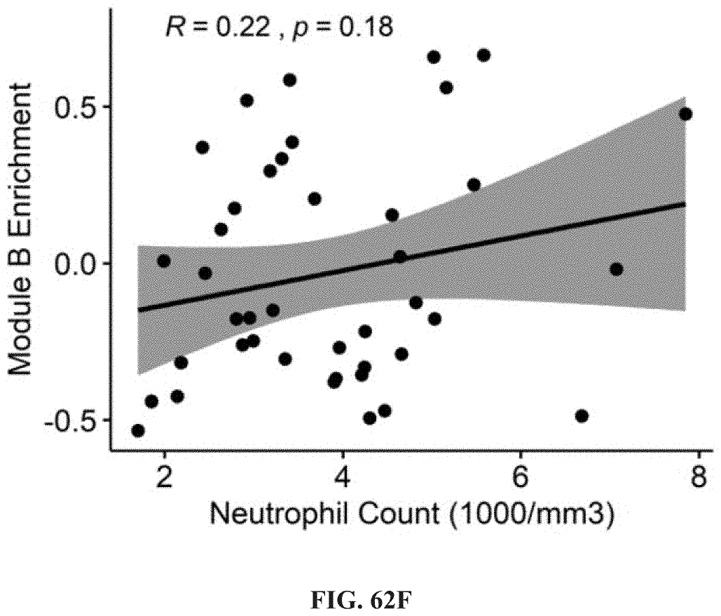

To confirm that module B was not related to neutrophil activation, module B genes were compared with 809 genes differentially expressed by activated human neutrophils in experimental endotoxemia at three time points. Of the 809 activated neutrophil genes (Table 44A), only 18 genes (2.2%) (Table 44B) were found among the 92 genes in module B (Table 43B), of which 13 (Table 44C) are also among the 30 module B genes implicated in neutrophil degranulation. Although module B bears some similarity to this activated neutrophil signature (18 of 92 genes), it retains a unique array of granule proteins (AZU1, CAMP, CHIT1, CTSG, DEFA4, ELANE, LTF, MPO, RNASE3), cell cycle proteins, and surface markers (CD24, CD66b, CD66c, CLEC12A, MS4A3) that set it apart. Furthermore, an analysis of LDG module B GSVA enrichment scores in GSE49454 WB showed only a minimal association between LDG enrichment and neutrophil count (r=0.45, p=0.0015), which lost significance when patients with extremely high or low neutrophil counts were excluded (r=0.22, p=0.18) (FIGS. 62A-62F). These results implied that module B genes did not reflect either neutrophilia or neutrophil activation, and therefore module B was chosen to query blood and tissue gene expression data for the presence of LDGs.

Module A contained many genes associated with intracellular signaling as well as genes specific for platelets that may have been coisolated with LDGs during separation (Table 43A). In GSE49454 WB, module A enrichment scores showed a correlation with platelet counts (r=0.33, p=0.02) and no correlation with neutrophil counts (p>0.6) (FIGS. 62A-62F). Although this module of genes could be informative for studying the biology of LDGs, it was not sufficiently specific to query blood and tissue expression data for the presence of LDGs.

TABLE 44A
Activated neutrophil genes

| | | | | | | | | | |
|---|---|---|---|---|---|---|---|---|---|
| ABCA5 | C16orf7 | CYBA | FKBP5 | HP | LRRFIP2 | OLAH | RAB7L1 | SLC36A4 | TNIP1 |
| ABCF1 | C16orf87 | CYBB | FLJ10213 | HPR | LRRN1 | OLFM4 | RABGEF1 | SLC37A3 | TNPO3 |
| ABL2 | C19orf59 | CYC1 | FLJ20323 | HPS5 | LSDP5 | OLR1 | RALB | SLC39A8 | TOMM40L |
| ABP1 | C19orf61 | CYFIP2 | FLJ22662 | HRB | LSM6 | OPLAH | RALGDS | SLC43A3 | TOR1B |
| ACAA1 | C1GALT1 | DACH1 | FLJ31222 | HS3ST3B1 | LYPD3 | OR2M7 | RANBP2 | SLC44A1 | TP53BP2 |
| ACAT2 | C1orf162 | DAPP1 | FLJ34047 | HSD17B12 | M6PRBP1 | ORC2L | RASA2 | SLC7A5 | TP53I11 |
| ACN9 | C20orf3 | DBNL | FLJ36031 | HSPA1B | MAD2L2 | ORM1 | RBM11 | SLC9A8 | TP53I3 |
| ACO1 | C20orf74 | DCTN6 | FLJ43692 | HSPA1L | MAFG | ORM2 | RCHY1 | SLCO4C1 | TPPP |
| ACOT9 | C21orf91 | DCUN1D3 | FLOT1 | HTRA3 | MAP1LC3A | OSBPL6 | REC8 | SLK | TRAF1 |
| ACP6 | C2orf56 | DDAH2 | FLOT2 | ICAM1 | MAP2K1 | OSBPL9 | REL | SMPD2 | TRAF3 |
| ACPL2 | C3 | DDIT4 | FLYWCH2 | ICAM4 | MAP2K6 | OSCAR | RELB | SMPDL3A | TRAF3IP3 |
| ACSL3 | C3AR1 | DDX21 | FMNL3 | ID3 | MAP4K4 | OSM | RFX3 | SMU1 | TRIB1 |
| ACVR1B | C3orf19 | DDX23 | FNDC3A | IDI1 | MAPK14 | OSTalpha | RFX5 | SNAPIN | TRIP10 |
| ADAM15 | C3orf21 | DDX58 | FNTA | IER3 | MARCH3 | OXSR1 | RHOH | SNORD49A | TRMT6 |
| ADAM9 | C3orf23 | DEGS1 | FOLR3 | IER5 | MARCKS | P4HA1 | RHOU | SNX20 | TRPM2 |
| ADD3 | C4orf16 | DENND1B | FOSL1 | IFI16 | MBOAT1 | PADI4 | RIF1 | SNX3 | TRPM6 |
| ADM | C5orf22 | DENND2D | FPGT | IFI35 | MCM8 | PAM | RIPK2 | SOCS3 | TRPS1 |
| ADORA2A | C5orf29 | DHCR7 | FSCN1 | IFI44 | MCTP1 | PANK3 | RMND5B | SOD2 | TRPV2 |
| AGL | C5orf32 | DHRS13 | FUT4 | IFI44L | MCTP2 | PARP10 | RNASEL | SORT1 | TSNAX |
| AGPAT6 | C6orf150 | DHX36 | FUT7 | IFIH1 | MED20 | PCMT1 | RNF10 | SPATA13 | TSPO |
| AGPAT9 | C6orf167 | DIAPH2 | G0S2 | IFT20 | MEF2A | PDE4B | RNF144B | SPATC1 | TTC38 |
| AGXT2L2 | C6orf211 | DLEC1 | GADD45A | IKBKE | MEI1 | PDHA1 | ROPN1L | SPECC1L | TTL |
| AIM2 | C9orf123 | DMXL1 | GADD45B | IL10RA | METTL6 | PDSS1 | RP5-1077B9.4 | SPINT2 | TTLL12 |
| ALAS1 | C9orf46 | DNAJA1 | GALC | IL18 | METTL9 | PDXK | RPH3A | SPP1 | TTN |
| ALDH1A1 | CACHD1 | DNAJC25 | GALNS | IL18R1 | MFSD2 | PECR | RPH3AL | SPPL2A | TTPAL |
| ALDH2 | CAMK1G | DNTTIP2 | GALNT1 | IL18RAP | MFSD9 | PEF1 | RPL15 | SPRED2 | TWSG1 |
| ANKRD22 | CAMKK2 | DOT1L | GALNT2 | IL1A | MGC72080 | PFKFB2 | RPL35A | SRI | TXNDC3 |
| ANKRD34B | CAPG | DPYD | GALNT4 | IL1B | MLH1 | PFKFB3 | RPL4 | SRP54 | UBA6 |
| ANKRD55 | CAPN3 | DRAM | GALNT7 | IL1R2 | MLKL | PFTK1 | RPS6KA2 | SRPK1 | UBE2E1 |
| ANKRD6 | CARD6 | DSE | GBA | IL1RN | MLLT6 | PGLYRP1 | RRAGB | ST3GAL2 | UBE2F |
| ANO10 | CARS2 | DUSP13 | GBE1 | IL4R | MMP8 | PGM1 | RRAGD | ST6GALNAC3 | UBE2H |
| ANP32E | CASP1 | DUSP5 | GBGT1 | IL6ST | MMP9 | PHACTR2 | RSBN1 | STAU2 | UBQLN2 |
| ANXA3 | CAST | DYNLT3 | GBP1 | IMPA1 | MOSC1 | PHCA | RSU1 | STOM | UBR1 |
| ANXA4 | CCDC134 | DYSF | GCH1 | INPP1 | MOSPD3 | PHTF1 | RTN2 | STX11 | UEVLD |
| ANXA7 | CCL20 | ECHDC1 | GCLM | IRAK2 | MPP7 | PIK3AP1 | RY1 | STYXL1 | UGCG |
| APOA2 | CCL4 | ECHDC3 | GFOD2 | IRAK3 | MPZL2 | PIM2 | S100A12 | SUCLG1 | UGP2 |
| ARG1 | CCNC | ECOP | GGT1 | IRF9 | MR1 | PKM2 | SAMD9L | SUPT7L | UHMK1 |
| ARHGAP24 | CCND3 | EDN1 | GINS4 | ITGA1 | MS4A6A | PLAU | SAMSN1 | SUSD3 | UNQ5814 |
| ARHGEF17 | CCRL2 | EGR1 | GJB6 | ITGA6 | MSL3L1 | PLCD1 | SAP30 | SYNE1 | UNQ6487 |
| ARID3A | CCRN4L | EGR2 | GK5 | ITGAM | MSRA | PLD1 | SBNO1 | SYNGR2 | UNQ9364 |
| ARID5A | CD14 | EGR3 | GLCCI1 | ITIH4 | MTHFD2 | PLEC1 | SC4MOL | TAZ | UNQ9368 |
| ARL5B | CD177 | EHD1 | GLRX | ITPKC | MTMR6 | PLEK | SCCPDH | TBC1D8 | UPB1 |
| ARMC8 | CD44 | EIF2AK2 | GNA15 | IVNS1ABP | MUC1 | PLK2 | SCN1B | tcag7.1015 | UPP1 |
| ATG10 | CD48 | EIF3I | GPAA1 | JAK2 | MUSTN1 | PLK3 | SCN9A | tcag7.1177 | USP47 |
| ATG7 | CD55 | ELL2 | GPR108 | JMJD6 | MUT | PLSCR1 | SDHC | TCFL5 | VAPA |
| ATM | CD69 | ELMO2 | GPR109A | JOSD1 | MVK | PLSCR4 | SEC22B | TCN1 | VAT1 |
| ATP11B | CD83 | EMB | GPR109B | KBTBD6 | MXD3 | PNPLA1 | SEMA6B | TCP1 | VAV1 |
| ATP13A3 | CDADC1 | EML4 | GPR132 | KBTBD7 | N4BP1 | POLE | SENP2 | TDRD7 | VILL |
| ATP2C2 | CDKN1A | EMR1 | GPR141 | KCNAB3 | NAPRT1 | POR | SEPHS2 | TEAD3 | VNN1 |
| ATP6V1C1 | CEACAM1 | EPB41L5 | GPR160 | KCND1 | NBN | PPP1R12B | SERINC2 | TES | VPS54 |
| ATP8B4 | CEBPZ | EREG | GPR65 | KCNE1 | NCAPD2 | PPP1R15A | SERPINB1 | TESC | VRK1 |
| AXUD1 | CENTB1 | ERLIN1 | GPR84 | KCNJ2 | NCOA7 | PPP2R5A | SERPINB10 | TFDP1 | WDFY3 |
| AZI2 | CENTD1 | ETFDH | GRAMD1A | KLAA0528 | NCR1 | PPP2R5B | SERPINB8 | TFDP2 | WDR32 |
| AZIN1 | CENTG1 | ETS2 | GRAMD4 | KLAA1632 | NDST2 | PPP4R2 | SERPINB9 | TFEC | WDR41 |
| B3GALNT1 | CEP135 | EXOC6 | GRINA | KLF1B | NDUFB3 | PRDM1 | SESN2 | TFF3 | WSB1 |
| B3GNT5 | CFL2 | EXOSC4 | GRPEL1 | KLF3C | NECAP2 | PRDM8 | SFRS12IP1 | TFG | XBP1 |
| B4GALT4 | CH25H | EXT1 | GSDMD | KL | NEDD4 | PRIC285 | SFT2D1 | TFRC | XRCC4 |
| B4GALT5 | CHD1 | EZH2 | GTDC1 | KLF5 | NEIL1 | PRKAR2A | SFXN1 | TGIF2 | XRN1 |
| BATF | CHD4 | F5 | GTPBP1 | KLHL2 | NEK9 | PRKD3 | SFXN5 | THEX1 | YIPF5 |
| BATF3 | CHORDC1 | FAM110B | GTPBP2 | KLHL9 | NFKB1 | PROKR2 | SGMS2 | TICAM1 | ZC3H12A |
| BAZ1A | CHST2 | FAM125B | GYG1 | L3MBTL3 | NFKB2 | PRPSAP1 | SGPP2 | TIFA | ZC3H3 |
| BCL2A1 | CISH | FAM126B | GYS1 | LACTB | NFKBIA | PSMC4 | SH2D3A | TJAP1 | ZDHHC17 |
| BIRC3 | CKAP4 | FAM160A2 | H6PD | LAMB3 | NFKBIE | PSTPIP2 | SH3BP5 | TMED8 | ZDHHC2 |
| BMX | CLEC4D | FAM172A | HBEGF | LCN2 | NFKBIZ | PTGER2 | SH3BP5L | TMEM110 | ZDHHC3 |
| BNIP2 | CLEC5A | FAM177A1 | HCST | LDHA | NIT1 | PTGES | SHB | TMEM120A | ZNF107 |
| BPI | CLIC4 | FAM55C | HDAC4 | LDLR | NLRC4 | PTPN2 | SIGLEC5 | TMEM165 | ZNF250 |
| BST2 | CLTCL1 | FAR1 | HDGF | LGALS1 | NLRC5 | PTPN22 | SKIL | TMEM180 | ZNF254 |
| C10orf119 | CNIH4 | FBXO39 | HDHD2 | LGALS8 | NLRP3 | PTX3 | SLAMF7 | TMEM185B | ZNF271 |
| C10orf30 | COP1 | FBXO6 | HELB | LIG4 | NME6 | PUS10 | SLAMF8 | TMEM205 | ZNF276 |
| C10orf57 | COX7A2L | FBXO9 | HGF | LILRA5 | NOD2 | PUS3 | SLC11A2 | TMEM38A | ZNF277 |
| C10orf97 | CPNE5 | FBXW2 | HIATL1 | LILRB3 | NPAT | PVRL1 | SLC18A2 | TMEM56 | ZNF281 |
| C11orf51 | CR1 | FCAR | HIF1AN | LIMK2 | NR2E1 | PVRL2 | SLC1A3 | TMEM63A | ZNF282 |
| C11orf71 | CRISP3 | FCER1G | HIP1 | LIMS1 | NRAS | PXK | SLC24A3 | TMTC1 | ZNF354A |
| C11orf82 | CRTC3 | FCN1 | HIPK2 | LMNB1 | NSMCE2 | PYROXD1 | SLC25A12 | TNF | ZNF410 |
| C12orf61 | CSGALNACT2 | FCRL1 | HIST1H3E | LMTK2 | NSUN4 | QPCT | SLC25A24 | TNFAIP3 | ZNF43 |
| C13orf18 | CST7 | FEM1C | HIVEP1 | LOC | NSUN6 | QSOX1 | SLC25A40 | TNFAIP6 | ZNF445 |
| C13orf23 | CSTA | FERMT3 | HK1 | 100129395 | NT5DC3 | RAB24 | SLC26A6 | TNFRSF10A | ZNF460 |
| C13orf31 | CSTF2T | FES | HK2 | LOC147804 | NTNG2 | RAB27A | SLC26A8 | TNFRSF10D | ZNF512 |
| C14orf101 | CTSH | FFAR2 | HK3 | LOC284757 | NXT2 | RAB32 | SLC27A2 | TNFSF12- | ZNF595 |
| C15orf48 | CUX1 | FFAR3 | HLX | LOC401152 | OAT | RAB3GAP2 | SLC30A7 | TNFSF13 | ZNF718 |
| C16orf57 | CYB5D1 | FGD4 | HMGB2 | LOXHD1 | ODZ1 | RAB43 | SLC35B2 | TNFSF13B | |
| | | | | LRP3 | | | | TNFSF8 | |

TABLE 44B
Activated neutrophil genes that overlap with genes in Module B

| | | | | | |
|---|---|---|---|---|---|
| ANXA4 | CLEC5A | FBXO9 | HP | MMP8 | SERPINB10 |
| ARG1 | CRISP3 | GCLM | KBTBD6 | OLFM4 | STOM |
| ATP8B4 | CYBB | HMGB2 | LCN2 | OLR1 | TCN1 |

TABLE 44C
Activated neutrophil genes that overlap with genes
in Module B implicated in neutrophil degranulation

| | | | | |
|---|---|---|---|---|
| ARG1 | CRISP3 | LCN2 | OLR1 | TCN1 |
| ATP8B4 | CYBB | MMP8 | SERPINB10 | |
| CLEC5A | HP | OLFM4 | STOM | |

### Example 22: LDG Module B Gene Coexpression Analysis in SLE Blood and Affected Organs

Next, it was determined whether the genes in LDG module B were coexpressed cohesively from patient to patient. WGCNA was used to construct ME for the module B genes in six WB and PBMC gene expression datasets as well as datasets from LN glomerulus and TI, skin, and synovium. The kME was used to assess the quality of the gene expression module in each dataset. To obtain points of reference, COXPRESdb (available at coxpresdb.jp) was queried for genes co-expressed with CD79A (Table 45A) and ZAP70 (Table 45B) and thus associated with BCR signaling and TCR signaling, respectively.

TABLE 45A
Genes co-expressed with CD79A COXPRESdb for co-expression
analysis in SLE blood and affected tissues

| | | | | |
|---|---|---|---|---|
| CD79A | HLA-DOB | FCER2 | FAM129C | LOC100130458 |
| CD19 | TLR9 | MZB1 | LY9 | IGLL3P |
| MS4A1 | SPIB | IGLJ3 | IGLV3-19 | FCRL1 |
| CD79B | BANK1 | FAIM3 | LOC100130100 | IGKV4-1 |
| VPREB3 | TCL1A | CXCR5 | IGKV1OR2-118 | DTX1 |
| CD22 | IGHM | LOC100131043 | TNFRSF17 | IRF4 |
| BLK | STAP1 | DTNB | 90925 | LINC00926 |
| CPNE5 | PNOC | FCRLA | FAM20B | SIT1 |
| POU2AF1 | P2RX5 | FCRL5 | CR2 | IGKC |
| PTPRCAP | TLR10 | 652493 | SEL1L3 | IGLV4-60 |
| CD27 | CD72 | IGL | IGHD | IGLV6-57 |

| | | | | |
|---|---|---|---|---|
| CCR6 | BFSP2 | LAX1 | BTLA | CARD11 |
| MAP4K1 | RHOH | BLNK | LIMD2 | BACH2 |
| SEPT1 | PIM2 | EBF1 | GCSAM | |
| FAM30A | TBC1D10C | WDFY4 | LRMP | |
| CLLU1 | TMEM156 | PVR1G | TSPAN33 | |
| STAG3 | SP140 | CCR7 | SASH3 | |
| CD37 | IGJ | GPR18 | RGS13 | |
| 414332 | FCRL2 | CD180 | LINC00494 | |
| FCRL3 | CD40 | VPREB1 | FDCSP | |
| P2RY10 | PLCG2 | ANKRD36BP2 | LTB | |
| KIAA0125 | P2RY8 | PKHD1L1 | IGHG1 | |

TABLE 45B
Genes co-expressed with ZAP70 in COXPRESdb for co-expression analysis in SLE blood and affected tissues

| | | | | | | | | | |
|---|---|---|---|---|---|---|---|---|---|
| ZAP70 | UBASH3A | TRAC | RASAL3 | CARD11 | KLRK1 | CD8B | DEF6 | CTSW | RASGRP1 |
| LCK | ITK | CXorf65 | ICOS | PATL2 | PYHIN1 | PRKCQ-AS1 | TXK | LAX1 | PLEKHF1 |
| SEPT1 | CD3G | TBC1D10C | CD8A | TESPA1 | THEMIS | GNLY | PBX4 | GIMAP5 | |
| CD247 | PVRIG | ZNF831 | FLT3LG | GZMK | SAMD3 | LOC339988 | PTPN7 | PRKCH | |
| CD3D | CD2 | 100134558 | CD96 | ARL4C | GZMA | KCNA3 | LAG3 | IL7R | |
| CD6 | PTPRCAP | LIME1 | FAIM3 | EOMES | SIT1 | CD28 | MAP4K1 | RHOH | |
| GZMM | SCML4 | BCL11B | TRAT1 | IL32 | ITGAL | SKAP1 | DENND1C | TRBV7-3 | |
| CD7 | TRA | PRF1 | PRKCQ | GPR171 | ACAP1 | SH2D2A | ZNF101 | P2RY10 | |
| TRBC1 | SLA2 | SH2D1A | DGKA | CD27 | SCARNA17 | KLRG1 | GRAP2 | GIMAP1 | |
| CD3E | SPOCK2 | SIRPG | TMC8 | CCR7 | KLRB1 | TIGIT | TRBV7-8 | TRAF3IP3 | |
| CD5 | NLRC3 | IL2RB | DBH-AS1 | CTLA4 | GZMH | IL2RG | TBX21 | GZMB | |

In the original LDG expression data, the module B genes were considered the standard, with a mean kME of 0.67. In blood datasets, the mean kME of the module B genes had a range of 0.41-0.52, with a grand mean of 0.46 (Table 33). This was considered acceptable with regard to the original LDG expression data. For reference, the CD79A (BCR) and ZAP70 (TCR) modules exhibited grand mean kMEs across blood datasets of 0.55 and 0.65, respectively.

In tissue datasets, however, the module B genes had mean kMEs ranging from 0.02 to 0.24, with a grand mean of 0.14, whereas the ZAP70 (TCR) and CD79A (BCR) modules both had grand mean kMEs of 0.72 (Table 34). These results indicate that the module B genes acted as a cohesive module in blood expression data but not in tissue data. This implies that LDGs defined by module B expression are not present in the tissues, but further testing was done to assess this assumption.

TABLE 33
LDG module B genes are coexpressed in SLE blood

| | | CD79A | ZAP70 |
|---|---|---|---|
| | Module B | COXPRESdb | COXPRESdb |
| Study | Mean kME | Mean kME | Mean kME |
| GSE49454 WB | 0.51 | 0.58 | 0.66 |
| GSE88884 ILLUMINATE-1 WB | 0.44 | 0.52 | 0.67 |
| GSE88884 ILLUMINATE-2 WB | 0.41 | 0.51 | 0.66 |
| GSE50772 PBMC | 0.46 | 0.64 | 0.69 |
| GSE81622 PBMC | 0.52 | 0.44 | 0.56 |
| FDABMC3 PBMC | 0.42 | 0.62 | 0.69 |
| Blood grand mean | 0.46 | 0.55 | 0.65 |
| GSE26975 LDGs | 0.67 | NA | NA |

Note:
Mean kMEs, the correlation of each gene in module B with the overall eigengene, are shown for blood gene expression data. Mean kME values in LDG gene expression data are shown for reference as well as mean kME values for genes coexpressed with CD79A and ZAP70 as determined from COXPRESdb.

Module B was further broken down into three submodules of genes based on GO designations to account for the possibility that the noise of the tissue environment could be masking the behavior of the module. The three submodules were made up of neutrophil degranulation genes (Table 43B, columns 1-2), cell cycle genes (Table 43B, column 3), and genes that did not have either designation (other) (Table 43B, columns 4-5). In the original LDG expression data, the neutrophil degranulation, cell cycle, and other submodules had mean kMEs of 0.83, 0.73, and 0.61, respectively. Across tissue datasets, they had grand mean kMEs of 0.15, 0.58, and 0.18, respectively (Table 34). These results show that cell cycle-related genes behave cohesively in the tissues, but the rest of the genes in module B do not, suggesting that cells other than LDGs convey the signature of cell cycle-related genes in lupus tissues. Overall, the gene coexpression results indicate that although LDGs are enriched in the blood of SLE patients, LDGs are not enriched in SLE-affected organs. Because the LDG module B genes were coexpressed in blood but not in tissue, further analyses were carried out to evaluate the presence of LDGs in SLE peripheral blood.

TABLE 34
LDG module B genes are not coexpressed in SLE-affected tissues

| | | | Cell | | | |
|---|---|---|---|---|---|---|
| | Module | Neutrophil | Cycle | Other | CD79A | ZAP70 |
| | B Mean | Degranulation | Mean | Mean | COXPRESdb | COXPRESdb |
| Study | kME | Mean kME | kME | kME | Mean kME | Mean kME |
| GSE32591 | 0.21 | 0.25 | 0.73 | 0.12 | 0.77 | 0.70 |
| kidney | | | | | | |
| glomerulus | | | | | | |
| GSE32591 | 0.24 | 0.10 | 0.47 | 0.27 | 0.50 | 0.47 |
| kidney TI | | | | | | |
| GSE52471 skin | 0.19 | 0.09 | 0.54 | 0.17 | 0.84 | 0.82 |
| GSE72535 skin | 0.05 | 0.04 | 0.55 | 0.23 | 0.81 | 0.89 |
| GSE36700 | 0.02 | 0.29 | 0.62 | 0.09 | 0.66 | 0.74 |
| synovium | | | | | | |
| Tissue grand | 0.14 | 0.15 | 0.58 | 0.18 | 0.72 | 0.72 |
| mean | | | | | | |
| GSE26975 LDGs | 0.67 | 0.83 | 0.73 | 0.61 | NA | NA |

Note:
Mean kMEs, the correlation of each gene in module B with the overall eigengene, are shown for tissue gene expression data. Mean kME values in LDG gene expression data are shown for reference as well as mean kME values for genes coexpressed with CD79A and ZAP70 as determined from COXPRESdb. Submodules based on GO designations display different behaviors from the module as a whole.

### Example 23: Associations Between LDG Module B Enrichment, SLE Disease Manifestations, and Corticosteroid Treatment

To evaluate the presence of LDGs in SLE peripheral blood, GSVA was used to query lupus WB gene expression data from GSE88884 for the enrichment of LDG module B genes in 1612 SLE patients. GSVA was performed separately on the data derived from the two clinical trials (ILLUMINATE-1 and ILLUMINATE-2) contained within this dataset. LDG enrichment was modestly but significantly correlated with increasing SLEDAI (Spearman rho=0.192, p=6.59×10⁻¹⁵). Welch's unequal variances t test was used to determine whether LDG enrichment scores were significantly different in patients with and without each component of the SLEDAI score or patients receiving any of four classes of drugs (Table 35). LDG enrichment was significantly greater in patients with anti-dsDNA seropositivity (p=2.14×10⁻²⁵), those with low serum complement (p=9.02×10⁻²³), and those taking corticosteroids (p=1.26×10⁻³³). LDG enrichment was also greater in patients with hematuria, proteinuria, pyuria, pericarditis, vasculitis, or leukopenia and those taking immunosuppressives (all p<0.05). LDG enrichment was decreased in patients taking nonsteroidal anti-inflammatory drugs (NSAIDs) or antimalarials and those with arthritis or mucosal ulcers (all p<0.05).

TABLE 35
LDG enrichment is associated with treatment,
SLE disease manifestations, and SLEDAI

| | Estimate | t-Statistic | p Value |
|---|---|---|---|
| Antimalarials (n = 1091) | −0.041* | −2.24 | 0.025 |
| Corticosteroids (n = 1184) | 0.212* | 12.6 | 1.26 × 10⁻³³ |
| Immunosuppressants (n = 670) | 0.067* | 3.90 | 1.00 × 10⁻⁴ |
| NSAIDs (n = 506) | −0.086* | −4.83 | 1.59 × 10⁻⁶ |
| Alopecia (n = 982) | −0.001 | −0.06 | 0.956 |
| Anti-dsDNA (n = 953) | 0.174* | 10.6 | 2.14 × 10⁻²⁵ |
| Arthritis (n = 1413) | −0.103* | −4.00 | 8.17 × 10⁻⁵ |
| Fever (n = 31) | 0.091 | 1.33 | 0.195 |
| Hematuria (n = 44) | 0.128* | 2.44 | 0.019 |
| Leukopenia (n = 125) | 0.098* | 3.27 | 1.32 × 10⁻³ |
| Low complement (n = 748) | 0.164* | 9.98 | 9.02 × 10⁻²³ |
| Mucosal ulcers (n = 563) | −0.042* | −2.38 | 0.018 |
| Myositis (n = 17) | −0.067 | −0.83 | 0.420 |
| Pericarditis (n = 28) | 0.140* | 2.23 | 0.034 |
| Pleurisy (n = 110) | 0.018 | 0.53 | 0.597 |
| Proteinuria (n = 46) | 0.145* | 2.81 | 7.18 × 10⁻³ |
| Pyuria (n = 79) | 0.184* | 4.92 | 4.11 × 10⁻⁶ |
| Rash (n = 1133) | 0.020 | 1.10 | 0.272 |
| Thrombocytopenia (n = 29) | 0.087 | 1.23 | 0.230 |
| Vasculitis (n = 119) | 0.137 | 0.67 | 0.549 |
| Visual disturbance (n = 26) | 0.119* | 3.66 | 3.63 × 10⁻⁴ |
| SLEDAI | 0.192* | — | 6.59 × 10⁻¹⁵ |
| (range 6-40, mean 10.4 ± 3.8) | | | |

Note:
“Estimate” denotes the change in LDG enrichment score or Spearman rho (SLEDAI only). Urinary casts, organic brain syndrome, lupus headache, seizure, psychosis, cranial nerve disorder, and cerebrovascular accidents appeared in fewer than five patients each and were excluded from this analysis. Significant estimates (p < 0.05) are bolded and denoted by asterisks.

Based on the results of these tests and trends in the current literature, a smaller panel of characteristics was selected to study in more depth. Corticosteroid treatment was used to divide patients, as it appeared to have strong effects on LDG enrichment. In addition, anti-dsDNA and low complement were selected as manifestations of interest because of their strong associations with LDG enrichment. Vasculitis and the presence of any renal manifestation (proteinuria, hematuria, pyuria, or urinary casts) were also selected for further analysis. Although their p values were modest compared with those of other characteristics, studies may show links between neutrophil-like gene signatures and vasculitis or renal disease in lupus patients.

Welch's unequal variances t test was used to determine whether LDG module B enrichment scores were significantly different in patient subpopulations with and without the manifestations of interest. Gene signatures from plasma cells and cytotoxic T cells (natural killer (NK) cells, NK T (NKT) cells) were used as positive and negative controls, respectively, as plasma cells may be expected to be clearly associated with anti-dsDNA, and cytotoxic cells are not known to be associated with any of the manifestations of interest (Tables 46A-46B).

TABLE 46A
Gene lists used for CD8T/NK/NKT enrichment in SLE WB

| | | | | | | | | |
|---|---|---|---|---|---|---|---|---|
| CD8B | KIR3DL2 | KLRD1 | GZMA | HCST | RASAL3 | KIR2DL1 | KIR2DL5B | KIR2DS4 |
| CRTAM | KLRB1 | KLRF1 | GZMB | CD2 | TIA1 | KIR2DL2 | KIR2DS1 | KIR2DS5 |
| NKTR | KLRC3 | KIR2DL3 | GZMK | CD7 | TXK | KIR2DL4 | KIR2DS2 | KIR3DL3 |
| KIR3DL1 | KLRC4 | GNLY | GZMM | NKG7 | CD8A | KIR2DL5A | KIR2DS3 | KIR3DX1 |

TABLE 46B
Gene lists used for PB/PC enrichment in SLE WB

| | | | | | | | | |
|---|---|---|---|---|---|---|---|---|
| C19orf10 | IGH4-34 | IGLV1-40 | IGHV3-23 | IGLVI-70 | IGHE | IGHV3-21 | IGHV4-28 | IGKV5-2 |
| IGH | IGK | IGLV1-44 | IGLV3-25 | MZB1 | IGHG3 | IGHV3-33 | IGHV4-30-2 | IGLC1 |
| IGHD | IGKC | IGLV2-14 | IGLV4-3 | PRDM1 | IGHM | IGHV3-47 | IGHV4-34 | IGLL3P |
| IGHG1 | IGL | IGLV2-5 | IGH4-28 | THEMIS2 | IGHV1-18 | IGHV3-54 | IGHV5-78 | IGLL5 |
| IGHMBP2 | IGLJ3 | IGLV3-1 | IGLV4-60 | SDC1 | IGHV1-2 | IGHV3-7 | IGKV1D-27 | IGLV3-10 |
| IGHV2-5 | IGLL1 | IGLV3-19 | IGLV5-45 | IGHA1 | IGHV1-46 | IGHV3-72 | IGKV1D-8 | IGLV7-43 |
| IGHV4-31 | IGLV@ | IGHV3-20 | IGLV6-57 | IGHA2 | IGHV3-13 | IGHV3-73 | IGKV4-1 | IGLV9-49 |

When using all patients, all manifestations of interest were significantly associated with increases in the LDG enrichment score (p<0.001) (Table 36). Among corticosteroid users, results closely resembled those acquired with all patients. Among corticosteroid nonusers, anti-dsDNA (p=1.10×10⁻⁴) and low complement (p=3.36×10⁻⁴) remained modestly associated with increased LDG enrichment scores, whereas vasculitis and renal manifestations were no longer associated with increased enrichment scores (p>0.3). Similar tests with other drugs, including antimalarials, immunosuppressives, and NSAIDs, showed that overall associations between LDG enrichment and SLE manifestations were only minimally affected by the presence or absence of these classes of drugs, with the exception of NSAIDs in patients with renal manifestations (Table 39).

TABLE 36
LDG enrichment is associated with different manifestations
depending on corticosteroid treatment

| | Corticosteroids | Estimate | t-Statistic | p Value |
|---|---|---|---|---|
| Anti-dsDNA | All patients | 0.174* | 10.6 | 2.14 × 10⁻²⁵ |
| | Yes | 0.144* | 7.07 | 3.28 × 10⁻¹² |
| | No | 0.111* | 3.92 | 1.10 × 10⁻⁴ |
| Low | All patients | 0.164* | 9.98 | 9.02 × 10⁻²³ |
| complement | Yes | 0.131* | 6.67 | 3.92 × 10⁻¹¹ |
| | No | 0.113* | 3.65 | 3.36 × 10⁻⁴ |
| Renal | All patients | 0.142* | 4.58 | 9.44 × 10⁻⁶ |
| manifestations | Yes | 0.125* | 3.75 | 2.62 × 10⁻⁴ |
| | No | 0.045 | 0.69 | 0.497 |
| Vasculitis | All patients | 0.120* | 3.66 | 3.63 × 10⁻⁴ |
| | Yes | 0.115* | 3.27 | 1.42 × 10⁻³ |
| | No | 0.065 | 0.95 | 0.351 |

Note:
shown are t test results for manifestations of interest in patients grouped by corticosteroid treatment. “Estimate” denotes the change in LDG enrichment score. “Renal manifestations” denote at least one of hematuria, proteinuria, pyuria, or urinary casts. The subset of patients not taking corticosteroids did not show significant differences in LDG enrichment related to renal manifestations or vasculitis. Significant estimates (p < 0.05) are bolded and denoted by asterisks.

TABLE 39
Antimalarials, immunosuppressives, and NSAIDs have little effect on the
associations between LDG enrichment and SLE manifestations of interest

| | Patient Subset | Estimate | t-statistic | p value |
|---|---|---|---|---|
| Anti-dsDNA | All patients | 0.174* | 10.6 | 2.14 × 10−²⁵ |
| | Antimalarials | 0.182* | 9.41 | 3.19 × 10⁻²⁰ |
| | No antimalarials | 0.150* | 4.92 | 1.25 × 10⁻⁶ |
| | Immunosuppressives | 0.165* | 6.24 | 8.70 × 10⁻¹⁰ |
| | No immunosuppressives | 0.174* | 8.38 | 2.01 × 10⁻¹⁶ |
| | NSAIDs | 0.151* | 5.35 | 1.36 × 10⁻⁷ |
| | No NSAIDs | 0.174* | 8.49 | 9.11 × 10⁻¹⁷ |
| Low | All patients | 0.164* | 9.98 | 9.02 × 10⁻²³ |
| Complement | Antimalarials | 0.161* | 8.20 | 6.70 × 10⁻¹⁶ |
| | No antimalarials | 0.167* | 5.59 | 3.67 × 10⁻⁸ |
| | Immunosuppressives | 0.158* | 6.06 | 2.28 × 10⁻⁹ |
| | No immunosuppressives | 0.161* | 7.60 | 7.61 × 10⁻¹⁴ |
| | NSAIDs | 0.172* | 5.66 | 3.22 × 10⁻⁸ |
| | No NSAIDs | 0.149* | 7.44 | 2.01 × 10⁻¹³ |
| Renal | All patients | 0.142* | 4.58 | 9.44 × 10⁻⁶ |
| Manifestations | Antimalarials | 0.122* | 3.19 | 0.002 |
| | No antimalarials | 0.177* | 3.37 | 0.001 |
| | Immunosuppressives | 0.156* | 3.39 | 0.001 |
| | No immunosuppressives | 0.128* | 3.08 | 0.003 |
| | NSAIDs | −0.007 | −0.12 | 0.909 |
| | No NSAIDs | 0.168* | 4.93 | 2.45 × 10⁻⁶ |
| Vasculitis | All patients | 0.120* | 3.66 | 3.63 × 10⁻⁴ |
| | Antimalarials | 0.110* | 2.85 | 0.005 |
| | No antimalarials | 0.132* | 2.21 | 0.031 |
| | Immunosuppressives | 0.140* | 2.74 | 0.008 |
| | No immunosuppressives | 0.100* | 2.41 | 0.018 |
| | NSAIDs | 0.132 | 1.82 | 0.079 |
| | No NSAIDs | 0.107* | 2.93 | 0.004 |

Note:
“Estimate” denotes the change in LDG enrichment score. Significant estimates (p < 0.05) are bolded and denoted by asterisks. “Renal Manifestations” denotes at least one of hematuria, proteinuria, pyuria, or urinary casts. The subset of patients taking NSAIDs did not show significant differences in LDG enrichment related to renal manifestations or vasculitis.

As expected, plasma cell enrichment was strongly associated with anti-dsDNA irrespective of corticosteroid treatment, and cytotoxic T cell (NK cell, NKT cell) enrichment was not associated with any manifestations of interest, save for a mild association with renal manifestations among corticosteroid users (p=0.015) (Tables 39-40).

TABLE 40
PB/PC enrichment scores are not associated
with disease manifestations

| | Corticosteroids | Estimate | t-statistic | p value |
|---|---|---|---|---|
| Anti-dsDNA | All patients | 0.200* | 10.80 | 3.22 × 10⁻²⁶ |
| | Yes | 0.178* | 7.86 | 1.14 × 10⁻¹⁴ |
| | No | 0.218* | 5.89 | 1.03 × 10⁻⁸ |
| Low | All patients | 0.177* | 9.42 | 1.59 × 10⁻²⁰ |
| Complement | Yes | 0.186* | 8.57 | 3.20 × 10⁻¹⁷ |
| | No | 0.095* | 2.27 | 0.024 |
| Renal | All patients | 0.063 | 1.62 | 0.108 |
| Manifestations | Yes | 0.061 | 1.48 | 0.142 |
| | No | −0.021 | −0.19 | 0.852 |
| Vasculitis | All patients | 0.100* | 2.67 | 0.009 |
| | Yes | 0.069 | 1.60 | 0.112 |
| | No | 0.188* | 2.44 | 0.022 |

Note:
t-test results for manifestations of interest in patients grouped by corticosteroid treatment. “Estimate” denotes the change in LDG enrichment score. Significant estimates (p < 0.05) are bolded and denoted by asterisks. “Renal Manifestations” denotes at least one of hematuria, proteinuria, pyuria, or urinary casts. PB: plasmablast; PC: plasma cell.

TABLE 41
LDG Module B differential enrichment is associated with
manifestations of interest by Fisher's exact test

| | | Odds Ratio | | |
|---|---|---|---|---|
| | Corticosteroids | (95% CI) | | p value |
| Anti-dsDNA | All patients | 2.5 | (2.0, 3.1)* | <2.2 × 10⁻¹⁶ |
| | Yes | 2.3 | (1.7, 2.9)* | 1.34 × 10⁻¹⁰ |
| | No | 1.9 | (1.2, 2.9)* | 2.17 × 10⁻³ |
| Low | All patients | 2.5 | (2.1, 3.1)* | <2.2 × 10⁻¹⁶ |
| Complement | Yes | 2.2 | (1.8, 2.9)* | 2.68 × 10⁻¹¹ |
| | No | 2.1 | (1.3, 3.3)* | 1.05 × 10⁻³ |
| Renal | All patients | 2.4 | (1.6, 3.7)* | 9.57 × 10⁻⁶ |
| Manifestations | Yes | 2.3 | (1.4, 3.7)* | 2.47 × 10⁻⁴ |
| | No | 1.6 | (0.6, 4.9) | 0.324 |
| Vasculitis | All patients | 1.9 | (1.2, 2.9)* | 2.07 × 10⁻³ |
| | Yes | 1.9 | (1.2, 3.3)* | 5.85 × 10⁻³ |
| | No | 1.4 | (0.6, 3.5) | 0.517 |

Notes:
Fisher's exact test results in (A) all patients or (B) patients taking or not taking corticosteroids. Significant odds ratios (p < 0.05) are bolded and denoted by asterisks. CI: confidence interval.

### Example 24: Associations Between LDG Module B “Differential Enrichment” and Evidence of Type 1 IFN and TNF Activity

Further analyses of the links between LDG enrichment and disease manifestations among different patient populations were undertaken to determine whether binary (yes/no) enrichment of LDGs could be used as a diagnostic or proxy test for other clinical traits or gene signatures potentially involved in SLE pathogenesis. WB gene expression data from GSE88884, including HC subjects, were analyzed with GSVA as described above, using LDG module B, the IFN gene signature (IGS), and genes induced by TNF (as described by, for example, [Waddell, S. J., S. J. Popper, K. H. Rubins, M. J. Griffiths, P. O. Brown, M. Levin, and D. A. Relman. 2010. “Dissecting interferon-induced transcriptional programs in human peripheral blood cells,” PLoS One 5: e9753], which is hereby incorporated by reference in its entirety). Patients with a z-score greater than 2 relative to controls were considered positive for differential enrichment of the gene signature in question. LDG differential enrichment was compared with available clinical traits and the IGS and TNF signatures in all patients and in the previously mentioned subgroups based on corticosteroid treatment. Testing for associations between LDG differential enrichment and traits was done by Fisher's exact test.

Differential LDG enrichment was found in 55% (891 of 1,612) of SLE patients, IGS differential enrichment was found in 75% (1,216 of 1,612) of patients, and TNF response differential enrichment was found in 44% (704 of 1,612) of patients. Strong associations with LDG differential enrichment were found for IGS and the TNF response in all patients and in both subgroups of patients by Fisher's exact test (p<1×10⁻¹⁰) (Table 37). Remarkably, LDG enrichment and TNF response had the strongest association in patients not taking corticosteroids, with an odds ratio of 8.3. Associations between LDG differential enrichment and clinical traits of interest were similar to those found by t tests, as LDG differential enrichment was not associated with renal manifestations or vasculitis in patients not taking corticosteroids (Tables 44A-44B).

TABLE 37
LDG differential enrichment is associated
with the IGS and genes induced by TNF

| | | Odds Ratio | | |
|---|---|---|---|---|
| | Corticosteroids | (95% CI) | | p Value |
| IGS | All patients | 5.0 | (3.9, 6.6) | <2.2 × 10⁻¹⁶ |
| | Yes | 4.4 | (3.2, 6.1) | <2.2 × 10⁻¹⁶ |
| | No | 5.0 | (3.0, 8.4) | 1.37 × 10⁻¹² |
| TNF | All patients | 7.5 | (5.9, 9.5) | <2.2 × 10⁻¹⁶ |
| | Yes | 7.0 | (5.3, 9.4) | <2.2 × 10⁻¹⁶ |
| | No | 8.3 | (5.2, 13.4) | <2.2 × 10⁻¹⁶ |

Note:
Fisher's exact test results in patients grouped by corticosteroid treatment. CI, confidence interval.

### Example 25: Comparison of LDG Module B to Immature Bone Marrow PMN Precursor Gene Expression

Samples from GSE19556 were used to compare PM, MY, and bone marrow polymorphonuclear neutrophils (bmPMN) to peripheral blood polymorphonuclear neutrophils (pbPMN) by DE analysis (Table 38). A total of 68 of the 92 genes in LDG module B were differentially expressed in promyelocytes (PM) (overlap p value=1.4×10⁻⁶) compared with 71 in myelocytes (MY) (p=1.8⁻¹⁸) and 28 in bmPMN (p=8.5×10⁻¹²). In contrast, bmPMN did not differentially express the cell cycle portion of module B found in PM and MY, indicating that LDGs are transcriptionally similar to these more immature precursors.

TABLE 38
LDG module B is enriched in neutrophil precursors
normally found in the bone marrow

| | | Upregulated | | Neutrophil | |
|---|---|---|---|---|---|
| | Upregulated | Genes in | | Degranulation | Cell Cycle |
| | Genes versus | LDG | Overlap | Genes in | Genes in |
| Cell Type | pbPMN | Module B | p Value | Overlap | Overlap |
| PM | 4951 | 68 of 92 | 1.4 × 10⁻⁶ | 25 of 30 | 18 of 21 |
| MY | 3267 | 71 of 92 | 1.8 × 10⁻¹⁸ | 28 of 30 | 18 of 21 |
| bmPMN | 690 | 28 of 92 | 8.5 × 10¹² | 20 of 30 | 2 of 21 |

Note:
Overlap p values were calculated using Fisher's exact test and a universe of 10,000 genes to account for the fact that low-intensity genes were filtered out in both experiments, _and this results in more conservative p values than the use of 20,000 genes. bmPMN differentially express only a small portion of the cell cycle signature compared with PM and MY. “pbPMN” denotes peripheral blood PMN.

### Example 26: Genome Documentation of Molecular Pathways Driving the Autoimmune Disease Systemic Lupus Erythematosus by Cross Referencing Signaling Checkpoints in the Immune System Identified in Patients with Primary Immunodeficiency

Systemic lupus erythematosus (SLE) may be a polygenic autoimmune disease defined by hyper-reactivity of the immune system. In healthy individuals, the immune system may protect the host from invading microorganisms. However, subjects (e.g., patients) with primary immunodeficiency (PID) may not be able to generate an effective immune response and hence may suffer from repeated infections. To examine checkpoints in the immune system driving autoimmunity in SLE, sets of genes abnormally expressed in SLE cells were compared to sets of causal genes underlying PID. A hypothesis that genes “knocked out” in PID are overexpressed in lupus, and therefore possibly contributing to the immune over-reactivity, was tested. After compiling a comprehensive database of the 450 genes discovered through this process, at least 388 of the PID-associated genes were observed to be differentially expressed (DE) in SLE. Further, at least 206 of the PID-associated genes were found to be uniquely DE in immune subsets (myeloid, T cells, NK cells, B cells, plasma cells, and neutrophils). A variety of bioinformatics tools were employed to elucidate the nature of the PID-associated genes that were over-expressed in SLE. For example, STRING, a protein-protein interaction analytic tool, was applied to the dataset, and 17 groups (e.g., clusters) of PID-associated genes were identified. Further, Gene Set Variation Analysis (GSVA) was applied to the dataset, and 12 gene clusters were identified to be enriched in a set of 1,620 SLE patients. Notably, clusters of PID-associated genes were consistently enriched (interferon stimulated genes, MHC class-1 antigen presentation, secreted-immune, secreted extracellular matrix, pattern recognition receptors, proteasome activity, and pro-apoptosis). These results demonstrate that the non-redundant checkpoint genes underlying PID are over-expressed in SLE patients. These genes and the pathways they identify may be used as unique targets for novel therapies in SLE.

The results obtained may provide a deeper understanding of the relationship between primary immunodeficiency (PID) genes and a specific autoimmune disorder, systemic lupus erythematosus (SLE). SLE is a complex genetically-based autoimmune disease defined by the production of high affinity autoantibodies that cause damage to tissues and may be lethal. SLE may disproportionately affect certain groups of subjects (e.g., patients), such as females of African ancestry, and may include exacerbations and great variability. PID may be considered as essentially the functional inactivation of the immune system, in which the causal genes are biological upstream regulators. If a particular gene is knocked out in a subject, then a severe immune phenotype may persist, and the subject's susceptibility to recurrent infections may increase significantly. On the other hand, autoimmunity generally arises in a subject from the over-activation of the immune system of the subject. Therefore, PID and autoimmunity may be considered as opposite sides of the same coin.

In some cases, PID and autoimmunity may share the loss of regulatory checkpoints in the immune system, and these checkpoints may be governed by the same genes. Instead of examining the entire human genome, identified PID-associated genes were analyzed, and their role in SLE was elucidated. For example, PID-associated genes may be identified and the role of these genes in SLE may be analyzed, e.g., by cross-referencing differential expression datasets and utilizing various analytical tools to understand the common genes between SLE and PID. Due to the complexity of SLE, many types of drugs (e.g., antimalarial, corticosteroids, immunosuppressants, biologics, and nonsteroidal anti-inflammatory drugs) may be utilized to treat symptoms. Belimumab (Benlysta®), the only drug approved in 60 years to treat SLE, is a biologic that inhibits the binding of B cells to B lymphocyte stimulators. Identified PID-associated genes that are also marker genes for SLE may be explored as potential drug therapy targets for SLE patients.

The PID gene database was constructed as follows. Once identified via thorough searches of primary scientific literature on PIDs, a plurality of causal genes was compiled into a database that includes the following information for each gene: Gene Symbol, Official Symbol, Full Name, Functional Category (BIG-C™), Entrez ID, Ensembl ID, Gene Type, Synonyms, Chromosome Number, Cytogenetic Location, Inheritance, genetic Defect/Pathogenesis, Phenotype, Relevance to SLE, Allelic Mutations (OMIM and Primary literature), Protein Effect (GeneCards), OMIM Gene ID, OMIM Phenotype ID, and Mendelian Genetics ID.

BIG-C™ analysis was performed on the data as follows. Biologically Informed Gene Clustering (BIG-CTM) is a functional aggregating tool (AMPEL BioSolutions, Charlottesville, Va.) for analyzing and understanding the biological groupings of large lists of genes. Genes are sorted into 45 categories based on their most likely biological function and/or cellular localization based on information from multiple online tools and databases.

I-SCOPE analysis was performed on the data as follows. PID-associated genes were cross-referenced with immune genes restrictively expressed in hematopoietic genes restrictively expressed in hematopoietic cells using the I-SCOPE tool (AMPEL BioSolutions, Charlottesville, Va.).

Cytoscape, STRING, and MCODE analyses were performed on the data as follows. A visualization of protein-protein interactions and relationships between genes within datasets was performed using the Cytoscape (V3.6.0) software and the MCODE StringApp (V1.3.2) plugin application. The Clustermaker2 App (V1.2.1) plugin was used to create clusters of the most related genes within a dataset, using a network scoring degree cutoff of 2 and setting a node score cut-off of 0.2, k-Core of 2, and a max depth of 100.

Gene expression data was compiled from SLE patients as follows. Data were derived from publicly available datasets and collaborators. Raw data files were obtained from the GEO repository for SLE whole blood data. The following datasets were used: GSE22098, GSE39088, GSE88884, GSE45291, and GSE61635.

The data was analyzed for differential gene expression (e.g., between SLE patients vs. controls) as follows. GCRMA normalized expression values were variance corrected using local empirical Bayesian shrinkage, followed by calculation of DE using the ebayes function in the BioConductor LIMMA package. Resulting p-values were adjusted for multiple hypothesis testing and filtered to retain DE probes with an FDR<0.2.

Gene Set Variation Analysis (GSVA) was performed on the data as follows. The GSVA (V1.25.0) software package for R/Bioconductor was used as a non-parametric, unsupervised method for estimating the variation of pre-defined gene sets in patient and control samples of microarray expression data sets. GSVA was run using GSE88884 and the MCODE Clusters.

Hedge's G values, a measure of effect size, were calculated from the GSVA enrichment scores, by contrasting K-S scores of all controls against all lupus patient samples. GSVA enrichment scores were additionally utilized for Welch's t-tests to identify significant (e.g., p<0.05) gene categories contributing to substantial segregation of cohort samples. Results were visualized by using a matrix of Hedge's G values was entered as input to the corplot package of R (dual scale heatmap). Significant categories are denoted by asterisks.

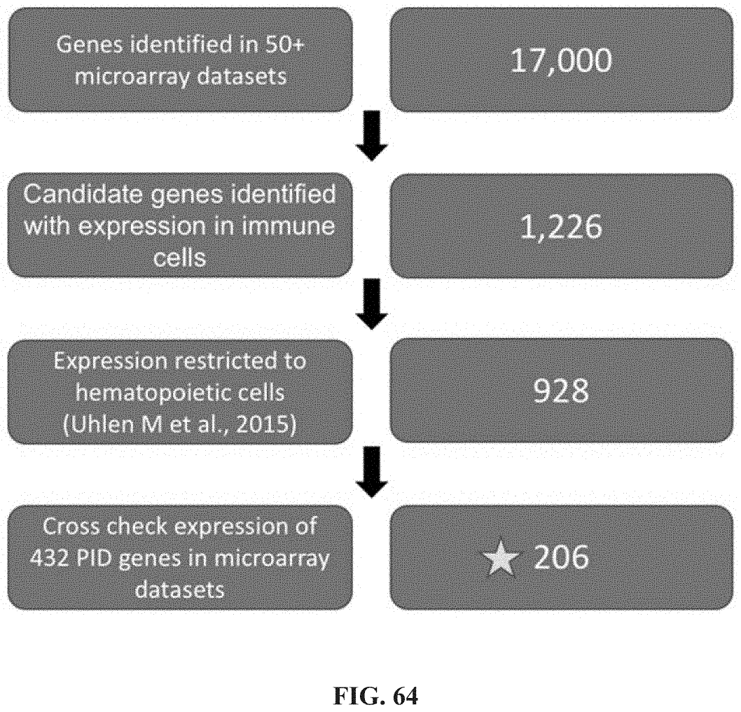

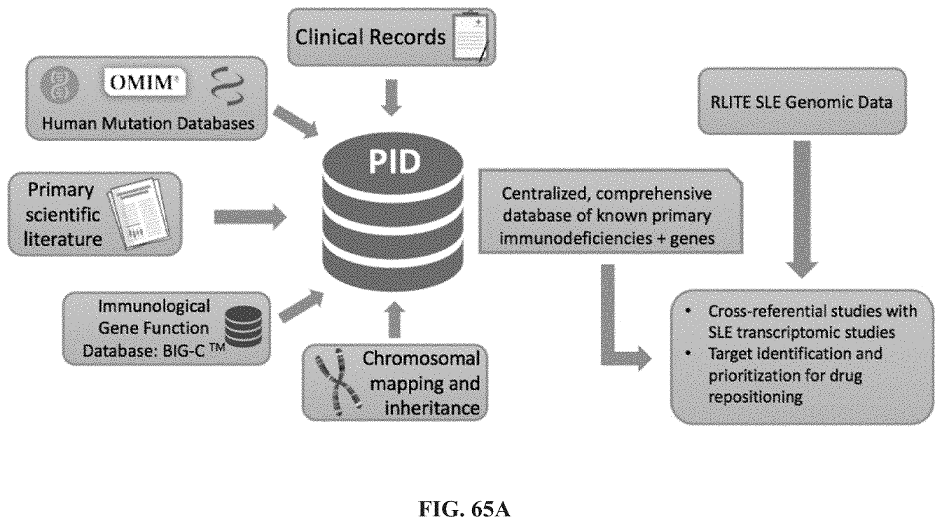

FIG. 64 shows a non-limiting example of cross-checking primary immunodeficiency (PID) genes in 928 hematopoietic immune cells, in accordance with disclosed embodiments. The expression of the genes must be specific to hematopoietic cells, because if not restricted, then these genes could be targeted in non-immune specific cells and have detrimental effects.

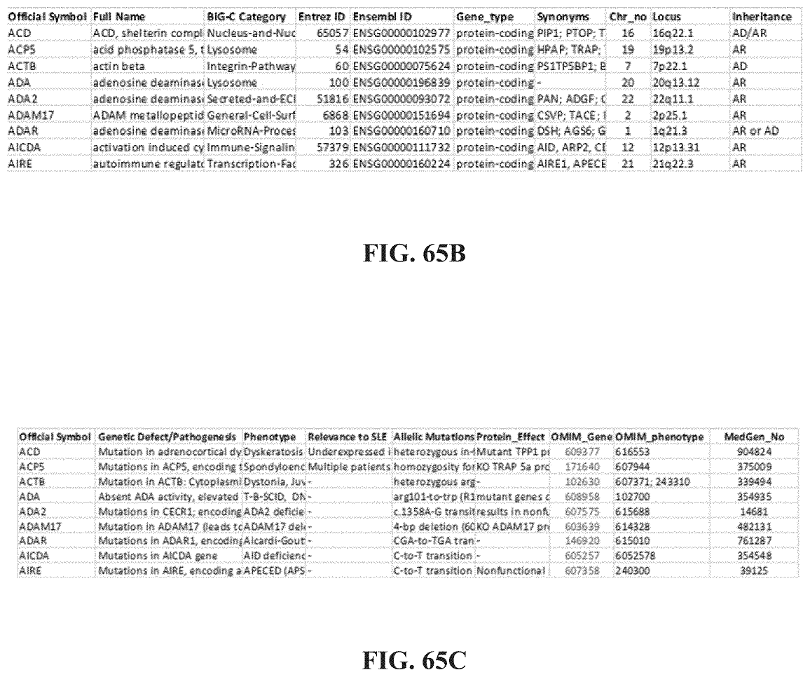

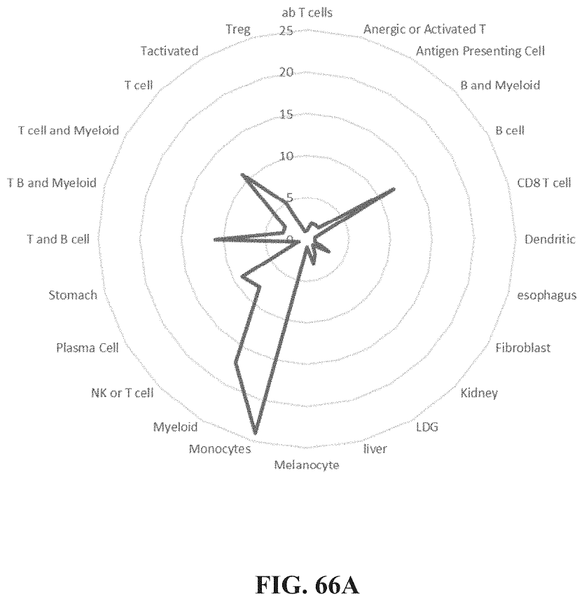

FIG. 65A shows a non-limiting example of a database at large, comprising 432 genes, in accordance with disclosed embodiments. Via deliberation of various primary literature, the database was compiled with 432 PID-associated genes. Each PID gene includes characteristic information that can be used to identify and describe the gene.

FIGS. 65B-65C show a non-limiting example of a table of the database shown in FIG. 65A, in accordance with disclosed embodiments.

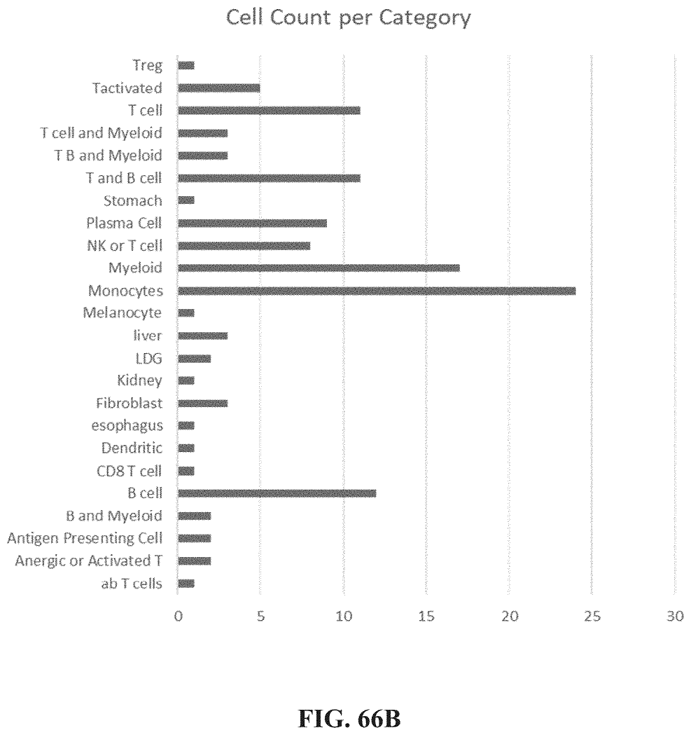

FIG. 66A shows a non-limiting example of results showing that some PID-associated genes are specific to immune hematopoietic stem cells, in accordance with disclosed embodiments. Of the 450 PID-associated genes, 125 genes were determined to be specific to immune hematopoietic cells. Of the 25 immune cell categories specific to hematopoietic cells and various cell types, the 125 genes are concentrated in monocyte, myeloid, B cell, T cell, and B and T cell categories.

FIG. 66B shows a non-limiting example of results showing the cell count per category of various cell types.

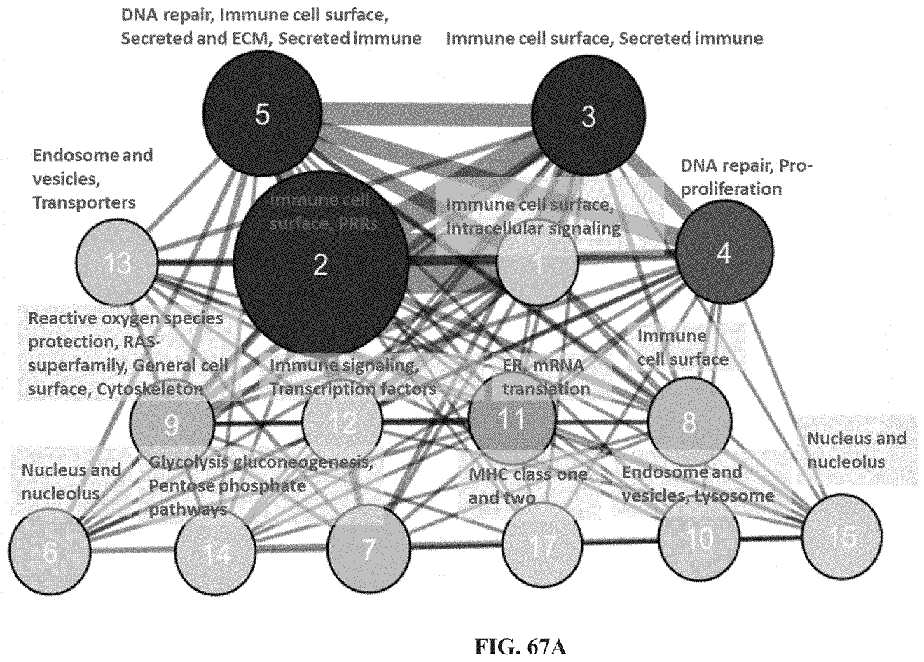

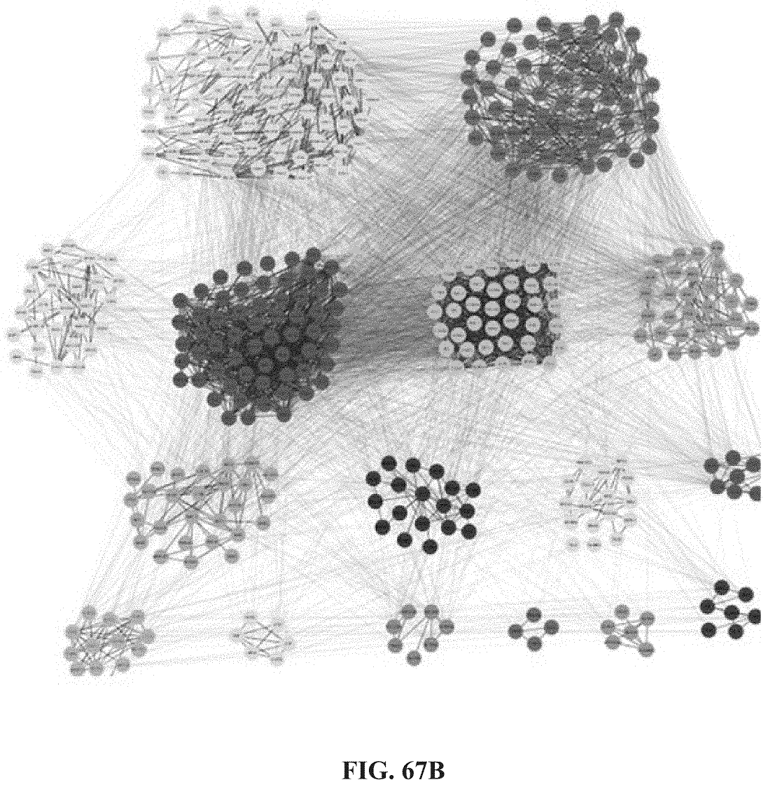

FIGS. 67A-67B show a non-limiting example of protein-protein interaction-based clustering of 450 PID-associated genes, in accordance with disclosed embodiments. Protein-protein interaction networks and clusters were generated via Cytoscape using the STRING and MCODE plugins. FIG. 67A shows that of the 450 genes, 430 genes were grouped into 16 clusters, and the BIG-C™ category most representative of the gene list was used to biologically characterize the clusters. The clusters with the most genes include clusters 1, 2, 3, 4, and 5. The BIG-C™ categories represented by these large clusters include immune cell surface, intracellular signaling, pattern recognition receptors, DNA repair, pro-proliferation, secreted immune, and extracellular matrix. The node sizes correlate to the number of genes in each cluster, and the degree of node shading indicates the number of intracluster connections (see gradient at bottom of figure). The edge weight thickness represents the number of intercluster connections. FIG. 67B shows that the 450 genes were grouped into 16 clusters. Data from GSE88884, which includes transcriptomic data of 1,620 patients, was used to determine the differential expression of the genes.

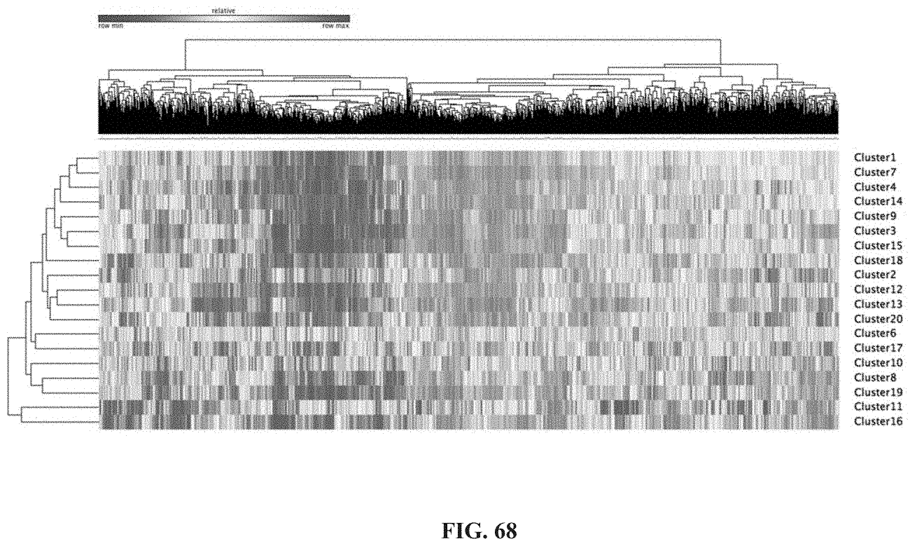

FIG. 68 shows a non-limiting example of endotypes of SLE patients defined by functional groupings of PID-associated genes, in accordance with disclosed embodiments. Differentially expressed (DE) genes from the GSE88884 SLE WB dataset (1,620 patients) were assessed by GSVA for the 17 MCODE clusters, as shown in FIGS. 67A-67B (and on the x-axis of the heatmap). There is a clear distinction between enrichment of the clusters among the patients, thereby demonstrating that these groups of immune-specific genes can be used to differentitate SLE patients based on clinical presentation of disease.

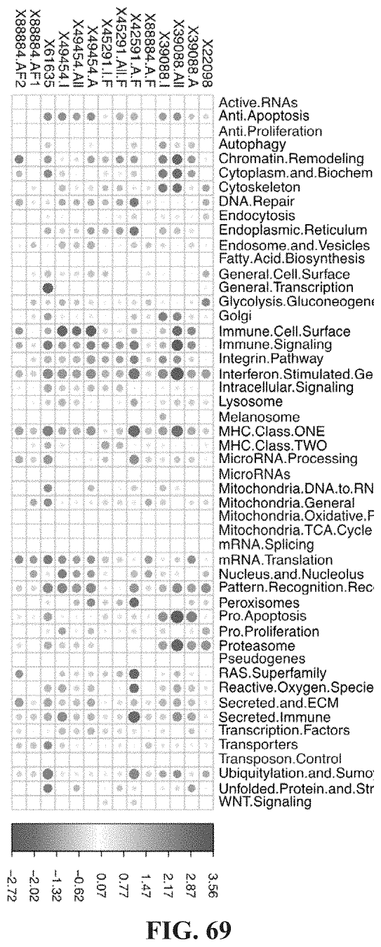

FIG. 69 shows a non-limiting example of performing GSVA to identify the functional role of PID-associated genes expressed in SLE WB microarray datasets, in accordance with disclosed embodiments. DE genes from 14 SLE WB datasets shown on the x-axis were overlapped with the 450 PID-associated genes to assess common genes. SLE WB DE genes that are also PID-associated genes were analyzed by GSVA for function by enrichment with BIG-C functional categories as shown on the y-axis. Welch's t test was used to identify significant BIG-C categories including interferon stimulated genes, MHC class-1 antigen presentation, secreted-immune, secreted extracellular matrix, pattern recognition receptors, proteasome activity, and pro-apoptosis.

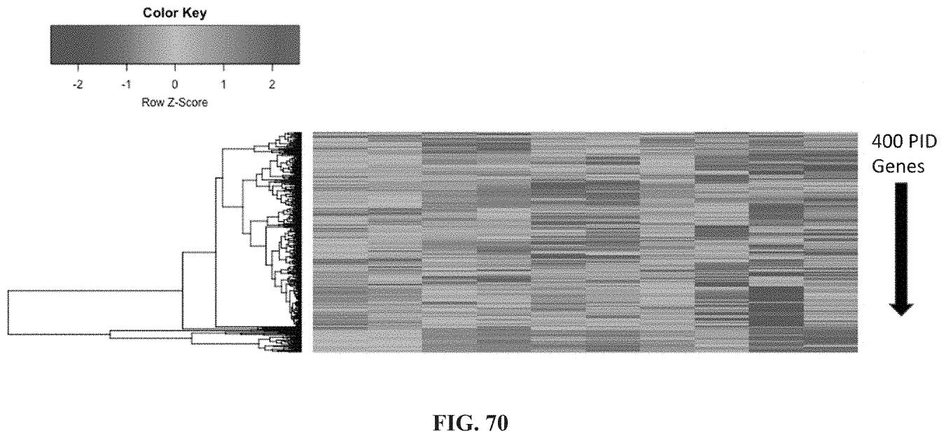

FIG. 70 shows a non-limiting example of results demonstrating that PID-associated genes differentially expressed in a large whole blood dataset comprised of distinct patient groups, in accordance with disclosed embodiments.

Table 47 show a database of 450 PID-associated genes, which were curated from primary literature. These PID-associated genes can be used as marker genes for PID and/or lupus conditions (e.g., based on DE analysis relative to controls).

TABLE 47
PID-associated genes

| Gene | Official | | | Entrez | Relevance |
|---|---|---|---|---|---|
| Symbol | Symbol | Full Name | BIG-C Category | ID | to SLE |
| ACD | ACD | ACD, shelterin complex | Nucleus-and-Nucleolus | 65057 | Underexpressed in |
| | | subunit and telomerase | | | PBMCs from SLE |
| | | recruitment factor | | | patients, negative |
| | | | | | correlation to SLEDAI |
| ACP5 | ACP5 | acid phosphatase 5, | Lysosome | 54 | Multiple patients |
| | | tartrate resistant | | | reported with this |
| | | | | | mutation manifested |
| | | | | | SLE |
| ACTB | ACTB | actin beta | Integrin-Pathway | 60 | — |
| ADA | ADA | adenosine deaminase | Lysosome | 100 | — |
| ADA2 | ADA2 | adenosine deaminase 2 | Secreted-and-ECM | 51816 | — |
| ADAM 17 | ADAM 17 | ADAM metallopeptidase domain 17 | General-Cell-Surface | 6868 | — |
| ADAR | ADAR | adenosine deaminase, | MicroRNA-Processing | 103 | — |
| | | RNA specific | | | |
| AICDA | AICDA | activation induced | Immune-Signaling | 57379 | — |
| | | cytidine deaminase | | | |
| AIRE | AIRE | autoimmune regulator | Transcription-Factors | 326 | — |
| AK2 | AK2 | adenylate kinase 2 | Pro-Apoptosis | 204 | — |
| AP1S3 | AP1S3 | adaptor related protein | Endosome-and-Vesicles | 130340 | — |
| | | complex 1 sigma 3 subunit | | | |
| AP3B1 | AP3B1 | adaptor related protein | Endosome-and-Vesicles | 8546 | — |
| | | complex 3 beta 1 subunit | | | |
| AP3D1 | AP3D1 | adaptor related protein | Endosome-and-Vesicles | 8943 | — |
| | | complex 3 delta 1 subunit | | | |
| APOL1 | APOL1 | apolipoprotein L1 | Secreted-and-ECM | 8542 | — |
| ARPC1B | ARPC1B | actin related protein 2/3 | Endocytosis | 10095 | — |
| | | complex subunit 1B | | | |
| ATM | ATM | ATM serine/threonine kinase | Immune-Signaling | 472 | — |
| ATP6AP1 | ATP6AP1 | ATPase H+ transporting | Lysosome | 537 | — |
| | | accessory protein 1 | | | |
| ATP6V0A2 | ATP6V0A2 | ATPase H+ transporting | Lysosome | 23545 | — |
| | | V0 subunit a2 | | | |
| B2M | B2M | beta-2-microglobulin | MHC-Class-ONE | 567 | — |
| BACH2 | BACH2 | BTB domain and CNC homolog 2 | Immune-Signaling | 60468 | — |
| BCL10 | BCL10 | B-cell CLL/lymphoma 10 | Pro-Apoptosis | 8915 | —- |
| BCL11B | BCL11B | B-cell CLL/lymphoma 11B | Immune-Signaling | 64919 | — |
| BLM | BLM | Bloom syndrome RecQ like | Pro-Proliferation | 641 | Patients may also |
| | | helicase | | | present with systemic |
| | | | | | lupus erythematosus |
| BLNK | BLNK | B-cell linker | Immune-Signaling | 29760 | Patients may also |
| | | | | | present with systemic |
| | | | | | lupus erythematosus |
| BLOC1S6 | BLOC1S6 | biogenesis of lysosomal | Lysosome | 26258 | Patients may also |
| | | organelles complex 1 subunit 6 | | | present with systemic |
| | | | | | lupus erythematosus |
| BTK | BTK | Bruton tyrosine kinase | Immune-Signaling | 695 | Patients may also |
| | | | | | present with systemic |
| | | | | | lupus erythematosus |
| C1QA | C1QA | complement C1q A chain | Secreted-Immune | 712 | Patients may also |
| | | | | | present with systemic |
| | | | | | lupus erythematosus |
| C1QB | C1QB | complement C1q B chain | Secreted-Immune | 713 | Patients may also |
| | | | | | present with systemic |
| | | | | | lupus erythematosus |
| C1QC | C1QC | complement C1q C chain | Secreted-Immune | 714 | Patients may also |
| | | | | | present with systemic |
| | | | | | lupus erythematosus |
| C1R | C1R | complement C1r | Secreted-Immune | 715 | risk of SLE disease |
| | | | | | susceptibility |
| | | | | | significantly increased |
| C1S | C1S | complement C1s | Secreted-Immune | 716 | risk of SLE disease |
| | | | | | susceptibility |
| | | | | | significantly increased |
| C2 | C2 | complement C2 | Secreted-Immune | 717 | — |
| C3 | C3 | complement C3 | Secreted-Immune | 718 | — |
| C4A | C4A | complement C4A | Secreted-Immune | 720 | — |
| | | (Rodgers blood group) | | | |
| C4B | C4B | complement C4B | Secreted-Immune | 721 | — |
| | | (Chido blood group) | | | |
| C4BPA | C4BPA | complement component 4 | Secreted-Immune | 722 | — |
| | | binding protein alpha | | | |
| C4BPB | C4BPB | complement component 4 | Secreted-Immune | 725 | — |
| | | binding protein beta | | | |
| C5 | C5 | complement C5 | Secreted-Immune | 727 | — |
| C6 | C6 | complement C6 | Secreted-Immune | 729 | — |
| C7 | C7 | complement C7 | Secreted-Immune | 730 | — |
| C8A | C8A | complement C8 alpha chain | Secreted-Immune | 731 | — |
| C8B | | | | 732 | — |
| C8G | | | | 733 | — |
| C9 | C9 | complement C9 | Secreted-Immune | 735 | — |
| CARD11 | CARD11 | caspase recruitment | Intracellular-Signaling | 84433 | — |
| | | domain family member 11 | | | |
| CARD14 | CARD14 | caspase recruitment | Anti-Apoptosis | 79092 | — |
| | | domain family member 14 | | | |
| CARD9 | CARD9 | caspase recruitment | Pattern-Recognition- | 64170 | — |
| | | domain family member 9 | Receptors | | |
| CARMIL2 | CARMIL2 | capping protein regulator | Cytoskeleton | 146206 | — |
| | | and myosin 1 linker 2 | | | |
| CASP10 | CASP10 | caspase 10 | Pro-Apoptosis | 843 | — |
| CASP8 | CASP8 | caspase 8 | Pro-Apoptosis | 841 | — |
| CBL | CBL | Cbl proto-oncogene | Ubiquitylation- | 867 | — |
| | | | and-Sumoylation | | |
| CCBE1 | | | | 147372 | — |
| CCDC40 | | | | 55036 | — |
| CCL2 | CCL2 | C-C motif chemokine ligand 2 | Secreted-Immune | 6347 | — |
| CCL22 | CCL22 | C-C motif chemokine ligand 22 | Secreted-Immune | 6367 | — |
| CD19 | CD19 | CD19 molecule | Immune-Cell-Surface | 930 | — |
| CD247 | CD247 | CD247 molecule | Immune-Cell-Surface | 919 | — |
| CD27 | CD27 | CD27 molecule | Immune-Cell-Surface | 939 | |
| CD3D | CD3D | CD3d molecule | Immune-Cell-Surface | 915 | — |
| CD3E | CD3E | CD3e molecule | Immune-Cell-Surface | 916 | — |
| CD3G | CD3G | CD3g molecule | Immune-Cell-Surface | 917 | — |
| CD40 | CD40 | CD40 molecule | Immune-Cell-Surface | 958 | — |
| CD40LG | CD40LG | CD40 ligand | Immune-Cell-Surface | 959 | Currently in Phase |
| | | | | | 3 for treatting RA |
| | | | | | by Pfizer-compound |
| | | | | | palbociclib |
| CD46 | CD46 | CD46 molecule | General-Cell-Surface | 4179 | |
| CD55 | CD55 | CD55 molecule | General-Cell-Surface | 1604 | — |
| | | (Cromer blood group) | | | |
| CD59 | CD59 | CD59 molecule | General-Cell-Surface | 966 | — |
| | | (CD59 blood group) | | | |
| CD70 | CD70 | CD70 molecule | Immune-Cell-Surface | 970 | — |
| CD79A | CD79A | CD79a molecule | Immune-Cell-Surface | 973 | — |
| CD79B | CD79B | CD79b molecule | Immune-Cell-Surface | 974 | — |
| CD81 | CD81 | CD81 molecule | Immune-Cell-Surface | 975 | — |
| CD8A | CD8A | CD8a molecule | Immune-Cell-Surface | 925 | — |
| CDCA7 | CDCA7 | cell division cycle | Intracellular-Signaling | 83879 | — |
| | | associated 7 | | | |
| CDK4 | CDK4 | Cyclin Dependent Kinase 4 | n/a | 1019 | Patient with CFI |
| | | | | | deficiency developed |
| | | | | | SLE |
| CDK6 | CDK6 | Cyclin Dependent Kinase 6 | Pro-proliferation | 1021 | — |
| CEBPE | CEBPE | CCAAT/enhancer | mRNA-Translation | 1053 | |
| | | binding protein epsilon | | | |
| CFB | CFB | complement factor B | Secreted-and-ECM | 629 | — |
| CFD | CFD | complement factor D | Secreted-and-ECM | 1675 | |
| CFH | CFH | complement factor H | Secreted-and-ECM | 3075 | — |
| CFHR1 | CFHR1 | complement factor H related 1 | Secreted-and-ECM | 3078 | — |
| CFHR2 | CFHR2 | complement factor H related 2 | Secreted-and-ECM | 3080 | — |
| CFHR3 | CFHR3 | complement factor H related 3 | Secreted-and-ECM | 10878 | — |
| CFHR4 | CFHR4 | complement factor H related 4 | Secreted-and-ECM | 10877 | — |
| CFHR5 | CFHR5 | complement factor H related 5 | Secreted-and-ECM | 81494 | Mutation found in |
| | | | | | lupus-related trait |
| | | | | | locus |
| CFI | CFI | complement factor I | Secreted-and-ECM | 3426 | Increased |
| | | | | | susceptibility |
| | | | | | to SLE |
| CFP | CFP | complement factor properdin | Secreted-and-ECM | 5199 | — |
| CFTR | CFTR | cystic fibrosis transmembrane | Transporters | 1080 | |
| | | conductance regulator | | | |
| CHD7 | CHD7 | chromodomain helicase | Chromatin-Remodeling | 55636 | — |
| | | DNA binding protein 7 | | | |
| CIB1 | CIB1 | calcium and integrin binding 1 | DNA-Repair | 10519 | Increased |
| | | | | | susceptibility to SLE |
| CIITA | CIITA | class II major | MHC-Class-TWO | 4261 | — |
| | | histocompatibility | | | |
| | | complex transactivator | | | |
| CLCN7 | CLCN7 | chloride voltage-gated | Lysosome | 1186 | — |
| | | channel 7 | | | |
| CLEC7A | CLEC7A | C-type lectin domain | General-Cell-Surface | 64581 | — |
| | | containing 7A | | | |
| CLPB | CLPB | ClpB homolog, mitochondrial | Mitochondria-General | 81570 | — |
| | | AAA ATPase chaperonin | | | |
| COLEC11 | COLEC11 | collectin subfamily member 11 | Secreted-and-ECM | 78989 | — |
| COPA | COPA | coatomer protein complex | Golgi | 1314 | — |
| | | subunit alpha | | | |
| CORO1A | CORO1A | coronin 1A | Cytoskeleton | 11151 | — |
| CR2 | CR2 | complement C3d receptor 2 | Immune-Cell-Surface | 1380 | — |
| CSF2RA | CSF2RA | colony stimulating factor | Immune-Cell-Surface | 1438 | OMIM SLE phenotype |
| | | 2 receptor alpha subunit | | | |
| CSF2RB | CSF2RB | colony stimulating factor | Immune-Cell-Surface | 1439 | high SLE disease |
| | | 2 receptor beta common subunit | | | activity index |
| | | | | | resulted in increase |
| | | | | | in DNMT |
| CSF3R | CSF3R | colony stimulating factor | General-Cell-Surface | 1441 | — |
| | | 3 receptor | | | |
| CTC1 | CTC1 | CST telomere replication | Nucleus-and-Nucleolus | 80169 | — |
| | | complex component 1 | | | |
| CTLA4 | CTLA4 | cytotoxic T-lymphocyte | Immune-Cell-Surface | 1493 | — |
| | | associated protein 4 | | | |
| CTPS1 | CTPS1 | CTP synthase 1 | Cytoplasm-and-Biochemistry | 1503 | — |
| CTSC | CTSC | cathepsin C | Lysosome | 1075 | — |
| CXCR4 | CXCR4 | C-X-C motif chemokine | Immune-Cell-Surface | 7852 | |
| | | receptor 4 | | | |
| CYBA | CYBA | cytochrome b-245 alpha chain | Reactive-Oxygen- | 1535 | |
| | | | Species-Protection | | |
| CYBB | CYBB | cytochrome b-245 beta chain | Reactive-Oxygen- | 1536 | — |
| | | | Species-Protection | | |
| DCLRE1B | DCLRE1B | DNA cross-link repair 1B | DNA-Repair | 64858 | — |
| DCLRE1C | DCLRE1C | DNA cross-link repair 1C | DNA-Repair | 64421 | — |
| DKC1 | DKC1 | dyskerin pseudouridine | Nucleus-and-Nucleolus | 1736 | — |
| | | synthase 1 | | | |
| DNA2 | DNA2 | DNA Replication Helicase/Nuclease 2 | | 1763 | |
| DNAI1 | DNAI1 | Dynein Axonemal Intermediate Chain 1 | | 27019 | |
| DNAJC21 | DNAJC21 | DnaJ heat shock protein | Unfolded-Protein- | 134218 | candidate |
| | | family (Hsp40) member C21 | and-Stress | | contributory |
| | | | | | gene in SLE |
| DNASE1L3 | DNASE1L3 | deoxyribonuclease 1 like 3 | Nucleus-and-Nucleolus | 1776 | |
| DNASE2 | DNASE2 | deoxyribonuclease 2, lysosomal | Lysosome | 1777 | — |
| DNMT3B | | | | 1789 | — |
| DOCK2 | DOCK2 | dedicator of cytokinesis 2 | Cytoskeleton | 1794 | Genetic defect found |
| | | | | | more frequently in |
| | | | | | patients with SLE |
| DOCK8 | DOCK8 | dedicator of cytokinesis 8 | Pattern-Recognition- | 81704 | — |
| | | | Receptors | | |
| DSP | DSP | desmoplakin | Cytoskeleton | 1832 | — |
| ELANE | ELANE | elastase, neutrophil expressed | Secreted-Immune | 1991 | — |
| ELF4 | ELF4 | E74 like ETS | Transcription-Factors | 2000 | — |
| | | transcription factor 4 | | | |
| EPG5 | EPG5 | ectopic P-granules autophagy | Autophagy | 57724 | — |
| | | protein 5 homolog | | | |
| ERBIN | ERBIN | erbb2 interacting protein | Intracellular-Signaling | 55914 | — |
| ERCC6L2 | ERCC6L2 | ERCC excision repair 6 like 2 | DNA-Repair | 375748 | — |
| EXTL3 | EXTL3 | exostosin like | Endoplasmic-Reticulum | 2137 | — |
| | | glycosyltransferase 3 | | | |
| F12 | F12 | coagulation factor XII | Secreted-and-ECM | 2161 | — |
| FAAP24 | FAAP24 | Fanconi anemia core complex | DNA-Repair | 91442 | |
| | | associated protein 24 | | | |
| FADD | FADD | Fas associated via death domain | Pro-Apoptosis | 8772 | — |
| FANCA | FANCA | Fanconi anemia | DNA-Repair | 2175 | |
| | | complementation group A | | | |
| FANCC | FANCC | Fanconi anemia | DNA-Repair | 2176 | — |
| | | complementation group C | | | |
| FANCE | FANCE | Fanconi anemia | DNA-Repair | 2178 | SNPin IFIH1 |
| | | complementation group E | | | associated with SLE |
| FAS | FAS | Fas cell surface death | Pro-Apoptosis | 355 | — |
| | | receptor | | | |
| FASLG | FASLG | Fas ligand | Pro-Apoptosis | 356 | — |
| FAT4 | FAT4 | FAT atypical cadherin 4 | General-Cell-Surface | 79633 | — |
| FBN1 | FBN1 | fibrillin 1 | Integrin-Pathway | 2200 | — |
| FCGR1A | FCGR1A | Fc fragment of IgG receptor Ia | Immune-Cell-Surface | 2209 | — |
| FCGR3A | FCGR3A | Fc fragment of IgG receptor | Immune-Cell-Surface | 2214 | — |
| | | IIIa | | | |
| FCGR3B | FCGR3B | Fc fragment of IgG receptor | Immune-Cell-Surface | 2215 | — |
| | | IIIb | | | |
| FCN3 | FCN3 | ficolin 3 | Secreted-and-ECM | 8547 | — |
| FERMT3 | FERMT3 | fermitin family member 3 | Cytoskeleton | 83706 | — |
| FOXN1 | | | | 8456 | — |
| FOXP3 | FOXP3 | forkhead box P3 | Immune-Signaling | 50943 | — |
| FPR1 | FPR1 | formyl peptide receptor 1 | Immune-Cell-Surface | 2357 | — |
| G6PC | G6PC | glucose-6-phosphatase | Glycolysis- | 2538 | — |
| | | catalytic subunit | Gluconeogenesis- | | |
| | | | and-Pentose-Phosphate- | | |
| | | | Pathways | | |
| G6PC3 | G6PC3 | glucose-6-phosphatase | Glycolysis- | 92579 | — |
| | | catalytic subunit 3 | Gluconeogenesis- | | |
| | | | and-Pentose-Phosphate- | | |
| | | | Pathways | | |
| G6PD | G6PD | glucose-6-phosphate | Glycolysis- | 2539 | — |
| | | dehydrogenase | Gluconeogenesis- | | |
| | | | and-Pentose-Phosphate- | | |
| | | | Pathways | | |
| GATA2 | GATA2 | GATA binding protein 2 | Transcription-Factors | 2624 | — |
| GFI1 | GFI1 | growth factor independent | Transcription-Factors | 2672 | — |
| | | 1 transcriptional repressor | | | |
| GIF | | | | 2694 | High IL10 production |
| | | | | | associated with |
| | | | | | rheumatoid arthritis |
| | | | | | and SLE |
| GINS1 | GINS1 | GINS complex subunit 1 | Pro-Proliferation | 9837 | — |
| GPI | GPI | glucose-6-phosphate | Glycolysis- | 2821 | — |
| | | isomerase | Gluconeogenesis- | | |
| | | | and-Pentose-Phosphate- | | |
| | | | Pathways | | |
| HAX1 | HAX1 | HCLS1 associated protein X-1 | Endoplasmic-Reticulum | 10456 | — |
| HELLS | HELLS | helicase, lymphoid specific | Chromatin-Remodeling | 3070 | — |
| HEXB | HEXB | hexosaminidase subunit beta | Lysosome | 3074 | |
| HEXIM1 | HEXIM1 | hexamethylene | Transcription-Factors | 10614 | = |
| | | bisacetamide inducible 1 | | | |
| HMOX1 | HMOX1 | heme oxygenase 1 | Endoplasmic-Reticulum | 3162 | — |
| HOIP1 | | | | 55072 | — |
| HYOU1 | HYOU1 | hypoxia up-regulated 1 | Endoplasmic-Reticulum | 10525 | — |
| ICOS | ICOS | inducible T-cell costimulator | Immune-Cell-Surface | 29851 | |
| IFIH1 | IFIH1 | interferon induced with | Pattern-Recognition- | 64135 | — |
| | | helicase C domain 1 | Receptors | | |
| IFNAR2 | IFNAR2 | interferon alpha and beta | General-Cell-Surface | 3455 | — |
| | | receptor subunit 2 | | | |
| IFNG | IFNG | interferon gamma | Secreted-Immune | 3458 | — |
| IFNGR1 | IFNGR1 | interferon gamma receptor 1 | General-Cell-Surface | 3459 | — |
| IFNGR2 | IFNGR2 | interferon gamma receptor 2 | General-Cell-Surface | 3460 | — |
| IGAD1 | | | | 10986 | — |
| IGHA1 | IGHA1 | immunoglobulin heavy constant | Immune-Cell-Surface | 3493 | Mutation |
| | | alpha 1 | | | found in SLE |
| IGHA2 | IGHA2 | immunoglobulin heavy constant | Immune-Cell-Surface | 3494 | — |
| | | alpha 2 (A2m marker) | | | |
| IGHE | IGHE | immunoglobulin heavy constant | Immune-Cell-Surface | 3497 | — |
| | | epsilon | | | |
| IGHG1 | IGHG1 | immunoglobulin heavy constant | Immune-Cell-Surface | 3500 | pediatric SLE |
| | | gamma 1 (G1m marker) | | | |
| IGHG2 | | | | 3501 | — |
| IGHG3 | IGHG3 | immunoglobulin heavy constant | Immune-Cell-Surface | 3502 | — |
| | | gamma 3 (G3m marker) | | | |
| IGHG4 | | | | 3503 | |
| IGHM | IGHM | immunoglobulin heavy constant | Immune-Cell-Surface | 3507 | |
| | | mu | | | |
| IGKC | IGKC | immunoglobulin kappa constant | Immune-Cell-Surface | 3514 | — |
| IGLL1 | IGLL1 | immunoglobulin lambda | Immune-Cell-Surface | 3543 | — |
| | | like polypeptide 1 | | | |
| IKBKB | IKBKB | inhibitor of nuclear factor | Intracellular-Signaling | 3551 | — |
| | | kappa B kinase subunit beta | | | |
| IKBKG | IKBKG | inhibitor of nuclear factor | Intracellular-Signaling | 8517 | — |
| | | kappa B kinase subunit gamma | | | |
| IKZF1 | IKZF1 | IKAROS family zinc finger 1 | Immune-Signaling | 10320 | Causal mutation |
| | | | | | in SLE |
| IL10 | IL10 | interleukin 10 | Secreted-Immune | 3586 | |
| IL10RA | IL10RA | interleukin 10 receptor | Immune-Cell-Surface | 3587 | — |
| | | subunit alpha | | | |
| IL10RB | IL10RB | interleukin 10 receptor | Immune-Cell-Surface | 3588 | — |
| | | subunit beta | | | |
| IL12B | IL12B | interleukin 12B | Secreted-Immune | 3593 | — |
| IL12RB1 | IL12RB1 | interleukin 12 receptor | Immune-Cell-Surface | 3594 | novel treatment of |
| | | subunit beta 1 | | | lupus nephritis |
| | | | | | using CEP-33779 |
| | | | | | (orally active, |
| | | | | | selective inhibitor |
| | | | | | of JAK2; CP-690,550 |
| | | | | | treatment for LN and |
| | | | | | Jak-stat pathway |
| IL12RB2 | IL12RB2 | interleukin 12 receptor | Immune-Cell-Surface | 3595 | — |
| | | subunit beta 2 | | | |
| IL17A | IL17A | interleukin 17A | Secreted-Immune | 3605 | — |
| IL17F | IL17F | interleukin 17F | Secreted-Immune | 112744 | — |
| IL17RA | IL17RA | interleukin 17 receptor A | Immune-Cell-Surface | 23765 | — |
| IL17RC | IL17RC | interleukin 17 receptor C | Immune-Cell-Surface | 84818 | — |
| IL18 | IL18 | interleukin 18 | Secreted-Immune | 3606 | — |
| IL1RN | IL1RN | interleukin 1 receptor | Secreted-Immune | 3557 | |
| | | antagonist | | | |
| IL21 | IL21 | interleukin 21 | Secreted-Immune | 59067 | — |
| IL21R | IL21R | interleukin 21 receptor | Immune-Cell-Surface | 50615 | — |
| IL2RA | IL2RA | interleukin 2 receptor | Immune-Cell-Surface | 3559 | — |
| | | subunit alpha | | | |
| IL2RG | IL2RG | interleukin 2 receptor | Immune-Cell-Surface | 3561 | — |
| | | subunit gamma | | | |
| IL36RN | IL36RN | interleukin 36 receptor | Secreted-Immune | 26525 | — |
| | | antagonist | | | |
| IL6 | IL6 | interleukin 6 | Secreted-Immune | 3569 | — |
| IL6ST | IL6ST | interleukin 6 signal | Immune-Cell-Surface | 3572 | — |
| | | transducer | | | |
| IL7R | IL7R | interleukin 7 receptor | Immune-Cell-Surface | 3575 | — |
| INO80 | INO80 | INO80 complex subunit | Chromatin-Remodeling | 54617 | — |
| IRAK1 | IRAK1 | interleukin 1 receptor | Pattern-Recognition- | 3654 | — |
| | | associated kinase 1 | Receptors | | |
| IRAK4 | IRAK4 | interleukin 1 receptor | Pattern-Recognition- | 51135 | |
| | | associated kinase 4 | Receptors | | |
| IRF2BP2 | IRF2BP2 | interferon regulatory | Pattern-Recognition- | 359948 | — |
| | | factor 2 binding protein 2 | Receptors | | |
| IRF3 | IRF3 | interferon regulatory factor 3 | Pattern-Recognition- | 3661 | — |
| | | | Receptors | | |
| IRF4 | IRF4 | interferon regulatory factor 4 | Pattern-Recognition- | 3662 | |
| | | | Receptors | | |
| IRF7 | IRF7 | interferon regulatory factor 7 | Pattern-Recognition- | 3665 | — |
| | | | Receptors | | |
| IRF8 | IRF8 | interferon regulatory factor 8 | Pattern-Recognition- | 3394 | — |
| | | | Receptors | | |
| ISG15 | ISG15 | ISG15 ubiquitin-like modifier | Pattern-Recognition- | 9636 | — |
| | | | Receptors | | |
| ITCH | ITCH | itchy E3 ubiquitin protein | Ubiquitylation- | 83737 | — |
| | | ligase | and-Sumoylation | | |
| ITGAM | ITGAM | integrin subunit alpha M | Integrin-Pathway | 3684 | — |
| ITGAX | ITGAX | integrin subunit alpha X | Integrin-Pathway | 3687 | — |
| ITGB2 | ITGB2 | integrin subunit beta 2 | Integrin-Pathway | 3689 | — |
| ITK | ITK | IL2 inducible T-cell kinase | Immune-Signaling | 3702 | — |
| JAGN1 | JAGN1 | jagunal homolog 1 | Endoplasmic-Reticulum | 84522 | — |
| JAK1 | JAK1 | Janus kinase 1 | Intracellular-Signaling | 3716 | |
| JAK3 | JAK3 | Janus kinase 3 | Intracellular-Signaling | 3718 | — |
| KDM6A | KDM6A | lysine demethylase 6A | Chromatin-Remodeling | 7403 | — |
| KMT2A | KMT2A | lysine methyltransferase 2A | Chromatin-Remodeling | 4297 | — |
| KMT2D | KMT2D | lysine methyltransferase 2D | Chromatin-Remodeling | 8085 | — |
| KRAS | KRAS | KRAS proto-oncogene, GTPase | Intracellular-Signaling | 3845 | — |
| LAMTOR2 | LAMTOR2 | late endosomal/lysosomal | Endosome-and-Vesicles | 28956 | — |
| | | adaptor, MAPK and MTOR | | | |
| | | activator 2 | | | |
| LAT | LAT | linker for activation of | Immune-Signaling | 27040 | — |
| | | T-cells | | | |
| LCK | LCK | LCK proto-oncogene, Src | Immune-Signaling | 3932 | — |
| | | family tyrosine kinase | | | |
| LIG1 | LIG1 | DNA ligase 1 | DNA-Repair | 3978 | — |
| LIG4 | LIG4 | DNA ligase 4 | DNA-Repair | 3981 | — |
| LIPA | LIPA | lipase A, lysosomal acid | Lysosome | 3988 | — |
| | | type | | | |
| LPIN2 | LPIN2 | lipin 2 | Cytoplasm-and-Biochemistry | 9663 | — |
| LRBA | LRBA | LPS responsive beige-like | Golgi | 987 | — |
| | | anchor protein | | | |
| LRRC8A | LRRC8A | leucine rich repeat containing | Transporters | 56262 | SLE, Vitiligo- |
| | | 8 family member A | | | related 1 |
| LYST | LYST | lysosomal trafficking | Endosome-and-Vesicles | 1130 | — |
| | | regulator | | | |
| MAGT1 | MAGT1 | magnesium transporter 1 | Transporters | 84061 | — |
| MALT1 | MALT1 | MALT1 paracaspase | Immune-Signaling | 10892 | — |
| MAP3K14 | MAP3K14 | mitogen-activated protein | Intracellular-Signaling | 9020 | — |
| | | kinase kinase kinase 14 | | | |
| MASP1 | MASP1 | mannan binding lectin | Secreted-Immune | 5648 | — |
| | | serine peptidase 1 | | | |
| MASP2 | MASP2 | mannan binding lectin | Golgi | 10747 | — |
| | | serine peptidase 2 | | | |
| MBL2 | | | | 4153 | — |
| MBTPS2 | MBTPS2 | membrane bound transcription | Unfolded-Protein- | 51360 | |
| | | factor peptidase, site 2 | and-Stress | | |
| MCM4 | MCM4 | minichromosome maintenance | Pro-Proliferation | 4173 | — |
| | | complex component 4 | | | |
| MEFV | MEFV | MEFV, pyrin innate immunity | Pattern-Recognition- | 4210 | — |
| | | regulator | Receptors | | |
| MKL1 | MKL1 | megakaryoblastic leukemia | Transcription-Factors | 57591 | — |
| | | (translocation) 1 | | | |
| MLPH | MLPH | melanophilin | Melanosome | 79083 | — |
| MOGS | | | | 7841 | — |
| MPO | MPO | myeloperoxidase | Secreted-Immune | 4353 | — |
| MRE11A | | | | 4361 | |
| MS4A1 | MS4A1 | membrane spanning 4-domains A1 | Immune-Cell-Surface | 931 | — |
| MSH6 | MSH6 | mutS homolog 6 | DNA-Repair | 2956 | — |
| MSN | MSN | moesin | Cytoskeleton | 4478 | systemic |
| | | | | | manifestations |
| MTHFD1 | MTHFD1 | methylenetetrahydrofolate | Cytoplasm-and- | 4522 | — |
| | | dehydrogenase, cyclohydrolase | Biochemistry | | |
| | | and formyltetrahydrofolate | | | |
| | | synthetase 1 | | | |
| MVK | MVK | mevalonate kinase | Cytoplasm-and- | 4598 | — |
| | | | Biochemistry | | |
| MX2 | MX2 | MX dynamin like GTPase 2 | Interferon- | 4600 | — |
| | | | Stimulated-Genes | | |
| MYB | MYB | MYB proto-oncogene, | Transcription-Factors | 4602 | — |
| | | transcription factor | | | |
| MYD88 | MYD88 | myeloid differentiation | Pattern-Recognition- | 4615 | — |
| | | primary response 88 | Receptors | | |
| MYH9 | MYH9 | myosin heavy chain 9 | Cytoskeleton | 4627 | — |
| MYO5A | MYO5A | myosin VA | Cytoskeleton | 4644 | |
| MYSM1 | MYSM1 | Myb like, SWIRM and | Chromatin-Remodeling | 114803 | — |
| | | MPN domains 1 | | | |
| NBAS | NBAS | neuroblastoma amplified | Golgi | 51594 | — |
| | | sequence | | | |
| NBN | NBN | nibrin | DNA-Repair | 4683 | |
| NCF1 | NCF1 | neutrophil cytosolic factor 1 | Reactive-Oxygen- | 653361 | — |
| | | | Species-Protection | | |
| NCF2 | NCF2 | neutrophil cytosolic factor 2 | Reactive-Oxygen- | 4688 | — |
| | | | Species-Protection | | |
| NCF4 | NCF4 | neutrophil cytosolic factor 4 | Reactive-Oxygen- | 4689 | — |
| | | | Species-Protection | | |
| NCSTN | NCSTN | nicastrin | General-Cell-Surface | 23385 | Lupus association |
| NEAT1 | NEAT1 | nuclear paraspeckle assembly | Unknown | 283131 | — |
| | | transcript 1 | | | |
| | | (non-protein coding) | | | |
| NEIL3 | | | | 55247 | — |
| NFAT5 | NFAT5 | nuclear factor of activated | Intracellular-Signaling | 10725 | — |
| | | T-cells 5 | | | |
| NFKB1 | NFKB1 | nuclear factor kappa B | Intracellular-Signaling | 4790 | — |
| | | subunit 1 | | | |
| NFKB2 | NFKB2 | nuclear factor kappa B | Intracellular-Signaling | 4791 | — |
| | | subunit 2 | | | |
| NFKBIA | NFKBIA | NFKB inhibitor alpha | Intracellular-Signaling | 4792 | — |
| NHEJ1 | NHEJ1 | non-homologous end joining | DNA-Repair | 79840 | — |
| | | factor 1 | | | |
| NHP2 | NHP2 | NHP2 ribonucleoprotein | mRNA-Translation | 55651 | — |
| NLRC4 | NLRC4 | NLR family CARD domain | Pattern-Recognition- | 58484 | — |
| | | containing 4 | Receptors | | |
| NLRP1 | NLRP1 | NLR family pyrin domain | Pattern-Recognition- | 22861 | — |
| | | containing 1 | Receptors | | |
| NLRP12 | NLRP12 | NLR family pyrin domain | Pattern-Recognition- | 91662 | — |
| | | containing 12 | Receptors | | |
| NLRP3 | NLRP3 | NLR family pyrin domain | Pattern-Recognition- | 114548 | |
| | | containing 3 | Receptors | | |
| NOD2 | NOD2 | nucleotide binding | Pattern-Recognition- | 64127 | — |
| | | oligomerization domain | Receptors | | |
| | | containing 2 | | | |
| NOP10 | NOP10 | NOP10 ribonucleoprotein | Nucleus-and-Nucleolus | 55505 | — |
| NRAS | NRAS | NRAS proto-oncogene, GTPase | Intracellular-Signaling | 4893 | — |
| NSMCE3 | NSMCE3 | NSE3 homolog, SMC5- | DNA-Repair | 56160 | — |
| | | SMC6 complex component | | | |
| ORAI1 | ORAI1 | ORAI calcium release-activated | Transporters | 84876 | — |
| | | calcium modulator 1 | | | |
| OSTM1 | OSTM1 | osteopetrosis associated | Unknown | 28962 | Mutation involved |
| | | transmembrane protein 1 | | | in lupus |
| | | | | | pathogenesis |
| OTULIN | | | | 90268 | — |
| PARN | PARN | poly(A)-specific ribonuclease | mRNA-Translation | 5073 | — |
| PAX1 | PAX1 | paired box 1 | Transcription-Factors | 5075 | If a defieciency, |
| | | | | | lupus like symptoms |
| PCNA | PCNA | proliferating cell nuclear | Pro-Proliferation | 5111 | — |
| | | antigen | | | |
| PEPD | PEPD | peptidase D | Secreted-and-ECM | 5184 | — |
| PEX1 | PEX1 | peroxisomal biogenesis factor 1 | Peroxisomes | 5189 | |
| PGM3 | PGM3 | phosphoglucomutase 3 | Cytoplasm-and- | 5238 | — |
| | | | Biochemistry | | |
| PIEZO1 | PIEZO1 | piezo type mechanosensitive | Transporters | 9780 | — |
| | | ion channel component 1 | | | |
| PIGA | PIGA | phosphatidylinositol glycan | Endoplasmic-Reticulum | 5277 | — |
| | | anchor biosynthesis class A | | | |
| PIK3CD | PIK3CD | phosphatidylinositol-4,5- | Cytoplasm-and- | 5293 | — |
| | | bisphosphate 3-kinase | Biochemistry | | |
| | | catalytic subunit delta | | | |
| PIK3R1 | PIK3R1 | phosphoinositide-3- | Cytoplasm-and- | 5295 | — |
| | | kinase regulatory subunit 1 | Biochemistry | | |
| PLCG2 | PLCG2 | phospholipase C gamma 2 | Integrin-Pathway | 5336 | — |
| PLEKHM1 | PLEKHM1 | pleckstrin homology and | Endosome-and-Vesicles | 9842 | — |
| | | RUN domain containing M1 | | | |
| PLXNA1 | PLXNA1 | plexin A1 | General-Cell-Surface | 5361 | Mutation resulted |
| | | | | | In one patient |
| | | | | | developing SLE |
| PMS2 | PMS2 | PMS1 homolog 2, mismatch | DNA-Repair | 5395 | — |
| | | repair system component | | | |
| PNP | PNP | purine nucleoside | Cytoplasm-and- | 4860 | — |
| | | phosphorylase | Biochemistry | | |
| POLA1 | POLA1 | DNA polymerase alpha 1, | Pro-Proliferation | 5422 | — |
| | | catalytic subunit | | | |
| POLE1 | POLE | DNA polymerase epsilon, | DNA-Repair | 5426 | — |
| | | catalytic subunit | | | |
| POLE2 | POLE2 | DNA polymerase epsilon | DNA-Repair | 5427 | — |
| | | 2, accessory subunit | | | |
| PRF1 | PRF1 | perforin 1 | Immune-Signaling | 5551 | — |
| PRKCD | PRKCD | protein kinase C delta | Intracellular-Signaling | 5580 | — |
| PRKDC | PRKDC | protein kinase, DNA-activated, | DNA-Repair | 5591 | |
| | | catalytic polypeptide | | | |
| PSEN1 | PSEN1 | presenilin 1 | Golgi | 5663 | — |
| PSENEN | PSENEN | presenilin enhancer | Golgi | 55851 | — |
| | | gamma-secretase subunit | | | |
| PSMB8 | PSMB8 | proteasome subunit beta 8 | Proteasome | 5696 | — |
| PSTPIP1 | PSTPIP1 | proline-serine-threonine | Immune-Signaling | 9051 | — |
| | | phosphatase interacting | | | |
| | | protein 1 | | | |
| PTEN | PTEN | phosphatase and tensin homolog | Intracellular-Signaling | 5728 | — |
| PTPN6 | PTPN6 | protein tyrosine phosphatase, | Immune-Signaling | 5777 | — |
| | | non-receptor type 6 | | | |
| PTPRC | PTPRC | protein tyrosine phosphatase, | Immune-Cell-Surface | 5788 | — |
| | | receptor type C | | | |
| RAB27A | RAB27A | RAB27A, member RAS oncogene | Endosome-and-Vesicles | 5873 | — |
| | | family | | | |
| RAC1 | RAC1 | ras-related C3 botulinum | Integrin-Pathway | 5879 | — |
| | | toxin substrate 1 (rho family, | | | |
| | | small GTP binding protein Rac1) | | | |
| RAC2 | RAC2 | ras-related C3 botulinum | Integrin-Pathway | 5880 | — |
| | | toxin substrate 2 (rho family, | | | |
| | | small GTP binding protein Rac2) | | | |
| RAD52 | RAD52 | RAD52 homolog, DNA repair | DNA-Repair | 5893 | |
| | | protein | | | |
| RAG1 | RAG1 | recombination activating 1 | DNA-Repair | 5896 | — |
| RAG2 | RAG2 | recombination activating 2 | DNA-Repair | 5897 | — |
| RANBP2 | RANBP2 | RAN binding protein 2 | Nucleus-and-Nucleolus | 5903 | |
| RASGRP1 | RASGRP1 | RAS guanyl releasing protein 1 | RAS-Superfamily | 10125 | |
| RASGRP2 | RASGRP2 | RAS guanyl releasing protein 2 | RAS-Superfamily | 10235 | — |
| RBCK1 | RBCK1 | RANBP2-type and C3HC4-type | Ubiquitylation- | 10616 | — |
| | | zinc finger containing 1 | and-Sumoylation | | |
| RECQL4 | RECQL4 | RecQ like helicase 4 | Pro-Proliferation | 9401 | — |
| RELB | RELB | RELB proto-oncogene, NF-kB | Intracellular-Signaling | 5971 | — |
| | | subunit | | | |
| RET | RET | ret proto-oncogene | Endocytosis | 5979 | — |
| RFX5 | RFX5 | regulatory factor X5 | MHC-Class-TWO | 5993 | — |
| RFXANK | RFXANK | regulatory factor X associated | MHC-Class-TWO | 8625 | — |
| | | ankyrin containing protein | | | |
| RFXAP | RFXAP | regulatory factor X associated | Transcription-Factors | 5994 | — |
| | | protein | | | |
| RHOH | RHOH | ras homolog family member H | RAS-Superfamily | 399 | — |
| RMRP | RMRP | RNA component of mitochondrial | Mitochondria-General | 6023 | — |
| | | RNA processing endoribonuclease | | | |
| RNASEH2A | RNASEH2A | ribonuclease H2 subunit A | Pro-Proliferation | 10535 | Gene incodes BAFF |
| | | | | | which is increased |
| | | | | | in patients with SLE |
| RNASEH2B | RNASEH2B | ribonuclease H2 subunit B | Pro-Proliferation | 79621 | — |
| RNASEH2C | RNASEH2C | ribonuclease H2 subunit C | Pro-Proliferation | 84153 | OX40 deficiency |
| | | | | | correlated in SLE |
| RNASEL | RNASEL | ribonuclease L | mRNA-Translation | 6041 | — |
| RNF168 | RNF168 | ring finger protein 168 | DNA-Repair | 165918 | — |
| RNF31 | RNF31 | ring finger protein 31 | Intracellular-Signaling | 55072 | — |
| RNU4ATAC | | | | 100151683 | — |
| ROR2 | ROR2 | receptor tyrosine kinase | WNT-Signaling | 4920 | increased |
| | | like orphan receptor 2 | | | susceptibility |
| | | | | | to SLE |
| RORC | RORC | RAR related orphan receptor C | Immune-Signaling | 6097 | |
| RPL35A | RPL35A | ribosomal protein L35a | mRNA-Translation | 6165 | |
| RPL5 | RPL5 | ribosomal protein L5 | mRNA-Translation | 6125 | |
| RPSA | RPSA | ribosomal protein SA | mRNA-Translation | 3921 | |
| RTEL1 | RTEL1 | regulator of telomere | DNA-Repair | 51750 | Initially identified |
| | | elongation helicase 1 | | | as the cellular receptor |
| | | | | | for HIV, but expression |
| | | | | | of CD4 alone insufficient |
| | | | | | to confersuscpetibility |
| | | | | | to HIV |
| RUNX1 | RUNX1 | runt related transcription | Immune-Signaling | 861 | Susceptibility |
| | | factor 1 | | | to lupus nephritis |
| SAMD9 | SAMD9 | sterile alpha motif domain | Interferon- | 54809 | Susceptibility |
| | | containing 9 | Stimulated-Genes | | to SLE |
| SAMD9L | SAMD9L | sterile alpha motif | Interferon- | 219285 | |
| | | domain containing 9 like | Stimulated-Genes | | |
| SAMHD1 | SAMHD1 | SAM and HD domain containing | Cytoplasm-and- | 25939 | |
| | | deoxynucleoside triphosphate | Biochemistry | | |
| | | triphosphohydrolase 1 | | | |
| SBDS | SBDS | SBDS, ribosome maturation | mRNA-Translation | 51119 | |
| | | factor | | | |
| SEC61A1 | SEC61A1 | Sec61 translocon alpha 1 | Endoplasmic-Reticulum | 29927 | |
| | | subunit | | | |
| SEMA3E | | | | 9723 | |
| SERPING1 | SERPING1 | serpin family G member 1 | Secreted-and-ECM | 710 | |
| SH2D1A | SH2D1A | SH2 domain containing 1A | Immune-Signaling | 4068 | |
| SH3BP2 | SH3BP2 | SH3 domain binding protein 2 | Unknown | 6452 | |
| SKIV2L | SKIV2L | Ski2 like RNA helicase | Nucleus-and-Nucleolus | 6499 | |
| SLC11A1 | SLC11A1 | solute carrier family 11 | Transporters | 6556 | |
| | | member 1 | | | |
| SLC29A3 | SLC29A3 | solute carrier family 29 | Lysosome | 55315 | |
| | | member 3 | | | |
| SLC35C1 | SLC35C1 | solute carrier family 35 | Golgi | 55343 | |
| | | member C1 | | | |
| SLC37A4 | SLC37A4 | solute carrier family 37 | Glycolysis- | 2542 | |
| | | member 4 | Gluconeogenesis- | | |
| | | | and-Pentose-Phosphate- | | |
| | | | Pathways | | |
| SLC46A1 | SLC46A1 | solute carrier family 46 | Transporters | 113235 | |
| | | member 1 | | | |
| SMARCAL1 | SMARCAL1 | SWI/SNF related, matrix | Chromatin-Remodeling | 50485 | |
| | | associated, actin dependent | | | |
| | | regulator of chromatin, | | | |
| | | subfamily a like 1 | | | |
| SMARCD2 | SMARCD2 | SWI/SNF related, matrix | Chromatin-Remodeling | 6603 | |
| | | associated, actin dependent | | | |
| | | regulator of chromatin, | | | |
| | | subfamily d, member 2 | | | |
| SNX10 | SNX10 | sorting nexin 10 | Endosome-and-Vesicles | 29887 | |
| SP110 | SP110 | SP110 nuclear body protein | Interferon- | 3431 | |
| | | | Stimulated-Genes | | |
| SPINK5 | | | | 11005 | |
| SRP54 | SRP54 | signal recognition particle 54 | Endoplasmic-Reticulum | 6729 | |
| STAT1 | STAT1 | signal transducer and | Intracellular-Signaling | 6772 | |
| | | activator of transcription 1 | | | |
| STAT2 | STAT2 | signal transducer and | Intracellular-Signaling | 6773 | |
| | | activator of transcription 2 | | | |
| STAT3 | STAT3 | signal transducer and | Intracellular-Signaling | 6774 | |
| | | activator of transcription 3 | | | |
| STAT5B | STAT5B | signal transducer and | Intracellular-Signaling | 6777 | |
| | | activator of transcription 5B | | | |
| STIM1 | STIM1 | stromal interaction molecule 1 | Endoplasmic-Reticulum | 6786 | |
| STK4 | STK4 | serine/threonine kinase 4 | Immune-Signaling | 6789 | |
| STN1 | STN1 | STN1, CST complex subunit | Nucleus-and-Nucleolus | 79991 | |
| STX11 | STX11 | syntaxin 11 | Endosome-and-Vesicles | 8676 | |
| STXBP2 | STXBP2 | syntaxin binding protein 2 | Endosome-and-Vesicles | 6813 | |
| TADA2A | TADA2A | transcriptional adaptor 2A | General-Transcription | 6871 | |
| TAOK2 | TAOK2 | TAO kinase 2 | Intracellular-Signaling | 9344 | |
| TAP1 | TAP1 | transporter 1, ATP binding | MHC-Class-ONE | 6890 | |
| | | cassette subfamily B member | | | |
| TAP2 | TAP2 | transporter 2, ATP binding | MHC-Class-ONE | 6891 | |
| | | cassette subfamily B member | | | |
| TAPBP | TAPBP | TAP binding protein | MHC-Class-ONE | 6892 | |
| TARBP2 | TARBP2 | TARBP2, RISC loading complex | MicroRNA-Processing | 6895 | |
| | | RNA binding subunit | | | |
| TAZ | TAZ | tafazzin | Mitochondria-General | 6901 | |
| TBK1 | TBK1 | TANK binding kinase 1 | Pattern-Recognition- | 29110 | |
| | | | Receptors | | |
| TBX1 | TBX1 | T-box 1 | Transcription-Factors | 6899 | |
| TCF3 | TCF3 | transcription factor 3 | Transcription-Factors | 6929 | |
| TCIRG1 | TCIRG1 | T-cell immune regulator 1, | Lysosome | 10312 | |
| | | ATPase H+ transporting | | | |
| | | V0 subunit a3 | | | |
| TCN2 | TCN2 | transcobalamin 2 | Transporters | 6948 | |
| TECR | TECR | trans-2,3-enoyl-CoA reductase | Endoplasmic-Reticulum | 9524 | |
| TERC | TERC | telomerase RNA component | Nucleus-and-Nucleolus | 7012 | |
| TERT | TERT | telomerase reverse | Pro-Proliferation | 7015 | |
| | | transcriptase | | | |
| TFRC | TFRC | transferrin receptor | Endosome-and-Vesicles | 7037 | |
| TGFBR1 | TGFBR1 | transforming growth factor | General-Cell-Surface | 7046 | |
| | | beta receptor 1 | | | |
| TGFBR2 | TGFBR2 | transforming growth factor | General-Cell-Surface | 7048 | |
| | | beta receptor 2 | | | |
| THBD | THBD | thrombomodulin | Immune-Cell-Surface | 7056 | |
| TICAM1 | TICAM1 | toll like receptor adaptor | Pattern-Recognition- | 148022 | |
| | | molecule 1 | Receptors | | |
| TINF2 | TINF2 | TERF1 interacting nuclear | Nucleus-and-Nucleolus | 26277 | |
| | | factor 2 | | | |
| TIRAP | TIRAP | TIR domain containing adaptor | Pattern-Recognition- | 114609 | |
| | | protein | Receptors | | |
| TLR3 | TLR3 | toll like receptor 3 | Pattern-Recognition- | 7098 | |
| | | | Receptors | | |
| TMC6 | TMC6 | transmembrane channel like 6 | Unknown | 11322 | |
| TMC8 | TMC8 | transmembrane channel like 8 | Transporters | 147138 | |
| TMEM173 | TMEM173 | transmembrane protein 173 | Pattern-Recognition- | 340061 | |
| | | | Receptors | | |
| TNFAIP3 | TNFAIP3 | TNF alpha induced protein 3 | Pattern-Recognition- | 7128 | |
| | | | Receptors | | |
| TNFRSF11A | TNFRSF11A | TNF receptor superfamily | Immune-Cell-Surface | 8792 | |
| | | member 11a | | | |
| TNFRSF13B | TNFRSF13B | TNF receptor superfamily | Immune-Cell-Surface | 23495 | |
| | | member 13B | | | |
| TNFRSF13C | TNFRSF13C | TNF receptor superfamily | Immune-Cell-Surface | 115650 | |
| | | member 13C | | | |
| TNFRSF1A | TNFRSF1A | TNF receptor superfamily | Pro-Apoptosis | 7132 | |
| | | member 1A | | | |
| TNFRSF4 | TNFRSF4 | TNF receptor superfamily | Immune-Cell-Surface | 7293 | |
| | | member 4 | | | |
| TNFSF10 | TNFSF10 | TNF superfamily member 10 | Pro-Apoptosis | 8743 | |
| TNFSF11 | TNFSF11 | TNF superfamily member 11 | Secreted-Immune | 8600 | |
| TNFSF12 | TNFSF12 | TNF superfamily member 12 | Pro-Apoptosis | 8742 | |
| TPP1 | TPP1 | tripeptidyl peptidase 1 | Lysosome | 1200 | |
| TPP2 | TPP2 | tripeptidyl peptidase 2 | Ubiquitylation- | 7174 | |
| | | | and-Sumoylation | | |
| TRAC | TRAC | T-cell receptor alpha constant | Immune-Cell-Surface | 28755 | |
| TRAF3 | TRAF3 | TNF receptor associated | Pattern-Recognition- | 7187 | |
| | | factor 3 | Receptors | | |
| TRAF3IP2 | TRAF3IP2 | TRAF3 interacting protein 2 | Intracellular-Signaling | 10758 | |
| TREX1 | TREX1 | three prime repair | Pattern-Recognition- | 11277 | |
| | | exonuclease 1 | Receptors | | |
| TRIM25 | TRIM25 | tripartite motif containing 25 | Pattern-Recognition- | 7706 | |
| | | | Receptors | | |
| TRNT1 | TRNT1 | tRNA nucleotidyl transferase 1 | Mitochondria- | 51095 | |
| | | | DNA-to-RNA | | |
| TTC37 | TTC37 | tetratricopeptide repeat | mRNA-Translation | 9652 | |
| | | domain 37 | | | |
| TTC7A | TTC7A | tetratricopeptide repeat | General-Cell-Surface | 57217 | |
| | | domain 7A | | | |
| TYK2 | TYK2 | tyrosine kinase 2 | Intracellular-Signaling | 7297 | |
| UNC119 | UNC119 | unc-119 lipid binding chaperone | Unknown | 9094 | |
| UNC13D | UNC13D | unc-13 homolog D | Endosome-and-Vesicles | 201294 | |
| UNC93B1 | UNC93B1 | unc-93 homolog B1 (C. elegans) | Pattern-Recognition- | 81622 | |
| | | | Receptors | | |
| UNG | UNG | uracil DNA glycosylase | Mitochondria-General | 7374 | |
| USB1 | USB1 | U6 snRNA biogenesis | Nucleus-and-Nucleolus | 79650 | |
| | | phosphodiesterase 1 | | | |
| USP18 | USP18 | ubiquitin specific peptidase 18 | Ubiquitylation- | 11274 | |
| | | | and-Sumoylation | | |
| VAV1 | VAV1 | vav guanine nucleotide exchange | Immune-Signaling | 7409 | |
| | | factor 1 | | | |
| VPREB1 | VPREB1 | V-set pre-B cell surrogate | Immune-Cell-Surface | 7441 | |
| | | light chain 1 | | | |
| VPS13B | VPS13B | vacuolar protein sorting 13 | Golgi | 157680 | |
| | | homolog B | | | |
| VPS45 | VPS45 | vacuolar protein sorting 45 | Endocytosis | 11311 | |
| | | homolog | | | |
| WAS | WAS | Wiskott-Aldrich syndrome | Cytoskeleton | 7454 | |
| WDR1 | WDR1 | WD repeat domain 1 | Cytoskeleton | 9948 | |
| WIPF1 | WIPF1 | WAS/WASL interacting protein | Endocytosis | 7456 | |
| | | family member 1 | | | |
| WNT10A | WNT10A | Wnt family member 10A | WNT-Signaling | 80326 | |
| WRAP53 | WRAP53 | WD repeat containing antisense | Nucleus-and-Nucleolus | 55135 | |
| | | to TP53 | | | |
| XIAP | XIAP | X-linked inhibitor of apoptosis | Anti-Apoptosis | 331 | |
| XRCC4 | XRCC4 | X-ray repair cross | DNA-Repair | 7518 | |
| | | complementing 4 | | | |
| ZAP70 | ZAP70 | zeta chain of T-cell receptor | Immune-Signaling | 7535 | |
| | | associated protein kinase 70 | | | |
| ZBTB24 | ZBTB24 | zinc finger and BTB domain | Transcription-Factors | 9841 | |
| | | containing 24 | | | |
| CD4 | CD4 | CD4 Molecule | | 920 | |
| P14 | CDKN2A | Cyclin Dependent Kinase | Anti-Proliferation | 1029 | |
| | | Inhibitor 2A | | | |
| FCGR2A | FCGR2A | Fc Fragment of IgG Receptor Iia | Immune-Cell-Surface | 2212 | |
| FCGR2B | FCGR2B | Fc Fragment of IgG Receptor IIb | Immune-Cell-Surface | 2213 | |
| FCGRT | FCGRT | Fc Fragment of IgG Receptor | General-Cell-Surface | 2217 | |
| | | and Transporter | | | |
| REL | REL | REL Proto-Oncogene, NF-KB | Intracellular-Signaling | 5966 | |
| | | Subunit | | | |
| STAT5A | STAT5A | Signal Transducer and | Intracellular-Signaling | 6776 | |
| | | Activator of Transcription 5A | | | |
| RIPK1 | RIPK1 | Receptor Interacting | Anti-Apoptosis; | 8737 | |
| | | Serine/Threonine Kinase 1 | Intracellular-Signaling | | |
| ARHGEF1 | ARHGEF1 | Rho Guanine Nucleotide | RAS-Superfamily | 9138 | |
| | | Exchange Factor 1 | | | |
| IRF9 | IRF9 | Interferon Regulatory Factor | Pattern-Recognition- | 10379 | |
| | | 9 | Receptors | | |
| TWEAK | TNFSF25 | Tumor Necrosis Factor Receptor | Pro-Apoptosis | 51130 | |
| | | Superfamily, Member 12 | | | |
| CYBC1 | CYBC1 | Cytochrome B-245 Chaperone 1 | | 79415 | |
| SPPL2A | SPPL2A | Signal Peptide Peptidase | General-Cell-Surface | 84888 | |
| | | Like 2A | | | |
| ZNF341 | ZNF341 | Zinc Finger Protein 341 | Transcription-Factors | 84905 | |
| LILRA5 | LILRA5 | Leukocyte Immunoglobulin | Immune-Cell-Surface | 353514 | |
| | | Like Receptor A5 | | | |
| CAD14 | | | | | |
| TNRT1 | | | | | |

Tables 48A-48D show PID-associated genes (e.g., genes that are DE in PID) that overlap with E-genes, C-genes, P-genes, and T-genes, respectively. These E-genes, C-genes, P-genes, and T-genes contain single nucleotide polymorphisms (SNPs) that can be used as marker genes for PID and/or lupus conditions. Further, the E-genes, C-genes, P-genes, and T-genes can be used as marker genes in certain populations of subjects, depending on ancestry (e.g., African, European, or shared). For example, of the 759 E-genes, 27 are PID-associated genes (Table 48A). As another example, of the 22 C-genes, 6 are PID-associated genes (Table 48B). As another example, of the 520 P-genes, 30 are PID-associated genes (Table 48C). As another example, of the 627 T-genes, 19 are PID-associated genes (Table 48D).

TABLE 48A
PID-associated genes that overlap with E-genes

| Gene | |
|---|---|
| Symbol | Ancestry |
| AP3D1 | European |
| C1QB | European |
| CARD9 | European |
| CASP10 | European |
| CASP8 | European |
| CD40 | European |
| CFH | European |
| CFHR1 | European |
| CFHR3 | European |
| CFHR4 | European |
| COPA | European |
| CTLA4 | European |
| DNMT3B | European |
| IL12RB1 | European |
| IL12RB2 | European |
| INO80 | European |
| IRAK4 | European |
| ITGAX | African |
| LAT | African |
| NCF2 | Shared (African and European) |
| NCSTN | Shared (African and European) |
| NEAT1 | Shared (African and European) |
| PLEKHM1 | Shared (African and European) |
| SH3BP2 | Shared (African and European) |
| TRAF3IP2 | Shared (African and European) |
| UNC13D | Shared (African and European) |
| FCGRT | Shared (African and European) |

TABLE 48B
PID-associated genes that overlap with C-genes

| Gene | |
|---|---|
| Symbol | Ancestry |
| CR2 | European |
| FCGR2A | European |
| TNFAIP3 | European |
| TYK2 | European |
| IFIH1 | European |
| ITGAM | Shared (African and European) |

TABLE 48C
PID-associated genes that overlap with P-genes

| Gene | |
|---|---|
| Symbol | Ancestry |
| AICDA | European |
| AIRE | European |
| BACH2 | European |
| CCL22 | European |
| CR2 | European |
| CTLA4 | European |
| DOCK8 | European |
| FCGR2A | European |
| GFI1 | European |
| ICOS | European |
| IFIH1 | European |
| IFNG | European |
| IL10 | European |
| IL10RA | European |
| IL12RB1 | European |
| IL21 | European |
| IL2RA | European |
| IL7R | European |
| IRF7 | European |
| IRF8 | European |
| ITGAM | European |
| LYST | European |
| PTPRC | European |
| TNFAIP3 | European |
| TRAF3 | European |
| TYK2 | European |
| C5 | African |
| IKZF1 | African |
| FASLG | Shared (African and European) |
| IRF4 | Shared (African and European) |

TABLE 48D
PID-associated genes that overlap with T-genes

| Gene | |
|---|---|
| Symbol | Ancestry |
| CD3E | European |
| CD40 | European |
| CHD7 | European |
| FASLG | European |
| FCGR2A | European |
| IKZF1 | European |
| IL12RB1 | European |
| IL2RA | European |
| INO80 | European |
| IRF8 | European |
| ITCH | European |
| JAK3 | European |
| NCF2 | European |
| PNP | European |
| SLC37A4 | European |
| STAT1 | European |
| TNFAIP3 | European |
| TRIM25 | European |
| ZBTB24 | Shared (African and European) |

Table 49A shows a list of genes with a mouse model available for experimentation purposes. For example, these genes may be evaluated for their suitability as potential “knockout” genes. These genes and their associated pathways may be used as unique targets for novel therapies in SLE.

TABLE 49A
Genes with mouse model available for experimentation purposes

ACD
ACTB
ADA2
ADAR
AIRE
AP3B1
APOL1
ATM
ATP6V0A2
B2M
BCL10
BLM
C4BPA
C5
CARD14
CARD9
CASP10
CASP8
CCBE1
CCL2
CD46
CD79A
CFB
CFHR1
CFHR3
CFHR5
CFI
CFTR
CHD7
CIITA
CR2
CSF3R
CTLA4
CTSC
DKC1
DOCK8
ELANE
F12
FADD
FAS
FASLG
FBN1
FCGR3B
FOXP3
G6PC3
G6PD
GATA2
GINS1
GPI
HAX1
IFIH1
IFNG
IGHM
IKBKG
IL10
IL21R
IL6
ITGAM
ITGB2
JAK3
KRAS
MCM4
MSH6
MTHFD1
MYB
MYH9
NBN
NHP2
NLRP3
NRAS
PARN
PIEZO1
PIK3R1
PMS2
POLE1
PRF1
PTEN
RAC1
RAG1
RAG2
RET
RNASEH2A
RNASEH2B
RNASEH2C
RNASEL
RNU4ATAC
ROR2
RPL35A
RPL5
RPSA
RTEL1
RUNX1
SAMHD1
SBDS
SEC61A1
SLC46A1
STAT3
STAT5B
STIM1
STX11
STXBP2
TCIRG1
TERC
TERT
THBD
TINF2
TNFRSF11A
TRAF3IP2
TREX1
UNC119
VPS45
WAS
WRAP53
XIAP
XRCC4

In conclusion, of the 450 PID-associated genes in the database that were identified via literature mining, 125 genes were determined to be specific to immune hematopoietic cells. Interestingly, these 125 PID-associated genes were the most heavily concentrated in the monocyte, myeloid, B cell, T cell, and B and T cell categories. Protein-protein interaction network clustering produced 16 distinct clusters, with the largest and most highly interconnected clusters defined by immune cell surface, intracellular signaling, pattern recognition receptors, DNA repair, pro-proliferation, secreted immune, and extracellular matrix. When using categorical criteria for GSVA enrichment analysis, these 16 cluster signatures were able to sort a pool of 1,620 SLE patient whole blood transcriptomes into 12 subpopulations. These groupings may identify patients with different levels of immunologic activity or groups that may respond better to specific therapies. Cross-referencing 432 PID-associated genes across 14 SLE patient whole blood datasets revealed conserved enrichment for several functional gene categories, including IFN-stimulated genes, MHC-I, pattern recognition receptors, secreted and extracellular matrix, and secreted immune proteins. Together, these data analyses represent a large-scale, comprehensive bioinformatic review of the role of PID-associated genes in SLE, demonstrating that PID-defined genes are overexpressed in SLE patients and can be used to classify immunologic activity in lupus. These results provide a deeper understanding of the molecular basis of immune dysregulation in SLE. Further, these results may enable rapid identification and prioritization of potential drug targets that can be inhibited to suppress these dysregulated pathways (e.g., in SLE patients).

### Example 27: Analysis of Lupus Nephritis (LN) Gene Expression Reveals Dysregulation of Pathogenic Pathways Activated within Infiltrating Cells

Much is still to be learned about immune & inflammatory pathways in LN. A bioinformatic approach (LIMMA-DE & WGCNA) analyzed gene expression of LN biopsies microdissected for glomerulus and tubulointerstitum. Genes differing between LN & healthy individuals were interrogated for cell type specific gene signatures using GSVA validation of I- or T-Scope™ analysis of immune or nonimmune subsets. Podocytes are in WGCNA modules negatively correlated with WHO class. Genes were functionally characterized using BIG-C and pathways elucidated using IPA. LN has an immune cell signature in WGCNA modules positively correlated with WHO class (granulocytes, pDC, DC, myeloid cells, CD4, & CD8 Ts, Bs as well as pre- and post-switch PCs as indicated by IgM, IgD, and IgG1 HC genes). The presence of both Ig-κ & -λ, as well as VL genes suggests polyclonal activation. Chemokines that mediate lymphocyte organization and/or recruitment into lupus kidney are present. Cytokine (TNF, CD40L, IL1β, IL2, IL6, IL12, IL17, IL23, & IL27) & signaling (PI3K, NF-κB, NF-AT, and p70S6K) pathways as well as proliferation and HDAC activity are evident. IPA UPR analysis indicated ongoing signaling by cytokines such as TNF, IFNγ, IFNα, CD40L, IL1β, IL2, IL6, & IL17. Interestingly, connectivity analysis using LINCS/CLUE elucidated high priority drug targets such as IFNb (PF-06823859), IL12 (Ustekinumab) and S1PR (Fingolimod) that may prove to be good options for therapeutic intervention.

Lupus nephritis (LN) is a serious complication of SLE that affects about 20-40% of all lupus patients and leads to kidney damage, end-stage renal disease, and patient mortality. Despite advances in therapy, progression to end stage renal disease may not be affected. Therefore, it is important to re-consider the pathogenic mechanisms involved in LN as a basis for development of more effective therapies. A multi-pronged approach was performed to characterize LN via bioinformatic analysis of gene expression data obtained from kidney biopsies.

Genomic expression profiling data of LN patient biopsies, microdissected into glomerulus and tubulonterstitium (TI), was sourced from GSE32591 via the GEO database. Differentially expressed genes (DEGs) detected in LN-derived samples relative to samples from healthy individuals were interrogated for cell infiltrate composition using gene set variation analysis (GSVA) against a curated database of immune and non-immune cell type signatures (I-SCOPE, T-SCOPE). Weighted gene co-expression network analysis (WGCNA) was performed to generate gene modules correlated to clinical variables. DEGs were further functionally characterized using a curated immunity-specific gene functional category database (BIG-C) and IPA signaling pathway analysis software. Queries of the perturbation database (LINCS, Library of Integrated Network-Based Cellular Signatures) were used to identify possible upstream regulators of altered gene expression patterns in LN samples as well as to identify drugs that could reverse abnormal gene expression profiles.

WGCNA produced 6 gene modules (3 glomerulus, 3 TI) positively correlated with disease stage, as measured by WHO class. These modules were enriched in signatures for several immune cell types, including granulocytes, pDC, DC, myeloid cells, CD4+/CD8+ T cells, and B cells. Additionally, the presence of both IG-κ and -λ, as well as VL genes and detection of pre- and post-switch PCs as indicated by IgM, IgD, and IgG1 Ig Heavy Chain genes indicate polyclonal PC infiltration. Podocyte signatures were detected as enriched in WGCNA modules negatively correlated with WHO class. Chemokines and pathways that mediate lymphocyte proliferation, organization, and/or recruitment into lupus kidney tissue were detected as enriched via BIG-C and IPA analysis, including the cytokines TNF, IL1β, IL2, IL6, IL12, IL17, IL23, and IL27 and signaling pathways including CD40L, PI3K, NF-AT, and p70S6K. IPA upstream regulator analysis indicated ongoing signaling by cytokines such as TNF, IFNγ, IFNα, CD40L, IL1β, IL2, IL6, and IL17. Interestingly, connectivity analysis using LINCS elucidated high-priority drug targets such as IFNβ (PF-06823859), IL12 (Ustekinumab), and S1PR (Fingolimod) that may be suitable options for therapeutic intervention.

Bioinformatic analysis of LN gene expression highlighted several dysregulated signaling pathways that can form the targets of novel therapeutic strategies, and further elucidation of these signatures may enhance clinical surveillance and diagnosis of LN to improve patient outcomes.

SLE is a chronic and extremely polymorphic disease afflicting 1.5 million American patients, with more than 15,000 new cases each year. Lupus nephritis (LN) is a common serious complication of SLE, affecting 20-40% of all SLE patients and leading to severe kidney damage, end-stage renal disease, and patient mortality. LN is often initiated by immune complexes formed as a result of autoantibodies targeting self-antigens such as C1q and dsDNA. The deposition of these complexes throughout kidney glomeruli results in inflammation and infiltration of lymphocytes and phagocytic cells, leading to sustained tissue damage. Despite its prevalence, clinical outcomes for LN remain relatively poor while clinical surveillance and prediction of disease onset is difficult, highlighting the need to re-evaluate pathogenic mechanisms involved in LN as a basis for development of more effective therapies. Using systems and methods of the present disclosure, a multi-pronged approach was applied to characterize LN via bioinformatic analysis of gene expression data obtained from LN patient biopsies.

Gene expression data sourcing and processing were performed as follows. Publicly available DNA microarray data from microdissected kidney biopsies of 30 LN patients and 14 healthy controls were derived from NCBI Gene Expression Omnibus (GEO) under accession number GSE32591 (as described by Berthier et al., “Cross-Species Transcriptional Network Analysis Defines Shared Inflammatory Responses in Murine and Human Lupus Nephritis,” J. Immunol., 2012; which is incorporated herein by reference in its entirety). Raw data underwent background correction and GCRMA normalization resulting in log 2 intensity values compiled into expression set objects (e-sets).

Differential gene expression was performed as follows. Data from glomerular and tubulointerstitial samples were analyzed in two separate differential gene expression (DE) analyses. To maximize identification of DE genes, Affy chip definition files (CDFs) and BrainArray CDFs were used to create and annotate e-sets, analyzed separately, and merged. GCRMA normalized expression values were variance corrected using local empirical Bayesian shrinkage before calculation of DE values using the ebayes function in the BioConductor LIMMA package. Resulting p-values were adjusted for multiple hypothesis testing and filtered to retain DE probes with an FDR<0.2.

Weighted Gene Correlation Network Analysis (WGCNA) was performed as follows. Log 2 normalized microarray expression values, filtered for approximately the upper 50% of all probes, were used as input to WGCNA (V1.51). Resultant dendrograms of correlation networks were trimmed to isolate individual groups of probes using dynamic tree cutting and the deepSplit function, with the additional use of a partitioning around medoids function. Modules were given random color assignments and expression profiles summarized by a module eigengene (ME). ME values from each module were correlated to clinical metadata collected from GSE32591 by Pearson correlation.

Gene Set Variation Analysis (GSVA) was performed as follows. GSVA (V1.25.0) software package for R/Bioconductor was used as a non-parametric, unsupervised method for estimating the variation of pre-defined gene sets in patient and control samples of microarray expression data sets.

BIG-C® analysis was performed as follows. Biologically Informed Gene Clustering (BIG-C®) is a functional aggregating tool developed to understand the biological groupings of large lists of genes. Genes are sorted into 53 categories based on their most likely biological function and/or immune cellular localization based on information from multiple online tools and databases.

I-Scope™ and T-Scope™ analysis were performed as follows. I-Scope™ is a tool developed to identify hematopoietic cells through an iterative search of more than 17,000 genes identified in more than 50 microarray datasets. From this search, 926 genes met the criteria for restriction to hematopoietic lineages and were researched for immune cell specific expression in 27 hematopoietic sub-categories. T-Scope™ similarly identifies non-hematopoietic cells based on 10,000 tissue enriched genes and 8,000 cell line enriched genes from the human protein atlas, resulting in 42 tissue/cell specific categories. WGCNA module transcripts were entered into I-Scope™ and T-Scope™ to determine category overlap in each module. Odds ratios for category enrichment were calculated from number of transcripts in each category and overlap p values were determined using Fisher's exact test, and the negative log of the overlap p-value was plotted for all categories determined to be enriched (OR>1). Inclusion of the P-scope pathway category “Tissue Repair/Tissue Destruction” allows for prediction of the physiological effects of infiltrating populations identified via the other two tools, and therefore better characterization of each module's relationship to disease state.

IPA® upstream regulator analysis was performed as follows. IPA® Upstream Regulator (UR) analysis was utilized to identify possible upstream regulators of altered gene expression patterns in SLE samples.

Drug target prediction was performed as follows. Queries of the perturbation database from the Broad Institute (LINCS, Library of Integrated Network-Based Cellular Signatures) were utilized to identify possible upstream regulators of altered gene expression patterns in SLE samples as well as to identify compounds that induce gene expression profiles contrary to those patterns.

Module preservation was performed as follows. WGCNA results were compared to an independent SLE RNA-seq dataset using the module preservation function in the WGCNA software package (V1.51). A composite Zsummary statistic and an overlap p-value were calculated for each module. Modules with a Zsummary>2 were considered preserved. A module membership overlap table was generated and used to identify corresponding preserved modules between analyses.

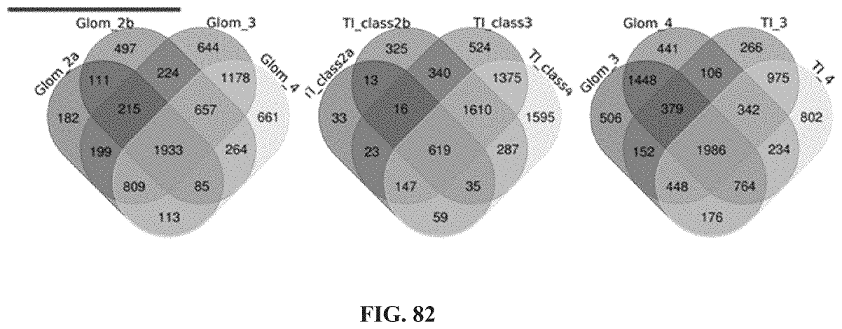

FIG. 82 shows results of LN differential gene expression. Microarray data from 30 LN patients and 14 healthy controls were processed by LIMMA to identify DE genes in microdissected glomeruli and TI from WHO classes 2a, 2b, 3, and 4. Each was analyzed independently in comparison to healthy controls. Substantial overlap was observed across WHO class within both glomerular and TI samples, as well as across glomerular and TI samples from proliferative-stage LN (classes 3 and 4).

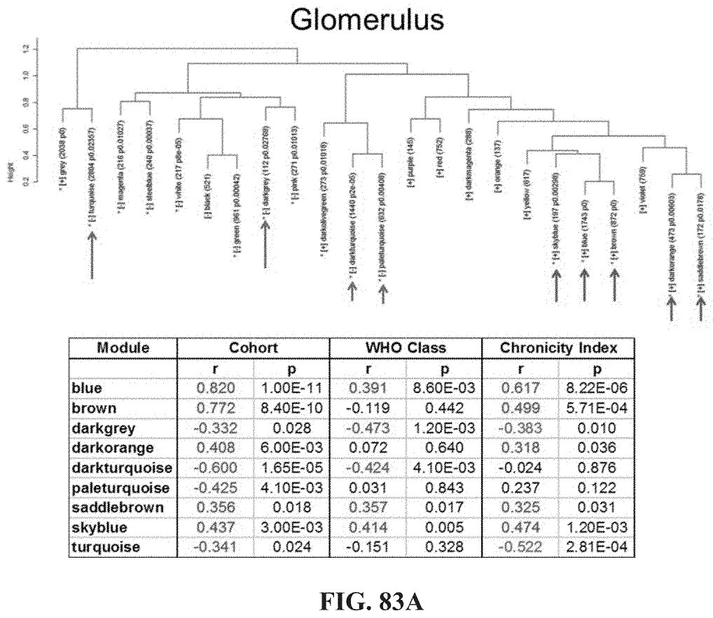

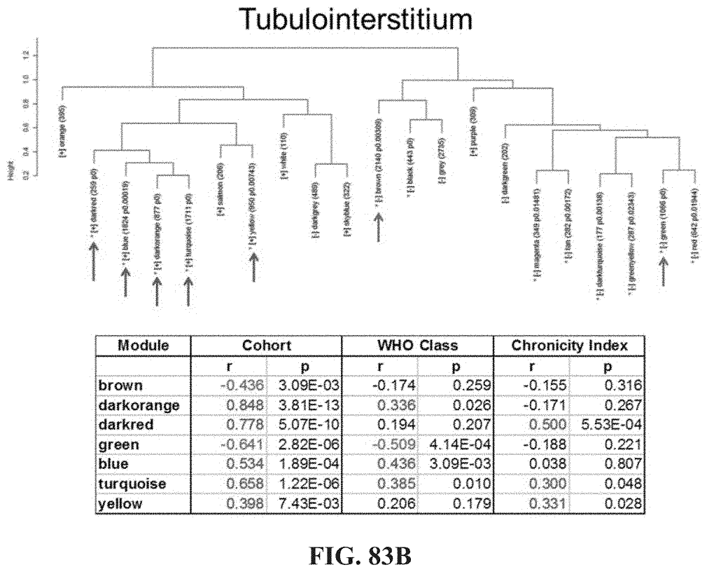

FIGS. 83A-83B show generation of WGCNA gene modules from LN glomerular and tubulointerstitium (TI) differential expression (DE) data and correlation to clinical covariates. Glomerular genes (FIG. 83A) and tubulointerstitium (TI) DE genes (FIG. 83B) were used as input to WGCNA. Resulting modules were chosen for further analysis based on significant correlation to clinical traits (cohort, WHO class, and chronicity index). Red arrows denote positively-correlated modules and blue arrows denote negatively-correlated modules within the module dendrogram. Modules are randomly assigned color name values by the WGCNA package and are independent from experiment to experiment.

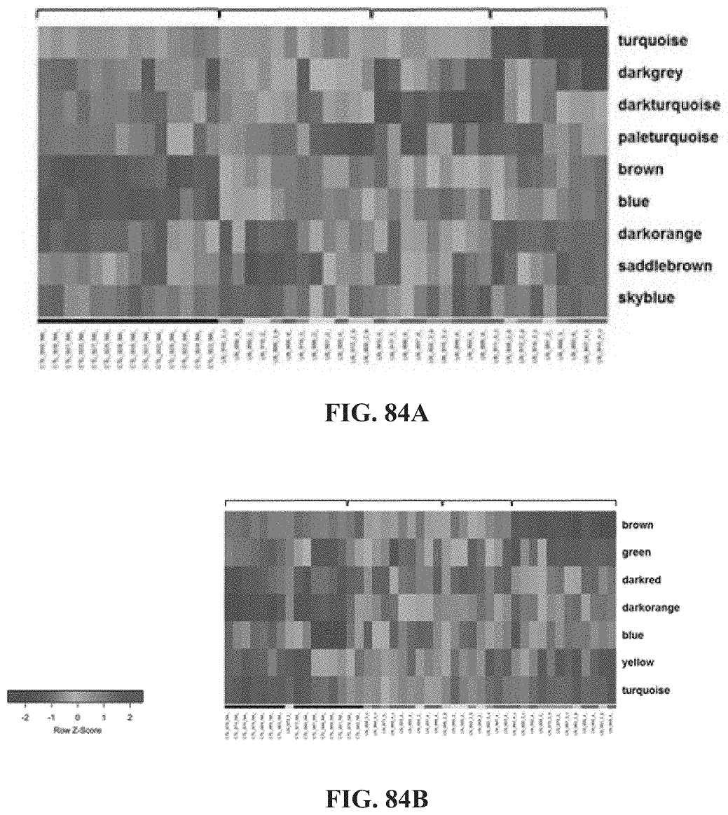

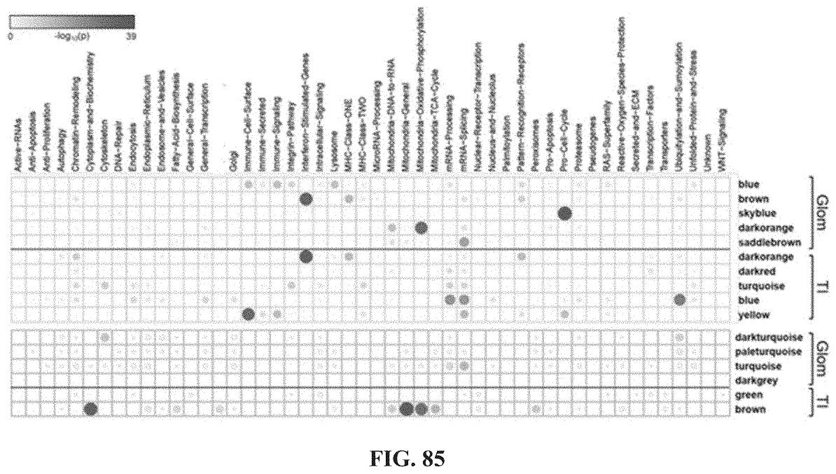

FIGS. 84A-84B show GSVA enrichment and sorting of LN patients against WGCNA module membership. Significantly correlated WGCNA modules were used as query gene lists for GSVA to evaluate efficacy in WHO class patient subtyping in glomerular (FIG. 84A) and TI (FIG. 84B) datasets. GSVA score heatmaps were arranged by row- and column-directed hierarchical clustering (black brackets above heatmaps represent algorithm-determined clusters). Strong correlations to cohort allowed confident sorting of healthy control samples from LN samples with varying success in identifying patients with specific levels of disease severity. Colored bar above sample names denotes WHO class (Light yellow, class 2a; dark yellow, class 2b; orange, class 3; red, class 4; black, healthy control).

FIG. 85 shows enrichment of functional categories in LN signatures via BIG-C®. Modules were characterized for patterns of member gene function via comparison to the BIG-C® database. Odds ratios for category enrichment were calculated for each module along with overlap p-value. Confidence of association as measured by the negative log of the overlap p-value was plotted for all categories determined to be enriched (odds ratio OR>1, magnitude represented by dot size). Positively-correlated modules are shown on the top 10 rows (5 rows Glom and 5 rows TI), and negatively-correlated modules are displayed on the bottom 6 rows (4 rows Glom and 2 rows TI).

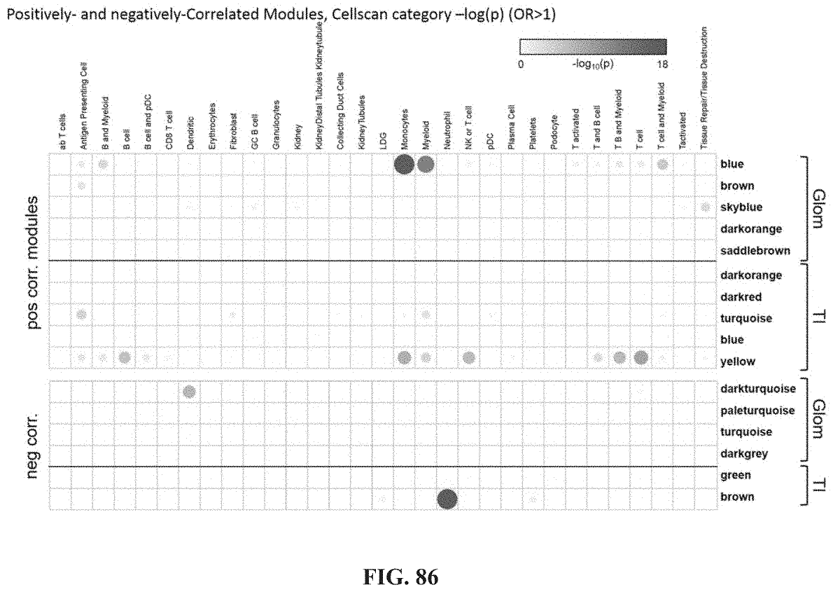

FIG. 86 shows enrichment of immune and tissue cell populations in LN signatures via I-Scope and T-Scope. Likely presence of immune and non-immune cell types was assessed via comparison to the T-Scope™ and I-Scope™ marker databases. Odds ratios and associated overlap p-values for category enrichment were calculated for each positively-correlated (top 10 rows (5 rows Glom and 5 rows TI)) and negatively-correlated (bottom 6 rows (4 rows Glom and 2 rows TI)) module, and the negative log of the overlap p-value was plotted for all categories determined to be enriched (OR>1).

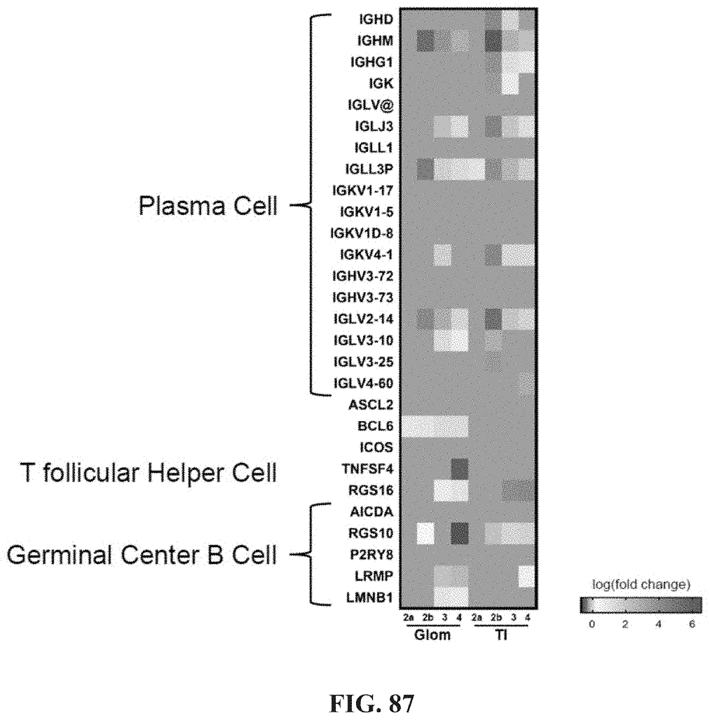

FIG. 87 shows expression of PC and GC indicator genes in LN. To more closely and specifically interrogate LN samples for the presence and role of PCs, DE genes from LN glomeruli and TI across WHO classes were filtered against signatures for core plasma cell function, T follicular helper cells, and germinal center B cells.

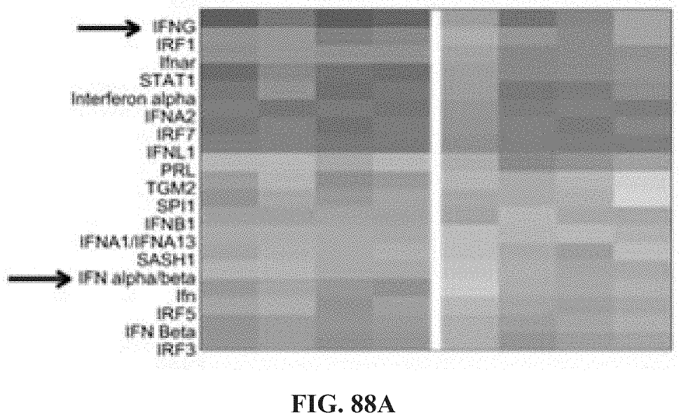

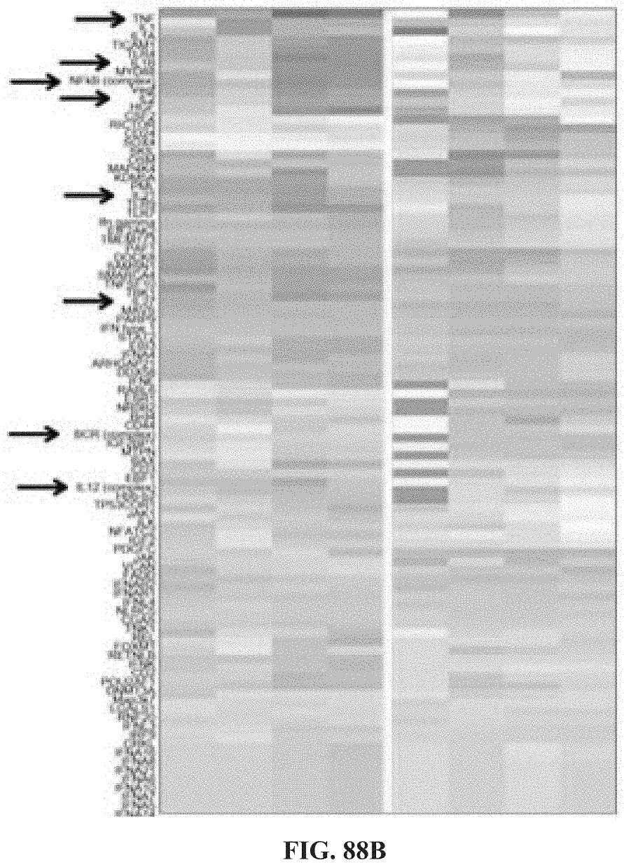

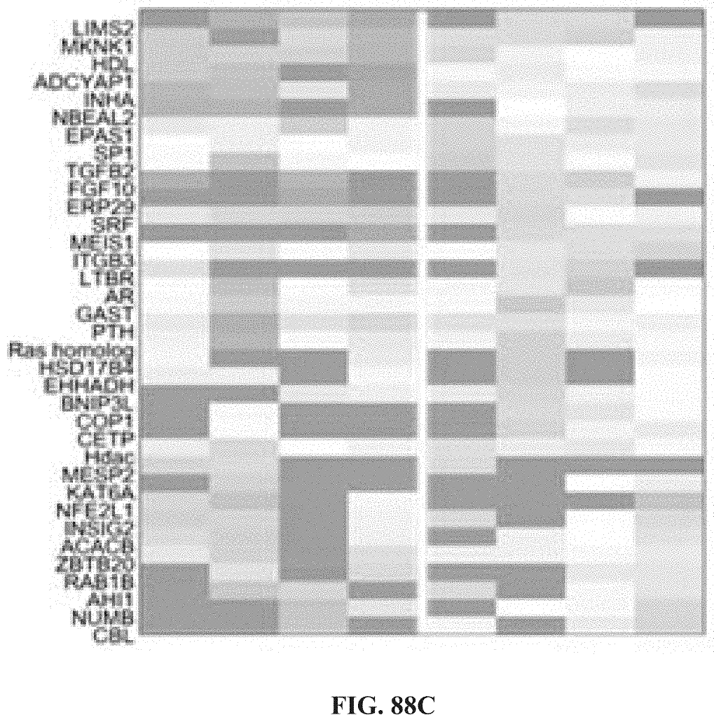

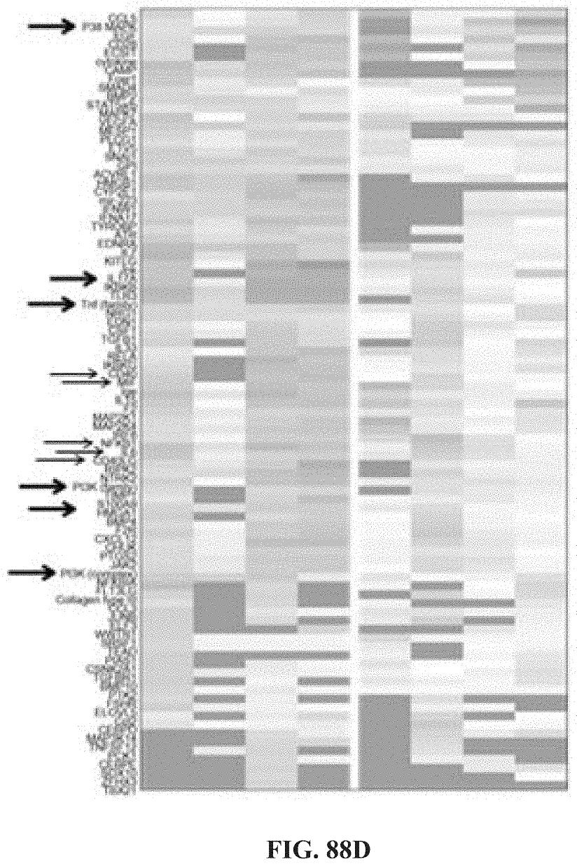

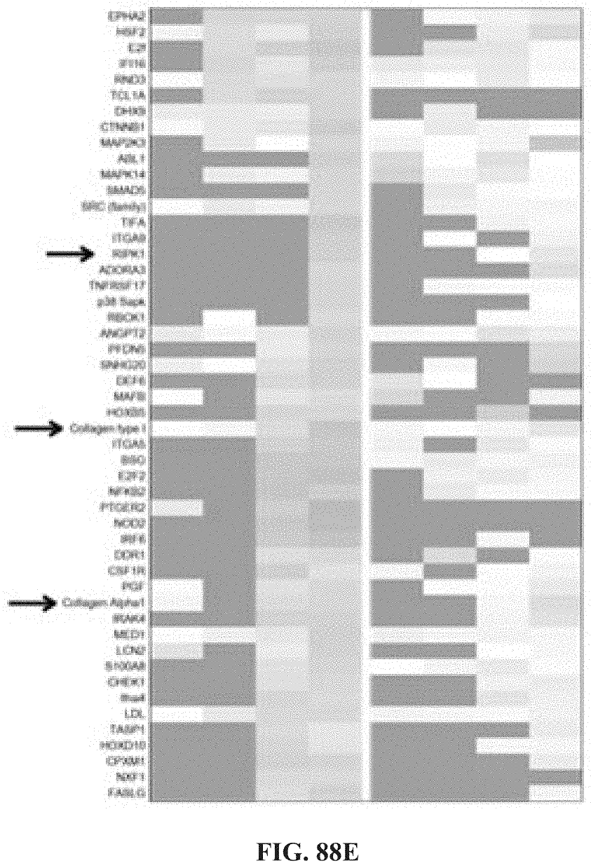

FIGS. 88A-88E show patterns of upstream regulator activation in LN. IPA® UR analysis of DE genes from glomerular and TI samples across WHO classes produces five blocks of interest (FIGS. 88A-88E, respectively) for identifying shared and unique immune, inflammatory, and cytokine/chemokine pathways between tissues and levels of LN severity (p<0.01).

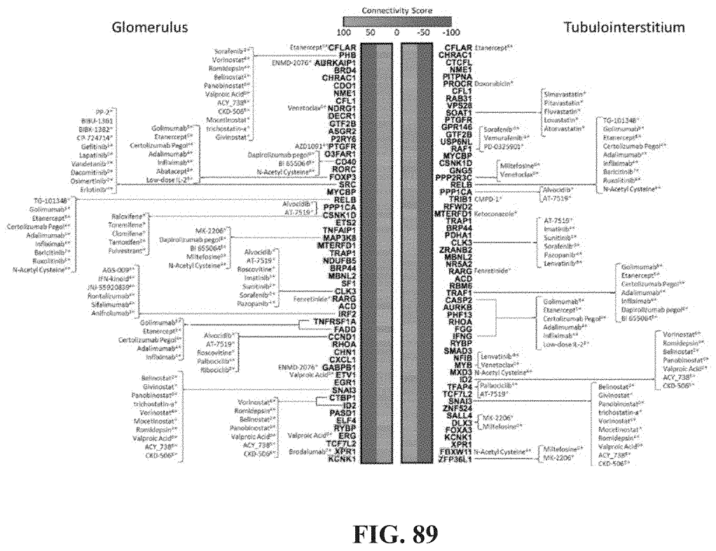

FIG. 89 shows LINCS analysis identifies priority targets and drugs in LN glomerular and TI via upstream regulators. DE genes were analyzed with the LINCS platform, which returns connectivity scores for genes and compounds based on similarity of input signatures to a database of experimental knockdown, overexpression, and drug treatment models. Genes with knockdown scores lower than −75 and overexpression scores over 50 were identified as upstream regulators and matched to direct- and indirect-acting drugs with scores lower than −75. Red asterisks denote FDA-approved drugs, blue asterisks denote drugs in development, superscript numbers denote CoLTs priority scores.

Table 49B shows that gene modules are preserved between human microarray and RNA-seq for LN. To verify biological significance and replicability of WGCNA modules, module preservation was performed against modules from an independent microdissected SLE kidney RNA-seq dataset. A module membership overlap table was generated separately for modules generated from glomerular tissue from each dataset and for modules generated from tubulointerstitial tissue from each dataset, and significantly preserved modules were identified by Fisher's exact test (p<0.05). Of the microarray modules that were significantly correlated to cohort and contained detectable functional enrichment signatures, two glomerulus modules (top of table) and four tubulointerstitium modules (bottom of table) showed significant overlap with one or more RNA-seq modules. Preserved modules were analyzed for enrichment of functional gene categories derived from the BIG-C database and ISCOPE/TSCOPE database and signatures that were shared between microarray and RNA-seq are shown in bold.

TABLE 49B
Glomerulus and tubulointerstitium module preservation between human microarray and RNA-seq for LN
Glomerulus Module Preservation

| | | | | | uA module | RNA-seq |
|---|---|---|---|---|---|---|
| Microarray | uA module | | | RNA-seq Module | ISCOPE/ | ISCOPE/ |
| module | correlations | RNA seq module(s) | uA module BIG-C | BIG-C | TSCOPE | TSCOPE |
| Blue | Pos corr cohort, WHO | Darkolivegreen | Immune cell surface, | Cytoskeleton, | Myeloid, | T, B, B- |
| | class, chronicity | (0.0191) | immune secreted, | immune cell | monocytes, | activated, |
| | | | immune signaling, | surface, immune | T, B | APCs |
| | | | integrin, IC | signaling, MHC-II | | |
| | | | signaling, lysosome, | | | |
| | | | MHC-I/II, mRNA | | | |
| | | | processing, PRR, | | | |
| | | | pro-apoptosis, | | | |
| | | | proteosome, RAS | | | |
| | | | superfamily and UPR | | | |
| Brown | Pos corr cohort, WHO | Brown | Mitochondrial DNA | Chromatin | APCs | pDC, |
| | class, chronicity | (0.000191) | to RNA, mRNA | remodeling, DNA | | platelets, |
| | | | splicing | repair, mRNA | | endothelia |
| | | | | splicing, pro cell | | |
| | | | | cycle, transcription | | |
| | | | | factors | | |
| | | Cyan | | mRNA processing, | | Neutrophils, |
| | | (0.0162) | | mRNA splicing, pro | | LDGs |
| | | | | cell cycle, | | |
| | | | | proteasome, UPR | | |

Tubulointerstitium Module Preservation

| | | | | | | RNA-seq |
|---|---|---|---|---|---|---|
| Microarray | RNA seq | uA module | | | uA module | ISCOPE/ |
| module | module(s) | correlations | uA module BIG-C | RNA-seq Module BIG-C | ISCOPE/TSCOPE | TSCOPE |
| Blue | Black | Pos corr | Chromatin remodeling, | Cytoskeleton, general | N/A | DCs, |
| | (0.048) | cohort, | endocytosis, general | cell surface, Golgi, | | monocytes/ |
| | | WHO class | transcription, Golgi, | integrin pathway, | | macrophages, |
| | | | mRNA processing, mRNA | secreted and ECM | | fibroblast, |
| | | | splicing, nucleus and | | | podocyte |
| | | | nucleolus, proteasome, | | | |
| | | | ubiquitylation and | | | |
| | | | sumoylation | | | |
| Brown | Blue | Neg corr | Cytoplasm and | Cytoplasm and | Kidney | Kidney |
| | (0.0413) | cohort | biochemistry, ER, | biochemistry, ER, | | |
| | | | fatty acid | fatty acid | | |
| | | | biosynthesis, | biosynthesis, | | |
| | | | glycolysis/ | glycolysis, lysosome, | | |
| | | | gluconeogenesis/ | mitochondria (general, | | |
| | | | pentose phosphate | DNA to RNA, ox phos, | | |
| | | | pathway, mitochondria | TCA), nucleus and | | |
| | | | DNA to RNA, | nucleolus, peroxisome, | | |
| | | | mitochondria general, | ROS, transporters, UPR | | |
| | Pink4 | | mitochondria ox phos, | Cytoskeleton, ER | | Lrythrocytes |
| | (0.016) | | mitochondria TCA | | | |
| | Sienna2 | | cycle, peroxisome | ER, PRRs | | Neutrophils |
| | (0.008) | | | | | |
| | Yellow2 | | | Mitochondria DNA to | | |
| | (0.0139) | | | RNA | | |
| Green | Blue3 | Neg corr | N/A | ER | N/A | |
| | (0.04) | cohort, | | | | |
| | Coral | WHO class | | Pro cell cycle, | | |
| | (0.0487) | | | ubiquitylation and | | |
| | | | | sumoylation | | |
| | Magenta3 | | | Melanosome | | |
| | (0.0445) | | | | | |
| | Salmon1 | | | Mitochondria general, | | Granulocytes |
| | (0.0161) | | | WNT signaling | | |
| Turquoise | Indianred3 | Pos corr cohort, | Chromatin remodeling, | Immune cell surface, | Antigen | T/B/Mono, |
| | (0.0363) | WHO class, | cytoskeleton, | immune signaling, | presenting cells, | Tregs, T |
| | | chronicity | endocytosis, integrin | integrin pathway, | myeloid cells | activated |
| | | | pathway, MHC-II, | PRRs, pro cell cycle | | |
| | Plum4 | | mRNA processing, | Immune secreted, pro | | CD8 T cells, |
| | (0.0496) | | mRNA splicing, UPR | apoptosis | | NKorT |
| | Sienna4 | | | Anti-apoptosis, | | |
| | (0.0307) | | | mitochondria DNA | | |
| | | | | to RNA, mitochondria | | |
| | | | | ox phos | | |
| | Turquoise | | | Chromatin remodeling, | | CD4 T cells |
| | (0.0068) | | | integrin pathway, | | |
| | | | | mRNA processing, | | |
| | | | | mRNA splicing | | |

WGCNA produced 6 gene modules (3 glomerulus, 3 TI) positively correlated with disease stage as measured by WHO class which contain enriched signatures for several immune cell populations and functional pathways.

Signs of tissue damage can be observed by signature enrichment analysis, as seen in enrichment of the tissue damage signature in a positively-correlated module and the podocyte signature in a negatively-correlated module.

Closer investigation of the PC signature found both IG-κ and -λ, as well as VL genes and detected pre- and post-switch PCs as indicated by IgM, IgD, and IgG1.

Chemokines and pathways that mediate lymphocyte proliferation, organization and/or recruitment into lupus kidney tissue were detected as enriched via BIG-C and IPA analysis, highlighting critical angles of therapeutic applications.

Connectivity analysis using LINCS elucidated high priority drug targets such as CFLAR, IFNg, CD40, RELB, SRC, TNFRSF1A, CCND1, and SNAI3 that may prove to be good options for therapeutic intervention.

Comparison of modules with an independent RNA-seq validation dataset revealed preservation of several modules from glomerulus and TI both by membership and functional enrichment.

### Example 28: GSVA-Based Data Analysis Tool (P-Scope)

A GSVA-based data analysis tool is developed for use in analyzing specific sets of gene pathways. The GSVA-based data analysis tool (e.g., P-Scope) may use a GSVA statistical test based tool using different sets of genes to analyze certain pathways. Such sets of genes may include, for example, human genes, mouse genes, or a combination thereof.

For example, an MS scoring test can be applied using an IL12-based set of genes (e.g., CCL5, CD40LG, CXCL10, CXCL12, CXCR3, GZMB, HAVCR2, HLX, IFNG, IL12A, IL12B, IL12RB1, IL12RB2, IL2, IL27, IRF4, MAPK14, PHF11, PRF1, STAT1, STAT4, STOM, TBX21, TYK2, IL2RA, MAP2K3, MAP2K6).

As another example, an MS scoring test can be applied using an IL23-based set of genes (e.g., ABCB1, BATF, CAMK4, CCL20, CCR6, CISH, CREM, CXCL1, IL12B, IL12RB1, IL17A, IL17F, IL21, IL22, IL23A, IL23R, IL26, IL6, IL6R, IKZF3, JAK2, KIT, KLRB1, MAF, PRKCA, PTPN13, RORA, RORC, STAT3, and TGFB1).

### Example 29: Analysis of Discoid Lupus Erythematosus (DLE) Gene Expression Reveals Dysregulation of Pathogenic Pathways Activated within Infiltrating Cells

Immune & inflammatory pathways in DLE skin are poorly understood. A bioinformatic approach (LIMMA-DE & WGCNA) was used to analyze skin biopsy gene expression to gain insight into precise pathogenic mechanisms involved. Genes differing between DLE & healthy individuals were interrogated for cell type specific gene signatures using GSVA validation of I-Scope or T-Scope® analysis of immune or non-immune subsets. Non-immune subsets (fibroblasts, keratinocytes, melanocytes and Langerhans cells) are in WGCNA modules negatively correlated with disease. Genes were functionally characterized using BIG-C® and pathways elucidated using IPA®.

DLE has an immune cell signature in WGCNA modules positively correlated with CLASI-A (DCs, myeloid cells, CD4+ & CD8+ T cells, γδ T cells, natural killer (NK) T cells, B cells, as well as pre-switch and post-switch PCs as indicated by IgM, IgD, and IgG1 HC genes). The presence of both Ig-κ & -λ as well as VL genes suggests polyclonal activation. Chemokines that mediate lymphocyte organization and/or recruitment into lupus skin are present. Cytokine (TNF, IFNγ, IFNα, CD40L, IL1β, IL2, IL6, IL12, IL17, IL23, and IL27) & signaling (PI3K, NF-κB, and mTOR) pathways as well as proliferation and HDAC activity are evident. IPA® UPR analysis indicated ongoing signaling by TNF, IFNγ, IFNα, CD40L, IL1β, IL2, IL6, IL12, IL17, IL23, and IL27.

Statistically significant WGCNA module preservation was observed between all three DLE datasets. Interestingly, connectivity analysis using LINCS/CLUE demonstrated high priority drug targets such as IKZF1/3 (lenlidomide) as well as CC-220, JAK1/2 (ruxolitinib) and HDAC6 (Ricolinostat) may prove to be good options for therapeutic intervention.

DLE is a chronic inflammatory autoimmune disease of the skin, characterized by scarring disk-shaped plaques, often on the face and neck. Most DLE patients only present cutaneous symptoms. However, DLE lesions can accompany other symptoms for approximately 20% of SLE patients. The precise molecular pathways underlying DLE pathogenesis have not been fully delineated. To obtain a more complete view of the pathologic processes involved in DLE, a comprehensive analysis of gene expression profiles was performed from DLE affected skin.

Gene expression data sourcing and processing were performed as follows. Publicly available microarray gene expression data was obtained from skin biopsy samples of three studies (GSE72535, GSE81071, & GSE52471). The studies included 9 DLE and 8 Control (GSE72535), 26 DLE and 7 Control (GSE81071), and 7 DLE and 10 Control (GSE52471). Raw data underwent background correction and GCRMA normalization resulting in log 2 intensity values compiled into expression set objects (e-sets).

Differential gene expression analysis was performed as follows. For each dataset, the skin biopsy data was analyzed as a separate differential gene expression (DE) analysis. To maximize identification of DE genes, Affy chip definition files (CDFs) and BrainArray CDFs were used to create and annotate e-sets, analyzed separately, and merged. For GSE72535, the Illumina CDF was used in one DE analysis. GCRMA normalized expression values were variance corrected using local empirical Bayesian shrinkage before calculation of DE values using the ebayes function in the BioConductor LIMMA package. Resulting p-values were adjusted for multiple hypothesis testing and filtered to retain DE probes with an FDR<0.2.

Weighted Gene Correlation Network Analysis (WGCNA) was performed as follows. Log 2 normalized microarray expression values, filtered for approximately the upper 50% of all probes, were used as input to WGCNA (V1.51). Resultant dendrograms of correlation networks were trimmed to isolate individual groups of probes using dynamic tree cutting and the deepSplit function, with the additional use of a partitioning around medoids function. Modules were given random color assignments and expression profiles summarized by a module eigengene (ME). ME values from each module were correlated to clinical metadata collected from each dataset by Pearson correlation.

Gene Set Variation Analysis (GSVA) was performed as follows. GSVA (V1.25.0) software package for R/Bioconductor was used as a non-parametric, unsupervised method for estimating the variation of pre-defined gene sets in patient and control samples of microarray expression datasets.

BIG-C analysis was performed as follows. Genes were sorted into 53 categories based on their most likely biological function and/or immune cellular localization based on information from multiple online tools and databases.

I-Scope™ and T-Scope™ analyses were performed as follows. The I-Scope™ tool was used to identify hematopoietic cells through an iterative search of more than 17,000 genes identified in more than 50 microarray datasets. From this search, 926 genes met the criteria for restriction to hematopoietic lineages and were researched for immune cell specific expression in 27 hematopoietic sub-categories. The T-Scope™ tool similarly was used to identify non-hematopoietic cells based on 10,000 tissue enriched genes and 8,000 cell line enriched genes from the human protein atlas, resulting in 42 tissue/cell specific categories. WGCNA module transcripts were entered into I-Scope™ and T-Scope™ to determine category overlap in each module. Odds ratios for category enrichment were calculated from number of transcripts in each category and overlap p values were determined using Fisher's exact test.

IPA Canonical Pathway and Upstream Regulator Analysis was performed as follows. IPA® Canonical Pathway analysis was utilized to identify possible pathways underlying the altered gene expression patterns in DLE samples. Additionally IPA® Upstream Regulator (UR) analysis was utilized to identify possible upstream regulators of altered gene expression patterns in DLE samples.

Module Preservation analysis was performed as follows. Consistency across the three WGCNA results was measured using the module preservation function in the WGCNA software package (V1.51). The three datasets were compared pairwise. In each comparison, a composite statistic, Z_{summary}, was calculated for each module, based on several measures of network similarity. Modules with a Z_{summary}>2 were considered preserved. A module membership overlap table was used to identify corresponding preserved modules between analyses, with a Fisher's exact test determining significant overlaps of modules between two given analyses.

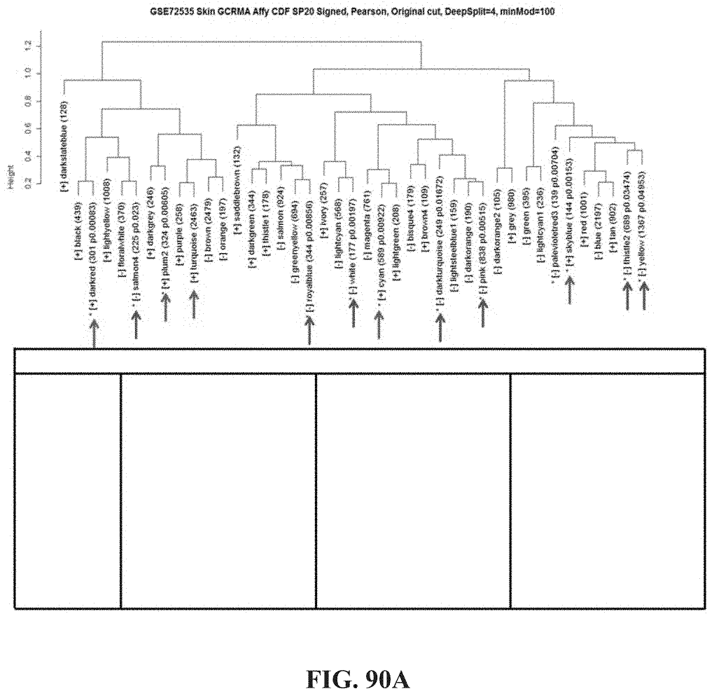

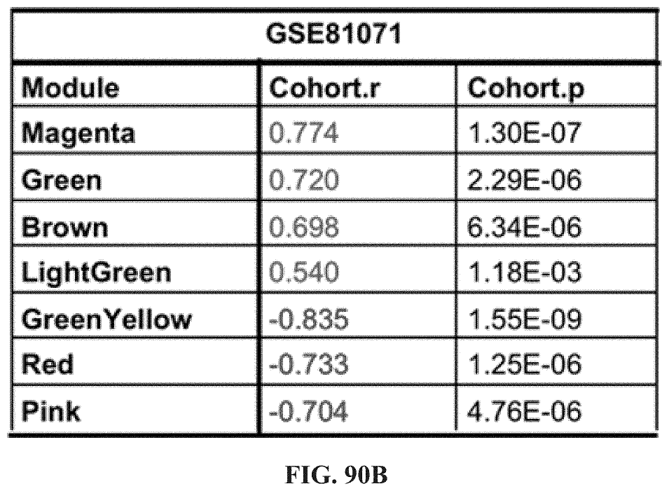

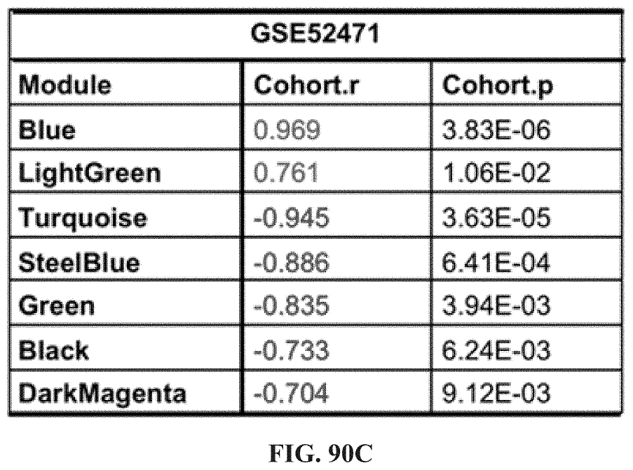

FIGS. 90A-90C show an example of performing WGCNA to identify modules with significant correlations to clinical variables. Performing WGCNA identified 41 modules for GSE72535, 23 modules for GSE81071, and 30 modules for GSE52471. In GSE72535, 12 modules were significantly correlated to CLASI.A or cohort (5 positively and 7 negatively). In GSE81071 and GSE52471, 7 modules were significantly correlated to cohort (GSE81071: 4 positively and 3 negatively; GSE52471: 2 positively and 5 negatively).

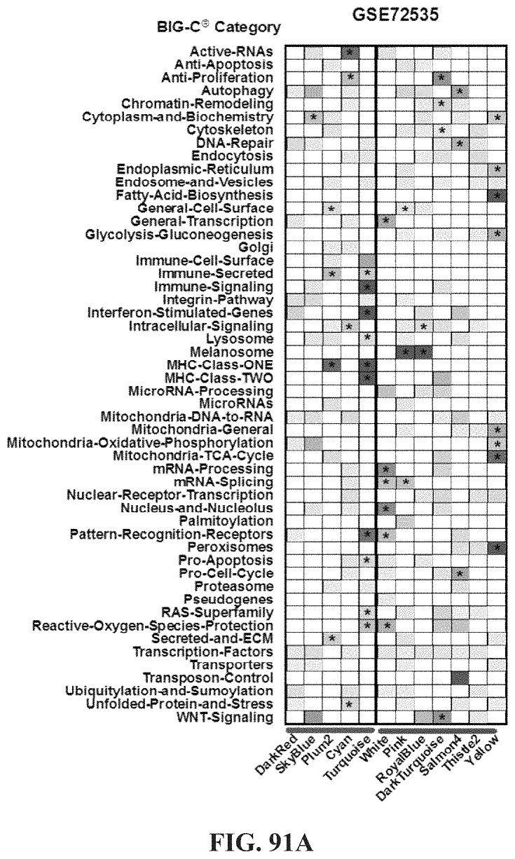

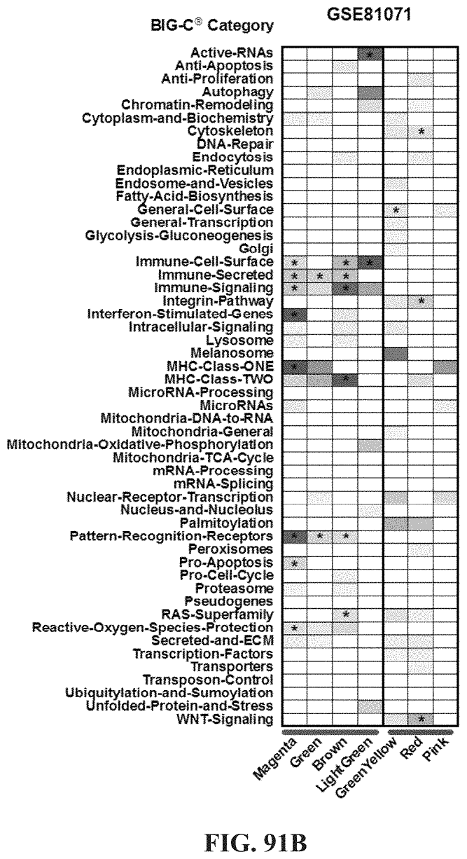

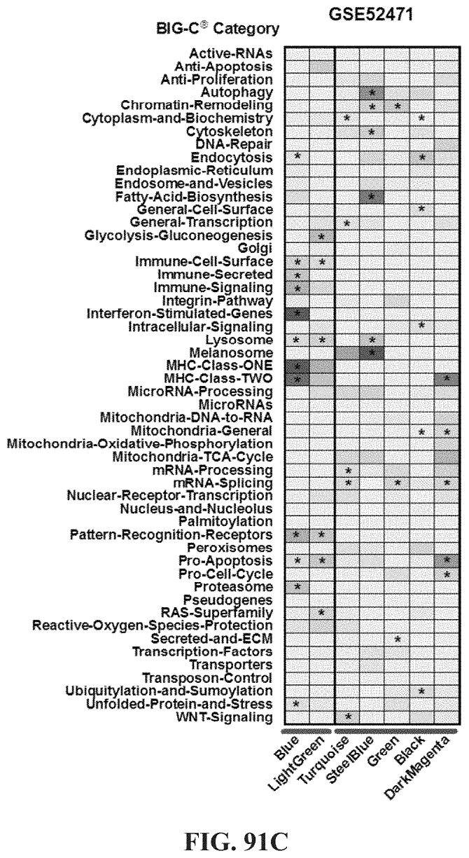

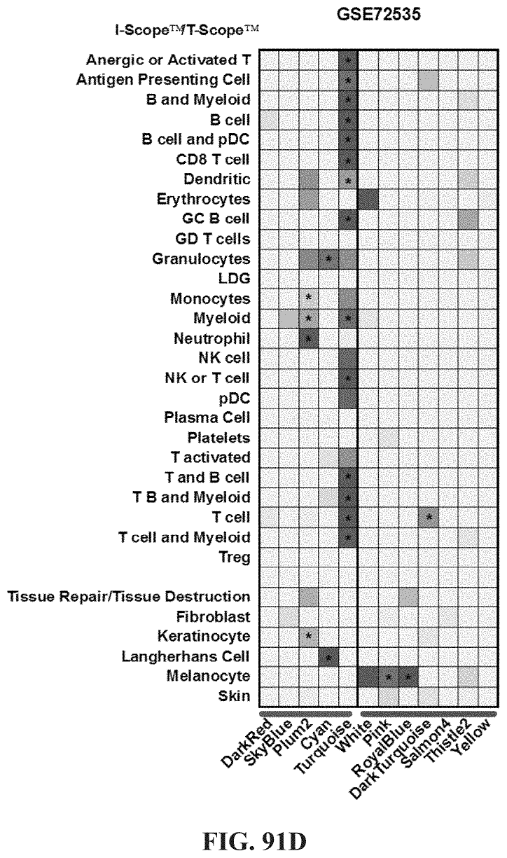

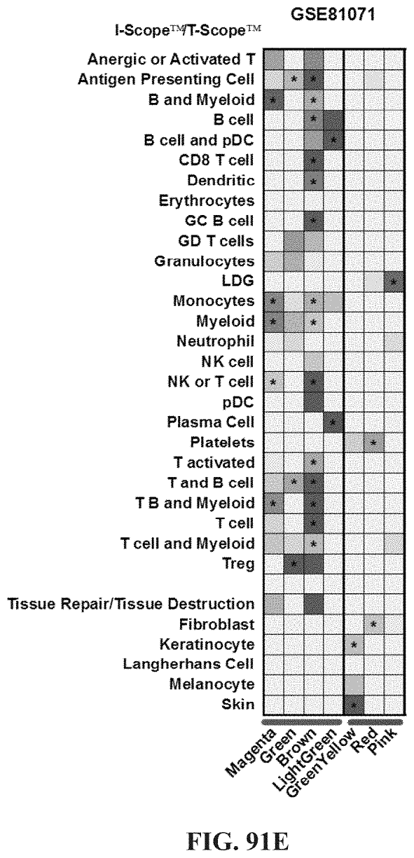

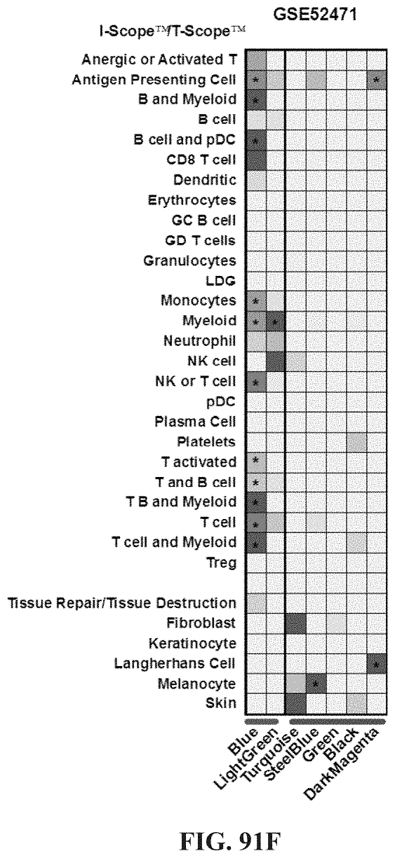

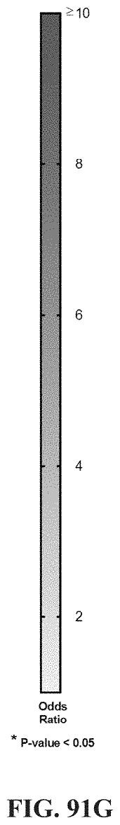

FIGS. 91A-91G show an example of WGCNA modules interrogated using BIG-C® functional characterizations as well as I-Scope™ and T-Scope™ for specific cellular subsets. DLE-associated modules identified in WGCNA are characterized by BIG-C® (FIGS. 91A-91C) and I-Scope™ and T-Scope™ (FIGS. 91D-91F). Odds ratios above 1 are shown, and Fisher's exact tests with p-values below 0.05 are indicated with an asterisk (FIG. 91G). Consistent enrichment of several categories, including immune signaling, pattern recognition receptors, and pro-apoptosis, was seen across all three analyses. Additionally, a clear immune signature, including antigen presenting cells, T cells, and myeloid cells, was observed in positively correlated modules.

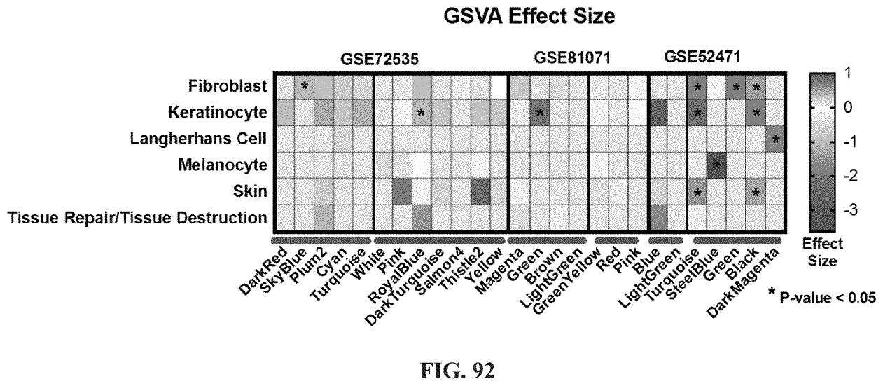

FIG. 92 shows an example of expression of tissue-specific signatures in WGCNA modules interrogated by GSVA. Gene Set Variation Analysis (GSVA) was performed to find enrichment of tissue specific gene signatures in each module. An enrichment score for a given signature in a module was calculated for each subject. To compare the scores of the controls and patients, a Cohen's d effect size was calculated, and significant enrichment was determined by a student's t-test.

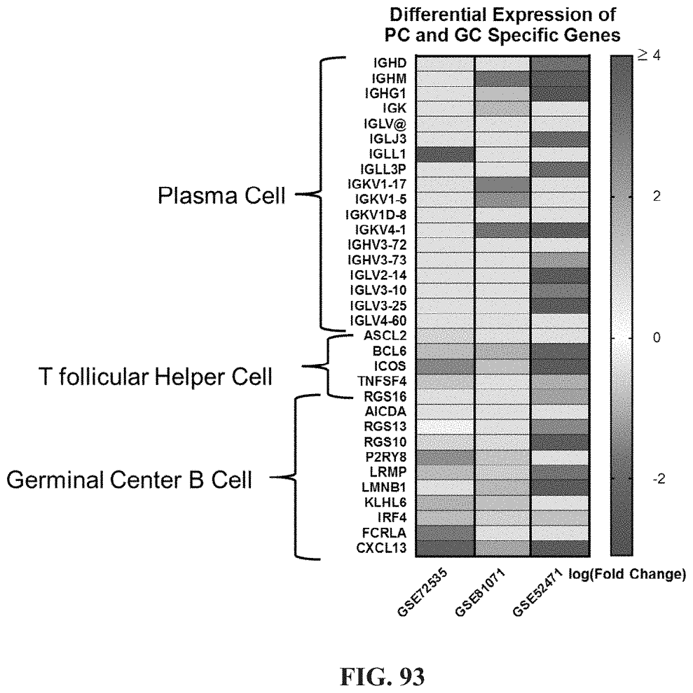

FIG. 93 shows an example of expression of PC and GC indicator genes in DLE. To more closely and specifically interrogate DLE samples for the presence and role of PCs, DE genes from each dataset were filtered against signatures for core plasma cell function, T follicular helper cells, and germinal center B cells.

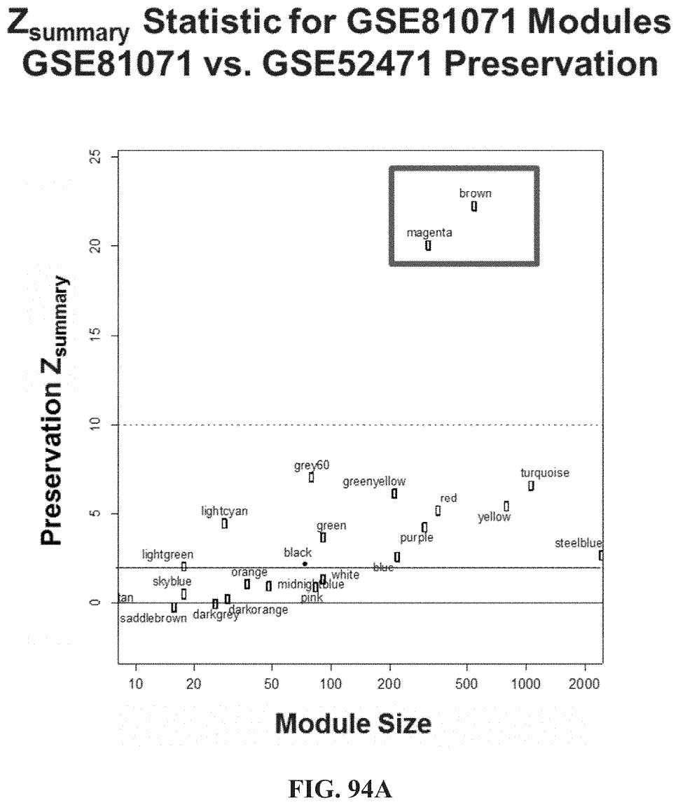

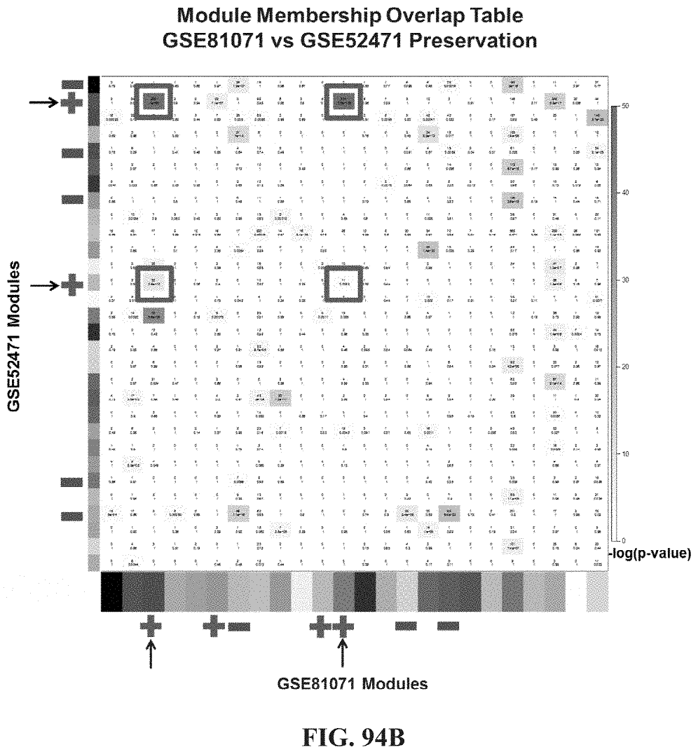

FIGS. 94A-94B show an example of WGCNA modules statistically preserved between three analyses. Module preservation was performed for each pairwise combination of datasets. The preservation Zsummary statistic was used to determine significant preservation. As shown in FIG. 94A, a representative example of the WGCNA modules from GSE81071 in the preservation analysis between GSE81071 and GSE52471. As shown in FIG. 94B, the overlap p-value (Fisher's exact test) was used to determine specific module associations between datasets. Interestingly, the analyses consistently showed the preservation of the two positively correlated modules in each dataset (Turquoise and Plum2 in GSE72535, Brown and Magenta in GSE81071, and Blue and LightGreen in GSE52471).

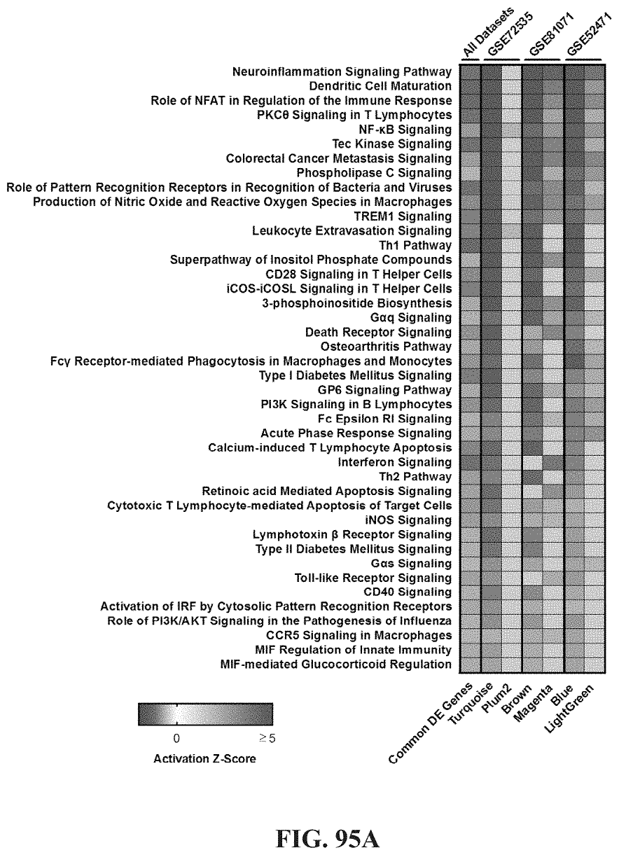

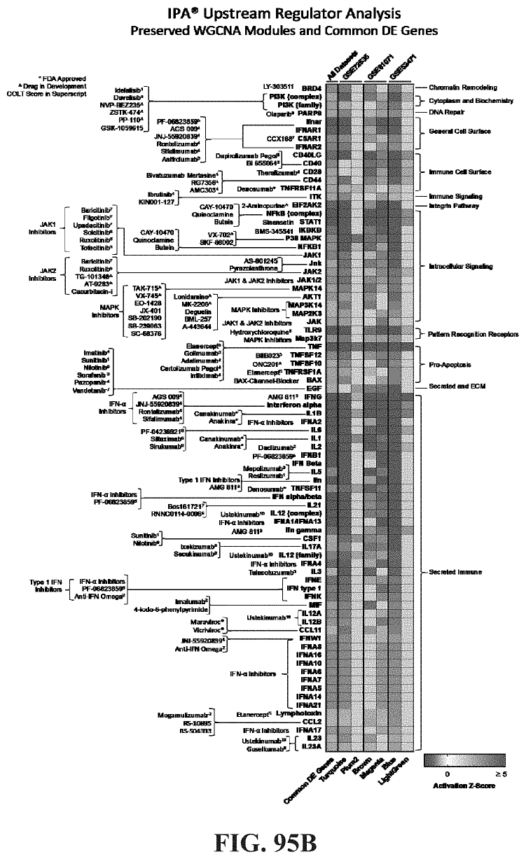

FIGS. 95A-95B show an example of IPA canonical pathway and upstream regulator (UR) analysis. IPA® canonical pathway and upstream regulator analysis was performed. The analysis compared DE genes common to all three datasets and the 6 preserved DLE-associated WGCNA modules. As shown in FIG. 95A, canonical pathways predicted to be significantly activated or inhibited in both DE transcripts and at least one module from each dataset were shown. As shown in FIG. 95B, a total of 224 URs were significantly activated or inhibited in both the DE transcripts and at least one module from each dataset. The 84 URs targeted by existing drugs are shown and organized by BIG-C™ category. Canonical pathways and upstream regulators were considered significant if |Activation Z-Score|≥2.

WGCNA identified several modules in each dataset that significantly correlated to disease. Notably, two positively correlated modules in each dataset were significantly preserved across all three analyses.

Signs of tissue damage can be observed by signature enrichment analysis, as seen in enrichment of skin specific cellular signatures in negatively correlated modules.

Closer investigation of the PC signature found both IG-κ and -λ, as well as VL genes and detected pre- and post-switch PCs as indicated by IgM, IgD, and IgG1.

Chemokines and pathways that mediate lymphocyte proliferation, organization and/or recruitment into DLE cutaneous tissue were detected as enriched via IPA® analysis, highlighting critical angles of therapeutic attack.

Specifically, several IPA® URs were also high priority drug targets such as IFNγ, CD40, IL12, TNFRSF1A, IFNα, and JAK/STAT pathways that may prove to be good options for therapeutic intervention.

### Example 30: E-Genes Identified Via Transancestral SNP Mapping and Gene Expression Analysis Reveal Novel Targeted Therapies for African-American and European-American SLE Patients

Systemic lupus erythematosus (SLE) in African-Americans (AA) is more prevalent, more severe and associated with an increased burden of co-morbidities compared to European-American (EA) populations. Genome-wide association studies (GWAS) have linked many single nucleotide polymorphisms (SNPs) to SLE. For example, large-scale transancestral association studies of SLE may be performed to identify ancestry-dependent and independent contributions to SLE risk. Such findings may be extended to include a transancestral analysis linking SLE-associated SNPs to candidate-causal E-Genes specific to AA and EA populations and differential gene expression in these populations with the goal of matching genetic and genomic disease characteristics with available treatments unique to each ancestral group.

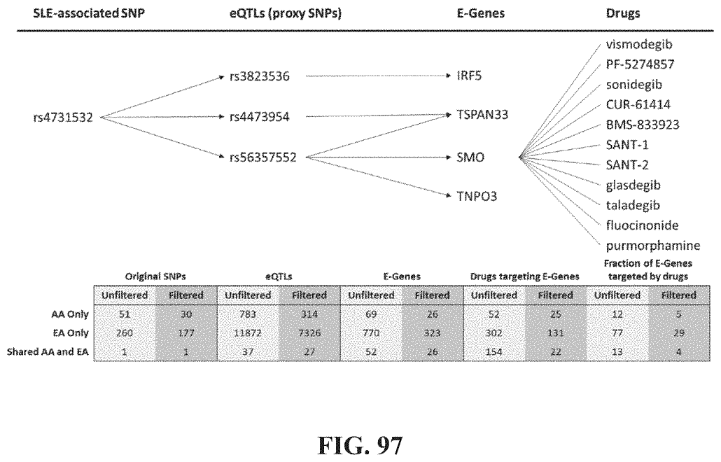

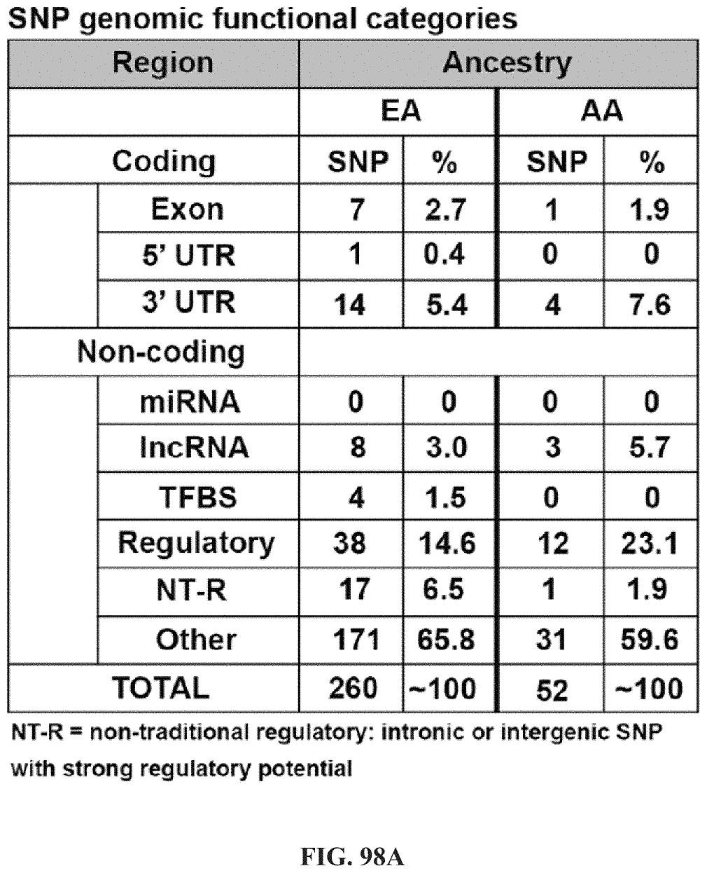

SNP proxies in linkage disequilibrium with SLE-associated SNPs were compared with known expression quantitative trait loci (eQTLs) contained in the GTEx (version 6) database. E-QTLs and their associated E-Genes were divided by ancestry and compared to differentially expressed (DE) genes from multiple SLE gene expression datasets. For both ancestral groups, E-Gene lists were examined for the significant enrichment of BIG-C categories and IPA (Qiagen) Canonical Pathways to predict novel upstream regulators (UPRs). For visualization and clustering analysis, STRING-generated networks of DE E-Genes were imported into Cytoscape (version 3.6.1) and partitioned with the community clustering (GLay) algorithm via the cluster-Maker2 (version 1.2.1) plugin. Finally, drug candidates targeting E-Genes, DE genes, and UPRs were identified using CLUE, REST, API, IPA, and STITCH (version 5.0; stitch.embl.de). The process of unpacking an SLE-associated SNP is shown in FIG. 97.

E-QTL and DE gene queries of GTEx were combined and newly predicted E-Genes were pooled by ancestry. Here, we identify 52 SNPs with eQTLs unique to AA ancestry, 260 SNPs unique to EA ancestry, and 1 SNP shared between AA and EA ancestries. Together, these SNPs identified a total of 891 distinct E-Genes associated with both ancestral groups. In studies comparing E-Genes to SLE DE data sets, 516 EA E-Genes were differentially expressed compared to 48 AA E-Genes. Comparison with various drug candidate databases resulted in the identification of 12 drugs targeting genes specific for AA, 77 drugs specific for EA genes, and 13 shared between EA and AA genes. Predicted EA-specific drugs include hydroxychloroquine and drugs-in-development targeting CD40LG, CXCR1 and CXCR2; whereas AA-specific drugs include HDAC inhibitors, retinoids, and drugs targeting IRAK4 and CTLA4. Drugs targeting E-Genes and/or pathways shared by EA and AA include ibrutinib, ruxolitinib, and ustekinumab.

The ancestral SNP-associated E-Genes and gene expression profiles outlined here illustrate fundamental differences in lupus molecular pathways between AA and EA. These results indicate that unique sets of drugs may be particularly effective at treating lupus within each ancestral group.

### Example 31: E-Genes Identified Via Transancestral SNP Mapping and Gene Expression Analysis Reveal Novel Targeted Therapies for African-American and European-American SLE Patients

Systemic lupus erythematosus (SLE) is a heterogeneous autoimmune disease that disproportionately affects subjects (e.g., women) of African-Ancestry (AA) compared to their European-Ancestral (EA) counterparts. This disparity may be further complicated by the fact that FDA-approved treatments for SLE, such as belimumab, may not provide a significant therapeutic benefit in SLE-affected AA subjects (e.g., women). Therefore, the genetic components unique to each ancestry were determined, and then these genetic targets were matched with novel drug candidates to help establish ancestry-specific therapies. To accomplish this, genetic variations or “polymorphisms” unique to each ancestral population were identified and then mapped to specific genes. Genes and their associated pathways may then be applied to multiple drug screening databases. This analysis resulted in the identification of drugs targeting genes specific for AA, EA, and genes common to both AA and EA ancestries. Together, these studies help provide a precision-medicine foundation for the establishment of patient-specific therapies and interventions for SLE.

Systemic lupus erythematosus (SLE) in African-Ancestry (AA) populations is more prevalent, more severe, and associated with an increased burden of co-morbidities compared to European-Ancestry (EA) populations. SLE is strongly influenced by genetic factors, and recent candidate gene and genome-wide association studies (GWAS) have linked many single nucleotide polymorphisms (SNPs) to SLE. Understanding the functional mechanisms of causal genetic variants underlying SLE may provide a key to identifying ancestry-specific molecular pathways and therapeutic targets relevant to disease mechanisms. Although GWAS have achieved great success in mapping disease loci, in polygenic autoimmune diseases, many GWAS findings have failed to impact clinical practice. Large-scale transancestral association studies of SLE may be performed to identify ancestry-dependent and independent contributions to SLE risk. Here, we link SLE-associated variants from diverse ancestral populations to biologically relevant genes (E-Genes) via the GTEx database. This analysis has led to the identification of 69 and 770 E-Genes specific for AA and EA respectively, with 52 E-Genes shared between AA and EA ancestries. We then applied a comprehensive systems biology approach using available bioinformatics and pathway analysis tools (e.g. IPA, STRING) to identify the genetic drivers of gene expression networks and key genes within SLE-associated biological pathways, including upstream and downstream regulators. Newly predicted E-Genes and their regulators were then coupled to SLE differential expression (DE) datasets to map candidate molecular pathways and available treatments unique to each ancestral group. Together, these genetic and gene expression analyses clarify the fundamental differences in lupus molecular pathways between ancestral populations and help identify novel drug candidates that may uniquely impact SLE in EA and AA populations.

Identification of SLE-associated SNPs, eQTLs, and E-Genes was performed as follows. A set of single nucleotide polymorphisms significantly associated with SLE in AA (2,970 cases; 2,452 controls) and EA (6,748 cases; 11,516 controls) cohorts was obtained (as described by, for example, Langefeld et al., “Transancestral mapping and genetic load in systemic lupus erythematosus,” Nature Communications, 8:16021, Jul. 17, 2017, DOI: 10.1038/ncomms16021; which is incorporated herein by reference in its entirety). SNP proxies (raggr.usc.edu) in linkage disequilibrium (r2>0.5) with these SLE-associated SNPs were then determined, using the European (CEU) population as background for EA SNPs and the African (YRI) population for AA SNPs. Expression quantitative trait loci (eQTLs) were then identified using GTEx (version 6). These eQTLs and their associated eQTL expression genes (E-Genes) were divided into an AA group and an EA group, dependent on the ancestry of the original SLE-associated SNP from which the eQTL was obtained.

SNP genomic functional categories were obtained as follows. The Variant Effect Predictor tool available on the Ensembl genome browser 93 (www.ensembl.org) was used for SNP annotation information. SNPs within 5 kilobases (kb) upstream of transcription start sites (TSS) were considered upstream regions, and SNPs within 5 kb downstream of transcription termination sites (TTS) were considered downstream regions. The online resource tools RegulomeDB (regulomedb.org) and HaploReg (version 4.1; pubs.broadinstitute.org/mammals/haploreg/haploreg.php) were also used to identify DNA features and regulatory elements, and to assess regulatory potential.

E-Gene functional gene set analyses were performed as follows. For both ancestral groups, E-Gene lists were examined and classified using a variety of techniques, including PANTHER GO slim (Protein ANalysis THrough Evolutionary Relationships, part of the Gene Ontology (GO) reference genome project; pantherdb.org v. 13.1) and statistical enrichment of BIG-C™ (Biologically Informed Gene Clustering, v. 4.3) categories. STRING (string-db.org, v. 10.5) and CytoScape (v. 3.6.1) aided genetic pathway identification and visualization, respectively. E-Genes were also compared with differential expression data gathered from SLE gene expression studies, including E-GEOD-24706, EMTAB2713, FDABMC3, GSE4588, GSE10325, GSE22098, GSE29536, GSE32591, GSE36700, GSE38351, GSE39088, GSE45291, GSE49454, GSE50772, GSE52471, GSE61635, GSE72535, GSE81071, GSE81622, GSE88884, and GSE100093. Differential expression log fold changes were determined for probes with false discovery rate (FDR)<0.2. This differential expression data was also used in conjunction with IPA® (Qiagen) to predict upstream regulators (URs) of E-Genes.

Drug candidate identification and CoLT scoring were performed as follows. Drug candidates were identified using CLUE (clue.io/repurposing), IPA, and STITCH (Search Tool for Interacting CHemicals; stitch.embl.de). Where information was available, drugs were assessed by CoLTS (Combined Lupus Treatment Scoring) (as described by, for example, Grammer et al., “Drug repositioning in SLE: crowd-sourcing, literature-mining and Big Data analysis,” Lupus, 2016 Sep., 25(10):1150-70, DOI: 10.1177/0961203316657437; which is incorporated herein by reference in its entirety) to rank potential drug candidates for repositioning in SLE. Each of these tools includes either a programmatic method of matching existing therapeutics to their targets or a list of drugs and targets for achieving the same end.

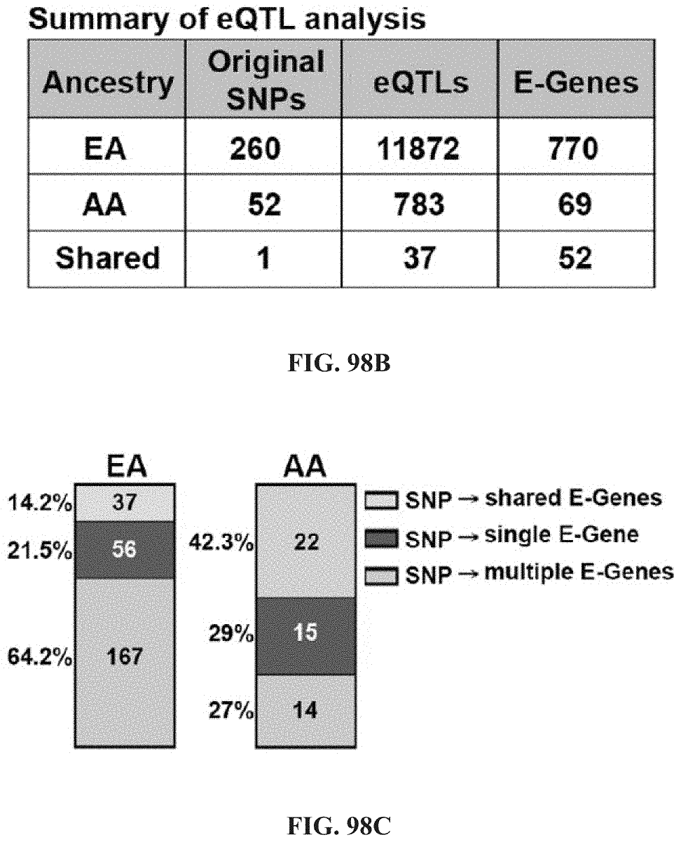

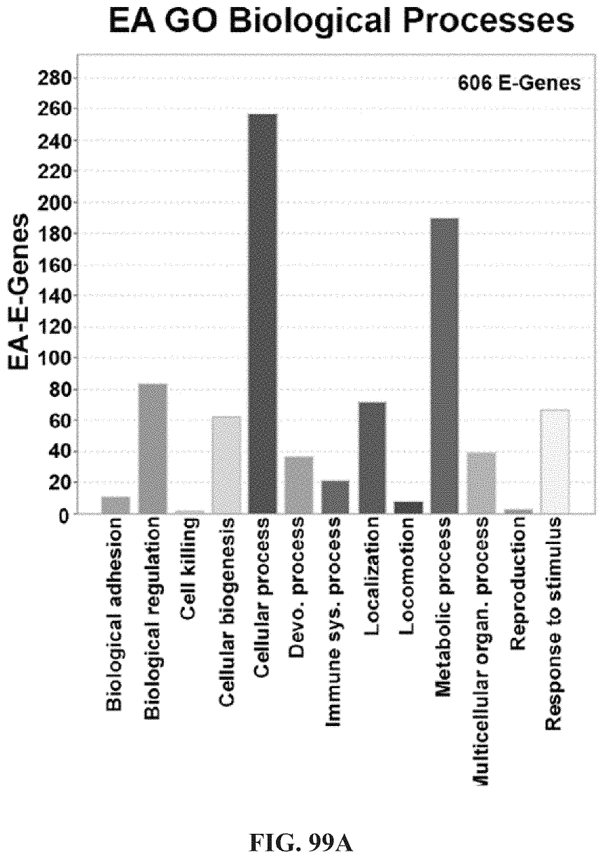

FIGS. 98A-98C show an example of mapping SNP associations to eQTLs and E-Genes, in accordance with disclosed embodiments. FIG. 98A shows a distribution of genomic functional categories for EA and AA SNP sets. “NT-R” is defined as Non-Traditional Regulatory: intronic or intergenic SNPs exhibiting strong regulatory potential, indicated by DNAse hypersensitivity, location within protein binding sites, and evidence of epigenetic modification. “Other” non-coding regions include introns, intergenic regions, within 5 kb upstream of transcription start sites, and within 5 kb downstream of transcription termination sites. FIG. 98B shows a summary of eQTL analysis. SLE-associated SNPs identify multiple eQTLs linked to E-Genes in the GTEx database. eQTLs and their associated E-Genes were divided into European ancestry (EA) and African ancestry (AA) groups, depending on the ancestral origin of the original SLE-associated SNP. Shared E-Genes are derived from SNPs common to both EA and AA ancestries. FIG. 98C shows the number of EA and AA SNPs mapping to single E-Genes, multiple E-Genes, or shared E-Genes.

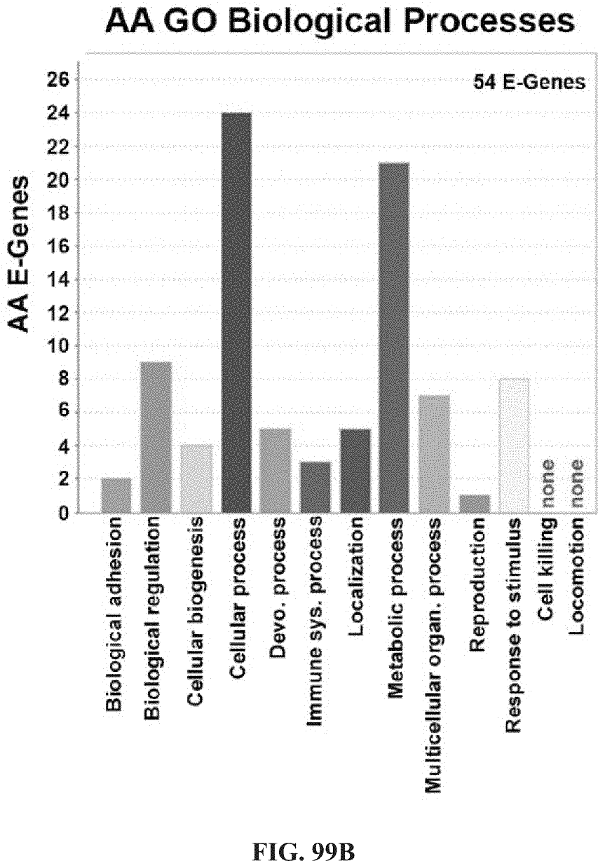

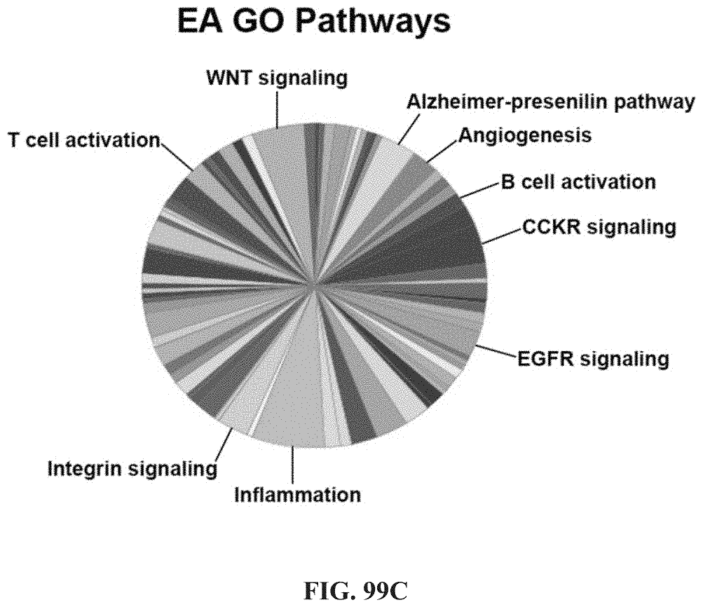

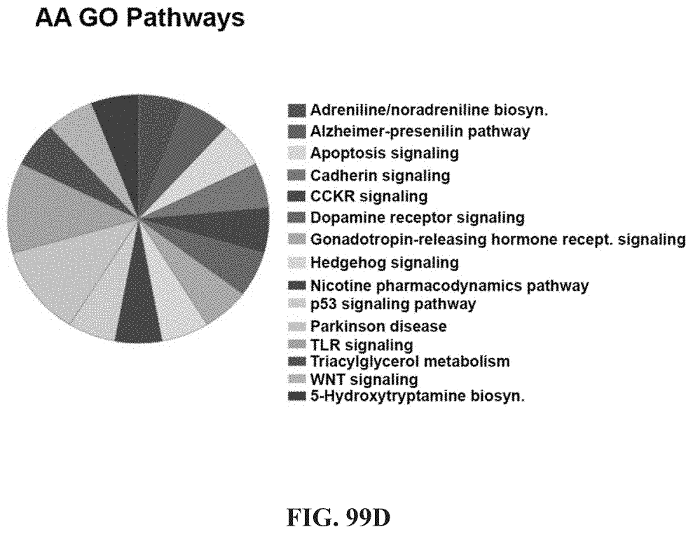

FIGS. 99A-99D show an example of E-Gene functional and pathway analysis, in accordance with disclosed embodiments. PANTHER (v. 13.1) was used to classify EA and AA E-Genes according to gene ontology (GO) biological processes and pathways. The number of EA E-Genes (FIG. 99A) and AA E-Genes (FIG. 99B) assigned to GO biological processes is displayed in each bar graph; GO identifiers are reported to the right of each graph. For pathway analysis, EA E-Gene sequences (FIG. 99C) and AA E-Gene sequences (FIG. 99D) were assigned to GO pathways. EA E-Genes are defined by 78 pathways; several pathways of interest containing 4 or more E-Genes are labeled. AA E-Genes are defined by 15 pathways, as shown in the pie chart.

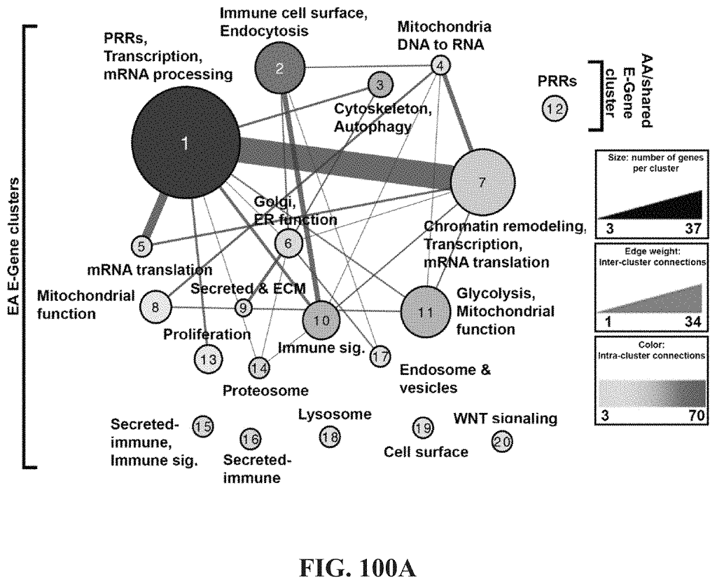

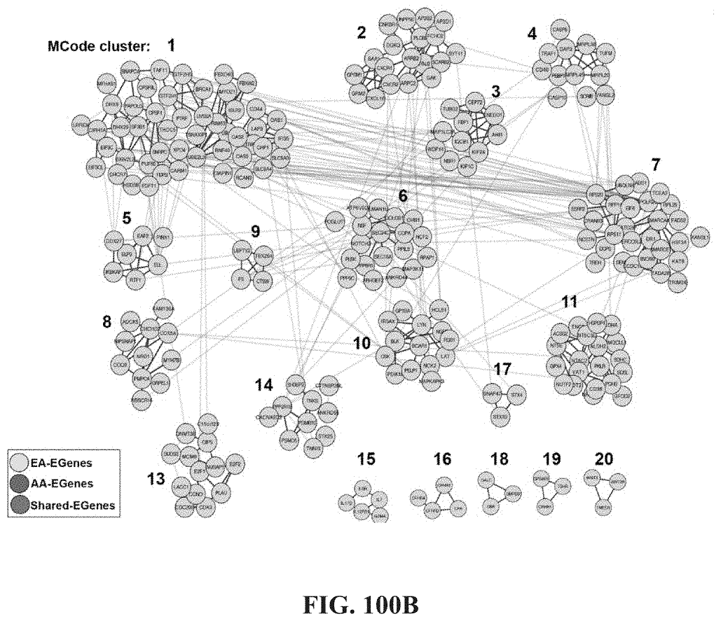

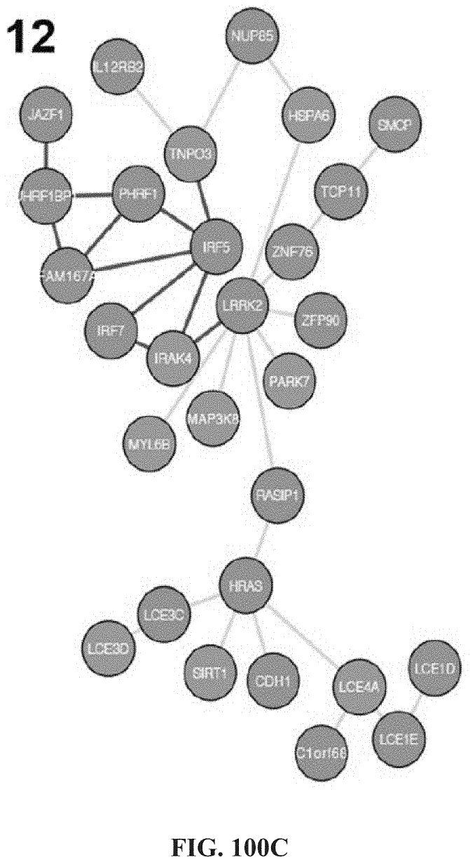

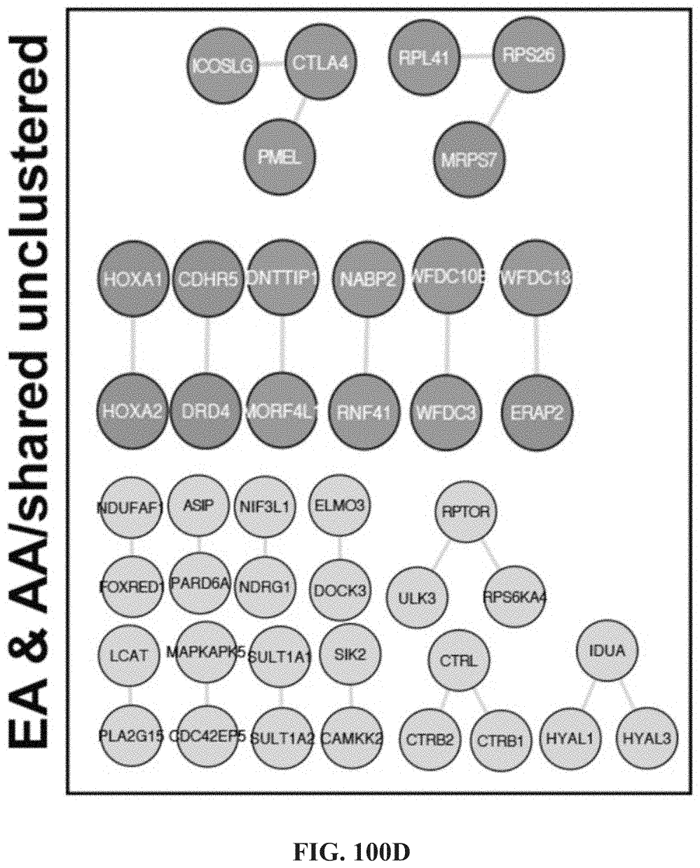

FIGS. 100A-100C show an example of generation of protein-protein interaction (PPI) networks, in accordance with disclosed embodiments. PPI networks and clusters were generated via CytoScape using the STRING and MCODE plugins. Networks were constructed of all EA, AA, and shared (EA+AA) E-Genes. MCODE clusters were determined by the strength of protein-protein interactions, calculated by pooling information from publicly available literature. FIG. 100A shows the cluster metastructure of each network and corresponding BIG-C™ categories, while FIGS. 100B-100C show the specific genes that make up each cluster. FIG. 100D shows EE, AA, and shared (EE+AA) E-Genes that were unclustered.

A set of examples of European-Ancestry (EA) E-Genes are shown in Table 56; a set of examples of African-Ancestry (AA) E-Genes are shown in Table 57; and a set of examples of shared E-Genes (common to both EA and AA) are shown in Table 58.

TABLE 56
European-Ancestry (EA) E-Genes by MCODE Cluster Number

| MCODE | |
|---|---|
| Cluster | |
| Number | Set of European-Ancestry (EA) E-Genes |
| 1 | SNAPC4, TAF11, GTF2H5, MFHAS1, CPSF3L, GTF2H1, BRCA1, DHX8, |
| | PAPOLG, CPSF1, PTRF, UVSSA, LRRIQ4, CIRH1A, DHX29, SF3B1, THOC5, |
| | SNAXIP, EIF3C, SNRPC, XPO4, UBE2L3, EIF3CL, SKIV2L2, PUF60, |
| | CARM1, DHCR7, HSD3B, FDFT1, FDPS, FBXO40, MYOZ1, FBXW2, ISLR2, |
| | CD44, TRIM63, LAP3, OAS1, OAS2, RNF40, CHP1, IFI35, OAS3, SLC9A3, |
| | SLC9A4, CIAPIN1, RCAN3 |
| 2 | CNKSR1, INPP5E, AP3B2, AP3D1, PLCB3, FCHO2, DGKQ, SAA1, ARRB2, |
| | SCARB2, SYT11, CXCR1, CXCR2, GPSM1, GRM2, CXCL16, ARPC2, GAK, |
| | SYNJ2 |
| 3 | CEP72, TUBG2, NEDD1, FBF1, AHI1, MAP1LC3A, IQCB1, KIF24, WDFY4, |
| | NBR1, KIF1C |
| 4 | CASP8, TRAF1, DAP3, MRPL38, TUFM, CD40, PEBP1, MRPL45, MPRL20, |
| | CASP10, SCRIB, VANGL2 |
| 5 | DDX27, EAF2, PINX1, ELP3, ELL, RTF1, IKBKAP |
| 6 | POGLUT1, ATP6V0D, LMAN1L, GOLGB1, CHN1, NSF, SEC24C, COPA, |
| | NCF2, NOTCH2, PPIL3, SEC16A, PLEK, PPP6R1, RPAP1, PPP5C, ARHGEF2, |
| | ANKRD44, MAP3K11 |
| 7 | UBQLN4, ADS1, RPS20, RPP14, EIF6, POLR2, TCEA3, RPL29, ESRP2, |
| | ZRANB3, SMARCA4, FADS2, ATC2IP, RPS11, NCSTN, ERCC6L2, DR1, |
| | H3F3A, KANSL1, DCPS, TREH, SED, CCDC1, INO80, SMARCE1, KAT8, |
| | TADA2B, TRIM24 |
| 8 | FAM136A, ADCK5, CHCHD2, COX5A, NIPSNAP1, NRD1, MYH7B, COQ9, |
| | PMPCA, GRPEL1, WBSCR16 |
| 9 | LEFTY2, TEX264, F5, CTSW |
| 10 | GP1BA, HCLS1, ITGAX, LYN, RGS1, BLK, BCAR1, LAT, CSK, NCK2, |
| | PELP1, MAPKAPK3, PDIK1 |
| 11 | HSPBP1, NT5C3L, ACSS2, NT5E, ALDH2, IMGCLL1, NT5C2, PKLR, SDHC, |
| | SDSL, GPX4, VAT1, PDHB, GFOD2, NUTF2, CD38 |
| 13 | C15orf23, OIP5, DNMT3B, SUDS3, MCM6, E2F1, NUSAP1, E2F2, LACC1, |
| | CCNO, PLAU, CDC20B, CDK3 |
| 14 | SH3BP2, CTTNBP2NL, TNKS, ANKRD65, PP2R1B, PSMB10, STK25, |
| | CACNA2D2, PSMD5, TNNI3 |
| 15 | IL6R, IL7, IL17D, IL12RB1, GZMA |
| 16 | CFHR4, CFHR3, CFHR1, CFH |
| 17 | SNAP47, STX4, STX1B |
| 18 | GALC, SMPD3, GBA |
| 19 | GPBAR1, TSHR, CRHR1 |
| 20 | WNT3, WNT2B, TMED5 |
| Un- | NDUFAF1, FOXRED1, LCAT, PLA2G15, ASIP, PARD6A, MAPKAPK5, |
| clustered | CDC42EP5, NIF3L1, NDRG1, SULT1A1, SULT1A2, ELMO3, DOCK3, SIK2, |
| | CAMKK2, RPTOR, ULK3, RPS6KA4, CTRL, CTRB2, CTRB1, IDUA, HYAL1, |
| | HYAL3 |

TABLE 57
African-Ancestry (AA) E-Genes by MCODE Cluster Number

| MCODE | |
|---|---|
| Cluster | |
| Number | Set of African-Ancestry (AA) E-Genes |
| 12 | TNPO3, HSPA6, IRAK4, PARK7, MYL6B, MAP3K8, LCE3D, SIRT1, LCE4A |
| Un- | ICOSLG, CTLA4, PMEL, RPL41, RPS26, DNTTIP1, MORF4L1, NABP2, |
| clustered | RNF41, WFDC10B, WFDC3, WFDC13 |

TABLE 58
Shared E-Genes (common to both EA and AA) by MCODE Cluster Number

| MCODE | |
|---|---|
| Cluster | |
| Number | Set of Shared E-Genes |
| 12 | NUP85, IL12RB2, JAZF1, UHRF1BP, PHRF1, FAM167A, IRF7, IRF5, LRRK2, |
| | ZNF76, TCP11, SMCP, ZFP90, RASIP1, HRAS, LCE3C, CDH1, C1orf68, |
| | LCE1E, LCE1D |
| Un- | MRPS7, HOXA1, HOXA2, CDHR5, DRD4, ERAP2 |
| clustered | |

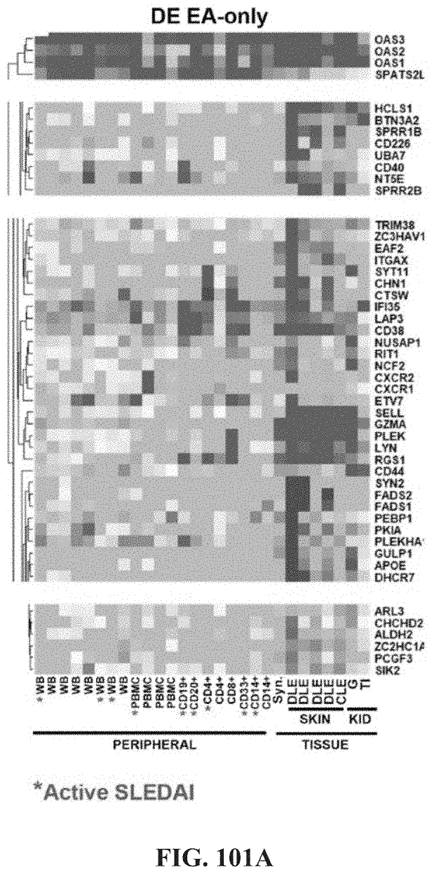

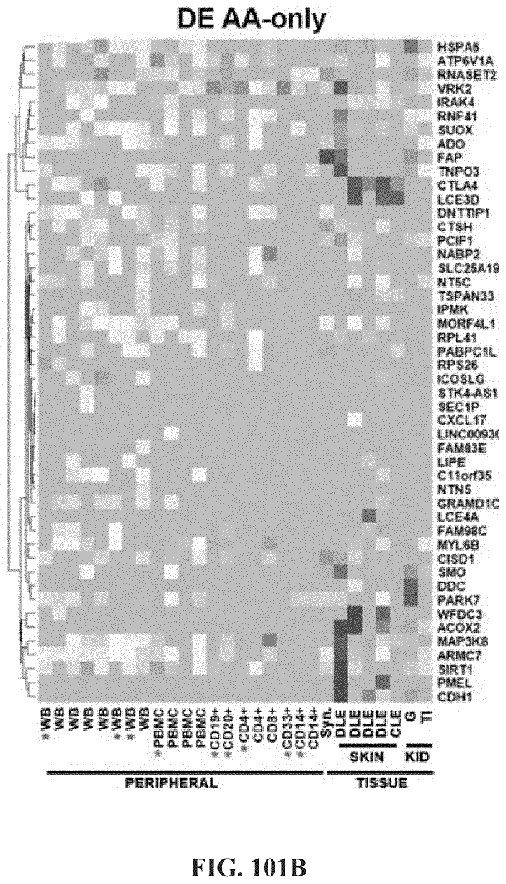

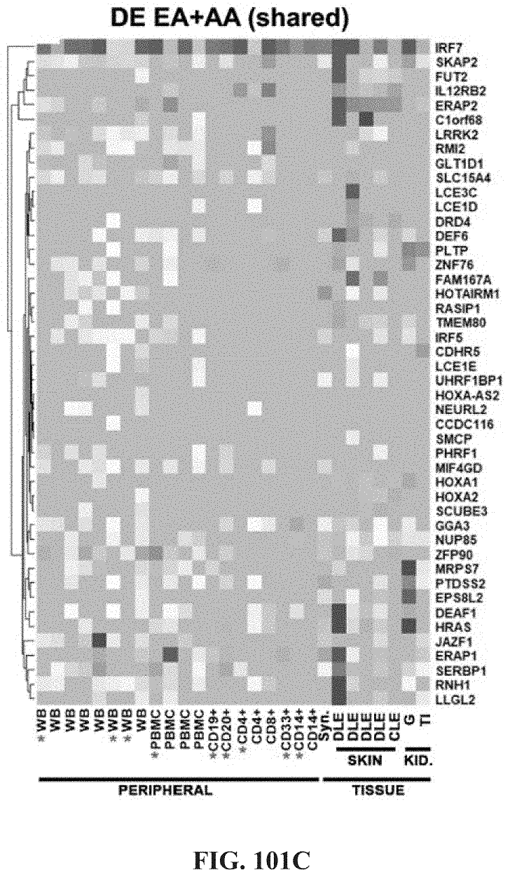

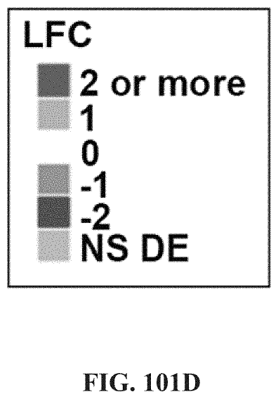

FIGS. 101A-101D show an example of a comparison of E-Genes predicted from SLE-associated SNPs with SLE differential expression datasets, in accordance with disclosed embodiments. Predicted E-Genes were matched with SLE differential expression (DE) data and organized by ancestry. FIG. 101A shows the fold-change variation of EA-only E-Genes. Due to the large number of differentially expressed (DE) EA E-Genes, a selection of the most highly upregulated and downregulated genes are presented. FIG. 101B shows AA-only DE E-Genes, and FIG. 101C shows DE E-Genes common to both the AA and EA gene sets. Color for all three heatmaps represents log fold change, as indicated by the legend underneath the central heatmap (FIG. 101D). Red asterisks indicate active SLEDAI datasets.

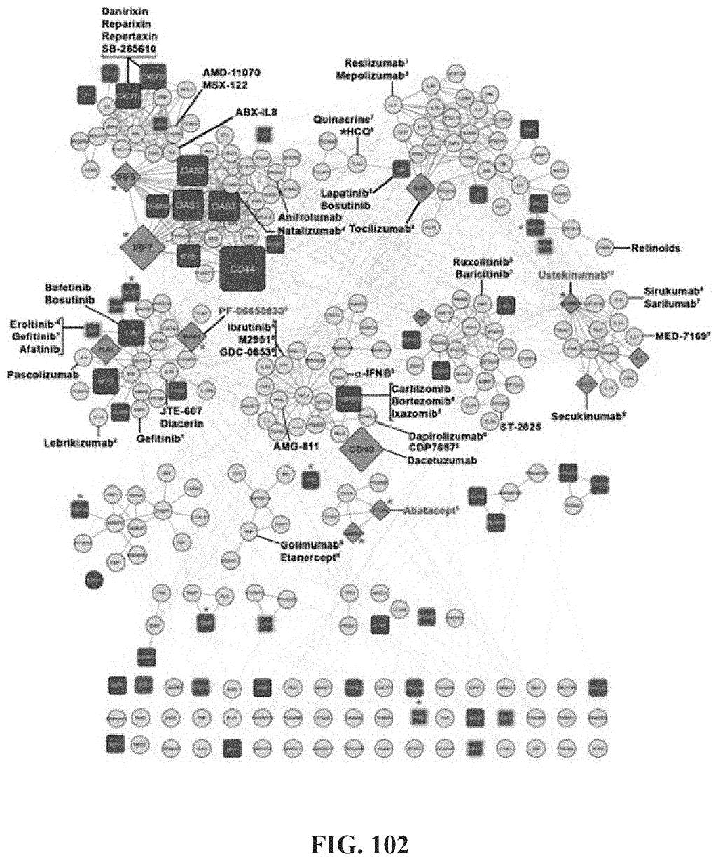

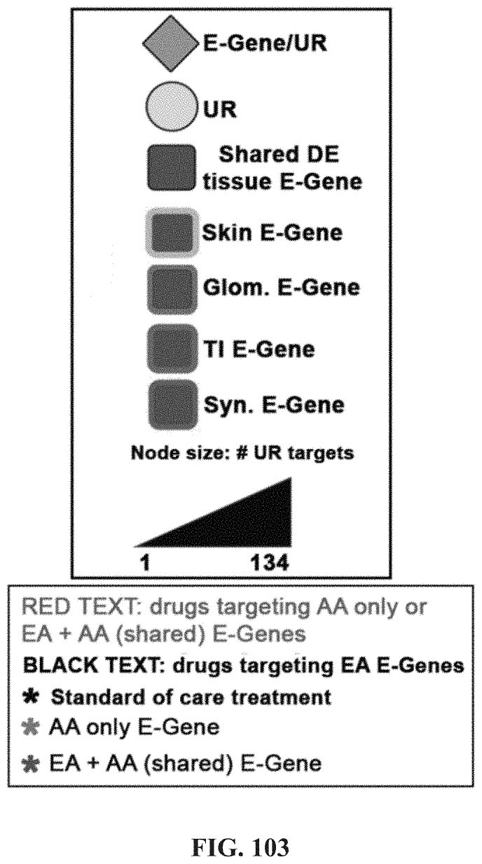

FIGS. 102-103 show an example of a comparison of E-Genes predicted from SLE-associated SNPs with SLE differential expression datasets, in accordance with disclosed embodiments. Compounds targeting EA, AA, shared tissue E-Genes and associated pathways are shown. Differentially expressed E-Genes from synovium, skin, and kidney tissue datasets were first compared to immune-specific gene lists. Overlapping genes were used as input for IPA upstream regulator analysis. PPI networks and clusters were generated via CytoScape using the STRING and MCODE plugins. MCODE clusters were determined by the strength of protein-protein interactions, calculated by pooling information from publicly available literature. Select drugs acting on targets are shown. Where available, CoLT scores (−16 to +11) are depicted in superscript.

This multi-level combined genetic and genomic bioinformatics analysis is capable of defining gene regulatory pathways which not only reflect differences in EA and AA populations, but also represent candidate pathways that may be the target of ancestry-specific therapies. Ancestral SNP-associated E-Genes and gene expression profiles illustrate fundamental differences in lupus molecular pathways between ancestral groups. In particular, different or unique sets of drugs may be particularly effective at treating lupus within each ancestral group based on these differences in lupus molecular pathways.

### Example 32: Selective Histone Deacetylase 6 (HDAC6) Inhibition Normalizes B Cell Activation and Germinal Center Formation in a Model of Systemic Lupus Erythematosus (SLE)

Autoantibody production by plasma cells (PCs) may play a pivotal role in the pathogenesis of systemic lupus erythematosus (SLE). The molecular pathways by which B cells become pathogenic PC secreting autoantibodies in SLE are incompletely characterized. Histone deactylase 6 (HDAC6) refers to a unique cytoplasmic HDAC that modifies the interaction of a number of tubulin-associated proteins. Inhibition of HDAC6 may be shown to be beneficial in murine models of SLE; however, the downstream pathways accounting for the therapeutic benefit may not be clearly delineated (e.g., in human subjects). Experiments were conducted to demonstrate that selective HDAC6 inhibition effectively abrogates abnormal B cell activation in SLE. A set of NZB/W lupus mice were treated with the selective HDAC6 inhibitor, ACY-738, for four weeks beginning at 20 weeks of age. After only 4 weeks of treatment, manifestation of lupus nephritis (LN) was observed to be greatly reduced in these animals. Next, RNAseq was performed to determine the genomic signatures of splenocytes from treated and untreated mice, and computational cellular and pathway analyses were performed to reveal multiple signaling events associated with B cell activation and differentiation in SLE that were modulated by HDAC6 inhibition. PC development was observed to be abrogated, and germinal center (GC) formation was observed to be greatly reduced. When the HDAC6 inhibitor-treated lupus mouse gene signatures were compared to human lupus patient gene signatures, the results showed numerous immune and inflammatory pathways increased in active human lupus were significantly decreased in the HDAC6 inhibitor-treated animals. Pathway analysis showed that alterations in cellular metabolism may contribute to the normalization of lupus mouse spleen genomic signatures, and this was confirmed by direct measurement of the impact of the HDAC6 inhibitor on metabolic activities of murine spleen cells. Taken together, these results show that HDAC6 inhibition decreases B cell activation signaling pathways and reduces PC differentiation in SLE. Further, these results show that a critical event of HDAC6 inhibition may be modulation of cellular metabolism.

Introduction

Systemic lupus erythematosus (SLE) is a multi-organ autoimmune disease characterized by the production of pathogenic antibodies with the formation of immune complexes that may be deposited in various tissues. Plasma cells (PCs) are differentiated B cells that may be responsible for the production of antibodies that provide defense from invading foreign pathogens. After activation, B cells may either (a) form short-lived extrafollicular plasmablasts that are critical for early protective immunity, or (b) enter specialized regions of secondary lymphoid tissue that facilitate T cell:B cell collaboration—either germinal centers (GCs) or extra-follicular foci—and undergo extensive proliferation, eventually becoming PC that produce high avidity antibody via somatic hypermutation. In lupus, PCs differentiated from active B cells may produce autoantibodies, such as anti-dsDNA and anti-RNA-binding proteins, which bind self-antigens to form immune complex that deposit in blood vessels and renal glomeruli, leading to vasculitis and nephritis. Many details of the intracellular event regulating the process and regulation of T cell:B cell collaboration and PC generation in SLE may not yet have been delineated.

Post-translational modification (PTM) of proteins may be an important approach to regulate protein:protein interactions and downstream cellular functions. In SLE, PTM-modified self-proteins may play important roles in induction and initiation of autoimmune response by creating neo-epitopes. The isotype of autoantibodies may be determined by the modified histone proteins in murine and human lupus. Among the various PTMs of proteins, acetylation may play a major role. Further, SLE may involve significant enrichment of lysine acetylation proteins, which widely contribute to a variety of cellular functions. Acetylation/deacetylation events are reversible PTM on lysine residues of histone and non-histone proteins, and may be essential for specific protein:protein interactions and in the nucleus for gene regulation. These reactions are typically catalyzed by enzymes with histone acetyltransferase (HAT) or histone deacetylase (HDAC) activity. HDACs may be classified into four subclasses: three Zn²⁺-dependent classes (I, II, and IV), and one NADtdependent class III. Class II may be subdivided into class IIa and class IIb. HDAC6 may belong to HDAC class Bb and is largely cytoplasmic in location. It may be associated with non-histone substrates, including a-tubulin, heat shock protein 90 (HSP90), cortactin, and others, and may modulate immune cell function in various ways, including modifying BCL6 function and B cell maturation.

In some cases, selective HDAC6 inhibitor ACY-738 administered to pre-disease lupus-prone NZB/W mice prevent the onset of lupus nephritis (LN). NZB/W mice were treated for only four weeks after disease onset, and mechanisms by which this cytoplasmic HDAC inhibitor may alter the cellular functions involved in lupus pathogenesis, especially the maintenance of GC and PC generation, were determined. To accomplish this, changes were assessed in the mRNA transcriptome mediated by selective HDAC6 inhibition using RNA-Sequencing (RNA-seq) analysis of whole splenocytes. Results indicated that HDAC6 inhibition in NZB/W mice led to global changes in gene expression. Results also showed that, phenotypically, decreased glomerulonephritis was coupled with reduced IgG and C3 deposition and decreased GC and PC populations. Furthermore, reduced B cell activation was observed following HDAC6 inhibitor treatment, and underlying this was a change in cellular metabolism. Taken together, these data indicate that targeting autoreactive B cells through increased acetylation may limit cell activation and differentiation in lupus, thereby providing therapeutic benefit.

Materials and Methods

ACY-738 treatment of mice in a murine disease model was performed as follows. Female New Zealand Black/White F1 (NZB/WF1/J) (NZB/W) mice were obtained from The Jackson Laboratory (Bar Harbor, Me., USA). For ACY-738 treatment, NZB/W mice were given a diet mixed with or without 200 mg/kg ACY-738, which was purchased from Envigo (form 8640, Huntingdon, UK). Treatment started at 20 weeks of age, when the animals began to show signs of mild proteinuria (30 mg/dL by dipstick analysis). All animals were allowed food and water ad libitum. Treatment was continued for four weeks, at which time the animals were euthanized.

Immunofluorescence was performed as follows. At the termination of the experiment, the spleens and kidneys of the mice were removed. One portion of the spleen and the kidney was embedded in Tissue-TekVR optimal cutting temperature compound (O.C.T.™) (Sakura Finetek, Torrance, Calif., USA), and frozen rapidly in a freezing bath of dry ice and 2-methylbutane. Frozen OCT samples were cryosectioned into 5-μm and 10-μm sections, respectively. Frozen slides were warmed to room temperature and allowed to dry for 30 minutes, followed by fixation in cold acetone at room temperature for 10 minutes. After washing in PBS, slides were blocked with PBS containing 1% bovine serum albumin (BSA) and anti-mouse CD16/32 for 20 minutes at room temperature. Slides were then incubated with a fluorochrome-conjugated antibody mixture for 1 hour at room temperature in a dark humid box. Slides were mounted with Prolong Gold containing DAPI (Life Technologies, Carlsbad, Calif., USA). The following anti-mouse antibodies were used in immunohistochemical analysis: anti-IgG-phycoerythrin (PE) (eBioscience, Santa Clara, Calif., USA), anti-C3-fluorescein isothiocyanate (FITC) (Cedarlanelabs, Burlington, Canada), anti-IgD-phycoerythrin (PE) (eBioscience, Santa Clara, Calif., USA), anti-CD3-APC (Biolegend, San Diego, Calif., USA), Peanut Agglutinin (PNA)-fluorescein isothiocyanate (FITC) (Burlingame, Calif., USA), anti-CD138− phycoerythrin (PE) (eBioscience, Santa Clara, Calif., USA) and anti-IgM-V450 (BD bioscience, Franklin Lakes, N.J.). Slides stained with antibodies were read and visualized using an EVOSVR FL microscope (Advanced Microscopy Group, Grand Island, N.Y., USA) with 40× and 20× objectives for kidney and for spleen, respectively. Six randomly selected glomeruli from each sample were pictured and then analyzed by using ImageJ software (National Institutes of Health, Rockville, Md., USA) to calculate the deposition of IgG and C3. For spleens, a total of 20 spots were imaged for each group of 4 mice, with five random spots imaged from each mouse, from which representative figures were selected.

The mRNA isolation and sequencing were performed as follows. Total RNA was isolated from whole splenocytes using the miRNeasy Mini Kit (Qiagen, Germantown, Md., USA) per manufacturer's instructions. To remove residual amounts of DNA contamination in isolated RNA, on-column DNase digestion with RNase-Free DNase was performed. The RNA concentration was quantified using a NanoDrop 2000 system. Total RNA was sent to Beckman Coulter (Danvers, Mass., USA) for 2×100 bp paired-end Illumina RNA sequencing with an average of 40 million reads per sample. Sequencing data (FASTQ files) was trimmed for both adaptor sequences and quality using a combination of ea-utils and Btrim. Sequencing reads were then aligned to the genome (Ensembl.org 38.74) using Bowtie2/Tophat2 and counted via HTSeq.

Gene set variation analysis (GSVA) was performed as follows. The open source GSVA (V1.25.0) software package for R/Bioconductor was used as a non-parametric, unsupervised method for estimating the variation of pre-defined gene sets in patient and control samples of microarray expression data sets. Raw RNAseq counts transformed into log 2 expression values for pre-defined gene sets were used as the inputs for GSVA. Enrichment scores (GSVA scores) were calculated non-parametrically using a Kolmogorov Smirnoff (KS)-like random walk statistic; a negative value for a particular sample and gene set indicated that the gene set has a lower expression than the same gene set in a sample with a positive value. The enrichment scores (ES) were the largest positive and negative random walk deviations from zero, respectively, for a particular sample and gene set. The positive and negative ES for a particular gene set depend on the expression levels of the genes that form the pre-defined gene set. The increased transcripts for SLE plasma cells (PC) (e.g., as described by Lugar et al., “Molecular characterization of circulating plasma cells in patients with active systemic lupus erythematosus,” PLoS One, 7(9), p. e44362, 2012; which is incorporated herein by reference in its entirety) were used to determine the enrichment of PC. Tfh cells were determined by expression of Bcl6, Pdcd1, Icos, Ascl2, and Tnfsf4. Markers of germinal centers were determined by expression of Gcsam, Nuggc, Rgs13, Klhl6, Aicda, Bcl6, and Irf4.

I-Scope analysis was performed as follows. I-scope is a tool used to identify immune infiltrates in gene expression datasets. I-scope was created through an iterative search of more than 17,000 genes identified in more than 50 microarray datasets. From this search, a set of 1,226 candidate genes was identified and researched for restriction in hematopoietic cells, as determined by the HPA, GTEx, and FANTOMS datasets (proteinatlas.org); 926 genes met the criteria for being mainly restricted to hematopoietic lineages (brain, reproductive organs exclusions). These genes were researched for immune cell specific expression in 30 hematopoietic sub-categories: T cells, regulatory T cells, activated T cells, anergic cells, CD4 T cells, CD8 T cells, gamma-delta T cells, NK/NKT cells, T & B cells, B cells, activated B cells, T &B & monocytes, monocytes & B cells, MHC Class II expressing cells, monocyte dendritic cells, dendritic cells, plasmacytoid dendritic cells, Langerhans cells, myeloid cells, plasma cells, erythrocytes, neutrophils, low density granulocytes, granulocytes, platelets, and all hematopoietic stem cells. Transcripts are entered into I-scope, and the number of transcripts in each category is calculated and represents the specific immune cell populations in each dataset.

Pathway analyses were performed as follows. Ingenuity Pathway Analysis (IPA) software (Qiagen, Venlo, Netherlands) was used to calculate Z scores based on increased and decreased transcript levels in HDAC6 inhibitor samples compared with transcript levels in controls). Z scores≥2 or ≤−2 and overlap p values≤0.05 were considered significant. IPA scores were used to determine whether pathways were up-regulated or repressed based on whether transcripts were increased or decreased relative to controls in the entry dataset.

Gene ontology (GO) biological pathway (BP) analysis was performed as follows. Increased and decreased transcripts were annotated with GO BP terms separately, and overlap p values were determined. Pathways were considered enriched or reduced if they had associated p values less than 0.01.

Biologically informed gene clustering (BIG-C) analysis was performed as follows. BIG-C is a custom functional clustering tool developed to annotate the biological meaning of large lists of genes. Separately, increased and decreased genes are sorted into 52 categories based on their most likely biological function and/or cellular localization based on information from multiple online tools and databases including UniProtKB/Swiss-Prot, GO Terms, MGI database, KEGG pathways, NCBI PubMed, and the Interactome. Each gene is placed into only one category based on its most likely function to eliminate the redundancy in enrichment sometimes found in GO BP annotation.

A comparison of HDAC6 inhibitor-treated NZB/W RNA seq to human SLE tissue microarray data was performed as follows. The comparison analysis feature of IPA was used to compare the Z scores between processed microarray data from differential expression (DE) analysis of four human SLE tissue experiments and the DE analysis of the HDAC6 inhibitor-treated versus untreated NZB/W mice. Raw data from lupus tissue datasets were obtained from the GEO repository: GSE36700 for lupus synovium (4 OA, 4 SLE patients), GSE72535 for discoid lupus skin (8 healthy control (HC), 9 DLE), GSE32591 for LN dissected glomerulus WHO class 3 or 4 (32 HC, 22 SLE) and GSE32591 for lupus nephritis dissected tubulointerstitium from WHO CLASS 3 or 4 LN (32 HC, 22 SLE). Differential Gene Expression (DE) was carried out for each dataset of SLE tissue samples and controls. GCRMA normalized expression values were variance corrected using local empirical Bayesian shrinkage, before calculation of DE using the ebayes function in the open source BioConductor LIMMA package (bioconductor.org/packages/release/bioc/html/limma.html). Resulting p-values were adjusted for multiple hypothesis testing and filtered to retain DE probes with a false detection rate (FDR) less than 0.05.

Metabolic enzyme function studies were performed as follows. Citrate synthase (CS) may catalyze the formation of citrate and coenzyme A (CoASH) from acetyl-CoA and oxaloacetate. CoASH may reduce DTNB, and CS activity was determined from the reduction of DTMB over time. Briefly, at sacrifice, splenocytes from ACY-738 treated mice and untreated control mice were lysed (10⁶ cells/200 μL) in a buffer containing 0.1% Triton X-100, 1 mM EDTA, 50 mM Tris, pH 7.4, and Protease Inhibitor Cocktail (Nacalai Tesque). The CS assay was carried out using 20 μL of the lysates in 96-well plates. CS activity was measured by adding 80 μL of the reaction solution containing 0.1 mM DTNB, 0.3 mM acetyl-CoA, 1 mM oxaloacetate, and 50 mM Tris at pH 7.4, to each well. Absorbance was measured on a spectrophotometer (BioTek Synergy 2, Winooski Vt., USA) at 405 nm at 37° C. every 12 seconds for 5 minutes. Total protein concentration of the lysates was quantified by a Bio-Rad Protein Assay, and CS activity was normalized to the total protein concentration. CS activity was calculated as the rate of increase of absorbance with time. All samples were run in triplicate. Maximum activity was calculated and reported as μM per mg per minute.

For the determination of β-hydroxyacyl-CoA dehydrogenase activity, the oxidation of NADH to NAD was measured. In this procedure, splenocytes were added to 190 μL of a buffer containing 0.1M liquid triethanolamine, 5 mM EDTA tetrasodium salt dihydrate, and 0.45 mM NADH. Following a 2-minute background reading, 15 μL of 2 mM acetoacetyl CoA was added to initiate the reaction. Absorbance was measured at 340 nm every 12 seconds for 5 minutes at 37° C. Maximum activity was calculated and reported as μM per mg per minute.

Cytochrome c oxidase, which transfers electrons between complex III and IV of the electron transport chain, was assayed based on the oxidation of ferrocytochrome c to ferricytochrome c by cytochrome c oxidase. Horse heart cytochrome c (Sigma Aldrich, 2 mg/mL) was dissolved in a 10-mM potassium phosphate buffer containing 10 mg/mL of sodium dithionite. 10 uL of splenocyte extracts were added to 290 uL of the reduced cytochrome c test solution. The rate of cytochrome C oxidation was measured spectrophotometrically as a reduction in absorbance at 550 nm every 10 seconds for 5 minutes at 37° C. Maximum cytochrome c oxidase activity was expressed relative to protein content and reported as mol/mg/min.

Fatty acid and glucose oxidation studies were performed as follows. Splenocytes were isolated from spleens from eight-week-old NZB/W female mice. T cells and B cells were enriched from splenocytes using negative selection with a magnetic-activated cell sorting kit (Miltenyi Biotec, Auburn, Calif., USA). Cells were seeded in a 24-well flat-bottomed plate at a density of 10⁶ cells/mL in 1 mL RPMI-1640 (HyClone, South Logan, Utah, USA) supplemented with 1 mm sodium pyruvate, 2 mM L-glutamine, 100 U/mL penicillin, 100 μg/mL streptomycin (HyClone), 5.5×10⁻² mM 2-mercaptoethanol (Gibco BRL Life Technologies, Paisley, UK) and 10% heat-inactivated bovine calf serum (HyClone) per well. For T cells stimulation, plates were pre-coated with anti-CD3 (Invitrogen), and T cells were stimulated with anti-CD28 (Invitrogen) with or without the addition of 4 μM ACY-738 (treatment) or DMSO (control) followed by 24 hours incubation at 37° C. with 5% CO₂. B cells were cultured with Lipopolysaccharide (LPS: Escherichia coli serotype 0111:B4; Sigma-Aldrich, St Louis, Mo.) (50 μg/mL) and treated with ACY-738 (4 μM) or DMSO (control) for 24 hours, after which the cells were collected and metabolism analysis was performed. Substrate metabolism was assessed. Briefly, fatty acid oxidation was measured using radiolabeled fatty acid ([1-¹⁴C]-palmitic acid, American Radiolabeled Chemicals, St. Louis, Mo.) to quantify ¹⁴CO₂ production from the oxidation of isolated B and T cells. Cells were incubated in 0.5 μCi/mL of [1-¹⁴C]-palmitic acid for 1 hour, after which the media was acidified with 200 μL 45% perchloric acid for 1 hour to liberate ¹⁴CO₂. The ¹⁴CO₂ was trapped in a tube containing 1 M NaOH, which was then placed into a scintillation vial with 5 mL scintillation fluid. The vial's ¹⁴C concentrations were measured on a 4500 Beckman Coulter scintillation counter. Glucose oxidation was assessed in the same manner as fatty acid oxidation, with the exception that [U-¹⁴C] glucose was substituted for [1-¹⁴C]-palmitic acid. Oxidation values were normalized to total protein content, as assessed via a commercially available bicinchoninic acid (bca) procedure (Thermo Fisher Scientific, Waltham, Mass., USA) and expressed as nM per mg protein per hour.

The code and data were obtained was follows. The R bioconductor packages limma and Gene set variation analysis (GSVA) are open source code available at www.bioconductor.org. The statistical analysis included analyzing data by student t test with GraphPad Prism software. Statistically significant differences are followed by * (P<0.05), ** (P<0.01), *** (P<0.001), and **** (P<0.0001).

Study approval was obtained as follows. The animal experiments followed the requirement of the Institutional Animal Care and Use Committee (IACUC) at Virginia Tech, Va., USA, and maintained under specific pathogen-free conditions at Virginia Tech College of Veterinary Medicine. All of operations of animals were in compliance with the Guide for the Care and Use of Laboratory Animals.

Results

ACY-738 is a hydroxamic acid HDAC6 inhibitor that is highly selective for HDAC6. ACY-738 inhibits HDAC6 with a high potency, and with substantially greater potency than it inhibits HDAC1 (the next most affected target). It is known that ACY-738 is selective for HDAC6 inhibition. ACY-738 has been shown to induce tubulin acetylation (a marker of HDAC6 inhibition) at concentrations where histone acetylation (a marker of Class 1 HDAC inhibition) is minimal, indicating that the inhibition was primarily cytosolic. Further, it has been shown that 100 mg/kg/day of ACY-738 in rodent chow achieves an estimated plasma concentration of 100 nM (e.g., Jochems, J., et al., 2014, Antidepressant-like properties of novel HDAC6-selective inhibitors with improved brain bioavailability, Neuropsychopharmacology 39(2): 389-400; which is incorporated herein by reference in its entirety).

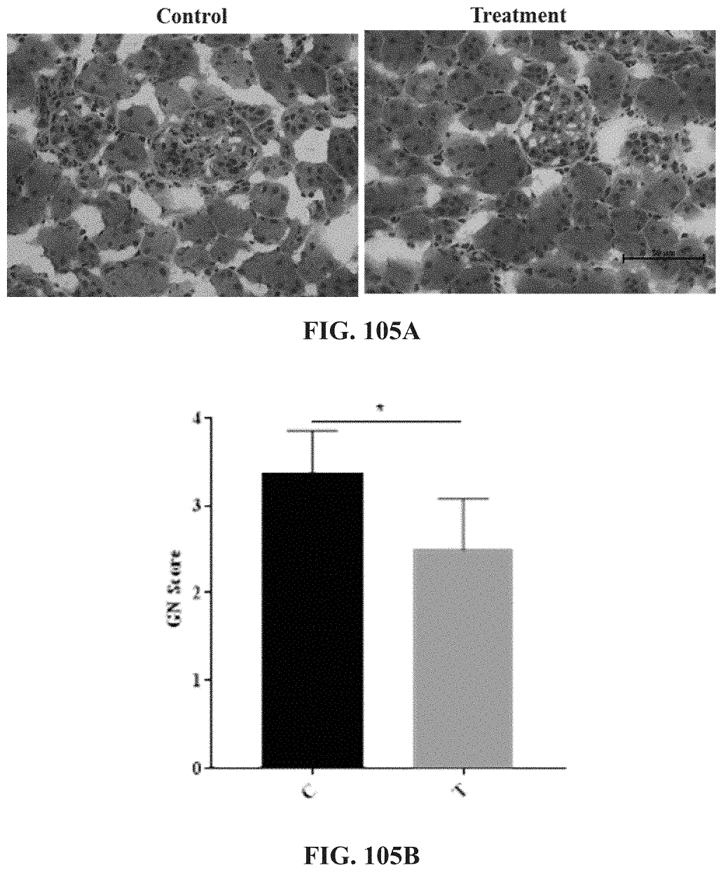

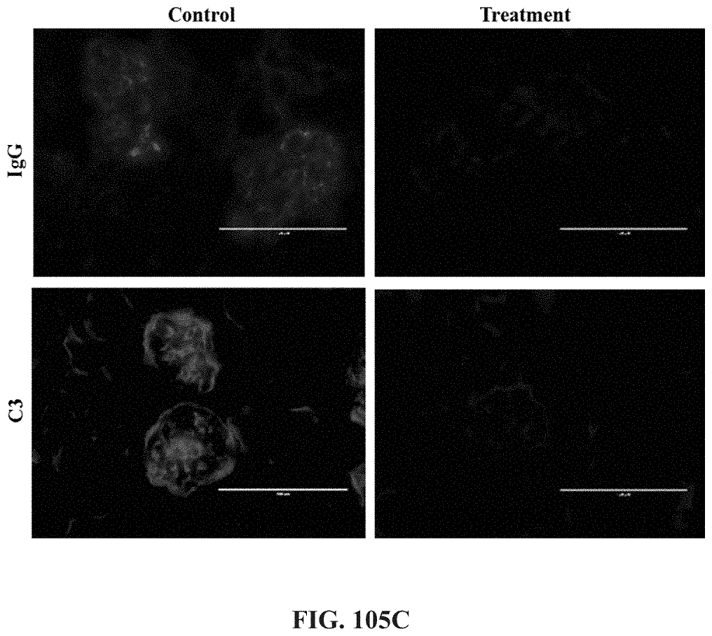

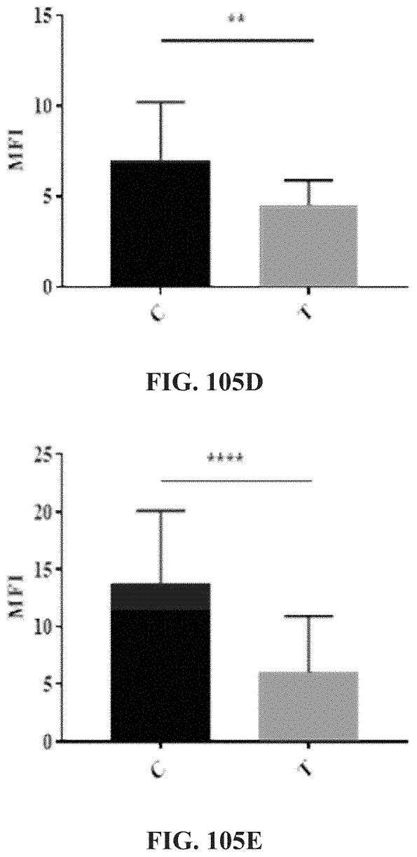

Our results showed that inhibition of HDAC6 improves established lupus nephritis (LN). To simulate the therapeutic paradigm in human lupus, 20-week-old NZB/W F1 female (NZB/W) mice with established LN were treated with the selective HDAC6 inhibitor ACY-738. After only 4 weeks, ACY-738-treated mice exhibited significantly less renal pathology than the untreated group (FIGS. 105A-105B). Moreover, the deposition of IgG and C3 in glomeruli, which may contribute to the progression of renal inflammation, was significantly decreased in the ACY-738 treated group compared to the untreated control group (FIGS. 105C-105E).

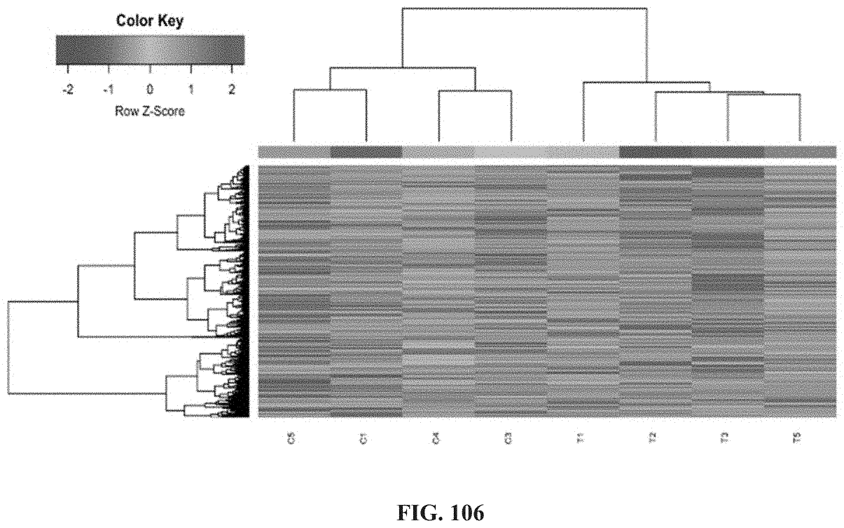

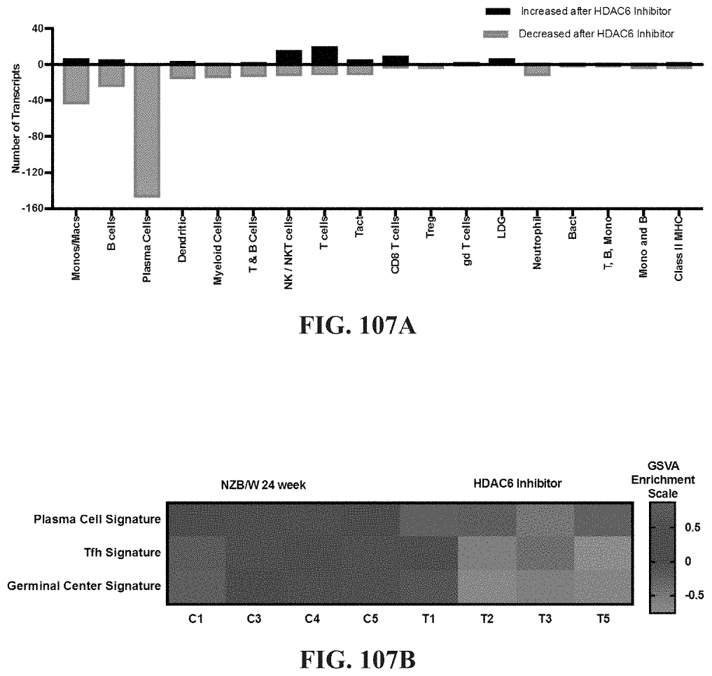

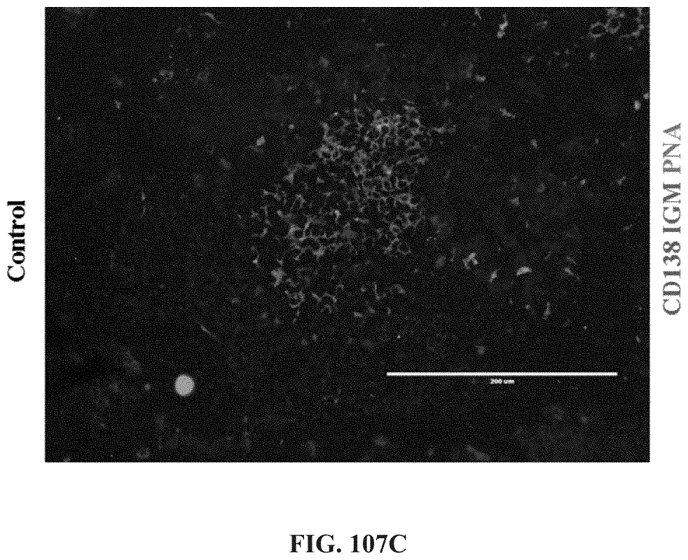

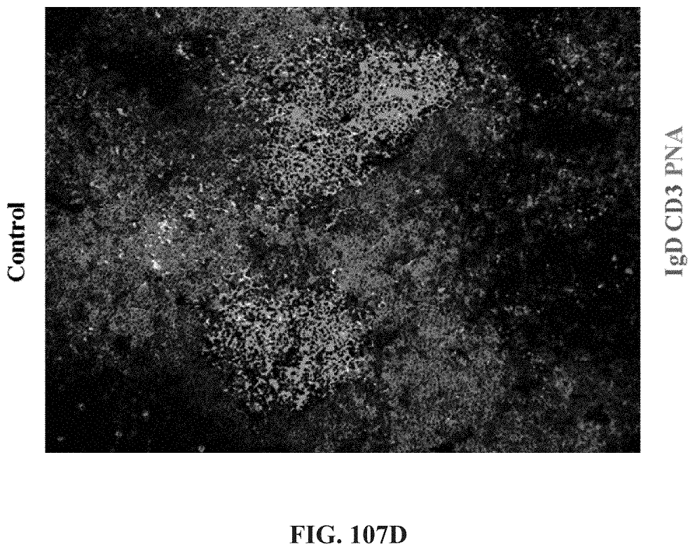

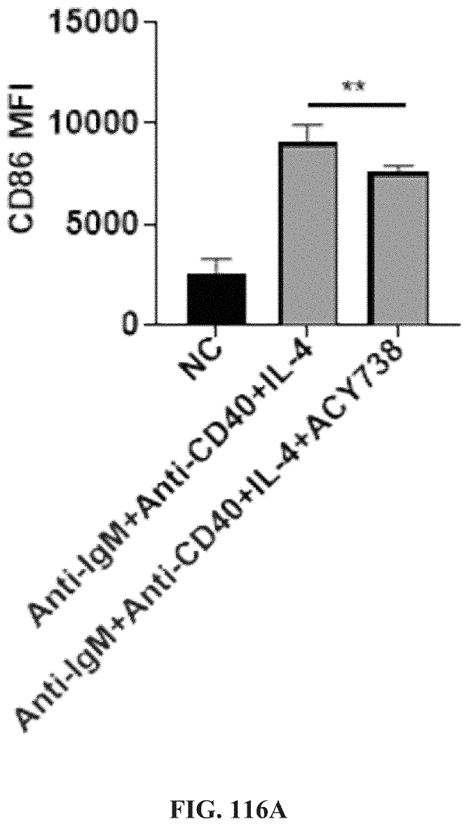

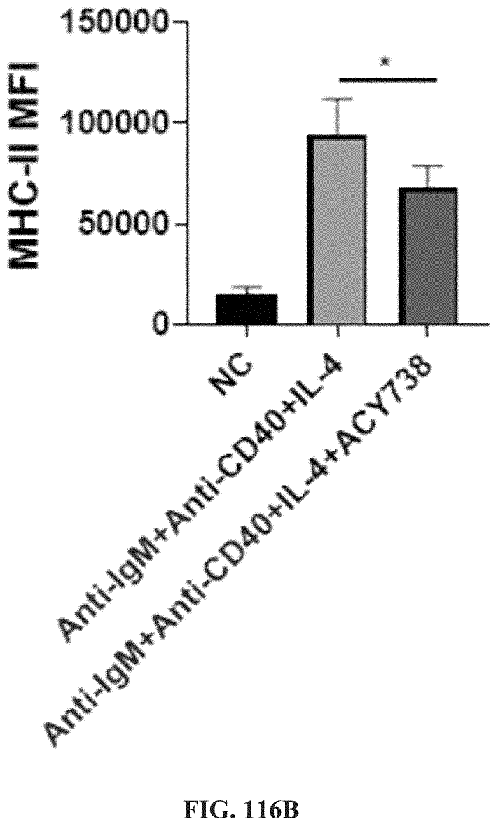

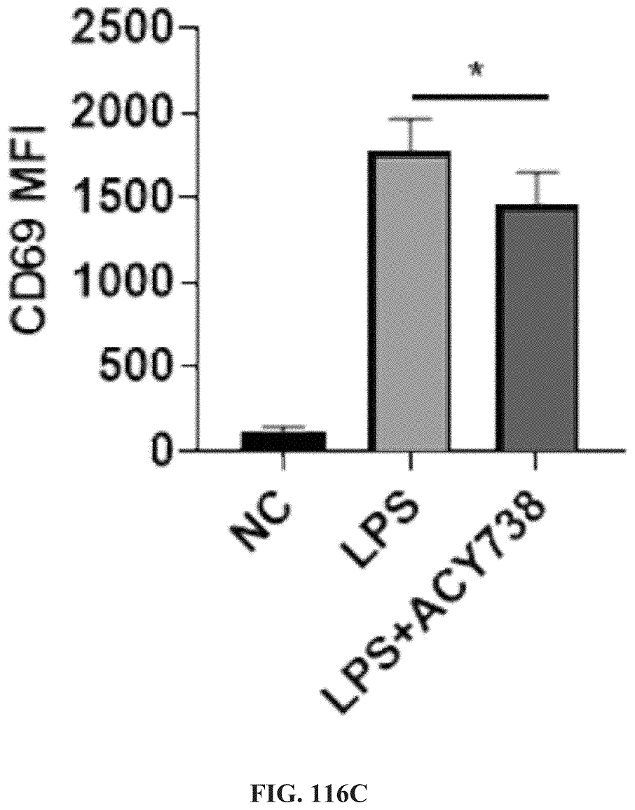

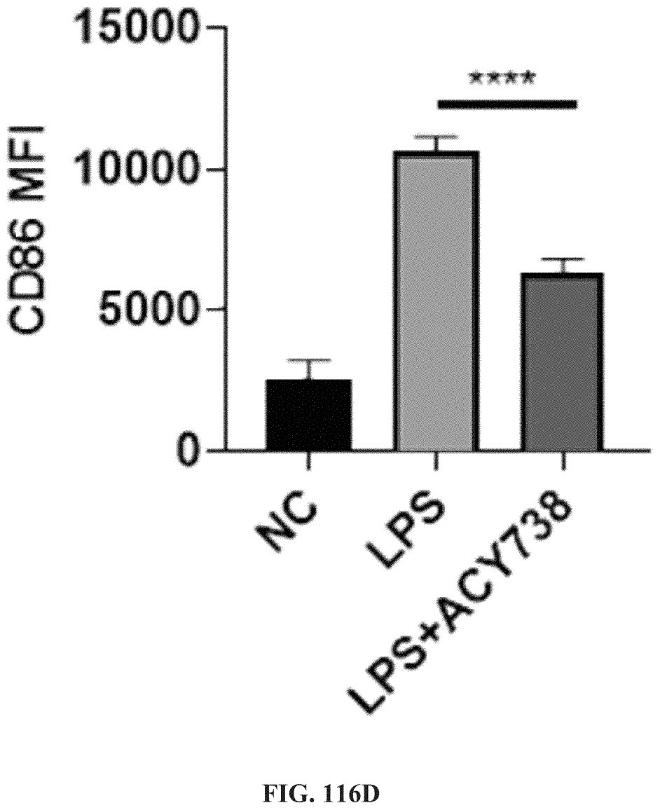

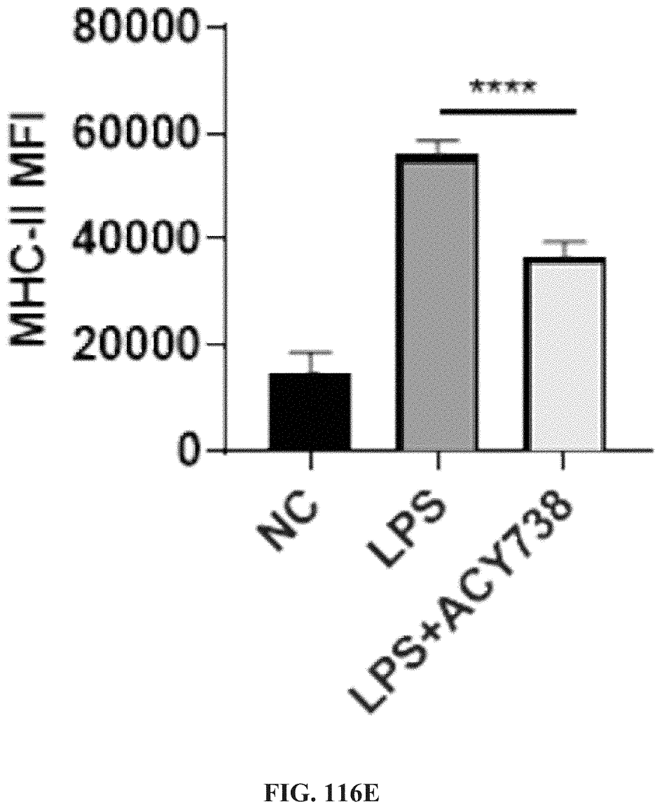

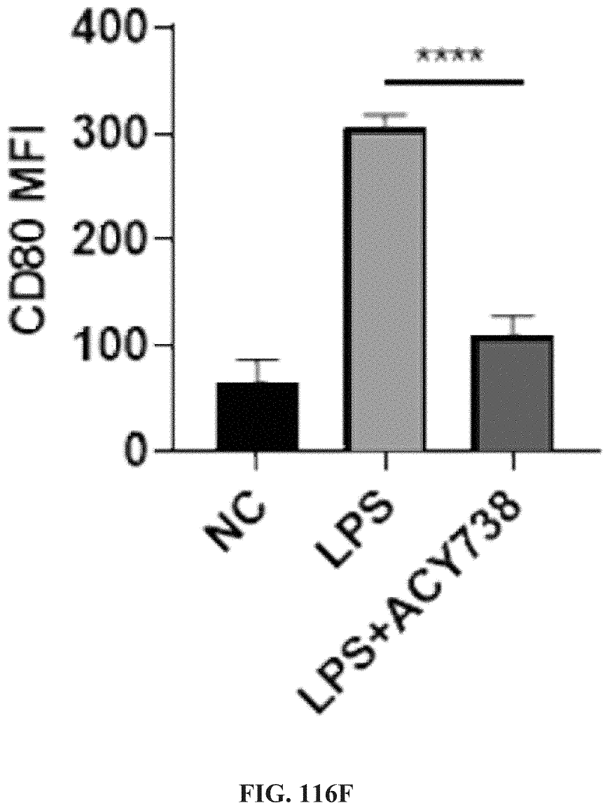

Further, the results showed suppression of B cell responses by HDAC6 inhibitor. To investigate the mechanisms of HDAC6 inhibition on autoimmune responses, changes were analyzed in splenic composition by carrying out bulk RNA sequencing on total splenocytes from ACY-738-treated mice and untreated control NZB/W mice (FIG. 106). Analysis of global gene expression changes by hierarchical clustering showed that 3,911 transcripts were differentially expressed between the treated and untreated samples. Among these, 1,922 genes were up-regulated, and 1,989 genes were down-regulated in the ACY-738-treated group as compared to the untreated control group. To determine whether HDAC6 inhibition led to changes in cell populations in the spleen of treated mice, the I-Scope clustering program was employed to identify immune and inflammatory cell types based on gene expression. Control experiments were performed to demonstrate the specificity and lack of cross reactivity of I-scope (FIGS. 116A-116F). Results showed that HDAC6 inhibition led to a profound decrease in transcripts associated with plasma cells, B cells and inflammatory myeloid cells (FIG. 107A and Tables 58-59) as well as more modest decreases in other immune/inflammatory cells.

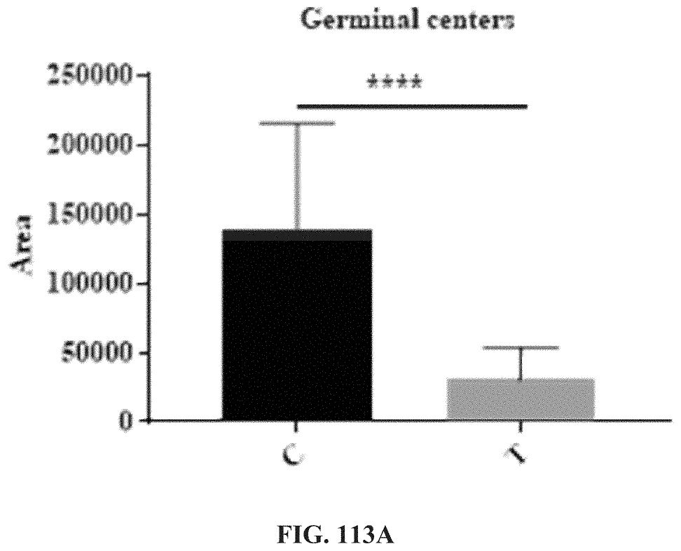

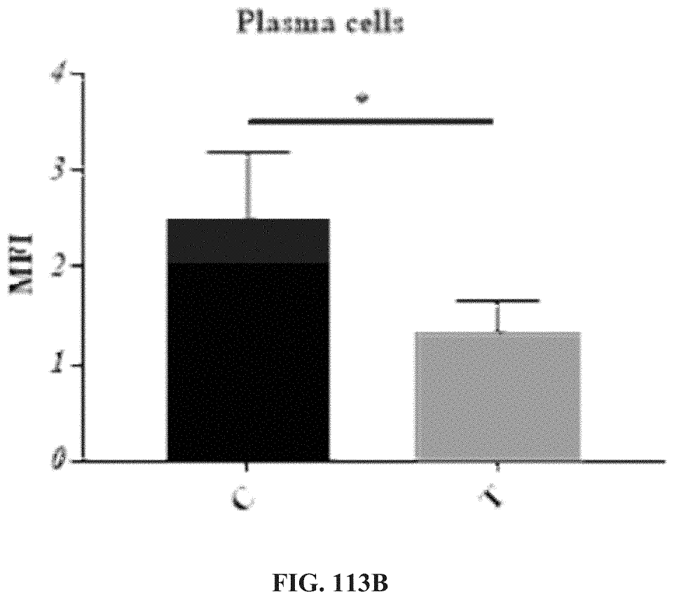

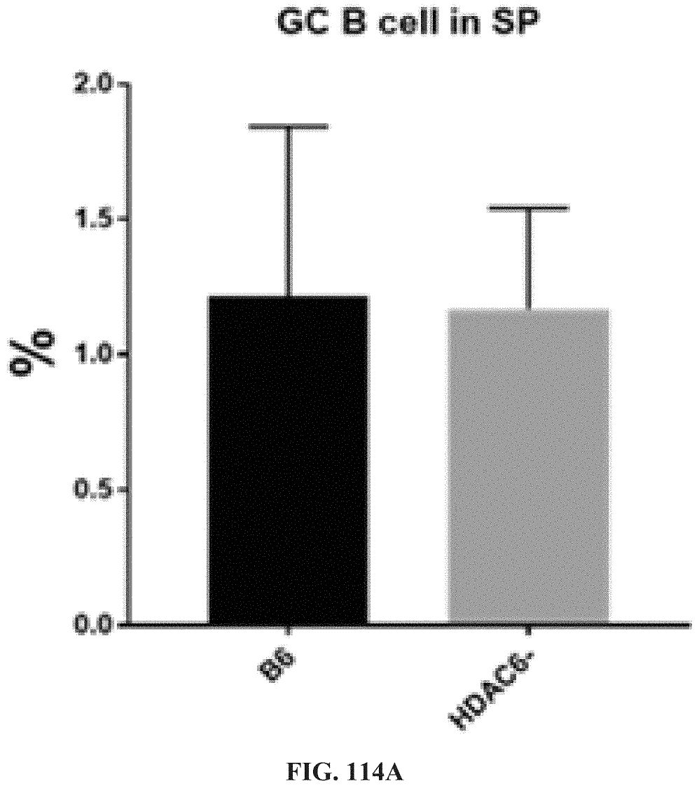

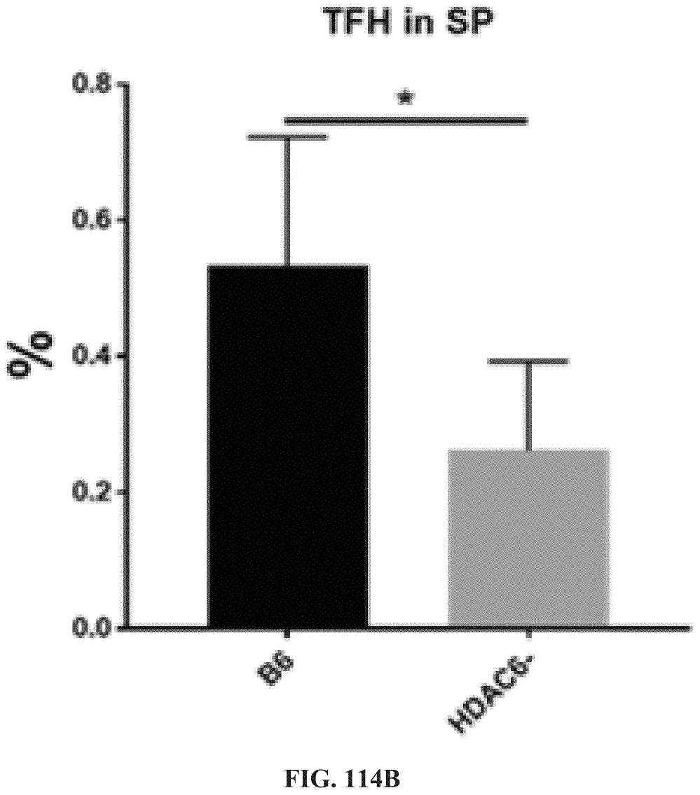

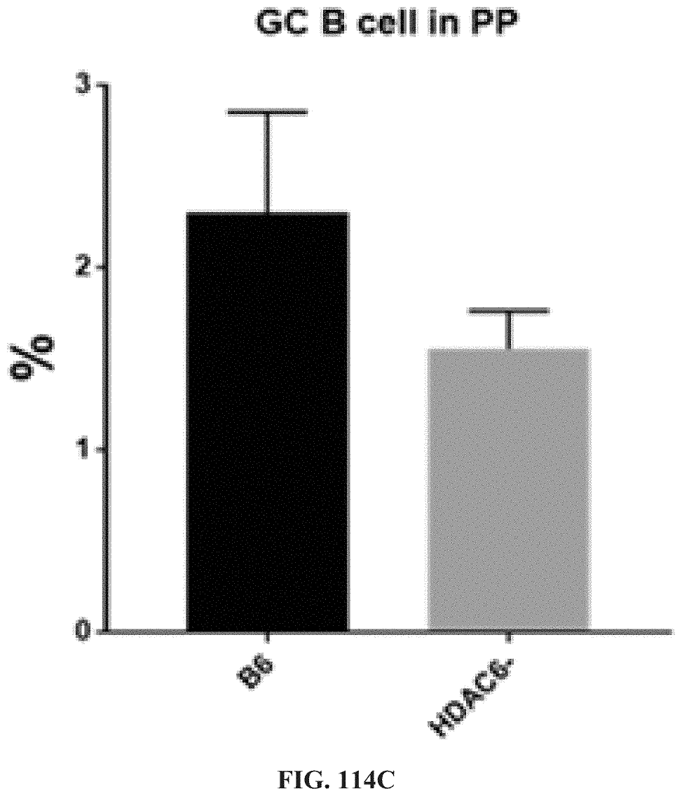

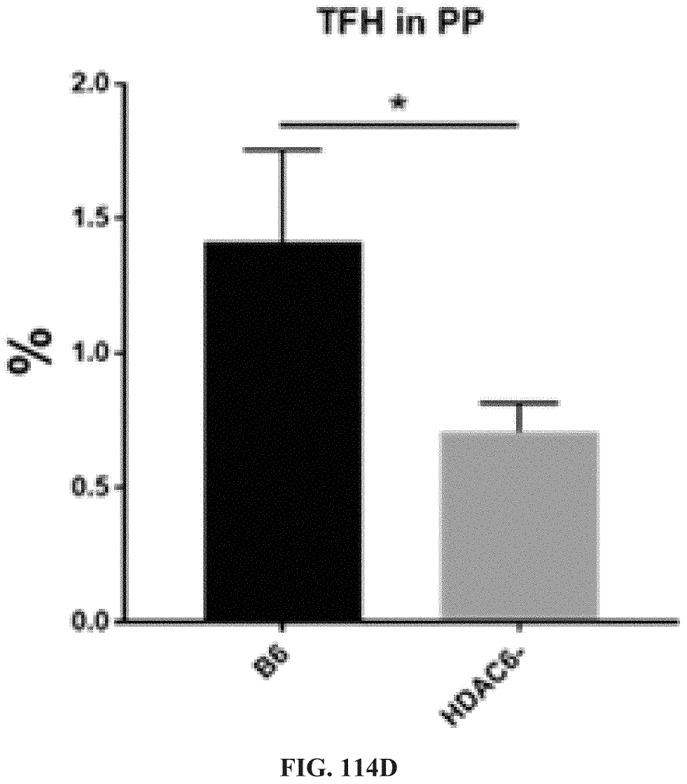

Next, gene set variation analysis (GSVA) was carried out to determine whether there was enrichment in transcripts identifying these populations. Indeed, it was observed that plasma cell, Tfh cell, and GC signatures were all decreased following 4 weeks of HDAC6 inhibitor treatment, as compared to the untreated control group (FIG. 107B). To validate further the impact of the HDAC6 inhibitor on germinal center B cell response, the changes of spleens and Peyer's patches from C57BL/6J^{HDAC6-/-} mice compared to C57BL/6J mice were assessed by flow cytometry (FIGS. 114A-114D). A reduction of T follicular helper cells (Tfh) in spleens and Peyer's patches of HDAC6 knockout mice was observed compared to wild-type C57BL/6J mice. Different from lupus-prone mice, the lack of HDAC6 in mice of B6 background showed no reduction of splenic spontaneous germinal centers in steady state. This shows that there are differences in molecular pathways in splenic germinal center formation in lupus mice compared to non-lupus prone mice. To confirm these RNA sequencing results, immunohistofluorescence (IHF) microscopy of splenic sections was performed to evaluate the presence of plasma cells and GCs (FIGS. 107C-107D). Consistent with the RNA sequencing results, both CD138⁺ PC (FIG. 107C) and PNA⁺ GC (FIGS. 107C-107D and 113A-113B) were dramatically reduced in the ACY-738-treated group, showing that HDAC6 treatment suppressed GC activity and subsequent PC generation and/or survival.

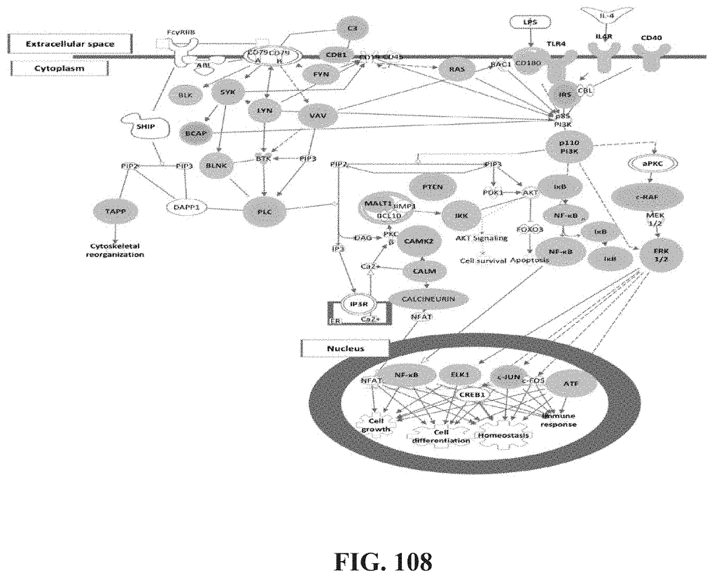

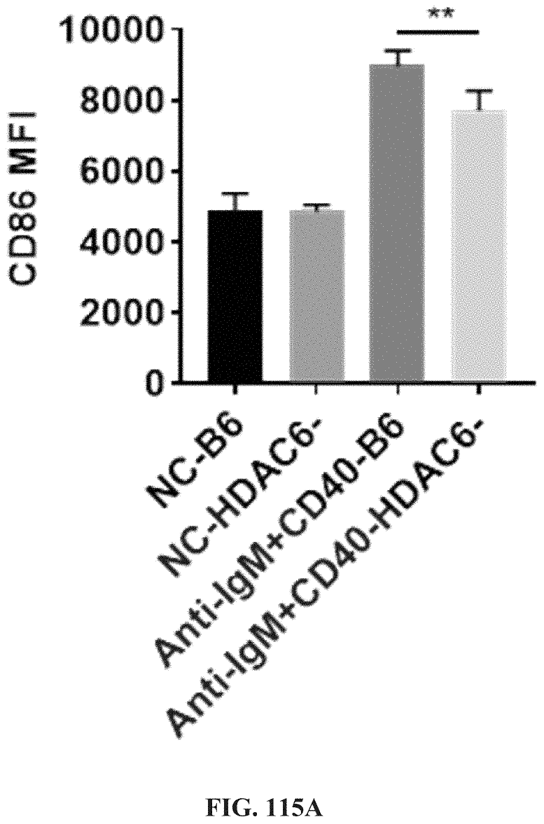

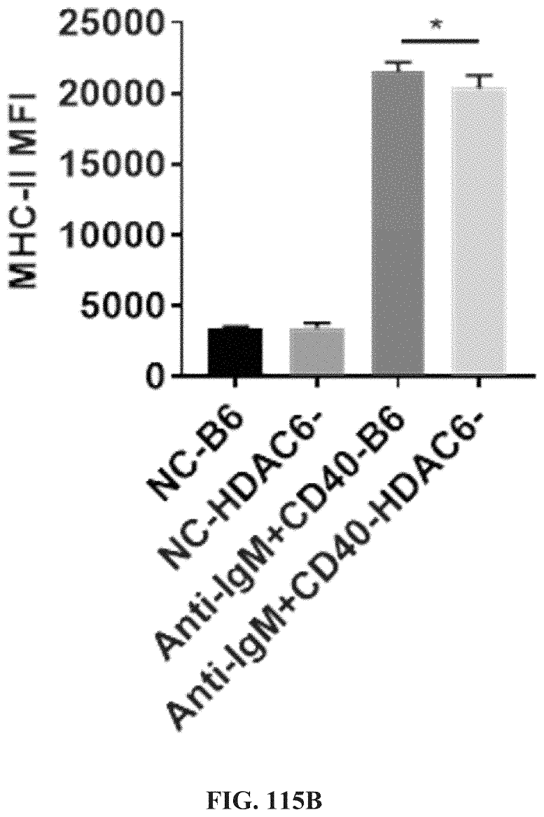

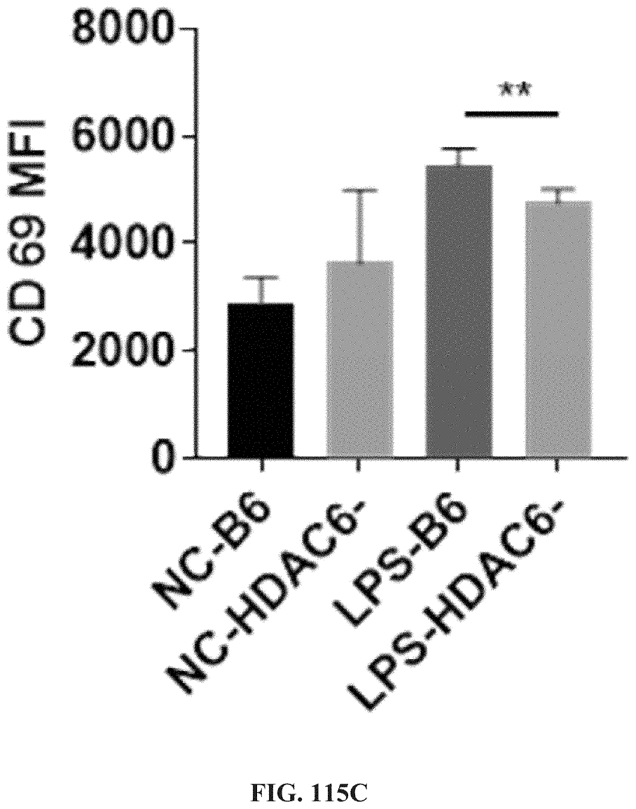

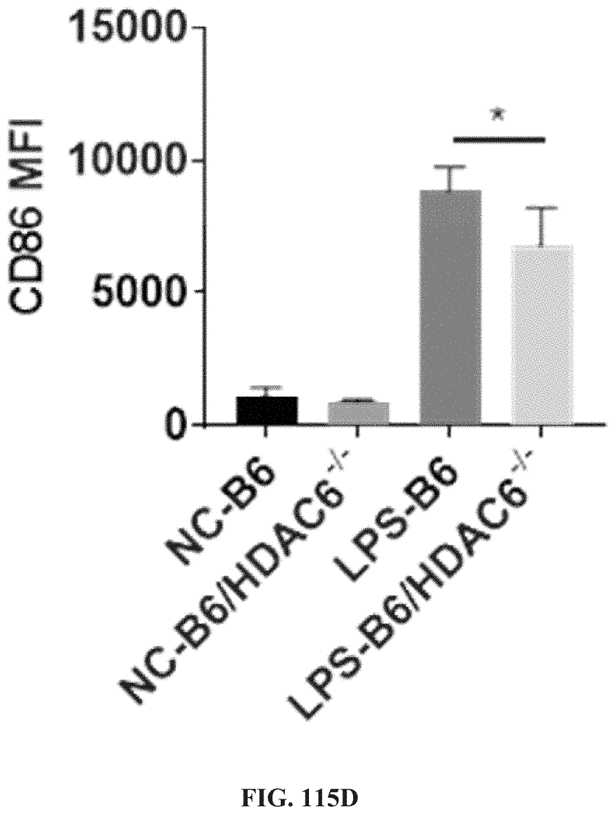

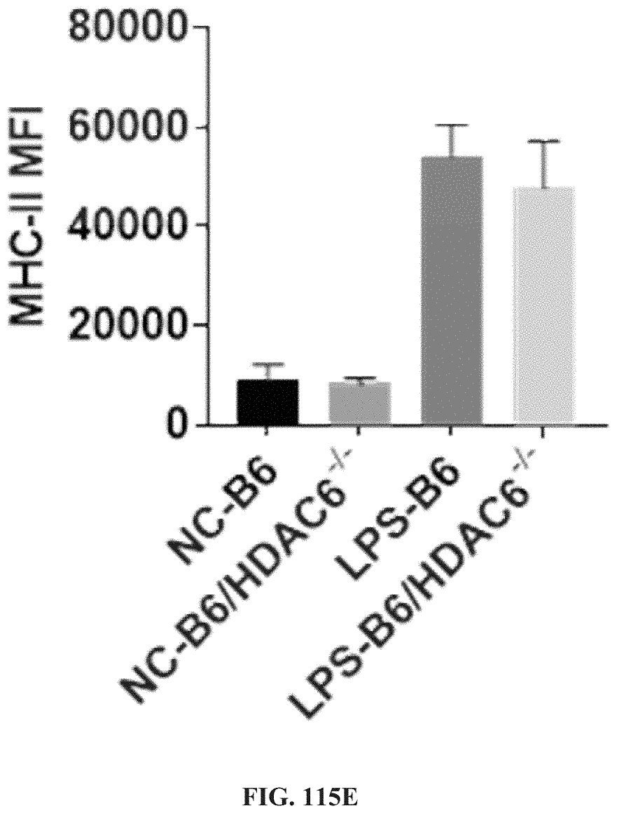

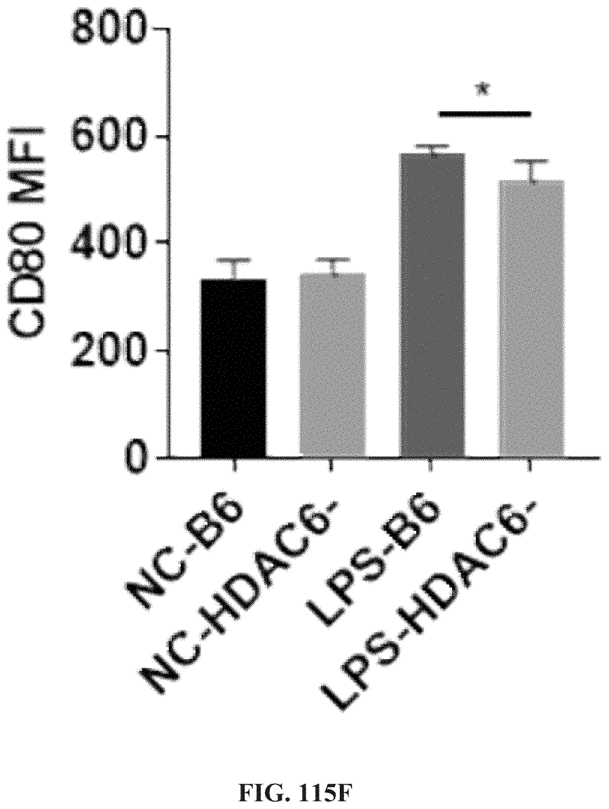

Further, the results showed that HDAC6 inhibition reduces B cell signaling in NZB/NZW F1 mice. To demonstrate that HDAC6 inhibition specifically inhibits B cell signaling, IPA canonical pathway analysis was employed to assess the pattern of change in differential gene expression in HDAC6-treated mice (FIG. 108). HDAC6 inhibition was found to reduce transcripts involved in both the BCR and the TLR dependent PI3K signaling pathway in B cells, as well as decreasing transcription factors, NF-κB, ELK1, c-JUN, and ATF, which control cell growth, differentiation, and homeostasis of many types of cells, including B cells. To validate the role of HDAC6 in regulation of B cell activation signaling showed by analysis of RNAseq data, in vitro stimulation experiments were performed with HDAC6^{-/-} mice and NZB/W mice (FIGS. 115A-115F and 116A-116F). Reduced activation of B cells was observed in B cells from C57BL/6J/HDAC6^{-/-} mice as well as ACY-738 treated NZB/W mice.

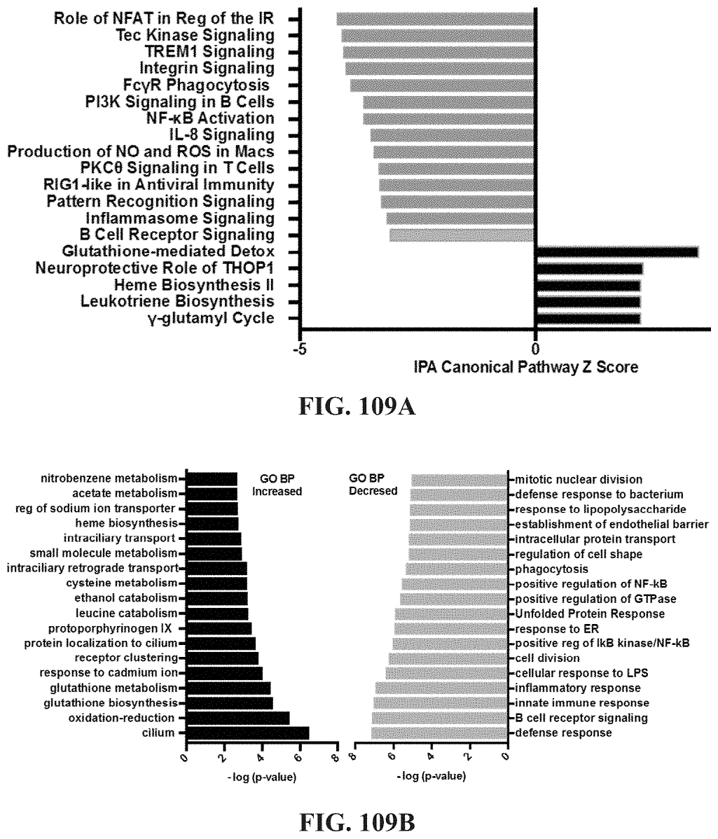

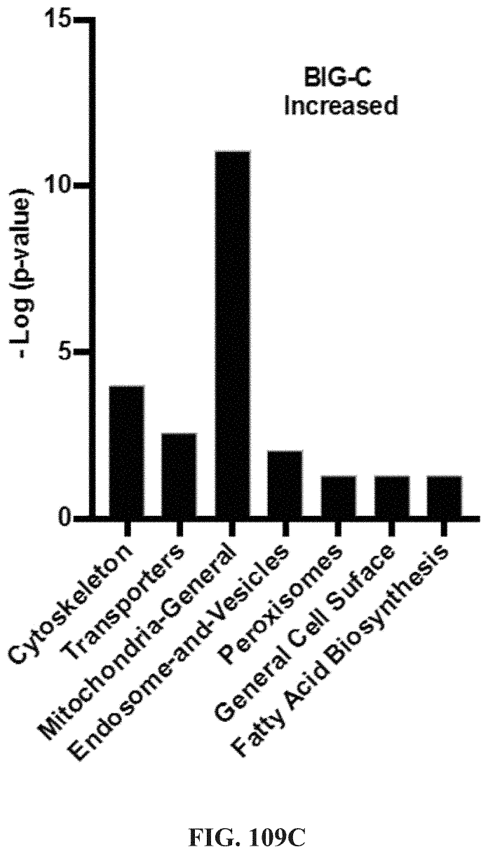

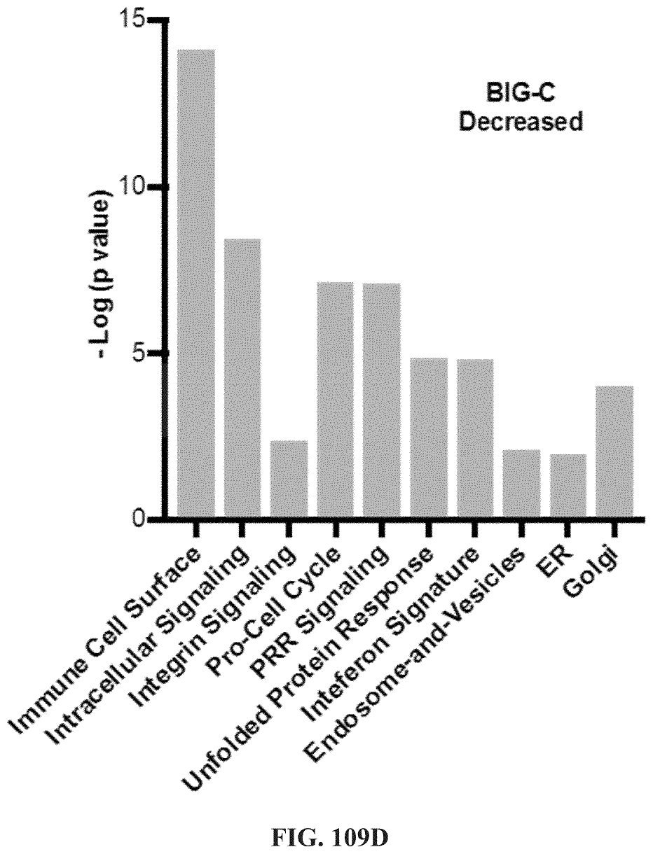

Further, the results showed that HDAC6 inhibition alters gene transcripts associated with inflammation and cellular metabolism. To investigate further the specific pathways by which HDAC6 inhibition decreased the molecular basis of lupus, several additional analyses were carried out (FIGS. 109A-109D). IPA was used to determine the biological pathways significantly affected by HDAC6 inhibitor treatment (FIG. 109A). There were only five significant signaling pathways increased by HDAC6 inhibitor treatment (p≤0.05; Z≥2). It was observed that HDAC6 inhibition led to an increase in glutathione metabolism and the gamma-glutamyl cycle, which may be related to the activation of the mercapturic acid pathway for the detoxification of foreign compounds. With regard to pathways down-regulated with HDAC6 inhibition, there were 59 pathways with Z scores≤−2 and p values≤0.05; pathways associated with immune signaling, B cell signaling, myeloid inflammatory pathways and phagocytosis—all pathways that may be important in the pathogenesis of human SLE.

Next, GO biological pathway enrichment analysis was carried out separately on increased and decreased transcripts and categories with significant overlap p values were determined (FIG. 109B). GO biological pathway analysis confirmed the increase in biochemical processes associated with drug metabolism shown by IPA, but processes related to cilium assembly were most highly enriched. The most decreased GO categories were related to the immune and inflammatory response, B cell receptor signaling, cell division, ER stress and unfolded protein responses, NF-κB signaling and phagocytosis. Furthermore, a decrease in the interferon gene signature as well as pattern recognition receptors such as TLRs was also observed. These results illustrate that the IPA pathways and the GO biological pathway analysis showed similar changes in transcription and signaling profiles.

Next, the enrichment of transcripts increased or decreased in HDAC6-treated NZB/NZW mice were assessed using the BIG-C clustering algorithm and chi square analysis to evaluate significant enrichment of BIG-C categories (FIG. 109C). In agreement with results of human patients treated with HDAC inhibitors, a significant metabolic shift was observed, as evidenced by the increase in biochemical markers in the cytoplasm, including enzymes associated with fatty acid synthesis, mitochondrial and peroxisome activity (Table 65). Furthermore, the observed decrease in transcripts associated with the unfolded protein response, golgi, ER, and cell cycle transcripts associated with recently generated plasma cells support an overall reduction in plasma cells (Table 66). Thus, the three analytical methodologies demonstrated that HDAC6 inhibitor treatment led to increased transcripts associated with biochemical pathways and cytoskeletal events, and decreased transcripts associated with plasma cells and immune networks.

Further, the results showed that HDAC6 inhibition alters cellular metabolism. For immune cells to become activated, metabolic processes increase to support activation, proliferation, and differentiation. Although pathways associated with the mitochondria and cellular biochemistry were affected by HDAC6 inhibition, it was unclear whether a specific type of metabolism was predominating after treatment. Increased transcripts related to cellular energy production demonstrated nine genes associated with glycolysis (Fbp1 (negative regulator), Ier3 (negative regulator), G6pc3, Pfkm, Aldoc, Dhktd1, Prkaa2, Khk, Eno2), 12 genes involved in oxidative phosphorylation (Taz, Atp5s, Slc25a23, Cox4l2, Cox6b2, Ndufb3, mt-Nd2, mt-Nd4, mt-Cytb, Nipsap2, Coq7 and Nubp1), seven fatty acid beta-oxidation genes (Acsbg1, Slc27a6, Slc27a1, Ivd, Pex5, Pex7, Hadh, Decr1, Echdc2, Acad11) and four genes associated with the TCA cycle (Pdk2 (negative regulator), Idh2, Sdhaf4, Dhtkd1). Among decreased transcripts, there were nine genes associated with glycolysis (Pgk1, Pgam1, Pfkfb3, Hk2, Pfkp (expressed in platelets and fibroblasts), Zbtb7a, Nupr1, Hif1a, Tpil1), seven with oxidative phosphorylation (Coa5, Nupr1, Pgk1, Atp7a, Bid, Vcp, Pde12), two with fatty acid beta oxidation (Abcd1, Abcd2), and four with the TCA cycle (Glud1, Idh1, Pdha1, Pdpr).

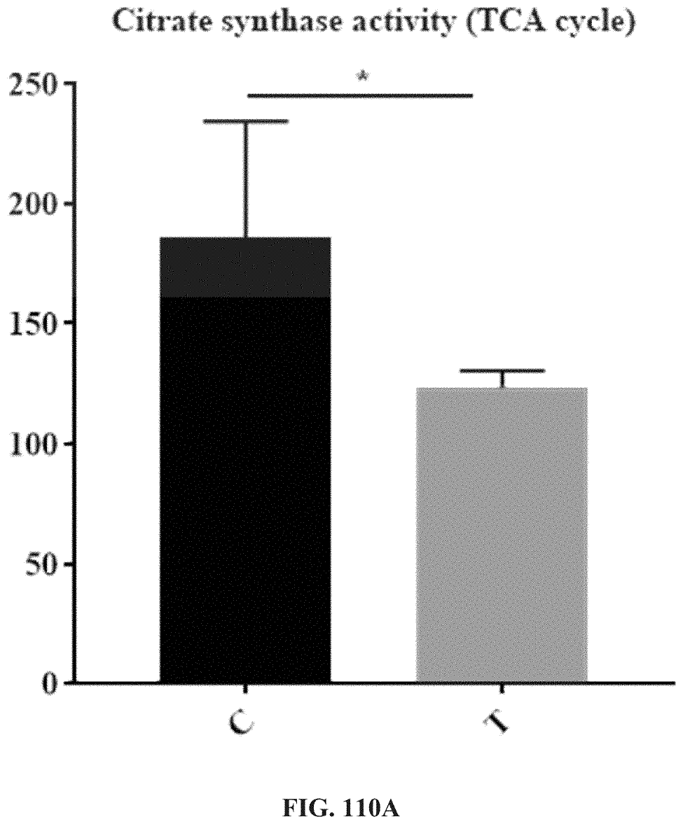

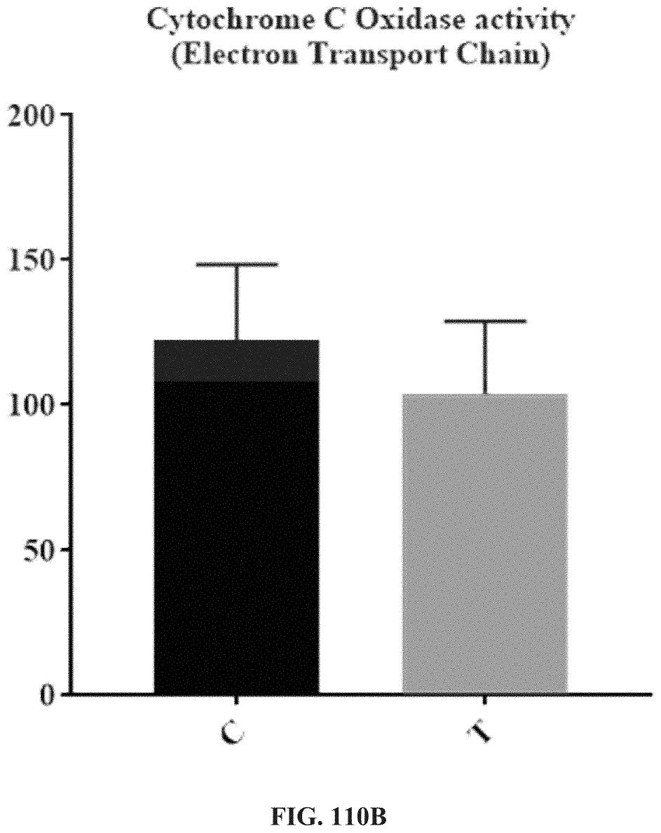

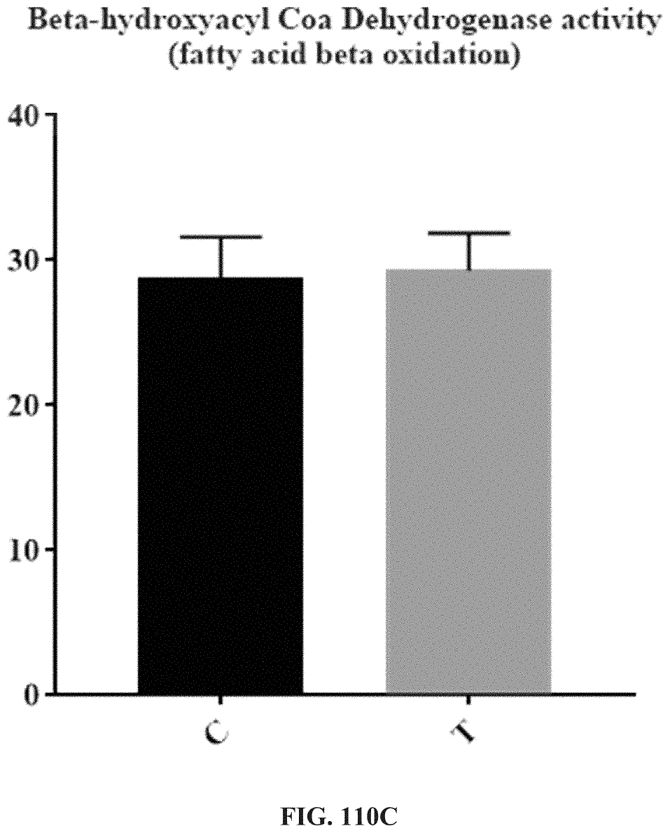

To determine whether the altered transcripts induced by HDAC inhibition led to altered metabolic pathways in lupus mice, the enzyme activity of proteins involved in electron transport chain function, the tricarboxylic acid cycle, and fatty acid beta oxidation was observed in the spleens of lupus mice treated with the HDAC6 inhibitor ACY-728 for a 4-week period (FIGS. 110A-110C). A significant decline was observed in citrate synthase enzyme function in response to HDAC inhibition (p=0.043). The activity of citrate synthase is a biochemical marker of mitochondrial density and oxidative capacity. The activities of beta hydroxyacyl CoA dehydrogenase (βHAD), a key regulatory enzyme in the beta oxidation of fatty acids to acetyl CoA, was unchanged with HDAC6 inhibition; further, cytochrome c oxidase, which is important in the function of mitochondrial electron transport chain function, was decreased, but not statistically significant (p=0.053).

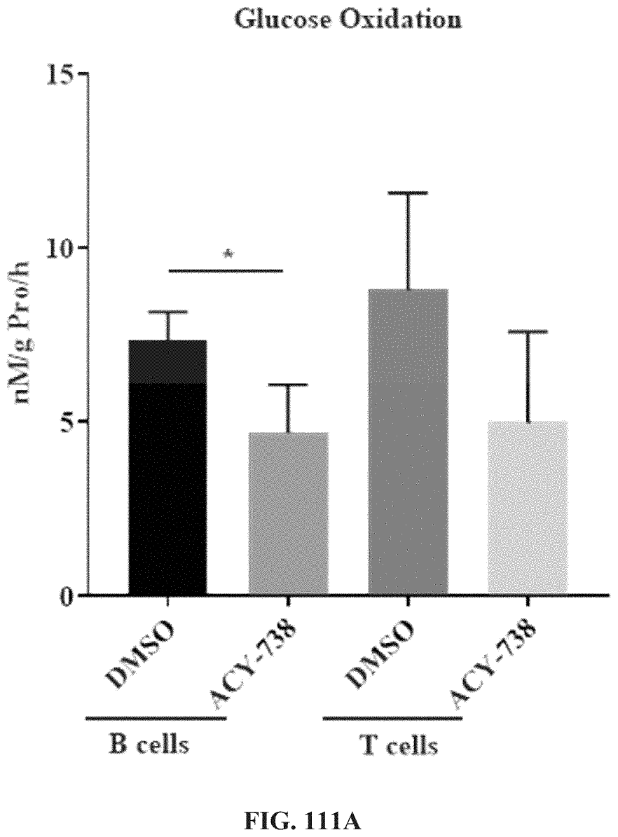

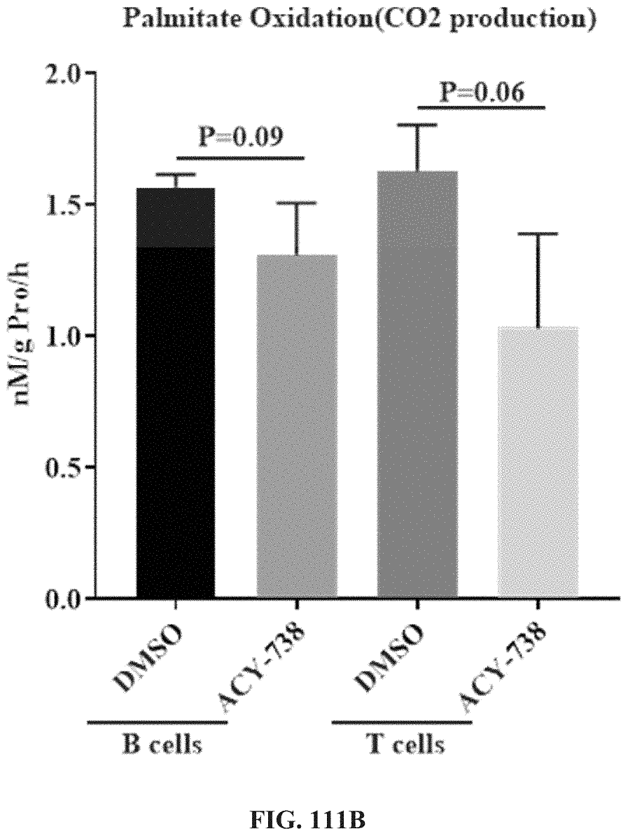

To investigate further the role of ACY-738 on the metabolic function of B and T cells, in vitro experiments were performed on cells isolated from NZB/W female lupus mice. Purified B cells and T cells were stimulated with LPS or anti CD3/CD28 for 24 hours with or without 4 μM of ACY-738 (FIGS. 111A-111B); this concentration of ACY-738 is effective at inhibiting inflammatory mediator production and activation in immune cells without toxicity. Glucose is a major source for energy and biosynthesis in activated T and B cells. In the cell, glucose undergoes a 10-step reaction to generate pyruvate, which is either reduced into lactate by lactate dehydrogenase in the cytosol, or transported into the mitochondria via the mitochondrial pyruvate carrier complex, where it is converted into acetyl-CoA by the pyruvate dehydrogenase complex, a process that is tightly regulated by the pyruvate dehydrogenase kinase (Pdk1), which may phosphorylate pyruvate dehydrogenase complex and inhibit its activity. When B cells were treated with ACY-738, CO₂ produced from oxidation of glucose was significantly decreased (p=0.044). In T cells, there was a reduction in CO₂ after treatment, but it was not significant (p=0.16) (FIGS. 111A-111B) Next, the amount of CO₂ production from fatty acids (palmitate) was measured with and without ACY-738 treatment. Similarly, results showed that ACY-738 did not decrease CO₂ production from fatty acids in stimulated B cell and T cells significantly (P=0.09, B cells; and P=0.06, T cells).

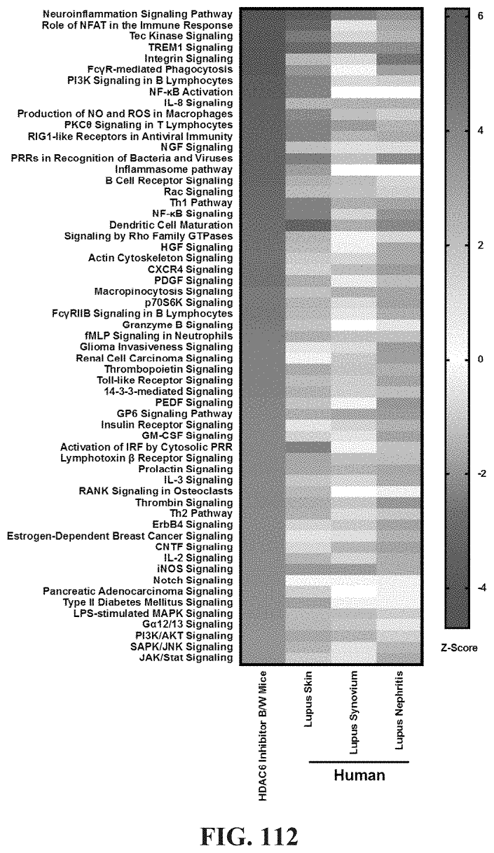

Further, the results showed that HDAC6 inhibition in mice decreases pathogenic signaling pathways that are up-regulated in active human SLE. In order to demonstrate the relevance of these findings regarding HDAC6 inhibitor-mediated suppression of molecular pathways in lupus mice, the down-regulated pathways were compared to those found to be up-regulated in active human lupus. Specifically, the pathways down-regulated by HDAC6 inhibition in NZB/W mice were compared to pathways up-regulated in human lupus affected organs, including skin, synovium, and kidney (FIG. 112). The results showed that the molecular pathways decreased by the HDAC6 inhibitor in NZB/W mice are also highly up-regulated in human SLE affected tissues. For example, ACY-738 treatment of NZB/W mice significantly decreased a total of 59 IPA canonical pathways (Z<−2, p<0.05). Of these pathways, 38 (64%) had significant positive Z scores (Z>2) for at least two of three human SLE affected tissues. For the remaining 21 IPA canonical pathways decreased by ACY-738 treatment, positive Z scores less than 2 were found for most of the human SLE affected tissues. ACY-738 treatment of mice increased a total of 5 canonical pathways, and none were significantly decreased in human SLE, although glutathione mediated detoxification (Z=3.5 in HDAC6 inhibitor-treated mice) had negative Z scores for human lupus skin (−1.8) and lupus nephritis (−1.6). The striking overlap in canonical pathways affected by HDAC6 inhibition and the aberrant pathways in human SLE affected tissues confirms the utility of the murine lupus results toward predicting potential benefit in human lupus.

Discussion

In the current studies, mechanisms by which HDAC6 inhibition decreases disease pathogenesis in NZB/W mice were investigated by using RNAseq to evaluate the transcriptomic signatures of splenocytes from treated mice and untreated control mice coupled with applied computational cellular and pathway analysis. In addition, the transcriptomic data obtained from the HDAC6 treated mice and human gene expression information were bridged to determine the relevance to this target in possibly controlling human lupus. Results showed that PC development was abrogated and GC formation was greatly reduced in HDAC6 inhibitor-treated NZB/W mice. When the HDAC6 inhibitor-treated lupus mouse gene signatures were compared to human lupus patient gene signatures, the results showed numerous immune and inflammatory pathways increased in active human lupus affected tissue were significantly decreased in the HDAC6 inhibitor-treated animals. Pathway analysis showed alterations in cellular metabolism may contribute to the normalization of lupus mouse spleen genomic signatures, and this was confirmed by direct measurement of the impact of the HDAC6 inhibitor on metabolic activities of murine spleen cells. Taken together, these studies show that HDAC6 may decrease germinal center activity and B cell activation, and reduces several signaling pathways required for PC differentiation in the context of LN. Moreover, the molecular pathways suppressed by the HDAC6 inhibitor were frequently overexpressed in human lupus tissue. Of importance, the results also show that HDAC6 inhibition corrects aberrant cellular metabolism observed in lupus.

There are numerous signaling pathways, metabolic events, and transcription factors that regulate the differentiation of B cells into PC. A rationale for continued investigations to define the molecular events in lupus immunopathogenesis mediated by HDAC6 is related to the uncertainty of the non-redundant roles of HDAC6 in immune function in general and lupus in particular. HDAC6 knock out mice (HDAC6^{-/-}) have grossly normal immune cell development. However, HDAC6^{-/-} mice show a four-fold decrease in antibody production in response to immunization with a T cell-dependent antigen. Furthermore, responses to RNA but not DNA viruses are reduced in HDAC6-deficient mice. HDAC6 is a unique member of the HDAC family that largely resides within the cytoplasm and regulates the acetylation status of a number of cytoplasmic proteins. These include proteins involved in the tubulin cytoskeleton as well as the proteasome. HDAC6 inhibition, therefore, has the potential to alter a variety of cellular functions. Inhibition of HDAC6 has also beneficial effects treating, for example, multiple myeloma, an expansion of malignant PCs that secrete abnormal antibodies. In lupus, HDAC6 may act to regulate both innate and adaptive immune responses. HDAC6 acts as a coactivator for interferon-beta (IFN-β) induction, and HDAC6 inhibition prevents IFN-β expression. Indeed, results showed that the IFN signature is decreased. β-catenin also serves a target of HDAC6; deacetylation of β-catenin facilitates it translocation to the nucleus to serve as a co-activator for IRF3-mediated transcription, a possible mechanism for its impact on IFN-β production. In B cells, HDAC6 inhibition leads to the acetylation of NFκB which prevents its nuclear translocation. Alpha tubulin regulates the cellular cytoskeleton and is acetylated by HDAC6 inhibitors. Increased acetylation of alpha tubulin may inhibit the B-T cell interaction by preventing B cell migration and germinal center formation. Indeed, Tfh-B cell collaboration requires interaction of CD40L and IL-4 with CD40 and IL-4L, respectively. Further, HDAC6 inhibition may be shown to result in a decreased Tfh population and reduced CD40 and IL-4L activities in B cells. The results described herein confirm that HDAC6 inhibition decreased the Tfh population in lupus mice. Additionally, regulation of B cell activation involves tyrosine kinase regulation. P85/P110-PI3K belongs to class IA PI3K mediated signals which regulate B cell commitment and differentiation. PI3K signaling pathways may be activated in a Toll like receptor (TLC)-dependent or B cell receptor (BCR)-dependent manner. Following treatment with ACY-738, results showed decreased PI3K transcripts which are important for B cell inflammatory signaling. Bruton's tyrosine kinase (Btk) is also an important component of BCR signaling. Of note, increased Btk expression may be observed in human autoimmune disease. Bkt activation may control the entry of peripheral naïve B cells into the follicle, survival and maturation of B cells, and plasma cell differentiation. Further, inhibition of Btk may reduce autoantibody production and pathogenesis. Btk inhibition may reduce B cell activation, differentiation of PC and autoantibody class-switching. The current results showed that Btk expression and signaling cascade was suppressed by HDAC6 inhibition, and the suppression of Btk may have occurred through inhibition of PI3K signaling. In summary, the HDAC6 inhibitor suppresses expression of a number of pathways that are essential for B cell activation and differentiation of PC. The therapeutic effect in SLE may based on inhibition of one or more activation pathways required for germinal center formation and PC differentiation and survival.

HDAC6 inhibitor treatment was also demonstrated to have an effect on cellular metabolism. This was shown in vivo in treated mice and in vitro with cultured lymphocytes. In regard to cellular metabolism, results showed a significant metabolic shift as evidenced by the increase in gene expression profiles of biochemical markers in the cytoplasm, including mitochondrial enzymes associated with fatty acid oxidation and peroxisome activity which may be reported with HDAC6i. Despite an increase in mRNA content of mitochondrial enzymes, results showed a significant decline in citrate synthase enzyme function in response to HDAC6 inhibition. The activity of citrate synthase is a biochemical marker of mitochondrial density and oxidative capacity. Perhaps the increased gene expression signature is compensatory to a reduced enzyme activity. Indeed, it may be shown that mitochondrial metabolism, including citrate synthase activity, is down-regulated in response to HDAC6 inhibition. This is an important finding as 02 consumption may be found to be increased in SLE patents relative to control subjects. Furthermore, the electron transport chain complex I may be identified as a main source of oxidative stress in SL. B cell differentiation to PC requires a terminal increase in oxidative phosphorylation in order to generate antibodies. The activities of beta hydroxyacyl coA dehydrogenase (βHAD), a key regulatory enzyme in the beta oxidation of fatty acids to acetyl CoA, was unchanged with HDAC6 inhibition, whereas cytochrome c oxidase, important in the function of the mitochondrial electron transport chain function, was decreased but not significantly. Metabolic control of mitochondrial ROS production and glucose utilization may be recognized as regulators of cellular activation within T cells. In particular, glucose utilization via the pentose phosphate pathway (PPP) and output of NADPH may regulate the mitochondrial transmembrane potential during T cell activation, and chronic activation of CD4⁺ T cells from lupus-prone mice and SLE patients may occur with high levels of oxygen consumption. Indeed, in other immune-mediated inflammatory diseases, an increased activation of the citric acid cycle may be associated with disease. Taken together, these results show that HDAC6 inhibition may decrease lupus disease by regulating immunologic as well as metabolic function.

To investigate further whether HDAC6 inhibition directly decreased cellular metabolism or whether the changes noted in treated animals were secondary to quieting of the immune response, NZB/W B and T cells were stimulated in vitro and with and without the HDAC6 inhibitor ACY-738. Results showed that glucose metabolism was significantly decreased in B cells and that fatty acid oxidation was also reduced with HDAC6 inhibition. Combining these gene expression results along with the in vitro metabolic results show that glucose metabolism is critical for immune cell activation and inflammatory cytokine production. Human CD4⁺ T cells may show up-regulation in metabolism, including pyruvate oxidation and TCA cycle utilization, resulting in cell polarization and production of IFN-γ production. The in vitro results show that ACY-738 may limit cell metabolism and decrease the spontaneous activation of lupus T and B cells.

In summary, results show that selective HDAC6 inhibition corrects abnormal B cell activation and differentiation in NZB/W mice that display early onset disease. The correction in B cell differentiation and activation correlated with less severe renal disease. Specifically, HDAC6 inhibition decreased several signaling pathways that are critical for B cells differentiation to PC. In addition to HDAC6 inhibiting B cell and T cell activation, several metabolic and enzymes pathways that are observed to be increased in active lupus were also ameliorated. This was demonstrated through results and data obtained via both in vivo experiments and in vitro experiments. Finally, when RNA profiles from the NZB/W mice were compared to humans with lupus, the results demonstrate that the many of genes up-regulated in human lupus patients were decreased in lupus mice treated with HDAC6 inhibition. Taken together, these studies show that selective HDAC6 inhibition may be a potential therapeutic for the treatment of human patients with lupus nephritis (LN).

FIGS. 105A-105E show a non-limiting example of results showing that inhibition of histone deacetylase HDAC6 reduced Ig and C deposition in NZB/W lupus nephritis. FIGS. 105A-105B show a representative Hematoxylin and Eosin (H&E) staining image of kidney glomerular region along with pathology score which reflects the severity of membranoproliferative changes and distribution. FIG. 105C shows a representative immunohistological staining of kidney section for IgG and C3. FIGS. 105D-105E show a graphic analysis of mean fluorescent intensity (MFI) of IgG and C3. Data are shown as mean±standard error of the mean (s.e.m) n=4 mice for each group; T-test; *P<0.05, **P<0.01, ****P<0.0001.

FIG. 106 shows a non-limiting example of results showing that HDAC6i treatment of NZB/NZW F1 mice induced global gene expression changes in whole splenocytes. Hierarchical clustering of 3911 transcripts (1922up, 1989 down) that differed significantly (FDR<0.1) between control (C1, C3, C4, and C5) and treated mice (T1, T2, T3, and T5).

FIGS. 107A-107D show a non-limiting example of results showing that HDAC6i treatment results in significantly decreased GC activity and PC formation. FIG. 107A shows results of I-Scope hematopoietic cell enrichment demonstrating that HDAC6 inhibition decreased PC, B cells, and inflammatory myeloid cells. The numbers of transcripts corresponding to each cell type increased or decreased after HDAC6 inhibitor treatment are shown. Gene symbols for transcripts for PC, B cells, and inflammatory myeloid cells are shown in Table 59 (increased transcripts) and Table 60 (decreased transcripts). FIG. 107B shows results of GSVA analysis performed to determine the enrichment of PC, Tfh cells, and GC in each HDAC6 inhibitor-treated and control NZB/NZW mouse (Methods lists genes used for GSVA enrichment modules). FIG. 107C shows a representative splenic section stained with anti-CD138, anti-IgM, and PNA. FIG. 107D shows a representative splenic section stained for T cells, follicular B cells, and GC with anti-CD3, anti-IgD, and PNA.

TABLE 59
Gene Symbols from I-Scope Categories Increased
by HDAC6 Inhibitor Treatment

| | | Monocytes/ | | Plasma |
|---|---|---|---|---|
| B cells | Dendritic | Macrophages | Myeloid | Cells |
| 6 | 4 | 7 | 1 | 0 |
| Cd1d1 | Il15 | Ier3 | Clec4b2 | |
| Gng7 | Cd1d1 | Ccl17 | | |
| Vpreb1 | Cd209a | Mfge8 | | |
| Ly6d | Osm | Clec9a | | |
| Fam129c | | Il15 | | |
| Cd37 | | Mgl2 | | |
| | | Lgals9 | | |

TABLE 60
Gene Symbols from I-Scope Categories Decreased by HDAC6 Inhibitor Treatment

| | Monocytes/ | | | | | |
|---|---|---|---|---|---|---|
| | Macro- | | | Plasma | More | More |
| B cells | phages | Dendritic | Myeloid | Cells | plasma | Plasma |
| 25 | 44 | 16 | 15 | 148 | cells | Cells |
| Tnfrsf8 | Tlr8 | Cd300e | Ms4a4a | Jchain | Ighv1-54 | Igkv10-94 |
| Slamf1 | Tnfaip3 | Vsig4 | Clec7a | Hmmr | Ighv1-56 | Igkv10-95 |
| Havcr1 | Tnip3 | Hmmr | Pik3ap1 | Hvcn1 | Ighv1-58 | Igkv12-41 |
| Tlr7 | Ace | Igsf6 | Btk | Cd38 | Ighv1-61 | Igkv12-46 |
| Irf4 | Ms4a4a | Adamdec1 | Fgr | Slamf7 | Ighv1-62-3 | Igkv13-85 |
| Tlr9 | Clec4e | Il18bp | Bach1 | Hyou1 | Ighv1-63 | Igkv15-103 |
| Rgs13 | Cd300e | Il12b | Treml4 | Fkbp11 | Ighv1-66 | Igkv17-121 |
| Aicda | Clec4a3 | Il27 | Ms4a6c | Mzb1 | Ighv1-7 | Igkv17-127 |
| Sh2b2 | Tnfrsf1b | Themis2 | Itgax | Ighg1 | Ighv1-73 | Igkv2-109 |
| Samsn1 | Vsig4 | Cd180 | Apoc1 | Igkc | Ighv1-75 | Igkv2-116 |
| Pou2af1 | Lilra5 | Slamf1 | Slpi | Tnfrsf17 | Ighv1-76 | Igkv2-137 |
| Btk | Ms4a2 | Il21r | Gm15931 | Ighd | Ighv1-77 | Igkv3-1 |
| Klhl6 | Tgm2 | Cd83 | Lilrb4a | Stil | Ighv1-78 | Igkv3-11 |
| Pkn1 | Msr1 | Cnr2 | Cd300lb | Parpbp | Ighv1-83 | Igkv3-12 |
| Blk | Hmmr | Ulbp1 | Cd300ld | Ighd1-1 | Ighv1-84 | Igkv3-2 |
| Blnk | Pilra | Fcgr1 | | Ighd2-7 | Ighv10-1 | Igkv3-4 |
| Gcsam | Igsf6 | | | Ighe | Ighv13-2 | Igkv3-9 |
| Tnfsf8 | Siglec1 | | | Ighg3 | Ighv14-2 | Igkv4-51 |
| Tnfrsf13c | Clec4d | | | Ighj1 | Ighv14-3 | Igkv4-57-1 |
| Cd22 | Fpr2 | | | Ighj2 | Ighv14-4 | Igkv4-58 |
| Nuggc | Csf1r | | | Ighj3 | Ighv2-2 | Igkv4-61 |
| Elf1 | Clec7a | | | Ighv1-12 | Ighv2-3 | Igkv4-63 |
| Snap23 | Adgre1 | | | Ighv1-14 | Ighv2-4 | Igkv4-69 |
| Ncf1 | Smpdl3b | | | Ighv1-18 | Ighv2-6 | Igkv4-70 |
| H2-Ob | Adamdec1 | | | Ighv1-19 | Ighv2-6-8 | Igkv4-73 |
| | Cd5l | | | Ighv1-2 | Ighv2-7 | Igkv4-74 |
| | Cfb | | | Ighv1-20 | Ighv2-9 | Igkv4-77 |
| | Il18bp | | | Ighv1-21 | Ighv2-9-1 | Igkv4-86 |
| | Il10 | | | Ighv1-21-1 | Ighv3-3 | Igkv5-37 |
| | Il12b | | | Ighv1-22 | Ighv3-4 | Igkv5-39 |
| | Ctla2b | | | Ighv1-25 | Ighv3-6 | Igkv5-43 |
| | Il27 | | | Ighv1-26 | Ighv5-12 | Igkv5-45 |
| | C6 | | | Ighv1-28 | Ighv5-15 | Igkv6-14 |
| | Serping1 | | | Ighv1-30 | Ighv5-16 | Igkv6-23 |
| | Cybb | | | Ighv1-31 | Ighv5-17 | Igkv6-29 |
| | Cxcl10 | | | Ighv1-33 | Ighv5-2 | Igkv6-32 |
| | Slc11a1 | | | Ighv1-36 | Ighv5-4 | Igkv8-21 |
| | Lmnb1 | | | Ighv1-37 | Ighv5-6 | Igkv8-24 |
| | Hvcn1 | | | Ighv1-39 | Ighv5-9 | Igkv8-28 |
| | Mpeg1 | | | Ighv1-4 | Ighv5-9-1 | Igkv8-30 |
| | Clec4n | | | Ighv1-42 | Ighv6-5 | Igkv9-120 |
| | Csf2rb2 | | | Ighv1-43 | Ighv6-6 | Igkv9-123 |
| | Lyz1 | | | Ighv1-47 | Ighv7-1 | Igkv9-124 |
| | Fcgr1 | | | Ighv1-5 | Ighv7-2 | Igkv9-129 |
| | Fcgr3 | | | Ighv8-8 | Ighv7-3 | Iglc4 |
| | | | | Igip | Ighv7-4 | Igll1 |
| | | | | Igkj2 | Ighv8-11 | Igkj5 |
| | | | | Igkj3 | Ighv8-2 | Igkv1-117 |
| | | | | | Ighv8-4 | Igkv1-122 |
| | | | | | Ighv8-5 | Igkv1-99 |

FIG. 108 shows a non-limiting example of results showing that HDAC6 inhibition repressed B cell signaling pathways in NZB/NZW mice. The IPA Canonical Signaling Pathway “B Cell Receptor Signaling” had a Z score of −3.1. Transcripts differentially expressed between HDAC6 inhibitor-treated and untreated NZB/NZW mice were overlaid on genes in the IPA pathway. Decreased transcripts are shown in green, while increased transcripts are shown in pink.

FIGS. 109A-109D show a non-limiting example of results showing that inhibition of HDAC6 altered transcripts associated with cellular metabolism. FIG. 109A shows results of an ingenuity pathway analysis (IPA) performed on the differentially expressed transcripts between HDAC6 inhibitor-treated and untreated NZB/NZW mice. The most significant signaling pathways increased or decreased by Z score analysis with an overlap p value≤0.05 are shown. The full list of significant increased and decreased pathways and the genes used to determine significance are in Table 61 (increased) and Table 62 (decreased). FIG. 109B shows results of a GO biological pathway enrichment analysis of the top most increased and decreased pathways by lowest overlap p value significance. A full list of GO biological pathways enriched (p<0.01) are in Table 63 (increased) and Table 64 (decreased). FIGS. 109C-109D show results of a BIG-C pathway enrichment performed using increased (FIG. 109C) or decreased (FIG. 109D) transcripts from the DE analysis of HDAC6 inhibitor-treated NZB/NZW mice compared to NZB/NZW mice. The −log (p value) is shown for the enriched categories. Gene symbols corresponding to each category are listed in Table 65 (increased) and Table 66 (decreased).

TABLE 61
Ingenuity (IPA) Canonical Pathways with Positive Z Scores

| | -log(p- | | z- | |
|---|---|---|---|---|
| Pathway | value) | Ratio | score | Molecules/Genes |
| Glutathione- | 2.97 | 0.387 | 3.464 | GSTZ1, GSTM2, MGST2, GSTM5, GSTM3, GGH, |
| mediated | | | | GSTM4, Gsta4, GSTO2, MGST3, GSTP1, GSTK1 |
| Detoxification | | | | |
| Neuroprotective | 1.56 | 0.218 | 2.294 | PRSS50, HLA-A, PRSS12, CTSG, PRSS33, |
| Role of THOP1 | | | | SERPINA3, PRSS41, PRKAG1, C1R, PRSS16, |
| in Alzheimer's | | | | PRSS36, LONP1, Prss30, ACE, GZMA, GZMK, |
| Disease | | | | ENDOU, KLK8, TPSAB1/TPSB2, HPN, APP, PREP, |
| | | | | PRKAR2B, PRTN3, MART, ST14 |
| Heme | 2.26 | 0.556 | 2.236 | UROD, PPOX, UROS, ALAD, HMBS |
| Biosynthesis II | | | | |
| Leukotriene | 1.47 | 0.385 | 2.236 | MGST2, GGT5, GGT1, MGST3, GGT7 |
| Biosynthesis | | | | |
| γ-glutamyl | 3.5 | 0.571 | 2.121 | CHAC1, GGT5, GCLM, GGT1, GSS, CHAC2, |
| Cycle | | | | GGACT, GGT7 |

TABLE 62
Ingenuity (IPA) Canonical Pathways with Negative Z Scores

| | -log(p- | | z- | |
|---|---|---|---|---|
| Pathway | value) | Ratio | score | Molecules/Genes |
| Neuroinflammation | 3.88 | 0.228 | −4.695 | TRAF3, TGFBR1, AGER, TICAM2, TGFBR3, |
| Signaling Pathway | | | | TLR8, NCSTN, MAPK13, CX3CR1, CXCL10, |
| | | | | TGFBR2, IKBKB, IKBKG, PIK3CG, MAPK3, |
| | | | | CYBB, PLA2G4F, PLA2G12A, FGFR1, NFKB2, |
| | | | | MAPK12, TLR9, IRF7, PTPN11, GAB1, CD40, |
| | | | | PLCG2, MAPT, SYK, GAD1, SLC6A1, PIK3R6, |
| | | | | CFLAR, CX3CL1, ICAM1, HLA-A, PIK3R5, |
| | | | | MFGE8, FZD1, NFKB1, SLC6A13, GRINA, |
| | | | | PLA2G4E, JUN, TLR7, CASP8, NOS2, PPP3CA, |
| | | | | NLRP3, PIK3C2A, GRB2, IL10, MYD88, MAPK6, |
| | | | | ACVR1, IL1R1, IRAK3, CSF1R, XIAP, APP, |
| | | | | PLA2G4A, TLR4, RIPK1, IL12B, H2-Eb2, HLA- |
| | | | | DOB, Tlr13, TIRAP, IRAK4, PSEN1, BIRC2 |
| Role of NFAT in | 3.52 | 0.247 | −4.217 | BLNK, RAF1, FYN, Calm1 (includes others), |
| Regulation of the | | | | HLA-A, NFKBIE, GNB5, PIK3R5, CSNK1A1, |
| Immune Response | | | | FCER1A, KRAS, GNA14, NFKB1, FCGR1A, |
| | | | | GNG7, IKBKB, IKBKG, GNG11, JUN, PIK3CG, |
| | | | | MAPK3, XPO1, GNA13, FCGR3A/FCGR3B, |
| | | | | PPP3CA, CALML5, PIK3C2A, FCGR2A, GRB2, |
| | | | | FGFR1, CSNK1G3, NFKB2, GNAZ, TLR9, BTK, |
| | | | | GAB1, PTPN11, H2-Eb2, PLCG2, SYK, LYN, |
| | | | | PIK3R6, MS4A2, HLA-DOB, MEF2C, GNAL |
| Tec Kinase | 2.98 | 0.241 | −4.116 | FYN, GNB5, PIK3R5, FCER1A, GNA14, NFKB1, |
| Signaling | | | | GNG7, BLK, PAK1, ITGA3, GNG11, RHOB, |
| | | | | PIK3CG, HCK, GNA13, ACTG2, FGR, PRKCA, |
| | | | | VAV2, STAT6, PIK3C2A, GRB2, FGFR1, NFKB2, |
| | | | | STAT3, GNAZ, MAPK12, TLR9, BTK, TLR4, |
| | | | | RHOQ, GAB1, PTPN11, PRKCD, PLCG2, LYN, |
| | | | | PIK3R6, MS4A2, VAV1, STAT2, GNAL |
| TREM1 Signaling | 3.82 | 0.32 | −4.082 | ICAM1, NLRP3, IL10, GRB2, MYD88, CIITA, |
| | | | | LAT2, TLR8, CD83, STAT3, NFKB2, NFKB1, |
| | | | | TLR9, NLRC5, TLR4, NOD2, MPO, CD40, |
| | | | | MAPK3, NLRC3, PLCG2, TLR7, Tlr13, ITGAX |
| Integrin Signaling | 2.68 | 0.224 | −4.025 | RAF1, RAPGEF1, MAP3K11, KRAS, PTEN, |
| | | | | ITGB3, ITGAE, ITGA3, PAK1, RHOB, MAPK3, |
| | | | | PIK3CG, GRB7, ITGAV, ACTG2, CAPN5, ACTR2, |
| | | | | CRKL, FGFR1, BCAR3, GSN, TLR9, TTN, |
| | | | | RAP1A, RAC3, RHOQ, GAB1, PTPN11, PLCG2, |
| | | | | PIK3R6, ACTN4, TSPAN6, FYN, ARPC1B, |
| | | | | PIK3R5, CRK, ITGB8, BCAR1, ACTR3, |
| | | | | PPP1R12A, PFN4, PIK3C2A, GRB2, ACTN2, |
| | | | | ITGAL, GIT1, ITGB2, LIMS1, ITGAX |
| FcγR-mediated | 3.05 | 0.28 | −3.922 | FYN, ARPC1B, CRK, FCGR1A, PTEN, PAK1, |
| Phagocytosis in | | | | ACTR3, MAPK3, EZR, PIK3CG, HCK, ACTG2, |
| Macs and Monos | | | | FCGR3A/FCGR3B, FGR, PRKCA, VAV2, ACTR2, |
| | | | | RPS6KB1, FCGR2A, RAC3, PLD4, NCF1, SYK, |
| | | | | PRKCD, LYN, VAV1 |
| PI3K Signaling in | 5.04 | 0.3 | −3.667 | BLNK, CD81, FYN, RAF1, Calm1 (includes |
| B Lymphocytes | | | | others), PDIA3, NFKBIE, ATF6, KRAS, NFKB1, |
| | | | | PTEN, BLK, PLCD3, IKBKB, IKBKG, JUN, |
| | | | | PIK3CG, MAPK3, PPP3CA, CAMK2B, VAV2, |
| | | | | IL4R, CALML5, C3, NFKB2, MALT1, BTK, TLR4, |
| | | | | CD40, CD180, PLCG2, SYK, SH2B2, LYN, VAV1, |
| | | | | PIK3AP1, PLEKHA2, ELK1, CAMK2G |
| NF-κB Activation | 3.96 | 0.31 | −3.657 | RAF1, NFKBIE, PIK3R5, KRAS, NFKB1, ITGB3, |
| | | | | IKBKB, ITGA3, IKBKG, MAPK3, PIK3CG, |
| | | | | ITGAV, PRKCA, MAP3K14, PIK3C2A, GRB2, |
| | | | | FGFR1, NFKB2, TLR9, ITGAL, ITGB2, RIPK1, |
| | | | | GAB1, PTPN11, PRKCD, PIK3R6, EIF2AK2 |
| IL-8 Signaling | 2.69 | 0.228 | −3.507 | RAF1, ICAM1, GNB5, PIK3R5, EGF, VEGFB, |
| | | | | KRAS, NFKB1, IQGAP1, CCND1, GNG7, ITGB3, |
| | | | | IKBKB, IKBKG, GNG11, JUN, RHOB, PIK3CG, |
| | | | | MAPK3, CYBB, ITGAV, GNA13, LASP1, PRKCA, |
| | | | | RPS6KB1, PIK3C2A, GRB2, FGFR1, VEGFC, |
| | | | | IRAK3, MAPK12, TLR9, RAC3, PLD4, ITGB2, |
| | | | | MPO, CCND2, RHOQ, ARAF, GAB1, PTPN11, |
| | | | | PRKCD, PIK3R6, IRAK4, ITGAX |
| Production of NO | 3.65 | 0.247 | −3.429 | MAP3K15, MAP3K11, APOF, NFKBIE, PIK3R5, |
| and ROS in | | | | MAPK13, MAP3K5, NFKB1, IKBKB, LYZ, |
| Macrophages | | | | IKBKG, JUN, RHOB, PPP1R12A, PPM1J, |
| | | | | PIK3CG, MAPK3, HOXA10, CYBB, NOS2, |
| | | | | TNFRSF1B, PRKCA, MAP3K14, MAP2K7, |
| | | | | PTPN6, APOM, PIK3C2A, GRB2, PPP2R5D, |
| | | | | FGFR1, NFKB2, PCYOX1, MAPK12, TLR9, |
| | | | | RAP1A, MAP3K12, TLR4, NCF1, MPO, RHOQ, |
| | | | | GAB1, PTPN11, PRKCD, PLCG2, PIK3R6, |
| | | | | MAP3K8, IRF8, MAP3K3 |
| PKCθ Signaling in | 4.27 | 0.27 | −3.333 | MAP3K15, FYN, CACNA2D2, MAP3K11, HLA-A, |
| T Lymphocytes | | | | NFKBIE, CACNA1H, PIK3R5, KRAS, MAP3K5, |
| | | | | NFKB1, CACNA1F, IKBKB, IKBKG, JUN, |
| | | | | PIK3CG, MAPK3, CACNG8, PPP3CA, CAMK2B, |
| | | | | VAV2, MAP3K14, CACNB1, PIK3C2A, GRB2, |
| | | | | FGFR1, CACNA1C, NFKB2, MALT1, TLR9, |
| | | | | RAC3, CACNA1A, MAP3K12, GAB1, PTPN11, |
| | | | | H2-Eb2, PLCG2, PIK3R6, HLA-DOB, VAV1, |
| | | | | MAP3K8, MAP3K3, CAMK2G |
| Role of RIG1-like | 1.61 | 0.273 | −3.317 | IFIH1, TANK, IKBKB, TRAF3, IRF7, IKBKG, |
| Receptors in | | | | RIPK1, NFKBIE, DDX58, NFKB2, CASP8, |
| Antiviral Innate | | | | NFKB1 |
| Immunity | | | | |
| NGF Signaling | 3.5 | 0.273 | −3.307 | MAP3K15, RAF1, MAP3K11, PIK3R5, CRK, |
| | | | | KRAS, MAP3K5, NFKB1, IKBKB, IKBKG, |
| | | | | PIK3CG, MAPK3, RPS6KA2, SMPD3, MAP3K14, |
| | | | | RPS6KB1, PIK3C2A, GRB2, FGFR1, NFKB2, |
| | | | | TLR9, MAPK12, RAP1A, MAP3K12, RPS6KA6, |
| | | | | PTPN11, GAB1, PRKCD, PLCG2, PIK3R6, |
| | | | | MAP3K8, ELK1, MAP3K3 |
| PRRs in | 4.1 | 0.277 | −3.286 | PIK3R5, TLR8, EIF2S1, NFKB1, IFIH1, PIK3CG, |
| Recognition of | | | | MAPK3, TLR7, OSM, CLEC6A, PRKCA, OAS1, |
| Bacteria and | | | | NLRP3, C3, C5AR1, OAS2, PIK3C2A, IL10, |
| Viruses | | | | MYD88, GRB2, FGFR1, NFKB2, MAPK12, TLR9, |
| | | | | OAS3, TLR4, CLEC7A, NOD2, IRF7, GAB1, |
| | | | | PTPN11, IL12B, PRKCD, PLCG2, SYK, DDX58, |
| | | | | PIK3R6, EIF2AK2 |
| Inflammasome | 3.62 | 0.5 | −3.162 | TLR4, NOD2, NLRP3, MYD88, CTSB, NEK7, |
| pathway | | | | NFKB2, CASP8, PANX1, NFKB1 |
| B Cell Receptor | 6.88 | 0.297 | −3.101 | RAF1, MAP3K15, MAP3K11, KRAS, MAPK13, |
| Signaling | | | | BCL6, PTEN, IKBKB, IKBKG, PIK3CG, MAPK3, |
| | | | | MAP3K14, RPS6KB1, PTPN6, FGFR1, MALT1, |
| | | | | NFKB2, MAPK12, TLR9, RAP1A, MAP3K12, |
| | | | | GAB1, PTPN11, INPP5F, PLCG2, SYK, PIK3R6, |
| | | | | VAV1, PIK3AP1, INPP5K, CAMK2G, MAP2K6, |
| | | | | BLNK, Calm1 (includes others), NFKBIE, |
| | | | | PIK3R5, IGHG1, MAP3K5, NFKB1, OCRL, JUN, |
| | | | | CD22, PPP3CA, CAMK2B, VAV2, MAP2K7, |
| | | | | CALML5, PIK3C2A, FCGR2A, GRB2, BTK, |
| | | | | INPP5J, LYN, MEF2C, MAP3K8, ELK1, MAP3K3 |
| Rac Signaling | 3.79 | 0.282 | −2.959 | ABI2, RAF1, MAP3K11, ARPC1B, PIK3R5, |
| | | | | KRAS, PIP5K1B, IQGAP1, NFKB1, ITGA3, |
| | | | | PAK1, ACTR3, JUN, CYFIP2, PIK3CG, MAPK3, |
| | | | | CYBB, ACTR2, RPS6KB1, TIAM1, MAP2K7, |
| | | | | PIK3C2A, GRB2, FGFR1, NFKB2, TLR9, GAB1, |
| | | | | PTPN11, CYFIP1, PIP5K1C, CD44, PIK3R6, |
| | | | | ELK1 |
| Th1 Pathway | 3.22 | 0.259 | −2.921 | MAP2K6, SOCS3, CD40LG, ICAM1, HLA-A, |
| | | | | KLRD1, PIK3R5, NCSTN, CD8A, NFKB1, NFIL3, |
| | | | | PIK3CG, KLRC1, DLL1, TNFSF11, NOTCH3, |
| | | | | PIK3C2A, IL10, GRB2, FGFR1, IL27, STAT3, |
| | | | | TLR9, ITGB2, CD40, GAB1, PTPN11, IL12B, H2- |
| | | | | Eb2, ICOS, PIK3R6, HLA-DOB, VAV1, NOTCH1, |
| | | | | PSEN1 |
| NF-κB Signaling | 6.63 | 0.298 | −2.885 | RAF1, CSNK2A1, TRAF3, CD40LG, TGFBR1, |
| | | | | TGFBR3, TLR8, KRAS, TNFRSF17, IL1R2, |
| | | | | TGFBR2, IKBKB, TNIP1, IKBKG, PIK3CG, |
| | | | | LTBR, TAB1, MAP3K14, FGFR1, NFKB2, |
| | | | | MALT1, TLR9, ARAF, GAB1, CD40, PTPN11, |
| | | | | PLCG2, PIK3R6, FGFRL1, MAP2K6, NFKBIE, |
| | | | | PIK3R5, TNFAIP3, EGF, NFKB1, TANK, TLR7, |
| | | | | CASP8, TNFRSF1B, TNFSF11, MAP2K7, |
| | | | | PIK3C2A, MYD88, GRB2, IRAK3, IL1R1, TLR4, |
| | | | | RIPK1, MAP3K8, BTRC, EIF2AK2, TIRAP, |
| | | | | MAP3K3, IRAK4 |
| Dendritic Cell | 3.65 | 0.247 | −2.832 | CD40LG, ICAM1, PDIA3, HLA-A, LEPR, |
| Maturation | | | | NFKBIE, PIK3R5, CD83, MAPK13, IGHG1, |
| | | | | NFKB1, FCGR1A, PLCD3, IKBKB, CD1D, |
| | | | | IKBKG, IL1RL2, PIK3CG, MAPK3, LTBR, |
| | | | | COL11A2, TNFRSF1B, FCGR3A/FCGR3B, TAB1, |
| | | | | MAP3K14, PIK3C2A, IL10, FCGR2A, MYD88, |
| | | | | GRB2, FGFR1, IL15, NFKB2, MAPK12, TLR9, |
| | | | | TLR4, Cd1d2, CD40, GAB1, PTPN11, IL12B, H2- |
| | | | | Eb2, PLCG2, PIK3R6, HLA-DOB, STAT2, IRF8, |
| | | | | COL3A1 |
| Signaling by Rho | 3.63 | 0.234 | −2.774 | RAF1, MAP3K11, CDH22, CDH24, GNB5, |
| Family GTPases | | | | CDC42EP2, PIP5K1B, PAK1, ITGA3, RHOB, |
| | | | | PIK3CG, EZR, MAPK3, CYBB, GNA13, ACTG2, |
| | | | | ACTR2, FGFR1, ARHGEF15, SEPT7, NFKB2, |
| | | | | GNAZ, MAPK12, TLR9, PKN1, MAP3K12, |
| | | | | RHOQ, CDH5, GAB1, PTPN11, PIP5K1C, |
| | | | | CYFIP1, PIK3R6, ARHGEF18, ARHGEF9, |
| | | | | GNAL, ARPC1B, SEPT3, PIK3R5, CDH23, |
| | | | | SEPT11, GNA14, NFKB1, IQGAP1, GNG7, |
| | | | | CDH11, ACTR3, GNG11, JUN, PPPIR12A, |
| | | | | ARHGEF3, MAP2K7, PIK3C2A, SEPT4, GRB2, |
| | | | | MYLPF, SEPT1, ELK1, MSN |
| HGF Signaling | 5.6 | 0.322 | −2.667 | CDKN2A, MAP3K15, RAF1, RAPGEF1, |
| | | | | MAP3K11, PIK3R5, KRAS, MAP3K5, CCND1, |
| | | | | PAK1, ITGA3, JUN, HGF, PIK3CG, MAPK3, |
| | | | | PRKCA, MAP3K14, MAP2K7, PIK3C2A, GRB2, |
| | | | | CRKL, FGFR1, STAT3, MAPK12, TLR9, RAP1A, |
| | | | | ELF1, MAP3K12, GAB1, PTPN11, PRKCD, |
| | | | | PLCG2, CDKN1A, PIK3R6, MAP3K8, ELK1, |
| | | | | MAP3K3 |
| Actin Cytoskeleton | 2.14 | 0.211 | −2.655 | ABI2, RAF1, FGD3, ARPC1B, PDGFA, PIK3R5, |
| Signaling | | | | EGF, KRAS, CRN PIP5K1B, IQGAP1, BCAR1, |
| | | | | FGF13, PAK1, ITGA3, ACTR3, CYFIP2, |
| | | | | PPPIRI2A, FLNA, PIK3CG, EZR, MAPK3, |
| | | | | GNA13, ACTG2, MATK, VAV2, ACTR2, TIAM1, |
| | | | | PIK3C2A, GRB2, MYLPF, CRKL, FGFR1, |
| | | | | ACTN2, GSN, TLR9, TTN, RAC3, GIT1, TIAM2, |
| | | | | GAB1, PTPN11, CYFIP1, PIP5K1C, PIK3R6, |
| | | | | VAV1, ACTN4, MSN |
| PDGF Signaling | 4.11 | 0.311 | −2.646 | RAF1, CSNK2A1, PDGFA, PIK3R5, CRK, KRAS, |
| | | | | OCRL, MYC, JUN, MAPK3, PIK3CG, SPHK1, |
| | | | | PRKCA, PIK3C2A, GRB2, FGFR1, CRKL, STAT3, |
| | | | | TLR9, GAB1, PTPN11, INPP5J, INPP5F, PLCG2, |
| | | | | PIK3R6, EIF2AK2, INPP5K, ELK1 |
| CXCR4 Signaling | 1.33 | 0.2 | −2.646 | RAF1, GNB5, PIK3R5, CRK, KRAS, GNA14, |
| | | | | BCAR1, GNG7, PAK1, GNG11, JUN, RHOB, |
| | | | | PIK3CG, MAPK3, GNA13, PRKCA, PIK3C2A, |
| | | | | GRB2, MYLPF, FGFR1, ADCY6, GNAZ, TLR9, |
| | | | | MAPK12, RHOQ, GAB1, PTPN11, PRKCD, LYN, |
| | | | | PIK3R6, ELK1, ELMO2, GNAL |
| Macropinocytosis | 4.11 | 0.321 | −2.524 | PDGFA, PIK3R5, EGF, KRAS, ITGB8, ITGB3, |
| Signaling | | | | PAK1, RAB5A, HGF, PIK3CG, PRKCA, PIK3C2A, |
| | | | | GRB2, FGFR1, TLR9, RAB34, CSF1R, ITGB2, |
| | | | | ABI1, PTPN11, GAB1, CSF1, PLCG2, PRKCD, |
| | | | | PIK3R6, ACTN4 |
| p70S6K Signaling | 1.71 | 0.22 | −2.502 | RAF1, YWHAH, PDIA3, PIK3R5, KRAS, PLCD3, |
| | | | | MAPK3, PIK3CG, PPM1J, EEF2K, PRKCA, |
| | | | | RPS6KB1, IL4R, YWHAG, PIK3C2A, GRB2, |
| | | | | PPP2R5D, FGFR1, TLR9, BTK, PTPN11, GAB1, |
| | | | | MAPT, SYK, PLCG2, PRKCD, LYN, PIK3R6, |
| | | | | AGTR1 |
| FcγRIIB Signaling | 3.03 | 0.291 | −2.5 | BLNK, CACNB1, CACNA2D2, PIK3C2A, GRB2, |
| in B Lymphocytes | | | | FGFR1, CACNA1H, PIK3R5, CACNA1C, KRAS, |
| | | | | MAPK12, TLR9, CACNA1A, CACNA1F, BTK, |
| | | | | PTPN11, GAB1, SYK, PIK3CG, PLCG2, LYN, |
| | | | | PIK3R6, CACNG8 |
| Granzyme B | 1.64 | 0.375 | −2.449 | CASP9, APAF1, BID, CASP8, LMNB1, PARP1 |
| Signaling | | | | |
| fMLP Signaling in | 3.02 | 0.26 | −2.414 | RAF1, Calm1 (includes others), ARPC1B, |
| Neutrophils | | | | NFKBIE, GNB5, PIK3R5, KRAS, NFKB1, GNG7, |
| | | | | IKBKG, ACTR3, GNG11, PIK3CG, MAPK3, |
| | | | | CYBB, PPP3CA, PRKCA, ACTR2, CALML5, |
| | | | | PIK3C2A, GRB2, FGFR1, FPR2, NFKB2, TLR9, |
| | | | | FPR1, NCF1, GAB1, PTPN11, PRKCD, PIK3R6, |
| | | | | ELK1 |
| Toll-like Receptor | 3.29 | 0.303 | −2.357 | MAP2K6, MAP3K14, TICAM2, MYD88, TLR8, |
| Signaling | | | | TNFAIP3, MAPK13, NFKB2, IRAK3, NFKB1, |
| | | | | MAPK12, TLR9, TLR4, IKBKB, IKBKG, JUN, |
| | | | | IL12B, TLR7, EIF2AK2, TIRAP, ELK1, TAB1, |
| | | | | IRAK4 |
| Thrombopoietin | 2.24 | 0.277 | −2.357 | RAF1, P1K3C2A, GRB2, FGFR1, PIK3R5, KRAS, |
| Signaling | | | | STAT3, TLR9, MYC, JUN, PTPN11, GAB1, |
| | | | | MAPK3, PRKCD, PLCG2, PIK3CG, PIK3R6, |
| | | | | PRKCA |
| Glioma | 1.89 | 0.257 | −2.357 | TIMP3, P1K3C2A, GRB2, FGFR1, HMMR, |
| Invasiveness | | | | PIK3R5, KRAS, TLR9, ITGB3, RHOQ, RHOB, |
| Signaling | | | | PTPN11, GAB1, MAPK3, PIK3CG, PIK3R6, |
| | | | | ITGAV, CD44 |
| Renal Cell | 1.73 | 0.241 | −2.357 | RAPGEF1, RAF1, P1K3C2A, GRB2, FGFR1, |
| Carcinoma | | | | PIK3R5, CRK, KRAS, HIF1A, TLR9, RAP1A, |
| Signaling | | | | PAK1, JUN, PTPN11, GAB1, CUL2, MAPK3, |
| | | | | HGF, PIK3CG, PIK3R6 |
| 14-3-3-mediated | 2.27 | 0.237 | −2.353 | RAF1, YWHAH, PDIA3, TP73, PIK3R5, KRAS, |
| Signaling | | | | MAP3K5, PLCD3, JUN, MAPK3, PIK3CG, |
| | | | | TUBB4A, PRKCA, TUBB1, TUBB3, YWHAG, |
| | | | | PIK3C2A, GRB2, FGFR1, TUBA4A, TLR9, |
| | | | | MAPK12, GAB1, TUBB6, PTPN11, PRKCD, |
| | | | | MAPT, PLCG2, PIK3R6, CDKN1B, ELK1 |
| PEDF Signaling | 2.78 | 0.276 | −2.294 | RAF1, PIK3C2A, GRB2, NFKBIE, FGFR1, |
| | | | | SERPINF1, PIK3R5, KRAS, MAPK13, NFKB2, |
| | | | | NFKB1, MAPK12, TLR9, HNF1B, IKBKB, |
| | | | | IKBKG, PTPN11, GAB1, MAPK3, PIK3CG, |
| | | | | PIK3R6, CFLAR, CASP8, ELK1 |
| GP6 Signaling | 4.33 | 0.284 | −2.271 | FYN, Calm1 (includes others), COL4A5, PIK3R5, |
| Pathway | | | | COL4A2, Col17a1, Col6a4, ITGB3, COL5A1, |
| | | | | LAMC1, PIK3CG, LAMA1, COL11A2, KLF12, |
| | | | | COL27A1, PRKCA, COL5A2, CALML5, COL4A1, |
| | | | | PIK3C2A, GRB2, FGFR1, LAMC3, COL20A1, |
| | | | | TLR9, BTK, COL13A1, GAB1, PTPN11, PRKCD, |
| | | | | PLCG2, SYK, LYN, PIK3R6, ADAM10, COL4A4, |
| | | | | COL7A1, COL3A1 |
| Insulin Receptor | 2.57 | 0.241 | −2.263 | RAF1, SOCS3, FYN, RAPGEF1, SGK1, PIK3R5, |
| Signaling | | | | CRK, KRAS, STXBP4, OCRL, PRKAG1, PTEN, |
| | | | | SCNN1A, PPP1R12A, PIK3CG, MAPK3, PTPN1, |
| | | | | RPS6KB1, PIK3C2A, GRB2, CRKL, FGFR1, |
| | | | | ACLY, TLR9, GRB10, RHOQ, PRKAR2B, GAB1, |
| | | | | PTPN11, INPP5J, INPP5F, SH2B2, PIK3R6, |
| | | | | INPP5K |
| Lymphotoxin β | 3.28 | 0.313 | −2.236 | MAP3K14, TRAF3, PIK3C2A, GRB2, FGFR1, |
| Receptor Signaling | | | | APAF1, PIK3R5, NFKB2, NFKB1, TLR9, |
| | | | | DIABLO, IKBKB, IKBKG, CASP9, PTPN11, |
| | | | | GAB1, MAPK3, PIK3CG, PIK3R6, LTBR, BIRC2 |
| Activation of IRF | 3.23 | 0.317 | −2.236 | TRAF3, IL10, NFKBIE, ZBP1, IRF9, NFKB2, |
| by Cytosolic | | | | ADAR, NFKB1, MAPK12, ISG15, IFIH1, TANK, |
| Pattern | | | | IKBKB, IRF7, IKBKG, RIPK1, JUN, CD40, |
| Recognition | | | | DDX58, STAT2 |
| Receptors | | | | |
| GM-CSF Signaling | 2.74 | 0.288 | −2.236 | RAF1, PIK3C2A, GRB2, FGFR1, PIK3R5, KRAS, |
| | | | | STAT3, TLR9, CCND1, CSF2RB, PTPN11, GAB1, |
| | | | | MAPK3, PIK3CG, LYN, HCK, PIK3R6, ELK1, |
| | | | | PPP3CA, CAMK2G, CAMK2B |
| Prolactin Signaling | 2.04 | 0.253 | −2.236 | FYN, SOCS3, RAF1, PIK3C2A, GRB2, FGFR1, |
| | | | | PIK3R5, KRAS, CEBPB, STAT3, TLR9, MYC, |
| | | | | JUN, PTPN11, GAB1, MAPK3, PRKCD, PLCG2, |
| | | | | PIK3CG, PIK3R6, PRKCA |
| IL-3 Signaling | 3.49 | 0.301 | −2.2 | RAPGEF1, RAF1, PIK3R5, KRAS, PAK1, JUN, |
| | | | | MAPK3, PIK3CG, PPP3CA, PRKCA, STAT6, |
| | | | | PTPN6, PIK3C2A, IL3RA, GRB2, FGFR1, CRKL, |
| | | | | STAT3, TLR9, CSF2RB, PTPN11, GAB1, PRKCD, |
| | | | | PIK3R6, ELK1 |
| RANK Signaling | 6.56 | 0.353 | −2.197 | MAP2K6, MAP3K15, RAF1, Calm1 (includes |
| in Osteoclasts | | | | others), MAP3K11, NFKBIE, PIK3R5, MAPK13, |
| | | | | MAP3K5, NFKB1, IKBKB, IKBKG, JUN, |
| | | | | PIK3CG, MAPK3, PPP3CA, MAP3K14, |
| | | | | CALML5, MAP2K7, TNFSF11, PIK3C2A, GRB2, |
| | | | | FGFR1, NFKB2, GSN, TLR9, MAPK12, XIAP, |
| | | | | MAP3K12, GAB1, PTPN11, PIK3R6, MAP3K8, |
| | | | | ELK1, MAP3K3, BIRC2 |
| Thrombin | 1.95 | 0.211 | −2.197 | RAF1, GATA1, PDIA3, GNB5, PIK3R5, EGF, |
| Signaling | | | | KRAS, MAPK13, GNA14, NFKB1, GNG7, |
| | | | | PLCD3, IKBKB, GNG11, RHOB, PPP1R12A, |
| | | | | PIK3CG, MAPK3, GNA13, ARHGEF3, PRKCA, |
| | | | | CAMK2B, RPS6KB1, PIK3C2A, GRB2, MYLPF, |
| | | | | ARHGEF15, FGFR1, ADCY6, NFKB2, GNAZ, |
| | | | | MAPK12, TLR9, RHOQ, GAB1, PTPN11, |
| | | | | PRKCD, PLCG2, PIK3R6, ELK1, ARHGEF9, |
| | | | | GNAL, CAMK2G |
| Th2 Pathway | 2.93 | 0.247 | −2.191 | SOCS3, TNFSF4, ICAM1, TNFRSF4, TGFBR1, |
| | | | | HLA-A, TGFBR3, PIK3R5, NCSTN, NFKB1, |
| | | | | TGFBR2, JUN, PIK3CG, CCR8, STAT6, IL4R, |
| | | | | DLL1, NOTCH3, PIK3C2A, IL10, GRB2, FGFR1, |
| | | | | ACVR1, TLR9, ITGB2, CD40, GAB1, PTPN11, |
| | | | | IL12B, H2-Eb2, ICOS, PIK3R6, CXCR6, HLA- |
| | | | | DOB, VAV1, NOTCH1, PSEN1 |
| ErbB4 Signaling | 2.09 | 0.264 | −2.183 | RAF1, ADAM17, NRG2, PIK3C2A, GRB2, |
| | | | | FGFR1, PIK3R5, NCSTN, KRAS, TLR9, PTPN11, |
| | | | | GAB1, MAPK3, PRKCD, PLCG2, PIK3CG, |
| | | | | PIK3R6, PSEN1, PRKCA |
| CNTF Signaling | 1.96 | 0.266 | −2.183 | RPS6KB1, RAF1, PIK3C2A, GRB2, FGFR1, |
| | | | | PIK3R5, KRAS, STAT3, TLR9, LIFR, RPS6KA6, |
| | | | | PTPN11, GAB1, MAPK3, PIK3CG, PIK3R6, |
| | | | | RPS6KA2 |
| Estrogen- | 1.61 | 0.238 | −2.183 | PIK3C2A, GRB2, FGFR1, PIK3R5, KRAS, |
| Dependent Breast | | | | NFKB2, NFKB1, TLR9, CCND1, Akr1b7, JUN, |
| Cancer Signaling | | | | PTPN11, GAB1, MAPK3, PIK3CG, PIK3R6, |
| | | | | ELK1, ESR1, HSD17B14 |
| iNOS Signaling | 3.3 | 0.356 | −2.138 | CALML5, Calm1 (includes others), MYD88, |
| | | | | NFKBIE, MAPK13, IRAK3, NFKB2, NFKB1, |
| | | | | MAPK12, IKBKB, TLR4, IKBKG, JUN, NOS2, |
| | | | | TAB1, IRAK4 |
| IL-2 Signaling | 1.62 | 0.25 | −2.138 | RAF1, CSNK2A1, PIK3C2A, GRB2, FGFR1, |
| | | | | PIK3R5, KRAS, TLR9, JUN, PTPN11, GAB1, |
| | | | | MAPK3, PIK3CG, SYK, PIK3R6, ELK1 |
| Notch Signaling | 1.32 | 0.263 | −2.121 | FURIN, DLL1, ADAM17, NOTCH3, DTX3, |
| | | | | NCSTN, HES1, NOTCH1, PSEN1, RFNG |
| Pancreatic | 5.55 | 0.317 | −2.117 | CDKN2A, RAF1, TGFBR1, PA2G4, PIK3R5, EGF, |
| Adenocarcinoma | | | | VEGFB, KRAS, E2F3, NFKB1, CDKN2B, |
| Signaling | | | | CCND1, RAD51, TGFBR2, CASP9, PIK3CG, |
| | | | | MAPK3, RALGDS, E2F2, TFDP1, PIK3C2A, |
| | | | | GRB2, FGFR1, VEGFC, MDM2, NFKB2, STAT3, |
| | | | | TLR9, MAPK12, PLD4, PTPN11, GAB1, E2F7, |
| | | | | CDKN1A, PIK3R6, CDKN1B, ELK1, NOTCH1 |
| LPS-stimulated | 4.4 | 0.322 | −2.117 | MAP2K6, RAF1, NFKBIE, PIK3R5, KRAS, |
| MAPK Signaling | | | | MAPK13, MAP3K5, NFKB1, IKBKB, IKBKG, |
| | | | | PAK1, JUN, MAPK3, PIK3CG, PRKCA, |
| | | | | MAP3K14, PIK3C2A, GRB2, FGFR1, NFKB2, |
| | | | | TLR9, MAPK12, TLR4, PTPN11, GAB1, PRKCD, |
| | | | | PIK3R6, ELK1 |
| Type II Diabetes | 3.3 | 0.253 | −2.117 | SOCS3, CACNA2D2, PRKAB2, NFKBIE, |
| Mellitus Signaling | | | | CACNA1H, PIK3R5, MAP3K5, NFKB1, PRKAG1, |
| | | | | CACNA1F, IKBKB, IKBKG, ACSBG1, PIK3CG, |
| | | | | MAPK3, PRKAA2, CACNG8, TNFRSF1B, |
| | | | | SMPD3, PRKCA, MAP3K14, CACNB1, MAP2K7, |
| | | | | PIK3C2A, GRB2, FGFR1, CACNA1C, CEBPB, |
| | | | | NFKB2, TLR9, MAPK12, CACNA1A, GAB1, |
| | | | | PTPN11, PRKCD, SH2B2, PIK3R6, SLC27A6, |
| | | | | SLC27A1 |
| Gα12/13 Signaling | 3.22 | 0.259 | −2.058 | RAF1, NFKBIE, CDH22, TBXA2R, CDH24, |
| | | | | PIK3R5, CDH23, KRAS, MAP3K5, NFKB1, |
| | | | | CDH11, IKBKB, IKBKG, JUN, PIK3CG, MAPK3, |
| | | | | GNA13, VAV2, MAP2K7, PIK3C2A, GRB2, |
| | | | | MYLPF, FGFR1, NFKB2, TLR9, MAPK12, BTK, |
| | | | | LPAR6, CDH5, GAB1, PTPN11, PIK3R6, MEF2C, |
| | | | | VAV1, ELK1 |
| SAPK/JNK | 3.69 | 0.288 | −2.043 | MAP3K11, PIK3R5, CRK, KRAS, MAP3K5, |
| Signaling | | | | GNG7, GNG11, JUN, GADD45A, PIK3CG, |
| | | | | GNA13, TAB1, MAP2K7, PIK3C2A, GRB2, |
| | | | | FGFR1, CRKL, MAPK12, TLR9, MAPK8IP1, |
| | | | | RAC3, DAXX, MAP3K12, RIPK1, GAB1, PTPN11, |
| | | | | PIK3R6, DUSP4, ELK1, MAP3K3 |
| PI3K/AKT | 2.59 | 0.248 | −2.043 | RAF1, YWHAH, NFKBIE, KRAS, MAP3K5, |
| Signaling | | | | NFKB1, CCND1, OCRL, PTEN, IKBKB, IKBKG, |
| | | | | ITGA3, HSP90B1, PPM1J, MAPK3, PIK3CG, |
| | | | | RPS6KB1, YWHAG, GRB2, PPP2R5D, MDM2, |
| | | | | NFKB2, MAPK8IP1, GAB1, INPP5J, INPP5F, |
| | | | | LIMS1, CDKN1A, MAP3K8, CDKN1B, INPP5K |
| JAK/Stat Signaling | 3.09 | 0.289 | −2.041 | STAT6, SOCS3, RAF1, PTPN6, PIK3C2A, GRB2, |
| | | | | FGFR1, PIK3R5, KRAS, CEBPB, STAT3, NFKB2, |
| | | | | NFKB1, TLR9, PIAS3, JUN, PTPN11, GAB1, |
| | | | | MAPK3, PIK3CG, CDKN1A, PTPN1, PIK3R6, |
| | | | | STAT2 |

TABLE 63
GO Biological Pathway Terms by P Value for Transcripts Increased by HDAC6 Inhibitor Treatment

| | | | Anno- | Signif- | Ex- | Neg Log | GO | Re- |
|---|---|---|---|---|---|---|---|---|
| GO Term | Term/Pathway | p-value | tated | icant | pected | (p-value) | type | sponse |
| GO:0042384 | cilium assembly | 3.25E−07 | 153 | 35 | 12.5 | 6.49E+00 | BP | UP |
| GO:0055114 | oxidation-reduction | 3.53E−06 | 946 | 117 | 77.27 | 5.45E+00 | BP | UP |
| | process | | | | | | | |
| GO:0006750 | glutathione | 2.65E−05 | 13 | 7 | 1.06 | 4.58E+00 | BP | UP |
| | biosynthetic process | | | | | | | |
| GO:0006749 | glutathione | 3.62E−05 | 46 | 18 | 3.76 | 4.44E+00 | BP | UP |
| | metabolic process | | | | | | | |
| GO:0046686 | response to cadmium | 9.23E−05 | 36 | 11 | 2.94 | 4.03E+00 | BP | UP |
| | ion | | | | | | | |
| GO:0043113 | receptor clustering | 0.000151492 | 44 | 12 | 3.59 | 3.82E+00 | BP | UP |
| GO:0061512 | protein localization | 0.000219761 | 22 | 8 | 1.8 | 3.66E+00 | BP | UP |
| | to cilium | | | | | | | |
| GO:0006782 | protoporphyrinogen | 0.000344241 | 9 | 5 | 0.74 | 3.46E+00 | BP | UP |
| | IX biosynthetic | | | | | | | |
| | process | | | | | | | |
| GO:0006552 | leucine catabolic | 0.00054405 | 3 | 3 | 0.25 | 3.26E+00 | BP | UP |
| | process | | | | | | | |
| GO:0006068 | ethanol catabolic | 0.000581648 | 6 | 4 | 0.49 | 3.24E+00 | BP | UP |
| | process | | | | | | | |
| GO:0006534 | cysteine metabolic | 0.000642137 | 10 | 5 | 0.82 | 3.19E+00 | BP | UP |
| | process | | | | | | | |
| GO:0035721 | intraciliary | 0.000642137 | 10 | 5 | 0.82 | 3.19E+00 | BP | UP |
| | retrograde transport | | | | | | | |
| GO:0044281 | small molecule | 0.001175761 | 1644 | 195 | 134.28 | 2.93E+00 | BP | UP |
| | metabolic process | | | | | | | |
| GO:0035735 | intraciliary | 0.001269198 | 7 | 4 | 0.57 | 2.90E+00 | BP | UP |
| | transport involved | | | | | | | |
| | in cilium | | | | | | | |
| | morphogenesis | | | | | | | |
| GO:0006783 | heme biosynthetic | 0.001736561 | 21 | 10 | 1.72 | 2.76E+00 | BP | UP |
| | process | | | | | | | |
| GO:2000649 | regulation of | 0.001966232 | 36 | 9 | 2.94 | 2.71E+00 | BP | UP |
| | sodium ion | | | | | | | |
| | transmembrane | | | | | | | |
| | transporter activity | | | | | | | |
| GO:0006083 | acetate metabolic | 0.002043103 | 4 | 3 | 0.33 | 2.69E+00 | BP | UP |
| | process | | | | | | | |
| GO:0018916 | nitrobenzene | 0.002043103 | 4 | 3 | 0.33 | 2.69E+00 | BP | UP |
| | metabolic process | | | | | | | |
| GO:0045724 | positive regulation | 0.002043103 | 4 | 3 | 0.33 | 2.69E+00 | BP | UP |
| | of cilium assembly | | | | | | | |
| GO:0006677 | glycosylceramide | 0.002663323 | 13 | 5 | 1.06 | 2.57E+00 | BP | UP |
| | metabolic process | | | | | | | |
| GO:0042219 | cellular modified | 0.002663323 | 13 | 5 | 1.06 | 2.57E+00 | BP | UP |
| | amino acid | | | | | | | |
| | catabolic process | | | | | | | |
| GO:0046689 | response to | 0.002663323 | 13 | 5 | 1.06 | 2.57E+00 | BP | UP |
| | mercury ion | | | | | | | |
| GO:0043001 | Golgi to plasma | 0.003465353 | 32 | 8 | 2.61 | 2.46E+00 | BP | UP |
| | membrane protein | | | | | | | |
| | transport | | | | | | | |
| GO:1901017 | negative regulation | 0.003866549 | 14 | 5 | 1.14 | 2.41E+00 | BP | UP |
| | of potassium ion | | | | | | | |
| | transmembrane | | | | | | | |
| | transporter activity | | | | | | | |
| GO:0043648 | dicarboxylic acid | 0.004489029 | 79 | 14 | 6.45 | 2.35E+00 | BP | UP |
| | metabolic process | | | | | | | |
| GO:0060632 | regulation of | 0.004770823 | 15 | 7 | 1.23 | 2.32E+00 | BP | UP |
| | microtubule- | | | | | | | |
| | based movement | | | | | | | |
| GO:0048069 | eye pigmentation | 0.004796711 | 5 | 3 | 0.41 | 2.32E+00 | BP | UP |
| GO:0071918 | urea transmembrane | 0.004796711 | 5 | 3 | 0.41 | 2.32E+00 | BP | UP |
| | transport | | | | | | | |
| GO:0046688 | response to | 0.004952292 | 27 | 7 | 2.21 | 2.31E+00 | BP | UP |
| | copper ion | | | | | | | |
| GO:0006833 | water transport | 0.005413787 | 15 | 5 | 1.23 | 2.27E+00 | BP | UP |
| GO:0006979 | response to | 0.005470968 | 353 | 43 | 28.83 | 2.26E+00 | BP | UP |
| | oxidative stress | | | | | | | |
| GO:0050808 | synapse organization | 0.006053111 | 189 | 26 | 15.44 | 2.22E+00 | BP | UP |
| GO:0051260 | protein | 0.006310122 | 247 | 32 | 20.17 | 2.20E+00 | BP | UP |
| | homooligomerization | | | | | | | |
| GO:0002223 | stimulatory C-type | 0.006667992 | 2 | 2 | 0.16 | 2.18E+00 | BP | UP |
| | lectin receptor | | | | | | | |
| | signaling pathway | | | | | | | |
| GO:0007181 | transforming | 0.006667992 | 2 | 2 | 0.16 | 2.18E+00 | BP | UP |
| | growth factor beta | | | | | | | |
| | receptor complex | | | | | | | |
| | assembly | | | | | | | |
| GO:0018283 | iron incorporation | 0.006667992 | 2 | 2 | 0.16 | 2.18E+00 | BP | UP |
| | into metallo- | | | | | | | |
| | sulfur cluster | | | | | | | |
| GO:0038162 | erythropoietin- | 0.006667992 | 2 | 2 | 0.16 | 2.18E+00 | BP | UP |
| | mediated signaling | | | | | | | |
| | pathway | | | | | | | |
| GO:0046502 | uroporphyrinogen | 0.006667992 | 2 | 2 | 0.16 | 2.18E+00 | BP | UP |
| | III metabolic | | | | | | | |
| | process | | | | | | | |
| GO:0050747 | positive regulation | 0.006667992 | 2 | 2 | 0.16 | 2.18E+00 | BP | UP |
| | of lipoprotein | | | | | | | |
| | metabolic process | | | | | | | |
| GO:0071284 | cellular response | 0.006667992 | 2 | 2 | 0.16 | 2.18E+00 | BP | UP |
| | to lead ion | | | | | | | |
| GO:1902855 | regulation of | 0.006667992 | 2 | 2 | 0.16 | 2.18E+00 | BP | UP |
| | nonmotile primary | | | | | | | |
| | cilium assembly | | | | | | | |
| GO:0042073 | intraciliary | 0.006874301 | 37 | 14 | 3.02 | 2.16E+00 | BP | UP |
| | transport | | | | | | | |
| GO:0050771 | negative regulation | 0.007449195 | 36 | 8 | 2.94 | 2.13E+00 | BP | UP |
| | of axonogenesis | | | | | | | |
| GO:0001501 | skeletal system | 0.008300412 | 454 | 52 | 37.08 | 2.08E+00 | BP | UP |
| | development | | | | | | | |
| GO:0032482 | Rab protein | 0.008636413 | 528 | 59 | 43.13 | 2.06E+00 | BP | UP |
| | signal transduction | | | | | | | |
| GO:0006691 | leukotriene | 0.008839224 | 23 | 6 | 1.88 | 2.05E+00 | BP | UP |
| | metabolic process | | | | | | | |
| GO:0035162 | embryonic | 0.008839224 | 23 | 6 | 1.88 | 2.05E+00 | BP | UP |
| | hemopoiesis | | | | | | | |
| GO:0042373 | vitamin K | 0.009011774 | 6 | 3 | 0.49 | 2.05E+00 | BP | UP |
| | metabolic process | | | | | | | |
| GO:0045213 | neurotransmitter | 0.009011774 | 6 | 3 | 0.49 | 2.05E+00 | BP | UP |
| | receptor metabolic | | | | | | | |
| | process | | | | | | | |
| GO:0018342 | protein prenylation | 0.009171769 | 11 | 4 | 0.9 | 2.04E+00 | BP | UP |
| GO:0042178 | xenobiotic | 0.009171769 | 11 | 4 | 0.9 | 2.04E+00 | BP | UP |
| | catabolic process | | | | | | | |
| GO:0071243 | cellular response to | 0.009171769 | 11 | 4 | 0.9 | 2.04E+00 | BP | UP |
| | arsenic-containing | | | | | | | |
| | substance | | | | | | | |

TABLE 64
GO Biological Pathway Terms by P Value for Transcripts Decreased by HDAC6 Inhibitor Treatment

| | Anno- | Signif- | | | Neg Log | | GO | |
|---|---|---|---|---|---|---|---|---|
| GO Term | tated | icant | Expected | p-value | (p-value) | Term/Pathway | type | Response |
| GO:0042832 | 24 | 12 | 1.91 | 6.86E−08 | 7.16E+00 | defense response | BP | DOWN |
| | | | | | | to protozoan | | |
| GO:0050853 | 38 | 17 | 3.03 | 7.40E−08 | 7.13E+00 | B cell receptor | BP | DOWN |
| | | | | | | signaling pathway | | |
| GO:0045087 | 471 | 99 | 37.52 | 9.05E−08 | 7.04E+00 | innate immune | BP | DOWN |
| | | | | | | response | | |
| GO:0006954 | 502 | 100 | 39.99 | 1.18E−07 | 6.93E+00 | inflammatory | BP | DOWN |
| | | | | | | response | | |
| GO:0071222 | 142 | 35 | 11.31 | 3.88E−07 | 6.41E+00 | cellular response to | BP | DOWN |
| | | | | | | lipopolysaccharide | | |
| GO:0051301 | 568 | 86 | 45.24 | 5.65E−07 | 6.25E+00 | cell division | BP | DOWN |
| GO:0043123 | 159 | 32 | 12.66 | 8.91E−07 | 6.05E+00 | positive regulation | BP | DOWN |
| | | | | | | of I-kappaB kinase/ | | |
| | | | | | | NF-kappaB signaling | | |
| GO:0034976 | 128 | 40 | 10.2 | 1.09E−06 | 5.96E+00 | response to | BP | DOWN |
| | | | | | | endoplasmic | | |
| | | | | | | reticulum stress | | |
| GO:0030968 | 51 | 16 | 4.06 | 1.20E−06 | 5.92E+00 | endoplasmic reticulum | BP | DOWN |
| | | | | | | unfolded protein | | |
| | | | | | | response | | |
| GO:0043547 | 424 | 62 | 33.77 | 2.29E−06 | 5.64E+00 | positive regulation | BP | DOWN |
| | | | | | | of GTPase activity | | |
| GO:0051092 | 100 | 23 | 7.97 | 2.85E−06 | 5.55E+00 | positive regulation | BP | DOWN |
| | | | | | | of NF-kappaB | | |
| | | | | | | transcription factor | | |
| | | | | | | activity | | |
| GO:0006909 | 139 | 34 | 11.07 | 4.54E−06 | 5.34E+00 | phagocytosis | BP | DOWN |
| GO:0008360 | 119 | 25 | 9.48 | 6.11E−06 | 5.21E+00 | regulation of | BP | DOWN |
| | | | | | | cell shape | | |
| GO:0006886 | 780 | 115 | 62.13 | 6.62E−06 | 5.18E+00 | intracellular | BP | DOWN |
| | | | | | | protein transport | | |
| GO:0061028 | 29 | 11 | 2.31 | 7.12E−06 | 5.15E+00 | establishment of | BP | DOWN |
| | | | | | | endothelial barrier | | |
| GO:0032496 | 287 | 63 | 22.86 | 7.31E−06 | 5.14E+00 | response to | BP | DOWN |
| | | | | | | lipopolysaccharide | | |
| GO:0050830 | 58 | 16 | 4.62 | 7.81E−06 | 5.11E+00 | defense response | BP | DOWN |
| | | | | | | to Gram-positive | | |
| | | | | | | bacterium | | |
| GO:0007067 | 358 | 59 | 28.52 | 9.11E−06 | 5.04E+00 | mitotic nuclear | BP | DOWN |
| | | | | | | division | | |
| GO:0007568 | 261 | 42 | 20.79 | 9.39E−06 | 5.03E+00 | aging | BP | DOWN |
| GO:0035335 | 93 | 21 | 7.41 | 1.03E−05 | 4.99E+00 | peptidyl-tyrosine | BP | DOWN |
| | | | | | | dephosphorylation | | |
| GO:0006898 | 203 | 35 | 16.17 | 1.12E−05 | 4.95E+00 | receptor-mediated | BP | DOWN |
| | | | | | | endocytosis | | |
| GO:0002755 | 16 | 8 | 1.27 | 1.15E−05 | 4.94E+00 | MyD88-dependent | BP | DOWN |
| | | | | | | toll-like receptor | | |
| | | | | | | signaling pathway | | |
| GO:0007179 | 135 | 29 | 10.75 | 2.01E−05 | 4.70E+00 | transforming growth | BP | DOWN |
| | | | | | | factor beta receptor | | |
| | | | | | | signaling pathway | | |
| GO:0071260 | 69 | 17 | 5.5 | 2.11E−05 | 4.67E+00 | cellular response | BP | DOWN |
| | | | | | | to mechanical | | |
| | | | | | | stimulus | | |
| GO:0018279 | 13 | 7 | 1.04 | 2.24E−05 | 4.65E+00 | protein N-linked | BP | DOWN |
| | | | | | | glycosylation via | | |
| | | | | | | asparagine | | |
| GO:0034097 | 575 | 89 | 45.8 | 2.54E−05 | 4.60E+00 | response to cytokine | BP | DOWN |
| GO:0006888 | 70 | 17 | 5.58 | 2.59E−05 | 4.59E+00 | ER to Golgi | BP | DOWN |
| | | | | | | vesicle-mediated | | |
| | | | | | | transport | | |
| GO:0032733 | 23 | 9 | 1.83 | 3.69E−05 | 4.43E+00 | positive regulation | BP | DOWN |
| | | | | | | of interleukin-10 | | |
| | | | | | | production | | |
| GO:0030433 | 46 | 13 | 3.66 | 4.16E−05 | 4.38E+00 | ER-associated | BP | DOWN |
| | | | | | | ubiquitin-dependent | | |
| | | | | | | protein catabolic | | |
| | | | | | | process | | |
| GO:0048008 | 46 | 13 | 3.66 | 4.16E−05 | 4.38E+00 | platelet-derived | BP | DOWN |
| | | | | | | growth factor | | |
| | | | | | | receptor signaling | | |
| | | | | | | pathway | | |
| GO:0032695 | 14 | 7 | 1.12 | 4.18E−05 | 4.38E+00 | negative regulation | BP | DOWN |
| | | | | | | of interleukin-12 | | |
| | | | | | | production | | |
| GO:0035556 | 2309 | 318 | 183.92 | 5.35E−05 | 4.27E+00 | intracellular | BP | DOWN |
| | | | | | | signal transduction | | |
| GO:0002532 | 35 | 11 | 2.79 | 5.50E−05 | 4.26E+00 | production of | BP | DOWN |
| | | | | | | molecular mediator | | |
| | | | | | | involved in | | |
| | | | | | | inflammatory | | |
| | | | | | | response | | |
| GO:0043407 | 62 | 20 | 4.94 | 6.99E−05 | 4.16E+00 | negative regulation | BP | DOWN |
| | | | | | | of MAP kinase | | |
| | | | | | | activity | | |
| GO:0034122 | 22 | 10 | 1.75 | 8.33E−05 | 4.08E+00 | negative regulation | BP | DOWN |
| | | | | | | of toll-like receptor | | |
| | | | | | | signaling pathway | | |
| GO:0000188 | 20 | 8 | 1.59 | 8.40E−05 | 4.08E+00 | inactivation of | BP | DOWN |
| | | | | | | MAPK activity | | |
| GO:0030890 | 37 | 11 | 2.95 | 9.71E−05 | 4.01E+00 | positive regulation | BP | DOWN |
| | | | | | | of B cell | | |
| | | | | | | proliferation | | |
| GO:0051241 | 900 | 125 | 71.69 | 0.000101 | 4.00E+00 | NA | BP | DOWN |
| GO:0009967 | 1128 | 163 | 89.85 | 0.0001069 | 3.97E+00 | positive regulation | BP | DOWN |
| | | | | | | of signal transduction | | |
| GO:0032956 | 253 | 38 | 20.15 | 0.0001123 | 3.95E+00 | regulation of actin | BP | DOWN |
| | | | | | | cytoskeleton | | |
| | | | | | | organization | | |
| GO:0008285 | 546 | 79 | 43.49 | 0.0001132 | 3.95E+00 | negative regulation | BP | DOWN |
| | | | | | | of cell proliferation | | |
| GO:0051607 | 174 | 29 | 13.86 | 0.0001144 | 3.94E+00 | defense response to | BP | DOWN |
| | | | | | | virus | | |
| GO:0043406 | 169 | 32 | 13.46 | 0.0001159 | 3.94E+00 | positive regulation | BP | DOWN |
| | | | | | | of MAP kinase | | |
| | | | | | | activity | | |
| GO:0001525 | 388 | 60 | 30.91 | 0.0001204 | 3.92E+00 | angiogenesis | BP | DOWN |
| GO:0050728 | 92 | 22 | 7.33 | 0.0001407 | 3.85E+00 | negative regulation | BP | DOWN |
| | | | | | | of inflammatory | | |
| | | | | | | response | | |
| GO:0000186 | 45 | 12 | 3.58 | 0.000151 | 3.82E+00 | activation of | BP | DOWN |
| | | | | | | MAPKK activity | | |
| GO:0045078 | 12 | 6 | 0.96 | 0.0001536 | 3.81E+00 | positive regulation | BP | DOWN |
| | | | | | | of interferon-gamma | | |
| | | | | | | biosynthetic process | | |
| GO:0050777 | 103 | 20 | 8.2 | 0.0001593 | 3.80E+00 | negative regulation | BP | DOWN |
| | | | | | | of immune response | | |
| GO:0045944 | 952 | 107 | 75.83 | 0.0001688 | 3.77E+00 | positive regulation | BP | DOWN |
| | | | | | | of transcription | | |
| | | | | | | from RNA polymerase | | |
| | | | | | | II promoter | | |
| GO:0002677 | 5 | 4 | 0.4 | 0.0001878 | 3.73E+00 | negative regulation | BP | DOWN |
| | | | | | | of chronic inflammatory | | |
| | | | | | | response | | |
| GO:0050732 | 36 | 13 | 2.87 | 0.0001879 | 3.73E+00 | negative regulation | BP | DOWN |
| | | | | | | of peptidyl-tyrosine | | |
| | | | | | | phosphorylation | | |
| GO:0030889 | 17 | 7 | 1.35 | 0.0001912 | 3.72E+00 | negative regulation | BP | DOWN |
| | | | | | | of B cell proliferation | | |
| GO:0006919 | 74 | 16 | 5.89 | 0.0001947 | 3.71E+00 | activation of cysteine- | BP | DOWN |
| | | | | | | type endopeptidase | | |
| | | | | | | activity involved in | | |
| | | | | | | apoptotic process | | |
| GO:0046777 | 209 | 32 | 16.65 | 0.0002649 | 3.58E+00 | protein | BP | DOWN |
| | | | | | | autophosphorylation | | |
| GO:0016446 | 13 | 6 | 1.04 | 0.000266 | 3.58E+00 | somatic | BP | DOWN |
| | | | | | | hypermutation of | | |
| | | | | | | immunoglobulin | | |
| | | | | | | genes | | |
| GO:0001782 | 33 | 12 | 2.63 | 0.0002882 | 3.54E+00 | B cell homeostasis | BP | DOWN |
| GO:0032727 | 13 | 8 | 1.04 | 0.0003034 | 3.52E+00 | positive regulation | BP | DOWN |
| | | | | | | of interferon-alpha | | |
| | | | | | | production | | |
| GO:0002634 | 9 | 5 | 0.72 | 0.0003057 | 3.51E+00 | regulation of | BP | DOWN |
| | | | | | | germinal center | | |
| | | | | | | formation | | |
| GO:1901701 | 735 | 122 | 58.54 | 0.0003302 | 3.48E+00 | NA | BP | DOWN |
| GO:0010506 | 230 | 34 | 18.32 | 0.0003395 | 3.47E+00 | regulation of | BP | DOWN |
| | | | | | | autophagy | | |
| GO:0032088 | 63 | 14 | 5.02 | 0.0003562 | 3.45E+00 | negative regulation | BP | DOWN |
| | | | | | | of NF-kappaB | | |
| | | | | | | transcription | | |
| | | | | | | factor activity | | |
| GO:0007229 | 84 | 19 | 6.69 | 0.0003616 | 3.44E+00 | integrin-mediated | BP | DOWN |
| | | | | | | signaling pathway | | |
| GO:2000377 | 134 | 23 | 10.67 | 0.0003627 | 3.44E+00 | regulation of | BP | DOWN |
| | | | | | | reactive oxygen | | |
| | | | | | | species metabolic | | |
| | | | | | | process | | |
| GO:0001783 | 24 | 8 | 1.91 | 0.0003675 | 3.43E+00 | B cell apoptotic | BP | DOWN |
| | | | | | | process | | |
| GO:0007030 | 79 | 16 | 6.29 | 0.0004276 | 3.37E+00 | Golgi organization | BP | DOWN |
| GO:0010212 | 140 | 27 | 11.15 | 0.0004326 | 3.36E+00 | response to | BP | DOWN |
| | | | | | | ionizing radiation | | |
| GO:0046325 | 14 | 6 | 1.12 | 0.0004341 | 3.36E+00 | negative regulation | BP | DOWN |
| | | | | | | of glucose import | | |
| GO:0031396 | 155 | 30 | 12.35 | 0.0004538 | 3.34E+00 | regulation of | BP | DOWN |
| | | | | | | protein | | |
| | | | | | | ubiquitination | | |
| GO:0045071 | 37 | 10 | 2.95 | 0.0004728 | 3.33E+00 | negative regulation | BP | DOWN |
| | | | | | | of viral genome | | |
| | | | | | | replication | | |
| GO:0001788 | 3 | 3 | 0.24 | 0.0005045 | 3.30E+00 | antibody-dependent | BP | DOWN |
| | | | | | | cellular cytotoxicity | | |
| GO:0010835 | 3 | 3 | 0.24 | 0.0005045 | 3.30E+00 | regulation of | BP | DOWN |
| | | | | | | protein ADP- | | |
| | | | | | | ribosylation | | |
| GO:0051088 | 3 | 3 | 0.24 | 0.0005045 | 3.30E+00 | PMA-inducible | BP | DOWN |
| | | | | | | membrane protein | | |
| | | | | | | ectodomain | | |
| | | | | | | proteolysis | | |
| GO:0002467 | 15 | 9 | 1.19 | 0.0005225 | 3.28E+00 | germinal center | BP | DOWN |
| | | | | | | formation | | |
| GO:0001798 | 6 | 4 | 0.48 | 0.0005278 | 3.28E+00 | positive regulation | BP | DOWN |
| | | | | | | of type IIa | | |
| | | | | | | hypersensitivity | | |
| GO:0060696 | 6 | 4 | 0.48 | 0.0005278 | 3.28E+00 | regulation of | BP | DOWN |
| | | | | | | phospholipid | | |
| | | | | | | catabolic process | | |
| GO:0016064 | 97 | 25 | 7.73 | 0.0005592 | 3.25E+00 | immunoglobulin | BP | DOWN |
| | | | | | | mediated immune | | |
| | | | | | | response | | |
| GO:0006977 | 10 | 5 | 0.8 | 0.0005713 | 3.24E+00 | DNA damage | BP | DOWN |
| | | | | | | response, signal | | |
| | | | | | | transduction by p53 | | |
| | | | | | | class mediator | | |
| | | | | | | resulting in cell | | |
| | | | | | | cycle arrest | | |
| GO:0033623 | 10 | 5 | 0.8 | 0.0005713 | 3.24E+00 | regulation of | BP | DOWN |
| | | | | | | integrin activation | | |
| GO:0048012 | 10 | 5 | 0.8 | 0.0005713 | 3.24E+00 | hepatocyte growth | BP | DOWN |
| | | | | | | factor receptor | | |
| | | | | | | signaling pathway | | |
| GO:0006302 | 147 | 24 | 11.71 | 0.0005824 | 3.23E+00 | double-strand break | BP | DOWN |
| | | | | | | repair | | |
| GO:0045766 | 105 | 19 | 8.36 | 0.0005834 | 3.23E+00 | positive regulation | BP | DOWN |
| | | | | | | of angiogenesis | | |
| GO:0042093 | 38 | 10 | 3.03 | 0.0005963 | 3.22E+00 | T-helper cell | BP | DOWN |
| | | | | | | differentiation | | |
| GO:0006950 | 2860 | 427 | 227.81 | 0.0006061 | 3.22E+00 | response to stress | BP | DOWN |
| GO:0010832 | 15 | 6 | 1.19 | 0.0006749 | 3.17E+00 | negative regulation | BP | DOWN |
| | | | | | | of myotube | | |
| | | | | | | differentiation | | |
| GO:0032703 | 15 | 6 | 1.19 | 0.0006749 | 3.17E+00 | negative regulation | BP | DOWN |
| | | | | | | of interleukin-2 | | |
| | | | | | | production | | |
| GO:0000281 | 26 | 8 | 2.07 | 0.0006762 | 3.17E+00 | mitotic cytokinesis | BP | DOWN |
| GO:0002639 | 33 | 9 | 2.63 | 0.0008402 | 3.08E+00 | positive regulation | BP | DOWN |
| | | | | | | of immunoglobulin | | |
| | | | | | | production | | |
| GO:0032715 | 33 | 9 | 2.63 | 0.0008402 | 3.08E+00 | negative regulation | BP | DOWN |
| | | | | | | of interleukin-6 | | |
| | | | | | | production | | |
| GO:0007184 | 21 | 7 | 1.67 | 0.0008608 | 3.07E+00 | SMAD protein | BP | DOWN |
| | | | | | | import into nucleus | | |
| GO:0050871 | 64 | 19 | 5.1 | 0.0008659 | 3.06E+00 | positive regulation | BP | DOWN |
| | | | | | | of B cell activation | | |
| GO:0032735 | 27 | 8 | 2.15 | 0.0008944 | 3.05E+00 | positive regulation | BP | DOWN |
| | | | | | | of interleukin-12 | | |
| | | | | | | production | | |
| GO:0050856 | 27 | 8 | 2.15 | 0.0008944 | 3.05E+00 | regulation of T | BP | DOWN |
| | | | | | | cell receptor | | |
| | | | | | | signaling pathway | | |
| GO:0043542 | 134 | 22 | 10.67 | 0.0008955 | 3.05E+00 | endothelial cell | BP | DOWN |
| | | | | | | migration | | |
| GO:0010770 | 143 | 23 | 11.39 | 0.0009264 | 3.03E+00 | positive regulation | BP | DOWN |
| | | | | | | of cell morphogenesis | | |
| | | | | | | involved in | | |
| | | | | | | differentiation | | |
| GO:0016310 | 1852 | 270 | 147.52 | 0.0009287 | 3.03E+00 | phosphorylation | BP | DOWN |
| GO:0002456 | 97 | 22 | 7.73 | 0.0009601 | 3.02E+00 | T cell mediated | BP | DOWN |
| | | | | | | immunity | | |
| GO:0031571 | 21 | 10 | 1.67 | 0.0009674 | 3.01E+00 | mitotic G1 DNA | BP | DOWN |
| | | | | | | damage checkpoint | | |
| GO:0002313 | 11 | 5 | 0.88 | 0.0009788 | 3.01E+00 | mature B cell | BP | DOWN |
| | | | | | | differentiation | | |
| | | | | | | involved in immune | | |
| | | | | | | response | | |
| GO:0006614 | 11 | 5 | 0.88 | 0.0009788 | 3.01E+00 | SRP-dependent | BP | DOWN |
| | | | | | | cotranslational | | |
| | | | | | | protein targeting | | |
| | | | | | | to membrane | | |
| GO:0007035 | 11 | 5 | 0.88 | 0.0009788 | 3.01E+00 | vacuolar acidification | BP | DOWN |
| GO:0007076 | 11 | 5 | 0.88 | 0.0009788 | 3.01E+00 | mitotic chromosome | BP | DOWN |
| | | | | | | condensation | | |
| GO:0060670 | 11 | 5 | 0.88 | 0.0009788 | 3.01E+00 | branching involved | BP | DOWN |
| | | | | | | in labyrinthine | | |
| | | | | | | layer morphogenesis | | |
| GO:0048569 | 16 | 6 | 1.27 | 0.0010074 | 3.00E+00 | post-embryonic | BP | DOWN |
| | | | | | | organ development | | |
| GO:0044728 | 62 | 13 | 4.94 | 0.0010248 | 2.99E+00 | NA | BP | DOWN |
| GO:0070301 | 62 | 13 | 4.94 | 0.0010248 | 2.99E+00 | cellular response | BP | DOWN |
| | | | | | | to hydrogen peroxide | | |
| GO:0046649 | 542 | 105 | 43.17 | 0.001026 | 2.99E+00 | lymphocyte activation | BP | DOWN |
| GO:0050663 | 134 | 26 | 10.67 | 0.0011219 | 2.95E+00 | cytokine secretion | BP | DOWN |
| GO:0043124 | 48 | 11 | 3.82 | 0.0011385 | 2.94E+00 | negative regulation | BP | DOWN |
| | | | | | | of I-kappaB kinase/ | | |
| | | | | | | NF-kappaB signaling | | |
| GO:0050766 | 48 | 11 | 3.82 | 0.0011385 | 2.94E+00 | positive regulation | BP | DOWN |
| | | | | | | of phagocytosis | | |
| GO:0031936 | 7 | 4 | 0.56 | 0.0011536 | 2.94E+00 | negative regulation | BP | DOWN |
| | | | | | | of chromatin silencing | | |
| GO:0034162 | 7 | 4 | 0.56 | 0.0011536 | 2.94E+00 | toll-like receptor | BP | DOWN |
| | | | | | | 9 signaling pathway | | |
| GO:0045348 | 7 | 4 | 0.56 | 0.0011536 | 2.94E+00 | positive regulation | BP | DOWN |
| | | | | | | of MHC class II | | |
| | | | | | | biosynthetic process | | |
| GO:0045359 | 7 | 4 | 0.56 | 0.0011536 | 2.94E+00 | positive regulation | BP | DOWN |
| | | | | | | of interferon-beta | | |
| | | | | | | biosynthetic process | | |
| GO:0045714 | 7 | 4 | 0.56 | 0.0011536 | 2.94E+00 | regulation of low- | BP | DOWN |
| | | | | | | density lipoprotein | | |
| | | | | | | particle receptor | | |
| | | | | | | biosynthetic process | | |
| GO:0033003 | 35 | 12 | 2.79 | 0.0011597 | 2.94E+00 | regulation of mast | BP | DOWN |
| | | | | | | cell activation | | |
| GO:0030334 | 561 | 88 | 44.69 | 0.00118 | 2.93E+00 | regulation of cell | BP | DOWN |
| | | | | | | migration | | |
| GO:0051347 | 421 | 85 | 33.53 | 0.001265 | 2.90E+00 | positive regulation | BP | DOWN |
| | | | | | | of transferase activity | | |
| GO:0071897 | 86 | 20 | 6.85 | 0.0013238 | 2.88E+00 | DNA biosynthetic | BP | DOWN |
| | | | | | | process | | |
| GO:0033628 | 35 | 9 | 2.79 | 0.0013308 | 2.88E+00 | regulation of cell | BP | DOWN |
| | | | | | | adhesion mediated | | |
| | | | | | | by integrin | | |
| GO:1902580 | 925 | 116 | 73.68 | 0.0013729 | 2.86E+00 | NA | BP | DOWN |
| GO:0002708 | 105 | 25 | 8.36 | 0.0013871 | 2.86E+00 | NA | BP | DOWN |
| GO:0006281 | 388 | 57 | 30.91 | 0.0013897 | 2.86E+00 | DNA repair | BP | DOWN |
| GO:0051983 | 64 | 13 | 5.1 | 0.0013964 | 2.85E+00 | regulation of | BP | DOWN |
| | | | | | | chromosome | | |
| | | | | | | segregation | | |
| GO:0010564 | 414 | 62 | 32.98 | 0.0014128 | 2.85E+00 | regulation of cell | BP | DOWN |
| | | | | | | cycle process | | |
| GO:0051591 | 96 | 17 | 7.65 | 0.0014216 | 2.85E+00 | response to cAMP | BP | DOWN |
| GO:0045910 | 17 | 6 | 1.35 | 0.0014527 | 2.84E+00 | negative regulation | BP | DOWN |
| | | | | | | of DNA | | |
| | | | | | | recombination | | |
| GO:0060251 | 17 | 6 | 1.35 | 0.0014527 | 2.84E+00 | NA | BP | DOWN |
| GO:1902236 | 17 | 6 | 1.35 | 0.0014527 | 2.84E+00 | negative regulation | BP | DOWN |
| | | | | | | of endoplasmic | | |
| | | | | | | reticulum stress- | | |
| | | | | | | induced intrinsic | | |
| | | | | | | apoptotic signaling | | |
| | | | | | | pathway | | |
| GO:0031331 | 186 | 32 | 14.82 | 0.0015012 | 2.82E+00 | NA | BP | DOWN |
| GO:0001774 | 12 | 5 | 0.96 | 0.0015682 | 2.80E+00 | microglial cell | BP | DOWN |
| | | | | | | activation | | |
| GO:0006266 | 12 | 5 | 0.96 | 0.0015682 | 2.80E+00 | DNA ligation | BP | DOWN |
| GO:0061299 | 12 | 5 | 0.96 | 0.0015682 | 2.80E+00 | retina vasculature | BP | DOWN |
| | | | | | | morphogenesis in | | |
| | | | | | | camera-type eye | | |
| GO:0010543 | 23 | 7 | 1.83 | 0.0015758 | 2.80E+00 | regulation of | BP | DOWN |
| | | | | | | platelet activation | | |
| GO:0030335 | 322 | 47 | 25.65 | 0.0016097 | 2.79E+00 | positive regulation | BP | DOWN |
| | | | | | | of cell migration | | |
| GO:0006611 | 50 | 11 | 3.98 | 0.0016249 | 2.79E+00 | protein export from | BP | DOWN |
| | | | | | | nucleus | | |
| GO:0048013 | 36 | 9 | 2.87 | 0.0016506 | 2.78E+00 | ephrin receptor | BP | DOWN |
| | | | | | | signaling pathway | | |
| GO:0065008 | 2841 | 321 | 226.29 | 0.001663 | 2.78E+00 | NA | BP | DOWN |
| GO:0034113 | 43 | 10 | 3.43 | 0.0016783 | 2.78E+00 | heterotypic cell- | BP | DOWN |
| | | | | | | cell adhesion | | |
| GO:0030198 | 195 | 28 | 15.53 | 0.0016955 | 2.77E+00 | extracellular | BP | DOWN |
| | | | | | | matrix organization | | |
| GO:0032269 | 747 | 112 | 59.5 | 0.0017104 | 2.77E+00 | negative regulation | BP | DOWN |
| | | | | | | of cellular protein | | |
| | | | | | | metabolic process | | |
| GO:0006260 | 239 | 38 | 19.04 | 0.0017124 | 2.77E+00 | DNA replication | BP | DOWN |
| GO:0050727 | 216 | 47 | 17.2 | 0.0017363 | 2.76E+00 | regulation of | BP | DOWN |
| | | | | | | inflammatory response | | |
| GO:0001932 | 939 | 160 | 74.79 | 0.001775 | 2.75E+00 | regulation of protein | BP | DOWN |
| | | | | | | phosphorylation | | |
| GO:0016197 | 196 | 28 | 15.61 | 0.0018314 | 2.74E+00 | endosomal transport | BP | DOWN |
| GO:0051640 | 365 | 49 | 29.07 | 0.0018538 | 2.73E+00 | organelle localization | BP | DOWN |
| GO:0032648 | 37 | 12 | 2.95 | 0.0018772 | 2.73E+00 | regulation of | BP | DOWN |
| | | | | | | interferon-beta | | |
| | | | | | | production | | |
| GO:0001922 | 4 | 3 | 0.32 | 0.0018977 | 2.72E+00 | B-1 B cell | BP | DOWN |
| | | | | | | homeostasis | | |
| GO:0010694 | 4 | 3 | 0.32 | 0.0018977 | 2.72E+00 | positive regulation | BP | DOWN |
| | | | | | | of alkaline | | |
| | | | | | | phosphatase activity | | |
| GO:0034154 | 4 | 3 | 0.32 | 0.0018977 | 2.72E+00 | toll-like receptor | BP | DOWN |
| | | | | | | 7 signaling pathway | | |
| GO:0038094 | 4 | 3 | 0.32 | 0.0018977 | 2.72E+00 | Fc-gamma receptor | BP | DOWN |
| | | | | | | signaling pathway | | |
| GO:0038145 | 4 | 3 | 0.32 | 0.0018977 | 2.72E+00 | macrophage colony- | BP | DOWN |
| | | | | | | stimulating factor | | |
| | | | | | | signaling pathway | | |
| GO:0045356 | 4 | 3 | 0.32 | 0.0018977 | 2.72E+00 | positive regulation | BP | DOWN |
| | | | | | | of interferon-alpha | | |
| | | | | | | biosynthetic process | | |
| GO:0045719 | 4 | 3 | 0.32 | 0.0018977 | 2.72E+00 | negative regulation | BP | DOWN |
| | | | | | | of glycogen | | |
| | | | | | | biosynthetic process | | |
| GO:1900225 | 4 | 3 | 0.32 | 0.0018977 | 2.72E+00 | regulation of NLRP3 | BP | DOWN |
| | | | | | | inflammasome | | |
| | | | | | | complex assembly | | |
| GO:2000617 | 4 | 3 | 0.32 | 0.0018977 | 2.72E+00 | positive regulation | BP | DOWN |
| | | | | | | of histone H3-K9 | | |
| | | | | | | acetylation | | |
| GO:0007155 | 1252 | 171 | 99.72 | 0.0019005 | 2.72E+00 | cell adhesion | BP | DOWN |
| GO:0046627 | 30 | 8 | 2.39 | 0.0019024 | 2.72E+00 | negative regulation | BP | DOWN |
| | | | | | | of insulin receptor | | |
| | | | | | | signaling pathway | | |
| GO:0060706 | 30 | 8 | 2.39 | 0.0019024 | 2.72E+00 | cell differentiation | BP | DOWN |
| | | | | | | involved in | | |
| | | | | | | embryonic placenta | | |
| | | | | | | development | | |
| GO:0051129 | 501 | 66 | 39.91 | 0.001973 | 2.70E+00 | NA | BP | DOWN |
| GO:0008347 | 37 | 9 | 2.95 | 0.0020292 | 2.69E+00 | glial cell migration | BP | DOWN |
| GO:0032757 | 37 | 9 | 2.95 | 0.0020292 | 2.69E+00 | positive regulation | BP | DOWN |
| | | | | | | of interleukin-8 | | |
| | | | | | | production | | |
| GO:2000278 | 37 | 9 | 2.95 | 0.0020292 | 2.69E+00 | regulation of DNA | BP | DOWN |
| | | | | | | biosynthetic process | | |
| GO:0034248 | 18 | 6 | 1.43 | 0.0020333 | 2.69E+00 | NA | BP | DOWN |
| GO:0071407 | 353 | 44 | 28.12 | 0.0020511 | 2.69E+00 | cellular response | BP | DOWN |
| | | | | | | to organic cyclic | | |
| | | | | | | compound | | |
| GO:0050731 | 145 | 26 | 11.55 | 0.0020543 | 2.69E+00 | positive regulation | BP | DOWN |
| | | | | | | of peptidyl-tyrosine | | |
| | | | | | | phosphorylation | | |
| GO:0002828 | 24 | 7 | 1.91 | 0.0020733 | 2.68E+00 | regulation of type | BP | DOWN |
| | | | | | | 2 immune response | | |
| GO:0030866 | 24 | 7 | 1.91 | 0.0020733 | 2.68E+00 | cortical actin | BP | DOWN |
| | | | | | | cytoskeleton | | |
| | | | | | | organization | | |
| GO:0060445 | 24 | 7 | 1.91 | 0.0020733 | 2.68E+00 | branching involved | BP | DOWN |
| | | | | | | in salivary gland | | |
| | | | | | | morphogenesis | | |
| GO:0031349 | 209 | 49 | 16.65 | 0.0021491 | 2.67E+00 | NA | BP | DOWN |
| GO:0034138 | 8 | 4 | 0.64 | 0.0021618 | 2.67E+00 | toll-like receptor | BP | DOWN |
| | | | | | | 3 signaling pathway | | |
| GO:0043373 | 8 | 4 | 0.64 | 0.0021618 | 2.67E+00 | NA | BP | DOWN |
| GO:0048102 | 8 | 4 | 0.64 | 0.0021618 | 2.67E+00 | autophagic cell | BP | DOWN |
| | | | | | | death | | |
| GO:2000059 | 8 | 4 | 0.64 | 0.0021618 | 2.67E+00 | negative regulation of | BP | DOWN |
| | | | | | | protein ubiquitination | | |
| | | | | | | involved in ubiquitin- | | |
| | | | | | | dependent protein | | |
| | | | | | | catabolic process | | |
| GO:0032874 | 120 | 23 | 9.56 | 0.0022793 | 2.64E+00 | positive regulation | BP | DOWN |
| | | | | | | of stress-activated | | |
| | | | | | | MAPK cascade | | |
| GO:0042542 | 107 | 23 | 8.52 | 0.002328 | 2.63E+00 | response to | BP | DOWN |
| | | | | | | hydrogen peroxide | | |
| GO:0001820 | 13 | 5 | 1.04 | 0.0023822 | 2.62E+00 | serotonin secretion | BP | DOWN |
| GO:0043306 | 13 | 5 | 1.04 | 0.0023822 | 2.62E+00 | positive regulation | BP | DOWN |
| | | | | | | of mast cell | | |
| | | | | | | degranulation | | |
| GO:0051336 | 1087 | 146 | 86.58 | 0.0024335 | 2.61E+00 | NA | BP | DOWN |
| GO:0070507 | 118 | 19 | 9.4 | 0.0024436 | 2.61E+00 | regulation of | BP | DOWN |
| | | | | | | microtubule | | |
| | | | | | | cytoskeleton | | |
| | | | | | | organization | | |
| GO:0048754 | 172 | 25 | 13.7 | 0.0024635 | 2.61E+00 | branching | BP | DOWN |
| | | | | | | morphogenesis of an | | |
| | | | | | | epithelial tube | | |
| GO:0060603 | 38 | 9 | 3.03 | 0.0024739 | 2.61E+00 | mammary gland duct | BP | DOWN |
| | | | | | | morphogenesis | | |
| GO:0018107 | 68 | 13 | 5.42 | 0.0024775 | 2.61E+00 | peptidyl-threonine | BP | DOWN |
| | | | | | | phosphorylation | | |
| GO:0033157 | 297 | 38 | 23.66 | 0.0025334 | 2.60E+00 | regulation of | BP | DOWN |
| | | | | | | intracellular | | |
| | | | | | | protein transport | | |
| GO:0009968 | 943 | 135 | 75.11 | 0.0026041 | 2.58E+00 | negative regulation | BP | DOWN |
| | | | | | | of signal transduction | | |
| GO:0006897 | 495 | 91 | 39.43 | 0.0026762 | 2.57E+00 | endocytosis | BP | DOWN |
| GO:0002548 | 25 | 7 | 1.99 | 0.002684 | 2.57E+00 | monocyte | BP | DOWN |
| | | | | | | chemotaxis | | |
| GO:0045577 | 25 | 7 | 1.99 | 0.002684 | 2.57E+00 | regulation of B | BP | DOWN |
| | | | | | | cell differentiation | | |
| GO:0045672 | 25 | 7 | 1.99 | 0.002684 | 2.57E+00 | positive regulation | BP | DOWN |
| | | | | | | of osteoclast | | |
| | | | | | | differentiation | | |
| GO:0042176 | 326 | 54 | 25.97 | 0.0027702 | 2.56E+00 | regulation of | BP | DOWN |
| | | | | | | protein catabolic | | |
| | | | | | | process | | |
| GO:0044130 | 19 | 6 | 1.51 | 0.0027735 | 2.56E+00 | negative regulation | BP | DOWN |
| | | | | | | of growth of | | |
| | | | | | | symbiont in host | | |
| GO:0061099 | 19 | 6 | 1.51 | 0.0027735 | 2.56E+00 | negative regulation | BP | DOWN |
| | | | | | | of protein tyrosine | | |
| | | | | | | kinase activity | | |
| GO:0030330 | 56 | 15 | 4.46 | 0.0028153 | 2.55E+00 | DNA damage | BP | DOWN |
| | | | | | | response, signal | | |
| | | | | | | transduction by p53 | | |
| | | | | | | class mediator | | |
| GO:0033036 | 2273 | 288 | 181.05 | 0.0028921 | 2.54E+00 | NA | BP | DOWN |
| GO:0070059 | 56 | 15 | 4.46 | 0.0029358 | 2.53E+00 | intrinsic apoptotic | BP | DOWN |
| | | | | | | signaling pathway | | |
| | | | | | | in response to | | |
| | | | | | | endoplasmic | | |
| | | | | | | reticulum stress | | |
| GO:0008284 | 779 | 91 | 62.05 | 0.0029703 | 2.53E+00 | positive regulation | BP | DOWN |
| | | | | | | of cell proliferation | | |
| GO:0032722 | 39 | 9 | 3.11 | 0.0029925 | 2.52E+00 | positive regulation | BP | DOWN |
| | | | | | | of chemokine | | |
| | | | | | | production | | |
| GO:0050868 | 81 | 18 | 6.45 | 0.0030352 | 2.52E+00 | negative regulation | BP | DOWN |
| | | | | | | of T cell activation | | |
| GO:0046822 | 184 | 26 | 14.66 | 0.0030384 | 2.52E+00 | regulation of | BP | DOWN |
| | | | | | | nucleocytoplasmic | | |
| | | | | | | transport | | |
| GO:0030100 | 180 | 33 | 14.34 | 0.0030981 | 2.51E+00 | regulation of | BP | DOWN |
| | | | | | | endocytosis | | |
| GO:0002474 | 54 | 11 | 4.3 | 0.0031054 | 2.51E+00 | antigen processing | BP | DOWN |
| | | | | | | and presentation of | | |
| | | | | | | peptide antigen via | | |
| | | | | | | MHC class I | | |
| GO:0045454 | 62 | 12 | 4.94 | 0.0032167 | 2.49E+00 | cell redox | BP | DOWN |
| | | | | | | homeostasis | | |
| GO:0071375 | 206 | 33 | 16.41 | 0.003219 | 2.49E+00 | cellular response | BP | DOWN |
| | | | | | | to peptide hormone | | |
| | | | | | | stimulus | | |
| GO:0002690 | 70 | 13 | 5.58 | 0.0032324 | 2.49E+00 | positive regulation | BP | DOWN |
| | | | | | | of leukocyte | | |
| | | | | | | chemotaxis | | |
| GO:0042267 | 44 | 12 | 3.5 | 0.0034221 | 2.47E+00 | natural killer cell | BP | DOWN |
| | | | | | | mediated cytotoxicity | | |
| GO:0033198 | 26 | 7 | 2.07 | 0.0034238 | 2.47E+00 | response to ATP | BP | DOWN |
| GO:0002685 | 127 | 26 | 10.12 | 0.0034544 | 2.46E+00 | regulation of | BP | DOWN |
| | | | | | | leukocyte migration | | |
| GO:0034123 | 14 | 5 | 1.12 | 0.0034644 | 2.46E+00 | positive regulation | BP | DOWN |
| | | | | | | of toll-like | | |
| | | | | | | receptor signaling | | |
| | | | | | | pathway | | |
| GO:0070234 | 14 | 5 | 1.12 | 0.0034644 | 2.46E+00 | positive regulation | BP | DOWN |
| | | | | | | of T cell apoptotic | | |
| | | | | | | process | | |
| GO:0071800 | 14 | 5 | 1.12 | 0.0034644 | 2.46E+00 | podosome assembly | BP | DOWN |
| GO:0042771 | 40 | 9 | 3.19 | 0.0035932 | 2.44E+00 | intrinsic apoptotic | BP | DOWN |
| | | | | | | signaling pathway | | |
| | | | | | | in response to DNA | | |
| | | | | | | damage by p53 class | | |
| | | | | | | mediator | | |
| GO:0044765 | 3216 | 381 | 256.16 | 0.0035989 | 2.44E+00 | NA | BP | DOWN |
| GO:0032436 | 55 | 11 | 4.38 | 0.0036079 | 2.44E+00 | positive regulation | BP | DOWN |
| | | | | | | of proteasomal | | |
| | | | | | | ubiquitin-dependent | | |
| | | | | | | protein catabolic | | |
| | | | | | | process | | |
| GO:0032760 | 55 | 11 | 4.38 | 0.0036079 | 2.44E+00 | positive regulation | BP | DOWN |
| | | | | | | of tumor necrosis | | |
| | | | | | | factor production | | |
| GO:0032147 | 201 | 38 | 16.01 | 0.0036176 | 2.44E+00 | activation of | BP | DOWN |
| | | | | | | protein kinase | | |
| | | | | | | activity | | |
| GO:0010470 | 33 | 8 | 2.63 | 0.0036441 | 2.44E+00 | regulation of | BP | DOWN |
| | | | | | | gastrulation | | |
| GO:0050829 | 33 | 8 | 2.63 | 0.0036441 | 2.44E+00 | defense response to | BP | DOWN |
| | | | | | | Gram-negative | | |
| | | | | | | bacterium | | |
| GO:0002517 | 9 | 4 | 0.72 | 0.0036467 | 2.44E+00 | T cell tolerance | BP | DOWN |
| | | | | | | induction | | |
| GO:0002645 | 9 | 4 | 0.72 | 0.0036467 | 2.44E+00 | positive regulation | BP | DOWN |
| | | | | | | of tolerance | | |
| | | | | | | induction | | |
| GO:0000086 | 71 | 13 | 5.66 | 0.0036746 | 2.43E+00 | G2/M transition of | BP | DOWN |
| | | | | | | mitotic cell cycle | | |
| GO:0010629 | 1271 | 142 | 101.24 | 0.0036854 | 2.43E+00 | negative regulation | BP | DOWN |
| | | | | | | of gene expression | | |
| GO:0030593 | 63 | 12 | 5.02 | 0.0036915 | 2.43E+00 | neutrophil | BP | DOWN |
| | | | | | | chemotaxis | | |
| GO:0035994 | 20 | 6 | 1.59 | 0.0036981 | 2.43E+00 | response to muscle | BP | DOWN |
| | | | | | | stretch | | |
| GO:0051043 | 20 | 6 | 1.59 | 0.0036981 | 2.43E+00 | NA | BP | DOWN |
| GO:0071822 | 1352 | 167 | 107.69 | 0.0037502 | 2.43E+00 | NA | BP | DOWN |
| GO:0007010 | 967 | 132 | 77.02 | 0.0037941 | 2.42E+00 | cytoskeleton | BP | DOWN |
| | | | | | | organization | | |
| GO:0043065 | 488 | 69 | 38.87 | 0.0037994 | 2.42E+00 | positive regulation | BP | DOWN |
| | | | | | | of apoptotic process | | |
| GO:0031334 | 159 | 23 | 12.66 | 0.0038085 | 2.42E+00 | positive regulation | BP | DOWN |
| | | | | | | of protein complex | | |
| | | | | | | assembly | | |
| GO:0050680 | 114 | 18 | 9.08 | 0.0038856 | 2.41E+00 | negative regulation | BP | DOWN |
| | | | | | | of epithelial cell | | |
| | | | | | | proliferation | | |
| GO:0007584 | 178 | 25 | 14.18 | 0.0039123 | 2.41E+00 | response to nutrient | BP | DOWN |
| GO:0043524 | 141 | 21 | 11.23 | 0.0039212 | 2.41E+00 | negative regulation | BP | DOWN |
| | | | | | | of neuron apoptotic | | |
| | | | | | | process | | |
| GO:0070374 | 123 | 19 | 9.8 | 0.0039324 | 2.41E+00 | positive regulation | BP | DOWN |
| | | | | | | of ERK1 and ERK2 | | |
| | | | | | | cascade | | |
| GO:0071363 | 410 | 72 | 32.66 | 0.00398 | 2.40E+00 | cellular response | BP | DOWN |
| | | | | | | to growth factor | | |
| | | | | | | stimulus | | |
| GO:0022411 | 366 | 44 | 29.15 | 0.0040537 | 2.39E+00 | NA | BP | DOWN |
| GO:0009267 | 160 | 23 | 12.74 | 0.0041212 | 2.38E+00 | cellular response | BP | DOWN |
| | | | | | | to starvation | | |
| GO:0034644 | 56 | 11 | 4.46 | 0.0041733 | 2.38E+00 | cellular response | BP | DOWN |
| | | | | | | to UV | | |
| GO:2001236 | 157 | 26 | 12.51 | 0.0041837 | 2.38E+00 | regulation of | BP | DOWN |
| | | | | | | extrinsic apoptotic | | |
| | | | | | | signaling pathway | | |
| GO:0045581 | 27 | 7 | 2.15 | 0.0043091 | 2.37E+00 | negative regulation | BP | DOWN |
| | | | | | | of T cell | | |
| | | | | | | differentiation | | |
| GO:0034502 | 34 | 8 | 2.71 | 0.0044383 | 2.35E+00 | protein localization | BP | DOWN |
| | | | | | | to chromosome | | |
| GO:0035924 | 34 | 8 | 2.71 | 0.0044383 | 2.35E+00 | cellular response | BP | DOWN |
| | | | | | | to vascular | | |
| | | | | | | endothelial growth | | |
| | | | | | | factor stimulus | | |
| GO:0038061 | 34 | 8 | 2.71 | 0.0044383 | 2.35E+00 | NIK/NF-kappaB | BP | DOWN |
| | | | | | | signaling | | |
| GO:0001812 | 5 | 3 | 0.4 | 0.0044625 | 2.35E+00 | positive regulation | BP | DOWN |
| | | | | | | of type I | | |
| | | | | | | hypersensitivity | | |
| GO:0002513 | 5 | 3 | 0.4 | 0.0044625 | 2.35E+00 | tolerance induction | BP | DOWN |
| | | | | | | to self antigen | | |
| GO:0022614 | 5 | 3 | 0.4 | 0.0044625 | 2.35E+00 | membrane to | BP | DOWN |
| | | | | | | membrane docking | | |
| GO:0023035 | 5 | 3 | 0.4 | 0.0044625 | 2.35E+00 | CD40 signaling | BP | DOWN |
| | | | | | | pathway | | |
| GO:0032211 | 5 | 3 | 0.4 | 0.0044625 | 2.35E+00 | negative regulation | BP | DOWN |
| | | | | | | of telomere | | |
| | | | | | | maintenance via | | |
| | | | | | | telomerase | | |
| GO:0034638 | 5 | 3 | 0.4 | 0.0044625 | 2.35E+00 | phosphatidylcholine | BP | DOWN |
| | | | | | | catabolic process | | |
| GO:0045345 | 5 | 3 | 0.4 | 0.0044625 | 2.35E+00 | positive regulation | BP | DOWN |
| | | | | | | of MHC class I | | |
| | | | | | | biosynthetic process | | |
| GO:0050859 | 5 | 3 | 0.4 | 0.0044625 | 2.35E+00 | negative regulation | BP | DOWN |
| | | | | | | of B cell receptor | | |
| | | | | | | signaling pathway | | |
| GO:0051256 | 5 | 3 | 0.4 | 0.0044625 | 2.35E+00 | mitotic spindle | BP | DOWN |
| | | | | | | midzone assembly | | |
| GO:0060058 | 5 | 3 | 0.4 | 0.0044625 | 2.35E+00 | positive regulation | BP | DOWN |
| | | | | | | of apoptotic process | | |
| | | | | | | involved in mammary | | |
| | | | | | | gland involution | | |
| GO:1901026 | 5 | 3 | 0.4 | 0.0044625 | 2.35E+00 | ripoptosome assembly | BP | DOWN |
| | | | | | | involved in | | |
| | | | | | | necroptotic process | | |
| GO:1902563 | 5 | 3 | 0.4 | 0.0044625 | 2.35E+00 | NA | BP | DOWN |
| GO:0010811 | 98 | 16 | 7.81 | 0.0044991 | 2.35E+00 | positive regulation | BP | DOWN |
| | | | | | | of cell-substrate | | |
| | | | | | | adhesion | | |
| GO:0007169 | 457 | 74 | 36.4 | 0.0045191 | 2.34E+00 | transmembrane | BP | DOWN |
| | | | | | | receptor protein | | |
| | | | | | | tyrosine kinase | | |
| | | | | | | signaling pathway | | |
| GO:0002824 | 107 | 24 | 8.52 | 0.0045953 | 2.34E+00 | NA | BP | DOWN |
| GO:0070372 | 188 | 31 | 14.97 | 0.0045983 | 2.34E+00 | regulation of ERK1 | BP | DOWN |
| | | | | | | and ERK2 cascade | | |
| GO:0045860 | 356 | 72 | 28.36 | 0.004648 | 2.33E+00 | positive regulation | BP | DOWN |
| | | | | | | of protein kinase | | |
| | | | | | | activity | | |
| GO:0006801 | 49 | 10 | 3.9 | 0.0046509 | 2.33E+00 | superoxide | BP | DOWN |
| | | | | | | metabolic process | | |
| GO:0007041 | 49 | 10 | 3.9 | 0.0046509 | 2.33E+00 | lysosomal transport | BP | DOWN |
| GO:0070527 | 49 | 10 | 3.9 | 0.0046509 | 2.33E+00 | platelet aggregation | BP | DOWN |
| GO:0019221 | 289 | 42 | 23.02 | 0.0047076 | 2.33E+00 | cytokine-mediated | BP | DOWN |
| | | | | | | signaling pathway | | |
| GO:0032091 | 65 | 12 | 5.18 | 0.00481 | 2.32E+00 | negative regulation | BP | DOWN |
| | | | | | | of protein binding | | |
| GO:0007160 | 162 | 23 | 12.9 | 0.0048113 | 2.32E+00 | cell-matrix adhesion | BP | DOWN |
| GO:0042098 | 162 | 23 | 12.9 | 0.0048113 | 2.32E+00 | T cell | BP | DOWN |
| | | | | | | proliferation | | |
| GO:0030099 | 322 | 52 | 25.65 | 0.0048154 | 2.32E+00 | myeloid cell | BP | DOWN |
| | | | | | | differentiation | | |
| GO:0048011 | 21 | 6 | 1.67 | 0.0048331 | 2.32E+00 | neurotrophin TRK | BP | DOWN |
| | | | | | | receptor signaling | | |
| | | | | | | pathway | | |
| GO:0030220 | 15 | 5 | 1.19 | 0.0048591 | 2.31E+00 | platelet formation | BP | DOWN |
| GO:0045780 | 15 | 5 | 1.19 | 0.0048591 | 2.31E+00 | positive regulation | BP | DOWN |
| | | | | | | of bone resorption | | |
| GO:0045892 | 1041 | 111 | 82.92 | 0.0048796 | 2.31E+00 | negative regulation | BP | DOWN |
| | | | | | | of transcription, | | |
| | | | | | | DNA-templated | | |
| GO:0042326 | 361 | 68 | 28.75 | 0.0048885 | 2.31E+00 | negative regulation | BP | DOWN |
| | | | | | | of phosphorylation | | |
| GO:0046637 | 42 | 9 | 3.35 | 0.005075 | 2.29E+00 | NA | BP | DOWN |
| GO:0032868 | 203 | 32 | 16.17 | 0.0051959 | 2.28E+00 | response to insulin | BP | DOWN |
| GO:0010976 | 182 | 25 | 14.5 | 0.0052307 | 2.28E+00 | positive regulation | BP | DOWN |
| | | | | | | of neuron projection | | |
| | | | | | | development | | |
| GO:0015031 | 1439 | 192 | 114.62 | 0.0052701 | 2.28E+00 | protein transport | BP | DOWN |
| GO:0046677 | 51 | 13 | 4.06 | 0.0052865 | 2.28E+00 | response to | BP | DOWN |
| | | | | | | antibiotic | | |
| GO:0006875 | 374 | 46 | 29.79 | 0.0053312 | 2.27E+00 | cellular metal ion | BP | DOWN |
| | | | | | | homeostasis | | |
| GO:0071417 | 371 | 56 | 29.55 | 0.0053356 | 2.27E+00 | cellular response | BP | DOWN |
| | | | | | | to organonitrogen | | |
| | | | | | | compound | | |
| GO:0007257 | 28 | 7 | 2.23 | 0.0053569 | 2.27E+00 | activation of JUN | BP | DOWN |
| | | | | | | kinase activity | | |
| GO:0032459 | 28 | 7 | 2.23 | 0.0053569 | 2.27E+00 | regulation of | BP | DOWN |
| | | | | | | protein oligomerization | | |
| GO:0061001 | 28 | 7 | 2.23 | 0.0053569 | 2.27E+00 | regulation of | BP | DOWN |
| | | | | | | dendritic spine | | |
| | | | | | | morphogenesis | | |
| GO:0098751 | 28 | 7 | 2.23 | 0.0053569 | 2.27E+00 | NA | BP | DOWN |
| GO:0009888 | 1742 | 204 | 138.75 | 0.005358 | 2.27E+00 | tissue development | BP | DOWN |
| GO:0046834 | 35 | 8 | 2.79 | 0.0053591 | 2.27E+00 | lipid phosphorylation | BP | DOWN |
| GO:0046635 | 50 | 10 | 3.98 | 0.0054074 | 2.27E+00 | positive regulation | BP | DOWN |
| | | | | | | of alpha-beta T | | |
| | | | | | | cell activation | | |
| GO:0072503 | 313 | 40 | 24.93 | 0.0054274 | 2.27E+00 | NA | BP | DOWN |
| GO:0050864 | 102 | 33 | 8.12 | 0.0054306 | 2.27E+00 | regulation of B | BP | DOWN |
| | | | | | | cell activation | | |
| GO:0006906 | 100 | 16 | 7.97 | 0.0055006 | 2.26E+00 | vesicle fusion | BP | DOWN |
| GO:0007098 | 58 | 11 | 4.62 | 0.0055146 | 2.26E+00 | centrosome cycle | BP | DOWN |
| GO:0045931 | 109 | 17 | 8.68 | 0.0055945 | 2.25E+00 | positive regulation | BP | DOWN |
| | | | | | | of mitotic cell | | |
| | | | | | | cycle | | |
| GO:0002726 | 10 | 4 | 0.8 | 0.005697 | 2.24E+00 | positive regulation | BP | DOWN |
| | | | | | | of T cell cytokine | | |
| | | | | | | production | | |
| GO:0006268 | 10 | 4 | 0.8 | 0.005697 | 2.24E+00 | DNA unwinding | BP | DOWN |
| | | | | | | involved in DNA | | |
| | | | | | | replication | | |
| GO:0006465 | 10 | 4 | 0.8 | 0.005697 | 2.24E+00 | signal peptide | BP | DOWN |
| | | | | | | processing | | |
| GO:0019471 | 10 | 4 | 0.8 | 0.005697 | 2.24E+00 | 4-hydroxyproline | BP | DOWN |
| | | | | | | metabolic process | | |
| GO:0032740 | 10 | 4 | 0.8 | 0.005697 | 2.24E+00 | positive regulation | BP | DOWN |
| | | | | | | of interleukin-17 | | |
| | | | | | | production | | |
| GO:0036010 | 10 | 4 | 0.8 | 0.005697 | 2.24E+00 | protein localization | BP | DOWN |
| | | | | | | to endosome | | |
| GO:0060768 | 10 | 4 | 0.8 | 0.005697 | 2.24E+00 | regulation of | BP | DOWN |
| | | | | | | epithelial cell | | |
| | | | | | | proliferation | | |
| | | | | | | involved in prostate | | |
| | | | | | | gland development | | |
| GO:0048522 | 4067 | 503 | 323.95 | 0.0057003 | 2.24E+00 | NA | BP | DOWN |
| GO:0010038 | 272 | 34 | 21.67 | 0.005923 | 2.23E+00 | response to metal | BP | DOWN |
| | | | | | | ion | | |
| GO:0006955 | 988 | 195 | 78.7 | 0.0059234 | 2.23E+00 | immune response | BP | DOWN |
| GO:0031663 | 45 | 11 | 3.58 | 0.005931 | 2.23E+00 | lipopolysaccharide- | BP | DOWN |
| | | | | | | mediated signaling | | |
| | | | | | | pathway | | |
| GO:0007595 | 43 | 9 | 3.43 | 0.0059741 | 2.22E+00 | lactation | BP | DOWN |
| GO:0097305 | 348 | 47 | 27.72 | 0.0060002 | 2.22E+00 | response to alcohol | BP | DOWN |
| GO:0002312 | 57 | 17 | 4.54 | 0.0060551 | 2.22E+00 | B cell activation | BP | DOWN |
| | | | | | | involved in immune | | |
| | | | | | | response | | |
| GO:0007249 | 224 | 47 | 17.84 | 0.0060668 | 2.22E+00 | I-kappaB kinase/NF- | BP | DOWN |
| | | | | | | kappaB signaling | | |
| GO:0034142 | 22 | 6 | 1.75 | 0.0062043 | 2.21E+00 | toll-like receptor | BP | DOWN |
| | | | | | | 4 signaling pathway | | |
| GO:0044843 | 147 | 32 | 11.71 | 0.006218 | 2.21E+00 | NA | BP | DOWN |
| GO:0042981 | 1264 | 165 | 100.68 | 0.0062422 | 2.20E+00 | regulation of | BP | DOWN |
| | | | | | | apoptotic process | | |
| GO:0051272 | 338 | 52 | 26.92 | 0.0062483 | 2.20E+00 | positive regulation | BP | DOWN |
| | | | | | | of cellular component | | |
| | | | | | | movement | | |
| GO:0007043 | 59 | 11 | 4.7 | 0.0063018 | 2.20E+00 | cell-cell junction | BP | DOWN |
| | | | | | | assembly | | |
| GO:0014065 | 84 | 14 | 6.69 | 0.0063107 | 2.20E+00 | phosphatidylinositol | BP | DOWN |
| | | | | | | 3-kinase signaling | | |
| GO:0001970 | 2 | 2 | 0.16 | 0.006341 | 2.20E+00 | positive regulation | BP | DOWN |
| | | | | | | of activation of | | |
| | | | | | | membrane attack | | |
| | | | | | | complex | | |
| GO:0002760 | 2 | 2 | 0.16 | 0.006341 | 2.20E+00 | positive regulation | BP | DOWN |
| | | | | | | of antimicrobial | | |
| | | | | | | humoral response | | |
| GO:0031938 | 2 | 2 | 0.16 | 0.006341 | 2.20E+00 | regulation of | BP | DOWN |
| | | | | | | chromatin silencing | | |
| | | | | | | at telomere | | |
| GO:0032661 | 2 | 2 | 0.16 | 0.006341 | 2.20E+00 | NA | BP | DOWN |
| GO:0032804 | 2 | 2 | 0.16 | 0.006341 | 2.20E+00 | negative regulation | BP | DOWN |
| | | | | | | of low-density | | |
| | | | | | | lipoprotein particle | | |
| | | | | | | receptor catabolic | | |
| | | | | | | process | | |
| GO:0033277 | 2 | 2 | 0.16 | 0.006341 | 2.20E+00 | abortive mitotic cell | BP | DOWN |
| | | | | | | cycle | | |
| GO:0034136 | 2 | 2 | 0.16 | 0.006341 | 2.20E+00 | negative regulation | BP | DOWN |
| | | | | | | of toll-like receptor | | |
| | | | | | | 2 signaling pathway | | |
| GO:0035026 | 2 | 2 | 0.16 | 0.006341 | 2.20E+00 | leading edge cell | BP | DOWN |
| | | | | | | differentiation | | |
| GO:0043316 | 2 | 2 | 0.16 | 0.006341 | 2.20E+00 | cytotoxic T cell | BP | DOWN |
| | | | | | | degranulation | | |
| GO:0043686 | 2 | 2 | 0.16 | 0.006341 | 2.20E+00 | co-translational | BP | DOWN |
| | | | | | | protein modification | | |
| GO:0044338 | 2 | 2 | 0.16 | 0.006341 | 2.20E+00 | canonical Wnt | BP | DOWN |
| | | | | | | signaling pathway | | |
| | | | | | | involved in | | |
| | | | | | | mesenchymal stem | | |
| | | | | | | cell differentiation | | |
| GO:0044339 | 2 | 2 | 0.16 | 0.006341 | 2.20E+00 | canonical Wnt | BP | DOWN |
| | | | | | | signaling pathway | | |
| | | | | | | involved in osteoblast | | |
| | | | | | | differentiation | | |
| GO:0045368 | 2 | 2 | 0.16 | 0.006341 | 2.20E+00 | positive regulation | BP | DOWN |
| | | | | | | of interleukin-13 | | |
| | | | | | | biosynthetic process | | |
| GO:0045401 | 2 | 2 | 0.16 | 0.006341 | 2.20E+00 | positive regulation | BP | DOWN |
| | | | | | | of interleukin-3 | | |
| | | | | | | biosynthetic process | | |
| GO:0045425 | 2 | 2 | 0.16 | 0.006341 | 2.20E+00 | positive regulation | BP | DOWN |
| | | | | | | of granulocyte | | |
| | | | | | | macrophage colony- | | |
| | | | | | | stimulating factor | | |
| | | | | | | biosynthetic process | | |
| GO:0046110 | 2 | 2 | 0.16 | 0.006341 | 2.20E+00 | NA | BP | DOWN |
| GO:0051563 | 2 | 2 | 0.16 | 0.006341 | 2.20E+00 | smooth endoplasmic | BP | DOWN |
| | | | | | | reticulum calcium | | |
| | | | | | | ion homeostasis | | |
| GO:0060101 | 2 | 2 | 0.16 | 0.006341 | 2.20E+00 | negative regulation | BP | DOWN |
| | | | | | | of phagocytosis, | | |
| | | | | | | engulfment | | |
| GO:0071226 | 2 | 2 | 0.16 | 0.006341 | 2.20E+00 | cellular response to | BP | DOWN |
| | | | | | | molecule of fungal | | |
| | | | | | | origin | | |
| GO:0072573 | 2 | 2 | 0.16 | 0.006341 | 2.20E+00 | tolerance induction | BP | DOWN |
| | | | | | | to lipopolysaccharide | | |
| GO:0072719 | 2 | 2 | 0.16 | 0.006341 | 2.20E+00 | cellular response to | BP | DOWN |
| | | | | | | cisplatin | | |
| GO:1900248 | 2 | 2 | 0.16 | 0.006341 | 2.20E+00 | negative regulation | BP | DOWN |
| | | | | | | of cytoplasmic | | |
| | | | | | | translational | | |
| | | | | | | elongation | | |
| GO:1902525 | 2 | 2 | 0.16 | 0.006341 | 2.20E+00 | regulation of protein | BP | DOWN |
| | | | | | | monoubiquitination | | |
| GO:2000417 | 2 | 2 | 0.16 | 0.006341 | 2.20E+00 | negative regulation | BP | DOWN |
| | | | | | | of eosinophil | | |
| | | | | | | migration | | |
| GO:0002446 | 27 | 9 | 2.15 | 0.0065569 | 2.18E+00 | neutrophil mediated | BP | DOWN |
| | | | | | | immunity | | |
| GO:0010165 | 29 | 7 | 2.31 | 0.0065846 | 2.18E+00 | response to X-ray | BP | DOWN |
| GO:0033598 | 29 | 7 | 2.31 | 0.0065846 | 2.18E+00 | mammary gland | BP | DOWN |
| | | | | | | epithelial cell | | |
| | | | | | | proliferation | | |
| GO:0034698 | 29 | 7 | 2.31 | 0.0065846 | 2.18E+00 | response to | BP | DOWN |
| | | | | | | gonadotropin | | |
| GO:0045736 | 29 | 7 | 2.31 | 0.0065846 | 2.18E+00 | negative regulation | BP | DOWN |
| | | | | | | of cyclin-dependent | | |
| | | | | | | protein serine/ | | |
| | | | | | | threonine kinase | | |
| | | | | | | activity | | |
| GO:1903035 | 136 | 31 | 10.83 | 0.0065993 | 2.18E+00 | NA | BP | DOWN |
| GO:0034349 | 16 | 5 | 1.27 | 0.0066099 | 2.18E+00 | glial cell apoptotic | BP | DOWN |
| | | | | | | process | | |
| GO:0071236 | 16 | 5 | 1.27 | 0.0066099 | 2.18E+00 | cellular response to | BP | DOWN |
| | | | | | | antibiotic | | |
| GO:0051054 | 129 | 19 | 10.28 | 0.0066389 | 2.18E+00 | positive regulation | BP | DOWN |
| | | | | | | of DNA metabolic | | |
| | | | | | | process | | |
| GO:0002429 | 114 | 33 | 9.08 | 0.006679 | 2.18E+00 | immune response- | BP | DOWN |
| | | | | | | activating cell | | |
| | | | | | | surface receptor | | |
| | | | | | | signaling pathway | | |
| GO:0061572 | 111 | 17 | 8.84 | 0.0067256 | 2.17E+00 | actin filament bundle | BP | DOWN |
| | | | | | | organization | | |
| GO:0032649 | 80 | 18 | 6.37 | 0.0068137 | 2.17E+00 | regulation of | BP | DOWN |
| | | | | | | interferon-gamma | | |
| | | | | | | production | | |
| GO:2000106 | 82 | 17 | 6.53 | 0.0068511 | 2.16E+00 | NA | BP | DOWN |
| GO:0051239 | 2276 | 304 | 181.29 | 0.0068606 | 2.16E+00 | NA | BP | DOWN |
| GO:0001568 | 539 | 81 | 42.93 | 0.0070841 | 2.15E+00 | blood vessel | BP | DOWN |
| | | | | | | development | | |
| GO:0001816 | 521 | 100 | 41.5 | 0.0070958 | 2.15E+00 | cytokine production | BP | DOWN |
| GO:0006974 | 616 | 96 | 49.07 | 0.0071558 | 2.15E+00 | cellular response to | BP | DOWN |
| | | | | | | DNA damage stimulus | | |
| GO:0010498 | 336 | 52 | 26.76 | 0.0073831 | 2.13E+00 | proteasomal protein | BP | DOWN |
| | | | | | | catabolic process | | |
| GO:0051302 | 236 | 30 | 18.8 | 0.0073911 | 2.13E+00 | regulation of cell | BP | DOWN |
| | | | | | | division | | |
| GO:0050715 | 77 | 13 | 6.13 | 0.007461 | 2.13E+00 | positive regulation | BP | DOWN |
| | | | | | | of cytokine secretion | | |
| GO:0002697 | 274 | 57 | 21.82 | 0.0074629 | 2.13E+00 | NA | BP | DOWN |
| GO:0055074 | 309 | 39 | 24.61 | 0.0074716 | 2.13E+00 | calcium ion | BP | DOWN |
| | | | | | | homeostasis | | |
| GO:0051235 | 280 | 38 | 22.3 | 0.007573 | 2.12E+00 | NA | BP | DOWN |
| GO:0050710 | 37 | 8 | 2.95 | 0.0076295 | 2.12E+00 | negative regulation | BP | DOWN |
| | | | | | | of cytokine secretion | | |
| GO:0061098 | 37 | 8 | 2.95 | 0.0076295 | 2.12E+00 | positive regulation | BP | DOWN |
| | | | | | | of protein tyrosine | | |
| | | | | | | kinase activity | | |
| GO:0042325 | 1182 | 191 | 94.15 | 0.0077278 | 2.11E+00 | regulation of | BP | DOWN |
| | | | | | | phosphorylation | | |
| GO:0043112 | 130 | 23 | 10.35 | 0.0077488 | 2.11E+00 | receptor metabolic | BP | DOWN |
| | | | | | | process | | |
| GO:0008608 | 23 | 6 | 1.83 | 0.007838 | 2.11E+00 | attachment of spindle | BP | DOWN |
| | | | | | | microtubules to | | |
| | | | | | | kinetochore | | |
| GO:0030511 | 23 | 6 | 1.83 | 0.007838 | 2.11E+00 | positive regulation | BP | DOWN |
| | | | | | | of transforming | | |
| | | | | | | growth factor beta | | |
| | | | | | | receptor signaling | | |
| | | | | | | pathway | | |
| GO:0030279 | 69 | 12 | 5.5 | 0.0078499 | 2.11E+00 | negative regulation | BP | DOWN |
| | | | | | | of ossification | | |
| GO:00512601 | 247 | 31 | 19.67 | 0.0078519 | 2.11E+00 | protein | BP | DOWN |
| | | | | | | homooligomerization | | |
| GO:0035850 | 30 | 7 | 2.39 | 0.0080096 | 2.10E+00 | NA | BP | DOWN |
| GO:0042269 | 30 | 7 | 2.39 | 0.0080096 | 2.10E+00 | regulation of natural | BP | DOWN |
| | | | | | | killer cell mediated | | |
| | | | | | | cytotoxicity | | |
| GO:0033043 | 987 | 133 | 78.62 | 0.0081055 | 2.09E+00 | regulation of | BP | DOWN |
| | | | | | | organelle organization | | |
| GO:0043086 | 687 | 95 | 54.72 | 0.0082488 | 2.08E+00 | negative regulation | BP | DOWN |
| | | | | | | of catalytic activity | | |
| GO:0061515 | 53 | 10 | 4.22 | 0.0082634 | 2.08E+00 | NA | BP | DOWN |
| GO:0002366 | 187 | 44 | 14.9 | 0.0082848 | 2.08E+00 | leukocyte activation | BP | DOWN |
| | | | | | | involved in immune | | |
| | | | | | | response | | |
| GO:0045351 | 13 | 7 | 1.04 | 0.0083479 | 2.08E+00 | type I interferon | BP | DOWN |
| | | | | | | biosynthetic process | | |
| GO:2000257 | 13 | 6 | 1.04 | 0.00836 | 2.08E+00 | NA | BP | DOWN |
| GO:0032494 | 11 | 4 | 0.88 | 0.0083931 | 2.08E+00 | response to | BP | DOWN |
| | | | | | | peptidoglycan | | |
| GO:0035589 | 11 | 4 | 0.88 | 0.0083931 | 2.08E+00 | G-protein coupled | BP | DOWN |
| | | | | | | purinergic nucleotide | | |
| | | | | | | receptor signaling | | |
| | | | | | | pathway | | |
| GO:0043312 | 11 | 4 | 0.88 | 0.0083931 | 2.08E+00 | neutrophil | BP | DOWN |
| | | | | | | degranulation | | |
| GO:0045651 | 11 | 4 | 0.88 | 0.0083931 | 2.08E+00 | positive regulation | BP | DOWN |
| | | | | | | of macrophage | | |
| | | | | | | differentiation | | |
| GO:0002756 | 6 | 3 | 0.48 | 0.0083971 | 2.08E+00 | MyD88-independent | BP | DOWN |
| | | | | | | toll-like receptor | | |
| | | | | | | signaling pathway | | |
| GO:0014010 | 6 | 3 | 0.48 | 0.0083971 | 2.08E+00 | Schwann cell | BP | DOWN |
| | | | | | | proliferation | | |
| GO:0022417 | 6 | 3 | 0.48 | 0.0083971 | 2.08E+00 | protein maturation | BP | DOWN |
| | | | | | | by protein folding | | |
| GO:0051133 | 6 | 3 | 0.48 | 0.0083971 | 2.08E+00 | NA | BP | DOWN |
| GO:0090073 | 6 | 3 | 0.48 | 0.0083971 | 2.08E+00 | positive regulation | BP | DOWN |
| | | | | | | of protein | | |
| | | | | | | homodimerization | | |
| | | | | | | activity | | |
| GO:2001046 | 6 | 3 | 0.48 | 0.0083971 | 2.08E+00 | positive regulation | BP | DOWN |
| | | | | | | of integrin-mediated | | |
| | | | | | | signaling pathway | | |
| GO:0072001 | 289 | 39 | 23.02 | 0.0084278 | 2.07E+00 | renal system | BP | DOWN |
| | | | | | | development | | |
| GO:0032944 | 178 | 36 | 14.18 | 0.0086435 | 2.06E+00 | NA | BP | DOWN |
| GO:1902582 | 1132 | 155 | 90.17 | 0.0087243 | 2.06E+00 | NA | BP | DOWN |
| GO:0050482 | 17 | 5 | 1.35 | 0.0087587 | 2.06E+00 | arachidonic acid | BP | DOWN |
| | | | | | | secretion | | |
| GO:0060337 | 17 | 5 | 1.35 | 0.0087587 | 2.06E+00 | type I interferon | BP | DOWN |
| | | | | | | signaling pathway | | |
| GO:0070570 | 17 | 5 | 1.35 | 0.0087587 | 2.06E+00 | regulation of neuron | BP | DOWN |
| | | | | | | projection | | |
| | | | | | | regeneration | | |
| GO:0060759 | 70 | 12 | 5.58 | 0.0088052 | 2.06E+00 | NA | BP | DOWN |
| GO:0071456 | 105 | 16 | 8.36 | 0.0088089 | 2.06E+00 | cellular response to | BP | DOWN |
| | | | | | | hypoxia | | |
| GO:0009892 | 2268 | 280 | 180.65 | 0.0089297 | 2.05E+00 | negative regulation | BP | DOWN |
| | | | | | | of metabolic process | | |
| GO:0002704 | 38 | 8 | 3.03 | 0.0090043 | 2.05E+00 | NA | BP | DOWN |
| GO:0043551 | 38 | 8 | 3.03 | 0.0090043 | 2.05E+00 | regulation of | BP | DOWN |
| | | | | | | phosphatidylinositol | | |
| | | | | | | 3-kinase activity | | |
| GO:0035303 | 137 | 22 | 10.91 | 0.0090199 | 2.04E+00 | regulation of | BP | DOWN |
| | | | | | | dephosphorylation | | |
| GO:0034341 | 62 | 11 | 4.94 | 0.0091999 | 2.04E+00 | response to | BP | DOWN |
| | | | | | | interferon-gamma | | |
| GO:0042991 | 79 | 13 | 6.29 | 0.0092494 | 2.03E+00 | transcription factor | BP | DOWN |
| | | | | | | import into nucleus | | |
| GO:0042110 | 383 | 65 | 30.51 | 0.0092674 | 2.03E+00 | T cell activation | BP | DOWN |
| GO:0045088 | 175 | 43 | 13.94 | 0.0093654 | 2.03E+00 | regulation of innate | BP | DOWN |
| | | | | | | immune response | | |
| GO:0036473 | 54 | 10 | 4.3 | 0.0094357 | 2.03E+00 | NA | BP | DOWN |
| GO:1901215 | 165 | 27 | 13.14 | 0.0094868 | 2.02E+00 | negative regulation | BP | DOWN |
| | | | | | | of neuron death | | |
| GO:2000134 | 45 | 16 | 3.58 | 0.0095282 | 2.02E+00 | negative regulation | BP | DOWN |
| | | | | | | of G1/S transition of | | |
| | | | | | | mitotic cell cycle | | |
| GO:0034329 | 130 | 23 | 10.35 | 0.0095674 | 2.02E+00 | cell junction | BP | DOWN |
| | | | | | | assembly | | |
| GO:0051093 | 865 | 97 | 68.9 | 0.0095849 | 2.02E+00 | NA | BP | DOWN |
| GO:0051247 | 1086 | 170 | 86.5 | 0.009637 | 2.02E+00 | positive regulation | BP | DOWN |
| | | | | | | of protein metabolic | | |
| | | | | | | process | | |
| GO:0015804 | 31 | 7 | 2.47 | 0.0096494 | 2.02E+00 | neutral amino acid | BP | DOWN |
| | | | | | | transport | | |
| GO:0051091 | 189 | 37 | 15.05 | 0.0097129 | 2.01E+00 | positive regulation | BP | DOWN |
| | | | | | | of sequence-specific | | |
| | | | | | | DNA binding | | |
| | | | | | | transcription factor | | |
| | | | | | | activity | | |
| GO:0030336 | 174 | 25 | 13.86 | 0.0097308 | 2.01E+00 | negative regulation | BP | DOWN |
| | | | | | | of cell migration | | |
| GO:0045191 | 24 | 6 | 1.91 | 0.0097595 | 2.01E+00 | regulation of isotype | BP | DOWN |
| | | | | | | switching | | |
| GO:1902042 | 24 | 6 | 1.91 | 0.0097595 | 2.01E+00 | negative regulation | BP | DOWN |
| | | | | | | of extrinsic | | |
| | | | | | | apoptotic signaling | | |
| | | | | | | pathway via death | | |
| | | | | | | domain receptors | | |
| GO:0045785 | 301 | 47 | 23.98 | 0.0097657 | 2.01E+00 | positive regulation | BP | DOWN |
| | | | | | | of cell adhesion | | |
| GO:0032355 | 134 | 19 | 10.67 | 0.0099102 | 2.00E+00 | response to estradiol | BP | DOWN |
| GO:2001020 | 134 | 19 | 10.67 | 0.0099102 | 2.00E+00 | regulation of | BP | DOWN |
| | | | | | | response to DNA | | |
| | | | | | | damage stimulus | | |
| GO:0050778 | 398 | 95 | 31.7 | 0.0099412 | 2.00E+00 | positive regulation | BP | DOWN |
| | | | | | | of immune response | | |
| GO:0043066 | 793 | 97 | 63.16 | 0.0099523 | 2.00E+00 | negative regulation | BP | DOWN |
| | | | | | | of apoptotic process | | |

TABLE 65
Gene Symbols for Enriched BIG-C Categories Increased by the HDAC6 Inhibitor

| | | Endosome | | | | | |
|---|---|---|---|---|---|---|---|
| | | and | | Fatty Acid | | ROS | Cell |
| Cytoskeleton | Transporters | Vesicles | Mitochondria | Biosynthesis | Peroxisomes | Protection | Surface |
| Sgce | Slc6a1 | Syt3 | Gpat2 | Echdc2 | Gstk1 | Prdx2 | Prnp |
| Mapt | Slc51a | Syngr1 | Gls2 | Acss2 | Nudt12 | Txnrd2 | Tspan6 |
| Krt18 | Kcnk12 | Rab3b | Amt | Hadh | Hacl1 | Gstp1 | Ptpru |
| Lrch2 | Slc27a6 | Snx31 | Cyp11a1 | Mecr | Pex11a | Prdx6 | Pcdh7 |
| Nphp1 | Slc12a5 | Clstn1 | Nme4 | Decr1 | Paox | | Folr1 |
| Pfn4 | Atp4a | Syt5 | Dhtkd1 | Pcx | Pex6 | | Cdh22 |
| Spata7 | Slc6a13 | Sytl1 | Me3 | | Pex11b | | Dlk1 |
| Dync2li1 | Kcnab3 | Unc13b | Nmnat3 | | Pex7 | | Adora3 |
| Kif17 | Slc22a18 | Pacsin3 | Maoa | | Pxmp4 | | Mc1r |
| Spire2 | Kcnh3 | Spag8 | Clybl | | Pex5 | | Tmem205 |
| Ang | Kcnh2 | Scg5 | Tmlhe | | | | Sema6c |
| Snph | Slc22a17 | Prss16 | Slc25a23 | | | | Adgrd1 |
| Krt86 | Slc7a4 | Fam109b | Tdrkh | | | | Ms4a3 |
| Mapre3 | Aqp9 | Vamp5 | Ldhd | | | | Smo |
| Dlg4 | Abcg2 | Rab17 | Hmgcs2 | | | | Adgrg7 |
| Homer2 | Fxyd1 | Rab38 | Fahd1 | | | | Ppfia3 |
| Eda | Slc44a5 | Ocrl | Bphl | | | | Ramp3 |
| Myo1d | Kcnd1 | Slc9a9 | Chchd6 | | | | Cd248 |
| Actn2 | Cacna1c | Snx22 | Aldh5a1 | | | | Gpr27 |
| Plekhg4 | Tfr2 | Dennd6b | Mthfd2l | | | | Muc13 |
| Ptpdc1 | Cacng8 | Arl4c | Acad10 | | | | Lrfn1 |
| Kif7 | Aqp11 | Tmem9 | Pyroxd2 | | | | Cadm3 |
| Tnni1 | Aqp1 | Tmem163 | Slc25a35 | | | | Plxna2 |
| Ank3 | Trpm1 | Sytl3 | Hint2 | | | | Pth1r |
| Tubb3 | Kcng2 | Stx2 | Bckdhb | | | | Epor |
| Ift81 | Slc2a10 | Appl2 | Lipt1 | | | | Cysltr1 |
| Gpr4 | Abcb4 | Rab27b | Coq4 | | | | Adgrl1 |
| Dock6 | Kcnh7 | Stxbp1 | Nipsnap1 | | | | Tspan17 |
| Ttc8 | Clcn1 | Abca5 | Cyp27a1 | | | | Unc5a |
| Ttc12 | Slc27a1 | Mamdc4 | Mccc2 | | | | Adam33 |
| Wdr35 | Clcn2 | Rab23 | Aldh4a1 | | | | Chrne |
| Epb41l4b | Slc38a5 | Als2cl | Pccb | | | | Lsr |
| Ehbp1 | Kcne3 | Scrn2 | Iba57 | | | | Enpp5 |
| Spag4 | Slc39a8 | Sft2d3 | Ppox | | | | Lhfpl2 |
| Odf2l | Kcnip3 | Ap4b1 | Glrx5 | | | | Tspan33 |
| Mylpf | Akap7 | Lamtor2 | Amacr | | | | Adgrg1 |
| Ift43 | Kcnc3 | Rab24 | Ethe1 | | | | Mmp17 |
| Nefh | Kctd14 | Hap1 | Acp6 | | | | Baiap2l2 |
| Wdr60 | Slc41a3 | Flot2 | Lyrm1 | | | | Cldn10 |
| Ttll1 | Aqp3 | Dennd1a | Sfxn2 | | | | Cdh24 |
| Dennd2a | Tesc | Rab3d | Dguok | | | | Arvcf |
| Wdr19 | Slc16a7 | Fcho1 | Agk | | | | Tmem41a |
| Vill | Slc14a1 | Rabep2 | Sfxn4 | | | | Sema6b |
| Kptn | Slc9a5 | Vps16 | Mcee | | | | Ptger3 |
| Pls3 | Kcnj8 | Ap1b1 | Immp2l | | | | Cd34 |
| Nek3 | Cacna1a | Ap1g2 | Clpb | | | | Neo1 |
| Bbs2 | Cnga1 | | Ivd | | | | Eng |
| Kifc2 | Spns3 | | Mtfr1l | | | | F11r |
| Bbs1 | Slc29a2 | | Naxe | | | | Palm |
| Dmd | Slc43a1 | | Adck5 | | | | Fgfr1 |
| Arl6 | Slc4a8 | | Sfxn5 | | | | Mfap3l |
| Dzip1 | Slc16a5 | | Pcca | | | | Gpr82 |
| Fuz | Slc29a4 | | Coq7 | | | | Hrh1 |
| Fnbp1l | Cbarp | | Ppa2 | | | | Gprc5c |
| Rpgr | Bspry | | Akap1 | | | | Agrn |
| Bbs9 | Slco2b1 | | Mccc1 | | | | Art3 |
| Tubb4a | Stom | | Acadsb | | | | Amot |
| Kif9 | Cacna2d2 | | Ccdc58 | | | | Plscr3 |
| Ccdc14 | Cacnb1 | | Slc25a24 | | | | Plpp2 |
| Palld | Slc29a1 | | Fxn | | | | Sdc2 |
| Bbs4 | Ano10 | | Suox | | | | Spa17 |
| Nek8 | Slc39a4 | | Acad11 | | | | Gpc4 |
| Krt10 | Slc2a9 | | Slc25a39 | | | | Plxdc2 |
| Arhgap18 | Slco3a1 | | Pstk | | | | Tmem17 |
| Ift74 | Kctd12 | | Acad8 | | | | Tacstd2 |
| Hook2 | Atp9a | | Fpgs | | | | Il1rl2 |
| Dctn6 | Slc50a1 | | Coq2 | | | | Anxa9 |
| Tubg2 | Slc19a1 | | Timm10b | | | | Sema4f |
| Eml2 | Kctd2 | | Tk2 | | | | Efna1 |
| Ccdc114 | Kctd13 | | Taz | | | | Cpne2 |
| Gsn | Slc39a3 | | Mipep | | | | Fgfrl1 |
| Cnn3 | Ttyh3 | | Dhodh | | | | Ephb6 |
| Matk | Slc6a20a | | Adck1 | | | | Adgra2 |
| Katnal1 | Ank | | Abcb8 | | | | Reck |
| Cep72 | Cbarp | | 2310061I04Rik | | | | Magi2 |
| Mks1 | Gm44509 | | 1700021F05Rik | | | | Grik5 |
| Klc4 | | | | | | | Gp5 |
| Pick1 | | | | | | | Tbxa2r |
| Kifap3 | | | | | | | Ramp1 |
| Cep57l1 | | | | | | | Plxna3 |
| Sgcb | | | | | | | Tgfb3 |
| Klhdc1 | | | | | | | Mpp7 |
| Ift122 | | | | | | | Tmem273 |
| Lrrc45 | | | | | | | Tlcd2 |
| Vmac | | | | | | | Gpr19 |
| Fntb | | | | | | | Ly6d |
| Sfi1 | | | | | | | Plscr4 |
| Cep41 | | | | | | | Prr7 |
| Tube1 | | | | | | | Adgr5 |
| Spata6 | | | | | | | Tmem107 |
| Cep19 | | | | | | | Ptprs |
| Mob3b | | | | | | | Smim13 |
| Cep131 | | | | | | | Krtcap3 |
| Fhl3 | | | | | | | Ano8 |
| Tuba4a | | | | | | | Gphn |
| Ccdc61 | | | | | | | Tjp3 |
| Ick | | | | | | | Lpar6 |
| Ift27 | | | | | | | Smim19 |
| Ip6k2 | | | | | | | Adora2a |
| Marveld1 | | | | | | | Amigo2 |
| Ankra2 | | | | | | | Ncstn |
| Pdlim1 | | | | | | | Tmem8a |
| Tpm1 | | | | | | | Cercam |
| Tubb4a | | | | | | | |

TABLE 66
Gene Symbols for Enriched BIG-C Categories Decreased by the HDAC6 Inhibitor

| | | | IFN gene | Pro | Unfolded | Endosome | | | |
|---|---|---|---|---|---|---|---|---|---|
| | Intracellular | PRR | Signature | Cell | Protein | and | Endoplasmic | | Integrin |
| Cell Surface | Signaling | Signaling | (IGS) | Cycle | & Stress | Vesicles | Reticulum | Golgi | Signaling |
| Ms4a4b | Dusp4 | Tlr13 | Mx1/Mx2 | Cep55 | Chac1 | Dnm3 | Edem1 | Fam20c | Gna13 |
| (includes | | | | | | | | | |
| others) | | | | | | | | | |
| Ctla2a/Ctla2b | Gnaz | Oasl2 | Ifi44 | Ncapg | Bhlha15 | Zfyve9 | Edem3 | Mest | Lamc1 |
| Igkv5-39 | Dusp14 | Oas2 | Rsad2 | Nek2 | Xbp1 | Itsn2 | Edem2 | Slc9a7 | Itgav |
| Ighv1-37 | Csrp3 | Tlr8 | Rtp4 | Cep85l | Edem1 | Ehd4 | Erp44 | Atp7a | Plcg2 |
| Ighv5-16 | Gng11 | Tlr13 | Eif2ak2 | Cdca2 | Creb3l2 | Dab2 | Erlec1 | Cgnl1 | Spock2 |
| Cd300ld | Rgs4 | Oas3 | Ifitm3 | Prc1 | Derl3 | Smap1 | Creld2 | B3gnt9 | Raf1 |
| Ace | Evc | Tlr7 | Sp110 | Ndc80 | Sel1l | Capza1 | Ddn | Plagl1 | Col17a1 |
| Ighv1-31 | Pik3ap1 | Zbp1 | Gbp2 | Foxm1 | Hspa5 | Washc4 | Ryr3 | Chst2 | Actg2 |
| Igkv12-41 | Rgs13 | Dusp16 | Sp100 | Ect2 | Edem3 | Eea1 | Hsp90b1 | Fgd4 | Lamc3 |
| Ighv1-33 | Aicda | Irf7 | Cmpk2 | Clspn | Dnajc3 | Arfgap3 | Sec24d | B3galt1 | Bcar1 |
| Ighv1-84 | Gzmk | Oasl2 | Ifit1 | Ttk | Insig1 | Snx2 | Kcnrg | Chst3 | Itgb8 |
| Ighv1-20 | Fkbp11 | Tirap | Ifit3b | Esco2 | Ube2j1 | Arpc1b | Tram2 | Glcci1 | Lama1 |
| Cx3cr1 | Sik2 | Isg15 | | Bub1 | Edem2 | Arap1 | Txndc11 | Ica1l | Col5a2 |
| Ighv5-9 | Foxp3 | Irf8 | | Dsn1 | Dnajb9 | Cyth4 | Txndc5 | Fndc3b | Hspg2 |
| Ighv1-42 | Rgs16 | Nlrc3 | | Top1 | Erp44 | Rab5a | Sdf2l1 | Xylt1 | Fbn2 |
| Ighv1-39 | Nos2 | Irf4 | | Fen1 | Hsph1 | Vps26a | Hyou1 | Chst1 | Eln |
| Crybg3 | Acod1 | Nlrc5 | | Kntc1 | Rpn1 | Arfgap2 | Sec24a | Fut8 | Col4a1 |
| Ighv1-26 | Zc3h12d | Tlr9 | | Ncaph | Herpud1 | Git1 | Prr11 | Man2a1 | Itgae |
| Ighv5-4 | Dusp3 | Irak4 | | Aurkb | Man1b1 | Rab35 | Hspa13 | Slc39a7 | Vcan |
| Ms4a4a | Tnip3 | Trim14 | | Ska3 | Vmp1 | Vps26b | Dnajb11 | Rab43 | Col4a2 |
| Tgm3 | Sh3bgrl2 | Lrrfip1 | | Cdc20 | Ubxn4 | | Manf | Manea | Col3a1 |
| Ighv5-12 | Amotl1 | Ticam2 | | Cenpe | Pdia4 | | Sec61a1 | Atp8b2 | Col13a1 |
| Ighv5-6 | Sh2b2 | Irf9 | | Kif11 | Tmem214 | | Pdia6 | Gcnt2 | Spon1 |
| Ighv2-9-1 | Spry1 | Ddx58 | | Cdca5 | Calr | | Atp2a2 | Parp9 | Ecm1 |
| Ighv2-9 | Nfil3 | Nlrp3 | | Top2a | Atf6 | | Lrrc59 | Cyb5d1 | |
| Ackr4 | Cmip | Tnfaip3 | | Cdc42bpb | Erlec1 | | Mtdh | Rab39b | |
| Ighv1-5 | Ikzf3 | Tlr4 | | Bub1b | Canx | | Tor3a | Bhlhe40 | |
| Ighv5-9-1 | Tiam1 | Casp4 | | Uhrf1 | Sec63 | | Ssr4 | B4galt5 | |
| Igkv3-2 | Parp14 | Ifih1 | | Incenp | Vcp | | Pdia3 | Qpctl | |
| Ighv3-4 | Prdm1 | Nod2 | | Spdl1 | St13 | | Slc35e1 | Itpripl2 | |
| Ighv5-15 | Bcl6 | Zc3hav1 | | E2f2 | Nploc4 | | Slc35b1 | Uso1 | |
| Clec4e | Styx | Traf3 | | Cdc45 | Ero1l | | Atp10d | Mgat2 | |
| Ighv1-36 | Themis2 | Arl16 | | Nusap1 | Ero1lb | | Plod2 | Cog5 | |
| Ighv1-21 | Trib1 | Rnf41 | | Dbf4 | | | Ergic1 | Serinc5 | |
| Ighv1-66 | Grk3 | Irak3 | | Sgo1 | | | Sec23a | Tpst1 | |
| Ighj3 | Stard8 | Myd88 | | Mis18bp1 | | | Surf4 | Sec14l1 | |
| Efnb2 | Ppp1r12a | Trim35 | | Espl1 | | | Sec11c | Slc30a7 | |
| Ccr6 | Fgr | Tank | | Cenpl | | | Mlec | Tmed5 | |
| Ighd2-7 | Rps6ka2 | Ifi213 | | Ccnd2 | | | Rrbp1 | Pask | |
| Gcsam | Irak1bp1 | | | Cep57 | | | Erap1 | Gcnt1 | |
| Ighv1-19 | Dck | | | Lats2 | | | Lclat1 | Pdxdc1 | |
| Igkv4-51 | Ikzf2 | | | E2f3 | | | Slc33a1 | Psen1 | |
| Ighv8-4 | Shcbp1 | | | Mcm6 | | | Ssr3 | Rnf157 | |
| Ighv1-73 | Lat2 | | | Cep68 | | | Ttc9c | G2e3 | |
| Lepr | Mzb1 | | | Cdk14 | | | Ergic2 | Ddhd1 | |
| Ighv1-7 | Prkab2 | | | Rfc1 | | | Stt3a | B3gnt5 | |
| Gjb2 | Socs3 | | | Ccnd1 | | | Sqle | Man1a2 | |
| Ighv1-28 | Ccdc88c | | | Cdc25b | | | Alg2 | Syngr2 | |
| Igkv5-45 | Dusp5 | | | Mcm5 | | | Elovl5 | St6galnac4 | |
| Ighv1-61 | Rasgrp3 | | | Mis12 | | | Clptm1l | Gsap | |
| Ighj1 | Prkcd | | | Helb | | | Ssr1 | Arcn1 | |
| Ighv1-18 | Batf | | | Mcmbp | | | Gdap2 | St8sia4 | |
| Ighg3 | Map3k8 | | | Cenpj | | | Sec23b | Tmf1 | |
| Igkv1-99 | Crkl | | | Cdc27 | | | Spcs3 | Bicd2 | |
| Igkv17-121 | Phlpp1 | | | Plk3 | | | Trim59 | Gga2 | |
| Ighv8-11 | Ptpn1 | | | Nek7 | | | Mia2 | Pde4dip | |
| Ighv1-58 | Stat3 | | | Pold1 | | | Srp72 | Slc38a10 | |
| Ptgir | Samsn1 | | | Nde1 | | | Soat1 | Cnst | |
| Ighv7-3 | Pou2af1 | | | Mcm4 | | | Yipf5 | Alkbh5 | |
| Ighv1-47 | Pptc7 | | | Ccng1 | | | Kctd20 | Copb2 | |
| Ighv1-75 | Rel | | | Ccnb1-ps | | | Dnajc14 | Rab30 | |
| Ighv1-12 | Nfam1 | | | Cenpc1 | | | Sec23ip | C1galt1 | |
| S1pr2 | Mob3a | | | C33002- | | | Stim2 | Fam20b | |
| | | | | 7C09Rik | | | | | |
| Cd300e | Pten | | | | | | Aldh3a2 | Chpf2 | |
| Ighv1-63 | Pla2g7 | | | | | | Ankrd13c | Tm9sf3 | |
| Ighv1-83 | Malt1 | | | | | | Ero1l | Slc30a5 | |
| Ulbp1 | Rnf19b | | | | | | Ero1lb | Gorasp2 | |
| Ighv2-2 | Prkca | | | | | | Deaf1 | Fut11 | |
| Igkv4-57-l | Bach1 | | | | | | Cyp51 | Osbpl9 | |
| Ighv1-25 | Nfkbie | | | | | | Rab1 | Atp2c1 | |
| Cd300lb | Btk | | | | | | | Copb1 | |
| Igkv2-116 | Mef2c | | | | | | | Vps54 | |
| Ighv6-6 | Ptpn11 | | | | | | | Gosr2 | |
| Igkv9-124 | Casp8ap2 | | | | | | | Copa | |
| Pcdhgc3 | Nfkbiz | | | | | | | Stip1 | |
| Ighv1-22 | Klhl6 | | | | | | | Copg1 | |
| Ighv1-4 | Lpxn | | | | | | | Tmed10 | |
| Ighv1-21-1 | Stk40 | | | | | | | Arl1 | |
| Drd4 | Map3k5 | | | | | | | Calu | |
| Ighv7-2 | Stat2 | | | | | | | Pcsk7 | |
| Igkv10-94 | Map3k3 | | | | | | | Gdi2 | |
| Igkv8-24 | Ppp4r1 | | | | | | | Furin | |
| Ighv2-4 | Ibtk | | | | | | | Gpr107 | |
| Thbd | Stap1 | | | | | | | Gga1 | |
| Igkv9-123 | Tnip2 | | | | | | | Man1a | |
| Ighv8-5 | Syk | | | | | | | | |
| Klra2 | Pkn1 | | | | | | | | |
| Ighv2-7 | Ywhah | | | | | | | | |
| Sgpp2 | Rpsbkb1 | | | | | | | | |
| Clec4a3 | Spred1 | | | | | | | | |
| Ighv2-6-8 | Map3k14 | | | | | | | | |
| Igkv15-103 | Stat6 | | | | | | | | |
| Itgae | Dusp6 | | | | | | | | |
| Igkv4-63 | Bcar3 | | | | | | | | |
| S1pr5 | Grb2 | | | | | | | | |
| Ighv3-3 | Ikbkb | | | | | | | | |
| Emp2 | Crk | | | | | | | | |
| Igkv5-37 | Camkk2 | | | | | | | | |
| Lag3 | Nfkb2 | | | | | | | | |
| Vasn | Elk1 | | | | | | | | |
| Igkv3-1 | Rnf31 | | | | | | | | |
| Igkj5 | Nfkb1 | | | | | | | | |
| Ighv14-4 | Sh3bp5 | | | | | | | | |
| Cdhr5 | Pip5k1c | | | | | | | | |
| Igkv10-95 | Btbd10 | | | | | | | | |
| Igkv13-85 | Ppp4r2 | | | | | | | | |
| Tnfrsf1b | Mapk1ip1l | | | | | | | | |
| Ighv2-3 | Kras | | | | | | | | |
| Ighj2 | Tnfaip8 | | | | | | | | |
| Igkv8-30 | Hck | | | | | | | | |
| Lair1 | Abi1 | | | | | | | | |
| Bmp8a | Fyn | | | | | | | | |
| Igkv17-127 | Prkx | | | | | | | | |
| Fcer1a | Ptpn12 | | | | | | | | |
| Ighv1-62-3 | Grk2 | | | | | | | | |
| Ptpro | Gimap4 | | | | | | | | |
| Igkv3-4 | Pld4 | | | | | | | | |
| Ighv8-8 | Sash3 | | | | | | | | |
| Vsig4 | Stt3b | | | | | | | | |
| Ighv2-6 | Ikbkg | | | | | | | | |
| Igkv9-129 | Pxk | | | | | | | | |
| Ighv14-3 | Ptpn6 | | | | | | | | |
| Igkv4-69 | Csnk1g3 | | | | | | | | |
| Ighv6-5 | Ywhag | | | | | | | | |
| Igkv8-21 | Plaa | | | | | | | | |
| Igkv5-43 | Tesk1 | | | | | | | | |
| Igkv4-86 | Blk | | | | | | | | |
| Ighe | Mknk1 | | | | | | | | |
| Ceacam1 | Blnk | | | | | | | | |
| Igkv6-32 | Ppp6r1 | | | | | | | | |
| Ighg1 | Vav1 | | | | | | | | |
| Nt5e | Sav1 | | | | | | | | |
| Ighv10-1 | Lyn | | | | | | | | |
| Igkv3-11 | Csnk1a1 | | | | | | | | |
| Ighv3-6 | Ppp2r5c | | | | | | | | |
| Astl | Sh2d3c | | | | | | | | |
| Ighv1-14 | Ppm1f | | | | | | | | |
| Igkv4-73 | Stk24 | | | | | | | | |
| Igkv4-74 | Tnip1 | | | | | | | | |
| Igkv2-137 | Ppp2r5d | | | | | | | | |
| Igkv4-70 | Map3k11 | | | | | | | | |
| Ighv5-17 | Glyr1 | | | | | | | | |
| Mreg | 1700019D03Rik | | | | | | | | |
| Epha4 | Gm10031 | | | | | | | | |
| Ighv1-54 | H13 | | | | | | | | |
| Igkv6-14 | Calm1 (includes others) | | | | | | | | |
| Gpr35 | | | | | | | | | |
| Igkv6-29 | | | | | | | | | |
| Igkv6-23 | | | | | | | | | |
| Ighd1-1 | | | | | | | | | |
| Gja4 | | | | | | | | | |
| Ighv5-2 | | | | | | | | | |
| Igkv3-9 | | | | | | | | | |
| Lrp8 | | | | | | | | | |
| Ighv1-77 | | | | | | | | | |
| Igkv3-12 | | | | | | | | | |
| Lilra5 | | | | | | | | | |
| Nrcam | | | | | | | | | |
| Igkv12-46 | | | | | | | | | |
| Tigit | | | | | | | | | |
| Adgrg6 | | | | | | | | | |
| Ighv1-78 | | | | | | | | | |
| Ighv1-76 | | | | | | | | | |
| Ighv13-2 | | | | | | | | | |
| Ighv7-1 | | | | | | | | | |
| Igkv4-77 | | | | | | | | | |
| Ighv14-2 | | | | | | | | | |
| Ccr8 | | | | | | | | | |
| Ighv1-2 | | | | | | | | | |
| Igkj3 | | | | | | | | | |
| Efnb1 | | | | | | | | | |
| Ighv1-56 | | | | | | | | | |
| Slamf7 | | | | | | | | | |
| Muc20 | | | | | | | | | |
| Ighv8-2 | | | | | | | | | |
| Il1r2 | | | | | | | | | |
| Ighv1-30 | | | | | | | | | |
| Ighv1-43 | | | | | | | | | |
| Treml4 | | | | | | | | | |
| Ptprg | | | | | | | | | |
| Ms4a2 | | | | | | | | | |
| Cd244 | | | | | | | | | |
| Nrp1 | | | | | | | | | |
| Igsf11 | | | | | | | | | |
| Igkv2-109 | | | | | | | | | |
| Igkv4-61 | | | | | | | | | |
| Tnfrsf4 | | | | | | | | | |
| Tgm2 | | | | | | | | | |
| Cd83 | | | | | | | | | |
| Igkv8-28 | | | | | | | | | |
| Plxnb2 | | | | | | | | | |
| Lifr | | | | | | | | | |
| Clec1b | | | | | | | | | |
| Igkv4-58 | | | | | | | | | |
| Tnfsf8 | | | | | | | | | |
| Crybg3 | | | | | | | | | |
| Slamf1 | | | | | | | | | |
| Notch1 | | | | | | | | | |
| Cdh11 | | | | | | | | | |
| Msr1 | | | | | | | | | |
| Sdc1 | | | | | | | | | |
| Trabd2b | | | | | | | | | |
| Pdcd1 | | | | | | | | | |
| Ighv7-4 | | | | | | | | | |
| Adam19 | | | | | | | | | |
| Gngt2 | | | | | | | | | |
| Cdh5 | | | | | | | | | |
| Hmmr | | | | | | | | | |
| Jchain | | | | | | | | | |
| Igkv9-120 | | | | | | | | | |
| Havcr1 | | | | | | | | | |
| Ctla4 | | | | | | | | | |
| Pilra | | | | | | | | | |
| Kazn | | | | | | | | | |
| Colec12 | | | | | | | | | |
| Fut4 | | | | | | | | | |
| Susd2 | | | | | | | | | |
| C5ar1 | | | | | | | | | |
| Igkj2 | | | | | | | | | |
| Igkv1-122 | | | | | | | | | |
| P2ry12 | | | | | | | | | |
| Il13ra1 | | | | | | | | | |
| Igkv1-117 | | | | | | | | | |
| Ptger2 | | | | | | | | | |
| Gp9 | | | | | | | | | |
| Igsf6 | | | | | | | | | |
| Siglec1 | | | | | | | | | |
| Cldn7 | | | | | | | | | |
| Paqr5 | | | | | | | | | |
| Clec4d | | | | | | | | | |
| Fpr1 | | | | | | | | | |
| Tgfbr1 | | | | | | | | | |
| Fpr2 | | | | | | | | | |
| Il21r | | | | | | | | | |
| Notch3 | | | | | | | | | |
| Itgal | | | | | | | | | |
| Igkc | | | | | | | | | |
| Ptch1 | | | | | | | | | |
| Alcam | | | | | | | | | |
| Plxnd1 | | | | | | | | | |
| Ptprk | | | | | | | | | |
| Ldlrad3 | | | | | | | | | |
| Sema4d | | | | | | | | | |
| Gpm6b | | | | | | | | | |
| Csf1r | | | | | | | | | |
| Ptpre | | | | | | | | | |
| Ms4a6c | | | | | | | | | |
| Cd300a | | | | | | | | | |
| Scimp | | | | | | | | | |
| Icos | | | | | | | | | |
| P2ry14 | | | | | | | | | |
| Epcam | | | | | | | | | |
| Tnfrsf17 | | | | | | | | | |
| Ptafr | | | | | | | | | |
| Acvr1 | | | | | | | | | |
| Entpd1 | | | | | | | | | |
| Clec7a | | | | | | | | | |
| Ltbr | | | | | | | | | |
| Jmjd4 | | | | | | | | | |
| Adam9 | | | | | | | | | |
| Dcbld1 | | | | | | | | | |
| Itgax | | | | | | | | | |
| Rftn1 | | | | | | | | | |
| Jaml | | | | | | | | | |
| Cd180 | | | | | | | | | |
| Enpp1 | | | | | | | | | |
| P2ry6 | | | | | | | | | |
| Ptger4 | | | | | | | | | |
| Plekha2 | | | | | | | | | |
| Itgb3 | | | | | | | | | |
| Myo1g | | | | | | | | | |
| Icam1 | | | | | | | | | |
| Ldlr | | | | | | | | | |
| Cmklr1 | | | | | | | | | |
| Cd40 | | | | | | | | | |
| Adam17 | | | | | | | | | |
| Tnfrsf13c | | | | | | | | | |
| Pitpnb | | | | | | | | | |
| Adgre1 | | | | | | | | | |
| Cnr2 | | | | | | | | | |
| Ighd | | | | | | | | | |
| Cerk | | | | | | | | | |
| Pon2 | | | | | | | | | |
| Sdc3 | | | | | | | | | |
| Cd22 | | | | | | | | | |
| Susd6 | | | | | | | | | |
| Cpne3 | | | | | | | | | |
| Slamf6 | | | | | | | | | |
| Il17ra | | | | | | | | | |
| Sell | | | | | | | | | |
| Adam10 | | | | | | | | | |
| Cd38 | | | | | | | | | |
| Pqlc3 | | | | | | | | | |
| Tmem65 | | | | | | | | | |
| Tgfbr2 | | | | | | | | | |
| Itgb2 | | | | | | | | | |
| Sppl2a | | | | | | | | | |
| Ms4a6b | | | | | | | | | |
| Cd44 | | | | | | | | | |
| Tspan14 | | | | | | | | | |
| Igkv9-120 | | | | | | | | | |
| Igkv9-123 | | | | | | | | | |
| Igkv9-124 | | | | | | | | | |
| Igkv9-129 | | | | | | | | | |
| Iglc4 | | | | | | | | | |
| Igll1 | | | | | | | | | |
| Icam4 | | | | | | | | | |
| Fcgr4 | | | | | | | | | |
| Klra2 | | | | | | | | | |
| VASN | | | | | | | | | |
| Clec4n | | | | | | | | | |
| Csf2rb2 | | | | | | | | | |
| Fcgr1 | | | | | | | | | |
| Fcgr3 | | | | | | | | | |
| I14ra | | | | | | | | | |
| Gm15931 | | | | | | | | | |
| Lilrb4a | | | | | | | | | |
| Siglece | | | | | | | | | |
| Sirpb1c | | | | | | | | | |
| Ms4a6d | | | | | | | | | |
| Ttc7 | | | | | | | | | |
| Igip | | | | | | | | | |

FIGS. 110A-110C show a non-limiting example of results showing that HDAC6 inhibition decreased citrate synthase activity and cytochrome c oxidase activity in NZB/W mice. Four weeks of treatment of NZB/W mice with the HDAC6 inhibitor ACY-738 lead to a significant decrease in the rate limiting enzyme of the TCA cycle (p=0.043) (FIG. 110A), and a decrease in cytochrome C oxidase activity (P=0.053) (FIG. 110B), while having minimal effect on beta hydroxyacyl coa dehydrogenase in splenocytes (n=5) (FIG. 110C).

FIGS. 111A-111B show a non-limiting example of results showing that HDAC6 inhibition decreases glucose and fatty acid oxidation in T and B cells from NZB/W mice. T cells and B cells from 12-week old NZB/W female were purified and stimulated with anti CD3/CD28 or LPS respectively for 24 hours with or without the addition of 4 μM ACY-738 (DMSO only was used as control). After 24 hours of culture, CO₂ production from the oxidation of glucose (FIG. 111A) and palmitate (FIG. 111B) were determined from three separate experiments in triplicate (n=3).

FIG. 112 shows a non-limiting example of results showing that HDAC6 inhibition decreases lupus gene signature pathways in NZB/W mice that are increased in active human SLE. IPA canonical signaling pathways increased in human SLE microarray tissue datasets were compared to signaling pathways in NZB/W mice decreased by the HDAC6 inhibitor. Z scores greater or less than 2 are considered significant.

FIGS. 113A-113B show a non-limiting example of quantified germinal center formation in NZB/W female mice at 24 weeks-of age-treated with ACY-738 (treated, “T”) or without ACY-738 (control, “C”) for four weeks. We randomly picked 5 germinal centers from each spleen sample and analyzed by using ImageJ software to calculate the size of the germinal center. N=20, * P<0.05, **** P<0.0001.

FIGS. 114A-114D show a non-limiting example of results obtained by flow cytometry of GC B cells (FIGS. 114A and 114C) and TFH (FIGS. 114B and 114D) assessed by flow cytometry in C57BL/6J mice and C57BL/6J/HDAC6^{-/-} mice. For spleen, n=5 (FIGS. 114A-114B), and for Peyer's patch, n=3 (FIGS. 114C-114D). Germinal center B cells are gated by CD19+, GL7+, IgD−. * P<0.05.

FIGS. 115A-115F show a non-limiting example of results obtained by flow cytometry of sorted B cells from C57BL/6J mice and C57BL/6J/HDAC6^{-/-} mice stimulated with LPS or anti-IgM, anti-CD40 for 24 hours. The results showed reduced expression of activation markers of B cells CD86 (FIG. 115A) and MHCII (FIG. 115B) in C57BL/6J/HDAC6^{-/-} mice compared to C57BL/6J mice with stimulation of anti-IgM and anti-CD40. In addition, MFI of CD69 (FIG. 115C), CD86 (FIG. 115D), MHC-II (FIG. 115E), and CD80 (FIG. 115F) are down-regulated in C57BL/6J/HDAC6^{-/-} mice with stimulation of LPS. N=5. * P<0.05, ** P<0.01

FIGS. 116A-116F show a non-limiting example of results obtained by flow cytometry of sorted B cells from NZB/W mice stimulated with LPS or anti-IgM, anti-CD40 and then treated with ACY738 for 24 hours. The results showed reduced expression of activation markers of B cells CD86 (FIG. 116A) and MHCII (FIG. 116B) in ACY-738 treated B cells with stimulation of anti-IgM and anti-CD40. In addition, MFI of CD69 (FIG. 116C), CD86 (FIG. 116D), MHC-II (FIG. 116E), and CD80 (FIG. 116F) are significantly down-regulated in ACY-738 treated B cells with stimulation of LPS. N=5. * P<0.05, ** P<0.01, *** P<0.001, **** P<0.0001.

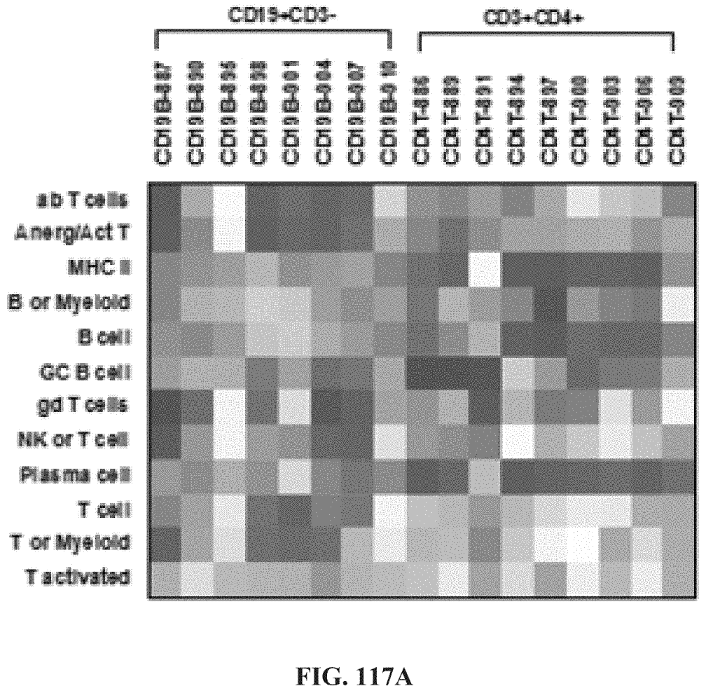

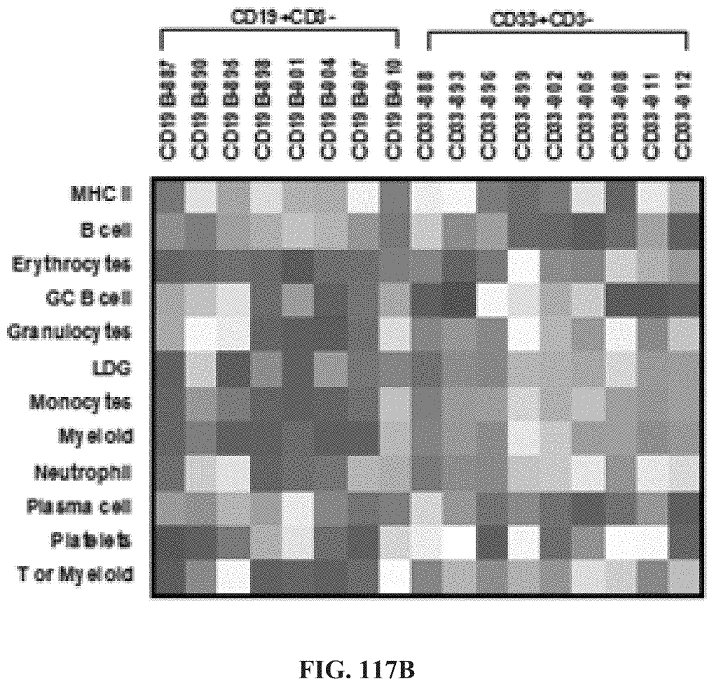

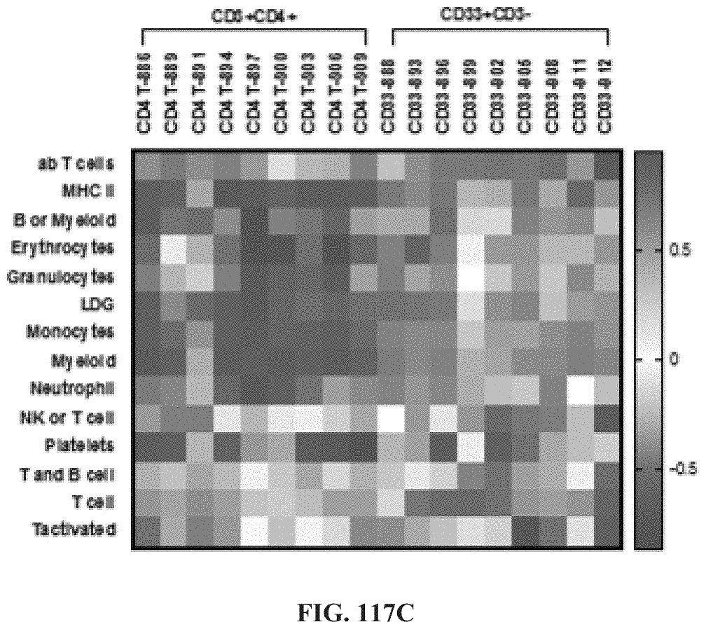

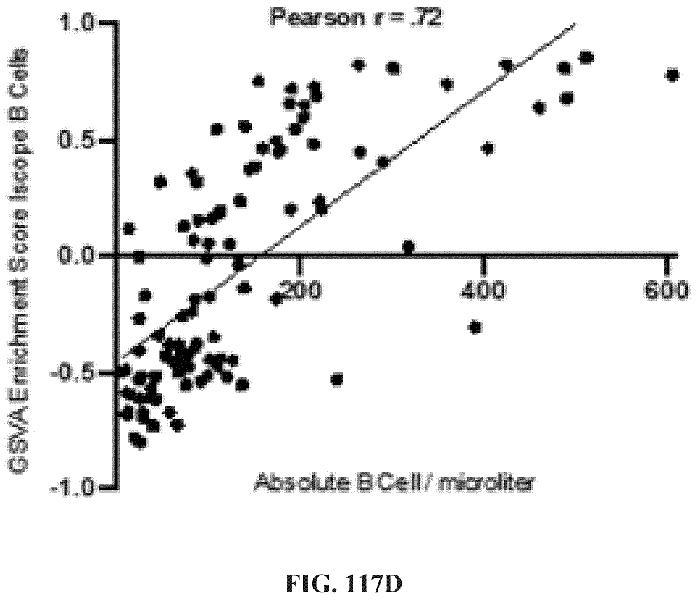

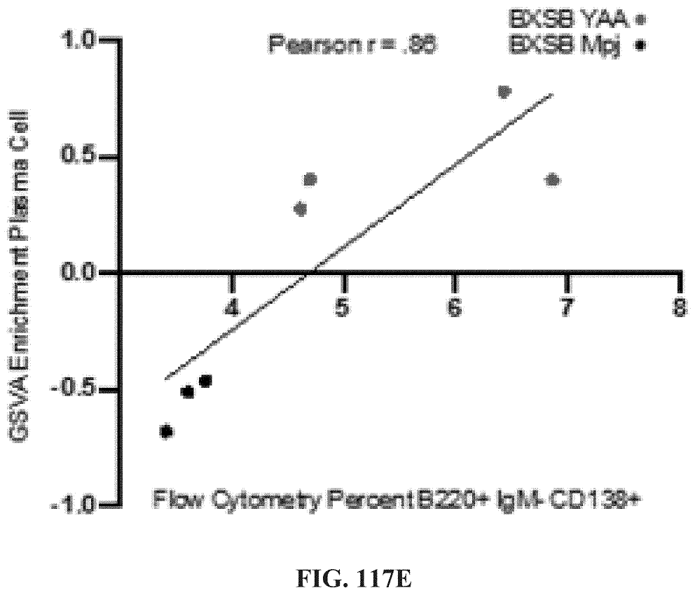

FIGS. 117A-117C show a non-limiting example of control experiments demonstrating the specificity and lack of cross reactivity of I-scope. Experiments were performed on the DE analysis of healthy control purified CD3+CD4+ T cells (FIGS. 117A and 117C), CD19+CD3− B and Plasma Cells (FIGS. 117A-117B), and CD33+CD3− Myeloid cells (FIGS. 117B-117C) from microarray dataset GSE10325. The genes in each I-scope category (29 categories in total; hematopoietic general was not used) were used as modules for gene set variation analysis to determine the specificity of each module and cross-reactivity to other cell types. For each comparison, only categories with at least three genes above the Interquartile Range threshold were considered for statistical analysis. Significance of GSVA enrichment scores was determined using Sidak's multiple comparisons test. Adjusted p values below 0.05 were considered significant. FIGS. 117D-117E show a non-limiting example of results demonstrating a strong relationship of human B cell/microliter counts to GSVA enrichment scores for the I-scope B cell category on 105 human subjects from microarray dataset GSE88884. A strong relationship was demonstrated of mouse flow cytometry values for plasma cells (B220+IgM-CD138+) and the GSVA enrichment scores using the I-scope plasma cell module on BXSB Yaa (points above X-axis) and BXSB MPJ mice (points below X-axis).

### Example 33: Translation and Interpretation of Mouse Genomic Information Toward Applications in Humans

Mouse models may serve an important role in understanding disease processes and may be vital for understanding the function of individual gene products. It may be important to understand how mouse and human genes relate to each other in order to properly use mouse models to understand mechanisms of disease and to predict new drug targets. Translating the expression of mouse genes into their proper human counterparts may be done by first determining if an ortholog for the human gene exists. This may be done using a variety of free programs such as biomaRt, DAVID NCIF, Homologene, and the appropriate ensemble identification for each gene. Although this allows the mapping of mouse to human orthologs, there may be at least two important challenges which may potentially hinder understanding of how to relate mouse to human disease and may be particularly problematic for the amount of data generated during genomic mouse and human studies.

The first challenge is the role of convergent evolution, which results in the presence of genes which are not orthologous between humans and mice, but that serve similar functions and have similar expression patterns between humans and mice. Examples of these genes include the hundreds of T cell receptor alpha, beta, gamma and delta chain genes, the immunoglobulin genes, the Major Histocompatibility Complex genes, and the NK cell inhibitory genes. Although these genes serve similar roles in mice and humans, and by their expression help identify specific cell types and processes, the genes do not map to each other using conventional methodologies.

The second challenge arises when genes are technically orthologous, but the function of the gene and its expression patterns are quite disparate between mice and humans. One example of these genes is the gene Arg1 in mice. Arg1 is an ortholog (83.13 percent similar) to human ARG1, but closer examination reveals that Arg1 in mice is overexpressed in mouse macrophages with anti-inflammatory capabilities, and human ARG1 may be only detected in mature neutrophils and may be associated with the immune response to fungi.

The present disclosure provides improved approaches for comparing mouse and human genomic data (e.g., for drug target assessment applications). The improved approaches may comprise use of methods and a database developed to improve mouse to human translation in an effort to better understand how mouse models can be used to improve understanding of human disease and predict better therapeutics.

As mentioned above, there may be two challenges encountered when trying to understand genomic information in the mouse and translate it to relevant information for human disease. Using methods and systems of the present disclosure, suitable algorithms may be used to interpret gene expression datasets in the human. Such algorithms (e.g., P-scope, BIG-C, I-scope, and T-scope) analyze gene expression data and generate sets of signaling pathways, processes, and cell types that are expressed in human diseases. These tools may be customized for application to mice models, based on the expression and function of the genes in the mouse. Mouse gene expression data are entered into the mouse versions of the P-scope, BIG-C, I-scope, and T-scope algorithms, and the signaling pathways, processes, and cell types that are enriched are determined. Because the mouse versions of P-scope, BIG-C, I-scope, and T-scope use information gleaned from mouse experiments, genes which may not have orthologues in humans are correctly placed in categories and the outputs may be compared directly to human genomic data. Additionally, genes which are indicative of different cell types, like Arg1, are placed in myeloid I-scope and anti-inflammatory signaling pathways in the mouse, thereby allowing a direct comparison to be made human myeloid and anti-inflammatory signaling pathways. This approach enables the signaling pathways and cells operating in mouse models of disease to be determined and then compared, translated, and/or interpreted toward human results.

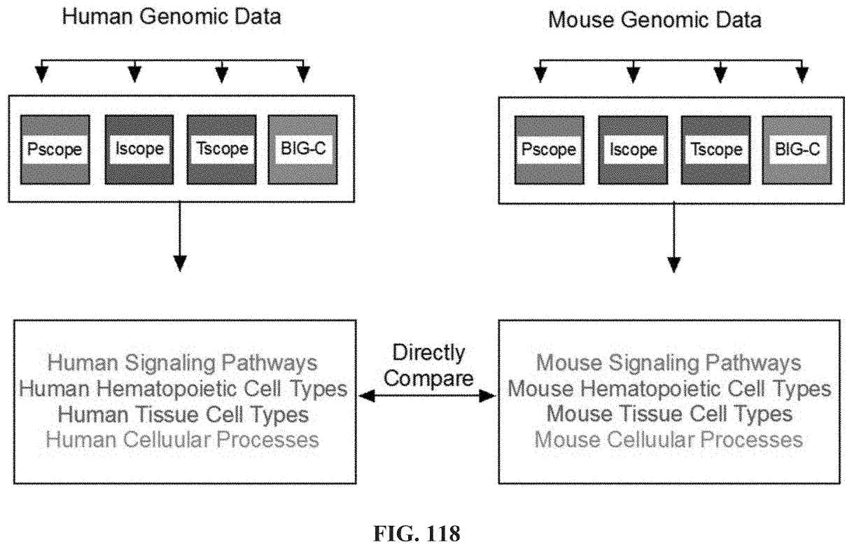

FIG. 118 illustrates an example of a process for translating mouse to human genomic data, which allows a direct comparison of human and mouse genomic data. A human genomic dataset is analyzed using one or more data analysis algorithms, such as P-scope, I-scope, T-scope, and BIG-C, as described elsewhere herein. Further, an animal (e.g., mouse) genomic dataset is analyzed using one or more data analysis algorithms, such as P-scope, I-scope, T-scope, and BIG-C, as described elsewhere herein. For example, the human genomic dataset may be analyzed by P-Scope to assess human signaling pathways, by I-Scope to assess human hematopoietic cell types, by T-Scope to assess human tissue/cell types, and/or by BIG-C to assess human cellular processes. Further, the mouse genomic dataset may be analyzed by P-Scope to assess human signaling pathways, by I-Scope to assess mouse hematopoietic cell types, by T-Scope to assess mouse tissue/cell types, and/or by BIG-C to assess mouse cellular processes. Then the data analysis results may be directed compared between human and mouse genomic data. For example, the P-Scope analysis of signaling pathways, the I-Scope analysis of mouse hematopoietic cell types, the T-Scope analysis of mouse tissue/cell types, and/or the BIG-C analysis of cellular processes may be compared between the human and mouse genomic data.

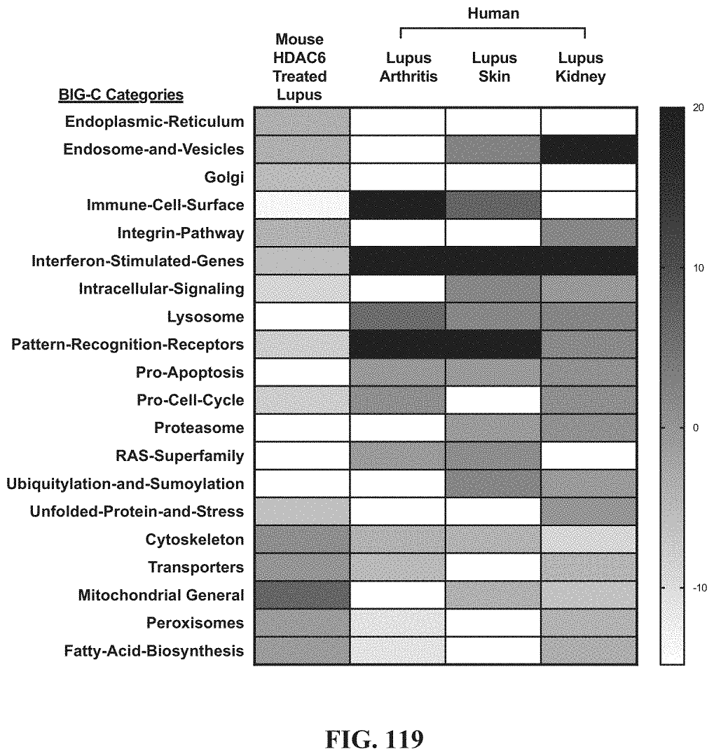

In FIG. 119, an example of this process is shown, using a BIG-C comparison of treated mouse lupus and human lupus tissue. In this experiment, mice with lupus were successfully treated with an HDAC-6 inhibitor to cure their disease, and gene expression was performed and analyzed using a BIG-C algorithm. This mouse analysis was directly compared to the BIG-C analysis of human lupus arthritis tissue, lupus skin tissue, and lupus kidney tissue, thereby demonstrating that inhibition of HDAC-6 alleviated many of the aberrant pathways operating in human lupus disease.

Further, the improved approaches for comparing mouse and human genomic data (e.g., for drug target assessment applications) may comprise developing a database of “true ortholog” genes. Such pairs of genes (each comprising mouse gene and one human gene) may be both orthologous pairs and have similar function and gene expression patterns (e.g., among mice and humans with active autoimmune disease, such as lupus).

The development of the mouse versions of P-scope, I-scope, T-scope, and BIG-C algorithms may comprise extensive literature mining and determining whether mouse genes orthologous to human genes also have similar cellular expression and function (thereby being “true orthologs” to human genes). During this process, a database is created of mouse and human orthologues that have similar cellular expression and function. This database takes into account results of published human and mouse studies to determine if genes which share homology also have similar expression patterns and function. Table 67 lists several examples of genes that are orthologous in human and mouse, and whether or not they meet the criteria to be considered “true orthologs.” This database may enable quick determination of whether drug targets are practical to target in mice for evidence in humans.

TABLE 67
Examples of “true ortholog” genes in a database

| | | AMPEL TRUE | | |
|---|---|---|---|---|
| Gene in Human | Ortholog in Mouse | ORTHOLOG | WHY? | Reference |
| ARG1 | Arg1 | No | Expression on different cell types | Pillay, 2013 |
| | | | between human and mouse. Involved in | |
| | | | different functions between human and | |
| | | | mouse | |
| TLR9 | Tlr9 | No | In human expression is restricted to B | Lund, 2003 |
| | | | cells and pDC, in mouse it is expressed on | |
| | | | all myeloid cells | |
| CD4 | Cd4 | No | Expressed on human macrophages and | Crocker, 1987 |
| | | | the helper T cell subset. Only expressed | |
| | | | on T cells in mice. | |
| CD33 | Cd33 | No | Expressed on human myeloid cells, only | Brinkman, 2003 |
| | | | expressed on granulocytes in mice. | |
| CD38 | Cd38 | No | High on germinal center B cells and | Gordon, 2001 |
| | | | plasma cells in human; in mouse it is low | |
| | | | on germinal center B cells and not | |
| | | | expressed on human plasma cells. | |
| VCAM1 | Vcam1 | Yes | Expressed on similar cells. Similar | Yednock, 1992 |
| | | | function. | |
| CD247 | Cd247 | Yes | Similar expression pattern and function | Swarmy, 2007; Alarcon, 2006 |
| CD3D | CD3d | Yes | Similar expression pattern and function | Swarmy, 2007; Alarcon, 2006 |
| CD3G | Cd3g | Yes | Similar expression pattern and function | Swarmy, 2007; Alarcon, 2006 |
| JAK2 | Jak2 | Yes | Similar expression pattern and function | Park, 2013 |
| VWF | Vwf | Yes | Similar expression pattern and function. | Pergolizzi, 2006 |

### Example 34: Identification of Alterations in Macrophage Activation Associated with Disease Activity in Systemic Lupus Erythematosus (SLE)

Systemic lupus erythematosus (SLE) may be characterized by abnormalities in B cell and T cell function, but the role of disturbances in the activation status of macrophages (Mϕ) may not be well described in human patients. Recognizing this need, gene expression profiles from isolated lymphoid and myeloid populations were analyzed to identify differentially expressed (DE) genes between healthy controls and patients with either inactive or active SLE. While hundreds of DE genes were identified in B and T cells of active SLE patients, there were no DE genes found in B or T cells from patients with inactive SLE compared to healthy controls. In contrast, large numbers of DE genes were found in myeloid cells (MC) from both active and inactive SLE patients. Among the DE genes were several that may play roles in Mϕ activation and polarization, including the M1 genes STAT1 and SOCS3 and the M2 genes STAT3, STATE, and CD163. M1-associated genes were far more frequent in data sets from active versus inactive SLE patients. To characterize the relationship between Mϕ activation and disease activity in greater detail, weighted gene co-expression network analysis (WGCNA) was performed to identify modules of genes associated with clinical activity in SLE patients. Among these genes were disease activity-correlated modules containing activation signatures of predominantly M1-associated genes. No disease activity-correlated modules were enriched in M2-associated genes. Pathway and upstream regulator analysis of DE genes from both active and inactive SLE MC were cross-referenced with high-scoring hits from the drug discovery Library of Integrated Network-based Cellular Signatures (LINCS) to identify new strategies to treat both stages of SLE. A machine learning approach employing MC gene modules and a generalized linear model was performed to predict the disease activity status in unrelated gene expression data sets.

In summary, altered MC gene expression is characteristic of both active and inactive SLE. However, disease activity is associated with an alteration in the activation of MC, with a bias toward the M1 proinflammatory phenotype. These data demonstrate that while hyperactivity of B cells and T cells is associated with active SLE, MC potentially direct flare-ups and remission by altering their activation status toward the M1 state.

SLE may be typically characterized by B cell hyperactivity and autoantibody formation, promoted by T cell dysregulation. The role of MC in SLE, however, may remain poorly understood despite their considerable influence on adaptive immunity. Mϕ and dendritic cells (DCs) are phagocytic professional antigen presenting cells (APC) of myeloid lineage that may be integral to the propagation and orchestration of immune responses. Although DCs may be the main myeloid cell (MC) population responsible for antigen presentation, phagocytosed antigens may also be processed by Mϕ and presented on the Mϕ surface by MHC-I and -II molecules to activate both B cells and T cells.

Bone marrow (BM)-derived Mϕ may originate from hematopoietic stem cells (HSC) that differentiate into common myeloid progenitor (CMP) cells and subsequently into monocytes. Upon activation, patrolling monocytes may further differentiate into Mϕ to address the injury or infection they have detected. DCs may also originate from myeloid progenitors, specifically from the common DC progenitor (CDP) which develops from the CMP along with monocytes. The CDP may give rise to both plasmacytoid DCs (pDC) and pre-DCs, which give rise to classical DCs (cDC). pDCs, which may be identified by expression of B220, Siglec-H, and Bst2, may be less phagocytic and less efficient APC and instead may be responsible for producing large amounts of type I interferon to combat viral infections.

Mϕ may express a large collection of surface receptors to monitor their local microenvironment that allows them to act as sentinels for markers of infection or injury. Engagement of these receptors by cell debris, viral or bacterial byproducts, cytokine and chemokine signals, and other factors may activate Mϕ and allow them to modify their phenotype and function rapidly and contribute to host defense. Mϕ may combat infectious disease both through intracellular destruction of phagocytosed pathogens and via production of various antimicrobial peptides, reactive oxygen intermediates, and nitric oxide. Other innate functions of activated Mϕ may include wound repair and tissue remodeling, and proinflammatory Mϕ may eliminate tumor cells in the early stages of cancer. As early responders at sites of inflammation and infection, Mϕ may also shape the early adaptive immune response by reacting to changes in the microenvironment and secreting various chemokines and cytokines to recruit other immune cells.

Specific stimulating factors and signals may cause Mϕ to undergo extreme changes in transcriptional regulation and assume a specific activation state ranging from highly proinflammatory to anti-inflammatory in a process called Mϕ polarization. Each polarization state or subset may express a particular profile of surface receptors, cytokines, chemokines, and secreted effector molecules that dictates its functional effect on inflammation, immune cell recruitment and activation (or suppression), and tissue remodeling. Named in accordance with the Th1/Th2 paradigm of immune responses, the M1 and M2 polarization states may represent canonical proinflammatory and anti-inflammatory Mϕ functional states, respectively, and indeed, may produce cytokines and chemokines that correspond to Th1 and Th2 response induction. The whole of Mϕ polarization, however, may represent a spectrum of overlapping phenotypic states between M1 and M2 Mϕ, and several other subsets between these extremes may be defined in various disease models.

There may be growing appreciation for the contribution of Mϕ polarization to both disease progression and resolution. Alteration of the M1/M2 Mϕ balance may be shown to have crucial roles in bacterial and viral infections, and many pathogens have evolved escape mechanisms that manipulate Mϕ polarization to enhance their survival and spread. M1 and M2 Mϕ may also influence local inflammation, the dysregulation of which is central to the pathology of diseases with inflammatory components, including type 1 diabetes, obesity, non-alcoholic steatohepatitis, atherosclerosis, and Crohn's disease. The contribution of Mϕ to SLE-like disease pathogenesis may be explored in mice, but a lack of human studies may hinder the investigation of activated Mϕ as potential contributors to molecular pathology and as therapeutic targets. Recognizing this need, a bioinformatics-based approach was employed to examine the myeloid-derived genomic signatures that define both active and inactive SLE in human patients and to identify promising candidates empirically for drug intervention.

Selection, quality control, and normalization of raw data files were performed as follows. Raw data files for human peripheral myeloid cells purified from SLE patients and healthy controls (HC) were obtained from the publicly accessible Gene Expression Omnibus (GEO) repository (CD33+ cells [GSE10325; 10HC, 7 active SLE] and CD14+ cells [GSE38351; 12HC, 8 active SLE, 5 inactive SLE]). SLE patients with an SLE Disease Activity Index (SLEDAI) score less than six were defined as having inactive disease, whereas those with a SLEDAI score of 6 or greater were defined as having active disease. Raw data files for T and B cells isolated from SLE patients or HCs were obtained from GEO to be used for later comparative analyses (GSE10325 [CD4+ T cells, CD19+ B cells], GSE51997 [active CD4+ T cells], and GSE4588 [active CD19+ B cells]).

Processing of raw data files, obtained for each respective study on GEO, was performed with Bioconductor packages GEOquery, affy, affycoretools, and simpleaffy in R. Raw array data were inspected for visual artifacts or poor RNA hybridization using Affymetrix QC plots. Datasets that passed quality control measures were normalized using the GCRMA method (guanine cytosine robust multiarray averaging), and transformed to obtain log 2 intensity values, which were formatted into R expression set objects (E-sets). Principal component analysis (PCA) plots were generated for all cell types in each experiment to inspect for outlier samples, admixed disease cohorts, and batch effects visually.

Raw microarray data were annotated using chip definition files (CDF) appropriate to the microarray product from Affymetrix. In order to identify additional genes unrecognized by Affymetrix CDFs, the same data were subsequently processed and annotated using custom BrainArray CDF version 19. Probe sets lacking annotations by the Affymetrix CDF were interrogated for BrainArray definitions. Any probes that were annotated by Affymetrix CDF but also were incorporated in BrainArray probe sets identifying alternative genes were excluded. For Affymetrix HGU133A platform microarrays, a total of 12,504 genes were identified by Affymetrix CDF. Of these, 11,825 were also identified by BrainArray and an additional 354 genes were identified by BrainArray alone, whereas 143 Affymetrix probe sets were excluded.

Differential gene expression (DE) analysis was performed as follows. The annotated E-sets were filtered to remove probes with very low intensity values via visual operator selection of thresholds set at the trough of low intensity histogram frequencies, post-normalization. Any probes that lacked gene annotation data were also discarded. GCRMA normalized expression values were variance corrected using local empirical Bayesian shrinkage before calculation of DE using the ebayes function in the Bioconductor LIMMA package. Resulting p-values were adjusted for multiple hypothesis testing using the Benjamini-Hochberg correction, which reports a false discovery rate (FDR). Probe sets within each study were filtered to retain differentially expressed (DE) probes with an a priori FDR of less than 0.2, which were considered statistically significant. This FDR cutoff was employed with the understanding that additional false positive probes may be included in the analysis but that fewer false negative probes may then be inappropriately excluded. Since additional analyses that did not involve an estimate of FDR were included to confirm the results and exclude the contributions of false positives, there was greater concern about excluding apparent false negatives from the analysis. This list was further filtered to retain only the most significant probe per gene in order to remove duplicate probes.

Weighted gene co-expression network analysis (WGCNA) was performed as follows.

Log 2 normalized microarray expression values were used as input to WGCNA (v1.60) to conduct an unsupervised clustering analysis, resulting in co-expression modules (groups of densely interconnected genes) which correspond to comparably regulated biological pathways. For each experiment, an approximately scale-free topology matrix (TOM) was first calculated to encode the network strength between probes. Probes were clustered into WGCNA modules based on TOM distances. Resultant dendrograms of correlation networks were trimmed to isolate individual modular groups of probes, labeled using semi-random color assignments, based on a detection cut height of 1 and a merging cut height of 0.2, with the additional use of a partitioning around medoids function. Final membership of probes representing the same gene into modules was based on selection of the greatest within-module correlation with module eigengene (ME) values. Expression profiles of genes within modules were summarized by the ME, the module's first principal component. MEs act as characteristic expression values for their respective modules and can be correlated with sample traits such as cell type, cohort (healthy control or SLE), or serological measurements. This was performed using Pearson correlation for continuous traits and using point-biserial correlation for dichotomous traits. The correlation coefficient of each gene in a module with the module eigengene (kME), a metric for module membership, was used to determine the association of individual genes with the expression of the module as a whole. The mean kME of all genes in a module was taken as a metric of overall module quality. If the genes in a module have low kMEs, it may be indicative that a few highly variable genes have dominated the eigengene calculation. Modules with mean kMEs close to 1 were considered to be high-quality, and modules with mean kMEs close to zero were considered to be low-quality. When analyzing multiple data sets, the grand mean was the mean of the mean kMEs for each data set.

Functional gene characterization and pathway identification were performed as follows. The Biologically Informed Gene Clustering (BIG-C) tool was used to characterize genes into functional groups utilizing publicly available information from online tools and databases including UniProtKB/Swiss-Prot, GO Terms, KEGG pathways, NCBI PubMed, and the Interactome. DE genes were assigned into functional groups using BIG-C and signaling molecules and transcription factors upstream of DE genes were identified using IPA Upstream Regulator (UR) analysis. For each regulator, an activation z-score was calculated strictly from experimentally observed information provided for the downstream targets, and an overlap p-value was calculated through Fischer's exact test.

Gene set variation analysis (GSVA) was performed as follows. GSVA (V1.25.4) software package for R/Bioconductor was used as a non-parametric, unsupervised method for estimating the variation of pre-defined gene sets in patient and control samples of microarray expression data sets. The input for the GSVA algorithm was a gene expression matrix of log 2 microarray expression values and a collection of pre-defined gene sets or database of pre-exiting gene sets (MSig). Enrichment scores (GSVA scores) were calculated non-parametrically using a Kolmogorov Smirnoff (KS)-like random walk statistic and a negative value for a particular sample and gene set. Significance of functional enrichment was calculated using a chi-squared test and categories with p-values less than 0.05 were considered significantly enriched.

Network analysis and visualization were performed as follows. Visualization of protein-protein interactions and relationships between genes within datasets was done using the Cytoscape (V3.6.0) software and the stringApp (V1.3.2) plugin application. The Clustermaker2 App (V1.2.1) plugin was used to create clusters of the most related genes within a dataset using a network scoring degree cutoff of 2 and setting a node score cut-off of 0.2, a k-Core of 2, and a max depth of 100.

CIRCOS visualization was performed as follows. CIRCOS (V0.69.3) software was used to visualize datasets.

Drug target prediction was performed as follows. Queries of the perturbation database from the Broad Institute Library of Integrated Network-Based Cellular Signatures (LINCS) were utilized to predict potentially useful therapeutic compounds and to confirm the dysregulation of upstream target genes in SLE patient MC by assessing signatures of significantly up- and down-regulated genes for input to the lincscloud API (available at data.lincscloud.org.s3.amazonaws.com/index.html). The LINCS L1000 platform was developed using Luminex Flexmap 3D bead technology that contained far greater probe sets than the hgU133 arrays. The LINCS L1000 contains representative information relating expression of 978 “landmark genes” that was generated from 25 cell types that were antagonized by drugs and gene over-expression or silencing interventions.

Prediction of disease activity from WGCNA module enrichment was performed using machine learning as follows. 4 whole blood (WB) and 2 peripheral blood mononuclear cell (PBMC) microarray datasets containing gene expression data from lupus patients were obtained from the GEO repository or from collaborators (GSE45291, GSE39088, GSE49454, GSE72747, GSE50772, FDAPBMC3). Raw data was curated and normalized. In addition, low-intensity probes were filtered, and duplicate probes mapping to the same gene symbol were filtered based on interquartile range. Datasets were batch corrected to account for platform differences using the ComBat R package and merged by matching gene symbols. WGCNA was applied to CD4 T cells (GSE10325), CD19 B cells (GSE10325), CD33 MC (GSE10325), CD14 MC (GSE38351), and low-density granulocytes (LDG) (GSE26975) to acquire gene modules with significant correlations with or against SLEDAI. GSVA was used to test the merged blood dataset for the presence of these modules as well as lists of genes positively and negatively associated with lupus plasma cells (PC). GSVA scores were used as input to a generalized linear model (GLM) from the glmnet R package to predict disease activity, and receiver operating characteristic (ROC) curves were generated using the pROC R package. Patient-by-patient enrichment of cell types was assessed based on the expected versus observed enrichment of each WGCNA module. Odds ratios (OR) for active disease were calculated according to the following formula:

$$\frac{( enriched active ) * ( non \text{-} enriched inactive )}{( non \text{-} enriched active ) * ( enriched inactive )}$$

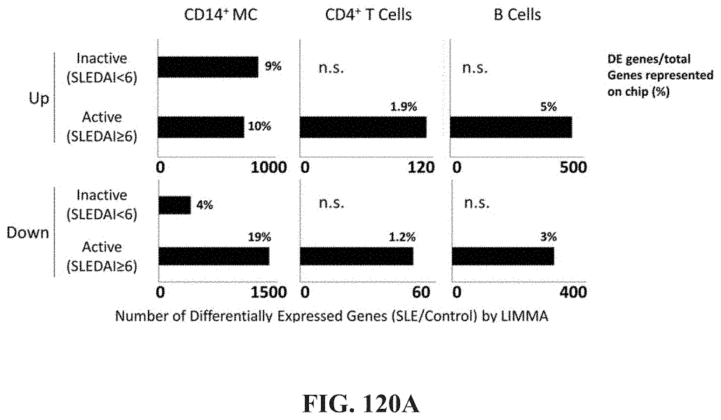

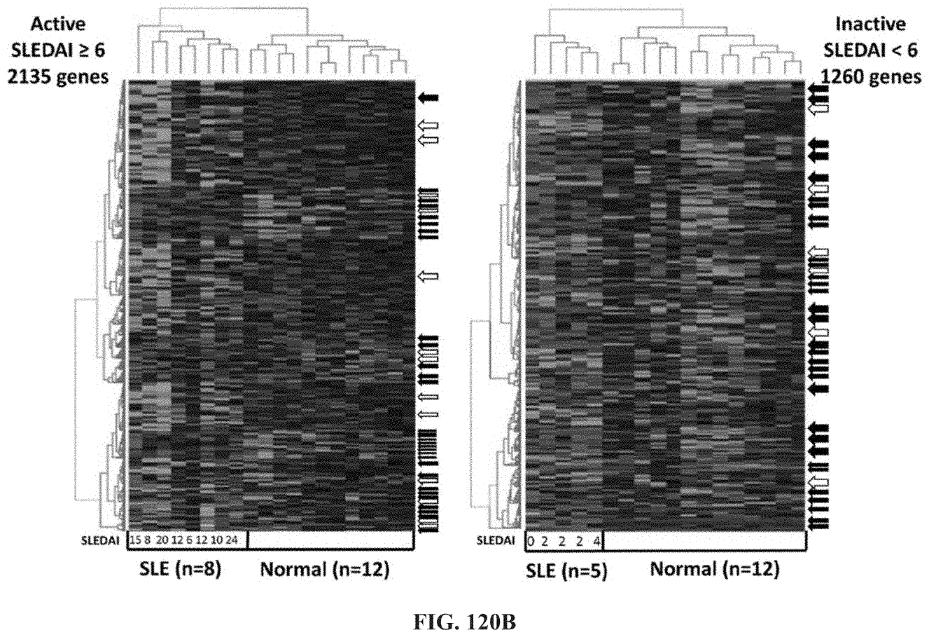

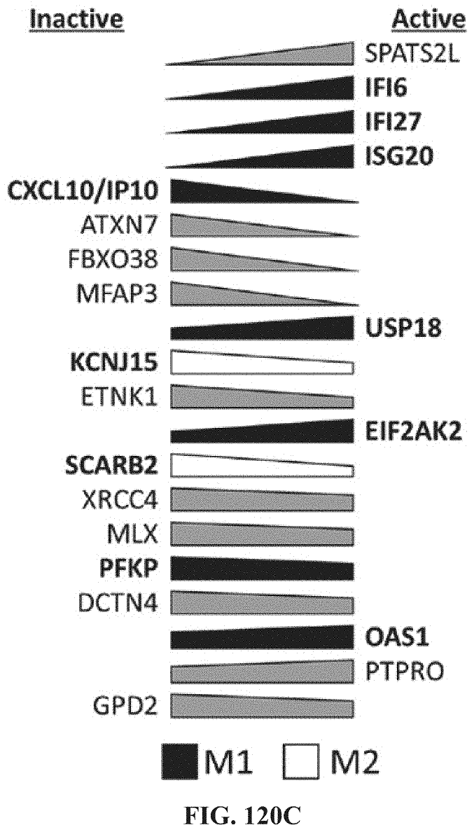

Differential expression of MC genes in active and inactive SLE was analyzed as follows. To assess the contribution of MC to SLE pathogenesis, gene expression profiles of CD14+ MC from SLE patients with varying levels of disease severity were analyzed. In order to compare the role of MC in SLE to that of B and T cells, a consensus DE gene signature was generated for each (GSE10325 and GSE51997 for CD19+ B cells, CD10325 and CD4588 for CD4+ T cells). Large numbers of DE genes were found in MC from both active (2,135) and inactive (1,260) SLE patients (FIG. 120A). In contrast, hundreds of statistically significant (FDR less than 0.2) DE genes were identified in B and CD4 T cells of active SLE patients (760 and 164 genes, respectively), whereas there were no significant DE genes found in B or CD4 T cells from patients with inactive SLE compared to healthy controls (FIG. 120A).

FIG. 120A shows the number of differentially expressed (DE) genes detected by LIMMA analysis in MC, CD4+ T cells, and B cells isolated from inactive (SLEDAI<6) and active (SLEDAI≥6) SLE patients when compared to healthy donors. n.s.: no genes found to be significantly differentially expressed (FDR<0.2) when compared to healthy controls. FIG. 120B shows Hierarchical clustering of differentially expressed (DE) genes detected by LIMMA analysis in CD14+ MC isolated from inactive (SLEDAI<6) and active (SLEDAI≥6) SLE patients when compared to healthy donors. Arrows highlight M1 (black) or M2 (white) polarization genes. FIG. 120C shows fold change variation of genes found to be upregulated in both active and inactive SLE MC. Polarization-related genes are shown in bold and M1 genes are represented by a black wedge while M2 genes are represented with a white wedge. Genes not associated with M1 or M2 pathways are represented with a gray wedge.

Hierarchical clustering of DE genes in CD14+ MC isolated from inactive and active SLE patients when compared to healthy donors cleanly sorted patient samples by disease cohort (FIG. 120B). Although they did not tend to group into discrete clusters, several genes involved in Mϕ activation were observed among the DE genes in both active and inactive patients. Cross-referencing with a list of experimentally determined human Mϕ differentiation and activation genes revealed alterations in the Mϕ activation signature between active and inactive SLE patients: M1-associated genes tended to be upregulated in both active and inactive SLE compared to healthy donors (94% and 97%), while M2-associated genes tended to be more upregulated in inactive SLE patients (86%) than active SLE patients (38%) compared to healthy donors (FIG. 120B). As Mϕ activation may encompass a spectrum of functional phenotypes controlled by finely-tuned molecular rheostats, the fold change of DE genes that were commonly upregulated in CD14+ MC were compared from both active and inactive SLE patients; this demonstrated that common M1-associated genes (black wedges) were more highly upregulated in active patients, whereas common M2-associated genes (white wedges) were more highly upregulated in inactive patients (FIG. 120C). A few of these commonly upregulated genes were not associated with either M1 or M2 pathways (gray wedges).

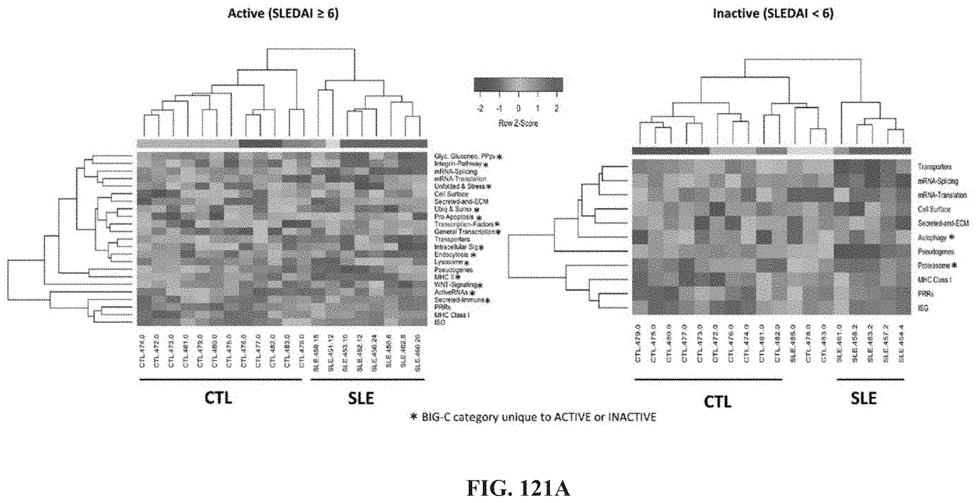

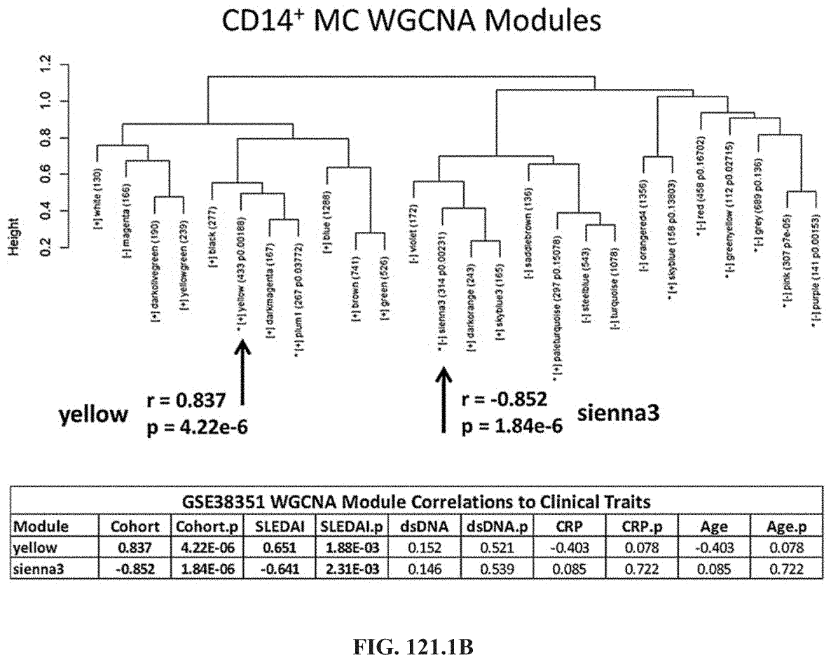

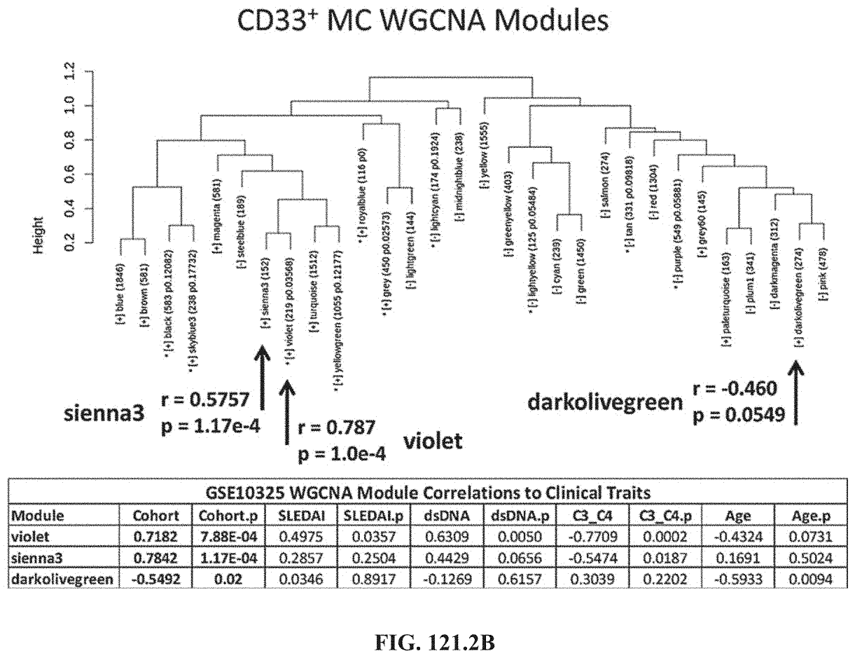

Functional characterization of DE gene signatures in CD14+ MC isolated from SLE patients was performed as follows. Next, the potential functional changes represented by the divergent activation signatures in SLE MC were characterized. Biologically Informed Gene Clustering (BIG-C) is a functional aggregation tool developed to understand the biological groupings of large lists of genes. Genes are sorted into 54 categories based on their most likely biological function and/or cellular localization determined from information from multiple online tools and databases. The DE genes from active and inactive CD14+ MC were analyzed by Gene Set Variation Analysis (GSVA) to determine enrichment of BIG-C functional categories. The active and inactive CD14+ MC samples shared a common BIG-C profile generally related to IFN signaling and inflammation, including the MHC class I/II, ISG, immune secreted, transcription, endosomal recycling, immune signaling, and TLR & DAMP categories (FIG. 121A). Interestingly, BIG-C categories unique to each cohort (starred) confirm effector function upregulation in MC derived from active SLE (biochem, chromatin, anti-apoptosis down; activeRNAs, secreted & extracellular matrix, immune cell surface, vesicles & endosome up) and a preference for catabolic pathways in MC derived from inactive SLE (cell surface, DNA repair down; ubiquitylation and sumoylation up) (FIG. 121A). Additionally, unique enrichment of the MT OX PHOS pathway in MC from inactive SLE minors findings that pro-resolving M2 Mϕ predominantly obtain energy from oxidative metabolism.

FIG. 121A shows DE genes from active and inactive CD14+ MC were analyzed by GSVA to determine pathway enrichment using functional definitions provided from the BIG-C (Biologically Informed Gene Clustering) annotation library. Samples were successfully sorted by disease cohort via this method in both active and inactive MC. Starred BIG-C categories only appeared in the active or inactive analysis, respectively. FIG. 121B shows WGCNA of CD14+ and CD33+ MC isolated from SLE patients. Dendrograms show hierarchy of modules formed by unsupervised WGCNA clustering of DE genes from CD14+ and CD33+ MC isolated from active and inactive SLE patients.

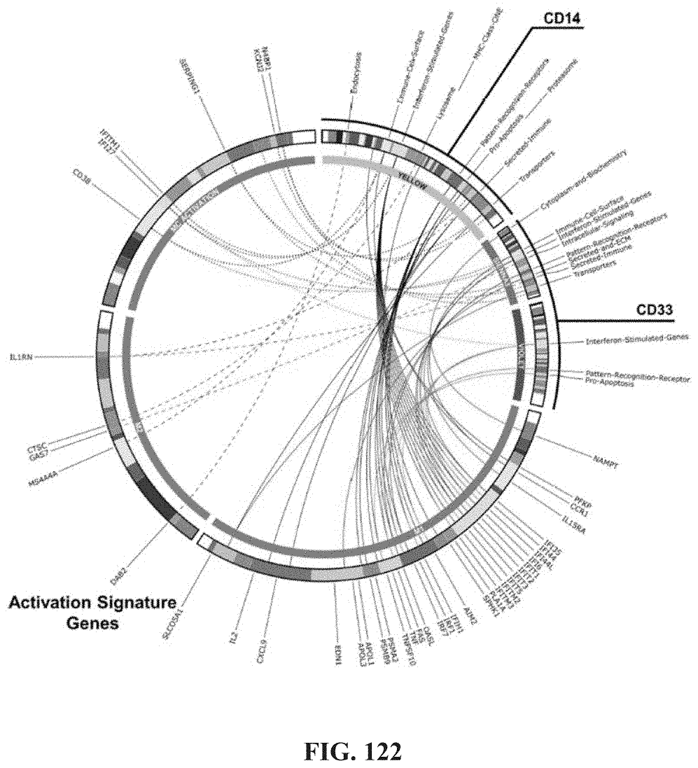

MC activation signature genes found in disease-correlated WGCNA modules from active SLE MC were analyzed as follows. In order to determine the gene signatures that were relevant to SLE pathogenesis in an unbiased manner, gene expression modules were generated via WGCNA with correlation to clinical traits, and then prioritized with correlation to disease cohort and even eigengene distribution to exclude modules whose assembly were driven primarily by a single eigengene. As the CD33+ dataset contained no inactive SLE patients, data from only active SLE patients was used to construct modules for comparison. The CD14+ dataset produced one module with significantly positive correlation to SLE (yellow: n=362, r=0.837, p=4.22e-6) and one module with significantly negative correlation to SLE (sienna3: n=229, r=−0.852, p=1.84e-6), and the CD33+ dataset produced two modules significantly positively correlated to SLE (violet: n=182, r=0.718, p=7.88e-4; sienna3: n=133, r=0.784, p=1.17e-4) and one module significantly negatively correlated to SLE (darkolivegreen: n=227, r=−0.549, p=0.0182) (FIG. 121B). Notably, the CD14+-derived modules were also significantly correlated to SLEDAI (r=0.651, p=1.88e-3 and r=−0.641, p=2.31e-3 respectively). The significantly positive disease-correlated modules from the CD14+ dataset contained several activation-related genes, mostly concentrated in the apoptosis, ISG, and PRR BIG-C categories (visualized in FIG. 122). While the yellow module was heavily enriched for M1-related genes, four M2-related genes were also present. Of the 37 genes in this module that were associated with MC activation, 27 (73%) were M1-related genes. The CD33+ modules each contained far fewer activation genes and almost no M2 signature. Despite this, of the 29 MC activation-associated genes in both these modules combined, 21 (72%) were M1 genes. The CD14+ negatively-correlated module (sienna3) contained no MC activation genes and the CD33+ negatively-correlated module (darkolivegreen) contained only one, GAS7. These findings indicate that Mϕ activation state contributes heavily to the differential MC DE gene signature between active and inactive SLE. Furthermore, the polarization genes present are nearly exclusively M1-associated, demonstrating that the observed differences in Mϕ polarization may be driving enhanced inflammation in active SLE.

FIG. 122 shows a CIRCOS diagram comparing the composition of SLE positively-correlated CD14+ and CD33+ WGCNA modules to genes enriched in M1- or M2-polarized human Mϕ or genes associated with general MC activation (upregulated in both M1 and M2 conditions). Genes found in the yellow module (CD14+) are shown in black, genes found in the violet module (CD33+) are shown in red, and genes found in the sienna3 module (CD33+) are shown in orange. M1-related genes are represented with solid lines, M2-related genes are represented by dashed lines, and general MC activation genes are represented with dotted lines.

Protein interaction-based clustering of genes in WGCNA modules significantly correlated to disease activity was performed as follows. Next, a more detailed analysis of the composition of the WGCNA modules significantly correlated to disease activity was performed by using Cytoscape with the stringApp and MCODE plugins to create protein-protein interaction networks and clusters. The resulting networks were further simplified into metastructures defined by the number of genes in each cluster, the number of significant intra-cluster connections identified by MCODE, and the strength of associations connecting members of different clusters to each other. This dual approach allowed a comparison of the overall topology of different WGCNA clusters while also noting genes of interest and their groupings.

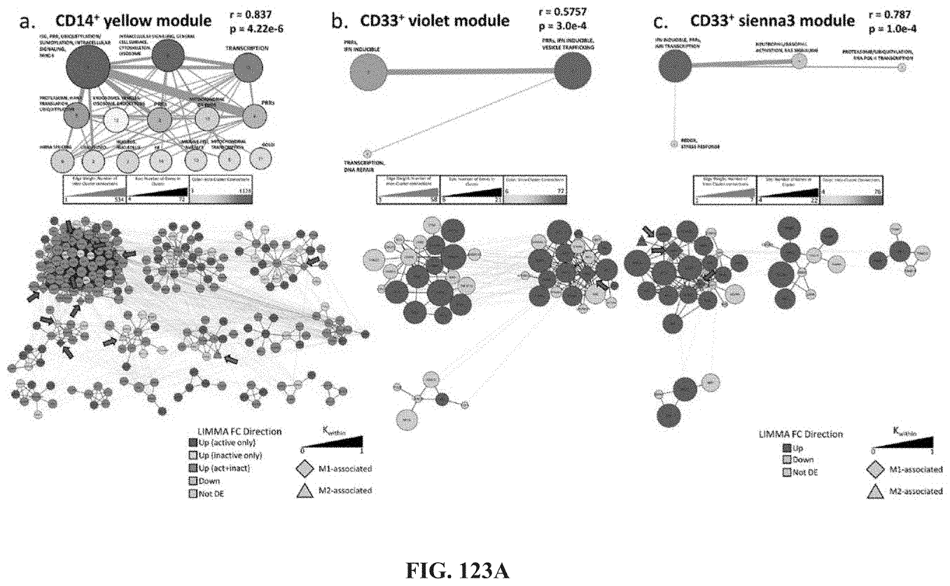

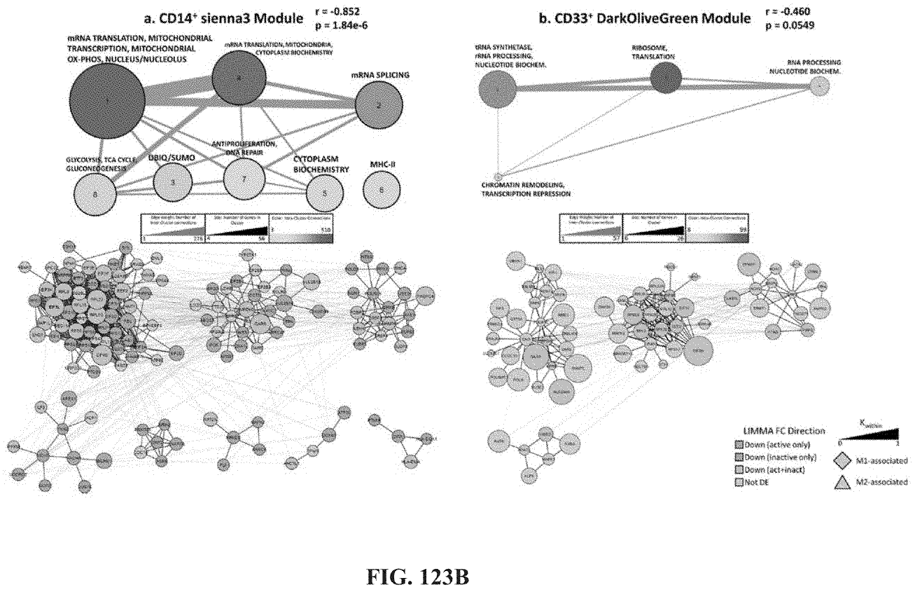

The largest and most internally connected cluster of genes in the CD14+ yellow module (positively correlated to disease activity, FIG. 121B) was dominated by ISG and PRR-related genes and contained several members of the ubiquitin C pathway, a gene network not present in either of the positively correlated CD33+ modules (FIG. 123A, top). Interestingly, further analysis of this cluster and the closely related proteasome/mRNA translation/ubiquitylation cluster revealed several upregulated activation-induced genes, including M1-associated genes (FIG. 123A(a), bottom, red arrows). Two of the four M2-associated genes in the module (CTSC and IL1RN) appeared in smaller PRR and vesicle-associated clusters (FIG. 123A(a), blue arrows). Similar PRR/vesicle clusters were found in the two positively correlated (FIG. 121B) CD33+ modules, but only three M1 genes appeared in these clusters (FIGS. 123A(b) and 123A(c); red arrows). Taken together, these data demonstrate that dysregulated activation signals in CD14+ MC drive SLE pathogenesis, especially in patients with active disease. The two WGCNA modules negatively correlated to SLEDAI (FIG. 2B, sienna3 for CD14+ and darkolivegreen for CD33+) were less informative and broadly mirrored each other in content, both containing networks related to RNA synthesis and processing, translation, and DNA maintenance (FIG. 4B). Two clusters that arose from the CD14+ module represented pathways not present in the CD33+ module: glycolysis/TCA cycle/gluconeogenesis in cluster 8 and ubiquitylation/sumoylation in cluster 3. The majority of the genes in these clusters were selectively downregulated in active SLE only (FIG. 123B(a)).

FIGS. 123A-123B show protein-protein interaction networks and clusters generated via CytoScape using the STRING and MCODE plugins. Networks were constructed of the gene lists of WGCNA modules positively (FIG. 123A, above) or negatively (FIG. 123B, below) correlated to SLEDAI from CD14⁺ MC (FIG. 123A(a) and FIG. 123B(a)) or CD33⁺ MC (FIG. 123A(b), FIG. 123A(c), FIG. 123B(b), and FIG. 123B(c)). MCODE clusters are determined by the strength of protein-protein interactions, calculated by pooling information from publicly available literature. Top half of diagrams show the cluster metastructure of each network while bottom half shows the specific genes that make up each cluster. M1-related genes are indicated by red arrows and M2-related genes are indicated by blue arrows.

Predicted compounds targeting CD14+ MC pathways in SLE were analyzed as follows. With the goal of identifying novel potential therapies for SLE, DE gene data from CD14+ MC were used as input for LINCS, a drug discovery tool based upon gene expression changes induced by perturbagens in a variety of reference cell lines. The result is a list of drugs that counteract the genomic changes that propagate disease, determined in an unbiased manner and based on empirical data.

Summarized results of the LINCS analysis are presented in Table 68 and Table 69 for the CD14+ MC obtained from active SLE patients and inactive SLE patients, respectively. Compounds directed against a shared target are collapsed into each category, allowing calculation of LINCS connectivity score statistics for all drugs impacting that target. The drug with the strongest connectivity score for each target is shown in the “Representative Drug” column. Notably, 49% of targets and 44% of representative drugs were suggested by LINCS for both active and inactive SLE MC (Table 68 and Table 69, bolded). The results were cross-referenced against FDA and clinical trial databases, revealing that many of the LINCS-suggested drugs are either already approved or in trials for non-lupus indications, underscoring their potential for swift and successful drug repositioning (Table 68 and Table 69, indicated by †; and ‡).

TABLE 68
Compounds targeting CD14+ monocyte pathways in active SLE

| Target | Count | Range | Mean ± SEM | Representative Drug |
|---|---|---|---|---|
| Farnesyl transferase | 2 | (−95.98)-(−99.61) | −97.79 ± 1.81 | Tipifarnib‡ |
| Acetylcholinesterase | 2 | (−93.16)-(−98.09) | −95.63 ± 2.47 | Mestinon† |
| PKC (pan) | 2 | (−93.81)-(−97.19) | −95.50 ± 1.69 | bisindolylmaleimide-ix |
| mTORC1/2 (Tacrolimus⁵†⁾ | 6 | (−89.05)-(−99.66) | −94.70 ± 1.5 | KU-0063794 |
| Sigma Receptor | 2 | (−86.80)-(−94.33) | −90.56 ± 3.76 | BD-1063 |
| P13K (pan) (Idealalisib¹†⁾ | 2 | (−86.90)-(−93.15) | −90.02 ± 3.12 | GSK-1059615 |
| ROCK-1/2 (KD025⁷‡⁾ | 3 | (−79.19)-(−95.90) | −89.96 ± 5.39 | GSK-429286A |
| PLK1 | 2 | (−87.31)-(−92.57) | −89.94 ± 2.63 | ON-01910 |
| IGF-1R | 5 | (−76.11)-(−99.20) | −89.87 ± 4.24 | GSK-1904529A |
| mTORCI (Tacrolimus⁵†⁾ | 3 | (−85.83)-(−97.22) | −89.83 ± 3.7 | AZD-8055 |
| HDM2 | 3 | (−81.37)-(−96.81) | −89.73 ± 4.5 | HLI-373 |
| Ca channel | 9 | (−82.26)-(−99.98) | −89.70 ± 2.45 | Nifedipine† |
| GR agonist | 12 | (−74.94)-(−99.03) | −89.61 ± 2.18 | Dexamethasone†^{T} |
| CDK1, 2, 5 (palbociclid⁴†⁾ | 2 | (−88.47)-(−89.62) | −89.05 ± 0.58 | Aloisine |
| PI3Kg | 2 | (−81.84)-(−96.21) | −89.03 ± 7.19 | AS-605240 |
| DNA-PK | 2 | (−88.33)-(−88.67) | −88.50 ± 0.17 | NU-7026 |
| MAP2K1/2 | 6 | (−78.58)-(−97.06) | −88.42 ± 2.9 | U0126 |
| MAPK | 5 | (−81.41)-(−92.96) | −88.39 ± 2.18 | EO-1428 |
| Tyrosine Kinase (board) | 4 | (−82.14)-(−98.80) | −88.30 ± 3.83 | Lestaurtinib‡ |
| PARP-1 (Niraparib³†⁾ | 5 | (−79.37)-(−91.68) | −87.95 ± 2.38 | Rucaparib‡ |
| PDGFR | 2 | (−87.63)-(−88.20) | −87.91 ± 0.28 | tyrphostin-AG- 1295 |
| JKN (pan) | 2 | (−82.84)-(−92.78) | −87.81 ± 4.97 | AS-601245 |
| EGFR (Gefitinib¹†⁾ | 10 | (−74.73)-(−99.36) | −87.27 ± 2.99 | Lapatinib⁰† |
| b2 adrenergic receptor agonist | 6 | (−80.38)-(−93.93) | −87.27 ± 2.23 | Formotcrol† |
| 5-HT 1B agonist | 3 | (−83.27)-(−89.31) | −86.94 ± 1.86 | Anpirtoline |
| topoisomerase I (Irinotecan⁻¹†⁾ | 2 | (−82.61)-(−90.87) | −86.74 ± 4.13 | Topotecan† |
| topoisomerase II | 3 | (−81.58)-(−90.76) | −86.55 ± 2.68 | Rozoxane† |
| Proton pump | 2 | (−85.41)-(−87.66) | −86.53 ± 1.13 | Rabeprazole† |
| NMPRTase | 3 | (−77.90)-(−94.32) | −85.94 ± 4.74 | APO-866 |
| Enkephalinase | 2 | (−84.66)-(−86.33) | −85.49 ± 0.83 | Thiorphan‡ |
| Angiotensin II receptor | 2 | (−84.6.)-(−85.32) | −84.96 ± 0.36 | Telmisartan† |
| Aurora kinase A | 2 | (−84.83)-(−84.87) | −84.85 ± 0.02 | MLN-8054‡ |
| PI3Kb | 3 | (−80.29)-(−88.89) | −84.77 ± 2.49 | TGX-221 |
| K channel | 3 | (−78.57)-(−87.81) | −84.42 ± 2.94 | Pazilline |
| B3 adrenergic receptor agonist | 3 | (−77.43)-(−91.37) | −83.95 ± 4.05 | L-755507 |
| PDE4 (Roflumilast⁶†⁾ | 2 | (−79.38)-(−87.35) | −83.36 ± 3.98 | Ibudilast‡ |
| HMG-Co A reductase (Statins³†⁾ | 6 | (−76.34)-(−95.08) | −83.19 ± 3.05 | Atorvastatin†^{T} |
| ER (pan) (Tamoxifen²†⁾ | 3 | (−75.46)-(−87.80) | −82.61 ± 3.70 | Clomifene‡ |
| VEGFR2 (Sorafenib⁻³‡⁾ | 2 | (−75.75)-(−86.67) | −81.21 ± 5.46 | Orantinib‡ |
| Na channel | 2 | (−75.75)-(−85.04) | −80.39 ± 4.64 | Benzamil |
| ATM Kinase | 2 | (−78.69)-(−80.42) | −79.55 ± 0.87 | CP466722 |
| AMPA receptor | 2 | (−77.82)-(−80.21) | −79.02 ± 1.20 | GYKI-52466 |
| Wnt (pan) | 2 | (−76.35)-(−80.74) | −78.55 ± 2.20 | PNU-74654 |
| HSP90 | 2 | (−76.64)-(−79.99) | −78.32 ± 1.68 | Gedunin |
| SERT | 2 | (−75.00)-(−75.31) | −75.16 ± 0.15 | Duloxetine† |

TABLE 69
Compounds targeting CD14+ monocyte pathways in inactive SLE

| Target | Count | Range | Mean ± SEM | Top Drug |
|---|---|---|---|---|
| PKC (pan) | 2 | (−98.19)-(−99.03) | −97.79 ± 1.81 | Bisindoylmalcimide-ix |
| IGF-1R | 4 | (−93.01)-(−98.13) | −96.56 ± 1.21 | BMS-536924 |
| mTORC1/2 | 6 | (−90.39)-(−99.97) | −96.00 ± 1.72 | KU-0063794 |
| (Tacrolimus⁵†) | | | | |
| Farnesyl transferase | 2 | (−94.89)-(−96.95) | −95.92 ± 1.03 | Tipifarnib‡ |
| PI3K (pan) | 2 | (−90.28)-(−99.74) | −95.01 ± 4.73 | GSK-1059615 |
| (Idelalisib¹†) | | | | |
| Topoisomerase I | 3 | (−90.33)-(−97.63) | −94.70 ± 2.23 | Topotecan† |
| (Irinotecan⁻¹†) | | | | |
| mTORC1 | 4 | (−86.57)-(−98.97) | −94.29 ± 2.87 | AZD-8055 |
| (Tacrolimus⁵†) | | | | |
| HDM2 | 3 | (−87.19)-(−96.91) | −93.25 ± 3.05 | JNJ-26854165 |
| B-Raf | 2 | (−89.18)-(−97.19) | −93.19 ± 4.01 | Vemerafenib⁻⁶† |
| FAAH | 2 | (−90.89)-(−95.32) | −93.10 ± 2.22 | PF-3845 |
| ROCK-1/2 | 2 | (−91.66)-(−93.36) | −92.51 ± 0.85 | Y-27632 |
| (KD025⁷‡) | | | | |
| PI3Kb | 3 | (−82.45)-(−98.27) | −92.34 ± 4.98 | PI-828 |
| (Idelalisib¹†) | | | | |
| MAP2K1/2 | 6 | (−87.89)-(−98.76) | −92.17 ± 1.57 | U0126 |
| DNA-PK | 2 | (−85.41)-(−97.43) | −91.42 ± 6.01 | NU-7026 |
| PI3Kg | 2 | (−87.49)-(−94.95) | −91.22 ± 3.73 | AS-605240 |
| TRPV1 | 2 | (−88.65)-(−92.83) | −90.74 ± 2.09 | Eriodictyol |
| COX-1 | 2 | (−88.36)-(−92.43) | −90.40 ± 2.03 | Eicosatctraynoic-acid |
| PARP-1 | 2 | (−89.49)-(−91.14) | −90.31 ± 0.83 | Olaparib⁰† |
| (niraparib³†) | | | | |
| HMG-CoA reductase | 7 | (−79.06)-(−97.06) | −89.27 ± 2.37 | Atorvastatin†^{T} |
| (Statins³†) | | | | |
| NKI | 2 | (−79.34)-(−97.98) | −88.66 ± 9.32 | FK-888 |
| Syk | 2 | (−81.58)-(−94.43) | −88.01 ± 6.43 | Fostamatinib⁷‡ |
| 5-HT 1B agonist | 2 | (−85.72)-(−90.07) | −87.90 ± 2.18 | 5-nonyloxytryptamine |
| NMPRTase | 2 | (−82.20)-(−92.79) | −87.50 ± 5.30 | CAY-10618 |
| Sigma receptor | 2 | (−86.24)-(−88.50) | −87.37 ± 1013 | BD-1063 |
| Ca channel | 11 | (−76.85)-(−99.83) | −87.01 ± 2.48 | Nifedipine† |
| Adrenergic receptor | 2 | (−76.15)-(−97.26) | −86.71 ± 10.56 | dopamine† |
| (pan) agonist | | | | |
| CB2 agonist | 3 | (−82.69)-(−92.44) | −86.51 ± 3.01 | GW-405833 |
| SERT | 4 | (−76.46)-(−93.45) | −85.91 ± 4.03 | Paroxetine† |
| Topoisomerase II | 5 | (−77.70)-(−94.56) | −85.78 ± 2.90 | Razoxane† |
| EGFR | 16 | (−74.54)-(−99.27) | −85.32 ± 2.00 | Lapatinib⁰† |
| (Gefitinib¹†) | | | | |
| Tyrosine kinase | 4 | (−79.11)-(−99.44) | −85.16 ± 4.79 | lestaurtinib‡ |
| (broad) | | | | |
| MAPK | 2 | (−74.80)-(−95.28) | −85.04 ± 10.24 | JX-401 |
| 5-HT 4 | 2 | (−81.68)-(−87.92) | −84.80 ± 3.12 | RS-23597-190 |
| ER (pan) | 6 | (−75.38)-(−96.20) | −84.18 ± 2.99 | Clomifene‡ |
| VEGFR (pan) | 3 | (−77.91)-(−87.26) | −83.40 ± 2.82 | Tivozanib‡ |
| (sorafenib⁻³‡) | | | | |
| BCL-2 | 2 | (−77.03)-(−89.71) | −83.37 ± 6.34 | ABT-737‡ |
| (Venetoclax⁰†) | | | | |
| ATM Kinase | 2 | (−77.51)-(−89.82) | −82.67 ± 7.15 | KU-55933 |
| c-Met | 2 | (−77.20)-(−88.11) | −82.65 ± 5.46 | SU-11274 |
| Proton pump | 2 | (−78.63)-(−84.47) | −81.55 ± 2.92 | Rabeprazole† |
| GR agonist | 3 | (−75.23)-(−89.53) | −81.19 ± 4.30 | Dexamethasone†^{T} |
| H3 receptor | 2 | (−76.33)-(−85.49) | −80.91 ± 4.58 | iodophenpropit |
| Auroa kinase A | 2 | (−78.64)-(−81.87) | −80.26 ± 1.61 | MLN-8054‡ |
| B3 adrenergic | 2 | (−77.27)-(−82.22) | −79.74 ± 2.47 | SR-59230A |
| receptor agonist | | | | |
| VEGFR2 | 2 | (−76.31)-(−81.30) | −78.81 ± 2.49 | SU-4312 |
| (Sorafenib⁻³‡) | | | | |
| B2 adrenergic | 3 | (−77.03)-(−78.85) | −78.00 ± 0.53 | Fenoterol‡ |
| receptor agonist | | | | |
| Na channel | 2 | (−76.38)-(79.17) | −77.78 ± 1.40 | benzamil |

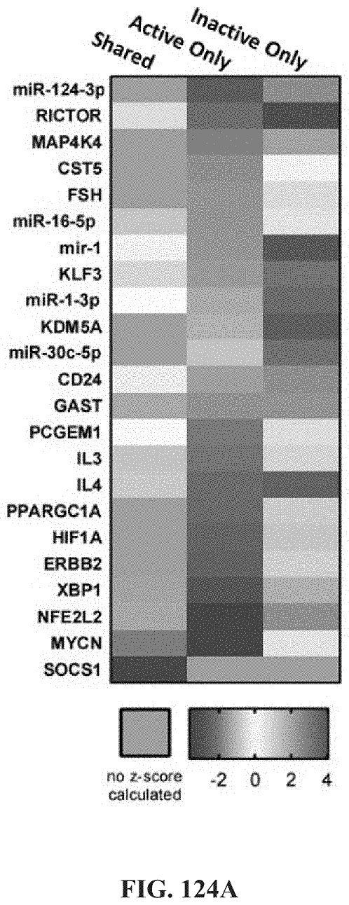

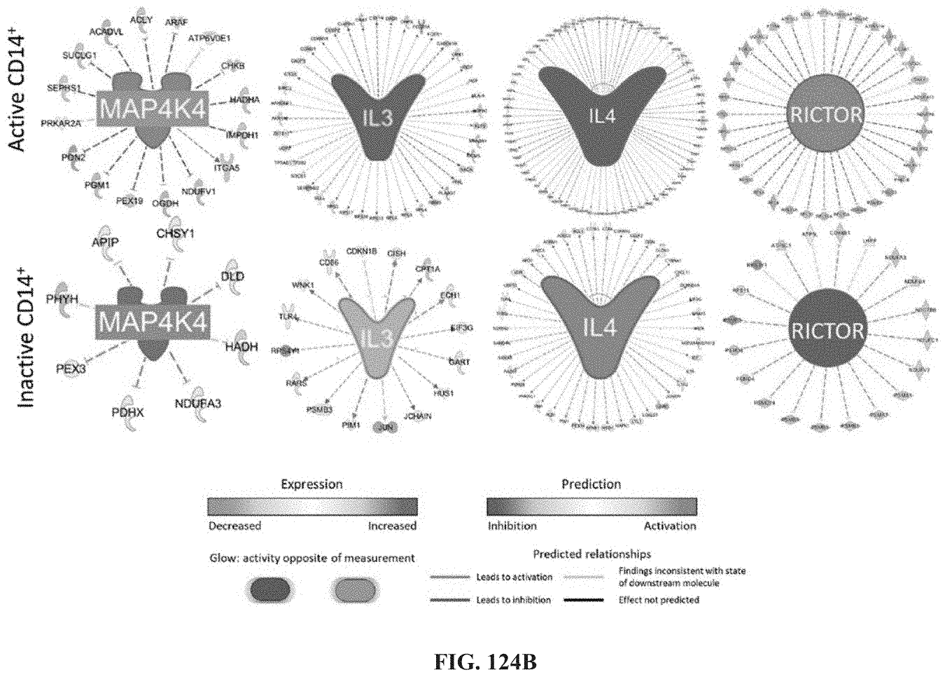

Projected upstream regulator genes in CD14+ MC isolated from active and inactive SLE patients were analyzed as follows. To investigate the intracellular signaling pathways at play, IPA was employed to analyze the CD14+ MC DE dataset and identify potential biologic upstream regulators (BURs) for MC from active patients, inactive patients, and the active-inactive overlap (FIG. 124A). Genes for which IPA indicated a z-score of at least 2 in at least one of the three sets are shown. Several of the resulting genes may be major regulators of MC polarization, including the M1 regulators MAP4K4 and mir-1 and the M2 regulators IL3, IL4, PPARGC1A, HIF1A, and NFE2L2 (FIG. 5A). Notably, the z-scores show a clear delineation of their opposing activities in active SLE patient MC vs. inactive SLE patient MC, with M1 regulators displaying positive z-scores in active patients and negative z-scores in inactive patients and vice-versa for M2 regulators. Each of these trends was supported by the corresponding expression of several downstream genes known to interact with each upstream regulator (FIG. 124B). Interestingly, only one gene that may be involved in Mϕ polarization had a z-score that contradicted this pattern: RICTOR, a relative of mTOR and a subunit of the mTORC2 complex, may be shown to suppress M1 polarization in mice yet is identified by IPA as an upstream regulator of CD14+ MC from active SLE patients.

FIG. 124A shows that IPA was used to analyze the CD14⁺ MC dataset and identify putative upstream regulators for active patient monocytes, inactive patient monocytes, and the active-inactive overlap using a p-value cutoff of 0.05. Only genes for which IPA assigned a z-score of ≥|2| in at least one of the three sets are shown. FIG. 124B shows representative diagrams showing downstream gene expression changes (outer circles) used to calculate upstream regulators (center).

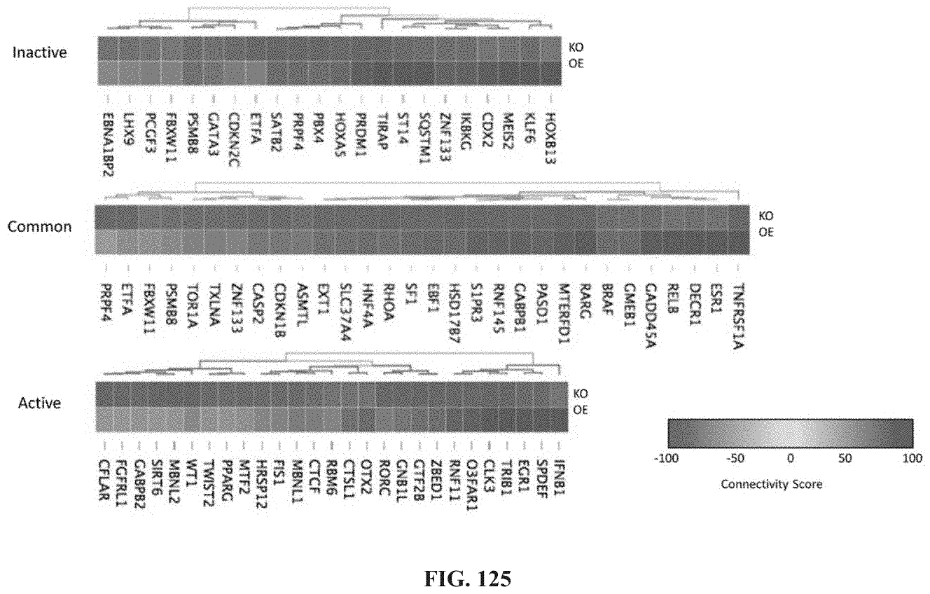

Also, the gene connectivity scores from the collection of knockdown and overexpression experiments present in the LINCS database were used to identify BURs determined primarily by empirical results. Genes were identified as BURs for a particular dataset if they received a knockdown connectivity score between −75 and −100 and an overexpression connectivity score between 50 and 100 for that dataset. This approach produced 17 BURs unique to the inactive SLE cohort, 31 BURs unique to the active SLE cohort, and 30 BURs common to both (FIG. 125). These regulators were distinct from those identified by IPA, representing additional potential drug targets.

FIG. 125 shows gene sets from CD14⁺ MC isolated from active or inactive SLE patients were used as input for the LINCS analysis platform, which reports connectivity scores for individual genes that describe how well the genomic change between the baseline and experimental input sets matches the change observed following the knockdown or overexpression of the individual gene in question. Knockdown and overexpression data were filtered by genes for which LINCS reported connectivity scores for both categories, and genes were identified as BURs for a particular dataset if they received a knockdown connectivity score between −75 and −100 and an overexpression connectivity score between 50 and 100 for that dataset.

Machine learning results confirmed that gene modules from MC predict SLE activity in unrelated data sets. The relationships between MC gene expression and SLE activity indicated that a machine learning method may be able to predict disease activity when “trained” with MC gene signatures. Toward this end, unrelated WB and PBMC datasets were merged into a test set and analyzed for MC WGCNA module enrichment via GSVA. In order to compare the predictive power of MC gene signatures, WGCNA modules were also generated for CD4 T cells, CD19 B cells, plasma cells (PC) and low-density granulocytes (LDG) and employed in a similar manner to predict disease activity.

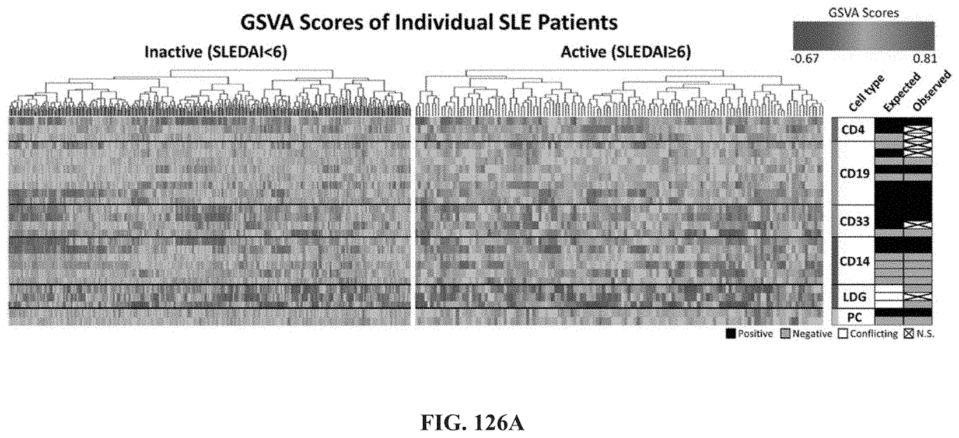

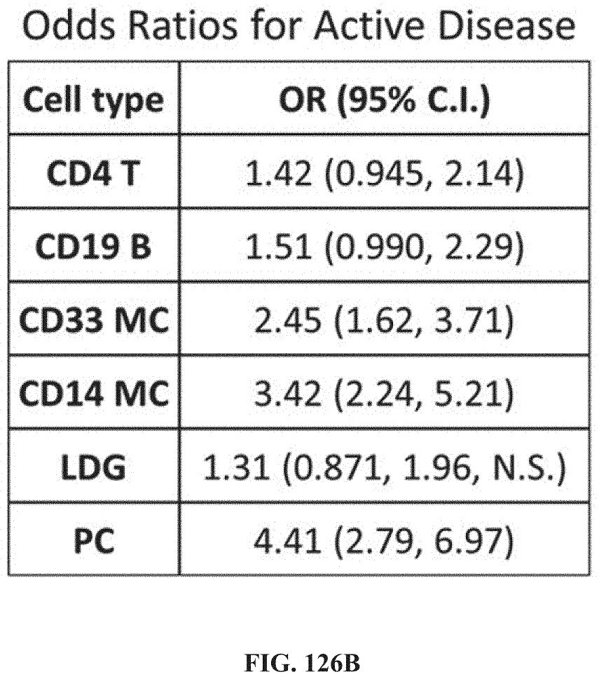

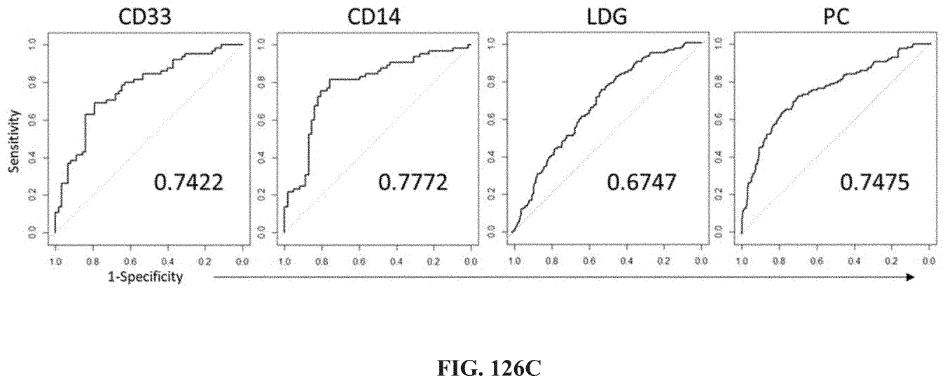

Hierarchical clustering of GSVA scores indicated that enrichment of some modules (PC, CD14+ MC) was more frequently observed in active compared to inactive SLE, although complete separation of active versus inactive samples was not achieved. To explore this in greater detail, odds ratios (OR) for the likelihood of the enrichment of various WGCNA modules from different cell types in active SLE were calculated by comparing the expected versus observed enrichment of each module. As expected (since increased PC are associated with disease activity), PC modules manifested the highest OR for active disease at 4.41, whereas LDG modules exhibited the lowest OR (1.32), consistent with the previous observation that increases in LDG activity do not correlate with disease activity in SLE (FIG. 126A-126B). Notably, MC modules outperformed either CD4 T cells (OR: 1.42) and CD19 B cells (1.51), with CD14+ MC exhibiting a higher OR (3.42 vs 2.45). GSVA scores were then used as input for a Generalized Linear Model-based machine learning algorithm which attempted to identify whether samples from the WB and PBMC test set were obtained from active or inactive SLE patients. CD33 and CD14 MC signatures surpassed LDG signatures and performed at least as well as PC signatures in accuracy as measured by the area under the resulting ROC curves (FIG. 126C).

FIG. 126A shows that GSVA was utilized to generate scores to assess enrichment of WGCNA lymphocyte subset gene modules correlated with disease activity in WB or PBMC samples separated into inactive or active SLE patients. Results are shown following unsupervised hierarchical clustering. The expected and observed correlations to disease states of each module and the cell type of their origin are shown on the right (black: positive correlation; gray: negative correlation; white: unknown correlation; x: no significant correlation). FIG. 126B shows that Odds ratios (OR) with 95% confidence intervals (CI) were calculated from the GSVA data to determine the strength of association of each cellular module with active disease. FIG. 126C shows ROC curves displaying representative results of disease activity prediction by the generalized linear model algorithm for modules from an individual cell type. Area under the curve is shown on each panel.

As shown by the results above, a comprehensive, bioinformatic approach was developed to identify cell type-specific patterns of genetic variation among active and inactive SLE patients and to identify high-priority candidate compounds for drug repositioning efforts. Whereas bioinformatic analysis is often used to supplement studies of SLE pathogenesis in murine models or in vitro, the work described herein represents a “big data” strategy of applying these techniques to patient-derived data in order to identify constellations of genes that might determine clinical outcomes in specific patients.

The initial findings indicated that MC expressed a considerable number of DE genes in both active and inactive patients compared to healthy controls, whereas B and T cells only expressed a significant DE gene signature in active patients compared to healthy controls These findings lead to a hypothesis of a critical role for MC in human SLE, in agreement with studies in lupus-prone mice. B and T cell activity along with that of MC contribute to disease activity in SLE, whereas the altered genomic signatures of MC may preserve the disease state of inactive SLE between flares and may even affect the transition between active and inactive SLE.

The analyses of M1 and M2 signatures indicated that although there is overlap, M2 gene expression is more prominent in inactive SLE patients whereas M1 gene expression is highly enhanced in active SLE patients. This confirms the roles of Mϕ polarization and DC activation in SLE-like conditions: overabundance of proinflammatory M1 Mϕ and decreased expression of the M2 marker CD206 may be detected in both lupus-prone mice and SLE patients, and therapeutic stimulation of M2 polarization may significantly decrease disease severity in an induced murine SLE model. However, experimental intervention in M2b polarization as well as microRNA array profiling demonstrate that M2b Mϕ may contribute to SLE severity, indicating that the relationship between Mϕ polarization and lupus progression is more nuanced than it appears at first glance.

Use of GSVA to compare expression patterns against the BIG-C database revealed differences in upregulated pathways in MC derived from active and inactive SLE patients that minor and reinforce the M1/M2 signatures observed in the DE genes. As expected in SLE, MC from both active and inactive patients are enriched for categories related to IFN signaling and inflammation compared to healthy controls. In contrast, MC from active patients uniquely downregulated pathways related to mitochondrial function and glycolysis in favor of immune cell surface markers and secreted factors, while MC from inactive patients downregulated genes in the cell surface category and are enriched for ubiquitination and sumoylation pathways. These data indicate that MC from active SLE patients favor pro-inflammatory M1-related pathways while MC from inactive patients favor M2-related pathways involved in resolution of the immune response.

Upstream regulator analysis using IPA further confirmed this conclusion, identifying several M2-associated factors as positive regulators in MC from inactive SLE patients but not active patients, including IL-3, IL-4, and HIF1A (FIG. 124). Interestingly, the upstream regulator with the strongest differential z-score preference for active MC versus inactive MC was also the only M2 gene identified as an exclusive regulator for active patient MC: RICTOR, an mTORC2 component RICTOR previously shown to inhibit M1 polarization. This result may simply reflect an expected component of the elevated inflammatory profile of an SLE patient compared to a healthy patient or it may suggest a specific role for RICTOR and the mTORC2 complex in the transition between inactive and active SLE.

Attempting to identify biological upstream regulators empirically by matching gene knockdown and overexpression results from the LINCS analysis platform, on the other hand, revealed practically no polarization-related genes despite identifying several regulators unique to the inactive or active cohorts (FIG. 125). Despite this, these results greatly expanded the potential list of upstream regulators and may suggest pathways with a unique and yet undocumented role in macrophage polarization. Furthermore, these findings extend to the targets and compounds predicted to be useful by LINCS in reverting the gene signatures of active or inactive SLE patients back to the baseline of healthy controls (Table 68 and Table 69). Although unique targets and compounds were identified for active and inactive SLE patients, these did not follow a clear pattern of M1- or M2-related inhibitors. This, along with the lack of polarization genes among LINCS BURs, may in part be related to the inception of the LINCS project as a search for cancer treatments, resulting in a preference for antiproliferative drugs and a higher sensitivity to genes that control proinflammatory signaling pathways. Nonetheless, the presence of both shared and unique targets indicates that this approach can be used either to identify drugs with the potential to treat the SLE signature as a whole or to find therapies tailored toward the presentation of an individual patient's disease. The novel drugs and targets resulting from this analysis may be individually evaluated, screened, and tested to confirm efficacy in SLE treatment.

These analyses were all performed within the same two GEO datasets (GSE10325 and GSE38351). As a result, overlapping findings may have somewhat limited value for the purposes of validation. The results obtained from ML analysis, therefore, presented two critical insights. First, ML findings confirm that while PC genomic signatures correlate with disease activity, LDG genetic signatures do not (FIG. 126B). Second, the construction of a test set from GEO datasets unrelated to the initial analyses allowed for the ML approach to act as an impartial, external validation of findings and conclusions regarding the impact of MC populations on SLE initiation and pathogenesis. Together, these confirmatory results validate the use of ML as a predictive (and potentially diagnostic) tool in SLE research and treatment.

Despite the prevalence of SLE and the considerable studies of the link between gene expression and SLE activity, there remains no definitive diagnostic tool available to determine either whether a patient has SLE or whether or when a patient may experience a flare. Extreme variation among SLE patients further complicates the issue: unsupervised hierarchical clustering of GSVA enrichment scores for disease-associated WGCNA modules produced no uniform pattern of association with SLE activity, and when performed again on pre-sorted datasets, each produced a small subgroup of patients whose enrichment highly resembled that of the other (FIG. 126A). These overlapping groups were initially hypothesized to represent patients with intermediate SLEDAI scores in the process of transitioning between active and inactive disease, but this did not turn out to be the case, highlighting the degree of patient heterogeneity present in the test set and the need for development of computationally intensive, multivariate analysis methods. Data presented here from integration of the datasets into a predictive ML algorithm indicate that MC-derived gene signatures may be used to predict disease activity as reliably as PC signatures which, unlike LDGs, may correlate with disease activity (FIG. 126B-126C). These early MC signatures may provide the basis of a tool to diagnose SLE in its early stages (before PC expansion) or to detect alterations in MC that precede a flare. Subsequent experiments may be performed to further refine and expand the ML approach to include MC samples from a larger cohort of patients.

MC genomic signatures correlated with and successfully predicted SLE disease activity. Whereas B and T cells only manifested DE genes in active SLE patients, DE genes were detectable in MCs from patients with both active and inactive SLE when compared to healthy controls. Examination of these signatures by multiple approaches confirmed the involvement of previously reported pathways (IFN signaling, inflammation, TLR/DAMP signaling) and also identified MC polarization-related pathways and genes as correlated with SLE activity. When used as input for an ML-based prediction algorithm, these MC-derived signatures were used to successfully predict active versus inactive SLE patient samples, and such predictions were more effective compared to using signatures from CD19 B cells and CD4 T cells. The predictive power of these MC signatures makes them compelling input data for perturbagen databases, enabling identification of promising novel and personalized treatment options for SLE.

### Example 35: DE Analysis of Plasma Cells to Identify Genes Associated with Disease Activity in Systemic Lupus Erythematosus (SLE)

Systemic lupus erythematosus (SLE) is a multi-organ autoimmune disease which results in the onset of systemic inflammation and the production of pathogenic, self-reactive autoantibodies. SLE may be highly heterogenous with a wide range of presentations, and progression of the disease can lead to involvement of the skin, lungs, heart, joints, and kidneys, with approximately 40-70% of patients developing lupus nephritis. Several immune cell types may be found to be dysregulated in SLE, and both the innate and adaptive immune responses may be implicated in SLE pathogenesis. The presence of autoreactive antibodies, however, may be a significant factor in the development of severe tissue damage in SLE, making B cells a primary target for study and intervention.

Under healthy conditions, mature B cells residing in secondary lymphoid organ B cell follicles may need exposure to activating stimuli in order to differentiate into antibody secreting cells (ASC) and ultimately plasma cells (PC). B cells may become activated in response to antigen in a T cell-dependent or cell-independent manner; of these, the former leads to an early extrafollicular (EF) response that induces proliferation, class switch recombination, and differentiation into short-lived plasmablasts (PB) that secrete low-avidity antibodies. Activated B cells that re-enter the follicle and interact with T follicular helper cells (via CD40 and ICOS) may form highly proliferative germinal centers (GC) which may produce memory B cells as well as high-affinity, long-lived plasma cells. This process may induce vast, coordinated changes in gene expression as the B cell signaling program (PAX5, BACH2, BCL-6, PU.1, OBF1) is silenced and ASC-specific regulators (IRF4, BLIMP-1, XBP1) are induced. Defects in GC response regulation may be observed in SLE, including loss of follicular exclusion (allowing autoreactive B cells to re-enter follicles and initiate GC reactions), de novo derivation of autoantibodies within the GC by somatic mutation from non-autoreactive precursors, and dysfunctional GC B cell selection and survival.

Studies investigating PC dysfunction in SLE may make them clear targets for therapeutic intervention; however, the complex nature of the B-to-PC signaling program combined with the inherent heterogeneity of SLE may confound these studies. As a result, “big data” and bioinformatic approaches may become a useful strategy to grapple with the number of variables at play, and may show success for promising therapies directed at SLE PC. Here, a comprehensive, bioinformatically-driven approach is performed to use these techniques to address three main aims: first, to identify genetic signatures that define SLE PC subsets and determine whether PCs can be detected in SLE patient tissues; second, to interrogate pathways involved in PC generation in SLE; and third, to highlight key genes and pathways that can be matched to known inhibitors and biologics to accelerate drug repositioning efforts.

Isolation of PC DE profiles from Published Microarray Profiles was performed as follows. DE microarray profiles of both healthy tonsil PC and circulating SLE PC were used for all analyses. Probes were translated into Entrez gene IDs using Affymetrix HG-U133A CDF (Release 36). Non-specific probe IDs were removed. For genes with multiple probe IDs, the DE value with the highest magnitude was used. The lists of Tonsil PC DE genes and SLE PC DE genes were cross-referenced to produce lists of genes that were shared between the two and genes unique to either source.

Functional characterization of DE PC gene signatures and pathway identification was performed as follows. Fisher's Exact Test was used to test for either enrichment or under enrichment of BIG-C functional categories in each gene list. Genes that were DE in SLE patients compared to healthy controls from CD33+ myeloid cells (GSE10325), CD14+ monocytes (GSE38351), or CD4+ T cells (GSE10325) were filtered out of the input DE datasets to produce a focused PC signature. These filtered DE lists were used for all subsequent analyses. Statistical tests were performed in R Version 3.5.1. Signaling molecules and transcription factors upstream of DE genes were identified using IPA Upstream Regulator (UR) analysis. For each regulator, an activation z-score was calculated strictly from experimentally observed information provided for the downstream targets, and an overlap p-value was calculated through Fischer's exact test.

Network analysis and visualization were performed as follows. Visualization of protein-protein interactions and relationships between genes within datasets was done using the Cytoscape (V3.6.0) software and the stringApp (V1.3.2) plugin application. The Clustermaker2 Ap p(V1.2.1) plugin was used to create clusters of the most related genes within a dataset using a network scoring degree cutoff of 2 and setting a node score cutoff of 0.2, k-Core of 2 and a max depth of 100.

PC signature enrichment in tissue of SLE patients was performed as follows. SLE patient microarray DE data from PBMC (FDABMC3, GSE50772, GSE81622), WB (GSE39088, GSE49454), skin (GSE52471, GSE72535), synovium (GSE36700), and kidney (GSE32591) were queried for logFC of genes present in the PC signature as defined by ISCOPE. GSE4588 and GSE10325 were used as positive controls. Fisher's exact test was used to test for the enrichment of SLE unique and common PC DE signatures in tissue and periphery data sets. Due to differences in the number of genes present across platforms, the universal gene number used in these enrichment analyses was the number of genes that could be detected by both chips (Illumina HT-12 V. 4.0, Affymetrix HG-U133 Plus 2). All statistical tests were performed in R Version 3.5.1.

Gene set variation analysis (GSVA) was performed as follows. GSVA (V1.25.4) software package for R/Bioconductor was used as a non-parametric, unsupervised method for estimating the variation of pre-defined gene sets in patient and control samples of microarray expression data sets. The input for the GSVA algorithm was a gene expression matrix of log 2 microarray expression values and a collection of pre-defined gene sets. Enrichment scores (GSVA scores) were calculated non-parametrically using a Kolmogorov Smirnoff (KS)-like random walk statistic and a negative value for a particular sample and gene set. Significance of functional enrichment was calculated using a chi-squared test and categories with p-values less than 0.05 were considered significantly enriched.

Drug target prediction and identification were performed as follows. Queries of the perturbation database from the Broad Institute Library of Integrated Network-Based Cellular Signatures (LINCS) were used to predict potentially useful therapeutic compounds and to confirm the dysregulation of upstream target genes in SLE patient MC by assessing signatures of significantly up- and down-regulated genes for input to the lincscloud API (data.lincscloud.org.s3.amazonaws.com/index.html). Additional drugs to target genes and pathways of interest were identified through the CoLTS and STITCH databases.

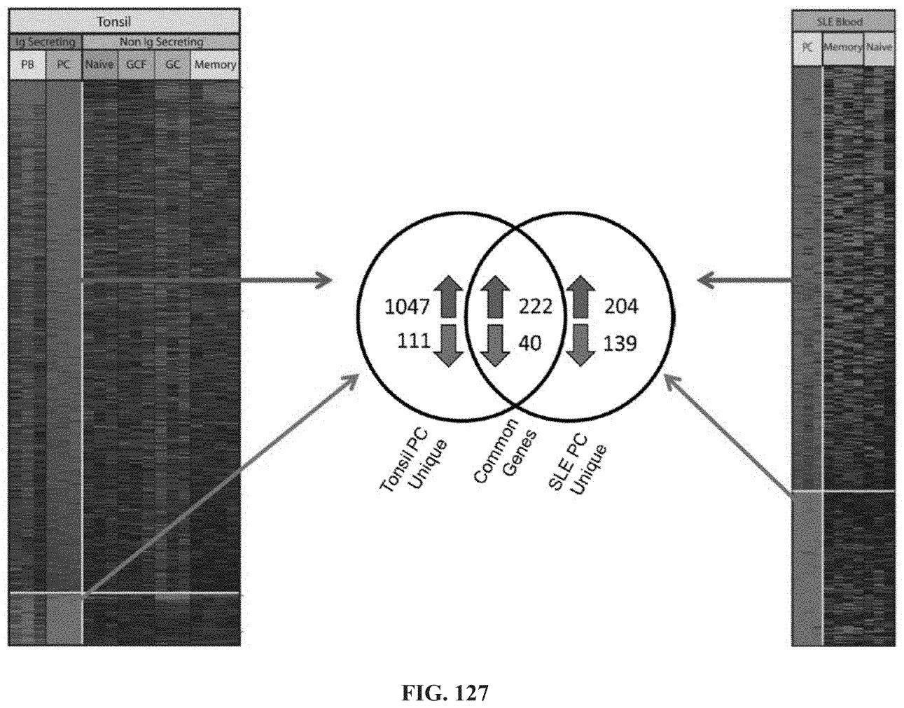

Isolation of PC DE profiles from Published Microarray Profiles was performed as follows. Differential expression data was compiled to generate a dataset appropriate for characterizing unique gene expression profiles of PCs from patients with SLE. Probe IDs and log fold change values for SLE PCs and Tonsil PCs (isolated from healthy controls) were determined. In order to ensure that the most complete and accurate version of the data was used for reanalysis, probes from these lists were updated into current gene IDs using the most recent Affymetrix HG-U133A CDF (Release 36). The two resulting updated lists were then cross referenced to identify unique and shared genes (FIG. 127). The lists contained 262 shared genes (222 upregulated, 40 downregulated), leaving 1158 genes uniquely present in the Tonsil PC list (1047 upregulated, 111 downregulated) and 343 genes uniquely present in the SLE PC list. The diagram in FIG. 127 shows the two figures from which these datasets were represented.

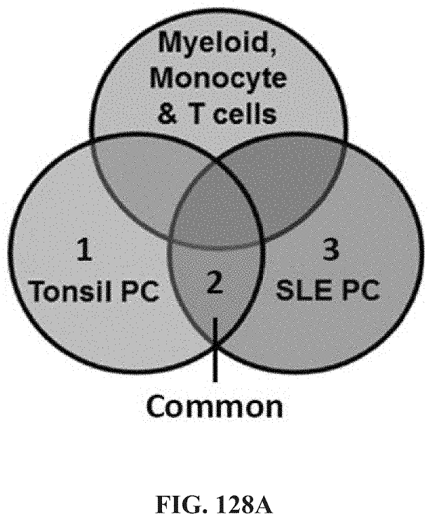

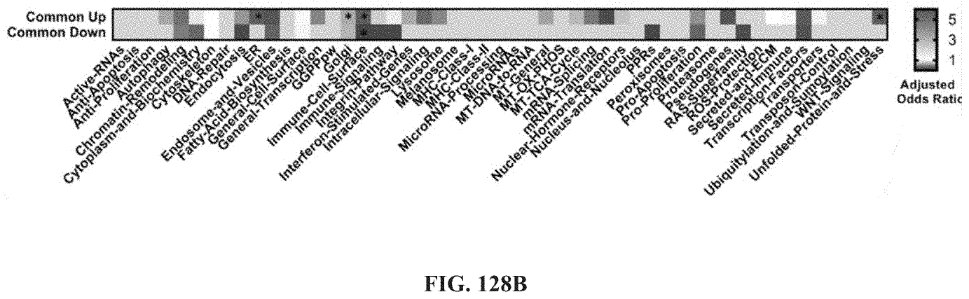

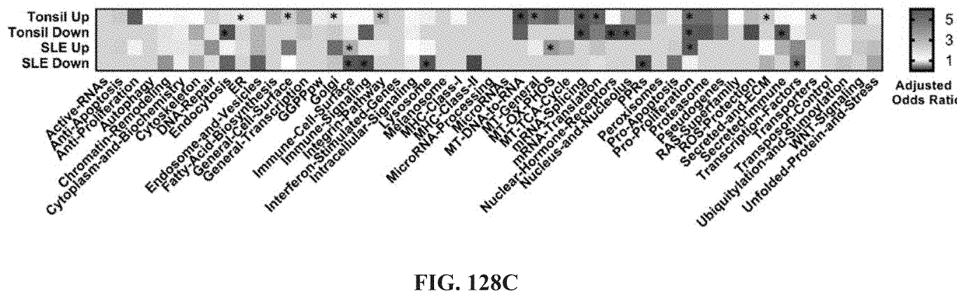

Functional characterization of DE PC gene signatures in SLE was performed as follows. In order to further refine the analysis to include only PC-specific gene signatures, DE gene data was compiled from CD33+ myeloid cells (GSE10325), CD14+ monocytes (GSE38351), and CD4+ T cells (GSE10325) into a filtering list. Any DE genes found in these three datasets were subtracted from the PC DE data, and this filtered PC dataset was used for all subsequent analyses (FIG. 128A). The DE lists referred to as tonsil PC, Common, and SLE PC map to sections 1, 2, and 3 in the Venn diagram, respectively.

First, the functional differences represented by the unique DE gene signatures in SLE PC as compared to healthy (tonsil) PC were characterized. To accomplish this, BIG-C gene annotation analysis was performed. The significantly enriched BIG-C categories found in the common DE gene signature included ER, Golgi, Immune Cell Surface, and Unfolded Protein and Stress (FIG. 128B). Though not significant, the categories Endocytosis, Immune Signaling, Integrin Pathway, mRNA translation, PRRs, Pro-Proliferation, and Transcription Factors were strongly underrepresented in the Common DE list. This shared DE signal between healthy tonsil PC and SLE blood PC defines a core PC profile and was used to track the presence of PCs in various compartments in later analyses.

Among the unique Tonsil PC DE genes, the ER, General Cell Surface, Golgi, Integrin Pathway, Secreted and ECM, and Transporters BIG-C category ORs were significantly enriched while the Endocytosis, Mitochondrial DNA-to-RNA, Mitochondria General, mRNA Splicing, mRNA Translation, Nuclear Hormone Receptors, and Nucleus and Nucleolus BIG-C categories were significantly underrepresented (FIG. 128C). The Pro-Proliferation BIG-C category OR was also significantly enriched in SLE PCs, as was the Mitochondrial Ox-Phos category.

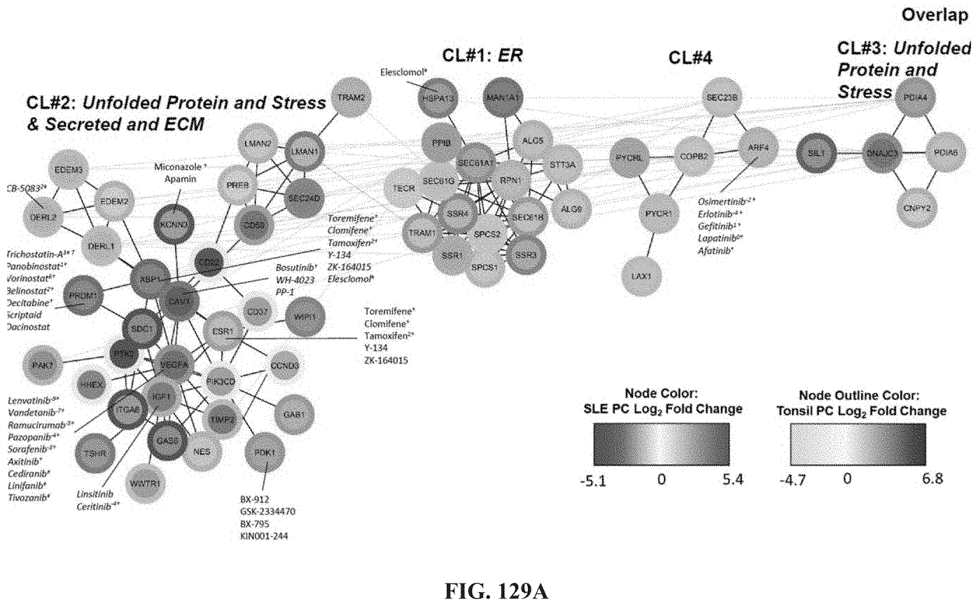

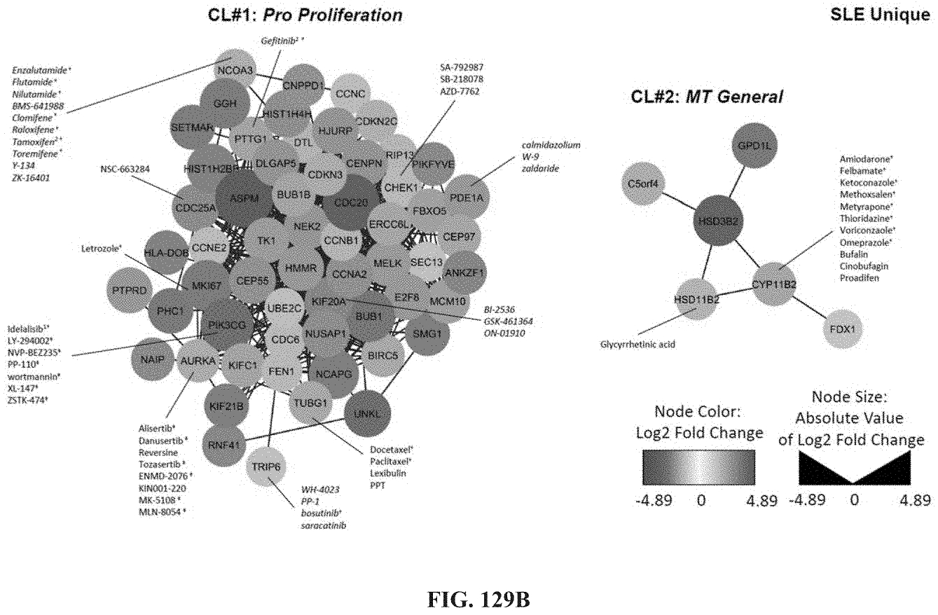

Next, protein interaction-based clustering of SLE PC and SLE/Tonsil Common DE genes was performed as follows. A more detailed analysis of the composition of the Common DE and SLE PC gene signatures was performed by interrogating protein-protein interaction network clusters. The DE genes common to the SLE PC and Tonsil PC datasets formed four discrete clusters: a large unfolded protein response/secreted protein cluster, an ER cluster, a small unfolded protein response cluster, and a small cluster with undefined function (FIG. 129A). The SLE PC DE list produced only two clusters via MCODE analysis: one large cluster centered around pro-proliferation signaling pathways, and one small cluster containing ER- and mitochondria-related genes (FIG. 129B). While individual intensities varied, the logFC direction for each clustered gene was preserved across both the SLE and Tonsil PC datasets (FIG. 3A).

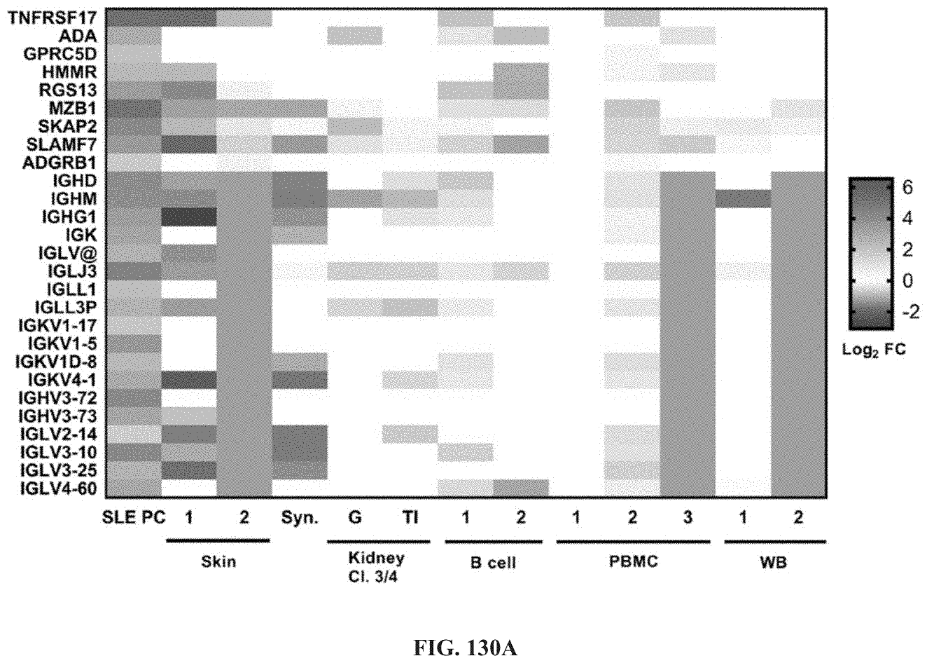

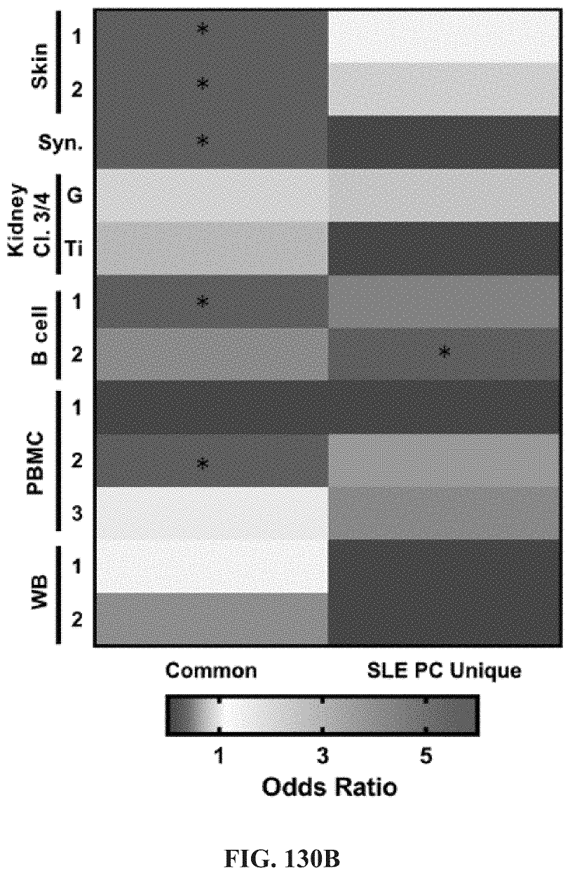

The PC DE signature was tracked in the periphery and tissues of SLE patient via microarray data. Aside from the presence of PC in SLE kidney, the distribution of PC within tissues of human SLE patients is not clearly defined. A large curated database of SLE patient tissue gene expression was leveraged against the PC-derived gene signatures extracted from the data collected to map these PC subsets to diseased tissues. Expression changes of genes overlapping between the ISCOPE PC signature and the SLE PC DE data were analyzed across several tissue (skin [GSE52471, GSE72535], synovium [GSE36700], kidney glomerulus and tubulointerstitium [GSE32591]) and peripheral cell (B cell [GSE4588, GSE 10325], PBMC [FDABMC3, GSE50772, GSE81622], and WB [GSE39088, GSE49454]) microarray datasets from SLE patients. Many of these genes were found to be upregulated most in the skin and synovium, followed by the kidney and B cell datasets, with some expression detected in the PBMC and WB datasets (FIG. 130A). Some of the microarray platforms used for these studies did not include immunoglobulin genes, however, eliminating the potential to detect a good portion of the ISCOPE PC definition profile (gray cells, FIG. 130A). Using the SLE PC and Common PC DE gene lists revealed enrichment patterns of divergent subsets of the PC signature across different SLE tissue and peripheral cell datasets (FIG. 130B). ORs for the Common PC signature were strongly enriched in nearly all interrogated datasets, while the SLE PC unique signature was enriched mostly in the PBMC and skin datasets. As expected, ORs for both the Common and SLE Unique PC signatures were enriched in the B cell datasets (positive controls).

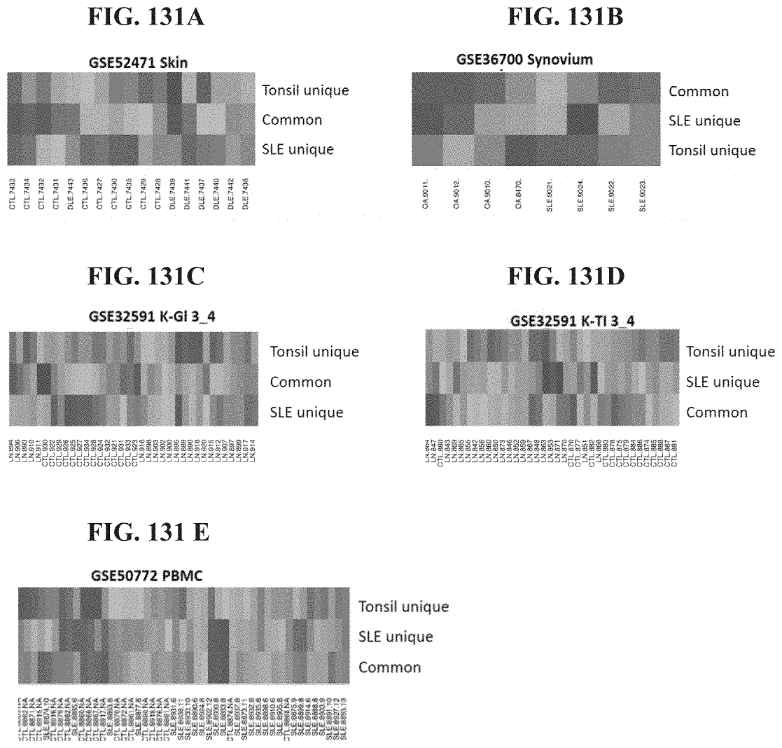

To further examine and confirm these findings, GSVA was used to determine enrichment of the Tonsil PC, SLE PC, and Common signatures in tissue (FIG. 131A-131D) and PBMC samples (FIG. 131E) from SLE, DLE, LN, and OA patients. Enrichment of the Common and SLE PC signatures only appeared to successfully identify and sort DLE, SLE, and LN patient samples in the skin, synovium, and kidney glomerulus, respectively (FIG. 131A-131C). Of interest, enrichment of the SLE PC and Common signatures was also able to successfully subset samples from patients with legional DLE separately from patients with nonlegional DLE. LN patient samples were less cleanly identified from healthy control samples when these signatures were applied to the kidney tubulointerstitium, but the Common signature tended to be enriched in LN patient samples while the Tonsil PC signature (representing homeostatic/healthy PC gene signaling) tended to be enriched in the control samples (FIG. 131D). PBMC samples, on the other hand, were not successfully discriminated by cohort according to GSVA enrichment of the Tonsil PC/SLE PC/Common signature paradigm (FIG. 131E).

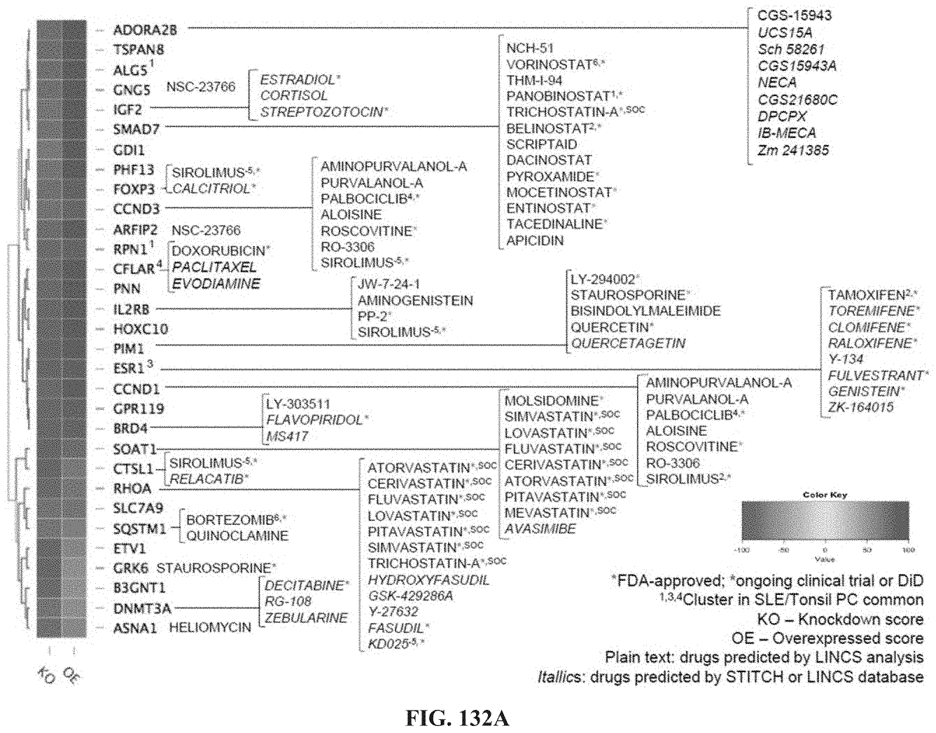

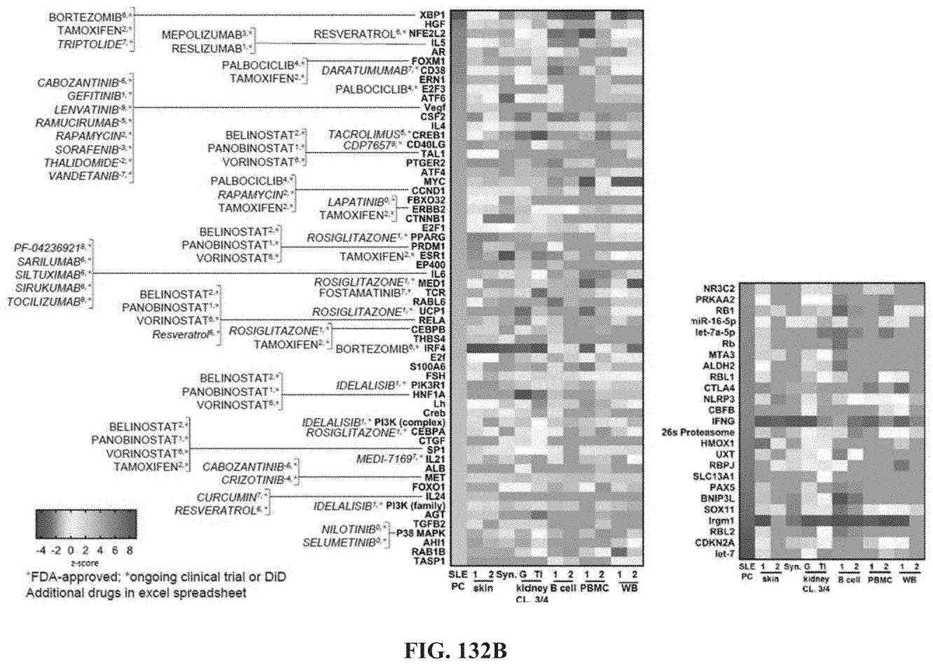

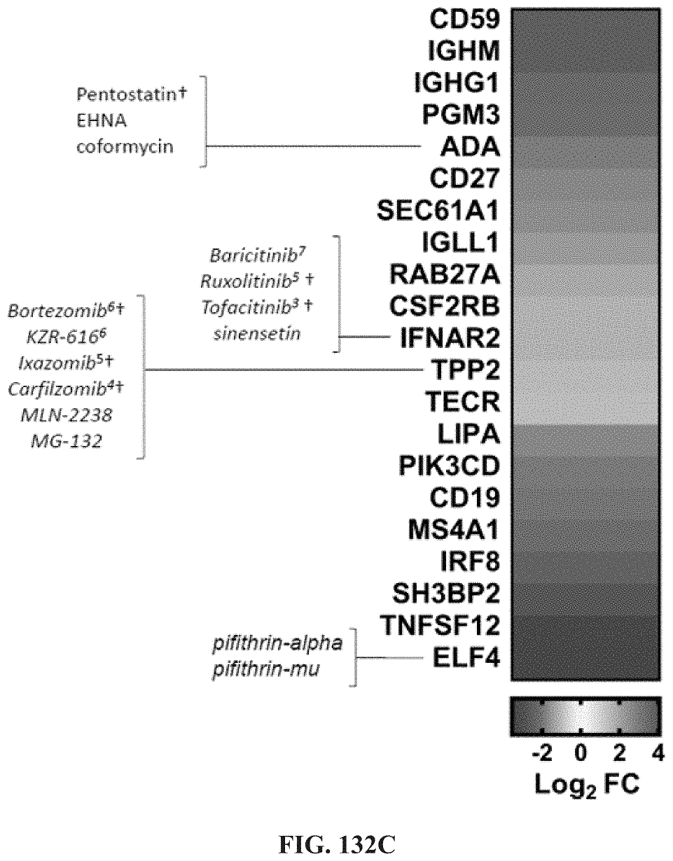

Upstream regulators of SLE PC DE gene signatures cluster in proliferation and cell cycle checkpoint pathways were analyzed as follows. IPA upstream regulator analysis was used to further distill the SLE PC DE signature and identify keystone genes and signaling pathways. Total SLE-related PC DE gene data (SLE PC plus Overlap DE signatures, regions 2+3 of FIG. 128A) were used as input for IPA, producing 85 upstream regulators with activation z-scores≥|2| that were found to be significantly differentially expressed in additional SLE tissue and peripheral cell datasets (FIG. 132B). Of these, 60 were predicted to be activated and 25 were predicted to be inhibited. As expected, several high-scoring regulators were related to B/PC differentiation (XBP1, ILS, IL6, CD40LG, PRDM1, IRF4) (FIG. 132B). Most of the upstream regulator activation predictions by IPA corresponded to observed gene expression in tissue and peripheral cell samples from SLE patients, save for two strongly contradicting genes: IRF4, while predicted as activated, was strongly downregulated in SLE sample gene expression, and IFNG was highly expressed in all samples yet predicted to be inactivated by IPA (FIG. 132B). These genes may represent two signaling pathways that behave in a predictable manner in homeostasis but are highly dysregulated in lupus.

To determine what portion of upstream regulators were derived from the SLE PC-specific portion of the SLE Total gene signature, the SLE Unique PC DE data (region 3 of FIG. 128A) was also used as input for IPA upstream regulator analysis. Of the 48 upstream regulators with activation z-scores≥|2|, 23 were present in the regulators derived from the SLE Total signature (FIG. 132B) and 25 were new predictions. Many of the genes predicted by IPA to be upstream regulators of the SLE PC and Common PC signatures clustered into a conserved set of overlapping signaling pathways, including GC activation and PC differentiation, Ig production and ER stress response, and high representation of oncogene/cell cycle checkpoint mechanisms.

These pathways, along with representative upstream regulators of the SLE PC and Common PC signatures, were assembled into a model of potential mechanisms of SLE PC differentiation and function. IPA canonical pathways that coincide with upstream regulator interactions were determined.

PCs may be a primary driver of SLE pathogenesis and may be important targets of efforts to advance SLE treatment design and drug development. The drugs that result from these efforts, however, tend to be less efficacious than expected from the prevalence of PCs in SLE patients and are often only effective in specific patient cohorts. This has in turn lead to the realization that PCs represent a heterogenous group of targets from patient to patient rather than a single monolithic cell population, and efforts to advance SLE treatment targeting PCs must adapt accordingly. To this end, a comprehensive bioinformatic approach was employed to mine genomic data from SLE patient PC samples in order to identify phenotypic subpopulations of PCs in SLE, track these subpopulations across diseased tissues, and predict novel high-impact molecular targets and potential therapeutic compounds for fast-tracked repositioning.

Alarge set of differential expression data derived from PC sorted from SLE patient samples was generated. DE comparisons from this analysis identified key differences between circulating SLE PC and bone marrow PC as well as overlapping gene signatures between tonsil PC and SLE PC. Separating these compiled DE data into SLE unique, tonsil unique, and common lists and filtering out potentially confounding gene signatures from other cell types allowed a more fine characterization of these signatures via bioinformatic approaches (FIG. 128A).

Categorization of DE genes via BIG-C enables the calculation of odds ratios and identification of significantly enriched biological functional categories within each PC signature. Certain BIG-C categories resulted in consistent enrichment significance and directionality across all three lists, including ER, Golgi, Endocytosis, Integrin Pathway, and mRNA translation. Interestingly, while the Pro-Proliferation category was strongly underrepresented in the Common signature, it was significantly enriched in the unique SLE PC signature (FIG. 128C), consistent with observations of short-lived, autoantibody-producing PCs in the NZB/W mouse model. Significant enrichment of the Mitochondiral Ox-Phos category, however, is more suggestive of long-lived PCs, which typically undergo little to no proliferation. SLE PCs may represent an abnormal phenotype unlike either of these canonical PC populations, producing high levels of autoantibody and undergoing excessive proliferation while also persisting for extended periods of time. Protein interaction-based clustering by MCODE produced similar results, confirming that the DE genes found in the filtered unique SLE PC list interact with each other in signaling pathways identified by BIG-C: proliferation, protein secretion, and mitochondrial function (FIG. 129B).

The Immune Cell Surface and Unfolded Protein/Stress BIG-C categories, which were significantly enriched specifically in the Common list, represent the core of the conserved PC phenotype: high expression of immunoglobulin genes and the unfolded protein response induced by their translation and secretion (FIG. 128B). This signature was used to track the presence of PCs across tissues by cross referencing with other datasets using the ISCOPE tool. This analysis revealed the skin and synovium to be the most highly infiltrated reservoirs of PC in SLE patients, followed by the kidney (FIG. 130A). Considerably less enrichment was detected in SLE patient PBMC and WB samples, providing a possible mechanism to describe the limited effectiveness of intravenously administered anti-PC therapies.

Next, it was determined whether the PCs detected in these tissues were more genetically similar to the signature observed from SLE PCs or the shared Common PC signature. OR calculation showed that while high levels of Common signature-enriched PCs appeared to accumulate in the synovium, enrichment of the SLE PC signature was confined to the skin and the circulation (FIG. 130B). Verification of these findings via GSVA query of additional datasets revealed that the Common and SLE PC signatures were able to identify and sort samples from patients with various presentations of lupus obtained from the skin, synovium, and kidney, mirroring the results obtained by BIG-C OR calculation (FIG. 131). It is of interest to note that these tissues have been found to express high levels of a proliferation-inducing ligand (APRIL), contributing to autoantibody secretion in the joints of RA patients and kidneys of NZB/W mice, and potentially contributing to the enrichment of Pro-Proliferation genes observed in BIG-C analysis of the SLE PC signature (FIG. 128C). By interrogating the expression of immunoglobulin genes in each of these tissue datasets we could also estimate PC maturity. Both heavy chain as well as kappa and lambda light chain Ig genes were upregulated in the SLE PC and Tonsil PC signatures. IGM heavy chain and light chains (kappa, IGKC and lambda, IGLJ3) were upregulated in the blood samples and all four tissue datasets, whereas IGHG1 was upregulated in the blood, synovium & TI and downregulated in the skin (FIG. S3B). The presence of both IgK and IgL and numerous VL genes indicates that the PC infiltration in these tissues is polyclonal. Pre-switch (IgM+/IgD+) PCs were found in the periphery, skin, and synovium, and may be contributing to the observed Pro-Proliferation signal in the SLE PC signature. IgM+/IgD− PCs were found in the glomerulus, while IgG PCs were found in synovium, TI, and periphery.

Characterization of DE signatures via IPA upstream regulator analysis and canonical pathway analysis highlighted further functional insights into SLE-specific PC dysfunction. The upstream regulators derived from the Total SLE PC DE list (FIG. 132B) did emphasize genes critical to regulation of PC differentiation and maturation as expected (e.g. XBP1, PRDM1, IL5), but these genes were not identified as upstream regulators for the SLE PC unique DE signature, confirming the role of the Common DE signature as a generic PC identification and tracking signature. The Total SLE- and SLE PC-derived upstream regulators both also contained strong, overlapping evidence for the enrichment of signaling pathways related to Ig production and ER stress response, cell cycle checkpoint genes and oncogenes, and various cell proliferation pathways (FIG. 132B). The first of these reflects the well-documented tendency of SLE PCs to be highly active protein factories, overproducing pathogenic autoreactive antibodies and inducing homeostatic stress responses (which itself may be a fruitful axis of therapeutic intervention). Several of these pathways, when taken together with the presence of polyclonal pre-switch PC signatures in tissues, reflect a population of PCs in SLE that have many qualities of plasmablasts; unlike a terminally differentiated healthy PC, SLE PCs retain pathway signatures as if they are recently emerged from germinal centers (IL-5/6, FOXM1, E2F1, CDKN1A) and sustain activation of proliferative pathways (CSF2, CD38, MYC, VEGF). Several upstream regulators also highlight the importance of the feedback loop between PC and Th17 responses in SLE (TCR, CD40L, AREG, PTGER2, miR-21, AGT), and may provide the key to dysregulation of IFNg signaling reflected in conflicting DE gene expression values and its IPA activation z-score (FIG. 132B).

Finally, the signatures derived from the filtered DE lists were used as input for the target prediction and drug discovery analysis pipelines in order to bioinformatically generate lists of promising compounds and therapeutics. High-priority targets were generated via IPA upstream regulator analysis (FIG. 132A) and by cross-reference with the AMPEL Primary Immunodeficiency Gene Database (FIG. 132B), which identifies and catalogs keystone genes that act as checkpoints in the development of autoimmunity and protect against gross failure of immune tolerance. These targets are matched to specific biologics and small molecule inhibitors that have been suggested by LINCS connectivity score analysis (Table 70) or identified as therapeutics of interest via the LxrL-STAT and CoLTS initiatives (FIGS. 132A-132B). While many of the compounds identified this way are already standard-of-care drugs for clinical management of SLE, several novel drugs in development and FDA-approved drugs were identified by this process, representing promising fast-track candidates for repositioning into clinical trials for SLE patients.

TABLE 70
LINCS connectivity score analysis matching targets to specific biologies and small molecule inhibitors

| | | LINCS CsC Score | | |
|---|---|---|---|---|
| Top Drug | Target | Range | Mean ± SEM | Cluster |
| NCH-51 | HDAC (Vorinostat^{†e}) | (−99.47)-(−97.84) | −98.86 ± 0.29 | SLE 4 |
| SUNITINIB^{†} | Tyrosine Kinase (broad) (Nilotinib^{†c}) | (−99.68)-(−94.49) | −97.73 ± 1.63 | |
| SIMVASTATIN^{†1} | HMG-CoA reductase (Rosuvastatin^{†3}) | (−99.74)-(−94.68) | −97.21 ± 2.53 | |
| DICHLOROBENZAMIL | Ca channel | (−99.32)-(−92.45) | −97.11 ± 1.6 | |
| ETOPOSIDE^{†} | topoisomerase II (Irinotecan^{†-1}) | (−98.77)-(−94.9) | −96.83 ± 1.94 | SLE 9 |
| BIBU-1361 | EGFR (Gefitinib^{†1}) | (−99.7)-(−93.09) | −96.17 ± 1.66 | |
| PREDNISOLONE^{†T} | Glucocorticoid receptor agonist | (−95.18)-(−94.89) | −95.03 ± 0.14 | |
| AMINOPURVALANOL-A | CDK (pan) (Palbociclib^{†4}) | (−94.56)-(−93.53) | −94.04 ± 0.52 | SLE 3 |
| ENTINOSTAT^{†} | HDAC1 | (−97.94)-(−88.9) | −93.42 ± 4.52 | SLE 4 |
| FLUNISOLIDE^{†} | Nuclear receptor subfamily 3 | (−96.88)-(−88.04) | −93 ± 1.22 | |
| TORIN-1 | mTORC1/2 | (−92.26)-(−92.09) | −92.17 ± 0.08 | |
| AZD-8055 | mTORC1 (Everolimus^{†3}) | (−99.29)-(−84.56) | −92.16 ± 3.07 | |
| SELUMETINIB^{‡} | MEK1/2 (Selumetinib^{‡0}) | (−92.69)-(−90.76) | −91.93 ± 0.59 | SLE 9 |
| WORTMANNIN^{‡} | PI3K (pan) | (−95.63)-(−80.07) | −91.85 ± 3.78 | SLE 9 |
| THIORIDAZINE^{†} | Cytochrome P450 | (−92.07)-(−91.03) | −91.55 ± 0.52 | |
| TAMOXIFEN^{†2} | ER (pan) (Tamoxifen^{†2}) | (−99.29)-(−83.88) | −91.41 ± 4.45 | SLE/Tonsil 3 |
| CAMPTOTHECIN^{‡} | topoisomerase I (Irinotecan^{†-1}) | (−97.77)-(−84.17) | −90.97 ± 6.8 | |
| PYRVINIUM-PAMOATE^{†} | Wnt | (−96.94)-(−84.65) | −90.8 ± 6.14 | |
| PI-828 | PI3Kb | (−96.61)-(−84.59) | −90.6 ± 6.01 | SLE 9 |
| GDC-0879 | B-Raf (Vemurafenib^{†-e}) | (−97.42)-(−83.55) | −90.48 ± 6.94 | |
| VAMA-37 | DNA-PK | (−98.37)-(−82.02) | −90.44 ± 2.09 | |
| JAK3-INHIBITOR-VI | JAK3 (Tofacitinib^{†3}) | (−91.31)-(−89.3) | −90.3 ± 1.01 | |
| BMS-536924 | IGF-1R | (−95.5)-(−79.15) | −90.23 ± 3.79 | SLE/Tonsil 3 |
| WZ-4-145 | Dopamine receptor D1 | (−97.14)-(−80.22) | −90.2 ± 1.63 | |
| THIOTHIXENE^{†} | Dopamine receptor D2 | (−96.07)-(−79.35) | −89.99 ± 3.68 | SLE/Tonsil 6 |
| TW-37 | BCL-2 | (−98.47)-(−81.18) | −89.92 ± 2.56 | |
| BENZAMIL | Na channel | (−99.66)-(−79.86) | −89.76 ± 9.9 | |
| NAPROXOL | Prostaglandin E synthase 2 | (−92.16)-(−86.41) | −89.29 ± 2.87 | |
| HSF90-INHIBITOR | HSP90 | (−96)-(−82.65) | −88.92 ± 3.88 | SLE/Tonsil 1 |
| BI-2536^{‡} | PLK1 | (−94.77)-(−83.09) | −88.74 ± 3.38 | |
| RO-04-6790 | 5-HT 6 | (−89.68)-(−87.37) | −88.53 ± 1.16 | |
| TRAMADOL^{†} | Opioid receptor mu | (−93.51)-(−82.43) | −87.97 ± 5.54 | |

| | | LINCS CsC Score | | |
|---|---|---|---|---|
| Top Drug | Target | Range | Mean ± SEM | Cluster |
| QUIFLAPON | Arachidonate 5-lipoxygenase | (−98.27)-(−76.28) | −87.94 ± 3.45 | |
| METHYLENE-BLUE^{†} | Monoamine oxidase | (−93.24)-(−83.34) | −87.25 ± 3.04 | |
| PROFENAMINE | Cholinergic receptor, muscarinic | (−99.51)-(−75.5) | −86.46 ± 4.09 | |
| EPOXYCHOLESTEROL | Nuclear receptor subfamily 1 | (−96.71)-(−74.81) | −86.2 ± 6.34 | |
| AS-605240 | PI3Kg (Idelalisib^{†1}) | (−97.53)-(−74.64) | −86.09 ± 11.45 | SLE 9 |
| PAROXETINE^{†} | SERT | (−98.95)-(−75.29) | −86.03 ± 3.18 | |
| AT-SUMO-1 | SUMO1 | (−86.08)-(−85.16) | −85.62 ± 0.46 | |
| RS-39604 | 5-HT 4 | (−90.77)-(−76.66) | −85.4 ± 3.21 | |
| AVICIN-G | AMPK activator | (−96.08)-(−76.28) | −85.02 ± 2.47 | |
| CEDIRANIB^{‡} | VEGFR (pan) (Sorafenib^{†-3}) | (−89.88)-(−79.38) | −84.63 ± 5.25 | SLE 3 |
| ENMD-2076^{‡} | Aurora kinase A | (−84.56)-(−84.48) | −84.52 ± 0.04 | SLE 1 |
| TRICIRIBINE^{‡} | Akt (pan) | (−92.49)-(−75.11) | −38.8 ± 8.69 | SLE 1 |
| TIPIFARNIB-P2^{‡} | Farnesyl transferase | (−87.67)-(−79.84) | −83.76 ± 3.92 | |
| CHEMBL-1222381 | K channel | (−85.54)-(−81.71) | −83.63 ± 1.91 | |
| BX-795 | PDK1 | (−86.03)-(−80.37) | −83.2 ± 2.83 | |
| METHOTREXATE^{†} | DHFR | (−85.18)-(−81.41) | −83.02 ± 1.12 | |
| CYTOCHALASIN-D | Actin | (−91.35)-(−75.09) | −81.41 ± 3.85 | SLE/Tonsil [symbol] |
| APICIDIN | HDAC3 | (−85.93)-(−76.11) | −81.02 ± 4.91 | SLE 4 |
| BNTX | Sigma receptor | (−84.57)-(−77.15) | −80.86 ± 3.71 | |
| EO-1428 | MAPK | (−84.78)-(−76.92) | −80.85 ± 3.93 | SLE 9 |
| ELLIPTICINE | TP53 | (−81.41)-(−79.47) | −80.44 ± 0.97 | |
| CP466722 | ATM Kinase | (−84.4)-(−76.29) | −80.34 ± 4.05 | |
| MYCOPHENOLATE- | IMPDH1/2 | (−79.97)-(−79.77) | −79.87 ± 0.1 | |
| MOFETIL^{†} | | | | |
| JW-7-24-1 | Lck | (−82.95)-(−76.55) | −79.75 ± 3.2 | |
| PHA-665752 | c-Met | (−85.6)-(−75.1) | −78.86 ± 2.15 | |
| STAUROSPORINE^{‡} | Kinases (broad) | (−76.04)-(−74.59) | −75.31 ± 0.73 | |
| NERATINIB^{‡} | HER2 | (−75.55)-(−74.7) | −75.12 ± 0.43 | |
| CLOBENPROPIT | H3 receptor | (−75.55)-(−74.7) | −75.12 ± 0.47 | |
| ALIMEMAZINE^{‡} | Histamine receptor 1 | (−75.55)-(−74.7) | −75.12 ± 0.46 | SLE 9 |
| H-7^{‡} | PKCa | (−75.55)-(−74.7) | −75.12 ± 0.45 | |
| SA-792987 | Chk1 | (−75.55)-(−74.7) | −75.12 ± 0.44 | SLE 3 |
| TOZASERTIB^{‡} | Aurora kinase (pan) | (−75.55)-(−74.7) | −75.12 ± 0.43 | SLE 1 |

^{†}FDA-approved;
^{‡}ongoing clinical trial of DiD [symbol]
indicates data missing or illegible when filed

For example, baricitinib (FIG. 6C) is a selective, reversible inhibitor of JAK-1 and JAK-2 that was approved by the FDA to treat moderate to severe rheumatoid arthritis (RA), a chronic inflammatory autoimmune disease that shares many characteristics of SLE. Baricitinib may be shown to be well tolerated and highly efficacious in relief of RA symptoms, which, combined with its convenient once-daily oral formulation, make it a good candidate for rapid repositioning. Baricitinib inhibits intracellular signaling transduction via the JAK1/JAK2 pathway, a key regulator of RA pathogenesis as well as several critical immune signaling pathways (including IFNAR2, implicated as a dysregulated PID in the SLE signature [FIG. 132C]). Accordingly, clinical trials of baricitinib in SLE may be undertaken.

Several other targets identified by the big data approach described in this work may therefore be important leads for SLE repositioning. Targets of the proteasome inhibitor family of chemotherapy agents (bortezomib, ixazomib, carfilzomib) were identified as members and regulators of the SLE PC signature by multiple methods (FIGS. 132A-132C), and this family of drugs may show promise in treatment of autoimmune disease. Additionally, bortezomib may be successful in cases of refractory SLE and cases of refractory renal and pulmonary SLE, resulting in significant decreases in serum Ab levels, anti-dsDNA, and circulating PC depletion. Targets of FK506-binding protein (FKBP)-regulated pathways, which are modulated by tacrolimus and sirolimus, were also identified as both DE genes and URs of the SLE PC signature (FIGS. 132A-132B). These drugs form complexes with FKBPs and inhibit mTOR pathway signaling, disrupting IL-2 signaling and cell cycle regulation. Studies in RA, lupus nephritis, and SLE may suggest benefits of tacrolimus and sirolimus treatment in autoimmunity, but follow up trials with larger enrollment and appropriate placebo controls may be performed to fully establish the potential of these compounds.

Several regulators of histone deacetylation (HDACs) and associated pathways were also identified via multiple methods as important players in the SLE PC signature (FIG. 129, FIG. 132, Table 70). HDAC inhibitors (e.g., panobinostat, vorinostat, belinostat) may be commonly used as a component of cancer therapy regimens due to their roles in inducing cell cycle arrest and interfering with posttranslational histone modifications that influence expression of oncogenes and tumor suppressor genes. The combination of the identification of epigenetic factors that contribute to SLE susceptibility and the detection of nucleosome- and histone-targeting autoantibody production by PCs may lead to a hypothesis that HDACi treatment may be a major breakthrough in SLE therapy. Indeed, HDAC expression may be shown to be upregulated in lupus-prone mouse models as well as human SLE patients, and pan-HDAC inhibition can decrease disease in lupus mice. Selective inhibition of HDAC6 in particular may show promise by retaining effectivity while theoretically limiting side effects induced by toxicity of pan-HDAC inhibition. The success of these treatment strategies and the ability of the analysis pipeline described herein to identify and prioritize associated molecular targets demonstrate that such methods represent significant advances to breaking down complicated multivariate diseases with complicated genetic and epigenetic contributing factors, and may serve as integral approaches to developing treatment strategies in SLE.

### Example 36: Analysis of Single Nucleotide Polymorphisms Associated with Systemic Lupus Erythematosus Provides Insights into Molecular Pathways Operative in Ancestral Groups

Individuals of African-Ancestry (AA) may experience systemic lupus erythematosus (SLE) more severely and with an increased co-morbidity burden compared to European-Ancestry (EA) populations. However, the relationship between genetics, molecular pathways and disease severity may not be fully delineated. A comprehensive systems biology approach was applied using bioinformatics and pathway analysis tools to identify the genetic drivers of gene expression networks and key genes within SLE-associated biological pathways. Newly predicted genes were coupled to SLE differential expression (DE) datasets to map dominant molecular pathways representative of each ancestry and available treatments unique to each ancestral group. Pathway validation was provided by gene set variation analysis (GSVA) which identified differentially enriched ancestry-specific gene signatures in SLE patients and control whole blood.

Systemic lupus erythematosus (SLE) may be a multi-organ autoimmune disorder associated with significant morbidity and mortality. SLE may be strongly influenced by genetic factors and recent candidate gene and genome wide association studies (GWAS) may identify over 90 SLE susceptibility loci. However, disease development may be complex and often unpredictable, with considerable differences noted in individuals of different ancestral groups. Some studies may show that individuals of African-Ancestry (AA) experience the disease more severely and with an increased co-morbidity burden compared to European-Ancestry (EA) populations. Moreover, there may be variability in the response of individuals of different ancestral groups to standard medications, including cyclophosphamide, mycophenylate, rituximab and belimumab. For example, belimumab, a monoclonal antibody directed to TNFSF13B may exhibit some clinical benefit in moderately active SLE, but may be reported to be less effective in treating AA populations.

Understanding the functional mechanisms of causal genetic variants underlying SLE may provide essential information to identify ancestry-specific molecular pathways and therapeutic targets relevant to disease mechanisms. Although GWAS has achieved great success in mapping disease loci in polygenic autoimmune diseases, GWAS findings may fail to impact clinical practice. Moreover, for many single nucleotide polymorphisms (SNPs), the biologic implications may not have been identified. Thus, a major challenge lies in understanding the molecular meaning of an association of a single nucleotide polymorphism (SNP) with a disease such as SLE. This process may comprise the identification of causal genes from multiple genetic candidates associated with a lead or “tagging” SNP. This analysis may be complicated by the finding that the majority of SLE-associated SNPs are located outside of protein coding regions. However, a number of approaches can be employed to deconvolute the implications of GWAS findings. For example, utilization of expression quantitative trait loci (eQTL) mapping to identify genetic variants that affect gene expression either in cis (within 1 Mb) or trans (outside of the 1 Mb window or on a different chromosome) can offer important insights into disease causing mechanisms contributing to SLE. In addition, the interactions of transcription factors (TFs) with DNA regulatory elements (e.g. promoters and enhancers) may play a critical role in determining gene expression. However, connecting distal regulatory regions, such as enhancers, with target genes may remain complex. The integration of data from functional genomics, including transcription factor chromatin immunoprecipitation sequencing (ChIP-seq), DNase-Seq, chromosome accessibility sequencing (ATAC-Seq) and chromosome conformation capture-based technologies (such as 4C, 5C, Hi-C, ChIA-PET, HiChIP and Capture Hi-C) may be used to identify variants that may disrupt transcription factor binding site (TFBS) occupancy in active regulatory regions and reliably predict altered downstream target gene expression. Together, these analyses can provide additional information on the molecular implications of GWAS results.

As a hypothesis, the use of multiple orthogonal approaches may provide novel insights into the totality of perturbations in molecular pathways predicted by GWAS results, the possible differences in pathologic mechanisms in different ancestral groups, and also identify novel therapeutic targets. To test this, SLE-associated variants were linked from diverse ancestral populations to potential biologically relevant expression genes (E-Genes) via eQTL analysis. In parallel, SNPs were queried for their potential role as regulatory variants and mapped to their downstream target genes (T-Genes). Finally, SNPs that were neither regulatory nor identified as an eQTL were assigned to the most physically proximal gene (P-Genes). Coding region SNPs associated with deleterious amino acid changes (nonsynonymous or nonsense) were annotated using functional prediction tools. This analysis yielded the identification of 1,904 potential SLE-associated genes divided by ancestry (1,156 European American (EA), 73 African American (AA), and 675 shared between ancestries). A comprehensive systems biology approach was then applied using bioinformatics and pathway analysis tools to identify the genetic drivers of gene expression networks and key genes within SLE-associated biological pathways, including upstream and downstream regulators. Predicted genes were then coupled to SLE differential expression (DE) datasets to map candidate molecular pathways and available treatments unique to each ancestral group. Together, these genetic and gene expression analyses have clarified the fundamental differences in lupus molecular pathways between ancestral populations, identified molecular pathways that are similar or differ between ancestral groups, and have helped identify novel drug candidates that may uniquely impact EA and AA SLE patients.

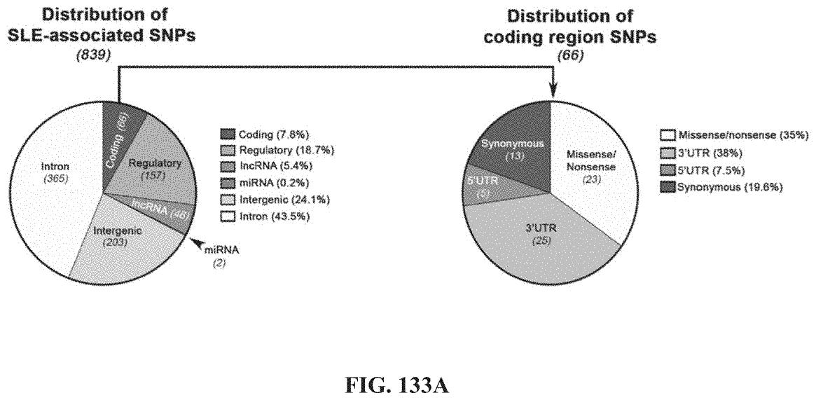

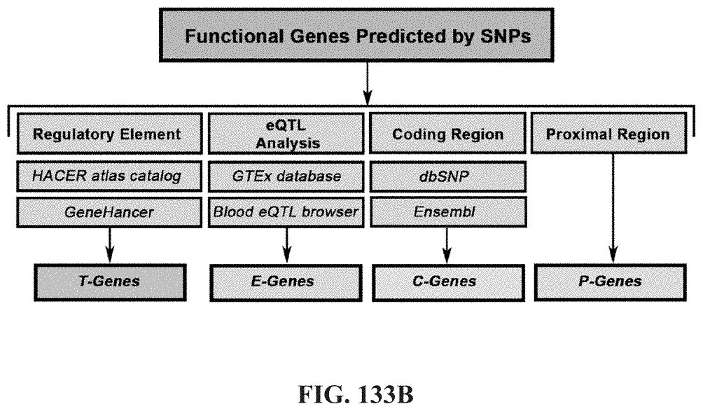

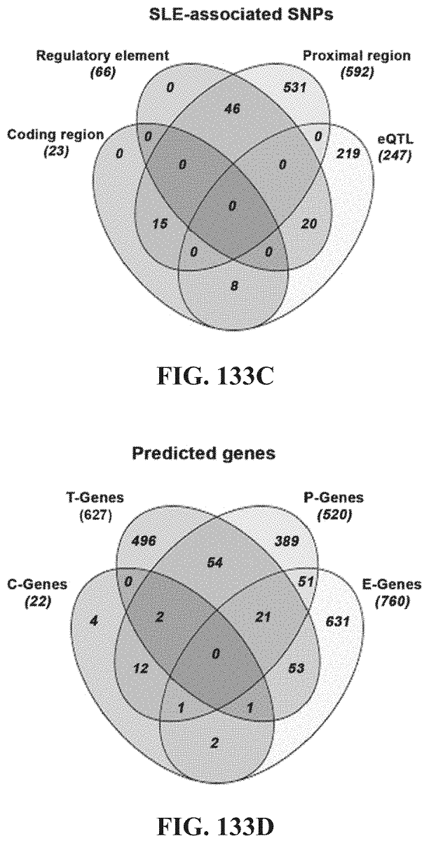

Identification of ancestry-dependent and independent SLE-associated variants and downstream target genes was performed as follows. An extensive transancestral SLE genetic association study using the Immunochip may be performed to identify 839 non-HLA, independent polymorphisms significantly associated with disease (FIG. 133A). To determine how frequently SLE-associated SNPs occur in coding and non-coding regions of the genome, the Ensembl genome browser was used to assess the distribution of genomic functional categories for all Immunochip SNPs (FIG. 133A). Approximately 26% of SNPs mapped to coding (exons, 5′ UTRs, 3′UTRs) or known transcription factor binding regions (TFBS, promoters, enhancers, etc.), whereas the majority of SNPs were found in intronic or intergenic regions exhibiting little evidence of regulatory potential. Furthermore, despite the role of non-coding RNAs in the regulation of gene expression, less than 6% of SNPs mapped to regions containing long non-coding (lnc)RNAs or micro (mi)RNAs.

Since the function of the majority of SNPs was unaccounted for, multiple complementary bioinformatics-based approaches were performed to predict the impact of SLE-associated SNPs on downstream molecular pathways (FIG. 133B). Expression quantitative trait loci (eQTL) analysis can be used to link non-coding risk SNPs with alterations in gene expression, either in cis or trans. eQTL mapping via the GTEx and Blood eQTL browser databases, together with concurrent heterogeneity analysis to determine ancestry, identified 77 EA and 21 AA-specific eQTL linked to 207 and 30 expression genes (E-Genes) unique for EA and AA respectively. A total of 149 eQTLs were common to both ancestries and were linked to 523 shared E-Genes. As expected, the majority of predicted eQTL functioned in cis, consistent with previous studies showing that disease-associated variants typically affect gene expression levels of nearby genes. Furthemore, many eQTL identified here impact E-Gene clusters highly enriched for a common function, suggesting SNPs influencing the expression of multiple genes can help identify potential causal pathways linked to disease phenotypes within individual populations.

FIG. 133A-133D show results obtained by mapping the functional genes predicted by SLE-associated SNPs. FIG. 133A shows a distribution of genomic functional categories for ancestry-specific non-HLA associated SLE SNPs (Tiers 1-3). Non-coding regions include micro (mi)RNAs, long non-coding (lnc)RNAs, introns and intergenic regions. Regulatory regions include transcription factor binding sites (TFBS), promoters, enhancers, repressors, promoter flanking regions and open chromatin. Coding regions were broken down further and include 5′UTRs, 3′UTRs, synonymous and nonsynonymous (missense and nonsense) mutations. FIG. 133B shows that functional genes predicted by SNPs are derived from 4 sources including regulatory elements (T-Genes), eQTL analysis (E-Genes), coding regions (C-Genes) and proximal gene-SNP annotation (P-Genes). FIG. 133C shows a Venn diagram depicting the overlap of all SLE-associated SNPs. FIG. 133D shows a Venn diagram depicting the overlap of and all predicted E-, T-, P-, and C-Genes.

Since variants that alter or disrupt transcription factor binding may also dysregulate gene expression, SNPs were identified within distal and cis regulatory elements (e.g., enhancers and promoters). This analysis included the known regulatory regions identified above, as well as additional ones not previously related to SLE. HACER (Human ACtive Enhancers to interpret Regulatory variants; bioinfo.vanderbilt.edu/AE/HACER/) was used to analyze a catalog of active and in vivo transcribed enhancers that connects regulatory SNPs with target genes (T-Genes). Analysis with HACER identified 41 SNPs overlapping distal regulatory elements (enhancers) predicted to impact the expression of 501 downstream T-Genes. Similar to HACER, GeneHancer links variants in enhancers and promoters with target genes, revealing 25 SNPs linked to 163 T-Genes. These methods identified 472 EA, 9 AA and 143 shared T-Genes.

For variants located in coding regions, 23 SNPs (14 EA, 2 AA, 7 shared) were associated with either non-synonymous amino acid changes or premature termination, affecting 22 genes (C-Genes; 14 EA, 2 AA, and 6 shared). Functional damage scores were determined using SIFT, PolyPhen-2, and PROVEAN which predict the potential impact of amino acid substitutions on protein structure and function. Of the 23 non-synonymous SNPs, 11 were predicted to be deleterious, including the shared SLE risk variant rs2476601 (R620 W) identified to alter the protein tyrosine phosphatase PTPN22, and rs1804182, an identified AA SNP altering the plasminogen activator PLAT.

The remaining 592 SNPs that were not eQTL were assumed to regulate the closest proximal gene (P-Gene), revealing SNP associations with a further 520 P-Genes (465 EA, 34 AA and 21 shared). FIG. 133C depicts the overlap between SNPs based on source, and FIG. 133D shows the overlap between the corresponding predicted E-, T-, C- and P-Genes. No genes were shared among all four groups, and limited commonality was observed between T-, P- and E-Genes, with only 21 genes shared among the three groups. This included genes with known SLE associations (IL12RB1, PXK, BLK, CD44, IRF5, TNPO3, GSDMB, and ORMDL3) and those that have not previously been associated with SLE (ELL, GIMAP8, LRRC25, PLEK, PLTP, PPP26, SF3B1, and SIK2). Despite the overall diversity of genes observed in each list, significant overlap was observed in the number of genes shared between ancestries.

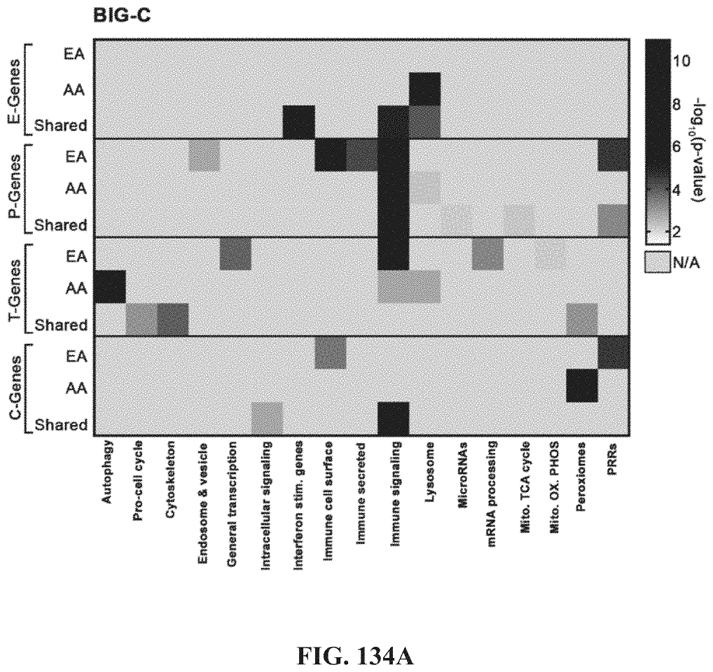

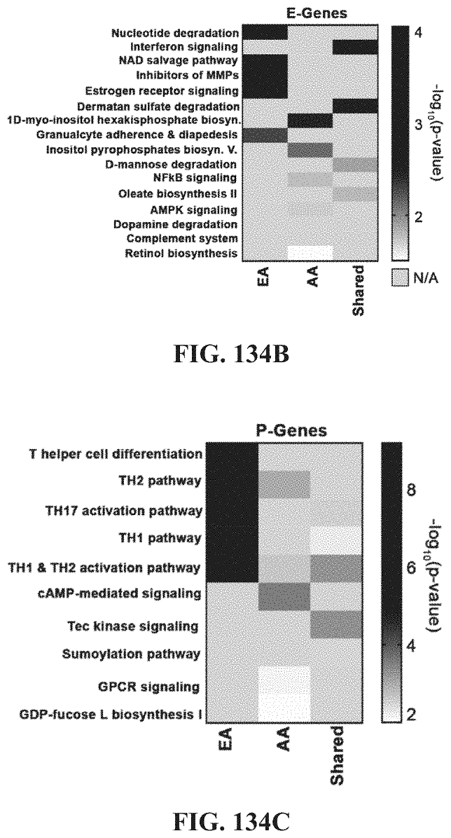

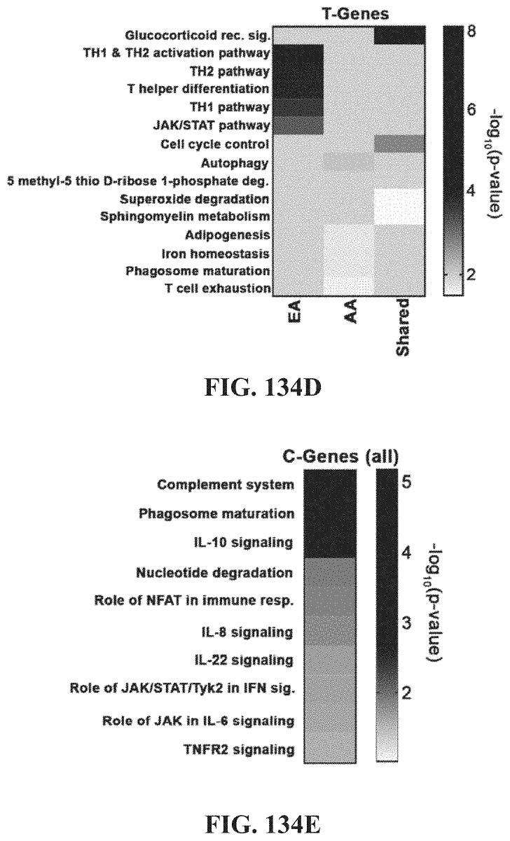

Characterization of gene signatures was performed as follows. Given the heterogeneity of genes identified by eQTL analysis, regulatory element and coding region mapping, as well as traditional annotation based on SNP-gene proximity, a more detailed analysis was performed of the potential functional genomic signatures defining the E-Gene, T-Gene, P-Gene, and C-Gene sets based on ancestry. Gene function was first examined by Biologically Informed Gene Clustering (BIG-C), a functional aggregation tool developed to understand the biological groupings of large gene lists, followed by Ingenuity Pathway Analysis (IPA). Additional analysis of gene function was determined via gene ontology (GO) annotation using the Database for Annotation, Visualization and Integrated Discovery (DAVID). Heatmap visualization of BIG-C category enrichment, IPA canonical pathways and GO terms for each set of genes is shown in FIGS. 134A-134E.

FIGS. 134A-134E show the caracterization of predicted gene signatures. FIG. 134A shows that ancestry-dependent and independent E-, P-, T-, and C-Genes were analyzed to determine enrichment using functional definitions from the BIG-C (Biologically Informed Gene Clustering) annotation library. Enrichment was defined as any category with an odds ratio (OR)>1 and −log 10(p-value)>1.33. FIGS. 134B-134E shows heatmap visualizations of the top five significant IPA canonical pathways for each gene list (E-, P-, T-Genes) organized by ancestry. C-Genes were analyzed together. Top pathways with −log 10(p-value)>1.33 are listed.

Remarkably, functional categorization remained largely consistent within each ancestry despite the derivation of genes from multiple sources. For example, analysis of all EA-associated genes revealed enrichment in processes related to leukocyte and lymphocyte migration and activation. This includes the canonical pathways for agranulocyte adherence and diapedesis and inhibitors of matrix metalloproteinases, as well as the GO term adenylate cyclase activity involved in GPCR signaling pathway (GO:0010578) for E-Genes (FIG. 134B). EA P-Gene pathways included TH1/TH2 activation and multiple GO terms related to response to cytokine (GO: 0034097) (FIG. 134C). Similarly, T-Genes were enriched in JAK/STAT signaling, TH1/TH2 activation pathways and response to cytokine (GO: 0034097) (FIG. 134D). All C-Genes were analyzed together because of the limited number of genes available for analysis, and revealed enrichment in numerous pathways associated with cytokine signaling and immune response activation (FIG. 134E). Receptor-ligand interactions and T cell activation were also reflected in EA BIG-C categories, including immune cell surface, immune secreted, immune signaling, and pattern recognition receptors (PRRs) (FIG. 134A).

For AA-associated genes, E-, P-, and T-Genes were enriched in biological processes related to degradation, including the BIG-C category lysosome, and IPA pathways for autophagy and phagosome maturation (FIG. 134A and FIG. 134D) with additional E-Gene enrichment in peptide cross-linking (GO: 0018149) and keratinocyte differentiation (GO: 0030216). Similar to EA genes, T cell function was also observed in AA, with enrichment in T cell co-stimulation (GO: 00331295) and TH1/TH2 activation pathways for E- and P-Genes respectively (FIG. 134B-134C).

Shared genes were distributed in a diverse range of gene categories. For example, shared E- and T-Genes were enriched in GO terms for keratinization (GO: 0031424), peptide cross-linking (GO: 0018149) and epidermis development (GO: 0008544) similar to AA genes (Supplemental FIG. 2a, c). Phagosome maturation is a pathway common to both AA T-Genes and C-Genes represented by the shared gene ITGAM (FIG. 134C-134D). Shared genes were also involved in processes related to leukocyte cell-cell adhesion (GO: 0007159), cellular activation (GO: 0001775) and the BIG-C category immune signaling, similar to EA genes (FIG. 134A). Furthermore, the T helper signature prevalent in both EA and AA gene sets was also observed in shared genes (FIG. 134C). Finally, shared genes contained a strong core interferon-stimulated gene signature consistent with the role of interferons in the pathogenesis of SLE (FIG. 134A-134B).

Protein interaction-based clustering of predicted genes was performed as follows. The relationship between genes was assessed systematically based on their source regardless of ancestral origin. Protein-protein interaction (PPI) networks consisting of E-, P-, T-, and C-Genes were constructed using STRING (version 10.5), visualized in Cytoscape (version 3.6.1), and clustering for E-, P-, and T-Genes was carried out using the MCODE app plugin to provide an additional level of functional annotation. The resulting networks were further simplified into metastructures defined by the number of genes in each cluster, the number of significant intra-cluster connections predicted by MCODE, and the strength of associations connecting members of different clusters to each other. This dual approach allowed a comparison of the overall topology of different gene clusters while also noting specific interactions between EA, AA, and shared genes.

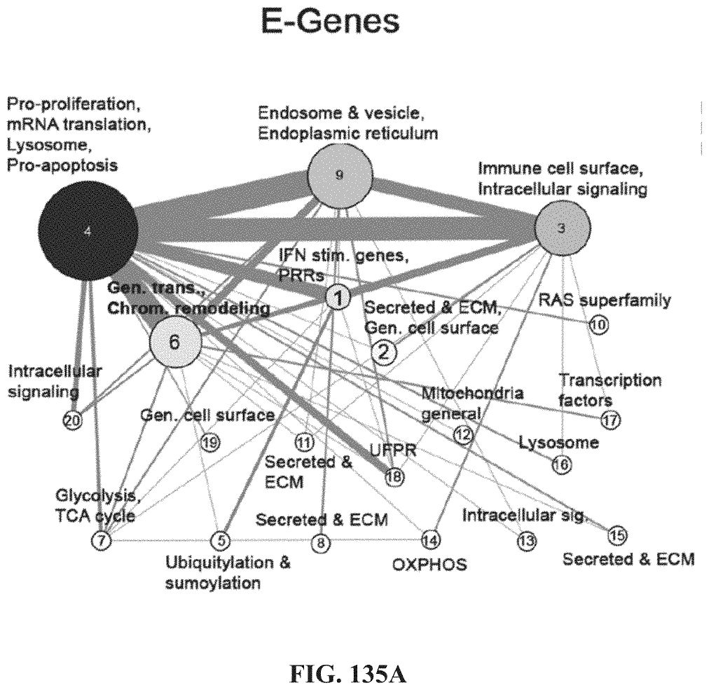

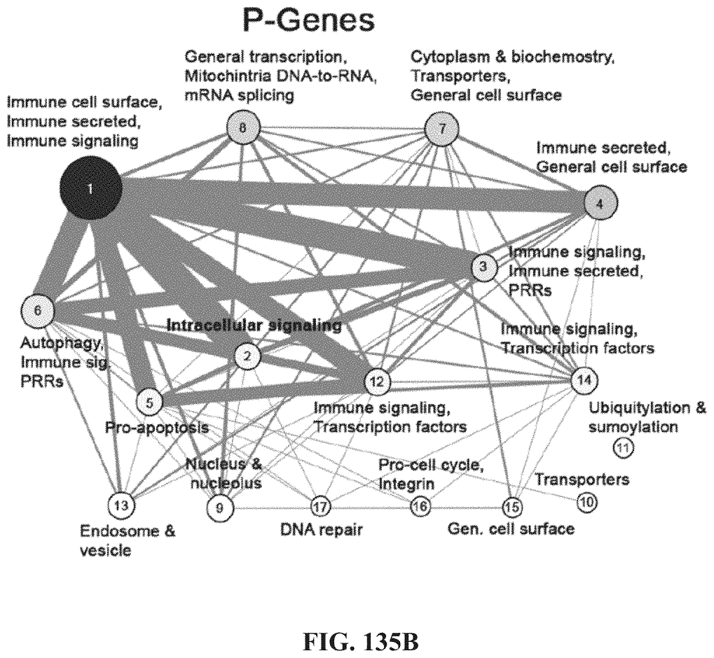

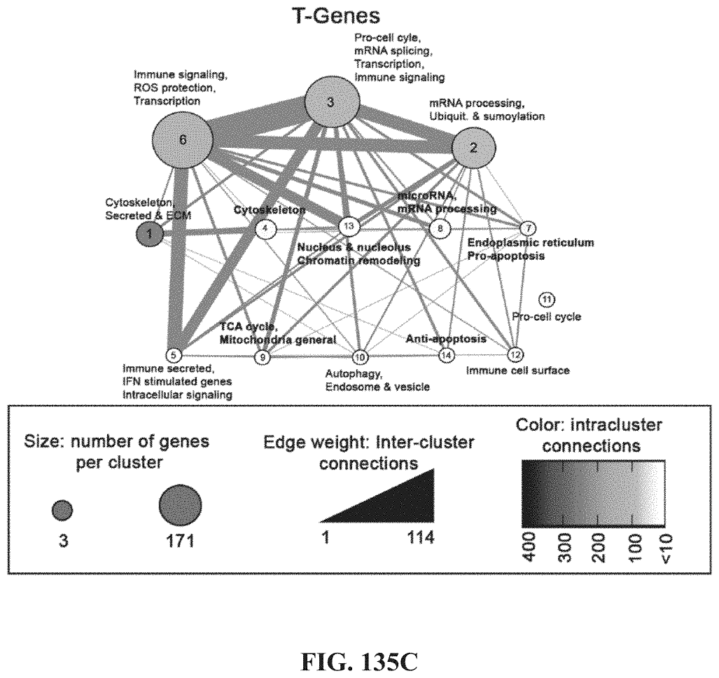

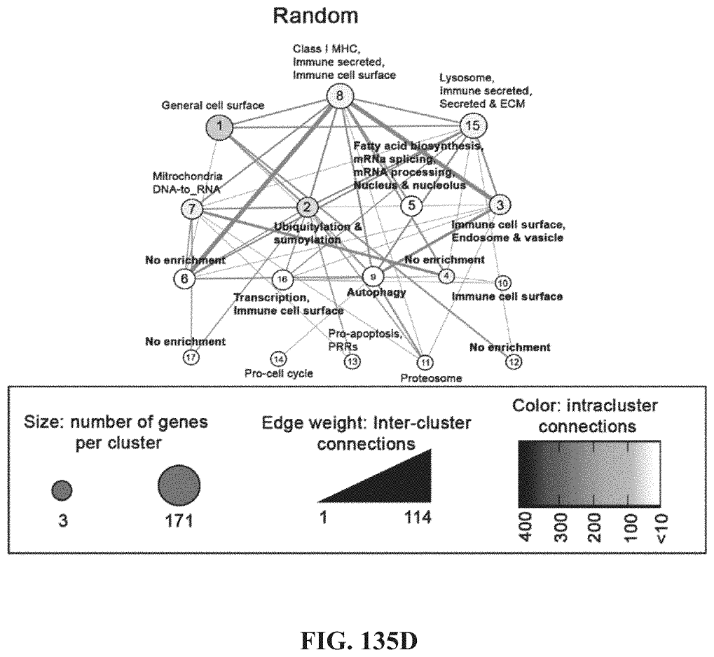

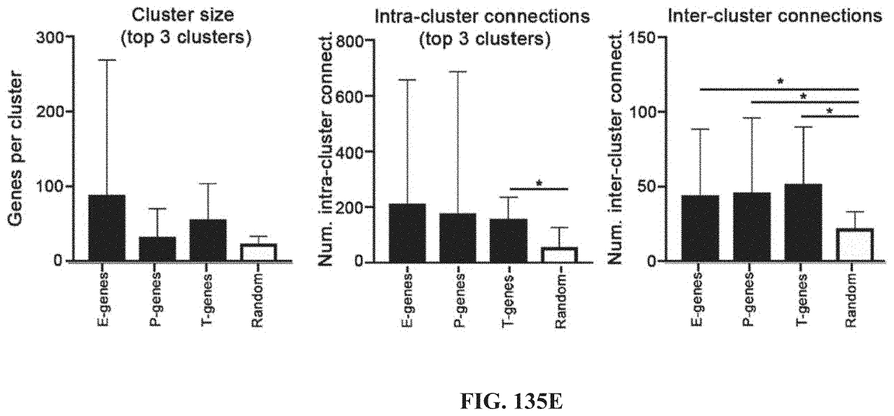

FIGS. 135A-135D show that cluster metastructures were generated based on PPI networks, clustered using MCODE and visualized in CytoScape. Size indicates the number of genes per cluster, edge weight indicates the number of inter-cluster connections and color indicates the number of intra-cluster connections. FIG. 135E shows the quantitation of cluster size, intra- and intercluster connections. Error bars represent the 95% confidence interval; asterisks (*) indicate a p-value<0.05 using Welch's t-test.

E-Gene clusters were dominated by shared E-Genes, with ancestry-specific EA and AA E-Genes distributed throughout the network (FIG. 135A). The largest cluster of E-Genes (cluster 4) was enriched in molecules associated with proliferation, apoptosis, translation and lysosomal degradation. This cluster also contained a number of transcription factors and was highly connected to the immune function enriched cluster 3, as well as clusters 6 and 9 associated with gene regulation and endoplasmic reticulum function, respectively. The robust interferon response evident in the shared E-Gene signature was located in cluster 1, whereas cluster 2 was composed primarily of AA and shared genes involved in keratinocyte function. E-Genes related to metabolic and transcriptional function were also found in clusters 7, 12, 14 and 17. Analysis of cluster topology revealed that SLE associated E-Genes are dominated by aberrantly regulated cell cycle control, transcriptional processes and immune function (FIG. 135A).

Examination of networks constructed of all P-Genes, reveals the predominance of immune function with 7 out of 10 of the largest, intraconnected clusters enriched in immune activity (FIG. 135B). For T-Genes, the largest clusters 6 and 3 were enriched in immune signaling with additional enrichment in transcriptional processes. T-Gene networks were also dominated by BIG-C categories related to mRNA splicing (cluster 3) and RNA processing (clusters 2 and 8). Clusters 1 and 4 were composed almost entirely of shared T-Genes linked to glucocorticoid signaling, including 31 keratin genes derived from rs726848 (FIG. 135C). Although MCODE clustering was not performed on C-Genes because of the small number of genes, more than half of identified C-Genes organized into a STRING network enriched in PRRs, immune signaling and immune cell surface molecules.

To determine whether the predicted genes (E-, T-, or P-Genes) described above represent key genes within relevant SLE biological pathways, a parallel analysis was performed examining PPI networks composed of genes derived from randomly selected Immunochip SNPs. Random SNPs analyzed by eQTL mapping identified a total of 538 random E-Genes, which were used to generate a STRING network and clustered via MCODE (FIG. 135D). Examination of metastructures revealed that random gene clusters exhibited significantly fewer intra-cluster connections and fewer inter-cluster connections, appearing as independent entities lacking robust functional relationships with neighboring clusters (FIG. 135E). Although Immunochip SNPs may be heavily biased toward immunologically relevant genes, the largest, most intraconnected random gene cluster (1) was enriched entirely in general cell surface molecules. Furthermore, composite analysis of all randomly generated E-Genes via BIG-C revealed enrichment in a single category for pro-apoptosis.

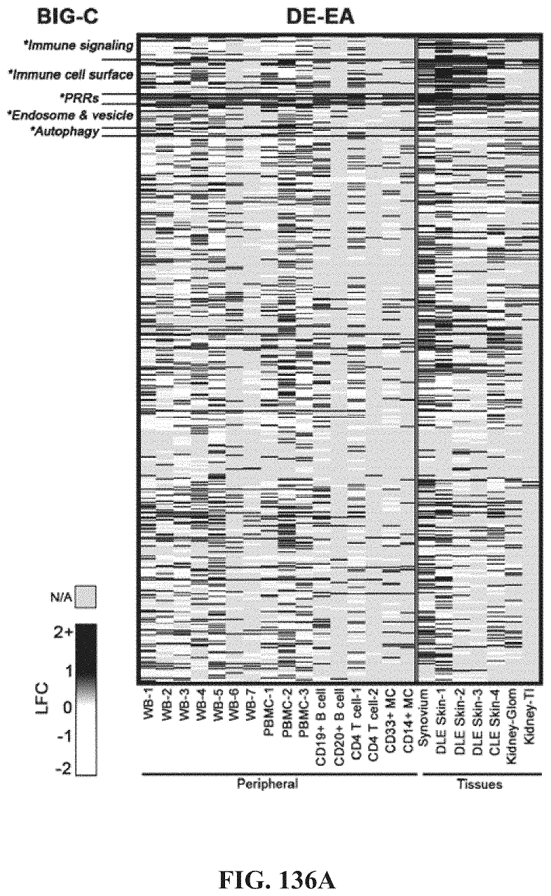

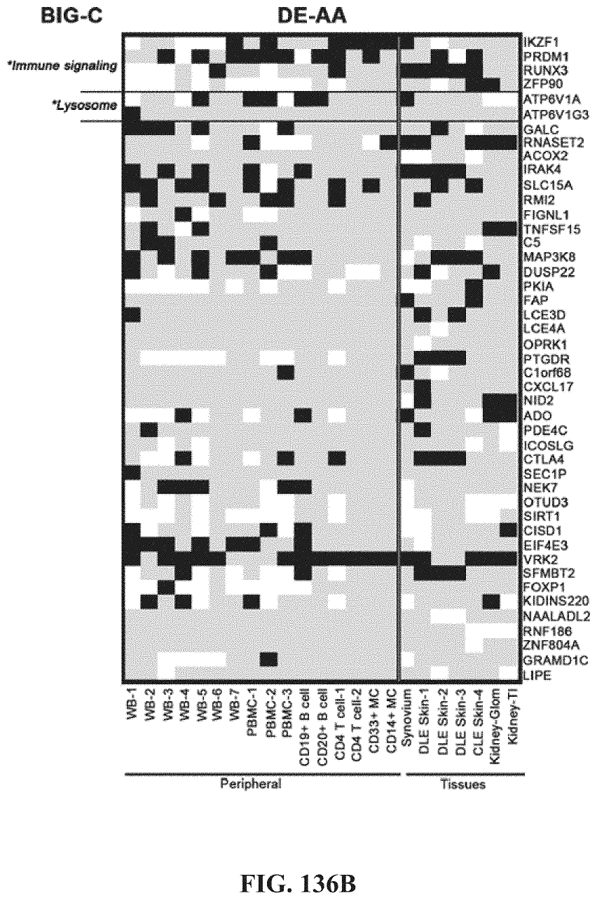

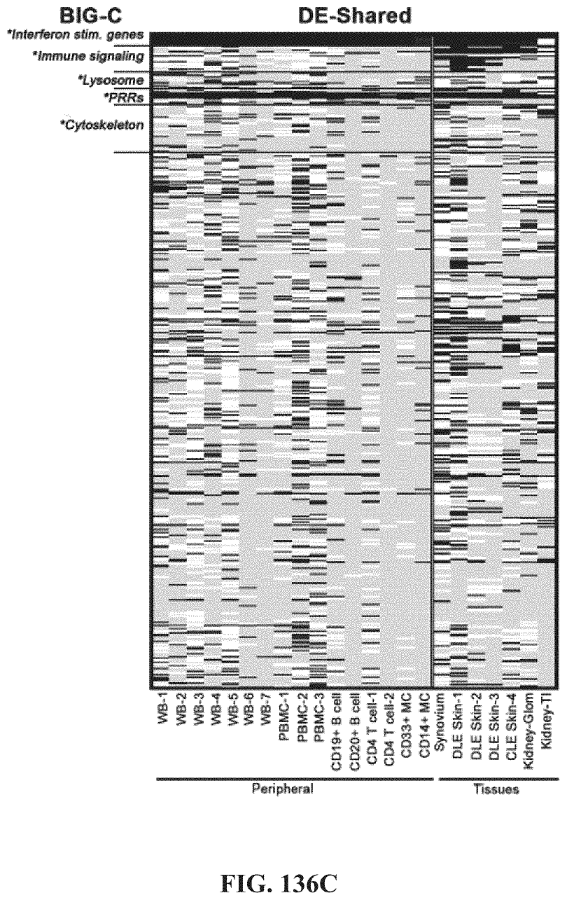

Predicted genes were observed to be linked to altered expression in SLE and were enriched in differential expression datasets as follows. Next, it was determined whether genes linked to specific populations exhibited altered expression in SLE. Ancestry-specific E-, P-, T-, and C-Genes were matched to differential expression (DE) SLE datasets in various tissues, including whole blood, PBMCs, B-cells, T-cells, synovium, skin and kidney (FIG. 136A-136C). Heatmaps depicting the log fold change for each gene were organized based on enriched BIG-C category. 743 differentially expressed EA genes were observed across all datasets enriched for immune signaling, immune cell surface, PRRs, endosome and vesicle and autophagy (FIG. 136A). For AA, 49 genes were differentially expressed exhibiting enrichment in categories related to immune signaling and lysosome. VRK2 and HSPA6 were upregulated in most blood and skin datasets, whereas both IKZF1 and RUNX3 were highly upregulated specifically in skin and synovium datasets (FIG. 136B). Of the genes shared between ancestries, 441 genes were DE, with the interferon stimulated genes (HERC5, IFI35, IFI44L, IFI6, IFIT1, MX1 and SPATSL2L), interferon regulatory factors (IRF4, IRF5 and IRF7) and PRRs (OAS1, OAS2, OAS3, SLC15A4) differentially expressed across all datasets (FIG. 136C). Further, several gene categories were observed that were consistently upregulated in tissue datasets compared to peripheral blood datasets, including genes associated with immune signaling and immune cell surface (FIG. 136C). Overall, the majority of DE predicted genes (regardless of ancestry) were observed in the tissues, including synovium, skin and kidney (FIG. 136A-136C), with fewer DE genes observed in macrophages, T cell and B cell datasets.

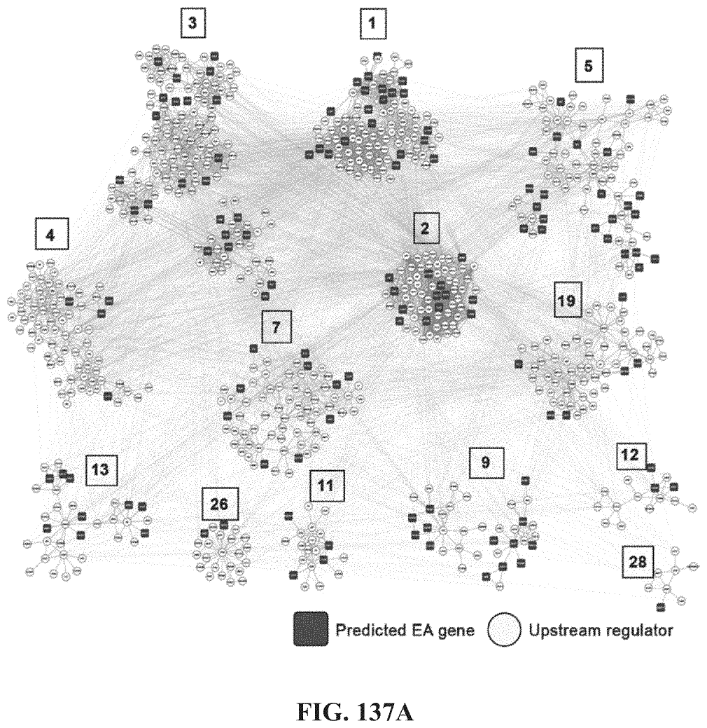

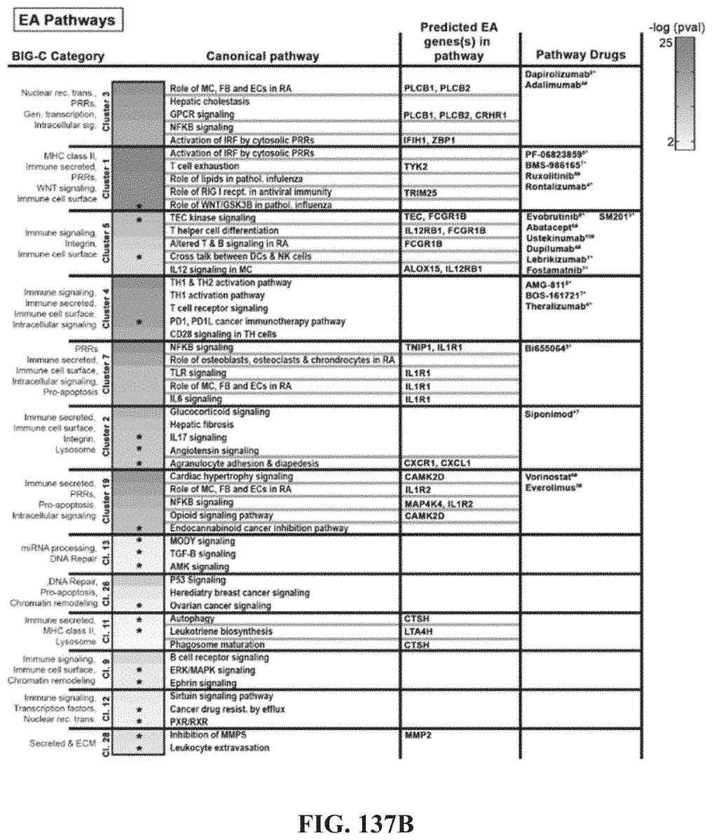

Identification of key signaling pathways was performed as follows. Ancestry-specific key signaling pathways were identified based on differentially expressed genes. To do this, IPA was employed to analyze DE EA, AA and shared gene sets to determine potential biologic upstream regulators (UPRs). Importantly, several of the resulting regulators identified by IPA were also predicted genes, and are known to play major roles in the development of SLE, including IFNG, STAT4, CD40, CTLA4, IRF5 and IRF7. Next, DE predicted genes and UPRs were used as input to build STRING-based PPI networks, visualized in CytoScape, and clustered with MCODE. Individual clusters were then analyzed by BIG-C and IPA to identify those molecules and pathways highly associated with disease. A total of 45 pathways were representative of EA DE genes and UPRs, with the largest clusters 3 and 1 heavily involved in pattern recognition receptor signaling (activation of IRFs by cytosolic PRRs and role of RIG-I in antiviral immunity) (FIG. 137A-137B). Clusters 4 and 5 revealed enrichment in lymphocyte activation and differentiation (TH differentiation pathway, TH1 and TH2 activation pathway, TH1 activation pathway), pathways that were also common to both the AA and shared gene networks. Twenty pathways were unique to EA including several involved in cellular communication (cross talk between dendritic cells and NK cells, leukotriene biosynthesis), cytokine signaling (IL6 signaling and IL17 signaling) and migration (agranulocyte adhesion and diapedesis, leukocyte extravasation, inhibition of MMPs).

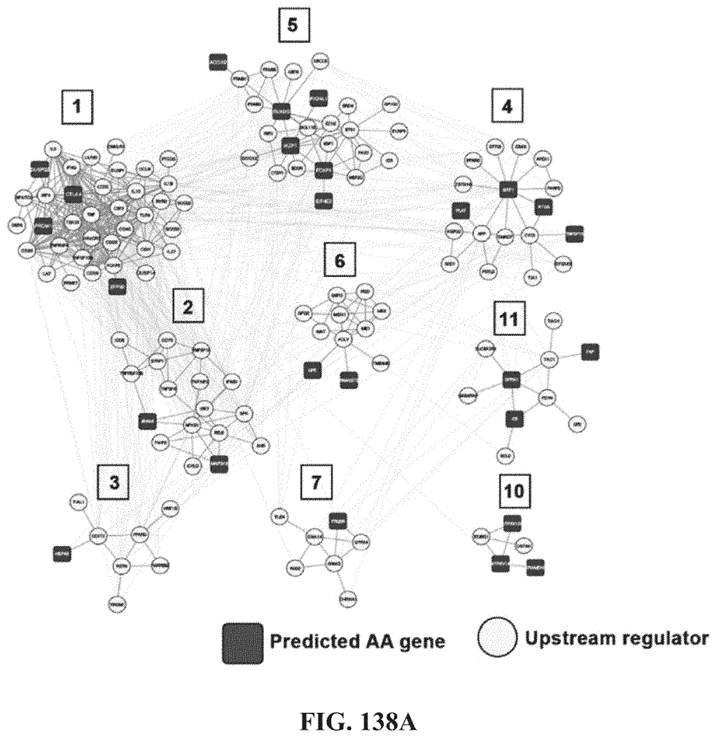

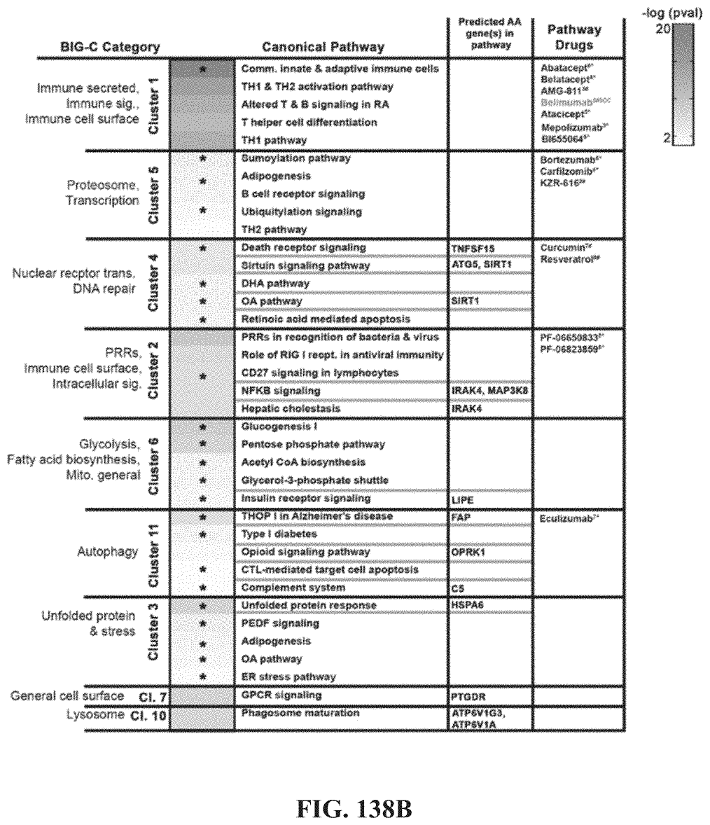

The AA network was smaller (FIG. 138A), containing fewer predicted genes and associated UPRs, yet shared multiple pathways with EA, including B cell receptor signaling, GPCR signaling, opioid signaling, phagocyte maturation and hepatic cholestasis, a pathway involved in bile acid synthesis (FIG. 138B). However, pathways unique to AA were distinct, overwhelmingly represented by processes related to degradation and cellular stress, found in clusters 5, 3 and 11 (sumoylation, ubiquitylation, neuroprotective role of THOPI in Alzheimer's disease, unfolded protein response, ER stress pathway and the osteoarthritis (OA) pathway) as well as metabolic processes in cluster 6 (gluconeogenesis I, pentose phosphate pathway, acetyl CoA biosynthesis, glycerol-3 phosphate shuttle and insulin receptor signaling).

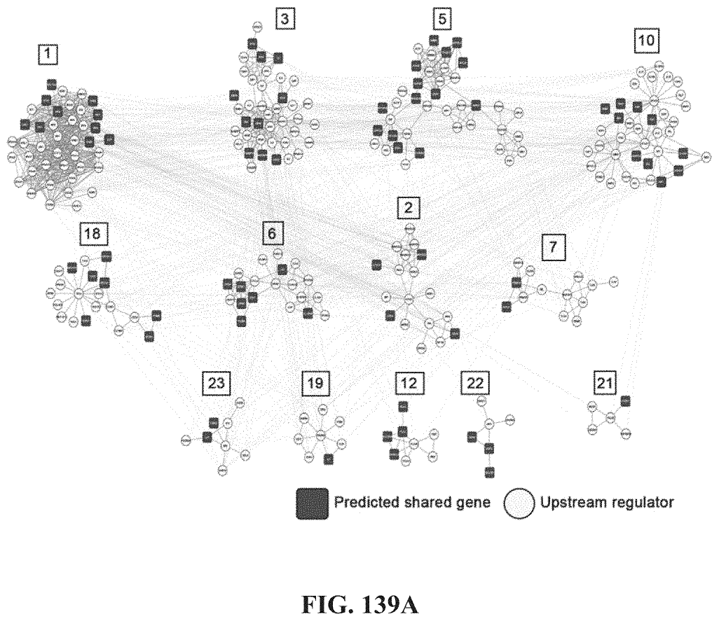

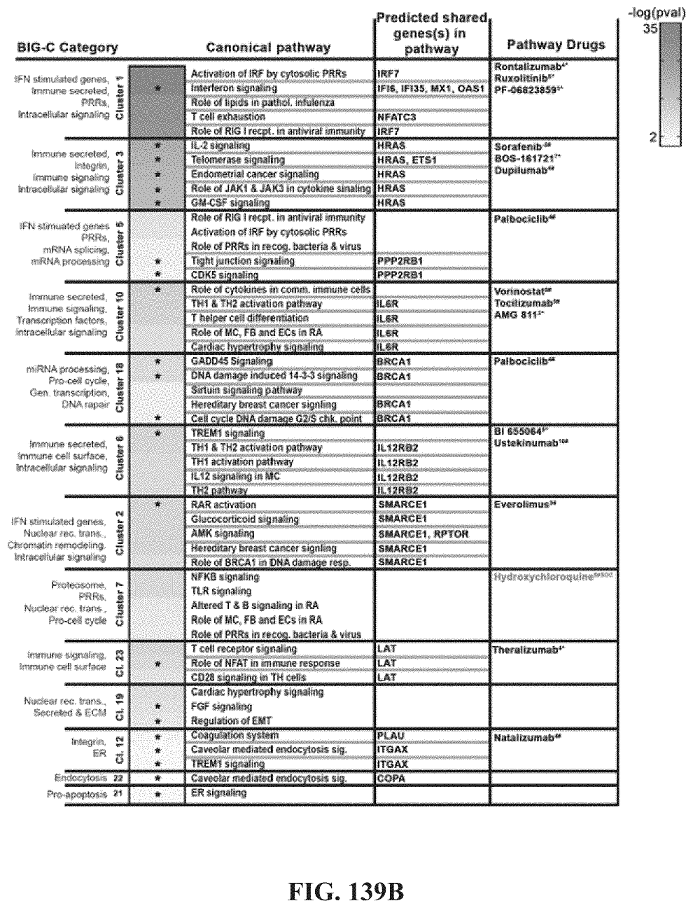

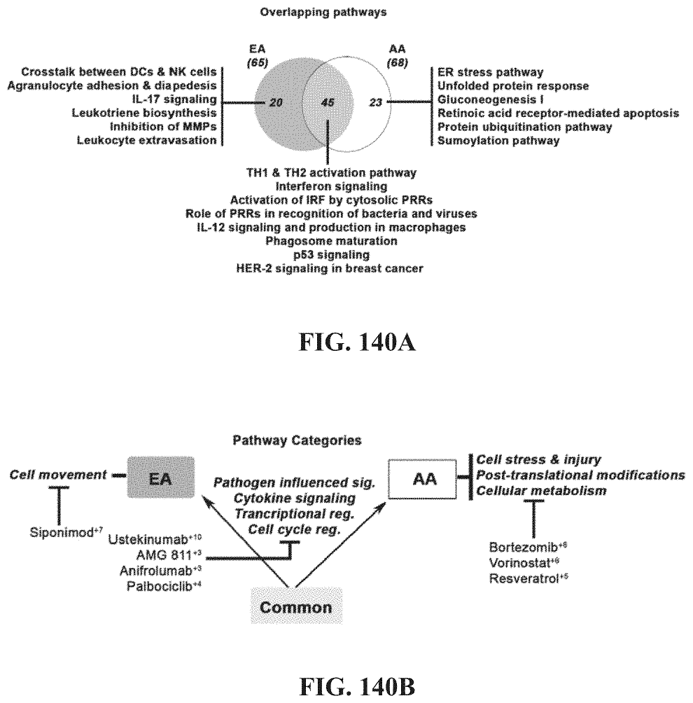

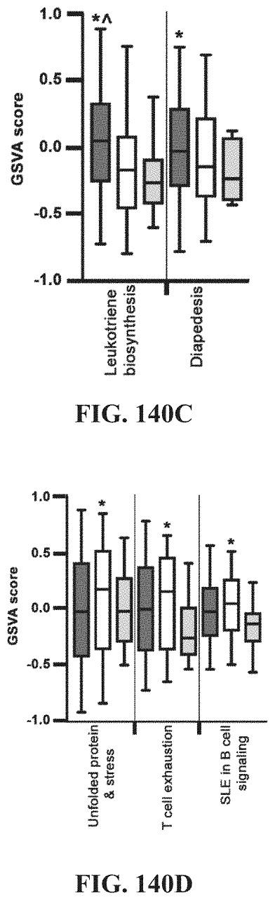

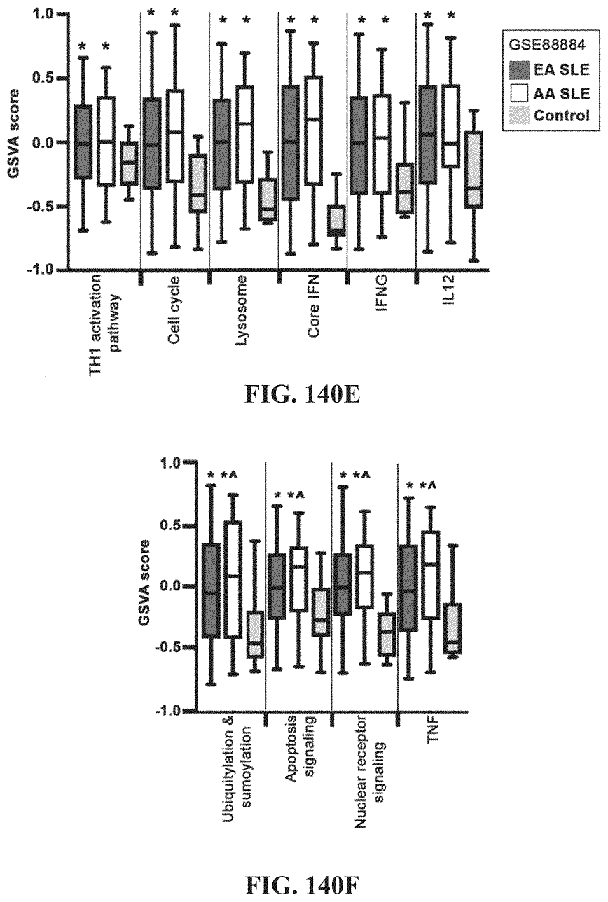

Pathways exemplified by ancestry-independent genes were a blend of both EA and AA pathways. For example, common pathways included IL12 signaling and production by macrophages, TLR signaling and activation of IRFs by cytosolic PRRs, pathways that were predicted by EA genes and UPRs, as well as PRRs in the recognition of bacteria and virus (FIGS. 139A-139B), a pathway shared with AA. FIGS. 140A-140F depicts both the unique and overlapping canonical pathways predicted by the EA and AA gene sets. Examination of pathway categories shared between EA and AA ancestral groups are those commonly associated with SLE representing aberrant immune function, altered transcriptional regulation, and abnormal cell cycle control, providing additional confirmation for the global gene expression analysis presented here (FIG. 140B). Strikingly however, several unique pathway categories were identified that are ancestry-specific, including cell movement for EA, and cell stress and injury, post-translational modification and cellular metabolism for AA.

To validate these pathway predictions, gene set variation analysis (GSVA) was applied to identify differentially enriched gene signatures in SLE patients (EA and AA) and control whole blood (WB). EA and AA predicted genes were used to create a collection of signatures informed by protein-protein interaction networks and IPA canonical pathways, or were previously defined. GSVA enrichment scores using signatures for leukotriene biosynthesis and diapedesis were able to specifically separate EA SLE patients, but not AA patients, from healthy controls (FIG. 140C). Also, it was observed that the leukotriene biosynthesis signature distinguished EA patients from AA patients. In contrast, gene signatures related to cell stress pathways were significantly enriched in AA SLE compared to EA SLE patients for the unfolded protein response (UPR), and AA SLE versus healthy control the for the T cell exhaustion signature (FIG. 140D). AA SLE patients were additionally enriched in the IPA-derived signature for SLE signaling in B cells.

A number of signatures were able to discriminate between SLE patients and controls independent of ancestry, including signatures for TH1 activation pathway, cell cycle and lysosome (FIG. 8e). Cytokine-based signatures for core IFN, IFNG, IL12, and the IFN subtypes IFNA2, IFNB1 and IFNW gene signatures also separated EA and AA SLE patients from controls. Finally, signatures for ubiquitylation and sumoylation, apoptosis signaling, nuclear receptor signaling and TNF were sufficiently discriminatory to separate SLE individuals from controls, and furthermore exhibited significant enrichment in AA patients compared to EA patients (FIG. 140F). Gene signatures for metabolic pathways, including mitochondrial oxidative phosphorylation and glycolysis were also investigated but did not demonstrate any significant change between SLE and control or between ancestries.

Pathway analysis facilitated drug prediction as follows. Pathway identification facilitated drug prediction analysis using a number of available databases, including the Library of Integrated Network Cellular Signatures (LINCS), the Search Tool for Interacting Chemicals (STITCH; version 5.0; stitch.embl.de), as well as IPA, allowing us to identify potential drug candidates for repositioning in SLE. Canonical pathways related to T cell function are shared among ancestries, as are many predicted drugs targeting T cell activity including abatacept, theralizumab and AMG-811 (FIG. 139B). Broader analysis of common pathway categories also indicates the utility of targeting T cell signaling, as well as cytokine pathways such as IL12/23 signaling with ustekinumab and/or interferon signaling with anifrolimab (FIG. 140B). Drugs specific for EA pathways include BMS-986165, a high priority small molecular inhibitor of TYK2 (FIG. 137B), whereas therapeutic candidates targeting AA pathways include the FDA-approved proteasome inhibitor bortezomib, as well as PF-06650833, an IRAK4 specific inhibitor (FIG. 138B). Unique pathway categories identified for EA and AA suggest additional ancestry-specific interventions, such as the small molecule inhibitor of sphingosine-1-phosphate receptor 1 (S1PR1) siponimod for EA (prevents leukocyte egress), and the HDAC inhibitor vorinostat for AA, both of which have shown efficacy in autoimmune clinical trials (FIG. 140B).

SLE may be a chronic autoimmune disease with a strong genetic component. Familial aggregation studies together with GWAS may underscore the contribution of genetics to disease development. Candidate gene studies and GWAS may be performed to identify approximately 90 SLE susceptibility loci. Genetic heterogeneity between ancestral populations may also be important in SLE risk; it may be shown that patients of African descent have a higher prevalence of lupus and experience the disease more severely than those of European ancestry. Despite an improved understanding of how inherited genetic variation impacts disease risk, genetic analyses to date may fail to provide a clear path toward novel therapeutic development. This is of particular concern with respect to AA populations, where the control of disease activity remains suboptimal.

It is important to note that for the vast majority of confirmed SLE risk loci, the causal variant(s) may not have been identified. Potential target genes may be determined based on the strength of associated genetic signal and are therefore taken with inferred functional relevance. Here, a novel strategy was performed using statistical and computational analyses along with data acquired from functional genomic assays and differential gene expression studies to map the global gene expression landscape of SLE and further define the disease-associated pathways responsible for the inherent disparities influencing SLE progression.

Expression quantitative trait loci (eQTL) mapping represents a powerful, bioinformatics-driven methodology to examine the association between specific genetic variations and gene expression levels in tissues. Furthermore, eQTL impacting many genes may be particularly valuable for network modeling and disease analysis. As noted previously, eQTLs influencing the expression of several genes, support the notion that risk haplotypes may harbor multiple functional effects. Here, eQTL analysis identified 207 E-Genes specific for EA, 30 E-Genes for AA, and 523 that were shared across ancestries. While some eQTL mapped to a single causal gene, for example rs4580644 linked to CD38 and rs6131014 linked to CD40, the majority of eQTL SNPs mapped to multiple E-Genes, many of which can be found in the same functional network. This complexity is exemplified by rs4917014, a shared (EA/AA) trans-acting eQTL. Located 5′ of the Ikaros family zinc finger transcription factor IKZF1, the rs4917014*T SLE risk allele is associated with the increased expression of 5 IFN-□ response genes (HERC5, IFI6, IFIT1, MX1 and TNFRSF21) comprising the strong core interferon signature prevalent in the shared E-Gene set.

It may also be shown that disease-susceptibility variants frequently lie in distal regulatory enhancer elements. Indeed, nearly 20% (157) of SNPs analyzed here were located in regulatory regions, including transcription factor biding sites (TFBS), promoters, enhancers, silencers, promoter flanking regions and open chromatin. Using computational gene prediction algorithms that incorporate chromatin interaction data, regulatory SNPs were identified that changed transcription factor binding and were linked to 627 downstream targets (T-Genes). Although some regulatory SNPs also exhibit eQTL effects, we nonetheless uncovered 496 unique T-Genes enriched in a diverse array of functional categories. One major pathway identified was glucocorticoid receptor signaling, a key regulator of epidermal homeostasis, driven by rs726848 at the 17q21.2 locus. This SNP affects multiple intermediate filament keratin T-Genes, as well as the retinoic acid receptor A (RARA), potentially reflecting that fact that skin and joint involvements are among the most common clinical manifestations of SLE. This is further supported by altered expression of E-Genes within and around the late cornified envelope (LCE) locus at 1q21.3 controlling keratinocyte differentiation in both ancestries, including LCE1D, LCE1E, LCE3C, Clorf68, SPRR2G, SPRR2B, SPRR2D, SPRR1B, as well as LCE4A and LCE3D in AA E-Gene sets. Both 17q21 and 1q21-23 may be identified as chromosome regions harboring “hot spots” predisposing to SLE.

Among the loci that lead to changes in gene expression, 23 variants were identified as resulting in non-synonymous amino acid changes affecting 22 genes (C-Genes). Although C-Genes compromise a small proportion of predicted genes overall, several C-Genes, such as the R620 W PTPN22 polymorphism affecting B cell tolerance, may have been linked to SLE and other autoimmune disorders, whereas others may be novel. In the latter case, rs11539148 leads to an amino change (N285I/S) in the glutaminyl-tRNA synthetase QARS, a member of the aminoacyl-tRNA synthetase (ARS) family that plays a major role in cellular homeostasis. B cells typically exhibit high tRNA synthetase expression and increased ARS expression may be linked to a potential role for the ARS in antigen presentation. Not surprisingly, both natural and synthetic tRNA synthetase inhibitors are immunosuppressive, a property that may be exploited in the development of aminoacyl-sulfamide IBI derivatives targeting the proliferative skin disease psoriasis.

Also, traditional locus annotation was employed, mapping the identified risk SNP to the nearest, most proximal gene, resulting in 520 P-Genes (shared among EA and AA). Since computational approaches described herein are predictive, by attempting to provide a more comprehensive translation of GWAS findings, those genes and pathways that are causative and those that represent biological “noise” may be determined. To determine this, PPI networks and clustering based on interaction strength helped exclude those genes lacking strong connections to molecules within or between similarly functioning clusters. Compared to E-, T-, or P-Genes where large, highly connected clusters were observed, randomly generated genes generally formed smaller clusters, exhibited fewer intra- and inter-cluster connections and ultimately appeared as independent entities. Secondly, predicted genes were compared to SLE datasets (SLE vs. control) to determine those genes that were differentially expressed in active disease. To go beyond cataloging disease related molecules, DE genes were used as input into IPA to generate upstream and downstream regulators, which could then be combined for additional network and clustering analysis. This allowed identification of biologically relevant pathways unique to each ancestry, a strategy that revealed essential differences between EA and AA SLE, as well as many pathways that were shared.

Here, pathway-based analysis of predicted genes and their upstream regulators helps clarify the complex polygenic risk associated with SLE. Key dysregulated EA pathways centered around cell movement and cell-cell communication were observed, processes that can be related to many aspects of the disease. This can include, but is not limited to, the migration of leukocytes to sites of inflammation or damage, such as UV exposed skin, and is reflected in pathways for leukocyte extravasation and agranulocyte adhesion and diapedesis, as well as pathways for cell signaling and communication, including leukotriene biosynthesis, IL12 signaling in macrophages, IL17 signaling and cross-talk between DCs and NK cells. Remarkably, gene signatures for leukotriene biosynthesis and diapedesis were sufficiently discriminatory to separate EA SLE patients from controls, providing additional evidence for these pathways in SLE pathogenesis.

In contrast, pathways specific for AA were uniquely enriched in those associated with aberrant degradation, including sumoylation and ubiquitylation, ER stress pathway, unfolded protein response, along with osteoarthritis pathway (cell stress) and the neuroprotective role of THOP1 in Alzheimer's disease, a pathway involved in the presentation of antigen generated by the proteasome. Furthermore, GSVA enrichment scores for cell stress pathways demonstrated unique enrichment in AA SLE patients. The ubiquitin-proteasome system may play a critical role in multiple cellular functions including MHC-mediated antigen processing and presentation, and maintains homeostasis by controlling the breakdown of key proteins involved in cell cycle regulation, transcription and apoptosis. It is therefore not surprising that deregulated ubiquitylation and proteosomal processes may be observed in SLE and several additional inflammatory disorders such as type 1 diabetes, RA and psoriasis. The likely role played by these processes is also reflected in the differential enrichment of these pathways in AA SLE patients compared to both health controls and EA patients.

Given the non-linear, relapse-remitting nature of SLE, the pathways highlighted here for EA and AA may not necessarily define temporal phases of disease progression, nor are they cell-type specific. Rather, the results demonstrate that disparities in SLE may be a consequence of different types of pathways dominating within one ancestral background over another. Other pathways were ancestry independent, as is the case for the interferon signatures prevalent in the shared gene dataset and supported by the GSVA enrichment described here. By focusing on pathways instead of individual genes, this approach identifies “actionable” points of therapeutic intervention with the potential to uniquely impact EA and AA SLE patients. Thus, EA patients may derive particular benefit from treatments that prevent leukocyte or lymphocyte infiltration into tissues. This analysis highlights drugs that modulate, for example, sphingosine-1 phosphate receptor (S1PR), a pleiotropic lipid mediator involved in the regulation of a broad spectrum of cellular functions, including proliferation and survival, cytoskeletal rearrangements, cell motility, and cytoprotective effects. Siponimod, currently FDA approved for the treatment of multiple sclerosis, promotes internalization of S1PR expressed on lymphocytes preventing cell migration to sites of inflammation. Preclinical studies using a first-generation derivative, KRP-203 (fingolimod), may reveal high efficacy in preventing renal damage in lupus-prone mice, due in part, to attenuated T cell infiltration. Given its high Combined Lupus Treatment Score (CoLTS) of +7, siponimod represents a high-priority small molecule drug with potential for repurposing in SLE.

Given the dominance of proteasome and degradation in AA pathways, therapeutic intervention may include proteasome inhibitors like bortezomib (BZ). Interestingly, small-scale safety trials testing the efficacy of BZ may indicate that proteasome inhibition is clinically effective in treating refractory SLE. For example, a (male) AA patient with nephritis (WHO IV) may exhibit a reduction in SLEDAI from 10 to 2 after a single dose of BZ, indicating the possibility that BZ and/or more selective immunoproteasome may hold promise for patients who respond poorly to conventional therapies.

The study demonstrates that multilevel analysis is capable of defining gene regulatory pathways which not only reflect differences in EA and AA populations, but also represent candidate pathways that may be the target of ancestry-specific therapies. Indeed, the ancestral SNP-associated predicted genes and gene expression profiles outlined here illustrate fundamental differences in lupus molecular pathways between ancestries. The results indicate that unique sets of drugs may be particularly effective at treating lupus within each ancestral group.

Identification of SLE-associated SNPs and predicted genes was performed as follows. An SLE Immunochip study identified single nucleotide polymorphisms (SNPs) significantly associated with SLE in AA (2,970 cases; 2,452 controls) and EA (6,748 cases; 11,516 controls) cohorts. SNP proxies (raggr.usc.edu) in linkage disequilibrium (LD) (r2>0.5) with these SLE-associated SNPs were then determined, using the Central European Utah (CEU) population as background for EA SNPs and the Yoruban (YRI) population for AA SNPs. Expression quantitative trait loci (eQTLs) were then identified using GTEx version 6 (GTEXportal.org) and the Blood eQTL browser database (Westra et al) and mapped to their associated eQTL expression genes (E-Genes). In parallel, random E-Gene datasets were generated from randomly selected SLE Immunochip SNPs (Langefeld et al 2017). SNP proxies were then queried by GTEx to generate eQTLs and matched to ENSEMBL gene IDs. To find SNPs in enhancers and promoters, and their associated downstream target genes (T-Genes), the atlas of Human Active Enhancers was queried to interpret Regulatory variants (HACER, bioinfo.vanderbilt.edu/AE/HACER) and the GeneHancer database. To find structural SNPs in protein-coding genes (C-Genes), the human Ensembl genome browser (GRCh38.p12; www.ensembl.org) and dbSNP (www.ncbi.nlm.nih.gov/snp) were queried. Several additional databases were used to generate loss-of-function prediction scores, including SIFT4G (sift-dna.org/sift4g), PolyPhen-2 (genetics.bwh.harvard.edu) and PROVEAN (provean.jcvi.org). All other SNPs were linked to the most proximal gene (P-Gene) or gene region. All predicted genes were divided into an AA, EA, or shared group depending on the ancestral designation of the original SLE-associated SNP.

Genomic functional categories were analyzed as follows. The Variant Effect Predictor tool available on the Ensembl genome browser 93 (www.ensembl.org) was used for annotation information to specify SNPs located within exons, untranslated regions (UTRs), introns, intergenic regions, promoters, enhancers, repressors, promoter flanking regions, open chromatin, micro RNAs, long non-coding RNAs and transcription factor binding sites (TFBS). The online resource tool HaploReg (version 4.1; pubs.broadinstitute.org/mammals/haploreg/haploreg.php) were also used to identify DNA features, regulatory elements and assess regulatory potential.

Differential expression analysis of E-Genes was performed as follows. Predicted genes were compared to multiple differential expression datasets. These datasets include the log fold changes of all genes with significant (FDR<0.2) differential expression in whole blood (WB), peripheral blood mononuclear cells (PBMC), B cells, T cells, myeloid cells, synovium, skin, kidney glomerulus (G), and kidney tubulointerstitium (TI). The FDR was selected a priori to avoid excluding false negatives from the analysis. Cohorts are SLE vs. control (CTL) unless noted otherwise. Additional cohorts include SLE synovium vs. oseteoarthritis (OA) synovium, discoid lupus erythematosus (DLE) skin vs. control skin and subacute cutaneous lupus erythematosus (CLE) skin vs. CTL skin. Datasets include GSE88884 (Illuminate 1 and 2), GSE49454, GSE22908, GSE61635, GSE29536, GSE39088, GSE50772, FDABMC3, EMTAB2713, GSE10325, GSE4588, GSE38351, GSE36700, GSE52471, GSE72535, GSE81071 and GSE32591.

Functional gene set analysis and identification of upstream regulators (UPRs) were performed as follows. For both ancestral groups, predicted gene lists were examined using Biologically Informed Gene Clustering (BIG-C; version 4.4). BIG-C is a custom functional clustering tool developed to annotate the biological meaning of large lists of genes. Genes are sorted into 54 categories based on their most likely biological function and/or cellular localization based on information from multiple online tools and databases including UniProtKB/Swiss-Prot, gene ontology (GO) Terms, MGI database, KEGG pathways, NCBI, PubMed, and the Interactome, and has been previously described (Labonte, Catalina). Enrichment of GO Biological Processes (BP) using the Database for Annotation, Visualization and Integrated Discovery (DAVID) and the Ingenuity Pathway Analysis (IPA; www.qiagenbioinformatics.com) platform provided additional genetic pathway identification. IPA upstream regulator (UPR) analysis was also used to identify potential transcription factors, cytokines, chemokines, etc. that can contribute to the observed gene expression pattern in the input dataset.

Network analysis and visualization were performed as follows. Visualization of protein-protein interaction and relationships between genes within datasets was performed using Cytoscape (version 3.6.1) software. Briefly, STRING (version 1.3.2) generated networks were imported into Cytoscape (version 3.6.1) and partitioned with MCODE via the clusterMaker2 (version 1.2.1) plugin.

Gene set variation analysis (GSVA) was performed as follows. The GSVA (V1.25.0) software package for R/Bioconductor was used. Briefly, GSVA is a non-parametric, unsupervised method for estimating the variation of pre-defined gene sets in patient and control samples of microarray expression datasets. The input for the GSVA algorithm was a gene expression matrix of log 2 microarray of expression values and a collection of pre-defined gene signatures. Enrichment scores (GSVA scores) were calculated non-parametrically using a Kolmogorov-Smirnoff (KS)-like random walk statistic and a negative value for each gene set.

EA and AA predicted genes were used to create GSVA gene signatures. In the case of leukotriene biosynthesis, cell cycle, ubiquitylation and sumoylation, apoptosis signaling and nuclear receptor signaling, genes were initially identified following protein-protein interaction network construction and MCODE clustering. Cluster identity was determined by BIG-C and/or IPA canonical pathway analysis where each cluster was used as a GSVA probe. Gene signatures for diapedesis, TH1 activation pathway, unfolded protein and stress, T cell exhaustion and SLE in B cell signaling were all informed by established IPA canonical pathways. The signature for lysosome was derived from the Lysosome BIG-C category. All interferon and cytokine signatures (core IFN, IFNB1, IFNA2, IFNW, IFNG, IL12 and TNF) have been described previously (catalina). Metabolic signatures for oxidative phosphorylation and glycolysis were based on literature mining and established IPA canonical pathways. Enrichment of each signature was examined in EA and AA SLE patients and healthy control whole blood from GSE 88884. Differences between controls and SLE patient GSVA enrichment scores were determined using the Welch's t-test for unequal variances in PRISM 8.0.

Drug candidate identification and CoLTS scoring were performed as follows. Drug candidates were identified using CLUE, STITCH (version 5.0; stitch.embl.de) and IPA. Each of these tools includes either a programmatic method of matching existing therapeutics to their targets or else is a list of drugs and targets for achieving the same end. In addition to identifying drugs targeting predicted genes directly, these tools were also used to identify drugs targeting select upstream regulators. Where information was available, drugs were assessed by CoLTS to rank potential drug candidates for repositioning in SLE.

### Example 37: GSVA Categories Identified as Associated with Disease Activity in Systemic Lupus Erythematosus (SLE)

Table 71A provides a list of process and signaling pathways and sets of genes associated with disease activity in SLE, which were identified using methods and systems of the present disclosure as being strongly correlative with each given pathway. These sets of genes can be used as effective SLE biomarkers to indicate disease activity via the given pathways.

TABLE 71A
Cellular Process & Signaling

| Pathway | Genes |
|---|---|
| Amino Acid | AASS, OTC, GOT1, AGXT, OAT, GPT2, AGXT2, PSAT1, GLUD1, SDS, PC, |
| Metabolism | GAD1, ARG1, ALDH4A1, ALDH7A1, GRHPR, MCCC2, IVD, OXCT1 |
| Anti inflammation | TNFAIP3, SOCS3, IL1RN, |
| Anti-oxidants | GLRX1, GSTM2, TXN2, PRDX1, PRDX2, PRDX3, PRDX4, PRDX5, PRDX6, |
| | SOD1, SOD2, SOD3 |
| B and T | ISG20, DUSP5, CCND2, RGS1, CAD, HBG2, ISG20, SIT1, SP140 |
| Specific IFN | |
| Cell Cycle | BRCA1, MCM2, NDC80, PTTG1, E2F3, ASPM, AURKA, CCNB2, CCNE1, |
| | CDC20, CENPM, CEP55, GINS2, MCM10, CCNB1, TYMS, NCAPG, |
| | AURKB, MKI67 |
| Cell Specific | IL1RN, LGALS2, SERPING1, SCARB2, STX11, CXCL10, EIF2AK2, PML, |
| CD14 Mono | GADD45B, OAS2, PLSCR1, STAT2, LGALS9, CCL8, TCN2 |
| IFN | |
| Cell Specific | SIGLEC1, IL1RN, LGALS2, SERPING1, SCARB2, STX11, CXCL10, |
| CD14 Mono IFN 2 | EIF2AK2, PML, GADD45B, OAS2, PLSCR1, STAT2, LGALS9, CCL8, TCN2 |
| Cell Specific | IL1RN, LGALS2, STX11, SCARB2, EIF2AK2 |
| CD 14 Mono IFN 3 | |
| Cell Specific | SPIB, STAP1, APOBEC3B |
| CD19 B IFN | |
| Fatty Acid | HACL, HAO. PEX13, PHYH, SLC27A2 |
| Alpha Oxidation | |
| Fatty Acid | ABCD1, ABCD2, ABCD3, ACAA2, ACACB, ACAD11, ACADL, ACADM, |
| Beta Oxidation | ACADS, ACADVL, ACAT1, ACAT2, ACOX1, ACOX2, ACOX3, ACOXL, |
| | ACSBG2, ACSL5, ADIPOQ, AKT2, AUH, BDH2, CPT1A, CPT2, CROT, |
| | DECR1, ECHDC1, ECHDC2, ECHS1, ECI1, ECI2, EHHADH, ETFA, ETFB, |
| | ETFDH, FABP1, GCDH, HADH, HADHA, HADHB, HIBCH, HSD17B4, |
| | IRS1, IRS2, IVD, LEP, PEX2, PEX5, PEX7, SESN2, SLC25A17, SLC27A2, |
| | TWIST1 |
| Fatty Acid | ACAA2, ACACA, ACACB, ACLY, ACSF3, ACSL1, ACSL3, ACSL4, |
| Synthesis | ACSL5, ACSL6, ACSS2, DECR1, ECH1, ECHDC1, ECHDC2, ECHDC3, |
| | FASN, HADH, HADHA, HADHB, MCAT, MECR, MLYCD, OLAH, OXSM, |
| | PC, PECR, SCD, SLC27A3 |
| Gluconeogenesis | ADH4, ADH5, ADH6, ADH7, ADPGK, ADPGK-AS1, AKR1A1, ALDH1A1, |
| | ALDH1A3, ALDH1B1, ALDH2, ALDH3A1, ALDH3B1, ALDH7A1, |
| | ALDH9A1 |
| Glyc, | ALDOB, ALDOC, ALDOA, GPI, PFKL, PFKM, PFKP, PGM1 |
| Gluconeo, PPP | |
| Glycolysis | PFKFB3, PKM, PFKFB2, LDHAL6B, SLC2A1, G6PC2, HKDC1, LDHAL6A, |
| | PFKFB4, SLC2A3, SLC2A4, SLC2A5 |
| Glycolysis and | BPGM, ENO1, ENO2, ENO3, FBP1, FBP2, G6PC, GALM, GAPDH, |
| Gluconeogenesis | GAPDHS, GCK, HK1, HK2, HK3, LDHA, LDHC, PGAM1, PGAM2, PGK1, |
| | PGK2, PKLR, SLC2A2, TPI1, LDHB |
| Short IFN | IFI27, IFI27L1, IFI27L2, IFI44, IFI44L, IFI6, IFIH1, IFIT1, IFIT1B, IFIT2, |
| Signature -2 | IFIT5, IFITM1, IFITM3, ISG15, ISG20, HERC5, HERC6, MX1, MX2 |
| Short IFN | IFI27, IFI44L, EPSTI1, RSAD2, IFI44, CMPK2, SPATS2L, MX1, |
| Signature-1 | EIF2AK2, HERC5, HERC6, IFI6, SAMD9L, SP100, SP110, MX2 |
| High Expression | |
| IFNA2 Signature | ACSL1, ADAR, AGT, AIM2, AKAP2, APOBEC3B, APOBEC3G, APOL3, |
| | ATF3, ATF5, BAG1, BARD1, BCL7B, BLVRA, BRCA1, BRCA2, BST2, |
| | BUB1, C2, CACNA1A, CAD, CAMK2A, CASP1, CASP10, CASP5, CBR1, |
| | CBWD1, CCL13, CCL7, CCL8, CCNA1, CCND2, CD2AP, CD38, CD4, |
| | CD69, CDC42EP1, CDK4, CDKN1A, CFB, CH25H, CHKA, CNTN6, COL3A1, |
| | CTSL, CXCL10, CXCL11, CXCR2, CYP2J2, DAB2, DEFB1, |
| | DLL1, DSC2, DUSP5, DUSP7, DYNLT1, DYSF, ECE1, EDN1, EIF2AK2, |
| | EIF2B1, EIF4ENIF1, ENPP2, EPB41, ETV4, F8, FAF1, FAS, FGF1, FLNA, |
| | FOXO1, FTL, FUT4, GADD45B, GBAP1, GBP1, GBP2, GCH1, GCNT1, |
| | GLB1, GLS, GMPR, GPR161, GUK1, HBG2, HCAR3, HIST2H2AA3, HLA- |
| | DOA, HLA-DRB5, HS6ST1, HSP90AA1, IDO1, IFI16, IFI27, IFI35, IFI44, |
| | IFI44L, IFI6, IFIT1, IFIT5, IFITM1, IFITM2, IFITM3, IFNG, IFRD1, IGL, |
| | IKBKG, IL15, IL15RA, IL1RN, IL6, INPPL1, IRF2, IRF7, ISG15, ISG20, |
| | ITIH2, JAK2, JUP, KCNA3, KDELR2, KIF20B, KLF6, KPNB1, KRT8, LAG3, |
| | LAMP3, LAP3, LEPR, LGALS2, LGALS3BP, LGALS9, LGMN, LMNB1, |
| | LMO2, LY6E, MAP2K5, MCL1, MED1, MGLL, CXCL9, MMP16, MNDA, |
| | MRPS15, MSR1, MX1, MX2, MYD88, NAMPT, NFE2L3, NKTR, NMI, |
| | NR3C1, NUB1, NUPR1, OAS1, OAS2, OAS3, OSBPL1A, PATJ, PDGFB, |
| | PDGFRL, PGGT1B, PKD2, PLSCR1, PMAIP1, PML, PRKRA, PSMB9, |
| | PTCH1, RBCK1, RET, RGS1, RGS6, TRIM34, RPS9, RTP4, SAT1, |
| | SCARB2, SERPING1, SIT1, SLAMF1, SOCS1, SP100, SP110, SP140, SPIB, |
| | ST3GAL5, STAP1, STAT1, STAT2, STX11, SUPT3H, SYN2, TAF5L, TAP1, |
| | TAP2, TARBP1, TCN2, TFDP2, TGM1, TLR3, TLR7, TNFRSF11A, |
| | TNFSF10, TNFSF6, TNK2, TOR1B, TRA2B, TRD, TRIM21, TRIM22, |
| | TRIM26, TRIM38, UBA7, UBE2L6, UBE2S, UBE3A, UNC93B1, USP18, |
| | VAMP5, WARS, WT1, XAF1 |
| IFNB1 Signature | ACLY, ACSL1, ADAM19, ADAP2, ADAR, ADGRE2, ADM, AFF3, AGT, |
| | AIM2, AKAP10, AKAP2, ALOX12, ALOX5, ANXA4, APOBEC3B, |
| | APOBEC3G, APOL3, ATF3, ATF5, ATM, ATP13A1, B4GAT1, BAG1, |
| | BAK1, BARD1, BCL11A, BCL7B, BGN, BLNK, BLVRA, BLZF1, BRCA1, |
| | BRCA2, BST2, BUB1, C3AR1, CACNA1A, CAD, CALD1, CAMK2A, |
| | CAPN2, CASP1, CASP10, CASP5, CBR1, CBWD1, CCL13, CCL3L1, CCL4, |
| | CCL7, CCL8, CCNA1, CCND2, CCR1, CCR5, CCRL2, CD163, CD164, |
| | CD2AP, CD38, CD4, CD59, CD69, CD72, CD86, CDK17, CDKN1A, CENPA, |
| | CENPE, CFB, CFLAR, CH25H, CHI3L2, CHKA, CISH, CKB, CMAHP, |
| | CNTN6, CNTRL, COL3A1, COX17, CSF2RB, CTSL, CXCL10, CXCL11, |
| | CXCL2, CXCR2, CYBB, CYP19A1, CYP2J2, DAB2, DEFA1, DEFB1, |
| | DHFR, DLL1, DMXL1, DNMT1, DRAP1, DSC2, DUSP5, DUSP7, DYNLT1, |
| | DYSF, E2F1, ECE1, EDN1, EGR1, EIF2AK2, EIF2B1, EIF4ENIF1, ELF1, |
| | ELF4, ENPP2, EPB41, ETV4, ETV6, F8, FAF1, FAS, FBXW2, FCGR1A, |
| | FCMR, FGF1, FLNA, FMR1, FOXO1, FPR2, FTL, FUT4, GADD45B, |
| | GBAP1, GBP1, GBP2, GCH1, GCNT1, GLS, GMPR, GPI, GPR161, GUK1, |
| | HBG2, HCAR3, HHEX, HIST2H2AA3, HK2, HLA-DOA, HS6ST1, |
| | HSP90AA1, HSPA1A, HSPA1L, IDO1, IFI16, IFI27, IFI35, IFI44, IFI6, IFIT1, |
| | IFIT5, IFITM1, IFITM2, IFITM3, IFNG, IFRD1, IGL, IKBKE, IKBKG, IL15, |
| | IL15RA, IL18BP, IL18R1, IL1RN, IL6, IL7, INPP5D, INPPL1, IRF1, IRF2, |
| | IRF4, IRF7, IRF9, ISG15, ISG20, ITGAL, ITGAX, JAK2, JCHAIN, JUP, |
| | KCNA3, KCNMB1, KDELR2, KIF20B, KLF2, KLF6, KLRB1, KPNB1, |
| | KRT8, LAG3, LAMP3, LANCL1, LAP3, LBR, LEPR, LGALS2, LGALS3BP, |
| | LGALS9, LGMN, LILRA1, LINC00597, LMNB1, LMO2, LTA, LTB4R, |
| | LY6E, LYN, MAP2K5, MAP3K8, MARCKS, MBNL, MCL1, MED1, MEF2A, |
| | MFHAS1, MGLL, CXCL9, MNDA, MRPS15, MS4A7, MSR1, MX1, MX2, |
| | MYD88, NAMPT, NAPSA, NBN, NCF1, NCOA2, NEBL, NEK4, NFE2L3, |
| | NKTR, NMI, NOTCH1, NR3C1, NR4A3, NUB1, NUPR1, OAS1, OAS2, |
| | OAS3, PATJ, PAX5, PAX8, PDE4B, PDGFB, PDGFRL, PFKFB3, PFKP, |
| | PIM2, PKD2, PLEK, PLSCR1, PMAIP1, PML, PMS2, PPP2R2A, PRKAG1, |
| | PRKRA, PRKX, PSMB8, PSMB9, PTCH1, PTGER2, RALB, RASGRP1, |
| | RBBP6, RBCK1, RERE, RGS1, RGS6, RIN1, RIPK1, RIPK3, RIPOR2, |
| | RNF114, TRIM34, RPS6KA5, RPS9, RRBP1, RTP4, SAT1, SCARB2, SDS, |
| | SELL, SERPIND1, SERPING1, SFTPB, SIDT2, SIT1, SLAMF1, SMO, SNX2, |
| | SOCS1, SOS1, SP100, SP110, SP140, SPIB, SPTA1, SPTLC2, SRRM2, SSB, |
| | ST3GAL5, STAP1, STAT1, STAT2, STOML2, STX11, SUPT3H, TANK, |
| | TAP1, TAP2, TAPBP, TARBP1, TBX21, TCN2, TFDP2, TFF1, TGM1, THY1, |
| | TLR1, TLR3, TLR7, TNFAIP2, TNFRSF11A, TNFSF10, TNFSF6, TNK2, |
| | TOR1B, TRA2B, TRD, TRG, TRIM21, TRIM22, TRIM26, TRIM38, |
| | TSPAN15, TXK, UBA7, UBE2L6, UBE2S, UBE3A, UBQLN2, UNC93B1, |
| | USP15, USP18, USP25, USPL1, UVRAG, VAMP5, WARS, WIPF1, WT1, |
| | XAF1, ZNF107 |
| IFNG Signature | ACLY, ACSL1, AFF2, AIM2, AKAP10, APOL3, ATF3, ATM, C1QB, C4A, |
| | CALD1, CASP1, CASP10, CCL8, CCND2, CCR5, CD38, CDKN1A, CFB, |
| | CKB, CLEC10A, CPT1B, CSF2RB, CTNND2, CXCL10, CXCL11, CYBB, |
| | EDN1, EPB41, ETAA1, ETV4, F8, FAS, FBLN1, FBXL2, FCGR1A, FLII, |
| | GADD45B, GBP1, GBP2, GCH1, GCNT1, GLS, GSTM5, HBG2, HHEX, HP, |
| | ICAM1, IDO1, IFI27, IFI44, IL15, IL15RA, IL18BP, IL1A, IL7, IRF1, IRF8, |
| | JAK2, JCHAIN, KLF2, LAP3, LIMK2, LMNB1, CXCL9, MMP25, MRPS15, |
| | MSR1, NET1, NIN, NKTR, NLRP1, NR3C1, OAS1, OAS3, P2RY13, PCDH9, |
| | PLA2G4C, PLEK, POLR2B, PSMB9, PTCH1, RALB, RGS1, SERPIND1, |
| | SERPING1, SFTPB, SLAMF1, SLC1A5, SOCS1, SP100, SPRY4, SRRM2, |
| | STAT1, STAT2, STX11, TAP1, TAP2, TBX21, TENM1, TFF1, TNFAIP2, |
| | TNFSF10, UBD, UBE2C, UBE2L6, UBE3A, VAMP5, VSNL1, WARS, XRN1 |
| IFNK unique | IFNK, MMP13, C1orf141, TMEM140, GBP5, RBM11, CLEC7A, MBL1P, |
| | GIMAP2, CEMIP, TRANK1, SIDT1, RASGRP3, MAK, PLEKHA4, ZFP42, |
| | NLRC5, SLC15A3, PLA2G4E, UBA7, SLC16A12, SLC13A5, LOC100130093, |
| | TMEM229B, LOC100507463, HELZ2, RBM43, FRMPD1, BEST3, FAM46C, |
| | STARD5, NCOA7, PROX1, PARP14, TAGAP, LOC153684, DDX60L, |
| | B4GALNT2, PPARGC1A, MASTL, ZNF608, CACNB4, C5orf56, CD274, |
| | SLC25A36, RNF122, ANKRD22, BBC3, HDX, ZNF107, ACE2, FAM90A1, |
| | FER1L6, SYNPO2, SLC25A28, APOBEC3F, KIAA1239, KCNB2, HRASLS2, |
| | TRIML2, C21orf91, PPIF, CBR3, CARD16, IL22RA1, WFDC5, CENPT, |
| | SLC28A3, BTN3A3, BMP4, MALT1, SECTM1, TREX1, HCP5, CASP7, |
| | RUNX2, B3GNT7, TRPM6, FBXO6, SP140L, PRDM8, LHFPL2, ANTXR2, |
| | TNF, BCL2L13, HSPG2, HLA-F, BTN3A1, ZHX2, TRIM25, RHPN1-AS1, |
| | KBTBD8, TMEM27, SLFN5, TCF4, PRKD2, KIAA0040, HLA-B, OGFR |
| IFNW1 Signature | ABCB10, ACLY, ACSL1, ADAR, ADM, AGT, AIM2, AKAP10, AKAP2, |
| | ALOX12, ANXA4, APOBEC3B, APOBEC3G, APOL3, ATF3, ATF5, ATM, |
| | B4GAT1, BAG1, BARD1, BCL11A, BCL7B, BLVRA, BLZF1, BRCA1, |
| | BRCA2, BRD4, BST2, C3AR1, CAD, CALD1, CAMK2A, CAPN2, CASK, |
| | CASP1, CASP10, CASP5, CBR1, CBWD1, CCL13, CCL3L1, CCL7, CCL8, |
| | CCNA1, CCND2, CCR1, CCR5, CCR7, CCRL2, CD164, CD2AP, CD38, CD4, |
| | CD47, CD59, CD69, CDKN1A, CENPE, CFB, CFLAR, CHKA, CKB, |
| | CMAHP, CNTN6, CNTRL, COL3A1, CSF2RB, CTSL, CXCL10, CXCL11, |
| | CXCR2, CYBB, CYP19A1, CYP2J2, DEFB1, DLL1, DSC2, DUSP5, DUSP7, |
| | DYNLT1, DYSF, E2F1, ECE1, EDN1, EGR1, EIF2AK2, EIF2B1, EIF4ENIF1, |
| | ENPP2, EPB41, ERCC4, ETV4, ETV6, F8, FAF1, FAS, FCER1G, FGF1, |
| | FGF13, FGL2, FLNA, FMR1, FOXO1, FTL, FUT4, GADD45B, GBAP1, |
| | GBP1, GBP2, GCH1, GCNT1, GLB1, GLS, GMPR, GPR161, GSTM5, GUK1, |
| | HBG2, HHEX, HIST2H2AA3, HLA-DOA, HS6ST1, HSP90AA1, HSPA1A, |
| | IDO1, IFI16, IFI27, IFI35, IFI44, IFI6, IFIT1, IFIT5, IFITM1, IFITM2, |
| | IFITM3, IFRD1, IGL, IKBKG, IL15, IL15RA, IL18R1, IL1RN, IL6, IL7, INPPL1, |
| | IRF1, IRF2, IRF7, IRF8, ISG15, ISG20, ITIH2, JAK2, JCHAIN, JUP, |
| | KCNA3, KDELR2, KIF20B, KLF6, KPNB1, KRT8, LAG3, LAMP3, LAP3, |
| | LEPR, LGALS2, LGALS3BP, LGALS9, LGMN, LINC00597, LMNB1, |
| | LMO2, LY6E, LYN, MAP2K5, MARCKS, MBNL1, MCL1, MED1, MEF2A, |
| | MGLL, CXCL9, MLF1, MMP16, MNDA, MRPS15, MS4A7, MSR1, MX1, |
| | MX2, MYD88, NAMPT, NCF1, NFE2L3, NKTR, NMI, NPTX1, NR3C1, |
| | NUB1, NUPR1, OAS1, OAS2, OAS3, OSBPL1A, PATJ, PAX8, PDGFB, |
| | PDGFRL, PKD2, PLEK, PLSCR1, PMAIP1, PML, PPP2R2A, PRKAG1, |
| | PRKRA, PSMB9, PTCH1, PTGER2, RALB, RBBP6, RBCK1, RERE, RGS1, |
| | RGS6, TRIM34, RPS6KA5, RTP4, SAT1, SCARB2, SDS, SELL, SERPIND1, |
| | SERPING1, SFT2D2, SIT1, SLC30A4, SOCS1, SOS1, SP100, SP110, SP140, |
| | SPIB, SRRM2, ST3GAL5, STAP1, STAT1, STAT2, STX11, SUPT3H, TAP1, |
| | TAP2, TARBP1, TBX21, TCN2, TFDP2, TFF1, TGM1, THY1, TLR3, TLR7, |
| | TNFAIP3, TNFRSF11A, TNFSF10, TNFSF6, TNK2, TOR1B, TRA2B, TRD, |
| | TRIM21, TRIM22, TRIM38, UBA7, UBE2C, UBE2L6, UBE2S, UNC93B1, |
| | USP18, USP25, WARS, WIPF1, WT1, XAF1, ZNF107 |
| IL1 cytokines | IL1B, IL18 |
| IL12 Signature | ACLY, AKAP10, APOL3, BACH2, BRCA2, CALD1, CASK, CASP1, CCR5, |
| | CDKN3, CXCL10, CXCR3, CYBB, DEFA1, ETAA1, FASLG, FBXL2, |
| | FCER2, FCGR1A, GBP1, GBP2, GLS, GNPDA1, GSTM5, GZMB, HHEX, |
| | HP, HSPA6, IFNG, IL16, IL18BP, IL18R1, IL1A, INPP5D, INSIG1, IRF1, |
| | KLF2, KRT8, LIMK1, LINC00597, LY75, MMP25, NIN, NLRP1, PCDH9, |
| | SELL, SERPIND1, SLAMF1, SOCS1, STAT1, TAP2, TBX21, TFF1, |
| | TNFAIP2, TNFAIP3, TNFSF10, TXK |
| IL21 Signaling | ROR2, DGKG, GIPR, NKG7, CASC1, NDRG4, ST6GALNAC2, ABCC8, |
| | FAT2, SEMA7A, CYP27A1, SLC7A5, DNASE1L2, SLC12A8, RPP25, MUC1, |
| | AIM2, MYOZ3, EHF, HEG1, PRR4, FOXD2, ABCA6, ABCB11, KCNC3, |
| | BOK, IQCG, CFD, ALDH4A1, PPAP2C, HABP4, TF, SPARCL1, USP2, |
| | OPRL1, BAIAP3, ELOVL4, FA2H, LAG3, FOSB, LILRB4, CLSTN3, |
| | TBC1D19 |
| Inflammasome | AIM2, CASP1, CASP5, CTSB, NAIP, NLRC4, NLRP3, NOD2, PYCARD, |
| | P2RX7, NEK7, NLRP1, PANX1, GSDMD, GSDMB, RIPK1 |
| Inflammatory | C1QA, C1QB, C1QC, C1RL, CCL2, CCL8, CXCL1, CXCL2, CXCL10, GRN, |
| Secrete | IK, IL18RAP, IL1B, IL1RN, S100A8, THBD, TNF |
| Low Disease | SNORA23, SNORA38B, SNORA73B, SNORA11, RNU4ATAC, SNORA73A, |
| Down Signature | SNORA16A |
| Low Disease | FCGR1A, SNORD80, SNORD44, SNORD47, SNORD24, CEACAM1, |
| UP Signature | LGALS1 |
| MHC Class II-1 | HLA-DPB2, HLA-DQA2, HLA-DPA1, HLA-DOA, HLA-DOB, CIITA, CD74, |
| | HLA-DRB1, HLA-DMB |
| MHC Class II-2 | HLA-DMA, HLA-DMB, HLA-DPA1, HLA-DPB1, HLA-DPB2, HLA-DQA1, |
| | HLA-DQA2, HLA-DQB1, HLA-DQB2, HLA-DRA, HLA-DRB1, HLA-DRB3, |
| | HLA-DRB4, HLA-DRB5, HLA-DRB6 |
| mRNA translation | RPS3A, RPL5, EIF3L, EEF1A1, EEF2, EEF1B2, RPS23, BTF3, RPL4, RPL3, |
| | RPL7A, RPL10A, RPL30, RPS8, EIF3F, RPS16, RPS3, RPS2, NACA, EIF2D, |
| | RPL14 |
| Neg Reg PRR | SOCS1, USP18, NMI, RBCK1, TRIM21, RNF125, IFI35, GBP4 |
| Signaling | |
| Oxidative | ATP5A1, ATP5B, ATP5D, ATP5E, ATP5F1, ATP5G1, ATP5G2, ATP5G3, |
| Phosphorylation | ATP5H, ATP5I, ATP5J, ATP5J2, ATP5L, ATP5O, ATP5S, ATP6, ATP8, |
| | BCS1L, CEP89, COA1, COA3, COA4, COA5, COA6, COA7, COX10, |
| | COX10-AS1, COX11, COX14, COX14, COX15, COX16, COX17, COX18, |
| | COX19, COX20, COX4I1, COX4I2, COX5A, COX5B, COX6A1, COX6A2, |
| | COX6B1, COX6B2, COX6C, COX7A1, COX7A2, COX7A2L, COX7B, |
| | COX7B2, COX7C, COX8A, COX8C, CYC1, CYCS, DNAJC15, MT-CO1, |
| | MT-CO2, MT-CO3, MT-CYB, MT-ND1, MT-ND2, MT-ND4, MT-ND6, ND1, |
| | ND2, ND3, ND4, ND4L, ND5, ND6, NDUFA1, NDUFA10, NDUFA11, |
| | NDUFA12, NDUFA13, NDUFA2, NDUFA3, NDUFA4, NDUFA4L2, |
| | NDUFA5, NDUFA6, NDUFA7, NDUFA8, NDUFA9, NDUFAB1, NDUFAF1, |
| | NDUFAF2, NDUFAF3, NDUFAF3, NDUFAF4, NDUFAF4, NDUFAF5, |
| | NDUFAF6, NDUFAF6, NDUFAF7, NDUFAF8, NDUFB1, NDUFB10, |
| | NDUFB11, NDUFB2, NDUFB2-AS1, NDUFB3, NDUFB4, NDUFB5, |
| | NDUFB6, NDUFB7, NDUFB8, NDUFB9, NDUFC1, NDUFC2, NDUFS1, |
| | NDUFS2, NDUFS3, NDUFS4, NDUFS5, NDUFS6, NDUFS7, NDUFS8, |
| | NDUFV1, NDUFV2, NDUFV3, NUBPL, OXA1L, RFESD, SCO1, SCO2, |
| | SLC25A4, SURF1, TACO1, TIMMDC1, TMEM126B, TRAP1, TTC19, |
| | UQCC1, UQCC2, UQCC3, UQCR10, UQCR11, UQCRB, UQCRC1, |
| | UQCRC2, UQCRFS1, UQCRH, UQCRHL, UQCRQ |
| Pentose | G6PD, H6PD, PGD, PRPS1, PRPS1L1, PRPS2, RBKS, RGN, RPE, RPIA, |
| Phosphate Signature | TALDO1, TKT, TKTL1, TKTL2 |
| Peroxisome | ABCD2, ABCD3, ACAA1, ACBD5, ACOT8, ACOX1, ACOX2, ACOX3, |
| | ACOXL, AGXT, ATAD1, CAT, CROT, DDO, DECR2, EHHADH, FAR1, |
| | GNPAT, GSTK1, HACL1, HAO1, HAO2, HMGCL, HSD17B4, HSDL2, IDI1, |
| | IDI2, ISOC1, KXD1, LONP2, NUDT12, NUDT19, NUDT7, PAOX, PEX1, |
| | PEX10, PEX11A, PEX11B, PEX11G, PEX12, PEX13, PeX14, PEX16, PEX19, |
| | PEX2, PEX26, PEX3, PEX5, PEX5L, PEX6, PEX7, PHYH, PIPOX, PMVK, |
| | PNPLA8, PXMP2, PXMP4, SCP2, SLC25A17, SZT2, TMEM35A, TTC1, |
| | TYSND1, UGT2A1, ZADH2 |
| ROS Production | GPX1, GPX2, GPX3, GPX5, GPX6 |
| TCA cycle | ACO2, CS, DLAT, DLD, DLST, FH, GLUD1, IDH1, IDH2, IDH3A, IDH3B, |
| | IDH3G, MDH2, MPC1, MPC2, OGDH, OGDHL, PDHA1, PDHA2, PDHB, |
| | PDHX, PDK1, PDK2, PDK3, PDK4, PDP1, PDP2, PDPR, SDHA, SDHAF1, |
| | SDHAF2, SDHAF3, SDHAF4, SDHB, SDHC, SDHD, SUCLA2, SUCLG1, |
| | SUCLG2, SUGCT |
| TNF Signature | ACLY, ACSL1, ADGRE2, AK3, AKAP10, AMPD3, APOL3, ARID3A, |
| | ARSE, ASAP1, B4GALT5, BCL2A1, BHLHE41, BHMT, BIRC3, BRCA1, |
| | CALD1, CASP1, CASP10, CCL15, CCL20, CCL23, CCL3L1, CD37, CD38, |
| | CD83, CDKN3, CKB, CR2, CTNND2, CXCL1, CXCL2, CXCL3, CXCL8, |
| | CYP27B1, DAB2, EBI3, EGR1, EGR2, EPB41, EREG, ETAA1, F3, FABP1, |
| | FBXL2, FCER2, FCGR2A, FLJ11129, FLNA, G0S2, GBP1, GCH1, GJB2, |
| | GLS, GMIP, GP1BA, GRK3, HCAR3, HHEX, HOMER2, HP, ICAM1, IDO1, |
| | IFI44, IKBKG, IL16, IL18, IL1A, IL1B, IL1RN, IL6, INHBA, INSIG1, |
| | ITGA6, KITLG, KLF1, KMO, LGALS3BP, MAP3K4, MARCKS, MGLL, |
| | MMP19, MN1, MRPS15, MSC, MTF1, MX1, NAMPT, NELL2, NFKB1, |
| | NFKB2, NFKBIA, NFKBIZ, NKX3-2, NR3C1, OAS3, PATJ, PDE4DIP, |
| | PDPN, PIAS4, PLAUR, PTGES, PTGS2, RELB, RPGR, RPS9, SDC4, |
| | SERPIND1, SFRP1, SH3BP5, SLAMF1, SLC30A4, SOD2, SPI1, SSPN, STAT4, |
| | TAF15, TAP2, TBX3, TFF1, TNF, TNFAIP2, TNFAIP3, |
| | TNFRSF11A, TRAF1, TSC22D1, TYROBP, UBE2C, VEGFA, WT1 |
| Treg defective | PPP1CA, PPP1CB, PPP1CC |
| TYPE I and | ACSL1, AIM2, APOL3, ATF3, CASP1, CASP10, CCL8, CCND2, CD38, |
| TYPE II IFN | CDKN1A, CFB, CXCL10, CXCL11, EDN1, EPB41, ETV4, F8, GADD45B, |
| Core | GBP1, GBP2, GCH1, GCNT1, GLS, HBG2, IDO1, IFI27, IFI44, IL15, IL15RA, |
| | JAK2, LAP3, LMNB1, CXCL9, MRPS15, MSR1, NKTR, NR3C1, OAS1, |
| | OAS3, PSMB9, PTCH1, RGS1, SERPING1, SOCS1, SP100, STAT1, STAT2, |
| | STX11, TAP1, TAP2, FAS, TNFSF10, UBE2L6, WARS |
| Type I IFN | ACSL1, ADAR, AGT, AIM2, AKAP2, APOBEC3B, APOBEC3G, APOL3, |
| Core Signature | ATF3, ATF5, BAG1, BARD1, BCL7B, BLVRA, BRCA1, BRCA2, BST2, |
| | CAD, CAMK2A, CASP1, CASP10, CASP5, CBR1, CBWD1, CCL13, CCL7, |
| | CCL8, CCNA1, CCND2, CD2AP, CD38, CD4, CD69, CDKN1A, CFB, CHKA, |
| | CNTN6, COL3A1, CTSL, CXCL10, CXCL11, CXCL9, CXCR2, CYP2J2, |
| | DEFB1, DLL1, DSC2, DUSP5, DUSP7, DYNLT1, DYSF, ECE1, EDN1, |
| | EIF2AK2, EIF2B1, EIF4ENIF1, ENPP2, EPB41, ETV4, F8, FAF1, FAS, FGF1, |
| | FLNA, FOXO1, FTL, FUT4, GADD45B, GBAP1, GBP1, GBP2, GCH1, |
| | GCNT1, GLS, GMPR, GPR161, GUK1, HBG2, HIST2H2AA3, HLA-DOA, |
| | HS6ST1, HSP90AA1, IDO1, IFI16, IFI27, IFI35, IFI44, IFI6, IFIT1, IFIT5, |
| | IFITM1, IFITM2, IFITM3, IFRD1, IGL, IKBKG, IL15, IL15RA, IL1RN, IL6, |
| | INPPL1, IRF2, IRF7, ISG15, ISG20, JAK2, JUP, KCNA3, KDELR2, KIF20B, |
| | KLF6, KPNB1, KRT8, LAG3, LAMP3, LAP3, LEPR, LGALS2, LGALS3BP, |
| | LGALS9, LGMN, LMNB1, LMO2, LY6E, MAP2K5, MCL1, MED1, MGLL, |
| | MNDA, MRPS15, MSR1, MX1, MX2, MYD88, NAMPT, NFE2L3, NKTR, |
| | NMI, NR3C1, NUB1, NUPR1, OAS1, OAS2, OAS3, PATJ, PDGFB, PDGFRL, |
| | PKD2, PLSCR1, PMAIP1, PML, PRKRA, PSMB9, PTCH1, RBCK1, RGS1, |
| | RGS6, RTP4, SAT1, SCARB2, SERPING1, SIT1, SOCS1, SP100, SP110, |
| | SP140, SPIB, ST3GAL5, STAP1, STAT1, STAT2, STX11, SUPT3H, TAP1, |
| | TAP2, TARBP1, TCN2, TFDP2, TGM1, TLR3, TLR7, TNFRSF11A, |
| | TNFSF10, TNFSF6, TNK2, TOR1B, TRA2B, TRD, TRIM21, TRIM22, |
| | TRIM34, TRIM38, UBA7, UBE2L6, UBE2S, UNC93B1, USP18, WARS, |
| | WT1, XAF1 |
| Unfolded Protein | B4GALT3, CALR, CALU, CANX, CDS2, CHST12, CHST2, DERL1, DERL2, |
| | DNAJC3, EDEM2, EDEM3, EMC9, ERAP1, ERGIC2, ERO1L, EXT1, |
| | GALNT2, GOLT1B, HERPUD1, HYOU1, IER3IP1, IMPAD1, KDELC1, |
| | KDELR2, LMAN2, LPGAT1, MAN1A1, MANEA, MANF, NUCB2, PDIA4, |
| | PDIA6, PIGK, PPIB, SEC24D, SEC61G, SPCS3, SSR1, SSR3, TRAM1, |
| | TRAM2, UGGT1, XBP1 |
| IFNB1 | ACOD1, CCL7, PROK2, CSTA, IL1B, GPR84, TGM2, IL1RN, IL6, TREM1, |
| ALTERNATIVE PW | MT2A, SAA1, CLEC6A, CXCL2, CSCL3, MARCKSL1, OLR1, MMP14, |
| Increased transcripts | ZNF503, CCL3L3, NR4A1, PHLDB1, SERPINE1, TNF, TREML4, CCL4, |
| | SLC7A5, CLEC4E, FFAR2, PTGES, MEFV, SDC4, EXOC3L4, CD14, |
| | DNMT3L, TNFAIP2, HSPA1B, ARG2, CCL2, BCL3, EPHA4, HCAR2, IL1A, |
| | ACKR3, FMNL2, HSPA1A, IKBKE, MAFF, OSM, RND1, CA13, ICAM1, |
| | ID1, MYBPC2, NR1H3, ARG1, ARHGAP31, SLC39A14, ITGAX, SOD2, |
| | IER3, SLC15A3, FAM20C, Gk, MT1A, TLR2, AEN, CAMKK2, CD86, |
| | COQ10B, DRAM1, ETS2, HBEGF, SLC7A2, TXNRD1, AMBP, CDC42EP2, |
| | HIVEP3, PHLDA1, PIM1, TNFAIP3, TNFSF14, ADORA2B, ASPA, CD207, |
| | CDKN1A, DUSP16, IFI16, ITGA5, PTGIR, RAB20, RAI14, RRS1, |
| | SERPINB2, SOCS3, TRMT61A, URB2, BYSL, CDR2, CTPS1, FCRL5, |
| | MARCO, NOCT, NOP16, RELB, SHMT1, SLC16A10, SNX18, SUSD6, |
| | TFEC, TFRC, TRIM13, B4GALT5, CCRL2, F3, NAB2, NOP2, POGK, |
| | PPRC1, RRP12, SCAMP1, SLAMF8, SLC12A4, SLC25A25, SLC25A33, |
| | SLC2A6, TIMM8A, TMA16, CRYAA, DOT1L, EEF1E1, FPR2, GFPT1, |
| | GRWD1, HEATR1, IRAK3, KPNA2, PDIA6, PVR, SLC20A1, TLNRD1 |
| MS Scoring IL12 | CCL5, CD40LG, CXCL10, CXCL12, CXCR3, GZMB, HAVCR2, HLX, IFNG, |
| | IL12A, IL12B, IL12RB1, IL12RB2, IL2, IL27, IRF4, MAPK14, PHF11, PRF1, |
| | STAT1, STAT4, STOM, TBX21, TYK2, IL2RA, MAP2K3, MAP2K6 |
| MS Scoring IL23 | ABCB1, BATF, CAMK4, CCL20, CCR6, CISH, CREM, CXCL1, IL12B, |
| | IL12RB1, IL17A, IL17F, IL21, IL22, IL23A, IL23R, IL26, IL6, IL6R, IKZF3, |
| | JAK2, KIT, KLRB1, MAF, PRKCA, PTPN13, RORA, RORC, STAT3, TGFB1 |
| MS Scoring IL17 | CCL11, CCL2, CCL20, CCL7, CEBPB, CXCL1, CXCL10, CXCL11, CXCL12, |
| | CXCL2, CXCL5, CXCL6, CXCL8, CXCL9, ICAM1, IL15, IL17A, IL17F, |
| | IL17RA, IL19, IL21, IL6, MAPK1, MAPK12, MAPK14, MAPK3, NFKB1, |
| | NFKBIZ, RELA, SOCS3, TNFSF11, TRAF3, TRAF6, ADAMTS4, CEBPD, |
| | CSF2, CSF3, DEFB4A, EREG, IL17RC, LCN2, MAP3K14, MAPK11, |
| | MAPK13, MMP1, MMP13, MMP3, MMP9, MUC5AC, MUC5B, NOS2, |
| | PTGS2, S100A7, S100A8, S100A9, TIMP1, TRAF3IP2, ABCB1, BATF, |
| | CAMK4, CCR6, CISH, CXCR3, IL12RB1, IL22, IL23A, IL23R, IL26, IL6R, |
| | IKZF3, KIT, KLRB1, MAF, PRKCA, PTPN13, RORA, RORC, STAT3 |

Table 71B provides a list of cell types and sets of genes associated with disease activity in SLE, which were identified using methods and systems of the present disclosure as being strongly correlative with each given cell type. These sets of genes can be used as effective SLE biomarkers to indicate disease activity via the given cell types.

TABLE 71B
Cell Types

| Cell Type | Genes |
|---|---|
| Activated T cells-1 | PRF1, IFNG, EOMES, TBX21, GZMH, CD69, IL2RB, ZNF683, SGK1, TFRC, |
| | TAGAP, GZMB |
| Activated T cells-2 | CD40LG, FASLG, IL17A, IL17F, IL23R, JAKMIP1, KCNA3, KCNN4, |
| | P2RX5, PRKCQ, RELT, RNF125, SATB1, TAGAP, TNFRSF4, TNFRSF9, |
| | CREM |
| Activated T cell-3 | CD69, DPP4, HSPA1B, XCL2, LTA, IKZF3, IL2, JUN, TNFRSF8, TFRC, |
| | ZNF683, IL32, FASLG, PDCD1, IL16, CD40LG, XCL1, IKZF1, CREM, |
| | JAKMIP1, KCNA3, KCNN4, P2RX5, PRKCQ, RELT, RNF125, SATB1, |
| | SLAMF1, TAGAP, TNFRSF4, TNFRSF8, ZC3H12D, HLA-DMA, HLA-DMB, |
| | HLA-DPA1, HLA-DPB1, HLA-DQA1, HLA-DQB1, HLA-DQB2, HLA-DRA, |
| | HLA-DRB1, HLA-DRB3, HLA-DRB4, HLA-DRB5, HLA-DRB6 |
| Activated T cell - 4 | DPP4, FASLG, IL32, LTA, TNFRSF8, ZNF683, CD69, HSPA1B, IL2, JUN, |
| | TFRC, XCL2, IL16, PRDM1, CD40LG, XCL1 |
| Anergic/Activated T cells-1 | ICOS, LAG3, CTLA4, PDCD1, HAVCR2, CD244, CD160, KLRG1 |
| Anergic/Activated T cells-2 | CTLA4, LAG3, TIGIT, CD244, CD160, KLRG1, PBX3, CD96, VSIR |
| Anergic/Activated T cells - 3 | CD160, CD244, KLRG1, PBX3, PDCD1 |
| Anergic/Activated T cells - 4 | CTLA4, LAG3 |
| T circulating | S1PR1 |
| B and Dendritic | TLR10, TLR7, TNFSF4 |
| B Cells-1 | BLK, BLNK, BTLA, CD19, CD22, CD72, CD79A, CD79B, FCRL1, FCRL2, |
| | FCRL5, HLA-DOA, HLA-DOB, MS4A1, PAX5 |
| B cells-2 | IGHD, IGHM, MS4A1, CD79A, GON4L, BANK1, BLK, BLNK, CD22, CD19, |
| | DAPP1, FCRL1, FCRL2, FCRL3, FCRLA, GPR183, KLHL6, PLCL2, |
| | SH3BP5, ZNF318, CD79B, PAX5, VPREB1 |
| CD8T-NK- NKT | CD8B, CRTAM, NKTR, KIR3DL1, KIR3DL2, KLRB1, KLRC3, KLRC4, |
| | KLRD1, KIR2DL3, GNLY, GZMA, GZMB, GZMK, GZMM, HCST, CD2, |
| | CD7, NKG7, RASAL3, TIA1, TXK, CD8A |
| T NK Cell | CD2, GZMK, GZMM, HCST, KIR2DL3, KIR3DL1, KIR3DL2, KLRC3, |
| | KLRC4, KLRF1, CD7, NKTR, RASAL3, TXK |
| Cytotoxic, T cells -1 | PRF1, IFNG, EOMES, TBX21, GZMH, CD69, IL2RB, ZNF683, SGK1, TFRC, |
| | TAGAP, GZMB |
| Cytotoxic T cells-2 | B3GAT1, CRTAM, EOMES, GNLY, GZMA, GZMB, GZMH, IL15RA, TIA1, |
| | TIAL1, TNFSF10, TNFSF12, ZEB2, CTSW, NKG7, PRF1, KLRD1, KLRK1 |
| Dendritic | IGIP, LY75, CLEC10A, CSF1R, LILRA4, CLEC9A, XCR1, CLEC12A |
| Erythrocyte | EPO, GFI1B, GYPA, GYPB, GYPE, ICAM4, NFE2, SLC4A1, TRIM10, |
| | TSPO2, ZNF367 |
| GC B cell | LRMP1, AICDA, DAPP1, RGS13, NUGGC, GCSAM, IRF4, BCL6 |
| Granulocyte | CD177, OSM, RETN, DEFA1, CLC, LTBR4, FUT7, MMP25, CTSS, CXCR2 |
| LDG-1 | BPI, CAMP, CEACAM4, CEACAM6, CEACAM8, CLEC5A, CRISP3, |
| | MS4A3, PGLYRP1, S100A12, S100A8, S100A9 |
| LDG-2 | ARG1, BPI, CAMP, CEACAM8, DEFA4, MPO, OLFM4, S100A12 |
| LDG-3 | AZU1, CEACAM6, CEACAM8, CLEC5A, CTSG, DEFA4, ELANE, LCN2, |
| | LTF, MPO, OLFM4, OSM, RETN, RNASE3, S100A12 |
| LDG-4 | AZU1, CAMP, CEACAM6, CEACAM8, CTSG, DEFA4, ELANE, LCN2, LTF, |
| | MPO, OLFM4, RNASE3 |
| MHC II | HLA-DMA, HLA-DMB, HLA-DPA1, HLA-DPB1, HLA-DPB2, HLA-DQA1, |
| | HLA-DQA2, HLA-DQB1, HLA-DQB2, HLA-DRA, HLA-DRB1, HLA-DRB3, |
| | HLA-DRB4, HLA-DRB5, HLA-DRB6 |
| Monocyte Cell Surface | CD14, CD300C, CD33, CLEC4D, CLEC4E, FCGR1A, FCGR1B, FCGR3B, |
| | CLEC12A, LILRA6, LILRB3, LILRB2, CD68, LILRA5, OSCAR, SEMA4A, |
| | SIGLEC1 |
| Monocytes | BST1, C1QA, C1QB, C1QC, C1R, C1RL, CCL2, CCL8, CD14, CD163, |
| | CD300C, CD33, CLEC4D, CLEC4E, CSF2, CXCL1, CXCL2, CXCR2, |
| | FCGR1A, FCGR1B, FCGR3B, FUT4, GRN, IK, IL18RAP, IL1B, IL1RN, |
| | LILRA5, MNDA, MRC1, OSCAR, S100A8, SEMA4A, SIGLEC1, THBD, |
| | CD68, CLEC12A, LILRA6, LILRB3, LILRB2 |
| Myeloid | TNFAIP8L2, TLR8, S100A8, LTB4R, CFP, S100A9, MPEG1, CD14, |
| | SERPINB8, SIGLEC7, IER3, FCGR3B, PILRA, LILRA6, VSTM1, LILRA2, |
| | MARCH1, CSF2RA, IGSF6, FPR2, CEACAM4, FGL2, CXCL1, CLEC4D, |
| | LILRA1, SIGLEC14, SIGLEC5, CXCL8, CHI3L1, ADGRE3, AOAH, BMX, |
| | CD101, ITGAM, PRAM1, SLAMF8, TNFSF14, TREM1, TREML2 |
| Neutrophil | ARG1, BPI, CAMP, CEACAM4, CEACAM5, CEACAM7, CHIT1, CRISP3, |
| | CXCL5, DEFA1, DEFA1B, DEFA3, DEFB103A, DEFB103B, DEFB106B, |
| | DEFB136, DEFB4A, GRAP2, LBP, MMP8, MS4A3, OLR1, OTOF, |
| | PGLYRP1, PRTN3, S100A8, S100A9 |
| NK | NCAM1, NCR1, NCR3, SH2D1B, KLRF1 |
| pDC-1 | IL3RA, CLEC4C, NRP1 |
| pDC-2 | CLEC4C, NRP1 |
| Plasma Cell-1 | C19orf10, IGH, IGHD, IGHG1, IGHMBP2, IGHV2-5, IGHV4-31, IGH4-34, |
| | IGK, IGKC, IGL, IGLJ3, IGLL1, IGLV@, IGLV1-40, IGLV1-44, IGLV2-14, |
| | IGLV2-5, IGLV3-1, IGLV3-19, IGHV3-20, IGHV3-23, IGLV3-25, IGLV4-3, |
| | IGH4-28, IGLV4-60, IGLV5-45, IGLV6-57, IGLVI-70, MZB1, PRDM1, |
| | THEMIS2, SDC1, IGHA1, IGHA2, IGHE, IGHG3, IGHM, IGHV1-18, IGHV1-2, |
| | IGHV1-46, IGHV3-13, IGHV3-21, IGHV3-33, IGHV3-47, IGHV3-54, |
| | IGHV3-7, IGHV3-72, IGHV3-73, IGHV4-28, IGHV4-30-2, IGHV4-34, |
| | IGHV5-78, IGKV1D-27, IGKV1D-8, IGKV4-1, IGKV5-2, IGLC1, IGLL3P, |
| | IGLL5, IGLV3-10, IGLV7-43, IGLV9-49 |
| Plasma Cell-2 | IGH, IGHA1, IGHA2, IGHD, IGHD3-10, IGHD3-16, IGHE, IGHG1, IGHG2, |
| | IGHG3, IGHG4, IGHGP, IGHJ1, IGHJ2, IGHJ3, IGHJ4, IGHJ5, IGHJ6, |
| | IGHMBP2, IGHV1-18, IGHV1-2, IGHV1-24, IGHV1-45, IGHV1-46, IGHV2-5, |
| | IGHV3-11, IGHV3-15, IGHV3-16, IGHV3-21, IGHV3-38, IGHV3-43, |
| | IGHV3-48, IGHV3-49, IGHV3-53, IGHV3-7, IGHV3-74, IGHV3OR16-8, |
| | IGHV4-28, IGHV4-30-2, IGHV4-34, IGHV4-39, IGHV4-59, IGHV5-51, |
| | IGHV5-78, IGHV7-81, IGKC, IGKJ1, IGKJ2, IGKJ3, IGKJ4, IGKJ5, IGKV@, |
| | IGKV1-16, IGKV1-17, IGKV1-27, IGKV1-5, IGKV1-6, IGKV1-9, IGKV1D-16, |
| | IGKV1D-17, IGKV2D-24, IGKV2D-26, IGKV3-20, IGKV3-7, IGKV3D-20, |
| | IGKV4-1, IGLC2, IGLC6, IGLC7, IGLJ6, IGLL1, IGLL3P, IGLL5, |
| | IGLV1-36, IGLV1-40, IGLV1-47, IGLV1-50, IGLV10-54, IGLV2-11, IGLV2-18, |
| | IGLV2-23, IGLV2-5, IGLV3-1, IGLV3-12, IGLV3-19, IGLV3-21, IGLV3-22 |
| | IGLV3-25, IGLV3-27, IGLV4-60, IGLV4-69, IGLV5-37, IGLV5-45, |
| | IGLV5-48, IGLV6-57, IGLV7-46, IGLV8-61, IGLV9-49, IGLVI-56, IGLVI-70 |
| Plasma Cell-3 | IGLV3-21, IGLV1-40, IGLV2-23, IGHA1, IGLV3-19, IGHV4-30-2, IGHG1, |
| | IGLC2, IGKV1-27, IGHA2, IGKV1-9, IGHV1-24, IGKV1-5, IGKV3-20, |
| | IGLV1-47, IGKV1-6, IGLV3-1, IGKV1-17, IGLV6-57, IGLV2-11, IGHV4-61, |
| | IGLV2-8, IGKJ3, IGKV4-1, IGHV3-33, IGLC7, IGHV4-59, IGHJ2, IGLV3-25, |
| | IGHV3-43, IGHG2, IGLV2-18, IGKJ5, IGHV1-46, IGHV3-13, IGKV1-16, |
| | IGHV3-23, IGHV4-28, IGLV3-27, IGHJ5, IGHV4-39, IGHV5-51, IGHV3-53, |
| | IGHJ3, IGHV3-49, IGHV1-18, IGKJ2, IGKV1D-16, IGKC, IGHG3, IGHV3-15, |
| | IGHJ4, IGHV3-74, IGHV2-5, IGHV6-1, IGHV1-2, IGKJ1, IGHV2-70, |
| | IGHD3-10, IGLV8-61, IGHJ6, IGHV2-26, IGHD3-3, IGHV3-21, IGHV3-72, |
| | IGHV1-45, IGKV1D-8, IGLV10-54, IGHD2-21, IGLV7-43, IGHV3-7, IGKV5-2, |
| | IGHV3-48, IGLV3-16, IGLV3-10, IGHV1-3, IGKV3D-20, IGKV2-24, |
| | IGHJ1, IGLV4-69, IGKJ4, IGHD3-16, IGKV2D-29, IGHD2-15, IGHV3-64, |
| | IGKV2D-26, IGHV3-73, IGHD2-2, IGLV5-37, IGHV4-34, IGKV3D-7, |
| | IGLV1-36, IGHV1-58, IGHV3-20, IGLV9-49, IGLV4-60, IGLV5-45, |
| | IGHV1-69-2, IGKV1D-43, IGLV4-3, IGHG4, IGHV7-81, IGLVI-70, IGLV3-12, |
| | IGHD3-9, IGLJ6, IGKV1D-17, IGKV2D-30, IGLV2-33, IGLV3-32, IGHV3-62 |
| Plasma Cell-4 | IGKV3-20, IGKV1-9, IGLV1-40, IGKJ3, IGHV3-21, IGHV3-49, IGHJ5, |
| | IGHV3-38, IGHV3-53, IGHJ4, IGHV5-51, IGKV4-1, IGKJ2, IGHV3-48, |
| | IGHV3-11, IGHV3OR16-8, IGKV1-6, IGHV4-59, IGLVI-56, IGKJ5, IGLV1-47, |
| | IGHV3-43, IGHJ2, IGLC6, IGHV4-28, IGKV1-17, IGKJ4, IGLV2-11, |
| | IGHV3-23, IGLV2-5, IGHJ3, IGKV1-5, IGLV3-19, IGHJ1, IGKJ1, IGLC7, |
| | IGKV3D-20, IGKV1D-16, IGLC2, IGLV3-1, IGKC, IGHV1-46, IGHV3-74, |
| | IGLV2-23, MIR650, IGKV1-27, IGHV1-18, IGHV2-5, IGHGP, IGLV1-50, |
| | IGLV3-25, IGHV1-45, IGHV4-61, MZB1, IGLV6-57, IRF4, IGLV2-8, IGHV3-33, |
| | IGHV3-15, JCHAIN, IGLV3-21, IGHV3-16, IGHV4-39, IGHG3, |
| | TXNDC11, IGLV3-27, IGHV3-35, IGHG1, ITM2C, IGKV3-7, PDIA4, |
| | IGHV1-24, IGHV4-34, IGLV4-69, IGHV3-7, HSP90B1, IGKV1-16, |
| | HIST1H2BI, IGLV3-9, SEC11C, POU2AF1, IGLV1-36, IGHV3-73, RRM2, |
| | IGHV6-1, IGHG2, HSP90B2P, HIST1H2BM, CD38, DNAJB11, IGHV5-78, |
| | HIST1H2AL, IGLV2-18, HIST1H3G, IGHV2-26, IGHV1-2, HSP90B3P, |
| | IGHV1-58, RRM2P3, UAP1, IGHV4-30-2, TRAM2, HIST1H2AJ, IGLV9-49, |
| | HIST1H1B, DCPS, FKBP11, PDIA6, HIST1H2AG, IGHV2-70, XBP1, |
| | HIST1H3F, HIST1H2AH, PLK1, IGLV7-43, CLPTM1L, MYDGF, IGHA1, |
| | MCM4, KPNA2, HIST1H2BF, HIST1H2AB, HIST1H3J, IGKV2D-24, IGHG4, |
| | IGLV7-46, IGHA2, STMN1, IGHJ6, SLC35B1, SLC1A4, IGKV1D-17, TYMS, |
| | IDH2, IGHD2-15, LOC100421523, IGLV3-12, HIST1H3I, IGHV1-3, PRDX4, |
| | MYBL2, CDC20, HIST1H2BE, FEN1, HIST1H2BH, HIST1H2BN, LRRC59, |
| | IGLV5-45, TPX2, APOBEC3B, IGKV2D-26, NME1, HIST2H2AB, |
| | TIMELESS, MANF, RACGAP1, HIST3H2A, HIST1H2AE, IGLV5-48, |
| | IGHD3-10, MKI67, IGLV8-61, GMPPA, IGLV4-60, IGLV10-54, IGHM, |
| | IGHD3-16, PSAT1, IGHD2-21, MCM2, IGKV3D-7, RRBP1, HIST1H4H, |
| | MCM7, TUBG1P, TUBG1, IGLJ6, TXNDC5, HJURP, CREB3L2, DERL3, |
| | SLC39A7, IGLV5-37, LOC392226, ELL2, ELL2P1, COBLL1, HIST1H4I, |
| | HIST1H2AI, CCNE1, HIST1H3A, IGLL3P, IGLV3-22, IGLVI-70, EZH2, |
| | NUSAP1, WHSC1, SLC37A1, CKS2, H2AFX, TNFRSF13B, TMEM106C, |
| | IGHV7-81, PHGDH, ENTPD7, JUN |
| Plasma Cell-5 | C19orf10, IGH, IGHD, IGHG1, IGHMBP2, IGHV2-5, IGHV4-31, IGH4-34, |
| | IGK, IGKC, IGL, IGLJ3, IGLL1, IGLV@, IGLV1-40, IGLV1-44, IGLV2-14, |
| | IGLV2-5, IGLV3-1, IGLV3-19, IGHV3-20, IGHV3-23, IGLV3-25, IGLV4-3, |
| | IGH4-28, IGLV4-60, IGLV5-45, IGLV6-57, IGLVI-70, MZB1, PRDM1, |
| | THEMIS2, SDC1, TNFRSF17 |
| Plasma Cell-6 | MKI67, IGKC, MZB1, CD38, TYMS, XBP1, JCHAIN, ELL2, CAV1, |
| | TNFRSF17, SDC1 |
| Plasma Cell-7 | SDC1, XBP1, MZB1 |
| Platelets-1 | GP1BA, GP5, GP6, GP9, LY6G6D, MMRN1, PEAR1, PF4, PF4V1, PPBP, |
| | SLC35D3 |
| Platelets-2 | LTBP1, CTTN, CTDSPL, ABLIM3, C6orf25, GUCY1B3, SELP, PTGS1, |
| | ASAP2, TREML1, PDE5A, ALOX12, NRGN, SPARC, ITGA2B, ITGB3, |
| | PRKAR2B, SH3BGRL2, ITGB5, CLU, SDPR, GP6, LY6G6F, TUBB1, |
| | CMTM5, ARHGAP6, GNAZ, TMEM40, DNM3, TTC7B, MFAP3L, PCSK6, |
| | ELOVL7, STON2, PGRMC1, VCL, F13A1, TSPAN33, RHOBTB1, GP1BA, |
| | MYL9, PLA2G12A, TUBA8, MYLK, MGLL, GNG11, RAB27B, MTURN, |
| | MPL, ARHGAP21, GUCY1A3, EHD3, BEND2, ARHGAP18, PF4, LIMS1, |
| | MMD, SIAE, CDC14B, BMP6, TSPAN9, GFI1B, TGFB1I1, DAB2, ESAM, |
| | ANO6, PDLIM1, LINC01151, ABCC3, C1orf198, FHL1, PCYT1B, LGALSL, |
| | PLA2G12AP1, ENDOD1, MAX, LINC00989, PDGFA, C2orf88, SSX2IP, GP9, |
| | PRUNE, ZNF185, SLC6A4, SPX, PARVB, PPBP, INAFM2, CXCR2P1, VIL1, |
| | CABP5, IGF2BP3, SLC24A3, CLEC1B, TPTEP1 |
| T Cells | SH2D1A, TRAC, TRBC1, TRDC, CD247, CCR3, CD226, CD28, CD3D, |
| | CD3E, CD3G, CD5, GATA3, GRAP2, ETS1, LEF1, CD4, CD8B, CD8A |
| T follicular helper | BCL6, CD84, CXCL13, CXCR5, MAF, PDCD1, SH2D1A, ASCL2, BTLA, |
| | ICOS, TNFRSF4 |
| Treg-1 | IKZF2, FOXP3 |
| Treg-2 | ENTPD1, TNFRSF18, FOXP3, IGF2R, IKZF2, IKZF4, ZFP90, ID2, ID3, CLC, |
| | FGL2, LGALS9, TRIM28, USP7 |
| Treg-3 | FOXP3, IKZF2, TNFRSF18, ENTPD1, ID2, ID3, IGF2R, IKZF4, ZFP90, CLC, |
| | LGALS9, FGL2, TRIM28, USP7 |
| TH1 | SPI1, BHLHE40, CCL5, CCR5, CXCL10, CXCL12, CXCR3, EBI3, HLX, |
| | IFNG, IL10, IL12RB2, IL18R1, IL27RA, IRF1, OSM, PHF11, RELA, STAT1, |
| | STAT2, STAT4, STOM, TBX21, TNF, CD300A, HAVCR2 |
| TH17 | ABCB1, CAMK4, CCL20, IL17A, IL17F, IL21, IL21R, IL22, IL23A, IL23R, |
| | IL26, KIT, KLRB1, PRKCA, PTPN13, RORA, RORC, RUNX1 |
| TH17 - 2 | IL21R, ABCB1, CAMK4, CCL20, IL12RB1, IL17A, IL17F, IL21, IL22, |
| | IL23A, IL23R, IL26, IL6R, KLRB1, PTPN13, RORA, RORC, RUNX1, STAT3 |
| TH2 | BATF3, PTGDR2, CXCR4, GATA3, IL13, IL4, IL4R, IL5, NFIL3, SGK1, |
| | STAT6, WHSC1 |
| Circulating Tfh | CXCL13, CXCR5, SH2D1A, ASCL2, BCL6, CD84, MAF, PDCD1, ICOS, |
| | BTLA, TNFRSF4 |
| TH1 short | CXCR3, TBX21, IFNG, CXCR5 |
| TH2 short | GATA3, IL4, IL5, IL13 |
| TH17 short | CCR6, RORGT, IL17A, IL22 |
| TH1/TH17 short | CXCR3, CCCR6, IFNG, IL17A, IL22 |
| T-resting | CCR7, CCR1, SELL |
| TSLE | TNFSF13B, LGALS3BP |
| Tself-renewal | LEF1, MYC |
| Treg defective | PPP1CC, PPP1CA, PPP1CB |
| Treg and TH2 | STAT5a, STAT5b |
| CD4 Module Neg | POLR1E, HADHA, BAG3, RGCC, AK5, CCR2, CD44, FBL, KCTD7, CUX1, |
| | DPEP2, SSBP2, ERGIC3, GPR183, SESN1, FHL1, BACH2, PDCD4-AS1, |
| | NOSIP, TOMM7, PTDSS1, SH3YL1, SSR2, KLHL22 |
| CD4 Module Pos | NKG7, TBX21, GZMH, PRF1, CCL4, CST7, CX3CR1, FGR, ADRB2, |
| | TGFBR3, TPST2, ADGRG1, GZMA, PRSS23, GZMB, SRGN, CCL5, PLOD1, |
| | PLEK, GNLY, APOBEC3G, SPON2, KLRD1, PRNP, TMX4 |
| IG CHAINS | IGLV3-21, IGLV1-40, IGLV2-23, IGHA1, IGLV3-19, IGHV4-30-2, IGHG1, |
| | IGLC2, IGKV1-27, IGHA2, IGKV1-9, IGHV1-24, IGKV1-5, IGKV3-20, |
| | IGLV1-47, IGKV1-6, IGLV3-1, IGKV1-17, IGLV6-57, IGLV2-11, IGHV4-61, |
| | IGLV2-8, IGKJ3, IGKV4-1, IGHV3-33, IGLC7, IGHV4-59, IGHJ2, IGLV3-25, |
| | IGHV3-43, IGHG2, IGLV2-18, IGKJ5, IGHV1-46, IGHV3-13, IGKV1-16, |
| | IGHV3-23, IGHV4-28, IGLV3-27, IGHJ5, IGHV4-39, IGHV5-51, IGHV3-53, |
| | IGHJ3, IGHV3-49, IGHV1-18, IGKJ2, IGKV1D-16, IGKC, IGHG3, IGHV3-15, |
| | IGHJ4, IGHV3-74, IGHV2-5, IGHV6-1, IGHV1-2, IGKJ1, IGHV2-70, |
| | IGHD3-10, IGLV8-61, IGHJ6, IGHV2-26, IGHD3-3, IGHV3-21, IGHV3-72, |
| | IGHV1-45, IGKV1D-8, IGLV10-54, IGHD2-21, IGLV7-43, IGHV3-7, IGKV5-2, |
| | IGHV3-48, IGLV3-16, IGLV3-10, IGHV1-3, IGKV3D-20, IGKV2-24, |
| | IGHJ1, IGLV4-69, IGKJ4, IGHD3-16, IGKV2D-29, IGHD2-15, IGHV3-64, |
| | IGKV2D-26, IGHV3-73, IGHD2-2, IGLV5-37, IGHV4-34, IGKV3D-7, |
| | IGLV1-36, IGHV1-58, IGHV3-20, IGLV9-49, IGLV4-60, IGLV5-45, IGHV1-69-2, |
| | IGKV1D-43, IGLV4-3, IGHG4, IGHV7-81, IGLVI-70, IGLV3-12, |
| | IGHD3-9, IGLJ6, IGKV1D-17, IGKV2D-30, IGLV2-33, IGLV3-32, IGHV3-62 |
| TCRG | TRGC2, TRGV1, TRGV2, TRGV3, TRGV4, TRGV5, TRGV7, TRGV8, |
| | TRGV9, TRGV10, TRGV11 |
| TCRD | TRDV3, TRDV2, TRDV1, TRDJ4, TRDJ3, TRDJ2, TRDJ1, TRDC |
| TCRA | TRAV41, TRAV40, TRAV39, TRAV38-2DV8, TRAV38-1, TRAV36DV7, |
| | TRAV35, TRAV34, TRAV30, TRAV29DV5, TRAV27, TRAV26-2, TRAV26-1, |
| | TRAV25, TRAV24, TRAV23DV6, TRAV22, TRAV21, TRAV20, TRAV19, |
| | TRAV18, TRAV17, TRAV16, TRAV14DV4, TRAV13-2, TRAV13-1, |
| | TRAV12-3, TRAV12-2, TRAV12-1, TRAV10, TRAV9-2, TRAV9-1, TRAV8-7, |
| | TRAV8-6, TRAV8-4, TRAV8-3, TRAV8-2, TRAV8-1, TRAV7, TRAV5, |
| | TRAV4, TRAV3, TRAV2, TRAV1-2, TRAV1-1 |
| TCRB | TRBV28, TRBV27, TRBV25-1, TRBV24-1, TRBV23-1, TRBV21-1, TRBV20-1, |
| | TRBV19, TRBV11-2, TRBV11-1, TRBV10-2, TRBV10-1, TRBV9, TRBV7-7, |
| | TRBV7-6, TRBV7-5, TRBV7-4, TRBV7-3, TRBV7-1, TRBV6-8, TRBV6-7, |
| | TRBV6-6, TRBV6-5, TRBV6-4, TRBV6-1, TRBV5-7, TRBV5-6, TRBV5-5, |
| | TRBV5-4, TRBV5-3, TRBV5-1, TRBV4-2, TRBV4-1, TRBV3-1, TRBV2, |
| | TRBV1, TRBJ2-7, TRBJ2-6, TRBJ2-5, TRBJ2-4, TRBJ2-3, TRBJ2-2P, TRBJ2-2, |
| | TRBJ2-1, TRBC2 |
| TCRAJ | TRAJ61, TRAJ59, TRAJ58, TRAJ57, TRAJ56, TRAJ54, TRAJ53, TRAJ52, |
| | TRAJ50, TRAJ49, TRAJ48, TRAJ47, TRAJ46, TRAJ45, TRAJ44, TRAJ43, |
| | TRAJ42, TRAJ41, TRAJ40, TRAJ39, TRAJ38, TRAJ37, TRAJ36, TRAJ35, |
| | TRAJ34, TRAJ33, TRAJ32, TRAJ31, TRAJ30, TRAJ29, TRAJ28, TRAJ27, |
| | TRAJ26, TRAJ25, TRAJ24, TRAJ23, TRAJ22, TRAJ21, TRAJ20, TRAJ19, |
| | TRAJ18, TRAJ17, TRAJ16, TRAJ15, TRAJ14, TRAJ13, TRAJ12, TRAJ11, |
| | TRAJ10, TRAJ9, TRAJ8, TRAJ7, TRAJ6, TRAJ5, TRAJ4, TRAJ3 |

Table 71C provides a list of mouse cell types and sets of genes associated with disease activity in SLE, which were identified using methods and systems of the present disclosure as being strongly correlative with each given mouse cell type (e.g., using a mouse disease model for SLE). These sets of genes can be used as effective SLE biomarkers to indicate disease activity via the given mouse cell types.

TABLE 71C
Mouse

| Cell Type | Genes |
|---|---|
| B cells | Tnfrsf8, Slamf1, Havcr1, Tlr7, Irf4, Tlr9, Sh2b2, Samsn1, Pou2af1, Btk, |
| | Klhl6, Pkn1, Blk, Blnk, Tnfsf7, Tnfrs13c, Cd22, Elf1, Snap23, Ncf1, H2- |
| | Ob, Ms4a1 |
| Monocytes/Macrophages | Tlr8, Tnfaip3, Tnip3, Ace, Ms4a4a, Clec4e, Cd300e, Clec4a3, Tnfrsf1b, |
| | Vsig4, Lilra5, Ms4a2, Tgm2, Msr1, Hmmr, Pilra, Igsf6, Siglec1, Clec4d, |
| | Fpr2, Csf1r, Clec7a, Adgre1, Smpdl3b, Adamdec1, Cd51, Cfb, Il18bp, Il10, |
| | Il12b, Ctla2b, Il27, C6, Serping1, Cybb, Cxcl10, Slc11a1, Lmnb1, Hvcn1, |
| | Mpeg1, Clec4n, Csf2rb2, Lyz1, Fcgr1, Fcgr3 |
| Dendritic | Cd300e, Vsig4, Hmmr, Igsf6, Adamdec1, Il18bp, Il12b, Il27, Themis2, |
| | Cd180, Slamf1, Il21r, Cd83, Cnr2, Ulbp1, Fcgr1 |
| Myeloid | Ms4a4a, Clec7a, Pik3ap1, Btk, Fgr, Bach1, Treml4, Ms4a6c, Itgax, Apoc1, |
| | Slpi, Gm15931, Lilrb4a, Cd300lb, Cd300ld |
| Plasma Cells | Jchain, Hmmr, Hvcn1, Cd38, Slamf7, Hyou1, Fkbp11, Mzb1, Ighg1, Igkc, |
| | Tnfrsf17, Ighd, Stil, Parpbp, Ighd1-1, Ighd2-7, Ighe, Ighg3, Ighj1, Ighj2, |
| | Ighj3, Ighv1-12, Ighv1-14, Ighv1-18, Ighv1-19, Ighv1-2, Ighv1-20, Ighv1-21, |
| | Ighv1-21-1, Ighv1-22, Ighv1-25, Ighv1-26, Ighv1-28, Ighv1-30, Ighv1-31, |
| | Ighv1-33, Ighv1-36, Ighv1-37, Ighv1-39, Ighv1-4, Ighv1-42, Ighv1-43, |
| | Ighv1-47, Ighv1-5, Ighv8-8, Igip, Igkj2, Igkj3 |
| PRR Signaling | Tlr13, Oasl2, Oas2, Tlr8, Tlr13, Oas3, Tlr7, Zbp1, Dusp16, Irf7, Oasl2, |
| | Tirap, Isg15, Irf8, Nlrc3, Irf4, Nlrc5, Tlr9, Irak4, Trim14, Lrrfip1, Ticam2, |
| | Irf9, Ddx58, Nlrp3, Tnfaip3, Tlr4, Casp4, Ifih1, Nod2, Zc3hav1, Traf3, |
| | Arl16, Rnf41, Irak3, Myd88, Trim35, Tank, Ifi213 |
| IFN gene Signature (IGS) | Mx1/Mx2, Ifi44, Rsad2, Rtp4, Eif2ak2, Ifitm3, Sp110, Gbp2, Sp100, |
| | Cmpk2, Ifit1, Ifit3b |
| Pro Cell Cycle | Cep55, Ncapg, Nek2, Cep85l, Cdca2, Prc1, Ndc80, Foxm1, Ect2, Clspn, |
| | Ttk, Esco2, Bub1, Dsn1, Top1, Fen1, Kntc1, Ncaph, Aurkb, Ska3, Cdc20, |
| | Cenpe, Kif11, Cdca5, Top2a, Cdc42bpb, Bub1b, Uhrf1, Incenp, Spdl1, |
| | E2f2, Cdc45, Nusap1, Dbf4, Sgo1, Mis18bp1, Espl1, Cenpl, Ccnd2, Cep57, |
| | Lats2, E2f3, Mcm6, Cep68, Cdk14, Rfc1, Ccnd1, Cdc25b, Mcm5, Mis12, |
| | Helb, Mcmbp, Cenpj, Cdc27, Plk3, Nek7, Pold1, Nde1, Mcm4, Ccng1, |
| | Ccnb1-ps, Cenpc1, C330027C09Rik |
| Unfolded Protein & Stress | Chac1, Bhlha15, Xbp1, Edem1, Creb312, Derl3, Sel1l, Hspa5, Edem3, |
| | Dnajc3, Insig1, Ube2j1, Edem2, Dnajb9, Erp44, Hsph1, Rpn1, Herpud1, |
| | Man1b1, Vmp1, Ubxn4, Pdia4, Tmem214, Calr, Atf6, Erlec1, Canx, Sec63, |
| | Vcp, St13, Nploc4, Ero1l, Ero1lb |
| Endosome and Vesicles | Dnm3, Zfyve9, Itsn2, Ehd4, Dab2, Smap1, Capza1, Washc4, Eea1, Arfgap3, |
| | Snx2, Arpc1b, Arap1, Cyth4, Rab5a, Vps26a, Arfgap2, Git1, Rab35, |
| | Vps26b |
| Endoplasmic Reticulum | Edem1, Edem3, Edem2, Erp44, Erlec1, Creld2, Ddn, Ryr3, Hsp90b1, |
| | Sec24d, Kcnrg, Tram2, Txndc11, Txndc5, Sdf2l1, Hyou1, Sec24a, Prr11, |
| | Hspa13, Dnajb11, Manf, Sec61a1, Pdia6, Atp2a2, Lrrc59, Mtdh, Tor3a, |
| | Ssr4, Pdia3, Slc35e1, Slc35b1, Atp10d, Plod2, Ergic1, Sec23a, Surf4, |
| | Sec11c, Mlec, Rrbp1, Erap1, Lclat1, Slc33a1, Ssr3, Ttc9c, Ergic2, Stt3a, |
| | Sqle, Alg2, Elovl5, Clptm1l, Ssr1, Gdap2, Sec23b, Spcs3, Trim59, Mia2, |
| | Srp72, Soat1, Yipf5, Kctd20, Dnajc14, Sec23ip, Stim2, Aldh3a2, Ankrd13c, |
| | Ero1l, Ero1lb, Deaf1, Cyp51, Rab1 |
| Golgi | Fam20c, Mest, Slc9a7, Atp7a, Cgnl1, B3gnt9, Plagl1, Chst2, Fgd4, B3galt1, |
| | Chst3, Glcci1, Ica1l, Fndc3b, Xylt1, Chst1, Fut8, Man2a1, Slc39a7, Rab43, |
| | Manea, Atp8b2, Gcnt2, Parp9, Cyb5d1, Rab39b, Bhlhe40, B4galt5, Qpctl, |
| | Itpripl2, Uso1, Mgat2, Cog5, Serinc5, Tpst1, Sec14l1, Slc30a7, Tmed5, |
| | Pask, Gcnt1, Pdxdc1, Psen1, Rnf157, G2e3, Ddhd1, B3gnt5, Man1a2, |
| | Syngr2, St6galnac4, Gsap, Arcn1, St8sia4, Tmf1, Bicd2, Gga2, Pde4dip, |
| | Slc38a10, Cnst, Alkbh5, Copb2, Rab30, C1galt1, Fam20b, Chpf2, Tm9sf3, |
| | Slc30a5, Gorasp2, Fut11, Osbpl9, Atp2c1, Copb1, Vps54, Gosr2, Copa, |
| | Stip1, Copg1, Tmed10, Arl1, Calu, Pcsk7, Gdi2, Furin, Gpr107, Gga1, |
| | Man1a |
| Integrin Signaling | Gna13, Lamc1, Itgav, Plcg2, Spock2, Raf1, Col17a1, Actg2, Lamc3, |
| | Bcar1, Itgb8, Lama1, Col5a2, Hspg2, Fbn2, Eln, Col4a1, Itgae, Vcan, |
| | Col4a2, Col3a1, Col13a1, Spon1, Ecm1 |
| Cytoskeleton | Sgce, Mapt, Krt18, Lrch2, Nphp1, Pfn4, Spata7, Dync2li1, Kif17, Spire2, |
| | Ang, Snph, Krt86, Mapre3, Dlg4, Homer2, Eda, Myo1d, Actn2, Plekhg4, |
| | Ptpdc1, Kif7, Tnni1, Ank3, Tubb3, Ift81, Gpr4, Dock6, Ttc8, Ttc12, Wdr35, |
| | Epb41l4b, Ehbp1, Spag4, Odf2l, Mylpf, Ift43, Nefh, Wdr60, Ttll1, Dennd2a, |
| | Wdr19, Vill, Kptn, Pls3, Nek3, Bbs2, Kifc2, Bbs1, Dmd, Arl6, |
| | Dzip1, Fuz, Fnbp1l, Rpgr, Bbs9, Tubb4a, Kif9, Ccdc14, Palld, Bbs4, Nek8, |
| | Krt10, Arhgap18, Ift74, Hook2, Dctn6, Tubg2, Eml2, Ccdc114, Gsn, Cnn3, |
| | Matk, Katnal1, Cep72, Mks1, Klc4, Pick1, Kifap3, Cep57l1, Sgcb, Klhdc1, |
| | Ift122, Lrrc45, Vmac, Fntb, Sfi1, Cep41, Tube1, Spata6, Cep19, Mob3b, |
| | Cep131, Fhl3, Tuba4a, Ccdc61, Ick, Ift27, Ip6k2, Marveld1, Ankra2, |
| | Pdlim1, Tpm1, Tubb4a |
| Transporters | Slc6a1, Slc51a, Kcnk12, Slc27a6, Slc12a5, Atp4a, Slc6a13, Kcnab3, |
| | Slc22a18, Kcnh3, Kcnh2, Slc22a17, Slc7a4, Aqp9, Abcg2, Fxyd1, Slc44a5, |
| | Kcnd1, Cacna1c, Tfr2, Cacng8, Aqp11, Aqp1, Trpm1, Kcng2, Slc2a10, |
| | Abcb4, Kcnh7, Clcn1, Slc27a1, Clcn2, Slc38a5, Kcne3, Slc39a8, Kcnip3, |
| | Akap7, Kcnc3, Kctd14, Slc41a3, Aqp3, Tesc, Slc16a7, Slc14a1, Slc9a5, |
| | Kcnj8, Cacna1a, Cnga1, Spns3, Slc29a2, Slc43a1, Slc4a8, Slc16a5, Slc29a4, |
| | Cbarp, Bspry, Slco2b1, Stom, Cacna2d2, Cacnb1, Slc29a1, Ano10, Slc39a4, |
| | Slc2a9, Slco3a1, Kctd12, Atp9a, Slc50a1, Slc19a1, Kctd2, Kctd13, Slc39a3, |
| | Ttyh3, Slc6a20a, Ank, Cbarp, Gm44509 |
| Endosome and Vesicles | Syt3, Syngr1, Rab3b, Snx31, Clstn1, Syt5, Sytl1, Unc13b, Pacsin3, Spag8, |
| | Scg5, Prss16, Fam109b, Vamp5, Rab17, Rab38, Ocrl, Slc9a9, Snx22, |
| | Dennd6b, Arl4c, Tmem9, Tmem163, Sytl3, Stx2, Appl2, Rab27b, Stxbp1, |
| | Abca5, Mamdc4, Rab23, Als2cl, Scrn2, Sft2d3, Ap4b1, Lamtor2, Rab24, |
| | Hap1, Flot2, Dennd1a, Rab3d, Fcho1, Rabep2, Vps16, Ap1b1, Ap1g2 |
| Mitochondria | Gpat2, Gls2, Amt, Cyp11a1, Nme4, Dhtkd1, Me3, Nmnat3, Maoa, Clybl, |
| | Tmlhe, Slc25a23, Tdrkh, Ldhd, Hmgcs2, Fahd1, Bphl, Chchd6, Aldh5a1, |
| | Mthfd2l, Acad10, Pyroxd2, Slc25a35, Hint2, Bckdhb, Lipt1, Coq4, |
| | Nipsnap1, Cyp27a1, Mccc2, Aldh4a1, Pccb, Iba57, Ppox, Glrx5, Amacr, |
| | Ethe1, Acp6, Lyrm1, Sfxn2, Dguok, Agk, Sfxn4, Mcee, Immp2l, Clpb, Ivd, |
| | Mtfr1l, Naxe, Adck5, Sfxn5, Pcca, Coq7, Ppa2, Akap1, Mccc1, Acadsb, |
| | Ccdc58, Slc25a24, Fxn, Suox, Acad11, Slc25a39, Pstk, Acad8, Fpgs, Coq2, |
| | Timm10b, Tk2, Taz, Mipep, Dhodh, Adck1, Abcb8, 2310061I04Rik, |
| | 1700021F05Rik |
| Fatty Acid Biosynthesis | Echdc2, Acss2, Hadh, Mecr, Decr1, Pcx |
| Peroxisomes | Gstk1, Nudt12, Hacl1, Pex11a, Paox, Pex6, Pex11b, Pex7, Pxmp4, Pex5 |
| ROS Protection | Prdx2, Txnrd2, Gstp1, Prdx6 |
| GC markers | Gcsam, Nuggc, Rgs13, Klhl6, Aicda, Bcl6, irf4 |
| T follicular helper | Bcl6, Pdcd1, Ascl2, Icos, Tnfsf4 |

Table 71D provides a list of patient ancestry and sex categories, and sets of genes associated with disease activity in SLE, which were identified using methods and systems of the present disclosure as being strongly correlative with each given patient ancestry or sex category. These sets of genes can be used as effective SLE biomarkers among patients within each given patient ancestry or sex category.

TABLE 71D
Patient Ancestry/Sex

| | |
|---|---|
| Sex Female | XIST, TSIX, JPX |
| Sex Male | PRKY, TTTY14, CD24P4, UTY, USP9Y |
| European Ancestry | CES1, TMEM187, APLP2, RHOG, |
| | MID1IP1, FCN1, ALDH2, LAPTM5, |
| | CAP1, ZBED1 |
| Not African Ancestry | ACKR1, CD36, G6PD |
| African Ancestry | P4FV1, TUBB2A, MICALCL, OSBP2, |
| | NFIX, FAM46C |
| Native American Ancestry | NDUFB3, TNFAIP6, ANXA3, VPS29, |
| | C1GALT1, NOD2, FCGR1B |

Table 71E provides a list of primary immunodeficiency (PID)-associated clusters, and sets of genes associated with disease activity in SLE, which were identified using methods and systems of the present disclosure as being strongly correlative with each given PID-associated cluster. These sets of genes can be used as effective SLE biomarkers to indicate disease activity via the given PID clusters.

TABLE 71E
PID Gene Clusters

| PID Cluster | Genes |
|---|---|
| Immune cell surface and | IFNG, IL7R, IL6, ADAM17, MPO, IL17A, IL17F, FAS, |
| secreted immune | CD40LG, CD70, CXCR4, TLR3, IL17RC, IL18, IL10, CD40, |
| | CD27, STAT5B, CD19, CD79A, SLC11A1, TNFSF11, IL12B, |
| | TNFSF10, IL2RA, CCL2, TNFRSF1A, CR2, ITGAX, |
| | FCGR3B, FCGR3A, FASLG, PIK3CD, ITGAM, MYD88, |
| | PTPRC, FOXP3, TNFRSF4, STAT3, IL10RA, STAT1, CCL22, |
| | CTLA4, C3, IRF8 |
| Immune signaling | ZAP70, CD8A, RAG1, RAG2, CD3E, BCL11B, NOD2, LCK, |
| | ACP5, NFKB1, ITK, UNC119, TFRC, IRF7 |
| Pattern recognition | MVK, IRF3, IRAK4, CASP8, TNFAIP3, NFKBIA, NLRP1, |
| receptors and | NLRP3, IRAK1, CSF2RA, RELB, XIAP, IL17RA, TYK2, |
| intracellular signaling | CTSC, CD3G, FADD, IL1RN, PTPN6, FCGR1A, IL2RG, |
| | UNC93B1, TRAF3, TIRAP, MAP3K14, NFKB2, CLEC7A, |
| | TICAM1, IFNAR2, IL10RB, IL12RB1, IFNGR1, JAK1, JAK3, |
| | IKBKG, CASP10, IKBKB, IFIH1, MEFV, AIRE |
| DNA repair | KRAS, POLE2, POLE1, STN1, CBL, DNASE2, ELF4, |
| | TGFBR2, CHD7, TCF3, CSF2RB, CYBA, LIG4, LIG1, CD81, |
| | DCLRE1C, MRE11A, ERBIN, GINS1, FAAP24, PCNA, |
| | VAV1, CD3D, ATM, POLA1, XRCC4, RTEL1, DNA2, |
| | NEIL3, RUNX1, NSMCE3, B2M, PNP, PRF1, TRAF3IP2, |
| | SMARCAL1, CD247, RAD52, SH3BP2, NFAT5, MCM4, |
| | HAX1, DNMT3B, KMT2A, RNASEH2A, BLM, MSH6, NBN, |
| | CSF3R, TAOK2, PMS2, NRAS, UNG, RNF168, TMEM173, |
| | HELLS, ADA2, NHEJ1, HMOX1, GATA2, CDCA7, PRKDC, |
| | TBK1 |
| Cluster5 | CFHR4, THBD, PIGA |
| Secreted immune | COLEC11, RASGRP2, TRAC, FCN3, C1QC, CFHR1, CD55, |
| | C1QA, C1QB, CD46, C8G, C8A, C8B, TNFRSF13B, |
| | SH2D1A, C4A, C1R, C1S, ITGB2, F12, ICOS, C9, C7, C6, C2, |
| | CFI, CFH, SERPING1, C4BPB |
| Immune signaling | USP18, SAMHD1, MS4A1, MYH9, IRF4, TGFBR1, MKL1, |
| | BLNK, ADAR, ISG15, DNAJC21, RASGRP1, STK4, CIB1, |
| | WAS, TNFRSF13C, IL21, MYO5A, TREX1, TNFSF12, |
| | CD79B, PIK3R1, CFTR, BTK, ACTB, IKZF1, GFI1, |
| | LRRC8A, IL21R, NLRC4, CEBPB, SMARCD2, MBL2, |
| | PGM3, PSTPIP1, PTEN, CIITA, LAT, AICDA, TNFRSF11A, |
| | RNASEH2B, RNASEH2C, IL6ST, NCF4, IGLL1, TARBP2, PSMB8, |
| | STAT2, IL12RB2, APOL1, RNASEL, IFNGR2, |
| | ARPC1B, RET, ADA, INO80, LRBA, HEXIM1, ELANE, |
| | PLCG2, TERT, PRKCD, MYB, VPREB1, BACH2, C4BPA, |
| | WIPF1 |
| Endosome and vesicles | MLPH, STX11, AP3B1, RAB27A, STXBP2, SLC29A3, LYST, |
| | UNC13D |
| Lysosome | CLCN7, ATP6AP1, ATP6V0A2, VPS45, TCIRG1, PLEKHM1, |
| | OSTM1 |
| Secreted and ECM | MASP1, MASP2, CFHR5, CFHR3, CFHR2, CD59, C4B, C5, |
| | CFP, CFD, CFB |
| DNA Repair | FANCE, FANCC, FANCA |
| Nucleus and nucleolus | DKC1, DCLRE1B, TINF2, WRAP53, NOP10, TPP1, NHP2, |
| | ACD, CTC1 |
| Endoplasmic reticulum | DNAI1, GIF, JAGN1, TTC37, SEC61A1, TAZ, SKTV2L, |
| | SBDS, TTC7A, NLRP12, MAGT1, TECR, MOGS, RPL35A, |
| | RPSA, RORC, EXTL3, SRP54, HYOU1, RPL5 |
| Ubiquitylation-and- | CARD11, CARD14, OTULIN, ITCH, MALT1, RNF31, |
| Sumoylation and | CARD9, BCL10, RBCK1 |
| intracellular signaling | |
| Cytoskeleton | DOCK2, DOCK8, MSN, COPA, CYBB, RHOH, RAC1, |
| | RAC2, PLXNA1, CORO1A, ROR2, NCF2, NCF1, FERMT3, |
| | FPR1 |
| Interferon stimulated genes | MX2, MYSM1, SAMD9L, SP110, TRIM25, SAMD9 |
| Glycolysis- | G6PC, G6PD, PEPD, WDR1, G6PC3, GPI, CLPB |
| Gluconeogenesis-and- | |
| Pentose-Phosphate- | |
| Pathways | |
| MHC Class 1 | TAPBP, RFXAP, RFXANK, TAP1, TAP2, RFX5 |
| Golgi | PSEN1, PSENEN, NCSTN |
| Cytoplasm-and-Biochemistry | CTPS1, RANBP2, DSP, AK2, TCN2, MTHFD1 |

Table 71F provides a list of plasma cell (PC)-associated clusters, and sets of genes associated with disease activity in SLE, which were identified using methods and systems of the present disclosure as being strongly correlative with each given PC-associated cluster. These sets of genes can be used as effective SLE biomarkers to indicate disease activity via the given PC clusters.

TABLE 71F
Lugar PC Derived

| PC-Derived Cluster | Genes |
|---|---|
| SLE Unique | ASPM, CDC20, BIK, SLC7A11, PIK3CG, RGS13, BUB1, HMGN5, MKI67, |
| Filtered UP | GGH, BMP8B, NCAPG, 10-Sep, HIST1H2BB, ADA, SCUBE3, FAM149A, |
| | NEUROG3, KIF20A, CLIC3, COL9A3, HIST1H4B, CD320, CDC25A, NUSAP1, |
| | HIST1H4L, CD27, NANS, CCNA2, IL6R, CEP55, PTTG3P, DLGAP5, SEMA4A, |
| | KCNK12, CENPN, IDH2, TIMM44, TRAT1, HJURP, CA6, NEK2, COX11, |
| | FZD7, E2F8, HMMR, LGSN, TLX3, PTPRD, KDELC1, PDE1A, MELK, CCR10, |
| | TK1, SERPINF1, PERP, GRIK1, IL1R1, SRMP1, UCHL1, RGS16, MUC5B, |
| | MCUR1, CDCA3, EFS, IGLL1, KLF10, MCM10, SLC27A2, CTNNAL1, |
| | FBXO5, CDC42BPA, GPRC5D, KIFC1, UCK2, GC, BUB1B, PTTG1, RS1, |
| | GPR15, PSAT1, BAZ1B, CYP11B2, KLK11, ERCC6L, CEP97, TUBG1, BIRC5, |
| | SLC35B1, ATP1A2, PLPBP, IQSEC2, AMOTL2, CDKN3, AURKA, SPRR1A, |
| | SMAD7, HRH1, UGGT2, GNAS, FOLH1B, GSPT1, ATF5, FAXDC2, NCOA3, |
| | AAK1, PKP4, FA2H, RAB27A, CDKN2C, DCPS, MCM3AP, SLCO4A1, |
| | UQCRQ, HSD11B2, DTL, CCNB1, PHGDH, PKD1P6, SAR1A, TRIP13, |
| | MTNR1A, CSF2RB, SLC19A1, CDC6, HPX, FEN1, CHEK1, LRRC59, PPA1, |
| | MTRR, CCNE2, ZNF593, CCNC, MIF, TPP2, UBE2C, SEC13, TRIP6, AGK, |
| | COX7A2, FUT8, TPST2, GARS, FDX1, NDUFB6, GLRX5 |
| SLE Unique | FAM208A, OPN3, LIPA, LAPTM5, BANK1, PAX5, PKIG, SNX2, DENND5A, |
| Filtered UP | ZNF83, CD19, SNN, CNPPD1, LYL1, ABLIM1, MS4A1, NAIP, SNAP23, |
| | PIKFYVE, FOXO4, RNF41, ANKZF1, SIDT2, CLIP2, HLA-DOB, FAM20B, |
| | SYNPO, IRF5, KDM4B, KIF21B, BIN1, FCMR, SMG1, SETMAR, CACNA1A, |
| | LBH, DCUN1D4, PHC1, GPD1L, NOTCH2, LINC00472, ZBTB18, RNF141, |
| | FCGR2C, AKT3, NCR3, TMEM127, NT5E, NOTCH2NL, SH3BP2, CLCN4, |
| | RIN3, UNKL, MNDA, ZEB2, HCK, CEP170, ZNF236, ZNF318, ZSCAN18, |
| | PCDH9, TNFSF12, CBR3, HS3ST1, XIST, SUSD5, CD72 |
| Overlap | PTK2, CD22, ARPC4, ELF4, PDE4DIP, CERS4, BCL11A, ARHGAP17, |
| Filtered Down | PLEKHA1, SIPA1L1, CCR6, HHEX, IRF8, BLK, MFHAS1, SPIB, ABR, STX7, |
| | CD37, PIK3CD, DEK, CCND3 |
| Overlap | R3HCC1, PREB, EDEM2, TECR, IGKV1D-13, NEU1, CNPY2, SPCS1, ESPL1, |
| Filtered UP | NUS1P3, COPB2, ALG9, NES, LMAN2, GUSBP11, ST6GALNAC4, SPCS2, |
| | SEC23B, IFNAR2, GMPPA, SPATS2, TMBIM6, HSD17B8, MGAT2, STT3A, |
| | LAX1, CHST2, ALG5, IGLV2-14, SEC61B, PDIA6, TM9SF1, TRAM1, CD6, |
| | BCAN, SEC61G, KIR2DL4, PYCR1, ESR1, SLC1A7, CYP2E1, ADGRB1, |
| | HIST1H2BG, TMEM208, B4GALT3, RPN1, TP63, RWDD2A, IQGAP2, IGKV1-17, |
| | ERV9-1, XCL1, C11orf80, ANKS1B, ARF4, DRD4, SLCO2B1, ATP11A, |
| | POU6F2, EDEM3, GMPPB, DERL1, MBNL2, LMAN1, KCNJ5, SSR4, YIPF2, |
| | TRAM2, STARD5, DERL2, B9D1, 4-Sep, CYP26A1, ST3GAL6, RBM47, |
| | RHBDD3, APOA4, GAB1, TXNDC15, NAT2, CADM3, CHPF, SSR1, TIMP4, |
| | SSR3, DOK4, LZTS1, IGKV1D-8, SEC61A1, PDK1, CSHL1, PYCR3, CRB1, |
| | PDIA4, CNKSR1, WWTR1, IGLV@, IGLV3-25, PPCDC, IGLL3P, HIST1H2BC, |
| | PPIB, CHAC1, KCNN3, HSPA13, MYDGF, ITGA6, ASIC1, LSR, IGHV1-69, |
| | CKAP4, SDF2L1, TSHR, UAP1, IGKV4-1, GSC2, IGKV1OR2-108, CENPE, |
| | WIPI1, TMEM184B, IGLV4-60, SIL1, LIME1, IGHV3-73, PAK5, IGK, CITED2, |
| | GAS6, SDC1, CADM1, PRDM1, PGM3, GPLD1, IGHG1, SLAMF7, XBP1, |
| | SEC24D, IGKV1-5, HSP90B1, MANEA, MAN1A1, IGF1, TIMP2, IGHM, |
| | SEC14L1, MAST1, DNAJC3, CD59, RRBP1, IGHD, IGLV3-10, SKAP2, IGHV3-72, |
| | VEGFA, MYO1D, NUCB2, IGLJ3, ELL2, HPGD, CAV1, MZB1, AQP3, |
| | TNFRSF17, NT5DC2 |
| Tonsil Unique | CD24, GIT2, RPS21, TNPO3, IL7, ITPR1, LTB, BBS7, ACAP1, GEMIN4, |
| Filtered Down | DCTPP1, KNOP1, RBM12, CNPY3, PSMD11, SZRD1, PMS2P2, VILL, C10orf2, |
| | BACE2, H2AFY, MLLT10, RANBP10, NUP188, TRAF5, NHP2P2, TNFSF11, |
| | NCF2, GPM6A, MPP1, KIAA0226L, COTL1, NVL, DTX2P1-UPK3BP1- |
| | PMS2P11, NUP155, NASP, MBNL3, GALNT12, SNX10, SNRPF, METTL2B, |
| | EIF4E2, LARS2, STAG3, PIN1, CXorf57, USP39, ZCCHC4, RRP9, PAX1, |
| | NLE1, COPS7B, POLD3, DHX8, COX6CP1, BRWD1, CCNF, DCK, RWDD2B, |
| | PPP4C, NUP88, RPUSD2, BHLHE40, TSSC4, POLR3K, MSH2, SIDT1, |
| | ZNF652, RARA, TCL1A, NR4A2, VPS37B, GPSM2, MME |
| Tonsil Unique | KCNG1, GNA11, PPP1R13L, MOCS1, TMEM120B, CNTNAP1, KCTD15, |
| | ADGRF5, MAP3K10, METTL21B, SFN, KIF5A, CAMKV, DNAAF2, PAOX, |
| Filtered UP | AVPI1, PNOC, MTDH, DHDDS, PRR36, EHD2, FUT1, RET, EPOR, PPFIA3, |
| | CRK, CCKBR, F7, CEMP1, NBR1, KAT6B, FGF3, GNG7, FURIN, GNAT2, |
| | OR2S2, GSTM5, PMCH, FICD, NPR1, MAN1A2, CD79A, NR2F6, RAB6A, |
| | NKX3-1, ELF3, TRIP11, GFOD2, USO1, NELL2, ELP6, FAM63A, TRIM10, |
| | OR1F2P, PCDHB11, SLC17A7, GPR12, ERN1, LAMA4, LBP, GYG2, |
| | CDC42EP4, EVC, ARMCX3, DNAH6, DPM3, STS, MAGI2, AGA, PIP5K1A, |
| | TMEM74B, MBP, TMEM57, ROS1, MAPKAPK2, GRIK2, ZNF133, TEAD4, |
| | CETP, TTTY14, AMELY, MRC2, ETNK2, PLOD2, NFKBIB, NLRP3, |
| | SLC22A14, BPIFA1, COX6A2, DPP4, CLEC1A, TRIP10, AGR2, PPP6R2, |
| | VAT1, RAB3B, ABCC3, GOLGA5, SFTPB, NTN1, CUTA, GNAO1, TRADD, |
| | EGR4, ABCG4, CHST4, CEACAM7, RAB26, ADGRL3, KLF1, C1orf116, |
| | CTTN, ABHD2, PGPEP1, TMEM8A, REM1, C21orf2, APOBEC2, CNTN2, |
| | DOCK3, FOXN1, ARFGAP3, LOC101927051, BAIAP2, C9orf116, PARM1, |
| | KLK2, RHO, TRIM2, MPP3, PRB1, CRYBB1, RSG1, RAB6B, APBA2, MPP2, |
| | B4GAT1, CALB2, BSN, COL11A2, UBA5, CNNM1, PRF1, RNF126P1, |
| | DNAJB5, FUZ, FSHR, RPL10, PHTF1, WT1-AS, GJA4, MCAM, GPR31, |
| | CCHCR1, ARHGEF12, TRIM29, GZMA, TRIM15, REEP2, CST2, SPTBN5, |
| | CBX4, CFAP69, ARVCF, ALOX12B, C1orf61, EPHB1, OS9, TTC21B, LRPAP1, |
| | KCNK1, SH3D21, PRM2, TM9SF2, KLK6, LRRN2, OR7E12P, OVOL3, ALX1, |
| | ARHGEF17, GATA2, IMPAD1, PYY2, GP1BA, CHRM2, GFRA2, POLR2C, |
| | SLC13A2, MAP2, ITSN1, DPYS, FTSJ1, EPN2, SRPR, RAB2A, ACTL6B, |
| | SPAG11A, ACE2, PHLDA1, MOGS, SYT13, SH2D4A, PDE11A, DKKL1, |
| | HOXD10, P2RY4, SRP54, NQO1, ASB9, DSPP, FAM153A, STMN2, CRMP1, |
| | PSMB2, NXPH3, CDKN2A, SERPINI1, RAB40C, CYP4F3, P2RX4, AHSP, |
| | VIPR2, CYP2U1, GJB3, PLXNA3, RPL23AP53, STEAP3, TLX1, S100G, |
| | POLR2A, WFDC1, CLTCL1, LSS, YIF1A, OLIG2, ZNF706, TGM4, KLRD1, |
| | C1QTNF1, F2RL1, COL13A1, OPN1SW, ARHGEF15, NCOR2, HNF1B, BIRC7, |
| | PAK6, CACNA1B, STARD8, CACFD1, RAMP2, SIX5, CEP70, CLDN6, |
| | ACADS, MPC1, SMO, JPH2, GOLGA1, RBKS, NMB, IGF2-AS, TM9SF4, |
| | AMBP, TAF4, MAST2, DCC, SFTPD, SYNM, SOX10, HIST1H4H, SNTB1, |
| | FAM3A, PPY, KCNA5, TTC23, SOAT2, PHLDB1, FZD8, GNRH2, OSR2, |
| | SLC39A7, CFTR, MAGEA1, TM4SF5, COMMD3, CHRD, ASCL3, IQCA1, |
| | ALDH3A2, RHD, RNF113A, MEPE, NTRK3, CELA3B, KLK10, HRH3, LY6D, |
| | RHOB, ITPK1, RUSC2, PAK4, GUSB, C9orf16, AQP5, ADGRG1, NPEPL1, |
| | ARMCX4, LENEP, OCRL, HYI, CYP2C19, TRPC4, GPD1, FGF5, TRPC3, |
| | CTBS, BCKDK, NPAS2, HOXA6, TEX40, SEMA3F, CEACAM4, XCR1, |
| | CHST8, ZNF556, PNLIPRP2, TNNI3, DTX3, PIGK, PAX2, HLCS, ZNF574, |
| | HDAC11, TBX6, KSR1, RALGPS1, SEMA5A, OR7A5, ST14, IRS4, SLC9A2, |
| | VWA1, SPINT3, MMP14, POFUT2, SDF4, SCAMP5, BTG4, MCF2, IMPG1, |
| | PLA2G16, CDH2, MAPK8IP2, CHPF2, CNTD2, PGLYRP1, ASIC4, SLCO5A1, |
| | GLT8D1, PKP1, C10orf95, NEU3, AGTR2, AARS, CUX2, SEZ6L, GZMB, |
| | CYLC1, IFNA4, CREB3L1, AMPH, ALDH6A1, FCN2, ADH6, COX7A1, |
| | ITGA8, CASR, CROCCP3, CX3CL1, B3GNT4, NUPR1, UBTD1, CEACAM3, |
| | CHRNB3, LHX6, CACNA1F, NEDD9, HRASLS2, KCNK2, FAM163A, GFPT1, |
| | THBD, BEST2, MUC1, ID1, KCNJ4, SERPIND1, ALDOAP2, LTK, FOXN3-AS2, |
| | AHI1, ECEL1, ADAMTS2, PDE1B, PRKCG, ST6GAL1, TMEM59, DLX5, |
| | BCL9, THEG, COL14A1, TTC39A, ARMC9, NRXN3, GP2, S100A3, ZNF609, |
| | C7orf69, C1S, KCNS1, GABBR2, GPR161, ATP2B4, SLC9A3R2, SLC12A4, |
| | AMPD1, HAND2, LOC643733, TNFRSF11B, TRPM6, GEMIN7, FAM107A, |
| | CTSG, HIPK2, ELL, SLC6A8, CEP250, KCNMB2, CPNE7, FAM69A, FBXW7, |
| | NOP16, MMP19, HTR5A, GABRR2, SBNO2, CDK18, NXPE3, DNAI2, GALR1, |
| | IFNA6, SYTL2, WFS1, RGS12, OMD, CD5L, FABP3, SH3GL2, FOXL1, |
| | ADORA2B, UFSP2, PDGFB, LOC105372602, DLEC1, PICK1, KCNQ1DN, |
| | SSX3, DHRS9, MYL10, TMEM104, RIPK4, HN1L, PALM, PKNOX2, RLBP1, |
| | PMFBP1, EVI5, ZFHX2, CACNG4, SMAD6, DGCR14, PADI4, ZNF408, RGS6, |
| | SSTR3, TRH, RUNX2, ABCB9, KLF5, HSPG2, ATP6V1B1, SLC12A5, FGFBP1, |
| | LRP6, APOBEC3B, FAM110B, MAGOH2P, NR1H4, DTNB, ARID3A, DES, |
| | GIPC2, KCNJ1, CMA1, NRP1, IGFBP5, PLD3, ROR2, NAT8B, KIFC3, BMP1, |
| | MMACHC, C1QL1, NKX3-2, CCDC170, MYO5A, CEACAM5, CDH8, SYDE1, |
| | MSLN, COPG1, KCNK3, ICAM2, PYY, MFAP5, SLC29A1, FUT3, TFAP2A, |
| | HDC, ZNF440, CRISP1, ROM1, SEMG1, APOBEC3F, OSBPL3, HR, GOLIM4, |
| | ABHD14A, FCAR, TAAR5, MAGEA11, PARD3, LLGL1, NPC2, FGFR2, EDN2, |
| | ZNF334, CPB2, GH2, AVEN, PAMR1, SRCAP, PPY2P, EPS8L2, STEAP4, |
| | SLC48A1, PAX8, MAPK4, NUDT2, PRDM14, NXPE4, KDELR1, KLHL4, |
| | HCRTR1, STAR, STX11, ETV4, TCF3, BRAP, RNASE4, HOXA3, ACBD4, |
| | TAPBPL, KRT18, KCNJ8, STAB2, OSBPL1A, AKAP5, PRLR, EVPL, MTUS1, |
| | RUNDC3A, PCDHB13, SMOX, FASN, CTIF, TINAGL1, SMPD1, ACAA2, |
| | DGCR9, ARSF, BHLHB9, SLC7A10, USP33, RHAG, LOC79160, FCGR1A, |
| | ZNHIT2, METRN, CASP10, ACE, ALX3, CDK5R2, CRX, WBP5, SSX2B, |
| | NMUR1, FZD4, DAPK1, SCUBE2, ATP2B3, MTHFR, SIX1, PIM2, KCNH6, |
| | GLI2, SOCS7, CTDSPL, ABCA3, ADRA2C, GTF2A1, EVA1B, LGALSL, TSKS, |
| | SIGLEC8, A4GALT, AAAS, NENF, C19orf73, TMEM45A, CHIT1, RAB11B, |
| | PKLR, RABAC1, CALCA, AXL, HIST1H2BI, EPHB3, CNIH3, TRIM17, |
| | PRSS16, C16orf45, PTP4A3, COL1A2, PIGO, DNAJB4, ADRBK1, TJP1, TFF1, |
| | DEXI, AREG, POU3F1, NOL3, ALKBH4, IGFBP3, SEMA3B, FHL5, AIF1, |
| | TFAP2B, HIST1H2BF, TRAPPC9, RNF5, MYH14, CYB561, BET1L, ITGB3, |
| | RBMXL2, CYP3A7, SLC5A7, OR2J2, ZKSCAN1, EPO, MPL, LRRC23, |
| | BTN1A1, BMP7, IQCE, HIST1H2BE, LOC730101, ANXA13, CCL25, OPCML, |
| | CTSV, ACOXL, FAM149B1, CGREF1, CADPS2, NMRK1, OR2F1, NNMT, |
| | CELF3, PLAC4, TJP3, P2RX1, RHCG, PLXNA2, ABLIM3, NR0B1, ACADL, |
| | GRIN1, SLC22A17, TRO, F12, LAMC1, RAPGEF4, GYPB, NCAM1, DUSP26, |
| | TAL1, NCR1, ACACB, FBP2, SEPT5-GP1BB, PVRL1, PDGFA, HOXB9, |
| | GRIP1, SOX15, DDX28, LOC101929272, KDELR3, ABCC4, GLRA2, CYTL1, |
| | IL17RB, VPS45, MTMR8, ALDH1A2, DNAJC12, ACOT11, PLXNB3, ERO1B, |
| | FER1L4, C1QTNF9B-AS1, NR0B2, SPARCL1, TLR8, CDH15, DKK4, SSTR2, |
| | SLC7A8, ADGRD2, PLCE1, SLC2A10, ARSA, CRTC1, GRB14, GABARAPL1, |
| | SERPINB13, LTC4S, MROH9, SORBS1, LOC440792, PAX7, CREB3L2, |
| | SLC38A4, CCPG1, POU3F2, RNF17, RPARP-AS1, P2RY6, HAB1, HEYL, |
| | CD46, KCNQ1OT1, CIDEA, GULP1, QPCT, PTOV1-AS2, LRP2, GSDMB, |
| | PCYT1B, HIST1H3H, TMEM259, GALR2, FOXRED2, PDZD7, APOL5, ECRP, |
| | PYGO1, MDK, IL4, COBLL1, HIST1H2AG, IGF1R, MAG, GUCA1A, H1FX, |
| | SH3BGR, ERG, FOXJ1, SOX14, KIAA1024, SPTB, MYH11, FZD6, PLPPR2, |
| | KIZ, PPP1R26, SMPDL3B, ROBO3, PTGIS, GLP1R, PTGDR2, GREB1, NDST1, |
| | EHHADH, NOL4, NPHS2, NOS1, IL1RL1, ZMYND10, DNAJB9, APOA1, |
| | CBARP, WNT10B, LRIT1, VPREB1, KIF1C, HBB, ACR, AOAH, PTGER3, |
| | KRT20, COL7A1, FNDC3A, FEZ1, PLEKHG3, TEK, KCNH1, PSEN2, |
| | HIST1H1C, PRX, CCT8L2, GKN1, ATXN8OS, VNN1, GPER1, UGT2B15, |
| | DNASE1L2, KANK2, MYL7, GRP, HIST1H2AE, BCL2L1, SATB2, SFTPC, |
| | SPAG4, EPN1, TTC38, BMP5, NUP62CL, MROH7, C1R, PLTP, CDX1, SSPN, |
| | NRXN2, LRRC2, LOC55338, CALML5, HIST1H2BO, RAB40B, LMF1, NPTX2, |
| | TSPAN1, MAP3K13, KCNA3, CDH4, MSX2, CPD, ANKRD36BP2, CECR1, |
| | ACSM5, TBX2, CTGF, PECAM1, GAS1, DDN, TNFRSF4, IGHV3-47, CD9, |
| | CABP1, WNT5B, C10orf10, TSPAN12, DNAAF1, CDH1, FRZB, TRPM4 |

Table 71G provides a list of single-cell RNA-Seq (scRNA-Seq) clusters, and sets of genes associated with disease activity in SLE, which were identified using methods and systems of the present disclosure as being strongly correlative with each given scRNA-Seq cluster. These sets of genes can be used as effective SLE biomarkers to indicate disease activity via the given scRNA-Seq clusters.

TABLE 71G
Single-Cell RNA-Seq

| scRNA-Seq | |
|---|---|
| Cluster | Genes |
| DC1_genes | CLEC9A, C1ORF54, HLA-DPA1, CADM1, CAMK2D, CPVL, HLA- |
| | DPB2, WDFY4, CPNE3, IDO1, HLA-DPB1, LOC645638, HLA-DOB, |
| | HLA-DQB1, HLA-DQB, CLNK, CSRP1, SNX3, ZNF366, KIAA1598, |
| | NDRG2, ENPP1, RGS10, AX747832, CYB5R3, ID2, XCR1, FAM190A, |
| | ASAP1, SLAMF8, CD59, DHRS3, GCET2, FNBP1, TMEM14A, NET1, |
| | BTLA, BCL6, FLT3, ADAM28, SLAMF7, BATF3, LGALS2, VAC14, |
| | PPA1, APOL3, C1ORF21, CCND1, ANPEP, ELOVL5, NCALD, ACTN1, |
| | PIK3CB, HAVCR2, GYPC, TLR10, ASB2, KIF16B, LRRC18, DST, |
| | DENND1B, DNASE1L3, SLC24A4, VAV3, THBD, NAV1, GSTM4, |
| | TRERF1, B3GNT7, LACC1, LMNA, PTK2, IDO2, MTERFD3, CD93, |
| | DPP4, SLC9A9, FCRL6, PDLIM7, CYP2E1, PDE4DIP, LIMA1, |
| | CTTNBP2NL, PPM1M, OSBPL3, PLCD1, CD38, EHD4, ACSS2, |
| | LOC541471, FUCA1, SNX22, APOL1, DUSP10, FAM160A2, INF2, |
| | DUSP2, PALM2, RAB11FIP4, DSE, FAM135A, KCNK6, PPM1H, |
| | PAFAH1B3, PDLIM1, TGM2, SCARF1, CD40, STX3, WHAMMP3, |
| | PRELID2, PQLC2 |
| DC2_genes | CD1C, FCER1A, CLEC10A, ADAM8, CD1D, FCGR2B, CLEC4A, |
| | SLC2A3, CD33, ETS2, CLIC2, PEA15, CACNA2D3, CD1E, MBOAT7, |
| | C10ORF128, NR4A2, AGPAT9, ENTPD1, CD2, PER1, PID1, AREG, |
| | PTGS1, SMA, CLEC17A, ITGA5, CREB5, PTAFR, NOD2, CCR6 |
| DC3_genes | S100A9, S100A8, VCAN, LYZ, ANXA1, PLBD1, RNASE2, FCER1A, |
| | SLC2A3, CD163, CSF3R, MNDA, CD14, NAIP, CSTA, FCN1, CD1D, |
| | FPR1, F13A1, CLEC10A, CES1, PID1, S100A12, MTMR11, SMA, |
| | LAT2, RETN, TMEM173, AOAH, RAB3D, CD36, MGST1, TREM1, |
| | HNMT, CES1P1, ADAM15, IL13RA1, MICAL2, ITGA5, CREB5, IL1B, |
| | NR4A2, MPP7, PTAFR, HBEGF, NFE2, ASGR1, BST1, IL1RN, NOD2, |
| | NLRP3, DQ575504, LMNA, C9ORF89, IL27RA, NLRP12, RAB27A, |
| | EREG, LOC284454 |
| DC4_genes | FCGR3A, FTL, SERPINA1, LST1, AIF1, SAT1, CTSS, MTSS1, TCF7L2, |
| | AK307192, PSAP, FTH1, IFITM3, MS4A7, LILRB2, PILRA, CSF1R, |
| | ASAH1, LRRC25, HLA-E, IFITM2, LYST, HCK, C5AR1, WARS, |
| | PECAM1, CTSL1, S100A11, CFD, HK3, MAFB, TNFRSF1B, DUSP6, |
| | CASP1, SIGLEC10, FGR, SLC7A7, BIN2, LILRA2, SIDT2, NEAT1, |
| | PTPN6, RHOC, SLC11A1, LOC200772, TYROBP, IFI30, EMR2, |
| | GIMAP4, DUSP1, TNFSF10, GBP2, FAM110A, LY6E, TXNIP, |
| | TSC22D3, HMOX1, CD68, CD52, TBXAS1, TMEM176B, C10ORF54, |
| | S100A4, BCL2A1, CD97, PTPRC, FAM26F, FCN1, ITGAL, OAS1, FYB, |
| | ABI3, ITM2B, LILRA6, TSPAN14, CD79B, LILRA5, SLC31A2, |
| | NFKBIZ, LILRB1, FCGR3B, CD300LF, SOD2, CLEC7A, MYO1G, |
| | NAMPT, CX3CR1, RAP1B, MSN, FCGR2C, RAB24, GLUL, GPBAR1, |
| | CHST15, CPPED1, CDKN1C, TAGLN, TKT, BID, NCF2, SMAP2, |
| | CD300E, EMR1, TIMP1, PTP4A3, VMP1, NINJ1, POU2F2, GNS, |
| | RNF144B, ICAM2, STX11, STXBP2, FLNA, NEURL, PIK3AP1, |
| | SH2D1B, MARCKS, SLC44A2, TUBA1A, DPEP2, CXCL16, HSPA7, |
| | SSH2, FCGR2A, C3AR1, DRAP1, CYTIP, RXRA, LYN, NAP1L1, IFIT3, |
| | IFITM1, NAAA, CD300A, DOK3, CALML4, NADK, PHTF2, TESC, |
| | MS4A4A, ALOX5, PAG1, SDCBP, MT2A, P2RX1, ZEB2, ARAP1, |
| | DOK2, HSBP1, LGALS3, TTYH3, C19ORF38, WSB1, CLEC4F, GBP4, |
| | HK1, IRAK3, BLVRA, ATP1B3, RNF149, TCIRG1, PRAM1, SPN, |
| | ZCCHC6, CLEC12A, CNIH4, IFI6, MAP3K1, INSIG1, SLC2A6, |
| | DMXL2, AK124399, ALDH3B1, TLR4, C11ORF21, C20ORF112, CKB, |
| | NPL, NDUFB3, RAB10, TMC6, ICAM4, DNASE2, C9ORF72, GIMAP7, |
| | KLF3, DKFZP451J181, TIAM1, CDC42EP3, STK10, TLR2, AGTRAP, |
| | APOL6, CDH23, FPR1, AL137655, VAMP5, IRF1, SH3BP2, YPEL2, |
| | GRAMD1A, ISG15, LRP1, MXD3, AMPD2, CD244, GBP1, LCP2, |
| | ZFAND5, HEG1, LOC388312, ARRB1, FAM46A, ABCC3, GBP5, SVIL, |
| | ARRB2, FAM45B, LTA4H, NFAM1, CSK, TBC1D8, GNG2, MYOF, |
| | RAB37, VPS53, APOBEC3A, ITGB1, P2RY13, C15ORF39, DENND5A, |
| | NBEAL2, PLIN2, PIK3IP1, SCIMP, TMPO, KIAA0513, C10ORF46, |
| | CASP4, FGD4, IFNGR2, PTGER2, SAMSN1, UBXN11, TBCD, VASP, |
| | CCM2, NLRP1, GIMAP1, NR4A1, TNFRSF14, MBD2, SCPEP1, |
| | DENND3, IFIT2, NECAP2, PTGER4, RASGRP4, TMBIM1, SIRPB1, |
| | STK38, EVL, GIMAP2, LIMS1, FGD3, SLA, SULT1A1, WDR11, |
| | PSTPIP2, PDLIM5, RALB, ABHD3, ARRDC3, KLF11, TMTC1, |
| | RAP1GAP2, SNX18, RAB3D, ADRBK1, ARHGEF3, BACH1, DDX60, |
| | PIEZO1, CMTM7, IMPDH1, TSPAN32, DDX58, CCPG1, DDX60L, |
| | PNPLA6, UNC13D, SYTL1, CSGALNACT2, TLE4, SIRPB2, UPP1, |
| | ARAP2, ERICH1, PPM1F, GPR155, MPP1, PELI1, TMEM154, L1TD1, |
| | WDFY1, FOXO1, PLXNC1, MCTP1, AP2A1, DNAJB1, SWAP70, |
| | TMEM11, TMEM134, CABP4, LOC100133161, ARL4A, EHD1, ACOT9, |
| | KSR1, KDM1B, PDP1, AK123771, PITPNM1, FAM126A, MAGED2, |
| | CAMK1, IL12RB1, PYGL, CORO2A, ZNFX1, TYMP, NUDT16, SGPL1, |
| | MEFV, RELT, PTPN13, FCAR, SASH1, PLEKHO2, BLOC1S3, |
| | CAMKK2 |
| DC5_genes | AXL, PPP1R14A, SIGLEC6, CD22, DAB2, S100A10, FAM105A, |
| | MED12L, ALDH2, LTK, DPYSL2, LGMN, IRF4, SEPT6′, PLAC8, |
| | CCND3, MYO1E, SLC41A2, SCN9A, SIGLEC1, CX3CR1, NDRG1, |
| | VASH1, CD5, BHLHE40, SNRNP25, USF2, SLC20A1, ATF5, |
| | FAM129A, KLF4, RUNX2, ARHGAP18, APEX1, ENTPD7, SLC35C2, |
| | CDH1, GPR146, BAIAP2, CDKN1A, UPK3A, GNAQ, THBD, TNFSF12, |
| | SOX4, CXCR2, HIP1, STX18, CTSW, ATP2B4, CD72, MGLL, SUSD1, |
| | RNF141, TNNI2, GGTA1P, C5ORF25, PTGDS, TSEN54, KLF12, |
| | MYH11, TXN, AK125727, CD300LB, SUCLA2, BIN1, MRPS6, ZNF789, |
| | RAD1, PIM2, PLA2G16, TBC1D9, ADAM33, ZEB1, CD300LG, |
| | SLC4A3, STAG3L4, MECR, COQ7, RBL1, CEP95, RNASEL, ACPP, |
| | SP4, LAX1 |
| DC6_genes | GZMB, IGJ, AK128525, SERPINF1, ITM2C, PLD4, CCDC50, IRF7, |
| | PTPRS, ALOX5AP, TCF4, BCL11A, LILRA4, PLAC8, C12ORF75, |
| | FAM129C, CYBASC3, MZB1, UGCG, DERL3, IL3RA, SPIB, ZFAT, |
| | SMPD3, NRP1, TSPAN13, LIME1, CLEC4C, CLIC3, SPCS1, NPC1, |
| | HIGD1A, CTSB, NPC2, SEC61B, C1ORF186, TNFRSF21, IRF8, |
| | HERPUD1, PLP2, SLC15A4, CD164, BLNK, NCF1C, HSP90B1, OGT, |
| | SELS, IRF4, APP, TXN, RUNX2, PTPRCAP, GPR114, STMN1, |
| | RNASE6, PFKFB2, MAP1A, NUCB2, SSR4, LAMP5, NCF1, B4GALT1, |
| | IGFLR1, NOTCH4, GPR183, EPHB1, LOC285972, MYBL2, PTCRA, |
| | SLA2, AK093551, PLXNA4, SEPT1′, C10ORF118, LILRB4, GAPT, |
| | IDH3A, MS4A6A, FMNL3, SNRPN, KIAA0226L, BC051760, |
| | ST6GALNAC4, OFD1, C9ORF142, TGFBI, SELL, SIDT1, TRAF4, DCK, |
| | ERN1, TPM2, PARK7, TLR7, CARD11, DAB2, ERP29, PACSIN1, |
| | LOC644961, RABGAP1L, ADAM19, SORL1, PPP1R14B, SCAMP5, |
| | USP24, ZDHHC17, CXCR3, MAN2B1, RNASET2, FCHSD2, LAIR1, |
| | OVOS2, P2RY14, CYTH4, PPM1K, ABHD15, EIF4A3, P4HB, NCF1B, |
| | TSPAN3, TRAM1, ABPARTS, COBLL1, CREB3L2, TMEM109, |
| | SCN9A, CYP46A1, LGMN, NGLY1, C17ORF109, PLA2G16, SLC38A1, |
| | PHEX, CD99, PPM1J, C10ORF58, KIAA0226, DHRS7, CNP, CDCA7L, |
| | SIT1, TACC1, RASD1, TMIGD2, KRT5, ASPH, LOC652276, PDIA4, |
| | AHI1, GPM6B, HPS4, SIVA1, LOC100507600, UBE2J1, FAM160A1, |
| | IFI44L, MAPKAPK2, CMKLR1, AX747844, GGA2, TP53I13, CSF2RB, |
| | LOC100233209, TCL1A, ATP2A3, FLNB, NEK8, TBC1D4, CUX2, |
| | PDCD4, SND1, SLC2A1, SMC6, LY9, STAMBPL1, KIRREL3, |
| | SCARB2, EMB, PAFAH2, VEGFB, AL833181, DQ572107, ZCCHC11, |
| | DUSP5, SLC38A2, SLC7A5, TTC24, ANKRD36, TMEM19, |
| | LOC100131564, CD2AP, GAS6, IGFBP3, MIF4GD, IRF2BP2, CRYM, |
| | DKFZP586I1420, DKFZP667P0924, TEX2, FLJ43663, FKBP2, SPICE1, |
| | AHNAK2, ANKRD36BP1, RNF5, RRBP1, SLC12A3, SLC3A2, SEC61G, |
| | ATP13A2, LRRC36, AK095700, C12ORF44, POLB, LMAN1, |
| | AK057596, PHC3, SUSD1, ANKRD36B, CRIM1, MGAT4A, SEL1L3, |
| | SLC7A11, MILR1, PAPLN, CLN8, VAMP1, CCDC69, KANK1, LTB, |
| | STRBP, SLC20A1, SNURF-SNRPN, SOLH, PARP10, BX647938, PAIP1, |
| | MAGED1, DHTKD1, IL28RA, C5ORF62, SLC35E2, FZD3, EGLN3, |
| | MEF2D, TNFAIP3, COL24A1, MCOLN2, TUBB6, CLCN5, FUT7, |
| | SFT2D2, CSNK1E, NOP56, ST3GAL4, DPPA4, GNG7, SEC61A1, |
| | DSN1, FLJ42627, ZDHHC4, CCR2, C6ORF25, ITPR2, TMEM63A, |
| | ABCA2, ADA, FOXRED2, ST3GAL2, PMS2P5, SGSM3, USP11, GAB1, |
| | STT3A, SULF2, C18ORF8, DENND5B, NFX1, SUZ12P, CTNS, |
| | TXNDC5, SETBP1, TATDN3, LOC642776, MDFIC, SEC11C, UBA5, |
| | MYO1E, TASP1, PIK3CD, MDN1, PPARA, DQ576756, TCL6, TGFBR2, |
| | TP53I11, 11-Sep, SBDS, ZFYVE26, BTAF1, C5ORF45, PTK7, SRPR, |
| | ERO1LB, NAPSA, C9ORF91, STAG3L3, TULP4, CYSLTR1, |
| | LOC284551, SNRNP25, ALG2, ITGAE, MAP2K6, TBCC, OCLN, DCPS, |
| | LRP8, STAG3L1, KRR1, C12ORF45, PCYOX1, SPNS3, TPST2, MYB, |
| | SLC12A2, ZBTB33, ABI2, PMS2L2, GLCE, ITPR1, MRPL36, C5ORF64, |
| | PFKP, S100PBP, SPON2, SPG20, TRDMT1, N4BP2L1, PPP6R1, RCL1, |
| | ZNF506, AHCY, CXORF21, CCS, RNASEH2B, SYS1, P2RY6, PPFIBP1, |
| | NFATC2IP, ZNF527, MINA, TAX1BP3, DAAM1, GALNT2, |
| | LOC400657, C1ORF55, RREB1, VIPR2, ARL6IP6, QDPR, ABCA7, |
| | SLC23A2, BEX4, SLC33A1, THSD1P1, ARHGEF4, C6ORF170, N4BP2, |
| | SPATA5, CRYM-AS1, IQGAP2, DAPK2, MFSD2A, PCMTD1, ANKS3, |
| | CEP135, LOC100131089, ALDH5A1, BC034268, MAP4K4, SERTAD2, |
| | PCNX, PHLPP2, EFHC1, SP4, TRRAP, NICN1, TRIM74, HNRNPA1L2 |
| Mono1 | CD14, VCAN, S100A8, S100A9, FCN1, ITGB2, LRP1, CSF3R, TKT, |
| | LYZ, APLP2, FPR1, CD36, S100A12, CLEC4E, ITGAM, SLC2A3, |
| | CTSD, NEAT1, PTAFR, TREM1, NAIP, NCF1, FCGR2A, SCPEP1, |
| | CTSA, NLRP3, ACSL1, SDCBP, SLC11A1, IRS2, VNN2, DPYD, |
| | CLEC7A, BST1, PLBD1, PYGL, QPCT, BC013828, CD163, AQP9, |
| | PELI1, FAM198B, GAS7, STAB1, CDA, DOK3, IRAK3, PLAUR, |
| | AL137655, LILRA6, TLR4, AX747598, TLR2, AGTRAP, CRISPLD2, |
| | CCR1, NFAM1, ETS2, RAB27A, BNIP3L, HPSE, PER1, MEGF9, |
| | CD300E, CYP1B1, FCAR, SOD2, UPP1, IER3, C5AR1, NLRP12, SMA, |
| | DMXL2, NCF1B, CREB5, CR1, ALDH1A1, ASGR1, FNDC3B, DUSP6, |
| | TOM1, CDC42EP3, ZBTB16, DYSF, KCNE3, CD93, CEBPD, FCGR1A, |
| | PLEKHM1, CPM, MPP7, AK302511, IL1B, PFKFB3, PLD3, SMA3, |
| | F13A1, G0S2, LOC100133161, PHF21A, TLR8, CLMN, TNFAIP3 |
| Mono2 | LAIR2, ASAH1, APOBEC3A, TSPAN14, LIPA, CYTIP, SIGLEC10, |
| | LILRB1, EMR1, TTYH3, CAMKK2, CX3CR1, C3AR1, BC013828, |
| | RASGEF1B, BIRC3, PLIN2, CD300C, CD83, XYLT1, KLF2, FBP1 |
| Mono3 | G0S2, NAMPT, NEAT1, AL137655, CSF3R, FCGR3B, SRGN, TREM1, |
| | TNFRSF10C, MXD1, SOD2, CXCR2, SLC25A37, S100A8, FPR1, |
| | ITM2B, MNDA, VNN2, SDCBP, CXCR1, S100A9, AQP9, SORL1, |
| | ACSL1, AX747598, R3HDM4, NCF1, IFITM2, FCGR2A, XPO6, GCA, |
| | C5AR1, TKT, PELI1, SLC2A3, CLEC4E, MMP25, GLUL, CD14, |
| | LOC388312, NCF1C, VMP1, RTN3, ACTN1, PTAFR, S100A12, |
| | SEC14L1, DQ574721, LITAF, TLR2, SHKBP1, LIMK2, LOC100505702, |
| | PYGL, RNF24, DNAJC25-GNG10, IL8, FPR2, LOC731275, SLC12A6, |
| | IL1R2, VNN3, CFD, VCAN, BC013828, NAIP, ZBTB16, BCL2A1, |
| | FAM129A, PLAUR, FNDC3B, FP15737, SEPX1, LOC100133161, PER1, |
| | FBXL5, IL17RA, TLR4, IGF2R, ITGAM, HIST1H2AC, LRP1, |
| | KREMEN1, C12ORF35, PRRG4, CR1, RAB27A, LOC100505815, BST1, |
| | NUMB, USP15, CDA, IER3, ACADSB, DYSF, PXN, PDP2, TNFRSF1A, |
| | LRG1, LOC91948, FLJ45445, SMAP2, LOC643802, NINJ1, ABTB1, |
| | CCNY, TMEM154, CCR1, CARD8, TACC3, TMEM71, PTGS2, HPSE, |
| | C3ORF72, FAM157A, AK130076, CD163, NBEAL2, IL1RAP, GK, |
| | AZGP1P1, DOK3, PROK2, FAM115C, QPCT, ALPL, BEST1, CES3, |
| | CREB5, SPAG9, GPR97, TBL1X, FAM198B, FCAR, PHF21A, IRS2, |
| | CYP1B1, NCF1B, BC048113, BACH1, AX747405, RCBTB2, CEBPD, |
| | ALPK1, LAT2, OSBPL8, PCNX, LPPR2, CCPG1, DOCK5, TUBA4A, |
| | F2RL3, NCF4, FAM157B, TECPR2, SLA, TM6SF1, CRISPLD2, FAS, |
| | PADI4, RUFY1, AK302511, PDE4B, AK091866, DQ580909, FAM126B, |
| | LRP10, PADI2, TRIBI, ZDHHC18, F5, PDLIM7, RBM47, SIRPA, |
| | ARHGAP26, DSTYK, TLR6, FBXL13, LOC649305, P2RY8, HBP1, |
| | SGSM1, ABCA1, SEMA4D, ABHD5, MRS2P2 |
| Mono4 | PRF1, GNLY, KLRC4-KLRK1, TCRBV3S1, CTSW, CCL5, KLRD1, |
| | FGFBP2, NKG7, IL2RB, SPON2, HOPX, GZMA, CST7, ZAP70, GPR56, |
| | SYNE2, KLRF1, GZMH, IL32, TXK, IFITM1, IKZF3, LCK, TC2N, |
| | S1PR5, S100A8, MCTP2, S100A12, CD96, SAMD3, TRGC2, TTC38, |
| | PXN, S100A9, SH2D1B, LAIR2, SYNE1, PRKCH, RARRES3, PIK3R1, |
| | CCL4, PARP8, TGFBR3, GSTM1, CD2, CD247, PDE4D, PRDM1, |
| | CBLB, GIMAP1, BC013828, DENND2D, GZMM, SKAP1, TMEM41A, |
| | KLRB1, PLEKHG3, FCRL6, PYHIN1, AAK1, CCR1, IRS2, STAT4, |
| | IL18RAP, INADL, DIP2A, LOC388692, FAIM3, CD160, PAPD5, PAM, |
| | PIK3IP1, PRSS23, PVRIG, VNN2, CREB5, CCND2, RORA, ATXN7, |
| | PTPN4, LIMK2, SEPX1, KLF12, TRDC, AK094156, NCR3, KIF21B, |
| | PTGDR, IER3, ITK, BTN3A2, CPD, NCAM1, ZBTB16, RAB27A, |
| | RUNX3, SLC25A37, SLFN13, GCA, RASA3, IPCEF1, SCML4, NID1, |
| | PADI4, S1PR1, ZBTB38, FCGR1A, PARP15, ETS1, LAT, TRPM2, |
| | FNDC3B, CCL3, CLEC4D, OPTN, RASSF3, LOC100216546, IL1B, |
| | GBP5, ENC1, KLRG1, SYTL3, BC051736, TRAPPC10, LIN54, |
| | LOC374443, ZNF44, F2R, TFDP2, CEP78, CXCR2, G0S2, |
| | GABARAPL1, TUBD1, PDPR, DQ573668, FXYD6-FXYD2, BRF2, |
| | SLAMF6, CREM, TGIF1, SLFN5, ARHGAP24, ZMYM5, ZNF276, |
| | SUPV3L1, FAM190B, LPIN1 |
| AMP Kid | FCN1, IFI30, AIF1, MAFB, LYZ, CD300E, SERPINA1, APOBEC3A, |
| Monocyte | PSAP, CYBB, S100A8, CST3, C5AR1, CTSS, TYROBP, LST1, MS4A7, |
| Cluster 1 | LRRC25, NCF2, TIMP1, LILRB2, HCK, IFITM3, CD68, S100A9, GRN, |
| | FCER1G, SECTM1, CFP, RP11-290F20.3, IGSF6, FTL, CD14, DMXL2, |
| | COTL1, HMOX1, CFD, TTYH3, CD36, CEBPB, SLC31A2, DUSP6, |
| | TCF7L2, MARCKS, ZNF385A, VCAN, LILRA3, FGL2, C1QA, HLA- |
| | DRA, SPI1, RP11-1143G9.4, EMR2, NPC2, SLC11A1, PLAUR, RRAS, |
| | SIGLEC1, CD74, CSF1R, CXCL16, S100A11, SAT1, SCIMP, CTSB, |
| | C1QB, TNFAIP2, RHOB, FPR1, LRP1, TNFSF13B, MPEG1, MNDA, |
| | WARS, C1orf162, ADAP2, RAB31, IL1RN, PILRA, SLC8A1, AP1S2, |
| | RGL1, MS4A4A, PDK4, RXRA, CLEC7A, PLXNB2, MSR1, TYMP, |
| | MS4A6A, KCTD12, TGFBI, EPB41L3, FCGR3A, CTSL, POU2F2, LYN, |
| | CCDC88A, CCR1, IFNGR2, RGS2, CDKN1C, KLF4, HLA-DRB1, ZEB2, |
| | BRI3, TLR4, EMILIN2, LGALS2, P2RY13, BLVRB, HLA-DRB5, |
| | IRAK3, GPX1, DOK3, FCGRT, HK3, HLA-DPA1, TLR2, CLEC12A, |
| | ANXA5, LMO2, ANPEP, CDKN1A, LTBR, IFI27, LPCAT2, ATP6V1B2, |
| | FAM26F, CD4, SASH1, NAGK, SLC2A6, SLC7A7, PTAFR, TIMP2, |
| | LGALS1, IFIT3, LILRB4, FGD2, CD163, CEBPA, LGALS3, SIRPA, |
| | CSF3R, SOD2, CEBPD, C3AR1, RBM47, SOX4, PPT1, SORT1, |
| | NAMPT, IL1B, CHST15, BCL6, AC104809.4, CPVL, SLC43A2, |
| | SCARB2, MYOF, NFKBIZ, RNF130, CPM, SKAP2, GNS, TMEM176A, |
| | MARCH1, SLC15A3, BTK, ASAH1, C15orf48, CD302, CD86, SPP1, |
| | RASSF4, FMNL2, CD93, MGLL, SAMHD1, CTSD, CYP1B1, APLP2, |
| | LGALS9, EFHD2, LRRK2, LHFPL2, BCL2A1, GLUL, FGD4, |
| | SERPING1, C2, JUP, MTRNR2L2, IFI6, PEA15, RNF144B, HNMT, |
| | NR4A1, FCGR2A, FGR, SIGLEC10, FPR3, ADRBK2, PLEKHO1, LPL, |
| | IL13RA1, ATF5, PLXDC2, APOBEC3B, RNASE6, C1QC, HLA-DMB, |
| | TNFSF10, ANXA2, HLA-DQA1, MYO1E, KIAA1598, HSPA1A, |
| | CSF2RA, IGHG2, CD180, ZNF503, LILRB1, TMEM176B, PRKCD, |
| | CKAP4 |
| AMP Kid | C1QC, C1QA, C1QB, CD14, MSR1, MS4A4A, FPR3, SIGLEC1, CD163, |
| Monocyte | MAFB, C5AR1, CD36, MS4A7, CD68, TNFAIP2, PDK4, SERPING1, |
| Cluster 2 | C2, IGSF6, MS4A6A, GPNMB, GRN, RGL1, CYBB, ADAP2, SLCO2B1, |
| | AIF1, CXCL2, CTSB, ITSN1, IFI30, TGFBI, CTSL, PSAP, TLR4, |
| | SLC1A3, SECTM1, MARCKS, HMOX1, DAB2, BLVRB, KCTD12, |
| | IFI27, CREG1, SASH1, NCF2, CLEC7A, VSIG4, TIMP2, CSF1R, |
| | CXCL3, LILRB4, HNMT, RP11-1143G9.4, TYROBP, ZNF385A, |
| | DMXL2, EMP1, CST3, LRP1, LYZ, LILRB2, CD302, RNASE1, FMNL2, |
| | CD300E, FTL, CFD, FCER1G, PILRA, LGMN, RP11-290F20.3, SIRPA, |
| | SLC31A2, MGLL, EPB41L3, IFITM3, IFNGR2, SERPINA1, TLR2, |
| | ABCA1, LRRC25, IL1RN, CPM, MPEG1, FCGR2A, LST1, CXCL16, |
| | HCK, LTBR, PLAUR, CEBPD, KIAA1598, CFP, ANPEP, EMR2, |
| | TIMP1, C3AR1, EMILIN2, SLC7A7, FOLR2, PTAFR, SLC11A1, CCR1, |
| | CPVL, RNASE6, FCN1, CYFIP1, NRP1, GLUL, CTSS, RNF130, TNS1, |
| | FCGRT, HK3, C1orf162, ADORA3, CLEC12A, ETS2, RBM47, P2RY13, |
| | SAT1, SGK1, LHFPL2, SCARB2, MARCH1, FGL2, CTNND1, NPC2, |
| | FPR1, RHOB, S100A9, RAB31, TTYH3, S100A11, CD86, ZFHX3, |
| | LGALS3BP, ATP1B1, TREM2, CSF3R, STAB1, LGALS3, GSN, MNDA, |
| | BCAT1, FGD4, CDKN1A, SLC15A3, RNF144B, SLC8A1, CEBPB, |
| | CCDC88A, LMNA, GPX1, APOBEC3B, CTSD, CD4, DUSP6, HLA- |
| | DRB5, YBX3, ETV5, NAMPT, CHST15, FNIP2, MFSD1, FCGR3A, |
| | ASAH1, BRI3, ANXA5, ALDH2, MYOF, GPR82, PLXDC2, IFNGR1, |
| | MITF, SPI1, JUP, LMO2, LIPA, COTL1, ACTN1, HLA-DRA, DPYSL2, |
| | CSF2RA, S100A8, ZEB2, HSPA1A, SDCBP, FSCN1, KLF4, TCF7L2, |
| | TFEC, C1orf54, FRMD4A, CD180, LYN, CD74, IL8, AP1S2, DSE, |
| | RAB32, IL1B, RTN1, C15orf48, APLP2, SCIMP, RRAS, NAGK, IRAK3, |
| | GNS, IFIT3, TYMP, HLA-DRB1, IL13RA1, TNFSF13B, CTSH, |
| | RASSF4, CD63, LPCAT2, ITGB5, PHACTR1, NAV1, EPS8, SPRED1, |
| | FGD2, MEF2C, ACSL1, GAS7, TRIB1, LY86, IER3, FRMD4B, HLA- |
| | DQA1, KCNMB1, UACA, APOC1, ITGAX, NFIC, SEPP1, ZNF503, |
| | ST3GAL6, FN1, SORT1, MAF, RXRA, LILRB1, SLC43A2, IL1R2, |
| | TMEM51, FAM46A, PLTP, ENG, VCAN, ALCAM, METTL7A, ISG15, |
| | OGFRL1, CD276, EPB41L2, SAMHD1, CD83, CREB5, ANXA2, BTK, |
| | ADM, MYO1E, PPT1, LGALS1, IRS2, DOK3, IFI6, ALDH1A1, PRKCD, |
| | TMEM176B, SUCNR1, LRRK2, ATP6V1B2, LGALS9, LAIR1, CEBPA, |
| | LPL, HLA-DPA1, SKAP2, MTRNR2L12, IL18, SLC12A1, PLEKHO1, |
| | PAK1, PLXNB2, HLA-DPB1, FAM26F, APOE, ITPRIPL2, IFIT1, EGR2, |
| | SLAMF8, DST, HLA-DMB, GM2A, RRBP1, GOLIM4, CYSTM1, CBR1, |
| | CLEC10A, HLA-DQA2, BCL6, SLC40A1, OLR1, AXL, OTUD1, ATF3, |
| | GPR34, PIK3AP1, DOCK4, CAPG, CNTLN, TBXAS1, SPP1, IRF8, |
| | HLA-DMA, CCL3, NFKBIZ, LPAR6, GNAQ, IMPA2, POU2F2, CD9, |
| | F13A1, ATF5, NEXN, LILRA3, UMOD, YWHAH, SOD2, HSPA1B, |
| | RRAGD, PPM1N, DEPTOR, CD93, HVCN1, TNFSF10, ARHGAP18, |
| | SNX9, PHACTR2 |
| AMP Kid | EMP1, NRP1, SPP1, CD36, IL1RN, MITF, LHFPL2, CD276, EPB41L1, |
| Monocyte | SIRPA, SDC2, GPNMB, SLC1A3, TREM2, LRP1, MGLL, LPL, ITGB5, |
| Cluster 3 | PLTP, C1QC, MYO1E, BLVRB, STAB1, SPRED1, SASH1, MSR1, |
| | SLC11A1, ACTN1, APOE, YBX3, NCF2, HNMT, LMNA, SLCO2B1, |
| | CD9, C5AR1, TNS1, CSF1R, EMILIN2, MAFB, DAB2, FPR3, NAV1, |
| | TIMP2, LILRB4, ACSL1, FMNL2, ENG, CXCL16, LGMN, BCAT1, |
| | SIGLEC1, ADAP2, PTGS1, SPI1, ST14, SLC16A10, CD68, SLAMF8, |
| | EMR2, LTBR, PLAUR, KCNMB1, GSN, PLXNB2, C1QA, RBM47, |
| | TTYH3, CTSD, ADM, ITSN1, SORT1, MYOF, FCGRT, GAS7, C2, |
| | CREG1, FCGR2A, MS4A7, LGALS3, PTMS, CPA3, SLC15A3, C3AR1, |
| | FGD4, GPX1, CD163, C15orf48, CTSB, RAB31, ANPEP, ATP1B1, |
| | LGALS1, TGFBI, RNF130, CD14, TYMP, ABCA1, SGK1, CYFIP1, FTL, |
| | EPB41L3, HSPA1B, LILRB2, LIPA, ATF3, NPC2, KIAA1598, ZNF503, |
| | ALCAM, CPVL, RHOB, CTSL, TYROBP, C1QB, TUBA1C, CCDC88A, |
| | ZFHX3, GNS, PDK4, EPS8, DMXL2, PSAP, TPSAB1, CBR1, APOC1, |
| | CEBPB, KCTD12, HSPA1A, SLC31A2, CREB5, SDCBP, S100A11, |
| | IFI30, TCF7L2, SCIN, MARCKS, PILRA, A2M, BRI3, CA2, HM13, |
| | CLIC4, HK3, SERPING1, PEA15, TNFSF13B, RAB32, FRMD4A, CCL2, |
| | GRN, RGL1, DSE, HTRA1, MFSD1, GLIS3, RRAGD, ATF5, PTAFR, |
| | CPM, IFI6, GLUL, FCER1G, GOT1, SOD2, SLC8A1, HSPB1, IFNGR2, |
| | CLSPN, TNFAIP2, CD93, DOCK4, RXRA, CYSTM1, CD83, GOLIM4, |
| | SLC43A2, PLEKHO1, ITPRIPL2, IGSF6, SECTM1, PLXDC2, SCARB2, |
| | IL18, CCR1, ZNF385A, TUBA1B, SLC17A9, APLP2, NFIC, CSF2RB, |
| | TFEC, ANXA2, SDC4, FNIP2, CD63, ITGAX, ASAH1, ANXA5, CAPG, |
| | RASSF4, KCNK6, CST3, VIM, TLR2, ETS2, SLC7A7, LGALS9, AIF1, |
| | GPX3, CDKN1A, PLAU, TRIB1, LY86, FLNB, EDN1, LGALS3BP, |
| | LPCAT2, IL6R, RRBP1, SLC44A1, CEBPD, GGH, KIF2C, MNDA, |
| | CHST15, GM2A, CNTLN, ANXA4, LYZ, FADS1, TMEM51, RRAS, |
| | EFHD1, PHLDA1, PAQR5, S100A9, KIF15, LGALS2, VASH1, RGCC, |
| | ALDH1A1, AHR, VSIG4, MLEC, YWHAH, SOX4, EPB41L2, MS4A4A, |
| | AGPAT9, NAGK, OLR1, GADD45A, FSCN1, C1orf54, HCK, SUCNR1, |
| | IL13RA1, PABPC4, RNASE1, CFD, TSPAN3, IFIT3, CTNND1, RUFY3, |
| | G0S2, CXorf21, TUBB4B, FARP1, CPEB4, INSR |
| AMP Kid | AC073218.2, ACSL1, ADAMTS1, ADM, AHI1, AIF1L, ALDH1A1, |
| Kidney Tubulue | ALDH2, ALDH6A1, AMOTL2, ANK3, ANXA4, APLP2, APP, AQP2, |
| Cluster 4 | AQP3, ARHGAP24, ARSD, ASAHI, ASAP2, ATN1, ATP1A1, ATP1B1, |
| | ATP6V0A4, ATP6V1A, ATP6V1B1, ATP6V1G3, BACE2, BCAM, |
| | BCL6, BICC1, BLNK, C14orf105, C19orf77, C1orf168, C1orf54, |
| | C7orf41, CA12, CA2, CADM1, CADPS2, CALB1, CAMK2N1, CAPG, |
| | CASR, CBR1, CCND1, CD59, CD63, CD82, CD9, CDH1, CDH16, |
| | CEBPD, CFI, CGNL1, CKB, CLCN5, CLCNKA, CLCNKB, CLDN10, |
| | CLDN16, CLDN8, CLMN, CLU, CMTM4, COBLL1, COL18A1, |
| | COL4A2, COL4A3, COL6A1, CPVL, CREG1, CTB-27N1.1, CTDSPL, |
| | CTNND1, CTSD, CTSH, CTSL, CXCL14, CXCL2, CXXC5, CYFIP1, |
| | CYP1B1, CYS1, CYSTM1, DAAM1, DAB2, DCDC2, DDR1, DEFB1, |
| | DEPTOR, DMRT2, DSG2, DSP, DST, DUSP9, DYNC2H1, DZIP3, |
| | EDN1, EFHD1, ELF3, EMP1, EMX1, EMX2, ENAM, EPB41L1, |
| | EPB41L3, EPCAM, ERBB4, ESRRG, FAM134B, FAM171A1, FARP1, |
| | FCGBP, FEN1, FGD4, FGF9, FKBP2, FLNB, FNDC3B, FNIP2, FOXI1, |
| | FRMD4A, FXYD2, G0S2, GABARAPL1, GADD45A, GATA2, GLIS3, |
| | GNAQ, GNG12, GNG7, GNS, GOLIM4, GOLM1, GOT1, GP2, GPR110, |
| | GPR116, GPRC5B, GPX3, GRB14, GSN, GSTM3, GSTP1, HDLBP, |
| | HELLS, HES1, HIP1, HMGCS2, HNF1B, HNMT, HOOK1, HOXA7, |
| | HOXB6, HOXB7, HOXD10, HOXD8, HPGD, HPN, HSD11B2, |
| | HSD17B12, HSPA1A, HSPA1B, HSPB1, IDH2, IFIT3, IFITM3, IGFBP5, |
| | IGFBP7, IMPA2, INADL, IRX2, ITGA2, ITGAV, ITGB5, ITM2C, |
| | ITPRIPL2, ITSN1, JUP, KCNJ1, KCNJ10, KCNJ16, KCNQ1OT1, |
| | KIAA1522, KIF12, KIF21A, KNG1, KRT19, LAMB1, LAPTM4B, |
| | LARP1B, LGALS3, LGALS3BP, LGMN, LGR4, LHX1, LIFR, LIMA1, |
| | LINC00982, LMAN1, LMNA, LMO7, LPCAT2, LTBR, MAGI1, MAL, |
| | MAL2, MANEA, MAP9, MECOM, METTL7A, MGLL, MITF, MLLT4, |
| | MPC1, MTRNR2L8, MTUS1, MUC1, MUC15, MYO10, MYO1E, |
| | MYO6, MYOF, NAV2, NDRG2, NEDD4L, NFIB, NFIC, NGFRAP1, |
| | NPNT, NR2F2, NRP1, NTRK2, NUDT3, OBSL1, OGDHL, OSBPL10, |
| | PALLD, PAQR5, PAWR, PAX2, PAX8, PBX1, PCDH9, PCK1, PCLO, |
| | PCYOX1, PDE1A, PDE1C, PDE4D, PDK4, PEBP1, PFKFB3, PFN2, |
| | PHGDH, PIGR, PKHD1, PKP4, PLAU, PLS3, PLXNB1, PLXNB2, |
| | POU3F3, PPAPDC1B, PPARGC1A, PPP1R1A, PPP2R3A, PRDM16, |
| | PRKAA2, PRSS23, PTGER3, PTH1R, PTPN13, PTPN3, PTPRF, |
| | RAB3IP, RAP1GAP, RASD1, RBBP8, RBM47, RBPMS, RDH10, RDX, |
| | RGL3, RHBG, RHCG, RHOBTB3, RNF130, RP11-834C11.4, RRAGD, |
| | RUFY3, SASH1, SCARB2, SCD5, SCIN, SCML1, SCN2A, SCNN1A, |
| | SCNN1G, SDC1, SDC2, SDC4, SEMA6D, SEPP1, SERINC2, |
| | SERPINA5, SERPING1, SFRP1, SH3BP4, SHCBP1, SHROOM3, SIM1, |
| | SIM2, SLC12A1, SLC16A12, SLC25A4, SLC26A7, SLC3A1, SLC43A2, |
| | SLC5A3, SLC8A1, SLIT2, SMIM14, SNX9, SOD2, SORL1, SORT1, |
| | SOX4, SOX6, SPINK1, SPINT2, SPP1, SPTBN1, SRGAP1, SSPN, ST14, |
| | STRBP, SULT1C2, TACSTD2, TBC1D4, TCEA3, TCF7L2, TFAP2A, |
| | TFAP2B, TFCP2L1, THSD7A, TIMP3, TMEM176A, TMEM176B, |
| | TMEM213, TMEM51, TMEM72, TMPRSS2, TNS1, TPD52, TRIM2, |
| | TRPM3, TSPAN1, TSPAN13, TSPAN3, TSPAN6, TSPAN7, TUBA1C, |
| | UACA, UCHL1, UGT8, UMOD, USP2, USP53, VDR, VEGFA, WBP5, |
| | WFDC2, WLS, WNK4, WWC1, YBX3, YWHAH, ZFHX3, ZNF503, |
| | ZNF618, ZNF704 |
| AMP Kid | LILRB2, RP11-290F20.3, HK3, CDKN1C, SERPINA1, LST1, |
| Monocyte | APOBEC3A, AIF1, MS4A7, TCF7L2, LRRC25, SLC11A1, FCN1, |
| Cluster 5 | EMR2, MAFB, AC104809.4, CFD, HCK, LILRA3, SPI1, ANPEP, |
| | C5AR1, PILRA, CYBB, IFI30, DMXL2, SECTM1, S100A9, SLC7A7, |
| | IFITM3, S100A8, PPM1N, FCER1G, SLC2A6, RRAS, WARS, CFP, |
| | CEBPB, POU2F2, TYROBP, CD68, CST3, CTSS, HMOX1, PSAP, |
| | IRAK3, LYPD2, SIGLEC10, PLAUR, COTL1, CSF1R, LYN, KLF4, |
| | BCL2A1, SAT1, L1TD1, CD300E, FTL, SLC31A2, NFKBIZ, CLEC7A, |
| | S100A11, ADRBK2, NAMPT, CLEC12A, TIMP1, DUSP6, RXRA, |
| | EDN1, RHOB, RNF144B, PLXNB2, CEBPA, SLC8A1, TYMP, FPR1, |
| | MYOF, P2RX1, DOK3, EMILIN2, LILRB1, MARCKS, BRI3, NR4A1, |
| | NCF2, ADAP2, CKB, FGR, LMO2, RAB31, MS4A4A, MPEG1, MTSS1, |
| | FCGR3A, TLR2, FGL2, TNFAIP2, C1orf162, SOD2, ETS2, CXCL16, |
| | SCIMP, LYZ, MT2A, CUX1, IGSF6, LYST, SLC43A2, LGALS1, GNS, |
| | CCDC88A, CD86, ITGAX, FMNL2, C3AR1, PIK3AP1, VCAN, FGD2, |
| | TTYH3, CD83, CHST15, ZEB2, PAK1, SLC15A3, BTK, TNFSF13B, |
| | RGS2, IFNGR2, TLR4, SOX4, CTSL, FNIP2, LPL, NPC2, IL1RN, BCL6, |
| | LRP1, DUSP5, HES1, MARCH1, FGD4, LGALS9, CDKN1A, EPS8, |
| | PRKCD, LRRK2, GRN, ASAH1, LPCAT2, EPB41L3, TIMP2, CD14, |
| | ACPP, RNF130, FAM26F, VDR, SORT1, SAMHD1, NAGK, JUP, |
| | SPRED1, ANXA5, CHST2, ACSL1, P2RY13, LINC00936, IL1B, PDK4, |
| | TNFSF10, ATP6V1B2, ATP1B3, CTSB, RBM47, CD180, PLEKHO1, |
| | OAS1, HSPA1A, TBXAS1, CBFA2T3, KCTD12, PRR11, CTBP2, |
| | SASH1, ATF3, RBBP8, KCNQ1OT1, ITSN1, APLP2, C2, LGALS3, IFI6, |
| | SKAP2, ZFHX3, ARID3A, TGFBI, ISG15, ANXA2, SCARB2, OTUD1, |
| | CKAP4, EGR3, CYP1B1, PLXDC2, AP1S2, EFHD2, MGLL, HLA- |
| | DRB5, CSF2RB, SNX9, MNDA, SDCBP, MX2, YBX3, C1QA, CNTLN, |
| | TNF, C3, RP11-1143G9.4, TUBA1A, CX3CR1, FCGRT, INSR, PLEK, |
| | HNMT, FCGR2A, TMEM176B, PABPC4, RGL1, ABCA1, GBP2, |
| | BCAT1, LIMS1, MFSD1, PTPN13, ALOX5, OAS3, SWAP70, C1QB, |
| | LILRB4, CD36, IFIT3, TRIB1, LTBR, DUSP1, METTL7A, HSPA1B, |
| | GLUL, CD4, OGFRL1, CCR1, RASSF4, CEBPD, ATP1A1, LAIR1, IL8, |
| | ZNF385A, CD79B, CD276, CPVL, SCRN1, DPYSL2, YWHAH, RAB32, |
| | SIRPA, SGK1, GPR155, SIGLEC1, MYO1E, UBE2J1, ALDOB, CTSD, |
| | GAS7, IFIT2, EAF2, CXCL2, KLF2, HSPB1, KCNMB1, GPX3 |
| AMP Kid | AC096579.7, ADAM28, ADM, AHI1, AIM2, AL928768.3, ALCAM, |
| Plasma Cell | ALDH6A1, APOBEC3B, AQP3, ARID3A, ASF1B, ASPM, ATF5, ATN1, |
| Cluster 6 | ATP6V1C2, AURKB, BASP1, BHLHE41, BIRC5, BLNK, BRCA2, |
| | BTG2, BTK, C10orf118, C19orf10, CADM1, CADPS2, CALR, CASC5, |
| | CAV1, CBFA2T3, CBR1, CCDC112, CCDC50, CCDC88A, CCNB1, |
| | CCNB2, CCR2, CD180, CD19, CD27, CD38, CD40, CD59, CD79A, |
| | CD79B, CD9, CDC20, CDCA2, CDCA5, CDK14, CDKN1A, CDKN1C, |
| | CDKN2C, CEP128, CEP152, CHD7, CHPF, CHST15, CHST2, CITED2, |
| | CKAP4, CLIC4, CLMN, COBLL1, COL4A3, CPEB4, CPNE5, CREB3L2, |
| | CRELD2, CSF2RB, CTA-250D10.23, CTSH, CXorf21, CXXC5, |
| | CYSTM1, DAPP1, DENND5B, DERL3, DLGAP5, DNAH8, DNAJB9, |
| | DNAJC3, DOK3, DST, DUSP5, EAF2, ELL2, ENAM, ENTPD1, |
| | EPB41L1, ERLEC1, FAM171A1, FAM46C, FCGR2B, FCHSD2, FCRL2, |
| | FCRL5, FCRLA, FEN1, FKBP11, FKBP2, FNDC3B, FZD3, GADD45A, |
| | GAS6, GGH, GLCCI1, GM2A, GNG7, GOLIM4, GOT1, GPX1, GTSE1, |
| | HDAC9, HDLBP, HERPUD1, HIPK2, HJURP, HLA-DOB, HM13, |
| | HMMR, HOOK1, HOXB7, HSD17B12, HSP90B1, HSPA5, IDH2, |
| | IFNGR2, IGF1, IGHA1, IGHA2, IGHG1, IGHG2, IGHG3, IGHG4, IGJ, |
| | IGKJ3, IGKJ4, IGKJ5, IGKV1-5, IGKV3-11, IGKV3-20, IGKV4-1, |
| | IGLC2, IGLC3, IGLC7, IGLJ3, IGLL1, IGLL5, IGLV1-40, IGLV2-11, |
| | IGLV2-14, IGLV2-23, IGLV2-8, IGLV3-1, IGLV6-57, IL6R, IL6ST, |
| | INSR, IRF4, ITGA6, ITGA8, ITM2C, ITPRIPL2, KCNK6, KCNN3, |
| | KCNQ1OT1, KIAA0101, KIAA0125, KIAA1524, KIF14, KIF15, |
| | KIF21A, KIF2C, LARP1B, LAX1, LILRB4, LMAN1, LMNA, MAN1A1, |
| | MANEA, MANF, MARCKS, MBNL2, MEF2C, MEI1, METTL7A, |
| | MGLL, MLEC, MLLT4, MPC1, MTUS1, MYBL2, MY06, MZB1, |
| | NAV1, NCAPG, NCAPH, NET1, NPC2, NUCB2, NUF2, NUSAP1, |
| | OSBPL10, P2RX1, P2RX5, PABPC4, PARM1, PAWR, PCYOX1, PDIA4, |
| | PDK1, PFN2, PHGDH, PIM2, PLCG2, PLK1, PLTP, PMEPA1, PNOC, |
| | POLQ, POU2AF1, PPAPDC1B, PRC1, PRCP, PRDM1, PRDX4, PRKCD, |
| | RAB30, RALGPS2, RASD1, RASGRP3, RBBP8, RBM47, RDX, RGCC, |
| | RGS16, RNASE6, RP11-16E12.2, RP11-356I2.4, RRAGD, RRBP1, |
| | SCARB2, SCRN1, SDC1, SDF2L1, SEC11C, SEC14L1, SEL1L, SEL1L3, |
| | SHCBP1, SLAMF7, SLC17A9, SLC1A4, SLC25A4, SLC44A1, SLC7A7, |
| | SMPD3, SPAG4, SPATS2, SPINT2, SPTBN1, SRM, SSPN, SSR3, SSR4, |
| | ST14, STAP1, STRBP, SYK, TBC1D9, TCF4, TIMP2, TK1, TNFAIP2, |
| | TNFRSF13B, TNFRSF17, TOP2A, TPD52, TPX2, TRAM2, TRIB1, |
| | TROAP, TSHZ2, TSPAN1, TSPAN13, TSPAN3, TXNDC11, TXNDC15, |
| | TYMS, UAP1, UBE2C, UBE2J1, UCHL1, UMOD, VDR, VEGFA, VIMP, |
| | WARS, WBP5, XBP1, XCR1, ZBP1 |
| AMP Kid | STAB1, C1QC, FOLR2, RNASE1, SEPP1, VSIG4, CD14, SIGLEC1, |
| Monocyte | CD163, F13A1, MS4A6A, C1QA, SLCO2B1, MSR1, ADAP2, C1QB, |
| Cluster 7 | PLTP, SLC40A1, SLC1A3, IGF1, APOE, NRP1, FPR3, FCGRT, CSF1R, |
| | MAFB, MS4A4A, TGFBI, CPVL, MARCKS, LGMN, TNFAIP2, MS4A7, |
| | TLR4, GPR34, DAB2, ITSN1, PLXDC2, SERPING1, AXL, APOC1, |
| | CYBB, CSF3R, TIMP2, C2, CD68, KCTD12, TREM2, A2M, AIF1, |
| | CCL8, TMEM176B, FSCN1, SIRPA, GRN, CST3, LRP1, CXCL12, |
| | ZFHX3, TLR2, CXCL16, FCGR2A, ETV5, TMEM51, RNASE6, |
| | EPB41L3, FRMD4A, HNMT, HLA-DRB1, FMNL2, SRGAP1, CTSB, |
| | CEBPD, MPEG1, CSF2RA, CD74, DOCK4, KIAA1598, HLA-DRA, |
| | GSN, NPC2, PTAFR, CREG1, FRMD4B, PDK4, CLEC7A, BLVRB, |
| | PSAP, RASSF4, TYROBP, CD302, CYFIP1, IL8, IER3, GAS6, EPS8, |
| | DMXL2, LTBR, CD276, SECTM1, RNF130, LRRC25, HMOX1, ITGB5, |
| | ABCA1, RTN1, CCDC88A, FTL, C3, C5AR1, LYZ, ADORA3, CD93, |
| | SLC15A3, LILRB2, SPRED1, CD86, GPR82, LHFPL2, DUSP6, |
| | LPCAT2, TTYH3, CPM, LILRB4, HLA-DPA1, SASH1, MTUS1, RGL1, |
| | P2RY13, TMEM176A, MEF2C, MARCH1, IGSF6, ETS2, ITPRIPL2, |
| | IFI30, IFITM3, GPX1, TFEC, HCK, CXCL3, PILRA, LGALS3BP, KLF4, |
| | C3AR1, HLA-DRB5, C1orf54, HLA-DPB1, SLC7A7, RBM47, RAB31, |
| | CXCL2, IL18, SERPINA1, LY86, MNDA, HLA-DMB, CTSL, CREB5, |
| | PTGS1, ST3GAL6, HLA-DQA1, NCF2, DSE, IL13RA1, ZNF385A, |
| | FGD2, CCR1, FAM26F, CNTLN, CCL3, FCER1G, CD4, ENG, IFNGR2, |
| | YBX3, CLEC10A, RAB32, LAIR1, LST1, SPI1, CFD, BCAT1, |
| | SLC43A2, CTSH, EPB41L2, SAT1, CCL2, TNFSF13B, MFSD1, SDCBP, |
| | PLD4, OGFRL1, SLC8A1, GLUL, ZNF618, CTSS, HLA-DQB1, |
| | PLEKHO1, FGL2, SCARB2, TLR7, CTNND1, EMR2, CADM1, |
| | ATP1B1, GPNMB, ALDH1A1, NAGK, HLA-DMA, NFIC, FGD4, SYK, |
| | LPAR6, SLC31A2, PLAUR, SLAMF8, WLS, DST, MAF, CEBPB, TNS1, |
| | SGK1, RP11-1143G9.4, TBXAS1, IFNGR1, MYO1E, DOK3, BRI3, |
| | ADRBK2, HSPA1A, CEBPA, LIPA, ZNF503, ALDH2, MEF2A, ASAH1, |
| | FCGBP, CD36, TYMP, LGALS2, IRAK3, LMO2, RHOB, EGR1, CIITA, |
| | ALCAM, GAS7, ARHGAP18, FILIP1L, PEA15, C1orf162, SNX9, |
| | ENTPD1, JUP, BMP2K, MITF, GNS, PPT1, APLP2, TSPAN33, IFI27, |
| | GOLIM4, ANKRD22, TCF4, PRKCD, PAK1, MGLL, PTMS, OLR1, |
| | LINC00936, UACA, CCND1, CXCL10, EMILIN2, LYN, PLXNB2, IL1B, |
| | HSPA1B, DPYSL2, ADAM28, VASH1, SPP1, ALOX5, HLA-DOA, |
| | NAV1, TBC1D9, ZEB2, PLAU, FPR1, SORT1, CD83, IFI6, SPECC1, |
| | IL6R, KCNMB1, SERPINF1, EGR3, LGALS9, TUBA1B, ATF3, ITGAX, |
| | RNF144B, FCN1, CCDC112, CD300E, PHACTR1, CD63, BLNK, |
| | LMNA, KCNK6, RUFY3, AP1S2, YWHAH, SWAP70, ATP6V1B2, |
| | HDAC9, ARHGAP24, HSPB1, ANXA5, GM2A, NAMPT, FAM46A, |
| | SKAP2, BAZ2B, ACTN1, NFIA, PKIB, LILRB1, VCAN, CLCN5, |
| | BRCA2, TRIB1, PALD1, CTSD, METTL7A, GPX3, CD9, ITGAV, LPL, |
| | TCF7L2, HLA-DQB2, IMPA2, GPR155, RNU1-60P, RHOBTB3, MX2, |
| | IRS2, CXorf21, SAMHD1, MYOF, HVCN1, AMICA1, AHR, S100A11, |
| | TIMP1, CSF2RB, PTPRE, SCML1, RXRA, SCIMP, SCRN1, CLEC9A, |
| | MTSS1, FNIP2, RRAS, FARP1, S100A9, MYO10, NUDT3, ACSL1, |
| | CLEC12A, DEPTOR, PRCP, STMN1, ST14, CTDSPL, CFP, OTUD1, |
| | ATF5, HLA-DQA2, EPB41L1, PIK3AP1, INSR, SEC14L1, RRAGD, |
| | EGR2, GAPT, WDFY4, IL1RN, GNAQ |
| AMP Kid | A2M, ABCA1, ACPP, ACSL1, ACTN1, ADAM28, ADAP2, ADORA3, |
| Dendritic | ADRBK2, AFF3, AGPAT9, AHI1, AHR, AIF1, AKR1C3, ALCAM, |
| Cluster 8 | ALDH1A1, ALDH2, ALDOB, ALOX5, ALOX5AP, AMICA1, |
| | ANKRD22, ANKRD26, ANXA5, AP1S2, APLP2, APOC1, APOE, APP, |
| | ARHGAP18, ARHGAP24, ARHGAP5, ARSD, ASAH1, ATF3, ATF5, |
| | ATP1B1, ATP6V1B2, AXL, BASP1, BAZ2B, BCAT1, BCL6, BHLHE41, |
| | BLNK, BLVRB, BMP2K, BRCA2, BRI3, BRI3BP, BTK, C15orf48, |
| | C1orf162, C1orf54, C1QA, C1QB, C1QC, C2, C3, C3AR1, C5AR1, |
| | C7orf41, CADM1, CAPG, CCDC112, CCDC50, CCDC88A, CCL2, |
| | CCL3, CCL8, CCND1, CCR1, CCR2, CD14, CD163, CD1C, CD1E, |
| | CD276, CD302, CD4, CD68, CD74, CD83, CD84, CD86, CD9, CD93, |
| | CDKN1A, CDKN1C, CEBPA, CEBPB, CEBPD, CFD, CHD7, CHST15, |
| | CIITA, CKB, CLCN5, CLDN1, CLEC10A, CLEC12A, CLEC5A, |
| | CLEC7A, CLEC9A, CLIC4, CNTLN, CPM, CPVL, CREB5, CREG1, |
| | CSF1R, CSF2RA, CSF2RB, CSF3R, CST3, CTBP2, CTNND1, CTSB, |
| | CTSH, CTSL, CUX1, CXCL12, CXCL16, CXorf21, CYBB, CYFIP1, |
| | DAAM1, DAB2, DAPP1, DEPTOR, DOCK4, DPYSL2, DSE, DSG2, |
| | DST, DUSP6, EGR1, EGR2, EGR3, ELL2, EMILIN2, EMR2, ENG, |
| | ENTPD1, EPB41L2, EPB41L3, EPS8, ETS2, ETV5, F13A1, FADS1, |
| | FAM129A, FAM26F, FAM46A, FARP1, FCER1A, FCER1G, FCGBP, |
| | FCGR2A, FCGR2B, FCGRT, FCHSD2, FGD2, FGD4, FGL2, FILIP1L, |
| | FKBP5, FLT3, FMNL2, FN1, FNIP2, FPR1, FPR3, FRMD4A, FRMD4B, |
| | FSCN1, FTL, GAPT, GAS6, GAS7, GLUL, GM2A, GNAQ, GNS, |
| | GOLIM4, GPNMB, GPR155, GPR183, GPR34, GPR82, GPX1, GPX3, |
| | GRN, GSN, GSTM3, GSTP1, HCK, HDAC9, HERPUD1, HLA-DMA, |
| | HLA-DMB, HLA-DOA, HLA-DPA1, HLA-DPB1, HLA-DQA1, HLA- |
| | DQA2, HLA-DQB1, HLA-DQB2, HLA-DRA, HLA-DRB1, HLA-DRB5, |
| | HMOX1, HNMT, HSD17B12, HSPA1A, HSPA1B, HTRA1, HVCN1, |
| | IDO1, IER3, IFI30, IFNGR1, IFNGR2, IGF1, IGHG4, IGSF6, IL13RA1, |
| | IL18, IL1B, IL1R2, IL6R, IL8, IMPA2, INSR, IRAK3, IRF8, IRS2, |
| | ITGAM, ITGAV, ITGAX, ITGB5, ITPRIPL2, ITSN1, KCNK6, |
| | KCNMB1, KCTD12, KIAA1598, KLF4, LAIR1, LGALS2, LGALS3BP, |
| | LHFPL2, LILRB2, LILRB4, LIMS1, LINC00936, LMO2, LMO7, LPAR6, |
| | LPCAT2, LPL, LRP1, LRRC25, LRRCC1, LRRK2, LST1, LTBR, LY86, |
| | LYN, LYZ, MAFB, MAN1A1, MARCH1, MARCKS, MBNL2, MEF2A, |
| | MEF2C, METTL7A, MFSD1, MIR24-2, MIS18BP1, MITF, MLLT4, |
| | MNDA, MPEG1, MS4A4A, MS4A6A, MS4A7, MSR1, MTRNR2L8, |
| | MYADM, MYO1E, MYOF, NAGK, NAMPT, NAV1, NCF2, NCKAP5, |
| | NDRG2, NEDD4L, NET1, NFIA, NFIC, NPC2, NR4A1, NR4A2, NR4A3, |
| | NRP1, NUDT3, OGFRL1, OLR1, OTUD1, P2RX1, P2RY13, PABPC4, |
| | PAK1, PALD1, PALLD, PCK1, PEA15, PFKFB3, PHACTR1, PIGR, |
| | PILRA, PKIB, PLAU, PLAUR, PLD4, PLEKHO1, PLTP, PLXDC2, |
| | PLXNB2, PMAIP1, PMEPA1, PPT1, PRCP, PRKCD, PRR11, PSAP, |
| | PTAFR, PTGS1, PTMS, PTPRE, RAB31, RAB32, RASGRP3, RASSF4, |
| | RBM47, RBPJ, RDX, RGL1, RGS1, RGS10, RGS16, RGS2, RHOB, |
| | RHOBTB3, RNASE6, RNF130, RNF144B, RNU1-60P, RP11-1143G9.4, |
| | RP11-693J15.5, RP11-834C11.4, RPS4Y1, RTN1, RUFY3, S100A11, |
| | S100B, SAT1, SCARB2, SCIMP, SCIN, SCML1, SCRN1, SDC2, SDCBP, |
| | SEC14L1, SEMA6D, SEPP1, SERPINA1, SERPINF1, SERPING1, |
| | SESN3, SGK1, SH3BP4, SHCBP1, SIGLEC1, SIGLEC10, SIRPA, |
| | SKAP2, SKIL, SLAMF8, SLC11A1, SLC12A3, SLC15A3, SLC17A9, |
| | SLC1A3, SLC1A4, SLC31A2, SLC40A1, SLC43A2, SLC44A1, SLC7A7, |
| | SLC8A1, SLCO2B1, SMIM14, SORL1, SORT1, SOX4, SPATS2, |
| | SPECC1, SPI1, SPINK1, SPINT2, SPP1, SPRED1, SRGAP1, SSPN, |
| | SSR3, STI4, ST3GAL6, STAB1, STX7, SUCNR1, SWAP70, SYK, |
| | TBC1D9, TBXAS1, TCF4, TFCP2L1, TFEC, TGFBI, TIAM1, TIMP2, |
| | TLR10, TLR2, TLR4, TLR7, TMCC3, TMEM176A, TMEM176B, |
| | TMEM51, TMEM52B, TNFAIP2, TNFSF13B, TREM2, TRIB1, TSPAN3, |
| | TSPAN33, TTYH3, TUBA1A, TUBA1B, TYMP, TYROBP, UACA, |
| | USP53, VASH1, VSIG4, WDFY4, WLS, YBX3, YWHAH, ZBTB16, |
| | ZFHX3, ZMAT1, ZNF385A, ZNF503, ZNF618 |
| AMP Kid | CLEC10A, RP11-1143G9.4, FLT3, LGALS2, CD1E, CD1C, CSF2RA, |
| Dendritic | FCER1A, LYZ, IL1R2, CPVL, ZNF385A, RTN1, RAB32, PKIB, IDO1, |
| Cluster 9 | ALDH2, KIAA1598, IL13RA1, CST3, CD93, NAV1, BASP1, KCTD12, |
| | SPECC1, TGFBI, MNDA, NDRG2, SLAMF8, DSE, CD302, CIITA, |
| | AFF3, C1orf54, RAB31, IL18, HLA-DQB1, HLA-DRB1, SERPINF1, |
| | SLC8A1, HLA-DRA, IGSF6, HLA-DQA1, IL8, CBFA2T3, HLA-DMB, |
| | PHACTR1, PAK1, HLA-DPB1, HLA-DPA1, GSN, CCDC88A, HLA- |
| | DOA, PLD4, FAM26F, AGPAT9, MS4A6A, AMICA1, FGL2, SH3BP4, |
| | FCGRT, KCNMB1, ST3GAL6, CLEC12A, KCNK6, KLF4, LTBR, IL1B, |
| | HLA-DMA, SPI1, GRN, EPB41L2, MPEG1, CSF3R, TSPAN33, CD86, |
| | ENTPD1, ITPRIPL2, XCR1, HDAC9, LY86, HCK, BCL11A, CLEC9A, |
| | ADAP2, FPR3, CLEC7A, DPYSL2, AXL, AHR, AIF1, CD74, CREB5, |
| | CFP, NET1, WDFY4, PRCP, GPX1, RNASE6, MARCH1, ACPP, CTSH, |
| | TNFAIP2, TNFSF13B, FGD2, FNIP2, F13A1, LST1, PLXNB2, |
| | ADAM28, FILIP1L, CD163, CD36, OGFRL1, HLA-DQA2, VSIG4, HLA- |
| | DRB5, RUFY3, NCF2, PEA15, ETS2, LRRK2, P2RY13, HLA-DQB2, |
| | RASSF4, ADRBK2, PTAFR, FSCN1, C1orf162, NPC2, SRGAP1, CSF1R, |
| | ADORA3, MEF2C, ITGAX, TIMP2, PLXDC2, RBM47, VASH1, |
| | CCDC112, ALCAM, MYO1E, GNAQ, C15orf48, LPCAT2, NCKAP5, |
| | TBC1D9, SYK, SIRPA, AP1S2, TLR2, LILRB4, MFSD1, GSTP1, DST, |
| | ANPEP, TLR10, CXCL16, LMO2, MYOF, LRRC25, RNF130, CYFIP1, |
| | EMILIN2, IL6R, ACTN1, SOX4, YBX3, SPINT2, IMPA2, ST14, |
| | IFNGR1, DAPP1, TFEC, CSF2RB, CNTLN, HIP1, SLC31A2, ALOX5, |
| | VCAN, PPT1, GAPT, CXCL9, BCL6, IRAK3, SECTM1, CEBPA, |
| | PLEKHO1, SAMHD1, ZFHX3, CAPG, CXorf21, SCRN1, SIGLEC1, |
| | LRP1, TACSTD2, PTGS1, CEBPD, PLAUR, PARM1, GAS6, CLCN5, |
| | FCGR2A, BTK, IFI30, BRI3BP, SCIMP, LMNA, HNMT, FGD4, GAS7, |
| | CD68, TLR7, CCR2, NAGK, CYBB, ZNF503, SLC15A3, GM2A, |
| | CTNND1, GOLIM4, COTL1, FCGR2B, DEPTOR, PRKCD, YWHAH, |
| | BLVRB, IFNGR2, INSR, RGS10, TBXAS1, CD4, PTPRE, FMNL2, |
| | LGMN, ATP1B1, BRI3, SLC43A2, C19orf10, PABPC4, CD83, S100B, |
| | RBBP8, PMAIP1, PALD1, FAM46A, SIGLEC10, TCF4, EPB41L3, |
| | SLC2A6, FRMD4B, HLA-DOB, CCND1, KIT, EMR2, CREG1, |
| | TMEM176B, GCSAM, LILRA4, GPR183, ATF5, NAMPT, PILRA, |
| | SERPINA1, EMP1, SGK1, PRR11, MARCKS, SRM, RGS2, TUBA1C, |
| | TYROBP, SPATS2, SKAP2, TMCC3, PRC1, ETV5, MIS18BP1, |
| | LGALS9, IRF8, SHCBP1, BMP2K, CLNK, CD 180, MEF2A, APP, |
| | PALLD, ATP6V1B2, SLC1A3, RNF144B, RNU1-60P, FAM111B, |
| | TYMP, TIMP1, FADS1, IGFBP7, KIAA1524, LGALS3, ARHGAP5, |
| | CTBP2, LINC00936, METTL7A, FCER1G, LYN, DAB2, TMEM51, |
| | MLEC, CPM, EGR1, SMIM14, STX7, CCDC18, ENG, HSD17B12, |
| | RGL1, CTSB, TUBA1A, OLR1, CD14, CUX1, FAM129A, BCAT1, IER3, |
| | FZD3, TMEM176A, CCR1, STAB1, APLP2, TSPAN3, IRF4, RBPJ, TK1, |
| | FCN1, WARS, NR4A3, KIAA0101, LRRCC1, ATP6V1A, PLAU, |
| | ZNF618, ATF3, BRCA1, FCHSD2, ASAP2, PSAP, MTRNR2L8, SORT1, |
| | MSR1, LGALS1, CADM1, FPR1, CD300E, LILRB2, ANXA2, CD59, |
| | MYADM, CEP128, PHACTR2, EPS8, A2M, TUBA1B, LARP1B, SPIB, |
| | TCEA3, CCDC50, SPRED1, UBE2C, VIM, ARSD, CEP55, MS4A7, |
| | BIRC5, IFITM3, CDH1, ANXA5, TTYH3, SEC14L1, EGR3, MS4A4A, |
| | AURKB, TLR4, CLSPN, C1QB, PDIA4, CCR5, KIAA0226L, CTSS, |
| | SLC12A3, SLC17A9, CDCA2, IDH2, CENPF, TROAP, PKP4, STMN1, |
| | JUP, RRBP1, SLC40A1, GPX3, BAZ2B, CASC5, SSR3, OTUD1, NRP1, |
| | POLQ, PRDX4, RGS1, PLK1, SERPING1, SWAP70, MIR24-2, ITGAV, |
| | VDR, SLC7A7, CLEC5A, PTGER3, KIF2C, ACSL1, CLMN, NFKBIZ, |
| | HVCN1 |
| AMP Kid B | ADAM28, ADRBK2, AFF3, AIM2, ALOX5, ARHGAP24, BACE2, |
| Cells Cluster 10 | BANK1, BASP1, BCL11A, BLNK, BMP2K, BRCA2, BTK, CACNA1A, |
| | CAPG, CBFA2T3, CCDC50, CCR7, CD180, CD19, CD22, CD38, CD40, |
| | CD69, CD74, CD79A, CD79B, CD83, CD86, CDK14, CHD7, CIITA, |
| | CMPK2, COBLL1, COCH, CPNE5, CTA-250D10.23, CTSH, CXCL10, |
| | CXCR5, CXorf21, CYBB, DAPP1, DDX58, DDX60, DENND5B, |
| | DNAH8, DSP, DUSP5, EAF2, EBF1, ENTPD1, FAIM3, FAM129C, |
| | FAM46A, FAM65B, FCGR2B, FCHSD2, FCRL1, FCRL2, FCRL5, |
| | FCRLA, FGD2, FLNB, GAPT, GBP1, GBP4, GM2A, GNG7, GSN, |
| | HERC5, HLA-DMA, HLA-DMB, HLA-DOA, HLA-DOB, HLA-DPA1, |
| | HLA-DPB1, HLA-DQA1, HLA-DQB1, HLA-DQB2, HLA-DRA, HLA- |
| | DRB1, HVCN1, ID3, IDO1, IFI44L, IFI6, IFIH1, IFIT1, IFIT2, IFIT3, |
| | IGFBP4, IGHD, IGHM, IRF4, IRF8, ISG15, JUP, KIAA0040, KIAA0125, |
| | KIAA0226L, LGALS9, LILRB1, LINC00926, LMO2, LTB, LY86, LYN, |
| | MARCH1, MARCKS, MEF2C, MNDA, MPEG1, MS4A1, MT2A, MX1, |
| | MX2, MYBL2, NEXN, OAS1, OAS3, OASL, OSBPL10, P2RX5, PAWR, |
| | PAX5, PIK3AP1, PLAC8, PLCG2, PLEKHO1, PMAIP1, PMEPA1, |
| | PNOC, POU2AF1, POU2F2, RAB30, RALGPS2, RASGRP3, RP11- |
| | 693J15.5, RSAD2, SCIMP, SELL, SKAP2, SMC6, SMIM14, SPIB, SSPN, |
| | STAP1, STAT1, STX7, SWAP70, SYK, TBC1D9, TCF4, TCL1A, TFEC, |
| | TLR10, TLR7, TMCC3, TNFRSF13B, TNFSF10, TNFSF13B, TRIB1, |
| | TSPAN13, TSPAN33, TTN, UGT8, VPREB3, WARS, WDFY4, ZBP1 |
| AMP Kid | AFF3, AHI1, ALCAM, ALDH2, ALOX5AP, AMICA1, AP1S2, APP, |
| Mixed Myeloid | ARID3A, BAZ2B, BCL11A, BLNK, BRCA2, BRI3BP, C10orf118, |
| B Cluster 11 | C12orf75, CAPG, CBFA2T3, CCDC18, CCDC50, CCDC88A, CCR2, |
| | CD36, CD4, CD68, CD74, CDH1, CEP128, CIITA, CLCN5, CLMN, |
| | COBLL1, CREB3L2, CSF2RA, CSF2RB, CST3, CTSB, CUX1, CXorf21, |
| | CXXC5, CYBB, DAAM1, DAB2, DENND5B, DERL3, DMXL2, |
| | DNAJB9, DNAJC3, DPYSL2, DST, DUSP5, ENAM, FAM129C, |
| | FCER1A, FCER1G, FCHSD2, FCRLA, FGD2, FKBP2, FLNB, FLT3, |
| | FZD3, GAPT, GAS6, GPR183, GPX1, GRN, GSN, GZMB, HDAC9, |
| | HERPUD1, HIP1, HLA-DMB, HLA-DOA, HLA-DQA1, HLA-DRA, |
| | HSP90B1, HVCN1, IGHM, IGJ, IGKJ5, IGLV2-8, IGSF6, INSR, IRF4, |
| | IRF8, ITM2C, KCTD12, KIAA0226L, KIAA1598, LAIR1, LARP1B, |
| | LGMN, LHFPL2, LILRA4, LILRB4, LYN, MANF, MARCH1, MEF2A, |
| | MEF2C, MLEC, MPEG1, MS4A6A, MYBL2, MYO1E, MZB1, NAGK, |
| | NFKBIZ, NPC2, NRP1, NUCB2, P2RX1, PDIA4, PHACTR1, PLAC8, |
| | PLD4, PLEK, PLXNB2, PMEPA1, PRKCD, PSAP, PTGDS, PTPRE, |
| | RASD1, RGS1, RNASE6, RNF144B, RNU1-60P, RP11-356I2.4, RRBP1, |
| | SAMHD1, SCARB2, SCRN1, SEL1L3, SELL, SERPINF1, SKAP2, |
| | SLC12A3, SLC2A6, SLC40A1, SMC6, SMIM14, SMPD3, SNX9, SOX4, |
| | SPATS2, SPIB, SPINT2, SSR3, ST14, STMN1, STRBP, STX7, SYK, |
| | TCF4, TCL1A, TFEC, TGFBI, TLR7, TNFSF13B, TSPAN13, TSPAN3, |
| | UBE2J1, VASH1, VIMP, WDFY4, ZNF618 |
| AMP Kid | CD74, CD79A, GZMK, HLA-DQA1, HLA-DRA, MTRNR2L2, TRBC2 |
| Unknown | |
| Cluster 12 | |
| AMP Kid B | BANK1, CD22, CD79A, MS4A1, PAX5, FCRLA, CD19, LINC00926, |
| cells Cluster 13 | CTA-250D10.23, FCRL2, RALGPS2, SPIB, FCRL1, TLR10, FCRL5, |
| | VPREB3, EBF1, FAM129C, RP11-693J15.5, BCL11A, CPNE5, TCF4, |
| | POU2AF1, KIAA0226L, CD79B, MARCH1, BLNK, CD74, CD180, |
| | OSBPL10, ADAM28, SWAP70, HVCN1, WDFY4, HLA-DOB, AIM2, |
| | IGHD, HLA-DRA, HLA-DQB1, SCIMP, CD40, GAPT, HLA-DQA1, |
| | P2RX5, HLA-DMB, ARHGAP24, AFF3, IGHM, TNFRSF13B, IRF8, |
| | CIITA, HLA-DRB1, HLA-DPB1, HLA-DOA, SMIM14, RASGRP3, |
| | HLA-DMA, FCGR2B, MEF2C, DENND5B, CXCR5, STX7, KIAA0125, |
| | GNG7, CDK14, FAIM3, HLA-DPA1, SYK, BASP1, STAP1, SSPN, |
| | STRBP, POU2F2, DAPP1, TCL1A, ID3, TSPAN3, DOK3, CHD7, PNOC, |
| | TSPAN13, LY86, BHLHE41, PAWR, COCH, CXXC5, COBLL1, |
| | CCDC50, BACE2, AL928768.3, CBFA2T3, BTK, ALOX5, CD82, |
| | FCHSD2, TLR7, TFEC, TTN, SEL1L3, LRMP, PLCG2, COL4A3, EAF2, |
| | TBC1D9, FGD2, RAB30, BIRC3, IGHA1, SCRN1, ADRBK2, LTB, |
| | SELL, CACNA1A, PCDH9, CCDC141, BMP2K, HLA-DQB2, SMC6, |
| | METTL7A, CYBB, LRRK2, TSPAN33, TPD52, IGHG1, HDAC9, |
| | ARID5B, IGKJ5, CAPG, UBE2J1, GM2A, DNAH8, FCRL3, IFNGR2, |
| | AC096579.7, SIGLEC10, LYN, DAAM1, PHACTR1, PLEKHO1, HLA- |
| | DRB5, BTG2, SESN3, UGT8, HLA-DQA2, CCR7, CTSH, FAM65B, |
| | CD200, GPR183, PARM1, IGHG3, ZMAT1, ENTPD1, MEF2A, SKAP2, |
| | CD83, IRF4, MPEG1, KIAA0040, PIK3AP1, IGLV3-21, MTSS1, |
| | MARCKS, CD1C, IFI30, ANXA4, IGLC2, SPTBN1, BRI3BP, CLIC4, |
| | MZB1, IGKV4-1, IGLC3, ARID3A, CXorf21, EGR1, SPINT2, CDKN1A, |
| | PLAC8, PPAPDC1B, IGHA2, PMEPA1, RAB31, CD86, CXCR4, YBX3, |
| | PRKCD, OTUD1, KLF2, IGHG2, ZNF480, ALCAM, HERPUD1, |
| | SLC17A9, RNASE6, CLMN, TMCC3, MNDA, LILRB1, TNFRSF17, |
| | IFI44L, DERL3, LL22NC03-2H8.5, CLNK, CHST15, ZNF827, SKIL, |
| | IGHG4, HCK, IGKJ4, IGJ, MX1, JUP, PLD4, IGKV1-5, CD27, SLC2A6 |
| AMP Kid CD4 | ALOX5AP, ANK3, ARHGAP5, ARL4C, BCL11B, BIRC3, BRCA2, |
| T cell Cluster | CCR7, CD2, CD27, CD28, CD3D, CD3E, CD3G, CD4, CD40LG, CD5, |
| 14 | CD84, CD96, CMPK2, CTLA4, CXCL10, CXCL13, DAPP1, DDX58, |
| | DDX60, DGKH, FAM134B, FOXP3, GBP1, GBP2, GBP4, GBP5, |
| | GIMAP5, GIMAP7, GPR155, GPR171, GZMK, HERC5, ICOS, IFI44L, |
| | IFI6, IFIH1, IFIT1, IFIT2, IFIT3, IGFBP4, IL32, IL6R, IL6ST, IL7R, |
| | INPP4B, ISG15, ITGA6, ITM2A, KIAA0040, LEF1, LGALS3BP, |
| | LGALS9, LIMS1, LTB, MAF, MAL, MT2A, MX1, MX2, NEXN, OAS1, |
| | OAS3, OASL, PDCD1, PEBP1, PIM2, PTPN13, RBPJ, RGS1, RORA, |
| | RP11-94L15.2, RSAD2, RTKN2, SAMHD1, SAT1, SELL, SIRPG, |
| | SOCS3, SPOCK2, ST8SIA1, STAT1, SYNE2, TBC1D4, TCF7, THEMIS, |
| | TIAM1, TIGIT, TNFRSF25, TNFSF10, TRABD2A, TRAC, TRAT1, |
| | TRBC2, TSHZ2, ZBP1 |
| AMP Kid CD4 | AHI1, AHR, ARID5B, BAZ2B, BCL11B, BIRC3, C10orf118, CADM1, |
| T Cell Cluster | CAV1, CCDC141, CCDC50, CCR7, CD2, CD200, CD247, CD27, CD28, |
| 15 | CD3D, CD3E, CD3G, CD4, CD40LG, CD5, CD59, CD7, CD82, CD84, |
| | CD96, CDK14, CLNK, CPM, CREB3L2, CTLA4, CTSB, CXCL13, |
| | CXCR5, DDX60, DGKH, DUSP2, DUSP4, EGR2, EGR3, ENTPD1, |
| | FAIM3, FAM46C, FCRL3, FGFR1, FKBP5, FOXP3, FRMD4B, FZD3, |
| | GADD45A, GAPDH, GATA3, GBP2, GBP5, GLCCI1, GNG4, GOLGB1, |
| | GPR155, HDAC9, HELLS, ICOS, ID3, IGFBP4, IGFL2, IKZF2, IKZF4, |
| | IL2RB, IL32, IL6R, IL6ST, IL7R, INPP4B, IRF4, ISG15, ITM2A, |
| | KLRB1, LAX1, LEF1, LIMA1, LIMS1, LMAN1, LTB, MAF, MAL, |
| | MAST4, MX1, NELL2, NFIA, NMB, PDCD1, PDE3B, PDE4D, PEBP1, |
| | PHACTR2, PHLDA1, PIM2, PLK1, PLS3, POU2AF1, PPAPDC1B, |
| | PRKCH, PTPN13, PTTG1, PYHIN1, RBPJ, RDH10, RGCC, RGS1, |
| | RGS10, RORA, RTKN2, SEC11C, SELL, SERPINE2, SESN3, SH2D1A, |
| | SIRPG, SMC4, SNX9, SPOCK2, ST8SIA1, STAT1, SYNE2, TBC1D4, |
| | TCF7, TIAM1, TIGIT, TNFRSF25, TOP2A, TOX2, TRAC, TRAT1, |
| | TRBC2, TRIB1, TSHZ2, TXNDC11, VDR |
| AMP Kid CD4 | AQP3, ARID5B, ARL4C, BCL11B, BIRC3, CAMK4, CCR7, CD2, |
| T Cell Cluster | CD247, CD27, CD28, CD3D, CD3E, CD3G, CD4, CD40LG, CD5, CD82, |
| 16 | CD84, CD96, CISH, CTLA4, CXCR4, CXCR5, DGKH, FAIM3, |
| | FAM134B, FCRL3, FGFR1, FKBP5, FOXP3, GAPDH, GATA3, GBP2, |
| | GBP5, GIMAP5, GIMAP7, GLCCI1, GPR155, HSPB1, ICOS, ID3, |
| | IGFL2, IKZF2, IKZF4, IL2RB, IL32, IL6R, IL6ST, IL7R, INADL, |
| | INPP4B, ITGA6, ITM2A, LEF1, LIMA1, LPAR6, LTB, MAL, MAST4, |
| | NGFRAP1, PDE3B, PDK1, PEBP1, PHACTR2, PIM2, PRKCH, RGCC, |
| | RGS10, RORA, RTKN2, SELL, SESN3, SH2D1A, SIRPG, SOCS3, |
| | SORL1, SPOCK2, SPTBN1, ST8SIA1, STAT1, SYNE2, TBC1D4, TCF7, |
| | TIAM1, TIGIT, TNFRSF25, TOX2, TRABD2A, TRAC, TRAT1, TRBC2, |
| | TSHZ2, TTN, TXK, VIM |
| AMP Kid CD4 | ALOX5AP, AMICA1, AQP3, ARL4C, BCL11B, CAMK4, CCL5, CCR5, |
| CD8 T Cell | CD2, CD27, CD28, CD3D, CD3E, CD3G, CD4, CD40LG, CD5, CD69, |
| Cluster 17 | CD84, CD8A, CD8B, CD96, CTLA4, CXCL13, CXCR3, CXCR4, |
| | CXCR6, DGKH, DUSP4, DZIP3, FKBP5, GAPDH, GATA3, GBP5, |
| | GIMAP5, GIMAP7, GLCCI1, GZMA, GZMK, ICOS, IGHA1, IGHA2, |
| | IGHG1, IGHG2, IGHG3, IGJ, IGLC3, IGLV2-8, IL32, IL6ST, IL7R, |
| | INADL, INPP4B, ITGA1, ITM2A, ITM2C, JUN, LEF1, LTB, MAF, |
| | MIAT, OASL, PDCD1, PEBP1, PTTG1, RAB3IP, RBPJ, RORA, RP11- |
| | 94L15.2, SEL1L3, SH2D1A, SIRPG, SPOCK2, ST8SIA1, SYNE2, TCF7, |
| | THEMIS, TIGIT, TNFSF14, TOX2, TRAC, TRAT1, TRBC2, TTN, VIM, |
| | XCL1, ZNF683 |
| AMP Kid | ABCA1, AC073218.2, ACPP, ACSL1, ADM, AGPAT9, AHI1, AIF1L, |
| Kidney Tublule | ALCAM, ALDH1A1, ALDH2, ALDH6A1, AMOTL2, ANK3, |
| Cluster 18 | ANKRD22, ANKRD26, ANXA4, APLP2, APOC1, APOE, APP, AQP2, |
| | ARHGAP18, ARHGAP24, ARHGAP5, ARID3A, ARSD, ASAH1, |
| | ASAP2, ATF3, ATN1, ATP1A1, ATP1B1, ATP1B3, ATP6V0A4, |
| | ATP6V0D2, ATP6V1A, ATP6V1B1, ATP6V1C2, ATP6V1G3, AUTS2, |
| | BACE2, BAZ2B, BCAM, BICC1, BLNK, BLVRB, BRI3, BRI3BP, |
| | C10orf118, C14orf105, C19orf77, C1orfl68, C1orf54, C7orf41, CA12, |
| | CA2, CADM1, CADPS2, CALB1, CAMK2N1, CAPG, CASR, CBR1, |
| | CCDC34, CCDC50, CCDC80, CCND1, CD276, CD59, CD63, CD9, |
| | CDH1, CDH16, CDK14, CDKN1C, CEBPD, CEP290, CFI, CGNL1, |
| | CHD7, CHPF, CISH, CKAP4, CKB, CLCN5, CLCNKA, CLCNKB, |
| | CLDN10, CLDN16, CLDN8, CLMN, CLNK, CLU, CMTM4, CNTLN, |
| | COBLL1, COL18A1, COL1A2, COL4A2, COL4A3, COL6A1, CPA3, |
| | CPEB4, CPM, CPVL, CREB3L2, CREG1, CRELD2, CST3, CTB-27N1.1, |
| | CTBP2, CTDSPL, CTNND1, CTSB, CTSD, CTSH, CTSL, CUX1, |
| | CXCL12, CXCL14, CXXC5, CYFIP1, CYP1B1, CYS1, CYSTM1, |
| | DAAM1, DAB2, DCDC2, DDR1, DEFB1, DENND5B, DEPTOR, |
| | DMRT2, DMXL2, DNAJC3, DOCK4, DSG2, DSP, DST, DUSP9, |
| | DYNC2H1, DZIP3, EFHD1, EGF, ELF3, EMP1, EMX1, EMX2, ENAM, |
| | EPB41L1, EPB41L3, EPCAM, EPS8, ERBB4, ERLEC1, ESRRG, ETV5, |
| | FAM134B, FAM171A1, FARP1, FCGBP, FCGRT, FEN1, FGD4, FGF9, |
| | FGFR1, FKBP2, FLNB, FMNL2, FNDC3B, FNIP2, FOXI1, FRMD4A, |
| | FRMD4B, FXYD2, FZD3, G0S2, GABARAPL1, GADD45A, GAS6, |
| | GATA2, GGH, GLIS3, GLUL, GNAQ, GNG12, GNG7, GNS, GOLGB1, |
| | GOLIM4, GOLM1, GOT1, GP2, GPNMB, GPR110, GPR116, GPR56, |
| | GPRC5B, GPX3, GRB14, GSN, GSTM3, GSTP1, HDLBP, HELLS, |
| | HES1, HIP1, HIPK2, HMGCS2, HNF1B, HNMT, HOOK1, HOXA7, |
| | HOXB6, HOXB7, HOXD10, HOXD8, HPGD, HPN, HSD11B2, |
| | HSD17B12, HSPA1A, HSPA1B, HSPB1, ID3, IDH2, IER3, IFITM3, |
| | IGFBP5, IGFBP7, IKZF4, IL13RA1, IL18, IL1R2, IL6ST, IMPA2, |
| | INADL, INSR, IRS2, IRX2, ITGA1, ITGA2, ITGA6, ITGAV, ITGB5, |
| | ITM2C, ITPRIPL2, ITSN1, IVNS1ABP, JUP, KCNJ1, KCNJ10, KCNJ16, |
| | KCNN3, KCNQ1OT1, KCTD12, KIAA1522, KIAA1598, KIF12, |
| | KIF21A, KIF23, KIT, KNG1, KRT19, LAMB1, LAPTM4B, LARP1B, |
| | LGALS3, LGALS3BP, LGMN, LGR4, LHX1, LIFR, LIMA1, |
| | LINC00982, LL22NC03-2H8.5, LMAN1, LMNA, LMO7, LPCAT2, LPL, |
| | LRRCC1, LRRK2, LTBR, MAGI1, MAL, MAL2, MAN1A1, MANEA, |
| | MANF, MAP9, MAST4, MBNL2, MECOM, MEF2A, METTL7A, |
| | MFSD1, MFSD4, MGLL, MITF, MLEC, MLLT4, MPC1, MT1G, |
| | MTRNR2L12, MTRNR2L8, MTSS1, MTUS1, MUC1, MUC15, MYO10, |
| | MYO1E, MYO6, MYOF, NAV2, NCKAP5, NDRG2, NEDD4L, NET1, |
| | NEXN, NFIA, NFIB, NFIC, NGFRAP1, NPC2, NPNT, NR2F2, NRP1, |
| | NTRK2, NUCB2, NUDT3, OBSL1, OGDHL, OSBPL10, OTUD1, |
| | PALLD, PAQR5, PAWR, PAX2, PAX8, PBX1, PCDH9, PCK1, PCLO, |
| | PCYOX1, PDE1A, PDE1C, PDE4D, PDIA4, PDK4, PEA15, PEBP1, |
| | PFN2, PHACTR1, PHGDH, PIGR, PKHD1, PKP4, PLAU, PLS3, |
| | PLXNB1, PLXNB2, PMEPA1, POU3F3, PPAPDC1B, PPARGC1A, |
| | PPP1R1A, PPP2R3A, PPT1, PRCP, PRDM16, PRKAA2, PRKCD, |
| | PRSS23, PTGER3, PTH1R, PTMS, PTPN13, PTPN3, PTPRF, RAB3IP, |
| | RALGPS2, RAP1GAP, RASD1, RBBP8, RBM47, RBPMS, RCAN2, |
| | RDH10, RDX, RGL3, RHBG, RHCG, RHOB, RHOBTB3, RNF130, |
| | RNF144B, RNF165, RP11-834C11.4, RPL39, RRAGD, RRBP1, RUFY3, |
| | RXRA, SASH1, SCARB2, SCD5, SCIN, SCML1, SCN2A, SCNN1A, |
| | SCNN1G, SCRN1, SDC1, SDC2, SDC4, SEC14L1, SEMA6D, SEPP1, |
| | SERINC2, SERPINA5, SERPING1, SFRP1, SH3BP4, SHROOM3, SIM1, |
| | SIM2, SLC12A1, SLC12A3, SLC16A10, SLC16A12, SLC25A4, |
| | SLC26A7, SLC3A1, SLC40A1, SLC43A2, SLC44A1, SLC5A3, SLIT2, |
| | SMC2, SMIM14, SNX9, SOD2, SORL1, SORT1, SOX4, SOX6, SPATS2, |
| | SPINK1, SPINT2, SPP1, SPRED1, SPTBN1, SRGAP1, SSR3, ST14, |
| | ST3GAL6, STMN1, STRBP, SUCNR1, SULT1C2, SYTL2, TACSTD2, |
| | TBC1D4, TBC1D9, TCEA3, TCF7L2, TFAP2A, TFAP2B, TFCP2L1, |
| | THSD7A, TIMP3, TMEM176A, TMEM176B, TMEM213, TMEM51, |
| | TMEM52B, TMEM72, TMPRSS2, TNS1, TPD52, TPSAB1, TRIB1, |
| | TRIM2, TRPM3, TSPAN1, TSPAN3, TSPAN33, TSPAN6, TSPAN7, |
| | TUBA1C, TUBB4B, TXNDC15, UACA, UAP1, UCHL1, UGT8, UMOD, |
| | USP2, USP53, VDR, VEGFA, VIMP, WBP5, WFDC2, WLS, WNK4, |
| | WWC1, YBX3, ZFHX3, ZMAT1, ZNF480, ZNF503, ZNF618, ZNF704, |
| | ZNF827 |
| AMP Kid CD4 | ALOX5AP, AMICA1, ANK3, ANXA1, AQP3, ARHGAP5, ARL4C, |
| T Cell Cluster | AUTS2, BCL11B, BIRC3, BTG2, CAMK4, CCR2, CCR7, CD2, CD28, |
| 19 | CD3D, CD3E, CD3G, CD4, CD40LG, CD5, CD69, CD82, CD84, CD96, |
| | CHD7, CISH, CITED2, CXCR3, CXCR4, CXCR6, DDIT4, DGKH, |
| | DUSP1, DUSP2, EGR1, FAM129A, FAM134B, FKBP11, FKBP5, FOS, |
| | FOSB, GATA3, GCSAM, GIMAP5, GIMAP7, GNAQ, GPR155, GPR171, |
| | GPR183, GZMK, HMGB2, HOOK1, HPGD, HSPB1, ICOS, ID2, IL32, |
| | IL6R, IL6ST, IL7R, INADL, INPP4B, ITGA1, ITGA6, ITM2A, JUN, KIT, |
| | KLRB1, LEF1, LTB, MAF, MAL, MAST4, MYADM, NELL2, PDE3B, |
| | PDE4D, PEBP1, PFKFB3, PIM2, PRKCH, PTGER2, PTPN13, RAB3IP, |
| | RBPJ, RGCC, RGS10, RGS16, RORA, RP11-35612.4, RP11-94L15.2, |
| | RPL39, SIRPG, SLC16A10, SOCS3, SORL1, SPOCK2, ST8SIA1, |
| | SYNE2, SYTL2, TBC1D4, TCEA3, TCF7, THEMIS, TNF, TNFAIP3, |
| | TNFRSF25, TNFSF14, TRABD2A, TRAC, TRAT1, TRBC2, TXK, |
| | USP53, VIM, XCL1, ZBTB16 |
| AMP Kid | ADM, ALDOB, AMOTL2, APLP2, APOBEC3A, ATP1B1, ATP6V0A4, |
| Kidney Cluster | ATP6V0D2, ATP6V1B1, ATP6V1G3, C14orf105, C1orf168, C2, CA12, |
| 20 | CA2, CALB1, CAV1, CCDC80, CCL2, CCL8, CD163, CDH1, CFI, |
| | CLCNKA, CLCNKB, CLDN8, CLU, COL14A1, COL1A2, COL3A1, |
| | COL6A1, CTB-27N1.1, CXCL12, CXCL14, CXCR4, CXCR6, CYP1B1, |
| | CYS1, DEFB1, DMRT2, DSG2, EBF1, EFHD1, EMX1, EMX2, EPCAM, |
| | ERBB4, ESRRG, FN1, FOXI1, FXYD2, GATA2, GNG12, GOLM1, |
| | GPNMB, GPR110, GPR116, GPRC5B, GRB14, HMGCS2, HPN, |
| | HSD11B2, IFI30, IGFBP5, IGFBP7, IL7R, KIF12, KIT, LIFR, LMO7, |
| | MAGI1, MAL2, MTRNR2L12, MTRNR2L8, MUC1, NFIC, NGFRAP1, |
| | OGDHL, PCK1, PDE1A, PDE1C, PLS3, PPARGC1A, PRG4, PTGER3, |
| | PTPN3, RAB3IP, RAP1GAP, RASD1, RBPMS, RHBG, RHCG, RP11- |
| | 356I2.4, RPL39, SCIN, SCN2A, SCNN1A, SDC2, SDC4, SEMA6D, |
| | SFRP1, SHROOM3, SIM1, SLC16A10, SLC26A7, SLIT2, SOD2, |
| | SPINK1, SULT1C2, TFCP2L1, THSD7A, TIMP3, TMEM213, |
| | TMEM52B, TMPRSS2, TNS1, TSHZ2, TSPAN6, TSPAN7, UAP1, |
| | VEGFA, WBP5 |
| AMP Kid | A2M-AS1, ADAMTS1, AKR1C3, ANXA1, ARL4C, AUTS2, C12orf75, |
| NK/NKT Cell | C1orf21, CCL3, CCL4, CCL5, CD160, CD247, CD300A, CD38, CD63, |
| Cluster 21 | CD7, CEBPD, CEP290, CEP78, CHST2, CMC1, CST7, CTBP2, CTSD, |
| | CTSW, CX3CR1, CXCR2, CXXC5, DNAJC3, DTHD1, EFHD2, EOMES, |
| | FAM65B, FASLG, FCER1G, FCGR3A, FCRL3, FCRL6, FGFBP2, FGR, |
| | FNDC3B, GABARAPL1, GADD45B, GAS7, GATA3, GBP4, GBP5, |
| | GFOD1, GIMAP5, GIMAP7, GLCCI1, GNLY, GNPTAB, GOLIM4, |
| | GOLM1, GPR56, GSTP1, GZMA, GZMB, GZMH, HIPK2, HOPX, |
| | HPGD, HSP90B1, HSPA5, ID2, IFNG, IGFBP7, IGLV1-40, IKZF2, |
| | IL18RAP, IL2RB, IL32, ITGAM, ITGAX, IVNS1ABP, JAKMIP2, |
| | KIR2DL2, KIR3DL1, KLF2, KLRB1, KLRC1, KLRC2, KLRD1, KLRF1, |
| | KLRG1, LAG3, LAIR1, LAIR2, LDLR, LGALS1, LITAF, LYN, LYST, |
| | MAN1A1, MTSS1, MYBL1, MYO6, MYOM2, NCAM1, NKG7, NR4A2, |
| | NUCB2, OASL, PALLD, PATL2, PDE4D, PHLDA1, PLAC8, PLEK, |
| | PRDM1, PRF1, PRKCH, PRR5L, PRSS23, PTGDR, PTGDS, PTGER2, |
| | PTPN12, PTPRE, PYHIN1, RHOBTB3, RNF165, RORA, RP11-94L15.2, |
| | RUNX3, S1PR5, SAMD3, SAMHD1, SBK1, SH2D1A, SH2D1B, |
| | SLAMF7, SLC5A3, SORL1, SPON2, SYNE2, SYTL2, TBX21, TGFBR3, |
| | TKTL1, TNFSF14, TRDC, TRGC1, TRGC2, TTC38, TXK, TYROBP, |
| | UAP1, XBP1, XCL2, ZBTB16, ZEB2, ZNF683 |
| AMP Kid NK | ALDOB, AMOTL2, APLP2, ATF3, C1orf21, C2, CA12, CALB1, CAV1, |
| Cell Cluster 22 | CCDC80, CCL2, CNTLN, COL14A1, COL1A2, COL3A1, COL6A1, |
| | CXCL12, CXCR2, CYP1B1, EBF1, EFHD1, EPB41L2, EPB41L3, |
| | FGFBP2, FGL2, FN1, GGH, GNLY, GPNMB, GPR116, GZMB, IFI30, |
| | IGFBP5, IGFBP7, IL18RAP, ITGA6, KLRB1, KLRC1, KLRC2, KLRF1, |
| | KRT19, MTRNR2L12, MTRNR2L8, MYOM2, NCAM1, NFIA, NFIB, |
| | NGFRAP1, PRG4, PRSS23, S1PR5, SEMA6D, SFRP1, SH2D1B, |
| | SLC12A1, SMIM14, SOCS3, SOD2, SPON2, TIMP3, TMEM176A, TXK, |
| | UACA, USP2 |
| AMP Kid | ADAMTS1, AKR1C3, ALOX5AP, ANXA1, ANXA2, ANXA4, APLP2, |
| NK/NKT Cell | ARL4C, ARSD, ASF1B, ASPM, ATAD5, ATP1B3, AURKB, AUTS2, |
| Cluster | BIRC5, BRCA1, C12orf75, C1orf21, CALR, CCL3, CCL4, CCL5, |
| | CCNB1, CCNB2, CD160, CD247, CD300A, CD38, CD63, CD69, CD7, |
| | CDC20, CDCA2, CDCA5, CDK1, CDK6, CDKN2C, CEBPD, CENPE, |
| | CENPF, CEP55, CEP78, CHST2, CISH, CKAP2L, CLSPN, CMC1, CST7, |
| | CTBP2, CTSD, CTSW, CX3CR1, CXCR2, CXXC5, DAB2, DDIT4, |
| | DLGAP5, DTHD1, DUSP6, EFHD2, EOMES, FADS1, FAM46A, |
| | FAM65B, FANCI, FASLG, FCER1G, FCGR3A, FCRL3, FCRL6, FEN1, |
| | FGFBP2, FGR, FKBP11, FNDC3B, GADD45B, GAS7, GATA3, GBP1, |
| | GBP2, GBP4, GBP5, GFOD1, GIMAP5, GIMAP7, GLUL, GNLY, |
| | GNPTAB, GOLM1, GPR171, GPR56, GSTP1, GTSE1, GZMA, GZMB, |
| | GZMH, HIPK2, HJURP, HMMR, HOPX, HPGD, HSD17B12, HSP90B1, |
| | HSPA5, ID2, IFITM3, IFNG, IGFBP7, IKZF2, IL18RAP, IL2RB, ITGAD, |
| | ITGAM, ITGAX, IVNS1ABP, JAKMIP2, KIAA0101, KIF14, KIF15, |
| | KIF21A, KIF23, KIR2DL2, KIR3DL1, KLF2, KLRB1, KLRC1, KLRC2, |
| | KLRD1, KLRF1, LAG3, LAIR1, LAIR2, LAX1, LDLR, LGALS1, |
| | LITAF, LYN, LYST, MAF, MAN1A1, MANF, MEI1, MIR24-2, MKI67, |
| | MTSS1, MYBL1, MYO6, MYOM2, NCAM1, NCAPG, NCAPH, NDC80, |
| | NKG7, NR4A2, NR4A3, NUCB2, NUF2, NUSAP1, PALLD, PATL2, |
| | PDE4D, PDIA4, PHLDA1, PIK3AP1, PLAC8, PLCG2, PLEK, PLK1, |
| | POLQ, PRC1, PRDM1, PRF1, PRKCH, PRR5L, PRSS23, PTGDR, |
| | PTGDS, PTGER2, PTPN12, PTPRE, PYHIN1, RASSF4, RHOBTB3, |
| | RNF165, RORA, RP11-94L15.2, RRBP1, RRM2, RUNX3, S100B, |
| | S1PR5, SAMD3, SAMHD1, SBK1, SCD5, SDCBP, SDF2L1, SGOL2, |
| | SH2D1B, SHCBP1, SLAMF7, SLC1A4, SLC5A3, SMC4, SORL1, |
| | SPINK1, SPON2, STMN1, SYK, SYNE2, SYTL2, TBX21, TBXAS1, |
| | TGFBR3, TKTL1, TMCC3, TOP2A, TPX2, TRDC, TRGC1, TRGC2, |
| | TROAP, TTC38, TUBB4B, TXK, TYMS, TYROBP, UAP1, UBE2C, |
| | VEGFA, XBP1, XCL2, ZBP1, ZBTB16, ZEB2 |
| AMP Kid CD8 | A2M-AS1, ALOX5AP, AMICA1, ARHGAP18, ARL4C, BCL11B, |
| T Cell Cluster | C12orf75, CCDC141, CCL4, CCL5, CCR1, CCR5, CD160, CD2, CD247, |
| 24 | CD27, CD3D, CD3E, CD3G, CD63, CD69, CD7, CD84, CD8A, CD8B, |
| | CD96, CDK6, CLNK, CMC1, COTL1, CRTAM, CST7, CTSW, CXCR3, |
| | CXCR6, DTHD1, DUSP2, DUSP4, DZIP3, EOMES, FAM129A, FASLG, |
| | FCRL6, GABARAPL1, GAPDH, GATA3, GBP5, GIMAP5, GIMAP7, |
| | GLCCI1, GPR171, GZMA, GZMH, GZMK, HIP1, HOPX, ID2, IDH2, |
| | IFNG, IL2RB, IL32, INPP4B, ITGA1, ITGAD, ITM2A, ITM2C, |
| | JAKMIP2, KIAA0040, KIF21A, KLRC1, KLRC2, KLRD1, KLRG1, |
| | KRT86, LAG3, MIAT, NDC80, NKG7, NUCB2, OASL, PATL2, PDCD1, |
| | PRF1, PRKCH, PTGDR, PTTG1, PYHIN1, RGS1, RP11-94L15.2, |
| | RPS4Y1, RUNX3, SAMD3, SEL1L3, SH2D1A, SIRPG, SLAMF7, SMC4, |
| | SMPD3, SPOCK2, ST8SIA1, STRBP, SYNE2, SYTL2, THEMIS, TIGIT, |
| | TNFSF14, TOX2, TRAC, TRAT1, TRBC2, TRDC, TRGC2, TTN, VIM, |
| | XCL1, XCL2, ZNF683, ZNF827 |
| AMP Kid CD8 | A2M-AS1, ANXA1, ARL4C, ATP1A1, ATP1B3, BCL11B, C12orf75, |
| T Cell Cluster | C1orf21, CALR, CCL4, CCL5, CD160, CD2, CD247, CD300A, CD38, |
| 25 | CD3D, CD3E, CD3G, CD5, CD69, CD7, CD8A, CD8B, CD96, CEP78, |
| | CISH, CMC1, CRTAM, CST7, CTSW, CX3CR1, DTHD1, DUSP2, |
| | EFHD2, EOMES, FAM129A, FAM65B, FASLG, FCGR3A, FCRL6, |
| | FGFBP2, FGR, FKBP11, FNDC3B, GADD45B, GAPDH, GATA3, GBP1, |
| | GBP2, GBP4, GBP5, GIMAP5, GIMAP7, GNLY, GNPTAB, GPR171, |
| | GPR56, GSTP1, GZMA, GZMB, GZMH, HOPX, HSP90B1, HSPA5, ID2, |
| | IDH2, IFNG, IL2RB, IL32, ITGAD, ITGAM, IVNS1ABP, JAKMIP2, |
| | KIF21A, KIR3DL1, KLF2, KLRD1, KLRG1, LAG3, LAIR2, LAX1, |
| | LDLR, LGALS1, LIMA1, LITAF, LYST, MIAT, MYADM, MYBL1, |
| | MYO6, NKG7, NR4A2, OAS1, OASL, PATL2, PDCD1, PLAC8, PLEK, |
| | PRDM1, PRF1, PRKCH, PRR5L, PRSS23, PTGDR, PTGDS, PTGER2, |
| | PTMS, PTPN12, PTPRE, PYHIN1, RORA, RP11-94L15.2, RSAD2, |
| | RUNX3, S1PR5, SAMD3, SAMHD1, SBK1, SH2D1A, SLAMF7, SMC4, |
| | SPON2, STAT1, STMN1, SYNE2, SYTL2, TBX21, TGFBR3, THEMIS, |
| | TIGIT, TNF, TNFSF14, TRAC, TRBC2, TRDC, TRGC1, TRGC2, |
| | TTC38, TUBB4B, XBP1, XCL2, ZEB2, ZNF683 |

Table 71H provides a list of machine learning (ML)-generated clusters, and sets of genes associated with disease activity in SLE, which were identified using methods and systems of the present disclosure as being strongly correlative with each given ML-generated cluster. These sets of genes can be used as effective SLE biomarkers to indicate disease activity via the given ML-generated clusters.

TABLE 71H
ML Generated

| ML Cluster | Genes |
|---|---|
| ML Module | RAB4B, ADAR, MRPL44, CDCA5, SNN, BRD3, |
| | C7ORF43, CDC20, SP1, POFUT1, SAMD4B, ATP6V1B2, |
| | TSPAN9, SP140, STK26, IRF4, LCP1, LMO2, SF3B4, |
| | HIST2H2AA3, CITED4, ADAM8, TICAM1, HSD17B7 |
| ML UP | CDCA5, MRPL44, SNN, C7orf43, CDC20, POFUT1, |
| | SAM44B, SP140, ADAR, LCP1, IRF5 |

Table 72A provides a list of DxterityProject clusters, and sets of genes associated with disease activity in SLE, which were identified using methods and systems of the present disclosure as being strongly correlative with each given DxterityProject cluster. These sets of genes can be used as effective SLE biomarkers to indicate disease activity via the given DxterityProject clusters.

TABLE 72A
DxterityProject_GSE45291_ZeroSL

| | |
|---|---|
| ZeroSledai | TMPO, APOL6, MDM4, N4BP2L2, SPTLC2, ARHGEF7, |
| Genes GR1 | TFDP2, TCEA1, VHL, MYL12A, NXPE3, FBXO6, |
| | TRMT2B, ZNF496, MB21D1, ZNF254, JAK2, |
| | PLEKHA2, ZNF595, PSIP1, ITGB5, FBXO9, |
| | TREML4, NAIP, ST3GAL3 |
| ZeroSledai | TMPO, APOL6, MDM4, N4BP2L2, SPTLC2, ARHGEF7, |
| Genes GR2 | TFDP2, TCEA1, VHL, MYL12A, NXPE3, FBXO6 |
| ZeroSledai | TMPO, APOL6, MDM4, N4BP2L2, SPTLC2, ARHGEF7, |
| Genes GR3 | TFDP2 |
| ZeroSledai | TMPO, APOL6, MDM4, N4BP2L2 |
| Genes GR4 | |

Table 72B provides a list of DxterityProject clusters, and sets of genes associated with disease activity in SLE, which were identified using methods and systems of the present disclosure as being strongly correlative with each given DxterityProject cluster. These sets of genes can be used as effective SLE biomarkers to indicate disease activity via the given DxterityProject clusters.

TABLE 72B
DxterityModules_24April2018

| | |
|---|---|
| IGS | IFI27, RSAD2, IFI6, MX1, HERC5, |
| | EPSTI1, SPATS2L, EIF2AK2 |
| Ikaros Genes | IKZF1, IKZF3 |
| Plasmablasts | CD38, XBP1, TNFRSF17, IGKC |
| T cells | TCF7, CD3D, CD4, CD8B, CCR7 |
| B Cells | BACH2, CD79B, FCRL1, CD27, CD19 |
| Dendritic | CLEC10A |
| LDGs | ARG1, OLFM4, ELANE |
| Energy | SLC2A3 |
| Translation | RPL27 |
| Inflammatory | IL1A, IL1B, IL1RN, TNF, CSF1, |
| screen | CSF2, CSF3, IL37 |

Table 72C provides a list of DxterityProject clusters, and sets of genes associated with disease activity in SLE, which were identified using methods and systems of the present disclosure as being strongly correlative with each given DxterityProject cluster. These sets of genes can be used as effective SLE biomarkers to indicate disease activity via the given DxterityProject clusters.

TABLE 72C
DxterityModules_10April2018

| | |
|---|---|
| IGS | IFI27, RSAD2, IFI6, MX1, HERC5, |
| | EPSTI1, SPATS2L, EIF2AK2 |
| Ikaros Genes | IKZF1, IKZF2, IKZF3 |
| Plasmablasts | CD38, XBP1, TNFRSF17, IGKC |
| T cells | TCF7, CD3D, CD4, CD8B, CCR7 |
| B Cells | BACH2, CD79B, FCRL1, CD27, CD19 |
| Monos | CD14, CD163 |
| Dendritic | CLEC10A |
| LDGs | ARG1, OLFM4, ELANE |
| Energy | SLC2A3 |
| Translation | RPL27 |

Table 73A provides a list of I-Scope gene clusters, and sets of genes associated with disease activity in SLE, which were identified using methods and systems of the present disclosure as being strongly correlative with each given I-Scope cluster. These sets of genes can be used as effective SLE biomarkers to indicate disease activity via the given I-Scope clusters.

TABLE 73A
Reilly_MissingIscopeGenes_notinpaper_2Aug2019 |
Iscope up HDAC6

| | |
|---|---|
| T & B Cells | Rag1, Msh5, Fcmr |
| NK/NKT cells | Klra7, Klrb1f, Klrc2/Klrc3, Klre1, Klra1, Klra4, |
| | Klra8, Klrb1, Klrb1b, Nkg7, Cd96, Klrd1, Klrc1, |
| | Gzma, Ctsw, Klrk1 |
| T cells | Trav3-3, Trav7-4, Trbc2, Trbv1, Trbv13-1, |
| | Trbv13-3, Trbv14, Tcrg-C1, Trac, Trav7-1, Trbc1, |
| | Trbv12-2, Trbv29, Lef1, Aire, Hcst, Ier3, Cd160, |
| | Sit1, Adora2a |
| Tact | Cxcr6, Satb1, Cd40lg, Ebi3, Nkg7, Cd96 |
| CD8 T cells | Nkg7, Cd96, Cd160, Klrd1, Klrc1, Gzma, Cd8a, |
| | Ctsw, Klrk1, Cd8b1 |
| gd T cells | Tcrg-C1, Tcrg-C2 |
| LDG | Osm, Elane, Mpo, Ctsg, Prtn3, Ms4a3, Olfm4 |
| Neutrophil | Adgrg3 |
| Bact | Cd40lg |
| T, B, Mono | AI467606 |
| Mono and B | Ebi3 |
| Ag Presentation = | Clec4a2, Tnfsf4 |
| MHCII presentation | |

Table 73B provides a list of I-Scope gene clusters, and sets of genes associated with disease activity in SLE, which were identified using methods and systems of the present disclosure as being strongly correlative with each given I-Scope cluster. These sets of genes can be used as effective SLE biomarkers to indicate disease activity via the given I-Scope clusters.

TABLE 73B
Reilly_MissingIscopeGenes_notinpaper_2Aug2019 |
Iscope down HDAC6

| | |
|---|---|
| T cells | Nlrc3, Tiam1, Shcbp1, Lat2, Ceacam1, Cd244, |
| | Cd300a, Cnr2, Il21, Pvrig, Tnfsf11, Hells |
| Treg | Uhrf1, Foxp3, Ikzf2, Tigit, Ido1 |
| Tact | Tigit, Zc3h12d, Lag3, Tnfrsf4, Cd83, Slamf1, |
| | Pdcd1, Havcr1, Ctla4, Icos, Kcna3, Tnfrsf8 |
| Tanergic | Lag3 |
| NK/NKT | Tigit, Lag3, Tnfrsf8, Cd244, Il21, Gzmk, Lpxn, |
| | Klra2, S1pr5, Lair1, Il21r, Slamf6, Mybl1 |
| T & B | Lpxn, Il21r, Slamf6, Batf, Nfam1, Malt1, |
| | Gimap4, Sash3, Vav1, Rftn1, Myo1g, Sell, Ada, |
| | Spib |
| T & B & Mono | Lair1, Ikzf3, Slamf7 |
| Mono and B | Pik3ap1, Themis2, Hck, Cd180, Cd38 |
| Ag Presenting = | Scimp, Ciita, Rfx5 |
| MHC II Presentation | |
| Neutrophil | Cd83, Lilra5, C6, Samsn1, Sell, Ceacam1, |
| | Cd300a, Fut4, Fpr1, Il17ra, C3, Siglece, Sirpb1c |
| LDG | |
| Bact | ARID3A, CD83, Pdcd1 |

### Example 38: Analysis of Gene Expression from Systemic Lupus Erythematosus Synovium Revealed a Profile of Activated Immune Cells and Inflammatory Pathway

Arthritis is a common manifestation of systemic lupus erythematosus (SLE), and the success of a new lupus therapy may depend on its ability to suppress joint inflammation. Despite this, an understanding of the underlying pathogenic mechanisms driving lupus synovitis may remain incomplete. Using systems and methods of the present disclosure, gene expression profiles of SLE synovium were interrogated to gain insight into the nature of joint inflammation in lupus arthritis.

Biopsied knee synovia from SLE and OA patients were analyzed for differentially expressed genes (DEGs) to determine similarities and differences between gene profiles and also by Weighted Gene Co-expression Network Analysis (WGCNA) to identify modules of highly co-expressed genes that correlated with clinical features of lupus arthritis. DEGs and correlated WGCNA modules were interrogated for statistical enrichment by Gene Set Variation Analysis (GSVA). Genes were functionally characterized using BIG-C and canonical pathways and upstream regulators operative in lupus synovitis were predicted by IPA. Biological upstream regulators and drug compounds targeting lupus synovitis were additionally predicted by the Library of Integrated Network-Based Cellular Signatures (LINCS).

DEGs upregulated in lupus arthritis revealed enrichment of numerous immune and inflammatory cell types dominated by a myeloid phenotype, whereas downregulated genes were characteristic of fibroblasts. WGCNA revealed seven modules of co-expressed genes significantly correlated to lupus arthritis or disease activity (SLEDAI or anti-dsDNA titer). Functional characterization of both DEGs and WGCNA modules by BIG-C revealed consistent co-expression of immune signaling molecules and immune cell surface markers, pattern recognition receptors (PRRs), antigen presentation, and interferon stimulated genes. Although DEGs were predominantly enriched in myeloid cell transcripts, WGCNA also revealed enrichment of activated T cells, B cells, CD8 T and NK cells, and plasma cells/plasmablasts indicating an adaptive immune response in lupus arthritis. Th1, Th2, and Th17 cells were not identified by transcriptomic analysis although IPA predicted signaling by the Th1 pathway and numerous innate immune signaling pathways were verified by GSVA. IPA additionally predicted inflammatory cytokines TNF, CD40L, IFNα, IFNβ, IFNγ, IL27, IL1, IL12, and IL15 as active upstream regulators of the lupus arthritis gene expression profile in addition to the PRR-related genes IRF7, IRF3, TLR7, TICAM1, IRF4, IRF5, TLR9, TLR4, and TLR3. Analysis of chemokine receptor-ligand pairs, adhesion molecules, germinal center (GC) markers and T follicular helper (Tfh) cell markers indicated trafficking of immune cell populations into the synovium by chemokine signaling, but not in situ generation of fully-formed GCs. GSVA confirmed activation of both myeloid and lymphoid cell types and inflammatory signaling pathways in lupus arthritis, whereas OA was characterized by tissue repair/damage. Numerous therapies were predicted to target the lupus synovitis gene signature including anti-TNF biologics, NFκB pathway inhibitors, MAPK inhibitors, and CDK inhibitors.

Detailed gene expression analysis was performed to identify a unique pattern of cellular components and physiologic pathways operative in lupus synovitis, as well as a host of drugs potentially able to target this common manifestation of lupus.

Systemic lupus erythematosus (SLE) may be a complex autoimmune disease in which loss of self-tolerance gives rise to pathogenic autoantibodies causing widespread inflammation and tissue damage. Whereas SLE may be characterized by multiorgan involvement and a large degree of patient heterogeneity, arthritis may be a common manifestation with 65 to 95% of lupus patients reporting joint involvement during the progression of their disease.

Despite the high frequency of lupus arthritis, an understanding of the underlying pathogenic mechanisms driving lupus synovitis may remain incomplete. Indeed, much of the information on the nature and classification of lupus arthritis may be based on clinical observation and medical imaging modalities to inform the state of joint involvement and also laboratory markers, such as an elevation in the proinflammatory cytokine IL-6 in the serum and the presence of elevated anti-double-stranded DNA (anti-dsDNA) autoantibody titers. Other autoantibodies, including anti-ribonucleoprotein (anti-RNP), anti-histone, and anti-proliferating cell nuclear antigen (anti-PCNA) may be implicated in lupus arthritis along with evidence of inflammation manifested by increased C-reactive protein (CRP) and erythrocyte sedimentation rate (ESR), although longitudinal studies may need to be performed to confirm these associations.

A lack of a better understanding of the nature of lupus arthritis may relate to the difficulty of obtaining tissue samples and the absence of relevant and reliable animal models. Despite this, in many recent clinical trials of potential lupus therapies, arthritis may be a principal manifestation, and the success of a tested therapy can depend of its ability to suppress joint inflammation. Therefore, there is a need to understand more about the pathogenic mechanisms operative in this lupus manifestation in order to evaluate the impact of potential new therapies.

Inasmuch as there is a demonstrated need for more effective therapies in lupus and a striking lack of a complete understanding of the cellular and molecular underpinnings of lupus arthritis, a more thorough understanding of molecular mechanisms underlying lupus arthritis may be informative. Global gene expression profiles and histology of SLE, RA, and osteoarthritis (OA) synovium may be analyzed to begin to elucidate the inflammatory mechanisms in each disease. Using systems and methods of the present disclosure, bioinformatic techniques were applied to assess the only lupus synovitis gene expression data set available to gain additional insight into the pathogenesis of lupus arthritis. Using a multipronged, bioinformatic and systems biology approach, a model of SLE synovitis is determined that may serve as the basis to identify new targeted therapies.

Gene expression data sourcing and processing were performed as follows. Publicly available microarray data from synovial biopsies from the knees of 4 SLE and 5 OA subjects were obtained from NCBI Gene Expression Omnibus (GEO) under accession GSE36700. Data processing and analysis were conducted within the R statistical programming platform using relevant Bioconductor packages. All raw data files underwent background correction and GCRMA normalization resulting in log 2 intensity values compiled into expression set objects (e-sets). Outliers were identified through the inspection of first, second, and third principal components and through inspection of array dendrograms calculated using Euclidean distances and clustered using average/UPGMA agglomeration. GSM899013_OA5 was consistently identified as an outlier and excluded from further analyses. Post-analysis additions to the metadata revealed this OA patient was male, whereas the SLE and OA patients included in the study were female. Low intensity probes were removed by visual assignment of a 2.34 threshold cutoff upon a histogram of binned log 2-transformed probe intensity values.

Differential gene expression analysis was performed as follows. Identification of DEGs was conducted using the LIMMA package in R. To increase the probability of finding DEGs, both Affy chip definition files (CDFs) and BrainArray (BA) CDFs were used to create and annotate e-sets, analyzed separately, then results merged. Linear models of normalized gene expression values were created through empirical Bayesian fitting. Resultant p-values were adjusted for multiple hypothesis testing using the Benjamini-Hochberg correction. Significant probes were filtered to retain a pre-specified False Discovery Rate (FDR)<0.2 and duplicate probes were removed again to retain the most significant probe. The FDR was assigned a priori to avoid excluding false negative probes.

Weighted Gene Co-expression Network Analysis was performed as follows. The same normalized and filtered data (Affy CDFs only) were processed using WGCNA to conduct an unsupervised clustering analysis, yielding statistically co-expressed modules of genes used for further biological interrogation. Per the WGCNA algorithm, a scale-free topology matrix (TOM) was calculated in each analysis to encode the network strength between probes. TOM distances were used to cluster probes into WGCNA modules. Resulting co-expression networks were trimmed to further isolate individual modules of probes using dynamic tree cutting and the deepSplit function in R. Partitioning around medoids (PAM) was also utilized to assign outliers to the nearest cluster. Modules were given random color assignments and expression profiles summarized by a module eigengene (ME). Final membership of probes representing the same gene were decided based on strongest within-module correlation to the ME value. For each module, ME values were correlated by Pearson correlation to clinical data including cohort, SLE disease activity index (SLEDAI), anti-dsDNA, C3, C4, and CRP. Cohort was represented as a binary variable where SLE=1 and OA=0 whereas the remaining clinical data were continuous variables.

QC and selection of WGCNA modules were performed as follows. WGCNA modules of interest chosen for further analyses underwent a QC and selection process to ensure modules were reflective of disease state. In one component of this process, ME expression per patient was visually inspected to assess consistency of expression of patients in a given cohort. Second, module membership, also known as eigengene-based connectivity (kME) was plotted against probe correlation to the primary clinical trait of interest (SLEDAI) to gauge how well the genes in a given module agreed to the clinical trait. Finally, the Pearson correlations of MEs to the clinical metadata were examined. Absolute values of correlation coefficients in the range of 0.5 to 1 were considered strong, and p values<0.05 were considered significant. These three aspects of WGCNA taken together were used to identify modules for additional study.

Functional analysis was performed as follows. Immune/Inflammation-Scope (I-Scope) and Biologically Informed Gene Clustering (BIG-C) are functional aggregation tools for characterizing immune cells by type and biologically classifying large groupings of genes, respectively. I-Scope categorizes gene transcripts into a possible 32 hematopoietic cell categories based on matching 926 transcripts known to mark various types of immune/inflammatory cells. BIG-C sorts genes into 52 different groups based on their most probable biological function and/or cellular/subcellular localization. Tissue-Scope (T-Scope) is an additional aggregation tool to characterize cell types found in specific tissues. Transcripts are sorted into one of 8 categories representing a specific tissue or tissue cell subtype based on matching 704 total T-Scope transcripts. In these analyses only the two T-Scope categories relevant to the synovium were used: fibroblasts and synoviocytes.

Statistical analysis was performed as follows. Enrichment statistics were calculated by Fisher's Exact Test in R with the function fisher.test( ) and alternative hypothesis=“greater” to ensure the p-value was in the upper 5% of the probability distribution, i.e. that the true odds ratio is greater than 1 and the sample is enriched.

Ingenuity® Pathway Analysis (IPA) was performed as follows. The canonical pathway and upstream regulator functions of IPA core expression analysis tool (Qiagen) were used to interrogate DE data and gene lists from WGCNA modules. Core expression analyses were based on fold change if uploaded genes were differentially expressed; otherwise, a fold change of one was used. Canonical pathways and upstream regulators were considered significant if |Activation Z-Score|≥2 and overlap p-value<0.01.

Gene Set Variation Analysis (GSVA) was performed as follows. The GSVA R package was used as a non-parametric, unsupervised gene set enrichment (GSE) method. Enrichment scores were calculated using a Kolgomorov Smirnoff (KS)-like random walk statistic to estimate variation of pre-defined gene sets. The inputs for the GSVA algorithm were a gene expression matrix of log 2 microarray expression values (Affy HGU133plus2 definitions) and pre-defined gene sets co-expressed in SLE datasets. Log 2-transformed expression values were compiled into e-sets and low-intensity probes filtered out based on interquartile range (IQR). Probe density over a range of IQR values was plotted, and a threshold was selected at the IQR value corresponding to the maximum number of genes in the log 2-binned histogram. Probes below this characteristic IQR threshold were filtered out. GSVA was conducted on the remaining network and Welch's t-test was used to detect significant difference in enrichment between cohorts, followed by calculation of Hedge's g effect size with correction for small samples.

Enrichment gene sets containing cell type- and process-specific genes were created through an iterative process of identifying DE transcripts pertaining to a restricted profile of hematopoietic cells in 13 SLE microarray datasets and checked for expression in purified T cells, B cells, and monocytes to remove transcripts indicative of multiple cell types. Genes were identified through literature mining, GO biological pathways, and Search Tool for the Retrieval of Interacting Genes/Proteins (STRING) interactome analysis as belonging to specific categories. Enrichment gene sets were also created from IPA canonical pathways. Molecules from SLE vs OA synovium DE data that overlapped with the IPA signaling pathways of interest and were predicted to be upregulated in the pathway were included in the respective gene set. Select gene sets (e.g., TNF-induced, IFNβ-upregulated, M1, and M2 signatures) were derived directly from in vitro experiments. The M1 signature was edited to remove interferon genes. Additionally, IL-1 and IL-6 gene sets were derived from the first three tiers of the respective PathCards signaling pathways.

Library of Integrated Network-Based Cellular Signatures (LINCS) drug-target prediction and biological upstream regulator analysis was performed as follows. The Library of Integrated Network-Based Cellular Signatures (LINCS) perturbation database was queried using a DEG list of significantly upregulated and downregulated genes from the SLE and OA samples. The database was accessed at data.lincscloud.org.s3.amazonaws.com/index.html and contains the transcriptional responses from a compendium of perturbation experiments measured by the L1000 assay using Luminex Flexmap 3D bead technology. Over 25 different cell lines were antagonized by a vast array of pertubagens including drug compounds and chemicals, gene overexpression constructs, and gene silencing constructs. Queries of the lupus synovitis gene signature were compared to gene profiles in the LINCS database to predict potentially efficacious therapies and to predict significant dysregulation by specific gene products, termed biological upstream regulators (BURs). Comparisons were made based on LINCS-computed connectivity scores, where −100 describes a transcriptional program perfectly opposing the user-uploaded gene signature and 100 describes a transcriptional program perfectly representative of the user-uploaded gene signature.

Drug-target matching was performed as follows. In addition to the LINCS-predicted compounds, LINCS-predicted biological upstream regulators and IPA®-predicted upstream regulators were annotated with respective targeting drugs and compounds to elucidate potential useful therapies in lupus synovitis. Drugs targeting gene products of interest both directly and indirectly were sourced by IPA, the Connectivity Map via the drug repurposing tool, GeneCards, STITCH (V5.0), CoLTS-scored drugs, LINCS/CLUE databases, FDA labels, DrugBank, literature mining, and queries of clinical trials databases. Similar methods were employed to determine information about drugs, including mechanism of action and stage of clinical development.

STITCH analysis was performed as follows. The Search Tool for Interactions of Chemicals (STITCH) (V5.0) database of known and predicted protein-protein and protein-chemical interactions was used to predict direct and indirect drug targeting mechanisms. For each gene product of interest, the top 10 interactors were analyzed and drugs directly targeting the top interactors were matched according to the methods described. A medium confidence score cutoff of 0.4 for STITCH protein-protein or protein-chemical interaction predictions was used. Predicted interactions based solely on text-mining were not considered. The database was accessed at stitch.embl.de/.

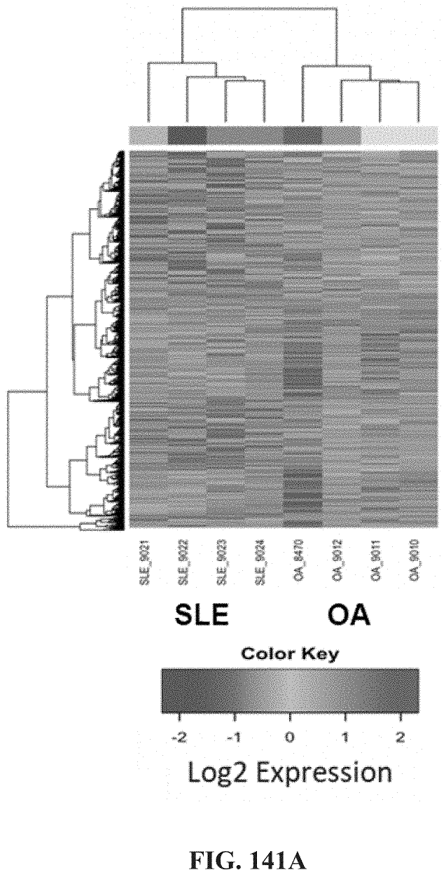

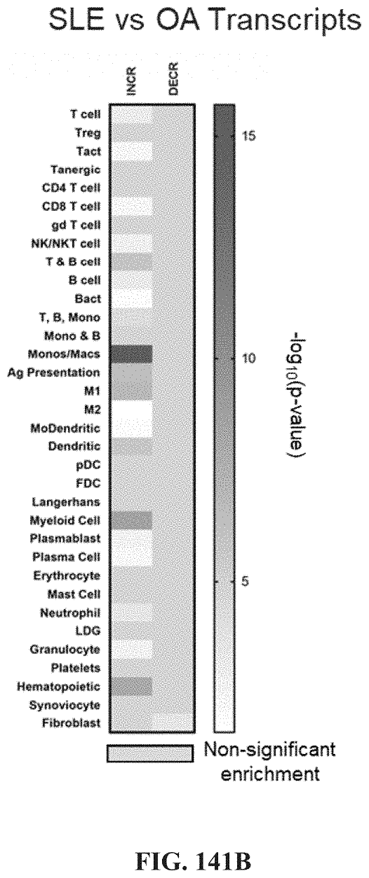

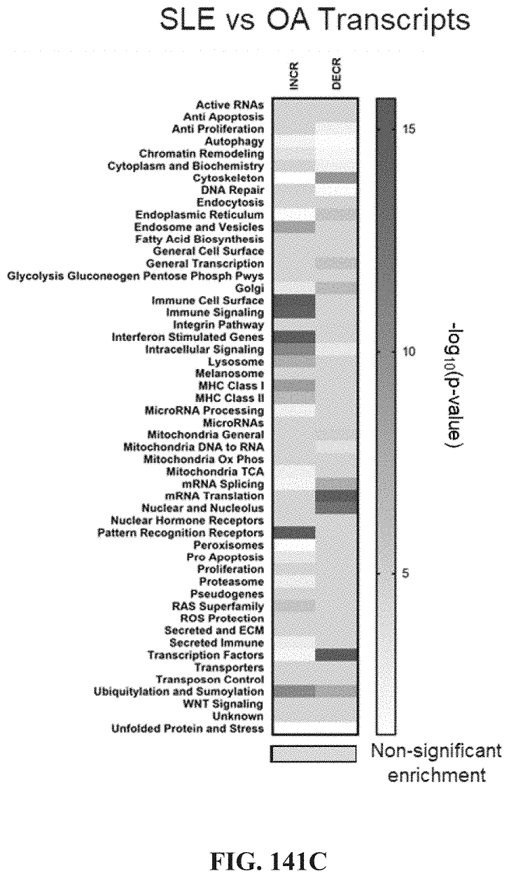

Bioinformatic analysis of SLE and OA synovitis gene expression was performed as follows. Comparison of synovial microarray data from SLE and OA subjects demonstrated a total of 6,496 DEGs with an FDR<0.2 (FIG. 141A). Of these, 2,477 transcripts were found to be upregulated whereas 4,019 transcripts were downregulated. To investigate the nature of these DEGs, I-Scope and T-Scope were used to determine significant immune/inflammatory or tissue-specific cell populations in SLE or OA. The upregulated DEGs included 243 I-Scope cell-specific transcripts (odds ratio of 2.84, p<2.2E-16, Fisher's Exact Test). Thus, the upregulated transcripts in lupus synovium represented a significant immune infiltrate and were interrogated further for specific I-Scope cell categories as well as for fibroblast and synoviocyte categories using T-Scope (FIG. 141B). These analyses revealed a significant enrichment of T-cell, B-cell, and myeloid cell transcripts among the upregulated DEGs. Specifically, antigen presenting cell markers were significantly enriched along with myeloid cells and monocytes/macrophages, comprised of mainly M1-polarized cells. Accordingly, BIG-C analysis revealed significant enrichment of immune functions including immune signaling and immune cell surface markers, the interferon transcriptional program, pattern recognition receptors (PRRs), and MHC Class I and II (FIG. 141C). Other functions related to intracellular signaling and processing/packaging material inside cells were also significantly enriched.

Of the 4,019 downregulated DEGs, only 17 overlapped with I-Scope transcripts, and thus downregulated genes did not reflect a change in immune/inflammatory cells (odds ratio of 0.0749, p=1). However, a significant number of DEGs identifying fibroblasts were downregulated in lupus synovium (FIG. 141B). BIG-C analysis identified several molecular processes nonspecific to one cell type that were decreased in lupus synovium (FIG. 141C). A list of genes significantly up- and downregulated in lupus synovium can be found in Table 73.

TABLE 73
DE genes identified as up-regulated or down-regulated
in synovium SLE vs. OA and in synovium RA vs. OA

| | |
|---|---|
| Up- | 36951, 38412, 38777, AAED1, AAK1, AASDH, AASDHPPT, AATBC, |
| regulated | ABCA1, ABCD1, ABCD3, ABCG2, ABHD10, ABHD12, ABHD13, |
| in | ABHD15, ABHD2, ABI1, ABI3, ABI3BP, ABT1, ABTB1, ACAA2, ACAP2, |
| Synovium | ACBD3, ACBD5, ACE, ACOT7, ACOX1, ACP2, ACSL5, ACSM5, ACTB, |
| SLE vs. | ACTR2, ACTR3, ACVR1B, ACVR2B, ADAM10, ADAM17, ADAM28, |
| OA | ADAM8, ADAMDEC1, ADAMTS5, ADAMTSL4, ADAP1, ADAR, |
| | ADCK2, ADCK3, ADD3, ADGRE2, ADGRE5, ADH5, ADNP2, ADPGK, |
| | ADRB2, ADRBK1, ADRBK2, AFF1, AGA, AGGF1, AGO1, AGO2, AGO3, |
| | AGPAT3, AGPAT5, AGPS, AGRN, AGTRAP, AHNAK, AHR, AIF1, AIM1, |
| | AIM2, AIP, AK4, AK9, AKAP13, AKAP8L, AKIRIN1, AKIRIN2, ALCAM, |
| | ALDH3B1, ALG10, ALKBH8, ALOX5, ALYREF, AMPD3, ANGPT1, |
| | ANGPTL1, ANKRD11, ANKRD12, ANKRD32, ANKRD36B, ANKRD44, |
| | ANP32A, ANP32E, ANXA4, ANXA7, AOAH, AP1AR, AP1G1, AP1S2, |
| | AP2A1, AP5M1, APC, APH1A, APH1B, API5, APIP, APOB, APOBEC3A, |
| | APOBEC3G, APOL1, APOL2, APOL3, APOL4, APOL6, APOOL, APPBP2, |
| | APPL1, AQP9, ARF1, ARF6, ARFGEF2, ARFIP1, ARGLU1, ARHGAP1, |
| | ARHGAP18, ARHGAP19, ARHGAP24, ARHGAP25, ARHGAP26, |
| | ARHGAP30, ARHGAP31, ARHGAP4, ARHGAP9, ARHGDIA, ARHGDIB, |
| | ARHGEF10L, ARHGEF2, ARHGEF3, ARHGEF7, ARID1A, ARID2, |
| | ARID4A, ARID4B, ARIH1, ARL11, ARL4D, ARL6IP1, ARL6IP5, |
| | ARL6IP6, ARL8A, ARL8B, ARMC7, ARNT, ARNTL2, ARPC2, ARPC4, |
| | ARPC5, ARPP19, ARRB2, ARSB, ARSK, ASAP1, ASAP1-IT1, ASB13, |
| | ASCC1, ASCL2, ASH1L, ASNA1, ASPH, ASPHD2, ASXL2, ATAD2, |
| | ATAD2B, ATF1, ATF2, ATF5, ATF6, ATF7, ATG16L2, ATG7, ATP10B, |
| | ATP2A3, ATP2B1, ATP5C1, ATP6V1A, ATP6V1B2, ATP6V1F, |
| | ATP6V1G1, ATP6V1H, ATP7A, ATXN2L, ATXN7, AUH, AVPI1, AZIN1, |
| | B2M, B3GALT4, B3GNT2, BACH1, BAG4, BAG5, BATF2, BAχ, BAZ1A, |
| | BAZ2A, BBIP1, BBX, BCAP29, BCAT1, BCL10, BCL11A, BCL2A1, |
| | BCL2L1, BCL2L11, BCL2L13, BCLAF1, BEX5, BHLHE41, BID, BIN2, |
| | BIRC3, BISPR, BLM, BLNK, BLOC1S1, BLVRA, BLVRB, BMP2K, |
| | BNIP3L, BPTF, BRAF, BRCC3, BRD3, BRD7, BROX, BRSK1, BSG, BST2, |
| | BTC, BTD, BTF3L4, BTG2, BTK, BTN2A2, BTN3A1, BTN3A3, BUB3, |
| | C10orf10, C10orf113, C10orf62, C10orf90, C11orf58, C11orf71, C12orf4, |
| | C12orf5, C12orf66, C14orf159, C14orf28, C15orf48, C16orf54, C16orf72, |
| | C17orf80, C19orfl2, C19orf43, C19orf66, C19orf84, C1D, C1orf162, |
| | C1orf27, C1orf94, C1QA, C1QB, C1QC, C1S, C2, C21orf91, C22orf15, |
| | C2orf47, C2orf49, C2orf81, C3AR1, C3orf38, C5orf22, C5orf56, C6orf106, |
| | C6orf62, C6orf89, C9orf72, C9orf91, CA5B, CAB39, CABP4, CACUL1, |
| | CALCRL, CALM1, CALR, CAMK1D, CAMK2D, CAMSAP1, CANX, |
| | CAPG, CAPRIN1, CAPZA2, CARD16, CARD8-AS1, CASP1, CASP10, |
| | CASP2, CASP8, CAST, CBFB, CBL, CBLB, CBR3, CBX4, CBX5, |
| | CC2D2A, CCDC112, CCDC146, CCDC174, CCDC186, CCDC28A, |
| | CCDC47, CCDC50, CCDC59, CCDC6, CCDC60, CCDC88A, CCL23, |
| | CCL4, CCL5, CCL8, CCND3, CCNDBP1, CCNG2, CCNT1, CCNYL1, |
| | CCR1, CCR2, CCR5, CCRL2, CD163, CD164, CD19, CD200R1, CD27, |
| | CD274, CD2AP, CD37, CD38, CD3D, CD4, CD40, CD44, CD46, CD47, |
| | CD48, CD52, CD53, CD58, CD6, CD70, CD72, CD74, CD80, CD83, CD84, |
| | CD86, CD8A, CD8B, CD99P1, CDC27, CDC40, CDC42, CDC42EP3, |
| | CDC42-IT1, CDC42SE2, CDC5L, CDK12, CDK17, CDK19, CDK6, |
| | CDK9, CDKN1B, CDKN2A, CDKN2AIP, CDS2, CDT1, CDV3, CDYL, |
| | CECR1, CENPQ, CEP104, CEP350, CEP57, CEP68, CEPT1, CERS6, CFL1, |
| | CFLAR, CFP, CGGBP1, CHAF1A, CHCHD3, CHD1, CHD2, CHFR, CHM, |
| | CHMP1B, CHMP4B, CHMP5, CHN2, CHP1, CHPT1, CHST11, CHURC1, |
| | CIDEB, CISH, CITED2, CKLF, CLASP2, CLCN3, CLCN7, CLEC12A, |
| | CLEC2B, CLEC7A, CLIC2, CLIC4, CLINT1, CLIP4, CLK3, CLMN, CLUH, |
| | CMKLR1, CMPK2, CMTM6, CMTR1, CNBP, CNDP2, CNEP1R1, CNNM3, |
| | CNOT1, CNOT6L, CNOT7, CNPY3, CNTRL, COCH, COL11A2, |
| | COL4A3BP, COL6A1, COLGALT1, COMT, COPA, COQ10B, COQ7, |
| | CORO1A, CORO1C, CORO2A, COX15, COX6A1, CP, CPEB2, CPEB3, |
| | CPEB4, CPM, CPNE5, CPNE8, CPPED1, CPVL, CRADD, CREB1, |
| | CREB3L2, CREBL2, CREBRF, CRIM1, CRLF3, CRLS1, CRTAM, CSF1, |
| | CSF1R, CSF2RA, CSF2RB, CSF3R, CSGALNACT2, CSNK1G1, |
| | CSNK2A1, CST7, CSTB, CSTF2T, CTBP1-AS2, CTBS, CTDSPL2, |
| | CTNNA1, CTSB, CTSC, CTSH, CTSO, CTSS, CTSW, CTSZ, CTTNBP2NL, |
| | CUEDC1, CUL7, CWC15, CX3CL1, CXCL10, CXCL11, CXCL13, |
| | CXCL16, CXCL2, CXCL3, CXCL8, CXCL9, CXCR3, CXCR4, CXorf21, |
| | CXorf38, CYB5A, CYB5B, CYB5R3, CYB5R4, CYBA, CYBB, CYBRD1, |
| | CYLD, CYP1A2, CYP1B1, CYP20A1, CYP27A1, CYP2S1, CYSLTR1, |
| | CYTH1, CYTH4, CYTIP, DAPK1, DAPK1-IT1, DAPP1, DAZAP2, DBI, |
| | DBP, DCAF11, DCAF7, DCBLD1, DCK, DCP1A, DCP2, DCTN4, DCTN5, |
| | DCTN6, DCUN1D1, DDA1, DDHD1, DDIT4, DDX17, DDX18, DDX24, |
| | DDX58, DDX60, DDX60L, DEDD, DEDD2, DEF6, DEK, DENND1B, |
| | DENND3, DENND4A, DENND5A, DENR, DERA, DERL1, DERL3, |
| | DGKH, DHRS12, DHRS7, DHRS9, DHX36, DHX9, DIAPH1, DICER1, |
| | DIP2B, DIRC2, DISC1, DLAT, DLD, DLG1, DMBT1, DMXL2, DNAH3, |
| | DNAJA2, DNAJB14, DNAJB9, DNAJC14, DNAJC17, DNAJC3, DNAJC5B, |
| | DNMT3A, DNTTIP1, DOCK10, DOCK2, DOCK5, DOCK8, DOK2, DPF2, |
| | DPM1, DPY30, DR1, DSC2, DSCR3, DTWD2, DTX3L, DUSP1, DUSP10, |
| | DUSP11, DUSP3, DUSP7, DUSP8, DYNC1H1, DYNLT1, DYRK2, E2F3, |
| | EAF2, EBAG9, EBP, ECE1, ECHDC1, EFCAB14, EFR3A, EGFR, EGLN1, |
| | EGLN3, EHMT2, EIF1B, EIF2AK2, EIF2S1, EIF4E, EIF4E3, EIF4G1, |
| | EIF5A, ELF1, ELF4, ELK4, ELMSAN1, EMB, EMC4, EMILIN2, EMILIN3, |
| | EML4, EMP3, ENC1, ENDOV, ENG, ENO1, ENSA, ENTPD4, ENTPD5, |
| | EPB41L2, EPB41L3, EPHB2, EPOR, EPRS, EPS15L1, EPSTI1, ERAP1, |
| | ERC1, ERGIC1, ERICH1, ERLIN1, ERO1L, ERP27, ERP29, ESCO1, |
| | ETNK1, ETS1, ETV3, ETV5, ETV6, ETV7, EVI2A, EVI2B, EWSR1, |
| | EXOC4, EXOC5, EXOC7, EZR, FUR, FAF2, FAM103A1, FAM105A, |
| | FAM107A, FAM107B, FAM117A, FAM117B, FAM120AOS, FAM122A, |
| | FAM129A, FAM134A, FAM13A, FAM175B, FAM184A, FAM188A, |
| | FAM192A, FAM198B, FAM199X, FAM204A, FAM26F, FAM46C, |
| | FAM49B, FAM60A, FAM76A, FAM78A, FAM91A1, FAM96A, FANCD2, |
| | FANCF, FAR1, FAR2, FBXL3, FBXL4, FBXO28, FBXO38, FBXO44, |
| | FBXO8, FCER1G, FCGR2A, FCGR2C, FCGR3B, FCHO2, FCN1, FCRLA, |
| | FCRLB, FEM1B, FERMT3, FEZ2, FGD2, FGD3, FGD4, FGD6, FGF10, |
| | FGFR1OP2, FGR, FICD, FIG. 4, FKBP15, FKBP1A, FKBP5, FKBP8, |
| | FKSG49, FLCN, FLU, FLJ10038, FLJ20021, FLOT2, FLT3LG, FLVCR2, |
| | FMNL2, FMO2, FN1, FNBP1, FNDC3A, FNDC3B, FNIP1, FOLR1, FOLR3, |
| | FOPNL, FOXD1, FOXN2, FPR3, FRAT1, FRMD4A, FRMD4B, FRYL, |
| | FTH1P5, FUOM, FUS, FXR1, FYB, FYTTD1, FZD5, FZD6, G2E3, G3BP1, |
| | G3BP2, GAB1, GAD1, GALC, GALM, GALNT6, GALNT7, GAS2L3, |
| | GAS7, GAS8, GATA3, GBGT1, GBP1, GBP1P1, GBP2, GBP4, GBP5, |
| | GCA, GCH1, GCLC, GCNT1, GDAP2, GDE1, GFI1, GFPT1, GFRA2, |
| | GGA2, GGA3, GIGYF1, GIMAP1, GIMAP2, GIMAP4, GIMAP6, GIMAP7, |
| | GIT2, GJD3, GK, GLG1, GLIPR1, GLIPR2, GLTSCR1L, GLUD1, GLUD2, |
| | GLUL, GM2A, GMEB1, GMFB, GMFG, GMPR, GNA13, GNAS, GNB1, |
| | GNB4, GNB5, GNG2, GNG5, GNRH1, GNS, GOLGA7, GOLIM4, |
| | GOLPH3L, GON4L, GPANK1, GPATCH11, GPD2, GPN3, GPNMB, |
| | GPR132, GPR155, GPR160, GPR183, GPR22, GPRIN3, GPSM3, GRB2, |
| | GRK6, GRN, GSE1, GSK3B, GSN, GSTK1, GSTO1, GSTTP1, GTF2B, |
| | GTF2F1, GTF2I, GTPBP8, GULP1, GVINP1, GXYLT1, GYG1, GZMA, |
| | GZMH, H1F0, H2AFJ, H2AFY, H2BFS, HACD2, HACL1, HAGHL, |
| | HAUS1, HAUS2, HAVCR2, HBP1, HCCS, HCG18, HCLS1, HCP5, HCRP1, |
| | HCST, HECTD1, HELLS, HELZ2, HERC5, HERC6, HERPUD2, HEXA, |
| | HFE, HHEX, HIAT1, HIATL2, HIF3A, HIPK1, HIPK2, HIPK3, HIST1H1C, |
| | HIST1H2BD, HIST1H4C, HIST2H2BE, HIST3H2A, HLA-A, HLA-B, HLA- |
| | C, HLA-DMA, HLA-DMB, HLA-DOA, HLA-DOB, HLA-DPA1, HLA- |
| | DPB1, HLA-DQB1, HLA-DQB2, HLA-DRA, HLA-DRB1, HLA-DRB6, |
| | HLA-E, HLA-F, HLA-G, HLA-J, HM13, HMGA1, HMGB1, HMGB2, |
| | HMGXB3, HNMT, HNRNPA0, HNRNPD, HNRNPF, HNRNPH1, |
| | HNRNPK, HNRNPLL, HNRNPR, HNRNPUL1, HOXB-AS3, HPCAL1, |
| | HPS1, HPS5, HPSE, HS3ST5, HSD11B1, HSD17B11, HSD17B14, |
| | HSD17B4, HSD17B8, HSH2D, HSPA13, HSPA4, HTR4, HVCN1, IAH1, |
| | ICAM1, ICE2, ICOS, IDE, IDH1, IDO1, IDS, IFI16, IFI27, IFI27L1, IFI35, |
| | IFI44, IFI44L, IFI6, IFIH1, IFIT1, IFIT2, IFIT3, IFIT5, IFITM1, IFNAR2, |
| | IFNGR1, IGF1R, IGF2BP3, IGF2R, IGFBP5, IGHD, IGHG1, IGHM, |
| | IGHV1-2, IGHV1-46, IGHV3-21, IGHV3-23, IGHV4-31, IGHV4-34, IGK, |
| | IGKC, IGKV1D-8, IGKV4-1, IGLC1, IGLJ3, IGLV1-40, IGLV1-44, IGLV2- |
| | 14, IGLV3-10, IGLV3-19, IGLV3-25, IGLVI-70, IGSF6, IKBKE, IKZF1, |
| | IKZF5, IL10, IL10RA, IL12RB1, IL15, IL15RA, IL16, IL17RA, IL18BP, |
| | IL18RAP, IL1A, IL1R1, IL1RN, IL21R, IL27RA, IL2RG, IL32, IL4I1, IL6R, |
| | IL6ST, IL7R, IMPA1, INAFM2, ING2, ING3, INIP, INO80B, INO80D, |
| | INPP5D, INTS6, IQCK, IQGAP1, IQGAP2, IRAK3, IREB2, IRF1, IRF2, |
| | IRF2BP2, IRF2BPL, IRF4, IRF5, IRF7, IRF8, IRF9, IRX2, ISCA1, ISCA2, |
| | ISG15, ISG20, ITCH, ITGA4, ITGA6, ITGA8, ITGAL, ITGAX, ITGB2, |
| | ITGB2-AS1, ITGB7, ITPK1, ITPK1-AS1, ITPKB, ITPR1, ITPRIPL2, JAK1, |
| | JAK2, JAZF1, JMJD1C, JUN, JUND, JUP, KANSL1, KAT2B, KAT6B, |
| | KATNBL1, KBTBD2, KCNAB2, KCNE3, KCNH6, KCNQ1, KCNRG, |
| | KCTD11, KCTD12, KCTD20, KCTD5, KDM2A, KDM3A, KDM5A, |
| | KDM6A, KDM6B, KIAA0226, KIAA0907, KIAA1033, KIAA1143, |
| | KIAA1147, KIAA1551, KIAA2026, KIDINS220, KIF5B, KLF11, KLF12, |
| | KLF13, KLF3, KLF6, KLF9, KLHDC3, KLHDC7B, KLHL20, KLHL24, |
| | KLHL28, KLHL6, KLHL7, KLHL8, KLK12, KMO, KMT2C, KMT2E, |
| | KPNA3, KRAS, KRCC1, KRT78, KYNU, LACC1, LACTB, LACTB2, |
| | LAIR1, LAIR2, LAMP2, LAMP3, LAP3, LAPTM5, LARP1B, LARP4, |
| | LARP4B, LAT, LAX1, LCK, LCN6, LCOR, LCORL, LCP1, LCP2, LEMD3, |
| | LEPROT, LEPROTL1, LGALS2, LGALS3, LGALS3BP, LGALS9, LGI4, |
| | LIFR, LILRA1, LILRA2, LILRA5, LILRA6, LILRB1, LILRB2, LILRB3, |
| | LILRB4, LIMA1, LIMD1, LIME1, LIMS1, LIN7C, LIN9, LINC00467, |
| | LINC00537, LINC00657, LINC00847, LINC00936, LINC00957, |
| | LINC01278, LINC01578, LMAN1, LMAN2, LMO4, LNPEP, |
| | LOC100129029, LOC100130100, LOC100190986, LOC100506990, |
| | LOC100507477, LOC100630918, LOC100996251, LOC100996740, |
| | LOC101926887, LOC101927027, LOC101927402, LOC101927699, |
| | LOC101928728, LOC102724699, LOC202181, LOC286238, LOC374443, |
| | LOC441025, LOC647070, LPCAT2, LPXN, LRCH1, LRCH3, LRIF1, |
| | LRMP, LRP10, LRP8, LRRC16A, LRRC40, LRRC8C, LRRC8D, LRRFIP1, |
| | LRRTM2, LSM12, LSM14B, LSR, LST1, LTA4H, LTBP4, LTN1, LY6E, |
| | LY6K, LY75, LY9, LY96, LYL1, LYN, LYPLA1, LYPLA2, LYSMD2, |
| | LYST, LZIC, LZTFL1, M6PR, MAF, MAFB, MALAT1, MAML2, |
| | MAN1A1, MAP2K1, MAP2K6, MAP3K1, MAP3K2, MAP3K5, MAP4K1, |
| | MAPK1, MAPK14, MAPK3, MAPKAP1, MAPKAPK3, MAPRE1, MAPT- |
| | AS1, MARCKS, MARCO, MARK4, MAT2B, MATK, MAX, MAZ, MBD1, |
| | MBD2, MBD4, MBLAC1, MBNL1, MBNL2, MBP, MCEMP1, MCFD2, |
| | MCL1, MCOLN2, MCTP1, MDM2, MED1, MED13L, MED19, MED30, |
| | MED6, MEF2C, MEGF9, MEI1, MET, METTL14, METTL7A, MFHAS1, |
| | MFNG, MFSD6, MGA, MGAT1, MGAT2, MGAT4A, MGEA5, MIB1, |
| | MICB, MICU1, MID1IP1, MIER1, MIER3, MIF4GD, MIS18A, MIS18BP1, |
| | MKRN2, MLEC, MLLT10, MLX, MMP1, MMP24-AS1, MNDA, MOB1A, |
| | MON2, MORC2-AS1, MORF4L1, MPC1, MPP5, MPZL1, MR1, MREG, |
| | MROH1, MRPL30, MRPL42, MRPL44, MRPL49, MRPS10, MRPS18A, |
| | MRPS18B, MRPS18C, MRPS22, MRPS31, MS4A4A, MS4A6A, MS4A7, |
| | MSANTD4, MSI2, MSL2, MSR1, MT1M, MTDH, MTF2, MTHFD2, |
| | MTHFR, MTM1, MTMR14, MTPAP, MTSS1, MTUS1, MX1, MX2, MXD1, |
| | MXI1, MYCBP2, MYD88, MYEF2, MYL12A, MYO19, MYO1F, MYO5A, |
| | MZB1, N4BP1, N4BP2L1, N6AMT1, NAA15, NAA50, NAAA, NADK, |
| | NAPA, NAPG, NASP, NAT1, NBEAL1, NBEAL2, NBN, NBR1, NCF2, |
| | NCF4, NCK1, NCK1-AS1, NCKIPSD, NCOA2, NCOA3, NCOA4, NCOA7, |
| | ND2, NDE1, NDFIP1, NDOR1, NDRG2, NDUFA12, NDUFA7, NDUFAB1, |
| | NDUFB8, NDUFS1, NDUFV2, NECAP2, NEDD9, NEK1, NEK7, NFAM1, |
| | NFAT5, NFE2L2, NFE2L3, NFIA, NFKB2, NFKBIA, NFKBIE, NFKBIZ, |
| | NFX1, NFYC, NGDN, NHSL1, NIPAL2, NIPAL3, NIPBL, NKAPP1, NKG7, |
| | NKTR, NLK, NLRC5, NLRP12, NLRP3, NMI, NMRK1, NMT1, NNT, |
| | NOL7, NONO, NPAT, NPC1, NPM2, NPTN, NR1D2, NR1H2, NR3C1, |
| | NRAS, NREP, NRIP1, NRIP3, NRP2, NRROS, NSFL1C, NT5C3A, NUAK2, |
| | NUB1, NUCKS1, NUDT17, NUDT21, NUMA1, NUP153, NUP210, NUP50, |
| | NUTF2, NXPE3, NXT2, OAS1, OAS2, OAS3, OASL, ODF3B, OGFR, |
| | OGFRL1, OGT, OPTN, OR7A10, ORC3, ORMDL1, OSBP, OSBPL11, |
| | OSBPL3, OSBPL8, OSBPL9, OSER1, OSGEPL1, OSGIN2, OSTF1, |
| | OSTM1, OTUD1, OTUD5, OXNAD1, P2RX1, P2RX7, P2RY12, P2RY8, |
| | PACSIN2, PAG1, PAIP2, PAK1, PAK2, PALB2, PALLD, PAPD4, |
| | PAPOLA, PAPOLG, PAPPA2, PAPSS1, PAPSS2, PAQR4, PAQR8, |
| | PARP10, PARP11, PARP12, PARP14, PARP4, PARP8, PARP9, PARVG, |
| | PATL1, PBRM1, PBX2, PCBD2, PCBP1, PCBP2, PCDHGB5, PCGF1, |
| | PCGF5, PCIF1, PCMTD1, PCMTD2, PCNP, PCYOX1, PDCD10, |
| | PDCD1LG2, PDE12, PDE4DIP, PDE6D, PDGFC, PDK1, PDLIM5, PDP1, |
| | PDXDC1, PEA15, PECAM1, PEG3, PELI1, PEX13, PEX16, PEX3, PFN1, |
| | PGGT1B, PGK1, PGRMC1, PHACTR2, PHC3, PHF11, PHF3, PI4K2A, |
| | PI4K2B, PIAS1, PIAS2, PICALM, PIGO, PIK3AP1, PIK3C2A, PIK3CB, |
| | PIK3CD, PIK3CG, PIK3IP1, PIK3R1, PILRA, PIP4K2A, PIP5K1A, PKN2, |
| | PLCB1, PLCG1, PLCG2, PLCL2, PLD3, PLEC, PLEK, PLEKHA3, |
| | PLEKHB2, PLEKHF2, PLEKHG3, PLEKHO1, PLSCR1, PLXDC2, |
| | PMFBP1, PML, PNISR, PNKD, PNPLA8, PNPT1, PNRC1, PNRC2, POC5, |
| | POGK, POLB, POLD3, POLDIP3, POLH, POLK, POLR1D, POLR2E, |
| | PPA1, PPFIA1, PPIG, PPM1A, PPM1D, PPM1K, PPM1L, PPP1CB, |
| | PPP1R10, PPP1R12A, PPP1R12B, PPP1R15A, PPP1R2, PPP1R3D, PPP2CB, |
| | PPP2R5C, PPP3CB-AS1, PPP3R1, PPP4C, PPT1, PPTC7, PRB1, PRDM1, |
| | PRDX3, PREX1, PRF1, PRIM1, PRIM2, PRKACB, PRKAG2, PRKCB, |
| | PRKCD, PRKCI, PRKD3, PRKRIR, PRNP, PRODH, PRPF4, PRPF40A, |
| | PRPS2, PRRC1, PRRC2C, PRRG4, PSAP, PSD4, PSEN1, PSENEN, PSIP1, |
| | PSKH1, PSMA3, PSMA5, PSMA6, PSMB10, PSMB3, PSMB4, PSMB8, |
| | PSMB8-AS1, PSMB9, PSMC2, PSMD14, PSME1, PSME3, PSME4, PSMF1, |
| | PSTPIP2, PTAFR, PTBP3, PTER, PTGER2, PTGER4, PTGES, PTGR1, |
| | PTK2B, PTK6, PTMS, PTP4A1, PTP4A2, PTPN1, PTPN12, PTPN4, PTPN6, |
| | PTPN7, PTPRA, PTPRC, PTPRCAP, PTPRJ, PTPRO, PUS10, PUS7L, PVR, |
| | PVRIG, PVRL2, PXK, PXMP4, PYCARD, PYHIN1, QKI, QPCT, QSER1, |
| | RAB10, RAB11FIP1, RAB12, RAB14, RABI8, RAB20, RAB23, RAB2A, |
| | RAB30, RAB35, RAB3D, RAB3GAP1, RAB3IL1, RAB4B, RAB5A, |
| | RAB5C, RAB8A, RAB8B, RAB9A, RABGAP1L, RABL3, RAC1, RAC2, |
| | RAD21, RAD23A, RAD51AP1, RALA, RALB, RANBP3, RAP1B, |
| | RAP1GDS1, RAP2A, RAP2B, RAP2C, RAPGEF2, RARG, RARRES3, |
| | RARS2, RASA2, RASAL2, RASGRP1, RASGRP2, RASSF2, RASSF3, |
| | RASSF4, RASSF5, RASSF7, RB1, RB1CC1, RBBP4, RBBP5, RBCK1, |
| | RBKS, RBL1, RBL2, RBM12B, RBM22, RBM25, RBM27, RBM39, |
| | RBM43, RBM47, RBM5, RBMS3, RBMXL1, RBMY3AP, RC3H2, RCHY1, |
| | RCSD1, RDM1, RDX, REC8, RECQL, REEP5, REL, RELT, RENBP, RERE, |
| | RFC3, RFC5, RFK, RFX2, RFX5, RGS1, RGS10, RGS14, RGS18, RGS5, |
| | RHBDD2, RHBDF2, RHEB, RHOA, RHOB, RHOH, RHOQ, RHOU, RIC1, |
| | RIF1, RILPL2, RIN3, RIOK3, RIPK1, RIPK2, RIT1, RLF, RMDN1, |
| | RMND5A, RNASE6, RNASET2, RNF114, RNF115, RNF125, RNF13, |
| | RNF130, RNF138, RNF14, RNF141, RNF144B, RNF149, RNF170, |
| | RNF19A, RNF19B, RNF207, RNF213, RNF38, RNF4, RNF44, RNGTT, |
| | RNPC3, ROCK1, ROCK2, RORA, RP2, RPS27A, RPS6KA1, RPS6KA2, |
| | RPS6KA3, RPS6KA5, RPS6KB1, RRAGD, RRAS, RRBP1, RSAD2, |
| | RSBN1L, RSF1, RSRC1, RSRC2, RTN4, RTP4, RUFY3, RUNX3, RXRA, |
| | RYBP, S100A11, S100A9, S1PR3, SALL4, SAMD9, SAMD9L, SAMHD1, |
| | SAMSN1, SAP18, SART3, SASH3, SAT1, SATB1, SAV1, SBF2, SCAF11, |
| | SCAF8, SCAMP1, SCAMP2, SCAND2P, SCAPER, SCARB1, SCARB2, |
| | SCN8A, SCOC, SCPEP1, SDAD1, SDC2, SDC3, SDCBP, SDCCAG3, |
| | SDE2, SDHA, SDHAF4, SDHB, SDHC, SDHD, SEC11C, SEC14L1, |
| | SEC22B, SEC22C, SEC61A1, SECISBP2L, SECTM1, SEL1L, SEL1L3, |
| | SELK, SELL, SELPLG, SELT, SEMA3C, SEMA4A, SEMA4D, SENP2, |
| | SEPSECS, SEPT11, SEPT2, SEPT7, SERBP1, SERINC3, SERP1, |
| | SERPINA1, SERPINB1, SERPINB9, SESTD1, SET, SETD8, SETDB2, |
| | SETX, SF3B1, SFMBT1, SFN, SFT2D1, SFT2D2, SFTPA2, SGK223, |
| | SGMS1, SGMS2, SGOL2, SGPL1, SGTB, SH2D3C, SH3BGRL, SH3BP1, |
| | SH3BP2, SH3BP5, SH3BP5L, SH3D19, SH3GLB2, SIAH1, SIGLEC1, |
| | SIGLEC10, SIGLEC7, SIGLEC9, SIKE1, SIMC1, SIRPA, SIRT1, SIT1, |
| | SKAP2, SKI, SKP1, SLA, SLAIN2, SLAMF7, SLAMF8, SLC11A2, |
| | SLC12A6, SLC12A9, SLC15A1, SLC15A3, SLC15A4, SLC16A4, |
| | SLC16A6, SLC17A5, SLC1A3, SLC1A4, SLC22A14, SLC25A24, |
| | SLC25A36, SLC25A37, SLC25A40, SLC29A3, SLC2A13, SLC2A5, |
| | SLC2A6, SLC30A1, SLC30A7, SLC31A2, SLC35D2, SLC35E2, SLC35E3, |
| | SLC35F6, SLC39A14, SLC39A8, SLC45A4, SLC4A7, SLC5A5, SLC7A8, |
| | SLC8A1, SLC8B1, SLCO2B1, SLFN5, SLIT2, SLMO2, SLN, SMAD5, |
| | SMAP2, SMC3, SMC5, SMCHD1, SMCR8, SMDT1, SMEK1, SMIM14, |
| | SMIM15, SMPDL3A, SNAP23, SNAP29, SNAPC3, SNAPIN, SNIP1, |
| | SNRNP27, SNRPB, SNTB1, SNW1, SNX1, SNX10, SNX13, SNX14, |
| | SNX15, SNX18, SNX2, SNX20, SNX27, SNX3, SNX6, SNX9, SOAT1, |
| | SOD2, SORT1, SOS2, SOX13, SOX5, SP1, SP100, SP110, SP140, |
| | SPATA13, SPATA24, SPCS2, SPCS3, SPEN, SPG11, SPI1, SPIN1, SPIRE1, |
| | SPN, SPOPL, SPPL2A, SPPL3, SPRED1, SPTLC1, SPTLC2, SQRDL, |
| | SQSTM1, SREBF2, SRFBP1, SRGAP2, SRGAP2C, SRGN, SRPK2, SRPR, |
| | SRRM1, SRSF3, SRSF7, SRSF9, SS18, SSBP1, SSH1, SSR1, ST6GAL1, |
| | ST6GALNAC4, ST8SIA4, ST8SIA5, STAC3, STAM2, STARD3NL, |
| | STARD5, STAT1, STAT2, STEAP3, STIM2, STIP1, STK10, STK17B, |
| | STK24, STK4, STOM, STON2, STRN3, STX11, STX16, STX17, STX7, |
| | STXBP2, STXBP3, SUB1, SUCNR1, SULT1A1, SUMO1, SUMO3, |
| | SUMO4, SUPT16H, SURF1, SVIP, SYF2, SYK, SYNCRIP, SYNGR2, |
| | SYNPO2, SYPL1, SZRD1, TAB2, TACC1, TAF1, TAF15, TAF1B, TAGAP, |
| | TAGLN2, TANK, TAOK1, TAP1, TAP2, TAPBP, TAPBPL, TBC1D12, |
| | TBC1D15, TBC1D5, TBC1D9, TBL1XR1, TCEB3, TCF20, TCF25, TCF4, |
| | TCN2, TDP2, TDRD7, TERF2, TES, TESK2, TET2, TET3, TFAM, TFE3, |
| | TFEC, TFPI, TGFBR2, TGFBRAP1, TGIF1, TGIF2, TGOLN2, TGS1, |
| | THAP6, THEMIS2, THRAP3, THUMPD1, THUMPD3, THUMPD3-AS1, |
| | TIFA, TIMP3, TINF2, TIPRL, TIRAP, TKT, TLE4, TLR1, TLR2, TLR3, |
| | TLR4, TLR6, TLR8, TM9SF2, TM9SF3, TMBIM4, TMBIM6, TMED2, |
| | TMED4, TMED5, TMED8, TMEM106A, TMEM123, TMEM128, |
| | TMEM140, TMEM159, TMEM170A, TMEM19, TMEM194A, TMEM206, |
| | TMEM229B, TMEM245, TMEM30A, TMEM33, TMEM37, TMEM41B, |
| | TMEM50A, TMEM53, TMEM55B, TMEM63B, TMEM70, TMEM86A, |
| | TMEM9B, TMF1, TMOD3, TMPO, TMSB10, TMX1, TMX4, TNFAIP2, |
| | TNFAIP3, TNFAIP8, TNFAIP8L2, TNFRSF10A, TNFRSF1B, TNFSF10, |
| | TNFSF13B, TNIP1, TNKS2, TNNI3, TNPO1, TNRC6B, TOB1, TOP1, |
| | TOR1AIP1, TOR1B, TOR4A, TOX4, TP53, TPCN2, TPD52, TPM4, TPP1, |
| | TPP2, TPR, TPRKB, TRA2B, TRAF3IP3, TRAK1, TRAM1, TRAM2, |
| | TRAPPC10, TRAPPC8, TRAV6, TRERF1, TREX1, TRG-AS1, TRIB1, |
| | TRIB3, TRIM13, TRIM14, TRIM21, TRIM22, TRIM23, TRIM25, TRIM33, |
| | TRIM38, TRIM6, TRIM66, TRIM69, TRIO, TRIOBP, TRMT1L, TRPS1, |
| | TRPV2, TSC22D3, TSN, TSPAN14, TSPAN5, TSTD1, TTC30B, TTC37, |
| | TTC9C, TTF1, TTR, TUBA3C, TUBA4A, TUBGCP2, TULP4, TWF1, |
| | TXNDC2, TXNDC8, TXNIP, TYMP, U2AF2, U2SURP, UBA6, UBALD2, |
| | UBB, UBC, UBD, UBE2A, UBE2B, UBE2D1, UBE2D3, UBE2D4, |
| | UBE2G1, UBE2H, UBE2J1, UBE2K, UBE2L6, UBE2N, UBE2W, UBE2Z, |
| | UBE3C, UBN1, UBN2, UBQLN1, UBQLN2, UBR2, UBXN11, UBXN4, |
| | UBXN6, UBXN7, UCHL5, UCP2, UEVLD, UFL1, UFM1, UGCG, UHMK1, |
| | UHRF1BP1, ULK4, UNC93B1, UQCRC2, USF2, USO1, USP1, USP10, |
| | USP15, USP16, USP18, USP25, USP3, USP30, USP30-AS1, USP33, USP38, |
| | USP8, USP9X, UST, UTP23, UTRN, UTY, UVRAG, VAC 14, VAMP2, |
| | VAMP3, VAMP5, VAMP8, VASH1, VASP, VIPAS39, VM01, VMP1, |
| | VNN2, VPS13B, VPS13C, VPS29, VPS33A, VPS8, VRK1, VRK2, VRK3, |
| | VTA1, VTI1A, WAC, WAPAL, WARS, WAS, WASF2, WASL, WBP11, |
| | WDFY1, WDFY2, WDFY3, WDFY4, WDR1, WDR48, WDR61, WDTC1, |
| | WEE1, WHSC1L1, WIPF1, WIPI2, WNK1, WSB1, WSB2, WTAP, WWP1, |
| | WWP2, XAB2, XAF1, XPO7, XRCC4, XRN1, XRN2, YBX1, YDJC, |
| | YME1L1, YPEL1, YPEL2, YPEL5, YTHDF3, YWHAE, YWHAH, |
| | YWHAZ, ZADH2, ZBTB1, ZBTB38, ZBTB4, ZBTB43, ZC3H12D, ZC3H4, |
| | ZC3H7B, ZC3HAV1, ZCCHC6, ZCCHC7, ZDHHC20, ZDHHC22, |
| | ZDHHC7, ZDHHC8, ZEB1, ZEB2, ZFAND5, ZFAND6, ZFP36L2, ZFR, |
| | ZFX, ZFYVE1, ZFYVE16, ZFYVE28, ZIK1, ZMIZ1-AS1, ZMYM2, |
| | ZMYND15, ZMYND8, ZNF101, ZNF124, ZNF134, ZNF142, ZNF148, |
| | ZNF200, ZNF207, ZNF22, ZNF24, ZNF254, ZNF274, ZNF277, ZNF333, |
| | ZNF33B, ZNF345, ZNF350, ZNF397, ZNF438, ZNF445, ZNF490, ZNF567, |
| | ZNF609, ZNF641, ZNF644, ZNF652, ZNF655, ZNF664, ZNF675, ZNF688, |
| | ZNF706, ZNF765, ZNF800, ZNF93, ZNFX1, ZSWIM6, ZWILCH, ZYG11B, |
| | ZYX |
| Down- | 37316, 37681, 39142, A2M, AACS, AADAT, AAGAB, AAMDC, AAR2, |
| regulated in | AARS, AASS, AATF, ABCB6, ABCC10, ABCC5, ABCD4, ABCE1, |
| Synovium | ABCF2, ABHD14B, ABHD17C, ABHD6, ABI2, ABL2, ACACA, ACAD8, |
| SLE vs. OA | ACAD9, ACADVL, ACAP3, ACAT2, ACER3, ACKR3, ACLY, ACO1, |
| | ACOX3, ACP1, ACRC, ACSL3, ACSL4, ACTA2, ACTG2, ACTL6A, |
| | ACTR10, ACTR1A, ACTR3B, ACTRT3, ACVR1, ADAM12, ADAM1A, |
| | ADAMTS1, ADAMTS12, ADAMTS15, ADAMTS16, ADAMTS2, |
| | ADAMTS3, ADAMTS6, ADAMTS9, ADAMTSL1, ADARB1, ADAT1, |
| | ADAT2, ADCY1, ADCY2, ADCY3, ADCY4, ADD1, ADGRA3, ADGRL1, |
| | ADGRL2, ADGRL4, ADIPOR2, ADNP, ADO, ADRA2A, AEBP1, AEN, |
| | AFF4, AFG3L1P, AFG3L2, AGAP1, AGAP3, AGBL5, AGFG1, AGL, |
| | AGMAT, AGPAT1, AGPAT4, AGPAT4-IT1, AGPAT6, AGT, AHCYL1, |
| | AHCYL2, AHI1, AHNAK2, AHSA2, AIFM1, AIG1, AIMP1, AIMP2, AK1, |
| | AK3, AK6, AKAP17A, AKAP8, AKIP1, AKR1C1, AKR1C2, AKR1C3, |
| | AKR1E2, AKR7A2, AKR7A3, AKT2, AKT3, ALAD, ALDH18A1, |
| | ALDH1A3, ALDH7A1, ALG13, ALG2, ALG6, ALG8, ALG9, ALKBH2, |
| | ALKBH3, ALMS1, ALS2, AMD1, AMFR, AMMECR1, AMMECR1L, |
| | AMOTL1, AMOTL2, AMPD2, AMT, AMZ2, AMZ2P1, ANAPC16, |
| | ANAPC4, ANAPC5, ANAPC7, ANGEL2, ANGPTL2, ANGPTL4, ANK3, |
| | ANKH, ANKHD1, ANKLE2, ANKRD10, ANKRD10-IT1, ANKRD13B, |
| | ANKRD13C, ANKRD28, ANKRD29, ANKRD35, ANKRD50, ANKRD54, |
| | ANKRD9, ANKS3, ANKS6, ANKZF1, ANO1, ANO10, ANO5, ANTXR1, |
| | ANXA11, AOC2, AOX1, AP3D1, AP3M1, AP3M2, AP4B1, AP4E1, |
| | APBB2, APEX2, APITD1, APLNR, APLP2, APOLD1, APP, APTR, APTX, |
| | AQP1, AQR, AR, ARCN1, ARF4, ARFGAP1, ARG2, ARHGAP21, |
| | ARHGAP28, ARHGAP29, ARHGAP5, ARHGEF10, ARHGEF12, |
| | ARHGEF17, ARHGEF37, ARHGEF9, ARID1B, ARIH2, ARL1, ARL10, |
| | ARL15, ARL3, ARL5B, ARL6, ARMC10, ARMC8, ARMC9, ARMCX1, |
| | ARMCX2, ARMCX3, ARMCX4, ARMCX5, ARMCX5-GPRASP2, |
| | ARMCX6, ARPC1A, ARPC5L, ARRB1, ARSD, ASAP1-IT2, ASAP3, |
| | ASB1, ASB6, ASB8, ASNS, ASNSD1, ASPN, ASS1, ASXL1, ATE1, ATF4, |
| | ATF7IP, ATF7IP2, ATG101, ATG12, ATG2A, ATG2B, ATG4B, ATIC, |
| | ATL2, ATL3, ATM, ATP11B, ATP13A3, ATP1A1, ATP2A2, ATP2B4, |
| | ATP5A1, ATP5G1, ATP5I, ATP5J, ATP5O, ATP6AP1, ATP6V0A2, |
| | ATP6V0E1, ATP6V0E2, ATP6V1C1, ATP8B1, ATP9A, ATPAF1, ATPIF1, |
| | ATR, ATRN, ATRX, ATXN1, ATXN10, ATXN2, AUTS2, AVEN, AVIL, |
| | AZI2, B3GALNT1, B3GALNT2, B3GALT6, B3GALTL, B3GAT3, |
| | B4GALT1, B4GALT2, B4GALT4, B4GALT6, B4GALT7, B4GAT1, |
| | BABAM1, BACE1, BACE2, BAD, BAG1, BAG2, BAMBI, BANK1, BAP1, |
| | BATF3, BAZ1B, BBS9, BCAR1, BCAR3, BCCIP, BCL2L2, BCL3, BCL6, |
| | BCOR, BCR, BDH2, BDP1, BECN1, BEGAIN, BEND6, BEND7, BET1L, |
| | BFAR, BGN, BHLHB9, BICC1, BICD1, BICD2, BIN3, BIN3-IT1, BIRC2, |
| | BIVM, BLCAP, BLMH, BLOC1S6, BMP1, BMP4, BMP8A, BMP8B, |
| | BMPR1A, BMPR2, BMS1, BMS1P20, BNC2, BNIP1, BOC, BOK, BOLA3- |
| | AS1, BPGM, BPHL, BRD1, BRD4, BRD8, BRD9, BRE, BRIX1, BRMS1L, |
| | BRPF3, BRWD1, BTBD1, BTBD11, BTBD3, BTBD7, BTRC, BUD31, |
| | BVES, BYSL, BZW1, BZW2, C10orf2, C10orf88, C11orf30, C11orf31, |
| | C11orf49, C11orf57, C11orf73, C11orf95, C12orf29, C12orf43, C12orf57, |
| | C12orf73, C14orf132, C14orf166, C14orf2, C14orf37, C15orf61, C16orf95, |
| | C17orf104, C17orf70, C17orf85, C17orf89, C19orf24, C19orf60, C1GALT1, |
| | C1orf109, C1orf122, C1orf216, C1orf43, C1orf50, C1orf52, C1orf53, |
| | C1orf56, C1QBP, C1R, C1RL, C20orf194, C21orf2, C21orf33, C22orf39, |
| | C2CD2, C2orf42, C2orf68, C2orf69, C2orf76, C4orf27, C4orf47, C5orf15, |
| | C5orf24, C5orf28, C5orf34, C5orf42, C5orf63, C6orf132, C6orf136, |
| | C6orf203, C6orf48, C7orf49, C8orf46, C8orf58, C8orf76, C9orf142, C9orf3, |
| | C9orf40, C9orf41, CA5BP1, CABLES1, CACNA1C, CACNA1G, |
| | CACNA2D1, CAD, CADM1, CADPS2, CALD1, CALU, CAMK2G, |
| | CAMK2N1, CAMKK2, CAMLG, CAMSAP2, CAMTA1, CAMTA2, |
| | CAND1, CAND2, CAP1, CAPN15, CAPN3, CAPN7, CAPRIN2, CARD14, |
| | CARKD, CARS, CARS2, CASC3, CASC4, CASD1, CASKIN2, CASP4, |
| | CBR4, CBX1, CBX3, CBX6, CBY1, CCAR1, CCAR2, CCDC107, |
| | CCDC113, CCDC130, CCDC137, CCDC14, CCDC142, CCDC149, |
| | CCDC176, CCDC25, CCDC3, CCDC66, CCDC8, CCDC80, CCDC84, |
| | CCDC97, CCHCR1, CCNB1IP1, CCND1, CCND2, CCNI, CCNJ, CCNL2, |
| | CCNT2, CCNY, CCS, CCSER2, CCT2, CCT3, CCT4, CCT5, CCT6A, |
| | CCT7, CCT8, CD163L1, CD276, CD2BP2, CD55, CD59, CD63, CD81, |
| | CD82, CD93, CD99L2, CDADC1, CDC123, CDC16, CDC23, CDC25B, |
| | CDC37, CDC37L1, CDC42BPA, CDCA7L, CDH11, CDH13, CDH23, |
| | CDH5, CDH6, CDIP1, CDK10, CDK14, CDK5RAP2, CDK5RAP3, CDK8, |
| | CDKN1C, CDKN2B, CDON, CEBPZOS, CELF1, CELF6, CEMIP, |
| | CENPBD1, CENPN, CEP112, CEP162, CEP164, CEP192, CEP290, CEP41, |
| | CEP44, CEP63, CEP70, CEP78, CEP83, CEP95, CERK, CERS2, CERS4, |
| | CFAP20, CFAP36, CFAP44, CFAP69, CFAP99, CFDP1, CFI, CHCHD1, |
| | CHCHD4, CHD1L, CHD3, CHD4, CHD6, CHD7, CHD9, CHEK2, CHERP, |
| | CHIC1, CHKA, CHKB, CHMP1A, CHMP7, CHN1, CHORDC1, CHST3, |
| | CHSY1, CHSY3, CHTOP, CIAO1, CIAPIN1, CILP, CIRBP, CISD1, |
| | CKMT2-AS1, CLASP1, CLASRP, CLCC1, CLCN4, CLCN5, CLCN6, |
| | CLDN12, CLEC11A, CLIC6, CLIP1, CLIP2, CLK4, CLMP, CLN8, CLOCK, |
| | CLSTN1, CLSTN2, CLTC, CLU, CLUAP1, CMBL, CMC2, CMC4, CMIP, |
| | CMSS1, CMTM4, CMTR2, CNIH1, CNKSR3, CNNM2, CNOT2, CNOT4, |
| | CNPY4, CNRIP1, CNTLN, COA3, COA5, COASY, COBL, COG3, COG4, |
| | COG7, COG8, COL11A1, COL12A1, COL13A1, COL14A1, COL15A1, |
| | COL16A1, COL18A1, COL1A1, COL1A2, COL21A1, COL22A1, |
| | COL27A1, COL3A1, COL4A1, COL5A1, COL5A2, COL5A3, COL6A2, |
| | COL6A3, COL8A1, COL8A2, COMMD2, COMMD4, COMMD6, COMP, |
| | COPRS, COPS3, COPS4, COPS5, COPS6, COPS7B, COPS8, COPZ2, |
| | COQ10A, COQ3, COQ4, COQ5, COQ6, CORO2B, COX14, COX18, |
| | COX20, COX4I1, COX7A1, COX7C, CPD, CPNE1, CPNE3, CPQ, CPSF3, |
| | CPSF3L, CPSF6, CPT2, CPXM2, CRAMP1L, CRAT, CREB5, CREBBP, |
| | CREBZF, CRELD1, CRELD2, CREM, CRISPLD2, CRK, CRLF1, CRNDE, |
| | CROCCP2, CROT, CRTAC1, CRTAP, CRTC3, CRY1, CRYBG3, CRYZL1, |
| | CSAD, CSDE1, CSE1L, CSGALNACT1, CSNK1A1, CSNK1D, CSNK1E, |
| | CSNK1G2, CSNK2A2, CSPG4, CSRNP2, CSRNP3, CSTF3, CTAGE5, |
| | CTBP1, CTC1, CTDSP1, CTDSP2, CTDSPL, CTNNAL1, CTPS1, CTPS2, |
| | CTSF, CTSK, CTTN, CUL4A, CUL5, CUL9, CUX1, CXorf56, CXorf57, |
| | CYB561A3, CYB5D2, CYB5R1, CYB5RL, CYCS, CYHR1, CYP27C1, |
| | CYP2R1, CYP4V2, CYP4X1, CYTH3, CYYR1, DAAM1, DAB2, DAB2IP, |
| | DAG1, DALRD3, DANCR, DAP, DAP3, DARS, DAW1, DAZAP1, DBN1, |
| | DBT, DCAF10, DCAF13, DCAF16, DCAF5, DCAF8, DCBLD2, DCLRE1C, |
| | DCN, DCP1B, DCTD, DCTN2, DCUN1D4, DCUN1D5, DCXR, DDAH1, |
| | DDHD2, DDIT3, DDT, DDX1, DDX10, DDX19A, DDX20, DDX21, |
| | DDX26B, DDX27, DDX3X, DDX42, DDX47, DDX49, DDX5, DDX50, |
| | DDX51, DDX52, DDX54, DDX55, DDX56, DECR2, DEFB124, DEPDC5, |
| | DEPDC7, DESI2, DEXI, DFFA, DFNB31, DFNB59, DGCR2, DGKD, |
| | DGKE, DGUOK, DHFR, DHFRL1, DHTKD1, DHX15, DHX16, DHX29, |
| | DHX30, DHX32, DHX33, DHX37, DHX38, DHX57, DHX8, DIO2, DIP2A, |
| | DIP2C, DIRAS3, DIS3, DIS3L, DISP1, DIXDC1, DKC1, DKK3, DLC1, |
| | DLG3, DLG5, DLGAP4, DLL1, DLX4, DMAP1, DMD, DMPK, DMTF1, |
| | DNAH1, DNAJA3, DNAJA4, DNAJB1, DNAJB11, DNAJB2, DNAJB4, |
| | DNAJC10, DNAJC12, DNAJC15, DNAJC16, DNAJC2, DNAJC21, |
| | DNAJC25, DNAJC27, DNAJC30, DNAJC4, DNAJC8, DNAL1, DNAL4, |
| | DNALI1, DNM1, DNM1L, DNM3OS, DNMBP, DNMT3B, DOCK6, DOHH, |
| | DOK4, DOK5, DONSON, DOPEY1, DPAGT1, DPCD, DPH5, DPM2, DPP7, |
| | DPP8, DPT, DPY19L1, DPY19L3, DPY19L4, DPYSL3, DPYSL4, |
| | DROSHA, DSEL, DSPP, DST, DSTN, DSTYK, DTD2, DTWD1, DTX3, |
| | DTYMK, DUSP14, DUSP18, DUSP22, DUT, DVL1, DVL3, DYM, |
| | DYNC1LI2, DYNC2H1, DYNC2LI1, DYNLL1, DYNLL2, DZIP1, DZIP3, |
| | E2F6, EARS2, EBF1, EBF2, EBLN2, EBLN3, EBPL, ECHDC2, ECHS1, |
| | ECM2, ECSCR, ECSIT, EDEM3, EDF1, EDIL3, EDRF1, EEA1, EEF1A1, |
| | EEF1D, EEF1E1, EEF2, EFEMP1, EFEMP2, EFHC1, EFNA5, EFNB1, |
| | EFNB2, EFS, EGFL7, EHBP1, EHD2, EHD4, EI24, EID2, EIF1, EIF1AX, |
| | EIF2AK4, EIF2B1, EIF2B3, EIF2B4, EIF2B5, EIF2D, EIF2S2, EIF2S3, |
| | EIF3A, EIF3B, EIF3D, EIF3E, EIF3F, EIF3H, EIF3I, EIF3J, EIF3K, EIF3L, |
| | EIF3M, EIF4B, EIF4E2, EIF4EBP2, EIF4G2, EIF4G3, EIF4H, EIF5, EIF5A2, |
| | EIF5B, EIF6, ELAC1, ELAC2, ELK1, ELL2, ELL3, ELMOD2, ELN, |
| | ELOVL1, ELOVL4, ELOVL6, ELP2, ELP3, ELP5, ELP6, EMC1, EMC3, |
| | EMC3-AS1, EMC9, EMG1, EMP1, EMP2, EMX2, EMX2OS, ENAH, |
| | ENDOD1, ENDOG, ENGASE, ENO2, ENOX1, ENOX2, ENPEP, ENPP1, |
| | ENPP2, ENPP5, ENPP6, ENTPD1, ENTPD1-AS1, ENTPD6, EOGT, EP400, |
| | EPAS1, EPB41L4A, EPB41L5, EPC1, EPDR1, EPG5, EPHA3, EPHA4, |
| | EPHB4, EPM2A, EPM2AIP1, EPN2, EPS8L2, EPYC, ERBB2, ERBB2IP, |
| | ERCC1, ERCC3, ERCC5, ERCC6L2, ERGIC2, ERLIN2, ERMARD, |
| | ERMP1, ERVK13-1, ESD, ESF1, ESYT2, ETAA1, ETF1, ETFB, ETFDH, |
| | EVA1C, EVC, EXO5, EXOC2, EXOC6, EXOC6B, EXOSC6, EXOSC7, |
| | EXPH5, EXT1, EXT2, EXTL2, F5, FADS1, FAF1, FAHD1, FAHD2A, |
| | FAHD2CP, FAM110B, FAM110C, FAM114A1, FAM114A2, FAM118B, |
| | FAM120A, FAM120B, FAM120C, FAM126A, FAM126B, FAM134B, |
| | FAM134C, FAM149A, FAM160A2, FAM160B1, FAM160B2, FAM162A, |
| | FAM168A, FAM168B, FAM171A1, FAM171B, FAM172A, FAM173B, |
| | FAM178A, FAM193A, FAM193B, FAM19A5, FAM200B, FAM207A, |
| | FAM20B, FAM20C, FAM210B, FAM219B, FAM222B, FAM228B, |
| | FAM24B, FAM35A, FAM3A, FAM3C, FAM43A, FAM43B, FAM49A, |
| | FAM50A, FAM53C, FAM57A, FAM63B, FAM73B, FAM76B, FAM78B, |
| | FAM92A1, FAM98A, FAM98B, FAN1, FANCG, FANCL, FAP, FARP1, |
| | FARP2, FARS2, FARSB, FASTKD1, FASTKD2, FAT1, FAT4, FBL, |
| | FBLIM1, FBLN2, FBLN7, FBN1, FBRSL1, FBXL17, FBXL19, FBXL2, |
| | FBXL7, FBXL8, FBXO11, FBXO17, FBXO18, FBXO21, FBXO22, |
| | FBXO30, FBXO32, FBXO33, FBXO45, FBXO9, FBXW2, FCER1A, FCF1, |
| | FDPS, FEM1A, FER, FERMT2, FEZ1, FGD5, FGF13, FGF18, FGF2, |
| | FGFBP2, FGFR1, FGFR10P, FH, FHIT, FHL2, FHOD1, FIBP, FIGN, |
| | FILIP1, FIP1L1, FITM2, FKBP10, FKBP14, FKBP1B, FKBP4, FKBP7, |
| | FKBP9, FKRP, FKTN, FLJ32255, FLJ37035, FLJ37453, FLJ42627, FLRT2, |
| | FLT1, FLVCR1, FMNL3, FMOD, FNBP1L, FNBP4, FNDC4, FNIP2, |
| | FOCAD, FOSL2, FOXC1, FOXJ3, FOXN3, FOXO1, FOXP1, FOXP1-IT1, |
| | FOXP2, FRA10AC1, FRK, FRMD6, FRS2, FSCN1, FSD1L, FSTL1, FTSJ1, |
| | FTSJ2, FTSJ3, FUBP1, FUBP3, FUT11, FXN, FZD1, FZD10-AS1, FZD3, |
| | FZD8, GABBR1, GABPA, GABPB1-AS1, GABPB2, GABRB2, |
| | GADD45GIP1, GAK, GALNT10, GALNT11, GALNT18, GALNT2, |
| | GALNT5, GAN, GAP43, GAPLINC, GAPVD1, GAR1, GAREM, GAREML, |
| | GART, GAS5, GAS6, GAS6-AS1, GATA2, GATA6, GATAD1, GATAD2A, |
| | GATC, GATSL2, GCAT, GCDH, GCFC2, GCLM, GCN1L1, GDF5, |
| | GDPD5, GEMIN2, GEMIN4, GEMIN6, GEMIN8, GET4, GFPT2, GFRA1, |
| | GGA1, GGCT, GGCX, GGNBP2, GGPS1, GGT7, GID8, GJA1, GJA4, |
| | GJA5, GJB2, GJB6, GK5, GKAP1, GLB1L, GLI3, GLIDR, GLIS2, GLIS3, |
| | GLMN, GLRB, GLS, GLT8D1, GLT8D2, GLTSCR2, GMDS, GMPPA, |
| | GMPS, GNA11, GNAL, GNAQ, GNB2L1, GNG11, GNG12, GNL1, GNL2, |
| | GNL3, GNL3L, GNPAT, GNPNAT1, GOLGA1, GOLGA2, GOLGA2P10, |
| | GOLGA3, GOLGA4, GOLGA8A, GOLGA8N, GOLGB1, GOLM1, GOPC, |
| | GORAB, GORASP1, GOSR2, GOT2, GPAA1, GPALPP1, GPATCH2L, |
| | GPATCH4, GPATCH8, GPBP1, GPC4, GPD1L, GPER1, GPHN, GPM6B, |
| | GPN2, GPR107, GPR153, GPR161, GPR180, GPR88, GPRC5A, GPRC5C, |
| | GPSM1, GPX7, GPX8, GRAMD3, GRB10, GRHL1, GRHPR, GRIA3, |
| | GRK4, GRK5, GRSF1, GSPT1, GSPT2, GSS, GSTA4, GSTM3, GTF2A2, |
| | GTF2H4, GTF2H5, GTF3A, GTF3C1, GTF3C3, GTF3C5, GTPBP4, |
| | GUCA1A, GUCY1A2, GUCY1A3, GUCY1B3, GUF1, GYS1, GZF1, H19, |
| | H2AFV, H2BFXP, H6PD, HABP4, HACD3, HACE1, HADH, HADHA, |
| | HAGLR, HARS, HARS2, HAS1, HAS2, HAUS7, HCFC2, HDAC11, |
| | HDAC2, HDAC4, HDAC7, HDAC8, HDDC2, HDGFRP3, HDHD2, HDLBP, |
| | HEATR1, HEATR3, HEATR5A, HECTD2, HECTD4, HEG1, HELQ, HELZ, |
| | HEPH, HERC2, HERC4, HES1, HEY2, HEYL, HGF, HGSNAT, HHLA3, |
| | HIBCH, HIF1A, HIF1AN, HILPDA, HIP1, HIVEP2, HKR1, HLTF, |
| | HMBOX1, HMBS, HMG20A, HMGA2, HMGCS1, HMGN1, HMGN3, |
| | HNRNPA1, HNRNPA2B1, HNRNPA3, HNRNPAB, HNRNPH3, HNRNPM, |
| | HNRNPU, HNRNPU-AS1, HNRNPUL2, HOMER1, HOMER3, HOOK2, |
| | HOOK3, HOTAIRM1, HOTS, HOXA11, HOXA5, HOXB5, HOXB6, |
| | HOXB7, HOXC4, HOXC9, HOXD4, HOXD8, HP, HP1BP3, HPS4, HRAS, |
| | HRH1, HS3ST1, HSBP1, HSD17B1, HSDL1, HSDL2, HSF2, HSP90AA1, |
| | HSP90AB1, HSPA12A, HSPA12B, HSPA14, HSPA4L, HSPA5, HSPA8, |
| | HSPD1, HSPE1, HSPG2, HSPH1, HTR2A, HTRA1, HTRA2, HTT, HUWE1, |
| | HYAL2, HYI, IARS, IARS2, IBA57, IBTK, ICE1, ICMT, ID1, ID4, IDH2, |
| | IDH3B, IDI1, IER3IP1, IER5L, IFRD1, IFRD2, IFT122, IFT140, IFT20, |
| | IFT22, IFT43, IFT80, IGDCC4, IGF1, IGF2BP2, IGFBP2, IGFBP4, IGSF3, |
| | IKBIP, IKBKAP, IKBKB, IKZF4, IL11RA, IL13RA1, IL13RA2, IL17D, |
| | IL17RD, IL18R1, IL6, ILF2, ILF3, ILKAP, IMMP1L, IMMT, IMP3, |
| | IMPAD1, IMPDH2, INAFM1, ING4, ING5, INHBA, INMT, INPP1, |
| | INPP5A, INPP5B, INPP5F, INPPL1, INTS10, INTS4, INTS7, INTS8, INTU, |
| | IP6K1, IP6K2, IPO4, IPO5, IPO7, IPO8, IPO9, IPP, IPW, IQCE, IQSEC1, |
| | IRGQ, IRS1, ISG20L2, ISM1, IST1, ISY1, ITFG1, ITGA1, ITGA3, ITGA7, |
| | ITGAE, ITGAV, ITGB1, ITGB1BP1, ITGB5, ITIH4, ITPA, ITPR2, ITSN1, |
| | ITSN2, IVD, IWS1, JADE1, JAG1, JAM2, JAM3, JMJD4, JMJD6, JMJD8, |
| | JRK, JTB, KAL1, KALRN, KANK2, KANSL1L, KARS, KAT2A, |
| | KATNAL1, KAZN, KBTBD6, KCMF1, KCND3, KCNE4, KCNJ15, KCNJ5, |
| | KCNJ6, KCNK1, KCNK3, KCNK6, KCNN3, KCNQ10T1, KCNS3, KCNT2, |
| | KCTD1, KCTD10, KCTD15, KCTD2, KCTD3, KDELC1, KDELC2, |
| | KDELR2, KDELR3, KDM4B, KDM4C, KDM5B, KDSR, KGFLP2, |
| | KHDRBS1, KHNYN, KHSRP, KIAA0020, KIAA0141, KIAA0226L, |
| | KIAA0232, KIAA0368, KIAA0430, KIAA0753, KIAA0895, KIAA0895L, |
| | KIAA0930, KIAA1109, KIAA1217, KIAA1279, KIAA1324L, KIAA1429, |
| | KIAA1462, KIAA1644, KIAA1715, KIAA1841, KIF1B, KIF1C, KIF21A, |
| | KIF26A, KIF3B, KIF7, KIF9, KIFAP3, KIRREL, KIZ, KLC1, KLF7, |
| | KLHDC1, KLHDC10, KLHDC4, KLHL13, KLHL21, KLHL22, KLHL29, |
| | KLHL3, KLHL36, KLHL42, KLHL5, KLHL9, KMT2A, KNOP1, KPNA2, |
| | KPNA4, KPNA5, KPNA6, KPNB1, KRBOX4, KREMEN1, KRI1, KRIT1, |
| | KRR1, KRT10, KSR1, KTN1, L3MBTL1, L3MBTL3, LAMA2, LAMA4, |
| | LAMA5, LAMB1, LAMB2, LAMC1, LAMTOR5, LANCL1, LAPTM4A, |
| | LAPTM4B, LARGE, LARS, LATS2, LBH, LCA5, LCAT, LCLAT1, |
| | LCMT1, LCMT2, LDB2, LDHB, LDLR, LDLRAP1, LDOC1L, LENG8, |
| | LEO1, LETM1, LETMD1, LGALS8, LGALSL, LGI2, LIG3, LIG4, LIMCH1, |
| | LINC00116, LINC00260, LINC00312, LINC00342, LINC00476, |
| | LINC00597, LINC00622, LINC00632, LINC00667, LINC00674, |
| | LINC00685, LINC00938, LINC01000, LINC01004, LINC01089, |
| | LINC01116, LINC01128, LINC01137, LINC01272, LINC01279, |
| | LINC01355, LINC01420, LINC01503, LINS, LIX1L, LLPH, LMAN2L, |
| | LMBR1, LMF1, LMLN, LMTK2, LNX2, LOC100129034, LOC100129550, |
| | LOC100132352, LOC100133039, LOC100133315, LOC100272216, |
| | LOC100289058, LOC100289098, LOC100505498, LOC100505715, |
| | LOC100506476, LOC100506548, LOC100506730, LOC100507316, |
| | LOC101927151, LOC101927752, LOC101927811, LOC101928000, |
| | LOC101928524, LOC101928673, LOC101928762, LOC101929243, |
| | LOC102606465, LOC102723919, LOC102724814, LOC102724851, |
| | LOC102724927, LOC103344931, LOC145474, LOC149401, LOC153682, |
| | LOC154761, LOC155060, LOC157562, LOC200772, LOC284454, |
| | LOC286052, LOC286272, LOC286437, LOC338620, LOC388692, |
| | LOC389765, LOC441081, LOC642236, LOC642852, LOC646014, |
| | LOC646762, LOC728024, LOC728093, LOC728392, LOC729680, |
| | LOC93622, LONP1, LONRF3, LOX, LPAR1, LPAR4, LPCAT1, LPGAT1, |
| | LPIN1, LPP, LPPR2, LRBA, LRIG2, LRP1, LRP11, LRP12, LRP1B, LRP3, |
| | LRP6, LRPAP1, LRPPRC, LRRC15, LRRC32, LRRC37A2, LRRC37A3, |
| | LRRC47, LRRC58, LRRC59, LRRC69, LRRN4CL, LSG1, LSM10, LSM11, |
| | LSM7, LSM8, LTBP2, LTBP3, LTBR, LTC4S, LTV1, LUC7L, LUC7L2, |
| | LUC7L3, LXN, LYNX1, LYRM4, LYRM7, LZTR1, LZTS1, MACF1, |
| | MADD, MAEA, MAFG, MAGED1, MAGED2, MAGEE1, MAGI1, MAGI2- |
| | AS3, MAGI2-IT1, MAGI3, MAGT1, MAK16, MALL, MAML1, MAN1A2, |
| | MAN1B1, MAN1C1, MAN2A1, MAN2A2, MAN2B2, MANBAL, |
| | MANSC1, MAP1A, MAP1B, MAP1LC3A, MAP2, MAP2K2, MAP2K4, |
| | MAP2K5, MAP3K12, MAP3K3, MAP3K4, MAP3K7, MAP3K7CL, MAP4, |
| | MAP4K2, MAP4K4, MAP4K5, MAP7D3, MAP9, MAPK8, MAPK8IP3, |
| | MAPK9, MAPKAPK5-AS1, MAPRE2, MARK1, MARK3, MARVELD1, |
| | MAST2, MAST3, MAST4, MAT2A, MATN2, MATR3, MAU2, MAVS, |
| | MBD3, MBD5, MBOAT2, MBTPS1, MCAM, MCAT, MCC, MCCC2, |
| | MCF2L, MCM3AP, MCM7, MCM8, MCOLN1, MCOLN3, MCPH1, |
| | MCTS1, MDC1, MDH2, MDM1, MDM4, MDN1, ME3, MECOM, MECP2, |
| | MED13, MED14, MED21, MED23, MED24, MED27, MED28, MED29, |
| | MED8, MEDAG, MEG3, MEG9, MEGF8, MEIS1, MEIS2, MEIS3P1, |
| | MEOX2, MESP1, METAP1, METAP2, METRN, METTL1, METTL10, |
| | METTL13, METTL15, METTL16, METTL17, METTL21B, METTL22, |
| | METTL23, METTL2B, METTL3, METTL8, METTL9, MFAP2, MFAP3, |
| | MFAP3L, MFF, MFI2, MFN1, MFSD10, MFSD7, MGAT5, MGC24103, |
| | MGC27345, MGC57346, MGP, MIA3, MIB2, MICAL2, MICAL3, |
| | MICALL2, MICU3, MID1, MID2, MIEF1, MIEN1, MIIP, MINOS1, MIOS, |
| | MIPEP, MIR100HG, MIR143HG, MIR181A2HG, MIR22HG, MIR29C, |
| | MIR34A, MIR3682, MIR99AHG, MIRLET7BHG, MIRLET7D, MIS12, |
| | MKKS, MKLN1, MLF1, MLH1, MLH3, MLIP, MLLT1, MLLT11, MLLT3, |
| | MLLT4, MLLT6, MLPH, MLXIP, MMAA, MMGT1, MMRN2, MN1, |
| | MOCS1, MOCS2, MOK, MON1B, MORC2, MORC4, MORF4L2, MORN2, |
| | MOSPD1, MPDZ, MPHOSPH10, MPHOSPH8, MPHOSPH9, MPRIP, |
| | MPZL2, MRC2, MRGPRF, MRU, MRO, MRPL1, MRPL19, MRPL21, |
| | MRPL24, MRPL28, MRPL35, MRPL36, MRPL37, MRPL43, MRPL46, |
| | MRPL50, MRPL51, MRPL9, MRPS11, MRPS16, MRPS17, MRPS21, |
| | MRPS25, MRPS27, MRPS30, MRPS33, MRPS34, MRPS5, MRPS6, MRS2, |
| | MRVI1, MSH3, MSH6, MSL1, MSL3, MSRB2, MSRB3, MSTO1, MSX1, |
| | MSX2, MTA1, MTCH1, MTERF2, MTERF3, MTF1, MTFR1L, MTG2, |
| | MTHFD2L, MTMR11, MTMR2, MTMR4, MTMR9, MTMR9LP, MTOR, |
| | MTRF1, MTRF1L, MTURN, MTX2, MTX3, MUC1, MUC20, MUM1, |
| | MUTYH, MXRA5, MXRA7, MYEOV2, MYH10, MYH11, MYL6, MYO10, |
| | MYO15B, MYO1B, MYO1C, MYO6, MYO7A, MYO9A, MYOC, MZF1, |
| | MZT1, N4BP2L2, N6AMT2, NAA10, NAA16, NAA25, NAA38, NAA40, |
| | NAB1, NABP1, NACA, NACAP1, NACC2, NADSYN1, NAE1, NALCN, |
| | NAMPT, NAP1L1, NAP1L3, NAP1L4, NAP1L5, NAPEPLD, NARF, |
| | NAT10, NAT9, NAV1, NAV2, NBAS, NBEA, NBL1, NBPF1, NBPF3, |
| | NCALD, NCBP2, NCBP2-AS2, NCKAP1, NCKAP5, NCLN, NCOA1, |
| | NCOA5, NCOR2, NCR3LG1, NDC1, NDEL1, NDFIP2, NDN, NDNL2, |
| | NDP, NDUFA10, NDUFAF1, NDUFAF5, NDUFAF6, NDUFAF7, NDUFB2, |
| | NDUFS6, NDUFV1, NECAP1, NEDD4, NEDD4L, NEDD8, NEFH, NEIL2, |
| | NEK3, NEK6, NEK9, NELFCD, NEMF, NENF, NEO1, NET1, NEU3, |
| | NEURL4, NF1, NF2, NFATC1, NFATC2, NFATC2IP, NFATC3, NFATC4, |
| | NFIB, NFIC, NFIL3, NFRKB, NFS1, NFYA, NFYB, NGEF, NGF, NGFR, |
| | NGFRAP1, NGRN, NHLRC2, NHP2, NHP2L1, NID1, NID2, NIFK, NINJ2, |
| | NINL, NISCH, NIT2, NKIRAS1, NKIRAS2, NKX3-2, NLGN4X, NLN, |
| | NLRP1, NMD3, NME3, NME6, NMNAT1, NMT2, NNMT, NNT-AS1, |
| | NOB1, NOC2L, NOC3L, NOD1, NOL10, NOL11, NOL3, NOL8, NOL9, |
| | NOLC1, NOM1, NOP10, NOP14, NOP16, NOP2, NOP58, NOTCH1, |
| | NOTCH4, NOV, NPAS2, NPDC1, NPFF, NPHP3, NPHP4, NPIPA1, |
| | NPLOC4, NPM1, NPM3, NPNT, NPR1, NPR2, NPR3, NPRL2, NPRL3, |
| | NR2F2, NR2F6, NRAV, NRBF2, NRBP2, NRD1, NRF1, NRK, NRN1, |
| | NRXN2, NSA2, NSDHL, NSMAF, NSMCE1, NSMCE4A, NSMF, NSUN4, |
| | NSUN5, NSUN5P1, NSUN6, NT5C3B, NT5DC2, NT5E, NTMT1, NTN1, |
| | NTNG1, NTRK2, NUAK1, NUBP2, NUDC, NUDCD1, NUDT11, NUDT15, |
| | NUDT16L1, NUDT3, NUDT5, NUDT9, NUFIP2, NUMB, NUP133, |
| | NUP155, NUP160, NUP188, NUP43, NUP54, NUP85, NUP98, NUPL1, |
| | NXF1, NXN, NXT1, OBSL1, OCIAD1, OCRL, ODC1, ODF2, ODF2L, |
| | OFD1, OGFOD1, OGFOD2, OGFOD3, OGG1, OGN, OIP5-AS1, |
| | OLFML2A, OLFML3, OPA1, ORAI2, ORAOV1, ORC5, OSBPL6, OSCP1, |
| | OSMR, OSTC, OTUD3, OTUD4, OTUD7B, OTULIN, OXCT1, OXR1, |
| | P3H1, P3H3, P3H4, P4HA2, P4HA3, P4HB, PABPC1L, PABPN1, PACRGL, |
| | PACS2, PAF1, PAFAH1B1, PAFAH1B2, PAICS, PAIP1, PALM2, PAM, |
| | PAN2, PANK4, PANX1, PAOX, PAPD7, PAPLN, PAPPA, PAQR3, PARD3, |
| | PARD3B, PARK7, PARN, PARP1, PARP6, PARVA, PARVB, PATZ1, |
| | PAWR, PAXBP1, PAXIP1, PBX1, PBXIP1, PC, PCAT19, PCCA, PCDH12, |
| | PCDH7, PCDH9, PCDHB10, PCDHB14, PCDHB15, PCDHB16, PCDHB2, |
| | PCDHB5, PCDHB6, PCED1A, PCF11, PCGF2, PCGF3, PCGF6, PCM1, |
| | PCMT1, PCNT, PCNX, PCNXL2, PCNXL4, PCOLCE, PCSK1, PCSK5, |
| | PCSK7, PCYT1A, PDCD11, PDCD6, PDDC1, PDE1A, PDE1C, PDE3A, |
| | PDE4B, PDE4D, PDE5A, PDE7B, PDE8A, PDGFD, PDGFRA, PDGFRB, |
| | PDGFRL, PDHA1, PDHB, PDK3, PDLIM4, PDLIM7, PDPK1, PDPN, |
| | PDPR, PDRG1, PDS5A, PDSS2, PDXK, PDZD8, PDZRN3, PDZRN4, |
| | PEAK1, PEAR1, PEBP1, PEG10, PENK, PET117, PEX1, PEX10, PEX11A, |
| | PFAS, PFKM, PFN2, PGAP1, PGAP3, PGBD1, PGBD2, PGM1, PGM3, |
| | PGR, PGRMC2, PHACTR4, PHAX, PHB, PHBP19, PHC1, PHF1, PHF10, |
| | PHF13, PHF14, PHF2, PHF20, PHF20L1, PHIP, PHKA2, PHKB, PHLDB1, |
| | PHLDB2, PHPT1, PHTF1, PHTF2, PHYH, PHYHIP, PHYKPL, PI15, |
| | PI4KA, PI4KB, PIAS3, PIAS4, PIBF1, PIEZO2, PIFO, PIGF, PIGG, PIGK, |
| | PIGL, PIGM, PIGT, PIGW, PIK3C2B, PIK3C3, PINK1, PIP4K2B, PISD, |
| | PITHD1, PITRM1, PITX1, PJA1, PKD2, PKDCC, PKI55, PKM, PKN3, |
| | PKNOX1, PKNOX2, PKP4, PLA2G12A, PLA2G2A, PLA2R1, PLAC9, |
| | PLAGL1, PLAU, PLCB4, PLCD4, PLCE1, PLEKHA1, PLEKHA4, |
| | PLEKHA5, PLEKHA8, PLEKHG2, PLEKHG4, PLEKHH2, PLIN3, PLIN5, |
| | PLOD2, PLRG1, PLS3, PLXDC1, PLXNA2, PLXNA4, PLXNC1, PM20D2, |
| | PMEPA1, PMPCA, PMS1, PMS2P5, PMVK, PNN, PNP, PNPO, PODN, |
| | PODXL, POFUT1, POFUT2, POGLUT1, POGZ, POLA1, POLD2, POLE4, |
| | POLR1C, POLR1E, POLR2B, POLR2F, POLR2G, POLR2H, POLR2L, |
| | POLR3A, POLR3B, POLR3E, POLR3H, POM121L9P, POMGNT1, POMT1, |
| | POMT2, POMZP3, PON2, POP5, POPDC2, POU3F3, POU6F1, PP12719, |
| | PP7080, PPAN, PPAP2A, PPAPDC1B, PPAPDC3, PPARA, PPAT, PPIC, |
| | PPID, PPIE, PPIEL, PPIL1, PPIL4, PPIP5K1, PPIP5K2, PPM1F, PPP1R14A, |
| | PPP1R14B, PPP1R16A, PPP1R21, PPP1R3B, PPP1R3E, PPP1R7, PPP2R2A, |
| | PPP2R2D, PPP2R5E, PPP3CA, PPP3CB, PPP4R2, PPP6C, PPP6R2, |
| | PPP6R3, PPRC1, PQLC2, PRADC1, PRB3, PRCC, PRDM10, PRDM11, |
| | PRDM15, PRDM16, PRDM2, PRDM5, PRDM6, PRDX2, PRDX6, PREB, |
| | PRELP, PREP, PREPL, PREX2, PRICKLE1, PRICKLE2, PRIMPOL, |
| | PRKAA1, PRKAB2, PRKAR1A, PRKAR2A, PRKCA, PRKD1, PRKD2, |
| | PRKDC, PRKRA, PRKXP1, PRMT2, PRMT5, PROS1, PROSER1, PRPF3, |
| | PRPF31, PRPF38A, PRPF4B, PRPF8, PRRC2B, PRRG1, PRRG3, PRRT3- |
| | AS1, PRRX1, PRRX2, PRSS23, PRSS35, PRSS53, PRTFDC1, PRTG, |
| | PRUNE, PRUNE2, PSD3, PSEN2, PSMA7, PSMB5, PSMB6, PSMC5, |
| | PSMD1, PSMD2, PSMD9, PSMG3, PSMG4, PSPC1, PSPH, PTBP2, PTCD3, |
| | PTCHD3P1, PTDSS2, PTEN, PTGFR, PTGFRN, PTGIS, PTH1R, PTK2, |
| | PTMA, PTOV1, PTPDC1, PTPN11, PTPN13, PTPN14, PTPN21, PTPRD, |
| | PTPRE, PTPRF, PTPRG, PTPRK, PTPRM, PTPRN2, PTPRS, PTRF, |
| | PTRH2, PTS, PURA, PURB, PUS1, PUS3, PUS7, PUSH, PWP1, PWWP2A, |
| | PXDN, QARS, QTRT1, R3HDM1, RAB11B-AS1, RAB11FIP3, RAB11FIP5, |
| | RABB, RAB21, RAB22A, RAB24, RAB27B, RAB28, RAB29, RAB31, |
| | RAB34, RAB36, RAB3GAP2, RAB40B, RAB40C, RABEP2, RABEPK, |
| | RABGAP1, RABGEF1, RABL6, RAD1, RAD17, RAD23B, RAD50, |
| | RAD51-AS1, RAE1, RAF1, RAI1, RAK, RALBP1, RALGAPA1, |
| | RALGAPA2, RAMP1, RAMP3, RAN, RANBP1, RANBP10, RANBP2, |
| | RANBP6, RAP1A, RAPGEF6, RARRES2, RARS, RASL12, RASSF8, |
| | RASSF8-AS1, RAVER2, RBAK, RBBP6, RBBP9, RBFOX2, RBM10, |
| | RBM12, RBM15, RBM17, RBM18, RBM26, RBM28, RBM33, RBM4, |
| | RBM48, RBM6, RBMS1, RBMS2, RBMX, RBMX2, RBPMS, RBPMS2, |
| | RBSN, RC3H1, RCAN1, RCAN2, RCBTB2, RCCD1, RCN1, RCN2, |
| | RCOR1, RDH11, RDH13, RECK, REEP3, RER1, REV3L, REXO2, RFNG, |
| | RFT1, RFXAP, RGL2, RGL3, RGS12, RGS16, RGS3, RGS4, RHBDD3, |
| | RHBDF1, RHOBTB3, RHOJ, RHOT2, RILPL1, RIMKLB, RIN2, RINT1, |
| | RIOK1, RITA1, RLIM, RMDN2, RNASEH1, RNASEH2C, RND3, RNF10, |
| | RNF144A, RNF145, RNF150, RNF168, RNF180, RNF20, RNF216, RNF217, |
| | RNF219, RNF24, RNF34, RNF7, RNMTL1, RNPS1, ROBO4, ROM1, |
| | ROR1, RPA1, RPAIN, RPARP-AS1, RPGRIP1L, RPIA, RPL10A, RPL10L, |
| | RPL11, RPL12, RPL14, RPL15, RPL18, RPL19, RPL22, RPL24, RPL27, |
| | RPL29, RPL3, RPL31, RPL32P3, RPL34, RPL35A, RPL36A, RPL37, |
| | RPL37A, RPL4, RPL5, RPL6, RPL7, RPL8, RPLP0, RPLP1, RPLP2, RPN2, |
| | RPP14, RPP25, RPRD1A, RPRD1B, RPS12, RPS15, RPS15A, RPS16, |
| | RPS17, RPS2, RPS21, RPS23, RPS24, RPS25, RPS28, RPS3, RPS4X, RPS6, |
| | RPS9, RPUSD3, RQCD1, RRAGA, RRM1, RRN3, RRNAD1, RRP12, |
| | RRP15, RRP1B, RRP36, RRP9, RRS1, RSAD1, RSBN1, RSL1D1, RSPH3, |
| | RSPO2, RSPRY1, RSU1, RTKN, RTN4RL1, RTTN, RUFY2, RUNX1-IT1, |
| | RUNX1T1, RUVBL1, RWDD2B, RWDD4, RXRB, RYK, S100PBP, |
| | SAAL1, SACS, SAE1, SAFB2, SALL2, SAMD4B, SAMD5, SAMD8, |
| | SAMM50, SAP30L, SAR1A, SAR1B, SARS, SASH1, SAT2, SATB2, |
| | SAYSD1, SBNO1, SBSPON, SCAF4, SCAP, SCARA3, SCD5, SCFD2, |
| | SCG2, SCIN, SCMH1, SCML1, SCN4B, SCO1, SCRG1, SCRIB, SCYL2, |
| | SCYL3, SDC4, SDCCAG8, SDF2, SDHAF2, SDK1, SDK2, SDR39U1, |
| | SEC11A, SEC14L1P1, SEC16A, SEC16B, SEC22A, SEC23A, SEC23IP, |
| | SEC24C, SEC24D, SEC31A, SEC62, SEC63, SECISBP2, SEH1L, |
| | SELENBP1, SELO, SELP, SEMA3A, SEMA3D, SEMA3E, SEMA4C, |
| | SEMA4F, SEMA6D, SENP1, SENP6, SENP7, SEPHS1, SEPT4, SEPT6, |
| | SEPT7P2, SEPT8, SEPT9, SEPW1, SERPINB6, SERPINF1, SERPINH1, |
| | SERTAD2, SERTAD4, SERTAD4-AS1, SETD2, SETD4, SETD5, SETD7, |
| | SETMAR, SF1, SF3B2, SF3B5, SFI1, SFPQ, SFRP1, SFRP2, SFSWAP, |
| | SFXN1, SFXN5, SGCB, SGCD, SGCE, SGSM2, SH2B1, SH3BP4, |
| | SH3GLB1, SH3PXD2A, SH3PXD2B, SH3RF1, SH3RF3, SH3YL1, SHB, |
| | SHC1, SHC3, SHMT2, SHOX2, SHQ1, SHROOM1, SIAE, SIGMAR1, |
| | SIK2, SIK3, SIN3B, SIPA1L2, SIX1, SIX2, SIX3, SIX3-AS1, SIX4, SIX5, |
| | SKP2, SLC10A3, SLC12A2, SLC12A4, SLC16A1, SLC16A10, SLC16A2, |
| | SLC16A7, SLC17A7, SLC19A2, SLC20A1, SLC20A2, SLC22A1, |
| | SLC22A23, SLC22A3, SLC23A2, SLC25A1, SLC25A12, SLC25A13, |
| | SLC25A14, SLC25A17, SLC25A27, SLC25A3, SLC25A33, SLC25A38, |
| | SLC25A44, SLC25A46, SLC25A6, SLC26A2, SLC2A10, SLC2A12, |
| | SLC2A8, SLC30A4, SLC30A5, SLC30A6, SLC33A1, SLC35A1, SLC35A3, |
| | SLC35B2, SLC35B3, SLC35B4, SLC35C2, SLC35D1, SLC35E1, SLC35G1, |
| | SLC35G2, SLC37A3, SLC38A10, SLC39A10, SLC39A3, SLC39A4, |
| | SLC39A6, SLC41A1, SLC41A3, SLC44A2, SLC45A1, SLC47A1, |
| | SLC48A1, SLC5A3, SLC5A6, SLC6A2, SLC6A8, SLC7A1, SLC7A6, |
| | SLC9A9, SLC9B2, SLCO2A1, SLFN11, SLIT3, SLK, SLMAP, SLMO1, |
| | SLTM, SLU7, SMAD2, SMAD3, SMAD4, SMAD6, SMAD9, SMARCA1, |
| | SMARCA2, SMARCA4, SMARCC1, SMARCD2, SMARCD3, SMARCE1, |
| | SMC1A, SMEK2, SMG5, SMG9, SMIM10L1, SMIM11, SMIM13, SMIM7, |
| | SMO, SMOC1, SMOC2, SMPD1, SMTN, SMU1, SMURF1, SMURF2, |
| | SNAPC1, SNAPC4, SNAPC5, SNCAIP, SND1, SND1-IT1, SNED1, |
| | SNHG11, SNHG15, SNHG16, SNHG19, SNHG21, SNHG3, SNHG8, |
| | SNHG9, SNORA25, SNORD104, SNORD114-3, SNRK, SNRNP200, |
| | SNRNP70, SNRPA1, SNRPD1, SNTB2, SNX19, SNX21, SNX29, SNX33, |
| | SOBP, SOCS4, SOCS5, SOCS7, SOGA1, SON, SORBS2, SOWAHC, |
| | SOX12, SOX4, SOX8, SOX9, SP3, SPAG1, SPAG16, SPAG8, SPAG9, |
| | SPARC, SPATA18, SPATA20, SPATA5, SPATS2L, SPECC1, SPECC1L, |
| | SPG7, SPIDR, SPIN4, SPOCD1, SPOCK1, SPON1, SPON2, SPOP, |
| | SPRED2, SPRY4, SPRY4-IT1, SPSB3, SPTAN1, SPTSSA, SRD5A3, |
| | SREBF1, SREK1, SRF, SRGAP1, SRM, SRPRB, SRPX, SRR, SRRM2, |
| | SRRT, SRSF1, SRSF10, SRSF11, SRSF4, SRSF6, SRSF8, SS18L1, SSBP3, |
| | SSPN, SSRP1, SSU72, ST13, ST3GAL1, ST3GAL3, ST3GAL4, ST5, |
| | ST6GALNAC5, ST6GALNAC6, ST8SIA1, STAB1, STAG1, STARD13, |
| | STARD9, STAT3, STAT6, STAU1, STEAP1, STEAP2, STEAP4, STK11, |
| | STK11IP, STK19, STK25, STK3, STK36, STMN2, STMN3, STOML1, |
| | STON1, STOX2, STRBP, STS, STUB1, STX18, STX2, STXBP1, STXBP5, |
| | SUCLA2, SUCO, SUDS3, SUFU, SUGP2, SUGT1, SULF1, SULF2, SUMF1, |
| | SUN1, SUN2, SUPT20H, SUPT7L, SURF4, SURF6, SUSD2, SUSD4, |
| | SUSD5, SUV420H1, SVEP1, SWAP70, SWI5, SYMPK, SYNC, SYNE1, |
| | SYNGR1, SYNJ2, SYNPO, SYT17, SYTL2, SYTL4, SZT2, TACC2, TAF11, |
| | TAF13, TAF1C, TAF1D, TAF3, TAF9B, TAGLN, TAL1, TANC1, TAPT1- |
| | AS1, TARBP1, TARDBP, TARS, TARSL2, TAS2R14, TAX1BP1, TBC1D1, |
| | TBC1D13, TBC1D16, TBC1D17, TBC1D20, TBC1D22B, TBC1D24, |
| | TBC1D2B, TBC1D8B, TBCCD1, TBCD, TBCK, TBL1X, TBPL1, TBRG1, |
| | TBRG4, TBX15, TBX2, TBX3, TBX5, TCAF1, TCAIM, TCEAL2, TCEAL4, |
| | TCEAL7, TCEANC, TCEB2, TCERG1, TCF3, TCFL5, TCHH, TCHP, |
| | TCP1, TCTN1, TCTN3, TDG, TDP1, TDRD10, TDRP, TEAD1, TEAD2, |
| | TEAD3, TECPR1, TECPR2, TELO2, TENM1, TENM3, TEP1, TERF1, |
| | TERF2IP, TEX10, TEX261, TFB2M, TFCP2, TFCP2L1, TFDP2, TGFB3, |
| | TGFBR1, TGM2, THAP5, THAP9-AS1, THBD, THBS1, THBS2, THBS3, |
| | THBS4, THEM4, THG1L, THOC1, THOC2, THOC5, THOC7, THRA, |
| | THRB, THSD7A, THUMPD2, THY1, THYN1, TIA1, TIAL1, TIAM2, |
| | TIMM10B, TIMM13, TIMM17A, TIMM21, TIMM22, TIMM44, TIMM50, |
| | TIMM9, TIMMDC1, TIMP1, TIMP2, TJAP1, TK2, TLCD2, TLE2, TLN2, |
| | TM2D1, TM2D2, TM2D3, TM7SF3, TM9SF1, TMA16, TMCC1, TMCO1, |
| | TMCO3, TMCO6, TMED10, TMED3, TMEM106B, TMEM110, TMEM117, |
| | TMEM119, TMEM120A, TMEM120B, TMEM129, TMEM134, TMEM136, |
| | TMEM138, TMEM147, TMEM14A, TMEM150C, TMEM161A, |
| | TMEM161B-AS1, TMEM167A, TMEM168, TMEM17, TMEM182, |
| | TMEM185A, TMEM185B, TMEM186, TMEM196, TMEM200A, |
| | TMEM200B, TMEM216, TMEM218, TMEM223, TMEM231, TMEM237, |
| | TMEM242, TMEM248, TMEM255B, TMEM259, TMEM260, TMEM261, |
| | TMEM263, TMEM39A, TMEM41A, TMEM42, TMEM43, TMEM45A, |
| | TMEM47, TMEM5, TMEM50B, TMEM55A, TMEM57, TMEM63A, |
| | TMEM64, TMEM67, TMEM87A, TMEM87B, TMEM9, TMEM97, |
| | TMEM98, TMEM99, TMOD1, TMTC1, TMTC3, TMTC4, TMUB2, TMX2, |
| | TMX3, TNC, TNFAIP1, TNFRSF11B, TNFRSF19, TNFRSF25, TNKS, |
| | TNPO2, TNRC6A, TNRC6C, TOB2, TOM1L1, TOM1L2, TOMM20, |
| | TOMM5, TOMM6, TOMM7, TOP1MT, TOR1AIP2, TP53BP1, TP53BP2, |
| | TP53INP2, TP53RK, TP53TG1, TP73-AS1, TPBG, TPGS2, TPM1, TPM2, |
| | TPMT, TPST1, TPT1, TPT1-AS1, TRABD2B, TRAF5, TRAF7, TRAK2, |
| | TRAM1L1, TRAP1, TRAPPC11, TRAPPC12, TRAPPC3, TRAPPC6B, |
| | TRIAP1, TRIB2, TRIM2, TRIM27, TRIM32, TRIM37, TRIM4, TRIM41, |
| | TRIM44, TRIM56, TRIM58, TRIM61, TRIM65, TRIM68, TRIM8, TRIP11, |
| | TRIP12, TRMT10B, TRMT10C, TRMT13, TRMT2B, TRMT5, TRMT6, |
| | TRNT1, TRO, TRPC1, TRPM7, TRRAP, TSC1, TSC22D1, TSC22D2, |
| | TSEN2, TSHZ2, TSHZ3, TSPAN10, TSPAN11, TSPAN12, TSPAN18, |
| | TSPAN3, TSPAN6, TSPAN9, TSPYL2, TSR1, TST, TSTD2, TTBK2, |
| | TTC13, TTC14, TTC17, TTC19, TTC23, TTC26, TTC28, TTC3, TTC31, |
| | TTC38, TTC4, TTC8, TTI1, TTL, TTLL11, TTLL3, TTN-AS1, TTPAL, |
| | TUB, TUBB2A, TUBB2B, TUBB3, TUBE1, TUBG2, TUBGCP5, |
| | TUBGCP6, TUG1, TULP3, TUSC2, TUSC3, TWIST1, TWISTNB, TWSG1, |
| | TXLNA, TXN2, TXNDC15, TXNL4A, TXNRD2, TXNRD3, TYSND1, |
| | UACA, UBA5, UBA52, UBAP2, UBE2E3, UBE2G2, UBE2I, UBE2J2, |
| | UBE2L3, UBE2O, UBE2Q1, UBE2R2, UBE2V2, UBE4A, UBE4B, UBFD1, |
| | UBL4A, UBLCP1, UBP1, UBQLN4, UBR3, UBR4, UBR5, UBTF, |
| | UBXN2A, UBXN8, UCKL1, UFC1, UGGT1, UGGT2, UGP2, UHRF1BP1L, |
| | UHRF2, ULK2, UMPS, UNC119B, UNC13B, UNC45A, UNC5B, UNK, |
| | UPF1, UPF2, UPF3B, UQCC1, UQCR10, UQCRB, UQCRFS1, UQCRQ, |
| | URB1, URGCP, URI1, UROD, USB1, USP11, USP13, USP14, USP20, |
| | USP22, USP24, USP28, USP31, USP32, USP34, USP36, USP39, USP40, |
| | USP45, USP46, USP47, USP48, USP51, USP54, USPL1, UTP14A, UTP15, |
| | VAMP4, VANGL1, VAPA, VAPB, VARS, VAV2, VBP1, VCAN, VCL, |
| | VCPKMT, VDAC3, VEGFB, VEZF1, VGLL3, VGLL4, VKORC1, |
| | VKORC1L1, VMA21, VPRBP, VPS13A, VPS13D, VPS16, VPS26B, |
| | VPS33B, VPS35, VPS36, VPS37B, VPS45, VPS51, VPS53, VPS54, VSTM4, |
| | VWA1, VWF, WAC-AS1, WASF1, WBP1L, WBP4, WBSCR16, WBSCR22, |
| | WDFY3-AS2, WDPCP, WDR12, WDR13, WDR19, WDR27, WDR35, |
| | WDR4, WDR45, WDR46, WDR5, WDR53, WDR55, WDR59, WDR6, |
| | WDR60, WDR75, WDR77, WDR83OS, WDR90, WDR92, WDYHV1, |
| | WFS1, WHSC1, WIPF2, WISP1, WLS, WNT5B, WRN, WWTR1, XBP1, |
| | XG, XIAP, XIST, χPA, XPNPEP1, XPNPEP3, XPO5, XPOT, XPR1, |
| | XRCC5, XRCC6, XXYLT1, XYLT2, YAP1, YARS, YBEY, YES1, YIF1A, |
| | YIPF4, YIPF5, YIPF6, YKT6, YLPM1, YTHDC2, YTHDF1, YTHDF2, YY1, |
| | ZAK, ZBED3, ZBED5, ZBTB10, ZBTB11, ZBTB20, ZBTB21, ZBTB37, |
| | ZBTB41, ZBTB44, ZBTB46, ZBTB47, ZBTB48, ZBTB7A, ZBTB7C, |
| | ZC3H14, ZC3H15, ZC3H8, ZC3HAV1L, ZC3HC1, ZCCHC14, ZCCHC24, |
| | ZCCHC9, ZDHHC1, ZDHHC14, ZDHHC15, ZDHHC16, ZDHHC3, |
| | ZDHHC4, ZFAND2B, ZFAS1, ZFHX3, ZFHX4, ZFP1, ZFP30, ZFP37, |
| | ZFP62, ZFP82, ZFP90, ZFYVE21, ZFYVE27, ZHX1, ZHX2, ZHX3, ZIC1, |
| | ZKSCAN1, ZKSCAN5, ZKSCAN7, ZMAT2, ZMAT3, ZMIZ1, ZMIZ2, |
| | ZMYM1, ZMYM3, ZMYM5, ZNF10, ZNF106, ZNF112, ZNF117, ZNF12, |
| | ZNF121, ZNF131, ZNF133, ZNF135, ZNF137P, ZNF140, ZNF141, ZNF146, |
| | ZNF17, ZNF175, ZNF18, ZNF180, ZNF197, ZNF2, ZNF202, ZNF204P, |
| | ZNF211, ZNF212, ZNF219, ZNF23, ZNF230, ZNF234, ZNF248, ZNF251, |
| | ZNF26, ZNF260, ZNF263, ZNF264, ZNF268, ZNF271P, ZNF275, ZNF276, |
| | ZNF284, ZNF286A, ZNF292, ZNF3, ZNF30, ZNF300, ZNF300P1, ZNF302, |
| | ZNF318, ZNF32, ZNF320, ZNF322, ZNF324, ZNF326, ZNF329, ZNF337, |
| | ZNF343, ZNF346, ZNF354A, ZNF365, ZNF37A, ZNF37BP, ZNF385D, |
| | ZNF398, ZNF404, ZNF410, ZNF417, ZNF431, ZNF432, ZNF436-AS1, |
| | ZNF439, ZNF44, ZNF449, ZNF451, ZNF462, ZNF471, ZNF473, ZNF485, |
| | ZNF500, ZNF502, ZNF506, ZNF507, ZNF512, ZNF514, ZNF518A, ZNF521, |
| | ZNF529, ZNF532, ZNF540, ZNF542P, ZNF546, ZNF551, ZNF555, ZNF558, |
| | ZNF559, ZNF565, ZNF566, ZNF568, ZNF569, ZNF573, ZNF584, ZNF585A, |
| | ZNF585B, ZNF587B, ZNF593, ZNF596, ZNF599, ZNF600, ZNF605, |
| | ZNF606, ZNF607, ZNF608, ZNF614, ZNF621, ZNF626, ZNF629, ZNF638, |
| | ZNF639, ZNF654, ZNF662, ZNF667, ZNF677, ZNF678, ZNF680, ZNF682, |
| | ZNF69, ZNF691, ZNF692, ZNF697, ZNF7, ZNF70, ZNF703, ZNF704, |
| | ZNF721, ZNF738, ZNF747, ZNF763, ZNF764, ZNF766, ZNF767P, ZNF770, |
| | ZNF781, ZNF783, ZNF785, ZNF788, ZNF789, ZNF790-AS1, ZNF791, |
| | ZNF793, ZNF805, ZNF814, ZNF827, ZNF83, ZNF830, ZNF84, ZNF846, |
| | ZNF850, ZNF862, ZNF883, ZNHIT1, ZNRF1, ZPR1, ZRANB1, ZRANB2, |
| | ZSCAN18, ZSCAN21, ZSCAN30, ZSCAN32, ZSWIM7, ZXDA, ZXDC, |
| | ZZZ3 |
| Up- | AAK1, AASDH, AATBC, ABTB1, ACAP1, ACE, ACOXL, ACSF2, ACSF3, |
| regulated in | ACSL5, ACTR2, ACTR3, ACVR2B, ADA, ADAM28, ADAM8, |
| Synovium | ADAMDEC1, ADAMTSL4, ADD1, ADGRE2, ADGRE5, ADNP2, |
| RA vs. OA | ADRBK2, ADRM1, AGO1, AGO2, AGO3, AGPAT3, AIM1, AIM2, AK2, |
| | AK9, AKAP13, AKAP8, AKIRIN1, AKIRIN2, AKNA, ALG5, ALYREF, |
| | AMICA1, AMPD3, ANKRD11, ANKRD12, ANKRD13A, ANKRD13D, |
| | ANKRD32, ANKRD36B, ANKRD36BP2, ANKRD44, ANKRD49, |
| | ANXA2R, AOAH, AP1G1, AP1S2, AP2A1, AP3D1, APOBEC3C, |
| | APOBEC3G, APOL1, APOL6, ARAP2, ARHGAP1, ARHGAP15, |
| | ARHGAP17, ARHGAP18, ARHGAP19, ARHGAP25, ARHGAP26, |
| | ARHGAP30, ARHGAP4, ARHGAP9, ARHGDIB, ARHGEF1, ARHGEF2, |
| | ARHGEF6, ARID1A, ARID2, ARID3A, ARID3B, ARID4B, ARID5A, |
| | ARIH1, ARL4C, ARL8A, ARPC5L, ASAP1, ASAP1-IT1, ASCL2, ASH1L, |
| | ASPHD2, ASXL1, ATAD3A, ATF5, ATF7, ATF7IP, ATG16L2, ATP2A3, |
| | ATP2B1, ATP8A1, ATXN7, AUH, AURKB, B2M, BATF, BAχ, BAZ1A, |
| | BAZ2A, BBIP1, BCL11A, BCL11B, BCL2, BCL2A1, BCL2L11, BEX2, |
| | BIN2, BIRC3, BISPR, BLNK, BLVRA, BMS1P20, BPTF, BRD2, BRD3, |
| | BRD4, BRD7, BRSK1, BSPRY, BTG2, BTK, BTLA, BTN2A2, BTN3A1, |
| | BTN3A2, BTN3A3, BUB3, BZRAP1-AS1, C10orf128, C10orf54, C11orf21, |
| | C11orf31, C11orf71, C12orf76, C15orf48, C16orf54, C18orf21, C19orf66, |
| | C1QA, C21orf91, C3orf38, C5orf56, CAMTA1, CANX, CARD11, CARD16, |
| | CARD8, CARD8-AS1, CASK, CASP10, CBFB, CBL, CBLB, CBLN3, |
| | CBX4, CCDC134, CCDC167, CCDC50, CCDC60, CCDC64, CCDC69, |
| | CCDC88C, CCL13, CCL18, CCL4, CCL5, CCL8, CCNA2, CCNC, CCND2, |
| | CCND3, CCNG2, CCR2, CCR5, CCR6, CCR7, CD19, CD2, CD200, CD24, |
| | CD247, CD27, CD274, CD37, CD38, CD3D, CD3E, CD3G, CD4, CD40, |
| | CD44, CD47, CD48, CD5, CD52, CD53, CD6, CD69, CD72, CD74, CD79A, |
| | CD79B, CD80, CD83, CD86, CD8A, CD8B, CD96, CD99P1, CDC42, |
| | CDC42EP3, CDC42-IT1, CDC42SE1, CDC42SE2, CDCA7, CDK12, |
| | CDKN1B, CDS2, CDV3, CDYL2, CEBPA-AS1, CECR1, CELF1, CENPM, |
| | CEP128, CEP19, CEP350, CEP57, CEP85L, CFLAR, CFP, CHAC2, CHD1, |
| | CHD2, CHD4, CHN2, CHST11, CIDEB, CIITA, CISH, CITED2, CKAP2, |
| | CKLF, CKS2, CLCN3, CLCN7, CLDND1, CLEC10A, CLEC1A, CLEC2D, |
| | CLMN, CLUHP3, CMIP, CMPK2, CNKSR1, CNOT3, CNOT6L, CNOT7, |
| | CNTRL, COCH, COMMD3, CORO1A, CORO1B, COX15, COX18, CP, |
| | CPM, CPNE5, CR1, CRACR2A, CREBL2, CRLF3, CRTAM, CRYGS, |
| | CSF1R, CSF2RB, CSK, CSNK1E, CSNK1G1, CSNK2A1, CST7, CSTF2T, |
| | CTSC, CTSH, CTSS, CTSW, CTSZ, CXCL1, CXCL10, CXCL11, CXCL13, |
| | CXCL2, CXCL3, CXCL9, CXCR3, CXCR4, CYB561, CYFIP2, CYLD, |
| | CYP27A1, CYP2S1, CYSLTR1, CYTIP, DAPP1, DAXX, DBF4, DCAF7, |
| | DCAF8, DDX11, DDX17, DDX18, DDX3Y, DDX58, DDX6, DEF6, |
| | DENND1B, DENND1C, DENND2D, DENND3, DENND4A, DERL1, |
| | DERL3, DGCR14, DGKA, DGKH, DGKZ, DHRS13, DHRS9, DHX34, |
| | DIAPH1, DICER1, DNAJB9, DNAJC3, DNM2, DOCK10, DOCK8, DOK2, |
| | DSC2, DVL2, DYNC1H1, DYNLT1, DYRK2, E2F5, EAF2, EBLN3, |
| | EDEM1, EFHD2, EGLN3, EHD2, EHMT2, EIF1AY, EIF3K, EIF4E3, ELF1, |
| | ELF4, ELMSAN1, EMB, EML2, EML4, ENTPD5, EOMES, EPHA2, EPRS, |
| | EPSTI1, ERGIC1, ERICH1, ERLEC1, ERN1, ERO1L, ERO1LB, ERP27, |
| | ERP29, ETS1, ETV6, ETV7, EVI2A, EVI2B, EYA3, EZR, FUR, F13A1, |
| | FAIM3, FAM101B, FAM107B, FAM117A, FAM120AOS, FAM122A, |
| | FAM129A, FAM175B, FAM185A, FAM192A, FAM214A, FAM217B, |
| | FAM222B, FAM26F, FAM46C, FAM60A, FAM65A, FAM65B, FAM78A, |
| | FAM84B, FANCD2, FANCF, FAR2, FAS, FBRSL1, FBXL16, FBXL20, |
| | FBXO44, FBXO9, FBXW7, FCHSD2, FCN1, FCRL3, FCRL5, FCRLA, |
| | FEZ2, FGD2, FGD3, FGFR1OP2, FICD, FKBP11, FKBP1A, FKBP2, |
| | FKBP8, FLT3LG, FMNL2, FNBP1, FNBP4, FNIP1, FNTA, FOXN2, |
| | FRAT1, FXR1, FYB, FZD2, FZD6, G2E3, GALM, GAS2L3, GATA3, |
| | GBGT1, GBP1, GBP1P1, GBP2, GBP4, GBP5, GCDH, GCH1, GCNT7, |
| | GDAP2, GFI1, GFRA2, GHDC, GIGYF1, GIMAP1, GIMAP2, GIMAP4, |
| | GIMAP6, GIMAP7, GIMAP8, GIT2, GJD3, GLS, GLT1D1, GLYR1, GMFG, |
| | GNA13, GNAS, GNB1, GNB5, GNG2, GNG7, GNLY, GNRH1, GOLPH3L, |
| | GOLT1B, GON4L, GPANK1, GPATCH8, GPR132, GPR155, GPR160, |
| | GPR171, GPR18, GPR183, GPR84, GPSM3, GRK6, GSK3A, GSK3B, |
| | GTF2A1, GTPBP1, GUCD1, GUSBP11, GVINP1, GYG1, GZMA, GZMB, |
| | GZMH, GZMK, H1FX, H2AFY, H2BFS, HAPLN3, HCST, HECTD1, HELZ, |
| | HERC5, HERC6, HERPUD1, HERPUD2, HIC2, HIPK1, HIPK2, HIST1H1C, |
| | HIST1H2BK, HIST1H4C, HLA-A, HLA-B, HLA-C, HLA-DOB, HLA- |
| | DPA1, HLA-DQB1, HLA-DRB6, HLA-E, HLA-F, HLA-G, HLA-J, HM13, |
| | HMGA1, HMGB1, HMGB2, HMGXB3, HMHA1, HMMR, HNF1B, |
| | HNRNPAO, HNRNPH1, HNRNPK, HNRNPUL1, HOPX, HPS1, HS3ST3B1, |
| | HSD17B11, HSH2D, HSPA13, ICAM1, ICAM3, IDH2, IER5, IFFO2, |
| | IFI27L1, IFI6, IFIH1, IFNAR2, IFNGR1, IFNLR1, IGFL2, IGFLR1, IGH, |
| | IGHD, IGHG1, IGHM, IGHV1-2, IGHV1-46, IGHV3-21, IGHV3-23, |
| | IGHV3-47, IGHV4-28, IGHV4-31, IGHV4-34, IGJ, IGK, IGKC, IGKV1D-8, |
| | IGKV4-1, IGLC1, IGLJ3, IGLL3P, IGLV1-40, IGLV1-44, IGLV2-14, |
| | IGLV3-1, IGLV3-10, IGLV3-19, IGLV3-25, IGLV4-3, IGLV4-60, IGLV5- |
| | 45, IGLV6-57, IGLV7-43, IKBKE, IKZF1, IKZF3, IL12RB1, IL15, IL15RA, |
| | IL16, IL17RA, IL18BP, ILIA, IL21R, IL27RA, IL2RB, IL2RG, IL32, IL4I1, |
| | IL6R, IL7R, INO80B, INO80D, INPP4B, INPP5D, INSIG2, INTS6, IPCEF1, |
| | IQGAP2, IRAK3, IRAK4, IREB2, IRF1, IRF2, IRF2BP2, IRF4, IRF7, IRF8, |
| | IRF9, ISG20, ITCH, ITGA4, ITGA8, ITGAL, ITGAX, ITGB2, ITGB2-AS1, |
| | ITGB7, ITK, ITM2C, ITPKB, JAK1, JAK2, JAK3, JAKMIP1, JARID2, |
| | JMJD1C, JUNB, JUND, KANSL1, KAT6B, KCNJ2, KCNN3, KCTD11, |
| | KDM2A, KDM4B, KDM5A, KDM5D, KDM7A, KIAA0125, KIAA0907, |
| | KIAA1033, KIAA1143, KIAA1147, KIAA1551, KIF2A, KLC4, KLF13, |
| | KLHDC3, KLHDC7B, KLHL24, KLHL6, KLRB1, KMO, KMT2A, KMT2C, |
| | KNOP1, KNTC1, KPNA1, KPNA3, KPNA4, KRAS, KYNU, L3MBTL2, |
| | LAIR2, LAMP3, LAMP5, LAT, LAX1, LBR, LCK, LCORL, LCP1, LCP2, |
| | LEAP2, LEF1, LEPROTL1, LGALS2, LGALS3, LIME1, LIN7B, |
| | LINC00672, LINC00847, LINC00936, LINC00957, LINC01215, |
| | LINC01278, LINC01578, LMNB1, LOC100130100, LOC100131043, |
| | LOC100190986, LOC100505650, LOC100506100, LOC100996740, |
| | LOC101927402, LOC101929272, LOC101929280, LOC101930071, |
| | LOC102723809, LOC102724699, LOC286238, LOC374443, LOC643733, |
| | LOC728613, LPXN, LRFN1, LRMP, LRP10, LRRC8C, LRRC8D, LRRFIP1, |
| | LSM12, LSM14A, LSP1, LSR, LTB, LTN1, LY6K, LY75, LY9, LYL1, |
| | LYSMD2, LYST, MALAT1, MALT1, MANEA, MAP3K1, MAP4K1, |
| | MAPK1IP1L, MAPKAPK2, MARCH6, MARCKS, MARK4, MAT2B, |
| | MATK, MAU2, MAX, MB21D1, MBD2, MBD4, MBD6, MBNL1, MBNL2, |
| | MBP, MCL1, MCM5, MCUR1, MDM4, MED1, MED12, MED13L, MED25, |
| | MEF2C, MEF2D, MEGF9, MEI1, MESDC1, MFHAS1, MFNG, MGA, |
| | MGAT2, MGEA5, MIAT, MICB, MID1IP1, MIF4GD, MIR142, MIR155HG, |
| | MIS18BP1, MLEC, MLLT10, MLX, MMP1, MMP3, MOB1A, MOB3A, |
| | MOGAT2, MPHOSPH8, MPZL3, MREG, MRPL35, MRPL49, MRPS31, |
| | MS4A1, MS4A7, MSI2, MST1, MTDH, MTFP1, MTMR1, MTMR14, |
| | MTO1, MTSS1, MXI1, MYBL1, MYL12A, MYO1F, MYO1G, MZB1, |
| | N4BP1, N4BP2L1, NAAA, NAPG, NBN, NCF4, NCK1, NCOA2, NCOA3, |
| | NDE1, NDOR1, NEDD9, NEK1, NELL2, NF1, NFATC2IP, NFE2L3, |
| | NFKB2, NFKBIA, NFKBIE, NGLY1, NIN, NIPA1, NKG7, NKTR, |
| | NLGN4Y, NLK, NLRC3, NLRC5, NLRP3, NMI, NMT1, NPAT, NRAS, |
| | NRROS, NT5C3A, NT5DC1, NUAK2, NUB1, NUDT14, NUMA1, NUP153, |
| | NUP210, NUP62, NXPE3, OAS2, OASL, OCIAD2, ODC1, OPN3, OPTN, |
| | ORAI2, ORMDL1, OSBPL3, OSTF1, OTUD5, P2RX1, P2RY10, P2RY12, |
| | P2RY13, P2RY8, PABPC1, PAG1, PAK2, PAQR8, PARP12, PARP14, |
| | PARP8, PARVG, PASK, PATL2, PAX5, PBX2, PCBD2, PCBP2, PCED1B, |
| | PCGF1, PCGF5, PCMTD1, PCMTD2, PDCL, PDE7A, PDK1, PDP1, PELI1, |
| | PHACTR1, PHACTR2, PHF1, PHF3, PHF8, PHYKPL, PIAS1, PIK3CB, |
| | PIK3CD, PIK3CG, PIK3IP1, PIM2, PIM3, PIP5K1A, PITPNC1, PKN2, |
| | PLA2G2D, PLA2G7, PLAC8, PLCG1, PLCG2, PLEK, PLEKHF2, |
| | PLEKHG3, PLEKHJ1, PLEKHO1, PML, PNISR, PNKD, PNOC, PNPLA6, |
| | PNRC1, POC5, POGK, POLB, POLD3, POLR1D, POU2AF1, POU2F2, |
| | PPAPDC1B, PPM1A, PPM1B, PPM1K, PPP1R11, PPP1R12A, PPP1R12B, |
| | PPP1R15A, PPP1R16B, PPP1R18, PPP1R35, PPP2R5C, PPP6R1, PRB1, |
| | PRDM1, PRDM2, PRF1, PRKACB, PRKCB, PRKCH, PRKCQ, PRKD2, |
| | PRKD3, PRODH, PRPF40A, PRPS2, PRPSAP1, PRR11, PRR14, PRRC2C, |
| | PSAT1, PSMB8, PSMB8-AS1, PSMB9, PSME1, PTBP3, PTGDR, PTOV1- |
| | AS2, PTPN1, PTPN2, PTPN4, PTPN6, PTPN7, PTPRC, PTPRCAP, PTPRE, |
| | PTPRO, PTTG1, PUS7L, PVRIG, PVRL2, PYHIN1, QPCT, QRSL1, QSER1, |
| | R3HDM4, RAB11FIP1, RAB11FIP4, RAB14, RAB27A, RAB30, RAB35, |
| | RAB8B, RAB9A, RABGAP1L, RAC2, RAD17, RALBP1, RALGDS, |
| | RALGPS2, RAP2A, RAP2C, RAPGEF6, RASA2, RASGEF1A, RASGRP1, |
| | RASGRP2, RASSF2, RASSF3, RASSF4, RASSF5, RBCK1, RBL2, |
| | RBM12B, RBM25, RBM27, RBM38, RBM39, RBM47, RBM5, RBM6, |
| | RCAN3, RCHY1, RCOR3, RCSD1, REC8, REL, RELB, RFTN1, RFX5, |
| | RGS1, RHBDD1, RHBDF2, RHOF, RHOH, RIC1, RILP, RILPL2, RIPK3, |
| | RIT1, RLF, RMND5A, RNASE6, RNASEH2B, RNASET2, RNF122, |
| | RNF130, RNF138, RNF144B, RNF167, RNF19B, RNF213, RNF4, RNF44, |
| | RNGTT, RNPC3, RORA, RPS4Y1, RPS6KA1, RPTOR, RSBN1L, RSF1, |
| | RTP4, RUNX3, RUSC1-AS1, SAMD3, SAMD9, SAMD9L, SAMHD1, |
| | SAMSN1, SASH3, SATB1, SBNO2, SCAF1, SCAF11, SCAMP2, SCFD1, |
| | SCML4, SDAD1, SDC1, SDCCAG3, SDF2L1, SDK1, SEC11C, SEC14L1, |
| | SEC24A, SEC62, SECISBP2, SECISBP2L, SEL1L, SEL1L3, SELK, SELL, |
| | SELPLG, SEMA4A, SEMA4D, SEPT1, SEPT6, SEPT7, SERP1, SERPINA1, |
| | SERPINB9, SET, SETD1B, SETD5, SF3A2, SF3B1, SGPL1, SH2D1A, |
| | SH2D3C, SH3BP1, SIAH1, SIGIRR, SIGLEC10, SIPA1, SIPA1L3, SIRT1, |
| | SIT1, SKAP1, SLA, SLA2, SLAMF1, SLAMF6, SLAMF7, SLAMF8, |
| | SLC12A7, SLC12A9, SLC15A2, SLC15A4, SLC1A4, SLC22A14, |
| | SLC25A28, SLC25A29, SLC25A36, SLC2A3, SLC2A6, SLC30A7, |
| | SLC35D2, SLC35F2, SLC38A1, SLC39A8, SLC40A1, SLC44A1, SLC45A4, |
| | SLC7A1, SLC7A5, SLC7A6OS, SLC8A1, SLFN5, SMARCB1, SMCHD1, |
| | SMDT1, SMYD2, SNAP23, SNIP1, SNORA61, SNRK, SNRNP25, SNX20, |
| | SNX27, SOCS1, SOD2, SON, SOS1, SP1, SP100, SP110, SP140, SP140L, |
| | SP4, SPAG4, SPATA13, SPATA2L, SPATS2, SPCS1, SPCS2, SPCS3, |
| | SPEN, SPI1, SPIB, SPN, SPOCK2, SPPL3, SQSTM1, SRGAP2, SRGN, |
| | SRPK1, SRPK2, SRPR, SRRM1, SRRM2, SRSF7, SRSF9, SS18L2, SSH1, |
| | SSR4, ST3GAL1, ST6GAL1, STAC3, STAMBPL1, STAP1, STARD5, |
| | STARD7, STAT1, STAT2, STAT4, STIM2, STK10, STK17A, STK17B, |
| | STK24, STK26, STK4, STRBP, STXBP2, SUB1, SUPT5H, SURF1, |
| | SUV39H1, SUZ12, SVIP, SYF2, SYK, SYNE1, SYNE2, SYNRG, SYTL1, |
| | SYVN1, SZRD1, TAGAP, TAOK1, TAP1, TAP2, TAPBP, TBC1D10A, |
| | TBC1D10C, TBC1D14, TBC1D2B, TBC1D8, TBC1D9, TBL1XR1, TBX21, |
| | TC2N, TCEB3, TCF25, TCF3, TCF4, TCF7, TES, TFAM, TFEB, TFG, |
| | TFRC, TGFB1, TGIF2, TGOLN2, TGS1, THAP11, THEMIS, THUMPD3- |
| | AS1, TIFA, TIGD5, TIGIT, TKT, TLE4, TLN1, TLR10, TLR8, TMC6, |
| | TMC8, TMCC3, TMED4, TMED8, TMEM173, TMEM19, TMEM194A, |
| | TMEM229B, TMEM256, TMEM33, TMEM55B, TMEM70, TMOD2, |
| | TMPO, TMSB10, TNFAIP3, TNFAIP8, TNFAIP8L2, TNFRSF10A, |
| | TNFRSF14, TNFRSF17, TNFRSF1B, TNFSF10, TNFSF13B, TNIK, TNIP1, |
| | TNKS2, TNPO1, TNRC6A, TNRC6B, TNRC6C-AS1, TOP1, TOP2A, |
| | TOR3A, TOX, TOX4, TP53INP1, TPD52, TPM4, TPR, TRA2B, TRABD2A, |
| | TRAC, TRADD, TRAF1, TRAF3, TRAF3IP3, TRAM1, TRAPPC10, |
| | TRAT1, TRBC1, TRDC, TRG-AS1, TRIB1, TRIB3, TRIM21, TRIM22, |
| | TRIM23, TRIM33, TRIM52, TRIM59, TRIM69, TRIO, TRPV5, TSHZ1, |
| | TSPAN13, TSPAN14, TSPAN33, TSPAN5, TSTD1, TTC39C, TUBA4A, |
| | TULP4, TXLNGY, TXNDC11, TXNIP, UBA5, UBA6, UBALD2, |
| | UBASH3A, UBD, UBE2B, UBE2C, UBE2D2, UBE2G1, UBE2H, UBE2J1, |
| | UBE2L6, UBE2N, UBE2W, UBE2Z, UBE3A, UBE3C, UBN1, UBN2, |
| | UBQLN1, UBXN4, UCP2, UGCG, UHMK1, UIMC1, USF2, USO1, USP1, |
| | USP15, USP3, USP33, USP9Y, UTP23, UVRAG, VAC14, VAMP1, |
| | VAMP5, VCPIP1, VNN1, VNN2, VOPP1, VPREB3, VPS13B, VPS52, |
| | VPS8, VRK1, VRK3, VTI1A, WAC, WARS, WAS, WASF2, WBP11, |
| | WDFY1, WDFY4, WDR54, WDTC1, WEE1, WHSC1L1, WIPF1, WNK1, |
| | WTAP, WWP1, XAB2, XBP1, XCL1, XPC, XRN1, YDJC, YME1L1, |
| | YPEL2, YPEL3, YWHAE, YY1, ZAP70, ZBED1, ZBP1, ZBTB2, ZBTB38, |
| | ZBTB44, ZC3H12D, ZC3H4, ZCCHC3, ZDHHC18, ZFAND6, ZFAT, |
| | ZFP36L2, ZFY, ZFYVE28, ZMYND15, ZMYND8, ZNF101, ZNF107, |
| | ZNF200, ZNF215, ZNF253, ZNF274, ZNF275, ZNF292, ZNF33B, |
| | ZNF385A, ZNF513, ZNF518B, ZNF567, ZNF581, ZNF609, ZNF641, |
| | ZNF652, ZNF655, ZNF664, ZNF688, ZNF709, ZNF720, ZNF75A, ZNF791, |
| | ZNF831, ZNF93, ZYX |
| Down- | AACS, AADAT, AAGAB, AAMDC, AASS, ABCA11P, ABCA3, ABCB6, |
| regulated in | ABCC3, ABCC6, ABCD4, ABCE1, ABCF2, ABHD2, ABHD5, ABHD6, |
| Synovium | AB 12, ABL2, ABR, ACACA, ACADVL, ACBD6, ACER3, ACKR3, |
| RA vs. OA | ACOX1, ACOX2, ACP1, ACSL3, ACSL4, ACTL6A, ACTR10, ACTR1A, |
| | ACTR3B, ACTRT3, ADAM10, ADAM23, ADAM9, ADAMTS15, |
| | ADAMTS16, ADAMTS3, ADAMTS5, ADAMTS6, ADAMTS9, |
| | ADAMTSL1, ADAMTSL2, ADCY2, ADGRA3, ADGRG2, ADGRL1, ADI1, |
| | ADIPOR2, ADK, ADM5, ADO, ADORA3, ADSSL1, AFF4, AGFG1, |
| | AGPAT4, AGPAT6, AGPS, AGT, AHCYL1, AHCYL2, AHI1, AHNAK2, |
| | AIFM1, AIG1, AIMP2, AK1, AK3, AK4, AKIP1, AKR7A2, AKR7A3, |
| | AKT3, ALAD, ALDH18A1, ALDH1A3, ALDH6A1, ALDH7A1, ALDOA, |
| | ALG13, ALG8, ALKBH2, ALKBH3, ALMS1, AMFR, ANAPC5, ANGEL2, |
| | ANGPTL1, ANGPTL2, ANGPTL4, ANK3, ANKH, ANKLE2, ANKMY2, |
| | ANKRD13C, ANKRD35, ANKRD50, ANO10, ANO5, ANPEP, ANXA2, |
| | ANXA2P1, ANXA2P2, ANXA5, AOC2, AP1B1, AP2S1, AP4M1, APC, |
| | API5, APITD1, APLP2, APOE, APOPT1, APP, APPBP2, APTX, AQP1, |
| | AQR, AR, ARCN1, ARG2, ARHGAP21, ARHGAP28, ARHGAP5, |
| | ARHGAP5-AS1, ARHGEF10, ARHGEF12, ARHGEF37, ARIH2, ARL1, |
| | ARL13B, ARL2, ARL3, ARL4D, ARL6, ARL6IP5, ARMC9, ARMCX1, |
| | ARMCX2, ARMCX3, ARMCX4, ARPC1A, ARRB1, ARSD, ASAH1, |
| | ASAP3, ASB1, ASNSD1, ASPH, ASS1, ATAD1, ATE1, ATG12, ATIC, |
| | ATL2, ATL3, ATP1A1, ATP5F1, ATP5J, ATP5S, ATP6AP1, ATP6V0E1, |
| | ATP6V1E1, ATP8B1, ATPAF1, ATR, ATRN, ATXN2, AUTS2, AZI2, |
| | B3GALNT1, B3GALTL, B3GAT3, B3GNT7, B4GALT2, B4GALT4, |
| | B4GAT1, BACE1, BAG1, BAG2, BAG4, BAG5, BAIAP2, BBS12, BBS9, |
| | BBX, BCAP29, BCAP31, BCAR1, BCAT1, BCCIP, BCKDHB, BCL2L2, |
| | BCL6, BCR, BDH2, BDKRB2, BECN1, BEGAIN, BEND6, BHLHB9, |
| | BICC1, BIRC7, BIVM, BLZF1, BMP1, BMP4, BMP8A, BMP8B, BMPR1A, |
| | BMS1, BNC2, BNIP1, BPHL, BRCC3, BRD9, BRK1, BTBD11, BTBD3, |
| | BTC, BTG3, BUD31, BYSL, BZW1, C10orf2, C11orf57, C11orf70, |
| | C11orf74, C11orf95, C12orf29, C12orf43, C14orf132, C14orf28, C14orf39, |
| | C15orf52, C16orf45, C17orf70, C17orf85, C17orf89, C1GALT1, |
| | C1GALT1C1, C1orf122, C1orf123, C1orf216, C1orf52, C1QBP, C1R, C1RL, |
| | C21orf59, C2orf40, C2orf68, C2orf69, C2orf74, C4orf27, C4orf3, C5orf15, |
| | C5orf42, C6orf120, C6orf132, C6orf203, C6orf89, C7orf25, C7orf73, |
| | C8orf33, C8orf46, C8orf88, CAAP1, CACNA1C, CACNA2D1, CALD1, |
| | CALHM2, CALU, CAMK2D, CAMKK2, CAMSAP1, CAMSAP2, |
| | CAMTA2, CAND1, CAPN7, CAPS, CAPS2, CARD14, CARKD, CASC3, |
| | CASC4, CASD1, CASP7, CASP9, CBR4, CBX1, CBY1, CCAR2, CCDC113, |
| | CCDC149, CCDC174, CCDC25, CCDC7, CCDC80, CCNB1IP1, CCND1, |
| | CCNT2, CCNY, CCSER2, CCT2, CCT5, CCT6A, CCT7, CD164, CD276, |
| | CD300C, CD320, CD55, CD59, CD63, CD81, CD9, CD99L2, CDADC1, |
| | CDC123, CDC23, CDC27, CDC42BPA, CDC42EP2, CDH23, CDK7, |
| | CDKN1C, CDKN2B, CDO1, CDON, CEBPD, CEBPZOS, CEP112, CEP126, |
| | CEP162, CEP41, CEP63, CEP70, CEP78, CERS2, CERS4, CES1, CETN2, |
| | CETN3, CFAP36, CFAP69, CFDP1, CFI, CGREF1, CHCHD1, CHCHD3, |
| | CHCHD4, CHD1L, CHD9, CHEK2, CHIC1, CHID1, CHMP1B, CHMP2B, |
| | CHRNA3, CHST3, CHSY1, CIAO1, CIAPIN1, CINP, CIR1, CISD1, CLCC1, |
| | CLIC4, CLIC6, CLMP, CLN8, CLOCK, CLTC, CLTCL1, CLU, CLUAP1, |
| | CLUL1, CLYBL, CMAS, CMSS1, CMTM4, CNIH1, CNTLN, CNTN4, |
| | COA7, COBL, COG5, COG7, COL12A1, COL14A1, COL1A2, COL22A1, |
| | COL3A1, COL6A2, COL8A2, COMMD2, COMP, COPS6, COPZ2, COQ3, |
| | CORO1C, COX20, CPNE1, CPNE3, CPNE4, CPQ, CPS1, CPSF3, CPT2, |
| | CRADD, CRAMP1L, CRAT, CREB5, CRK, CRLF1, CRLS1, CRNDE, |
| | CROT, CRTAC1, CRTAP, CRYM, CSDE1, CSE1L, CSGALNACT1, |
| | CSNK1A1, CSPG4, CSTF1, CTBP2, CTDSP1, CTDSPL, CTNNA1, |
| | CTNNAL1, CTPS1, CTPS2, CTSA, CTSF, CTTN, CUL4A, CUL4B, CUX1, |
| | CWC22, CWC27, CXADR, CXorf36, CXXC5, CYBRD1, CYCS, CYFIP1, |
| | CYP27C1, CYP2U1, CYP4V2, CYP4X1, CYP51A1, CYR61, DAAM1, |
| | DAB2, DAB2IP, DAD1, DAG1, DAP, DARS, DAW1, DBT, DCAF10, |
| | DCAF13, DCAF16, DCAF5, DCAF6, DCBLD2, DCTD, DCUN1D4, |
| | DCUN1D5, DDAH1, DDHD2, DDO, DDR2, DDX1, DDX10, DDX19A, |
| | DDX21, DDX24, DDX31, DDX3X, DDX49, DDX5, DDX50, DDX55, |
| | DECR2, DEDD, DEFB124, DENND4C, DENR, DET1, DGCR2, DGUOK, |
| | DHCR24, DHFRL1, DHX29, DHX33, DHX37, DHX38, DHX57, DHX9, |
| | DIO2, DIP2C, DIRAS3, DIS3L, DISP1, DKC1, DLAT, DLC1, DLG5, DLX3, |
| | DLX4, DMAP1, DNAJC10, DNAJC12, DNAJC15, DNAJC21, DNAJC25, |
| | DNAJC30, DNAJC8, DNAL1, DNAL4, DNALI1, DNASE1L3, DNM3OS, |
| | DOCK1, DOHH, DOK4, DOPEY1, DPCD, DPH5, DPH6, DPP8, DPT, |
| | DPY19L1, DPY19L3, DPYSL2, DPYSL3, DROSHA, DSEL, DSPP, DST, |
| | DSTN, DTD1, DTWD1, DUS4L, DUSP27, DYNC1LI2, DYNC2LI1, |
| | DYNLRB1, DYNLT3, DYX1C1, DZIP1, EAF1, EARS2, EBF1, EBF2, |
| | ECHDC2, ECM2, EDEM3, EEF1A1, EEF1E1, EFCAB2, EFEMP1, |
| | EFEMP2, EFNA5, EFNB2, EFS, EFTUD1, EI24, EID1, EIF1, EIF1AX, |
| | EIF2S2, EIF3B, EIF3F, EIF3I, EIF3J, EIF3L, EIF3M, EIF4E, EIF4E2, |
| | EIF4G1, EIF4G2, EIF5, EIF5B, EIF6, ELAC1, ELN, ELOVL4, ELP2, ELP3, |
| | ELP4, ELP5, ELP6, EMC1, EMC2, EMC3, EMC4, EMCN, EML1, EMP1, |
| | EMP2, ENAH, ENDOD1, ENO1, ENOSF1, ENOX1, ENOX2, ENPP1, |
| | ENPP4, ENPP5, ENPP6, ENY2, EPB41L5, EPCAM, EPHA3, EPHB2, |
| | EPM2A, EPS8L2, ERC1, ERCC1, ERCC6L2, EREG, ERLIN1, ERLIN2, |
| | ERMAP, ERRFI1, ESD, ESF1, ESYT2, ETAA1, ETF1, ETFA, ETFB, |
| | ETFDH, EVA1C, EXO5, EXOC2, EXOC6B, EXOSC5, EXOSC7, EXOSC9, |
| | EXT2, EXTL2, EYA2, F5, FABP3, FAH, FAHD1, FAHD2A, FAM110B, |
| | FAM110C, FAM114A1, FAM118B, FAM120B, FAM120C, FAM134B, |
| | FAM162A, FAM168A, FAM168B, FAM172A, FAM173B, FAM174A, |
| | FAM178A, FAM19A5, FAM200A, FAM200B, FAM20B, FAM20C, |
| | FAM210B, FAM219B, FAM228B, FAM229B, FAM35A, FAM43B, |
| | FAM46A, FAM46B, FAM49A, FAM50A, FAM63B, FAM73A, FAM84A, |
| | FAM92A1, FAM98A, FAM98B, FAP, FARP1, FARSB, FAT4, FBLN5, |
| | FBN1, FBXL4, FBXO22, FBXO28, FBXO32, FBXW2, FCGRT, FDX1, |
| | FERMT2, FFAR4, FGD5, FGF14, FGF18, FGF2, FGFR1, FH, FHIT, FIBP, |
| | FIGN, FILIP1, FITM2, FKBP14, FKBP1B, FKBP7, FKTN, FLJ37035, |
| | FLNB, FMOD, FN1, FNDC4, FNIP2, FOXC1, FOXJ3, FOXO1, FOXP1, |
| | FOXP2, FRA10AC1, FRK, FRS2, FTL, FTSJ2, FUBP3, FUCA2, FUNDC1, |
| | FUNDC2, FXN, FZD1, FZD10-AS1, FZD7, FZD8, GAA, GABARAPL1, |
| | GABPA, GABRA4, GABRB1, GABRB2, GADD45B, GADD45GIP1, |
| | GAL3ST4, GALNT10, GALNT11, GALNT5, GAN, GAPLINC, GAREM, |
| | GAREML, GART, GATAD1, GATAD2A, GATC, GBAP1, GBAS, GCAT, |
| | GCFC2, GCHFR, GCSH, GDF5, GDPD5, GEMIN2, GEMIN6, GEMIN8, |
| | GFM1, GFPT1, GFPT2, GFRA1, GGCT, GGCX, GGPS1, GID8, GJA1, |
| | GJA5, GJB2, GJB6, GK5, GLCE, GLG1, GLI3, GLIDR, GLIS3, GLMP, |
| | GLRB, GLT8D2, GMDS, GMPR2, GNAL, GNAQ, GNG11, GNG12, GNL1, |
| | GNL2, GNL3, GNPNAT1, GOLGA3, GOLM1, GOPC, GOSR2, GPAA1, |
| | GPALPP1, GPATCH4, GPBP1, GPC4, GPC5, GPER1, GPR1, GPR107, |
| | GPR153, GPR88, GPRASP1, GPRC5A, GPRC5C, GPSM1, GPSM2, GPX8, |
| | GRB10, GRB2, GREB1, GRHL1, GRHPR, GRIA3, GRK4, GRK5, GRSF1, |
| | GSPT1, GSTA4, GSTM3, GTDC1, GTF2A2, GTF2H5, GTPBP4, GUCA1A, |
| | GUCY1A2, GUF1, H2AFV, H6PD, HABP4, HACD3, HADH, HADHA, |
| | HAS1, HAS2, HAUS2, HAUS7, HBEGF, HCFC2, HDAC2, HDAC4, |
| | HDAC8, HDDC2, HDGFRP3, HDHD1, HDHD2, HDLBP, HEATR1, |
| | HECTD2, HEG1, HEPH, HERC2, HERC4, HES1, HEXA, HEXIM1, HFE, |
| | HGF, HGSNAT, HHIP, HIBADH, HIBCH, HIP1, HLCS, HLTF, HMGA2, |
| | HMGCL, HMGCS1, HMGN5, HNRNPD, HNRNPU, HNRNPUL2, |
| | HOMER1, HOMER3, HOOK2, HOTAIRM1, HOXA10, HOXA5, HOXB6, |
| | HOXB7, HOXD4, HOXD8, HP, HPGDS, HR, HRH1, HS6ST2, HSBP1, |
| | HSD17B14, HSDL1, HSDL2, HSP90AA1, HSPA12A, HSPA4L, HSPA9, |
| | HSPE1, HTATSF1, HTR2A, HTRA1, HTRA3, HTRA4, HYMAI, IARS2, |
| | ICE2, ICMT, IDH3B, IFI16, IFRD2, IFT122, IFT22, IFT43, IFT46, IFT57, |
| | IGF1, IGF1R, IGFBP2, IGFBP5, IGIP, IGSF10, IGSF3, IKBKAP, IL11RA, |
| | IL13RA1, IL13RA2, IL17D, IL17RC, ILF3-AS1, ILVBL, IMMP1L, IMP3, |
| | IMPAD1, IMPDH2, ING3, ING4, ING5, INHBA, INO80, INPP1, INPP5F, |
| | INTS7, IPO5, IPO9, IPP, IQCE, IRAK1BP1, ISM1, ITFG1, ITGAV, |
| | ITGB1BP1, ITGB3, ITGB5, ITGB8, ITGBL1, ITIH4, ITPA, ITSN1, IVD, |
| | IVNS1ABP, JAZF1, JMJD6, KAL1, KANK2, KANK4, KARS, KATNAL1, |
| | KAZN, KBTBD4, KBTBD6, KCND3, KCNJ5, KCNMA1, KCNQ1, KCNQ3, |
| | KCNQ5, KCTD10, KCTD2, KCTD3, KDELC2, KDELR2, KDELR3, |
| | KDM6A, KDSR, KHDRBS3, KIAA0141, KIAA0368, KIAA0895, |
| | KIAA1217, KIAA1324L, KIAA1429, KIAA1586, KIAA1644, KIAA1671, |
| | KIAA1715, KIF13A, KIF1B, KIF3B, KIF5A, KIF5B, KIF7, KIFAP3, |
| | KIRREL, KLC1, KLF10, KLF3-AS1, KLHDC1, KLHL20, KLHL22, |
| | KLHL29, KLHL42, KLHL9, KPNA6, KPNB1, KREMEN1, KRR1, KRT10, |
| | L1TD1, LAMB2, LAMP1, LAMTOR2, LAPTM4A, LAPTM4B, LARS, |
| | LCA5, LCLAT1, LDHA, LDOC1L, LGALS1, LGI2, LGMN, LGR4, LIG3, |
| | LILRA2, LILRB5, LIMA1, LIMCH1, LIN7C, LINC00116, LINC00476, |
| | LINC00657, LINC00674, LINC00938, LINC01003, LINC01088, |
| | LINC01137, LINC01268, LINC01279, LINC01503, LINC01560, LIPT1, |
| | LLPH, LMBR1, LOC100130705, LOC100132167, LOC100133039, |
| | LOC100133315, LOC100289058, LOC100289097, LOC100506730, |
| | LOC101927151, LOC101927668, LOC101927752, LOC101927811, |
| | LOC101928307, LOC102606465, LOC102724927, LOC103344931, |
| | LOC200772, LOC286272, LOC440982, LOC646762, LOC729680, |
| | LOC730102, LONRF3, LPAR1, LPAR4, LPCAT3, LPGAT1, LPP, LPPR2, |
| | LRP1, LRP11, LRP12, LRP1B, LRP6, LRPAP1, LRPPRC, LRRC2, |
| | LRRC37A3, LRRC58, LRTOMT, LSG1, LSM4, LSM5, LTBP3, LTC4S, |
| | LUC7L, LYNX1, LYRM2, MAGED2, MAGEE1, MAGI1, MAGI2-AS3, |
| | MAGI2-IT1, MAGI3, MAGOH, MAN1B1, MAN1C1, MAN2B2, MANBAL, |
| | MANSC1, MAP1A, MAP1LC3A, MAP2K4, MAP2K5, MAP3K2, MAP3K4, |
| | MAP3K7, MAP4, MAP4K5, MAP7D3, MAP9, MAPRE2, MARCH2, |
| | MARK1, MARK3, MARVELD1, MAST2, MATR3, MBLAC2, MCAT, |
| | MCFD2, MCM8, MCOLN3, MDH2, ME3, MECOM, MECR, MED21, |
| | MED29, MED7, MED8, MEDAG, MEG3, MEG9, MEIS2, MEIS3P1, |
| | MERTK, METAP1D, METAP2, METRN, METTL10, METTL22, |
| | METTL25, METTL2B, METTL5, METTL6, METTL9, MFAP3L, MFI2, |
| | MFSD7, MGC24103, MGC27345, MGP, MGST3, MIA3, MIB1, MICU3, |
| | MIEF1, MINA, MINOS1, MIPEP, MIR100HG, MIR143HG, MIR181A2HG, |
| | MIR22HG, MIR99AHG, MKKS, MKL2, MKLN1, MKS1, MLF1, MLH3, |
| | MLLT1, MLPH, MLYCD, MMAA, MN1, MNAT1, MOB1B, MOCS2, |
| | MORC4, MORN2, MPC2, MPDZ, MPHOSPH10, MRAP2, MRC2, MRO, |
| | MRPL1, MRPL17, MRPL19, MRPL20, MRPL24, MRPL3, MRPL30, |
| | MRPL32, MRPL42, MRPL46, MRPL51, MRPS16, MRPS17, MRPS30, |
| | MSANTD4, MSN, MSRB2, MSTO1, MSX1, MT1X, MT4, MTAP, MTCH2, |
| | MTERF2, MTF1, MTHFD1L, MTMR11, MTMR2, MTURN, MTUS1, |
| | MTX3, MUC1, MUT, MXRA7, MYO10, MYO1B, MYO6, MYO9A, MYOF, |
| | MZF1, N6AMT2, NAA50, NACA, NAE1, NALCN, NAP1L1, NAP1L3, |
| | NAP1L5, NAV1, NBL1, NBPF3, NCBP1, NCBP2, NCEH1, NCKAP1, |
| | NCR3LG1, NDEL1, NDFIP1, NDFIP2, NDN, NDP, NDUFA10, NDUFA5, |
| | NDUFAF1, NDUFB2, NEDD4L, NEDD8, NEFH, NEIL2, NELLI, NENF, |
| | NEO1, NET1, NFAT5, NFATC3, NFATC4, NFE2L1, NFIA, NFIB, NFYB, |
| | NGEF, NGF, NGFR, NGRN, NHLRC2, NHLRC3, NIP7, NMD3, NME1, |
| | NNMT, NOL10, NOLC1, NOP16, NOV, NOVA1, NOX4, NPHP3, NPR2, |
| | NPR3, NPRL3, NPTN-IT1, NR4A2, NRAV, NRBF2, NRP2, NSDHL, NSF, |
| | NSMCE1, NSRP1, NT5C3B, NT5E, NTMT1, NTN4, NTNG1, NTRK2, |
| | NUBP2, NUBPL, NUCB2, NUCKS1, NUDCD1, NUDCD3, NUDT11, |
| | NUDT12, NUDT15, NUDT21, NUDT3, NUDT9, NUP133, NUP155, NUP35, |
| | NUPL1, NUPR1, NXF3, OBSL1, OCIAD1, OCRL, OGFOD3, OIP5-AS1, |
| | OLFM1, OLR1, OPA1, OSCP1, OSER1-AS1, OSGEPL1, OSMR, OSR1, |
| | OSTM1, OTUD7B, OXCT1, OXR1, P3H2, P4HA1, P4HB, PACRGL, |
| | PACSIN2, PAF1, PAFAH1B1, PAFAH1B2, PAFAH2, PAICS, PAIP1, |
| | PALM2, PAM, PAPPA, PAQR3, PAQR5, PARD3B, PARK7, PARN, |
| | PART1, PARVA, PAWR, PAXIP1, PBDC1, PC, PCCA, PCDH7, PCDH9, |
| | PCDHB14, PCDHB15, PCDHB16, PCDHB2, PCDHB6, PCGF2, PCM1, |
| | PCNX, PCOLCE, PCSK1, PCSK5, PCSK6, PDCD2, PDCD5, PDCD6, |
| | PDE1A, PDE1C, PDE3A, PDE4D, PDE4DIP, PDE8A, PDGFD, PDGFRA, |
| | PDHB, PDHX, PDIA3, PDK3, PDLIM4, PDLIM7, PDPN, PDPR, PDXDC1, |
| | PDXK, PDZD8, PDZRN4, PEBP1, PEG10, PEG3, PENK, PER3, PEX1, |
| | PEX10, PEX11A, PEX13, PEX6, PEX7, PGAP1, PGAP3, PGBD1, PGR, |
| | PGRMC2, PHAX, PHB, PHBP19, PHF10, PHF20, PHKB, PHLDA2, |
| | PHLDB1, PHLDB2, PHTF1, PHTF2, PHYH, PHYHIP, PI4K2A, PIAS2, |
| | PIBF1, PID1, PIGB, PIGC, PIGF, PIGK, PIGL, PIGM, PIGP, PIGW, |
| | PIK3C3, PIK3R5, PINK1, PIP4K2B, PITPNA, PITPNM3, PITRM1, PITX1, |
| | PKIB, PKNOX1, PKP1, PLA2G12A, PLA2G2A, PLAA, PLAC9, PLAGL1, |
| | PLAU, PLAUR, PLCB1, PLCB4, PLCD4, PLCE1, PLEKHA1, PLEKHA4, |
| | PLEKHG2, PLEKHG4, PLEKHH2, PLIN3, PLIN5, PLRG1, PLXDC2, |
| | PLXNA4, PLXNC1, PM20D2, PMP22, PMPCA, PNN, PNO1, PNPLA4, |
| | PNPO, POFUT1, POLE3, POLE4, POLR1C, POLR2E, POLR2F, POLR2G, |
| | POLR2K, POLR3A, POLR3F, POLR3H, POMGNT1, POMZP3, PON2, |
| | POP4, POU3F3, PP12719, PPA2, PPAP2A, PPARA, PPIC, PPIE, PPIP5K1, |
| | PPM1L, PPP1R16A, PPP1R36, PPP1R7, PPP2CA, PPP3CA, PPP5D1, |
| | PPP6R3, PRDM4, PRDM5, PRDM6, PRDX6, PRELID2, PRELP, PREP, |
| | PREPL, PRICKLE2, PRKAA1, PRKAG1, PRKAR2A, PRKDC, PRKG1, |
| | PRKXP1, PRMT6, PROS1, PRPF31, PRR5L, PRRC2B, PRRX1, PRSS23, |
| | PRSS35, PRTFDC1, PRTG, PRUNE2, PSD3, PSEN2, PSMA2, PSMA7, |
| | PSMB5, PSMC1, PSMC2, PSMC5, PSMD1, PSMD2, PSMD9, PTCRA, |
| | PTEN, PTGFRN, PTGR1, PTGR2, PTGS1, PTK2, PTP4A2, PTPDC1, |
| | PTPN14, PTPN21, PTPN3, PTPRD, PTPRF, PTPRG, PTPRH, PTPRM, |
| | PTPRS, PTRF, PURA, PUS1, PYGB, PZP, RAB11A, RAB11FIP5, RAB1A, |
| | RAB21, RAB22A, RAB23, RAB2A, RAB31, RAB34, RAB3GAP1, |
| | RAB40C, RAB42, RAB7B, RABGEF1, RAD50, RADIL, RALGAPA1, |
| | RAN, RANBP2, RANBP6, RAPH1, RARS, RARS2, RASSF8, RASSF8- |
| | AS1, RBAK, RBBP9, RBFOX2, RBM28, RBM8A, RBMS1, RBMS2, |
| | RBMS3, RBSN, RCAN1, RCAN2, RDH11, RECK, RECQL, RELL1, RER1, |
| | REV3L, RFC2, RGMA, RGN, RGS4, RHEB, RHOBTB3, RIF1, RIOK2, |
| | RMDN2, RNASE4, RNASEH1, RNASEH2C, RND3, RNF10, RNF11, |
| | RNF145, RNF150, RNF180, RNF217, RNF24, RNF7, RNH1, ROCK2, |
| | ROM1, RPA1, RPA4, RPGRIP1L, RPL22, RPL23AP32, RPL3, RPL37, |
| | RPL37A, RPL5, RPL6, RPL7, RPP14, RPP40, RPRD1A, RPS15A, RPS23, |
| | RPS25, RPS4X, RPS6KA2, RRN3, RRP1, RRP12, RRP15, RRP36, RRP9, |
| | RRS1, RSP02, RSRC1, RSU1, RTCB, RTFDC1, RTN4IP1, RTN4RL1, |
| | RUFY1, RUFY3, RUNDC3B, RUNX1T1, RUVBL1, RWDD1, RWDD2B, |
| | RWDD4, RYK, S100A10, S100A3, S100A4, S1PR3, SACS, SALL2, |
| | SAMD5, SAMD8, SAP18, SAP30L, SAR1A, SASH1, SAV1, SAYSD1, |
| | SBSPON, SCARA3, SCARA5, SCFD2, SCG2, SCIN, SCOC, SCP2, SCRG1, |
| | SCRIB, SCRN2, SDC2, SDF2, SDHAF2, SDHAF3, SDSL, SEC11A, |
| | SEC16B, SEC23IP, SEC63, SEH1L, SELENBP1, SELO, SELP, SEMA3A, |
| | SEMA3C, SEMA3D, SEMA3E, SEMA4C, SEMA6D, SENP6, SEP15, |
| | SEPHS1, SEPT8, SERBP1, SERF2, SERINC1, SERPINA3, SERPINB6, |
| | SERPINF1, SERTAD2, SERTAD4, SESN2, SETD7, SETD8, SETMAR, |
| | SF3B6, SFN, SFRP1, SFRP2, SGCA, SGCB, SGCD, SH2D4A, SH3PXD2A, |
| | SH3PXD2B, SH3RF1, SH3RF3, SHC3, SHF, SHISA5, SHROOM1, SIAE, |
| | SIK3, SIPA1L1, SIPA1L2, SIX1, SIX2, SIX3, SIX3-AS1, SIX4, SKIL, |
| | SKP1, SLAIN2, SLC10A3, SLC11A1, SLC16A1, SLC16A2, SLC16A7, |
| | SLC17A7, SLC19A2, SLC1A1, SLC20A2, SLC22A4, SLC23A2, |
| | SLC25A11, SLC25A13, SLC25A17, SLC25A27, SLC25A3, SLC25A37, |
| | SLC25A46, SLC26A2, SLC27A5, SLC2A10, SLC2A12, SLC2A13, |
| | SLC30A5, SLC30A9, SLC35A1, SLC35A2, SLC35A3, SLC35B2, SLC35B4, |
| | SLC35G1, SLC35G2, SLC36A1, SLC37A2, SLC37A3, SLC38A6, |
| | SLC39A3, SLC39A6, SLC43A2, SLC43A3, SLC45A1, SLC47A1, |
| | SLC48A1, SLC6A2, SLC6A8, SLC7A8, SLC8B1, SLC9B2, SLIT3, SLMAP, |
| | SLMO1, SLMO2, SLU7, SMAD2, SMAD3, SMAD5, SMARCA1, |
| | SMARCA4, SMARCD3, SMC6, SMG9, SMIM10L1, SMIM11, SMIM14, |
| | SMIM7, SMO, SMOC1, SMPD1, SMURF2, SNAP25, SNAPC4, SNAPC5, |
| | SNED1, SNORD114-3, SNRNP40, SNX1, SNX12, SNX19, SNX21, SNX24, |
| | SNX7, SNX9, SOBP, SOCS6, SORBS2, SORD, SORT1, SOWAHC, SOX15, |
| | SOX5, SOX8, SP2-AS1, SPA17, SPAG16, SPAG9, SPARCL1, SPATA6, |
| | SPECC1, SPG20, SPIN3, SPIRE 1, SPOCK1, SPON1, SPPL2A, SPRED2, |
| | SPTSSA, SRI, SRP14, SRP72, SRPRB, SRR, SRSF1, SRSF10, SRSF3, |
| | SRSF6, SRSF8, SS18, SSB, SSBP2, SSFA2, SSPN, SSRP1, ST13, STI4, |
| | ST20-AS1, ST3GAL3, ST3GAL6, ST5, ST6GALNAC2, ST6GALNAC5, |
| | ST6GALNAC6, STAG1, STAM2, STARD13, STARD9, STAT3, STBD1, |
| | STEAP2, STEAP3, STEAP4, STK11IP, STK19, STK3, STK38L, STMN2, |
| | STOX2, STRN, STRN3, STS, STXBP1, STXBP5, SUCLA2, SUCLG2, |
| | SUGT1, SULF1, SULF2, SULT1C2, SUMF1, SUMO1, SUN1, SUPT16H, |
| | SURF4, SUSD4, SUSD5, SUV420H1, SV2B, SWI5, SYAP1, SYBU, |
| | SYDE2, SYNC, SYNCRIP, SYNPO, SYPL1, SYT17, SYTL4, TACC1, |
| | TACC2, TAF11, TAF12, TAF13, TAF9B, TANC2, TANGO6, TAPT1, |
| | TAS2R14, TBC1D1, TBC1D16, TBC1D2, TBC1D20, TBC1D24, TBC1D32, |
| | TBC1D8B, TBCB, TBCCD1, TBCK, TBL1X, TBPL1, TBX15, TCAF1, |
| | TCAIM, TCEAL1, TCEAL2, TCEAL3, TCEAL4, TCEAL8, TCEB1, TCF12, |
| | TCF7L2, TCHH, TCTN1, TDRD3, TDRP, TENM1, TENM3, TFCP2, |
| | TFCP2L1, TFDP2, TFPI, TGFB2, TGFBR1, TGFBR3, TGFBRAP1, |
| | THAP10, THAP2, THBD, THBS3, THBS4, THEM4, THG1L, THOC5, |
| | THOC7, THRB, TIMD4, TIMM13, TIMM21, TIMM22, TIMM44, TIMM50, |
| | TIMP2, TIMP3, TM2D1, TM2D2, TM2D3, TM7SF3, TM9SF3, TMA16, |
| | TMCC1, TMCO1, TMCO3, TMED1, TMED3, TMED5, TMEM100, |
| | TMEM106B, TMEM108, TMEM110, TMEM120A, TMEM127, TMEM136, |
| | TMEM14A, TMEM160, TMEM161A, TMEM165, TMEM167A, TMEM17, |
| | TMEM185B, TMEM186, TMEM192, TMEM196, TMEM218, TMEM231, |
| | TMEM237, TMEM242, TMEM248, TMEM251, TMEM261, TMEM263, |
| | TMEM45A, TMEM47, TMEM5, TMEM52B, TMEM67, TMEM87A, |
| | TMEM87B, TMEM9, TMEM9B, TMOD1, TMTC1, TNC, TNFRSF11B, |
| | TOLLIP, TOM1L1, TOMM20, TOP1MT, TOR1B, TOX3, TP53BP2, |
| | TP53INP2, TPD52L1, TPD52L2, TPST1, TPT1-AS1, TPTEP1, TRABD2B, |
| | TRAF3IP1, TRAF3IP2, TRAK2, TRAM1L1, TRAPPC13, TRAPPC3, |
| | TRHDE, TRHDE-AS1, TRIAP1, TRIM2, TRIM44, TRIM58, TRIQK, |
| | TRMT10C, TRMT5, TRNP1, TRPC1, TRPS1, TSEN2, TSFM, TSHZ2, |
| | TSIX, TSN, TSPAN12, TSPAN15, TSPAN3, TSPAN31, TSPYL1, TSPYL4, |
| | TSR1, TSTD2, TTC1, TTC19, TTC23, TTC26, TTC28, TTC3, TTC4, TTC8, |
| | TTF1, TTL, TUB, TUBB2A, TUBB2B, TUBB3, TUBGCP5, TUFT1, TUG1, |
| | TULP3, TUSC2, TUSC3, TWISTNB, TXN2, TXNL4A, TXNRD2, TXNRD3, |
| | TYW1, U2SURP, UACA, UAP1, UBE2D3, UBE2D4, UBE2E3, UBE2G2, |
| | UBE2I, UBE2K, UBE2V2, UBE4B, UBL4A, UBR3, UBXN2A, UBXN2B, |
| | UBXN6, UBXN8, UEVLD, UGDH, UGGT2, UGP2, UHRF1BP1L, ULK2, |
| | UMPS, UNC119B, UNC13B, UNC50, UNC5B, UNC5C, UPF1, UQCRB, |
| | URB2, URGCP, URI1, UROD, USP13, USP18, USP22, USP46, USP51, |
| | USP9X, UST, UTP14A, VAPA, VAPB, VAT1, VAT1L, VCAN, VDAC1, |
| | VDAC3, VDR, VEZF1, VGLL3, VIT, VKORC1, VKORC1L1, VPS13A, |
| | VPS13D, VPS28, VPS45, VPS54, VTA1, VTI1B, WASL, WBP1L, WBP4, |
| | WBSCR22, WDFY3-AS2, WDPCP, WDR12, WDR13, WDR35, WDR36, |
| | WDR41, WDR46, WDR60, WDR61, WDR77, WDR78, WDR92, WDYHV1, |
| | WHSC1, WIF1, WIPF2, WISP2, WLS, WNT5B, WRN, WWTR1, XG, XIST, |
| | XPNPEP3, XPOT, XRCC6, XXYLT1, YBX3, YES1, YIPF2, YIPF4, YIPF5, |
| | YIPF6, YKT6, YLPM1, YWHAH, YWHAQ, ZADH2, ZAK, ZBED5, |
| | ZBTB10, ZBTB20, ZBTB47, ZBTB7C, ZC3H13, ZC3H14, ZCCHC14, |
| | ZCCHC24, ZDBF2, ZDHHC1, ZDHHC14, ZDHHC15, ZDHHC3, ZFHX3, |
| | ZFHX4, ZFHX4-AS1, ZFP1, ZFP37, ZFP62, ZFYVE16, ZHX1, ZHX3, |
| | ZIC1, ZMYM4, ZNF106, ZNF112, ZNF131, ZNF135, ZNF141, ZNF146, |
| | ZNF197, ZNF2, ZNF204P, ZNF234, ZNF25, ZNF268, ZNF271P, ZNF280D, |
| | ZNF286A, ZNF302, ZNF319, ZNF320, ZNF326, ZNF329, ZNF337, ZNF35, |
| | ZNF354A, ZNF365, ZNF37A, ZNF385B, ZNF385D, ZNF397, ZNF415, |
| | ZNF417, ZNF462, ZNF485, ZNF502, ZNF565, ZNF568, ZNF569, ZNF573, |
| | ZNF596, ZNF599, ZNF608, ZNF622, ZNF626, ZNF629, ZNF639, ZNF662, |
| | ZNF667, ZNF770, ZNF777, ZNF782, ZNF814, ZNF83, ZNF84, ZNF846, |
| | ZNF883, ZNHIT1, ZNHIT6, ZNRF1, ZPR1, ZRANB3, ZSCAN21, |
| | ZSCAN30, ZSWIM7, ZXDA |

FIGS. 141A-141C show an overview of gene expression in SLE vs OA synovium. FIG. 141A shows that DE analysis was conducted on gene expression data from SLE and OA synovium resulting in 6,496 DE genes, 2,477 upregulated in SLE and 4,019 downregulated in SLE. FIG. 141B shows that increased and decreased transcripts were each characterized by I-Scope and T-Scope (fibroblasts, synoviocytes) for prevalence of specific cell types. FIG. 141C shows that DE transcripts were also characterized by BIG-C for functional enrichment. Heatmaps in FIGS. 141B-141C shows that the figures represent the negative logarithm of the overlap p-value when odds ratio is greater than 1 by Fisher's Exact Test. Gray cells represent non-significant enrichment (p>0.05 or OR>1). A minimum p-value of 2.2e-16 was used.

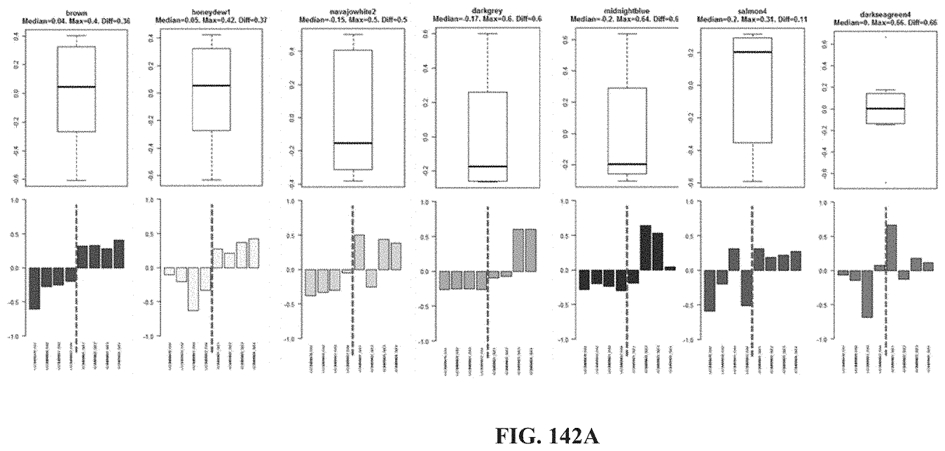

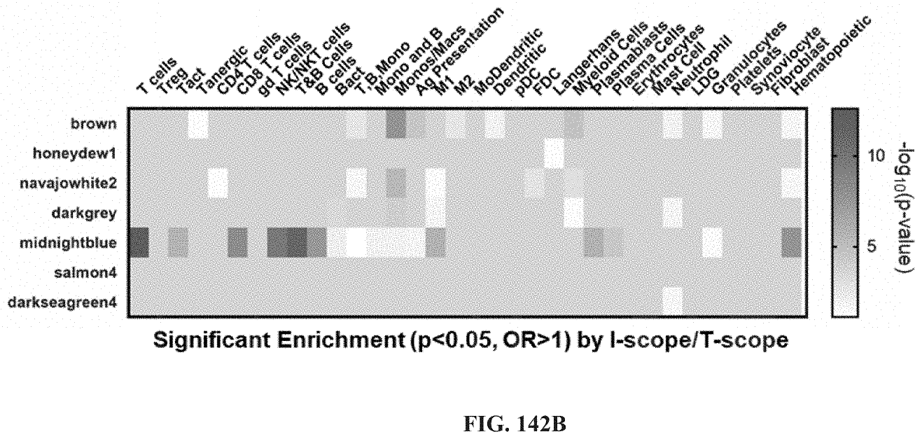

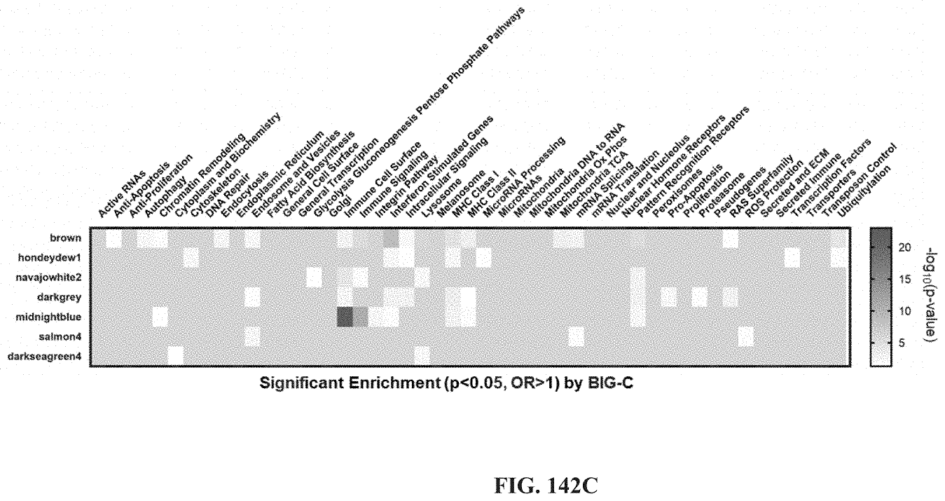

To use an orthogonal approach to identify molecular pathways dysregulated in lupus synovitis, WGCNA was carried out with the gene expression profiles from the same SLE and OA patients. The analysis yielded 52 modules of highly co-expressed genes. Of these 52, seven were chosen for further analysis based on consistent gene expression per patient in the cohort (FIG. 142A) and significant Pearson correlation coefficients to clinical metadata in the 10.5 to 11 range (Table 74). These modules included unique groupings of highly co-expressed genes were chosen for biological interrogation to investigate possible functional links to lupus synovitis.

FIGS. 142A-142C show that WGCNA reveals SLE-associated modules of genes enriched in immune cells. WGCNA of 4 SLE vs 4 OA patients yielded 7 modules of genes associated with SLE after QC and were characterized by I-Scope, T-Scope, and BIG-C. FIG. 142A shows module eigengene plots per sample of the 7 SLE-associated modules; color names are randomly generated as part of WGCNA module assignment. FIG. 142B shows that the negative logarithms of the overlap p-values identify specific immune/inflammatory cell populations or synovium-specific cell populations that may be linked to lupus synovitis or to indicate enrichment of functional gene categories (FIG. 142C). Data shown in FIGS. 142B-142C shows that the figures are significant (p<0.05) by right-sided Fisher's Exact test and must have an odds ratio above 1 to indicate enrichment.

The seven modules that are significantly correlated with features of lupus (Table 74) can be divided into two groups: modules that are positively correlated with the presence of lupus synovitis (i.e. cohort) and modules that are positively correlated with disease activity (e.g., SLE disease activity index (SLEDAI)). One module, navajowhite2, is positively correlated to lupus synovitis and SLEDAI. Interestingly, this module is also positively correlated with a marker of inflammation, CRP, and also with anti-dsDNA titer. Of note, the other two modules not significantly correlated to lupus synovitis but to SLEDAI were additionally correlated to anti-dsDNA. On the other hand, the midnightblue module is positively correlated with lupus synovitis but has significant negative correlation to both anti-dsDNA and SLEDAI. Finally, three of the SLE-correlated modules have significant negative correlations with complement components C3 and/or C4.

Table 74 shows that SLE-associated WGCNA modules correlate to clinical data. Pearson correlations of module eigengenes to clinical parameters of SLE and OA patients in the study. R values are colored red if positive or blue if negative when significant (p<0.05). Color names are randomly generated as part of WGCNA module assignment.

TABLE 74
SLE-associated WGCNA modules correlate to clinical data

| | | | | | Anti- | | | | | | | |
|---|---|---|---|---|---|---|---|---|---|---|---|---|
| | cohort | | SLEDAI | | dsDNA | | C3 | | C4 | | CRP | |
| Module | r | p | r | p | r | p | r | p | f | p | r | p |
| brown | [symbol] | [symbol] | 0.239 | 0.569 | 0.0662 | 0.876 | 0.0914 | 0.830 | 0.375 | 0.360 | 0.437 | [symbol] |
| honeydew1 | [symbol] | 0.00212 | 0.470 | 0.240 | 0.268 | 0.522 | 0.875 | 0.00445 | 0.598 | 0.117 | [symbol] | 0.0959 |
| navajowhite2 | [symbol] | 0.00315 | [symbol] | 0.00187 | [symbol] | 0.00653 | 0.928 | [symbol] | 0.314 | 0.448 | [symbol] | 0.0468 |
| darkgrey | [symbol] | 0.0399 | 0.152 | 0.719 | 0.0436 | 0.918 | [symbol] | 0.0113 | 0.811 | 0.0145 | 0.691 | 0.0576 |
| midnightblue | [symbol] | 0.0427 | 0.924 | 0.00104 | 0.909 | 0.00177 | 0.446 | 0.268 | 0.468 | 0.242 | 0.0295 | 0.945 |
| salmon4 | 0.698 | 0.0543 | [symbol] | [symbol] | [symbol] | [symbol] | 0.5.38 | 0.169 | 0.365 | 0.374 | 0.0966 | 0.820 |
| darkseagreen4 | 0.581 | 0.131 | [symbol] | 0.00414 | [symbol] | [symbol] | 0.469 | 0.241 | 0.240 | 0.567 | 0.283 | 0.497 |

[symbol]

indicates data missing or illegible when filed

Immune cell infiltration in SLE synovitis was analyzed as follows. To characterize the immune cell infiltrate in SLE synovitis in greater detail and examine genes of importance that may not be differentially expressed, I-Scope, T-Scope, and BIG-C analysis of the SLE-associated WGCNA modules was carried out (FIGS. 142B-142C). This analysis indicated that four modules contained immune/inflammatory (FIG. 142B) signatures. The four modules with the greatest immune/inflammatory cell enrichment, brown, navajowhite2, darkgrey, and midnightblue, are all correlated to lupus cohort and each has a unique pattern of immune/inflammatory cell enrichment. A strong enrichment of predominantly myeloid cell populations was evident in the brown module with monocytes/macrophages, specifically M2, as well as antigen presenting cells and numerous markers of myeloid cells and subtypes. On the other hand, midnightblue, which was also enriched for monocytes/macrophages and antigen presentation, was only significantly enriched for M1 macrophages rather than M2. Interestingly, midnightblue was the only SLE-correlated module to be significantly enriched for multiple lymphoid populations. Activated T cells, effector T cells and NK cells, B cells, and plasmablasts/plasma cells were all significantly enriched in this module. Further investigation into the plasma cell population revealed heavy chain genes IgG1, IgM, and IgD, indicating both pre-switch and post-switch plasmablasts/plasma cells as well as the presence of Igκ, Igλ, and numerous VL chains, indicating a polyclonal population (Supplemental Figure S4). Navajowhite2 and darkgrey were both strongly enriched for monocytes and macrophages, specifically M1-polarized, myeloid cells, and neutrophils (FIG. 2B). Darkgrey was additionally enriched for activated B cells.

The remaining three modules had minimal significant enrichment of immune or inflammatory cell markers (FIG. 142B). Signaling by a neutrophil population was detected in darkseagreen4 whereas Langerhans cells were detected in honeydewl. Of note, none of the SLE-associated WGCNA modules was found to be enriched for fibroblasts or synovioctyes. Rather, a module negatively correlated with lupus synovitis was enriched in synovial fibroblasts.

After identifying cell types that may play a functional role in the pathogenesis of SLE synovitis, BIG-C enrichment analysis was performed to inform about the functional perturbation of these modules (FIG. 142C). The four aforementioned modules with significant immune cell infiltration were also enriched for immunological functions, notably immune cell surface markers and pathogenic pattern recognition. Three out of four of these modules were significantly enriched for interferon activity as well as MHC Class I antigen presentation whereas two of these modules, brown and midnightblue, were significantly enriched for immune signaling processes and MHC Class II antigen presentation. Interestingly, autophagy was found to be enriched in the brown module. A number of general cell processes were also enriched across various SLE-associated modules including transcripts related to lysosome activity, ubiquitylation & sumoylation, and endosomal processes. Moreover, two of the modules with little immune cell infiltration salmon4 and darkseagreen4, were enriched for endosome & vesicle processes, mRNA splicing, ROS protection, and lysosome activity. Honeydewl, however, was enriched for both normal cell processes including changes in cytoskeleton and ubiquitylation, and immunological functions with strong enrichment of interferon stimulated genes and MHC Class I genes.

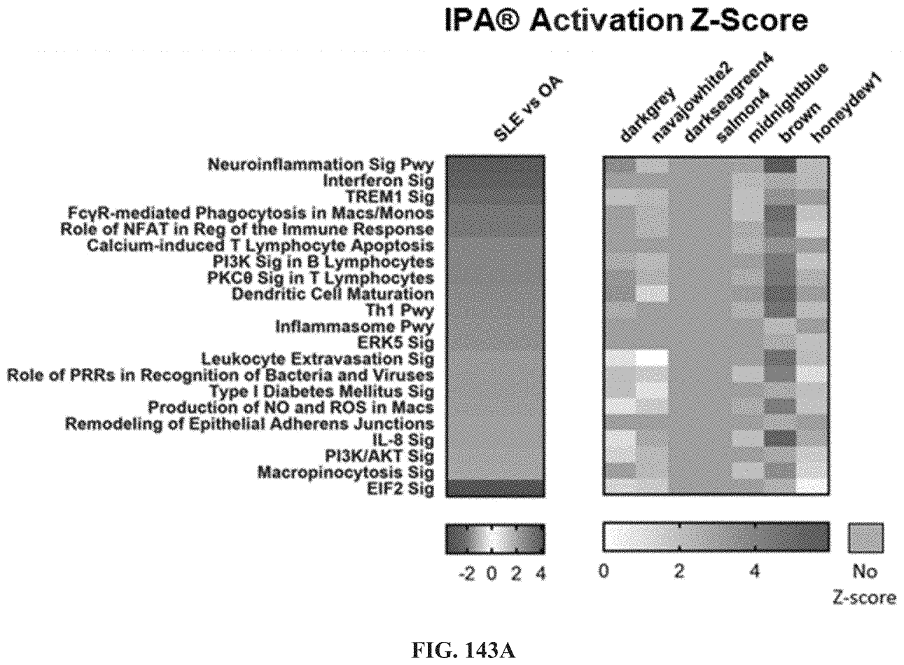

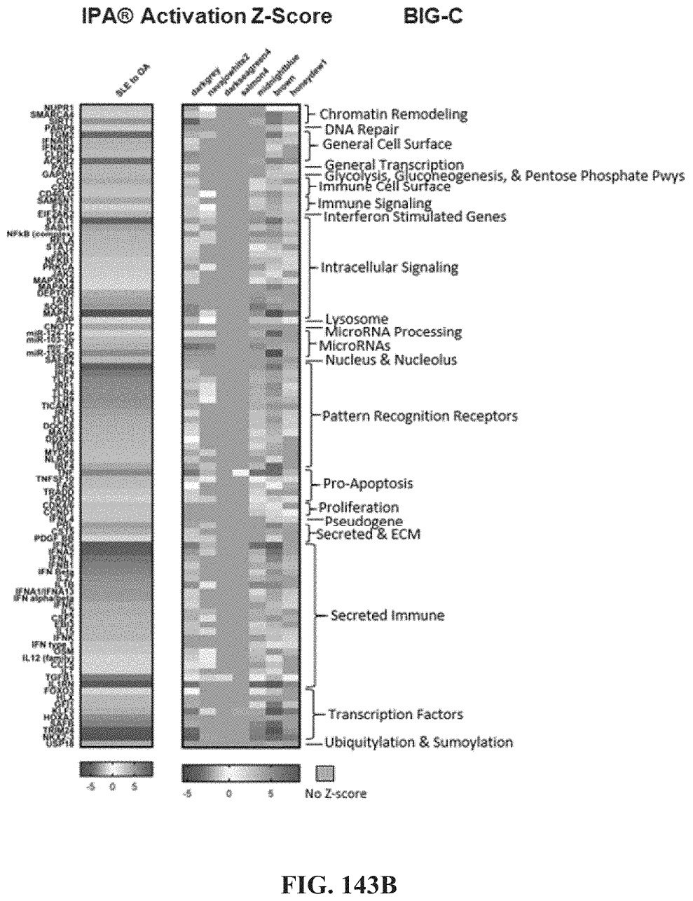

Pathogenic signaling in SLE synovitis was analyzed as follows. Next, the signaling pathways that might be activated in SLE synovitis were elucidated. IPA canonical pathway and upstream regulator analysis functions were used to assess both DE data and the WGCNA modules (FIGS. 143A-143B). Canonical pathways predicted to be significantly activated or downregulated in SLE by both DE data and at least one SLE-associated WGCNA module are outlined in FIG. 143A. Most of the consensus canonical pathways were related to processes ensuring a productive immune response (e.g. Role of NFAT in the Regulation of the Immune Response, PI3K/AKT Signaling) and innate immune mechanisms (e.g. Dendritic Cell Maturation, Role of PRRs in Recognition of Bacteria and Viruses). Canonical pathways were not identified in the darkseagreen4 and salmon4 modules, previously identified as devoid of immune/inflammatory cells (FIG. 143A).

Upstream regulators predicted to be significantly operative in SLE are outlined in FIG. 143B and include the consensus upstream regulators predicted by DE data and WGCNA modules of interest. Of the consensus upstream regulators, most fall into the BIG-C categories Intracellular Signaling, Pattern Recognition Receptors, and Secreted Immune. Of note, most secreted immune proteins are type I and type II interferons and the PRRs with the strongest activation Z-Scores are interferon regulatory factors and TLR 7/9. Interestingly, most of the transcription factor family proteins and microRNAs are predicted as negatively associated with SLE synovitis. Finally, of note, is the predicted upregulated signaling by pro-apoptotic genes TNF, TNFSF10, and FAS.

FIGS. 143A-143B show signaling pathways and upstream regulators operative in lupus synovitis. IPA canonical pathway and upstream regulator analysis was performed. FIG. 143A shows consensus canonical pathways predicted to be significantly activated or inhibited by DE transcripts and at least one SLE-associated WGCNA module. FIG. 143B shows that consensus upstream regulators predicted to be significantly activated or inhibited by both DE transcripts and at least one SLE-associated WGCNA module are displayed and organized by BIG-C category. Canonical pathways and upstream regulators were considered significant if |Activation Z-Score|≥2 and overlap p-value≤0.01.

Lymphocyte trafficking in lupus synovium was analyzed as follows. Potential pathways of immune/inflammatory cell localization to lupus synovium were identified, by analyzing DE and WGCNA data for chemokine receptor-ligand pairs and adhesion molecules. As shown in Table 75, overexpression of numerous chemokines and chemokine receptors was observed. Chemokine receptor-ligand pairs included CCR5-CCL4/5/8, CCR1-CCL5/7/8/23, and CXCR6-CXCL16, among others (Table 75). Interestingly, CCR3 and its ligands CCL7 and CCL8 were found to be co-expressed in the darkgrey module, whereas ligand CCL5 was expressed in the midnightblue module. CCL5 and CCL8 were also found to be upregulated in SLE by DE analysis. Additionally, CXCR3 and its ligands CXCL9, CXCL10, and CXCL11 were co-expressed in the midnightblue module and were all upregulated compared to OA. CXCL8, a regulator of neutrophil trafficking, was also upregulated compared to OA. Of note, CXCL13 was also expressed in the midnightblue module and upregulated by DE analysis, although its receptor CXCR5 was not detected in SLE-associated WGCNA modules nor by DE.

Table 75 shows a summary of chemokine receptor-ligand pairs and adhesion molecules. DE genes and SLE-associated WGCNA modules were assessed for adhesion molecules and chemokine receptor-ligand pairs. Receptor-ligand pairs are grouped together in the table with groupings alternately shaded. Log fold changes rounded to 3 significant figures are presented where available; otherwise, n/s=not significant.

TABLE 75
Summary of chemokine receptor-ligand pairs and adhesion molecules

| | | SLE vs OA Analysis | |
|---|---|---|---|
| Gene | | | WGCNA SLE- |
| Transcript | Name | DE LFC | associated module |
| CCL19 | Chemokine (C-C motif) ligand 19 | n/s | Honeydew1 |
| CCR2 | Chemokine (C-C motif) receptor 2 | 1.50 | Midnightblue |
| CCL2 | Chemokine (C-C motif) ligand 2 | n/s | Darkgrey |
| CCL7 | Chemokine (C-C motif) ligand 7 | n/s | Darkgrey |
| CCL8 | Chemokine (C-C motif) ligand 8 | 2.76 | Darkgrey |
| CCR5 | Chemokine (C-C motif) receptor 5 | 1.90 | Navajowhite2 |
| CCL4 | Chemokine (C-C motif) ligand 4 | 2.67 | Brown |
| CCL5 | Chemokine (C-C motif) ligand 4 | 1.94 | Midnightblue |
| CCL8 | Chemokine (C-C motif) ligand 8 | 2.76 | Darkgrey |
| CCR1 | Chemokine (C-C motif) receptor 1 | 2.14 | Brown |
| CCL5 | Chemokine (C-C motif) ligand 5 | 1.94 | Midnightblue |
| CCL7 | Chemokine (C-C motif) ligand 7 | n/s | Darkgrey |
| CCL8 | Chemokine (C-C motif) ligand 8 | 2.76 | Darkgrey |
| CCL23 | Chemokine (C-C motif) ligand 23 | 0.670 | |
| CCR3 | Chemokine (C-C motif) receptor 3 | n/s | Darkgrey |
| CCL5 | Chemokine (C-C motif) ligand 5 | 1.94 | Midnightblue |
| CCL7 | Chemokine (C-C motif) ligand 7 | n/s | Darkgrey |
| CCL8 | Chemokine (C-C motif) ligand 8 | 2.76 | Darkgrey |
| CCRL2 | Chemokine (C-C motif) | 1.04 | Navaierwhite2 |
| | receptor-like 2 | | |
| CKLF | Chemokine-like factor | 0.297 | |
| CMKLR1 | Chemokine-like receptor 1 | 1.59 | Darkgrey, Honeydew1 |
| CXCL2 | Chemokine (C-X-C motif) ligand 2 | 2.98 | Honeydew1 |
| CXCL3 | Chemokine (C-X-C motif) ligand 3 | 1.77 | |
| CXCL8 | Chemokine (C-X-C motif) ligand 8 | 2.17 | Brown, Darkgrey |
| CXCR3 | Chemokine (C-X-C motif) receptor 3 | 1.45 | Midnightblue |
| CXCL9 | Chemokine (C-X-C motif) ligand 9 | 5.59 | Midnightblue |
| CXCL10 | Chemokine (C-X-C motif) ligand 10 | 4.81 | Midnightblue |
| CXCL11 | Chemokine (C-X-C motif) ligand 11 | 3.32 | Midnightblue |
| CXCR4 | Chemokine (C-X-C motif) receptor 4 | 1.39 | Brown |
| CXCL13 | Chemokine (C-X-C motif) ligand 13 | 3.47 | Midnightblue |
| CXCR6 | Chemokine (C-X-C motif) receptor 6 | n/s | Midnightblue |
| CXCL16 | Chemokine (C-X-C motif) ligand 16 | 0.768 | Navajowhite2 |
| CXCL11 | Chemokine (C-X-C motif) ligand 11 | 3.32 | Midnightblue |
| CX3CL1 | Chemokine (C-X-C motif) ligand 1 | 0.453 | |
| XCL1 | Chemokine (X-C motif) ligand 1 | n/s | Midnightblue |
| ALCAM | Activated Leukocyte cell adhesion | 1.55 | |
| | molecule | | |
| VCAM1 | Vascular cell adhesion molecule | n/s | Navajowhite2 |
| CD44_ | CD44 molecule1 | 1.25 | Brown, Darkgrey |
| ITGB1 | Integrin subunit beta 1 | −0.255 | |
| ITGB2 | Integrin subunit beta 2 | 1.56 | Brown, Honeydew1 |
| ICAM1 | Intercellular adhesion molecule 1 | 0.861 | Darkgrey, Honeydew1, |
| | | | Midnightblue |
| ICAM3 | Intercellular adhesion molecule 3 | n/s | Midnightblue |
| PECAM1 | Platelet/endothelial cell adhesion | 0.618 | Salmon4 |
| | molecule 1 | | |
| SDK1 | Sidekick cell adhesion molecule 1 | −0.892 | |
| SDK2 | Sidekick cell adhesion molecule 3 | −1.33 | |
| CADM1 | Cell adhesion molecule 1 | −0.974 | |
| CADM3 | Cell adhesion molecule 3 | n/s | Darkgrey |
| JAM2 | Junctional adhesion molecule 2 | −0.587 | |
| JAM3 | Junction adhesion molecule 3 | −0.673 | |
| MCAM | Melanoma cell adhesion molecule | −1.08 | |

Adhesion molecules were also found to be expressed in SLE-associated WGCNA modules including VCAM1, CD44, CADM3, and ITGB2 (Table 75). These adhesion molecules and others that were co-expressed in SLE-correlated WGCNA modules tended also to be upregulated by DE analysis, whereas several other adhesion molecules were downregulated and were not expressed in SLE-associated modules. In addition, the modules in which immune cell content was relatively scarce (e.g., darkseagreen4, salmon4, and honeydewl) did not contain any co-expressed chemokine receptor-ligand pairs. Rather, CCL19 was expressed in honeydewl but its receptor CCR7 was not detected in any module, along with CXCL2 whose receptor was not found to be expressed. Darkseagreen4 did not contain any chemokine receptors, ligands, or adhesion molecules, whereas salmon4 contained only PECAM1.

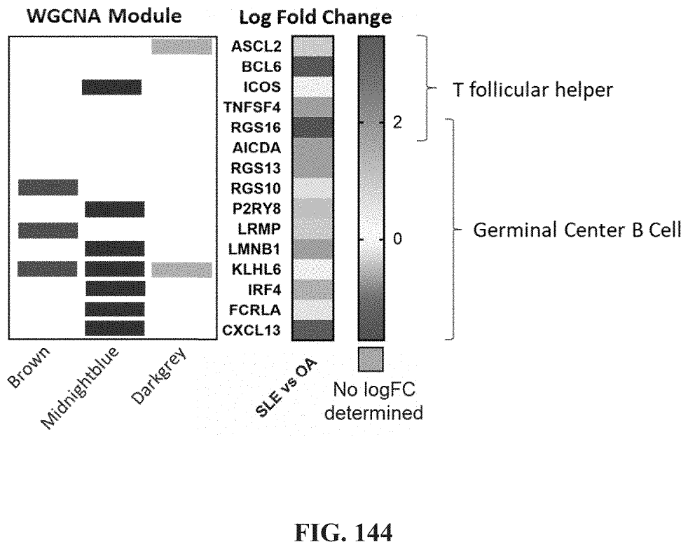

Germinal center activity in lupus synovitis was analyzed as follows. DE data and SLE-associated WGCNA modules were also examined for the expression of specific follicular helper T cells (Tfh) and germinal center (GC) B cell markers in lupus synovium to determine whether high efficiency T cell:B cell interaction may contribute to pathogenesis (FIG. 144). Several GC B cell markers were upregulated in SLE vs OA synovium, including, of importance, CXCL13 and IRF4. However, BCL6 and RGS16 were notably downregulated and RGS13 was not differentially expressed between SLE and OA. A cluster of GC B cell markers that tended to be upregulated were co-expressed in the midnightblue module.

FIG. 144 shows germinal center B cell and Tfh cell markers in lupus synovitis, including an assessment of germinal center and follicular T helper cell markers in lupus synovium from DE genes or WGCNA. Genes found in SLE-associated WGCNA modules are indicated.

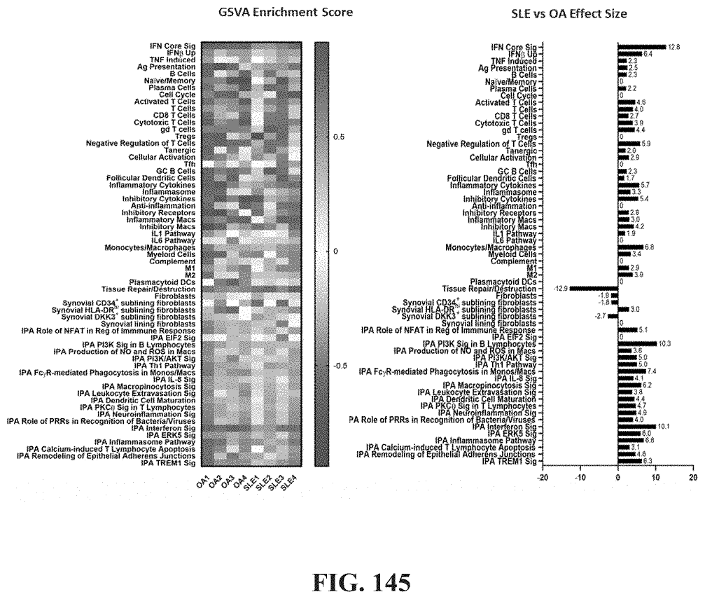

GSVA enrichment of immune populations and signaling pathways was analyzed as follows. To assess the differences between SLE and OA synovitis in greater detail and substantiate preliminary findings, GSVA of various immune cell populations and predicted IPA pathways was carried out (FIG. 145). A variety of immune cell and signaling pathway gene sets were assessed for enrichment in synovial expression data.

FIG. 145 shows that GSVA enrichment of immune populations in synovia confirms inflammatory infiltrate in SLE. GSVA of relevant immune cell populations, molecular signatures, and signaling pathways was conducted on log 2-normalized gene expression values from OA and SLE synovia. Significant differences in enrichment between cohorts were found by Welch's t-test (*p<0.05). Hedge's g effect sizes were calculated (right) with correction for small sample size for each gene set; zeroes represent non-significant differences in enrichment between cohorts. “#” indicates a literature-derived signature. Other gene set signatures were derived from IPA, where noted, PathCards, or are hand-curated lists from lupus gene expression data and literature mining.

The majority of immune and inflammatory signatures were significantly enriched in SLE compared to OA (FIG. 145). The core interferon signature, IPA PI3K signaling in B lymphocytes pathway, IPA interferon signaling pathway, IPA inflammasome pathway, and monocyte/macrophage module were the most enriched gene sets in SLE. The interferon signatures and pathways, inflammatory cytokines, monocytes/macrophages, M2 macrophages, IPA PI3K signaling in B lymphocytes, and IPA FCγR-mediated phagocytosis in monocytes/macrophages modules were most notably enriched at similar levels in all four lupus patients. Interestingly, whereas the M2 macrophage signature was similarly enriched across all lupus patients, two lupus patients had high enrichment of M1 macrophages and one had a very low M1 GSVA enrichment score. Although naïve/memory lymphocytes, cell cycle, Tregs, Tfh cells, anti-inflammation, the IL-6 pathway, and pDCs did not reach statistical significance, these gene sets also tended to be more enriched in the lupus patients. Of note, the downstream signature induced by TNF signaling was significantly enriched in lupus synovitis. On the other hand, the fibrosis signature (i.e. tissue repair/tissue destruction) was significantly diminished in SLE, along with a general fibroblast signature and two out of four specific fibroblast subsets identified in RA synovium. Synovial lining fibroblasts were found enriched in both a subset of lupus arthritis patients and in a subset of osteoarthritis patients. Synovial HLA-DRhi sublining fibroblasts were enriched in all lupus arthritis patients.

Compounds predicted to target lupus synovitis pathways were analyzed as follows. A list of drugs and compounds to offset the transcriptomic changes caused by lupus synovitis was compiled in Table 76. Table 76 summarizes the number of LINCS-predicted compounds per target category determined by connectivity scoring where at least two compounds were predicted for a given target. Most abundantly predicted compounds include anti-cancer drugs targeting tubulin polymerization, MAPK signaling, and EGFR signaling, as well as current lupus standard-of-care therapies corticosteroids and NSAIDs/prostaglandin synthesis inhibitors. Interestingly, a few alternative medicines were predicted to counteract lupus synovitis including curcumin, capsaicin, resveratrol, and caffeine.

Table 76 shows that compounds predicted by LINCS to oppose the lupus synovitis gene signature were summarized by their drug targets for every target with at least 2 compounds. Compounds were analyzed if corresponding connectivity scores fell in the range of −75 to −100 to reflect most opposite gene signatures. Top LINCS Drug represents the most negative-scoring compound for a specific target category, while Representative Drug conveys the most immunologically relevant or well-known drug for a specific target category.

TABLE 76
Compounds targeting lupus synovitis

| Target | Count | Range | Mean ± SEM | Ton LINCS Drug | Representative Drug |
|---|---|---|---|---|---|
| PKC | 5 | (−94.47)-(−99.70) | −98.01 ± 1.00 | Enzastaurin^{‡} | Midostaurin^{†0} |
| STK33 | 2 | (−94.85)-(−95.45) | −95.15 ± 0.30 | MW-STK33-1A | ML-281^{P} |
| RAF | 3 | (−89.13)-(−98.35) | −94.91 ± 2.91 | Vemurafenib^{†-6} | Sorafenib^{†-3} |
| GSK3 | 7 | (−81.19)-(−99.96) | −93.93 ± 2.57 | SB-216763^{P} | Enzastauri^{‡} |
| ROCK1/2 | 4 | (−90.80)-(−97.82) | −92.81 ± 1.68 | RHO-kinase-inhibitor-III | KD025^{†7} |
| NOS2 | 3 | (−83.31)-(−98.09) | −92.46 ± 4.61 | AR-C133057XX | Curcumin^{†7} |
| CDK | 7 | (−81.19)-(−99.96) | −91.95 ± 2.43 | SB-216763^{P} | Palbociclib^{†4} |
| GR agonist | 11 | (−83.48)-(−97.95) | −91.61 ± 1.53 | Dexamethasone^{P} | Prednisone^{†} |
| PLK | 2 | (−84.06)-(−99.04) | −91.55 ± 7.49 | GW-843682X^{P} | Rigosertib^{‡} |
| Dopamine uptake | 2 | (−86.38)-(−95.84) | −91.11 ± 4.73 | GBR-13069^{P} | Bupropion^{†} |
| (SLC6A3) | | | | | |
| Cholinesterase | 2 | (−88.16)-(−93.36) | −90.76 ± 2.60 | Mestinon^{†} | Isoflurophate^{†} |
| Retinoid receptor agonist | 5 | (−81.80)-(−95.44) | −90.34 ± 2.40 | TTNPB^{‡} | Acitretin^{†} |
| Tyrosine kinase (broad) | 4 | (−86.44)-(−97.25) | −90.24 ± 2.42 | Sorafenib^{†-3} | Nilotinib^{†0} |
| mTORC1/2 | 4 | (−88.13)-(−91.96) | −90.10 ± 0.87 | KU-0063794^{P} | N-acetyl cysteine^{†4} |
| Tubulin | 19 | (−82.65)-(−96.57) | −89.67 ± 1.10 | Epothilone^{‡} | Albendazole^{†} |
| Aurora kinase | 3 | (−81.99)-(−96.22) | −89.59 ± 4.14 | AT-9283^{‡} | Alisertib^{‡} |
| SYK | 2 | (−85.66)-(−93.43) | −89.55 ± 3.88 | Fostamatinib^{†7} | Fostamatinib^{†7} |
| EGFR | 12 | (−79.42)-(−99.14) | −89.48 ± 1.85 | Lapatinib^{†0} | Gefitinib^{†1} |
| b2 adrenergic receptor | 5 | (−82.19)-(−96.66) | −88.82 ± 2.58 | Orciprenaline | Albuterol^{†} |
| agonist | | | | | |
| 5 alpha reductase | 2 | (−86.29)-(−91.18) | −88.73 ± 2.44 | Alpha-estradiol | Acexamic acid^{†} |
| c-Met | 2 | (−81.96)-(−94.78) | −88.37 ± 6.41 | SU-11274^{P} | Cabozantinib^{†-6} |
| PARP | 6 | (−77.24)-(−98.35) | −88.28 ± 3.17 | Rucaparib^{‡} | Niraparib^{†3} |
| Angiotensin receptor | 2 | (−84.35)-(−92.20) | −88.28 ± 3.93 | Candesarta^{†} | Azilsartan^{†} |
| MDM | 5 | (−75.96)-(−95.49) | −88.13 ± 3.48 | SJ-172550^{P} | Idasanutlin^{‡} |
| MAO | 2 | (−83.30)-(−92.34) | −87.82 ± 4.52 | Nialamide\hat{ } | Bifemelane^{†} |
| Na/Ca exchange | 2 | (−86.48)-(−88.99) | −87.73 ± 1.26 | CGP-37157^{P} | CGP-37157^{P} |
| (SLC8A1) | | | | | |
| VEGFR | 4 | (−77.30)-(−97.26) | −87.70 ± 4.52 | Sorafenib^{†-3} | Sunitinib^{†1} |
| Na channel | 11 | (−79.66)-(−98.23) | −87.62 ± 1.64 | Phenamil | Benzocaine^{†} |
| TRPV agonist | 3 | (−80.20)-(−97.26) | −87.56 ± 5.06 | Capsaicin^{†} | Capsaicin^{†} |
| P450 | 3 | (−81.70)-(−92.02) | −87.53 ± 3.06 | Proadifen^{P} | Resveratrol^{†5} |
| MAP2K1/2 | 10 | (−80.30)-(−98.40) | −87.44 ± 1.93 | PD-0325901^{‡} | Vemurafenib^{†-6} |
| Androgen receptor | 5 | (−81.04)-(−96.04)0 | −87.36 ± 2.70 | BMS-641988 | Apalutamide^{†} |
| Adenosine receptorr | 2 | (−79.87)-(−94.23) | −87.05 ± 7.18 | ZM-241385^{P} | Caffeine^{†} |
| HIV protease | 2 | (−86.19)-(−87.78) | −86.98 ± 0.79 | Lopinavir | Nelfinavir^{†5} |
| HMG-CoA reductase | 5 | (−76.32)-(−93.09) | −86.97 ± 2.85 | Atorvastatin^{†3} | Statins^{†3} |
| TGFBR | 3 | (−80.73)-(−98.13) | −86.93 ± 5.61 | SB-525334 | Pirfenidone^{†} |
| Tachykinin | 3 | (−80.84)-(−91.26) | −86.39 ± 3.03 | Aprepitant^{†} | Aprepitant^{†} |
| PRKDC | 3 | (−83.42)-(−90.00) | −86.04 ± 2.02 | NU-7026^{P} | Caffeine^{†} |
| NSAID/prostagladin | 13 | (−76.19)-(−95.40) | −86.04 ± 1.58 | Phenacetin\hat{ } | Aspirin^{†} |
| MAPK | 10 | (−77.41)-(−95..39) | −85.42 ± 1.89 | FR-180204^{P} | Losmapimod^{‡} |
| TP53 | 2 | (−78.14)-(−92.39) | −85.27 ± 7.13 | Pifthrin-alpha^{P} | VLX-600^{‡} |
| Prostanoid receptor | 2 | (−82.17)-(−87.80) | −84.99 ± 2.82 | 16,16- | Alprostadil^{†} |
| agonist | | | | dimethylprostaglandin-e2^{‡} | |
| PDE | 9 | (−77.14)-(−95.12) | −84.73 ± 2.42 | Bucladesine^{†} | Dipyridamole^{†4} |
| ATM kinase | 2 | (−83.89)-(−85.10) | −84.50 ± 0.61 | CP466722 | VE-822^{‡} |
| P13K (pan) | 3 | (−78.72)-(−88.90) | −84.11 ± 2.95 | PIK-90 | Idelalisib^{†1} |
| ACE | 2 | (−81.90)-(−86.18) | −84.04 ± 2.14 | Enalapril^{†} | Alacepril^{†} |
| DHFR | 2 | (−75.51)-(−92.42) | −83.96 ± 8.45 | Pyrimethamine^{†} | Methotrexate^{†} |
| NFkB | 3 | (−76.88)-(−89.33) | −83.94 ± 3.69 | NFKB-activation- | N-acetyl cysteine^{†4} |
| | | | | inhibitor-II^{P} | |
| ALOX5 | 2 | (−76.07)-(−89.98) | −83.03 ± 6.95 | Zilueton^{†} | Diethylcarbamazine^{†} |
| DNMT | 3 | (−75.19)-(−88.82) | −82.48 ± 3.96 | Triclosan^{†} | Azacitidine^{†} |
| HDAC | 4 | (−77.36)-(−90.05) | −82.32 ± 2.75 | Valproic acid^{†2} | Vorinostat^{†6} |
| AMPA receptor agonist | 2 | (−79.16)-(−84.63) | −81.90 ± 2.74 | Nobiletin^{P} | Aniracetam^{†} |
| Topoisomerase II | 3 | (−77.89)-(−88.85) | −81.87 ± 3.50 | Razoxane | Doxorubicin^{†} |
| IGF1R | 2 | (−78.32)-(−83.82) | −81.07 ± 2.75 | GSK-1904529A^{P} | Ceritinib^{†-4} |
| HSP90AA1 | 2 | (−76.84)-(−85.28) | −81.06 ± 4.22 | Gedunin^{P} | Rifabutin^{†} |
| NAMPT | 2 | (−76.91)-(−84.58) | −80.75 ± 3.83 | FK-866^{‡} | FK-866^{‡} |
| Calcineurin | 2 | (−79.03)-(−80.44) | −79.73 ± 0.70 | Cyclosporin-a^{†} | Tacrolimus^{†5} |
| Carbonic anhydrase | 2 | (−78.54)-(−78.81) | −78.67 ± 0.13 | Chlortalidone^{†} | Acetazolamide^{†} |

^{P}Preclinical
^{‡}Drug in development/clinical trials
^{†}FDA-approved
\hat{ }Withdrawn from market

Where applicable, CoLTS scores are displayed as integers in superscript

Additional LINCS-predicted compounds were grouped by target categories for which both inhibitors/antagonists and activators/agonists were predicted to counteract the gene expression changes found in lupus synovitis.

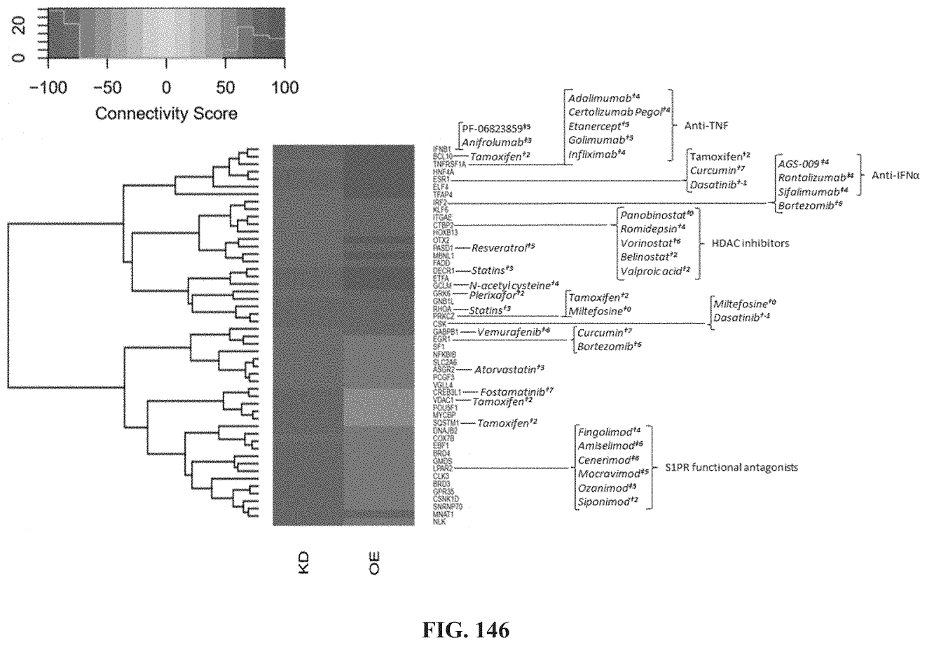

In addition to the LINCS-predicted compounds, activated signaling pathways in lupus synovitis further from the LINCS-predicted BURs were investigated. The top 50 BURs determined by connectivity scoring were summarized in FIG. 146 along with drugs that may potentially target these BURs. Finally, the upstream regulators predicted by IPA were also matched with potential drugs. Despite the lack of overlap between IPA- and LINCS-predicted upstream regulators, drug-target matching to each of the molecules in these groups supplied a vast array of compounds to be considered for drug repositioning into lupus arthritis (FIG. 146). 26% of drugs targeting IPA upstream regulators (either directly or indirectly) were also predicted by LINCS BURs drug-target matches. Most of these shared drugs were anti-TNF therapies, anti-type I interferon, NFkB pathway inhibitors, or CDK inhibitors.

FIG. 146 shows LINCS biological upstream regulators, including the top 50 targets from LINCS knockdown and overexpression data matching (overexpressed) and opposing (knocked down) the lupus synovitis gene signature. Knockdown and overexpression data were analyzed for connectivity scores in the −75 to −100 and 50 to 100 ranges, respectively. Drugs and compounds directly or indirectly antagonizing/inhibiting the biological upstream regulators were sourced from LINCS/CLUE, IPA®, literature mining, CoLTS, STITCH, and clinical trials databases. Where applicable, drug annotations are grouped together by target and CoLTS scores are displayed as integers in superscript. Indirect drug matches are displayed in italics. Only drugs with CoLTS scores are shown. “P”: Preclinical; “‡”: Drug in development/clinical trials; “†”: FDA-approved.

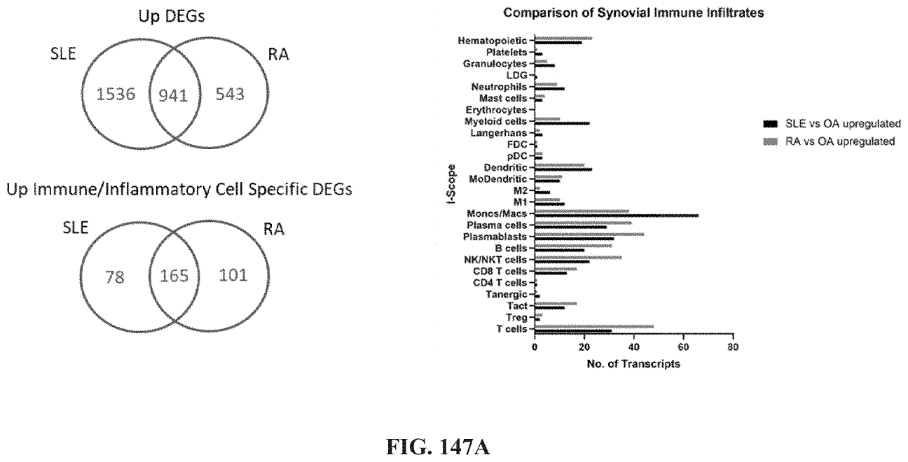

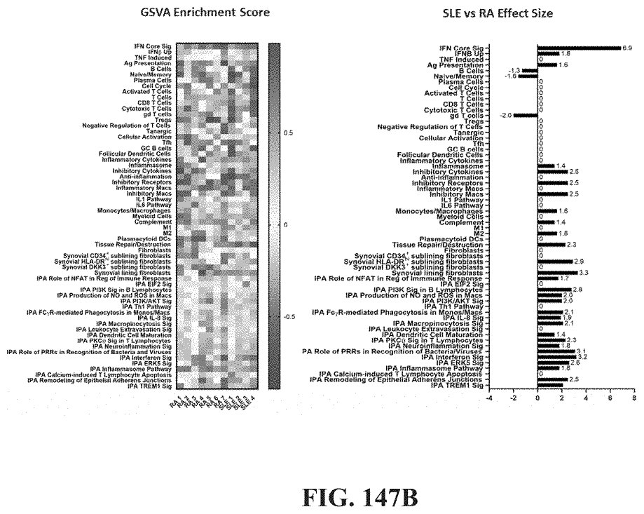

A comparison of gene expression in SLE synovitis and RA synovitis was performed as follows. To investigate the immunologic mechanisms that may differentiate lupus synovitis and RA synovitis, DEGs were also identified between 7 RA patients and 4 OA patients and then compared to SLE vs OA DEGs (FIG. 147A). A comparison of the upregulated transcripts in each disease cohort indicated that fewer genes were globally upregulated in RA. However, 18% of these genes identified immune/inflammatory cells whereas 10% of genes upregulated in SLE synovium identified immune/inflammatory cells. Upregulated DEGs were further characterized by I-Scope which revealed greater numbers of myeloid- and monocyte/macrophage-specific transcripts in SLE compared to RA Immune infiltrates in RA were more characteristic of T and B cells. GSVA of log 2-normalized gene expression levels in RA and SLE patients confirmed this relationship (FIG. 7B). GSVA also revealed enrichment of fibrosis (tissue repair/destruction) in RA but not SLE. Interestingly, two synovial fibroblast populations previously identified in RA from gene expression studies were found significantly more enriched in SLE patients compared to RA patients including synovial HLA-DRhi sublining fibroblasts and synovial lining fibroblasts.

FIGS. 147A-147B show a comparison of gene expression between SLE and RA synovitis. A comparison of immune/inflammatory and synovial gene signatures was made between SLE and RA synovium using 7 RA patients from GSE36700. FIG. 147A shows that upregulated DEGs were identified between RA and OA synovium, compared to SLE, and characterized by I-Scope. FIG. 147B shows that GSVA of immune/inflammatory cell populations, molecular signatures, and signaling pathways was carried out on log 2-normalized gene expression values from RA and SLE synovia. Significant differences in enrichment between cohorts were found by Welch's t-test (*p<0.05). Hedge's g effect sizes were calculated (right) with correction for small sample size for each gene set; zeroes represent non-significant differences in enrichment between cohorts. “#” indicates a literature-derived signature. Other gene set signatures were derived from IPA, where noted, PathCards, or are hand-curated lists from lupus gene expression data and literature mining.

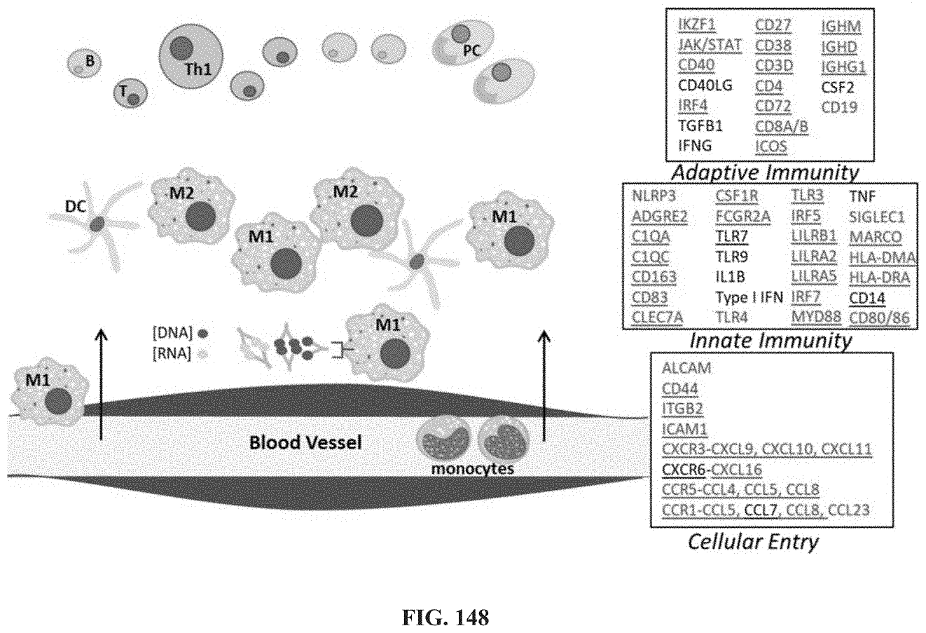

FIG. 148 shows a model of lupus synovitis. DEGs, molecules co-expressed in SLE correlated WGCNA modules, and IPA® upstream regulator predictions were integrated into a summary model of lupus synovitis. Transcripts listed on the right were either upregulated (red text), co-expressed in SLE correlated WGCNA modules (underlined), or identified as upstream regulators operative in lupus synovitis.

A multi-pronged bioinformatic and systems biology approach was performed to characterize the molecular and cellular mechanisms of inflammation in lupus synovitis. The prevalence of the interferon signature in the gene expression profile of SLE arthritis from patient-derived SLE and OA synovia may be observed, and the presence of discrete cellular infiltrates may be further characterized using immunohistochemistry. The results provided herein confirmed and further interrogated the cellular interactions and signaling pathways as they relate to lupus disease activity and other clinical parameters, and these traits were distinguished among individual patients. Ultimately, this analysis may lead to identification of novel pathways and targets that may be evaluated and investigated further for therapeutic intervention.

The initial analyses of differences in gene expression profiles between SLE and OA patients with active synovitis revealed an inflammatory infiltrate in lupus synovium of mostly myeloid lineage cell types including monocytes, M1 macrophages, antigen presenting cells, and other myeloid and hematopoietic cells Amplifying the analysis by employing WGCNA revealed enrichment of many other immune cell types, including activated and effector T cells, natural killer cells, B cells, plasma cells/plasmablasts, and both M1 and M2-polarized macrophages, indicating both innate and adaptive mechanisms at play in SLE synovitis. Subsequent analysis of the relevant inflammatory cell types by transcriptomic markers and molecular signatures by GSVA validated the enrichment of these populations in SLE compared to OA. Thus, a robust immune response is evident, including involvement of myeloid and lymphoid cell populations.

The results indicated that myeloid-lineage cells were consistently found to be enriched in SLE synovitis and, therefore, may play a central role in lupus synovitis. IPA revealed phagocytosis and NO and ROS production signaling pathways by monocytes/macrophages and GSVA confirmed gene expression profiles of both inflammatory M1 and inhibitory M2 macrophages in SLE. This raises a question as to whether these macrophages are synovial resident populations or infiltrating inflammatory cells. Histologic evaluation indicates that these are infiltrating cells, but this population was not distinguished by gene expression. The analyses indicate not only the presence of both M1 and M2 macrophage populations in lupus synovitis, but also both anti-inflammatory (e.g. IL10, IL1RN) and proinflammatory (e.g. TNF, IL1, TNFSF13B, IL18) cytokine production. Additionally, analysis of chemokine receptor-ligand pairs and adhesion molecules indicates numerous pathways for entry and retention of inflammatory cells into the lupus synovium. The macrophage subsets identified may reflect monocyte-derived proinflammatory macrophage accumulation in the joints of arthritic mice and M2-like interstitial macrophages that lose their normal protective barrier function but maintain anti-inflammatory roles. These corresponding profiles may be similar to those of pathogenic cell populations identified in human RA, and thus a similar mechanism may be occurring in lupus synovitis. Given that three out of four SLE-associated WGCNA modules containing immune cells were enriched in M1 macrophage transcripts and only one out of four enriched in M2 transcripts, there may be a bias towards inflammatory macrophages in SLE synovitis; this may be explored using approaches in addition to transcriptomics analysis. This may be consistent with analysis of myeloid cells in SLE indicating a proinflammatory M1 phenotype associated with active versus inactive disease.

In addition to macrophages, fibroblasts are important components of the synovium. None of the significantly upregulated genes or SLE-associated WGCNA modules was found to be significantly enriched for either fibroblasts or synoviocytes. Fibroblast unique genes were found among the downregulated DEGs, however. Additionally, one of the WGCNA modules that was significantly negatively correlated to lupus synovitis (and thus significantly correlated with OA) was found to be significantly enriched for fibroblasts. These groupings of genes may, therefore, represent local loss or diminished/altered function of resident fibroblasts. Pathologic fibroblast populations may reside in the synovium of patients with rheumatoid arthritis and may be identified by single cell RNA sequencing, including a subpopulation that was associated with higher expression of MHC Class II genes, IL6, and CXCL12 and may perpetuate inflammation. SLE may therefore differentiate from RA and OA wherein the latter cases joint organ pathology is characterized by fibroblast-mediated tissue degradation, a loss of function of, or dysregulation of fibroblasts from inflammation.

Of particular interest in lupus pathophysiology is the contribution of interferons. The results noted significant upregulation of interferon-inducible (IFI) genes through DE analysis confirmed by immunostaining and real-time RT-PCR. Significant enrichment of the core interferon signature (shared by all type I interferons) and ongoing signaling by type I and type II interferons were observed. Several SLE-correlated WGCNA modules were found to be strongly enriched for the interferon signature. Plasmacytoid dendritic cells (pDCs) may be reported to produce type I interferon in SLE, express high amounts of IRF7, and depend on TLR3/4 and TLR7/9 signaling to induce IRF7 expression and IFNα production, respectively. Although significant enrichment of pDCs in lupus synovium was not detected, evidence of dendritic cell (DC) activation and maturation was found, which may indicate the differentiation of pDCs into classical DCs following IFNα production.

The detection of Ig heavy chain pre- and post-switch plasma cells in lupus synovium was notable. IRF4, XBP1, and PRDM1 were all detected in the midnightblue module with LIMMA log fold changes of 1.47, −0.84, and 0.842, respectively. These transcription factors are essential to B cell maturation and development into plasma cells and can be used to identify plasmablasts/plasma cells. There was some evidence of GC formation, but BCL6, a major transcription factor involved in Tfh and GC B cell activity, was not upregulated nor contained in an SLE-associated WGCNA module. AICDA and RGS13 were also not expressed at a significant level nor detected in any SLE-associated WGCNA modules. However, CXCL13, a chemoattractant that may be reported in RA synovial GCs, was strongly upregulated in the brown module. Consequently, without the expression of key markers of GC activity BCL6, AICDA, and RGS13, rather than fully-formed GCs, it is likely that lupus synovium contains lymphoid aggregates that support B cell proliferation and autoantibody formation, as reported in the spleen in immune thrombocytopenia. Although proliferative lymphoid nodules are associated with the production of autoantibodies in immune thrombocytopenia, an interesting result from the data is the strong negative correlations of the midnightblue module, which contains the plasmablast/plasma cell signature, to SLEDAI and anti-dsDNA whilst positively correlated to lupus synovitis. This suggests that the presence of plasmablast/plasma cells in lupus synovitis does not contribute significantly to systemic autoantibody levels and extra-articular lupus disease activity. Rather, the nature of the local inflammation may facilitate entry of circulating plasmablasts/plasma cells into the synovial space and/or their local differentiation.

The overexpression of numerous chemokines and chemokine receptors indicates chemokine signaling may play a significant role in the infiltration of immune/inflammatory cells in lupus synovitis. Chemokine receptors, their ligands, and adhesion molecules were found amongst the upregulated DEGs and found to be co-expressed primarily in SLE-associated WGCNA modules identified as having significant enrichment of immune cell populations. CXCR3 and its ligands CXCL9, CXCL10, and CXCL11 were all found upregulated and co-expressed in the midnightblue module, which contained a robust lymphocyte signature. This signaling axis may be induced by IFNγ and may be involved in the recruitment of activated lymphocytes, particularly of naïve T cells and their differentiation into Th1 cells, and the migration of immune cells to their focal sites. CXCR3 and CXCR4 may be additionally important to the homing and maintenance of plasma cells. CXCR4 may also be indicative of a GC response as well as CXCL13, which were both upregulated although their respective ligands were neither up- nor downregulated. Thus, these chemokine receptors could be involved in the recruitment of circulating plasmablasts/plasma cells into lupus synovium and/or their in situ differentiation, as previously mentioned. Other chemokines and their receptors such as CCR5-CCL4/CCL5 indicate recruitment of other leukocytes into the synovium including macrophages, monocytes, and T cells.

A number of approaches were employed to utilize the gene expression analysis to predict novel drugs that might target abnormally expressed genes or pathways and suppress inflammation. Predicted drugs and compounds identified novel potential therapies, but also confirmed current treatments by identifying current standard-of-care lupus drugs, such as glucocorticoids, methotrexate, aspirin, and cyclosporine. Notably, a large number of anti-cancer drugs with variable mechanisms of action were also predicted. However, anti-cancer drugs are also standard-of-care in lupus treatment.

Drugs targeting the cyclin-dependent kinase (CDK) family were comparably high-scoring and abundant and may point to potential repurposing of drugs such as palbociclib or related seliciclib and other CDK inhibitors, for which amelioration of lupus nephritis in mouse models may be reported, as well as reduced proliferation of lupus T- and B-cells in vitro. Similarly, bucladesine was one of nine phosphodiesterase inhibitors predicted to offset lupus synovitis. Other immunopathogenic targets and signaling pathways of interest with candidate drugs for repositioning based on the LINCS predictions include KD025 targeting ROCK2, fostamatinib targeting SYK and other kinases, niraparib targeting PARP1 and PARP2, and HDAC inhibitor Vorinostat. Notably, a large number of sodium channel blockers were predicted, possibly related to the increased nervous innervation of the inflamed synovium. Neurologic targets included the acetylcholine, dopamine, serotonin, GABA-A, adrenergic, and glutamate receptors. These may have been predicted based on changes in the innervation of the inflamed tissue, although an effect on immune/inflammatory cells is also possible. Similarly, transmembrane ion channels were predicted and may reflect dysregulation of innervation or a role on immune/inflammatory cells.

Even less clear are the drug predictions surrounding the estrogen and progesterone receptors. Women may be affected by systemic lupus 10 times more often than men, and sex hormones may be involved in modulating the immune system. While glucocorticoids have mainly anti-inflammatory and immunosuppressive effects, sex hormones such as estrogen and progesterone may have either pro-inflammatory or anti-inflammatory effects depending on the types of receptors expressed and other factors. Estrogen may increase risk of disease by favoring autoreactive B cells and promoting type I interferon production, whereas progesterone seems to counteract these effects. Thus, the right balance of these hormones may attenuate disease activity. In an all-female cohort, ESR1, encoding the alpha estrogen receptor, was found to be an upstream regulator of lupus synovitis. Tamoxifen was also repeatedly suggested as a potential therapy for lupus synovitis and may show utility in murine lupus and in human lupus T cells. However, no clinical trials of Tamoxifen have been conducted in lupus or other autoimmune disease and cases of Tamoxifen-induced lupus and other adverse outcomes may be reported. Thus, female hormone receptors may be important in lupus pathogenesis, and further study may be performed to delineate their specific roles and crosstalk between glucocorticoids and sex hormones.

Comparison of gene expression in SLE synovitis and RA synovitis revealed differences in the nature of the immune infiltrate when compared to OA synovium. A greater number of genes were found significantly altered in SLE than in RA, but a smaller portion of these transcripts could be attributed to immune/inflammatory cell populations, indicating an overall greater immune infiltrate in RA than in SLE. Of the immune/inflammatory cell-specific transcripts identified, RA upregulated DEGs indicated a higher likelihood of T cells, B cells, NK/NKT cells, and other lymphocytes, while SLE upregulated DEGs were more characteristic of monocytes/macrophages and myeloid cells. Thus, SLE synovitis may be more myeloid-mediated than RA. GSVA replicated this finding with significant upregulation of the core type I interferon signature, antigen presentation signature, inflammasome pathways, and monocyte/macrophage cell populations including, notably, more inhibitors of inflammation. Interestingly, although no statistical differences were found between cohorts, the downstream TNF, IL-1, and IL-6 signatures tended to be more enriched in SLE patients than RA patients, indicating the potential for repurposing of anti-TNF biologics, anti-IL-1 anakinra, and anti-IL-6 tocilizumab to treat lupus arthritis.

Finally, the differences in enrichment among specific fibroblasts populations that may be reported in RA were notable. A population of HLA-DRhi sublining fibroblasts were found significantly enriched in all four lupus patients but not in any RA patients; however, this signature was derived from single-cell RA fibroblast gene expression data and the constitutive marker genes are mainly interferon and MHC Class II genes. Thus, while this subpopulation may have been identified as pathologic in RA synovium, the same cannot be said from bulk gene expression data in SLE. On the other hand, a population of fibroblasts in the synovial lining was found uniformly enriched in SLE compared to RA. This may indicate a diminished or loss of fibroblast function in RA but not SLE synovitis concomitant with tissue repair/damage.

Bioinformatic analysis of lupus arthritis was performed to reveal a pattern of immunopathogenesis in which myeloid cell-mediated inflammation dominates. The breadth of the immune response underlying SLE synovitis provides a basis for multiple avenues of therapeutic intervention to be considered that mouse models and previous studies have failed to provide up to this point. With these findings, specific candidate target genes and pathways from which to develop or repurpose drugs to treat and improve the condition of lupus arthritis patients may be identified and further investigated or evaluated.

### Example 39: Analysis of Discoid Lupus Erythematosus (DLE) Gene Expression Reveals Dysregulation of Pathogenic Pathways Associated with Infiltrating Immune/Inflammatory Cells

Discoid lupus erythematosus (DLE) is a chronic, scarring inflammatory autoimmune disease of the skin. The precise molecular pathways underlying DLE pathogenesis have not been fully delineated. To obtain a more complete view of the pathologic processes involved in DLE, a comprehensive analysis of gene expression profiles from DLE affected skin was performed.

Microarray gene expression data was obtained from skin biopsy samples of three studies (GSE81071, GSE72535, and GSE52471). Differentially expressed genes (DEGs) between DLE and control were identified by LIMMA analysis. Weighted gene co-expression network analysis (WGCNA) yielded modules of co-expressed genes. Modules correlating to clinical data were prioritized. Correlated modules were interrogated for statistical enrichment of immune and non-immune cell type specific gene signatures. Genes were functionally characterized using a curated immune-specific gene functional category database (BIG-C) and pathways elucidated using IPA®. Queries of a perturbation database (LINCS, Library of Integrated Network-Based Cellular Signatures) were used to identify drugs that could reverse the altered gene expression patterns in DLE.

For each dataset, between 7-12 WGCNA modules had significant correlations to disease. Significant WGCNA module preservation was observed between all three datasets. Non-immune cell types (fibroblasts, keratinocytes, melanocytes) and also Langerhans cells were represented in WGCNA modules negatively correlated with disease. An immune cell signature was observed in WGCNA modules positively correlated to DLE, including DCs, myeloid cells, CD4+ & CD8+ T cells, NK cells, B cells as well as pre- and post-switch plasma cells (PCs). The presence of both Ig-κ and -λ, as well as multiple VL genes suggests the presence of polyclonal PCs. Chemokines that mediate lymphocyte organization and/or recruitment into the skin were identified, including CCL5,7,8 and CXCL9-10,13. Cytokines (TNF, IFNγ, IFNα, IL1β, IL2, IL6, IL12, IL17, IL23, and IL27), signaling molecules (CD40L, PI3K, and mTOR) and transcription factors (NF-κB, NF-AT), as well as cellular proliferation, were evident. IPA® UPR analysis indicated that many of the expressed genes may be secondary to signaling by TNF, IFNγ, IFNα, CD40L, IL1β, IL2, IL6, IL12, IL17, IL23, and IL27. Interestingly, connectivity analysis using LINCS/CLUE identified high-priority drug targets, such as IKZF1/3 (lenalidomide, CC-220), JAK1/2 (ruxolitinib), and HDAC6 (Ricolinostat) may be viable options for therapeutic intervention.

Bioinformatic analysis of DLE gene expression has elucidated many dysregulated signaling pathways potentially involved in the pathogenesis of DLE that may be targeted by novel therapeutic strategies. Further investigation of these signatures may provide an enhanced understanding of the pathogenesis of DLE.

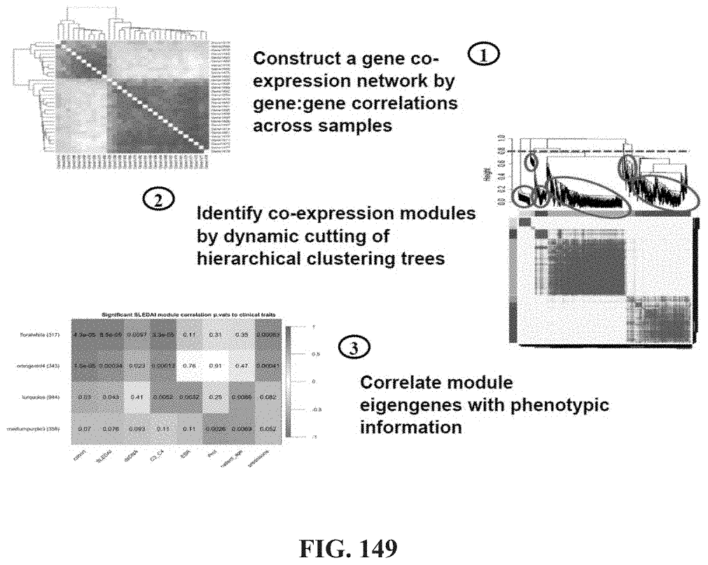

FIG. 149 shows an example of weighted gene co-expression network analysis (WGCNA) to create modules of correlated genes through hierarchical clustering, including constructing a gene co-expression network by gene:gene correlations across samples, identifying co-expression modules by dynamic cutting of hierarchical clustering trees, and correlating module eigengenes with phenotypic information.

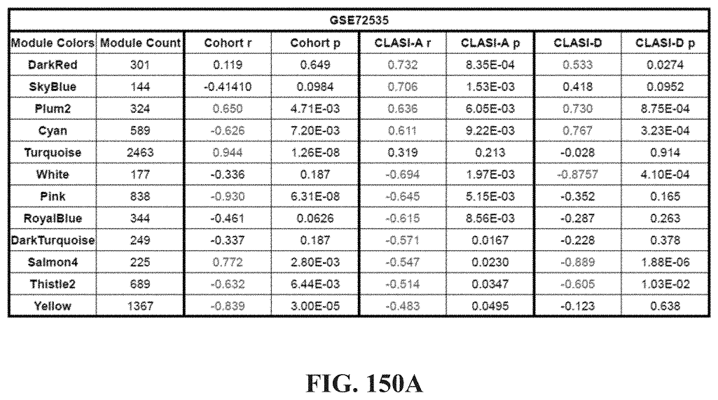

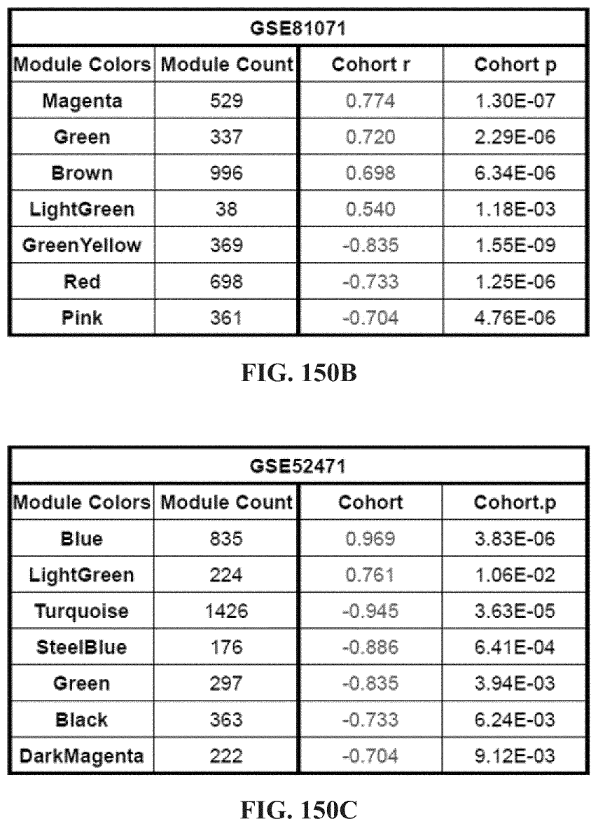

FIGS. 150A-150C show that WGCNA identified modules with significant correlations to clinical variables in DLE datasets. WGCNA identified 41 modules for GSE72535, 23 modules for GSE81071, and 30 modules for GSE52471. FIG. 150A shows that in GSE72535, 12 modules were significantly correlated to CLASI.A or cohort (5 positively and 7 negatively). FIGS. 150B-150C show that in GSE81071 (FIG. 150B) and (FIG. 150C) GSE52471, 7 modules were significantly correlated to cohort (GSE81071: 4 positively and 3 negatively; GSE52471: 2 positively and 5 negatively).

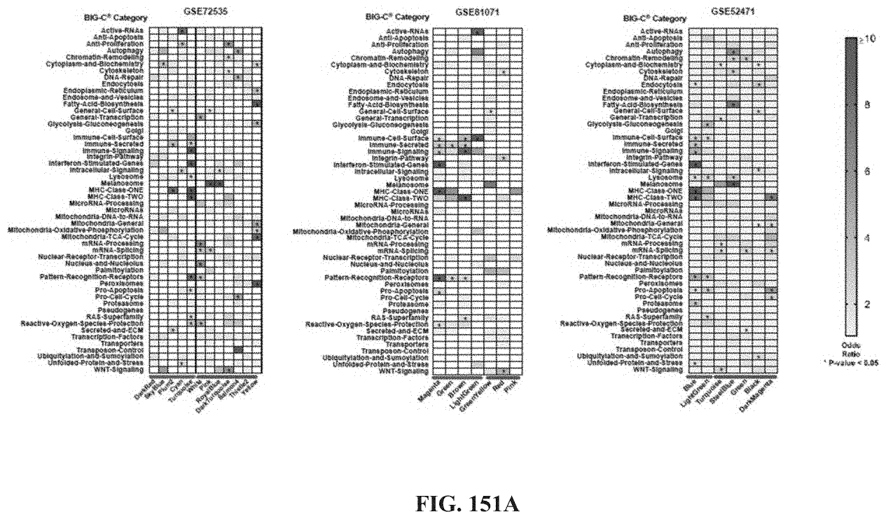

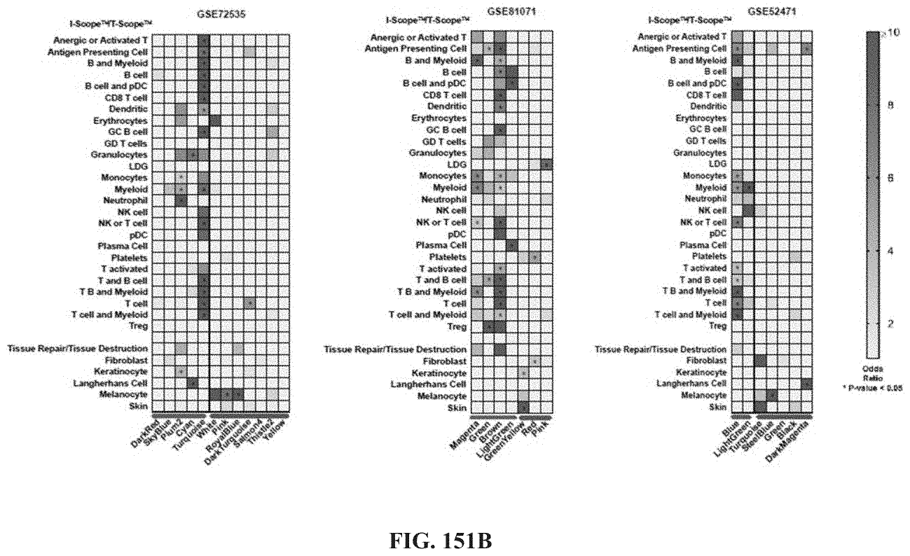

FIGS. 151A-151B show WGCNA modules interrogated using BIG-C® functional characterizations as well as I-Scope™ and T-Scope™ for specific cellular subsets. DLE-associated modules identified in WGCNA are characterized by BIG-C® (FIG. 151A) and I-Scope™/T-Scope™ (FIG. 151B). Odds ratios above 1 are shown, and Fisher's exact tests with p-values below 0.05 are indicated with an asterisk. Consistent enrichment of several categories, including immune signaling, pattern recognition receptors, and pro-apoptosis, was seen across all three analyses. Additionally, a clear immune signature, including antigen presenting cells, T cells, and myeloid cells, was observed in positively correlated modules.

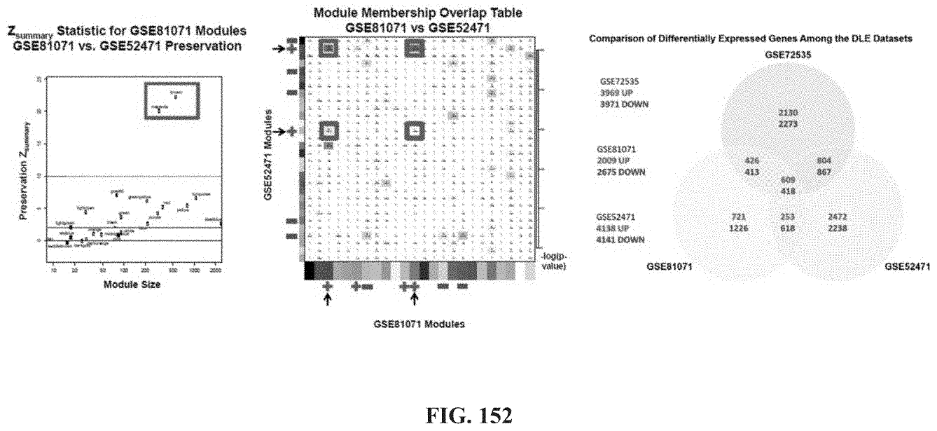

FIG. 152 shows WGCNA modules statistically preserved and common DE genes between three analyses. Module preservation was performed for each pairwise combination of datasets. The preservation Zsummary statistic was used to determine significant preservation. A representative example of the WGCNA modules from GSE81071 in the preservation analysis between GSE81071 and GSE52471. The overlap p-value (Fisher's exact test) was used to determine specific module associations between datasets. Interestingly, the analyses consistently showed the preservation of the two positively correlated modules in each dataset (Turquoise and Plum2 in GSE72535, Brown and Magenta in GSE81071, and Blue and LightGreen in GSE52471).

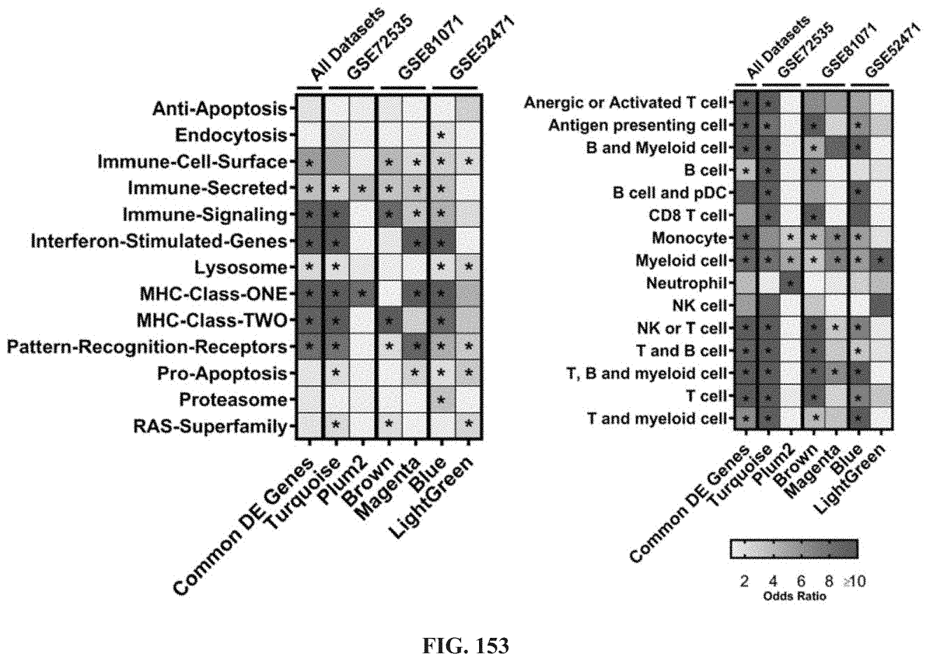

FIG. 153 shows BIG-C®, I-Scope™ and T-Scope™ analysis results in the preserved modules and common DE genes. The analysis compared DE genes common to all three datasets and the 6 preserved DLE-associated WGCNA modules. BIG-C® (left) and I-Scope or T-scope categories (right) found to have an odds ratios above 1 in both DE transcripts and at least one module from each dataset are shown. Fisher's exact tests with p-values below 0.05 are indicated with an asterisk.

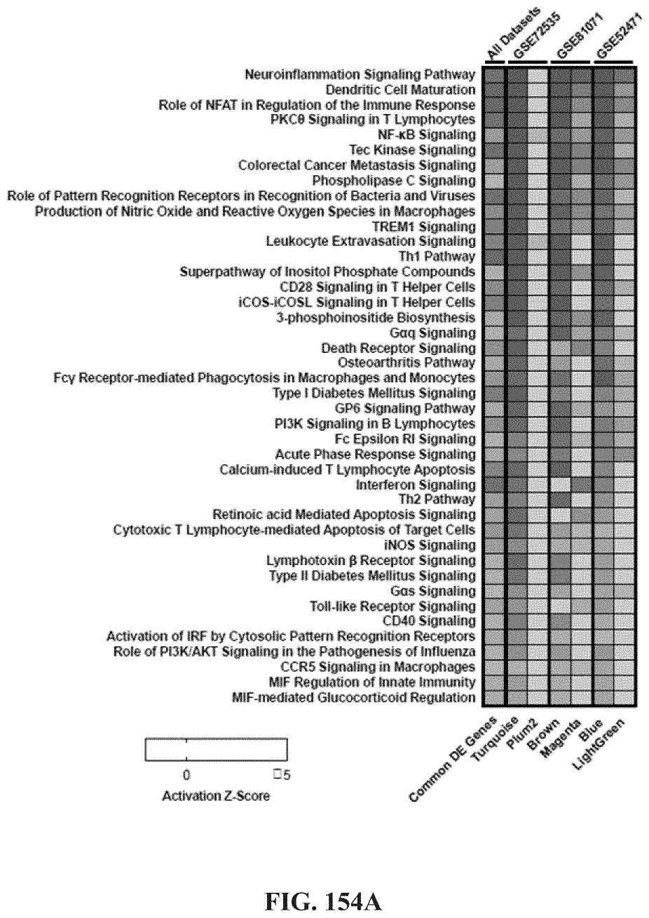

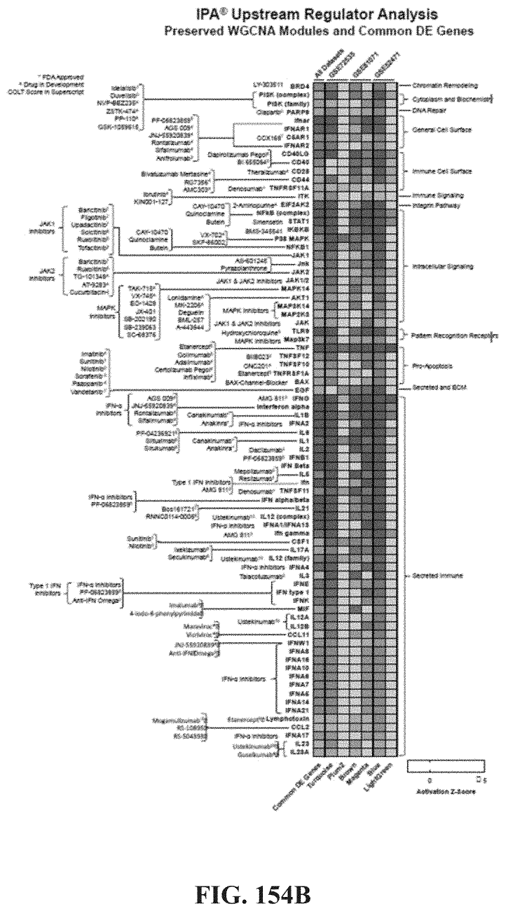

FIGS. 154A-154B show results of IPA® canonical pathway and upstream regulator (UR) analysis. IPA® canonical pathway and upstream regulator analysis was performed. The analysis compared DE genes common to all three datasets and the 6 preserved DLE-associated WGCNA modules. FIG. 154A shows canonical pathways predicted to be significantly activated or inhibited in both DE transcripts and at least one module from each dataset. FIG. 154B shows that a total of 224 URs were significantly activated or inhibited in both the DE transcripts and at least one module from each dataset. The 84 URs targeted by existing drugs are shown and organized by BIG-C™ category. Canonical pathways and upstream regulators were considered significant if |Activation Z-Score|≥2.

In conclusion, WGCNA identified several modules in each dataset that significantly correlated to disease. Notably, two positively correlated modules in each dataset were significantly preserved across all three analyses. Chemokines and pathways that mediate lymphocyte proliferation, organization and/or recruitment into DLE cutaneous tissue were detected as enriched via IPA® analysis, highlighting critical angles of therapeutic attack. Specifically, several IPA® URs were also high priority drug targets such as IFNγ, CD40, IL12, TNFRSF1A, IFNα, and JAK/STAT pathways that may prove to be good options for therapeutic intervention.

While preferred embodiments have been shown and described herein, it will be obvious to those skilled in the art that such embodiments are provided by way of example only. Numerous variations, changes, and substitutions will now occur to those skilled in the art without departing from the scope of the disclosure. It should be understood that various alternatives to the embodiments described herein may be employed in practice. Numerous different combinations of embodiments described herein are possible, and such combinations are considered part of the present disclosure. In addition, all features discussed in connection with any one embodiment herein can be readily adapted for use in other embodiments herein. It is intended that the following claims define the scope of the disclosure and that methods and structures within the scope of these claims and their equivalents be covered thereby.

What is claimed is:

1. A method of identifying one or more records having a specific phenotype, the method comprising:
(a) receiving a plurality of first records, wherein each first record is associated with one or more of a plurality of phenotypes;
(b) receiving a plurality of second records, wherein each second record is associated with one or more of the plurality of phenotypes, and wherein the plurality of second records and the plurality of first records are non-overlapping;
(c) applying a machine learning algorithm to at least one first record and at least one second record to determine a classifier;
(d) receiving a plurality of third records, wherein the third records are distinct from the plurality of first records and the plurality of second records; and
(e) applying the classifier to the plurality of third records to identify one or more third records associated with the specific phenotype.

2. The method of claim 1, wherein the first records and the second records comprise nucleic acid sequencing data, transcriptome data, genome data, epigenome data, proteome data, metabolome data, virome data, metabolome data, methylome data, lipidomic data, lineage-ome data, nucleosomal occupancy data, a genetic variant, a gene fusion, an indel, or any combination thereof.

3. The method of claim 1 or 2, wherein the first records and the second records are in different formats.

4. The method of any one of claims 1-3, wherein the first records and the second records are from different sources, different studies, or both.

5. The method of any one of claims 1-4, wherein the phenotype comprises a disease state, an organ involvement, a medication response, or any combination thereof.

6. The method of any one of claims 1-5, wherein the classifier comprises an elastic generalized linear model classifier, a k-nearest neighbors classifier, a random forest classifier, or any combination thereof.

7. The method of any one of claims 1-6, wherein applying a machine learning algorithm to the third data set comprises applying a machine learning algorithm to a plurality of unique third data sets.

8. The method of any one of claims 1-7, further comprising filtering the first records, the second records, or both.

9. The method of claim 8, wherein the filtering comprises removing outliers, removing background noise, removing data without annotation data, normalizing, scaling, variance correcting, Weighted Gene Co-expression Network Analysis, enrichment analysis, dimensionality reduction, or any combination thereof.

10. The method of claim 9, wherein the normalizing is performed by Robust Multi-Array Analysis (RMA), Guanine Cytosine Robust Multi-Array Analysis (GCRMA), Linear Models for Microarray Data, variance stabilizing transformation (VST), normal-exponential quantile correction (NEQC), or any combination thereof.

11. The method of claim 10, wherein the Weighted Gene Co-expression Network Analysis comprises calculating a topology matrix, clustering the data based on the topology matrix, and correlating module eigenvalues for traits on a linear scale by Pearson correlation, for nonparametric traits by Spearman correlation, and for dichotomous traits by point-biserial correlation or t-test.

12. The method of any one of claims 1-11, wherein the one or more records having a specific phenotype correspond to one or more subjects, and wherein the method further comprises identifying the one or more subjects as (i) having a diagnosis of a lupus condition, (ii) having a prognosis of a lupus condition, (iii) being suitable or not suitable for enrollment in a clinical trial for a lupus condition, (iv) being suitable or not suitable for being administered a therapeutic regimen configured to treat a lupus condition, (v) having an efficacy or not having an efficacy of a therapeutic regimen configured to treat a lupus condition, based at least in part on the specific phenotype corresponding to the one or more subjects.

13. A method for identifying a disease state or a susceptibility thereof of a subject, comprising:
(a) using an assay to process a biological sample derived from the subject to generate a quantitative measure of each of a plurality of disease-associated genomic loci, wherein the plurality of disease-associated genomic loci comprises one or more genes associated with a gene cluster of Table 1 to Table 72C;
(b) processing the dataset to identify the disease state or the susceptibility thereof of the subject at an accuracy of at least about 70%; and
(c) electronically outputting a report indicative of the disease state or the susceptibility thereof of the subject.

14. The method of claim 13, wherein the plurality of quantitative measures comprises gene expression measurements.

15. The method of claim 13, wherein the disease state comprises an active lupus condition or an inactive lupus condition.

16. The method of claim 15, wherein the lupus condition is systemic lupus erythematosus (SLE), discoid lupus erythematosus (DLE), or lupus nephritis (LN).

17. The method of claim 13, wherein the plurality of disease-associated genomic loci comprises 5, 10, 15, 20, 25, 30, 35, 40, 45, 50, or more than 50 genes associated with the gene cluster.

18. A method for identifying an immunological state of a subject, comprising:
(a) using an assay to process a biological sample derived from the subject to generate a quantitative measure of each of a plurality of genomic loci, wherein the plurality of genomic loci comprises one or more genes associated with a gene cluster of Table 1 to Table 72C;
(b) processing the dataset to identify the immunological state of the subject at an accuracy of at least about 70%; and
(c) electronically outputting a report indicative of the immunological state of the subject.

19. The method of claim 18, wherein the plurality of quantitative measures comprises gene expression measurements.

20. The method of claim 18, wherein the immunological state comprises an active lupus condition or an inactive lupus condition.

21. The method of claim 20, wherein the lupus condition is systemic lupus erythematosus (SLE), discoid lupus erythematosus (DLE), or lupus nephritis (LN).

22. The method of claim 18, wherein the plurality of disease-associated genomic loci comprises 5, 10, 15, 20, 25, 30, 35, 40, 45, 50, or more than 50 genes associated with the gene cluster.

23. A method for identifying an immunological state of a subject, comprising:
(d) using an assay to process a biological sample derived from the subject to generate a quantitative measure of each of a plurality of genomic loci, wherein the plurality of genomic loci comprises one or more genes associated with a pathway of Table 1 to Table 72C;
(e) processing the dataset to identify the immunological state of the subject at an accuracy of at least about 70%; and
(f) electronically outputting a report indicative of the immunological state of the subject.

24. The method of claim 23, wherein the plurality of quantitative measures comprises gene expression measurements.

25. The method of claim 23, wherein the disease state comprises an active lupus condition or an inactive lupus condition.

26. The method of claim 25, wherein the lupus condition is systemic lupus erythematosus (SLE), discoid lupus erythematosus (DLE), or lupus nephritis (LN).

27. The method of claim 23, wherein the plurality of disease-associated genomic loci comprises 5, 10, 15, 20, 25, 30, 35, 40, 45, 50, or more than 50 genes associated with the pathway.
